(19) United States
(12) Patent Application Publication
(10) Pub. No.: US 2026/0028365 A1
(43) Pub. Date: Jan. 29, 2026

(54) TYROSINE KINASE 2 INHIBITORS AND USES THEREOF

(71) Applicant: BIOGEN MA INC., Cambridge, MA (US)

(72) Inventors: Jeffrey Vessels, Marlborough, MA (US); Tamara Halkina Levin, Belmont, MA (US); Harold George Vandeveer, Stoneham, MA (US); Felix Gonzalez Lopez de Turiso, San Diego, CA (US); Zhili Xin, Cambridge, MA (US); Edward Yin Shiang Lin, Ashland, MA (US); Soma Maitra, Cambridge, MA (US); Vatee Pattaropong, Bedford, MA (US); Simone Sciabola, Wellesley, MA (US); Christopher Helal, Cambridge, MA (US); Kevin M. Guckian, Northborough, MA (US)

(21) Appl. No.: 18/994,337

(22) PCT Filed: Jul. 13, 2023
(86) PCT No.: PCT/US2023/027603

§ 371 Date: Jan. 14, 2025

Related U.S. Application Data
(60) Provisional application No. 63/389,038, filed on Jul. 14, 2022.

(51) Int. Cl. C07F 9/6558; A61K 31/444; A61K 31/497; A61K 31/501; A61K 31/506; A61K 31/513; A61K 31/519; A61K 31/53; A61K 31/5365; A61K 31/5377; A61K 31/5383; A61K 31/5386; A61K 31/675; C07B 59/00; C07D 213/75; C07D 401/12; C07D 401/14; C07D 405/14; C07D 413/14; C07D 417/14; C07D 471/04; C07D 487/04; C07D 491/048; C07D 491/052; C07D 491/056; C07D 491/08; C07D 491/107; C07D 498/04; C07D 498/08; C07D 513/04

(52) U.S. Cl. CPC C07F 9/65583; A61K 31/444; A61K 31/497; A61K 31/501; A61K 31/506; A61K 31/513; A61K 31/519; A61K 31/53; A61K 31/5365; A61K 31/5377; A61K 31/5383; A61K 31/5386; A61K 31/675; C07B 59/002; C07D 213/75; C07D 401/12; C07D 401/14; C07D 405/14; C07D 413/14; C07D 417/14; C07D 471/04; C07D 487/04; C07D 491/048; C07D 491/052; C07D 491/056; C07D 491/08; C07D 491/107; C07D 498/04; C07D 498/08; C07D 513/04; C07B 2200/05

(57) ABSTRACT

This disclosure relates to compounds of Formula (I) or pharmaceutically acceptable salts thereof, in which all of the variables in Formula (I) are as defined in the application. The compounds of this disclosure are capable of inhibiting the activity of tyrosine kinase 2 (TYK2). The disclosure further provides methods of preparing the compounds of the disclosure, and methods for their therapeutic use.

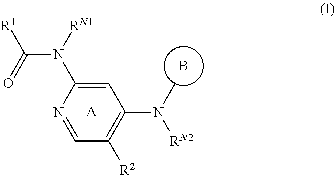

### RELATED APPLICATIONS

This application claims priority to U.S. Provisional Application No. 63/389,038, filed on Jul. 14, 2022, The entire contents of the foregoing application are expressly incorporated herein by reference.

### FIELD OF THE INVENTION

The present disclosure relates to inhibitors of Tyrosine kinase 2 (TYK2), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the TYK2 inhibitor to a warm-blooded animal, especially a human.

### BACKGROUND OF THE INVENTION

Cytokines are small secreted proteins released by cells and have a specific effect on the interactions and communications between cells. Cytokine pathways mediate a broad range of biological functions including many aspects of inflammation and immunity through mostly extracellular signaling.

Tyrosine kinase 2 (TYK2) is a member of Janus kinases (JAK) that are cytoplasmic protein kinases associated with cytokine receptors and play a central role in mediating cytokine signaling (Kisseleva et al., Gene, 2002, 285, 1; and Yamaoka et al. Genome Biology 2004, 5, 253). The JAK family also includes JAK1, JAK2 and JAK3. More specifically, cytokine's engagement with cognate receptors triggers activation of receptors associate with JAK, which leads to JAK mediated tyrosine phosphorylation of signal transducer and activator of transcription (STAT) proteins and ultimately transcriptional activation of specific gene sets (Schindler et al, 2007, J. Biol. Chem. 282: 20059-63). Numerous cytokines known to activate the JAK family include the interferon (IFN) family (IFN-alpha, IFN-beta, IFN-omega, Limitin, IFN-gamma, IL-10, IL-19, IL-20, IL-22), the glycoprotein (gp) 130 family (IL-6, IL-11, OSM, L1F, CNTF, NNT-1/BSF-3, G-CSF, CT-1, Leptin, IL-12, IL-23), the gamma C family (IL-2, IL-7, TSLP, IL-9, IL-15, IL-21, IL-4, IL-13), IL-3 family (IL-3, IL-5, GM-CSF), the single chain family (EPO, GH, PRL, TPO), receptor tyrosine kinases (EGF, PDGF, CSF-1, HGF), and G-protein coupled receptors (AT1).

TYK2 is important in the signaling of the type I interferons (e.g., IFN-alpha), IL-6, IL-10, IL-12 and IL-23 (Liang, Y. et al., Expert Opinion on Therapeutic Targets, 2014, 18,5, 571-580; Kisseleva et al., 2002, Gene 285:1-24; and Watford, W. T. & O'Shea, J. J., 2006, Immunity 25:695-697). Consistent with this, primary cells derived from a TYK2 deficient human are defective in type I interferon, IL-6, IL-10, IL-12 and IL-23 signaling. TYK2 signals with other members of the JAK family in the following combinations: TYK2/JAK1, TYK2/JAK2, TYK2/JAK1/JAK2.

Studies have shown that inappropriate JAK activities can arise from mutation, over-expression, or inappropriate regulation, dys-regulation or de-regulation, as well as over- or under-production of growth factors or cytokines, and therefore trigger a variety of biological cellular responses relating to cell growth, cell differentiation, cell function, survival, apoptosis, and cell mobility. The inappropriate JAK activities are implicated in many diseases that include but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease.

Small molecule JAK inhibitors have emerged as a major therapeutic advancement in treating autoimmune diseases. To date, all known small molecule JAK inhibitors that have progressed into development are active site-directed inhibitors that bind to the adenosine triphosphate (ATP) site of the catalytic domain (also referred to as the JH1 or “Janus Homology 1” domain) of the JAK protein, which prevents catalytic activity of the kinase by blocking ATP, downstream phosphorylation, and resulting pathway signal transduction (Bryan et al., J. Med. Chem. 2018, 61, 9030-9058).

Because of the high homology of the ATP active site across the kinome and especially within the JAK family, it is a significant challenge to achieve high selectivity for a specific JAK family member while also maintaining selectivity within the kinome. As a result, many JAK inhibitors that have been developed are pan-JAK inhibitors or are modestly selective for one or more JAK family members. While these inhibitors have shown encouraging results in treating autoimmune diseases, undesirable side effects leading to a narrow therapeutic index have been observed and suggest the need for improved treatments.

TYK2 has been shown to be important in the differentiation and function of multiple cell types important in inflammatory disease and autoimmune disease including natural killer cells, B cells, and T helper cell types. Aberrant TYK2 expression is associated with multiple autoimmune or inflammatory conditions.

There remains a need for potent compounds that demonstrate high selectivity for TYK2 over other members of the JAK family as potential therapeutic agents for treating diseases or disorders that are responsive to TYK2 inhibition.

### SUMMARY OF THE INVENTION

The present disclosure provides compounds that are TYK2 inhibitors. In a first aspect, the present disclosure relates to compounds having the Formula I:

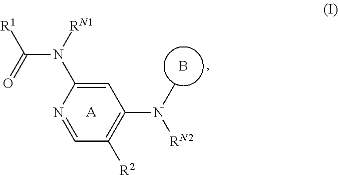

or a pharmaceutically acceptable salt thereof, wherein:
R¹ is H, C₁₋₆ alkyl, —OR^{1a}, —NR^{1b}R^{1c}, 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R^{1d};
   R^{1a}, R^{1b}, and R^{1c} are each independently H, C₁₋₄alkyl, or 3 to 4 membered monocyclic carbocyclyl;
   each R^{1d} is independently halo, oxo, —CN, —OR^{1a}, —NR^{1b}R^{1c} C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
R² is selected from H, halo, C₁₋₆alkyl, C₃₋₇cycloalkyl, —OR^{2a}, —N(R^{2b})₂, 4- to 11-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the C₁₋₆alkyl, C₃₋₇cycloalkyl, 5- to 11-membered monocyclic or bicyclic heterocyclyl, and 5- to 6-membered monocyclic heteroaryl represented by R² are each optionally substituted by 1 to 3 R²⁰;
   R^{2a} is selected from H, C₁₋₆alkyl, C₃₋₇cycloalkyl, 5- or 6-membered heteroaryl, and 4- to 7-membered monocyclic heterocyclyl having 1 to 4 heteroatoms independently selected from nitrogen, oxygen and sulfur, wherein the C₁₋₆alkyl, C₃₋₇cycloalkyl, 5- or 6-membered heteroaryl, and 4- to 7-membered monocyclic heterocyclyl represented by R^{2a} are optionally substituted with 1 to 3 R²⁰;
   each R^{2b} is independently H, C₁₋₄alkyl, C₁₋₃alkyl-C₁₋₃alkoxy, C₁₋₄alkoxy, or 4- to 6-membered monocyclic heterocyclyl;
   R²⁰, for each occurrence, is independently selected from halo, —CN, C₁₋₄alkyl, C₁₋₄ haloalkyl, —OR^{20c}—C(O)R^{20b}, —C(O)N(R^{20b})₂, —N(R^{20b})₂, —SO₂R^{20b}, —P(O)(C₁₋₃alkyl)₂, phenyl, C₃₋₇cycloalkyl, 5- to 10-membered bicyclic carbocycle, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the C₁₋₄alkyl, phenyl, C₃₋₇cycloalkyl, 5- to 10-membered bicyclic carbocycle, 4- to 10-membered monocyclic or bicyclic heterocyclyl and 5- to 6-membered monocyclic heteroaryl represented by R²⁰ are each optionally substituted with 1 to 3 R²⁰⁰;
   each R^{20b} is independently H, C₁₋₄alkyl or C₁₋₄alkoxy;
   R^{20c} is H, C₁₋₄alkyl, C₁₋₄haloalkyl, C₃₋₆cycloalkyl, or 4- to 6-membered monocyclic heterocyclyl, wherein the C₁₋₄alkyl is optionally substituted by C₁₋₃alkoxy;
   R²⁰⁰, for each occurrence, is independently selected from halo, —CN, C₁₋₄alkyl, C₁₋₃alkyl-C₁₋₃alkoxy, C₁₋₄haloalkyl, —OH, —N(R^{20b})₂, C₁₋₃alkoxy, C₁₋₃haloalkoxy, C₃₋₇cycloalkyl, and 5- to 10-membered monocyclic or bicyclic heterocyclyl optionally substituted with 1 to 3 C₁₋₃alkyl or C₁₋₃alkoxy;
Ring B is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{B};
   each R^{B} is independently selected from halo, —CN, —OR^{Ba}, —N(R^{Bb})₂, —C(O)R^{Bc}, —C(O)OR^{Ba}, —SO₂R^{Bc}, C₁₋₆alkyl, C₂₋₆alkyenyl, phenyl, 3 to 7 membered monocyclic carbocyclyl, 4- to 7-membered monocyclic or bicyclic heterocyclyl having 1 to 4 heteroatoms independently selected from nitrogen, oxygen and sulfur, and 5 to 10 membered monocyclic or bicyclic heteroaryl having 1 to 3 heteroatoms independently selected from nitrogen, oxygen and sulfur, wherein the C₁₋₆alkyl, C₂₋₆alkyenyl, phenyl, 3 to 7 membered monocyclic carbocyclyl, 4- to 7-membered monocyclic heterocyclyl and 5 to 10 membered monocyclic or bicyclic heteroaryl represented by R^{B} are each optionally substituted by one or more R^{B1};
   each R^{B1} is independently selected from halo, oxo, —CN, —OR^{Ba}, —N(R^{Bb})₂, C₁₋₄alkyl, C₁₋₄alkyl-R^{Bd}, C₁₋₄haloalkyl, —C(O)OR^{Ba}, phenyl, 5 to 6 membered heteroaryl, 3 to 7 membered monocyclic carbocyclyl, and 4 to 8 membered monocyclic heterocyclyl;
   R^{Ba} is independently H, C₁₋₄alkyl, C₃₋₇cycloalkyl, or 4- to 8-membered monocyclic or bicycle heterocyclyl, wherein the C₁₋₄alkyl, C₃₋₇cycloalkyl and 4- to 8-membered monocyclic or bicycle heterocyclyl represented by R^{Ba} are each optionally substituted with 1 or 2 R^{B0};
   each R^{B0} is independently halo, —CN, —OH, C₁₋₄alkyl or C₁₋₄alkoxy;
   each R^{Bb} is independently H, C₁₋₄alkyl, C₁₋₄alkoxy or C₃₋₇cycloalkyl;
   R^{Bc} is C₁₋₆alkyl or C₃₋₇cycloalkyl;
   R^{Bd} is —C(O)OR^{Ba}, —N(R^{Bb})₂, —OR^{Ba}, 3 to 7 membered monocyclic carbocyclyl, or 4 to 8 membered monocyclic heterocyclyl; and
R^{N1} and R^{N2} are each independently H or C₁₋₄alkyl.

Another aspect of the disclosure relates to pharmaceutical compositions comprising compounds of Formula (I) or pharmaceutically acceptable salts thereof, and a pharmaceutically acceptable carrier.

In yet another aspect, the present disclosure provides a method of treating a disease or disorder that is responsive to inhibition of TYK2 in a subject comprising administering to said subject an effective amount of at least one compound described herein or a pharmaceutically acceptable salt thereof.

Another aspect of the present disclosure relates to the use of at least one compound described herein or a pharmaceutically acceptable salt thereof, for the manufacture of a medicament for the treatment of a disease or disorder responsive to inhibition of TYK2. Also provided is a compound described herein or a pharmaceutically acceptable salt thereof for use in treating a disease or disorder responsive to inhibition of TYK2. Use of a compound described herein or a pharmaceutically acceptable salt thereof for treating a disease or disorder responsive to inhibition of TYK2 is also included in the present disclosure.

### DETAILED DESCRIPTION OF THE INVENTION

The present disclosure provides compounds and pharmaceutical compositions thereof that may be useful in the treatment of diseases or disorders through mediation of TYK2. In some embodiments, the compounds of present disclosure are TYK2 inhibitors.

### Compounds and Compositions

In a first embodiment, the present disclosure relates to compounds having the Formula I:

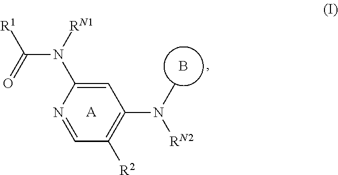

or a pharmaceutically acceptable salt thereof, wherein:
R¹ is H, C₁₋₆ alkyl, —OR^{1a}, —NR^{1b}R^{1c} 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R^{1d};
R^{1a}, R^{1b}, and R^{1c} are each independently H, C₁₋₄alkyl, or 3 to 4 membered monocyclic carbocyclyl;
each R^{1d} is independently halo, oxo, —CN, —OR^{1a}, —NR^{1b}R^{1c} C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
R² is selected from H, C₁₋₆alkyl, C₃₋₇cycloalkyl, —OR^{2a}, —N(R^{2b})₂, 4- to 11-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the C₁₋₆alkyl, C₃₋₇cycloalkyl, 5- to 11-membered monocyclic or bicyclic heterocyclyl, and 5- to 6-membered monocyclic heteroaryl represented by R² are each optionally substituted by 1 to 3 R²⁰;
R^{2a} is selected from H, C₁₋₆alkyl, C₃₋₇cycloalkyl and 4- to 7-membered monocyclic heterocyclyl having 1 to 4 heteroatoms independently selected from nitrogen, oxygen and sulfur, wherein the C₁₋₆alkyl, C₃₋₇cycloalkyl and 4- to 7-membered monocyclic heterocyclyl represented by R^{2a} are optionally substituted with 1 to 3 R²⁰;
each R^{2b} is independently H, C₁₋₄alkyl or C₁₋₄alkoxy;
R²⁰, for each occurrence, is independently selected from halo, —CN, C₁₋₄alkyl, C₁₋₄haloalkyl, C₁₋₄alkoxy, —N(R^{20b})₂, phenyl, C₃₋₇cycloalkyl, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the phenyl, C₃₋₇cycloalkyl, 4- to 10-membered monocyclic or bicyclic heterocyclyl and 5- to 6-membered monocyclic heteroaryl represented by R²⁰ are each optionally substituted with 1 to 3 R²⁰⁰;
each R^{20b} is independently H, C₁₋₄alkyl or C₁₋₄alkoxy;
R²⁰⁰, for each occurrence, is independently selected from halo, —CN, C₁₋₄alkyl, C₁₋₄haloalkyl, C₁₋₃alkoxy and C₃₋₇cycloalkyl; Ring B is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{B};
each R^{B} is independently selected from halo, —CN, —OR^{Ba}, —N(R^{Bb})₂, —C(O)R^{Bc}, —C(O)OR^{Ba}, —SO₂R^{Bc}, C₁₋₆alkyl, phenyl, 3 to 7 membered monocyclic carbocyclyl 4- to 7-membered monocyclic heterocyclyl having 1 to 4 heteroatoms independently selected from nitrogen, oxygen and sulfur, and 5 to 10 membered monocyclic or bicyclic heteroaryl having 1 to 3 heteroatoms independently selected from nitrogen, oxygen and sulfur, wherein the C₁₋₆ alkyl, phenyl, 3 to 7 membered monocyclic carbocyclyl, 4- to 7-membered monocyclic heterocyclyl and 5 to 10 membered monocyclic or bicyclic heteroaryl represented by R^{B} are each optionally substituted by one or more R^{B1};
each R^{B1} is independently selected from halo, oxo, —CN, —OR^{Ba}, —N(R^{Bb})₂, C₁₋₄alkyl, C₁₋₄alkyl-R^{Bd}, C₁₋₄haloalkyl, —C(O)OR^{Ba}, phenyl, 5 to 6 membered heteroaryl, 3 to 7 membered monocyclic carbocyclyl, and 4 to 8 membered monocyclic heterocyclyl;
R^{Ba} is independently H, C₁₋₄alkyl or C₃₋₇cycloalkyl, wherein the C₁₋₄alkyl and C₃₋₇cycloalkyl represented by R^{Ba} are each optionally substituted with 1 or 2 R^{B0};
each R^{B0} is independently halo, —CN, —OH, C₁₋₄alkyl or C₁₋₄alkoxy;
each R^{Bb} is independently H, C₁₋₄alkyl, C₁₋₄alkoxy or C₃₋₇cycloalkyl;
R^{Bc} is C₁₋₆alkyl or C₃₋₇cycloalkyl;
R^{Bd} is —C(O)OR^{Ba}, —N(R^{Bb})₂, —OR^{Ba}, 3 to 7 membered monocyclic carbocyclyl, or 4 to 8 membered monocyclic heterocyclyl; and
R^{N1} and R^{N2} are each independently H or C₁₋₄alkyl.

In a second embodiment, for the compounds of Formula (I), or a pharmaceutically acceptable salt thereof, Ring B is selected from phenyl, pyridinyl, pyrimidinyl and thiazolyl, each of which is substituted with one to three R^{B}; and the remaining variables are as described in the first aspect or the first embodiment.

In a third embodiment, for the compounds of Formula (I), or a pharmaceutically acceptable salt thereof, R^{N1} and R^{N2} are each independently H or —CH₃; and the remaining variables are as described in the first aspect or the first aspect or second embodiment.

In a fourth embodiment, the compound of the present disclosure is represented by Formula (II) or (III):

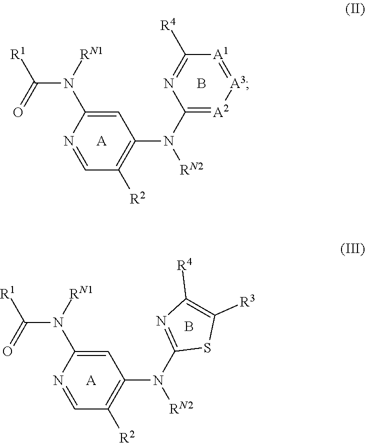

or a pharmaceutically acceptable salt thereof, wherein:
A¹ is N or CRS, A² is N or CR⁶, and A³ is N or CR³, provided no more than one of A¹, A², and A³ is N;
R³ is selected from H, —OR^{3a}, —N(R^{3b})₂, C₁₋₆alkyl, C₁₋₄haloalkyl, C₃₋₇cycloalkyl, phenyl, and 5 to 10 membered monocyclic or bicyclic heteroaryl having 1 to 3 heteroatoms independently selected from nitrogen, oxygen and sulfur, wherein the phenyl and 5 to 10 membered monocyclic or bicyclic heteroaryl are each optionally substituted by 1 to 3 R^{3c}; R^{3a} is H, C₁₋₄alkyl or C₃₋₇cycloalkyl, each of which is optionally substituted with 1 or 2 R³⁰;
   each R³⁰ is independently halo, —CN, —OH, C₁₋₄alkyl or C₁₋₄alkoxy;
   each R^{3b} is independently H, C₁₋₄alkyl, C₁₋₄alkoxy or C₃₋₇cycloalkyl;
   each R^{3c} is independently selected from halo, oxo, —CN, —OR^{3a}, —N(R^{3b})₂, C₁₋₄ alkyl, C₁₋₄alkyl-R^{3d}, C₁₋₄haloalkyl, —C(O)OR^{3a}, phenyl, 5 to 6 membered heteroaryl, 3 to 7 membered monocyclic carbocyclyl, and 4 to 8 membered monocyclic heterocyclyl;
   R^{3d} is —C(O)OR^{3a}, —N(R^{3b})₂, —OR^{3a}, 3 to 7 membered monocyclic carbocyclyl, or 4 to 8 membered monocyclic heterocyclyl;
R⁴ is selected from H, C₁₋₆alkyl, C₁₋₆haloalkyl, C₁₋₄alkoxy, —SO₂R^{4a}, 4- to 6-membered monocyclic heterocyclyl having 1 to 2 heteroatoms selected from nitrogen and oxygen and C₃₋₇cycloalkyl optionally substituted with 1 to 3 substituents independently halo or C₁₋₄alkyl;
R^{4a} is C₁₋₆alkyl;
R⁵ is H, halo, C₁₋₃alkyl, C₁₋₃haloalkyl or 5- to 6-membered heteroaryl having 1 to 3 heteroatoms independently selected from nitrogen, oxygen and sulfur, wherein the 5- to 6-membered heteroaryl represented by R⁵ is optionally substituted by 1 to 3 R⁵⁰; and
R⁵⁰, for each occurrence, is independently halo, C₁₋₄alkyl or C₁₋₄haloalkyl; and
R⁶ is H, halo, C₁₋₃alkyl, C₁₋₃haloalkyl or C₁₋₄alkoxy; and the remaining variables are as described in the first aspect or the first, second, or third embodiment. In an alternative fourth embodiment, the compound of the present disclosure is represented by Formula (II), (II′), or (III):

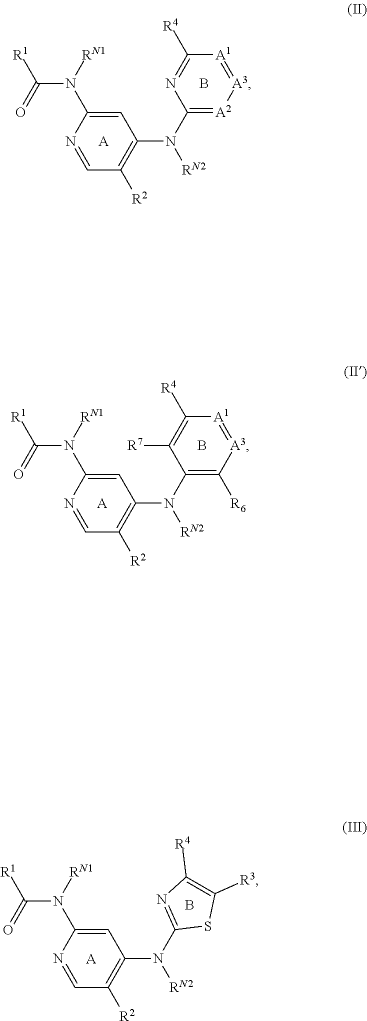

or a pharmaceutically acceptable salt thereof, wherein:
A¹ is N or CRS, A² is N or CR⁶, and A³ is N or CR³, provided no more than one of A¹, A², and A³ is N;
R³ is selected from H, halo, —OR^{3a}, —N(R^{3b})₂, C₁₋₆alkyl, C₁₋₄haloalkyl, C₁₋₃alkyl-C₁₋₃alkoxy, C₂alkeneyl, C₃₋₇cycloalkyl, phenyl, and 5 to 10 membered monocyclic or bicyclic heteroaryl having 1 to 3 heteroatoms independently selected from nitrogen, oxygen and sulfur, wherein the phenyl and 5 to 10 membered monocyclic or bicyclic heteroaryl are each optionally substituted by 1 to 3 R^{3c};
   R^{3a} is H, C₁₋₄alkyl, 4- to 8-membered monocyclic or bicycle heterocyclyl, or C₃₋₇cycloalkyl, each of which is optionally substituted with 1 or 2 R³⁰;
   each R³⁰ is independently halo, —CN, —OH, C₁₋₄alkyl or C₁₋₄alkoxy;
   each R^{3b} is independently H, C₁₋₄alkyl, C₁₋₄alkoxy or C₃₋₇cycloalkyl;
   each R^{3c} is independently selected from halo, oxo, —CN, —OR^{3a}, —N(R^{3b})₂, C₁₋₄ alkyl, C₁₋₄alkyl-R^{3d}, C₁₋₄haloalkyl, —C(O)OR^{3a}, phenyl, 5 to 6 membered heteroaryl, 3 to 7 membered monocyclic carbocyclyl, and 4 to 8 membered monocyclic heterocyclyl;
   R^{3d} is —C(O)OR^{3a}, —N(R^{3b})₂, —OR^{3a}, 3 to 7 membered monocyclic carbocyclyl, or 4 to 8 membered monocyclic heterocyclyl;
R⁴ is selected from H, C₁₋₆alkyl, C₁₋₆haloalkyl, C₁₋₄alkoxy, C₁₋₃alkoxy-C₁₋₃alkoxy, C₁₋₃ haloalkoxy, —C₂haloalkenyl, —SO₂R^{4a}, 4- to 8-membered monocyclic or bicyclic heterocyclyl having 1 to 2 heteroatoms selected from nitrogen and oxygen, and C₃₋₇cycloalkyl, wherein the 4- to 8-membered monocyclic or bicyclic heterocyclyl and C₃₋₆cycloalkyl are each optionally substituted with 1 to 3 substituents independently selected from halo, C₁₋₄alkyl, C₁₋₃ haloalkyl, and C₁₋₃alkyl-C₁₋₃alkoxy;
   R^{4a} is C₁₋₆alkyl;
R⁵ is H, halo, C₁₋₃alkyl, C₁₋₃haloalkyl or 5- to 6-membered heteroaryl having 1 to 3 heteroatoms independently selected from nitrogen, oxygen and sulfur, wherein the 5- to 6-membered heteroaryl represented by R⁵ is optionally substituted by 1 to 3 R⁵⁰; and
   R⁵⁰, for each occurrence, is independently halo, C₁₋₄alkyl or C₁₋₄haloalkyl; and
R⁶ and R⁷ are each, independently, H, halo, C₁₋₃alkyl, C₁₋₃haloalkyl or C₁₋₄alkoxy; and the remaining variables are as described in the first aspect or the first, second, or third embodiment.

In a fifth embodiment, the compound of the present disclosure is represented by Formula (IV), (V), (VI), or (VII):

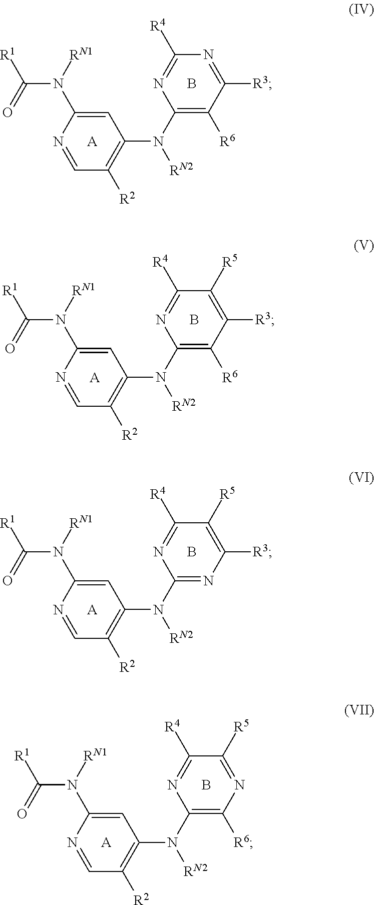

or a pharmaceutically acceptable salt thereof, wherein the variables in Formula (IV), (V), (VI), or (VII) are as defined in the first, second, or third embodiment. In an alternative fifth embodiment, the compound of the present disclosure is represented by Formula (IV), (V), (VI), (VII), (VIII), or (IX):

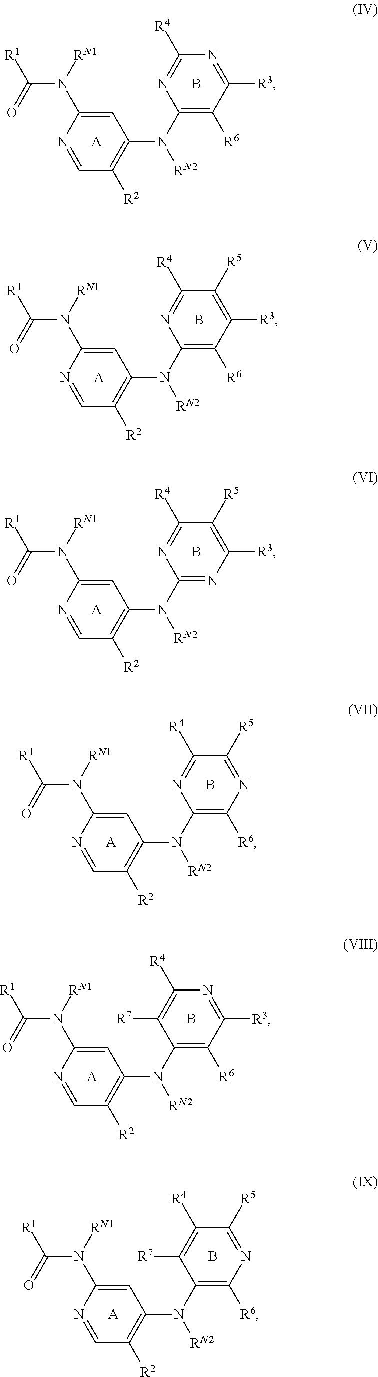

or a pharmaceutically acceptable salt thereof, wherein the variables in Formula (IV), (V), (VI), (VII), (VIII), or (IX) are as defined in the first, second, or third embodiment.

In a sixth embodiment, for the compounds of Formula (I), (II), (II′), (III), (IV), (V), (VI), (VII), (VIII) or (IX) or a pharmaceutically acceptable salt thereof, R¹ is C₁₋₄alkyl or C₃₋₆cycloalkyl, wherein the C₁₋₄alkyl is optionally substituted with C₁₋₃alkoxy; and the remaining variables are as described in the first aspect or the first, second, third, fourth, or fifth embodiment. In an alternative sixth embodiment, for the compounds of Formula (I), (II), (II′), (III), (IV), (V), (VI), (VII), (VIII), or (IX), or a pharmaceutically acceptable salt thereof, R¹ is H, C₁₋₄alkyl, —OR^{1a}, —NR^{1b}R^{1c} or C₃₋₆cycloalkyl, wherein the C₁₋₄alkyl is optionally substituted with C₁₋₃alkoxy; R^{1a} is C₁₋₃alkyl; R^{1b} and R^{1c} are each, independently, H or C₁₋₃alkyl; and the remaining variables are as described in the first aspect or the first, second, third, fourth, or fifth embodiment.

In a seventh embodiment, for the compounds of Formula (I), (II), (II′), (III), (IV), (V), (VI), (VII), (VIII) or (IX), or a pharmaceutically acceptable salt thereof, R¹ is selected from —CH₃, —CH₂CH₃, —CH₂OCH₃, —CH₂CH₂OCH₃ and cyclopropyl; and the remaining variables are as described in the first aspect or the first, second, third, fourth, or fifth embodiment. In an alternative seventh embodiment, for the compounds of Formula (I), (II), (II′), (III), (IV), (V), (VI), (VII), (VIII), or (IX), or a pharmaceutically acceptable salt thereof, R¹ is selected from H, —CH₃, —CH₂CH₃, —CH₂OCH₃, —CH₂CH₂OCH₃, —OCH₃, —NH₂, —NHCH₃ and cyclopropyl; and the remaining variables are as described in the first aspect or the first, second, third, fourth, or fifth embodiment.

In an eighth embodiment, for the compounds of Formula (I), (II), (II′), (III), (IV), (V), (VI), (VII), (VIII) or (IX), or a pharmaceutically acceptable salt thereof,
R² is selected from H, C₁₋₄alkyl, —OR^{2a}, and —N(R^{2b})₂, wherein the C₁₋₄alkyl represented by R² is optionally substituted with 1 to 3 R²⁰;
R^{2a} is H, C₁₋₄alkyl, C₃₋₆cycloalkyl or 4- to 6-membered monocyclic heterocyclyl having 1 to 4 heteroatoms independently selected from nitrogen, oxygen and sulfur, wherein the C₁. 4alkyl, C₃₋₆cycloalkyl and 4- to 6-membered monocyclic heterocyclyl represented by R^{2a} are each optionally substituted with 1 to 3 R²⁰;
R²⁰ is independently selected from halo, C₁₋₃alkyl, C₁₋₃alkoxy, —N(R^{20b})₂, phenyl, C₃₋₆ cycloalkyl, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 4 heteroatoms independently selected from nitrogen, oxygen and sulfur, wherein the phenyl, C₃₋₆cycloalkyl, 4- to 10-membered monocyclic or bicyclic heterocyclyl and 5- to 6-membered heteroaryl are each optionally substituted with 1 to 3 R²⁰⁰;
R^{2b}, for each occurrence, is independently H or C₁₋₃alkyl;
R^{20b}, for each occurrence, is independently H or C₁₋₃alkyl; and
R²⁰⁰, for each occurrence, is independently selected from halo, C₁₋₄alkyl, C₁₋₄haloalkyl, C₁₋₂alkoxy and C₃₋₅cycloalkyl; and the remaining variables are as described in the first aspect or the first, second, third, fourth, fifth, sixth, or seventh embodiment.

In an alternative eighth embodiment, for the compounds of Formula (I), (II), (II′), (III), (IV), (V), (VI), (VII), (VIII), or (IX), or a pharmaceutically acceptable salt thereof;
R² is selected from H, halo, C₁₋₄alkyl, —OR^{2a}, and —N(R^{2b})₂, wherein the C₁₋₄alkyl represented by R² is optionally substituted with 1 to 3 R²⁰;
R^{2a} is H, C₁₋₄alkyl, C₃₋₆cycloalkyl, 5- or 6-membered heteroaryl, or 4- to 6-membered monocyclic heterocyclyl having 1 to 4 heteroatoms independently selected from nitrogen, oxygen and sulfur, wherein the C₁₋₄alkyl, C₃₋₆cycloalkyl, 5- or 6-membered heteroaryl, and 4- to 6-membered monocyclic heterocyclyl represented by R^{2a} are each optionally substituted with 1 to 3 R²⁰;
R²⁰ is independently selected from halo, C₁₋₄alkyl, C₁₋₄alkoxy, —C(O)R^{20b}, —C(O)N(R^{20b})₂, —N(R^{20b})₂, phenyl, C₃₋₆cycloalkyl, 5- to 10-membered bicyclic carbocycle, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 4 heteroatoms independently selected from nitrogen, oxygen and sulfur, wherein the C₁₋₄ alkyl, phenyl, C₃₋₆cycloalkyl, 4- to 10-membered monocyclic or bicyclic heterocyclyl and 5- to 6-membered heteroaryl are each optionally substituted with 1 to 3 R²⁰⁰;
R^{2b}, for each occurrence, is independently H or C₁₋₃alkyl;
R^{20b}, for each occurrence, is independently H or C₁₋₃alkyl; and
R²⁰⁰, for each occurrence, is independently selected from halo, C₁₋₄alkyl, C₁₋₃alkyl-C₁₋₃alkoxy, C₁₋₄haloalkyl, C₁₋₂alkoxy, and C₃₋₅cycloalkyl.

In a ninth embodiment, for the compounds of Formula (I), (II), (II′), (III), (IV), (V), (VI), (VII), (VIII) or (IX), or a pharmaceutically acceptable salt thereof, R² is H, C₁₋₄alkyl, —OR^{2a} or —N(R^{2b})₂, wherein the C₁₋₄alkyl represented by R² is optionally substituted with 1 to 3 substituents independently selected from halo, C₁₋₃alkoxy and —N(R^{20b})₂; and R^{2a} is H or C₁₋₄ alkyl optionally substituted with 1 to 3 substituents independently selected from halo, C₁₋₃ alkoxy and —N(R^{20b})₂; and the remaining variables are as described in the eighth embodiment.

In a tenth embodiment, for the compounds of Formula (I), (II), (II′), (III), (IV), (V), (VI), (VII), (VIII) or (IX), or a pharmaceutically acceptable salt thereof, R² is —OR^{2a}; R^{2a} is C₃₋₆ cycloalkyl or 4- to 6-membered monocyclic heterocyclyl having 1 to 2 heteroatoms independently selected from nitrogen and oxygen, wherein the C₃₋₆cycloalkyl or 4- to 6-membered monocyclic heterocyclyl represented by R^{2a} are each optionally substituted with 1 to 3 substituents independently selected from halo, C₁₋₃alkyl and C₁₋₃alkoxy; and the remaining variables are as described in the eighth embodiment. In an alternative tenth embodiment, for the compounds of Formula (I), (II), (II′), (III), (IV), (V), (VI), (VII), (VIII), or (IX), R² is —OR^{2a}; R^{2a} is C₃₋₆cycloalkyl, 5- or 6-membered heteroaryl, or 4- to 6-membered monocyclic heterocyclyl having 1 to 2 heteroatoms independently selected from nitrogen and oxygen, wherein the C₃₋₆cycloalkyl, 5- or 6-membered heteroaryl, and 4- to 6-membered monocyclic heterocyclyl represented by R^{2a} are each optionally substituted with 1 to 3 substituents independently selected from halo, C₁₋₃alkyl and C₁₋₃alkoxy; and the remaining variables are as described in the eighth embodiment.

In an eleventh embodiment, for the compounds of Formula (I), (II), (II′), (III), (IV), (V), (VI), (VII), (VIII) or (IX), or a pharmaceutically acceptable salt thereof, R^{2a} is cyclopropyl, cyclobutyl, cyclopentyl, oxetanyl, azetidinyl, tetrahydrofuranyl or pyrrolidinyl, each of which is optionally substituted with 1 to 3 substituents independently selected from halo, C₁₋₃alkyl and C₁₋₃alkoxy; and the remaining variables are as described in the tenth embodiment. In an alternative eleventh embodiment, for the compounds of Formula (I), (II), (II′), (III), (IV), (V), (VI), (VII), (VIII), or (IX), R^{2a} is cyclopropyl, cyclobutyl, cyclopentyl, oxetanyl, azetidinyl, tetrahydrofuranyl pyrrolidinyl, pyrazinyl, pyridazinyl, or pyrazoyl, each of which is optionally substituted with 1 to 3 substituents independently selected from halo, C₁₋₃alkyl and C₁₋₃alkoxy; and the remaining variables are as described in the tenth embodiment.

In a twelfth embodiment, for the compounds of Formula (I), (II), (II′), (III), (IV), (V), (VI), (VII), (VIII) or (IX), or a pharmaceutically acceptable salt thereof, R^{2a} is represented by the following:

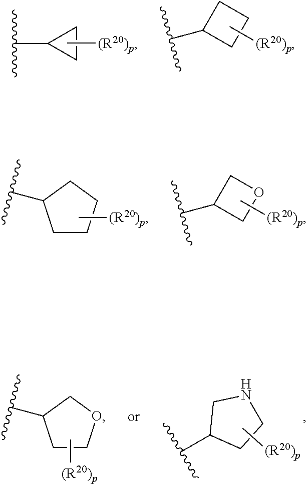

wherein p is 0, 1, 2 or 3; and each R²⁰ is independently halo, C₁₋₃alkyl and C₁₋₃alkoxy; and the remaining variables are as described in the tenth embodiment. In an alternative twelfth embodiment, for the compounds of Formula (I), (II), (II′), (III), (IV), (V), (VI), (VII), (VIII), or (IX), or a pharmaceutically acceptable salt thereof, R^{2a} is represented by the following:

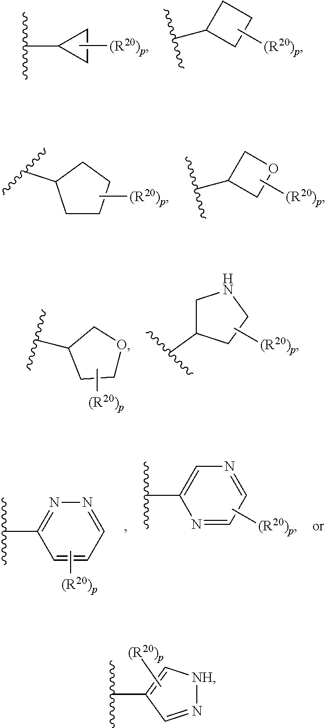

wherein p is 0, 1, 2 or 3; and each R²⁰ is independently halo, C₁₋₃alkyl and C₁₋₃alkoxy; and the remaining variables are as described in the tenth embodiment. In some embodiments, R^{2a} is represented by the following:

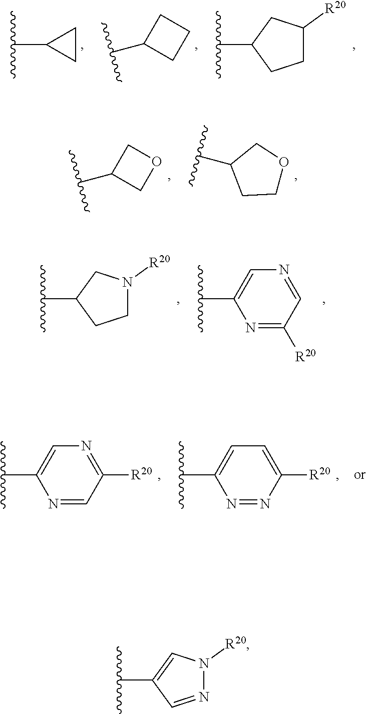

In a thirteenth embodiment, for the compounds of Formula (I), (II), (II′), (III), (IV), (V), (VI), (VII), (VIII) or (IX), or a pharmaceutically acceptable salt thereof, R^{2a} is represented by the following:

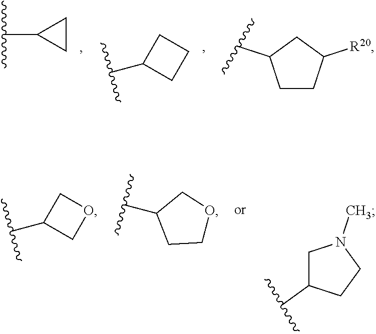

and the remaining variables are as described in the twelfth embodiment. In an alternative thirteenth embodiment, for the compounds of Formula (I), (II), (II′), (III), (IV), (V), (VI), (VII), (VIII), or (IX), or a pharmaceutically acceptable salt thereof, R^{2a} is represented by the following:

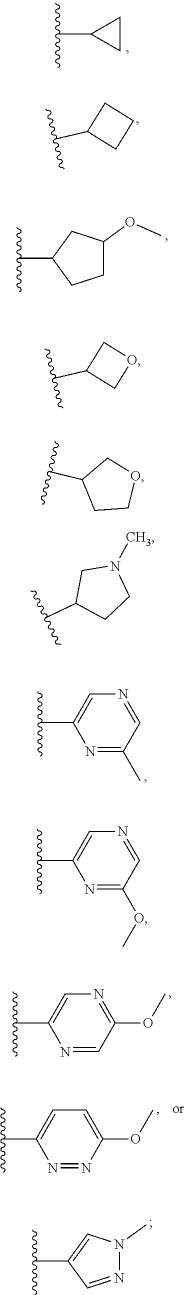

and the remaining variables are as described in the twelfth embodiment.

In a fourteenth embodiment, for the compounds of Formula (I), (II), (II′), (III), (IV), (V), (VI), (VII), (VIII) or (IX), or a pharmaceutically acceptable salt thereof, R² is —OR^{2a} and R^{2a} is C₁₋₄alkyl substituted with one R²⁰; and R²⁰ is phenyl, C₃₋₆cycloalkyl, 4- to 10-membered monocyclic or bicyclic heterocyclyl having 1 to 2 heteroatoms independently selected from nitrogen and oxygen, and 5- to 6-membered monocyclic heteroaryl having 1 to 3 heteroatoms independently selected from nitrogen, oxygen and sulfur, wherein the phenyl, C₃₋₆cycloalkyl, 4- to 10-membered monocyclic or bicyclic heterocyclyl and 5- to 6-membered monocyclic heteroaryl are each optionally substituted with 1 to 3 R²⁰⁰; and the remaining variables are as described in the eighth embodiment. In an alternative fourteenth embodiment, for the compounds of Formula (I), (II), (II′), (III), (IV), (V), (VI), (VII), (VIII), or (IX), or a pharmaceutically acceptable salt thereof, R² is —OR^{2a} and R^{2a} is C₁₋₄alkyl substituted with one R²⁰; and R²⁰ is phenyl, C₃₋₆cycloalkyl, 5- to 10-membered bicyclic carbocycle, 4- to 10-membered monocyclic or bicyclic heterocyclyl having 1 to 2 heteroatoms independently selected from nitrogen and oxygen, and 5- to 6-membered monocyclic heteroaryl having 1 to 3 heteroatoms independently selected from nitrogen, oxygen and sulfur, wherein the phenyl, C₃₋₆cycloalkyl, 5- to 10-membered bicyclic carbocycle, 4- to 10-membered monocyclic or bicyclic heterocyclyl and 5- to 6-membered monocyclic heteroaryl are each optionally substituted with 1 to 3 R²⁰⁰; and the remaining variables are as described in the eighth embodiment.

In a fifteenth embodiment, for the compounds of Formula (I), (II), (II′), (III), (IV), (V), (VI), (VII), (VIII) or (IX), or a pharmaceutically acceptable salt thereof, R²⁰ is independently selected from azetindinyl, benzo[d][1,3]dioxolyl, cyclobutyl, cyclopropyl, dihydrofuranonyl, imidazolyl, isoxazolyl, morpholinyl, oxabicyclo[2.2.1]hexanyl, oxadiazolyl, oxetanyl, oxazolyl, phenyl, pyrazolyl, pyridinyl, pyrrolidinyl and tetrahydrofuranyl, each of which is optionally substituted with 1 to 3 R²⁰⁰; and the remaining variables are as described in the fourteenth embodiment. In an alternative fifteenth embodiment, for the compounds of Formula (I), (II), (II′), (III), (IV), (V), (VI), (VII), (VIII), or (IX), or a pharmaceutically acceptable salt thereof, R²⁰ is independently selected from azetindinyl, benzo[d][1,3]dioxolyl, cyclobutyl, cyclopropyl, spiro[2.2]pentanyl, bicyclo[1.1.1]pentanyl, 2-oxabicyclo[2.1.1]hexanyl, 5-oxaspiro[2.4]heptanyl, 6-oxaspiro[3.4]octanyl, dihydrofuranonyl, 1,3-dioxolanyl, morpholinyl, piperazinyl, 1,4-dioxanyl, 5,8-dioxaspiro[3.5]nonanyl, tetrahydropyranyl, 3-oxabicyclo[3.1.1]heptanyl, 2-oxabicyclo[2.2.2]octanyl, 7-oxabicyclo[2.2.1]heptanyl, imidazolyl, isoxazolyl, morpholinyl, oxabicyclo[2.2.1]hexanyl, oxadiazolyl, oxetanyl, oxazolyl, phenyl, furanyl, thiazoyl, isothiazolyl, thiadiazolyl, triazoyl, pyrazolyl, pyridinyl, pyrimidinyl, pyrazinyl, pyrrolidinyl and tetrahydrofuranyl, each of which is optionally substituted with 1 to 3 R²⁰⁰; and the remaining variables are as described in the fourteenth embodiment.

In a sixteenth embodiment, for the compounds of Formula (I), (II), (II′), (III), (IV), (V), (VI), (VII), (VIII) or (IX), or a pharmaceutically acceptable salt thereof, R²⁰ is independently selected from:

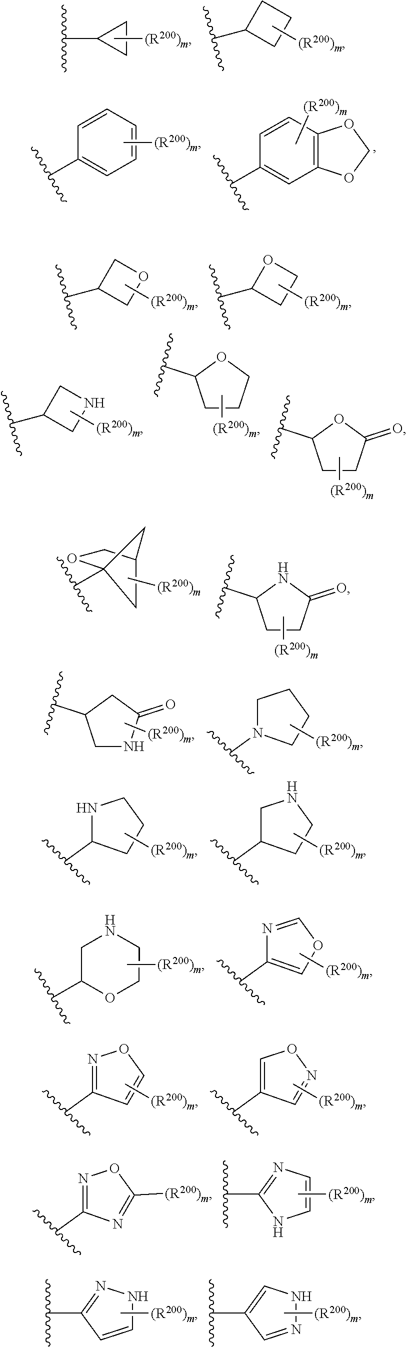

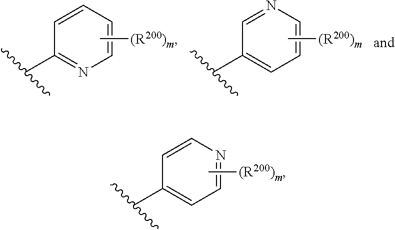

wherein m is 0, 1 or 2 as valence permits; and the remaining variables are as described in the fourteenth embodiment. In an alternative sixteenth embodiment, for the compounds of Formula (I), (II), (II′), (III), (IV), (V), (VI), (VII), (VIII), or (IX), or a pharmaceutically acceptable salt thereof, R²⁰ is independently selected from:

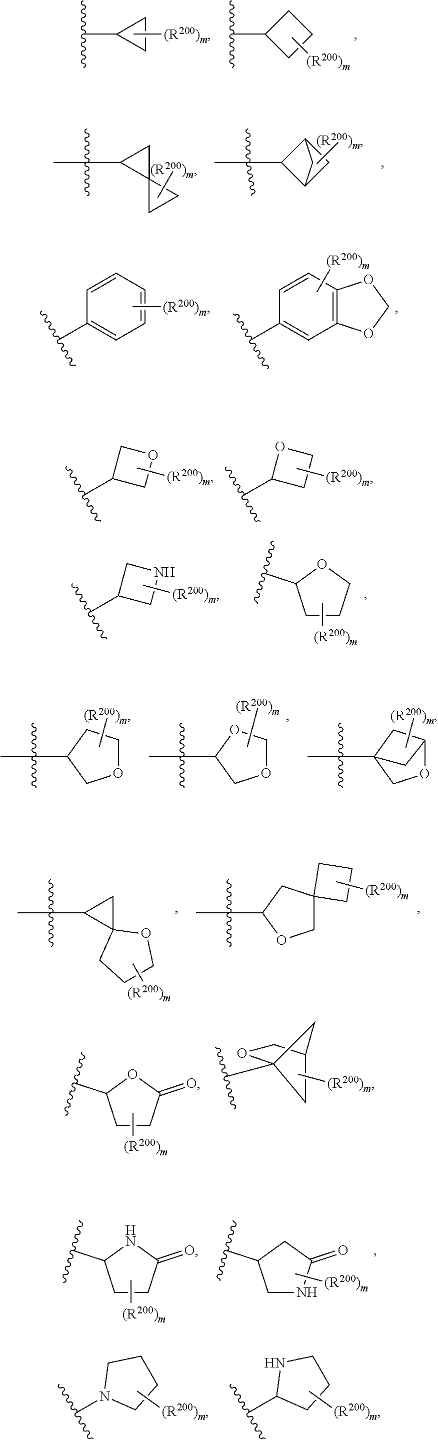

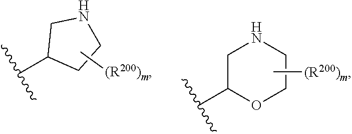

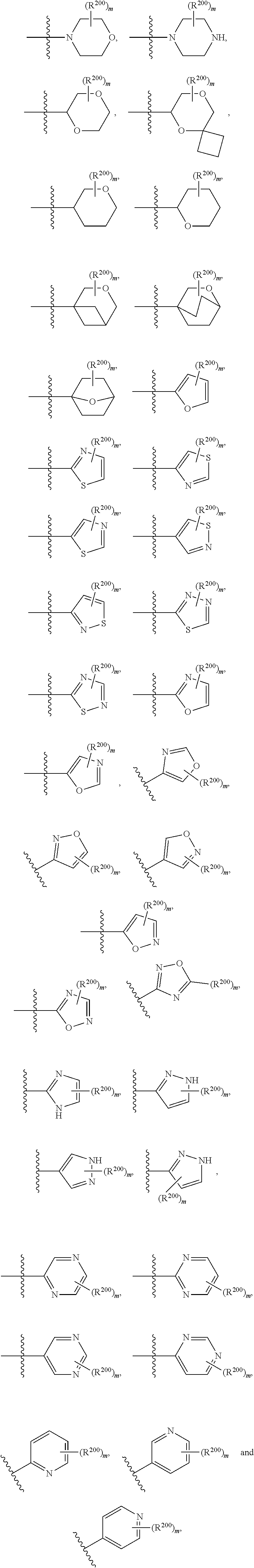

wherein m is 0, 1 or 2 as valence permits; and the remaining variables are as described in the fourteenth embodiment.

In a seventeenth embodiment, for the compounds of Formula (I), (II), (II′), (III), (IV), (V), (VI), (VII), (VIII) or (IX), or a pharmaceutically acceptable salt thereof, R² is selected from H, —CH₃, —CH₂CH₃, —CH₂CH₂CH₃, —CH₂CH₂CH₂OCH₃, —CH(CH₃)₂, —CH₂CH₂CH₂N(CH₃)₂, cyclopropyl, —CH₂OCH₃, OH, —OCH₃, —OCD₃, —OCHF₂, —OCH₂CH₃, —OCD₂CH₃, —OCD₂CD₃, —OCH₂CHF₂, —OCH₂CF₃, —OCH(CH₃)₂, —OCH₂CH₂OCH₃, —OCH₂CH₂OCF₂H, —OCH₂CH₂CH₂OCH₃, —OCH₂CH(CH₃)OCH₃, —OCH(CH₃)CH₂OCH₃, —OCH₂C(CH₃)₂OCH₃, —OCH₂CH₂OCH₂CH₃, —OCH₂CH₂OCH(CH₃)₂, —OCH₂CH₂N(CH₃)₂, —NH₂, —N(CH₃)₂,

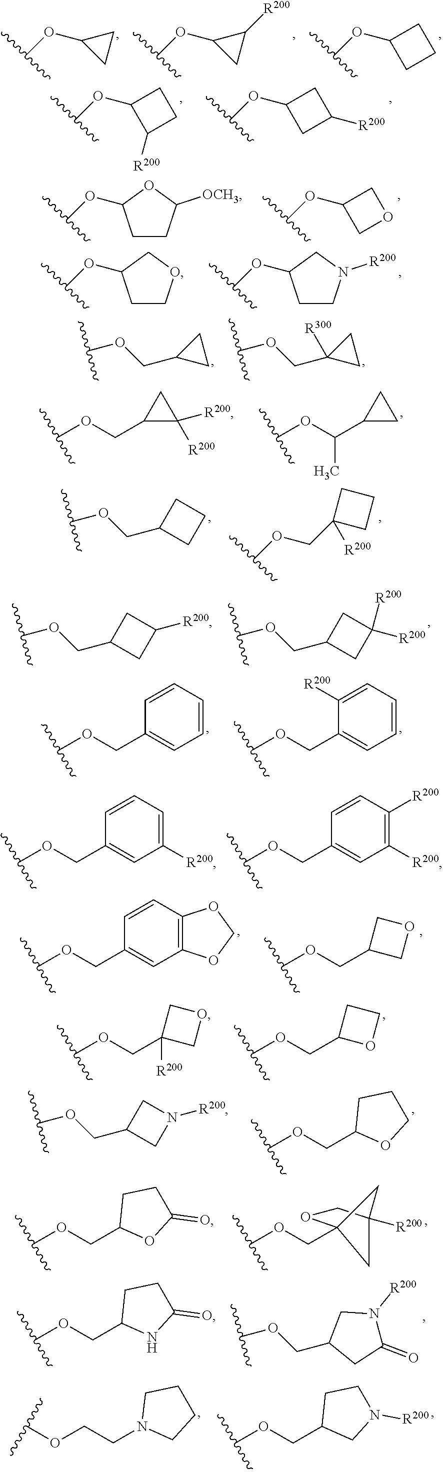

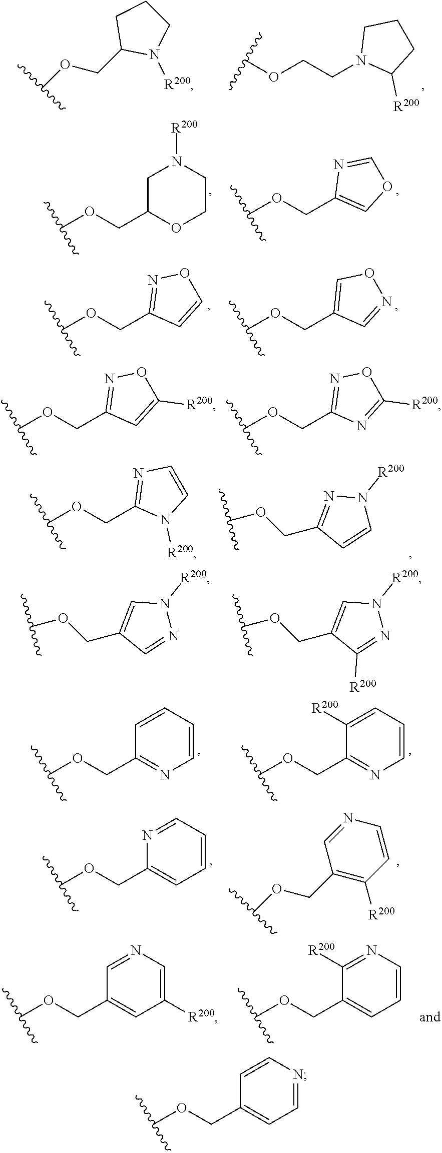

and the remaining variables are as described in the first aspect or the first, second, third, fourth, fifth, sixth, or seventh embodiment. In an alternative seventeenth embodiment, for the compounds of Formula (I), (II), (II′), (III), (IV), (V), (VI), (VII), (VIII), or (IX), or a pharmaceutically acceptable salt thereof, R² is selected from H, —F, —CH₃, —CH₂CH₃, —CH₂CH₂CH₃, —CF₂CH₂CH₃, —CH₂CH₂CH₂OCH₃, —CH(CH₃)₂, —CH₂CH₂CH₂N(CH₃)₂, —CF₂-cyclopropyl, cyclopropyl, —CH₂OCH₃, —OH, —OCH₃, —OCD₃, —OCHF₂, —OCH₂CH₃, —OCD₂CH₃, —OCD₂CD₃, —OCH₂CH₂F, —OCH₂CHF₂, —OCH₂CF₃, —OCH₂CH₂CH₂F, —OCH₂CH₂CH(CH₃)F, —OCH(CH₃)₂, —OCH₂CF(CH₃)₂, —OCH₂CH₂CH₂CH₃, —OCH₂CHFCH(CH₃)₂, —OCH₂CHF-cyclobutyl, —OCH₂CH₂OH, —OCH₂CH(OCH₃)CH₂CH₃, —OCH₂CH₂OCH₃, —OCH₂CH₂OCF₂H, —OCH₂CH₂OCH₂CH₃, —OCH₂CH₂CH₂OCH₃, —OCH₂CH(CH₃)OCH₃, —OCH₂CH(CH₃)CH₂OCH₃, —OCH(CH₃)CH₂OCH₃, —OCH₂CH(CH₃)OC(CH₃)₃, —OCH₂CH₂CH(CH₃)OCH₃, —OCH₂C(CH₃)₂OCH₃, —OCH₂CH₂OCH₂CH₃, —OCH₂CH₂OCH(CH₃)₂, —OCH₂CH₂OC(CH₃)₃, —OCH₂CH₂O-cyclopropyl, —OCH₂CH₂N(CH₃)₂, —OCH₂C(O)NHCH₃, —OCH₂C(O)N(CH₃)₂, —NH₂, —N(CH₃)₂,

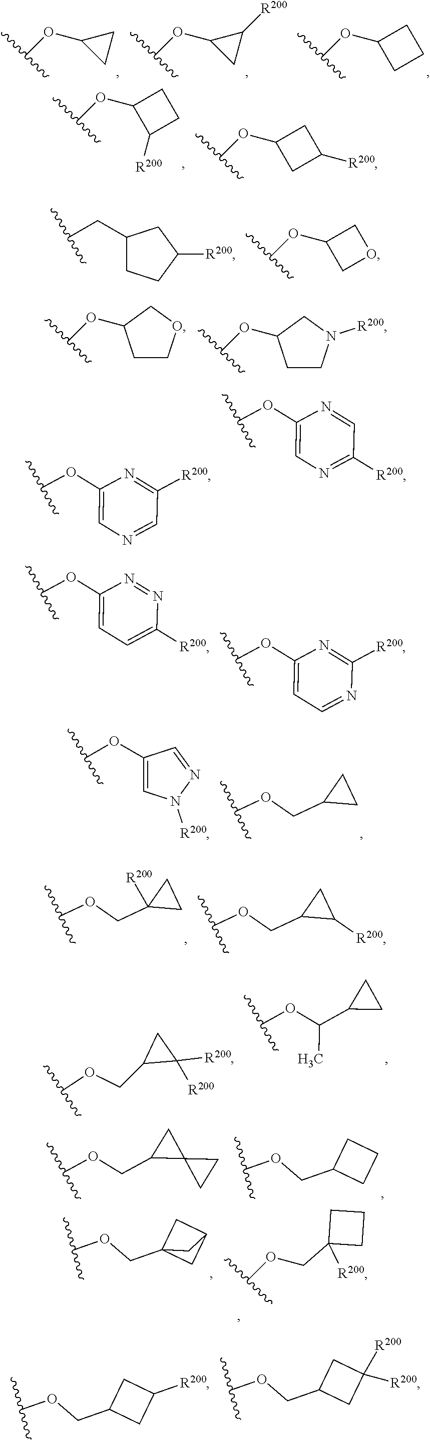

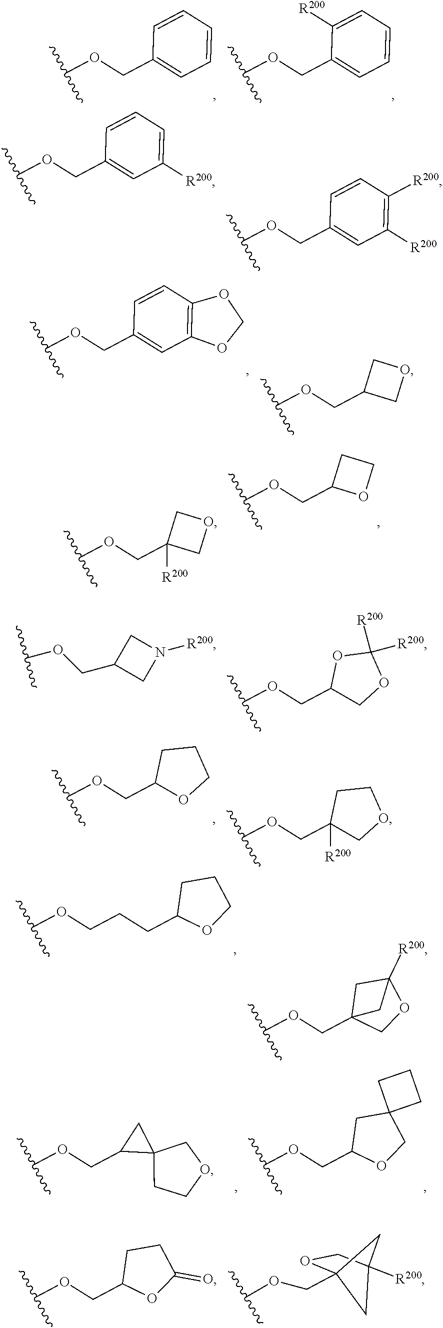

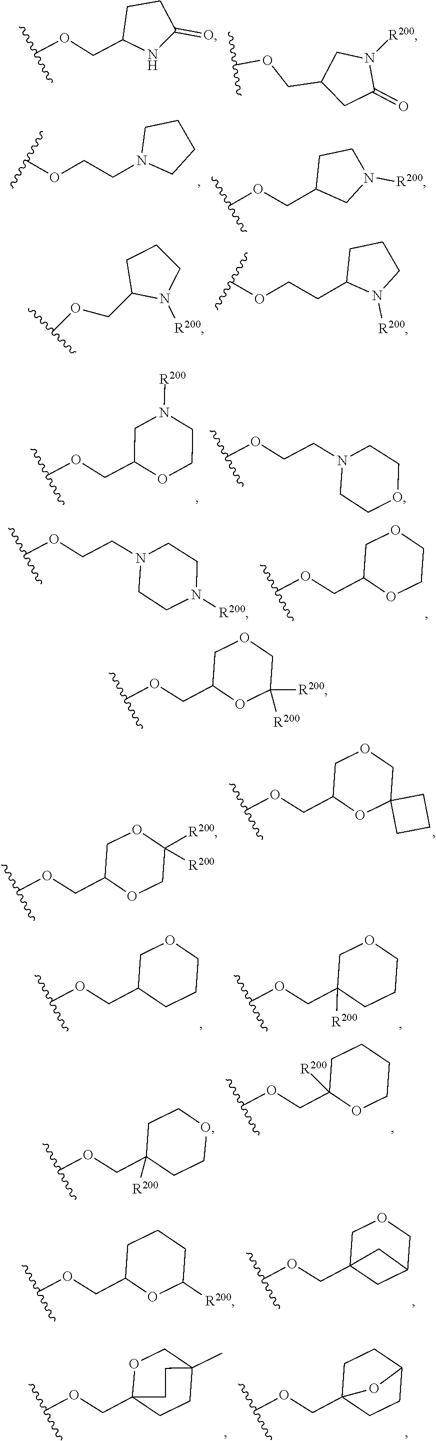

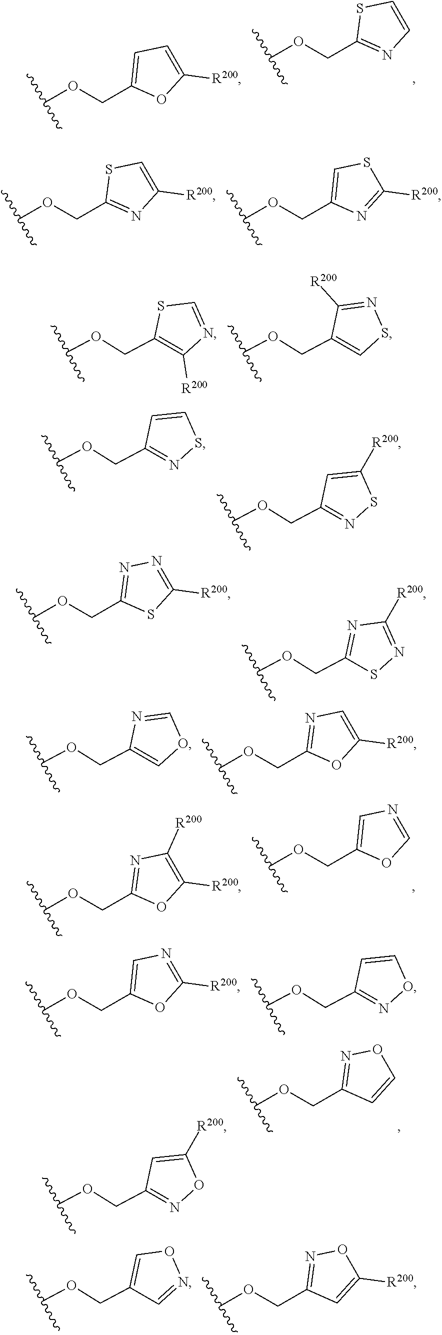

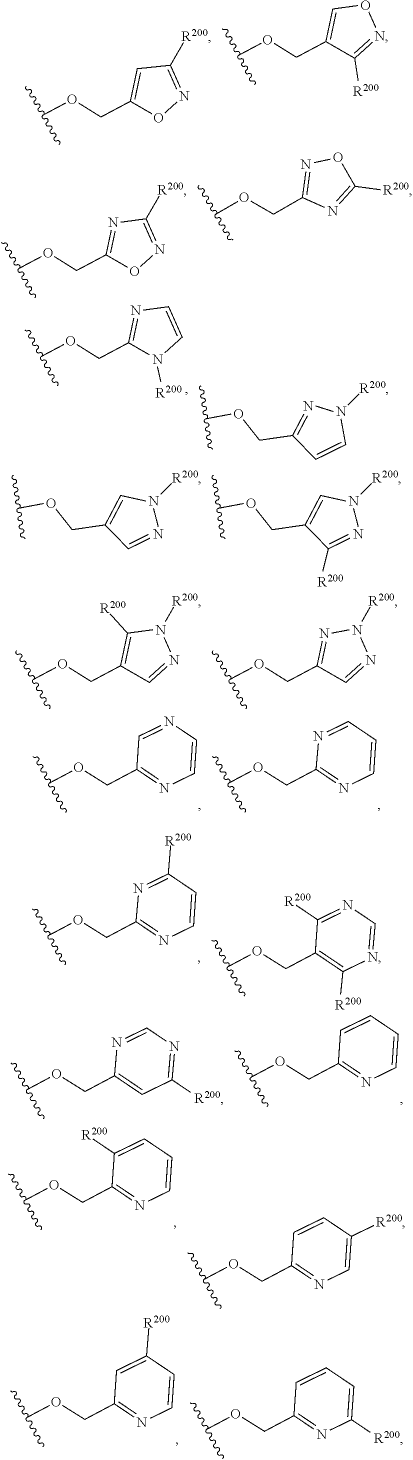

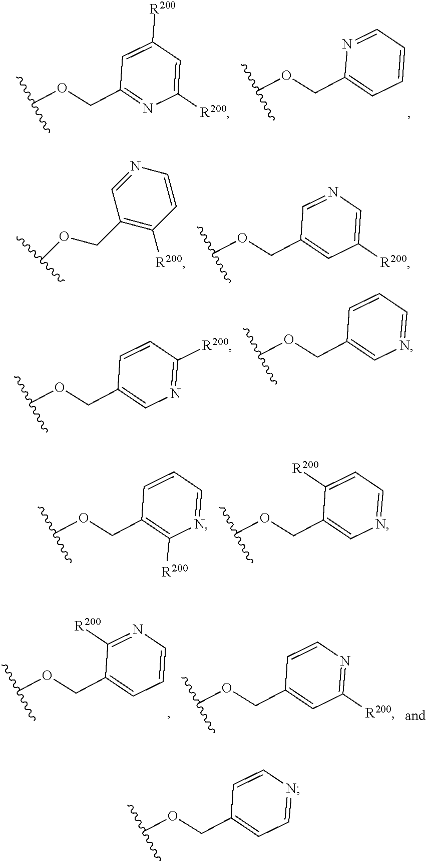

and the remaining variables are as described in the first aspect or the first, second, third, fourth, fifth, sixth, or seventh embodiment.

In an eighteenth embodiment, for the compounds of Formula (I), (II), (II′), (III), (IV), (V), (VI), (VII), (VIII) or (IX), or a pharmaceutically acceptable salt thereof, R²⁰⁰, for each occurrence, is independently selected from F, —CN, —CH₃, —CF₃, —CH₂CH₃, —CH(CH₃)₂, —OCH₃ and cyclopropyl; and the remaining variables are as described in the first aspect or the first, second, third, fourth, fifth, sixth, seventh, eighth, ninth, tenth, eleventh, twelfth, thirteenth, fourteenth, fifteenth, sixteenth, or seventeenth embodiment. In an alternative eighteenth embodiment, for the compounds of Formula (I), (II), (II′), (III), (IV), (V), (VI), (VII), (VIII), or (IX), or a pharmaceutically acceptable salt thereof, R²⁰⁰, for each occurrence, is independently selected from F, —CN, —CH₃, —CH₂F, —CF₃, —CH₂CH₃, —CH(CH₃)₂, —CH₂OCH₃, —OCH₃, cyclobutyl, and cyclopropyl; and the remaining variables are as described in the first aspect or the first, second, third, fourth, fifth, sixth, seventh, eighth, ninth, tenth, eleventh, twelfth, thirteenth, fourteenth, fifteenth, sixteenth, or seventeenth embodiment.

In a nineteenth embodiment, for the compounds of Formula (I), (II), (II′), (III), (IV), (V), (VI), (VII), (VIII) or (IX), or a pharmaceutically acceptable salt thereof, R² is C₃₋₆ cycloalkyl, 4- to 6-membered monocyclic heterocyclyl, 7- to 10-membered bicyclic heterocyclyl or 5- to 6-membered monocyclic heteroaryl, each of which is optionally substituted with 1 to 2 R²⁰; and the remaining variables are as described in the first aspect or the first, second, third, fourth, fifth, sixth, or seventh embodiment.

In a twentieth embodiment, for the compounds of Formula (I), (II), (II′), (III), (IV), (V), (VI), (VII), (VIII) or (IX), or a pharmaceutically acceptable salt thereof, R² is selected from azetidinyl, cyclopropyl, dioxino[2,3-d]pyridinyl, isoxazolyl, isothiazolyl, morpholinyl, oxaazabicyclo[3.1.1]heptanyl, oxazolyl, pyradazinyl, pyrazolyl, pyridinyl, pyrrolidinyl, pyrazinyl, pyrimidinyl, triazoyl, oxazoyl, isoxazoyl, oxadiazoyl, thiadiazolyl and thiazolyl, each of which is optionally substituted with 1 to 2 R²⁰; and the remaining variables are as described in the first aspect or the first, second, third, fourth, fifth, sixth, or seventh embodiment. In an alternative twentieth embodiment, for the compounds of Formula (I), (II), (II′), (III), (IV), (V), (VI), (VII), (VIII), or (IX), or a pharmaceutically acceptable salt thereof, R² is selected from azetidinyl, cyclopropyl, tetrahydropyranyl, dioxino[2,3-d]pyridinyl, pyridazinonyl, pyrimidinonyl, pyrazinonyl, isoxazolyl, isothiazolyl, morpholinyl, oxaazabicyclo[3.1.1]heptanyl, oxazolyl, pyradazinyl, pyrazolyl, pyridinyl, pyrazinyl, pyridazinyl, triazinyl, pyrimidinyl, triazoyl, imidazolyl, oxazoyl, isoxazoyl, oxadiazoyl, pyrrolidinyl, thiadiazolyl, thiazolyl, 6,7-dihydro-[1,2,4]triazolo[1,5-a]pyrazin-8(5H)-onyl, 6,7-dihydro-4H-pyrazolo[5,1-c][1,4]oxazinyl, 5,6-dihydro-8H-imidazo[2,1-c][1,4]oxazinyl, 6,7-dihydro-5H-pyrazolo[5,1-b][1,3]oxazinyl, 4,5,6,7-tetrahydro-3H-imidazo[4,5-c]pyridinyl, 3,4,6,7-tetrahydropyrano[3,4-d]imidazolyl, 5,6,7,8-tetrahydroimidazo[1,2-a]pyrazinyl, 4,5,6,7-tetrahydropyrazolo[1,5-a]pyrimidinyl, 4,5,6,7-tetrahydropyrazolo[1,5-a]pyrazinyl, 5,6,7,8-tetrahydro-[1,2,4]triazolo[1,5-a]pyrazine, 5,6-dihydro-8H-[1,2,4]triazolo[5,1-c][1,4]oxazinyl, 5,6-dihydro-8H-imidazo[2,1-c][1,4]oxazinyl, imidazo[1,2-a]pyrimidinyl, [1,2,4]triazolo[4,3-a]pyrazinyl, pyrazolo[1,5-a]pyrimidinyl, [1,2,4]triazolo[1,5-a]pyrimidinyl, imidazo[1,2-b]pyridazinyl, 6,7-dihydro-5H-cyclopenta[b]pyridin-5-onyl, furo[3,4-d]pyrimidin-5(7H)-onyl, 5,7-dihydrofuro[3,4-d]pyrimidinyl, 7,8-dihydro-5H-pyrano[4,3-b]pyridinyl, 2,3-dihydro-[1,4]dioxino[2,3-b]pyridinyl, 5,6-dihydro-4H-pyrrolo[3,4-d]thiazolyl, 5,6-dihydro-4H-pyrrolo[1,2-b]pyrazolyl, and 2,3-dihydroimidazo[2,1-b]oxazolyl, each of which is optionally substituted with 1 to 2 R²⁰; and the remaining variables are as described in the first aspect or the first, second, third, fourth, fifth, sixth, or seventh embodiment.

In a twenty-first embodiment, for the compounds of Formula (I), (II), (II′), (III), (IV), (V), (VI), (VII), (VIII) or (IX), or a pharmaceutically acceptable salt thereof, R² is selected from

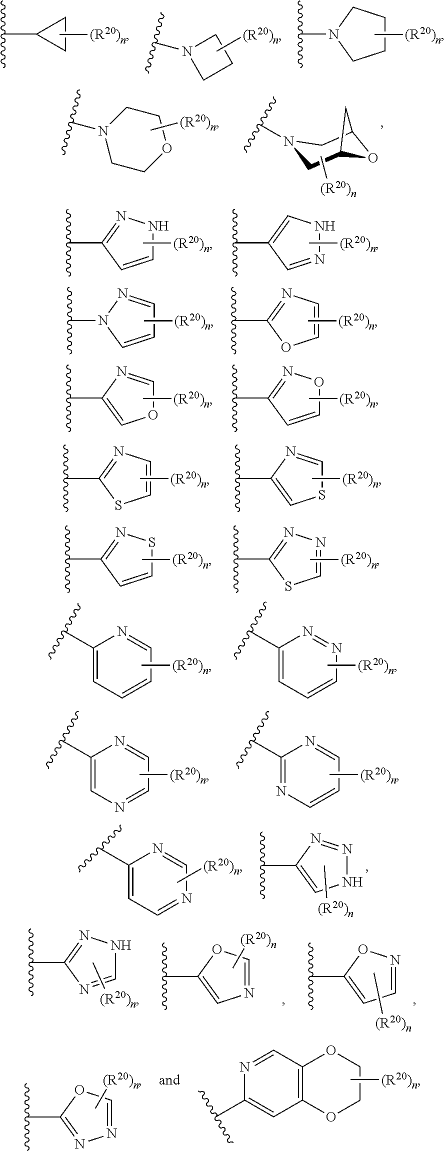

wherein n is 0, 1 or 2; and the remaining variables are as described in the first aspect or the first, second, third, fourth, fifth, sixth, or seventh embodiment. In an alternative twenty-first embodiment, for the compounds of Formula (I), (II), (II′), (III), (IV), (V), (VI), (VII), (VIII), or (IX), or a pharmaceutically acceptable salt thereof, R² is selected from

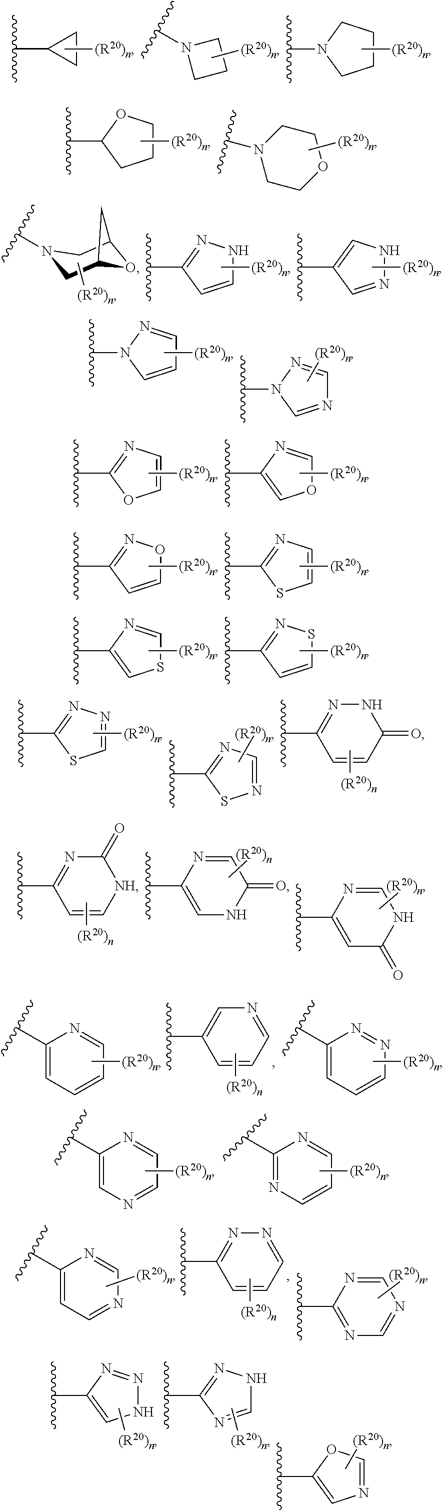

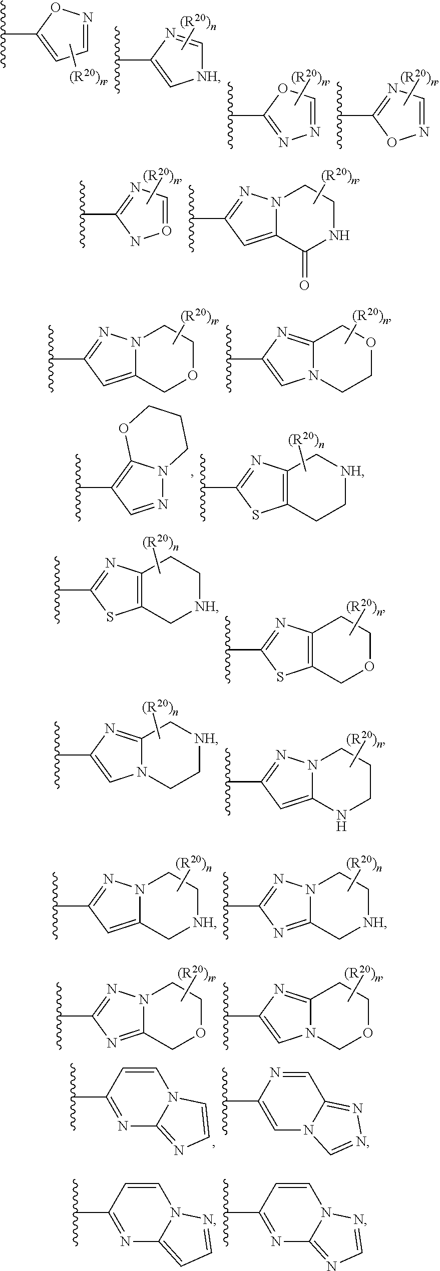

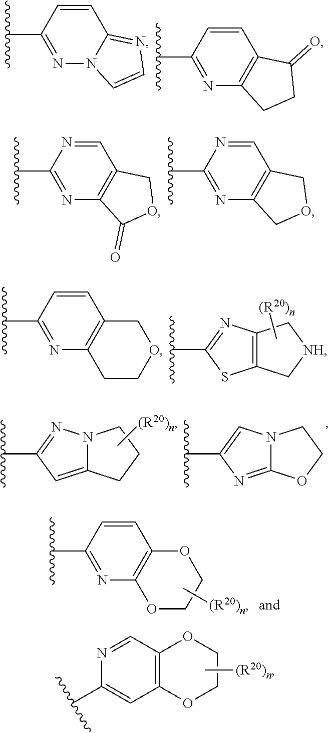

wherein n is 0, 1 or 2; and the remaining variables are as described in the first aspect or the first, second, third, fourth, fifth, sixth, or seventh embodiment.

In a twenty-second embodiment, for the compounds of Formula (I), (II), (II′), (III), (IV), (V), (VI), (VII), (VIII) or (IX), or a pharmaceutically acceptable salt thereof, R²⁰, for each occurrence, is independently halo, —CN, C₁₋₃alkyl, C₁₋₄₃haloalkyl, C₁₋₃ alkoxy or C₃₋₆cycloalkyl; and the remaining variables are as described in the first aspect or the first, second, third, fourth, fifth, sixth, seventh, nineteenth, twentieth, or twenty-first embodiment. In an alternative twenty-second embodiment, for the compounds of Formula (I), (II), (II′), (III), (IV), (V), (VI), (VII), (VIII), or (IX), or a pharmaceutically acceptable salt thereof, R²⁰ for each occurrence, is independently halo, —CN, C₁₋₄alkyl, C₁₋₄haloalkyl, OR^{20c}, —N(R^{20b})₂, —C(O)C₁₋₃alkyl, —SO₂C₁₋₃alkyl, P(O)(C₁₋₃alkyl)₂, C₃₋₆cycloalkyl, or 5- to 10-membered monocyclic or bicyclic heterocyclyl, wherein the C₁₋₄alkyl represented by R²⁰ is optionally substituted by —CN, OH, —N(R^{20b})₂, C₁₋₃alkoxy, C₁₋₃haloalkoxy, C₃₋₆cycloalkyl, and 5- to 10-membered monocyclic or bicyclic heterocyclyl optionally substituted by C₁₋₄alkyl, R^{20c} is H, C₁₋₄alkyl, C₁₋₄haloalkyl, or 4-membered monocyclic heterocyclyl, wherein the C₁₋₄alkyl is optionally substituted by C₁₋₃alkoxy; wherein the 5- to 10-membered monocyclic or bicyclic heterocyclyl represented by R²⁰ is optionally substituted with C₁₋₄alkyl or C₁₋₃alkoxy; each R^{20b} is, independently, H or C₁₋₄alkyl optionally substituted by C₁₋₃alkoxy; and the remaining variables are as described in the first aspect or the first, second, third, fourth, fifth, sixth, seventh, nineteenth, twentieth, or twenty-first embodiment.

In a twenty-third embodiment, for the compounds of Formula (I), (II), (II′), (III), (IV), (V), (VI), (VII), (VIII) or (IX), or a pharmaceutically acceptable salt thereof, R²⁰, for each occurrence, is independently selected from F, —CN, —OCH₃, —CH₃, —CIF₂, cyclopropyl and cyclobutyl; and the remaining variables are as described in the twenty-second embodiment.

In an alternative twenty-third embodiment, for the compounds of Formula (I), (II), (II′), (III), (IV), (V), (VI), (VII), (VIII), or (IX), or a pharmaceutically acceptable salt thereof, R²⁰, for each occurrence, is independently selected from —F, —Cl, —Br, —CN, —OH, —OCH₃, —OCHF₂, —OCH₂CH₃, —OCH(CH₃)₂, —OCH₂CH₂OCH₃, —CH₃, —CD₃, —CHF₂, —CH₂CH₃, —CH(CH₃)₂, —CF(CH₃)₂, —C(CH₃)₃, —CF₂CH₃, —CHFCH₃, —CH₂CH₂CH₃, —CH(CH₃)OH, —CH(CH₃)OCH₃, —CH₂CN, —CH₂N(CH₃)₂, —CH(CH₃)N(CH₃)₂, —CH₂CH₂OCH₃, —CH₂OCH₃, —CH₂OCHF₂, —CH₂N(CH₃)₂, —CH₂C(OH)(CH₃)₂, —CH₂C(OCH₃)(CH₃)₂, —CH₂CH₂OCH₂CH₃, —C(CH₃)₂OH, —C(CH₃)₂OCH₃, —C(CH₃)₂CN, —C(CH₃)₂N(CH₃)₂, —NHCH₃, —N(CH₃)₂, —NHCH(CH₃)₂, —NHCH₂CH₂OCH₃, —CH₂N(CH₃)CH₂CH₂OCH₃, —N(CH₃)CH₂CH₂OCH₃, —C(O)CH₃, SO₂CH₃, —SO₂CH₂CH₃, P(O)(CH₃)₂,

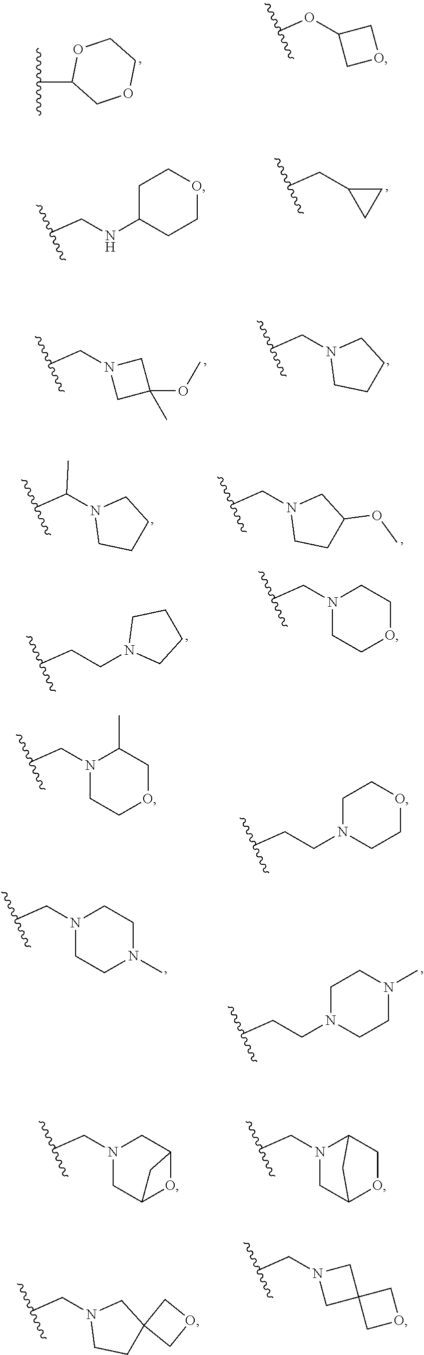

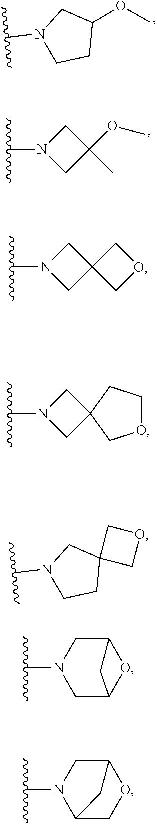

cyclopropyl, cyclobutyl, tetrahydrofuranyl, tetrahydropyranyl, azetidinyl, pyrrolidinyl, N-methylpiperazinyl, N-methylmorpholinyl, and morpholinyl; and the remaining variables are as described in the twenty-second embodiment. In some embodiments, R²⁰, for each occurrence, is independently selected from F, —CN, —OCH₃, —CH₃, —CHF₂, cyclopropyl and cyclobutyl.

In a twenty-fourth embodiment, for the compounds of Formula (I), (II), (II′), (III), (IV), (V), (VI), (VII), (VIII) or (IX), or a pharmaceutically acceptable salt thereof, R³ is selected from H, C₁₋₄alkyl, C₁₋₃haloalkyl, C₃₋₆cycloalkyl, —OR^{3a}, —N(R^{3b})₂ phenyl, pyridinyl, pyrimidinyl, pyrazoyl, thiazoyl, indazoyl, [1,2,4]triazolo[1,5-a]pyridinyl, imidazo[1,2-a]pyridinyl, and imidazo[1,2-b]pyridazinyl, wherein the phenyl, pyridinyl, pyrimidinyl, pyrazoyl, thiazoyl, indazoyl, [1,2,4]triazolo[1,5-a]pyridinyl, imidazo[1,2-a]pyridinyl, or imidazo[1,2-b]pyridazinyl are each optionally substituted by 1 or 2 R^{3c}; R^{3a} is H, C₁₋₃alkyl or C₃₋₆cycloalkyl, wherein the C₁₋₃alkyl and C₃₋₆cycloalkyl represented by R^{3a} are each optionally substituted with one or two substituents independently selected from halo, —CN, C₁₋₂alkyl, —OH and C₁₋₂alkoxy; each R^{3b}, for each occurrence, is independently H, C₁₋₃alkyl, or C₃₋₅cycloalkyl; each R^{3c} is independently selected from halo, oxo, —CN, —OR^{3a}, —N(R^{3b})₂, C₁₋₄alkyl, C₁₋₄alkyl-R^{3d}, C₁₋₄haloalkyl, —C(O)OR^{3a}, phenyl, cyclopropyl, cyclobutyl, oxetanyl, and morpholinyl; R^{3d} is —C(O)OR^{3a}, —N(R^{3b})₂, —OR^{3a}, cyclopropyl, or morpholinyl; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment. In an alternative twenty-fourth embodiment, for the compounds of Formula (I), (II), (II′), (III), (IV), (V), (VI), (VII), (VIII), or (IX), or a pharmaceutically acceptable salt thereof, R³ is selected from H, halo, C₁₋₄alkyl, C₁₋₃haloalkyl, C₃₋₆cycloalkyl, C₂₋₄alkenyl, C₁₋₃alkyl-C₁₋₃alkoxy, —OR^{3a}, —N(R^{3b})₂, phenyl, pyridinyl, pyrimidinyl, pyrazoyl, thiazoyl, indazoyl, [1,2,4]triazolo[1,5-a]pyridinyl, imidazo[1,2-a]pyridinyl, or imidazo[1,2-b]pyridazinyl, wherein the phenyl, pyridinyl, pyrimidinyl, pyrazoyl, thiazoyl, indazoyl, [1,2,4]triazolo[1,5-a]pyridinyl, imidazo[1,2-a]pyridinyl, or imidazo[1,2-b]pyridazinyl are each optionally substituted by 1 or 2 R^{3c}; R^{3a} is H, C₁₋₃alkyl, 4- to 8-membered monocyclic or bicycle heterocyclyl, or C₃₋₆cycloalkyl, wherein the C₁₋₃alkyl and C₃₋₆cycloalkyl represented by R^{3a} are each optionally substituted with one or two substituents independently selected from halo, —CN, C₁₋₂alkyl, —OH and C₁₋₂alkoxy; each R^{3b}, for each occurrence, is independently H, C₁₋₃ alkyl, or C₃₋₅cycloalkyl; each R^{3c} is independently selected from halo, oxo, —CN, —OR^{3a}, —N(R^{3b})₂, C₁₋₄alkyl, C₁₋₄alkyl-R^{3d}, C₁₋₄haloalkyl, —C(O)OR^{3a}, phenyl, cyclopropyl, cyclobutyl, oxetanyl, or morpholinyl; R^{3d} is —C(O)OR^{3a}, —N(R^{3b})₂, —OR^{3a}, cyclopropyl, or morpholinyl; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-fifth embodiment, for the compounds of Formula (I), (II), (II′). (III), (IV), (V), (VI), (VII), (VIII), (IX), or a pharmaceutically acceptable salt thereof, R³ is selected from H, —CH₃, —CH₂CH₃, —CF₂CH₃, —CH(CH₃)₂, cyclopropyl, —OCH₃, —OCH₂CH₂OCH₃, —NHCH₃,

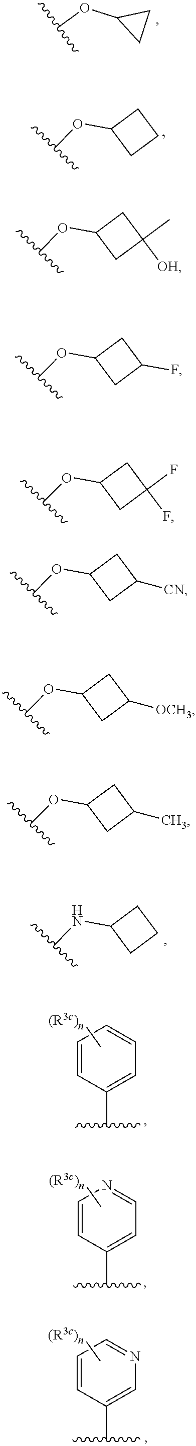

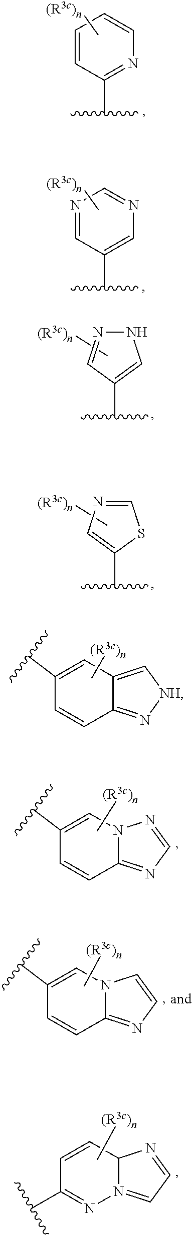

wherein n is 0, 1, or 2; and the remaining variables are as described in the twenty-fourth embodiment. In an alternative twenty-fifth embodiment, for the compounds of Formula (I), (II′), (II), (III), (IV), (V), (VI), (VII), (VIII), or (IX), or a pharmaceutically acceptable salt thereof, R³ is selected from H, —F, —Cl, —CH₃, —CH₂CH₃, —CF₂CH₃, —CF₃, —CH(CH₃)₂, cyclopropyl, —CH═CH₂, —CH₂OCH₃, —OCH₃, —OCH₂CH₃, —OCH(CH₃)₂, —OCH₂CH₂OCH₃, —OCH₂CH₂CH₂OCH₃, —NCHCH₃,

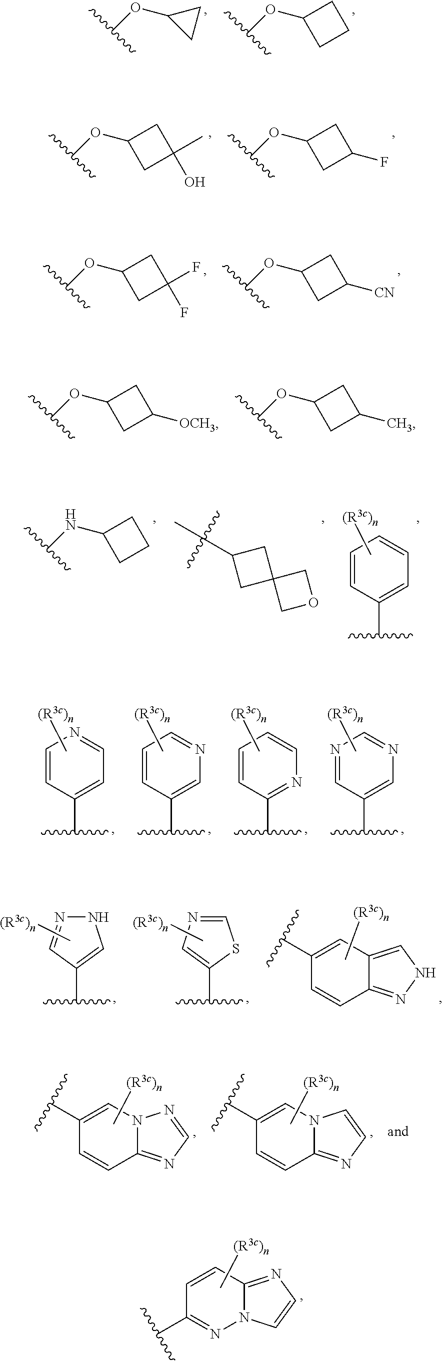

wherein n is 0, 1, or 2; and the remaining variables are as described in the twenty-fourth embodiment.

In a twenty-sixth embodiment, for the compounds of Formula (I), (II), (II′), (III), (IV), (V), (VI), (VII), (VIII) or (IX), or a pharmaceutically acceptable salt thereof, each R^{3c} is individually selected from —CN, F, —OCH₃, —CH₃, —CH₂CH₃, —CH(CH₃)₂, —CH₂CH(CH₃)₂, —CHF₂, —CH₂CF₃, —CF₃, —CD₃, —CH₂CH₂OCH₃, —CH₂-cyclopropyl, —CH₂CH₂-morpholinyl, cyclopropyl, cyclobutyl, —CH₂C(O)OH, —C(O)OC(CH₃)₃, —CH₂CH₂N(CH₃)₂, and morpholinyl; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth or twenty-fifth embodiment. In an alternative twenty-sixth embodiment, for the compounds of Formula (I), (II), (II′), (III), (IV), (V), (VI), (VII), (VIII), or (IX), or a pharmaceutically acceptable salt thereof, each R^{3c} is individually selected from —CN, —F, —Cl, —OCH₃, —CH₃, —CH₂CH₃, —CH(CH₃)₂, —CH₂CH(CH₃)₂, —CHF₂, —CH₂CF₃, —CF₃, —CD₃, —CH₂CH₂OCH₃, —CH₂-cyclopropyl, —CH₂CH₂-morpholinyl, cyclopropyl, cyclobutyl, —CH₂C(O)OH, —C(O)OC(CH₃)₃, —CH₂CH₂N(CH₃)₂, oxetanyl, and morpholinyl; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth or twenty-fifth embodiment.

In a twenty-seventh embodiment, for the compounds of Formula (I), (II), (II′), (III), (IV), (V), (VI), (VII), (VIII) or (IX), or a pharmaceutically acceptable salt thereof, R⁴ is selected from C₁₋₄haloalkyl and C₃₋₆cycloalkyl optionally substituted with 1 to 3 substituents independently selected from halo and C₁₋₃alkyl; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment. In an alternative twenty-seventh embodiment, for the compounds of Formula (I), (II), (II′), (III), (IV), (V), (VI), (VII), (VIII), or (IX), or a pharmaceutically acceptable salt thereof, R⁴ is selected from C₁₋₄haloalkyl, C₁₋₃alkoxy, C₁₋₃alkoxy-C₁₋₃alkoxy, C₁₋₃haloalkoxy, —C₂₋₄alkenyl, C₂₋₄haloalkenyl, 5- to 7 membered monocyclic or bicyclic heterocyclyl, and C₃₋₆cycloalkyl, wherein the 5- to 7 membered monocyclic or bicyclic heterocyclyl, and C₃₋₆cycloalkyl are each optionally substituted with 1 to 3 substituents independently selected from halo, C₁₋₃haloalkyl, C₁₋₃alkyl-C₁₋₃alkoxy, and C₁₋₃alkyl; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment.

In a twenty-eighth embodiment, for the compounds of Formula (I), (II), (II′), (III), (IV), (V), (VI), (VII), (VIII) or (IX), or a pharmaceutically acceptable salt thereof, R⁴ is selected from —CF₂CH₃, —CF₂CFH₂, —CFHCFH₂, —CF₂CH₂CH₃, —CF(CH₃)₂, and

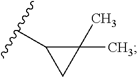 [SMILES: *C1CC1(C)C]

and the remaining variables are as described in the twenty-seventh embodiment. In an alternative twenty-seventh embodiment, for the compounds of Formula (I), (II), (II′), (III), (IV), (V), (VI), (VII), (VIII), or (IX), or a pharmaceutically acceptable salt thereof, R⁴ is selected from —CF₂CH₃, —CF₂CFH₂, —CFHCFH₂, —CF(CH₃)₂, —CF(CH₃)CFH₂, —CH(CH₃)CFH₂, —CF₂CH₂CH₃, —CF(CH₃)₂, —OCH₃, —OCF₂, —OCH₂CH₂OCH₃—CF=CH₂,

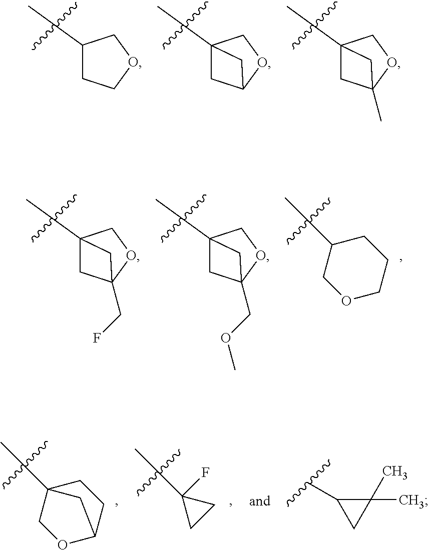

and the remaining variables are as described in the twenty-seventh embodiment.

In a twenty-ninth embodiment, for the compounds of Formula (I), (II), (II′), (III), (IV), (V), (VI), (VII), (VIII) or (IX), or a pharmaceutically acceptable salt thereof, R⁵ is H or 5-membered heteroaryl having 1 to 3 heteroatoms independently selected from nitrogen, oxygen and sulfur, wherein the 5-membered heteroaryl represented by R⁵ is optionally substituted by 1 to 3 R⁵⁰; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, or twenty-eighth embodiment.

In a thirtieth embodiment, for the compounds of Formula (I), (II), (II′), (III), (IV), (V), (VI), (VII), (VIII) or (IX), or a pharmaceutically acceptable salt thereof, R⁵ is H or pyrazolyl optional substituted by 1 to 3 R⁵⁰; and the remaining variables are as described in the fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, or twenty-ninth embodiment.

In some embodiments, for the compounds of Formula (I), (II), (II′), (III), (IV), (V), (VI), (VII), (VIII), or (IX), or a pharmaceutically acceptable salt thereof, R⁶ is H, halo, C₁₋₃ alkoxy, and the remaining variables are as described in the first aspect or any of the embodiments described above. Alternatively, for the compounds of Formula (I), (II), (II′), (III), (IV), (V), (VI), (VII), (VIII), or (IX), or a pharmaceutically acceptable salt thereof, R⁶ is H, —F, or —OCH₃, and the remaining variables are as described in the first aspect or any of the embodiments described above.

In some embodiments, for the compounds of Formula (I), (II), (II′), (III), (IV), (V), (VI), (VII), (VIII), or (IX), or a pharmaceutically acceptable salt thereof, R⁷ is H, and the remaining variables are as described in the first aspect or any of the embodiments described above.

In a thirty-first embodiment, the compound of the present disclosure is represented by Formula (IV-1) or (V-1):

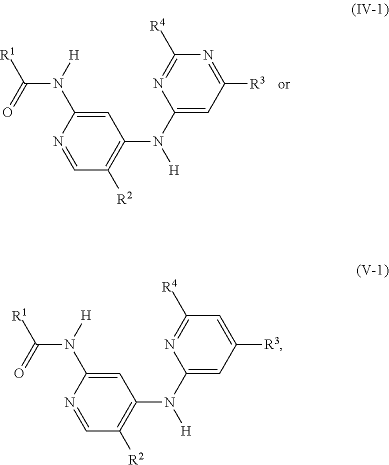

or a pharmaceutically acceptable salt thereof, wherein:
R¹ is C₁₋₃alkyl;
R² is —OR^{2a} or 5-membered monocyclic heteroaryl optionally substituted with C₁₋₃alkyl;
   R^{2a} is C₁₋₄alkyl optionally substituted with R²⁰;
   R²⁰ is C₁₋₃alkoxy or C₃₋₆cycloalkyl optionally substituted with C₁₋₂alkoxy;
R³ is selected from H, —OR^{3a}, C₁₋₃alkyl, C₃₋₆cycloalkyl, and pyrazoyl, wherein the pyrazoyl is optionally substituted by 1 or 2 R^{3c};
   R^{3a} is C₁₋₃alkyl optionally substituted with C₁₋₃alkoxy, or C₃₋₆cycloalkyl optionally substituted with 1 or 2 substituents independently selected from C₁₋₃alkoxy, C₁₋₃alkyl and —OH;
   R^{3c} is C₁₋₃alkyl; and
R⁴ is C₁₋₃haloalkyl; and the remaining variables are as described in the first aspect or the first embodiment.

In a thirty-second embodiment, for the compounds of Formula (I), (IV-1), or (V-1), or a pharmaceutically acceptable salt thereof, R¹ is —CH₃; and the remaining variables are as described in the thirty-first embodiment.

In a thirty-third embodiment, for the compounds of Formula (I), (IV-1), or (V-1), or a pharmaceutically acceptable salt thereof, R² is selected from —OCH₃, —OCD₃, —OCH₂CH₃, —OCD₂CH₃, —OCD₂CD₃, —OCH₂CH₂OCH₃,

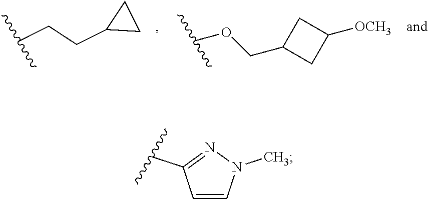 [SMILES: *CCC1CC1 ; *OCC1CC(OC)C1 ; *c1ccn(C)n1]

and the remaining variables are as described in the thirty-first or thirty-second embodiment.

In a thirty-fourth embodiment, for the compounds of Formula (I), (IV-1), or (V-1), or a pharmaceutically acceptable salt thereof, R³ is selected from H, —CH₃, —CH₂CH₃, cyclopropyl, —OCH₃, —OCH₂CH₂OCH₃,

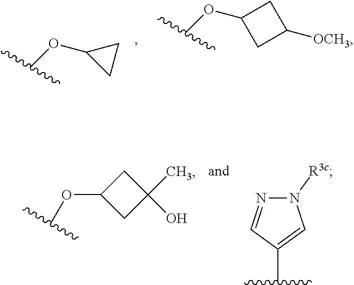

and the remaining variables are as described in the thirty-first, thirty-second, or thirty-third embodiment.

In a thirty-fifth embodiment, for the compounds of Formula (I), (IV-1), or (V-1), or a pharmaceutically acceptable salt thereof, R^{3c} is —CH₃, R⁴ is —CF₂CH₃, —CF₂CFH₂, —CFHCFH₂, —CF₂CH₂CH₃, —CF(CH₃)₂; and the remaining variables are as described in the thirty-first, thirty-second, thirty-third, or thirty-fourth embodiment.

In a thirty-sixth embodiment, the compound of the present disclosure is represented by Formula (III):

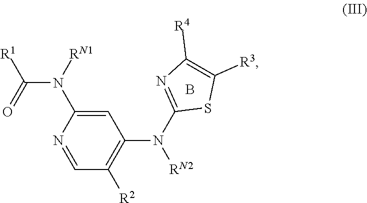

or a pharmaceutically acceptable salt thereof, wherein:
R¹ is C₁₋₆alkyl;
R² is C₁₋₄alkoxy;
R³ is H or C₁₋₆alkyl;
R⁴ is C₁₋₄haloalkyl or 4- to 6-membered monocyclic heterocyclyl having 1 to 2 heteroatoms independently selected from nitrogen and oxygen; and
R^{N1} and R^{N2} are each independently H or C₁₋₃alkyl; and the remaining variables are as described in the first aspect or the first, second, or third embodiment.

In a thirty-seventh embodiment, for the compounds of Formula (I) or (III), or a pharmaceutically acceptable salt thereof, R¹ is —CH₃; and the remaining variables are as described in the thirty-sixth embodiment.

In a thirty-eighth embodiment, for the compounds of Formula (I) or (III), or a pharmaceutically acceptable salt thereof, R² is —OCH₂CH₃ or —OCH₂CH₂OCH₃; and the remaining variables are as described in the thirty-sixth or thirty-seventh embodiment.

In a thirty-ninth embodiment, for the compounds of Formula (I) or (III), or a pharmaceutically acceptable salt thereof, R³ is H or —CH₃; and the remaining variables are as described in the thirty-sixth, thirty-seventh, or thirty-eighth embodiment.

In a fortieth embodiment, for the compounds of Formula (I) or (III), or a pharmaceutically acceptable salt thereof, R⁴ is C₁₋₃haloalkyl or tetrahydrofuranyl; and the remaining variables are as described in the thirty-sixth, thirty-seventh, thirty-eighth, or thirty-ninth embodiment.

In a forty-first embodiment, for the compounds of Formula (I) or (III), or a pharmaceutically acceptable salt thereof, R⁴ is —CF₂CH₃ or

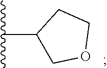 [SMILES: *C1CCOC1]

and the remaining variables are as described in the fortieth embodiment.

In a forty-second embodiment, the compound of the present disclosure is represented by Formula (X):

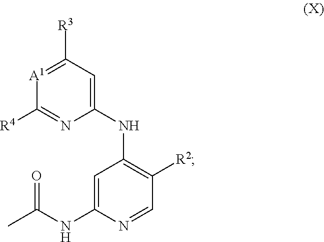

or a pharmaceutically acceptable salt thereof, wherein:
A¹ is N or CH;
R² is —OR^{2a}, 5- or 6-membered monocyclic heteroaryl, or 7- to 10-membered bicyclic heterocyclyl, wherein the 5- or 6-membered monocyclic heteroaryl or 7- to 10-membered bicyclic heterocyclyl are each optionally substituted with one or two R²⁰;
   R^{2a} is C₁₋₃alkyl optionally substituted with C₁₋₃alkoxy;
   R²⁰ is C₁₋₃alkyl optionally substituted by —N(C₁₋₃alkyl)₂;
R³ is H, C₁₋₃alkyl, or —OR³a;
   R^{3a} is C₃₋₄cycloalkyl;
R⁴ is C₁₋₄haloalkyl or 5- to 7-membered bicyclic heterocyclyl; and the remaining variables are as described in the first aspect or first embodiment.

In a forty-third embodiment, for the compounds of Formula (I) or (X), or a pharmaceutically acceptable salt thereof, A¹ is N; and the remaining variables are as described in the forty-second embodiment.

In a forty-fourth embodiment, for the compounds of Formula (I) or (X), or a pharmaceutically acceptable salt thereof, R² is —OCH₃, —OCH₂CH₃, or —OCH₂CH₂OCH₃; and the remaining variables are as described in the forty-second or forty-third embodiment.

In a forty-fifth embodiment, for the compounds of Formula (I) or (X), or a pharmaceutically acceptable salt thereof, R² is pyrazoyl pyridinyl, pyrimidinyl, or 4,5,6,7-tetrahydropyrazolo[1,5-a]pyrazinyl, each of which is optionally substituted by one or two R²⁰; and the remaining variables are as described in the forty-second or forty-third embodiment.

In a forty-sixth embodiment, for the compounds of Formula (I) or (X), or a pharmaceutically acceptable salt thereof, R² is

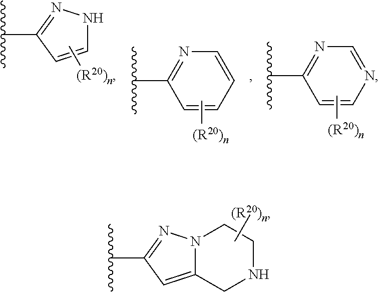

wherein n is 0, 1 or 2; and the remaining variables are as described in the forty-fifth embodiment.

In a forty-seventh embodiment, for the compounds of Formula (I) or (X), or a pharmaceutically acceptable salt thereof, R² is

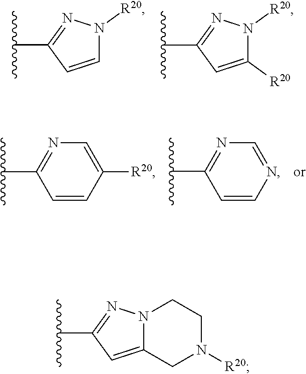

and the remaining variables are as described in the forty-sixth embodiment.

In a forty-eighth embodiment, for the compounds of Formula (I) or (X), or a pharmaceutically acceptable salt thereof, each R²⁰ is, independently, —CH₃, —CH₂CH₃, or —CH₂N(CH₃)₂; and the remaining variables are as described in the forty-second, forty-third, forty-fifth, forty-sixth, or forty-seventh embodiment.

In a forty-ninth embodiment, for the compounds of Formula (I) or (X), or a pharmaceutically acceptable salt thereof, R³ is H, —CH₃, —CH₂CH₃, or —O-cyclopropyl; and the remaining variables are as described in the forty-second, forty-third, forty-fourth, forty-fifth, forty-sixth, forty-seventh, or forty-eighth embodiment.

In a fiftieth embodiment, for the compounds of Formula (I) or (X), or a pharmaceutically acceptable salt thereof, R⁴ is —CF₂CH₃ or

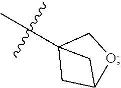 [SMILES: *C12COC(C1)C2]

and the remaining variables are as described in the forty-second, forty-third, forty-fourth, forty-fifth, forty-sixth, forty-seventh, forty-eighth, or forty-ninth embodiment.

In a fifty-first embodiment, the present disclosure provides a compound selected from the group consisting of:
N-(4-((6-(1,1-difluoroethyl)pyridin-2-yl)amino)-5-ethoxypyridin-2-yl)acetamide;
N-(4-((4-(1,1-difluoroethyl)pyridin-2-yl)amino)-5-ethoxypyridin-2-yl)acetamide;
N-(4-((4-(1,1-difluoroethyl)pyrimidin-2-yl)amino)-5-ethoxypyridin-2-yl)acetamide;
N-(4-((2-(1,1-difluoroethyl)pyrimidin-4-yl)amino)-5-ethoxypyridin-2-yl)acetamide;
N-(4-((2-(1,1-difluoroethyl)-6-methoxypyrimidin-4-yl)amino)-5-ethoxypyridin-2-yl)acetamide;
N-(4-((2-(1,1-difluoroethyl)pyrimidin-4-yl)amino)-5-(difluoromethoxy)pyridin-2-yl)acetamide;
N-(5-cyclobutyloxy-4-((2-(1,1-difluoroethyl)pyrimidin-4-yl)amino)pyridin-2-yl)acetamide;
N-(4-((2-(1,1-difluoroethyl)pyrimidin-4-yl)amino)-5-propan-2-yloxypyridin-2-yl)acetamide;
N-(4-((2-(1,1-difluoroethyl)-6-methylpyrimidin-4-yl)amino)-5-ethoxypyridin-2-yl)propanamide;
N-(4-((2-(1,1-difluoroethyl)-6-methylpyrimidin-4-yl)amino)-5-ethoxypyridin-2-yl)acetamide;
N-(5-(cyclopropylmethoxy)-4-((2-(1,1-difluoroethyl)-6-methylpyrimidin-4-yl)amino)pyridin-2-yl)acetamide;
N-(4-((2-(1,1-difluoroethyl)-6-ethylpyrimidin-4-yl)amino)-5-ethoxypyridin-2-yl)acetamide;
N-(4-((6-cyclopropyl-2-(1,1-difluoroethyl)pyrimidin-4-yl)amino)-5-ethoxypyridin-2-yl)acetamide;
N-(4-((6-(1,1-difluoroethyl)-4-(3-methoxycyclobutyl)oxypyridin-2-yl)amino)-5-ethoxypyridin-2-yl)acetamide;
N-(4-((6-(1,1-difluoroethyl)-4-(3-methoxycyclobutyl)oxypyridin-2-yl)amino)-5-ethoxypyridin-2-yl)acetamide;
N-(4-((2-(1,1-difluoroethyl)-6-(3-methoxycyclobutyl)oxypyrimidin-4-yl)amino)-5-ethoxypyridin-2-yl)acetamide;
N-(4-((6-cyclopropyl-2-(1,1-difluoroethyl)pyrimidin-4-yl)amino)-5-methoxypyridin-2-yl)acetamide;
N-(4-((2-(1,1-difluoroethyl)-6-methoxypyrimidin-4-yl)amino)-5-methoxypyridin-2-yl)acetamide;
N-(4-((2-(1,1-difluoroethyl)-6-(3-methoxycyclobutyl)oxypyrimidin-4-yl)amino)-5-methoxypyridin-2-yl)acetamide;
N-(4-((2-(1,1-difluoroethyl)-6-ethylpyrimidin-4-yl)amino)-5-(2-methoxyethoxy)pyridin-2-yl)acetamide;
N-(4-((2-(1,1-difluoroethyl)-6-methoxypyrimidin-4-yl)amino)-5-(2-methoxyethoxy)pyridin-2-yl)acetamide;
N-(4-((2-(1,1-difluoroethyl)-6-methylpyrimidin-4-yl)amino)-5-(2-methoxyethoxy)pyridin-2-yl)acetamide;
N-(4-((2-(1,1-difluoroethyl)-6-methylpyrimidin-4-yl)amino)-5-(2,2,2-trifluoroethoxy)pyridin-2-yl)acetamide;
N-(5-(2,2-difluoroethoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(2-(dimethylamino)ethoxy)pyridin-2-yl)acetamide; formic acid;
N-(4-((2-(1,1-difluoroethyl)-6-methylpyrimidin-4-yl)amino)-5-ethoxypyridin-2-yl)-2-methoxyacetamide;
N-(4-((2-(1,1-difluoroethyl)-6-methylpyrimidin-4-yl)amino)-5-ethoxypyridin-2-yl)-3-methoxypropanamide;
N-(4-((2-(1,1-difluoroethyl)-6-methylpyrimidin-4-yl)amino)-5-ethoxypyridin-2-yl)cyclopropanecarboxamide;
N-(5-cyclopropyl-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-phenylmethoxypyridin-2-yl)acetamide;
N-(5-ethoxy-4-((2-(2-fluoropropan-2-yl)pyrimidin-4-yl)amino)pyridin-2-yl)acetamide;
N-(4-((2-(1,1-difluoroethyl)-6-(2-methoxyethoxy)pyrimidin-4-yl)amino)-5-ethoxypyridin-2-yl)acetamide;
N-(4-((6-(1,1-difluoroethyl)pyrimidin-4-yl)amino)-5-ethoxypyridin-2-yl)acetamide;
N-(4-((2-(1,1-difluoroethyl)-5-fluoropyrimidin-4-yl)amino)-5-ethoxypyridin-2-yl)acetamide;
N-(4-((2-(1,1-difluoroethyl)-5-fluoro-6-methylpyrimidin-4-yl)amino)-5-ethoxypyridin-2-yl)acetamide;
N-(4-((2-(1,1-difluoroethyl)-6-ethylpyrimidin-4-yl)amino)-5-methoxypyridin-2-yl)acetamide;
N-(4-((2-(1,1-difluoroethyl)-6-(2-methoxyethoxy)pyrimidin-4-yl)amino)-5-methoxypyridin-2-yl)acetamide;
N-(5-cyclopropyloxy-4-((2-(1,1-difluoroethyl)pyrimidin-4-yl)amino)pyridin-2-yl)acetamide;
N-(4-((2-(1,1-difluoroethyl)-6-methylpyrimidin-4-yl)amino)-5-propan-2-yloxypyridin-2-yl)acetamide;
N-(4-((2-(1,1-difluoroethyl)-6-methylpyrimidin-4-yl)amino)-5-((3-methoxycyclobutyl)methoxy)pyridin-2-yl)acetamide;
N-(4-((2-(1,1-difluoroethyl)-6-methylpyrimidin-4-yl)amino)-5-methylpyridin-2-yl)acetamide;
N-(4-((2-(1,1-difluoroethyl)-6-methylpyrimidin-4-yl)amino)-5-ethylpyridin-2-yl)acetamide;
N-(4-((2-(1,1-difluoroethyl)-6-methylpyrimidin-4-yl)amino)-5-propylpyridin-2-yl)acetamide;
N-(4-((2-(1,1-difluoroethyl)-6-methylpyrimidin-4-yl)amino)-5-propan-2-ylpyridin-2-yl)acetamide;
N-(5-cyclopropyloxy-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-methoxypyridin-2-yl)acetamide;
N-(4-((2-(1,1-difluoroethyl)-6-methylpyrimidin-4-yl)amino)-5-(methoxymethyl)pyridin-2-yl)acetamide;
N-(5-(methoxymethyl)-4-((6-methoxy-5-(1-methylpyrazol-3-yl)pyridin-2-yl)amino)pyridin-2-yl)acetamide;
N-(4-((2-(1,1-difluoroethyl)-6-methylpyrimidin-4-yl)amino)-5-(1,1,2,2,2-pentadeuterioethoxy)pyridin-2-yl)acetamide;
N-(4-((2-(1,1-difluoroethyl)-6-methoxypyrimidin-4-yl)amino)-5-(1-methylpyrazol-3-yl)pyridin-2-yl)acetamide;
N-(4-((2-(1,1-difluoroethyl)-6-isopropylpyrimidin-4-yl)amino)-5-ethoxypyridin-2-yl)acetamide;
N-(4-((2-(1,1-difluoroethyl)-6-propan-2-ylpyrimidin-4-yl)amino)-5-methoxypyridin-2-yl)acetamide;
N-(4-((2-(1,1-difluoroethyl)-6-(3-hydroxy-3-methylcyclobutyl)oxypyrimidin-4-yl)amino)-5-ethoxypyridin-2-yl)acetamide;
N-(4-((2-(1,1-difluoroethyl)-6-methylpyrimidin-4-yl)-methylamino)-5-ethoxypyridin-2-yl)acetamide;
N-(4-((2-(1,1-difluoroethyl)-6-vinylpyrimidin-4-yl)amino)-5-(2-methoxyethoxy)pyridin-2-yl)acetamide;
N-(4-((6-(1,1-difluoroethyl)pyridin-2-yl)amino)-5-(2-methoxyethoxy)pyridin-2-yl)acetamide;
N-(4-((2-(1,1-difluoroethyl)-6-methylpyrimidin-4-yl)amino)-5-((1-(trifluoromethyl)cyclopropyl)methoxy)pyridin-2-yl)acetamide;
N-(4-((2-(1,1-difluoroethyl)-6-methylpyrimidin-4-yl)amino)-5-((1-methylcyclopropyl)methoxy)pyridin-2-yl)acetamide;
(R)-N-(4-((2-(1,1-difluoroethyl)-6-methylpyrimidin-4-yl)amino)-5-((1-methoxypropan-2-yl)oxy)pyridin-2-yl)acetamide;
(R)-N-(4-((2-(1,1-difluoroethyl)-6-methylpyrimidin-4-yl)amino)-5-(2-methoxypropoxy)pyridin-2-yl)acetamide;
N-(4-((2-(1,1-difluoroethyl)-6-methylpyrimidin-4-yl)amino)-5-(oxetan-3-ylmethoxy)pyridin-2-yl)acetamide;
N-(5-(cyclobutylmethoxy)-4-((2-(1,1-difluoroethyl)-6-methylpyrimidin-4-yl)amino)pyridin-2-yl)acetamide;
N-(4-((6-(1,1-difluoroethyl)-4-methylpyridin-2-yl)amino)-5-(2-methoxyethoxy)pyridin-2-yl)acetamide;
N-(4-((6-(1,1-difluoroethyl)pyridin-2-yl)amino)-5-(2-(pyrrolidin-1-yl)ethoxy)pyridin-2-yl)acetamide;
N-(4-((6-(1,1-difluoroethyl)pyridin-2-yl)amino)-5-(2-(dimethylamino)ethoxy)pyridin-2-yl)acetamide;
N-(4-((6-cyclopropyl-2-(1,1-difluoroethyl)pyrimidin-4-yl)amino)-5-(2-methoxyethoxy)pyridin-2-yl)acetamide;
N-(4-((6-(1,1-difluoroethyl)pyrazin-2-yl)amino)-5-(2-methoxyethoxy)pyridin-2-yl)acetamide;
N-(4-((2-(1,1-difluoroethyl)-6-ethylpyrimidin-4-yl)amino)-5-(2-(difluoromethoxy)ethoxy)pyridin-2-yl)acetamide;
N-(4-((2-(1,1-difluoroethyl)-6-ethylpyrimidin-4-yl)amino)-5-(3-methoxypropoxy)pyridin-2-yl)acetamide;
N-(4-((2-(1,1-difluoroethyl)-6-ethylpyrimidin-4-yl)amino)-5-(2-morpholinoethoxy)pyridin-2-yl)acetamide;
N-(4-((2-(1,1-difluoroethyl)-6-ethylpyrimidin-4-yl)amino)-5-(2-(4-methylpiperazin-1-yl)ethoxy)pyridin-2-yl)acetamide;
(R)-N-(5-(2-methoxyethoxy)-4-((6-(tetrahydrofuran-3-yl)pyridin-2-yl)amino)pyridin-2-yl)acetamide;
(S)—N-(4-((6-(1,2-difluoroethyl)pyridin-2-yl)amino)-5-(2-methoxyethoxy)pyridin-2-yl)acetamide;
(R)-N-(5-((2,2-difluorocyclopropyl)methoxy)-4-((2-(1,1-difluoroethyl)-6-methylpyrimidin-4-yl)amino)pyridin-2-yl)acetamide;
N-(4-((4-(1,1-difluoroethyl)pyrimidin-2-yl)amino)-5-(2-methoxyethoxy)pyridin-2-yl)acetamide;
N-(4-((2-(1,1-difluoroethyl)-6-isopropoxypyrimidin-4-yl)amino)-5-(2-methoxyethoxy)pyridin-2-yl)acetamide;
N-(4-((2-(1,1-difluoroethyl)-6-ethoxypyrimidin-4-yl)amino)-5-(2-methoxyethoxy)pyridin-2-yl)acetamide;
N-(4-((4-(1,1-difluoroethyl)-6-methylpyrimidin-2-yl)amino)-5-(2-methoxyethoxy)pyridin-2-yl)acetamide;
(S)—N-(4-((2-(1,2-difluoroethyl)-6-methylpyrimidin-4-yl)amino)-5-(2-methoxyethoxy)pyridin-2-yl)acetamide;
(S)—N-(4-((4-(1,2-difluoroethyl)-6-methylpyrimidin-2-yl)amino)-5-(2-methoxyethoxy)pyridin-2-yl)acetamide;
(S)—N-(4-((2-(1,2-difluoropropan-2-yl)-6-methylpyrimidin-4-yl)amino)-5-(2-methoxyethoxy)pyridin-2-yl)acetamide;
(S)—N-(4-((2-(1-fluoropropan-2-yl)-6-methylpyrimidin-4-yl)amino)-5-(2-methoxyethoxy)pyridin-2-yl)acetamide;
(R)-N-(5-(1-cyclopropylethoxy)-4-((2-(1,1-difluoroethyl)-6-methylpyrimidin-4-yl)amino)pyridin-2-yl)acetamide;
N-(5-((1-cyanocyclopropyl)methoxy)-4-((2-(1,1-difluoroethyl)-6-methylpyrimidin-4-yl)amino)pyridin-2-yl)acetamide;
(R)-N-(4-((2-(1,1-difluoroethyl)-6-methylpyrimidin-4-yl)amino)-5-(oxetan-2-ylmethoxy)pyridin-2-yl)acetamide;
N-(4-((2-(1,1-difluoroethyl)-6-methylpyrimidin-4-yl)amino)-5-(oxetan-3-yloxy)pyridin-2-yl)acetamide;
methyl (4-((2-(1,1-difluoroethyl)-6-ethylpyrimidin-4-yl)amino)-5-(2-methoxyethoxy)pyridin-2-yl)carbamate;
N-(4-((2-(1,1-difluoroethyl)-6-ethylpyrimidin-4-yl)amino)-5-(2-methoxyethoxy)pyridin-2-yl)propionamide;
(S)—N-(4-((6-(1,2-difluoroethyl)pyrazin-2-yl)amino)-5-(2-methoxyethoxy)pyridin-2-yl)acetamide;
N-(4-((6-(1-fluorovinyl)pyrazin-2-yl)amino)-5-(2-methoxyethoxy)pyridin-2-yl)acetamide;
N-(4-((6-cyclopropoxy-2-(1,1-difluoroethyl)pyrimidin-4-yl)amino)-5-methoxypyridin-2-yl)acetamide;
methyl (4-((2-(1,1-difluoroethyl)-6-ethylpyrimidin-4-yl)amino)-5-(2-(dimethylamino)ethoxy)pyridin-2-yl)carbamate;
N-(4-((6-cyclopropoxy-2-(1,1-difluoroethyl)pyrimidin-4-yl)amino)-5-(trifluoromethoxy)pyridin-2-yl)acetamide;
N-(4-((2-(1,1-difluoroethyl)-6-fluoropyrimidin-4-yl)amino)-5-ethoxypyridin-2-yl)acetamide;
N-(4-((6-(2-oxabicyclo[2.1.1]hexan-4-yl)-4-methylpyridin-2-yl)amino)-5-ethoxypyridin-2-yl)acetamide;
N-(4-((2-(1,1-difluoroethyl)-6-(pyridin-3-yl)pyrimidin-4-yl)amino)-5-ethoxypyridin-2-yl)acetamide;
N-(4-((2-(1,1-difluoroethyl)-6-(1-ethyl-1H-pyrazol-4-yl)pyrimidin-4-yl)amino)-5-methoxypyridin-2-yl)acetamide;
N-(4-((2-(1,1-difluoroethyl)-6-methylpyrimidin-4-yl)amino)-5-((6-methylpyrazin-2-yl)oxy)pyridin-2-yl)acetamide;
N-(4-((2-(1,1-difluoroethyl)-6-methylpyrimidin-4-yl)amino)-5-((1-fluorocyclopropyl)methoxy)pyridin-2-yl)acetamide;
N-(5-(bicyclo[1.1.1]pentan-1-ylmethoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(pyridin-2-ylmethoxy)pyridin-2-yl)acetamide; 2-((6-acetamido-4-((2-(1,1-difluoroethyl)-6-methylpyrimidin-4-yl)amino)pyridin-3-yl)oxy)-N-methylacetamide; 2-((6-acetamido-4-((2-(1,1-difluoroethyl)-6-methylpyrimidin-4-yl)amino)pyridin-3-yl)oxy)-N,N-dimethylacetamide;
N-(4-((2-(1,1-difluoroethyl)-6-methylpyrimidin-4-yl)amino)-5-(3,3-difluoropropoxy)pyridin-2-yl)acetamide;
N-(5-butoxy-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(2-(4-methylpiperazin-1-yl)ethoxy)pyridin-2-yl)acetamide;
N-(4-((2-(1,1-difluoroethyl)-6-methylpyrimidin-4-yl)amino)-5-(3-methoxypropoxy)pyridin-2-yl)acetamide;
N-(4-((2-(1,1-difluoroethyl)-6-methylpyrimidin-4-yl)amino)-5-(thiazol-2-ylmethoxy)pyridin-2-yl)acetamide;
N-(4-((2-(1,1-difluoroethyl)-6-methylpyrimidin-4-yl)amino)-5-((3-ethyloxetan-3-yl)methoxy)pyridin-2-yl)acetamide;
(R)-N-(4-((2-(1,1-difluoroethyl)-6-methylpyrimidin-4-yl)amino)-5-((2,2-dimethyl-1,3-dioxolan-4-yl)methoxy)pyridin-2-yl)acetamide;
N-(4-((2-(1,1-difluoroethyl)-6-methylpyrimidin-4-yl)amino)-5-((3-methylisothiazol-4-yl)methoxy)pyridin-2-yl)acetamide;
N-(4-((2-(1,1-difluoroethyl)-6-methylpyrimidin-4-yl)amino)-5-((4-methylthiazol-2-yl)methoxy)pyridin-2-yl)acetamide;
N-(5-(2-(tert-butoxy)ethoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((2-methoxypyridin-4-yl)methoxy)pyridin-2-yl)acetamide;
N-(4-((2-(1,1-difluoroethyl)-6-methylpyrimidin-4-yl)amino)-5-((5-methylisothiazol-3-yl)methoxy)pyridin-2-yl)acetamide;
N-(4-((2-(1,1-difluoroethyl)-6-methylpyrimidin-4-yl)amino)-5-((6-methylpyridin-3-yl)methoxy)pyridin-2-yl)acetamide;
N-(4-((2-(1,1-difluoroethyl)-6-methylpyrimidin-4-yl)amino)-5-((2-methylthiazol-4-yl)methoxy)pyridin-2-yl)acetamide;
N-(4-((2-(1,1-difluoroethyl)-6-methylpyrimidin-4-yl)amino)-5-((5-methyl-1,3,4-thiadiazol-2-yl)methoxy)pyridin-2-yl)acetamide;
N-(4-((2-(1,1-difluoroethyl)-6-methylpyrimidin-4-yl)amino)-5-(2-fluoro-2-methylpropoxy)pyridin-2-yl)acetamide;
N-(4-((2-(1,1-difluoroethyl)-6-methylpyrimidin-4-yl)amino)-5-((2-fluoropyridin-4-yl)methoxy)pyridin-2-yl)acetamide;
N-(4-((2-(1,1-difluoroethyl)-6-methylpyrimidin-4-yl)amino)-5-((3-methylisoxazol-5-yl)methoxy)pyridin-2-yl)acetamide;
N-(4-((2-(1,1-difluoroethyl)-6-methylpyrimidin-4-yl)amino)-5-(oxazol-5-ylmethoxy)pyridin-2-yl)acetamide;
N-(4-((2-(1,1-difluoroethyl)-6-methylpyrimidin-4-yl)amino)-5-((4,6-dimethylpyridin-2-yl)methoxy)pyridin-2-yl)acetamide;
(S)—N-(4-((2-(1,1-difluoroethyl)-6-methylpyrimidin-4-yl)amino)-5-((2,2-dimethyl-1,3-dioxolan-4-yl)methoxy)pyridin-2-yl)acetamide;
N-(4-((2-(1,1-difluoroethyl)-6-methylpyrimidin-4-yl)amino)-5-((5-fluoropyridin-2-yl)methoxy)pyridin-2-yl)acetamide;
N-(4-((2-(1,1-difluoroethyl)-6-methylpyrimidin-4-yl)amino)-5-(pyrazin-2-ylmethoxy)pyridin-2-yl)acetamide;
N-(4-((2-(1,1-difluoroethyl)-6-methylpyrimidin-4-yl)amino)-5-((5-methoxypyridin-2-yl)methoxy)pyridin-2-yl)acetamide;
N-(4-((2-(1,1-difluoroethyl)-6-methylpyrimidin-4-yl)amino)-5-((4-methoxypyridin-2-yl)methoxy)pyridin-2-yl)acetamide;
N-(5-((4-cyanopyridin-2-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((5-methylpyridin-2-yl)methoxy)pyridin-2-yl)acetamide;
N-(5-((5-cyanopyridin-2-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((6-methoxypyridin-2-yl)methoxy)pyridin-2-yl)acetamide;
N-(4-((2-(1,1-difluoroethyl)-6-methylpyrimidin-4-yl)amino)-5-((4-methylpyridin-2-yl)methoxy)pyridin-2-yl)acetamide;
N-(4-((2-(1,1-difluoroethyl)-6-methylpyrimidin-4-yl)amino)-5-(pyrimidin-2-ylmethoxy)pyridin-2-yl)acetamide;
(R)-N-(5-((1,4-dioxan-2-yl)methoxy)-4-((2-(1,1-difluoroethyl)-6-methylpyrimidin-4-yl)amino)pyridin-2-yl)acetamide;
(S)—N-(4-((2-(1,1-difluoroethyl)-6-methylpyrimidin-4-yl)amino)-5-((tetrahydro-2H-pyran-3-yl)methoxy)pyridin-2-yl)acetamide;
(S)—N-(4-((2-(1,1-difluoroethyl)-6-methylpyrimidin-4-yl)amino)-5-(3-methoxybutoxy)pyridin-2-yl)acetamide;
N-(4-((2-(1,1-difluoroethyl)-6-methylpyrimidin-4-yl)amino)-5-((1,5-dimethyl-1H-pyrazol-4-yl)methoxy)pyridin-2-yl)acetamide;
(R)-N-(4-((6-cyclopropoxy-2-(1,1-difluoroethyl)pyrimidin-4-yl)amino)-5-((tetrahydrofuran-2-yl)methoxy)pyridin-2-yl)acetamide;
N-(4-((6-(1,1-difluoroethyl)pyridin-2-yl)amino)-5-(2-hydroxyethoxy)pyridin-2-yl)acetamide;
N-(4-((2-(2-oxabicyclo[2.1.1]hexan-4-yl)-6-methylpyrimidin-4-yl)amino)-5-(5-methyl-4,5,6,7-tetrahydropyrazolo[1,5-a]pyrazin-2-yl)pyridin-2-yl)acetamide;
N-(4′-((2-(2-oxabicyclo[2.1.1]hexan-4-yl)-6-methylpyrimidin-4-yl)amino)-5-((difluoromethoxy)methyl)-[2,3′-bipyridin]-6′-yl)acetamide;
N-(5-cyclopropyl-4-((2-(1,1-difluoroethyl)-5-methoxypyrimidin-4-yl)amino)pyridin-2-yl)acetamide;
N-(4-((2-(1,1-difluoroethyl)pyrimidin-4-yl)amino)-5-(2,2-dimethyl-2,3-dihydro-[1,4]dioxino[2,3-b]pyridin-6-yl)pyridin-2-yl)acetamide;
N-(5-cyano-4′-((2-(1,1-difluoroethyl)-6-methylpyrimidin-4-yl)amino)-[2,3′-bipyridin]-6′-yl)acetamide;
N-(5-cyano-4′-((2-(1,1-difluoroethyl)pyrimidin-4-yl)amino)-[2,3′-bipyridin]-6′-yl)acetamide;
N-(4-((6-(1,1-difluoroethyl)pyridin-2-yl)amino)-5-(1-methyl-1H-pyrazol-3-yl)pyridin-2-yl)acetamide;
N-(4-((6-(1,1-difluoroethyl)-4-((1r,3r)-3-methoxycyclobutoxy)pyridin-2-yl)amino)-5-(1-methyl-1H-pyrazol-3-yl)pyridin-2-yl)acetamide;
(S)—N-(4-((6-(1,2-difluoroethyl)pyridin-2-yl)amino)-5-(1-methyl-1H-pyrazol-3-yl)pyridin-2-yl)acetamide;
N-(4′-((2-(1,1-difluoroethyl)-6-methylpyrimidin-4-yl)amino)-6-(difluoromethoxy)-[2,3′-bipyridin]-6′-yl)acetamide;
N-(4-((2-(1,1-difluoroethyl)-6-methylpyrimidin-4-yl)amino)-5-(3-(dimethylamino)propyl)pyridin-2-yl)acetamide;
N-(4-((2-(1,1-difluoroethyl)-6-methylpyrimidin-4-yl)amino)-5-(3-methoxypropyl)pyridin-2-yl)acetamide;
N-(5-(cyclopropyldifluoromethyl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(1,1-difluoropropyl)pyridin-2-yl)acetamide;
N-(4-((2-(1,1-difluoroethyl)-6-methylpyrimidin-4-yl)amino)-5-(6-methoxy-2-methylpyrimidin-4-yl)pyridin-2-yl)acetamide;
N-(4-((2-(1,1-difluoroethyl)-6-methylpyrimidin-4-yl)amino)-5-(2-methoxy-6-methylpyrimidin-4-yl)pyridin-2-yl)acetamide;
N-(4-((2-(1,1-difluoroethyl)-6-methylpyrimidin-4-yl)amino)-5-(1-methyl-1H-1,2,4-triazol-3-yl)pyridin-2-yl)acetamide;
N-(4-((6-(1,1-difluoroethyl)pyridin-2-yl)amino)-5-(1-methyl-6-oxo-1,6-dihydropyridazin-3-yl)pyridin-2-yl)acetamide;
N-(4-((6-(1,1-difluoroethyl)pyridin-2-yl)amino)-5-(4-methyl-5-oxo-4,5-dihydropyrazin-2-yl)pyridin-2-yl)acetamide;
N-(4-((6-(1,1-difluoroethyl)pyridin-2-yl)amino)-5-(1-methyl-2-oxo-1,2-dihydropyrimidin-4-yl)pyridin-2-yl)acetamide;
N-(4-((6-(1,1-difluoroethyl)pyridin-2-yl)amino)-5-(1-methyl-6-oxo-1,6-dihydropyrimidin-4-yl)pyridin-2-yl)acetamide;
N-(4-((6-(1,1-difluoroethyl)pyridin-2-yl)amino)-5-(6-methoxypyrimidin-4-yl)pyridin-2-yl)acetamide;
N-(4-((2-(1,1-difluoroethyl)pyrimidin-4-yl)amino)-5-(6-methoxy-2-methylpyrimidin-4-yl)pyridin-2-yl)acetamide;
N-(4-((2-(1,1-difluoroethyl)pyrimidin-4-yl)amino)-5-(2-methoxy-6-methylpyrimidin-4-yl)pyridin-2-yl)acetamide;
N-(4-((2-(1,1-difluoroethyl)pyrimidin-4-yl)amino)-5-(1-methyl-1H-1,2,4-triazol-3-yl)pyridin-2-yl)acetamide;
N-(4-((2-(1,1-difluoroethyl)-6-ethylpyrimidin-4-yl)amino)-5-(1-methyl-1H-1,2,4-triazol-3-yl)pyridin-2-yl)acetamide;
N-(4-((2-(1,1-difluoroethyl)-6-ethylpyrimidin-4-yl)amino)-5-(5-methyl-1,3,4-thiadiazol-2-yl)pyridin-2-yl)acetamide;
N-(4-((2-(1,1-difluoroethyl)-6-ethylpyrimidin-4-yl)amino)-5-(5-methyl-1,3,4-oxadiazol-2-yl)pyridin-2-yl)acetamide;
N-(4-((2-(1,1-difluoroethyl)-6-ethylpyrimidin-4-yl)amino)-5-(3-methyl-1,2,4-thiadiazol-5-yl)pyridin-2-yl)acetamide;
N-(4-((2-(1,1-difluoroethyl)-6-ethylpyrimidin-4-yl)amino)-5-(5-methyl-1,2,4-oxadiazol-3-yl)pyridin-2-yl)acetamide;
N-(4-((6-(difluoromethoxy)pyridin-2-yl)amino)-5-(pyrimidin-4-yl)pyridin-2-yl)acetamide;
N-(4-((6-(1,1-difluoroethyl)pyridin-2-yl)amino)-5-(2-methylpyrimidin-4-yl)pyridin-2-yl)acetamide;
N-(4-((2-(1,1-difluoroethyl)pyrimidin-4-yl)amino)-5-(2-methylpyrimidin-4-yl)pyridin-2-yl)acetamide;
N-(4-((2-(1,1-difluoroethyl)-6-methylpyrimidin-4-yl)amino)-5-(2-methylpyrimidin-4-yl)pyridin-2-yl)acetamide;
N-(4-((2-(2-fluoropropan-2-yl)pyrimidin-4-yl)amino)-5-(2-methylpyrimidin-4-yl)pyridin-2-yl)acetamide;
N-(4-((2-(2-fluoropropan-2-yl)-6-methylpyrimidin-4-yl)amino)-5-(2-methylpyrimidin-4-yl)pyridin-2-yl)acetamide;
N-(4-((6-(1,1-difluoroethyl)pyridin-2-yl)amino)-5-(2-methoxypyrimidin-4-yl)pyridin-2-yl)acetamide;
N-(4-((2-(1,1-difluoroethyl)pyrimidin-4-yl)amino)-5-(2-methoxypyrimidin-4-yl)pyridin-2-yl)acetamide;
N-(4-((2-(1,1-difluoroethyl)-6-methylpyrimidin-4-yl)amino)-5-(2-methoxypyrimidin-4-yl)pyridin-2-yl)acetamide;
N-(4-((2-(2-fluoropropan-2-yl)pyrimidin-4-yl)amino)-5-(2-methoxypyrimidin-4-yl)pyridin-2-yl)acetamide;
N-(4-((2-(2-fluoropropan-2-yl)-6-methylpyrimidin-4-yl)amino)-5-(2-methoxypyrimidin-4-yl)pyridin-2-yl)acetamide;
N-(4-((2-(1,1-difluoroethyl)-6-methylpyrimidin-4-yl)amino)-5-(5-fluoropyrimidin-2-yl)pyridin-2-yl)acetamide;
N-(4-((2-(1,1-difluoroethyl)-6-ethylpyrimidin-4-yl)amino)-5-(5-fluoropyrimidin-2-yl)pyridin-2-yl)acetamide;
N-(4-((2-(1,1-difluoroethyl)-6-isopropylpyrimidin-4-yl)amino)-5-(5-fluoropyrimidin-2-yl)pyridin-2-yl)acetamide;
N-(4-((2-(1,1-difluoroethyl)pyrimidin-4-yl)amino)-5-(5-fluoropyrimidin-2-yl)pyridin-2-yl)acetamide;
N-(4-((6-(1,1-difluoroethyl)pyridin-2-yl)amino)-5-(5-fluoropyrimidin-2-yl)pyridin-2-yl)acetamide;
N-(4-((2-(1,1-difluoroethyl)pyridin-4-yl)amino)-5-(5-fluoropyrimidin-2-yl)pyridin-2-yl)acetamide;
N-(4-((6-(1,1-difluoroethyl)pyrazin-2-yl)amino)-5-(5-fluoropyrimidin-2-yl)pyridin-2-yl)acetamide;
N-(4-((6-(2-fluoropropan-2-yl)pyrazin-2-yl)amino)-5-(5-fluoropyrimidin-2-yl)pyridin-2-yl)acetamide;
N-(4-((6-(1,1-difluoroethyl)-4-methylpyridin-2-yl)amino)-5-(5-fluoropyrimidin-2-yl)pyridin-2-yl)acetamide;
N-(4-((6-(1,1-difluoroethyl)-4-methoxypyridin-2-yl)amino)-5-(5-fluoropyrimidin-2-yl)pyridin-2-yl)acetamide;
N-(4-((2-(1,1-difluoroethyl)-6-methoxypyrimidin-4-yl)amino)-5-(5-fluoropyrimidin-2-yl)pyridin-2-yl)acetamide;
N-(4-((2-(1,1-difluoroethyl)pyrimidin-4-yl)amino)-5-(6-methoxypyrimidin-4-yl)pyridin-2-yl)acetamide;
N-(4-((2-(1,1-difluoroethyl)-6-methylpyrimidin-4-yl)amino)-5-(6-methoxypyrimidin-4-yl)pyridin-2-yl)acetamide;
N-(4-((2-(2-fluoropropan-2-yl)pyrimidin-4-yl)amino)-5-(6-methoxypyrimidin-4-yl)pyridin-2-yl)acetamide;
N-(4-((2-(2-fluoropropan-2-yl)-6-methylpyrimidin-4-yl)amino)-5-(6-methoxypyrimidin-4-yl)pyridin-2-yl)acetamide;
N-(4-((2-(1,1-difluoroethyl)-6-methylpyrimidin-4-yl)amino)-5-(6-methylpyrimidin-4-yl)pyridin-2-yl)acetamide;
N-(4-((2-(2-fluoropropan-2-yl)pyrimidin-4-yl)amino)-5-(6-methylpyrimidin-4-yl)pyridin-2-yl)acetamide;
N-(4-((2-(1,1-difluoroethyl)pyridin-4-yl)amino)-5-(6-methylpyrimidin-4-yl)pyridin-2-yl)acetamide;
N-(4-((2-(2-fluoropropan-2-yl)-6-methylpyrimidin-4-yl)amino)-5-(6-methylpyrimidin-4-yl)pyridin-2-yl)acetamide;
N-(4-((2-(1,1-difluoroethyl)pyrimidin-4-yl)amino)-5-(6-methylpyrimidin-4-yl)pyridin-2-yl)acetamide;
N-(4-((2-(1,1-difluoroethyl)pyrimidin-4-yl)amino)-5-(1-methyl-1H-pyrazol-3-yl)pyridin-2-yl)acetamide;
N-(4-((4-(1,1-difluoroethyl)pyrimidin-2-yl)amino)-5-(1-methyl-1H-pyrazol-3-yl)pyridin-2-yl)acetamide;
N-(4-((2-(1,1-difluoroethyl)-6-ethylpyrimidin-4-yl)amino)-5-(1-methyl-1H-pyrazol-3-yl)pyridin-2-yl)acetamide;
N-(4-((2-(1,1-difluoroethyl)-6-isopropylpyrimidin-4-yl)amino)-5-(1-methyl-1H-pyrazol-3-yl)pyridin-2-yl)acetamide;
N-(4-((6-cyclopropyl-2-(1,1-difluoroethyl)pyrimidin-4-yl)amino)-5-(1-methyl-1H-pyrazol-3-yl)pyridin-2-yl)acetamide;
N-(4-((2-(1,1-difluoroethyl)-6-(2-methoxyethoxy)pyrimidin-4-yl)amino)-5-(1-methyl-1H-pyrazol-3-yl)pyridin-2-yl)acetamide;
(R)-N-(5-(1-methyl-1H-pyrazol-3-yl)-4-((6-(tetrahydrofuran-3-yl)pyridin-2-yl)amino)pyridin-2-yl)acetamide;
N-(4-((6-(1,1-difluoroethyl)-4-methoxypyridin-2-yl)amino)-5-(1-methyl-1H-pyrazol-3-yl)pyridin-2-yl)acetamide;
N-(4-((6-(1,1-difluoroethyl)-4-(2-methoxyethoxy)pyridin-2-yl)amino)-5-(1-methyl-1H-pyrazol-3-yl)pyridin-2-yl)acetamide;
N-(4-((6-(1,1-difluoroethyl)-4-((1s,3s)-3-methoxycyclobutoxy)pyridin-2-yl)amino)-5-(1-methyl-1H-pyrazol-3-yl)pyridin-2-yl)acetamide;
N-(4-((6-(1,1-difluoroethyl)pyrazin-2-yl)amino)-5-(1-methyl-1H-pyrazol-3-yl)pyridin-2-yl)acetamide;
N-(4-((2-(1,1-difluoroethyl)-6-methylpyrimidin-4-yl)amino)-5-(6-methoxypyridazin-3-yl)pyridin-2-yl)acetamide-2,2,2-d3;
N-(4-((2-(1,1-difluoroethyl)pyrimidin-4-yl)amino)-5-(6-methoxypyridazin-3-yl)pyridin-2-yl)acetamide;
N-(4-((6-(2-fluoropropan-2-yl)pyridin-2-yl)amino)-5-(5-fluoropyrimidin-2-yl)pyridin-2-yl)acetamide;
N-(4-((2-(2-fluoropropan-2-yl)pyridin-4-yl)amino)-5-(5-fluoropyrimidin-2-yl)pyridin-2-yl)acetamide;
N-(4-((2-(1,1-difluoroethyl)pyridin-4-yl)amino)-5-(6-methoxypyrimidin-4-yl)pyridin-2-yl)acetamide;
N-(4-((2-(1,1-difluoroethyl)-6-methylpyrimidin-4-yl)amino)-5-(1-methyl-1H-pyrazol-3-yl)pyridin-2-yl)acetamide;
N-(4-((2-(1,1-difluoroethyl)-6-methylpyrimidin-4-yl)amino)-5-(6-methylpyridazin-3-yl)pyridin-2-yl)acetamide;
N-(4-((2-(1,1-difluoroethyl)-6-ethylpyrimidin-4-yl)amino)-5-(6-methoxypyridazin-3-yl)pyridin-2-yl)acetamide;
N-(4-((2-(1,1-difluoroethyl)-6-ethylpyrimidin-4-yl)amino)-5-(6-oxo-1,6-dihydropyridazin-3-yl)pyridin-2-yl)acetamide;
N-(5-(5-acetyl-4,5,6,7-tetrahydropyrazolo[1,5-a]pyrazin-2-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(4-oxo-4,5,6,7-tetrahydropyrazolo[1,5-a]pyrazin-2-yl)pyridin-2-yl)acetamide;
(R)-N-(4-((2-(1,1-difluoroethyl)-6-methylpyrimidin-4-yl)amino)-5-(7-hydroxy-5-methyl-4,5,6,7-tetrahydropyrazolo[1,5-a]pyrazin-2-yl)pyridin-2-yl)acetamide;
N-(4-((2-(1,1-difluoroethyl)-6-methylpyrimidin-4-yl)amino)-5-(5-(dimethylamino)pyrazin-2-yl)pyridin-2-yl)acetamide;
N-(4-((2-(1,1-difluoroethyl)-6-methylpyrimidin-4-yl)amino)-5-(5-methyl-4,5,6,7-tetrahydropyrazolo[1,5-a]pyrazin-2-yl)pyridin-2-yl)acetamide;
N-(4-((2-(1,1-difluoroethyl)-6-methylpyrimidin-4-yl)amino)-5-(2-methyl-2H-1,2,3-triazol-4-yl)pyridin-2-yl)acetamide;
N-(4-((2-(1,1-difluoroethyl)-6-methylpyrimidin-4-yl)amino)-5-(1-(2-hydroxy-2-methylpropyl)-1H-pyrazol-3-yl)pyridin-2-yl)acetamide;
N-(4-((2-(1,1-difluoroethyl)-6-methylpyrimidin-4-yl)amino)-5-(1-(2-ethoxyethyl)-1H-pyrazol-3-yl)pyridin-2-yl)acetamide;
N-(4-((2-(1,1-difluoroethyl)-6-methylpyrimidin-4-yl)amino)-5-(1-(2-methoxy-2-methylpropyl)-1H-pyrazol-3-yl)pyridin-2-yl)acetamide;
N-(4-((2-(1,1-difluoroethyl)-6-methylpyrimidin-4-yl)amino)-5-(5-(methoxymethyl)thiazol-2-yl)pyridin-2-yl)acetamide;
N-(4-((2-(1,1-difluoroethyl)-6-methylpyrimidin-4-yl)amino)-5-(2-(methoxymethyl)thiazol-4-yl)pyridin-2-yl)acetamide;
N-(4-((2-(1,1-difluoroethyl)-6-methylpyrimidin-4-yl)amino)-5-(4-(methoxymethyl)thiazol-2-yl)pyridin-2-yl)acetamide;
N-(4-((2-(1,1-difluoroethyl)-6-methylpyrimidin-4-yl)amino)-5-(2-morpholinothiazol-4-yl)pyridin-2-yl)acetamide;
N-(4-((2-(1,1-difluoroethyl)-6-methylpyrimidin-4-yl)amino)-5-(1,2,4-thiadiazol-5-yl)pyridin-2-yl)acetamide;
N-(4-((2-(1,1-difluoroethyl)-6-methylpyrimidin-4-yl)amino)-5-(5-(dimethylamino)-1,3,4-thiadiazol-2-yl)pyridin-2-yl)acetamide;
N-(4-((2-(1,1-difluoroethyl)-6-methylpyrimidin-4-yl)amino)-5-(5-methyl-4,5,6,7-tetrahydrothiazolo[4,5-c]pyridin-2-yl)pyridin-2-yl)acetamide;
N-(4-((2-(1,1-difluoroethyl)-6-methylpyrimidin-4-yl)amino)-5-(5-methyl-4,5,6,7-tetrahydrothiazolo[5,4-c]pyridin-2-yl)pyridin-2-yl)acetamide;
N-(4-((2-(1,1-difluoroethyl)-6-methylpyrimidin-4-yl)amino)-5-(7-methyl-5,6,7,8-tetrahydroimidazo[1,2-a]pyrazin-2-yl)pyridin-2-yl)acetamide;
N-(4-((2-(1,1-difluoroethyl)-6-methylpyrimidin-4-yl)amino)-5-(4-methyl-4,5,6,7-tetrahydropyrazolo[1,5-a]pyrimidin-2-yl)pyridin-2-yl)acetamide;
N-(5-(6-(2-cyanopropan-2-yl)pyrimidin-4-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(6-isopropoxypyrimidin-4-yl)pyridin-2-yl)acetamide;
N-(4-((2-(1,1-difluoroethyl)-6-methylpyrimidin-4-yl)amino)-5-(6-ethoxypyrimidin-4-yl)pyridin-2-yl)acetamide;
N-(4-((2-(1,1-difluoroethyl)-6-methylpyrimidin-4-yl)amino)-5-(6-ethylpyrimidin-4-yl)pyridin-2-yl)acetamide;
N-(4-((2-(1,1-difluoroethyl)-6-methylpyrimidin-4-yl)amino)-5-(6-isopropylpyrimidin-4-yl)pyridin-2-yl)acetamide;
N-(4-((2-(1,1-difluoroethyl)-6-methylpyrimidin-4-yl)amino)-5-(6-(difluoromethyl)pyrimidin-4-yl)pyridin-2-yl)acetamide;
N-(4-((2-(1,1-difluoroethyl)-6-methylpyrimidin-4-yl)amino)-5-(6-(dimethylamino)pyrimidin-4-yl)pyridin-2-yl)acetamide;
N-(4-((2-(1,1-difluoroethyl)-6-methylpyrimidin-4-yl)amino)-5-(6-(4-methylpiperazin-1-yl)pyrimidin-4-yl)pyridin-2-yl)acetamide;
N-(4-((2-(1,1-difluoroethyl)-6-methylpyrimidin-4-yl)amino)-5-(6-(2-methoxyethoxy)pyrimidin-4-yl)pyridin-2-yl)acetamide;
N-(4-((2-(1,1-difluoroethyl)-6-methylpyrimidin-4-yl)amino)-5-(1-(2-methoxyethyl)-6-oxo-1,6-dihydropyrimidin-4-yl)pyridin-2-yl)acetamide;
N-(4-((2-(1,1-difluoroethyl)-6-methylpyrimidin-4-yl)amino)-5-(pyridazin-3-yl)pyridin-2-yl)acetamide;
N-(4-((2-(1,1-difluoroethyl)-6-methylpyrimidin-4-yl)amino)-5-(5-methylpyridazin-3-yl)pyridin-2-yl)acetamide;
N-(4-((2-(1,1-difluoroethyl)-6-methylpyrimidin-4-yl)amino)-5-(5-(dimethylamino)pyridazin-3-yl)pyridin-2-yl)acetamide;
N-(4-((2-(1,1-difluoroethyl)-6-methylpyrimidin-4-yl)amino)-5-(5-(isopropylamino)pyridazin-3-yl)pyridin-2-yl)acetamide;
N-(4-((2-(1,1-difluoroethyl)pyrimidin-4-yl)amino)-5-(5-morpholino-1,3,4-thiadiazol-2-yl)pyridin-2-yl)acetamide;
N-(4-((2-(1,1-difluoroethyl)pyrimidin-4-yl)amino)-5-(6-(dimethylamino)pyrimidin-4-yl)pyridin-2-yl)acetamide;
N-(4-((2-(1,1-difluoroethyl)-6-methylpyrimidin-4-yl)amino)-5-(1-(2-methoxyethyl)-1H-pyrazol-3-yl)pyridin-2-yl)acetamide;
N-(4-((2-(1,1-difluoroethyl)-6-methylpyrimidin-4-yl)amino)-5-(1-(2-methoxyethyl)-1H-pyrazol-5-yl)pyridin-2-yl)acetamide;
N-(4-((2-(1,1-difluoroethyl)-6-methylpyrimidin-4-yl)amino)-5-(1-(2-methoxyethyl)-1H-1,2,3-triazol-4-yl)pyridin-2-yl)acetamide;
N-(4-((2-(1,1-difluoroethyl)-6-methylpyrimidin-4-yl)amino)-5-(2-(2-methoxyethyl)-2H-1,2,3-triazol-4-yl)pyridin-2-yl)acetamide;
N-(4-((2-(1,1-difluoroethyl)-6-ethylpyrimidin-4-yl)amino)-5-(2-(2-methoxyethyl)-2H-1,2,3-triazol-4-yl)pyridin-2-yl)acetamide;
N-(4-((2-(1,1-difluoroethyl)-6-ethylpyrimidin-4-yl)amino)-5-(1-(2-methoxyethyl)-1H-1,2,3-triazol-4-yl)pyridin-2-yl)acetamide;
N-(4-((2-(1,1-difluoroethyl)-6-methylpyrimidin-4-yl)amino)-5-(1-(2-methoxyethyl)-1H-1,2,4-triazol-3-yl)pyridin-2-yl)acetamide;
N-(4-((2-(1,1-difluoroethyl)-6-methylpyrimidin-4-yl)amino)-5-(6-(2-methoxypropan-2-yl)pyrimidin-4-yl)pyridin-2-yl)acetamide;
N-(4′-((2-(1,1-difluoroethyl)pyrimidin-4-yl)amino)-5-((difluoromethoxy)methyl)-[2,3′-bipyridin]-6′-yl)acetamide;
N-(4′-((2-(1,1-difluoroethyl)-6-ethylpyrimidin-4-yl)amino)-5-((dimethylamino)methyl)-[2,3′-bipyridin]-6′-yl)acetamide;
N-(4′-((2-(1,1-difluoroethyl)-6-ethylpyrimidin-4-yl)amino)-5-morpholino-[2,3′-bipyridin]-6′-yl)acetamide;
N-(4′-((2-(1,1-difluoroethyl)-6-ethylpyrimidin-4-yl)amino)-5-(morpholinomethyl)-[2,3′-bipyridin]-6′-yl)acetamide;
N-(4-((2-(1,1-difluoroethyl)-6-ethylpyrimidin-4-yl)amino)-5-(6-(2-methoxyethoxy)pyridazin-3-yl)pyridin-2-yl)acetamide;
N-(4-((2-(1,1-difluoroethyl)-6-methylpyrimidin-4-yl)amino)-5-(pyrimidin-4-yl)pyridin-2-yl)acetamide;
N-(4-((2-(1,1-difluoroethyl)-6-methylpyrimidin-4-yl)amino)-5-(2-(dimethylamino)thiazol-4-yl)pyridin-2-yl)acetamide;
N-(4-((2-(1,1-difluoroethyl)-6-methylpyrimidin-4-yl)amino)-5-(5-(2-hydroxypropan-2-yl)-1-methyl-1H-pyrazol-3-yl)pyridin-2-yl)acetamide;
N-(4-((2-(1,1-difluoroethyl)pyrimidin-4-yl)amino)-5-(6-methoxypyrazin-2-yl)pyridin-2-yl)acetamide;
N-(4-((2-(1,1-difluoroethyl)pyrimidin-4-yl)amino)-5-(6-((2-methoxyethyl)amino)pyrazin-2-yl)pyridin-2-yl)acetamide;
N-(4-((2-(1,1-difluoroethyl)pyrimidin-4-yl)amino)-5-(6-((2-methoxyethyl)(methyl)amino)pyrazin-2-yl)pyridin-2-yl)acetamide;
N-(4′-((2-(1,1-difluoroethyl)pyrimidin-4-yl)amino)-5-(dimethylamino)-[2,3′-bipyridin]-6′-yl)acetamide;
N-(4-((2-(1,1-difluoroethyl)-6-methylpyrimidin-4-yl)amino)-5-(5-methyl-4,5,6,7-tetrahydro-2H-pyrazolo[4,3-c]pyridin-2-yl)pyridin-2-yl)acetamide;
(R)-N-(4-((2-(1,1-difluoroethyl)-6-methylpyrimidin-4-yl)amino)-5-(tetrahydrofuran-2-yl)pyridin-2-yl)acetamide;
(S)—N-(4-((2-(1,1-difluoroethyl)-6-methylpyrimidin-4-yl)amino)-5-(tetrahydrofuran-2-yl)pyridin-2-yl)acetamide;
N-(4′-((2-(2-oxabicyclo[2.1.1]hexan-1-yl)-6-methylpyrimidin-4-yl)amino)-5-(methoxymethyl)-[2,3′-bipyridin]-6′-yl)acetamide;
N-(4-((2-(1,1-difluoroethyl)-6-ethylpyrimidin-4-yl)amino)-5-(3-methyl-1,2,4-oxadiazol-5-yl)pyridin-2-yl)acetamide;
N-(4-((2-(1,1-difluoroethyl)-6-methylpyrimidin-4-yl)amino)-5-(4,5,6,7-tetrahydropyrazolo[1,5-a]pyrazin-2-yl)pyridin-2-yl)acetamide;
N-(4-((2-(1,1-difluoroethyl)-6-methylpyrimidin-4-yl)amino)-5-(5-ethyl-4,5,6,7-tetrahydropyrazolo[1,5-a]pyrazin-2-yl)pyridin-2-yl)acetamide;
N-(4-((2-(1,1-difluoroethyl)pyrimidin-4-yl)amino)-5-(5-methyl-5,6-dihydro-4H-pyrrolo[3,4-d]thiazol-2-yl)pyridin-2-yl)acetamide;
N-(5-(5-(cyanomethyl)-4,5,6,7-tetrahydrothiazolo[5,4-c]pyridin-2-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(5-(pyrrolidin-1-ylmethyl)pyrimidin-2-yl)pyridin-2-yl)acetamide;
N-(5-(5-cyanopyridazin-3-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(5-((dimethylamino)methyl)-1-methyl-1H-pyrazol-3-yl)pyridin-2-yl)acetamide;
1-(4-((2-(1,1-difluoroethyl)-6-methylpyrimidin-4-yl)amino)-5-(1-methyl-1H-pyrazol-3-yl)pyridin-2-yl)urea;
1-(4-((2-(1,1-difluoroethyl)-6-methylpyrimidin-4-yl)amino)-5-(1-methyl-1H-pyrazol-3-yl)pyridin-2-yl)-3-methylurea;
1-(4′-((2-(1,1-difluoroethyl)-6-methylpyrimidin-4-yl)amino)-5-fluoro-[2,3′-bipyridin]-6′-yl)-3-methylurea;
1-(4′-((2-(1,1-difluoroethyl)-6-methylpyrimidin-4-yl)amino)-5-fluoro-[2,3′-bipyridin]-6′-yl)urea;
1-(5-cyano-4′-((2-(1,1-difluoroethyl)-6-methylpyrimidin-4-yl)amino)-[2,3′-bipyridin]-6′-yl)urea;
(S)—N-(4-((2-(1,1-difluoroethyl)-6-methylpyrimidin-4-yl)amino)-5-((tetrahydrofuran-2-yl)methoxy)pyridin-2-yl)acetamide;
(R)-N-(4-((2-(1,1-difluoroethyl)-6-methylpyrimidin-4-yl)amino)-5-((tetrahydrofuran-2-yl)methoxy)pyridin-2-yl)acetamide;
N-(4-((2-(1,1-difluoroethyl)-6-methylpyrimidin-4-yl)amino)-5-(3-fluoropropoxy)pyridin-2-yl)acetamide;
N-(4-((2-(1,1-difluoroethyl)-6-(5-methoxypyridin-3-yl)pyrimidin-4-yl)amino)-5-methoxypyridin-2-yl)acetamide;
N-(4-((6-((2-oxaspiro[3.3]heptan-6-yl)oxy)-2-(1,1-difluoroethyl)pyrimidin-4-yl)amino)-5-ethoxypyridin-2-yl)acetamide;
N-(4-((6-cyclopropoxy-2-(1,1-difluoroethyl)pyrimidin-4-yl)amino)-5-(2-methoxyethoxy)pyridin-2-yl)acetamide;
(S)—N-(4-((6-cyclopropoxy-2-(1,1-difluoroethyl)pyrimidin-4-yl)amino)-5-(2-methoxypropoxy)pyridin-2-yl)acetamide;
N-(4-((2-(1,1-difluoroethyl)-6-isopropylpyrimidin-4-yl)amino)-5-(2-methoxyethoxy)pyridin-2-yl)acetamide;
N-(4-((6-cyclopropoxy-2-(1,1-difluoroethyl)pyrimidin-4-yl)amino)-5-((1-methyl-1H-pyrazol-4-yl)oxy)pyridin-2-yl)acetamide;
N-(4-((2-(1,1-difluoroethyl)-6-(1-methyl-1H-pyrazol-4-yl)pyrimidin-4-yl)amino)-5-ethoxypyridin-2-yl)acetamide;
N-(4-((2-(1,1-difluoroethyl)-6-(1-(difluoromethyl)-1H-pyrazol-4-yl)pyrimidin-4-yl)amino)-5-ethoxypyridin-2-yl)acetamide;
N-(4-((6-(1-cyclopropyl-1H-pyrazol-4-yl)-2-(1,1-difluoroethyl)pyrimidin-4-yl)amino)-5-ethoxypyridin-2-yl)acetamide;
2-(4-(6-((2-acetamido-5-ethoxypyridin-4-yl)amino)-2-(1,1-difluoroethyl)pyrimidin-4-yl)-1H-pyrazol-1-yl)acetic acid;
N-(4-((2-(1,1-difluoroethyl)-6-(1-isopropyl-1H-pyrazol-4-yl)pyrimidin-4-yl)amino)-5-ethoxypyridin-2-yl)acetamide;
tert-butyl 4-(6-((2-acetamido-5-ethoxypyridin-4-yl)amino)-2-(1,1-difluoroethyl)pyrimidin-4-yl)-1H-pyrazole-1-carboxylate;
N-(4-((2-(1,1-difluoroethyl)-6-(1-(2-(dimethylamino)ethyl)-1H-pyrazol-4-yl)pyrimidin-4-yl)amino)-5-ethoxypyridin-2-yl)acetamide;
N-(4-((2-(1,1-difluoroethyl)-6-(1-(2,2,2-trifluoroethyl)-1H-pyrazol-4-yl)pyrimidin-4-yl)amino)-5-ethoxypyridin-2-yl)acetamide;
N-(4-((2-(1,1-difluoroethyl)-6-(1-(2-methoxyethyl)-1H-pyrazol-4-yl)pyrimidin-4-yl)amino)-5-ethoxypyridin-2-yl)acetamide;
N-(4-((2-(1,1-difluoroethyl)-6-(1-ethyl-1H-pyrazol-4-yl)pyrimidin-4-yl)amino)-5-ethoxypyridin-2-yl)acetamide;
N-(4-((6-(1-cyclobutyl-1H-pyrazol-4-yl)-2-(1,1-difluoroethyl)pyrimidin-4-yl)amino)-5-ethoxypyridin-2-yl)acetamide;
N-(4-((2-(1,1-difluoroethyl)-6-(1-(2-morpholinoethyl)-1H-pyrazol-4-yl)pyrimidin-4-yl)amino)-5-ethoxypyridin-2-yl)acetamide;
N-(4-((2-(1,1-difluoroethyl)-6-(1-(methyl-d3)-1H-pyrazol-4-yl)pyrimidin-4-yl)amino)-5-ethoxypyridin-2-yl)acetamide;
N-(4-((2-(1,1-difluoroethyl)-6-isopropylpyrimidin-4-yl)amino)-5-(methoxy-d3)pyridin-2-yl)acetamide;
N-(5-(2-cyclopropoxyethoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((4-methyltetrahydro-2H-pyran-4-yl)methoxy)pyridin-2-yl)acetamide;
(S)—N-(5-(2-(tert-butoxy)propoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((3-ethylisoxazol-5-yl)methoxy)pyridin-2-yl)acetamide;
N-(4-((2-(1,1-difluoroethyl)-6-methylpyrimidin-4-yl)amino)-5-((2-methyloxazol-5-yl)methoxy)pyridin-2-yl)acetamide;
(R)-N-(5-(2-(tert-butoxy)propoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((3-methoxyisoxazol-5-yl)methoxy)pyridin-2-yl)acetamide;
N-(4-((2-(1,1-difluoroethyl)-6-methylpyrimidin-4-yl)amino)-5-(isothiazol-3-ylmethoxy)pyridin-2-yl)acetamide;
N-(5-((3-oxabicyclo[3.1.1]heptan-1-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((4-methyloxazol-2-yl)methoxy)pyridin-2-yl)acetamide;
N-(4-((2-(1,1-difluoroethyl)-6-methylpyrimidin-4-yl)amino)-5-((1-(methoxymethyl)cyclopropyl)methoxy)pyridin-2-yl)acetamide;
N-(4-((2-(1,1-difluoroethyl)-6-methylpyrimidin-4-yl)amino)-5-((5-ethyloxazol-2-yl)methoxy)pyridin-2-yl)acetamide;
N-(4-((2-(1,1-difluoroethyl)-6-methylpyrimidin-4-yl)amino)-5-(2-(2-fluoropyridin-3-yl)ethoxy)pyridin-2-yl)acetamide;
N-(4-((2-(1,1-difluoroethyl)-6-methylpyrimidin-4-yl)amino)-5-((5-methylthiazol-4-yl)methoxy)pyridin-2-yl)acetamide;
N-(4-((2-(1,1-difluoroethyl)-6-methylpyrimidin-4-yl)amino)-5-((4,6-dimethylpyrimidin-5-yl)methoxy)pyridin-2-yl)acetamide;
N-(4-((2-(1,1-difluoroethyl)-6-methylpyrimidin-4-yl)amino)-5-((3-ethyl-1,2,4-oxadiazol-5-yl)methoxy)pyridin-2-yl)acetamide;
N-(4-((2-(1,1-difluoroethyl)-6-methylpyrimidin-4-yl)amino)-5-(((1r,3r)-3-fluorocyclobutyl)methoxy)pyridin-2-yl)acetamide;
N-(5-((5-cyanofuran-2-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((4-methylthiazol-5-yl)methoxy)pyridin-2-yl)acetamide;
N-(4-((2-(1,1-difluoroethyl)-6-methylpyrimidin-4-yl)amino)-5-((3-methylisoxazol-4-yl)methoxy)pyridin-2-yl)acetamide;
(S)—N-(4-((2-(1,1-difluoroethyl)-6-methylpyrimidin-4-yl)amino)-5-(3-fluorobutoxy)pyridin-2-yl)acetamide;
N-(4-((2-(1,1-difluoroethyl)-6-methylpyrimidin-4-yl)amino)-5-((4-methoxytetrahydro-2H-pyran-4-yl)methoxy)pyridin-2-yl)acetamide;
N-(4-((2-(1,1-difluoroethyl)-6-methylpyrimidin-4-yl)amino)-5-((4,5-dimethyloxazol-2-yl)methoxy)pyridin-2-yl)acetamide;
(R)-N-(4-((2-(1,1-difluoroethyl)-6-methylpyrimidin-4-yl)amino)-5-(3-fluorobutoxy)pyridin-2-yl)acetamide;
N-(4-((2-(1,1-difluoroethyl)-6-methylpyrimidin-4-yl)amino)-5-((4-(fluoromethyl)tetrahydro-2H-pyran-4-yl)methoxy)pyridin-2-yl)acetamide;
N-(4-((2-(1,1-difluoroethyl)-6-methylpyrimidin-4-yl)amino)-5-((5-methyloxazol-2-yl)methoxy)pyridin-2-yl)acetamide;
N-(5-((5-cyclopropyloxazol-2-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((3-methyl-1,2,4-thiadiazol-5-yl)methoxy)pyridin-2-yl)acetamide;
N-(5-((2-cyclopropyloxazol-5-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((4-methoxypyrimidin-2-yl)methoxy)pyridin-2-yl)acetamide;
N-(4-((2-(1,1-difluoroethyl)-6-methylpyrimidin-4-yl)amino)-5-((6-methoxypyrimidin-4-yl)methoxy)pyridin-2-yl)acetamide;
N-(4-((2-(1,1-difluoroethyl)-6-methylpyrimidin-4-yl)amino)-5-(((1s,4s)-4-methyl-2-oxabicyclo[2.2.2]octan-1-yl)methoxy)pyridin-2-yl)acetamide;
N-(4-((6-cyclopropoxy-2-(1,1-difluoroethyl)pyrimidin-4-yl)amino)-5-(pyridin-2-ylmethoxy)pyridin-2-yl)acetamide;
N-(4-((6-cyclopropoxy-2-(1,1-difluoroethyl)pyrimidin-4-yl)amino)-5-((3,4-difluorobenzyl)oxy)pyridin-2-yl)acetamide;
N-(4-((6-cyclopropoxy-2-(1,1-difluoroethyl)pyrimidin-4-yl)amino)-5-((1r,3r)-3-methoxycyclobutoxy)pyridin-2-yl)acetamide;
N-(4-((6-cyclopropoxy-2-(1,1-difluoroethyl)pyrimidin-4-yl)amino)-5-((1s,3s)-3-methoxycyclobutoxy)pyridin-2-yl)acetamide;
N-(4-((6-cyclopropoxy-2-(1,1-difluoroethyl)pyrimidin-4-yl)amino)-5-((1r,3r)-3-fluorocyclobutoxy)pyridin-2-yl)acetamide;
N-(4-((6-cyclopropoxy-2-(1,1-difluoroethyl)pyrimidin-4-yl)amino)-5-((1s,3s)-3-fluorocyclobutoxy)pyridin-2-yl)acetamide;
N-(5-((1-cyclobutyl-1H-pyrazol-4-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(pyridin-3-ylmethoxy)pyridin-2-yl)acetamide;
N-(4-((2-(1,1-difluoroethyl)-6-methylpyrimidin-4-yl)amino)-5-((5-methoxypyrazin-2-yl)oxy)pyridin-2-yl)acetamide;
N-(4-((2-(1,1-difluoroethyl)-6-methylpyrimidin-4-yl)amino)-5-((6-methoxypyrazin-2-yl)oxy)pyridin-2-yl)acetamide;
N-(4-((2-(1,1-difluoroethyl)-6-methylpyrimidin-4-yl)amino)-5-((6-methoxypyridazin-3-yl)oxy)pyridin-2-yl)acetamide;
N-(4-((2-(1,1-difluoroethyl)-6-methylpyrimidin-4-yl)amino)-5-((2-(trifluoromethyl)pyrimidin-4-yl)oxy)pyridin-2-yl)acetamide;
methyl (4-((2-(1,1-difluoroethyl)-6-methylpyrimidin-4-yl)amino)-5-ethoxypyridin-2-yl)carbamate;
N-(4-((2-(1,1-difluoroethyl)-6-(1-isopropyl-1H-pyrazol-4-yl)pyrimidin-4-yl)amino)-5-(ethoxy-d5)pyridin-2-yl)acetamide;
N-(4-((2-(1,1-difluoroethyl)-6-isopropylpyrimidin-4-yl)amino)-5-(ethoxy-d5)pyridin-2-yl)acetamide;
N-(4-((2-(1,1-difluoroethyl)-6-(1-isopropyl-1H-pyrazol-4-yl)pyrimidin-4-yl)amino)-5-methoxypyridin-2-yl)acetamide;
N-(4-((2-(1,1-difluoroethyl)-6-isopropylpyrimidin-4-yl)amino)-5-(ethoxy-1,1-d2)pyridin-2-yl)acetamide;
N-(4-((2-(1,1-difluoroethyl)-6-isopropylpyrimidin-4-yl)amino)-5-(pyridin-2-ylmethoxy)pyridin-2-yl)acetamide;
N-(4-((2-(1,1-difluoroethyl)-6-(1-isopropyl-1H-pyrazol-4-yl)pyrimidin-4-yl)amino)-5-(2-methoxyethoxy)pyridin-2-yl)acetamide;
N-(4-((2-(1,1-difluoroethyl)-6-(5-methylpyridin-3-yl)pyrimidin-4-yl)amino)-5-ethoxypyridin-2-yl)acetamide;
N-(4-((2-(1,1-difluoroethyl)-6-(6-ethylpyridin-3-yl)pyrimidin-4-yl)amino)-5-ethoxypyridin-2-yl)acetamide;
N-(4-((2-(1,1-difluoroethyl)-6-(6-methoxypyridin-3-yl)pyrimidin-4-yl)amino)-5-ethoxypyridin-2-yl)acetamide;
N-(4-((2-(1,1-difluoroethyl)-6-(5-methoxypyridin-3-yl)pyrimidin-4-yl)amino)-5-ethoxypyridin-2-yl)acetamide;
N-(4-((2-(1,1-difluoroethyl)-6-(1-isobutyl-1H-pyrazol-4-yl)pyrimidin-4-yl)amino)-5-ethoxypyridin-2-yl)acetamide;
N-(4-((6-chloro-2-(1,1-difluoroethyl)pyrimidin-4-yl)amino)-5-ethoxypyridin-2-yl)acetamide;
N-(4-((2-(1,1-difluoroethyl)-6-(3-fluoropyridin-4-yl)pyrimidin-4-yl)amino)-5-methoxypyridin-2-yl)acetamide;
N-(4-((6-(1-cyclobutyl-1H-pyrazol-4-yl)-2-(1,1-difluoroethyl)pyrimidin-4-yl)amino)-5-methoxypyridin-2-yl)acetamide;
N-(4-((2-(1,1-difluoroethyl)-6-(1-(difluoromethyl)-1H-pyrazol-4-yl)pyrimidin-4-yl)amino)-5-methoxypyridin-2-yl)acetamide;
N-(4-((6-(1-cyclopropyl-1H-pyrazol-4-yl)-2-(1,1-difluoroethyl)pyrimidin-4-yl)amino)-5-methoxypyridin-2-yl)acetamide;
N-(4-((2-(1,1-difluoroethyl)-6-(1-(oxetan-3-yl)-1H-pyrazol-4-yl)pyrimidin-4-yl)amino)-5-methoxypyridin-2-yl)acetamide;
N-(4-((6-(1-(cyclopropylmethyl)-1H-pyrazol-4-yl)-2-(1,1-difluoroethyl)pyrimidin-4-yl)amino)-5-methoxypyridin-2-yl)acetamide;
N-(4-((2-(1,1-difluoroethyl)-6-(1-(2-morpholinoethyl)-1H-pyrazol-4-yl)pyrimidin-4-yl)amino)-5-methoxypyridin-2-yl)acetamide;
N-(4-((2-(1,1-difluoroethyl)-6-(1-(2-(dimethylamino)ethyl)-1H-pyrazol-4-yl)pyrimidin-4-yl)amino)-5-methoxypyridin-2-yl)acetamide;
N-(4-((2-(1,1-difluoroethyl)-6-(1-(2,2,2-trifluoroethyl)-1H-pyrazol-4-yl)pyrimidin-4-yl)amino)-5-methoxypyridin-2-yl)acetamide;
N-(4-((2-(1,1-difluoroethyl)-6-(pyridin-4-yl)pyrimidin-4-yl)amino)-5-methoxypyridin-2-yl)acetamide;
N-(4-((2-(1,1-difluoroethyl)-6-(5-fluoro-1-isopropyl-1H-pyrazol-4-yl)pyrimidin-4-yl)amino)-5-methoxypyridin-2-yl)acetamide;
N-(4-((2-(1,1-difluoroethyl)-6-(5-fluoropyridin-2-yl)pyrimidin-4-yl)amino)-5-methoxypyridin-2-yl)acetamide;
N-(4-((2-(1,1-difluoroethyl)-6-(5-fluoro-1H-pyrazol-4-yl)pyrimidin-4-yl)amino)-5-methoxypyridin-2-yl)acetamide;
N-(4-((2-(1,1-difluoroethyl)-6-(1-(2-methoxyethyl)-1H-pyrazol-4-yl)pyrimidin-4-yl)amino)-5-methoxypyridin-2-yl)acetamide;
N-(4-((2-(1,1-difluoroethyl)-6-(2-methylthiazol-5-yl)pyrimidin-4-yl)amino)-5-methoxypyridin-2-yl)acetamide;
N-(4-((2-(1,1-difluoroethyl)-6-(pyridin-3-yl)pyrimidin-4-yl)amino)-5-methoxypyridin-2-yl)acetamide;
N-(4-((2-(1,1-difluoroethyl)-6-(5-methylpyridin-3-yl)pyrimidin-4-yl)amino)-5-methoxypyridin-2-yl)acetamide;
N-(4-((2-(1,1-difluoroethyl)-2′-morpholino-[4,5′-bipyrimidin]-6-yl)amino)-5-methoxypyridin-2-yl)acetamide;
N-(4-((2-(1,1-difluoroethyl)-6-(6-methoxypyridin-3-yl)pyrimidin-4-yl)amino)-5-methoxypyridin-2-yl)acetamide;
N-(4-((2-(1,1-difluoroethyl)-6-(1-isobutyl-1H-pyrazol-4-yl)pyrimidin-4-yl)amino)-5-methoxypyridin-2-yl)acetamide;
N-(4-((6-(4-cyanophenyl)-2-(1,1-difluoroethyl)pyrimidin-4-yl)amino)-5-methoxypyridin-2-yl)acetamide;
N-(4-((6-(3-cyano-4-fluorophenyl)-2-(1,1-difluoroethyl)pyrimidin-4-yl)amino)-5-methoxypyridin-2-yl)acetamide;
N-(4-((2-(1,1-difluoroethyl)-6-(1-methyl-3-(trifluoromethyl)-1H-pyrazol-4-yl)pyrimidin-4-yl)amino)-5-methoxypyridin-2-yl)acetamide;
N-(4-((2-(1,1-difluoroethyl)-6-(7-fluoro-2-methyl-2H-indazol-5-yl)pyrimidin-4-yl)amino)-5-methoxypyridin-2-yl)acetamide;
N-(4-((2-(1,1-difluoroethyl)-6-(2,8-dimethyl-[1,2,4]triazolo[1,5-a]pyridin-6-yl)pyrimidin-4-yl)amino)-5-methoxypyridin-2-yl)acetamide;
N-(4-((2-(1,1-difluoroethyl)-6-(8-fluoro-2-methylimidazo[1,2-a]pyridin-6-yl)pyrimidin-4-yl)amino)-5-methoxypyridin-2-yl)acetamide;
N-(4-((2-(1,1-difluoroethyl)-6-(2-methylimidazo[1,2-b]pyridazin-6-yl)pyrimidin-4-yl)amino)-5-methoxypyridin-2-yl)acetamide;
N-(4-((2-(1,1-difluoroethyl)-6-(2,7-dimethyl-2H-indazol-5-yl)pyrimidin-4-yl)amino)-5-methoxypyridin-2-yl)acetamide;
N-(4-((2-(1,1-difluoroethyl)-6-(8-methoxy-2-methylimidazo[1,2-a]pyridin-6-yl)pyrimidin-4-yl)amino)-5-methoxypyridin-2-yl)acetamide;
N-(4-((6-(8-cyano-2-methylimidazo[1,2-a]pyridin-6-yl)-2-(1,1-difluoroethyl)pyrimidin-4-yl)amino)-5-methoxypyridin-2-yl)acetamide;
N-(4-((2-(1,1-difluoroethyl)-6-(2,8-dimethylimidazo[1,2-a]pyridin-6-yl)pyrimidin-4-yl)amino)-5-methoxypyridin-2-yl)acetamide;
N-(4-((2-(1,1-difluoroethyl)-6-methylpyrimidin-4-yl)amino)-5-(2-fluoroethoxy)pyridin-2-yl)acetamide;
N-(4-((2-(1,1-difluoroethyl)-6-ethylpyrimidin-4-yl)amino)-5-(3-fluoropropoxy)pyridin-2-yl)acetamide;
N-(4-((2-(2-fluoropropan-2-yl)-6-methylpyrimidin-4-yl)amino)-5-(2-methoxyethoxy)pyridin-2-yl)acetamide;
N-(4-((2-(1,1-difluoroethyl)-6-ethylpyrimidin-4-yl)amino)-5-(2-fluoroethoxy)pyridin-2-yl)acetamide;
N-(4-((2-(1,1-difluoroethyl)-6-isopropylpyrimidin-4-yl)amino)-5-((1r,3r)-3-methoxycyclobutoxy)pyridin-2-yl)acetamide;
N-(4-((2-(1,1-difluoroethyl)-6-isopropylpyrimidin-4-yl)amino)-5-((1s,3s)-3-methoxycyclobutoxy)pyridin-2-yl)acetamide;
N-(4-((2-(1,1-difluoroethyl)-6-isopropylpyrimidin-4-yl)amino)-5-((1r,3r)-3-fluorocyclobutoxy)pyridin-2-yl)acetamide;
N-(4-((2-(1,1-difluoroethyl)-6-isopropylpyrimidin-4-yl)amino)-5-((1s,3s)-3-fluorocyclobutoxy)pyridin-2-yl)acetamide;
N-(4-((2-(1,1-difluoroethyl)-6-methylpyrimidin-4-yl)amino)-5-((1s,3s)-3-fluorocyclobutoxy)pyridin-2-yl)acetamide;
N-(4-((2-(1,1-difluoroethyl)-6-ethylpyrimidin-4-yl)amino)-5-((1s,3s)-3-fluorocyclobutoxy)pyridin-2-yl)acetamide;
N-(4-((2-(1,1-difluoroethyl)-6-(1-isopropyl-1H-pyrazol-4-yl)pyrimidin-4-yl)amino)-5-((1s,3s)-3-fluorocyclobutoxy)pyridin-2-yl)acetamide;
N-(4-((6-cyclopropoxy-2-(1,1-difluoroethyl)pyrimidin-4-yl)amino)-5-(methoxy-d3)pyridin-2-yl)acetamide;
N-(4-((2-(1,1-difluoroethyl)-6-(1H-pyrazol-4-yl)pyrimidin-4-yl)amino)-5-methoxypyridin-2-yl)acetamide;
(R)-N-(4-((2-(1,1-difluoroethyl)-6-methylpyrimidin-4-yl)amino)-5-((3-fluorotetrahydro-2H-pyran-3-yl)methoxy)pyridin-2-yl)acetamide;
N-(4-((2-(1,1-difluoroethyl)-6-methylpyrimidin-4-yl)amino)-5-(((2S,6S)-6-methyltetrahydro-2H-pyran-2-yl)methoxy)pyridin-2-yl)acetamide;
(S)—N-(4-((2-(1,1-difluoroethyl)-6-methylpyrimidin-4-yl)amino)-5-((6,6-dimethyl-1,4-dioxan-2-yl)methoxy)pyridin-2-yl)acetamide;
(S)—N-(5-(2-cyclobutyl-2-fluoroethoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(((1r,4s)-1-(fluoromethyl)-2-oxabicyclo[2.1.1]hexan-4-yl)methoxy)pyridin-2-yl)acetamide;
(R)-N-(4-((2-(1,1-difluoroethyl)-6-methylpyrimidin-4-yl)amino)-5-(spiro[2.2]pentan-1-ylmethoxy)pyridin-2-yl)acetamide;
N-(5-(((1r,4r)-7-oxabicyclo[2.2.1]heptan-1-yl)methoxy)-4-((2-(1,1-difluoroethyl)-6-methylpyrimidin-4-yl)amino)pyridin-2-yl)acetamide;
(R)-N-(4-((2-(1,1-difluoroethyl)-6-methylpyrimidin-4-yl)amino)-5-(2-methoxybutoxy)pyridin-2-yl)acetamide;
N-(4-((2-(1,1-difluoroethyl)-6-methylpyrimidin-4-yl)amino)-5-(((1S,2S)-2-fluorocyclopropyl)methoxy)pyridin-2-yl)acetamide;
N-(4-((2-(1,1-difluoroethyl)-6-methylpyrimidin-4-yl)amino)-5-(((1S,2R)-2-fluorocyclopropyl)methoxy)pyridin-2-yl)acetamide;
(S)—N-(5-(2-(tert-butoxy)propoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(((1R,2S)-2-(fluoromethyl)cyclopropyl)methoxy)pyridin-2-yl)acetamide;
(R)-N-(4-((2-(1,1-difluoroethyl)-6-methylpyrimidin-4-yl)amino)-5-(2-fluoro-3-methylbutoxy)pyridin-2-yl)acetamide;
(S)—N-(4-((2-(1,1-difluoroethyl)-6-methylpyrimidin-4-yl)amino)-5-((5,5-dimethyl-1,4-dioxan-2-yl)methoxy)pyridin-2-yl)acetamide;
(S)—N-(4-((2-(1,1-difluoroethyl)-6-methylpyrimidin-4-yl)amino)-5-((3-methyltetrahydrofuran-3-yl)methoxy)pyridin-2-yl)acetamide;
N-(5-(((1R,3R)-5-oxaspiro[2.4]heptan-1-yl)methoxy)-4-((2-(1,1-difluoroethyl)-6-methylpyrimidin-4-yl)amino)pyridin-2-yl)acetamide;
(R)-N-(5-((6-oxaspiro[3.4]octan-7-yl)methoxy)-4-((2-(1,1-difluoroethyl)-6-methylpyrimidin-4-yl)amino)pyridin-2-yl)acetamide;
(R)-N-(4-((2-(1,1-difluoroethyl)-6-methylpyrimidin-4-yl)amino)-5-(3-(tetrahydrofuran-2-yl)propoxy)pyridin-2-yl)acetamide;
(R)-N-(4-((2-(1,1-difluoroethyl)-6-methylpyrimidin-4-yl)amino)-5-((2-methyltetrahydro-2H-pyran-2-yl)methoxy)pyridin-2-yl)acetamide;
N-(5-methoxy-4-((4-methyl-6-(trifluoromethyl)pyrimidin-2-yl)amino)pyridin-2-yl)acetamide;
N-(4-((6-cyclopropoxy-2-(trifluoromethyl)pyrimidin-4-yl)amino)-5-methoxypyridin-2-yl)acetamide;
N-(5-methoxy-4-((4-methoxy-6-(trifluoromethyl)pyrimidin-2-yl)amino)pyridin-2-yl)acetamide;
N-(4-((6-(2-fluoropropan-2-yl)pyrazin-2-yl)amino)-5-(2-methoxyethoxy)pyridin-2-yl)acetamide;
(R)-N-(4-((2-(1,1-difluoroethyl)-6-methylpyrimidin-4-yl)amino)-5-(3-methoxy-2-methylpropoxy)pyridin-2-yl)acetamide;
N-(4-((4-(2-fluoropropan-2-yl)-6-methylpyrimidin-2-yl)amino)-5-(2-methoxyethoxy)pyridin-2-yl)acetamide;
N-(4-((2-(1,1-difluoroethyl)-6-methylpyrimidin-4-yl)amino)-5-(((2R,6R)-6-methyltetrahydro-2H-pyran-2-yl)methoxy)pyridin-2-yl)acetamide;
(S)—N-(5-((5,8-dioxaspiro[3.5]nonan-6-yl)methoxy)-4-((2-(1,1-difluoroethyl)-6-methylpyrimidin-4-yl)amino)pyridin-2-yl)acetamide;
(R)-N-(4-((2-(1,1-difluoroethyl)-6-methylpyrimidin-4-yl)amino)-5-(3-fluorobutoxy)pyridin-2-yl)acetamide;
(S)—N-(5-((1,4-dioxan-2-yl)methoxy)-4-((6-cyclopropoxy-2-(1,1-difluoroethyl)pyrimidin-4-yl)amino)pyridin-2-yl)acetamide;
N-(4-((6-cyclopropoxy-2-(2-fluoropropan-2-yl)pyrimidin-4-yl)amino)-5-(2-methoxyethoxy)pyridin-2-yl)acetamide;
methyl (4-((6-cyclopropoxy-2-(1,1-difluoroethyl)pyrimidin-4-yl)amino)-5-(2-(dimethylamino)ethoxy)pyridin-2-yl)carbamate;
N-(4-((2-(2-oxabicyclo[2.1.1]hexan-4-yl)-6-methylpyrimidin-4-yl)amino)-5-methoxypyridin-2-yl)acetamide;
N-(5-methoxy-4-((6-methyl-2-(1-methyl-2-oxabicyclo[2.1.1]hexan-4-yl)pyrimidin-4-yl)amino)pyridin-2-yl)acetamide;
N-(5-methoxy-4-((2-(tetrahydrofuran-3-yl)pyrimidin-4-yl)amino)pyridin-2-yl)acetamide;
N-(5-methoxy-4-((6-methyl-2-(tetrahydrofuran-3-yl)pyrimidin-4-yl)amino)pyridin-2-yl)acetamide;
N-(5-methoxy-4-((2-methoxy-6-methylpyrimidin-4-yl)amino)pyridin-2-yl)acetamide;
(S)—N-(5-((1,4-dioxan-2-yl)methoxy)-4-((6-cyclopropoxy-2-(1,1-difluoroethyl)pyrimidin-4-yl)amino)pyridin-2-yl)acetamide;
(R)-N-(5-((1,4-dioxan-2-yl)methoxy)-4-((6-cyclopropoxy-2-(1,1-difluoroethyl)pyrimidin-4-yl)amino)pyridin-2-yl)acetamide;
N-(4-((6-cyclobutoxy-2-(1,1-difluoroethyl)pyrimidin-4-yl)amino)-5-methoxypyridin-2-yl)acetamide;
N-(4-((6-((1s,3s)-3-cyanocyclobutoxy)-2-(1,1-difluoroethyl)pyrimidin-4-yl)amino)-5-methoxypyridin-2-yl)acetamide;
N-(4-((2-(1,1-difluoroethyl)-6-((1r,3r)-3-fluorocyclobutoxy)pyrimidin-4-yl)amino)-5-ethoxypyridin-2-yl)acetamide;
N-(4-((2-(1,1-difluoroethyl)-6-((1s,3s)-3-fluorocyclobutoxy)pyrimidin-4-yl)amino)-5-ethoxypyridin-2-yl)acetamide;
N-(4-((6-(3,3-difluorocyclobutoxy)-2-(1,1-difluoroethyl)pyrimidin-4-yl)amino)-5-ethoxypyridin-2-yl)acetamide;
N-(4-((6-(3,3-difluorocyclobutoxy)-2-(1,1-difluoroethyl)pyrimidin-4-yl)amino)-5-methoxypyridin-2-yl)acetamide;
N-(4-((2-(1,1-difluoroethyl)-6-((1s,3s)-3-fluorocyclobutoxy)pyrimidin-4-yl)amino)-5-methoxypyridin-2-yl)acetamide;
N-(4-((6-cyclobutoxy-2-(1,1-difluoroethyl)pyrimidin-4-yl)amino)-5-ethoxypyridin-2-yl)acetamide;
N-(4-((2-(1,1-difluoroethyl)-6-((1s,3s)-3-methylcyclobutoxy)pyrimidin-4-yl)amino)-5-ethoxypyridin-2-yl)acetamide;
N-(4-((6-((1s,3s)-3-cyanocyclobutoxy)-2-(1,1-difluoroethyl)pyrimidin-4-yl)amino)-5-ethoxypyridin-2-yl)acetamide;
N-(5-((3,3-difluorocyclobutyl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((3-fluorobenzyl)oxy)pyridin-2-yl)acetamide;
N-(5-((3,4-difluorobenzyl)oxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-((2-methoxybenzyl)oxy)pyridin-2-yl)acetamide;
N-(4-((2-(1,1-difluoroethyl)-6-methylpyrimidin-4-yl)amino)-5-((2-methylpyridin-3-yl)methoxy)pyridin-2-yl)acetamide;
N-(4-((2-(1,1-difluoroethyl)-6-methylpyrimidin-4-yl)amino)-5-((4-methylpyridin-3-yl)methoxy)pyridin-2-yl)acetamide;
N-(4-((2-(1,1-difluoroethyl)-6-methylpyrimidin-4-yl)amino)-5-((3-methylpyridin-2-yl)methoxy)pyridin-2-yl)acetamide;
N-(4-((2-(1,1-difluoroethyl)-6-methylpyrimidin-4-yl)amino)-5-((3-methoxypyridin-2-yl)methoxy)pyridin-2-yl)acetamide;
N-(4-((2-(1,1-difluoroethyl)-6-methylpyrimidin-4-yl)amino)-5-(pyridin-4-ylmethoxy)pyridin-2-yl)acetamide;
N-(4-((2-(1,1-difluoroethyl)-6-methylpyrimidin-4-yl)amino)-5-((5-fluoropyridin-3-yl)methoxy)pyridin-2-yl)acetamide;
N-(4-((2-(1,1-difluoroethyl)-6-methylpyrimidin-4-yl)amino)-5-((1-methyl-1H-pyrazol-3-yl)methoxy)pyridin-2-yl)acetamide;
N-(4-((2-(1,1-difluoroethyl)-6-methylpyrimidin-4-yl)amino)-5-(2-(pyridin-2-yl)ethoxy)pyridin-2-yl)acetamide;
(R)-N-(4-((2-(1,1-difluoroethyl)-6-methylpyrimidin-4-yl)amino)-5-((tetrahydrofuran-3-yl)oxy)pyridin-2-yl)acetamide;
(S)—N-(4-((2-(1,1-difluoroethyl)-6-methylpyrimidin-4-yl)amino)-5-((1-methylpyrrolidin-3-yl)oxy)pyridin-2-yl)acetamide;
(R)-N-(4-((2-(1,1-difluoroethyl)-6-methylpyrimidin-4-yl)amino)-5-((1-methylpyrrolidin-2-yl)methoxy)pyridin-2-yl)acetamide;
(R)-N-(4-((2-(1,1-difluoroethyl)-6-methylpyrimidin-4-yl)amino)-5-((1-methylpyrrolidin-3-yl)methoxy)pyridin-2-yl)acetamide;
(S)—N-(4-((2-(1,1-difluoroethyl)-6-methylpyrimidin-4-yl)amino)-5-((tetrahydrofuran-2-yl)methoxy)pyridin-2-yl)acetamide;
N-(4-((2-(1,1-difluoroethyl)-6-methylpyrimidin-4-yl)amino)-5-(2-isopropoxyethoxy)pyridin-2-yl)acetamide;
N-(4-((2-(1,1-difluoroethyl)-6-methylpyrimidin-4-yl)amino)-5-((3-fluorooxetan-3-yl)methoxy)pyridin-2-yl)acetamide;
N-(5-(benzo[d][1,3]dioxol-5-ylmethoxy)-4-((2-(1,1-difluoroethyl)-6-methylpyrimidin-4-yl)amino)pyridin-2-yl)acetamide;
N-(4-((2-(1,1-difluoroethyl)-6-(methylamino)pyrimidin-4-yl)amino)-5-ethoxypyridin-2-yl)acetamide;
N-(4-((2-(1,1-difluoroethyl)-6-methylpyrimidin-4-yl)amino)-5-hydroxypyridin-2-yl)acetamide;
N-(5-(benzyloxy)-4-((6-cyclopropoxy-2-(1,1-difluoroethyl)pyrimidin-4-yl)amino)pyridin-2-yl)acetamide;
N-(4-((6-(cyclobutylamino)-2-(1,1-difluoroethyl)pyrimidin-4-yl)amino)-5-(ethoxy-d5)pyridin-2-yl)acetamide;
N-(4-((6-cyclopropoxy-2-(1,1-difluoroethyl)pyrimidin-4-yl)amino)-5-(ethoxy-d5)pyridin-2-yl)acetamide;
N-(4-((6-cyclopropoxy-2-(1,1-difluoroethyl)pyrimidin-4-yl)amino)-5-ethoxypyridin-2-yl)acetamide;
N-(5-ethoxy-4-((6-methoxy-5-(1-methyl-1H-pyrazol-3-yl)pyridin-2-yl)amino)pyridin-2-yl)acetamide;
N-(4-((2-(1,1-difluoroethyl)-6-methylpyrimidin-4-yl)amino)-5-(2-methoxy-2-methylpropoxy)pyridin-2-yl)acetamide;
N-(4-((2-(1,1-difluoroethyl)-6-methylpyrimidin-4-yl)amino)-5-((1-methoxycyclobutyl)methoxy)pyridin-2-yl)acetamide;
(S)—N-(5-((1-cyclopropylpyrrolidin-3-yl)methoxy)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(isoxazol-3-ylmethoxy)pyridin-2-yl)acetamide;
N-(4-((2-(1,1-difluoroethyl)-6-methylpyrimidin-4-yl)amino)-5-((1-methyl-1H-imidazol-2-yl)methoxy)pyridin-2-yl)acetamide;
N-(4-((2-(1,1-difluoroethyl)-6-methylpyrimidin-4-yl)amino)-5-((5-methylisoxazol-3-yl)methoxy)pyridin-2-yl)acetamide;
N-(4-((2-(1,1-difluoroethyl)-6-methylpyrimidin-4-yl)amino)-5-((5-methyl-1,2,4-oxadiazol-3-yl)methoxy)pyridin-2-yl)acetamide;
(R)-N-(4-((2-(1,1-difluoroethyl)-6-methylpyrimidin-4-yl)amino)-5-((5-oxotetrahydrofuran-2-yl)methoxy)pyridin-2-yl)acetamide;
(S)—N-(4-((2-(1,1-difluoroethyl)-6-methylpyrimidin-4-yl)amino)-5-((5-oxopyrrolidin-2-yl)methoxy)pyridin-2-yl)acetamide;
(S)—N-(4-((2-(1,1-difluoroethyl)-6-methylpyrimidin-4-yl)amino)-5-(2-(1-methylpyrrolidin-2-yl)ethoxy)pyridin-2-yl)acetamide;
(R)-N-(4-((2-(1,1-difluoroethyl)-6-methylpyrimidin-4-yl)amino)-5-((5-oxopyrrolidin-2-yl)methoxy)pyridin-2-yl)acetamide;
(R)-N-(4-((2-(1,1-difluoroethyl)-6-methylpyrimidin-4-yl)amino)-5-((4-isopropylmorpholin-2-yl)methoxy)pyridin-2-yl)acetamide;
N-(4-((2-(1,1-difluoroethyl)-6-methylpyrimidin-4-yl)amino)-5-(isoxazol-4-ylmethoxy)pyridin-2-yl)acetamide;
N-(4-((2-(1,1-difluoroethyl)-6-methylpyrimidin-4-yl)amino)-5-(oxazol-4-ylmethoxy)pyridin-2-yl)acetamide;
N-(4-((2-(1,1-difluoroethyl)-6-methylpyrimidin-4-yl)amino)-5-((1-methylazetidin-3-yl)methoxy)pyridin-2-yl)acetamide;
N-(4-((2-(1,1-difluoroethyl)-6-methylpyrimidin-4-yl)amino)-5-(2-ethoxyethoxy)pyridin-2-yl)acetamide;
N-(4-((2-(1,1-difluoroethyl)-6-methylpyrimidin-4-yl)amino)-5-((1-methyl-1H-pyrazol-4-yl)methoxy)pyridin-2-yl)acetamide;
N-(4-((2-(1,1-difluoroethyl)-6-methylpyrimidin-4-yl)amino)-5-((1-ethyl-1H-pyrazol-4-yl)methoxy)pyridin-2-yl)acetamide;
(S)—N-(4-((2-(1,1-difluoroethyl)-6-methylpyrimidin-4-yl)amino)-5-((1-isopropyl-5-oxopyrrolidin-3-yl)methoxy)pyridin-2-yl)acetamide;
(S)—N-(4-((2-(1,1-difluoroethyl)-6-methylpyrimidin-4-yl)amino)-5-((1-methyl-5-oxopyrrolidin-3-yl)methoxy)pyridin-2-yl)acetamide;
N-(4-((2-(1,1-difluoroethyl)-6-methylpyrimidin-4-yl)amino)-5-((3-methoxy-1-methyl-1H-pyrazol-4-yl)methoxy)pyridin-2-yl)acetamide;
N-(4-((2-(1,1-difluoroethyl)-6-methylpyrimidin-4-yl)amino)-5-(((1R,3S)-3-methoxycyclopentyl)oxy)pyridin-2-yl)acetamide;
N-(4-((2-(1,1-difluoroethyl)-6-methylpyrimidin-4-yl)amino)-5-(((1s,4s)-4-methyl-2-oxabicyclo[2.1.1]hexan-1-yl)methoxy)pyridin-2-yl)acetamide;
N-(4-((2-(1,1-difluoroethyl)-6-ethylpyrimidin-4-yl)amino)-5-(ethoxy-d5)pyridin-2-yl)acetamide;
N-(4-((2-(1,1-difluoropropyl)-6-methylpyrimidin-4-yl)amino)-5-ethoxypyridin-2-yl)acetamide;
N-(4-((2-(1,1-difluoroethyl)-5-methoxypyrimidin-4-yl)amino)-5-ethoxypyridin-2-yl)acetamide;
(R)-N-(4-((2-(2,2-dimethylcyclopropyl)-6-methylpyrimidin-4-yl)amino)-5-ethoxypyridin-2-yl)acetamide;
N-(5-ethoxy-4-((6-((1r,3r)-3-methoxycyclobutoxy)-2-methylpyrimidin-4-yl)amino)pyridin-2-yl)acetamide;
1-(5-cyano-4′-((2-(1,1-difluoroethyl)-6-methylpyrimidin-4-yl)amino)-[2,3′-bipyridin]-6′-yl)-3-methylurea;
N-(4-((2-(1,1-difluoroethyl)-6-ethylpyrimidin-4-yl)amino)-5-(4-methyl-1,3,5-triazin-2-yl)pyridin-2-yl)acetamide;
N-(4′-((2-(1,1-difluoroethyl)-6-methylpyrimidin-4-yl)amino)-5-(2-methoxyethoxy)-[2,3′-bipyridin]-6′-yl)acetamide;
N-(4′-((2-(1,1-difluoroethyl)pyrimidin-4-yl)amino)-5-(2-methoxyethoxy)-[2,3′-bipyridin]-6′-yl)acetamide;
N-(4′-((2-(1,1-difluoroethyl)-6-methylpyrimidin-4-yl)amino)-5-((difluoromethoxy)methyl)-[2,3′-bipyridin]-6′-yl)acetamide;
N-(5-cyano-4′-((6-(1,1-difluoroethyl)pyridin-2-yl)amino)-[2,3′-bipyridin]-6′-yl)acetamide;
N-(4-((6-(1,1-difluoroethyl)pyridin-2-yl)amino)-5-(6-methoxypyridazin-3-yl)pyridin-2-yl)acetamide;
N-(4-((2-(1,1-difluoroethyl)pyrimidin-4-yl)amino)-5-(6-methoxypyridazin-3-yl)pyridin-2-yl)acetamide-2,2,2-d3;
N-(4-((2-(1,1-difluoroethyl)-6-methylpyrimidin-4-yl)amino)-5-(6,7-dihydro-4H-pyrazolo[5,1-c][1,4]oxazin-2-yl)pyridin-2-yl)acetamide;
N-(4-((2-(1,1-difluoroethyl)pyrimidin-4-yl)amino)-5-(6,7-dihydro-4H-pyrazolo[5,1-c][1,4]oxazin-2-yl)pyridin-2-yl)acetamide;
N-(5-(6,7-dihydro-4H-pyrazolo[5,1-c][1,4]oxazin-2-yl)-4-((2-(2-fluoropropan-2-yl)-6-methylpyrimidin-4-yl)amino)pyridin-2-yl)acetamide;
N-(5-(5,6-dihydro-8H-imidazo[2,1-c][1,4]oxazin-2-yl)-4-((2-(2-fluoropropan-2-yl)-6-methylpyrimidin-4-yl)amino)pyridin-2-yl)acetamide;
N-(5-([1,2,4]triazolo[4,3-a]pyrazin-6-yl)-4-((2-(2-fluoropropan-2-yl)-6-methylpyrimidin-4-yl)amino)pyridin-2-yl)acetamide;
N-(4-((2-(2-fluoropropan-2-yl)-6-methylpyrimidin-4-yl)amino)-5-(imidazo[1,2-a]pyrimidin-7-yl)pyridin-2-yl)acetamide;
N-(5-([1,2,4]triazolo[1,5-a]pyrimidin-5-yl)-4-((2-(2-fluoropropan-2-yl)-6-methylpyrimidin-4-yl)amino)pyridin-2-yl)acetamide;
N-(4-((2-(1,1-difluoroethyl)-6-methylpyrimidin-4-yl)amino)-5-(6,7-dihydro-5H-pyrazolo[5,1-b][1,3]oxazin-3-yl)pyridin-2-yl)acetamide;
N-(5-([1,2,4]triazolo[1,5-a]pyrimidin-5-yl)-4-((2-(1,1-difluoroethyl)-6-methylpyrimidin-4-yl)amino)pyridin-2-yl)acetamide;
N-(5-([1,2,4]triazolo[1,5-a]pyrimidin-5-yl)-4-((2-(1,1-difluoroethyl)pyrimidin-4-yl)amino)pyridin-2-yl)acetamide;
N-(4-((2-(2-fluoropropan-2-yl)-6-methylpyrimidin-4-yl)amino)-5-(pyrazolo[1,5-a]pyrimidin-5-yl)pyridin-2-yl)acetamide;
N-(4-((2-(1,1-difluoroethyl)pyrimidin-4-yl)amino)-5-(pyrazolo[1,5-a]pyrimidin-5-yl)pyridin-2-yl)acetamide;
N-(4-((2-(1,1-difluoroethyl)-6-methylpyrimidin-4-yl)amino)-5-(pyrazolo[1,5-a]pyrimidin-5-yl)pyridin-2-yl)acetamide;
N-(4-((2-(1,1-difluoroethyl)-6-methylpyrimidin-4-yl)amino)-5-(5-methyl-4-oxo-4,5,6,7-tetrahydropyrazolo[1,5-a]pyrazin-2-yl)pyridin-2-yl)acetamide;
N-(4-((2-(1,1-difluoroethyl)-6-methylpyrimidin-4-yl)amino)-5-(1-(ethylsulfonyl)-1H-pyrazol-3-yl)pyridin-2-yl)acetamide;
N-(4′-((2-(1,1-difluoroethyl)-6-methylpyrimidin-4-yl)amino)-5-(methylsulfonyl)-[2,3′-bipyridin]-6′-yl)acetamide;
N-(4-((2-(2-oxabicyclo[2.1.1]hexan-4-yl)-6-methylpyrimidin-4-yl)amino)-5-(5-ethyl-4,5,6,7-tetrahydropyrazolo[1,5-a]pyrazin-2-yl)pyridin-2-yl)acetamide;
N-(4-((2-(1,1-difluoroethyl)-6-methylpyrimidin-4-yl)amino)-5-(5-(methyl-d3)-4,5,6,7-tetrahydropyrazolo[1,5-a]pyrazin-2-yl)pyridin-2-yl)acetamide;
N-(4-((2-(1,1-difluoroethyl)-6-methylpyrimidin-4-yl)amino)-5-(6-(difluoromethyl)pyridazin-3-yl)pyridin-2-yl)acetamide;
(R)-N-(4-((6-methyl-2-(tetrahydrofuran-3-yl)pyrimidin-4-yl)amino)-5-(5-methyl-4,5,6,7-tetrahydropyrazolo[1,5-a]pyrazin-2-yl)pyridin-2-yl)acetamide;
(R)-N-(4-((2-(1,1-difluoroethyl)-6-methylpyrimidin-4-yl)amino)-5-(5,6-dimethyl-4,5,6,7-tetrahydropyrazolo[1,5-a]pyrazin-2-yl)pyridin-2-yl)acetamide;
N-(4-((2-(2-methoxyethoxy)pyrimidin-4-yl)amino)-5-(1-methyl-1H-pyrazol-3-yl)pyridin-2-yl)acetamide;
N-(4-((4-(2-methoxyethoxy)pyrimidin-2-yl)amino)-5-(1-methyl-1H-pyrazol-3-yl)pyridin-2-yl)acetamide;
N-(4-((2-methoxy-6-methylpyrimidin-4-yl)amino)-5-(1-methyl-1H-pyrazol-3-yl)pyridin-2-yl)acetamide;
N-(4-((2-(2-oxabicyclo[2.1.1]hexan-4-yl)-6-methylpyrimidin-4-yl)amino)-5-(1-methyl-1H-pyrazol-3-yl)pyridin-2-yl)acetamide;
N-(4-((4-methoxypyrimidin-2-yl)amino)-5-(1-methyl-1H-pyrazol-3-yl)pyridin-2-yl)acetamide;
N-(5-(1-methyl-1H-pyrazol-3-yl)-4-((6-methyl-2-(1-methyl-2-oxabicyclo[2.1.1]hexan-4-yl)pyrimidin-4-yl)amino)pyridin-2-yl)acetamide;
N-(5-(i-methyl-1H-pyrazol-3-yl)-4-((6-methyl-2-(tetrahydrofuran-3-yl)pyrimidin-4-yl)amino)pyridin-2-yl)acetamide;
N-(5-(1-methyl-1H-pyrazol-3-yl)-4-((2-(tetrahydrofuran-3-yl)pyrimidin-4-yl)amino)pyridin-2-yl)acetamide;
N-(4-((2-(2-oxabicyclo[2.2.1]heptan-4-yl)-6-methylpyrimidin-4-yl)amino)-5-(1-methyl-1H-pyrazol-3-yl)pyridin-2-yl)acetamide;
N-(4-((2-(1-fluorocyclopropyl)-6-methylpyrimidin-4-yl)amino)-5-(1-methyl-1H-pyrazol-3-yl)pyridin-2-yl)acetamide;
N-(4-((2-(2-methoxyethoxy)-6-methylpyrimidin-4-yl)amino)-5-(1-methyl-1H-pyrazol-3-yl)pyridin-2-yl)acetamide;
N-(4-((2-((1s,4s)-1-(fluoromethyl)-2-oxabicyclo[2.1.1]hexan-4-yl)-6-methylpyrimidin-4-yl)amino)-5-(1-methyl-1H-pyrazol-3-yl)pyridin-2-yl)acetamide;
N-(4-((2-(1-(methoxymethyl)-2-oxabicyclo[2.1.1]hexan-4-yl)-6-methylpyrimidin-4-yl)amino)-5-(1-methyl-1H-pyrazol-3-yl)pyridin-2-yl)acetamide;
N-(4-((2-(2-oxabicyclo[2.1.1]hexan-1-yl)-6-methylpyrimidin-4-yl)amino)-5-(1-methyl-1H-pyrazol-3-yl)pyridin-2-yl)acetamide;
N-(4-((2-(2-oxabicyclo[2.1.1]hexan-4-yl)-6-methylpyrimidin-4-yl)amino)-5-(2,2-dimethyl-2,3-dihydro-[1,4]dioxino[2,3-b]pyridin-6-yl)pyridin-2-yl)acetamide;
N-(4-((2-(2-oxabicyclo[2.1.1]hexan-4-yl)-6-methylpyrimidin-4-yl)amino)-5-(5,5-difluoro-5,6-dihydro-4H-pyrrolo[1,2-b]pyrazol-2-yl)pyridin-2-yl)acetamide;
N-(4′-((2-(2-oxabicyclo[2.1.1]hexan-4-yl)-6-methylpyrimidin-4-yl)amino)-5-(methoxymethyl)-[2,3′-bipyridin]-6′-yl)acetamide;
N-(4′-((2-(2-oxabicyclo[2.1.1]hexan-4-yl)-6-ethylpyrimidin-4-yl)amino)-5-(methoxymethyl)-[2,3′-bipyridin]-6′-yl)acetamide;
N-(5-(methoxymethyl)-4′-((2-(tetrahydrofuran-3-yl)pyrimidin-4-yl)amino)-[2,3′-bipyridin]-6′-yl)acetamide;
N-(5-(methoxymethyl)-4′-((6-methyl-2-(tetrahydrofuran-3-yl)pyrimidin-4-yl)amino)-[2,3′-bipyridin]-6′-yl)acetamide;
N-(4′-((6-ethyl-2-(tetrahydrofuran-3-yl)pyrimidin-4-yl)amino)-5-(methoxymethyl)-[2,3′-bipyridin]-6′-yl)acetamide;
N-(5-(methoxymethyl)-4′-((6-(methoxymethyl)-2-(tetrahydrofuran-3-yl)pyrimidin-4-yl)amino)-[2,3′-bipyridin]-6′-yl)acetamide;
N-(5-(methoxymethyl)-4′-((6-methyl-2-(tetrahydro-2H-pyran-3-yl)pyrimidin-4-yl)amino)-[2,3′-bipyridin]-6′-yl)acetamide;
N-(4′-((2-(2-oxabicyclo[2.1.1]hexan-4-yl)-6-methylpyrimidin-4-yl)amino)-5-(difluoromethyl)-[2,3′-bipyridin]-6′-yl)acetamide;
N-(4-((2-(1,1-difluoroethyl)pyrimidin-4-yl)amino)-5-(6,7-dihydro-4H-pyrano[4,3-d]thiazol-2-yl)pyridin-2-yl)acetamide;
N-(4′-((2-(2-oxabicyclo[2.1.1]hexan-4-yl)-6-methylpyrimidin-4-yl)amino)-5-morpholino-[2,3′-bipyridin]-6′-yl)acetamide;
N-(4′-((2-(2-oxabicyclo[2.1.1]hexan-4-yl)-6-methylpyrimidin-4-yl)amino)-5-(1-methoxyethyl)-[2,3′-bipyridin]-6′-yl)acetamide;
N-(4′-((2-(2-oxabicyclo[2.1.1]hexan-4-yl)-6-methylpyrimidin-4-yl)amino)-4-(difluoromethyl)-[2,3′-bipyridin]-6′-yl)acetamide;
N-(4′-((2-(2-oxabicyclo[2.1.1]hexan-4-yl)-6-methylpyrimidin-4-yl)amino)-5-(1-fluoroethyl)-[2,3′-bipyridin]-6′-yl)acetamide;
N-(4-((2-(1,1-difluoroethyl)-6-methylpyrimidin-4-yl)amino)-5-(2-methyloxazol-4-yl)pyridin-2-yl)acetamide;
N-(5-(1-cyclobutyl-1H-pyrazol-4-yl)-4-((2-(1,1-difluoroethyl)pyrimidin-4-yl)amino)pyridin-2-yl)acetamide;
N-(4′-((2-(1,1-difluoroethyl)pyrimidin-4-yl)amino)-5-fluoro-[2,3′-bipyridin]-6′-yl)acetamide;
N-(4-((2-(1,1-difluoroethyl)pyrimidin-4-yl)amino)-5-(2-methyloxazol-4-yl)pyridin-2-yl)acetamide;
N-(4′-((2-(1,1-difluoroethyl)-6-ethylpyrimidin-4-yl)amino)-5-fluoro-[2,3′-bipyridin]-6′-yl)acetamide;
N-(5-cyano-4′-((2-(1,1-difluoroethyl)-6-ethylpyrimidin-4-yl)amino)-[2,3′-bipyridin]-6′-yl)acetamide;
N-(4-((2-(1,1-difluoroethyl)-6-ethylpyrimidin-4-yl)amino)-5-(2-methyloxazol-4-yl)pyridin-2-yl)acetamide;
N-(4-((2-(1,1-difluoroethyl)-6-(2-methoxyethoxy)pyrimidin-4-yl)amino)-5-(pyrimidin-4-yl)pyridin-2-yl)acetamide;
N-(5-fluoro-4′-((6-methoxypyrazin-2-yl)amino)-[2,3′-bipyridin]-6′-yl)acetamide;
N-(4-((6-cyclopropoxy-2-(1,1-difluoroethyl)pyrimidin-4-yl)amino)-5-(2-(2-methoxyethyl)-2H-1,2,3-triazol-4-yl)pyridin-2-yl)acetamide;
N-(4′-((6-(difluoromethoxy)pyrazin-2-yl)amino)-5-(methoxymethyl)-[2,3′-bipyridin]-6′-yl)acetamide;
N-(4′-((2-(1,1-difluoroethyl)-5-methoxypyrimidin-4-yl)amino)-5-(methoxymethyl)-[2,3′-bipyridin]-6′-yl)acetamide;
N-(4-((2-(1,1-difluoroethyl)-5-methoxypyrimidin-4-yl)amino)-5-(1-methyl-1H-pyrazol-3-yl)pyridin-2-yl)acetamide;
N-(4′-((2-(1,1-difluoroethyl)-6-methylpyrimidin-4-yl)amino)-[2,3′-bipyridin]-6′-yl)acetamide;
N-(4-((2-(1,1-difluoroethyl)-6-methylpyrimidin-4-yl)amino)-5-(4-methyloxazol-2-yl)pyridin-2-yl)acetamide;
N-(4′-((2-(1,1-difluoroethyl)-6-methylpyrimidin-4-yl)amino)-5-methyl-[2,3′-bipyridin]-6′-yl)acetamide;
N-(4′-((2-(1,1-difluoroethyl)-6-methylpyrimidin-4-yl)amino)-5-fluoro-[2,3′-bipyridin]-6′-yl)acetamide;
N-(4-((2-(1,1-difluoroethyl)-6-methylpyrimidin-4-yl)amino)-5-(5-methylpyrimidin-2-yl)pyridin-2-yl)acetamide;
N-(4-((2-(1,1-difluoroethyl)-6-methylpyrimidin-4-yl)amino)-5-(oxazol-4-yl)pyridin-2-yl)acetamide;
N-(4-((2-(1,1-difluoroethyl)-6-methylpyrimidin-4-yl)amino)-5-(3-methyl-1H-pyrazol-1-yl)pyridin-2-yl)acetamide;
N-(4-((2-(1,1-difluoroethyl)-6-methylpyrimidin-4-yl)amino)-5-(5-fluoropyrazin-2-yl)pyridin-2-yl)acetamide;
N-(4′-((2-(1,1-difluoroethyl)-6-methylpyrimidin-4-yl)amino)-5-methoxy-[2,3′-bipyridin]-6′-yl)acetamide;
N-(4-((2-(1,1-difluoroethyl)-6-methylpyrimidin-4-yl)amino)-5-(2-methyloxazol-5-yl)pyridin-2-yl)acetamide;
N-(5-(2-cyano-1-methyl-1H-imidazol-4-yl)-4-((2-(1,1-difluoroethyl)-6-methylpyrimidin-4-yl)amino)pyridin-2-yl)acetamide;
N-(5-(1-cyclopropyl-1H-pyrazol-3-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(1-isopropyl-1H-pyrazol-3-yl)pyridin-2-yl)acetamide;
N-(5-(1-cyclobutyl-1H-pyrazol-4-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(thiazol-2-yl)pyridin-2-yl)acetamide;
N-(4-((2-(1,1-difluoroethyl)-6-methylpyrimidin-4-yl)amino)-5-(2-methylthiazol-4-yl)pyridin-2-yl)acetamide;
N-(4-((2-(1,1-difluoroethyl)-6-methylpyrimidin-4-yl)amino)-5-(1-methyl-1H-1,2,3-triazol-4-yl)pyridin-2-yl)acetamide;
N-(4-((2-(1,1-difluoroethyl)-6-methylpyrimidin-4-yl)amino)-5-(2-(methoxymethyl)-2H-1,2,3-triazol-4-yl)pyridin-2-yl)acetamide;
N-(4-((2-(1,1-difluoroethyl)-6-methylpyrimidin-4-yl)amino)-5-(5-methoxy-1-methyl-1H-1,2,4-triazol-3-yl)pyridin-2-yl)acetamide;
N-(4-((2-(1,1-difluoroethyl)-6-methylpyrimidin-4-yl)amino)-5-(2-isopropyl-2H-1,2,3-triazol-4-yl)pyridin-2-yl)acetamide;
N-(5-(5-(tert-butyl)-1,3,4-thiadiazol-2-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(2-isopropylthiazol-4-yl)pyridin-2-yl)acetamide;
N-(4-((2-(1,1-difluoroethyl)-6-methylpyrimidin-4-yl)amino)-5-(1-ethyl-1H-pyrazol-3-yl)pyridin-2-yl)acetamide;
N-(5-(1-(cyclopropylmethyl)-1H-pyrazol-3-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(1-propyl-1H-pyrazol-3-yl)pyridin-2-yl)acetamide;
N-(4-((2-(1,1-difluoroethyl)-6-methylpyrimidin-4-yl)amino)-5-(4-fluoro-1-methyl-1H-pyrazol-3-yl)pyridin-2-yl)acetamide;
N-(4-((2-(1,1-difluoroethyl)-6-methylpyrimidin-4-yl)amino)-5-(2-methoxythiazol-4-yl)pyridin-2-yl)acetamide;
N-(4-((2-(1,1-difluoroethyl)-6-methylpyrimidin-4-yl)amino)-5-(3-methyl-1,2,4-thiadiazol-5-yl)pyridin-2-yl)acetamide;
N-(4-((2-(1,1-difluoroethyl)-6-methylpyrimidin-4-yl)amino)-5-(1,5-dimethyl-1H-pyrazol-3-yl)pyridin-2-yl)acetamide;
N-(4-((2-(1,1-difluoroethyl)-6-methylpyrimidin-4-yl)amino)-5-(pyrazin-2-yl)pyridin-2-yl)acetamide;
N-(4-((2-(1,1-difluoroethyl)-6-methylpyrimidin-4-yl)amino)-5-(4-methoxy-6-methylpyrimidin-2-yl)pyridin-2-yl)acetamide;
N-(4′-((2-(1,1-difluoroethyl)-6-methylpyrimidin-4-yl)amino)-6-fluoro-5-methoxy-[2,3′-bipyridin]-6′-yl)acetamide;
N-(4′-((2-(1,1-difluoroethyl)-6-methylpyrimidin-4-yl)amino)-6-fluoro-[2,3′-bipyridin]-6′-yl)acetamide;
N-(4′-((2-(1,1-difluoroethyl)-6-methylpyrimidin-4-yl)amino)-4-fluoro-[2,3′-bipyridin]-6′-yl)acetamide;
N-(6′-bromo-4-((2-(1,1-difluoroethyl)-6-methylpyrimidin-4-yl)amino)-[3,3′-bipyridin]-6-yl)acetamide;
N-(5-bromo-4′-((2-(1,1-difluoroethyl)-6-methylpyrimidin-4-yl)amino)-[2,3′-bipyridin]-6′-yl)acetamide;
N-(4′-((2-(1,1-difluoroethyl)-6-methylpyrimidin-4-yl)amino)-4-(dimethylamino)-[2,3′-bipyridin]-6′-yl)acetamide;
N-(4′-((2-(1,1-difluoroethyl)-6-methylpyrimidin-4-yl)amino)-6-morpholino-[2,3′-bipyridin]-6′-yl)acetamide;
N-(4-((2-(1,1-difluoroethyl)-6-methylpyrimidin-4-yl)amino)-5-(2-(dimethylamino)pyrimidin-4-yl)pyridin-2-yl)acetamide;
N-(6-cyano-4′-((2-(1,1-difluoroethyl)-6-methylpyrimidin-4-yl)amino)-[2,3′-bipyridin]-6′-yl)acetamide;
N-(4-cyano-4′-((2-(1,1-difluoroethyl)-6-methylpyrimidin-4-yl)amino)-[2,3′-bipyridin]-6′-yl)acetamide;
N-(5-cyano-4′-((2-(1,1-difluoroethyl)-6-methylpyrimidin-4-yl)amino)-3-fluoro-[2,3′-bipyridin]-6′-yl)acetamide;
N-(6-cyano-4′-((2-(1,1-difluoroethyl)-6-methylpyrimidin-4-yl)amino)-5-fluoro-[2,3′-bipyridin]-6′-yl)acetamide;
N-(4′-((2-(1,1-difluoroethyl)-6-methylpyrimidin-4-yl)amino)-5-(methoxymethyl)-[2,3′-bipyridin]-6′-yl)acetamide;
N-(4′-((2-(1,1-difluoroethyl)-6-methylpyrimidin-4-yl)amino)-6-methoxy-[2,3′-bipyridin]-6′-yl)acetamide;
N-(4′-((2-(1,1-difluoroethyl)-6-methylpyrimidin-4-yl)amino)-6-(methoxymethyl)-[2,3′-bipyridin]-6′-yl)acetamide;
N-(4′-((2-(1,1-difluoroethyl)-6-methylpyrimidin-4-yl)amino)-5,6-dimethoxy-[2,3′-bipyridin]-6′-yl)acetamide;
(S)—N-(4′-((2-(1,1-difluoroethyl)-6-methylpyrimidin-4-yl)amino)-6-(1-hydroxyethyl)-[2,3′-bipyridin]-6′-yl)acetamide;
N-(4′-((2-(1,1-difluoroethyl)-6-methylpyrimidin-4-yl)amino)-6-(tetrahydrofuran-2-yl)-[2,3′-bipyridin]-6′-yl)acetamide;
(S)—N-(4′-((2-(1,1-difluoroethyl)-6-methylpyrimidin-4-yl)amino)-6-(4-methylmorpholin-2-yl)-[2,3′-bipyridin]-6′-yl)acetamide;
(S)—N-(4′-((2-(1,1-difluoroethyl)-6-methylpyrimidin-4-yl)amino)-6-(4-methylmorpholin-2-yl)-[2,3′-bipyridin]-6′-yl)acetamide;
(R)-N-(4′-((2-(1,1-difluoroethyl)-6-methylpyrimidin-4-yl)amino)-6-(4-methylmorpholin-2-yl)-[2,3′-bipyridin]-6′-yl)acetamide;
(R)-N-(4′-((2-(1,1-difluoroethyl)-6-methylpyrimidin-4-yl)amino)-5-(4-methylmorpholin-2-yl)-[2,3′-bipyridin]-6′-yl)acetamide;
(S)—N-(4′-((2-(1,1-difluoroethyl)-6-methylpyrimidin-4-yl)amino)-5-(4-methylmorpholin-2-yl)-[2,3′-bipyridin]-6′-yl)acetamide;
(R)-N-(4′-((2-(1,1-difluoroethyl)-6-methylpyrimidin-4-yl)amino)-5-(4-methylmorpholin-3-yl)-[2,3′-bipyridin]-6′-yl)acetamide;
(S)—N-(4′-((2-(1,1-difluoroethyl)-6-methylpyrimidin-4-yl)amino)-5-(4-methylmorpholin-3-yl)-[2,3′-bipyridin]-6′-yl)acetamide;
(S)—N-(4′-((2-(1,1-difluoroethyl)-6-methylpyrimidin-4-yl)amino)-6-(tetrahydro-2H-pyran-2-yl)-[2,3′-bipyridin]-6′-yl)acetamide;
N-(4′-((2-(1,1-difluoroethyl)-6-methylpyrimidin-4-yl)amino)-5-((dimethylamino)methyl)-[2,3′-bipyridin]-6′-yl)acetamide;
N-(4′-((2-(1,1-difluoroethyl)-6-methylpyrimidin-4-yl)amino)-5-(morpholinomethyl)-[2,3′-bipyridin]-6′-yl)acetamide;
N-(4′-((2-(1,1-difluoroethyl)-6-methylpyrimidin-4-yl)amino)-6-(morpholinomethyl)-[2,3′-bipyridin]-6′-yl)acetamide;
N-(4′-((2-(1,1-difluoroethyl)-6-methylpyrimidin-4-yl)amino)-5-(4-methylpiperazin-1-yl)-[2,3′-bipyridin]-6′-yl)acetamide;
N-(4′-((2-(1,1-difluoroethyl)-6-methylpyrimidin-4-yl)amino)-5-(dimethylamino)-[2,3′-bipyridin]-6′-yl)acetamide;
N-(6-(2-cyanopropan-2-yl)-4′-((2-(1,1-difluoroethyl)pyrimidin-4-yl)amino)-[2,3′-bipyridin]-6′-yl)acetamide;
N-(6-(2-cyanopropan-2-yl)-4′-((2-(1,1-difluoroethyl)-6-methylpyrimidin-4-yl)amino)-[2,3′-bipyridin]-6′-yl)acetamide;
N-(5-(5-cyanopyrimidin-2-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(5-methoxypyrimidin-2-yl)pyridin-2-yl)acetamide;
N-(5-(4-cyano-6-methylpyrimidin-2-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(4-methoxypyrimidin-2-yl)pyridin-2-yl)acetamide;
N-(4-((2-(1,1-difluoroethyl)-6-methylpyrimidin-4-yl)amino)-5-(5-fluoro-4-methoxypyrimidin-2-yl)pyridin-2-yl)acetamide;
N-(4-((2-(1,1-difluoroethyl)-6-methylpyrimidin-4-yl)amino)-5-(5-(difluoromethoxy)pyrimidin-2-yl)pyridin-2-yl)acetamide;
N-(4-((2-(1,1-difluoroethyl)-6-methylpyrimidin-4-yl)amino)-5-(2-(methoxymethyl)pyrimidin-4-yl)pyridin-2-yl)acetamide;
N-(4-((2-(1,1-difluoroethyl)-6-methylpyrimidin-4-yl)amino)-5-(5-((difluoromethoxy)methyl)pyrazin-2-yl)pyridin-2-yl)acetamide;
N-(4-((2-(1,1-difluoroethyl)-6-methylpyrimidin-4-yl)amino)-5-(4-methoxy-5-methylpyrimidin-2-yl)pyridin-2-yl)acetamide;
N-(4-((2-(1,1-difluoroethyl)-6-methylpyrimidin-4-yl)amino)-5-(6-(2-methoxyethyl)pyrazin-2-yl)pyridin-2-yl)acetamide;
N-(4-((2-(1,1-difluoroethyl)-6-methylpyrimidin-4-yl)amino)-5-(6-(methoxymethyl)pyrimidin-4-yl)pyridin-2-yl)acetamide;
N-(5-(4-cyclopropylpyrimidin-2-yl)-4-((2-(1,1-difluoroethyl)-6-methylpyrimidin-4-yl)amino)pyridin-2-yl)acetamide;
N-(5-(6-cyclopropylpyrazin-2-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(pyrimidin-2-yl)pyridin-2-yl)acetamide;
N-(5-(5-chloropyrimidin-2-yl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(5-(difluoromethyl)pyrimidin-2-yl)pyridin-2-yl)acetamide;
N-(4-((2-(1,1-difluoroethyl)-6-methylpyrimidin-4-yl)amino)-5-(4-methylpyrimidin-2-yl)pyridin-2-yl)acetamide;
N-(4-((2-(1,1-difluoroethyl)-6-methylpyrimidin-4-yl)amino)-5-(5-(methoxymethyl)pyrimidin-2-yl)pyridin-2-yl)acetamide;
N-(4-((2-(1,1-difluoroethyl)-6-methylpyrimidin-4-yl)amino)-5-(5-fluoro-4-methylpyrimidin-2-yl)pyridin-2-yl)acetamide;
N-(4-((2-(1,1-difluoroethyl)-6-methylpyrimidin-4-yl)amino)-5-(4-morpholinopyrimidin-2-yl)pyridin-2-yl)acetamide;
N-(4-((2-(1,1-difluoroethyl)-6-methylpyrimidin-4-yl)amino)-5-(6-((dimethylamino)methyl)pyrazin-2-yl)pyridin-2-yl)acetamide;
N-(4-((2-(1,1-difluoroethyl)-6-methylpyrimidin-4-yl)amino)-5-(6-methoxypyridazin-3-yl)pyridin-2-yl)acetamide;
N-(4-((2-(1,1-difluoroethyl)-6-methylpyrimidin-4-yl)amino)-5-(5-oxo-6,7-dihydro-5H-cyclopenta[b]pyridin-2-yl)pyridin-2-yl)acetamide;
N-(4-((2-(1,1-difluoroethyl)-6-methylpyrimidin-4-yl)amino)-5-(5,7-dihydrofuro[3,4-d]pyrimidin-2-yl)pyridin-2-yl)acetamide;
N-(4-((2-(1,1-difluoroethyl)-6-methylpyrimidin-4-yl)amino)-5-(7,8-dihydro-5H-pyrano[4,3-b]pyridin-2-yl)pyridin-2-yl)acetamide;
N-(4-((2-(1,1-difluoroethyl)-6-methylpyrimidin-4-yl)amino)-5-(imidazo[1,2-b]pyridazin-6-yl)pyridin-2-yl)acetamide;
N-(4-((2-(1,1-difluoroethyl)-6-methylpyrimidin-4-yl)amino)-5-(imidazo[1,2-a]pyrazin-6-yl)pyridin-2-yl)acetamide;
N-(4-((2-(1,1-difluoroethyl)-6-methylpyrimidin-4-yl)amino)-5-(7-oxo-5,7-dihydrofuro[3,4-d]pyrimidin-2-yl)pyridin-2-yl)acetamide;
(R)-N-(4-((2-(1,1-difluoroethyl)-6-methylpyrimidin-4-yl)amino)-5-(4,5-dimethyl-4,5,6,7-tetrahydropyrazolo[1,5-a]pyrazin-2-yl)pyridin-2-yl)acetamide;
N-(4-((2-(1,1-difluoroethyl)-6-methylpyrimidin-4-yl)amino)-5-(5-methyl-5,6-dihydro-4H-pyrrolo[3,4-d]thiazol-2-yl)pyridin-2-yl)acetamide;
N-(4-((2-(1,1-difluoroethyl)pyrimidin-4-yl)amino)-5-(pyrimidin-2-yl)pyridin-2-yl)acetamide;
N-(4-((2-(1,1-difluoroethyl)pyrimidin-4-yl)amino)-5-(5,6-dimethoxypyrazin-2-yl)pyridin-2-yl)acetamide;
N-(4-((2-(1,1-difluoroethyl)pyrimidin-4-yl)amino)-5-(2,6-dimethoxypyrimidin-4-yl)pyridin-2-yl)acetamide;
N-(5-(6-(cyanomethyl)pyrazin-2-yl)-4-((2-(1,1-difluoroethyl)pyrimidin-4-yl)amino)pyridin-2-yl)acetamide;
N-(4-((2-(1,1-difluoroethyl)pyrimidin-4-yl)amino)-5-(6-(difluoromethoxy)pyrazin-2-yl)pyridin-2-yl)acetamide;
N-(4-((2-(1,1-difluoroethyl)pyrimidin-4-yl)amino)-5-(6-(oxetan-3-yloxy)pyrazin-2-yl)pyridin-2-yl)acetamide;
N-(4-((2-(1,1-difluoroethyl)pyrimidin-4-yl)amino)-5-(6-(2-methoxyethoxy)pyrazin-2-yl)pyridin-2-yl)acetamide;
N-(4-((2-(1,1-difluoroethyl)pyrimidin-4-yl)amino)-5-(5-((difluoromethoxy)methyl)pyrazin-2-yl)pyridin-2-yl)acetamide;
N-(4-((2-(1,1-difluoroethyl)pyrimidin-4-yl)amino)-5-(5-methylpyrazin-2-yl)pyridin-2-yl)acetamide;
N-(5-(5-cyclopropylpyrazin-2-yl)-4-((2-(1,1-difluoroethyl)pyrimidin-4-yl)amino)pyridin-2-yl)acetamide;
N-(5-(5-(1,1-difluoroethyl)pyrazin-2-yl)-4-((2-(1,1-difluoroethyl)pyrimidin-4-yl)amino)pyridin-2-yl)acetamide;
N-(4-((2-(1,1-difluoroethyl)pyrimidin-4-yl)amino)-5-(2-(methoxymethyl)pyrimidin-4-yl)pyridin-2-yl)acetamide;
N-(5-(5-chloropyrimidin-2-yl)-4-((2-(1,1-difluoroethyl)pyrimidin-4-yl)amino)pyridin-2-yl)acetamide;
N-(4′-((2-(1,1-difluoroethyl)pyrimidin-4-yl)amino)-3-fluoro-[2,3′-bipyridin]-6′-yl)acetamide;
N-(4′-((2-(1,1-difluoroethyl)pyrimidin-4-yl)amino)-5-fluoro-4-methoxy-[2,3′-bipyridin]-6′-yl)acetamide;
N-(4′-((2-(1,1-difluoroethyl)pyrimidin-4-yl)amino)-4-methyl-[2,3′-bipyridin]-6′-yl)acetamide;
N-(5-cyano-4′-((2-(1,1-difluoroethyl)pyrimidin-4-yl)amino)-4-methyl-[2,3′-bipyridin]-6′-yl)acetamide;
N-(5-((6-oxa-3-azabicyclo[3.1.1]heptan-3-yl)methyl)-4′-((2-(1,1-difluoroethyl)pyrimidin-4-yl)amino)-[2,3′-bipyridin]-6′-yl)acetamide;
N-(5-((2-oxa-6-azaspiro[3.4]octan-6-yl)methyl)-4′-((2-(1,1-difluoroethyl)pyrimidin-4-yl)amino)-[2,3′-bipyridin]-6′-yl)acetamide;
N-(5-(1-(cyclopropylmethyl)-1H-pyrazol-3-yl)-4-((2-(1,1-difluoroethyl)pyrimidin-4-yl)amino)pyridin-2-yl)acetamide;
N-(4-((2-(1,1-difluoroethyl)pyrimidin-4-yl)amino)-5-(1-propyl-1H-pyrazol-3-yl)pyridin-2-yl)acetamide;
N-(4-((2-(1,1-difluoroethyl)pyrimidin-4-yl)amino)-5-(1-ethyl-1H-pyrazol-3-yl)pyridin-2-yl)acetamide;
N-(5-(2-cyano-1-methyl-1H-imidazol-4-yl)-4-((2-(1,1-difluoroethyl)pyrimidin-4-yl)amino)pyridin-2-yl)acetamide;
N-(4-((2-(1,1-difluoroethyl)pyrimidin-4-yl)amino)-5-(2-isopropyl-2H-1,2,3-triazol-4-yl)pyridin-2-yl)acetamide;
N-(4-((2-(1,1-difluoroethyl)pyrimidin-4-yl)amino)-5-(2-ethyl-2H-1,2,3-triazol-4-yl)pyridin-2-yl)acetamide;
N-(4-((2-(1,1-difluoroethyl)pyrimidin-4-yl)amino)-5-(2-(2-methoxyethyl)-2H-1,2,3-triazol-4-yl)pyridin-2-yl)acetamide;
N-(4-((2-(1,1-difluoroethyl)pyrimidin-4-yl)amino)-5-(2-(methoxymethyl)-2H-1,2,3-triazol-4-yl)pyridin-2-yl)acetamide;
N-(4-((2-(1,1-difluoroethyl)pyrimidin-4-yl)amino)-5-(2-methoxythiazol-4-yl)pyridin-2-yl)acetamide;
N-(4-((2-(1,1-difluoroethyl)pyrimidin-4-yl)amino)-5-(2-(2-fluoropropan-2-yl)thiazol-4-yl)pyridin-2-yl)acetamide;
N-(4-((2-(1,1-difluoroethyl)pyrimidin-4-yl)amino)-5-(5-ethyl-1,3,4-thiadiazol-2-yl)pyridin-2-yl)acetamide;
N-(5-(5-(tert-butyl)-1,3,4-thiadiazol-2-yl)-4-((2-(1,1-difluoroethyl)pyrimidin-4-yl)amino)pyridin-2-yl)acetamide;
N-(4-((2-(1,1-difluoroethyl)pyrimidin-4-yl)amino)-5-(3-methyl-1,2,4-thiadiazol-5-yl)pyridin-2-yl)acetamide;
N-(5-(5-cyclopropyl-1,3,4-oxadiazol-2-yl)-4-((2-(1,1-difluoroethyl)pyrimidin-4-yl)amino)pyridin-2-yl)acetamide;
N-(4-((2-(1,1-difluoroethyl)pyrimidin-4-yl)amino)-5-(5-oxo-6,7-dihydro-5H-cyclopenta[b]pyridin-2-yl)pyridin-2-yl)acetamide;
N-(4-((2-(1,1-difluoroethyl)-6-ethylpyrimidin-4-yl)amino)-5-(pyrimidin-2-yl)pyridin-2-yl)acetamide;
N-(4-((2-(1,1-difluoroethyl)-6-ethylpyrimidin-4-yl)amino)-5-(2-(methoxymethyl)pyrimidin-4-yl)pyridin-2-yl)acetamide;
N-(4-((2-(1,1-difluoroethyl)-6-ethylpyrimidin-4-yl)amino)-5-(5-methyl-4,5,6,7-tetrahydropyrazolo[1,5-a]pyrazin-2-yl)pyridin-2-yl)acetamide;
N-(4′-((5-(2-fluoropropan-2-yl)pyridin-3-yl)amino)-5-(2-hydroxypropan-2-yl)-[2,3′-bipyridin]-6′-yl)acetamide;
N-(4-((2-(2-fluoropropan-2-yl)-6-methylpyrimidin-4-yl)amino)-5-(5-fluoropyrimidin-2-yl)pyridin-2-yl)acetamide;
N-(4-((2-(1,1-difluoroethyl)pyrimidin-4-yl)amino)pyridin-2-yl)acetamide;
N-(4-((2-(1,1-difluoroethyl)-6-methylpyrimidin-4-yl)amino)-5-(dimethylamino)pyridin-2-yl)acetamide;
N-(4-((2-(1,1-difluoroethyl)-6-methylpyrimidin-4-yl)amino)-5-(pyrrolidin-1-yl)pyridin-2-yl)acetamide;
N-(4-((2-(1,1-difluoroethyl)-6-methylpyrimidin-4-yl)amino)-5-morpholinopyridin-2-yl)acetamide;
N-(4-((2-(1,1-difluoroethyl)pyrimidin-4-yl)amino)-5-(4-methyl-1H-pyrazol-1-yl)pyridin-2-yl)acetamide;
N-(4-((2-(1,1-difluoroethyl)-6-methylpyrimidin-4-yl)amino)-5-(4-methyl-1H-pyrazol-1-yl)pyridin-2-yl)acetamide;
N-(4-((2-(1,1-difluoroethyl)pyrimidin-4-yl)amino)-5-(4-methoxy-1H-pyrazol-1-yl)pyridin-2-yl)acetamide;
N-(4-((2-(1,1-difluoroethyl)-6-methylpyrimidin-4-yl)amino)-5-(4-methoxy-1H-pyrazol-1-yl)pyridin-2-yl)acetamide;
N-(5-(4-cyano-1H-pyrazol-1-yl)-4-((2-(1,1-difluoroethyl)pyrimidin-4-yl)amino)pyridin-2-yl)acetamide;
N-(5-(4-cyano-1H-pyrazol-1-yl)-4-((2-(1,1-difluoroethyl)-6-methylpyrimidin-4-yl)amino)pyridin-2-yl)acetamide;
N-(4-((2-(1,1-difluoroethyl)pyrimidin-4-yl)amino)-5-(3-methyl-1H-1,2,4-triazol-1-yl)pyridin-2-yl)acetamide;
N-(4-((2-(1,1-difluoroethyl)-6-methylpyrimidin-4-yl)amino)-5-(3-methyl-1H-1,2,4-triazol-1-yl)pyridin-2-yl)acetamide;
N-(5-(5-(cyanomethyl)-4,5,6,7-tetrahydropyrazolo[1,5-a]pyrazin-2-yl)-4-((2-(1,1-difluoroethyl)pyrimidin-4-yl)amino)pyridin-2-yl)acetamide;
N-(4-((2-(1,1-difluoroethyl)-6-methylpyrimidin-4-yl)amino)-5-(5-((dimethylamino)methyl)thiazol-2-yl)pyridin-2-yl)acetamide;
N-(4-((2-(1,1-difluoroethyl)-6-methylpyrimidin-4-yl)amino)-5-(5-(pyrrolidin-1-ylmethyl)thiazol-2-yl)pyridin-2-yl)acetamide;
N-(4-((2-(1,1-difluoroethyl)-6-methylpyrimidin-4-yl)amino)-5-(1-methyl-5-(morpholinomethyl)-1H-pyrazol-3-yl)pyridin-2-yl)acetamide;
N-(4′-((2-(1,1-difluoroethyl)-6-methylpyrimidin-4-yl)amino)-4-((dimethylamino)methyl)-[2,3′-bipyridin]-6′-yl)acetamide;
(R)-N-(4-((2-(1,1-difluoroethyl)-6-methylpyrimidin-4-yl)amino)-5-(5-(1-(dimethylamino)ethyl)pyrazin-2-yl)pyridin-2-yl)acetamide;
(R)-N-(4-((2-(1,1-difluoroethyl)-6-methylpyrimidin-4-yl)amino)-5-(5-(1-(pyrrolidin-1-yl)ethyl)pyrazin-2-yl)pyridin-2-yl)acetamide;
(S)—N-(4′-((2-(1,1-difluoroethyl)-6-methylpyrimidin-4-yl)amino)-4-(1-(dimethylamino)ethyl)-[2,3′-bipyridin]-6′-yl)acetamide;
N-(4-((2-(1,1-difluoroethyl)-6-methylpyrimidin-4-yl)amino)-5-(1,3,4-thiadiazol-2-yl)pyridin-2-yl)acetamide;
N-(4-((2-(1,1-difluoroethyl)-6-methylpyrimidin-4-yl)amino)-5-(6-(2-methoxyethyl)pyrimidin-4-yl)pyridin-2-yl)acetamide;
N-(4-((2-(2-oxabicyclo[2.1.1]hexan-4-yl)-6-methylpyrimidin-4-yl)amino)-5-(6-methylpyrimidin-4-yl)pyridin-2-yl)acetamide;
N-(4-((2-(2-oxabicyclo[2.1.1]hexan-4-yl)-6-methylpyrimidin-4-yl)amino)-5-(2-methylpyrimidin-4-yl)pyridin-2-yl)acetamide;
N-(4-((2-(2-oxabicyclo[2.1.1]hexan-4-yl)-6-methylpyrimidin-4-yl)amino)-5-(pyrimidin-4-yl)pyridin-2-yl)acetamide;
N-(4-((2-(2-oxabicyclo[2.1.1]hexan-4-yl)-6-methylpyrimidin-4-yl)amino)-5-(6-methoxypyridazin-3-yl)pyridin-2-yl)acetamide;
N-(4-((2-(2-oxabicyclo[2.1.1]hexan-4-yl)-6-methylpyrimidin-4-yl)amino)-5-(6-methoxypyrimidin-4-yl)pyridin-2-yl)acetamide;
N-(4′-((2-(2-oxabicyclo[2.1.1]hexan-4-yl)-6-methylpyrimidin-4-yl)amino)-5-cyano-[2,3′-bipyridin]-6′-yl)acetamide;
N-(4′-((2-(2-oxabicyclo[2.1.1]hexan-4-yl)-6-methylpyrimidin-4-yl)amino)-5-fluoro-[2,3′-bipyridin]-6′-yl)acetamide;
N-(4-((2-(1,1-difluoroethyl)-6-methylpyrimidin-4-yl)amino)-5-(5-methoxypyridazin-3-yl)pyridin-2-yl)acetamide;
N-(4-((2-(1,1-difluoroethyl)-6-methylpyrimidin-4-yl)amino)-5-(5-(2-methoxyethyl)-1,3,4-thiadiazol-2-yl)pyridin-2-yl)acetamide;
N-(4-((2-(1,1-difluoroethyl)-6-methylpyrimidin-4-yl)amino)-5-(5-(methoxymethyl)-1,3,4-thiadiazol-2-yl)pyridin-2-yl)acetamide;
N-(4-((2-(1,1-difluoroethyl)-6-methylpyrimidin-4-yl)amino)-5-(5-(2-methoxyethyl)thiazol-2-yl)pyridin-2-yl)acetamide;
N-(4-((2-(1,1-difluoroethyl)-6-methylpyrimidin-4-yl)amino)-5-(5-(methoxymethyl)-1-methyl-1H-pyrazol-3-yl)pyridin-2-yl)acetamide;
N-(4-((2-(1,1-difluoroethyl)-6-methylpyrimidin-4-yl)amino)-5-(5-(2-methoxypropan-2-yl)-1-methyl-1H-pyrazol-3-yl)pyridin-2-yl)acetamide;
N-(4-((2-(1,1-difluoroethyl)-6-methylpyrimidin-4-yl)amino)-5-(7-methyl-5,6,7,8-tetrahydro-[1,2,4]triazolo[1,5-a]pyrazin-2-yl)pyridin-2-yl)acetamide;
N-(4-((2-(1,1-difluoroethyl)-6-methylpyrimidin-4-yl)amino)-5-(5,6-dihydro-8H-[1,2,4]triazolo[5,1-c][1,4]oxazin-2-yl)pyridin-2-yl)acetamide;
N-(4-((2-(1,1-difluoroethyl)-6-methylpyrimidin-4-yl)amino)-5-(isothiazol-3-yl)pyridin-2-yl)acetamide;
N-(4-((2-(1,1-difluoroethyl)-6-methylpyrimidin-4-yl)amino)-5-(3-(methoxymethyl)-1,2,4-thiadiazol-5-yl)pyridin-2-yl)acetamide;
N-(4-((2-(1,1-difluoroethyl)-6-methylpyrimidin-4-yl)amino)-5-(5-(dimethylamino)-1,2,4-thiadiazol-3-yl)pyridin-2-yl)acetamide;
N-(4-((2-(1,1-difluoroethyl)-6-methylpyrimidin-4-yl)amino)-5-(4-(2-methoxyethyl)-1H-pyrazol-1-yl)pyridin-2-yl)acetamide;
N-(4-((2-(1,1-difluoroethyl)-6-methylpyrimidin-4-yl)amino)-5-(4-(methoxymethyl)-1H-pyrazol-1-yl)pyridin-2-yl)acetamide;
N-(5-(6-cyanopyrazin-2-yl)-4-((2-(1,1-difluoroethyl)pyrimidin-4-yl)amino)pyridin-2-yl)acetamide;
N-(4-((2-(1,1-difluoroethyl)pyrimidin-4-yl)amino)-5-(6-(difluoromethoxy)pyrimidin-4-yl)pyridin-2-yl)acetamide;
N-(4-((2-(1,1-difluoroethyl)-6-methylpyrimidin-4-yl)amino)-5-(5,6-dihydro-8H-imidazo[2,1-c][1,4]oxazin-2-yl)pyridin-2-yl)acetamide;
N-(4-((2-(1,1-difluoroethyl)-6-methylpyrimidin-4-yl)amino)-5-(6,7-dihydro-5H-pyrrolo[1,2-a]imidazol-2-yl)pyridin-2-yl)acetamide;
N-(4-((2-(1,1-difluoroethyl)-6-methylpyrimidin-4-yl)amino)-5-(2,3-dihydroimidazo[2,1-b]oxazol-6-yl)pyridin-2-yl)acetamide;
N-(4-((2-(1,1-difluoroethyl)-6-methylpyrimidin-4-yl)amino)-5-(2-(4-methylpiperazin-1-yl)thiazol-4-yl)pyridin-2-yl)acetamide;
N-(4-((2-(1,1-difluoroethyl)-6-methylpyrimidin-4-yl)amino)-5-(2-((2-methoxyethyl)(methyl)amino)thiazol-4-yl)pyridin-2-yl)acetamide;
N-(4-((2-(1,1-difluoroethyl)pyrimidin-4-yl)amino)-5-(5-((2-methoxyethyl)(methyl)amino)-1,3,4-thiadiazol-2-yl)pyridin-2-yl)acetamide;
N-(4-((2-(1,1-difluoroethyl)pyrimidin-4-yl)amino)-5-(5-(4-methylpiperazin-1-yl)-1,3,4-thiadiazol-2-yl)pyridin-2-yl)acetamide;
N-(4-((2-(1,1-difluoroethyl)pyrimidin-4-yl)amino)-5-(2-(difluoromethyl)pyrimidin-4-yl)pyridin-2-yl)acetamide;
N-(4-((2-(1,1-difluoroethyl)-6-methylpyrimidin-4-yl)amino)-5-(2-(2-methoxyethoxy)pyrimidin-4-yl)pyridin-2-yl)acetamide;
N-(4-((2-(1,1-difluoroethyl)-6-methylpyrimidin-4-yl)amino)-5-(4-(2-methoxyethoxy)pyrimidin-2-yl)pyridin-2-yl)acetamide;
N-(4-((2-(1,1-difluoroethyl)pyrimidin-4-yl)amino)-5-(2-(2-morpholinoethyl)-2H-1,2,3-triazol-4-yl)pyridin-2-yl)acetamide;
N-(4-((2-(1,1-difluoroethyl)pyrimidin-4-yl)amino)-5-(2-(2-(4-methylpiperazin-1-yl)ethyl)-2H-1,2,3-triazol-4-yl)pyridin-2-yl)acetamide;
N-(4-((2-(1,1-difluoroethyl)pyrimidin-4-yl)amino)-5-(5,6,7,8-tetrahydroimidazo[1,2-a]pyridin-2-yl)pyridin-2-yl)acetamide;
N-(4-((2-(1,1-difluoroethyl)pyrimidin-4-yl)amino)-5-(5-(methoxymethyl)thiazol-2-yl)pyridin-2-yl)acetamide;
N-(4-((2-(1,1-difluoroethyl)pyrimidin-4-yl)amino)-5-(5-((dimethylamino)methyl)thiazol-2-yl)pyridin-2-yl)acetamide;
N-(4-((2-(1,1-difluoroethyl)-6-methylpyrimidin-4-yl)amino)-5-(5-((4-methylpiperazin-1-yl)methyl)thiazol-2-yl)pyridin-2-yl)acetamide;
N-(4-((2-(1,1-difluoroethyl)-6-methylpyrimidin-4-yl)amino)-5-(4-((dimethylamino)methyl)thiazol-2-yl)pyridin-2-yl)acetamide;
N-(4-((2-(1,1-difluoroethyl)-6-methylpyrimidin-4-yl)amino)-5-(4-((4-methylpiperazin-1-yl)methyl)thiazol-2-yl)pyridin-2-yl)acetamide;
N-(4-((2-(1,1-difluoroethyl)-6-methylpyrimidin-4-yl)amino)-5-(2-((dimethylamino)methyl)thiazol-4-yl)pyridin-2-yl)acetamide;
N-(4-((2-(1,1-difluoroethyl)-6-methylpyrimidin-4-yl)amino)-5-(2-((4-methylpiperazin-1-yl)methyl)thiazol-4-yl)pyridin-2-yl)acetamide;
N-(5-(2-(azetidin-1-yl)thiazol-4-yl)-4-((2-(1,1-difluoroethyl)pyrimidin-4-yl)amino)pyridin-2-yl)acetamide;
N-(5-(2-(2-oxa-6-azaspiro[3.3]heptan-6-yl)thiazol-4-yl)-4-((2-(1,1-difluoroethyl)pyrimidin-4-yl)amino)pyridin-2-yl)acetamide;
N-(5-(2-cyanothiazol-4-yl)-4-((2-(1,1-difluoroethyl)pyrimidin-4-yl)amino)pyridin-2-yl)acetamide;
N-(4-((2-(1,1-difluoroethyl)-6-methylpyrimidin-4-yl)amino)-5-(5-(pyrrolidin-1-ylmethyl)pyrazin-2-yl)pyridin-2-yl)acetamide;
N-(4-((2-(1,1-difluoroethyl)-6-methylpyrimidin-4-yl)amino)-5-(5-((4-methylpiperazin-1-yl)methyl)pyrazin-2-yl)pyridin-2-yl)acetamide;
N-(4-((2-(1,1-difluoroethyl)-6-methylpyrimidin-4-yl)amino)-5-(5-(morpholinomethyl)pyrazin-2-yl)pyridin-2-yl)acetamide;
1-(4-((2-(1,1-difluoroethyl)pyrimidin-4-yl)amino)-5-(5-methyl-4,5,6,7-tetrahydropyrazolo[1,5-a]pyrazin-2-yl)pyridin-2-yl)-3-methylurea;
1-(4-((2-(1,1-difluoroethyl)pyrimidin-4-yl)amino)-5-(5-methyl-4,5,6,7-tetrahydrothiazolo[5,4-c]pyridin-2-yl)pyridin-2-yl)-3-methylurea;
N-(4-((2-(1,1-difluoroethyl)pyrimidin-4-yl)amino)-5-(5-methyl-4,5,6,7-tetrahydrothiazolo[4,5-c]pyridin-2-yl)pyridin-2-yl)formamide;
N-(4-((2-(1,1-difluoroethyl)pyrimidin-4-yl)amino)-5-(5-methyl-4,5,6,7-tetrahydrothiazolo[4,5-c]pyridin-2-yl)pyridin-2-yl)acetamide;
N-(4-((2-(1,1-difluoroethyl)pyrimidin-4-yl)amino)-5-(5-methyl-4,5,6,7-tetrahydrothiazolo[5,4-c]pyridin-2-yl)pyridin-2-yl)acetamide;
N-(4-((2-(1,1-difluoroethyl)-6-ethylpyrimidin-4-yl)amino)-5-(pyrimidin-4-yl)pyridin-2-yl)acetamide;
N-(4-((2-(2-fluoropropan-2-yl)-6-methylpyrimidin-4-yl)amino)-5-(pyrimidin-4-yl)pyridin-2-yl)acetamide;
N-(4-((6-ethyl-2-(2-fluoropropan-2-yl)pyrimidin-4-yl)amino)-5-(pyrimidin-4-yl)pyridin-2-yl)acetamide;
N-(4-((2-(1,1-difluoroethyl)-6-methylpyrimidin-4-yl)amino)-5-(5-(2-(dimethylamino)propan-2-yl)-1-methyl-1H-pyrazol-3-yl)pyridin-2-yl)acetamide;
N-(4-((2-(1,1-difluoroethyl)-6-methylpyrimidin-4-yl)amino)-5-(1-methyl-5-(pyrrolidin-1-ylmethyl)-1H-pyrazol-3-yl)pyridin-2-yl)acetamide;
N-(4-((2-(1,1-difluoroethyl)-6-methylpyrimidin-4-yl)amino)-5-(1-methyl-5-((4-methylpiperazin-1-yl)methyl)-1H-pyrazol-3-yl)pyridin-2-yl)acetamide;
N-(4′-((2-(1,1-difluoroethyl)-6-methylpyrimidin-4-yl)amino)-4-(pyrrolidin-1-ylmethyl)-[2,3′-bipyridin]-6′-yl)acetamide;
N-(4′-((2-(1,1-difluoroethyl)-6-methylpyrimidin-4-yl)amino)-4-(morpholinomethyl)-[2,3′-bipyridin]-6′-yl)acetamide;
N-(4-((2-(1,1-difluoroethyl)-6-methylpyrimidin-4-yl)amino)-5-(5-((dimethylamino)methyl)pyrazin-2-yl)pyridin-2-yl)acetamide;
N-(4-((2-(1,1-difluoroethyl)-6-methoxypyrimidin-4-yl)amino)pyridin-2-yl)acetamide;
N-(4-((2-(1,1-difluoroethyl)-6-(2-methoxyethoxy)pyrimidin-4-yl)amino)pyridin-2-yl)acetamide;
N-(5-((1R,2S)-2-cyanocyclopropyl)-4-((2-(1,1-difluoroethyl)-6-methylpyrimidin-4-yl)amino)pyridin-2-yl)acetamide;
N-(4-((2-(1,1-difluoroethyl)-6-methylpyrimidin-4-yl)amino)-5-(1-(difluoromethyl)-1H-pyrazol-3-yl)pyridin-2-yl)acetamide;
N-(4-((2-(1,1-difluoroethyl)-6-methylpyrimidin-4-yl)amino)-5-(1-methyl-1H-pyrazol-4-yl)pyridin-2-yl)acetamide;
N-(4-((2-(1,1-difluoroethyl)-6-methylpyrimidin-4-yl)amino)-5-(2,2-dimethyl-2,3-dihydro-[1,4]dioxino[2,3-b]pyridin-6-yl)pyridin-2-yl)acetamide;
N-(4-((2-(1,1-difluoroethyl)-6-methylpyrimidin-4-yl)amino)-5-(5-fluoro-1-methyl-1H-pyrazol-3-yl)pyridin-2-yl)acetamide;
N-(4-((2-(1,1-difluoroethyl)-6-methylpyrimidin-4-yl)amino)-5-(5-methoxypyrazin-2-yl)pyridin-2-yl)acetamide;
N-(4-((2-(1,1-difluoroethyl)-6-ethylpyrimidin-4-yl)amino)-5-fluoropyridin-2-yl)acetamide;
N-(4-((2-(1,1-difluoroethyl)-6-isopropylpyrimidin-4-yl)amino)-5-fluoropyridin-2-yl)acetamide;
N-(4-((6-cyclopropoxy-2-(1,1-difluoroethyl)pyrimidin-4-yl)amino)-5-fluoropyridin-2-yl)acetamide;
N-(4-((2-(1,1-difluoroethyl)-6-(1-isopropyl-1H-pyrazol-4-yl)pyrimidin-4-yl)amino)-5-fluoropyridin-2-yl)acetamide;
N-(4-((6-(1,1-difluoroethyl)pyridin-2-yl)amino)-5-(2,2-dimethyl-2,3-dihydro-[1,4]dioxino[2,3-b]pyridin-6-yl)pyridin-2-yl)acetamide;
N-(4-((2-(1,1-difluoroethyl)-6-ethylpyrimidin-4-yl)amino)-5-(2,2-dimethyl-2,3-dihydro-[1,4]dioxino[2,3-b]pyridin-6-yl)pyridin-2-yl)acetamide;
N-(4-((2-(1,1-difluoroethyl)-6-isopropylpyrimidin-4-yl)amino)-5-(2,2-dimethyl-2,3-dihydro-[1,4]dioxino[2,3-b]pyridin-6-yl)pyridin-2-yl)acetamide;
N-(4-((2-(1,1-difluoroethyl)-6-ethylpyrimidin-4-yl)amino)-5-(pyrazin-2-yl)pyridin-2-yl)acetamide;
N-(4-((6-cyclopropoxy-2-(1,1-difluoroethyl)pyrimidin-4-yl)amino)-5-(pyrazin-2-yl)pyridin-2-yl)acetamide;
N-(4-((2-(1,1-difluoroethyl)pyrimidin-4-yl)amino)-5-(5-(difluoromethoxy)pyrimidin-2-yl)pyridin-2-yl)acetamide;
N-(4′-((2-(1,1-difluoroethyl)pyrimidin-4-yl)amino)-5-(difluoromethoxy)-[2,3′-bipyridin]-6′-yl)acetamide;
N-(4′-((2-(1,1-difluoroethyl)-6-methylpyrimidin-4-yl)amino)-5-(difluoromethyl)-[2,3′-bipyridin]-6′-yl)acetamide;
N-(4-((2-(1,1-difluoroethyl)pyrimidin-4-yl)amino)-5-(5-methoxypyrazin-2-yl)pyridin-2-yl)acetamide;
N-(4-((2-(1,1-difluoroethyl)-6-ethylpyrimidin-4-yl)amino)-5-(5-methoxypyrazin-2-yl)pyridin-2-yl)acetamide;
N-(4-((6-cyclopropoxy-2-(1,1-difluoroethyl)pyrimidin-4-yl)amino)-5-(5-methoxypyrazin-2-yl)pyridin-2-yl)acetamide;
N-(4-((2-(1,1-difluoroethyl)-6-ethylpyrimidin-4-yl)amino)-5-(1-methyl-6-oxo-1,6-dihydropyridazin-3-yl)pyridin-2-yl)acetamide;
N-(4-((2-(1,1-difluoroethyl)-6-isopropylpyrimidin-4-yl)amino)-5-(6-methoxypyridazin-3-yl)pyridin-2-yl)acetamide;
N-(4-((6-cyclopropoxy-2-(1,1-difluoroethyl)pyrimidin-4-yl)amino)-5-(6-methoxypyridazin-3-yl)pyridin-2-yl)acetamide;
N-(4-((2-(1,1-difluoroethyl)-6-methylpyrimidin-4-yl)amino)-5-(5-(methoxymethyl)pyrazin-2-yl)pyridin-2-yl)acetamide;
N-(4-((2-(2-fluoropropan-2-yl)pyrimidin-4-yl)amino)-5-(6-methoxypyridazin-3-yl)pyridin-2-yl)acetamide;
N-(4-((2-(1,1-difluoroethyl)-6-ethylpyrimidin-4-yl)amino)-5-(5-(methoxymethyl)pyrazin-2-yl)pyridin-2-yl)acetamide;
N-(4-((2-(1,1-difluoroethyl)-6-methylpyrimidin-4-yl)amino)-5-(2-(1,1-difluoroethyl)pyrimidin-4-yl)pyridin-2-yl)acetamide;
N-(5-(5-cyanopyrazin-2-yl)-4-((2-(1,1-difluoroethyl)-6-methylpyrimidin-4-yl)amino)pyridin-2-yl)acetamide;
N-(4′-((2-(1,1-difluoroethyl)-6-ethylpyrimidin-4-yl)amino)-5-(2-hydroxypropan-2-yl)-[2,3′-bipyridin]-6′-yl)acetamide;
(R)-N-(5-(5-(1,4-dioxan-2-yl)pyrimidin-2-yl)-4-((2-(1,1-difluoroethyl)-6-ethylpyrimidin-4-yl)amino)pyridin-2-yl)acetamide;
N-(4-((2-(1,1-difluoroethyl)pyrimidin-4-yl)amino)-5-(6-(dimethylamino)pyridazin-3-yl)pyridin-2-yl)acetamide;
N-(4-((2-(1,1-difluoroethyl)pyrimidin-4-yl)amino)-5-(5-(dimethylamino)pyrazin-2-yl)pyridin-2-yl)acetamide;
N-(4-((2-(1,1-difluoroethyl)pyrimidin-4-yl)amino)-5-(5-(pyrrolidin-1-yl)pyrazin-2-yl)pyridin-2-yl)acetamide;
N-(4-((2-(1,1-difluoroethyl)pyrimidin-4-yl)amino)-5-(5-morpholinopyrazin-2-yl)pyridin-2-yl)acetamide;
N-(4-((2-(1,1-difluoroethyl)pyrimidin-4-yl)amino)-5-(6-(pyrrolidin-1-yl)pyridazin-3-yl)pyridin-2-yl)acetamide;
N-(4′-((2-(1,1-difluoroethyl)-6-ethylpyrimidin-4-yl)amino)-5-(methoxymethyl)-[2,3′-bipyridin]-6′-yl)acetamide;
N-(5-(5-cyanopyrimidin-2-yl)-4-((2-(1,1-difluoroethyl)-6-ethylpyrimidin-4-yl)amino)pyridin-2-yl)acetamide;
N-(4-((2-(1,1-difluoroethyl)-6-ethylpyrimidin-4-yl)amino)-5-(5-(difluoromethoxy)pyrimidin-2-yl)pyridin-2-yl)acetamide;
N-(4′-((2-(1,1-difluoroethyl)-6-ethylpyrimidin-4-yl)amino)-5-(difluoromethoxy)-[2,3′-bipyridin]-6′-yl)acetamide;
N-(5-(5-cyanopyrazin-2-yl)-4-((2-(1,1-difluoroethyl)pyrimidin-4-yl)amino)pyridin-2-yl)acetamide;
N-(4′-((2-(1,1-difluoroethyl)pyrimidin-4-yl)amino)-5-(methoxymethyl)-[2,3′-bipyridin]-6′-yl)acetamide;
N-(4′-((2-(1,1-difluoroethyl)pyrimidin-4-yl)amino)-5-(2-hydroxypropan-2-yl)-[2,3′-bipyridin]-6′-yl)acetamide;
N-(4-((2-(1,1-difluoroethyl)pyrimidin-4-yl)amino)-5-(5-(2-hydroxypropan-2-yl)pyrazin-2-yl)pyridin-2-yl)acetamide;
N-(4-((2-(1,1-difluoroethyl)pyrimidin-4-yl)amino)-5-(5-(difluoromethoxy)pyrazin-2-yl)pyridin-2-yl)acetamide;
N-(4-((2-(1,1-difluoroethyl)-6-ethylpyrimidin-4-yl)amino)-5-(5-(2-hydroxypropan-2-yl)pyrazin-2-yl)pyridin-2-yl)acetamide;
N-(4-((2-(1,1-difluoroethyl)-6-ethylpyrimidin-4-yl)amino)-5-(5-(dimethylamino)pyrazin-2-yl)pyridin-2-yl)acetamide;
N-(4′-((2-(1,1-difluoroethyl)pyrimidin-4-yl)amino)-5-(methylamino)-[2,3′-bipyridin]-6′-yl)acetamide;
N-(4-((2-(1,1-difluoroethyl)pyrimidin-4-yl)amino)-5-(6-(difluoromethoxy)pyridazin-3-yl)pyridin-2-yl)acetamide;
N-(4′-((2-(1,1-difluoroethyl)-6-ethylpyrimidin-4-yl)amino)-5-(methylamino)-[2,3′-bipyridin]-6′-yl)acetamide;
N-(4-((2-(1,1-difluoroethyl)-6-ethylpyrimidin-4-yl)amino)-5-(5-(methylamino)pyrazin-2-yl)pyridin-2-yl)acetamide;
N-(4-((2-(1,1-difluoroethyl)-6-ethylpyrimidin-4-yl)amino)-5-(5-morpholinopyrazin-2-yl)pyridin-2-yl)acetamide;
N-(4-((2-(1,1-difluoroethyl)-6-ethylpyrimidin-4-yl)amino)-5-(6-(dimethylamino)pyridazin-3-yl)pyridin-2-yl)acetamide;
N-(4-((2-(1,1-difluoroethyl)-6-ethylpyrimidin-4-yl)amino)-5-(6-morpholinopyridazin-3-yl)pyridin-2-yl)acetamide;
N-(5-(5-cyanopyrazin-2-yl)-4-((2-(1,1-difluoroethyl)-6-ethylpyrimidin-4-yl)amino)pyridin-2-yl)acetamide;
N-(4-((2-(1,1-difluoroethyl)-6-ethylpyrimidin-4-yl)amino)-5-(1-methyl-6-oxo-1,6-dihydropyridazin-3-yl)pyridin-2-yl)acetamide;
N-(4-((2-(1,1-difluoroethyl)pyrimidin-4-yl)amino)-5-(5-((2-methoxyethyl)(methyl)amino)pyrazin-2-yl)pyridin-2-yl)acetamide;
(R)-N-(4-((2-(1,1-difluoroethyl)pyrimidin-4-yl)amino)-5-(5-(3-methoxypyrrolidin-1-yl)pyrazin-2-yl)pyridin-2-yl)acetamide;
N-(4-((2-(1,1-difluoroethyl)pyrimidin-4-yl)amino)-5-(5-(3-methoxy-3-methylazetidin-1-yl)pyrazin-2-yl)pyridin-2-yl)acetamide;
N-(5-(5-(2-oxa-6-azaspiro[3.4]octan-6-yl)pyrazin-2-yl)-4-((2-(1,1-difluoroethyl)pyrimidin-4-yl)amino)pyridin-2-yl)acetamide;
N-(5-(5-((1S,4S)-2-oxa-5-azabicyclo[2.2.1]heptan-5-yl)pyrazin-2-yl)-4-((2-(1,1-difluoroethyl)pyrimidin-4-yl)amino)pyridin-2-yl)acetamide;
N-(5-(5-((1R,4R)-2-oxa-5-azabicyclo[2.2.1]heptan-5-yl)pyrazin-2-yl)-4-((2-(1,1-difluoroethyl)pyrimidin-4-yl)amino)pyridin-2-yl)acetamide;
N-(5-(5-(6-oxa-2-azaspiro[3.4]octan-2-yl)pyrazin-2-yl)-4-((2-(1,1-difluoroethyl)pyrimidin-4-yl)amino)pyridin-2-yl)acetamide;
(R)-N-(4-((2-(1,1-difluoroethyl)pyrimidin-4-yl)amino)-5-(6-(3-methoxypyrrolidin-1-yl)pyridazin-3-yl)pyridin-2-yl)acetamide;
N-(4-((2-(1,1-difluoroethyl)pyrimidin-4-yl)amino)-5-(6-(3-methoxy-3-methylazetidin-1-yl)pyridazin-3-yl)pyridin-2-yl)acetamide;
N-(5-(6-(2-oxa-6-azaspiro[3.3]heptan-6-yl)pyridazin-3-yl)-4-((2-(1,1-difluoroethyl)pyrimidin-4-yl)amino)pyridin-2-yl)acetamide;
N-(5-(6-((1S,4S)-2-oxa-5-azabicyclo[2.2.1]heptan-5-yl)pyridazin-3-yl)-4-((2-(1,1-difluoroethyl)pyrimidin-4-yl)amino)pyridin-2-yl)acetamide;
N-(5-(6-(6-oxa-2-azaspiro[3.4]octan-2-yl)pyridazin-3-yl)-4-((2-(1,1-difluoroethyl)pyrimidin-4-yl)amino)pyridin-2-yl)acetamide;
N-(4′-((2-(1,1-difluoroethyl)pyrimidin-4-yl)amino)-5-(pyrrolidin-1-ylmethyl)-[2,3′-bipyridin]-6′-yl)acetamide;
N-(4′-((2-(1,1-difluoroethyl)pyrimidin-4-yl)amino)-5-(((2-methoxyethyl)(methyl)amino)methyl)-[2,3′-bipyridin]-6′-yl)acetamide;
(R)-N-(4′-((2-(1,1-difluoroethyl)pyrimidin-4-yl)amino)-5-((3-methylmorpholino)methyl)-[2,3′-bipyridin]-6′-yl)acetamide;
(S)—N-(4′-((2-(1,1-difluoroethyl)pyrimidin-4-yl)amino)-5-((3-methoxypyrrolidin-1-yl)methyl)-[2,3′-bipyridin]-6′-yl)acetamide;
N-(4′-((2-(1,1-difluoroethyl)pyrimidin-4-yl)amino)-5-((3-methoxy-3-methylazetidin-1-yl)methyl)-[2,3′-bipyridin]-6′-yl)acetamide;
N-(4′-((2-(1,1-difluoroethyl)pyrimidin-4-yl)amino)-5-(((tetrahydro-2H-pyran-4-yl)amino)methyl)-[2,3′-bipyridin]-6′-yl)acetamide;
N-(5-((2-oxa-6-azaspiro[3.3]heptan-6-yl)methyl)-4′-((2-(1,1-difluoroethyl)pyrimidin-4-yl)amino)-[2,3′-bipyridin]-6′-yl)acetamide;
N-(5-(((1S,4S)-2-oxa-5-azabicyclo[2.2.1]heptan-5-yl)methyl)-4′-((2-(1,1-difluoroethyl)pyrimidin-4-yl)amino)-[2,3′-bipyridin]-6′-yl)acetamide;
N-(5-(((1R,4R)-2-oxa-5-azabicyclo[2.2.1]heptan-5-yl)methyl)-4′-((2-(1,1-difluoroethyl)pyrimidin-4-yl)amino)-[2,3′-bipyridin]-6′-yl)acetamide;
N-(5-(5-((1R,5S)-6-oxa-3-azabicyclo[3.1.1]heptan-3-yl)pyrazin-2-yl)-4-((2-(1,1-difluoroethyl)pyrimidin-4-yl)amino)pyridin-2-yl)acetamide;
N-(5-(5-(2-oxa-6-azaspiro[3.3]heptan-6-yl)pyrazin-2-yl)-4-((2-(1,1-difluoroethyl)pyrimidin-4-yl)amino)pyridin-2-yl)acetamide;
N-(5-((6-oxa-2-azaspiro[3.4]octan-2-yl)methyl)-4′-((2-(1,1-difluoroethyl)pyrimidin-4-yl)amino)-[2,3′-bipyridin]-6′-yl)acetamide;
N-(5-(6-(2-oxa-6-azaspiro[3.4]octan-6-yl)pyridazin-3-yl)-4-((2-(1,1-difluoroethyl)pyrimidin-4-yl)amino)pyridin-2-yl)acetamide;
N-(4-((2-(1,1-difluoroethyl)-6-ethylpyrimidin-4-yl)amino)-5-(6-(difluoromethyl)pyrimidin-4-yl)pyridin-2-yl)acetamide;
(S)—N-(4′-((2-(1,1-difluoroethyl)pyrimidin-4-yl)amino)-5-((3-methylmorpholino)methyl)-[2,3′-bipyridin]-6′-yl)acetamide;
N-(4-((2-(1,1-difluoroethyl)-6-ethylpyrimidin-4-yl)amino)-5-(5-(pyrrolidin-1-ylmethyl)pyrazin-2-yl)pyridin-2-yl)acetamide;
N-(5-(5-(((1S,4S)-2-oxa-5-azabicyclo[2.2.1]heptan-5-yl)methyl)pyrazin-2-yl)-4-((2-(1,1-difluoroethyl)-6-ethylpyrimidin-4-yl)amino)pyridin-2-yl)acetamide;
N-(4-((2-(1,1-difluoroethyl)-6-ethylpyrimidin-4-yl)amino)-5-(5-((dimethylamino)methyl)pyrazin-2-yl)pyridin-2-yl)acetamide;
N-(4-((2-(1,1-difluoroethyl)pyrimidin-4-yl)amino)-5-(5-(2-methoxypropan-2-yl)pyrazin-2-yl)pyridin-2-yl)acetamide;
N-(5-(6-((1R,4R)-2-oxa-5-azabicyclo[2.2.1]heptan-5-yl)pyridazin-3-yl)-4-((2-(1,1-difluoroethyl)pyrimidin-4-yl)amino)pyridin-2-yl)acetamide;
(R)-N-(5-(5-(1,4-dioxan-2-yl)pyrazin-2-yl)-4-((2-(1,1-difluoroethyl)-6-ethylpyrimidin-4-yl)amino)pyridin-2-yl)acetamide;
N-(4-((2-(1,1-difluoroethyl)-6-ethylpyrimidin-4-yl)amino)-5-(5-(morpholinomethyl)pyrazin-2-yl)pyridin-2-yl)acetamide;
N-(5-(5-(((1R,5S)-6-oxa-3-azabicyclo[3.1.1]heptan-3-yl)methyl)pyrazin-2-yl)-4-((2-(1,1-difluoroethyl)-6-ethylpyrimidin-4-yl)amino)pyridin-2-yl)acetamide;
N-(4′-((2-(1,1-difluoroethyl)-6-ethylpyrimidin-4-yl)amino)-5-(pyrrolidin-1-ylmethyl)-[2,3′-bipyridin]-6′-yl)acetamide;
N-(5-(((1S,4S)-2-oxa-5-azabicyclo[2.2.1]heptan-5-yl)methyl)-4′-((2-(1,1-difluoroethyl)-6-ethylpyrimidin-4-yl)amino)-[2,3′-bipyridin]-6′-yl)acetamide;
N-(4-((2-(1,1-difluoroethyl)-6-ethylpyrimidin-4-yl)amino)-5-(2-(pyrrolidin-1-ylmethyl)pyrimidin-4-yl)pyridin-2-yl)acetamide;
N-(4′-((2-(1,1-difluoroethyl)-6-ethylpyrimidin-4-yl)amino)-5-fluoro-6-(pyrrolidin-1-ylmethyl)-[2,3′-bipyridin]-6′-yl)acetamide;
N-(5-(6-(((1S,4S)-2-oxa-5-azabicyclo[2.2.1]heptan-5-yl)methyl)pyrimidin-4-yl)-4-((2-(1,1-difluoroethyl)-6-ethylpyrimidin-4-yl)amino)pyridin-2-yl)acetamide;
N-(6-(((1S,4S)-2-oxa-5-azabicyclo[2.2.1]heptan-5-yl)methyl)-4′-((2-(1,1-difluoroethyl)-6-ethylpyrimidin-4-yl)amino)-5-fluoro-[2,3′-bipyridin]-6′-yl)acetamide;
N-(5-(2-(((1R,4R)-2-oxa-5-azabicyclo[2.2.1]heptan-5-yl)methyl)pyrimidin-4-yl)-4-((2-(1,1-difluoroethyl)-6-ethylpyrimidin-4-yl)amino)pyridin-2-yl)acetamide;
N-(6-(((1R,4R)-2-oxa-5-azabicyclo[2.2.1]heptan-5-yl)methyl)-4′-((2-(1,1-difluoroethyl)-6-ethylpyrimidin-4-yl)amino)-5-fluoro-[2,3′-bipyridin]-6′-yl)acetamide;
N-(4-((2-(1,1-difluoroethyl)-6-ethylpyrimidin-4-yl)amino)-5-(2-(morpholinomethyl)pyrimidin-4-yl)pyridin-2-yl)acetamide;
N-(4′-((2-(1,1-difluoroethyl)-6-ethylpyrimidin-4-yl)amino)-5-fluoro-6-(morpholinomethyl)-[2,3′-bipyridin]-6′-yl)acetamide;
N-(4′-((2-(1,1-difluoroethyl)-6-ethylpyrimidin-4-yl)amino)-6-((dimethylamino)methyl)-5-fluoro-[2,3′-bipyridin]-6′-yl)acetamide;
N-(5-(((1R,5S)-6-oxa-3-azabicyclo[3.1.1]heptan-3-yl)methyl)-4′-((2-(1,1-difluoroethyl)-6-ethylpyrimidin-4-yl)amino)-[2,3′-bipyridin]-6′-yl)acetamide;
N-(5-(2-(((1R,5S)-6-oxa-3-azabicyclo[3.1.1]heptan-3-yl)methyl)pyrimidin-4-yl)-4-((2-(1,1-difluoroethyl)-6-ethylpyrimidin-4-yl)amino)pyridin-2-yl)acetamide;
N-(5-(6-(((1R,5S)-6-oxa-3-azabicyclo[3.1.1]heptan-3-yl)methyl)pyrimidin-4-yl)-4-((2-(1,1-difluoroethyl)-6-ethylpyrimidin-4-yl)amino)pyridin-2-yl)acetamide;
N-(6-(((1R,5S)-6-oxa-3-azabicyclo[3.1.1]heptan-3-yl)methyl)-4′-((2-(1,1-difluoroethyl)-6-ethylpyrimidin-4-yl)amino)-5-fluoro-[2,3′-bipyridin]-6′-yl)acetamide;
N-(4′-((2-(1,1-difluoroethyl)-6-methylpyrimidin-4-yl)amino)-5-(dimethylphosphoryl)-[2,3′-bipyridin]-6′-yl)acetamide;
N-(4-((2-(1,1-difluoroethyl)-6-methylpyrimidin-4-yl)amino)-5-(6-(dimethylphosphoryl)pyridazin-3-yl)pyridin-2-yl)acetamide;
N-(4-((2-(1,1-difluoroethyl)-6-ethylpyrimidin-4-yl)amino)-5-(2-methyl-2H-1,2,3-triazol-4-yl)pyridin-2-yl)acetamide;
1-(4-((2-(1,1-difluoroethyl)-6-ethylpyrimidin-4-yl)amino)-5-(pyrazin-2-yl)pyridin-2-yl)-3-methylurea;
1-(4-((2-(1,1-difluoroethyl)-6-ethylpyrimidin-4-yl)amino)-5-(pyrazin-2-yl)pyridin-2-yl)urea;
N-(4-((2-(1,1-difluoroethyl)-6-methylpyrimidin-4-yl)amino)-5-(5-(difluoromethyl)pyrazin-2-yl)pyridin-2-yl)acetamide; and
N-(5-(2-(cyanomethyl)thiazol-4-yl)-4-((2-(1,1-difluoroethyl)pyrimidin-4-yl)amino)pyridin-2-yl)acetamide;
   or a pharmaceutically acceptable salt thereof.

In a fifty-second embodiment, the present disclosure provides a pharmaceutical composition comprising a compound of the present disclosure (e.g., according to the first aspect or any one of the preceding embodiments), or a pharmaceutically acceptable salt thereof.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the invention include those wherein the solvent of crystallization may be isotopically substituted, e.g. D2O, d6-acetone, d6-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or IPLC chromatography columns, such as CHIRALPAKR™ and CHIRALCELR™ available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Methods of Use

The compounds disclosed herein have TYK2 activity. As used herein, “TYK2 inhibitory activity” refers to the ability of a compound or composition to induce a detectable decrease in TYK2 activity in vivo or in vitro (e.g., at least 10% decrease in TYK2 activity as measured by a given assay such as the bioassay described in the examples and known in the art).

In certain embodiments, the present disclosure provides a method of treating a disease or disorder responsive to inhibition of TYK2 activity (referred herein as “TYK2 mediated disease or disorder” or “disease or disorder mediated by TYK2”) in a subject in need of the treatment. The method comprises administering to the subject a compound described herein (e.g., a compound of Formula (I) according to any one of embodiments onto forty-two) or a pharmaceutically acceptable salt thereof or a pharmaceutical composition thereof.

In certain embodiments, the present disclosure provides the use of a compound described herein (e.g., a compound of Formula (I) according to any one of embodiments onto forty-two) or a pharmaceutically acceptable salt thereof, or a pharmaceutical composition comprising a compound described herein or a pharmaceutically acceptable salt thereof for the manufacture of a medicament for the treatment of a TYK2 mediated disorder or disease in a subject in need of the treatment.

In certain embodiments, the present disclosure provides a compound described herein (e.g., a compound of Formula (I) according to any one of embodiments onto forty-two) or a pharmaceutically acceptable salt thereof or a pharmaceutical composition comprising a compound described herein or a pharmaceutically acceptable salt thereof for use in the treatment of a TYK2 mediated disorder or disease in a subject in need of the treatment.

In some embodiments, the disease or disorder responsive to inhibition of TYK2 activity is inflammation, autoimmune disease, neuroinflammation, arthritis, rheumatoid arthritis, spondyloarthropathies, systemic lupus erythematosus, cutaneous lupus erythematosus, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, systemic sclerosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis or sunburn.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 g and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein, the term “cancer” has the meaning normally accepted in the art. The term can broadly refer to abnormal cell growth.

As used herein, the term “autoimmune disease” has the meaning normally accepted the art. The term can broadly refer to a disease where the host's immune system targets or attacks normal or healthy tissue of the host.

As used herein, the term “myelination” has the meaning normally accepted in the art. The term can broadly mean the process by which myelin is produced.

As used herein, the term “myelin-related disease or disorder”, “demyelinating disorder”, or “demyelation disorder” has the meaning normally accepted in the art. These terms can broadly refer to diseases or disorders which involve damage to myelin.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. In some embodiments, an optionally substituted group can be substituted with one or more substituents, each of which can the same or different. In some embodiments, the “one or more” substituents can be 1, 2, 3, 4, 5, 6, etc. substituents, each of which can the same or different. In some embodiment, the “one or more” substituents can be 1 to 6, 1 to 4, 1 to 3 or 1 to 2 substituents, each of which can the same or different. For the bicyclic heteroaryls and bicyclic, fused, spiro, or bridged heterocyclyls or carbocyclyls, the optional substituent can be on any or all of the rings. For example, when the bicyclic heteroaryl is depicted below, the substituent R^{3c} can be on either the 6-membered ring or the 5-membered ring.

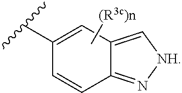

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄ alkyl group as defined herein, wherein at least of the hydrogen atoms is replaced by an C₁₋₄ alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono-or bi-cyclic. Monocyclic heteroaryl includes, for example, pyrazolyl, imidazolyl, oxazolyl, pyridinyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indoyl.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 6 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form. In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

According to a first process, compounds of Formula (I), may be prepared from compounds of Formulae (II′) and (III′), as shown in Scheme 1

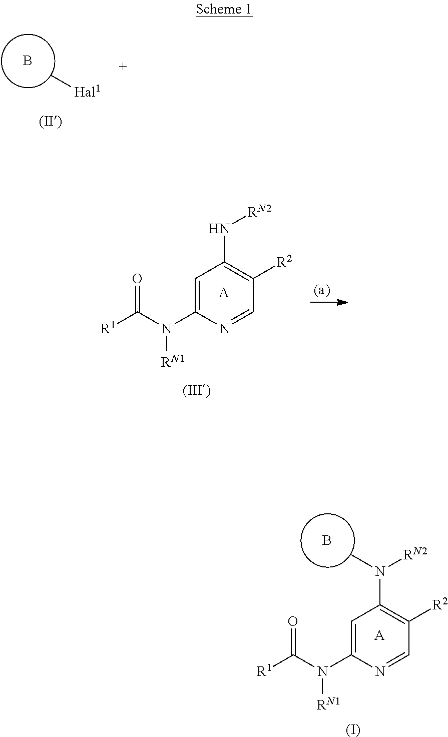

Hal¹ is halogen, preferably Br or Cl.

The compound of Formula (I) may be prepared from the compounds of Formulae (II′) and (III′) according to process step (a) a Buchwald-Hartwig cross coupling reaction. Typical conditions comprise, reaction of the compound of Formula (II′) with the amine of Formula (III′) in the presence of a suitable inorganic base, a suitable palladium catalyst in the presence of suitable phosphine ligands, in a suitable solvent at elevated temperature. Preferred conditions comprise, reaction of the compounds of Formulae (II′) and (III′) in the presence of Brettphos Pd G3, MolDalPhos Pd G3, Josiphos Pd G3, [XantPhos Pd(allyl)]Cl, or Xantphos or BINAP in combination with Pd(OAc)₂ or Pd₂(dba)₃, or Xantphos in combination with Xantphos Pd G3, in the presence of a suitable base such as NaOtBu, Cs₂CO₃, K₂CO₃ or K₃PO₄ in a suitable solvent such as dioxane, DMA, DMF or toluene at between 80° C. and 120° C.

According to a second process, compounds of Formula (I), may be prepared from compounds of Formulae (IV′) and (V′), as shown in Scheme 2

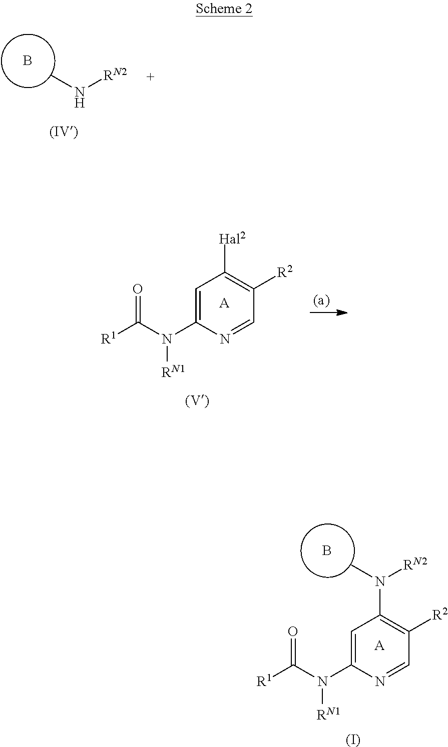

Hal² is halogen, preferably Br, Cl or I

The compound of Formula (I) may be prepared from the compounds of Formulae (IV′) and (V′), according to process step (a), a Buchwald reaction, as previously described in Scheme 1.

According to a third process, the compound of Formula (I) may be prepared from the compounds of Formulae (II′), (VI′), (VII′), (VIII′), (IX′), and (XV′) as shown in Scheme 3.

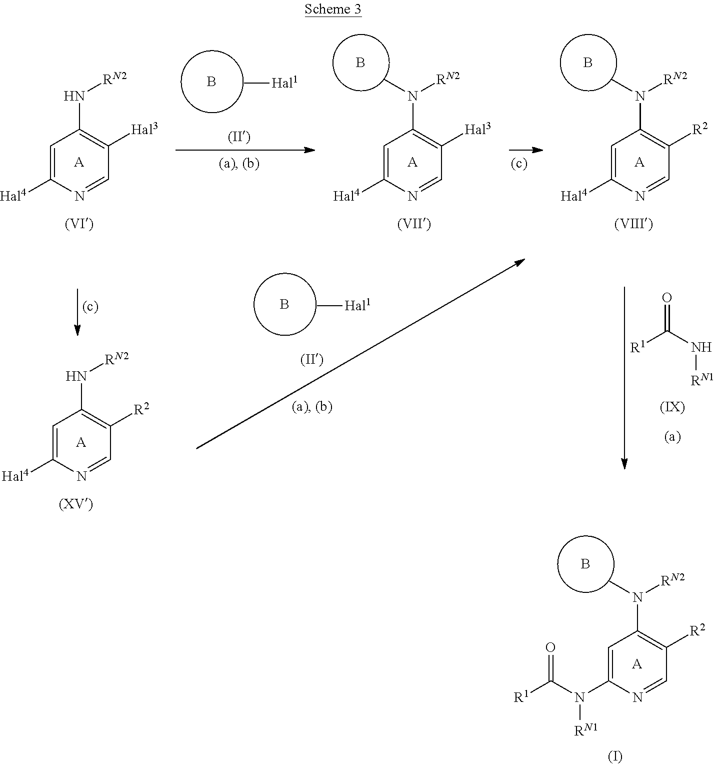

Hal³ is a halogen, preferably Br or I
Hal⁴ is a halogen, preferably Cl

The compound of Formula (VII′) may be prepared from the compounds of Formulae (VI′) and (II′) according to process step (b) an alkylation reaction, in the presence of a suitable inorganic base and a suitable aprotic polar solvent at between rt and elevated temperature. Preferred conditions, comprise reaction of the compound of Formula (VI′) with the compound of Formula (II′) in the presence of Cs₂CO₃ or K₂CO₃ in DMF or THF at between rt and 35° C.

Alternatively, the compound of Formula (VII′) may be prepared from the compounds of Formulae (VI′) and Formula (II′), according to process step (a), a Buchwald reaction, as previously described in Scheme 1.

Wherein, R² is a 4- to 11-membered monocyclic or bicyclic heterocyclic ring, C₁. 6alkyl or C₃₋₇cycloalkyl, the compound of Formula (VIII′) may be prepared from the compound of Formula (VII′) and R²BPin or R²B(OH)₂ according to process step (c) a palladium catalysed, cross-coupling reaction, such as a Suzuki reaction. Typical cross-coupling reaction conditions comprise a palladium catalyst containing suitable phosphine ligands, in the presence of an inorganic base, in a suitable aqueous solvent at between rt and the reflux temperature of the reaction. Preferred conditions comprise, reaction of the compound of Formula (VII′) and R²BPin, in the presence of SPhos-Pd-G3, Pd(PCy₃)₂, Pd(PPh₃)₄, XPhos Pd(crotyl)Cl, Pd(dppf)Cl₂, or Pd(OAc)₂ and P(Cy)₃ and a suitable base such as K₃PO₄, Na₂CO₃, or K₂CO₃ in a suitable solvent such as aqueous dioxane, DME, EtOH, or 2-MeTHF at between 70° C. and 100° C.

Wherein, R² is —OR^{2A} or —N(R^{2b})₂, the compound of Formula (VIII′) may be prepared from the compound of Formula (VII′) and HOR^{2A} or HN(R^{2b})₂, according to process steps (a) or (b) as previously described in Schemes 1 and 2.

Wherein, R² is a 4- to 11-membered monocyclic or bicyclic heterocyclic ring, C₁₋₆alkyl or C₃₋₇cycloalkyl, the compound of Formula (XV′) may be prepared from the compound of Formula (VI′) and R²BPin or R²B(OH)₂ according to process step (c) a palladium catalysed, cross-coupling reaction as described in Scheme 3. Wherein, R² is —OR^{2A} or —N(R^{2b})₂, the compound of Formula (XV′) may be prepared from the compound of Formula (VI′) and HOR^{2A} or HN(R^{2b})₂, according to process steps (a) or (b) as previously described in Schemes 1 and 2.

The compound of Formula (VIII′) may be prepared from the compounds of Formulae (XV′) and (II′) according to process steps (a) or (b), as previously described above.

The compound of Formula (I) may be prepared from the compound of Formula (VIII′) and R¹C(O)NR^{N1} according to process step (a), a Buchwald reaction as previously described in Scheme 1.

According to a fourth process, the compounds of Formula (III′) and (V′) may be prepared from the compounds of Formulae (IX′), (X′), (XI′), (XII′), (XIII′) and (XIV′) as shown in Scheme 4.

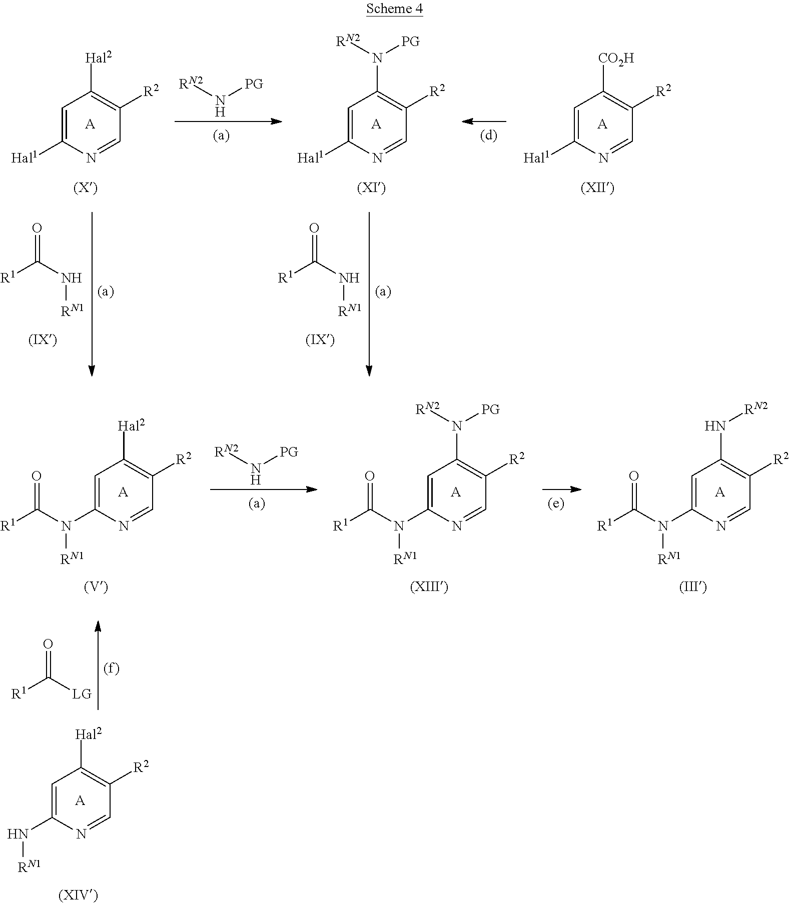

PG is a suitable N protecting group, typically a carbamate or substituted benzyl and preferably Boc or PMB.

LG is a suitable leaving group, such as a Cl or —OC(O)R¹.

The compound of Formula (XI′) may be prepared from the compound of Formula (X′) and R^{N2}NHPG according to process step (a), a Buchwald reaction, as previously described in Scheme 1.

Alternatively, the compound of Formula (XI′) may be prepared from the carboxylic acid of Formula (XII′) according to process step (d) a modified Curtius rearrangement. Preferred conditions comprise reaction of the carboxylic acid of Formula (XII′) with DPPA in t-BuOH in the presence of a suitable organic base, such as TEA at about 90° C.

The compound of Formula (V′) may be prepared from the compounds of Formulae (X′) and (IX′) according to process step (a), a Buchwald reaction, as previously described in Scheme 1.

Alternatively, the compound of Formula (V′) may be prepared from the amine of Formula (XIV′) and R¹C(O)LG according to process step (f) an amide bond formation.

Preferred conditions comprise reaction of the amine of Formula (XIV′) with R¹C(O)LG in a suitable solvent such as pyridine, optionally in the presence of an organic base such as DIPEA at about rt.

The compound of Formula (XIII′) may be prepared from the compounds of Formula (XI′) and (IX′) according to process step (a), a Buchwald reaction, as previously described in Scheme 1.

Alternatively, the compound of Formula (XIII′) may be prepared from the compound of Formula (V′) and R^{N2}NHPG according to process step (a), a Buchwald reaction, as previously described in Scheme 1.

The compound of Formula (III′) may be prepared from the compound of Formula (XIII′) according to process step (e) a de-protection reaction. Typical conditions comprise reaction of the compound of Formula (XIII′), wherein PG is Boc, with a suitable acid such as TFA or HCl in DCM or HFIP at rt or 2,6-lutidine and TMSOTf in DCM. Alternatively, wherein PG is PMB, typical conditions comprise ceric ammonium nitrate in aqueous MeCN at rt.

According to a fifth process, the compounds of Formula (I), wherein R² is a 4-11 membered monocyclic or bicyclic heterocycle, may be prepared from the compounds of Formulae (II′), (XX′), (XIX′), (XVIII′), (XVII′), (XVI′) and (XXVI′) as shown in Scheme 5.

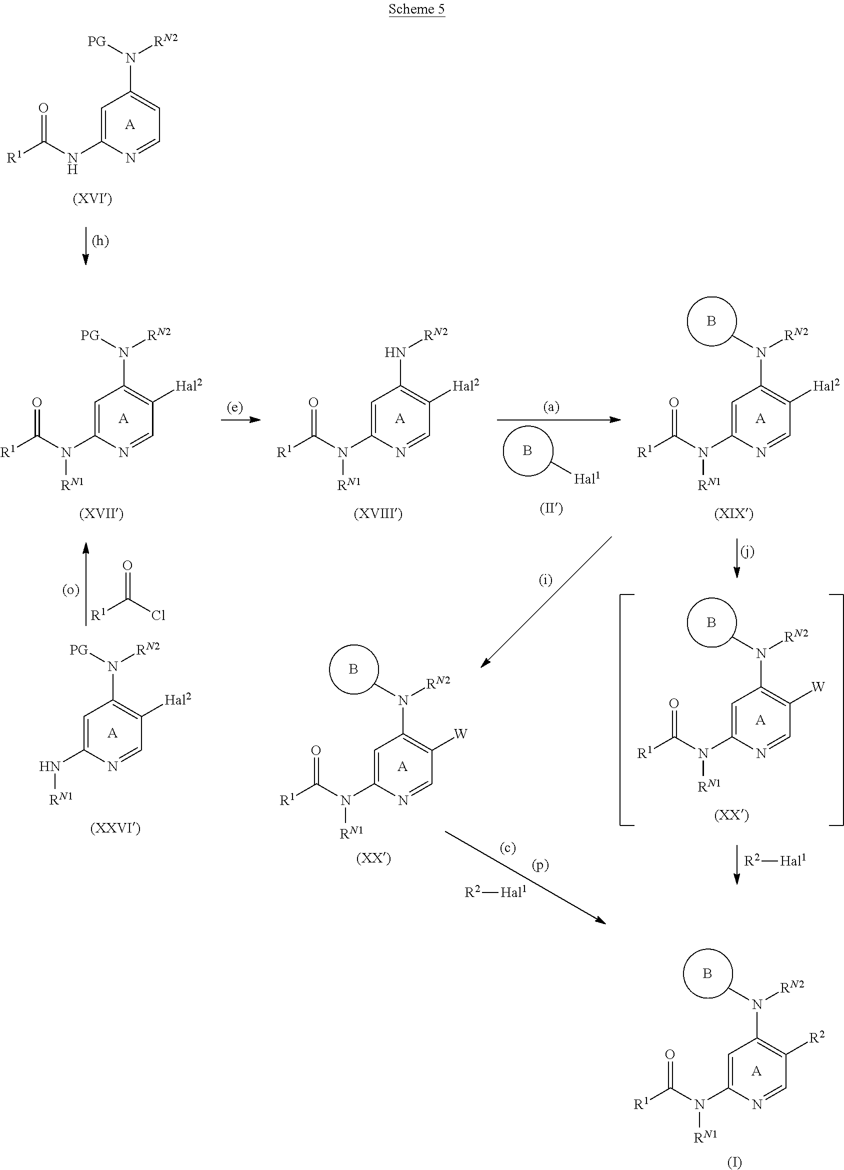

W is a boronic ester or boronic acid

The compound of Formula (XVII′) may be prepared from the compound of Formula (XVI′), by process step (h), a halogenation reaction, typically a bromination (h). Preferred conditions comprise reaction of the compound of Formula (XVI′) with NBS in acetonitrile at 70° C.

Alternatively, the compound of Formula (XVII′) may be prepared from the compound of Formula (XXVI′), by process step (o), an amide formation. Preferred conditions comprise reaction of the amine of Formula (XXVI′) with R¹COCl in the presence of an organic base, such as DIPEA in THF or dioxan at about rt.

The compound of Formula (XVIII′) may be prepared from the compound of Formula (XVII′), using process step (e), as previously described in Scheme 3.

The compound of Formula (XIX′) may be prepared from the compounds of Formulae (XVIII′) and (II′) using process step (a), as previously described in Scheme 1.

The compound of Formula (XX′) may be prepared from the compound of Formula (XIX′), according to step (i), a boronate ester formation achieved by treatment with a suitable boronate such as (BPin)₂, in the presence of a suitable inorganic base, such as K₂CO₃ or KOAc and a suitable catalyst, such as, BINAP, Brettphos, Xantphos, XPhos-Pd-G3, Pd(dppf)Cl₂, Pd₂(dba)₃ with a suitable phosphine ligand such as bis(1-adamantyl)butylphosphine, Pd(PPh₃)Cl₂ or Pd(PPh₃)₄. in a suitable non-polar solvent at between rt and elevated temperature.

The compound of Formula (I) may be prepared from the compound of Formula (XX′) and R²Hal¹ according to process step (c), a Suzuki-type boronic acid cross coupling reaction described above in Scheme 3.

Alternatively, the compound of Formula (I) may be prepared from the compound of Formula (XX′) and R²Hal¹ according to process step (p), a copper catalysed Suzuki cross coupling reaction. Preferred conditions comprise reaction of the compounds of Formula (XX′) and R²Hal¹ in the presence of a copper catalyst such as Cu(OAc)₂ and DMAP in MeCN at about 40° C.

Alternatively, the compound of Formula (I) may be obtained from the compound of Formula (XIX′) via step (j), by an in situ process without isolation of the compound of Formula (XX′). Preferred conditions comprise reaction of the compounds of Formulae (XIX′) and R²—Hal¹ in the presence of bis(pinacolato)diboron (W₂), Pd(OAc)₂, in the presence of a suitable phosphane ligand such as bis(1-adamantyl)-butyl-phosphane, a suitable base such as CsF in a suitable solvent such as toluene, MeOH, water at between 80° C. and 120° C.

According to a sixth process, compounds of Formula (XIII′), may be obtained from the compounds of Formula (XVII′) and (XXI′) as shown in Scheme 6.

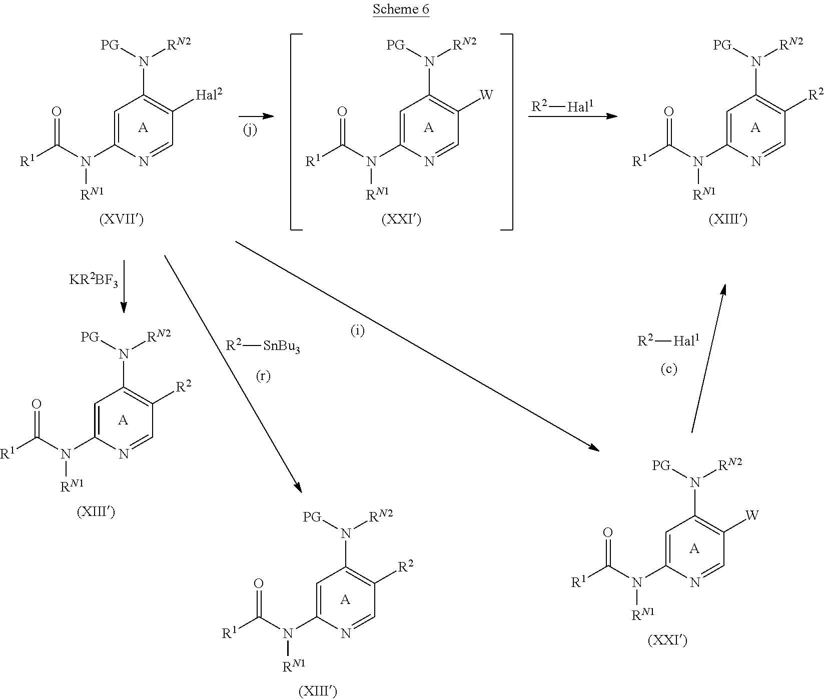

Wherein R² is a 4-11 membered monocyclic or bicyclic heterocycle, the compound of Formula (XIII′) may be prepared from the compound of Formula (XVII′) and R²Hal¹ according to process step (j), as previously described in Scheme 5.

Alternatively, the compound of Formula (XXI′) may be prepared according to process step (i) as previously described in Scheme 5. The compound of Formula (XIII′) may be prepared from the compound of Formula (XXI′) and R²Hal¹ according to process step (c) as previously described in Scheme 3.

Alternatively, the compound of Formula (XIII′) may be prepared from the compound of Formula (XVII′) and R²—SnBu₃ according to process step (r), a Stille type cross-coupling reaction. Preferred conditions comprise, reaction of the compound of Formula (XVII′) with R²SnBu₃, in the presence of a suitable catalyst such as PdCl₂(PPh₃)₂ in DMF, at about 100° C.

Wherein R² is alkyl or cycloalkyl, the compound of Formula (XIII′) may be obtained from the compound of Formula (XVII′) and KR²BF₃, according to process step (q), a palladium catalysed Suzuki coupling. Preferred conditions comprise reaction of the compound of Formula (XVII′) and KR²BF₃ in the presence of catalyst, cataCXium A and KOAc, a suitable base such as Cs₂CO₃ in aq toluene at about 90° C.

According to a seventh process, compounds of Formula (XIII′), wherein R² is C₂-C₆ alkyl, may be obtained from the compounds of Formula (XVII′), (XXII′) and (XXIII′) as shown in Scheme 7.

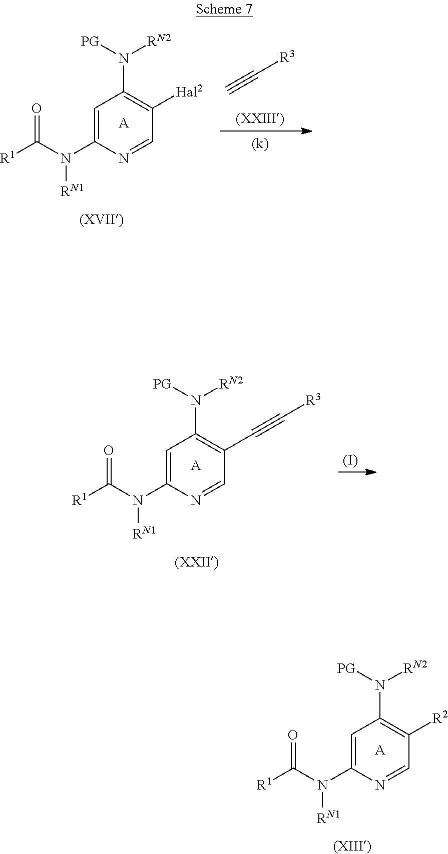

R³—C≡C is an unsaturated pre-cursor to R²

The compound of Formula (XXII′) may be prepared from compounds of Formulae (XVII′) and (XXIII′) by process step (k), a Sonogashira type cross-coupling reaction. Typical conditions comprise reaction of the compound of Formula (XVII′) with an alkyne of Formula (XXIII′) in the presence of, a suitable palladium catalyst in the presence of suitable phosphine ligands, Cu salt and base, in a suitable solvent at elevated temperature. Preferred conditions comprise, reaction of the compounds of Formulae (XVII′) and (XXIII′) in the presence of Pd(PPh₃)₂Cl₂·DCM, CuI and a suitable base such as TEA in a solvent such as DMF at between 80° C. and 120° C.

The compound of Formula (XIII′) may be prepared from the compound of Formula (XXII′) by step (1), a hydrogenation reaction. Typical conditions comprise, reaction of the compound of Formula (XXII′) in the presence of a Pd catalyst and H₂ in THF at rt.

According to an eighth process, compounds of Formula (I), wherein R² is a 4-11 membered monocyclic or bicyclic heterocycle, may be obtained from the compounds of Formula (XXIV′), (XXV′) and (XIII′) as shown in Scheme 8.

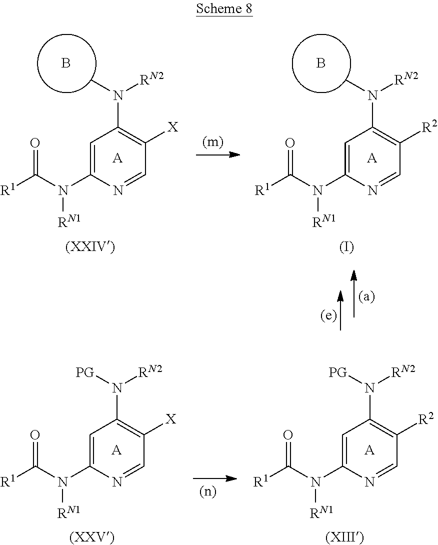

X is a carbonyl group, preferably a carboxylic ester, or a nitrile.

Compounds of Formula (I) may be prepared from compounds of Formulae (XXIV′) or (XXV′) using standard chemical transformations (m) known to those skilled in the art to convert carboxylic esters or other carboxyl derivatives into heterocycles. E.g methyl ester (XXIV′), X=CO₂Me, may be hydrolysed with LiOH to carboxylic acid (XXIV′), X=CO₂H, and subsequently treated with N′-hydroxyacetimidamide (acetamide oxime) to obtain 3-methyl-1,2,4-oxadiazoles.

Compounds of Formula (XIII′) may be obtained from compounds of Formula (XXV′) using standard chemical transformations (n) known to those skilled in the art to convert nitriles or other carboxyl derivatives into heterocycles. E.g. nitrile (XXV′), X=CN, may be reacted with hydroxylamine and subsequently with N′—1,1-dimethoxy-N,N-dimethylethan-1-amine to obtain 5-methyl-1,2,4-oxadiazoles.

According to a ninth process, the compounds of Formula (I), wherein R^{N2} is H, may be obtained from the compounds of Formula (XXVII′) and (XXVIII′) as shown in Scheme 9.

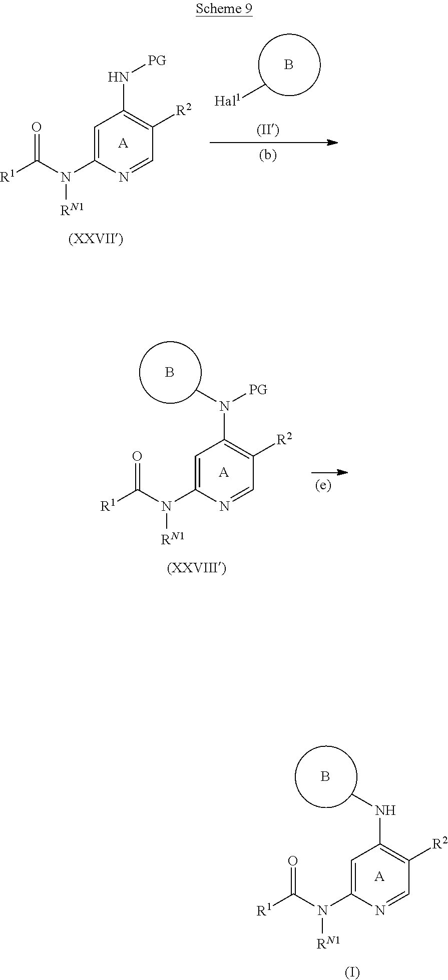

The compound of Formula (XXVIII′) may be obtained from the compounds of Formula (XXVII′) and (II′), by process step (b), an alkylation reaction, as previously described in Scheme 3.

The compound of Formula (I), wherein R^{N2} is H, may be obtained by the deprotection of the compound of Formula (XXVIII′), according to process step (e), a deprotection reaction, as previously described in Scheme 4.

According to a tenth process, wherein R² is a heteroalkyl or alkyl, the compounds of Formula (I) may be obtained from the compound of Formula (XIX′) as shown in Scheme 10.

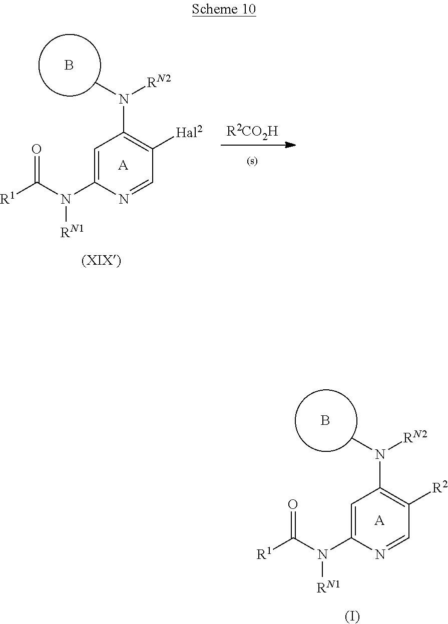

The compound of Formula (I) may be obtained from the compound of Formula (XIX′) and R²CO₂H according to process step (s), a photo catalysed cross-coupling reaction.

Preferred conditions comprise reaction of the compound of Formula (XIX′) with R²CO₂H, in the presence of Ir[dF(CF₃)ppy]₂(dtbpy)PF₆, NiBr₂(dtbbpy) and (2-tert-butyl-1,1,3,3-tetramethyl-guanidine in DMF at rt, under an LED light source.

The compounds of Formulae (II′), (IV′), (VI′), (IX′), (X′), (XII′), (XIV′), (XV′), (XVI′), (XVII′), (XXIII′), (XXIV′), (XXV′), (XXVI′), (XXVII′), R^{N2}NH-PG, R²CO₂H, and R¹C(O)LG are either commercially available or may be prepared by analogy to methods known in the literature, or the methods described in the Experimental section below.

Compounds of Formulae (I), (III′), (IV′), (V′), (VII′), (VIII′), (XI′), (XIII′), (XIV′) (XV′), (XIX′) (XXIV′) (XXVI′) and (XXVII′) may be converted to alternative compounds of Formulae (I), (III′), (IV′), (V′), (VII′), (VIII′) (XI′), (XIII′), (XIV′) (XV′), (XIX′) (XXIV′) (XXVI′) and (XXVII′) respectively, by standard chemical transformations, known to those skilled in the art. Examples of these transformations include, but are not limited to:
alkyation of a heteroatom, such as N or O, using an alkyl or aryl halide, a suitable inorganic or organic base.
reductive amination oa a N atom to provide a secondary or tertiary amine.
reaction of an aryl or heteroaryl halide with an alcohol in the presence of an inorganic base to provide an aryl or heteroaryl ether.
reaction of an alkyl halide with a primary or secondary amine or alcohol in the presence of a base to provide a secondary or tertiary amine or ether.
reaction of a primary alcohol with an alternative alcohol under Mitsunobu conditions to provide an ether
fluorination of aryl chloride with a fluorinating agent, such as TBAF, to provide an aryl fluoride
demethylation of an alkyl methoxy group to provide an alkyl hydroxy group
photocatalysed reaction of an aryl or heteroaryl halide with a (1,3-dioxoisoindolin-2-yl)alkyl, cycloalkyl or heterocycyl carboxylate to provide an alkyl, cycloalkyl or heterocyclyl substituted aryl or heterocycle
transition metal catalysed cross-coupling of an aromatic or heteroaryl halide with an
   appropriate boronic acid or boronate ester under Suzuki reaction conditions as described in step (c), (p) or (q),
   appropriate alkyl or heteroaryl zinc compound under Negishi type reaction conditions,
   appropriate alkyl or heteroaryl stannane under Stille type reaction conditions
   amine or alcohol under Buchwald type conditions, as described in process step (a)
to provide the corresponding substituted aromatic or heteroaromatic group.

It will be appreciated by those skilled in the art that it may be necessary to utilise a suitable protecting group strategy for the preparation of compounds of Formula (I). Typical protecting groups may comprise, a carbamate group, preferably Boc for the protection of amines, a benzyl group for the protection of a phenolic OH or a TBS group for the protection of an alkyl OH.

It will be further appreciated that it may be necessary or desirable to carry out the transformations in a different order from that described in the schemes, or to modify one or more of the transformations, to provide the desired compound of the invention.

### EXEMPLIFICATION

### Abbreviations

AcOH=acetic acid;
Ac₂O=acetic anhydride;
Aq.=aqueous;
Bn=benzyl;
BnOH=benzyl alcohol;
Boc=tert-butoxy carbonyl;
Boc₂O=di-tert-butyl dicarbonate;
br=broad;
Brettphos Pd G3=[(2-di-cyclohexylphosphino-3,6-dimethoxy-2′,4′,6′-triisopropyl-1,1′-biphenyl)-2-(2′-amino-1,1′-biphenyl)]palladium(II) methanesulfonate;
t-BuOH=tert-butanol;
t-BuONa=sodium tert-butoxide;
n-BuLi=n-butyl lithium;
° C.=degrees Celsius;
CDCl₃=deutero-chloroform;
Cs₂CO₃=cesium carbonate;
CuI=copper (I) iodide;
δ=chemical shift;
d=doublet;
DAST=(diethylamino)sulfur trifluoride;
dd=double doublet;
DCE=1,2-dichloroethane;
DCM=dichloromethane;
DIPEA=N-ethyldiisopropylamine or N,N-diisopropylethylamine;
DMAP=4-(Dimethylamino)pyridine;
DME=1,2-dimethoxyethane;
DMF=N,N-dimethylformamide;
DMSO=Dimethylsulfoxide;
DMSO-d₆=hexadeuterodimethyl sulfoxide;
DPPA=diphenyl phosphoryl azide;
Et=ethyl;
EtOH=ethanol;
EtOAc=ethyl acetate;
Eq.=equivalent;
g=gram;
HCl=hydrochloric acid;
HCO₂H=formic acid;
¹H NMR=proton nuclear magnetic resonance;
H₂O=water;
HPLC=high pressure liquid chromatography;
h=hour;
IPA=2-propanol;
Josiphos Pd G3={(R)-1-[(Sp)-2-(Dicyclohexylphosphino)ferrocenyl]ethyldi-tert-butylphosphine}[2-(2′-amino-1,1′-biphenyl)]palladium(II) methanesulfonate;
K₂CO₃=potassium carbonate;
KF=potassium fluoride;
KOH=potassium hydroxide;
KOtBu=potassium tert-butoxide;
K₃PO₄=potassium phosphate tribasic;
L=litre;
LCMS=liquid chromatography mass spectrometry;
LiAlH₄=lithium aluminium hydride;
m=multiplet;
M=molar;
m-CPBA=3-chloroperoxybenzoic acid;
Me=methyl;
MeCN=acetonitrile;
MeI=iodomethane;
MeOH=methanol;
MeOH-d₄=deutero-methanol;
mg=milligram;
MgSO₄=magnesium sulfate;
MHz=mega Hertz;
mins=minutes;
mL=millilitres;
mmol=millimole;
MorDalPhos Pd G3=mesyl(2-(di-1-adamantylphosphino)morpholinobenzene)[2-(2′-amino-1,1′-biphenyl)]palladium(II);
MS m/z=mass spectrum peak;
MTBE=tert-butyl methyl eher;
N₂=nitrogen;
NaBH₄=sodium borohydride;
NaBH₃CN=sodium cycanoborohydride;
Na₂CO₃=sodium carbonate;
NaH=sodium hydride;
NaHCO₃=sodium bicarbonate;
NaOH=sodium hydroxide;
NaOMe=sodium methoxide;
Na₂SO₃=sodium thiosulfate;
Na₂SO₄=sodium sulfate;
NBS=N-bromosuccinimide;
NH₂Boc=tert-butyl carbamate;
NH₃=ammonia;
NH₄Cl=ammonium chloride;
NH₄OH is ammonium hydroxide;
NMP=1-methyl-2-pyrrolidone;
PE=petroleum ether;
Pd(dppf)Cl₂=[1,1′-bis(diphenylphosphino)ferrocene]dichloropalladium(II);
Pd(OAc)₂=palladium (II) acetate;
Pd₂(dba)₃=tris(dibenzylideneacetone)dipalladium (0);
Pd(PPh₃)₂Cl₂=bis(triphenylphosphine)palladium(II) chloride;
POCl₃=phosphorus (V) oxychloride;
q=quartet;
rt=room temperature;
RT=retention time;
s=singlet;
sat.=saturated;
soln.=solution;
t=triplet;
TBSCl=tert-butyldimethylsilyl chloride;
TEA=triethylamine;
TFA=trifluoroacetic acid;
THF=tetrahydrofuran;
TLC=thin layer chromatography;
μL=micro litres;
μmol=micromole;
Xantphos=4,5-Bis(diphenylphosphino)-9,9-dimethylxanthene;
Xantphos Pd G3=[(4,5-bis(diphenylphosphino)-9,9-dimethylxanthene)-2-(2′-amino-1,1′-biphenyl)]palladium(II) methanesulfonate.

### General Methods

### Prep HPLC Conditions:

Method A: Column: YMC-Triart Prep C18 150*40 mm*7 um; Condition: water (NH₄HCO₃)-MeCN; Gradient Time (min): 15; 100% B Hold Time (min): 3; Flow Rate (mL/min): 50; Detection wavelength: 220 nm
Method B: Column: Welch Xtimate C18 150*25 mm*5 um; Condition: water (10 mM NH₄HCO₃)-MeCN; Gradient Time (min): 11; 100% B Hold Time (min): 2; Flow Rate (mL/min): 25; Detection wavelength: 220 nm)
Method C: Column: Boston Prime C18 150*30 mm*5 um; Condition: water (10 mM NH₄HCO₃)-MeCN; Gradient Time (min): 10; 100% B Hold Time (min): 2; Flow Rate (mL/min): 25; Detection wavelength: 220 nm)
Method D: Column: Boston Green ODS 150*30 mm*5 um, Condition: water (FA)-MeCN; Gradient Time (min): 10; 100% B Hold Time (min): 2; Flow Rate (mL/min): 25
Method E: Column: Boston Uni C18 40*150*5 um; Condition: water (TFA)-MeCN; Gradient Time (min): 10; 100% B Hold Time (min): 2; Flow Rate (mL/min): 60
Method F: Column: Shapsil-T C18 50*250 mm 8 um, Condition: water (0.1% FA)-MeCN, Gradient: Gradient time: 20 min, Flow rate 80 mL/min
Method G: Column: Boston Green ODS 150*30 mm*5 um; Condition:water (NH₄HCO₃)-MeCN; Gradient Time (min): 11; Flow Rate (mL/min): 25
Method H: Column: Boston Green ODS 150*30 mm*5 um; Condition: water (HCl)-MeCN; Gradient Time (min): 10; 100% B Hold Time (min): 2; Flow Rate (mL/min): 25
Method I: YMC-Actus Triart C18 150*30 mm*5 um, Condition: water (TFA)-MeCN; Gradient Time (min): 10.5; 100% B Hold Time (min): 1.5; Flow Rate (mL/min): 40
Method J: Waters Sunfire Prep C18, 5 m, 19 mm×100 mm column with mobile phase H₂O and MeCN (0.2% NH₄HCO₃ final v/v % modifier) with flow rate at 30 mL/min.
Method K: Column: Boston Prime C18 150*30 mm*5 um; Condition: water (NH₃·H₂O+NH₄HCO₃)-MeCN; Gradient Time (min): 10; 100% B Hold Time (min): 2; Flow Rate (ml/min): 25.
Method L: Column: Welch Xtimate C18 150*25 mm*5 um; Condition: water (ammonia hydroxide v/v)-MeCN; Gradient Time (min): 11; 100% B Hold Time (min): 2; Flow Rate (ml/min): 25;
Method M: Column: Boston Prime C18 150*30 mm*5 um; Condition: water (FA)-MeCN; Gradient Time (min): 12; 100% B Hold Time (min): 2; Flow Rate (ml/min): 25.
Method N: Column: Phenomenex C18 150*25 mm*10 um; mobile phase: water (NH₄HCO₃)-MeCN; Gradient Time (min): 10; 100% B Hold Time (min): 2; Flow Rate (ml/min): 25.
Method O: Column: Phenomenex C18 150*40 mm*5 um; Condition: water (NH₃·H₂O+NH₄HCO₃)-MeCN; Gradient Time (min): 10; 100% B Hold Time (min): 2; Flow Rate (ml/min): 25.
Method P: Column:Waters Xbridge BEH C18 100*30 mm*10 um; Condition: water (NH₄HCO₃)-MeCN; Gradient Time (min): 11; 100% B Hold Time (min): 3; Flow Rate (ml/min): 25.
Method Q: Column: Phenomenex Gemini-NX 150*30 mm*5 um; Condition: water (NH₃H₂O+NH₄HCO₃)-MeCN; Gradient Time (min): 15; 100% B Hold Time (min): 3; Flow Rate (ml/min): 25.
Method R: Column: Phenomenex Gemini-NX 150*30 mm*5 um: water (NH₄HCO₃)-MECN; Gradient Time (min): 11; Flow Rate (ml/min): 25.
Method S: Column: C18-1 150*30 mm*5 um; Condition: water (NH₄HCO₃)-MECN; Begin B: Gradient Time (min): 11; Flow Rate (ml/min): 25.
Method T: Column: Boston Green ODS 150×30 mm, 5 mm; MeCN/H₂O (TFA).
Method U: Column: Waters XSelect CSH C18, 5 m, 30 mm×100 mm column with mobile phase H₂O (A) and MeCN (B) (0.2% NH₄OH final v/v % modifier) with flow rate at 60 mL/min).
Method V: Column: Waters Sunfire Prep C18 OBD 5 μm 19×100 mm, Gradient: MeCN in water, modifier 0.1% TFA, Gradient time, 12 min. Flow rate: 30 ml/min.
Method W: Column: Waters Xbridge BEH C18 100*30 mm*10 um, Condition: water (HCl)-MeCN; Gradient Time (min): 11; Flow Rate (ml/min): 50).
Method X: Column: Waters Xbridge BEH C18 100*30 mm*10 um; Condition: water (FA)-MeCN; Gradient Time (min): 11; 100% B Hold Time (min): 3; Flow Rate (ml/min): 50.
Method Y: Column: Phenomenex luna C18 150*25 mm*10 um; Condition: water (FA)-MeCN; gradient over 10 min, flow rate 25 mL/min.
Method Z: Waters Xbridge BEH C18 100×30 mm, 10 mm; MeCN/H₂O (TFA))
Method AA: Column: Agela DuraShell C18 150*25 mm*5 um; Condition: water (NH₃·H₂O)-MECN; B %: gradient over 10 min.

### Preparation 1

### 2-bromo-5-ethoxypyridine 1-oxide

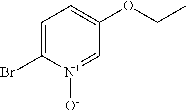

To a solution of 2-bromo-5-ethoxypyridine (27.0 g, 134 mmol) in DCE (300 mL) was added m-CPBA (46.1 g, 267 mmol) and the reaction was stirred at 80° C. for 2 h. The mixture was adjusted to pH=10 with ethane-1,2-diamine (16.1 g, 267 mmol), concentrated in vacuo, diluted with water (150 mL) and extracted with EtOAc (200 mL×3). The combined organic phase was washed with brine (300 mL), dried over anhydrous Na₂SO₄, filtered and concentrated. The crude was purified by chromatography on silica gel (EtOAc) to give 2-bromo-5-ethoxypyridine 1-oxide (23.0 g, 78.6% yield) as a white solid. LCMS m/z=218.0 [M+H]⁺.

### Preparation 2

### 2-bromo-5-ethoxy-4-nitropyridine 1-oxide

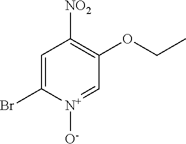

To a solution of 2-bromo-5-ethoxypyridine 1-oxide (Preparation 1, 11.0 g, 50.5 mmol) in H₂SO₄ (12.0 mL) was added HNO₃ (12.0 mL) dropwise and the reaction was stirred at 25° C. for 16 h. The mixture was poured into an ice/water mixture (200 mL). The solid was collected by filtration and washed with ice/water to give 2-bromo-5-ethoxy-4-nitropyridine 1-oxide (15.2 g, crude) as a light-yellow solid. ¹H NMR: (400 MHz, CDCl₃) δ ppm: 8.27 (s, 1H), 8.25 (s, 1H), 4.19 (q, J=6.8 Hz, 2H), 1.53 (t, J=6.8 Hz, 3H)

### Preparation 3

### 2-bromo-5-ethoxypyridin-4-amine

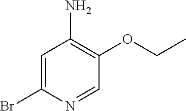

To a solution of 2-bromo-5-ethoxy-4-nitropyridine 1-oxide (Preparation 2, 15.0 g, 56.8 mmol) in AcOH (50.0 mL) was added iron (15.9 g, 284 mmol). The reaction was stirred at 25° C. for 30 min and the mixture was then diluted with water (500 mL) and extracted with EtOAc (400 mL×3). The combined organic phase was washed with brine (100 mL×3), dried over anhydrous Na₂SO₄, filtered and concentrated to give 2-bromo-5-ethoxypyridin-4-amine (5.64 g, 45.7% yield) as a brown solid. LCMS m/z=219.0 [M+H]⁺.

### Preparation 4

### tert-butyl (2-bromo-5-ethoxypyridin-4-yl)(tert-butoxycarbonyl)carbamate

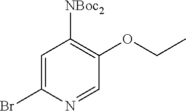

To a solution of 2-bromo-5-ethoxypyridin-4-amine (Preparation 3, 5.6 g, 25.9 mmol) and TEA (7.0 g, 69.2 mmol) in DCM (50 mL) was added DMAP (3.2 g, 25.9 mmol) and Boc₂O (22.6 g, 104 mmol) and the reaction was stirred at 25° C. for 2 h. The mixture was diluted with water (50 mL) and extracted with EtOAc (70 mL×3). The combined organic phase was washed with brine (50 mL×3), dried over anhydrous Na₂SO₄, filtered and concentrated. The crude was purified by chromatography on silica gel (PE/EtOAc 1/0 to 3/1) to give tert-butyl (2-bromo-5-ethoxypyridin-4-yl)(tert-butoxycarbonyl)carbamate (1.7 g, 15.5% yield) as a yellow solid. LCMS m/z=419.0 [M+H]⁺.

### Preparation 5

### tert-butyl (2-acetamido-5-ethoxypyridin-4-yl)(tert-butoxycarbonyl)carbamate

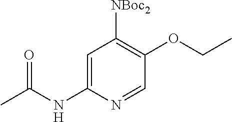

To a solution of tert-butyl (2-bromo-5-ethoxypyridin-4-yl)(tert-butoxycarbonyl)carbamate (Preparation 4, 1.7 g, 4.00 mmol) in dioxane (20 mL) was added acetamide (260 mg, 4.40 mmol), Cs₂CO₃ (2.6 g, 8.00 mmol) and BrettPhos Pd G3 (363 mg, 0.40 mmol) and the reaction was stirred at 100° C. for 1 h. The mixture was diluted with water (15 mL) and extracted with EtOAc (20 mL×3). The combined organic phase was washed with brine (60 mL), dried over anhydrous Na₂SO₄, filtered and concentrated. The crude product was purified by chromatography on silica gel (PE/EtOAc 1/0 to 2/1) to give tert-butyl (2-acetamido-5-ethoxypyridin-4-yl)(tert-butoxycarbonyl)carbamate (1.3 g, 82.1% yield) as a light-yellow solid. LCMS m/z=396.2 [M+H]⁺.

### Preparation 6

### 2-chloro-5-(difluoromethoxy)-4-iodopyridine

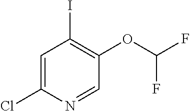 [SMILES: FC(F)Oc1cnc(Cl)cc1I]

To a solution of 6-chloro-4-iodopyridin-3-ol (1 g, 3.91 mmol) in DMF (20 mL) was added sodium 2-chloro-2,2-difluoroacetate (1.2 g, 7.83 mmol) and Cs₂CO₃ (2.6 g, 7.83 mmol) and the reaction was stirred at 100° C. for 4 h under N₂. The mixture was quenched with H₂O (20 mL) and extracted with EtOAc (20 mL×2). The combined organic phase was washed with brine (20 mL×2), dried over Na₂SO₄, filtered, and the filtrate was concentrated in vacuo. The residue was purified by chromatography on silica gel (PE/EtOAc=3/1) to give 2-chloro-5-(difluoromethoxy)-4-iodopyridine (1.2 g, 97% yield) as a yellow oil. ¹H NMR (400 MHz, CDCl₃) δ: ppm 8.17 (s, 1H), 7.84 (s, 1H), 6.76-6.40 (m, 1H).

### Preparation 7

### 2-chloro-5-cyclobutoxy-4-iodopyridine

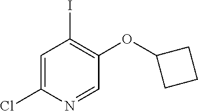 [SMILES: Clc1cc(I)c(OC2CCC2)cn1]

2-Chloro-5-cyclobutoxy-4-iodopyridine was obtained, 940 mg, 77.6% yield, from 6-chloro-4-iodopyridin-3-ol and bromocyclobutane following the procedure described in Preparation 6. LCMS m/z=309.8 [M+H]⁺

### Preparation 8

### 2-chloro-4-iodo-5-isopropoxypyridine

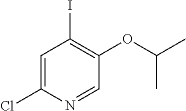

2-Chloro-4-iodo-5-isopropoxypyridine was obtained as a yellow liquid, 2.2 g, 94.4% yield, from 6-chloro-4-iodopyridin-3-ol and 2-iodopropane, following the procedure described in Preparation 6. 1H NMR (500 MHz, CDCl₃) δ ppm: 7.82 (s, 1H), 7.73 (s, 1H), 4.66-4.59 (m, 1H), 1.45-1.38 (m, 6H).

### Preparation 9

### tert-butyl (2-chloro-5-(difluoromethoxy)pyridin-4-yl)carbamate

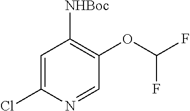 [SMILES: CC(C)(C)OC(=O)Nc1cc(Cl)ncc1OC(F)F]

To a solution of 2-chloro-5-(difluoromethoxy)-4-iodopyridine (Preparation 6, 1.1 g, 3.44 mmol) in dioxane (20 mL) was added NH₂Boc (382.6 mg, 3.27 mmol), Cs₂CO₃ (2.24 g, 6.88 mmol), Xantphos (596.7 mg, 1.03 mmol) and Pd₂(dba)₃ (314.8 mg, 0.344 mmol) and the reaction was stirred at 100° C. for 16 h under N₂. The cooled mixture was concentrated and the residue was purified by chromatography on silica gel (PE/EtOAc=3/1) to give tert-butyl (2-chloro-5-(difluoromethoxy)pyridin-4-yl)carbamate (940 mg, 93.0% yield) as a yellow oil. ¹H NMR: (400 MHz, CDCl₃) δ: ppm 8.24 (s, 1H), 8.12 (s, 1H), 7.07 (s, 1H), 6.75-6.36 (m, 1H), 1.54 (s, 9H).

### Preparation 10

### tert-butyl (2-chloro-5-cyclobutoxypyridin-4-yl)carbamate

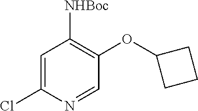 [SMILES: CC(C)(C)OC(=O)Nc1cc(Cl)ncc1OC1CCC1]

To a solution of 2-chloro-5-cyclobutoxy-4-iodopyridine (Preparation 7, 930 mg, 3.0 mmol) and NH₂Boc (387.2 mg, 3.31 mmol) in dioxane (15 mL) was added Cs₂CO₃ (2.94 g, 9.01 mmol), Pd₂(dba)₃ (275.13 mg, 0.3 mmol) and Xantphos (347.70 mg, 0.6 mmol) and the reaction was stirred at 100° C. for 1.5 h under N₂. The cooled mixture was diluted with water (50 mL) and extracted with EtOAc (70 mL×3). The combined organic phase was washed with brine (50 mL×3), dried over anhydrous Na₂SO₄ and concentrated to give tert-butyl (2-chloro-5-cyclobutoxypyridin-4-yl)carbamate (1.4 g, crude) as a white solid. LCMS m/z=299.1 [M+H]⁺

### Preparation 11

### tert-butyl (2-chloro-5-isopropoxypyridin-4-yl)carbamate

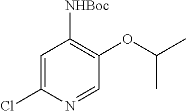 [SMILES: CC(C)Oc1cnc(Cl)cc1NC(=O)OC(C)(C)C]

The title compound was obtained as a yellow solid, 1.03 g, 49% yield from 2-chloro-4-iodo-5-isopropoxypyridine (Preparation 8) and NH₂Boc following the procedure described in Preparation 9. ¹H NMR (400 MHz, CDCl₃) δ ppm: 8.09 (s, 1H), 7.84 (s, 1H), 7.18 (s, 1H), 4.66-4.59 (m, 1H), 1.54 (s, 9H), 1.40 (d, J=6.4 Hz, 6H).

### Preparation 12

### 2-bromo-5-(2-methoxyethoxy)isonicotinic acid

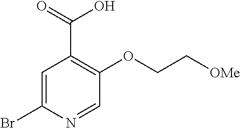 [SMILES: COCCOc1cnc(Br)cc1C(=O)O]

t-BuONa (21.8 g, 227 mmol) was added to a solution of 2-bromo-5-fluoroisonicotinic acid (10.0 g, 45.5 mmol) and 2-methoxyethan-1-ol (100 mL) and the reaction was stirred at 70° C. for 1 h. The mixture was concentrated and the residue was dissolved in H₂O (10 mL) and adjusted to pH ˜4 with 2N HCl. The precipitate was isolated by filtration, dissolved in EtOAc (25 mL) and washed with brine (25 mL). The organic layer was dried over Na₂SO₄, filtered and concentrated to give 2-bromo-5-(2-methoxyethoxy)isonicotinic acid (10.0 g, 79.7% yield) as a yellow solid. ¹H NMR: (400 MHz, DMSO-d₆) δ ppm: 13.63 (br s, 1H), 8.35 (s, 1H), 7.69 (s, 1H), 4.29-4.27 (m, 2H), 3.67-3.64 (m, 2H), 3.30 (s, 3H).

### Preparation 13

### 2-bromo-5-ethoxyisonicotinic acid

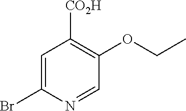

To a solution of 2-bromo-5-fluoroisonicotinic acid (110 g, 500 mmol) in EtOH (1500 mL) was added t-BuONa (120.13 g, 1.25 mol) and the reaction was stirred at 90° C. for 12 h under N₂. The mixture was concentrated under reduced pressure and the residue was diluted with water (500 mL). The water phase was adjusted to pH 4 with 12M HCl, the resulting mixture was filtered and the filter cake was washed with water (200 mL×2) and concentrated under reduced pressure to give 2-bromo-5-ethoxyisonicotinic acid (115 g, 93.5% yield) as yellow solid. ¹H NMR (400 MHz, DMSO-d₆) δ ppm 8.32 (s, 1H), 7.68 (s, 1H), 4.21 (q, J=6.8 Hz, 2H), 1.32 (t, J=6.8 Hz, 3H).

### Preparation 14

### 2-bromo-5-(2,2,2-trifluoroethoxy)isonicotinic acid

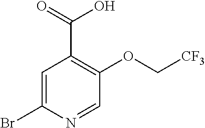 [SMILES: O=C(O)c1cc(Br)ncc1OCC(F)(F)F]

To a solution of 2-bromo-5-fluoroisonicotinic acid (2.0 g, 9.09 mmol) in THF (20.0 mL) was added 2,2,2-trifluoroethan-1-ol (2.7 g, 27.27 mmol) and t-BuONa (4.4 g, 45.46 mmol) and the reaction was stirred at 70° C. for 12 h under N₂. The mixture was concentrated under reduced pressure, the residue was diluted with water (20 mL) and the pH adjusted to 4 with 12 M HCl. The mixture was filtered, the filter cake was washed with water (10 mL×2) and dried under reduced pressure to give 2-bromo-5-(2,2,2-trifluoroethoxy)isonicotinic acid (2.4 g, 88% yield) as a white solid. ¹H NMR: (500 MHz, DMSO-d₆) δ ppm: 13.88 (br s, 1H), 8.44 (s, 1H), 7.80 (s, 1H), 4.97 (q, J=8.5 Hz, 2H).

### Preparation 15

### 2-bromo-5-(2,2-difluoroethoxy)isonicotinic acid

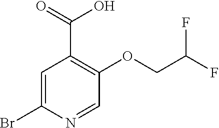 [SMILES: O=C(O)c1cc(Br)ncc1OCC(F)F]

2-Bromo-5-(2,2-difluoroethoxy)isonicotinic acid was obtained, 1.5 g, 58.5% yield as a brown solid, from 2-bromo-5-fluoroisonicotinic acid and 2,2-difluoroethan-1-ol following the procedure described in Preparation 14. ¹H NMR: (400 MHz, MeOH-d₄) δ ppm: 8.32 (s, 1H), 7.78 (s, 1H), 6.38-6.10 (m, 1H), 4.47-4.39 (m, 2H).

### Preparation 16

### 2-bromo-5-(2-(dimethylamino)ethoxy)isonicotinic acid

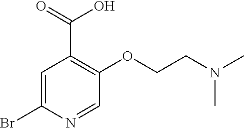 [SMILES: CN(C)CCOc1cnc(Br)cc1C(=O)O]

2-Bromo-5-(2-(dimethylamino)ethoxy)isonicotinic acid was obtained as a yellow solid, 2.03 g, 76.9%, from 2-bromo-5-fluoroisonicotinic acid and 2-(dimethylamino)ethan-1-ol, following the procedure described in Preparation 14. LCMS m/z=291.0 [M+H]⁺

### Preparation 17

### 5-(benzyloxy)-2-bromoisonicotinic acid

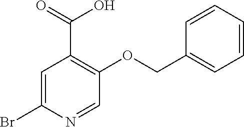 [SMILES: O=C(O)c1cc(Br)ncc1OCc1ccccc1]

5-(Benzyloxy)-2-bromoisonicotinic acid was obtained as a yellow solid, 25 g, 93.9% yield, from 2-bromo-5-fluoroisonicotinic acid and benzyl alcohol following the procedure described in Preparation 14. LCMS m/z=307.9 [M+H]⁺.

### Preparation 18

### 2-bromo-5-((3-methoxycyclobutyl)methoxy)isonicotinic acid

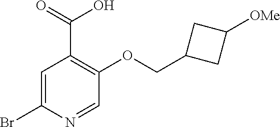 [SMILES: COC1CC(COc2cnc(Br)cc2C(=O)O)C1]

To a solution of 2-bromo-5-fluoroisonicotinic acid (1 g, 4.55 mmol) in THF (10 mL) was added (3-methoxycyclobutyl)methanol (580.80 mg, 5.0 mmol), t-BuONa (873.68 mg, 9.09 mmol) and the reaction was stirred at 70° C. for 2 h. The mixture was concentrated and purified by Prep-HPLC (Method E, gradient: 25 to 55%) to give 2-bromo-5-((3-methoxycyclobutyl)methoxy)isonicotinic acid (340 mg, 23.7% yield) as a white solid. ¹H NMR: (400 MHz, CDCl₃) δ ppm 8.26 (s, 1H), 8.10 (s, 1H), 4.31 (d, J=6.4 Hz, 2H), 3.92-3.85 (m, 1H), 3.26 (s, 3H), 2.56-2.46 (m, 3H), 1.89-1.79 (m, 2H).

### Preparation 19

### 2-bromo-5-(cyclopropylmethoxy)isonicotinic acid

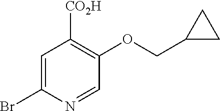 [SMILES: O=C(O)c1cc(Br)ncc1OCC1CC1]

To a solution of 2-bromo-5-fluoroisonicotinic acid (19 g, 86.37 mmol) and cyclopropylmethanol (12.45 g, 172.73 mmol) in THF (250 mL) was added t-BuONa (20.75 g, 215.9 mmol). The reaction mixture was stirred at 90° C. for 16 h. The reaction was concentrated to give the residue, which was adjusted with HCl (2 N) until pH to 5. The mixture was filtered and the solid was dried in vacuo to give 2-bromo-5-(cyclopropylmethoxy)isonicotinic acid (20 g, 85.1% yield) as a white solid. 1H NMR: (400 MHz, CDCl₃) δ ppm: 13.66 (br s, 1H), 8.31 (s, 1H), 7.69 (s, 1H), 4.03 (d, J=7.2 Hz, 2H), 1.30-1.13 (m, 1H), 0.62-0.51 (m, 2H), 0.40-0.30 (m, 2H)

### Preparation 20

### 2-bromo-5-(ethoxy-d₅)isonicotinic acid

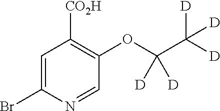 [SMILES: [2H]C([2H])([2H])C([2H])([2H])Oc1cnc(Br)cc1C(=O)O]

To a solution of t-BuONa (2.18 g, 22.73 mmol) and THF (20 mL) was added 1,1,1,2,2-pentadeuterio-2-deuteriooxy-ethane (1.2 g, 23.03 mmol) while stirring at rt. After 5 mins, 2-bromo-5-fluoro-pyridine-4-carboxylic acid (2 g, 9.09 mmol) was added, then the reaction was warmed to 65° C. The reaction was allowed to stir for 1 h, then diluted with water (40 mL water) and concentrated to remove THF. The resulting aqueous mixture was acidified by the addition of HCl (2 M, 13.64 mL) while stirring at rt. The thick aqueous slurry was filtered to collect the resulting solids. These were then dissolved in MeOH (50 mL), diluted with MeCN (25 mL) and concentrated to give, 2-bromo-5-(ethoxy-d5)isonicotinic acid as a tan solid (2.2 g, 96.4% yield). LCMS m/z=250.9 (M+H)+. ¹H NMR (DMSO-d₆) δ: 14.14-13.10 (m, 1H), 8.32 (s, 1H), 7.68 (s, 1H).

### Preparation 21

### tert-butyl (2-bromo-5-(cyclopropylmethoxy)pyridin-4-yl)carbamate

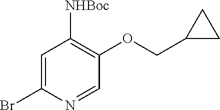 [SMILES: CC(C)(C)OC(=O)Nc1cc(Br)ncc1OCC1CC1]

To a solution of 2-bromo-5-(cyclopropylmethoxy)isonicotinic acid (Preparation 19, 20 g, 73.50 mmol) and TEA (8.93 g, 88.20 mmol) in t-BuOH (200 mL) was added DPPA (19.01 mL, 88.20 mmol) and the reaction was stirred at 90° C. for 16 h. The mixture was partitioned between EtOAc (3×150 mL) and water (250 mL). The organic layer was washed with brine (300 mL), dried over Na₂SO₄, filtered and concentrated to give the residue, which was purified by column chromatography on silica gel (PE/EtOAc=20/1-10/1) to give tert-butyl (2-bromo-5-(cyclopropylmethoxy)pyridin-4-yl)carbamate (22 g, 87.2% yield) as a yellow solid. ¹HNMR: (400 MHz, CDCl₃) δ ppm: 8.23 (s, 1H), 7.81 (s, 1H), 3.90 (d, J=6.8 Hz, 2H), 1.55 (s, 9H), 1.38-1.22 (m, 1H), 0.80-0.61 (m, 2H), 0.47-0.30 (m, 2H).

### Preparation 22

### tert-butyl (2-bromo-5-(2-methoxyethoxy)pyridin-4-yl)carbamate

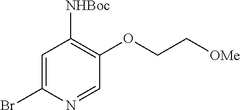 [SMILES: COCCOc1cnc(Br)cc1NC(=O)OC(C)(C)C]

To a solution of 2-bromo-5-(2-methoxyethoxy)isonicotinic acid (Preparation 12, 10.0 g, 36.2 mmol) in t-BuOH (100 mL) was added TEA (7.3 g, 72.4 mmol) and DPPA (9.9 g, 36.2 mmol) and the reaction was stirred at 90° C. for 16 h under N₂. The mixture was concentrated under reduced pressure, the residue diluted with H₂O (30 mL) and extracted with EtOAc (35 mL×3). The combined organic layers were washed with brine (35 mL), dried over Na₂SO₄, filtered and concentrated under reduced pressure. The crude product was purified by chromatography on silica gel (PE/EtOAc 1/0 to 5/1) to give tert-butyl (2-bromo-5-(2-methoxyethoxy)pyridin-4-yl)carbamate (9.03 g, 71.8% yield) as a white crystal. LCMS m/z=349.1 [M+H]⁺.

### Preparation 23

### tert-butyl (2-bromo-5-ethoxypyridin-4-yl)carbamate

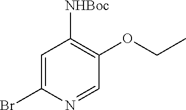 [SMILES: CCOc1cnc(Br)cc1NC(=O)OC(C)(C)C]

tert-Butyl (2-bromo-5-ethoxypyridin-4-yl)carbamate was obtained as a yellow solid, 85 g, 73.3% yield, from 2-bromo-5-ethoxyisonicotinic acid (Preparation 13) and DPPA, following the procedure described in Preparation 22. 1H NMR: (400 MHz, DMSO-d₆) δ ppm 8.60 (s, 1H), 8.03 (s, 1H), 8.02 (s, 1H), 4.16 (q, J=6.8 Hz, 2H), 1.49 (s, 9H), 1.38 (t, J=6.8 Hz, 3H).

### Preparation 24 to 29

The compounds in the following table were obtained from the appropriate 2-bromoisonicotinic acid and DPPA, following a similar reaction to that described in Preparation 22.

| Preparation | |
|---|---|
| No | Name, Structure, Starting Material (SM), Data |
| 24 | tert-butyl (2-bromo-5-methoxypyridin-4-yl)carbamate |
| | 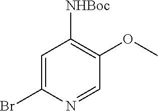 [SMILES: COc1cnc(Br)cc1NC(=O)OC(C)(C)C] |
| | SM: 2-bromo-5-methoxyisonicotinic acid |
| | 17 g, 65.1% yield as yellow solid. LCMS m/z = |
| | 303.1 [M + H]⁺ |
| 25 | tert-butyl (2-bromo-5-cyclopropoxypyridin-4-yl)carbamate |
| | 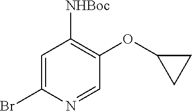 [SMILES: CC(C)(C)OC(=O)Nc1cc(Br)ncc1OC1CC1] |
| | SM: 2-bromo-5-cyclopropoxyisonicotinic acid |
| | (WO2021032148, Example 161, step 1) |
| | 406 mg, 51.3% yield) as a white solid. ¹H NMR |
| | (400 MHz, CDCl₃) δ ppm: |
| | 8.21 (s, 1H), 8.17 (s, 1H), 7.04 (s, 1H), |
| | 3.90-3.78 (m, 1H), 1.53 (s, 9H), 0.88- |
| | 0.83 (m, 4H). |
| 26 | tert-butyl (2-bromo-5-((3-methoxycyclobutyl)methoxy) |
| | pyridin-4-yl)carbamate |
| | 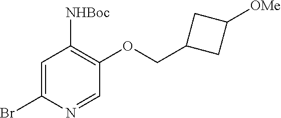 [SMILES: COC1CC(COc2cnc(Br)cc2NC(=O)OC(C)(C)C)C1] |
| | SM: 2-bromo-5-((3-methoxycyclobutyl)methoxy) |
| | isonicotinic acid (Preparation 18) |
| | yellow solid, 265 mg, 72.1%. 1H NMR: (500 MHz, CDCl₃) |
| | δ ppm 8.24 (s, 1H), 7.81 (s, 1H), 7.40 (s, 1H), 4.05-4.02 |
| | (m, 2H), 3.89-3.83 (m, 1H), 3.26 (s, 3H), 2.52-2.48 |
| | (m, 2H), 2.45-2.37 (m, 1H), 1.88-1.81 (m, 2H), 1.54 (s, |
| | 9H). |
| 27 | tert-butyl (2-bromo-5-(2,2,2-trifluoroethoxy)pyridin-4-yl) |
| | carbamate |
| | 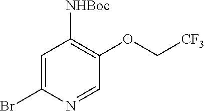 [SMILES: CC(C)(C)OC(=O)Nc1cc(Br)ncc1OCC(F)(F)F] |
| | SM: 2-bromo-5-(2,2,2-trifluoroethoxy)isonicotinic acid |
| | (Preparation 14) |
| | white solid, 2.8 g, 94% yield, 1H NMR: (400 MHz, |
| | CDCl₃) δ ppm: 8.32 (s, 1H), 7.88 (s, 1H), 7.08 |
| | (s, 1H), 4.47 (q, J-8.0 Hz, 2H), 1.54 (s, 9H). |
| 28 | tert-butyl (2-bromo-5-(2,2-difluoroethoxy)pyridin-4-yl) |
| | carbamate |
| | 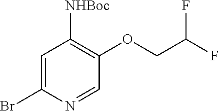 [SMILES: CC(C)(C)OC(=O)Nc1cc(Br)ncc1OCC(F)F] |
| | SM: 2-bromo-5-(2,2-difluoroethoxy)isonicotinic acid |
| | (Preparation 15) |
| | light yellow solid, 525 mg, 41.9% yield, 1H NMR: (500 |
| | MHz, CDCl₃) δ ppm: 8.29 (s, 1H), 7.87 (s, 1H), |
| | 6.15-6.03 (m, 1H), 4.33-4.23 (m, 2H), 1.54 |
| | (s, 9H) |
| 29 | tert-butyl (5-(benzyloxy)-2-bromopyridin-4-yl) |
| | carbamate |
| | 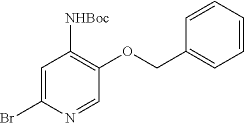 [SMILES: CC(C)(C)OC(=O)Nc1cc(Br)ncc1OCc1ccccc1] |
| | SM: 5-(benzyloxy)-2-bromoisonicotinic acid |
| | (Preparation 17) |
| | 22 g, 71.50% yield as yellow solid. LCMS m/z = 380.1 |
| | [M + H]⁺. |

### Preparation 30

### tert-butyl (2-bromo-5-(2-(dimethylamino)ethoxy)pyridin-4-yl)carbamate

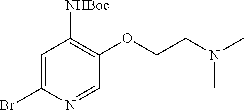 [SMILES: CN(C)CCOc1cnc(Br)cc1NC(=O)OC(C)(C)C]

To a solution of 2-bromo-5-(2-(dimethylamino)ethoxy)isonicotinic acid (Preparation 16, 2 g, 6.92 mmol) in t-BuOH (20 mL) was added TEA (1.40 g, 13.83 mmol) and DPPA (1.90 g, 6.92 mmol) and the reaction was stirred at 90° C. for 16 h under N₂. The reaction mixture was purified by prep-HPLC (Method D, Gradient 20% isocratic) to give tert-butyl (2-bromo-5-(2-(dimethylamino)ethoxy)pyridin-4-yl)carbamate (800 mg, 32.1% yield) as a white solid. LCMS m/z=361.8 [M+H]⁺

### Preparation 31

### tert-butyl (2-bromo-5-(ethoxy-d₅)pyridin-4-yl)carbamate

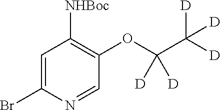 [SMILES: [2H]C([2H])([2H])C([2H])([2H])Oc1cnc(Br)cc1NC(=O)OC(C)(C)C]

To a mixture of 2-bromo-5-(ethoxy-d5)isonicotinic acid (Preparation 20, 2.2 g, 8.76 mmol) in tBuOH (25 mL) was added DIPEA (2.26 g, 17.52 mmol), the solution stirred at rt for 10 mins, then DPPA (2.89 g, 10.51 mmol) was added. After 5 mins, the reaction was warmed to 75° C. with the cap loose to allow for N₂ evolution for 2 h. The cooled reaction was concentrated to remove tBuOH, then diluted with NaHCO₃ (50 mL) and extracted with EtOAc (3×50 mL). The combined organic layers were concentrated to dryness and the crude material was purified by silica gel chromatography (40 g, heptane to EtOAc) to give tert-butyl (2-bromo-5-(ethoxy-d₅)pyridin-4-yl)carbamate as a white crystalline solid, (1.42 g, 50.30% yield). LCMS m/z=322.0 [M+H]⁺. 1H NMR (DMSO-d₆) δ: 8.62 (br s, 1H), 8.03 (d, J=4.9 Hz, 2H), 1.49 (s, 9H).

### Preparation 32

### tert-butyl (2-acetamido-5-(2-methoxyethoxy)pyridin-4-yl)carbamate

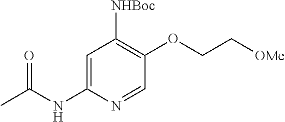 [SMILES: COCCOc1cnc(NC(C)=O)cc1NC(=O)OC(C)(C)C]

To a solution of tert-butyl (2-bromo-5-(2-methoxyethoxy)pyridin-4-yl)carbamate (Preparation 22, 8.9 g, 25.6 mmol) and acetamide (7.6 g, 128 mmol) in dioxane (80 mL) was added Cs₂CO₃ (25.1 g, 76.9 mmol) and BrettPhos Pd G3 (2.3 g, 2.56 mmol) and the reaction was stirred at 100° C. for 1 h under N₂. The mixture was concentrated, diluted with H₂O (50 mL), extracted with EtOAc (60 mL×2), and the combined organic extracts washed with brine (50 mL×2). The organic phase was concentrated and the residue purified by chromatography on silica gel (PE/EtOAc 5/0 to 0/1) to give tert-butyl (2-acetamido-5-(2-methoxyethoxy)pyridin-4-yl)carbamate (8.0 g, 95.9% yield) as a brown solid. LCMS m/z=326.2 [M+H]⁺. ¹H NMR: (400 MHz, CDCl₃) δ ppm: 8.88 (br s, 1H), 8.27 (br s, 1H), 7.83-7.79 (m, 2H), 4.14-4.10 (m, 2H), 3.70-3.67 (m, 2H), 3.46 (s, 3H), 2.15 (s, 3H), 1.54 (s, 9H).

### Preparation 33

### tert-butyl (2-acetamido-5-(cyclopropylmethoxy)pyridin-4-yl)carbamate

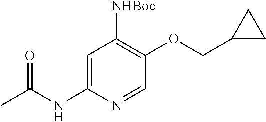 [SMILES: CC(=O)Nc1cc(NC(=O)OC(C)(C)C)c(OCC2CC2)cn1]

To a solution of tert-butyl (2-bromo-5-(cyclopropylmethoxy)pyridin-4-yl)carbamate (Preparation 21, 150 mg, 0.437 mmol) in dioxane (5 mL) was added acetamide (77.4 mg, 1.31 mmol), BrettPhos Pd G3 (39.6 mg, 0.0437 mmol) and Cs₂CO₃ (427 mg, 1.31 mmol). The reaction was stirred at 100° C. for 2 h under N₂. The mixture was diluted with H₂O (10 mL) and extracted with EtOAc (15 mL×3). The organic phase was washed with brine (20 mL), dried over Na₂SO₄, filtered, concentrated, then purified by chromatography on silica gel (PE/EtOAc 15/1 to 0/1) to give tert-butyl (2-acetamido-5-(cyclopropylmethoxy)pyridin-4-yl)carbamate (130 mg, 92.6% yield) as a white solid. ¹H NMR: (400 MHz, CDCl₃) δ ppm: 8.85 (br s, 1H), 7.89 (br s, 1H), 7.71 (s, 1H), 3.87 (d, J=7.2 Hz, 2H), 2.16 (s, 3H), 1.56 (s, 9H), 1.30-1.25 (m, 1H), 0.70-0.66 (m, 2H), 0.38-0.35 (m, 2H).

### Preparations 34 to 39

The compounds in the following table were prepared from the appropriate 2-bromo or 2-chloropyridine and acetamide, following a similar procedure to that described in Preparation 33.

| Preparation | |
|---|---|
| No | Name, Structure, Starting material (SM), Data |
| 34 | tert-butyl (2-acetamido-5-(difluoromethoxy)pyridin-4-yl)carbamate |
| | 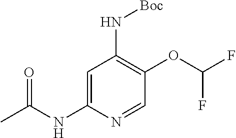 [SMILES: CC(=O)Nc1cc(NC(=O)OC(C)(C)C)c(OC(F)F)cn1] |
| | SM: tert-butyl (2-chloro-5-(difluoromethoxy)pyridin-4-yl)carbamate |
| | (Preparation 9) |
| | (750 mg, 75.7% yield) as a yellow oil. ¹H NMR: (500 MHz, CDCl₃) δ: ppm |
| | 8.98 (s, 1H), 8.00 (s, 1H), 7.93 (s, 1H), 7.01 (s, 1H), 6.50 (t, J = 73.0 Hz, 1H), |
| | 2.19 (s, 3H), 1.53 (s, 9H). |
| 35 | tert-butyl (2-acetamido-5-methoxypyridin-4-yl)carbamate |
| | 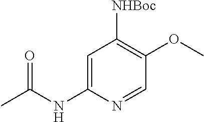 [SMILES: COc1cnc(NC(C)=O)cc1NC(=O)OC(C)(C)C] |
| | SM: tert-butyl (2-bromo-5-methoxypyridin-4-yl)carbamate (Preparation 24) |
| | 15 g, 95.1% yield, as yellow solid. LCMS m/z = 282.1 [M + H]⁺ |
| 36* | tert-butyl (2-acetamido-5-(2-(dimethylamino)ethoxy)pyridin-4- |
| | yl)carbamate |
| | 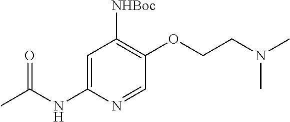 [SMILES: CC(=O)Nc1cc(NC(=O)OC(C)(C)C)c(OCCN(C)C)cn1] |
| | SM: tert-butyl (2-bromo-5-(2-(dimethylamino)ethoxy)pyridin-4- |
| | yl)carbamate (Preparation 30) |
| | 157 mg, 41.8% yield as a white solid. LCMS m/z = 339.2 [M + H]⁺ |
| 37** | tert-butyl (2-acetamido-5-cyclobutoxypyridin-4-yl)carbamate |
| | 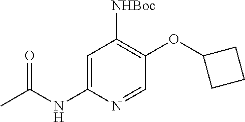 [SMILES: CC(=O)Nc1cc(NC(=O)OC(C)(C)C)c(OC2CCC2)cn1] |
| | SM: tert-butyl (2-chloro-5-cyclobutoxypyridin-4-yl)carbamate (Preparation |
| | 10) |
| | 60 mg, 27.9% yield as a white solid. LCMS m/z = 322.0 [M + H]⁺ |
| 38 | tert-butyl (2-acetamido-5-(benzyloxy)pyridin-4-yl)carbamate |
| | 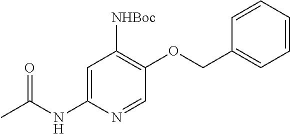 [SMILES: CC(=O)Nc1cc(NC(=O)OC(C)(C)C)c(OCc2ccccc2)cn1] |
| | SM: tert-butyl (5-(benzyloxy)-2-bromopyridin-4-yl)carbamate (Preparation |
| | 29) |
| | 16 g, 84.9% yield) as yellow solid. LCMS m/z = 358.2 [M + H]⁺. ¹HNMR |
| | (400 MHz, DMSO-d₆) δ ppm 10.17 (s, 1H), 8.59 (s, 1H), 8.17 (s, 1H), 7.97 |
| | (s, 1H), 7.50 (d, J = 7.2 Hz, 2H), 7.29-7.44 (m, 3H), 5.20 (s, 2H), 2.03 (s, |
| | 3H), 1.47 (s, 9H). |
| 39 | tert-butyl (2-acetamido-5-(2,2-difluoroethoxy)pyridin-4-yl)carbamate |
| | 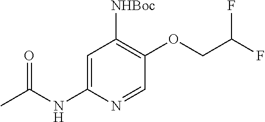 [SMILES: CC(=O)Nc1cc(NC(=O)OC(C)(C)C)c(OCC(F)F)cn1] |
| | SM: tert-butyl (2-bromo-5-(2,2-difluoroethoxy)pyridin-4-yl)carbamate |
| | (Preparation 28) |
| | light yellow solid, 307 mg, 62.9% yield. ¹H NMR (400 MHz, MeOH-d₄) δ |
| | ppm: 8.72 (s, 1H), 7.94 (s, 1H), 6.41-6.12 (m, 1H), 4.39-4.31 (m, 2H), 2.14 |
| | (s, 3H), 1.55 (s, 9H). |

*purified by prep TLC (DCM/MeOH = 10/1)
**DMF was used instead of dioxane

### Preparation 40

### tert-butyl (2-acetamido-5-(ethoxy-d5)pyridin-4-yl)carbamate

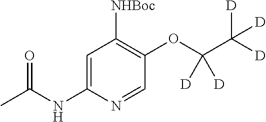 [SMILES: [2H]C([2H])([2H])C([2H])([2H])Oc1cnc(NC(C)=O)cc1NC(=O)OC(C)(C)C]

To tert-butyl (2-bromo-5-(ethoxy-d₅)pyridin-4-yl)carbamate (Preparation 31, 1.42 g, 4.41 mmol) was added acetamide (1.30 g, 22.04 mmol), Cs₂CO₃ (4.31 g, 13.22 mmol) and BrettPhos Pd G3 (300 mg, 330.9 μmol) followed by dioxane (15 mL), the mixture was degassed by bubbling N₂ for 10 mins at rt, then heated to 100° C. for 1 h. The reaction mixture was diluted with EtOAc (25 mL), filtered through Celite®, rinsing with EtOAc (15 mL), then concentrated to dryness. The crude material was purified by silica gel chromatography (40 g, heptane to EtOAc) to give tert-butyl (2-acetamido-5-(ethoxy-d5)pyridin-4-yl)carbamate (1.26 g, 94.9% yield) as a white solid. LCMS m/z=301.1 [M+H]+. 1H NMR (DMSO-d₆) δ: 10.19 (br s, 1H), 8.63 (br s, 1H), 8.11 (br s, 1H), 7.92 (s, 1H), 2.04 (s, 3H), 1.49 (s, 9H).

### Preparation 41

### tert-butyl (2-acetamido-5-isopropoxypyridin-4-yl)carbamate

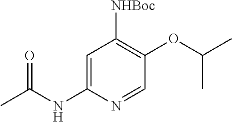 [SMILES: CC(=O)Nc1cc(NC(=O)OC(C)(C)C)c(OC(C)C)cn1]

To a solution of tert-butyl (2-chloro-5-isopropoxypyridin-4-yl)carbamate (Preparation 11, 1 g, 3.49 mmol) in dioxane (10 mL) was added acetamide (618 mg, 10.46 mmol), Pd₂(dba)₃ (638.68 mg, 0.7 mmol), Cs₂CO₃ (2.27 g, 6.97 mmol) and Xantphos (807.13 mg, 1.39 mmol) and the reaction was stirred at 120° C. for 16 h under N₂. The mixture was diluted with water (40 mL) and extracted with EtOAc (40 mL×3). The combined organic layers were washed with brine (40 mL), dried over Na₂SO₄, filtered and concentrated in vacuo. The residue was purified by silica gel column chromatography (PE/EtOAc=1/0 to 1/1) to give tert-butyl (2-acetamido-5-isopropoxypyridin-4-yl)carbamate (830 mg, 76.9% yield) as a yellow solid. ¹H NMR (500 MHz, CDCl₃) δ ppm: 8.83 (s, 1H), 7.95 (s, 1H), 7.76 (s, 1H), 7.15 (s, 1H), 4.60-4.52 (m, 1H), 2.16 (s, 3H), 1.56 (s, 9H), 1.38 (d, J=6.0 Hz, 6H)

### Preparation 42

### tert-butyl (2-acetamido-5-cyclopropoxypyridin-4-yl)carbamate

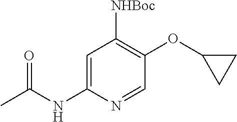 [SMILES: CC(=O)Nc1cc(NC(=O)OC(C)(C)C)c(OC2CC2)cn1]

tert-Butyl (2-acetamido-5-cyclopropoxypyridin-4-yl)carbamate was obtained as a yellow solid, 130 mg, 34.8% yield, from tert-butyl (2-bromo-5-cyclopropoxypyridin-4-yl)carbamate (Preparation 25) and acetamide following the procedure described in Preparation 41. LCMS m/z=308.1 [M+H]⁺

### Preparation 43

### tert-butyl (2-acetamido-5-(2,2,2-trifluoroethoxy)pyridin-4-yl)carbamate

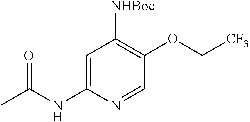 [SMILES: CC(=O)Nc1cc(NC(=O)OC(C)(C)C)c(OCC(F)(F)F)cn1]

tert-Butyl (2-acetamido-5-(2,2,2-trifluoroethoxy)pyridin-4-yl)carbamate was obtained as a white solid, 733 mg, 74% yield, from tert-butyl (2-bromo-5-(2,2,2-trifluoroethoxy)pyridin-4-yl)carbamate (Preparation 27) and acetamide following the procedure described in Preparation 41. ¹H NMR (400 MHz, CDCl₃) δ ppm: 8.93 (br s, 1H), 7.79 (s, 1H), 7.74 (s, 1H), 7.06 (s, 1H), 4.42 (q, J=8.0 Hz, 2H), 2.18 (s, 3H), 1.55 (s, 9H).

### Preparation 44

### tert-butyl (2-acetamido-5-((3-methoxycyclobutyl)methoxy)pyridin-4-yl)carbamate

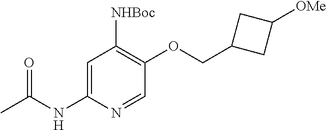 [SMILES: COC1CC(COc2cnc(NC(C)=O)cc2NC(=O)OC(C)(C)C)C1]

tert-Butyl (2-acetamido-5-((3-methoxycyclobutyl)methoxy)pyridin-4-yl)carbamate was obtained as a yellow oil, 98 mg, 43.3% yield, from tert-butyl (2-bromo-5-((3-methoxycyclobutyl)methoxy)pyridin-4-yl)carbamate (Preparation 26) and acetamide following the procedure described in Preparation 41. ¹H NMR: (500 MHz, CDCl₃) δ ppm 8.84 (s, 1H), 7.76 (s, 1H), 7.71 (s, 1H), 7.28 (s, 1H), 4.02 (d, J=5.5 Hz, 2H), 3.88-3.82 (m, 1H), 3.26 (s, 3H), 2.53-2.46 (m, 2H), 2.43-2.34 (m, 1H), 2.16 (s, 3H), 1.86-1.80 (m, 2H), 1.56 (s, 9H).

### Preparation 45

### tert-butyl (5-ethoxy-2-propionamidopyridin-4-yl)carbamate

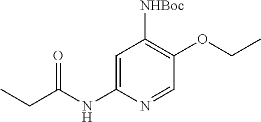 [SMILES: CCOc1cnc(NC(=O)CC)cc1NC(=O)OC(C)(C)C]

To a solution of tert-butyl (2-bromo-5-ethoxypyridin-4-yl)carbamate (Preparation 23, 400.0 mg, 1.26 mmol) and propionamide (460.9 mg, 6.31 mmol) in dioxane (5.0 mL) was added Cs₂CO₃ (1.2 g, 3.78 mmol) and BrettPhos Pd G₃ (114.3 mg, 0.13 mmol) and the reaction was stirred at 100° C. under N₂ for 1 h. The mixture was concentrated under reduced pressure to give the residue, which was purified by column chromatography (PE/EtOAc=3/1 to 0/1) to give tert-butyl (5-ethoxy-2-propionamidopyridin-4-yl)carbamate (255 mg, 65.4% yield) as a white solid. LCMS m/z=310.2 [M+H]⁺

### Preparation 46

### tert-butyl (5-ethoxy-2-(2-methoxyacetamido)pyridin-4-yl)carbamate

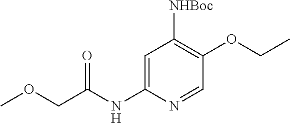 [SMILES: CCOc1cnc(NC(=O)COC)cc1NC(=O)OC(C)(C)C]

tert-Butyl (5-ethoxy-2-(2-methoxyacetamido)pyridin-4-yl)carbamate was obtained as a yellow solid, 600 mg, 82.2% yield from 2-methoxyacetamide and tert-butyl (2-bromo-5-ethoxypyridin-4-yl)carbamate (Preparation 23), following the procedure described in Preparation 45. ¹H NMR (400 MHz, CDCl₃) δ: ppm 8.90 (s, 1H), 8.68 (s, 1H), 7.77 (s, 1H), 7.18 (s, 1H), 4.13 (q, J=6.8 Hz, 2H), 4.01 (s, 2H), 3.48 (s, 3H), 1.55 (s, 9H), 1.47 (t, J=7.2 Hz, 3H).

### Preparation 47

### tert-butyl (5-ethoxy-2-(3-methoxypropanamido)pyridin-4-yl)carbamate

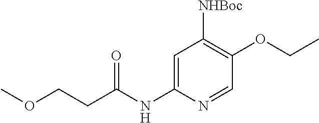 [SMILES: CCOc1cnc(NC(=O)CCOC)cc1NC(=O)OC(C)(C)C]

tert-Butyl (5-ethoxy-2-(3-methoxypropanamido)pyridin-4-yl)carbamate was obtained as a white solid, 372 mg, 86.9% yield, from 3-methoxypropanamide and tert-butyl (2-bromo-5-ethoxypyridin-4-yl)carbamate (Preparation 23) following a similar procedure to that described in Preparation 45. ¹H NMR (500 MHz, CDCl₃) δ ppm: 8.90 (br s, 1H), 8.49 (br s, 1H), 7.75 (s, 1H), 7.16 (s, 1H), 4.11-4.13 (m, 2H), 3.72 (t, J=6.0 Hz, 2H), 3.42 (s, 3H), 2.63 (t, J=6.0 Hz, 2H), 1.55 (s, 9H), 1.46 (t, J=7.0 Hz, 3H)

### Preparation 48

### tert-butyl (2-(cyclopropanecarboxamido)-5-ethoxypyridin-4-yl)carbamate

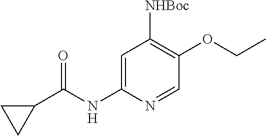 [SMILES: CCOc1cnc(NC(=O)C2CC2)cc1NC(=O)OC(C)(C)C]

To a solution of tert-butyl (2-bromo-5-ethoxypyridin-4-yl)carbamate (Preparation 23, 200 mg, 0.631 mmol), Pd₂(dba)₃ (57.8 mg, 0.063 mmol), Xantphos (36.5 mg, 0.063 mmol) and K₃PO₄ (267.7 mg, 1.26 mmol) in dioxane (8.0 mL) was added cyclopropanecarboxamide (64.4 mg, 0.757 mmol) and the reaction was stirred at 100° C. for 2 h under N₂. The cooled reaction mixture was filtered and the filtrate concentrated under reduced pressure. The residue was purified by silica gel column chromatography (PE/EtOAc=1/0 to 1/1) to give tert-butyl (2-(cyclopropanecarboxamido)-5-ethoxypyridin-4-yl)carbamate (210 mg) as a yellow solid. LCMS m/z=322.1 [M+H]⁺

### Preparation 49

### 2,4-dichloro-5-(methoxymethyl)pyridine

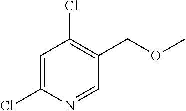

To a stirring solution of (4,6-dichloro-3-pyridyl)methanol (300 mg, 1.69 mmol) in THF (9 mL) was added NaH (60.66 mg, 2.53 mmol) at 0° C. The mixture was stirred at rt for 15 mins, then iodomethane (358.80 mg, 2.53 mmol) was added. The reaction mixture was stirred at rt for 4 h then quenched with aq. NH₄Cl and was extracted with EtOAc. The combined organic extracts were concentrated in vacuo and the crude product was purified (0-100% heptane/EtOAc-ethanol (3:1)) to obtain 2,4-dichloro-5-(methoxymethyl)pyridine (225 mg, 69.5% yield) as colorless liquid. LCMS m/z=192.0 [M+H]⁺.

### Preparation 50

### N-(4-chloro-5-(methoxymethyl)pyridin-2-yl)acetamide

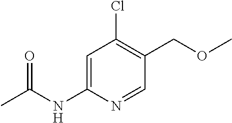

To a stirring solution of 2,4-dichloro-5-(methoxymethyl)pyridine (Preparation 49, 317 mg, 1.65 mmol) and acetamide (97.50 mg, 1.65 mmol) in toluene (2 mL, degassed for 20 mins using N₂) were added Josiphos Pd G3 (76.28 mg, 0.083 mmol) and K₂CO₃ (456.27 mg, 3.30 mmol). The resulting mixture was degassed using N₂ then heated at 90° C. for 16 h. The reaction mixture was filtered through Celite® and the crude product was purified by silica gel column chromatography using (0-100%) heptane/EtOAc/EtOH (3:1) to obtain N-(4-chloro-5-(methoxymethyl)pyridin-2-yl)acetamide (211 mg, 59.6% yield) as an orange colored solid. LCMS m/z=214.0 [M+H]⁺.

### Preparation 51

### N-(4-chloro-5-methylpyridin-2-yl)acetamide

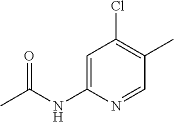

To a solution of 4-chloro-5-methylpyridin-2-amine (100 mg, 0.70 mmol) in DCM (2 mL) was added Ac₂O (85.92 mg, 0.84 mmol) and pyridine (77.67 mg, 0.98 mmol) and the mixture was stirred at 25° C. for 16 h. The mixture was added to water (10 mL) and extracted with DCM (15 mL×3). The combined organic layers were washed with brine (15 mL), dried over Na₂SO₄, filtered and concentrated in vacuo. The crude was purified by silica gel column chromatography (PE/EtOAc=1/0 to 3/1) to give N-(4-chloro-5-methylpyridin-2-yl)acetamide (98 mg, 75.7% yield) as a yellow solid. ¹H NMR: (500 MHz, CDCl₃) δ ppm: 9.69 (br s, 1H), 8.38 (s, 1H), 7.97 (s, 1H), 2.30 (s, 3H), 2.11 (s, 3H).

### Preparation 52

### tert-butyl (2-acetamido-5-methylpyridin-4-yl)carbamate

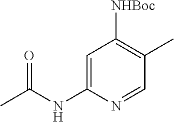 [SMILES: CC(=O)Nc1cc(NC(=O)OC(C)(C)C)c(C)cn1]

To a solution of N-(4-chloro-5-methylpyridin-2-yl)acetamide (Preparation 51, 90 mg, 0.49 mmol) in dioxane (1 mL) was added NH₂Boc (85.66 mg, 0.73 mmol), Cs₂CO₃ (317.66 mg, 0.97 mmol), Xantphos (112.83 mg, 0.19 mmol) and Pd₂(dba)₃ (89.28 mg, 0.10 mmol) and the mixture was stirred at 120° C. for 16 h under N₂. The mixture was added to water (10 mL) and extracted with EtOAc (15 mL×3). The combined organic layers were washed with brine (15 mL), dried over Na₂SO₄, filtered and concentrated in vacuo. The residue was purified by silica gel column chromatography (PE/EtOAc=1/0 to 0/1) to give tert-butyl (2-acetamido-5-methylpyridin-4-yl)carbamate (90 mg, 69.6% yield) as a yellow solid. 1H NMR: (500 MHz, CDCl₃) δ ppm: 8.81 (br s, 1H), 7.92 (s, 1H), 7.26 (s, 1H), 6.43 (s, 1H), 2.18 (s, 3H), 2.15 (s, 3H), 1.55 (s, 9H).

### Preparation 53

### tert-butyl (2-acetamido-5-bromopyridin-4-yl)carbamate

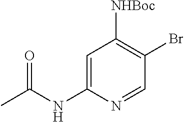 [SMILES: CC(=O)Nc1cc(NC(=O)OC(C)(C)C)c(Br)cn1]

To a solution of tert-butyl (2-acetamidopyridin-4-yl)carbamate (8.4 g, 33.43 mmol) in MeCN (90.0 mL) was added NBS (6.5 g, 36.77 mmol) and the reaction stirred at 70° C. for 1 h. The mixture was concentrated under vacuum and the crude was purified by column chromatography (PE/EtOAc=1/1 to 0/1) to give tert-butyl (2-acetamido-5-bromopyridin-4-yl)carbamate (9.1 g, 82.5% yield) as a white solid. ¹H NMR (500 MHz, CDCl₃) δ ppm: 9.07 (s, 1H), 8.28 (s, 1H), 8.20 (s, 1H), 7.14 (s, 1H), 2.21 (s, 3H), 1.55 (s, 9H).

### Preparation 54

### tert-butyl (2-acetamido-5-vinylpyridin-4-yl)carbamate

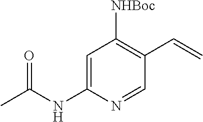 [SMILES: C=Cc1cnc(NC(C)=O)cc1NC(=O)OC(C)(C)C]

To a solution of tert-butyl (2-acetamido-5-bromopyridin-4-yl)carbamate (Preparation 53, 500 mg, 1.51 mmol) in dioxane (5.0 mL) and H₂O (1.0 mL) was added 4,4,5,5-tetramethyl-2-vinyl-1,3,2-dioxaborolane (466.5 mg, 3.03 mmol), Pd(dppf)Cl₂ (110.8 mg, 0.151 mmol) and K₂CO₃ (418.6 mg, 3.03 mmol) and the reaction was stirred at 100° C. for 2 h under N₂. The mixture was poured into H₂O (50 mL), extracted with EtOAc (50 mL×3), the combined organic layer was washed with brine (30 mL×3), dried over anhydrous Na₂SO₄, filtered and concentrated. The residue was purified by chromatography on silica gel (PE/EtOAc=20/1 to 1/1) to give tert-butyl (2-acetamido-5-vinylpyridin-4-yl)carbamate (381.6 mg, 90.9% yield) as a yellow solid. ¹H NMR (500 MHz, CDCl₃) δ ppm: 8.84 (s, 1H), 8.15 (s, 1H), 8.11 (s, 1H), 6.71 (s, 1H), 6.62 (dd, J=17.5 Hz, 11.0 Hz, 1H), 5.64 (d, J=17.5 Hz, 1H), 5.47 (d, J=11.0 Hz, 1H), 2.19 (s, 3H), 1.54 (s, 9H).

### Preparation 55

### tert-butyl (2-acetamido-5-(prop-1-en-2-yl)pyridin-4-yl)carbamate

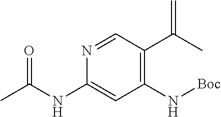 [SMILES: C=C(C)c1cnc(NC(C)=O)cc1NC(=O)OC(C)(C)C]

tert-Butyl (2-acetamido-5-(prop-1-en-2-yl)pyridin-4-yl)carbamate was obtained as a yellow solid, 220 mg, 83.1% yield, from tert-butyl (2-acetamido-5-bromopyridin-4-yl)carbamate (Preparation 53) and 4,4,5,5-tetramethyl-2-(prop-1-en-2-yl)-1,3,2-dioxaborolane, following the procedure described in Preparation 54. 1H NMR (400 MHz, DMSO-d₆) δ ppm: 10.40 (s, 1H), 8.44 (s, 1H), 8.31 (s, 1H), 8.02 (s, 1H), 5.28 (s, 1H), 5.00 (s, 1H), 2.06 (s, 3H), 1.06 (s, 9H).

### Preparation 56

### tert-butyl (2-acetamido-5-(prop-1-yn-1-yl)pyridin-4-yl)carbamate

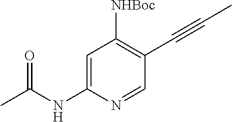 [SMILES: CC#Cc1cnc(NC(C)=O)cc1NC(=O)OC(C)(C)C]

To a solution of tert-butyl (2-acetamido-5-bromopyridin-4-yl)carbamate (Preparation 53, 500 mg, 1.51 mmol) in DMF (6.0 mL) was added tributyl(prop-1-yn-1-yl)stannane (747.6 mg, 2.27 mmol), NaOAc (62.1 mg, 0.757 mmol) and Pd(PPh₃)₂Cl₂ (53.2 mg, 0.076 mmol) and the reaction was stirred at 100° C. for 3 h under N₂. The mixture was poured into H₂O (30 mL), extracted with EtOAc (30 mL×3), the combined organic layer was washed with brine (20 mL×2), dried over anhydrous Na₂SO₄, filtered and concentrated in vacuo. The residue was purified by chromatography on silica gel (PE/EtOAc=20/1 to 1/1) to give tert-butyl (2-acetamido-5-(prop-1-yn-1-yl)pyridin-4-yl)carbamate (260.8 mg, 59.5% yield) as a white solid. ¹H NMR (400 MHz, CDCl₃) δ ppm: 8.90 (br s, 1H), 8.13 (s, 2H), 7.29 (s, 1H), 2.18 (s, 3H), 2.15 (s, 3H), 1.56 (s, 9H).

### Preparation 57

### tert-butyl (2-acetamido-5-ethylpyridin-4-yl)carbamate

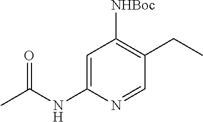 [SMILES: CCc1cnc(NC(C)=O)cc1NC(=O)OC(C)(C)C]

To a solution of tert-butyl (2-acetamido-5-vinylpyridin-4-yl)carbamate (Preparation 54, 361.8 mg, 1.30 mmol) in MeOH (5.0 mL) was added Pd/C (1.4 g, 1.30 mmol, 10% purity) and the reaction was stirred at 20° C. for 2 h under H₂ (15 psi). The reaction was filtered and concentrated in vacuo to give tert-butyl (2-acetamido-5-ethylpyridin-4-yl)carbamate (262.1 mg, 71.9% yield) as a white solid.

¹H NMR: (400 MHz, CDCl₃) δ ppm: 8.81 (s, 1H), 8.11 (s, 1H), 7.94 (s, 1H), 6.50 (s, 1H), 2.52 (q, J=7.6 Hz, 2H), 2.17 (s, 3H), 1.55 (s, 9H), 1.24 (t, J=7.6 Hz, 3H).

### Preparation 58

### tert-butyl (2-acetamido-5-isopropylpyridin-4-yl)carbamate

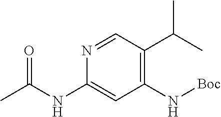 [SMILES: CC(=O)Nc1cc(NC(=O)OC(C)(C)C)c(C(C)C)cn1]

To a solution of tert-butyl (2-acetamido-5-(prop-1-en-2-yl)pyridin-4-yl)carbamate (Preparation 55, 200 mg, 0.69 mmol) in MeOH (5 mL) was added Pd/C (730.5 mg, 0.69 mmol, 10% purity) and the mixture was stirred at 25° C. for 16 h under H₂ (15 psi). The mixture was filtered and the filtrate was concentrated under pressure. The residue was diluted with water (10 mL) and extracted with EtOAc (15 mL×3). The combined organic layers were washed with brine (15 mL), dried over Na₂SO₄, filtered and concentrated in vacuo. The residue was purified by silica gel column chromatography (PE/EtOAc=1/0 to 1/2) to give tert-butyl (2-acetamido-5-isopropylpyridin-4-yl)carbamate (120 mg, 59.6% yield) as a white solid. 1H NMR (500 MHz, CDCl₃) δ ppm: 8.80 (s, 1H), 8.02 (s, 1H), 7.93 (s, 1H), 6.56 (s, 1H), 2.90-2.82 (m, 1H), 2.18 (s, 3H), 1.54 (s, 9H), 1.30 (d, J=6.5 Hz, 6H)

### Preparation 59

### tert-butyl (2-acetamido-5-propylpyridin-4-yl)carbamate

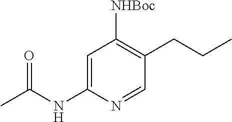 [SMILES: CCCc1cnc(NC(C)=O)cc1NC(=O)OC(C)(C)C]

To a solution of tert-butyl (2-acetamido-5-(prop-1-yn-1-yl)pyridin-4-yl)carbamate (Preparation 56, 260.8 mg, 0.901 mmol) in THF (5.0 mL) was added Pd/C (959.3 mg, 0.901 mmol, 10% purity) and the reaction was stirred at 20° C. for 2 h under H₂ (15 psi). The reaction was filtered and the filtrate concentrated to give tert-butyl (2-acetamido-5-propylpyridin-4-yl)carbamate (223.6 mg, 84.6% yield) as a white solid. ¹H NMR (400 MHz, CDCl₃) δ ppm: 8.81 (br s, 1H), 8.10 (s, 1H), 7.91 (s, 1H), 6.49 (s, 1H), 2.46 (t, J=7.6 Hz, 2H), 2.17 (s, 3H), 1.64-1.59 (m, 2H), 1.55 (s, 9H), 0.98 (t, J=7.2 Hz, 3H).

### Preparation 60

### tert-butyl (2-acetamido-5-cyclopropylpyridin-4-yl)carbamate

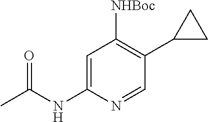 [SMILES: CC(=O)Nc1cc(NC(=O)OC(C)(C)C)c(C2CC2)cn1]

To a solution of tert-butyl (2-acetamido-5-bromopyridin-4-yl)carbamate (Preparation 53, 200.0 mg, 0.606 mmol) in dioxane (5.0 mL) and H₂O (0.5 mL) was added cyclopropylboronic acid (78.1 mg, 0.909 mmol), K₂CO₃ (251.2 mg, 1.82 mmol) and Pd(dppf)Cl₂ (44.3 mg, 0.061 mmol) and the reaction was stirred at 100° C. for 2 h. The mixture was concentrated in vacuo and the residue purified by column chromatography on silica gel (PE/EtOAc=5/1 to 0/1) to give tert-butyl (2-acetamido-5-cyclopropylpyridin-4-yl)carbamate (160 mg, 90.7% yield) as yellow oil. LCMS m/z=292.1 [M+H]⁺

### Preparation 61

### N-(4-amino-5-methoxypyridin-2-yl)acetamide hydrochloride

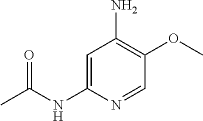

To a solution of tert-butyl (2-acetamido-5-methoxypyridin-4-yl)carbamate (Preparation 35, 15 g, 53.32 mmol) in dioxane (200 mL) was added 4M HCl/dioxane (200 mL) and the reaction was stirred at 20° C. for 1 h. The mixture was filtered and the filter cake was dried in vacuo followed by lyophilization to yield N-(4-amino-5-methoxypyridin-2-yl)acetamide hydrochloride (10.36 g, 84.21% yield) as yellow solid. LCMS m/z=182 [M+H]⁺

### Preparation 62N-(4-amino-5-ethoxypyridin-2-yl)acetamide hydrochloride

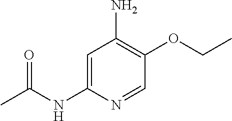

To a solution of tert-butyl (2-acetamido-5-ethoxypyridin-4-yl)(tert-butoxycarbonyl)carbamate (Preparation 5, 500 mg, 1.26 mmol) in dioxane (1 mL) was added 4M HCl/dioxane (5.0 mL) and the reaction was stirred at 25° C. for 2 h. The mixture was evaporated under reduced pressure to give N-(4-amino-5-ethoxypyridin-2-yl)acetamide hydrochloride (440 mg, crude) as a light yellow solid. LCMS m/z=196.0 [M+H]⁺.

### Preparation 63

### N-(4-amino-5-(2-methoxyethoxy)pyridin-2-yl)acetamide hydrochloride

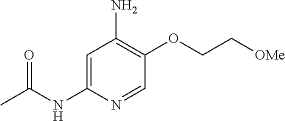 [SMILES: COCCOc1cnc(NC(C)=O)cc1N]

To a solution of tert-butyl (2-acetamido-5-(2-methoxyethoxy)pyridin-4-yl)carbamate (Preparation 32, 1.0 g, 3.07 mmol) in DCM (10 mL) was added 4M HCl/dioxane (10 mL) and the reaction was stirred at 25° C. for 1 h. The mixture was evaporated under reduced pressure to give N-(4-amino-5-(2-methoxyethoxy)pyridin-2-yl)acetamide hydrochloride (810 mg, crude) as a brown solid. LCMS m/z=226.1 [M+H]⁺. ¹H NMR: (500 MHz, DMSO-d₆) δ ppm: 12.94 (br s, 1H), 11.68 (s, 1H), 7.57 (s, 1H), 6.60 (s, 1H), 4.15-4.13 (m, 2H), 3.70-3.68 (m, 2H), 3.31 (s, 3H), 2.17 (s, 3H).

### Preparation 64

### N-(4-amino-5-(cyclopropylmethoxy)pyridin-2-yl)acetamide hydrochloride

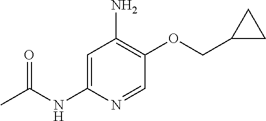 [SMILES: CC(=O)Nc1cc(N)c(OCC2CC2)cn1]

To a solution of tert-butyl (2-acetamido-5-(cyclopropylmethoxy)pyridin-4-yl)carbamate (Preparation 33, 110 mg, 0.342 mmol) in DCM (2 mL) was added 4M HCl/dioxane (5 mL) and the reaction was stirred at 25° C. for 3 h. The mixture was evaporated under reduced pressure to give N-(4-amino-5-(cyclopropylmethoxy)pyridin-2-yl)acetamide hydrochloride (70.0 mg, crude) as a white solid. LCMS m/z=222.1 [M+H]⁺.

### Preparations 65 to 81

The compounds in the following table were prepared from the appropriate Boc protected amine, following a similar procedure to that described in Preparation 64.

| Preparation | |
|---|---|
| No | Name, structure, starting material (SM), Data |
| 65 | N-(4-amino-5-(difluoromethoxy)pyridin-2-yl) |
| | acetamide hydrochloride |
| | 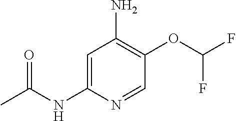 |
| | SM: tert-butyl (2-acetamido-5-(difluoromethoxy) |
| | pyridin-4-yl)carbamate |
| | (Preparation 34) |
| | 80 mg, crude as a yellow solid. |
| 66 | N-(4-amino-5-isopropoxypyridin-2- |
| | yl)acetamide hydrochloride |
| | 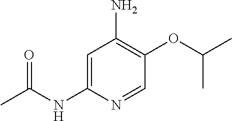 |
| | SM: tert-butyl (2-acetamido-5-isopropoxypyridin- |
| | 4-yl)carbamate |
| | (Preparation 41) |
| | crude, 520 mg. 1H NMR: (500 MHz, DMSO-d₆) δ ppm: |
| | 12.94 (s, 1H), 8.26 (s, 1H), 7.60 (s, 1H), 7.19 (br s, |
| | 1H), 6.72 (d, J = 3.0 Hz, 1H), 4.53-4.48 (m, |
| | 1H), 2.17 (s, 3H), 1.32-1.28 (m, 6H) |
| 67 | N-(4-amino-5-cyclopropoxypyridin-2-yl)acetamide |
| | hydrochloride |
| | 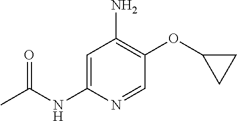 |
| | SM: tert-butyl (2-acetamido-5-cyclopropoxypyridin- |
| | 4-yl)carbamate |
| | (Preparation 42) |
| | white solid, 110 mg, crude. LCMS m/z = 208.3 |
| | [M + H]⁺ |
| 68 | N-(4-amino-5-cyclobutoxypyridin-2-yl)acetamide |
| | hydrochloride |
| | 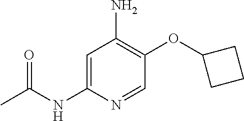 [SMILES: CC(=O)Nc1cc(N)c(OC2CCC2)cn1] |
| | SM: tert-butyl (2-acetamido-5-cyclobutoxypyridin- |
| | 4-yl)carbamate |
| | (Preparation 37) |
| | 60 mg, crude as a white solid. LCMS m/z = 222.1 |
| | [M + H]⁺ |
| 69 | N-(4-amino-5-(2,2,2-trifluoroethoxy)pyridin-2-yl) |
| | acetamide hydrochloride |
| | 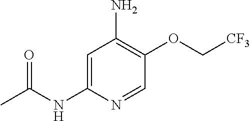 [SMILES: CC(=O)Nc1cc(N)c(OCC(F)(F)F)cn1] |
| | SM: tert-butyl (2-acetamido-5-(2,2,2-trifluoroethoxy) |
| | pyridin-4-yl)carbamate (Preparation 43) |
| | white solid, 152 mg, crude. LCMS m/z = 250.1 |
| | [M + H]⁺ |
| 70 | N-(4-amino-5-(2,2-difluoroethoxy)pyridin-2-yl) |
| | acetamide hydrochloride |
| | 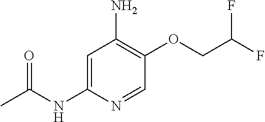 [SMILES: CC(=O)Nc1cc(N)c(OCC(F)F)cn1] |
| | SM: tert-butyl (2-acetamido-5-(2,2-difluoroethoxy) |
| | pyridin-4-yl)carbamate |
| | (Preparation 39) |
| | 279 mg, crude as a yellow solid. 1H NMR (400 MHz, |
| | MeOH-d₄) δ ppm: |
| | 7.58 (s, 1H), 6.41-6.12 (m, 2H), 4.40-4.33 (m, 2H), 2.22 |
| | (s, 3H). |
| 71 | N-(4-amino-5-(2-(dimethylamino)ethoxy)pyridin-2-yl) |
| | acetamide hydrochloride |
| | 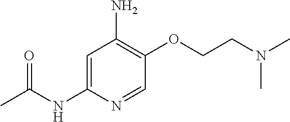 [SMILES: CC(=O)Nc1cc(N)c(OCCN(C)C)cn1] |
| | SM: tert-butyl (2-acetamido-5-(2-(dimethylamino) |
| | ethoxy)pyridin-4-yl)carbamate (Preparation 36) |
| | 135 mg, crude as a white solid |
| 72 | N-(4-amino-5-((3-methoxycyclobutyl)methoxy) |
| | pyridin-2-yl)acetamidehydrochloride |
| | 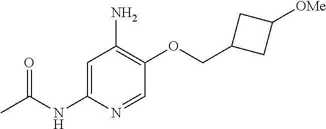 [SMILES: COC1CC(COc2cnc(NC(C)=O)cc2N)C1] |
| | SM:tert-butyl (2-acetamido-5-((3-methoxycyclobutyl) |
| | methoxy)pyridin-4-yl)carbamate (Preparation 44) |
| | 85 mg, crude as a yellow solid |
| 73 | N-(4-amino-5-ethoxypyridin-2-yl)propionamide |
| | hydrochloride |
| | 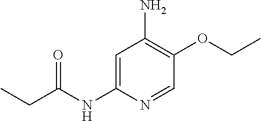 |
| | SM: tert-butyl (5-ethoxy-2-propionamidopyridin-4-yl) |
| | carbamate |
| | (Preparation 45) |
| | 170 mg, crude as a white solid, LCMS m/z = |
| | 210.2 [M + H]⁺ |
| 74 | N-(4-amino-5-ethoxypyridin-2-yl)-2- |
| | methoxyacetamide hydrochloride |
| | 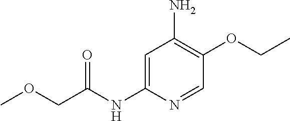 [SMILES: CCOc1cnc(NC(=O)COC)cc1N] |
| | SM: tert-butyl (5-ethoxy-2-(2-methoxyacetamido) |
| | pyridin-4-yl)carbamate |
| | (Preparation 46) |
| | 140 mg as a yellow solid. LCMS m/z = 226.2 [M + H]⁺ |
| 75 | N-(4-amino-5-ethoxypyridin-2-yl)-3- |
| | methoxypropanamide hydrochloride |
| | 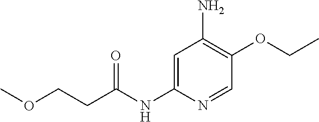 [SMILES: CCOc1cnc(NC(=O)CCOC)cc1N] |
| | SM: tert-butyl (5-ethoxy-2-(3-methoxypropanamido) |
| | pyridin-4-yl)carbamate |
| | (Preparation 47) |
| | 250 mg as a white solid. ¹H NMR: (500 MHz, |
| | MeOH-d₄) δ ppm: 7.45 (s, 1H), 6.42 (s, 1H) 4.17-4.10 |
| | (m, 2H), 3.73 (t, J = 6.0 Hz, 2H), 3.36 (s, 3H), 2.71- |
| | 2.67 (m, 2H) 1.48 (t, J = 7.0 Hz, 3H). |
| 76 | N-(4-amino-5-ethoxypyridin-2-yl)cyclopropanecarboxamide |
| | hydrochloride |
| | 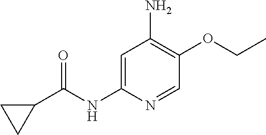 [SMILES: CCOc1cnc(NC(=O)C2CC2)cc1N] |
| | SM: tert-butyl (2-(cyclopropanecarboxamido)-5- |
| | ethoxypyridin-4-yl)carbamate |
| | (Preparation 48) |
| | 150 mg, crude as a yellow solid. LCMS m/z = |
| | 222.1 [M + H]⁺ |
| 77 | N-(4-amino-5-methylpyridin-2-yl)acetamide hydrochloride |
| | 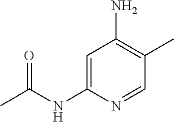 |
| | SM: tert-butyl (2-acetamido-5-methylpyridin-4-yl)carbamate |
| | (Preparation 52) |
| | 75 mg, crude as a white solid. LCMS m/z = 166.2 [M + H]⁺ |
| 78 | N-(4-amino-5-ethylpyridin-2-yl)acetamide hydrochloride |
| | 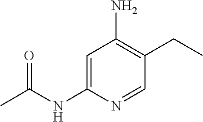 |
| | SM: tert-butyl (2-acetamido-5-ethylpyridin-4-yl)carbamate |
| | (Preparation 57) |
| | 148.5 mg, crude as a yellow solid |
| 79 | N-(4-amino-5-propylpyridin-2-yl)acetamide hydrochloride |
| | 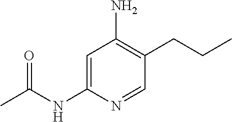 |
| | SM: tert-butyl (2-acetamido-5-propylpyridin-4-yl)carbamate |
| | (Preparation 59) |
| | 180.3 mg, crude as a yellow solid |
| 80 | N-(4-amino-5-isopropylpyridin-2-yl)acetamide |
| | hydrochloride |
| | 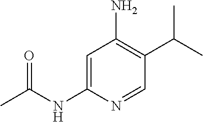 |
| | SM: tert-butyl (2-acetamido-5-isopropylpyridin-4- |
| | yl)carbamate (Preparation 58) |
| | 90 mg, crude as a white solid. 1H NMR: (500 MHz, DMSO- |
| | d₆) δ ppm: |
| | 12.95 (s, 1H), 11.79 (s, 1H), 8.28 (br s, 1H), 7.67 |
| | (s, 1H), 6.60 (s, 1H), 2.89- |
| | 2.85 (m, 1H), 2.18 (s, 3H), 1.14 (d, J = 6.5 Hz, 6H) |
| 81 | N-(4-amino-5-cyclopropylpyridin-2-yl)acetamide |
| | hydrochloride |
| | 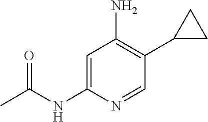 |
| | SM: tert-butyl (2-acetamido-5-cyclopropylpyridin- |
| | 4-yl)carbamate |
| | (Preparation 60) |
| | 90 mg, crude as a white solid. LCMS m/z = 192.1 [M + H]⁺ |

### Preparation 82

### N-(4-amino-5-(benzyloxy)pyridin-2-yl)acetamide hydrochloride

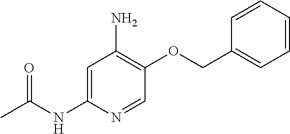 [SMILES: CC(=O)Nc1cc(N)c(OCc2ccccc2)cn1]

To a solution of tert-butyl (2-acetamido-5-(benzyloxy)pyridin-4-yl)carbamate (Preparation 38, 19 g, 53.16 mmol) in dioxane (200 mL) was added 4M HCl/Dioxane (200 mL) and the reaction was stirred at 20° C. for 1 h. The mixture was filtered and the filter cake was dried under reduced pressure followed by lyophilization to yield N-(4-amino-5-(benzyloxy)pyridin-2-yl)acetamide hydrochloride (14.93 g, 95.4% yield) as a white solid. LCMS m/z=258.0 [M+H]⁺

### Preparation 83N-(4-amino-5-(ethoxy-d₅)pyridin-2-yl)acetamide hydrochloride

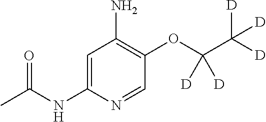 [SMILES: [2H]C([2H])([2H])C([2H])([2H])Oc1cnc(NC(C)=O)cc1N]

To a solution of tert-butyl (2-acetamido-5-(ethoxy-d₅)pyridin-4-yl)carbamate (Preparation 40, 1.26 g, 4.19 mmol) in dioxane (10 mL) was added HCl (4 M, 5.24 mL) and the reaction mixture was stirred at 40° C. for 3 h. The reaction was diluted with dioxane (10 mL), filtered through filter paper to collect the white solids, rinsing with an additional dioxane (10 mL). The solids were dissolved in MeOH (50 mL) and concentrated to dryness, then azeotroped with dioxane, to give N-(4-amino-5-(ethoxy-d₅)pyridin-2-yl)acetamide hydrochloride as a white solid, (1.06 g, crude). LCMS m/z=201.0 [M+H]⁺.

### Preparation 84

### 2-(1,1-difluoroethyl)-4-((1r,3r)-3-methoxycyclobutoxy)pyridine

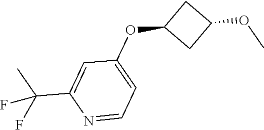 [SMILES: CO[C@H]1C[C@H](Oc2ccnc(C(C)(F)F)c2)C1]

To a solution of 4-chloro-2-(1,1-difluoroethyl)pyridine (5.84 g, 32.9 mmol) and (1r,3r)-3-methoxycyclobutan-1-ol (2.8 g, 27.42 mmol) in DMF (50 mL) was added NaH (1.32 g, 32.9 mmol, 60% purity). The reaction mixture was stirred at 60° C. for 16 h, quenched with water (50 mL) and extracted with EtOAc (3×50 mL). The combined organic layer was washed with brine (3×100 mL), dried over Na₂SO₄ and filtered. The filtrate was concentrated in vacuo and the residue was purified by column chromatography on silica gel (PE/EtOAc=10/1-5/1) to give 2-(1,1-difluoroethyl)-4-((1r,3r)-3-methoxycyclobutoxy)pyridine (5 g, 75% yield) as a colorless oil. 1H NMR (400 MHz, CDCl₃) δ ppm: 8.43 (d, J=5.6 Hz, 1H), 7.04 (d, J=1.8 Hz, 6.4 Hz, 1H), 6.73 (dd, J=2.4, 5.6 Hz, 1H), 4.94-4.88 (m, 1H), 4.20-4.09 (m, 1H), 3.28 (s, 3H), 2.54-2.38 (m, 4H), 1.99 (t, J=18.8 Hz, 3H).

### Preparation 85

### 2-(1,1-difluoroethyl)-4-((1s,3s)-3-methoxycyclobutoxy)pyridine

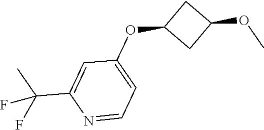 [SMILES: CO[C@H]1C[C@@H](Oc2ccnc(C(C)(F)F)c2)C1]

2-(1,1-Difluoroethyl)-4-((1s,3s)-3-methoxycyclobutoxy)pyridine was obtained as a colorless oil, 3.7 g, 91.4% yield, from 4-chloro-2-(1,1-difluoroethyl)pyridine and (1s,3s)-3-methoxycyclobutan-1-ol following the procedure described in Preparation 84. 1H NMR (400 MHz, CDCl₃) ppm: 8.43 (d, J=5.6 Hz, 1H), 7.06 (d, J=2.4 Hz, 1H), 6.76 (dd, J=2.4, 5.6 Hz, 1H), 4.44-4.37 (m, 1H), 3.73-3.66 (m, 1H), 3.28 (s, 3H), 2.96-2.59 (m, 2H), 2.17-2.14 (m, 2H), 1.99 (t, J=22.4 Hz, 3H).

### Preparation 86

### 2-(1,1-difluoroethyl)-4-((1r,3r)-3-methoxycyclobutoxy)pyridine 1-oxide

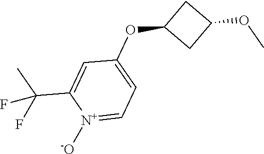

To a solution of 2-(1,1-difluoroethyl)-4-((1r,3r)-3-methoxycyclobutoxy)pyridine (Preparation 84, 7 g, 28.78 mmol) in DCM (100 mL) was added m-CPBA (7.31 g, 37.41 mmol, 80% purity) and the reaction was stirred at 25° C. for 16 h. The reaction mixture was quenched with sat.Na₂S₂O₃ (280 mL) and extracted with EtOAc (3×100 mL). The combined organic layer was dried over Na₂SO₄, filtered and the filtrate was concentrated in vacuo. The residue was purified by column chromatography on silica gel (PE/EtOAc=1/1, DCM/MeOH=10/1) to give 2-(1,1-difluoroethyl)-4-((1r,3r)-3-methoxycyclobutoxy)pyridine 1-oxide (7 g, 93.8% yield) as a colorless oil. 1H NMR: (400 MHz, CDCl₃) δ ppm 8.14 (d, J=7.2 Hz, 1H), 7.09 (d, J=7.2 Hz, 1H), 6.65 (dd, J=2.4, 7.2 Hz, 1H), 4.94-4.79 (m, 1H), 4.16-4.08 (m, 1H), 3.28 (s, 3H), 2.53-2.38 (m, 4H), 2.25 (t, J=19.2 Hz, 3H).

### Preparation 87

### 2-(1,1-difluoroethyl)-4-((1s,3s)-3-methoxycyclobutoxy)pyridine 1-oxide

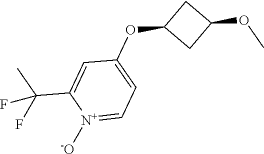

2-(1,1-Difluoroethyl)-4-((1s,3s)-3-methoxycyclobutoxy)pyridine 1-oxide was obtained as a colorless oil, 3.5 g, 88.8% yield, from 2-(1,1-difluoroethyl)-4-((1s,3s)-3-methoxycyclobutoxy)pyridine (Preparation 85) and m-CPBA, following the procedure described in Preparation 86. ¹H NMR (400 MHz, CDCl₃) δ ppm 8.19 (d, J=7.2 Hz, 1H), 7.08 (d, J=7.2 Hz, 1H), 6.80 (dd, J=3.4, 7.2 Hz, 1H), 4.41-4.34 (m, 1H), 3.73-3.70 (m, 1H), 3.30 (s, 3H), 2.97-2.90 (m, 2H), 2.25 (t, J=22.4 Hz, 3H), 2.20-2.16 (m, 2H).

### Preparation 88

### 2-chloro-6-(1,1-difluoroethyl)-4-((1r,3r)-3-methoxycyclobutoxy)pyridine

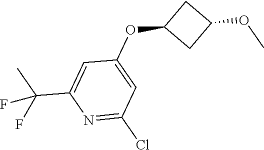 [SMILES: CO[C@H]1C[C@H](Oc2cc(Cl)nc(C(C)(F)F)c2)C1]

To a solution of 2-(1,1-difluoroethyl)-4-((1r,3r)-3-methoxycyclobutoxy)pyridine 1-oxide (Preparation 86, 7 g, 27 mmol) was added POCl₃ (63.6 mL, 682.72 mmol) and the reaction mixture was stirred at 100° C. for 16 h. The mixture was concentrated and dissolved in DCM (20 mL). The solution was poured into water (50 mL) and quenched with sat.NaHCO₃ (40 mL) until pH>8. The aqueous layer was extracted with DCM (80 mL×3), the combined organic layers were washed with brine (100 mL), dried over Na₂SO₄ and concentrated in vacuo. The crude product was purified by column chromatography on silica gel (PE/EtOAc=10/1) to give 2-chloro-6-(1,1-difluoroethyl)-4-((1r,3r)-3-methoxycyclobutoxy)pyridine (3.8 g, 50.7% yield) as a yellow oil. LCMS m/z=278.1 [M+H]⁺

### Preparation 89

### 2-chloro-6-(1,1-difluoroethyl)-4-((1s,3s)-3-methoxycyclobutoxy)pyridine

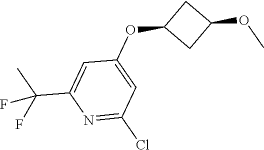 [SMILES: CO[C@H]1C[C@@H](Oc2cc(Cl)nc(C(C)(F)F)c2)C1]

2-Chloro-6-(1,1-difluoroethyl)-4-((1s,3s)-3-methoxycyclobutoxy)pyridine was obtained as a yellow oil, from 2-(1,1-difluoroethyl)-4-((1s,3s)-3-methoxycyclobutoxy)pyridine 1-oxide (Preparation 87), following the procedure described in Preparation 88. LCMS m/z=278.1 [M+H]⁺

### Preparation 90

### 2-(1,1-difluoroethyl)pyrimidine-4,6-diol

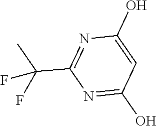

To a solution of malonamide (15.0 g, 146.9 mmol) in EtOH (500 mL) was added t-BuONa (49.4 g, 514.24 mmol) and the solution was stirred at 25° C. for 30 mins. Ethyl 2,2-difluoropropanoate (50.7 g, 367.32 mmol) was added and the reaction stirred under reflux at 100° C. for 16 h. The reaction was cooled, 4N HCl (75 mL) was added and the mixture concentrated. The resulting solid was collected and concentrated to give 2-(1,1-difluoroethyl)pyrimidine-4,6-diol (20.0 g, 77.3% yield) as a yellow solid. LCMS m/z=177.0 [M+H]⁺

### Preparation 91

### 4,6-dichloro-2-(1,1-difluoroethyl)pyrimidine

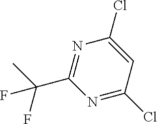

A solution of 2-(1,1-difluoroethyl)pyrimidine-4,6-diol (Preparation 90, 20.0 g, 113.56 mmol) in POCl₃ (200 mL, 2.15 mol) was stirred at 100° C. for 8 h. The mixture was evaporated to dryness, the residue diluted with DCM (150 mL) and added slowly into water. The mixture was extracted with DCM (200 mL×3), the combined organic phase was washed with brine (300 mL), dried over anhydrous Na₂SO₄, filtered and concentrated. The crude was purified by column chromatography (EtOAc in PE 0% to 10%) to give 4,6-dichloro-2-(1,1-difluoroethyl)pyrimidine (23.0 g, 95.1% yield) as a yellow liquid. LCMS m/z=213.0 [M+H]⁺

### Preparation 92

### 4-chloro-2-(1,1-difluoroethyl)-6-methylpyrimidine

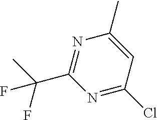

To a solution of 4,6-dichloro-2-(1,1-difluoroethyl)pyrimidine (Preparation 91, 75.0 g, 352.1 mmol) in THF (750 mL) and NMP (75 mL) at 23° C. was added Fe(acac)₃ (12.44 g, 35.21 mmol). The mixture was purged with N₂, then cooled to −20° C. To the cooled solution, CH₃MgBr (3 M, 117.4 mL) was added dropwise, maintaining the internal temperature at between −20 and −10° C. during addition. The resulting mixture was stirred at −20° C. for 2 h. The mixture was poured into sat. aq. NH₄Cl (1.5 L) and extracted with MTBE (1.0 L). The organic layer was washed with brine (100 mL), dried over anhydrous MgSO₄, filtered and concentrated. The resulting reddish-brown liquid was purified by Prep-HPLC (Method F, Gradient: 40%-58%). The desired fractions were evaporated in vacuo to remove MeCN. The mixture was extracted with MTBE (500 mL), the organic phase was washed with brine (100 mL), dried over anhydrous MgSO₄, filtered and concentrated to afford 4-chloro-2-(1,1-difluoroethyl)-6-methylpyrimidine as a yellow liquid. LCMS m/z=193.1 [M+H]⁺.

### Preparation 93

### 2-(1,1-difluoroethyl)-6-methylpyrimidin-4-amine

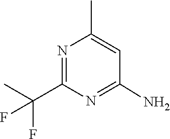

To a stirring solution of 4-chloro-2-(1,1-difluoroethyl)-6-methylpyrimidine (Preparation 92, 200 mg, 1.04 mmol) in anhydrous dioxane (2 mL) were added Pd₂(dba)₃ (47.55 mg, 0.0519 mmol), Xantphos (60.09 mg, 0.104 mmol) and Cs₂CO₃ (676.70 mg, 2.08 mmol). Benzophenone imine (225.84 mg, 1.25 mmol) was then added and the reaction mixture was heated to 90° C. for 1 h. The reaction was diluted with EtOAc, washed with water and dried over Na₂SO₄ and concentrated in vacuo. The residue was dissolved in MeOH (15 mL), NaOAc (196.87 mg, 2.40 mmol) and hydroxylamine hydrochloride (254.33 mg, 3.66 mmol) were added and the reaction stirred for 30 min at rt. The solvent was evaporated and the residue was redissolved in THF. The THF solution was washed once with a solution of 1 N NaOH-brine (1:3 v/v), dried over MgSO₄ and evaporated under reduced pressure. The residue was purified by silica gel chromatography using 0-100% heptane/EtOAc-EtOH (3:1) to afford 2-(1,1-difluoroethyl)-6-methylpyrimidin-4-amine (80 mg, 44.5% yield). 1H NMR (500 MHz, MeOH-d₄) δ 6.35 (s, 1H), 2.29 (s, 3H), 1.87 (t, J=18.6 Hz, 3H).

### Preparation 94

### 4-chloro-2-(1,1-difluoroethyl)-6-ethylpyrimidine

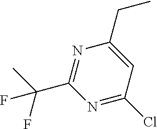

To a solution of 4,6-dichloro-2-(1,1-difluoroethyl)pyrimidine (Preparation 91, 1.5 g, 7.04 mmol) in water (3 mL) and dioxane (15 mL) was added ethylboronic acid (520 mg, 7.04 mmol), K₂CO₃ (1.46 g, 10.6 mmol) and Pd(dppf)Cl₂ (515 mg, 0.70 mmol) and the mixture was stirred at 100° C. for 16 h under N₂. The mixture was diluted with water (10 mL) and extracted with EtOAc (25 mL×3). The combined organic layers were washed with brine (25 mL), dried over Na₂SO₄, filtered and concentrated. The residue was purified by chromatography on silica gel (PE/EtOAc 1/0 to 10/1) to give 4-chloro-2-(1,1-difluoroethyl)-6-ethylpyrimidine (410 mg, 28.2% yield) as a yellow solid. ¹H NMR: (400 MHz, CDCl₃) δ ppm: 7.28 (s, 1H), 2.86 (q, J=7.5 Hz, 2H), 2.06 (t, J=18.5 Hz 3H), 1.34 (t, J=7.5 Hz, 3H).

### Preparation 95

### 4-chloro-2-(1,1-difluoroethyl)-6-(prop-1-en-2-yl)pyrimidine

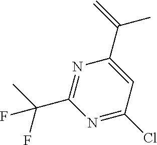

To a mixture of 4,6-dichloro-2-(1,1-difluoroethyl)pyrimidine (Preparation 91, 320 mg, 1.50 mmol), 4,4,5,5-tetramethyl-2-(prop-1-en-2-yl)-1,3,2-dioxaborolane (252 mg, 1.50 mmol) and K₂CO₃ (415 mg, 3.00 mmol) in dioxane (10 mL) and water (2 mL) was added Pd(dppf)Cl₂ (110 mg, 0.150 mmol) and the reaction was stirred at 70° C. for 1 h. The reaction mixture was concentrated to dryness and the residue purified by chromatography on silica gel (PE/EtOAc 0/1 to 3/1) to give 4-chloro-2-(1,1-difluoroethyl)-6-(prop-1-en-2-yl)pyrimidine (220 mg, 67.0% yield) as yellow oil. LCMS m/z=219.1 [M+H]⁺.

### Preparation 96

### 4-chloro-6-cyclopropyl-2-(1,1-difluoroethyl)pyrimidine

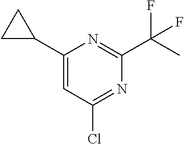

To a solution of 4,6-dichloro-2-(1,1-difluoroethyl)pyrimidine (Preparation 91, 1.0 g, 4.69 mmol) in dioxane (10.0 mL) and water (1.0 mL) was added cyclopropylboronic acid (483.9 mg, 5.63 mmol), K₂CO₃ (1.3 g, 9.39 mmol) and Pd (dppf)Cl₂ (343.5 mg, 469.5 umol). The mixture was stirred at 70° C. for 2 h under N₂. The mixture was diluted with water (15 mL), extracted with EtOAc (20 mL×3), the combined organic phase was washed with brine (60 mL), dried over anhydrous Na₂SO₄, filtered and concentrated. The crude was purified by column chromatography (PE/EtOAc=1/0 to 5/1) to give 4-chloro-6-cyclopropyl-2-(1,1-difluoroethyl)pyrimidine (805.6 mg, 78.5% yield) as colorless oil. LCMS m/z=219.1 [M+H]⁺

### Preparation 97

### 4-chloro-2-(1,1-difluoroethyl)-6-methoxypyrimidine

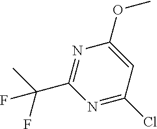

To a solution of 4,6-dichloro-2-(1,1-difluoroethyl)pyrimidine (Preparation 91, 300 mg, 1.41 mmol) in MeOH (5.0 mL) was added NaOMe (76.1 mg, 1.41 mmol) at 0° C. and the reaction was stirred at 25° C. for 2 h. The mixture was concentrated under reduced pressure and the residue was purified by chromatography on silica gel (PE/EtOAc 15/1 to 5/1) to give 4-chloro-2-(1,1-difluoroethyl)-6-methoxypyrimidine (260 mg, 88.5% yield) as colourless oil. ¹H NMR: (400 MHz, CDCl₃) δ ppm: 6.81 (s, 1H), 4.08 (s, 3H), 2.02 (t, J=18.5 Hz, 3H).

### Preparation 98

### 2,4-dichloro-5-fluoro-6-methylpyrimidine

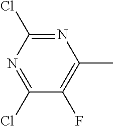

To a solution of 2,4-dichloro-5-fluoropyrimidine (15.0 g, 89.84 mmol) in DME (150.0 mL) was slowly added MeMgBr (3 M, 44.9 mL) at 0° C. under N₂ and the mixture was stirred at 15° C. for 1 h. TEA (9.1 g, 89.84 mmol) in THF (10.0 mL) was added, the solution stirred at 0° C. for 5 mins, then I₂ (22.8 g, 89.84 mmol) in THF (16.0 mL) was slowly added and the reaction stirred at 15° C. for 2 h. The mixture was quenched with H₂O (200 mL) and the pH adjusted to 1 with 5N HCl. The mixture was extracted with EtOAc (150.0 mL×3), the combined organic phase was washed with brine (300 mL), dried over Na₂SO₄, filtered and concentrated in vacuo. The crude was purified by column chromatography on silica gel (PE/EtOAc=0/1 to 10/1) to give 2,4-dichloro-5-fluoro-6-methylpyrimidine (9.3 g, 57% yield) as yellow oil. ¹H NMR (500 MHz, CDCl₃) δ ppm: 2.56 (d, J=2.5 Hz, 3H).

### Preparation 99

### 4-(benzyloxy)-2-chloro-5-fluoropyrimidine

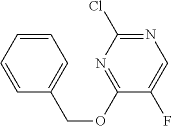 [SMILES: Fc1cnc(Cl)nc1OCc1ccccc1]

A solution of BnOH (6.5 g, 59.89 mmol) and t-BuONa (5.8 g, 59.9 mmol) in toluene (150.0 mL) was stirred at 0° C. for 10 mins. 2,4-Dichloro-5-fluoropyrimidine (10.0 g, 59.9 mmol) was added slowly and the resulting mixture was stirred at 20° C. for 1 h. The mixture was poured into H₂O (100 mL), extracted with EtOAc (3×100 mL), the combined organic layer was dried over anhydrous Na₂SO₄, filtered and concentrated in vacuo. The residue was purified by chromatography on silica gel (PE/EtOAc=20/1 to 5/1) to give 4-(benzyloxy)-2-chloro-5-fluoropyrimidine (12.5 g, 87.5% yield) as a white solid. ¹H NMR: (500 MHz, CDCl₃) δ ppm: 8.21 (s, 1H), 7.49-7.42 (m, 2H), 7.41-7.38 (m, 3H), 5.51 (s, 2H).

### Preparation 100

### 4-(benzyloxy)-2-chloro-5-fluoro-6-methylpyrimidine

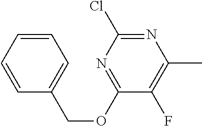 [SMILES: Cc1nc(Cl)nc(OCc2ccccc2)c1F]

4-(Benzyloxy)-2-chloro-5-fluoro-6-methylpyrimidine was obtained as a colorless oil, 7.9 g, 58.2% yield from 2,4-dichloro-5-fluoro-6-methylpyrimidine (Preparation 98) and BnOH, following the procedure described in Preparation 99. ¹H NMR: (500 MHz, CDCl₃) δ ppm: 7.48-7.46 (m, 2H), 7.42-7.36 (m, 3H), 5.48 (s, 2H), 2.44 (d, J=3.0 Hz, 3H).

### Preparation 101

### methyl 4-(benzyloxy)-5-fluoropyrimidine-2-carboxylate

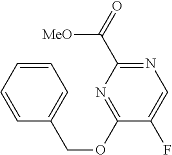 [SMILES: COC(=O)c1ncc(F)c(OCc2ccccc2)n1]

To a solution of 4-(benzyloxy)-2-chloro-5-fluoropyrimidine (Preparation 99, 26.0 g, 109 mmol) in MeOH (300 mL) was added Pd(dppf)Cl₂ (1.6 g, 2.18 mmol) and TEA (22.1 g, 217.9 mmol). The resulting mixture was stirred at 80° C. for 16 h under CO (50 psi). The mixture was concentrated in vacuo and the residue was purified by chromatography on silica gel (PE/EtOAc=20/1 to 3/1) to give methyl 4-(benzyloxy)-5-fluoropyrimidine-2-carboxylate (12.0 g, 42% yield) as a white solid. ¹H NMR: (400 MHz, CDCl₃) δ ppm: 8.44 (d, J=2.0 Hz, 1H), 7.53-7.39 (m, 2H), 7.38-7.35 (m, 3H), 5.61 (s, 2H), 4.03 (s, 3H).

### Preparation 102

### methyl 4-(benzyloxy)-5-fluoro-6-methylpyrimidine-2-carboxylate

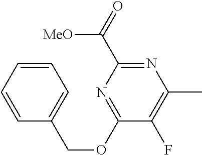 [SMILES: COC(=O)c1nc(C)c(F)c(OCc2ccccc2)n1]

Methyl 4-(benzyloxy)-5-fluoro-6-methylpyrimidine-2-carboxylate was obtained as a yellow oil, 3.1 g, 35.8% yield from 4-(benzyloxy)-2-chloro-5-fluoro-6-methylpyrimidine (Preparation 100) and MeOH, following the procedure described in Preparation 101. ¹H NMR: (400 MHz, CDCl₃) δ ppm: 7.53-7.51 (m, 2H), 7.40-7.35 (m, 3H), 5.58 (s, 2H), 4.03 (s, 3H), 2.55 (d, J=2.8 Hz, 3H).

### Preparation 103

### 1-(4-(benzyloxy)-5-fluoropyrimidin-2-yl)ethan-1-one

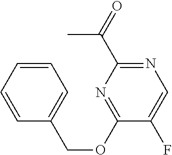 [SMILES: CC(=O)c1ncc(F)c(OCc2ccccc2)n1]

To a solution of methyl 4-(benzyloxy)-5-fluoropyrimidine-2-carboxylate (Preparation 101, 12.0 g, 45.76 mmol) in THF (120 mL) was added dropwise MeMgBr (3 M, 18.30 mL) at −78° C. and the solution was stirred for 2 h under N₂. The mixture was quenched with NH₄Cl (sat. 30 mL) at −78° C., poured into H₂O (60 mL) and extracted with EtOAc (3×100 mL). The combined organic layer was dried over anhydrous Na₂SO₄, filtered and concentrated in vacuo. The residue was purified by chromatography on silica gel (PE/EtOAc=20/1 to 3/1) to give 1-(4-(benzyloxy)-5-fluoropyrimidin-2-yl)ethan-1-one (7.3 g, 64.8% yield) as a white solid. ¹H NMR: (400 MHz, CDCl₃) δ ppm: 8.45 (d, J=2.0 Hz, 1H), 7.52-7.49 (m, 2H), 7.42-7.37 (m, 3H), 5.60 (s, 2H), 2.73 (s, 3H).

### Preparation 104

### 1-(4-(benzyloxy)-5-fluoro-6-methylpyrimidin-2-yl)ethan-1-one

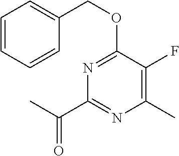 [SMILES: CC(=O)c1nc(C)c(F)c(OCc2ccccc2)n1]

1-(4-(Benzyloxy)-5-fluoro-6-methylpyrimidin-2-yl)ethan-1-one was obtained as a yellow oil, 1.6 g, 54.8% yield, from methyl 4-(benzyloxy)-5-fluoro-6-methylpyrimidine-2-carboxylate (Preparation 102, and MeMgBr following the procedure described in Preparation 103. ¹H NMR (400 MHz, CDCl₃) δ ppm: 7.51-7.49 (m, 2H), 7.40-7.36 (m, 3H), 5.57 (s, 2H), 2.71 (s, 3H), 2.54 (d, J=3.2 Hz, 3H).

### Preparation 105

### 4-(benzyloxy)-2-(1,1-difluoroethyl)-5-fluoropyrimidine

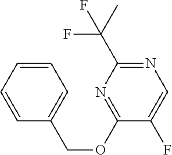 [SMILES: CC(F)(F)c1ncc(F)c(OCc2ccccc2)n1]

To a solution of 1-(4-(benzyloxy)-5-fluoropyrimidin-2-yl)ethan-1-one (Preparation 103, 7.3 g, 29.65 mmol) in DCM (80.0 mL) was added DAST (23.9 g, 148.23 mmol). The resulting mixture was stirred at 20° C. for 12 h. The mixture was slowly poured into H₂O (80 mL), extracted with DCM (3×80 mL), the combined organic extracts were washed with brine (2×80 mL), dried over Na₂SO₄ and filtered. The filtrate was concentrated under reduced pressure and the residue was purified by chromatography on silica gel (PE/EtOAc=20/1 to 5/1) to give 4-(benzyloxy)-2-(1,1-difluoroethyl)-5-fluoropyrimidine (6.7 g, 84% yield) as colorless oil. ¹H NMR: (400 MHz, CDCl₃) δ ppm: 8.38 (d, J=2.4 Hz, 1H), 7.51-7.40 (m, 2H), 7.39-7.36 (m, 3H), 5.56 (s, 2H), 2.02 (t, J=18.4 Hz, 3H).

### Preparation 106

### 4-(benzyloxy)-2-(1,1-difluoroethyl)-5-fluoro-6-methylpyrimidine

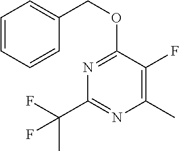 [SMILES: Cc1nc(C(C)(F)F)nc(OCc2ccccc2)c1F]

4-(Benzyloxy)-2-(1,1-difluoroethyl)-5-fluoro-6-methylpyrimidine was obtained, 1.6 g, 95.9% yield, as a yellow oil, from 1-(4-(benzyloxy)-5-fluoro-6-methylpyrimidin-2-yl)ethan-1-one (Preparation 104) following the procedure described in Preparation 105. ¹H NMR (500 MHz, CDCl₃) δ ppm: 7.48-7.46 (m, 2H), 7.38-7.32 (m, 3H), 5.51 (s, 2H), 2.48 (d, J=3.0 Hz, 3H), 1.98 (t, J=18.0 Hz, 3H).

### Preparation 107

### 2-(1,1-difluoroethyl)-5-fluoropyrimidin-4-ol

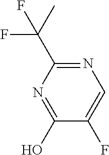

A solution of 4-(benzyloxy)-2-(1,1-difluoroethyl)-5-fluoropyrimidine (Preparation 105, 6.7 g, 24.9 mmol) in TFA (40 mL) was stirred at 100° C. for 12 h. The reaction was concentrated under reduced pressure and the residue was purified by chromatography on silica gel (PE/EtOAc=20/1 to 3/1) to give 2-(1,1-difluoroethyl)-5-fluoropyrimidin-4-ol (3.2 g, 72.1% yield) as a white solid. ¹H NMR: (400 MHz, CDCl₃) δ ppm: 11.61 (br s, 1H), 7.93 (d, J=2.4 Hz, 1H), 2.03 (t, J=18.8 Hz, 3H).

### Preparation 108

### 2-(1,1-difluoroethyl)-5-fluoro-6-methylpyrimidin-4-ol

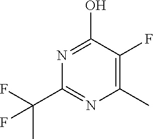

2-(1,1-Difluoroethyl)-5-fluoro-6-methylpyrimidin-4-ol was obtained as a colorless oil, 780 mg, 73.5%, from 4-(benzyloxy)-2-(1,1-difluoroethyl)-5-fluoro-6-methylpyrimidine (Preparation 106) following the procedure described in Preparation 107. ¹H NMR: (500 MHz, CDCl₃) δ ppm: 2.40 (d, J=3.5 Hz, 3H), 2.02 (t, J=18.5 Hz, 3H).

### Preparation 109

### 4-chloro-2-(1,1-difluoroethyl)-5-fluoropyrimidine

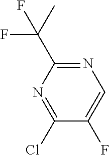

A solution of 2-(1,1-difluoroethyl)-5-fluoropyrimidin-4-ol (Preparation 107, 3.2 g, 18 mmol) in POCl₃ (20.0 mL) was stirred at 100° C. for 2 h. The mixture was slowly poured into ice/water (50 mL) and extracted with EtOAc (3×60 mL). The combined organic phase was washed with brine (2×50 mL), dried over Na₂SO₄ and filtered. The filtrate was concentrated under reduced pressure and the residue was purified by chromatography on silica gel (PE/EtOAc=20/1 to 5/1) to give 4-chloro-2-(1,1-difluoroethyl)-5-fluoropyrimidine (2.3 g, 65.1% yield) as colorless oil. LCMS m/z=197.1 [M+H]⁺

### Preparation 110

### 4-chloro-2-(1,1-difluoroethyl)-5-fluoro-6-methylpyrimidine

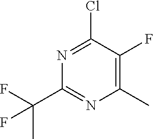

4-Chloro-2-(1,1-difluoroethyl)-5-fluoro-6-methylpyrimidine was obtained as a colorless oil, 3.1 g, 90.7% yield from 2-(1,1-difluoroethyl)-5-fluoro-6-methylpyrimidin-4-ol (Preparation 108) following the procedure described in Preparation 109. LCMS m/z=211 [M+H]⁺

### Preparation 111

### 4-chloro-2-(1,1-difluoroethyl)-6-((1r,3r)-3-methoxycyclobutoxy)pyrimidine

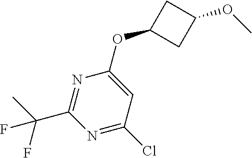 [SMILES: CO[C@H]1C[C@H](Oc2cc(Cl)nc(C(C)(F)F)n2)C1]

To a solution of 4,6-dichloro-2-(1,1-difluoroethyl)pyrimidine (Preparation 91, 400 mg, 1.88 mmol) and Cs₂CO₃ (1.9 g, 5.63 mmol) in DMF (6.0 mL) was added (1r,3r)-3-methoxycyclobutan-1-ol (211 mg, 2.07 mmol) and the reaction was stirred at 25° C. for 1 h. The mixture was diluted with water (80 mL) and extracted with EtOAc (40 mL×3). The combined organic phase was washed with brine (50 mL), dried over anhydrous Na₂SO₄, filtered and concentrated to give 4-chloro-2-(1,1-difluoroethyl)-6-((1r,3r)-3-methoxycyclobutoxy)pyrimidine (450 mg, 86.0% yield) as a yellow oil. LCMS m/z=278.9 [M+H]⁺. ¹H NMR: (400 MHz, CDCl₃) δ ppm: 6.77 (s, 1H), 5.47-5.41 (m, 1H), 4.15-4.08 (m, 1H), 3.27 (s, 3H), 2.57-2.49 (m, 2H), 2.43-2.40 (m, 2H), 1.99 (t, J=18.4 Hz, 3H).

### Preparation 112

### 4-chloro-2-(1,1-difluoroethyl)-6-(2-methoxyethoxy)pyrimidine

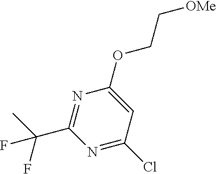 [SMILES: COCCOc1cc(Cl)nc(C(C)(F)F)n1]

To a solution of 4,6-dichloro-2-(1,1-difluoroethyl)pyrimidine (Preparation 91, 200 mg, 0.939 mmol) and 2-methoxyethan-1-ol (78.6 mg, 1.03 mmol) in DMF (2 mL) was added Cs₂CO₃ (918 mg, 2.82 mmol) and the reaction was stirred at 25° C. for 1 h. The mixture was concentrated and then water (10 mL) was added. The aqueous mixture was extracted with EtOAc (10 mL×3), the combined organic layers were washed with brine (10 mL×3), dried over Na₂SO₄, filtered and concentrated. The residue was purified by prep-TLC (PE/EtOAc 3/1) to afford 4-chloro-2-(1,1-difluoroethyl)-6-(2-methoxyethoxy)pyrimidine (150 mg, 63.2% yield) as a yellow oil. ¹H NMR: (500 MHz, CDCl₃) δ ppm: 6.86 (s, 1H), 4.62-4.60 (m, 2H), 3.75-3.74 (m, 2H), 3.43 (s, 3H), 2.05-1.97 (m, 3H).

### Preparation 113

### 3-(benzyloxy)-1-methylcyclobutan-1-ol

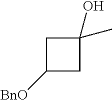

To a solution of 3-(benzyloxy)cyclobutan-1-one (5.0 g, 28.4 mmol) in THF (50 mL) was added CH₃MgCl (3 M, 10.4 mL) at −78° C. under N₂ and the reaction was stirred at 25° C. for 2 h. The mixture was diluted with water (60 mL) and extracted with EtOAc (100 mL×3). The combined organic phase was washed with brine (60 mL×3), dried over anhydrous Na₂SO₄, filtered and concentrated. The crude was purified by chromatography on silica gel (PE/EtOAc 1/0 to 1/1) to give 3-(benzyloxy)-1-methylcyclobutan-1-ol (4.9 g, 89.5% yield) as colorless oil. ¹H NMR: (400 MHz, CDCl₃) δ ppm: 7.35-7.28 (m, 5H), 4.42 (s, 2H), 3.76-3.69 (m, 1H), 2.47-2.42 (m, 2H), 2.12-2.09 (m, 2H), 1.31 (s, 3H).

### Preparation 114

### (3-(benzyloxy)-1-methylcyclobutoxy)(tert-butyl)dimethylsilane

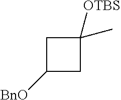 [SMILES: CC1(O[Si](C)(C)C(C)(C)C)CC(OCc2ccccc2)C1]

To a solution of 3-(benzyloxy)-1-methylcyclobutan-1-ol (Preparation 113, 2.0 g, 10.4 mmol) and imidazole (3.5 g, 52.0 mmol) in DCM (50 mL) was added TBSCI (2.8 g, 18.7 mmol) at 0° C. The mixture was stirred at 25° C. for 16 h then diluted with water (50 mL) and extracted with EtOAc (60 mL×3). The combined organic phase was washed with brine (60 mL), dried over anhydrous Na₂SO₄, filtered and concentrated. The crude was purified by chromatography on silica gel (PE/EtOAc 3/1) to give (3-(benzyloxy)-1-methylcyclobutoxy)(tert-butyl)dimethylsilane (2.5 g, 78.4% yield) as colorless oil. ¹H NMR: (500 MHz, CDCl₃) δ ppm: 7.34-7.28 (m, 5H), 4.40 (s, 2H), 3.69-3.63 (m, 1H), 2.40-2.36 (m, 2H), 2.16-2.14 (m, 2H), 1.28 (s, 3H), 0.87 (s, 9H), 0.07 (s, 6H).

### Preparation 115

### 3 ((tert-butyldimethylsilyl)oxy)-3-methylcyclobutan-1-ol

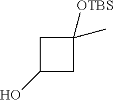

To a solution of (3-(benzyloxy)-1-methylcyclobutoxy)(tert-butyl)dimethylsilane (Preparation 114, 2.5 g, 8.16 mmol) in MeOH (200 mL) was added Pd/C (868 mg, 0.816 mmol, 10% purity) under H₂ (40 psi) and the reaction was stirred at 25° C. for 32 h. The mixture was filtered and concentrated to give 3-((tert-butyldimethylsilyl)oxy)-3-methylcyclobutan-1-ol (1.8 g, crude) as colorless oil. ¹H NMR: (500 MHz, CDCl₃) δ ppm: 3.94-3.90 (m, 1H), 2.48-2.44 (m, 2H), 2.08-2.05 (m, 2H), 1.28 (s, 3H), 0.88 (s, 9H), 0.07 (s, 6H).

### Preparation 116

### 4-(3-((tert-butyldimethylsilyl)oxy)-3-methylcyclobutoxy)-6-chloro-2-(1,1-difluoroethyl)pyrimidine

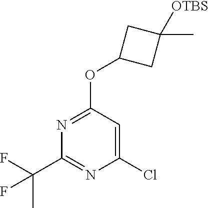 [SMILES: CC1(O[Si](C)(C)C(C)(C)C)CC(Oc2cc(Cl)nc(C(C)(F)F)n2)C1]

To a solution of 3-((tert-butyldimethylsilyl)oxy)-3-methylcyclobutan-1-ol (Preparation 115, 300 mg, 1.39 mmol) in THF (10 mL) was added NaH (83.2 mg, 2.08 mmol, 60% purity) at 0° C. The mixture was stirred at 25° C. for 30 mins followed by addition of 4,6-dichloro-2-(1,1-difluoroethyl)pyrimidine (Preparation 91, 354 mg, 1.66 mmol). The mixture was stirred at 25° C. for 1 h, then diluted with water (15 mL) and extracted with EtOAc (20 mL×3). The combined organic phase was washed with brine (60 mL), dried over anhydrous Na₂SO₄, filtered and concentrated. The crude was purified by chromatography on silica gel (PE/EtOAc 1/0 to 10/1) to give 4-(3-((tert-butyldimethylsilyl)oxy)-3-methylcyclobutoxy)-6-chloro-2-(1,1-difluoroethyl)pyrimidine (400 mg, 73.4% yield) as colorless oil. ¹H NMR: (400 MHz, CDCl₃) δ ppm: 6.77 (s, 1H), 4.94-4.86 (m, 1H), 2.67-2.62 (m, 2H), 2.31-2.28 (m, 2H), 2.00 (t, J=18.4 Hz, 1H), 1.40 (s, 3H), 0.88 (s, 9H), 0.08 (s, 6H).

### Preparation 117

### N-(5-bromo-2-chloropyridin-4-yl)-6-chloro-2-(1,1-difluoroethyl)pyrimidin-4-amine

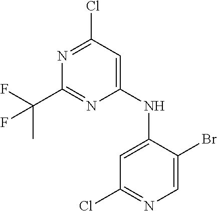 [SMILES: CC(F)(F)c1nc(Cl)cc(Nc2cc(Cl)ncc2Br)n1]

A mixture of 5-bromo-2-chloropyridin-4-amine (250 mg, 1.21 mmol), Cs₂CO₃ (1 g, 3.07 mmol) and DMF (5 mL) was stirred for 5 mins, then 4,6-dichloro-2-(1,1-difluoroethyl)pyrimidine (Preparation 91, 300 mg, 1.41 mmol) was added and the reaction stirred at 35° C. for 2 h. The reaction was concentrated to remove solvent, then diluted with water (10 mL) and extracted with EtOAc (3×10 mL). The combined organic layers were concentrated to dryness and the crude product purified by silica gel chromatography (heptane to 50% EtOAc) to give N-(5-bromo-2-chloropyridin-4-yl)-6-chloro-2-(1,1-difluoroethyl)pyrimidin-4-amine (415 mg, 89.7% yield). LCMS m/z=385.1 [M+H]⁺. 1H NMR (DMSO-d₆) δ: 9.91 (s, 1H), 8.60 (s, 1H), 8.33 (s, 1H), 7.55-7.44 (m, 1H), 1.98 (t, J=18.9 Hz, 3H).

### Preparation 118

### N-(5-bromo-2-chloropyridin-4-yl)-2-(1,1-difluoroethyl)-6-methoxypyrimidin-4-amine

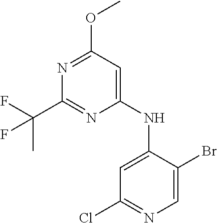 [SMILES: COc1cc(Nc2cc(Cl)ncc2Br)nc(C(C)(F)F)n1]

To a solution of N-(5-bromo-2-chloropyridin-4-yl)-6-chloro-2-(1,1-difluoroethyl)pyrimidin-4-amine (Preparation 117, 415 mg, 1.08 mmol) in MeOH (10 mL) was added K₂CO₃ (400 mg) and the reaction stirred at 70° C. for 20 h. The cooled reaction was concentrated to dryness, diluted with water (10 mL) and extracted with EtOAc (3×10 mL). The combined organic layers were concentrated to dryness and purified by silica gel chromatography (heptane to 50% EtOAc) to give N-(5-bromo-2-chloropyridin-4-yl)-2-(1,1-difluoroethyl)-6-methoxypyrimidin-4-amine (390 mg, 95.4% yield). LCMS m/z=381.1 [M+H]⁺

### Preparation 119

### N-(2-chloro-5-(1-methyl-1H-pyrazol-3-yl)pyridin-4-yl)-2-(1,1-difluoroethyl)-6-methoxypyrimidin-4-amine

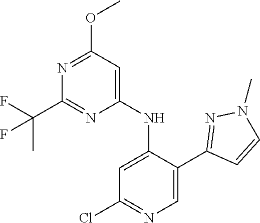 [SMILES: COc1cc(Nc2cc(Cl)ncc2-c2ccn(C)n2)nc(C(C)(F)F)n1]

To a vial was added N-(5-bromo-2-chloropyridin-4-yl)-2-(1,1-difluoroethyl)-6-methoxypyrimidin-4-amine (Preparation 118, 150 mg, 0.395 mmol), 1-methyl-3-(4,4,5,5-tetramethyl-1,3,2-dioxaborolan-2-yl)pyrazole (100 mg, 0.481 mmol), K₂CO₃ (150 mg, 1.09 mmol) and Pd(dppf)Cl₂ (15 mg, 0.0205 mmol) in dioxane (3 mL) and water (1 mL), the vial sealed and the reaction stirred at 80° C. for 20 h. The reaction was removed from heating, cooled to rt, diluted with water (10 mL) and extracted with EtOAc (3×10 mL). The combined organic layers were concentrated to dryness, then purified by silica gel chromatography (100% heptane to 100% EtOAc) to give a white solid, N-(2-chloro-5-(1-methyl-1H-pyrazol-3-yl)pyridin-4-yl)-2-(1,1-difluoroethyl)-6-methoxypyrimidin-4-amine (30 mg, 19.9% yield). LCMS m/z=381.3 [M+H]⁺. 1H NMR (DMSO-d₆) δ: 11.60 (s, 1H), 8.84 (s, 1H), 8.77 (s, 1H), 8.01-7.89 (m, 1H), 7.09-6.94 (m, 1H), 6.67 (s, 1H), 4.05 (s, 3H), 3.98 (s, 3H), 2.08 (s, 6H), 1.07 (s, 2H).

### Preparation 120

### N-(4-((6-(3-((tert-butyldimethylsilyl)oxy)-3-methylcyclobutoxy)-2-(1,1-difluoroethyl)pyrimidin-4-yl)amino)-5-ethoxypyridin-2-yl)acetamide

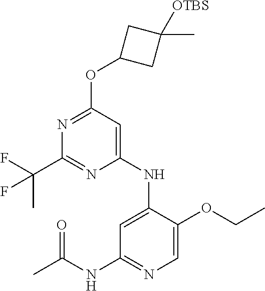 [SMILES: CCOc1cnc(NC(C)=O)cc1Nc1cc(OC2CC(C)(O[Si](C)(C)C(C)(C)C)C2)nc(C(C)(F)F)n1]

To a solution of 4-(3-((tert-butyldimethylsilyl)oxy)-3-methylcyclobutoxy)-6-chloro-2-(1,1-difluoroethyl)pyrimidine (Preparation 116, 100 mg, 0.512 mmol) in dioxane (5 mL) was added N-(4-amino-5-ethoxypyridin-2-yl)acetamide hydrochloride (Preparation 62, 221 mg, 0.563 mmol), Brettphos Pd G3 (46.4 mg, 0.0512 mmol) and Cs₂CO₃ (334 mg, 1.02 mmol). The mixture was stirred at 100° C. for 2 h under N₂ then diluted with water (15 mL) and extracted with EtOAc (20 mL×3). The combined organic phase was washed with brine (60 mL), dried over anhydrous Na₂SO₄, filtered and concentrated. The crude was purified by chromatography on silica gel (DCM/MeOH 1/0 to 10/1) to give N-(4-((6-(3-((tert-butyldimethylsilyl)oxy)-3-methylcyclobutoxy)-2-(1,1-difluoroethyl)pyrimidin-4-yl)amino)-5-ethoxypyridin-2-yl)acetamide (225 mg, crude) as a yellow solid. LCMS m/z=552.3 [M+H]⁺. ¹H NMR: (400 MHz, CDCl₃) δ ppm: 8.73 (s, 1H), 7.95 (s, 1H), 7.80 (s, 1H), 7.51 (s, 1H), 6.43 (s, 1H), 4.89-4.82 (m, 1H), 4.17 (q, J=6.8 Hz, 2H), 2.66-2.61 (m, 2H), 2.35-2.29 (m, 2H), 2.19 (s, 3H), 2.06 (t, J=18.4 Hz, 3H), 1.49 (t, J=7.2 Hz, 1H), 1.41 (s, 3H), 0.88 (s, 9H), 0.08 (s, 6H).

### Preparation 121

### N-(4-((2-(1,1-difluoroethyl)-6-(prop-1-en-2-yl)pyrimidin-4-yl)amino)-5-methoxypyridin-2-yl)acetamide

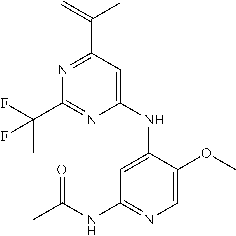 [SMILES: C=C(C)c1cc(Nc2cc(NC(C)=O)ncc2OC)nc(C(C)(F)F)n1]

To a mixture of 4-chloro-2-(1,1-difluoroethyl)-6-(prop-1-en-2-yl)pyrimidine (Preparation 95, 180 mg, 0.823 mmol), N-(4-amino-5-methoxypyridin-2-yl)acetamide hydrochloride (Preparation 61, 179 mg, 0.988 mmol) and Cs₂CO₃ (536 mg, 1.65 mmol) in DMF (4 mL) was added BrettPhos Pd G3 (74.6 mg, 0.823 mmol) at 20° C. The reaction mixture was stirred at 70° C. for 2 h. The mixture was concentrated and the residue was purified by prep-HPLC (Method C, Gradient: 42 to 72%) to give N-(4-((2-(1,1-difluoroethyl)-6-(prop-1-en-2-yl)pyrimidin-4-yl)amino)-5-methoxypyridin-2-yl)acetamide (60 mg, 20.1% yield) as a white solid. LCMS m/z=364.2 [M+H]⁺.

### Preparation 122

### N-(4-((2-(1,1-difluoroethyl)-6-(prop-1-en-2-yl)pyrimidin-4-yl)amino)-5-ethoxypyridin-2-yl)acetamide

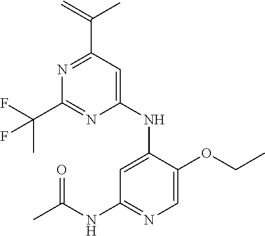 [SMILES: C=C(C)c1cc(Nc2cc(NC(C)=O)ncc2OCC)nc(C(C)(F)F)n1]

N-(4-((2-(1,1-Difluoroethyl)-6-(prop-1-en-2-yl)pyrimidin-4-yl)amino)-5-ethoxypyridin-2-yl)acetamide was obtained as a white solid, 60 mg, 17.4% yield, from 4-chloro-2-(1,1-difluoroethyl)-6-(prop-1-en-2-yl)pyrimidine (Preparation 95) and N-(4-amino-5-ethoxypyridin-2-yl)acetamide hydrochloride (Preparation 62), following a similar procedure to that described in Preparation 121, except HPLC (Method B, Gradient 37 to 66%) was used. LCMS m/z=378.3 [M+H]⁺

### Preparation 123

### 6-methoxy-5-(1-methyl-1H-pyrazol-3-yl)pyridin-2-amine

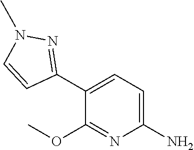

To a mixture of 5-bromo-6-methoxy-pyridin-2-amine (55 mg, 0.271 mmol), Pd(dppf)Cl₂ (15.28 mg, 0.0209 mmol), K₂CO₃ (122.22 mg, 0.884 mmol) and 1-methyl-3-(4,4,5,5-tetramethyl-1,3,2-dioxaborolan-2-yl)pyrazole (76.39 mg, 0.367 mmol) was added dioxane (2 mL) and water (1 mL) and the reaction stirred at rt for 10 mins while degassing by bubbling N₂. The vial was sealed and heated to 90° C. for 1 h. The reaction was diluted with water (10 mL), extracted with EtOAc (4×10 mL) and the combined organic layers concentrated in vacuo. The crude product was purified by silica gel chromatography (heptane to EtOAc) to give a clear colorless thick oil, 6-methoxy-5-(1-methyl-1H-pyrazol-3-yl)pyridin-2-amine (45 mg, 81.3% yield). LCMS m/z=205.2 [M+H]⁺.

### Preparation 124

### 6-chloro-2-methoxy-3-(1-methyl-1H-pyrazol-3-yl)pyridine

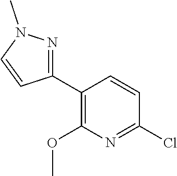

A mixture of 3-bromo-6-chloro-2-methoxy-pyridine (100 mg, 0.45 mmol), Pd(dppf)Cl₂ (25 mg, 34.2 umol), K₂CO₃ (200 mg, 1.45 mmol) and 1-methyl-3-(4,4,5,5-tetramethyl-1,3,2-dioxaborolan-2-yl)pyrazole (125 mg, 0.601 mmol) in dioxane (2 mL) and water (1 mL) was stirred at rt for 10 mins while degassing by bubbling N₂. The vial was sealed and heated to 90° C. for 1 h. The reaction was diluted with water (10 mL), extracted with EtOAc (4×10 mL) and the combined organic layers concentrated in vacuo. The residue was purified by silica gel chromatography (heptane to EtOAc) to give 6-chloro-2-methoxy-3-(1-methylpyrazol-3-yl)pyridine (73 mg, 72.6% yield) as a thick oil. LCMS m/z=404.2 [M+H]⁺. ¹H NMR (DMSO-d₆) δ: 8.25 (d, J=7.6 Hz, 1H), 7.73-7.82 (m, 1H), 7.11-7.19 (m, 1H), 6.70-6.78 (m, 1H), 3.97 (s, 3H), 3.90 (s, 3H).

### Preparation 125

### 4-chloro-2-(1,1-difluoroethyl)-6-vinylpyrimidine

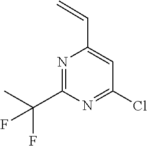

A suspension of potassium vinyltrifluoroborate (1.38 g, 10.33 mmol) and 4,6-dichloro-2-(1,1-difluoroethyl)pyrimidine (Preparation 91, 2 g, 9.39 mmol) in MeCN (40 mL) and water (10 mL) was sparged with N₂ for 10 min. K₂CO₃ (2.60 g, 18.8 mmol) and Pd(PPh₃)₄ (185 mg, 0.160 mmol) were added and the resulting mixture was heated to reflux for 18 h. The reaction was concentrated under reduced pressure and the residue was partitioned between DCM and water. The layers were separated and the aqueous was extracted with DCM (2×). The combined organic extracts were dried (MgSO₄), filtered and concentrated in vacuo. The residue was purified by silica gel chromatography (0-30% EtOAc in heptanes) to give 4-chloro-2-(1,1-difluoroethyl)-6-vinylpyrimidine (778 mg, 40% yield) as a colorless liquid. LCMS m/z=205.0 [M+H]⁺.

### Preparation 126

### 6-chloro-N-methoxy-N,4-dimethylpicolinamide

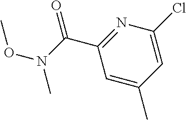

To a solution of 6-chloro-4-methylpicolinic acid (1.0 g, 5.83 mmol) in DCM (10 mL) was added DMF (42.6 mg, 0.583 mmol), followed by dropwise addition of oxalyl chloride (791.5 mg, 6.24 mmol). After 1.2 h, N,O-dimethylhydroxylamine (886.8 mg, 9.09 mmol) was added and the reaction mixture was cooled to 0° C. Pyridine (2.1 g, 26.81 mmol) was added and the reaction mixture was allowed to warm to rt and stirred for 72 h. The reaction mixture was quenched by the addition of water and the layers were separated. The aqueous phase was extracted with DCM (10 mL×3) and the combined organic layer was washed with brine (20 mL), dried with Na₂SO₄, filtered and concentrated. The crude was purified by column chromatography (PE/EtOAc=3/1 to 0/1) on silica gel to give 6-chloro-N-methoxy-N,4-dimethylpicolinamide (600 mg, 48.0% yield) as yellow oil. LCMS m/z=215.1 [M+H]⁺

### Preparation 127

### 1-(6-chloro-4-methylpyridin-2-yl)ethan-1-one

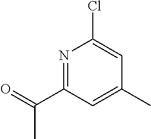

Under N₂, to a solution of 6-chloro-N-methoxy-N,4-dimethylpicolinamide (Preparation 126, 600 mg, 2.80 mmol) in THF (10.0 mL) was added MeMgBr (3 M, 3.7 mL, 11.1 mmol) slowly at −60° C. and the mixture was stirred at 0° C. for 5 h under N₂. The reaction mixture was quenched by the addition of saturated aqueous NH₄Cl solution and extracted with EtOAc (80.0 mL×3). The combined organic layers were washed with brine (100 mL), dried over Na₂SO₄, filtered and concentrated to give a residue which was purified on silica gel column chromatography (PE/EtOAc=10/1 to 3/1) to give 1-(6-chloro-4-methylpyridin-2-yl)ethan-1-one (460 mg, 97.0% yield) as a white solid. LCMS m/z=170.1 [M+H]⁺

### Preparation 128

### 2-chloro-6-(1,1-difluoroethyl)-4-methylpyridine

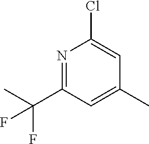

To a solution of 1-(6-chloro-4-methylpyridin-2-yl)ethan-1-one (Preparation 127, 460.0 mg, 2.71 mmol) in DCM (10 mL) was added DAST (5.0 mL, 37.84 mmol) and the solution stirred at 60° C. for 12 h. The reaction was concentrated and diluted with H₂O (10 mL). The mixture was extracted with DCM (10 mL×2) and washed with brine (10 mL×2). The combined organic phase was concentrated and purified by column chromatography (PE/EtOAc=15/1 to 5/1) on silica gel to give 2-chloro-6-(1,1-difluoroethyl)-4-methylpyridine (379.0 mg, 72.9% yield) as yellow oil. LCMS m/z=191.9 [M+H]⁺

### Preparation 129

### 2-(6-bromopyridin-2-yl)propan-2-ol

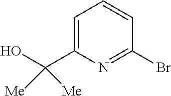 [SMILES: CC(C)(O)c1cccc(Br)n1]

To a solution of 1-(6-bromopyridin-2-yl)ethan-1-one (1 g, 5.0 mmol) in THF (10 mL) was added MeMgBr (3 M, 2.50 mL, 7.5 mmol) slowly at 0° C. and the reaction stirred for 12 h at rt under N₂. The reaction was concentrated, diluted with H₂O (50 mL) and extracted with EtOAc (20 mL×3). The combined organic phase was washed with brine (20 mL×2), dried over Na₂SO₄ and filtered. The mixture was concentrated and the residue was purified by chromatography on silica gel (PE/EtOAc=3/1) to give 2-(6-bromopyridin-2-yl)propan-2-ol (910 mg, 84% yield) as a white oil. ¹H NMR: (400 MHz, CDCl₃) δ ppm: 7.57-7.53 (m, 1H), 7.39-7.26 (m, 2H), 4.06 (br s, 1H), 1.54 (s, 6H).

### Preparation 130

### 2-bromo-6-(2-fluoropropan-2-yl)pyridine

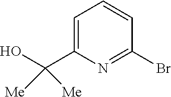 [SMILES: CC(C)(O)c1cccc(Br)n1]

To a solution of 2-(6-bromopyridin-2-yl)propan-2-ol (Preparation 129, 200 mg, 0.926 mmol) in DCM (3.0 mL) was added DAST (298.4 mg, 1.85 mmol) and the reaction was stirred at 20° C. for 12 h. The mixture was quenched with aq. Na₂CO₃ (20 mL) and extracted with DCM (20 mL×2). The combined organic phase was washed with brine (20 mL×2), dried over Na₂SO₄ and filtered. The filtrate was concentrated under reduced pressure and the residue was purified by chromatography on silica gel (PE/EtOAc=3/1) to give 2-bromo-6-(2-fluoropropan-2-yl)pyridine (190 mg, 94.1% yield) as a yellow oil. 1H NMR: (400 MHz, CDCl₃) δ ppm: 7.58-7.49 (m, 2H), 7.38 (d, J=7.6 Hz, 1H), 1.73 (s, 3H), 1.67 (s, 3H).

### Preparation 131

### 2-bromo-6-(1,1-difluoroethyl)-4-methoxypyridine

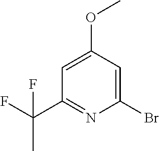

2-Bromo-6-(1,1-difluoroethyl)-4-methoxypyridine was obtained as a yellow oil, 420 mg, crude, from 1-(6-bromo-4-methoxypyridin-2-yl)ethan-1-one, following a similar procedure to that described in Preparation 130. LCMS m/z=252.0 [M+H]⁺

### Preparation 132

### 1-(2-chloro-6-methylpyrimidin-4-yl)ethan-1-one

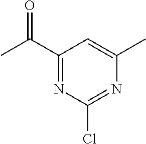

To a solution of methyl 2-chloro-6-methylpyrimidine-4-carboxylate (985 mg, 5.28 mmol) in THF (10 mL) was added MeMgBr (3 M, 1.76 mL) at 0° C. under N₂. The reaction mixture was stirred for 5 h, then hydrolysed with H₂O (30 mL) and extracted with EtOAc (3×20 mL). The combined organic phase was concentrated under reduced pressure. The residue was purified by chromatography on silica gel (PE/EtOAc 5/1) to give 1-(2-chloro-6-methylpyrimidin-4-yl)ethan-1-one (147 mg, 16.3% yield) as white solid. ¹H NMR: (400 MHz, CDCl₃) δ ppm: 7.69 (s, 1H), 2.70 (s, 3H), 2.63 (s, 3H).

### Preparation 133

### 2-chloro-4-(1,1-difluoroethyl)-6-methylpyrimidine

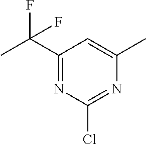

2-Chloro-4-(1,1-difluoroethyl)-6-methylpyrimidine was obtained as a yellow oil, 114 mg, 84.1% yield, from 1-(2-chloro-6-methylpyrimidin-4-yl)ethan-1-one (Preparation 132) and DAST following the procedure described in Preparation 128. ¹H NMR: (500 MHz, CDCl₃) δ ppm: 7.42 (s, 1H), 2.62 (s, 3H), 1.98 (t, J=19.0 Hz, 3H).

### Preparation 134

### 4-(benzyloxy)-2-chloro-6-methylpyrimidine

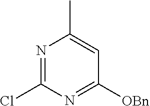 [SMILES: Cc1cc(OCc2ccccc2)nc(Cl)n1]

Under N₂, to a solution of 2,4-dichloro-6-methylpyrimidine (10 g, 61.35 mmol) in THF (60 mL) and DMF (20 mL) was added t-BuOK (1 M, 61.35 mL) and benzyl alcohol (13.27 g, 122.7 mmol) dropwise at −70° C. and the mixture was stirred at −70° C. for 2 h. The reaction mixture was quenched by the addition of saturated aqueous NH₄Cl solution (10 mL). The mixture was concentrated under reduced pressure and extracted with EtOAc (60 mL×3). The combined organic layers were washed with brine (50 mL), dried over Na₂SO₄, filtered and concentrated to give a residue, which was purified on silica gel column chromatography (PE/EtOAc=9/1) to give 4-(benzyloxy)-2-chloro-6-methylpyrimidine (4.9 g, 34.0% yield) as a colorless oil. 1H NMR (500 MHz, MeOH-d₄) ppm: 7.34-7.45 (m, 5H), 6.72 (s, 1H), 5.41 (s, 2H), 2.40 (s, 3H).

### Preparation 135

### Methyl 4-(benzyloxy)-6-methylpyrimidine-2-carboxylate

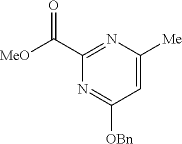 [SMILES: COC(=O)c1nc(C)cc(OCc2ccccc2)n1]

To a solution of 4-(benzyloxy)-2-chloro-6-methylpyrimidine (Preparation 134, 4.5 g, 19.17 mmol) and TEA (9.7 g, 95.87 mmol) in MeOH (50 mL) was added Pd(dppf)Cl₂ (1.40 g, 1.92 mmol) and the reaction was stirred at 80° C. for 16 h under CO (50 psi). The mixture was concentrated under reduced pressure to give a residue which was purified on silica gel column chromatography (PE/EtOAc=9/1 to 5/1) to give methyl 4-(benzyloxy)-6-methylpyrimidine-2-carboxylate (2.8 g, 56%) as a green oil. ¹H NMR (500 MHz, MeOH-d₄) δ: 7.28-7.53 (m, 5H), 6.94 (s, 1H), 5.50 (s, 2H), 4.00 (s, 3H), 2.50 (s, 3H).

### Preparation 136

### 1-(4-(benzyloxy)-6-methylpyrimidin-2-yl)ethan-1-one

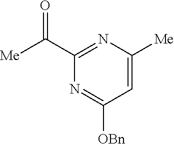 [SMILES: CC(=O)c1nc(C)cc(OCc2ccccc2)n1]

1-(4-(Benzyloxy)-6-methylpyrimidin-2-yl)ethan-1-one was obtained as a yellow oil, 312 mg, 13%, from methyl 4-(benzyloxy)-6-methylpyrimidine-2-carboxylate (Preparation 135), and CH₃MgBr, following a similar procedure to that described in Preparation 129. LCMS m/z=243.0 [M+H]⁺.

### Preparation 137

### 4-(benzyloxy)-2-(1,1-difluoroethyl)-6-methylpyrimidine

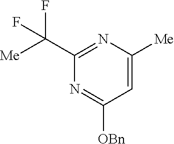 [SMILES: Cc1cc(OCc2ccccc2)nc(C(C)(F)F)n1]

4-(Benzyloxy)-2-(1,1-difluoroethyl)-6-methylpyrimidine was obtained as a yellow oil, 208 mg, 61% from 1-(4-(benzyloxy)-6-methylpyrimidin-2-yl)ethan-1-one (Preparation 136), following the procedure described in Preparation 128. LCMS m/z=265.1 [M+H]⁺.

### Preparation 138

### 2-(1,1-difluoroethyl)-6-methylpyrimidin-4-ol

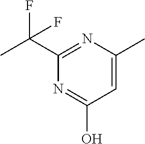

A mixture of 4-(benzyloxy)-2-(1,1-difluoroethyl)-6-methylpyrimidine (Preparation 137, 208 mg, 0.8 mmol) and TFA (1 mL) was stirred at 100° C. for 12 h. The mixture was concentrated under reduced pressure to give a residue, which was purified on silica gel column chromatography (DCM/MeOH=19/1) to give 2-(1,1-difluoroethyl)-6-methylpyrimidin-4-ol (124 mg, 90%) as a yellow solid. ¹H NMR (500 MHz, MeOH-d₄) δ: 6.37 (s, 1H), 2.34 (s, 3H), 1.98 (t, 3H).

### Preparation 139

### 4-chloro-2-(1,1-difluoroethyl)-6-methylpyrimidine

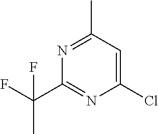

A mixture of 2-(1,1-difluoroethyl)-6-methylpyrimidin-4-ol (Preparation 138, 124 mg, 0.7 mmol) and POCl₃(1 mL) was stirred at 100° C. for 1 h. The reaction mixture was added to ice water (5 mL) dropwise and extracted with EtOAc (10 mL×3). The combined organics were washed with brine (15 mL), dried (Na₂SO₄) and evaporated to dryness in vacuo to give 4-chloro-2-(1,1-difluoroethyl)-6-methylpyrimidine (86 mg, 63%) as a brown oil. LCMS m/z=193.1 [M+H]+.

### Preparation 140

### 4-(benzyloxy)-2-(1,1-difluoroethyl)-6-(prop-1-en-2-yl)pyrimidine

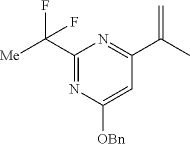 [SMILES: C=C(C)c1cc(OCc2ccccc2)nc(C(C)(F)F)n1]

Step 1: To a solution of 4,6-dichloro-2-(1,1-difluoroethyl)pyrimidine (Preparation 91, 5.0 g, 23.47 mmol) in THF (40 mL) was added NaH (1.9 g, 46.95 mmol, 60% purity) and BnOH (2.7 g, 24.55 mmol) and the reaction stirred at 25° C. for 1 h. The reaction mixture was concentrated in vacuo to give crude 4-(benzyloxy)-6-chloro-2-(1,1-difluoroethyl)pyrimidine. Step 2: To 4-(benzyloxy)-6-chloro-2-(1,1-difluoroethyl)pyrimidine (6.7 g, 23.5 mmol) was added THF and water (10 mL), then 4,4,5,5-tetramethyl-2-(prop-1-en-2-yl)-1,3,2-dioxaborolane (7.9 g, 46.9 mmol), K₂CO₃ (10.0 g, 72.4 mmol) and Pd(dppf)Cl₂ (1.7 g, 2.35 mmol) and the reaction stirred at 70° C. for 2 h under N₂. The mixture was quenched with H₂O (100 mL) and extracted with EtOAc (50 mL×3). The combined organic layers were washed with brine (100 mL), dried over Na₂SO₄, filtered and concentrated under vacuum to give the crude, which was purified on silica gel column chromatography (PE/EtOAc=1/0 to 10/1) to give 4-(benzyloxy)-2-(1,1-difluoroethyl)-6-(prop-1-en-2-yl)pyrimidine (6.0 g, yield: 88%, two steps) as a white solid. ¹H NMR: (400 MHz, DMSO-d₆) δ ppm: 7.52-7.49 (m, 2H), 7.40-7.35 (m, 3H), 7.17 (s, 1H), 6.26 (s, 1H), 5.52-5.51 (m, 1H), 5.47 (s, 2H), 5.47-5.41 (s, 1H), 2.11-1.99 (m, 3H), 1.73-1.72 (m, 3H).

### Preparation 141

### 2-(1,1-difluoroethyl)-6-isopropylpyrimidin-4-ol

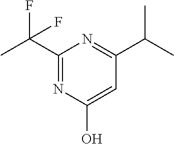

To a solution of 4-(benzyloxy)-2-(1,1-difluoroethyl)-6-(prop-1-en-2-yl)pyrimidine (Preparation 140, 6.0 g, 20.67 mmol) in EtOH (60 mL) was added Pd/C (4.4 g, 4.13 mmol, 10% purity) and the reaction mixture was stirred at 65° C. under H₂ at 15 Psi for 4 h. The mixture was quenched with H₂O (100 mL) and extracted with EtOAc (50 mL×3). The combined organic layers were washed with brine (100 mL), dried over Na₂SO₄, filtered and concentrated under vacuum. The crude product was purified on silica gel column chromatography (PE/EtOAc=1/0 to 1/1) to give 2-(1,1-difluoroethyl)-6-isopropylpyrimidin-4-ol (3.0 g, 72.5% yield) as a white solid. ¹H NMR: (400 MHz, DMSO-d₆) δ ppm 12.71 (br s, 1H), 6.46 (br s, 1H), 2.87-2.79 (m, 1H), 2.03-1.91 (m, 3H), 1.17 (d, J=6.8 Hz, 6H).

### Preparation 142

### 4-chloro-2-(1,1-difluoroethyl)-6-isopropylpyrimidine

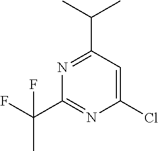

To a solution of 2-(1,1-difluoroethyl)-6-isopropylpyrimidin-4-ol (Preparation 141, 3.0 g, 15.0 mmol) was added POCl₃ (8.3 g, 54.2 mmol) and the reaction was stirred at 100° C. for 1 h. The mixture was concentrated, diluted with DCM (100 mL) and aq.NaHCO₃ (200 mL) slowly added. The mixture was stirred at 25° C. for 1 h, the layers separated and the aqueous extracted with DCM (20 mL×3). The combined organic layers were washed with brine (50 mL), dried over Na₂SO₄, filtered and concentrated to give 4-chloro-2-(1,1-difluoroethyl)-6-isopropylpyrimidine (3.1 g, 93.7% yield) as a yellow oil. ¹H NMR (400 MHz, CDCl₃) δ ppm: 7.20 (d, J=2.4 Hz, 1H), 3.07-2.98 (m, 1H), 2.03-1.94 (m, 3H), 1.27-1.25 (m, 6H).

### Preparation 143

### 4-(benzyloxy)-2-chloropyrimidine

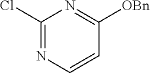 [SMILES: Clc1nccc(OCc2ccccc2)n1]

4-(Benzyloxy)-2-chloropyrimidine was obtained as a white solid, 4.8 g, 64.8%, from 2,4-dichloropyrimidine, following a similar procedure to that described in Preparation 134. 1H NMR (400 MHz, CDCl₃) δ ppm: 8.31 (d, J=5.6 Hz, 1H), 7.37-7.46 (m, 5H), 6.70 (d, J=5.6 Hz, 1H), 5.43 (s, 2H).

### Preparation 144

### methyl 4-(benzyloxy)pyrimidine-2-carboxylate

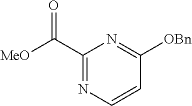 [SMILES: COC(=O)c1nccc(OCc2ccccc2)n1]

To a solution of 4-(benzyloxy)-2-chloropyrimidine (Preparation 143, 9.5 g, 21.53 mmol) in MeOH (100 mL) was added Pd(dppf)Cl₂ (315.0 mg, 0.43 mmol) and TEA (4.4 g, 43.1 mmol). The resulting mixture was stirred at 80° C. for 16 h under CO (50 psi). The mixture was concentrated and was purified by chromatography (PE/EtOAc=3/1) on silica gel to give methyl 4-(benzyloxy)pyrimidine-2-carboxylate (3.6 g, 68.5% yield) as a yellow solid. ¹H NMR: (400 MHz, CDCl₃) δ ppm: 8.59 (d, J=5.6 Hz, 1H), 7.42-7.50 (m, 2H), 7.35-7.41 (m, 3H), 6.91 (d, J=6.0 Hz, 1H), 5.53 (s, 2H), 4.05 (s, 3H).

### Preparation 145

### 1-(4-(benzyloxy)pyrimidin-2-yl)ethan-1-one

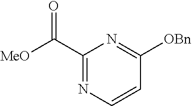 [SMILES: COC(=O)c1nccc(OCc2ccccc2)n1]

1-(4-(Benzyloxy)pyrimidin-2-yl)ethan-1-one was obtained as a colorless oil, 288 mg, 21%, from methyl 4-(benzyloxy)pyrimidine-2-carboxylate (Preparation 144), following a similar procedure to that described in Preparation 129. ¹H NMR (400 MHz, CDCl₃) δ: 8.61 (d, 1H), 7.49-7.35 (m, 5H), 6.90 (d, 1H), 5.52 (s, 2H), 2.75 (s, 3H).

### Preparation 146

### 4-(benzyloxy)-2-(1,1-difluoroethyl)pyrimidine

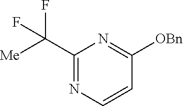 [SMILES: CC(F)(F)c1nccc(OCc2ccccc2)n1]

4-(Benzyloxy)-2-(1,1-difluoroethyl)pyrimidine was obtained as a yellow oil, 537 mg, 76%, from 1-(4-(benzyloxy)pyrimidin-2-yl)ethan-1-one (Preparation 145) following a similar procedure to that described in Preparation 130. ¹H NMR (500 MHz, CDCl₃) δ: 8.52 (d, 1H), 7.48-7.35 (m, 5H), 6.81 (d, 1H), 5.48 (s, 2H), 2.03 (t, 3H).

### Preparation 147

### 2-(1,1-difluoroethyl)pyrimidin-4-ol

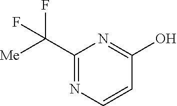 [SMILES: CC(F)(F)c1nccc(O)n1]

A solution of 4-(benzyloxy)-2-(1,1-difluoroethyl)pyrimidine (Preparation 146, 537.1 mg, 2.15 mmol) in TFA (4.47 g, 39.2 mmol) was stirred at 100° C. for 12 h. The mixture was purified by prep-HPLC-D (0-20% MeCN) to give 2-(1,1-difluoroethyl)pyrimidin-4-ol as a white solid (230 mg, 67%). ¹H NMR (400 MHz, CDCl₃) δ: 7.99 (d, 1H), 6.55 (d, 1H), 2.03 (t, 3H).

### Preparation 148

### 4-chloro-2-(1,1-difluoroethyl)pyrimidine

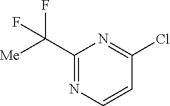 [SMILES: CC(F)(F)c1nccc(Cl)n1]

A solution of 2-(1,1-difluoroethyl)pyrimidin-4-ol (Preparation 147, 100 mg, 0.625 mmol) in POCl₃ (2 mL) was stirred at 100° C. for 2 h. The reaction mixture was concentrated, the residue diluted with H₂O (10 mL) and extracted with EtOAc (3×10 mL). The combined organics were dried (Na₂SO₄) and concentrated to give 4-chloro-2-(1,1-difluoroethyl)pyrimidine as a yellow oil (85 mg, 76%). ¹H NMR (400 MHz, CDCl₃) δ: 8.74 (d, 1H), 7.44 (d, 1H), 2.07 (t, 3H).

### Preparation 149

### 2-(4-chloropyrimidin-2-yl)propan-2-ol

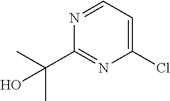

To a solution of 4-chloro-2-iodopyrimidine (4.2 g, 17.64 mmol) in toluene (10 mL) was added n-BuLi (2.5 M, 10.6 mL) slowly at −70° C. over 30 mins under N₂. Acetone (1.2 g, 21.16 mmol) was added slowly at −70° C. and the reaction then stirred for 3 h under N₂. The mixture was poured into aq.NH₄Cl (50 mL) and extracted with EtOAc (50 mL×3). The organic phase was washed with brine (30 mL), dried with Na₂SO₄, filtered and the filtrate was concentrated. The residue was purified by chromatography (PE/EtOAc=20/1 to 3/1) on silica gel to give 2-(4-chloropyrimidin-2-yl)propan-2-ol (1.5 g, 49.3% yield) as yellow oil.

LCMS m/z=173.1 [M+H]⁺

### Preparation 150

### 2-(4,6-dichloropyrimidin-2-yl)propan-2-ol

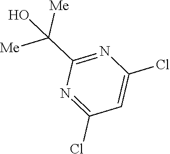 [SMILES: CC(C)(O)c1nc(Cl)cc(Cl)n1]

2-(4,6-Dichloropyrimidin-2-yl)propan-2-ol was obtained as a yellow oil, 500 mg, 66% from 4,6-dichloro-2-iodopyrimidine, following the procedure described in Preparation 149. ¹H NMR (500 MHz, CDCl₃) δ: 7.30 (s, 1H), 3.92 (br s, 1H), 1.59 (s, 6H).

### Preparation 151

### 4,6-dichloro-2-(2-fluoropropan-2-yl)pyrimidine

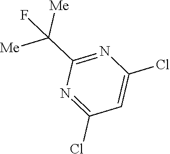 [SMILES: CC(C)(F)c1nc(Cl)cc(Cl)n1]

4,6-Dichloro-2-(2-fluoropropan-2-yl)pyrimidine was obtained as a yellow oil, 350 mg, 69% from 2-(4,6-dichloropyrimidin-2-yl)propan-2-ol (Preparation 150) and DAST following a similar procedure to that described in Preparation 130. 1H NMR (500 MHz, CDCl₃) δ: ppm 7.34 (s, 1H), 1.80 (s, 3H), 1.75 (s, 3H).

### Preparation 152

### 4-chloro-2-(1-ethoxyvinyl)-5-methoxypyrimidine

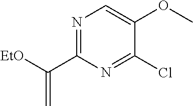

To a stirring solution of 2,4-dichloro-5-methoxypyrimidine (28.2 g, 157.7 mmol) and tributyl(1-ethoxyvinyl)stannane (66.5 g, 184.1 mmol) in DMF (980 mL, degassed with N₂ for 10 min) were added Pd(PPh₃)₂Cl₂ (3.3 g, 4.7 mmol) and TEA (19.3 g, 190.8 mmol). The resulting mixture was heated at 100° C. for 16 h. The reaction mixture was cooled to 25° C., diluted with EtOAc and filtered through silica. The filtrate was washed with saturated solution of KF (300 mL), H₂O (300 mL) and brine (300 mL) and dried over Na₂SO₄ and filtered. The filtrate was concentrated in vacuo to give 4-chloro-2-(1-ethoxyvinyl)-5-methoxypyrimidine (28 g, 83.7% yield). LCMS m/z=214.9 [M+H]+

### Preparation 153

### 1-(4-chloro-5-methoxypyrimidin-2-yl)ethan-1-one

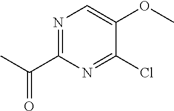

To a solution of 4-chloro-2-(1-ethoxyvinyl)-5-methoxypyrimidine (Preparation 152, 28 g, 130.5 mmol) in dioxane (450 mL) cooled to 0° C. was added HCl (2.0 M, 65.22 mL) and the solution stirred for 2 h. The reaction was diluted with water (1 L) and extracted with EtOAc (200 mL×3), the combined organic phases washed with brine (400 mL), dried with anhydrous Na₂SO₄ and concentrated. The residue was purified with silica chromatography (EtOAc: PE from 0 to 15%) to give 1-(4-chloro-5-methoxypyrimidin-2-yl)ethan-1-one (23 g, 94.5% yield) as a yellow solid. LCMS m/z=186.9 [M+H]+

### Preparation 154

### 4-chloro-2-(1,1-difluoroethyl)-5-methoxypyrimidine

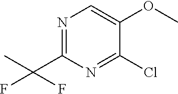

To a solution of 1-(4-chloro-5-methoxypyrimidin-2-yl)ethan-1-one (Preparation 153, 23.0 g, 123.3 mmol) in DCM (200 mL) cooled to 0° C. was added DAST (49.7 g, 308.2 mmol) and the reaction stirred for 16 h at 45° C. Saturated aq. NaHCO₃ (200 mL) was added dropwise to quench the reaction and it was extracted with DCM (200 mL), washed with brine (200 mL), dried over Na₂SO₄ and concentrate. The crude product was purified by silica gel chromatography (0˜7%=EtOAc: PE) to give 4-chloro-2-(1,1-difluoroethyl)-5-methoxypyrimidine (19 g, 73.9% yield) as a yellow solid. LCMS m/z=209.0 [M+H]+

### Preparation 155

### 4-chloro-6-cyclopropyl-2-(1,1-difluoroethyl)pyrimidine

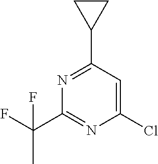

4-Chloro-6-cyclopropyl-2-(1,1-difluoroethyl)pyrimidine was obtained as a colorless oil, 310 mg, crude, from 4,6-dichloro-2-(1,1-difluoroethyl)pyrimidine (Preparation 91) and cyclopropylboronic acid following the procedure described in Preparation 94. LCMS m/z=219.1 [M+H]⁺.

### Preparation 156

### 4-chloro-6-ethyl-2-(2-fluoropropan-2-yl)pyrimidine

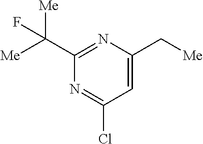 [SMILES: CCc1cc(Cl)nc(C(C)(C)F)n1]

4-Chloro-6-ethyl-2-(2-fluoropropan-2-yl)pyrimidine was obtained as a colorless oil, 300 mg, crude, from 4,6-dichloro-2-(2-fluoropropan-2-yl)pyrimidine (Preparation 151) and ethyl boronic acid, following the procedure described in Preparation 94. LCMS m/z=203.0 [M+H]⁺.

### Preparation 157

### 4-chloro-2-(2-fluoropropan-2-yl)-6-methylpyrimidine

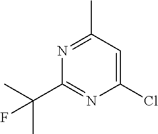

4-Chloro-2-(2-fluoropropan-2-yl)-6-methylpyrimidine was obtained as a yellow oil, 720 mg, 79.8% from 4,6-dichloro-2-(2-fluoropropan-2-yl)pyrimidine (Preparation 151) and methyl boronic acid, following a similar procedure to that described in Preparation 94. ¹H NMR: (500 MHz, CDCl₃) δ ppm: 7.13 (s, 1H), 2.56 (s, 3H), 1.79 (s, 3H), 1.75 (s, 3H).

### Preparation 158

### 4-chloro-2-(1,1-difluoroethyl)-6-(2-methoxyethoxy)pyrimidine

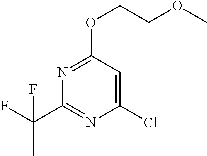 [SMILES: COCCOc1cc(Cl)nc(C(C)(F)F)n1]

To a solution of 4,6-dichloro-2-(1,1-difluoroethyl)pyrimidine (Preparation 91, 200 mg, 0.939 mmol) and Cs₂CO₃ (917.8 mg, 2.82 mmol) in DMF (5 mL) was added 2-methoxyethan-1-ol (78.59 mg, 1.03 mmol) and the mixture was stirred at 25° C. for 1 h. The reaction mixture was quenched by the addition of water and extracted with EtOAc (40 mL×3). The combined organic layers were washed with H₂O and brine (40 mL), dried over Na₂SO₄, filtered and concentrated under reduced pressure. The residue was purified on silica gel column chromatography (PE/EtOAc=10/1) to give 4-chloro-2-(1,1-difluoroethyl)-6-(2-methoxyethoxy)pyrimidine (180 mg, 75.9% yield) as a yellow oil. LCMS m/z=253 [M+H]⁺

### Preparation 159

### 2-(1,1-difluoroethyl)-4-(2-methoxyethoxy)pyridine

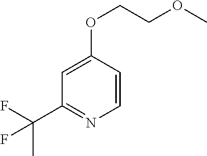

To a solution of 4-chloro-2-(1,1-difluoroethyl)pyridine (144.6 mg, 1.90 mmol) in DMF (1 mL) was added NaH (101.4 mg, 2.53 mmol, 60% purity) at 20° C. 2-Methoxyethanol (250 mg, 1.27 mmol) was added and the resulting mixture was stirred at 75° C. for 5 h. The reaction mixture was quenched by addition H₂O (20 mL) at 0° C. and extracted with EtOAc (20 mL×3). The combined organic layers were washed with brine (20 mL), dried over Na₂SO₄, filtered and concentrated under reduced pressure. The residue was purified by silica gel chromatography (PE/EtOAc=100/1 to 5/1) to give 2-(1,1-difluoroethyl)-4-(2-methoxyethoxy)pyridine (210 mg, 68.7% yield) as a colorless oil. LCMS m/z=218.2 [M+H]⁺

### Preparation 160

### (2-(1,1-difluoroethyl)-4-(2-methoxyethoxy)pyridin-1-ium-1-yl)methanide

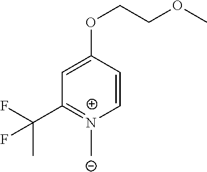

To a solution of 2-(1,1-difluoroethyl)-4-(2-methoxyethoxy)pyridine (Preparation 159, 210 mg, 0.87 mmol) in DCM (4 mL) was added m-CPBA (265 mg, 1.31 mmol) and the reaction mixture was stirred at 25° C. for 5 h under N₂. The reaction mixture was quenched with sat.NaHCO₃ (20 mL) and extracted with EtOAc (20 mL×3). The combined organic layers were washed with brine (20 mL), dried over Na₂SO₄, filtered and concentrated under reduced pressure. The residue was purified by silica gel chromatography (DCM/MeOH=100/1 to 10/1) to give (2-(1,1-difluoroethyl)-4-(2-methoxyethoxy)pyridin-1-ium-1-yl)methanide (160 mg, 71.0% yield) as a colorless oil. LCMS m/z=234.2 [M+H]⁺

### Preparation 161

### 2-chloro-6-(1,1-difluoroethyl)-4-(2-methoxyethoxy)pyridine

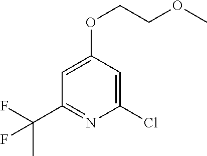 [SMILES: COCCOc1cc(Cl)nc(C(C)(F)F)c1]

A solution of (2-(1,1-difluoroethyl)-4-(2-methoxyethoxy)pyridin-1-ium-1-yl)methanide (Preparation 160, 160 mg, 0.618 mmol) in POCl₃ (2 mL) was stirred at 95° C. for 3 h under N₂. The reaction mixture was quenched with sat.NaHCO₃ (20 mL) and extracted with EtOAc (20 mL×3). The combined organic layers were washed with brine (20 mL), dried over Na₂SO₄, filtered and concentrated under reduced pressure. The residue was purified by silica gel chromatography (from PE/EtOAc=100/1 to 25/2) to give 2-chloro-6-(1,1-difluoroethyl)-4-(2-methoxyethoxy)pyridine (110 mg, 63.7% yield) as a colorless oil. LCMS m/z=252.1 [M+H]⁺

### Preparation 162

### 4-chloro-2-(1,1-difluoroethyl)-6-isopropoxypyrimidine

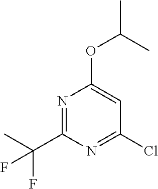

To a solution of propan-2-ol (282 mg, 4.69 mmol) in THF (5.0 mL) was added NaH (103 mg, 2.58 mmol, 60% purity) at 0° C. The reaction mixture was stirred at 25° C. for 30 min before addition of 4,6-dichloro-2-(1,1-difluoroethyl)pyrimidine (Preparation 91, 500 mg, 2.35 mmol). The reaction mixture was stirred at 25° C. for 16 h, then quenched with H₂O (10 mL) and extracted with EtOAc (3×8 mL). The organic phase was washed with brine (15 mL), dried with Na₂SO₄, filtered, concentrated, then purified by chromatography on silica gel (PE/EtOAc 15/1 to 5/1) to give 4-chloro-2-(1,1-difluoroethyl)-6-isopropoxypyrimidine (150 mg, 27.0% yield) as colorless oil. LCMS m/z=237.1 [M+H]⁺.

### Preparation 163

### 4-chloro-6-cyclopropoxy-2-(1,1-difluoroethyl)pyrimidine

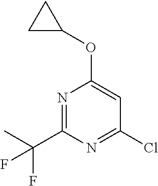

A mixture of cyclopropanol (300 mg, 5.17 mmol) in THF (10 mL) was cooled on a dry ice/IPA bath, then NaH (240 mg, 6.00 mmol, 60% purity) was added and stirred for 5 mins, then allowed to warm to rt and stirred for 30 mins. The slurry was cooled on a dry ice/IPA bath, then 4,6-dichloro-2-(1,1-difluoroethyl)pyrimidine (Preparation 91, 1.1 g, 5.16 mmol) in THF (5 mL) was added and the reaction was stirred for 2 h. The reaction was quenched by addition of NH₄Cl (10 mL), then diluted with hexane (5 mL) and the phases separated. The aqueous phase was extracted with EtOAc:heptane (15 mL 1:1) and the combined organic layers were concentrated to dryness. The crude was purified via silica gel chromatography (40 g, heptane to 15% EtOAc in heptane) to give chloro-6-(cyclopropoxy)-2-(1,1-difluoroethyl)pyrimidine as a clear colorless oil, 4-(785 mg, 49.2% yield). LCMS m/z=235.1 [M+H]⁺.

### Preparation 164

### 4-chloro-2-(1,1-difluoroethyl)-6-ethoxypyrimidine

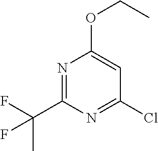

To a solution of 4,6-dichloro-2-(1,1-difluoroethyl)pyrimidine (Preparation 91, 200 mg, 0.939 mmol) in DMF (4.0 mL) was added EtONa (95.9 mg, 1.41 mmol). The reaction mixture was stirred at 25° C. for 1 h, then concentrated, treated with water (10 mL) and extracted with EtOAc (3×30 mL). The combined organic phase was washed with brine (20 mL), dried over anhydrous Na₂SO₄, filtered and concentrated under reduced pressure. The crude was purified by chromatography on silica gel (PE/EtOAc 1/0 to 10/1) to give 4-chloro-2-(1,1-difluoroethyl)-6-ethoxypyrimidine (200 mg, 95.7% yield) as a white solid. LCMS m/z=223.0 [M+H]⁺.

### Preparation 165

### 4-chloro-6-cyclopropoxy-2-(1,1-difluoroethyl)pyrimidine

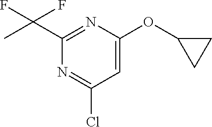

To a solution of 4,6-dichloro-2-(1,1-difluoroethyl)pyrimidine (Preparation 91, 20 g, 564 mmol) and cyclopropanol (45.8 g, 789 mmol) in THF (12 mL) was added t-BuONa (70.4 g, 732 mmol), dissolved in THF (20 mL). The reaction mixture was stirred at 25° C. for 3 h. The resulting product was taken up in THF and filtered and the filtrate concentrated in vacuo. The residue was purified by chromatography on silica gel (PE/EtOAc 1/0 to 10/1) to give 4-chloro-6-cyclopropoxy-2-(1,1-difluoroethyl)pyrimidine as colorless oil. LCMS m/z=234.9 [M+H]⁺.

### Preparation 166

### 4-chloro-6-cyclopropoxy-2-(trifluoromethyl)pyrimidine

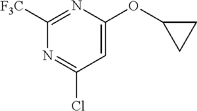 [SMILES: FC(F)(F)c1nc(Cl)cc(OC2CC2)n1]

4-Chloro-6-cyclopropoxy-2-(trifluoromethyl)pyrimidine was obtained as a colorless liquid, from 4,6-dichloro-2-(trifluoromethyl)pyrimidine and cyclopropanol, following a similar procedure to that described in Preparation 165. LCMS m/z=238.8 [M+H]⁺

### Preparation 167

### 2-(1,1-difluoropropyl)pyrimidine-4,6-diol

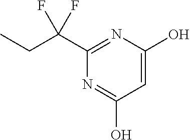

To a solution of malonamide (0.210 g, 2.06 mmol) in EtOH (5 mL) was added t-BuONa (692 mg, 7.20 mmol) and the mixture was stirred at 25° C. for 30 min. Ethyl 2,2-difluorobutanoate (642 mg, 4.22 mmol) was added and the reaction was heated to 100° C. for 1 d. The mixture was cooled to rt and 4M HCl (2.57 mL, 10.3 mmol, 4 M in dioxane) was added carefully. The precipitate formed was removed by filtration and the filtrate was evaporated to dryness. The residue was partitioned between EtOAc and water (minimum) and separated. The organics were dried over MgSO₄, filtered and evaporated. The residue was purified by silica gel chromatography (0-100% 3:1 EtOAc/EtOH in heptanes) to give 2-(1,1-difluoropropyl)pyrimidine-4,6-diol (0.104 g, 27% yield) as a white solid. LCMS m/z=191.0 [M+H]⁺.

### Preparation 168

### 4,6-dichloro-2-(1,1-difluoropropyl)pyrimidine

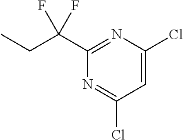

4,6-Dichloro-2-(1,1-difluoropropyl)pyrimidine was obtained as a colorless oil, 64 mg, 52%, from 2-(1,1-difluoropropyl)pyrimidine-4,6-diol (Preparation 167) and POCl₃, following a similar procedure to that described in Preparation 91. ¹H NMR (400 MHz, CDCl₃) δ ppm 7.49 (s, 1H), 2.27-2.45 (m, 2H), 1.08 (t, J=7.51 Hz, 3H).

### Preparation 169

### 4-chloro-2-(1,1-difluoropropyl)-6-methylpyrimidine

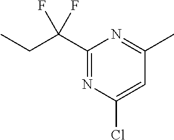

4-Chloro-2-(1,1-difluoropropyl)-6-methylpyrimidine was obtained as a colorless oil, from 4,6-dichloro-2-(1,1-difluoropropyl)pyrimidine (Preparation 168) and methylboronic acid, following a similar procedure to that described in Preparation 94. LCMS m/z=207.0 [M+H]⁺.

### Preparation 170

### 4-chloro-6-(3-methoxycyclobutoxy)-2-methylpyrimidine

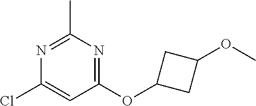

KOtBu (1.00 mL, 1.00 mmol, 1 M in THF) was added dropwise to a solution of 4,6-dichloro-2-methylpyrimidine (163 mg, 1.0 mmol) in THF (4 mL). A solution of 3-methoxycyclobutan-1-ol (102 mg, 1.0 mmol) in THF (2 mL) was added dropwise and the reaction was stirred at rt overnight. The reaction was quenched by addition of saturated NH₄Cl solution and the mixture extracted with EtOAc, washed with brine, dried over Na₂SO₄, filtered and evaporated. The residue was purified by silica gel chromatography (0-100% EtOAc in heptanes) to give 4-chloro-6-(3-methoxycyclobutoxy)-2-methylpyrimidine (14 mg, 6% yield). LCMS m/z=229.0 [M+H]⁺.

### Preparation 171

### 2-oxabicyclo[2.1.1]hexane-4-carboxamide

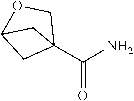

To a solution of 2-oxabicyclo[2.1.1]hexane-4-carboxylic acid (2 g, 15.61 mmol) in DCM (30 mL) under N₂ was added DMF (120 uL), followed by oxalyl chloride (2 M, 9.4 mL) and the reaction stirred at rt for 1 h. The reaction mixture was concentrated in vacuo. The residue was dissolved in DCM (20 mL), cooled to 0° C. and ammonia (7 M in MeOH, 33.45 mL) was slowly added. The mixture was stirred at rt for 2 h. The solid formed (inorganics) were filtered off, rinsed with DCM and the filtrate was concentrated. The residue was diluted with MeCN, the solid formed was collected by filtration, rinsed with MeCN and dried to give 2-oxabicyclo[2.1.1]hexane-4-carboxamide as a white solid (1.86 g, 93% yield). ¹H NMR (MeOH-d₄, 400 MHz) δ 4.50 (t, 1H, J=1.1 Hz), 3.81 (s, 2H), 2.12 (td, 2H, J=1.3, 4.9 Hz), 1.7-1.9 (m, 2H).

### Preparation 172

### 2-(2-oxabicyclo[2.1.1]hexan-4-yl)-6-methylpyrimidin-4(3H)-one

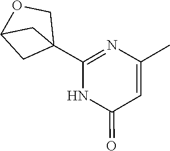

A mixture of NaOMe (3.92 mL, 19.11 mmol) (25 wt % in MeOH) in n-BuOH (4 mL) was heated to 105° C. in flask (poked with a needle open to air) while the solvent was removed. To this was slowly added a solution of methyl (E)-3-aminobut-2-enoate (550 mg, 4.78 mmol) and 2-oxabicyclo[2.1.1]hexane-4-carboxamide (Preparation 171, 1.53 g, 12.02 mmol) in MeOH (18 mL) and the reaction mixture was heated at 110° C. for 2 h. The cooled mixture was neutralized with conc. HCl, the resulting solid was filtered off and rinsed with small volume of MeOH. The filtrate was concentrated under reduced pressure and the residue was diluted with MeCN. The solid formed was collected by filtration and dried to give 4-methyl-2-(2-oxabicyclo[2.1.1]hexan-4-yl)-1H-pyrimidin-6-one (565 mg, 61% yield). LCMS m/z=193 [M+H]⁺

### Preparation 173

### 2-(2-oxabicyclo[2.1.1]hexan-4-yl)-4-chloro-6-methylpyrimidine

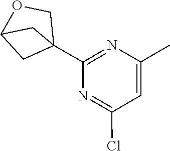

A mixture of 4-methyl-2-(2-oxabicyclo[2.1.1]hexan-4-yl)-1H-pyrimidin-6-one (Preparation 172, 560 mg, 2.91 mmol), DMF (112 μL) and POCl₃ (1.79 g, 11.65 mmol) in DCM (7 mL) was stirred at rt for 2 h. The reaction mixture was concentrated under reduced pressure, the residue diluted with EtOAc and ice and the mixture neutralized with NaHCO₃(aq). The layers were separated, the aqueous phase extracted with EtOAc and the combined organic layer was dried and concentrated to give 2-(2-oxabicyclo[2.1.1]hexan-4-yl)-4-chloro-6-methylpyrimidine (715 mg, crude) as a yellowish oil. LCMS m/z=211 [M+H]⁺

### Preparation 174

### 2-(2-oxabicyclo[2.1.1]hexan-4-yl)-6-ethylpyrimidin-4(3H)-one

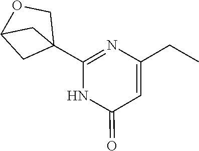

2-(2-Oxabicyclo[2.1.1]hexan-4-yl)-6-ethylpyrimidin-4(3H)-one was obtained as a white solid, from methyl (E)-3-aminopent-2-enoate (400 mg, 3.10 mmol) and 2-oxabicyclo[2.1.1]hexane-4-carboxamide (Preparation 171), following a similar procedure to that described in Preparation 172. LCMS m/z=207 [M+H]⁺

### Preparation 175

### 2-(2-oxabicyclo[2.1.1]hexan-4-yl)-4-chloro-6-ethylpyrimidine

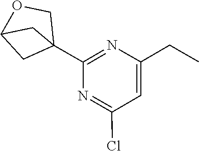

2-(2-Oxabicyclo[2.1.1]hexan-4-yl)-4-chloro-6-ethylpyrimidine was obtained as a yellow oil, 253 mg, 43%, from 2-(2-oxabicyclo[2.1.1]hexan-4-yl)-6-ethylpyrimidin-4(3H)-one (Preparation 174), following a similar procedure to that described in Preparation 173. LCMS m/z=225 [M+H]⁺

### Preparations 176 to 180

The following compounds were prepared from the appropriate carboxylic acid, following a similar 3 step synthesis to that described for the synthesis of Preparation 173.

| Preparation | |
|---|---|
| No | Name, Structure, Starting material (SM), Data |
| 176 | 2-(2-oxabicyclo[2.1.1]hexan-1-yl)-4-chloro-6- |
| | methylpyrimidine |
| | 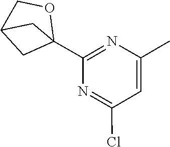 |
| | SM: 2-oxabicyclo[2.1.1 ]hexane-1-carboxylic acid |
| | LCMS m/z = 211 [M + H]⁺ |
| 177 | 4-chloro-6-methyl-2-(1-methyl-2-oxabicyclo[2.1.1] |
| | hexan-4-yl)pyrimidine |
| | 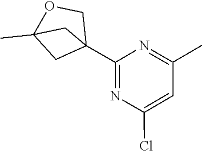 |
| | SM: 1-methyl-2-oxabicyclo[2.1.1]hexane-4-carboxylic acid |
| | LCMS m/z = 225 [M + H]⁺ |
| 178 | 4-chloro-2-(1-(fluoromethyl)-2-oxabicyclo[2.1.1]hexan- |
| | 4-yl)-6-methylpyrimidine |
| | 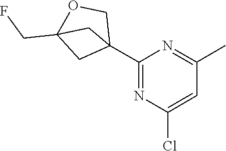 [SMILES: Cc1cc(Cl)nc(C23COC(CF)(C2)C3)n1] |
| | SM: 1-(fluoromethyl)-2-oxabicyclo[2.1.1]hexane-4- |
| | carboxylic acid. |
| | LCMS m/z = 243 [M + H]⁺ |
| 179 | 4-chloro-2-(1-fluorocyclopropyl)-6-methylpyrimidine |
| | 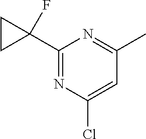 |
| | SM: 1-fluorocyclopropane-1-carboxylic acid. |
| | LCMS m/z = 187 [M + H]⁺ |
| 180 | 4-chloro-2-(1-(methoxymethyl)-2-oxabicyclo[2.1.1] |
| | hexan-4-yl)-6-methylpyrimidine |
| | 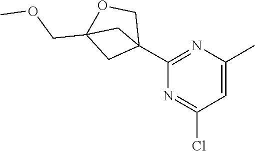 |
| | SM: 1-(methoxymethyl)-2-oxabicyclo[2.1.1]hexane-4- |
| | carboxylic acid. |
| | LCMS m/z = 255 [M + H]⁺ |

### Preparation 181

### 1,3-dioxoisoindolin-2-yl 2-oxabicyclo[2.1.1]hexane-4-carboxylate

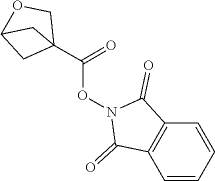 [SMILES: O=C1c2ccccc2C(=O)N1OC(=O)C12COC(C1)C2]

To a mixture of 2-oxabicyclo[2.1.1]hexane-4-carboxylic acid (1 g, 7.80 mmol), DMAP (19 mg, 0.156 mmol) and 2-hydroxyisoindoline-1,3-dione (1.27 g, 7.80 mmol) in DCM (50 mL) was added a solution of DCC (1.93 g, 9.37 mmol) in DCM (25 mL) and the mixture was stirred at rt overnight. The reaction mixture was filtered through Celite®, rinsed with ether, the filtrate was then passed through a short silica gel plug. The filtrate was concentrated in vacuo to give 1,3-dioxoisoindolin-2-yl 2-oxabicyclo[2.1.1]hexane-4-carboxylate (2.13 g, crude) as a white powder. ¹H NMR (CDCl₃, 400 MHz) δ 7.9-8.0 (m, 2H), 7.8-7.9 (m, 2H), 4.6-4.7 (m, 1H), 4.08 (s, 2H), 2.4-2.5 (m, 2H), 2.0-2.1 (m, 2H).

### Preparation 182

### 2-chloro-5-((difluoromethoxy)methyl)pyridine

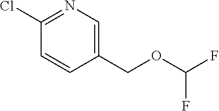 [SMILES: FC(F)OCc1ccc(Cl)nc1]

A mixture of (6-chloro-3-pyridyl)methanol (1 g, 6.97 mmol), CuI (265.3 mg, 1.39 mmol) and molecular sieves (0.5 g) in MeCN (8 mL) in a sealed tube was heated at 65° C. To this was added dropwise a solution of 2,2-difluoro-2-fluorosulfonyl-acetic acid (1.86 g, 10.45 mmol) in MeCN (2 mL) and the reaction stirred at 65° C. for 1 h. The cooled mixture was diluted with EtOAc, washed with NaHCO₃, the organic layer was dried and concentrated. The crude was purified by chromatography on silica gel (0-100% EtOAc/heptane) to give 2-chloro-5-((difluoromethoxy)methyl)pyridine (405 mg, 18% yield) as a yellow oil. LCMS m/z=194 [M+H]⁺

### Preparation 183

### 2-bromo-5-(1-fluoroethyl)pyridine

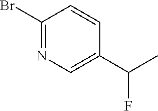

A mixture of 1-(6-bromo-3-pyridyl)ethanol (500 mg, 2.47 mmol) in DCM (10 mL) was cooled to −78° C. DAST (1.60 g, 9.90 mmol, 1.31 mL) was dropwise added, the solution stirred for 1 h at −78° C. then slowly warmed up to rt overnight. The reaction was quenched with water, the mixture stirred at rt for 15 min., washed with NaHCO₃ and the organic layer was dried and concentrated. The crude was purified by chromatography on silica gel (0-50% EtOAc/heptane) to give 2-bromo-5-(1-fluoroethyl)pyridine (228 mg, 45% yield). LCMS m/z=204, 206 [M+H]⁺

### Preparation 184

### 2-bromo-5-(1-methoxyethyl)pyridine

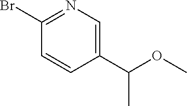

To a mixture of NaH (89 mg, 3.7 mmol) in THF (8 mL) at 0° C. was added dropwise, a solution of 1-(6-bromo-3-pyridyl)ethanol (500 mg, 2.47 mmol) in THF (2 mL) and the solution stirred at rt for 20 min. The solution was cooled to 0° C., iodomethane (154 μL, 2.47 mmol) was added dropwise and the reaction then stirred at rt for 1 h. The reaction was diluted with EtOAc, washed with brine, the organic layer was dried and concentrated. The crude was purified by chromatography on silica gel (0-80% EtOAc/heptane) to give 2-bromo-5-(1-methoxyethyl)pyridine (443 mg, 82% yield). LCMS m/z=216, 218 [M+H]+

### Preparation 185

### 2-bromo-6-(1,2-difluoroethyl)pyridine

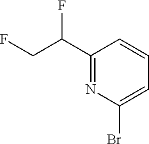 [SMILES: FCC(F)c1cccc(Br)n1]

To a solution of 1-(6-bromopyridin-2-yl)ethane-1,2-diol (476.0 mg, 2.64 mmol) in DCM (6 mL) was added DAST (1.5 mL, 11.35 mmol) and the reaction was stirred at 25° C. for 2 h. The mixture was quenched with H₂O (10 mL) and extracted with DCM (20 mL×3). The combined organic phase was washed with brine (10 mL×2), dried over Na₂SO₄ and filtered. The filtrate was concentrated under reduced pressure and the residue purified by chromatography on silica gel (PE/EtOAc=1/1) to give 2-bromo-6-(1,2-difluoroethyl)pyridine (440.0 mg, 75.0% yield) as a yellow oil. ¹H NMR (400 MHz, CDCl₃) δ ppm: 7.57-7.61 (m, 1H), 7.41-7.47 (m, 2H), 5.71-5.77 (m, 1H), 4.64-4.95 (m, 2H)

### Preparation 186

### 2-bromo-6-vinylpyrazine

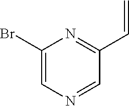

To a solution of 2,6-dibromopyrazine (3.8 g, 16.0 mmol), 4,4,5,5-tetramethyl-2-vinyl-1,3,2-dioxaborolane (2.5 g, 16.0 mmol) and K₂CO₃ (4.5 g, 31.9 mmol) in dioxane (30.0 mL) and H₂O (3.0 mL) was added Pd(dppf)Cl₂ (1.17 g, 1.60 mmol). The mixture was stirred at 70° C. for 3 h under N₂, then concentrated under reduced pressure. The crude was purified by column chromatography on silica gel (PE/EtOAc I/O to 10/1) to give 2-bromo-6-vinylpyrazine (2.0 g, 47.4% yield) as an oil. LCMS m/z=187.0 [M+H]⁺.

### Preparation 187

### 1-(6-bromopyrazin-2-yl)ethane-1,2-diol

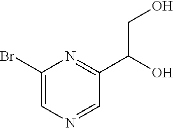 [SMILES: OCC(O)c1cncc(Br)n1]

To a solution of 2-bromo-6-vinylpyrazine (Preparation 186, 1.5 g, 5.68 mmol) in acetone (12 mL) and H₂O (4 mL) was added OsO₄ (250 mg, 0.984 mmol). NMO (997 mg, 8.51 mmol) was added and the reaction mixture was stirred at 25° C. for 2 h. The mixture was quenched with sat. Na₂SO₃ (100 mL) and filtered through Celite®, then extracted with EtOAc (3×50 mL) and concentrated under reduced pressure. The crude was purified by column chromatography on silica gel (PE/EtOAc I/O to 1/1) to give 1-(6-bromopyrazin-2-yl)ethane-1,2-diol (220 mg, 17.7% yield) as a yellow oil. LCMS m/z=221.1 [M+H]⁺.

### Preparation 188

### 2-bromo-6-(1,2-difluoroethyl)pyrazine

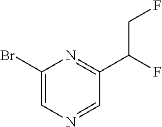 [SMILES: FCC(F)c1cncc(Br)n1]

2-Bromo-6-(1,2-difluoroethyl)pyrazine was obtained as a white oil, 150 mg, 67% from 1-(6-bromopyrazin-2-yl)ethane-1,2-diol (Preparation 187) and DAST following the procedure described in Preparation 185. LCMS m/z=223.0 [M+H]⁺.

### Preparation 189

### 2-chloro-4-methyl-6-vinylpyrimidine

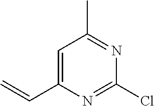

To a solution of 2,4-dichloro-6-methylpyrimidine (2.0 g, 12.3 mmol) in dioxane (20 mL) and H₂O (2 mL) was added 4,4,5,5-tetramethyl-2-vinyl-1,3,2-dioxaborolane (2.3 g, 14.7 mmol), Pd(dppf)Cl₂ (898 mg, 1.23 mmol) and K₃PO₄ (5.2 g, 24.5 mmol). The reaction mixture was stirred at 80° C. for 2 h under N₂, then concentrated and purified by prep-HPLC (Method N, Gradient 10-40%) to give 2-chloro-4-methyl-6-vinylpyrimidine (1.5 g, 79.1% yield) as a brown solid. LCMS m/z=155.1 [M+H]⁺.

### Preparation 190

### 1-(2-chloro-6-methylpyrimidin-4-yl)ethane-1,2-diol

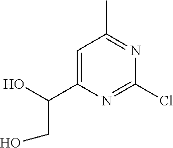

1-(2-Chloro-6-methylpyrimidin-4-yl)ethane-1,2-diol was obtained as a white solid, 200 mg, crude, from 1-(2-chloro-6-methylpyrimidin-4-yl)ethan-1-one (Preparation 189) and OsO₄ following the procedure described in Preparation 187. ¹H NMR: (500 MHz, CDCl₃) δ ppm: 7.32 (s, 1H), 3.96-4.00 (m, 1H), 3.80-3.83 (m, 1H), 3.68-3.70 (m, 1H), 2.54 (s, 3H).

### Preparation 191

### 2-chloro-4-(1,2-difluoroethyl)-6-methylpyrimidine

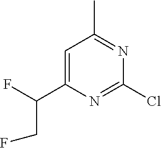

2-Chloro-4-(1,2-difluoroethyl)-6-methylpyrimidine was obtained as a yellow oil, 150 mg, 67.0% yield, from 1-(2-chloro-6-methylpyrimidin-4-yl)ethane-1,2-diol (Preparation 190) and DAST following the procedure described in Preparation 185. ¹H NMR: (500 MHz, CDCl₃) δ ppm: 7.40 (s, 1H), 5.64-5.70 (m, 1H), 4.81-5.00 (m, 2H), 2.61 (s, 3H).

### Preparation 192

### 4-chloro-2-iodo-6-methylpyrimidine

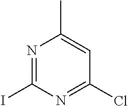

t-BuONO (7.2 g, 69.6 mmol) was added to a solution of 4-chloro-6-methylpyrimidin-2-amine (5.0 g, 34.8 mmol) and CH₂I₂ (10.3 g, 38.3 mmol) in MeCN (50.0 mL). The reaction mixture was stirred at 80° C. for 3.5 h, then concentrated under reduced pressure, treated with H₂O (30 mL) and extracted with EtOAc (3×35 mL). The organic phase was washed with brine (30 mL), dried with Na₂SO₄, filtered, concentrated, then purified by column chromatography on silica gel (PE/EtOAc 15/1 to 1/1) to give 4-chloro-2-iodo-6-methylpyrimidine (4.2 g, 47.4% yield) as a white solid. LCMS m/z=254.9 [M+H]⁺.

### Preparation 193

### 2-((tert-butyldimethylsilyl)oxy)-1-(4-chloro-6-methylpyrimidin-2-yl)ethan-1-ol

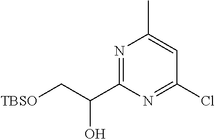 [SMILES: Cc1cc(Cl)nc(C(O)CO[Si](C)(C)C(C)(C)C)n1]

A solution of 4-chloro-2-iodo-6-methylpyrimidine (Preparation 192, 2.0 g, 7.86 mmol) in toluene (30 mL) was cooled to −70° C. under N₂. n-BuLi (2.5 M, 3.8 mL) was added dropwise over 30 min. After stirring at −70° C. for 20 min, 2-((tert-butyldimethylsilyl)oxy)acetaldehyde (1.6 g, 9.43 mmol) was added slowly and the reaction mixture stirred at −70° C. for 1 h. The mixture was quenched with MeOH (25 mL) and AcOH (1.6 mL), concentrated and extracted with EtOAc (3×20 mL). The combined organic layers were washed with brine (20 mL), dried over Na₂SO₄, filtered and concentrated under vacuum. The crude was purified by column chromatography on silica gel (PE/EtOAc 1/0 to 3/1) to give 2-((tert-butyldimethylsilyl)oxy)-1-(4-chloro-6-methylpyrimidin-2-yl)ethan-1-ol (890 mg, 37.4% yield) as yellow oil. LCMS m/z=303.2 [M+H]⁺.

### Preparation 194

### 1-(4-chloro-6-methylpyrimidin-2-yl)ethane-1,2-diol

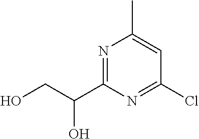

To a solution of 2-((tert-butyldimethylsilyl)oxy)-1-(4-chloro-6-methylpyrimidin-2-yl)ethan-1-ol (Preparation 193, 880 mg, 2.91 mmol) in THF (5.0 mL) was added TBAF (1 M, 8.8 mL) at 25° C. The reaction mixture was stirred for 2 h, then quenched with H₂O (20 mL) and extracted with EtOAc (3×20 mL). The combined organic layers were washed with H₂O (3×30 mL) and brine (3×30 mL), dried over Na₂SO₄, filtered and concentrated under reduced pressure. The crude was purified by column chromatography on silica gel (PE/EtOAc 10/1 to 1/1) to give 1-(4-chloro-6-methylpyrimidin-2-yl)ethane-1,2-diol (210 mg, 38.3% yield) as yellow oil. ¹H NMR: (400 MHz, CDCl₃) δ ppm: 7.15 (s, 1H), 4.80-4.86 (m, 1H), 3.97-4.04 (m, 2H), 2.53 (s, 3H).

### Preparation 195

### 4-chloro-2-(1,2-difluoroethyl)-6-methylpyrimidine

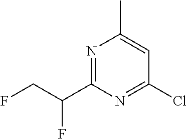

4-Chloro-2-(1,2-difluoroethyl)-6-methylpyrimidine was obtained as a yellow oil, 150 mg, 67.0% yield, from 1-(4-chloro-6-methylpyrimidin-2-yl)ethane-1,2-diol (Preparation 194) and DAST following the procedure described in Preparation 185. LCMS m/z=193.1 [M+H]⁺.

### Preparation 196

### 4-(benzyloxy)-6-methyl-2-(prop-1-en-2-yl)pyrimidine

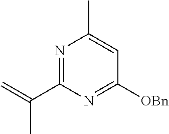 [SMILES: C=C(C)c1nc(C)cc(OCc2ccccc2)n1]

4-(Benzyloxy)-6-methyl-2-(prop-1-en-2-yl)pyrimidine was obtained as a colorless oil, 7.5 g, 65.4%, from 4-(benzyloxy)-2-chloro-6-methylpyrimidine (Preparation 134) and 4,4,5,5-tetramethyl-2-(prop-1-en-2-yl)-1,3,2-dioxaborolane, following a similar procedure to that described in Preparation 140, step 2). ¹H NMR: (400 MHz, CDCl₃) δ ppm: 7.44-7.46 (m, 2H), 7.32-7.39 (m, 3H), 6.46 (s, 1H), 6.39 (s, 1H), 5.48 (s, 1H), 5.45 (s, 2H), 2.44 (s, 3H), 2.22 (s, 3H).

### Preparation 197

### 2-(4-(benzyloxy)-6-methylpyrimidin-2-yl)propan-1-ol

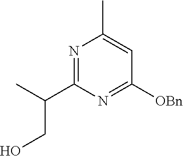 [SMILES: Cc1cc(OCc2ccccc2)nc(C(C)CO)n1]

To a solution of 4-(benzyloxy)-6-methyl-2-(prop-1-en-2-yl)pyrimidine (Preparation 196, 5.0 g, 20.8 mmol) in THF (100 mL) at 0° C. was added BH₃·THF (1 M, 41.6 mL). The reaction mixture was stirred at 25° C. for 3 h. After cooling to 0° C., the reaction mixture was quenched with water, then NaOH (4 M, 20.8 mL) and H₂O₂ (9.4 g, 83.2 mmol, 30% purity) were added. The solution was stirred at 60° C. for 16 h, quenched with aq. Na₂SO₃, treated with H₂O (150 mL) and extracted with DCM (3×100 mL). The combined organic phase was washed with brine (2×100 mL), dried over Na₂SO₄, filtered and concentrated under reduced pressure. The residue was purified by chromatography on silica gel (PE/EtOAc 20/1 to 1/1) to give 2-(4-(benzyloxy)-6-methylpyrimidin-2-yl)propan-1-ol (2.3 g, 42.8% yield) as colourless oil. ¹H NMR: (500 MHz, CDCl₃) δ ppm: 7.33-7.43 (m, 5H), 6.49 (s, 1H), 5.41 (s, 2H), 4.21 (brs, 1H), 3.91-3.94 (m, 1H), 3.80-3.82 (m, 1H), 3.11-3.15 (m, 1H), 2.42 (s, 3H), 1.33 (d, J=7.0 Hz, 3H).

### Preparation 198

### 4-(benzyloxy)-2-(1-fluoropropan-2-yl)-6-methylpyrimidine

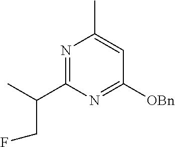 [SMILES: Cc1cc(OCc2ccccc2)nc(C(C)CF)n1]

4-(Benzyloxy)-2-(1-fluoropropan-2-yl)-6-methylpyrimidine was obtained as a colourless oil, 327 mg, 32.4% yield, from 2-(4-(benzyloxy)-6-methylpyrimidin-2-yl)propan-1-ol (Preparation 197) and DAST following the procedure described in Preparation 185. ¹H NMR (500 MHz, CDCl₃) δ ppm: 7.33-7.44 (m, 5H), 6.47 (s, 1H), 5.42 (s, 2H), 4.77-4.90 (m, 1H), 4.57-4.67 (m, 1H), 3.37-3.41 (m, 1H), 2.43 (s, 3H), 1.33 (d, J=7.0 Hz, 3H).

### Preparation 199

### 2-(1-fluoropropan-2-yl)-6-methylpyrimidin-4-ol

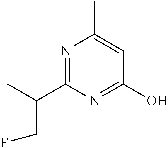

To a solution of 4-(benzyloxy)-2-(1-fluoropropan-2-yl)-6-methylpyrimidine (Preparation 198, 327 mg, 1.26 mmol) in THF (3.0 mL) was added Pd/C (134 mg, 0.126 mmol, 10% purity). The reaction mixture was stirred at 25° C. for 2 h, then filtered and concentrated under reduced pressure to give 2-(1-fluoropropan-2-yl)-6-methylpyrimidin-4-ol (197 mg, 92.0% yield) as a white solid. ¹H NMR: (400 MHz, CDCl₃) δ ppm: 12.87 (br s, 1H), 6.20 (s, 1H), 4.53-4.80 (m, 2H), 3.18-3.24 (m, 1H), 2.31 (s, 3H), 1.34 (d, J=7.2 Hz, 3H).

### Preparation 200

### 4-chloro-2-(1-fluoropropan-2-yl)-6-methylpyrimidine

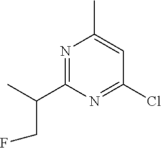

A solution of 2-(1-fluoropropan-2-yl)-6-methylpyrimidin-4-ol (Preparation 199, 197 mg, 1.15 mmol) in POCl₃ (1.7 g, 10.73 mmol, 1 mL) was stirred at 100° C. for 2 h. The mixture was concentrated under reduced pressure and the residue was purified by chromatography on silica gel (PE/EtOAc 20/1 to 0/1) to give 4-chloro-2-(1-fluoropropan-2-yl)-6-methylpyrimidine (127 mg, 58.4% yield) as colorless oil. LCMS m/z=189.1 [M+H]⁺.

### Preparation 201

### 2-(4-(benzyloxy)-6-methylpyrimidin-2-yl)propane-1,2-diol

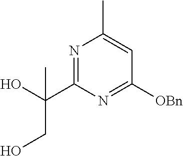 [SMILES: Cc1cc(OCc2ccccc2)nc(C(C)(O)CO)n1]

2-(4-(Benzyloxy)-6-methylpyrimidin-2-yl)propane-1,2-diol was obtained as a yellow oil, 670 mg, 58.7% yield, from 4-(benzyloxy)-6-methyl-2-(prop-1-en-2-yl)pyrimidine (Preparation 196) and OsO₄ following the procedure described in Preparation 187. ¹H NMR: (500 MHz, CDCl₃) δ ppm: 7.32-7.43 (m, 5H), 6.52 (s, 1H), 5.38-5.44 (m, 2H), 5.05 (s, 1H), 3.91 (d, J=10.5 Hz, 1H), 3.73 (d, J=11.0 Hz, 1H), 2.70 (br s, 1H), 2.44 (s, 3H), 1.48 (s, 3H).

### Preparation 202

### 4-(benzyloxy)-2-(1,2-difluoropropan-2-yl)-6-methylpyrimidine

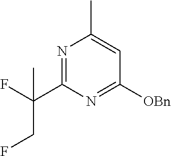 [SMILES: Cc1cc(OCc2ccccc2)nc(C(C)(F)CF)n1]

4-(Benzyloxy)-2-(1,2-difluoropropan-2-yl)-6-methylpyrimidine was obtained as a yellow oil, 220 mg, 35.0% yield, from 2-(4-(benzyloxy)-6-methylpyrimidin-2-yl)propane-1,2-diol (Preparation 201) and DAST following the procedure described in Preparation 188. ¹H NMR: (400 MHz, CDCl₃) δ ppm: 7.35-7.44 (m, 5H), 6.56 (s, 1H), 5.41 (s, 2H), 4.70-4.96 (m, 2H), 2.47 (s, 3H), 1.71-1.77 (m, 3H).

### Preparation 203

### 2-(1,2-difluoropropan-2-yl)-6-methylpyrimidin-4-ol

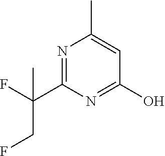

A solution of 4-(benzyloxy)-2-(1,2-difluoropropan-2-yl)-6-methylpyrimidine (Preparation 202, 200 mg, 0.719 mmol) and TFA (4.47 g, 39.2 mmol, 3 mL) was stirred at 100° C. for 3 h. The mixture was concentrated and the residue was purified by chromatography on silica gel (PE/EtOAc 10/1) to give 2-(1,2-difluoropropan-2-yl)-6-methylpyrimidin-4-ol (100 mg, 74.0% yield) as a yellow solid. ¹H NMR: (400 MHz, CDCl₃) δ ppm: 6.22 (s, 1H), 4.61-4.81 (m, 2H), 2.29 (s, 3H), 1.68-1.73 (m, 3H).

### Preparation 204

### 4-chloro-2-(1,2-difluoropropan-2-yl)-6-methylpyrimidine

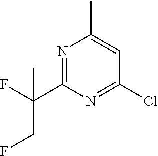

A solution of 2-(1,2-difluoropropan-2-yl)-6-methylpyrimidin-4-ol (Preparation 203, 90 mg, 0.473 mmol) and POCl₃ (3.3 g, 21.5 mmol) was stirred at 100° C. for 3 h, then quenched with H₂O (10 mL) and extracted with EtOAc (2×10 mL). The combined organic phase was washed with brine (2×10 mL), dried over Na₂SO₄, filtered and concentrated under reduced pressure. The residue was purified by chromatography on silica gel (EtOAc) to give 4-chloro-2-(1,2-difluoropropan-2-yl)-6-methylpyrimidine (100 mg) as a yellow oil. ¹H NMR: (500 MHz, CDCl₃) δ ppm: 7.23 (s, 1H), 4.73-4.97 (m, 2H), 2.10 (s, 3H), 1.75-1.80 (m, 3H).

### Preparation 205

### 2-(4-(benzyloxy)pyrimidin-2-yl)propan-2-ol

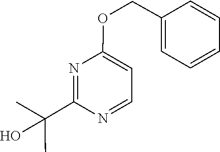 [SMILES: CC(C)(O)c1nccc(OCc2ccccc2)n1]

To a solution of methyl 4-(benzyloxy)pyrimidine-2-carboxylate (Preparation 144, 1.5 g, 6.14 mmol) in THF (20.0 mL) was added dropwise CH₃MgBr (3 M, 2.05 mL) at 0° C. The mixture was stirred for 1 h, then warmed 25° C. for 12 h under N₂. The mixture was quenched with NH₄Cl (sat. 20 mL), poured into H₂O (30 mL) and extracted with EtOAc (3×30 mL), the combined organic layer was dried over anhydrous Na₂SO₄, filtered and concentrated. The residue was purified by chromatography (PE/EtOAc=3/1) on silica gel to give 2-(4-(benzyloxy)pyrimidin-2-yl)propan-2-ol (358.2 mg, 23.9% yield) as a white solid. ¹H NMR: (400 MHz, CDCl₃) δ ppm: 8.40 (d, J=5.6 Hz, 1H), 7.34-7.46 (m, 5H), 6.65 (d, J=5.6 Hz, 1H), 5.44 (s, 2H), 4.68 (s, 1H), 1.56 (s, 6H).

### Preparation 206

### 4-(benzyloxy)-2-(2-fluoropropan-2-yl)pyrimidine

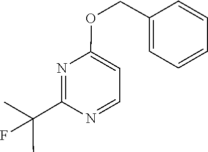 [SMILES: CC(C)(F)c1nccc(OCc2ccccc2)n1]

4-(benzyloxy)-2-(2-fluoropropan-2-yl)pyrimidine was obtained as a yellow oil, 556.4 mg, 78.8%, from 2-(4-(benzyloxy)pyrimidin-2-yl)propan-2-ol, following the procedure described in Preparation 130. 1H NMR (500 MHz, CDCl₃) δ ppm: 8.46-8.48 (m, 1H), 7.33-7.46 (m, 5H), 6.67 (d, J=5.5 Hz, 1H), 5.45 (s, 2H), 1.79 (s, 3H), 1.74 (s, 3H).

### Preparation 207

### 4-chloro-2-(2-fluoropropan-2-yl)pyrimidine

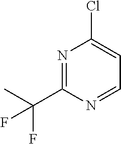

To a solution of 4-(benzyloxy)-2-(2-fluoropropan-2-yl)pyrimidine (Preparation 206, 556.4 mg, 2.26 mmol) in TFA (4.47 g, 39.2 mmol) was stirred at 100° C. for 12 h. The mixture was evaporated under reduced pressure to give 2-(2-fluoropropan-2-yl)pyrimidin-4-ol as a white solid.

A solution of 2-(2-fluoropropan-2-yl)pyrimidin-4-ol (100.0 mg, 0.640 mmol) in POCl₃ (2.0 mL) was stirred at 100° C. for 2 h. The mixture was concentrated, diluted with H₂O (10 mL) and extracted with EtOAc (3×10 mL). The combined organic phase was dried over anhydrous Na₂SO₄, filtered and concentrated to give 4-chloro-2-(2-fluoropropan-2-yl)pyrimidine (82.0 mg, 73.34% yield) as colorless oil. ¹H NMR (500 MHz, CDCl₃) δ ppm: 8.66 (d, J=5.5 Hz, 1H), 7.29 (d, J=5.5 Hz, 1H), 1.81 (s, 3H), 1.77 (s, 3H).

### Preparations 208 to 219

The compounds in the following table were prepared from 2-bromo-5-fluoroisonicotinic acid and the appropriate alcohol (SM), following a similar procedure to that described in Preparation 14.

| Preparation | |
|---|---|
| No | Name, Structure, Starting Material (SM), Data |
| 208 | 2-bromo-5-((1-(trifluoromethyl)cyclopropyl)methoxy) |
| | isonicotinic acid |
| | 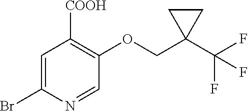 [SMILES: O=C(O)c1cc(Br)ncc1OCC1(C(F)(F)F)CC1] |
| | SM: (1-(trifluoromethyl)cyclopropyl)methanol |
| | 646 mg, 83.6% yield as a yellow solid. |
| | LCMS m/z = 342.0 [M + H]⁺ |
| 209 | 2-bromo-5-((2,2-difluorocyclopropyl)methoxy)isonicotinic |
| | acid |
| | 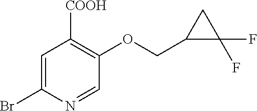 [SMILES: O=C(O)c1cc(Br)ncc1OCC1CC1(F)F] |
| | SM: 2,2-difluorocyclopropyl)methanol |
| | 1.05 g, 75.0% yield as a white solid. LCMS m/z = |
| | 309.8 [M + H]⁺ |
| 210 | 2-bromo-5-((1-methylcyclopropyl)methoxy)isonicotinic acid |
| | 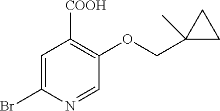 [SMILES: CC1(COc2cnc(Br)cc2C(=O)O)CC1] |
| | SM: (1-methylcyclopropyl)methanol |
| | 1.05 g, 96.1% yield as a white solid. ¹H NMR: |
| | (400 MHz, CDCl₃) δ ppm: 10.66 (br s, 1H), 8.23 (s, 1H), |
| | 8.14 (s, 1H), 4.11 (s, 2H), 1.29 (s, 3H), 0.59-0.67 (m, |
| | 4H). |
| 211 | 2-bromo-5-(1-cyclopropylethoxy)isonicotinic acid |
| | 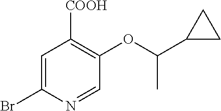 [SMILES: CC(Oc1cnc(Br)cc1C(=O)O)C1CC1] |
| | SM: 1-cyclopropylethan-1-ol |
| | 1.25 g, 32.0% yield as a white solid. ¹H NMR: (400 |
| | MHz, CDCl₃) δ ppm: 8.27 (s, 1H), 8.13 (s, |
| | 1H), 4.14-4.21 (m, 1H), 1.56 (d, J = 6.0 Hz, 3H), 0.84-0.85 |
| | (m, 1H), 0.68-0.75 (m, 2H), 0.39-0.41 (m, 2H). |
| 212 | 2-bromo-5-((1-cyanocyclopropyl)methoxy)isonicotinic acid |
| | 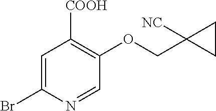 [SMILES: N#CC1(COc2cnc(Br)cc2C(=O)O)CC1] |
| | SM: 1-(hydroxymethyl)cyclopropane-1-carbonitrile |
| | 800 mg, 59.2% yield as a yellow solid. LCMS m/z = |
| | 298.8 [M + H]⁺ |
| 213 | 2-bromo-5-((1-methoxypropan-2-yl)oxy)isonicotinic acid |
| | 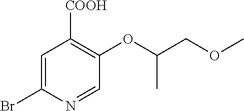 [SMILES: COCC(C)Oc1cnc(Br)cc1C(=O)O] |
| | SM: 1-methoxypropan-2-ol |
| | 780 mg, 59.1% yield as a yellow solid. LCMS m/z = |
| | 292.0 [M + H]⁺ |
| 214 | 2-bromo-5-(2-methoxypropoxy)isonicotinic acid |
| | 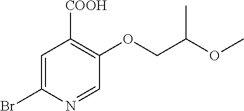 [SMILES: COC(C)COc1cnc(Br)cc1C(=O)O] |
| | SM: 2-methoxypropan-1-ol |
| | 1.5 g, crude, as yellow solid. LCMS m/z = 291.9 [M + H]⁺ |
| 215 | 2-bromo-5-(2-(pyrrolidin-1-yl)ethoxy)isonicotinic acid |
| | 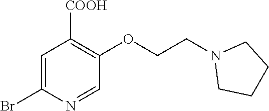 [SMILES: O=C(O)c1cc(Br)ncc1OCCN1CCCC1] |
| | SM: 2-(pyrrolidin-1-yl)ethan-1-ol |
| | 1.3 g, 45.4% yield as a white solid. LCMS m/z = |
| | 315.0 [M + H]⁺ |
| 216 | 2-bromo-5-(oxetan-3-ylmethoxy)isonicotinic acid |
| | 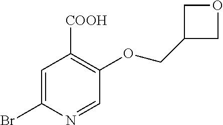 [SMILES: O=C(O)c1cc(Br)ncc1OCC1COC1] |
| | SM: oxetan-3-ylmethanol |
| | 680 mg, 80.0% yield as white solid. ¹H NMR: (400 MHz, |
| | DMSO-d₆) δ ppm: 8.39 (s, 1H), 7.71 (s, 1H), 4.65- |
| | 4.68 (m, 2H), 4.44 (t, J-6.4 Hz, 2H), 4.37 (d, J = 6.4 Hz, |
| | 2H), 3.39-3.43 (m, 1H). |
| 217 | 2-bromo-5-(oxetan-2-ylmethoxy)isonicotinic acid |
| | 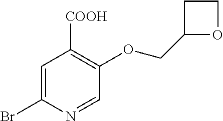 [SMILES: O=C(O)c1cc(Br)ncc1OCC1CCO1] |
| | SM: oxetan-2-ylmethanol |
| | 1.7 g, 86.5% yield as a white solid. ¹H NMR: (400 MHz, |
| | DMSO-d₆) δ ppm: 8.35 (s, 1H), 7.66 (s, 1H), 4.98-4.99 |
| | (m, 1H), 4.46-4.50 (m, 2H), 4.27-4.30 (m, 2H), |
| | 2.61-2.70 (m, 2H). |
| 218 | 2-bromo-5-(oxetan-3-yloxy)isonicotinic acid |
| | 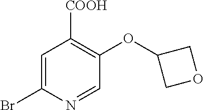 [SMILES: O=C(O)c1cc(Br)ncc1OC1COC1] |
| | SM: oxetan-3-ol |
| | 1.2 g, 96.3% yield as a white solid. LCMS m/z = |
| | 276.0 [M + H]⁺ |
| 219 | 2-bromo-5-(cyclobutylmethoxy)isonicotinic acid |
| | 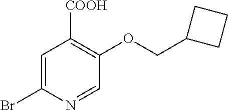 [SMILES: O=C(O)c1cc(Br)ncc1OCC1CCC1] |
| | SM: cyclobutylmethanol |
| | 1.1 g, 83.8% yield as yellow solid. ¹H NMR: (400 MHz, |
| | DMSO-d₆) δ ppm: 8.29 (s, 1H), 8.12 (s, 1H), 4.31 (d, |
| | J = 7.2 Hz, 2H), 2.90-2.95 (m, 1H), 2.21-2.25 (m, |
| | 2H), 1.90-2.06 (m, 4H). |

### Preparations 220 to 231

The compounds in the following table were obtained from the appropriate 2-bromoisonicotinic acid and DPPA, following a similar reaction to that described in Preparation 22.

| Preparation | |
|---|---|
| No | Name, Structure, Starting Material (SM), Data |
| 220 | tert-butyl (2-bromo-5-((1-(trifluoromethyl)cyclopropyl) |
| | methoxy)pyridin-4-yl)carbamate |
| | 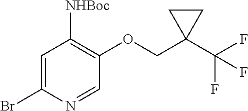 [SMILES: CC(C)(C)OC(=O)Nc1cc(Br)ncc1OCC1(C(F)(F)F)CC1] |
| | SM: 2-bromo-5-((1-(trifluoromethyl)cyclopropyl) |
| | methoxy)isonicotinic acid |
| | (Preparation 208) |
| | 530 mg, 70.7% yield as a white solid. ¹H NMR: (400 MHz, |
| | CDCl₃) δ ppm: 8.25 (s, 1H), 7.78 (s, 1H), 7.22 |
| | (br s, 1H), 4.12 (s, 2H), 1.53 (s, 9H), 1.23- |
| | 1.26 (m, 2H), 0.91-0.94 (m, 2H). |
| 221 | tert-butyl (2-bromo-5-((2,2-difluorocyclopropyl) |
| | methoxy)pyridin-4-yl)carbamate |
| | 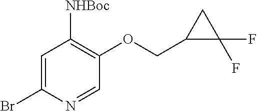 [SMILES: CC(C)(C)OC(=O)Nc1cc(Br)ncc1OCC1CC1(F)F] |
| | SM: 2-bromo-5-((2,2-difluorocyclopropyl)methoxy) |
| | isonicotinic acid |
| | (Preparation 209) |
| | 800 mg, 65.0% yield as a yellow solid. ¹H NMR: |
| | (500 MHz, CDCl₃) δ ppm: |
| | 8.27 (s, 1H), 7.84 (s, 1H), 7.15 (s, 1H), 4.10-4.21 |
| | (m, 2H), 2.09-2.13 (m, 1H), |
| | 1.65-1.68 (m, 1H), 1.55 (s, 9H), 1.30-1.34 (m, 1H). |
| 222 | tert-butyl (2-bromo-5-((1-methylcyclopropyl)methoxy) |
| | pyridin-4-yl)carbamate |
| | 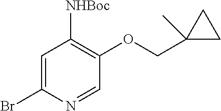 [SMILES: CC1(COc2cnc(Br)cc2NC(=O)OC(C)(C)C)CC1] |
| | SM: 2-bromo-5-((1-methylcyclopropyl)methoxy) |
| | isonicotinic acid |
| | (Preparation 210) |
| | 988 mg, 79.1% yield as a white solid. ¹H NMR: (500 MHz, |
| | CDCl₃) δ ppm: |
| | 8.22 (s, 1H), 7.78 (s, 1H), 7.20 (s, 1H), 3.83 (s, 2H), 1.55 |
| | (s, 9H), 1.25 (s, 3H), |
| | 0.55-0.58 (m, 2H), 0.49-0.52 (m, 2H). |
| 223 | tert-butyl (2-bromo-5-(1-cyclopropylethoxy)pyridin- |
| | 4-yl)carbamate |
| | 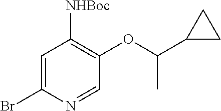 [SMILES: CC(Oc1cnc(Br)cc1NC(=O)OC(C)(C)C)C1CC1] |
| | SM: 2-bromo-5-(1-cyclopropylethoxy)isonicotinic acid |
| | (Preparation 211) |
| | 300 mg, 20% yield as a white solid. ¹H NMR: (400 |
| | MHz, CDCl₃) δ ppm: 8.23 (s, 1H), 7.84 (s, 1H), 3.75-3.84 |
| | (m, 1H), 1.55 (s, 9H), 1.42 (d, J = 6.4 Hz, 3H), |
| | 1.10-1.19 (m, 1H), 0.60-0.66 (m, 2H), 0.28-0.37 (m, 2H). |
| 224 | tert-butyl (2-bromo-5-((1-cyanocyclopropyl)methoxy) |
| | pyridin-4-yl)carbamate |
| | 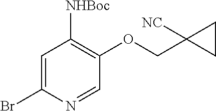 [SMILES: CC(C)(C)OC(=O)Nc1cc(Br)ncc1OCC1(C#N)CC1] |
| | SM: 2-bromo-5-((1-cyanocyclopropyl)methoxy)isonicotinic |
| | acid (Preparation 212) |
| | 600 mg, 60.5% yield as a yellow solid. ¹H NMR: (400 MHz, |
| | CDCl₃) δ ppm: 8.31 (s, 1H), 7.78 (s, 1H), 4.02 (s, 2H), |
| | 1.56 (s, 9H), 1.50-1.53 (m, 2H), 1.13- |
| | 1.17 (m, 2H). |
| 225 | tert-butyl (2-bromo-5-((1-methoxypropan-2-yl)oxy) |
| | pyridin-4-yl)carbamate |
| | 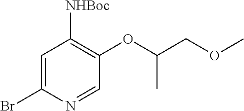 [SMILES: COCC(C)Oc1cnc(Br)cc1NC(=O)OC(C)(C)C] |
| | SM: 2-bromo-5-((1-methoxypropan-2-yl)oxy)isonicotinic |
| | acid (Preparation 213) |
| | 503 mg, 52.5% yield as a white solid. LCMS m/z = |
| | 362.9 [M + H]⁺ |
| 226 | tert-butyl (2-bromo-5-(2-methoxypropoxy)pyridin- |
| | 4-yl)carbamate |
| | 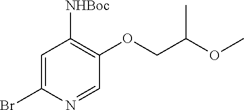 [SMILES: COC(C)COc1cnc(Br)cc1NC(=O)OC(C)(C)C] |
| | SM: 2-bromo-5-(2-methoxypropoxy)isonicotinic acid |
| | (Preparation 214) |
| | 850 mg, 45.5% yield as a yellow solid. LCMS m/z = |
| | 362.8 [M + H]⁺ |
| 227 | tert-butyl (2-bromo-5-(2-(pyrrolidin-1-yl)ethoxy)pyridin-4-yl) |
| | carbamate |
| | 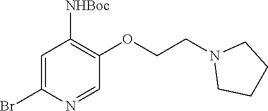 [SMILES: CC(C)(C)OC(=O)Nc1cc(Br)ncc1OCCN1CCCC1] |
| | SM: 2-bromo-5-(2-(pyrrolidin-1-yl)ethoxy)isonicotinic acid |
| | (Preparation 215) |
| | 80.0 mg, 32.6% yield as a white solid. LCMS m/z = |
| | 388.1 [M + H]⁺ |
| 228 | tert-butyl (2-bromo-5-(oxetan-3-ylmethoxy)pyridin-4-yl) |
| | carbamate |
| | 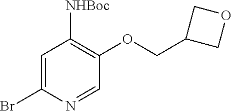 [SMILES: CC(C)(C)OC(=O)Nc1cc(Br)ncc1OCC1COC1] |
| | SM: 2-bromo-5-(oxetan-3-ylmethoxy)isonicotinic acid |
| | (Preparation 216) |
| | 580 mg, 72.5% yield as a white solid. ¹H NMR: (400 MHz, |
| | CDCl₃) δ ppm: 8.26 (s, 1H), 7.89 (s, 1H), 7.10 (s, 1H), |
| | 4.92-4.95 (m, 2H), 4.55 (t, J-6.0 Hz, 2H), 4.34 (d, J = |
| | 6.8 Hz, 2H), 3.40-3.55 (m, 1H), 1.54 (s, 9H) |
| 229 | tert-butyl (2-bromo-5-(oxetan-2-ylmethoxy)pyridin-4- |
| | yl)carbamate |
| | 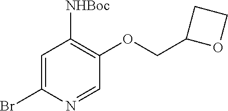 [SMILES: CC(C)(C)OC(=O)Nc1cc(Br)ncc1OCC1CCO1] |
| | SM: 2-bromo-5-(oxetan-2-ylmethoxy)isonicotinic acid |
| | (Preparation 217) |
| | 1.3 g, 61.3% yield as a white solid. ¹H NMR: (400 MHz, |
| | CDCl₃) δ ppm: 8.27 (s, 1H), 7.93 (s, 1H), 7.64 |
| | (s, 1H), 5.17-5.19 (m, 1H), 4.66-4.79 (m, 1H), |
| | 4.64-4.65 (m, 1H), 4.26-4.31 (m, 1H), 4.09- |
| | 4.12 (m, 1H), 2.80-2.85 (m, 1H), |
| | 2.63-2.68 (m, 1H), 1.52 (s, 9H). |
| 230 | tert-butyl (2-bromo-5-(oxetan-3-yloxy)pyridin-4-yl) |
| | carbamate |
| | 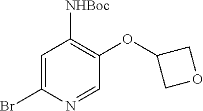 [SMILES: CC(C)(C)OC(=O)Nc1cc(Br)ncc1OC1COC1] |
| | SM: 2-bromo-5-(oxetan-3-yloxy)isonicotinic acid |
| | (Preparation 218) |
| | 666 mg, 52.9% yield as a white solid. LCMS m/z = |
| | 346.9 [M + H]⁺ |
| 231 | tert-butyl (2-bromo-5-(cyclobutylmethoxy)pyridin-4-yl) |
| | carbamate |
| | 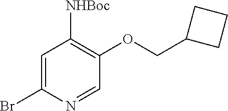 [SMILES: CC(C)(C)OC(=O)Nc1cc(Br)ncc1OCC1CCC1] |
| | SM: 2-bromo-5-(cyclobutylmethoxy)isonicotinic acid |
| | (Preparation 219) |
| | 900 mg, 72.1% yield as a yellow solid. ¹H NMR: (400 MHz, |
| | CDCl₃) δ ppm: 8.21 (s, 1H), 7.83 (s, 1H), 7.13 (s, 1H), |
| | 4.03-4.05 (m, 2H), 2.81-2.87 (m, 1H), |
| | 2.16-2.19 (m, 2H), 1.85-2.01 (m, 4H), 1.53 (s, 9H). |

### Preparation 232 to 240

The compounds in the following table were prepared from the appropriate 2-bromo or 2-chloropyridine and acetamide, following a similar procedure to that described in Preparation 32.

| Preparation | |
|---|---|
| No | Name, Structure, Starting Material (SM), Data |
| 232 | tert-butyl (2-acetamido-5-((1- |
| | (trifluoromethyl)cyclopropyl)methoxy)pyridin-4-yl)carbamate |
| | 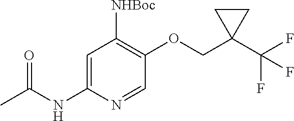 [SMILES: CC(=O)Nc1cc(NC(=O)OC(C)(C)C)c(OCC2(C(F)(F)F)CC2)cn1] |
| | SM: tert-butyl (2-bromo-5-((1- |
| | (trifluoromethyl)cyclopropyl)methoxy)pyridin-4-yl)carbamate |
| | (Preparation 220) |
| | 377 mg, 76.6% yield as a light yellow solid. ¹H NMR: (500 |
| | MHz, CDCl₃) δ ppm: 8.86 (br s, 1H), 7.75 (s, 1H), 7.70 (s, |
| | 1H), 7.19 (s, 1H), 4.10 (s, 2H),2.16 (s, 3H), 1.55 (s, 9H), |
| | 1.20-1.23 (m, 2H), 0.89-0.92 (m, 2H). |
| 233 | tert-butyl (2-acetamido-5-((2,2-difluorocyclopropyl)methoxy) |
| | pyridin-4-yl)carbamate |
| | 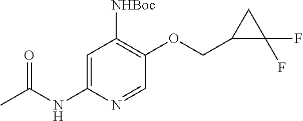 [SMILES: CC(=O)Nc1cc(NC(=O)OC(C)(C)C)c(OCC2CC2(F)F)cn1] |
| | SM: tert-butyl (2-bromo-5-((2,2-difluorocyclopropyl) |
| | methoxy)pyridin-4-yl)carbamate (Preparation 221) |
| | 560 mg, 74.3% yield as a yellow solid. LCMS m/z = |
| | 358.0 [M + H]⁺ |
| 234 | tert-butyl (2-acetamido-5-((1-methylcyclopropyl) |
| | methoxy)pyridin-4-yl)carbamate |
| | 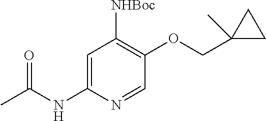 [SMILES: CC(=O)Nc1cc(NC(=O)OC(C)(C)C)c(OCC2(C)CC2)cn1] |
| | SM: tert-butyl (2-bromo-5-((1-methylcyclopropyl) |
| | methoxy)pyridin-4-yl)carbamate |
| | (Preparation 222) |
| | 704 mg, 76.7% yield as a yellow solid. ¹H NMR: (400 |
| | MHz, CDCl₃) δ ppm: 8.82 (s, 1H), 7.91-8.05 (m, 1H), |
| | 7.69 (s, 1H), 7.19 (s, 1H), 3.80 (s, 2H), 2.16 (s, 3H), |
| | 1.56 (s, 9H), 1.25 (s, 3H), 0.52-0.55 (m, 2H), 0.46-0.49 |
| | (m, 2H). |
| 235 | tert-butyl (2-acetamido-5-(1-cyclopropylethoxy)pyridin- |
| | 4-yl)carbamate |
| | 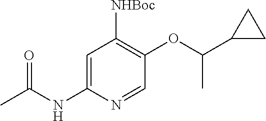 [SMILES: CC(=O)Nc1cc(NC(=O)OC(C)(C)C)c(OC(C)C2CC2)cn1] |
| | SM: tert-butyl (2-bromo-5-(1-cyclopropylethoxy)pyridin- |
| | 4-yl)carbamate |
| | (Preparation 223) |
| | 162 mg, 61.5% yield as a yellow solid. ¹H NMR: (400 |
| | MHz, CDCl₃) δ ppm: 8.82 (s, 1H), 7.76 (s, 2H), 7.24 |
| | (s, 1H), 3.56-3.78 (m, 1H), 2.16 (s, 3H), 1.56 (s, 9H), 1.41 |
| | (d, J = 6.4 Hz, 3H), 1.05-1.18 (m, 1H), 0.54-0.64 (m, |
| | 2H), 0.21-0.37 (m, 2H). |
| 236 | tert-butyl (2-acetamido-5-((1-cyanocyclopropyl)methoxy) |
| | pyridin-4-yl)carbamate |
| | 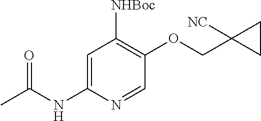 [SMILES: CC(=O)Nc1cc(NC(=O)OC(C)(C)C)c(OCC2(C#N)CC2)cn1] |
| | SM: tert-butyl (2-bromo-5-((1-cyanocyclopropyl)methoxy) |
| | pyridin-4-yl)carbamate |
| | (Preparation 224)) |
| | 180 mg, 33.0% yield as a yellow solid. ¹H NMR: (500 |
| | MHz, CDCl₃) δ ppm: 8.89 (s, 1H), 8.44 (s, 1H), 7.67 (s, |
| | 1H), 7.22 (s, 1H), 4.00 (s, 2H), 2.19 (s, 3H), |
| | 1.57 (s, 9H), 1.49-1.50 (m, 2H), 1.12-1.14 (m, 2H). |
| 237 | tert-butyl (2-acetamido-5-((1-methoxypropan-2-yl)oxy) |
| | pyridin-4-yl)carbamate |
| | 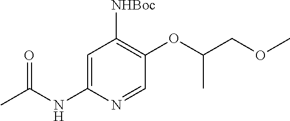 [SMILES: COCC(C)Oc1cnc(NC(C)=O)cc1NC(=O)OC(C)(C)C] |
| | SM: tert-butyl (2-bromo-5-((1-methoxypropan-2-yl)oxy) |
| | pyridin-4-yl)carbamate |
| | (Preparation 225) |
| | 410 mg, 90.9% yield as a white solid. LCMS m/z = |
| | 340.1 [M + H]⁺ |
| 238 | tert-butyl (2-acetamido-5-(2-methoxypropoxy)pyridin- |
| | 4-yl)carbamate |
| | 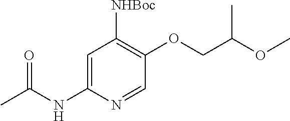 [SMILES: COC(C)COc1cnc(NC(C)=O)cc1NC(=O)OC(C)(C)C] |
| | SM: tert-butyl (2-bromo-5-(2-methoxypropoxy)pyridin-4- |
| | yl)carbamate |
| | (Preparation 226) |
| | 400 mg, 50.1% yield as a white solid. LCMS m/z = |
| | 340.2 [M + H]⁺ |
| 239 | tert-butyl (2-acetamido-5-(2-(pyrrolidin-1-yl)ethoxy) |
| | pyridin-4-yl)carbamate |
| | 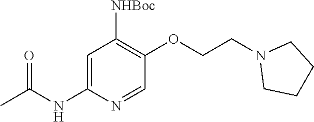 [SMILES: CC(=O)Nc1cc(NC(=O)OC(C)(C)C)c(OCCN2CCCC2)cn1] |
| | SM: tert-butyl (2-bromo-5-(2-(pyrrolidin-1-yl)ethoxy) |
| | pyridin-4-yl)carbamate |
| | (Preparation 227) |
| | 150 mg, 33.1% yield as a white solid. LCMS m/z = |
| | 365.2 [M + H]⁺ |
| 240 | tert-butyl (2-acetamido-5-(cyclobutylmethoxy) |
| | pyridin-4-yl)carbamate |
| | 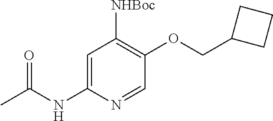 [SMILES: CC(=O)Nc1cc(NC(=O)OC(C)(C)C)c(OCC2CCC2)cn1] |
| | SM: tert-butyl (2-bromo-5-(cyclobutylmethoxy) |
| | pyridin-4-yl)carbamate |
| | (Preparation 231) |
| | 689 mg, 85.3% yield as a yellow solid. ¹H NMR: (400 |
| | MHz, CDCl₃) δ ppm : 8.82 (br s, 1 H), 8.00 (br s, 1H), |
| | 7.73 (s, 1H), 7.13 (s, 1H), 4.01 (d, J = 6.8 Hz, 2H), |
| | 2.74-2.83 (m, 1H) 2.14-2.17 (m, 4H), 1.83-2.04 |
| | (m, 5H), 1.55 (s, 9H). |

### Preparation 241

### tert-butyl methyl (5-(2-methoxyethoxy)pyridine-2,4-diyl)dicarbamate

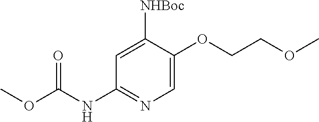

tert-Butyl methyl (5-(2-methoxyethoxy)pyridine-2,4-diyl)dicarbamate was obtained as a brwon oil, 570 mg, 58%, from tert-butyl (2-bromo-5-(2-methoxyethoxy)pyridin-4-yl)carbamate

(Preparation 22) and methyl carbamate, following the procedure described in Preparation 32. ¹H NMR: (400 MHz, CDCl₃) δ ppm: 8.78 (s, 1H), 8.02 (s, 1H), 7.89 (s, 1H), 4.12-4.14 (m, 2H), 3.79 (s, 3H), 3.68-3.71 (m, 2H), 3.47 (s, 3H), 1.53 (s, 9H).

### Preparation 242

### tert-butyl (5-(2-methoxyethoxy)-2-propionamidopyridin-4-yl)carbamate

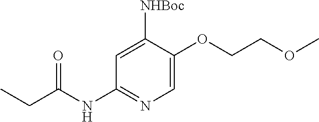 [SMILES: CCC(=O)Nc1cc(NC(=O)OC(C)(C)C)c(OCCOC)cn1]

tert-Butyl (5-(2-methoxyethoxy)-2-propionamidopyridin-4-yl)carbamate was obtained, 950 mg, 97.2%, as a brown solid, from tert-butyl (2-bromo-5-(2-methoxyethoxy)pyridin-4-yl)carbamate (Preparation 22) and propionamide, following the procedure described in Preparation 32. ¹H NMR: (400 MHz, CDCl₃) δ ppm: 8.86 (s, 1H), 7.88 (s, 1H), 7.75 (s, 2H), 4.12-4.15 (m, 2H), 3.68-3.70 (m, 2H), 3.48 (s, 3H), 2.21-2.37 (m, 2H), 1.54 (s, 9H), 1.20-1.24 (m, 3H).

### Preparation 243

### tert-butyl methyl (5-(2-(dimethylamino)ethoxy)pyridine-2,4-diyl)dicarbamate

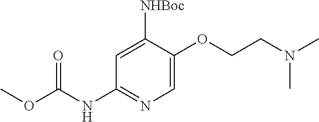

A mixture of (Preparation 30, 250 mg, 0.694 mmol), methyl carbamate (250 mg, 3.33 mmol), Cs₂CO₃ (500 mg, 1.53 mmol), BrettPhos Pd G3 (25 mg, 27.6 umol) and dioxane (3 mL) was sealed and heated to 90° C. for 2 h. Additional BrettPhos Pd G3 (15 mg, 16.6 umol) was added and the reaction was stirred at 90° C. for 2 h. The reaction was cooled to rt, diluted with EtOAc (5 mL), filtered through Celite®, rinsing with EtOAc (10 mL) and the filtrate concentrated to dryness. The crude material was purified via silica gel chromatography (heptane to 3:1 EtOAc:EtOH with 20 DMEA) to give tert-butyl methyl (5-(2-(dimethylamino)ethoxy)pyridine-2,4-diyl)dicarbamate as a light tan oil, (170 mg, 69.1% yield). LCMS m/z=355.0 [M+H]+. Preparations 244 to 246 The compounds in the following table were prepared from the appropriate 2-bromo or 2-chloropyridine and acetamide, following a similar procedure to that described in Preparation 41.

| Preparation | |
|---|---|
| No | Name, Structure, Starting Material (SM), Data |
| 244 | tert-butyl (2-acetamido-5-(oxetan-3-ylmethoxy)pyridin-4-yl)carbamate |
| | 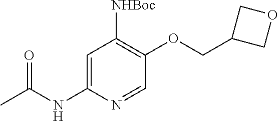 [SMILES: CC(=O)Nc1cc(NC(=O)OC(C)(C)C)c(OCC2COC2)cn1] |
| | SM: tert-butyl (2-bromo-5-(oxetan-3-ylmethoxy)pyridin-4-yl)carbamate |
| | (Preparation 228) |
| | 480 mg, 72.8% yield as a yellow solid. ¹H NMR: (500 MHz, CDCl₃) δ ppm: |
| | 8.78 (s, 1H), 7.06 (s, 1H), 4.90-4.95 (m, 2H), 4.54 (t, J = 6.0 Hz, 2H), 4.32 (d, |
| | J = 7.0 Hz, 2H), 3.42-3.50 (m, 1H), 2.17 (s, 3H), 1.55 (s, 9H). |
| 245 | tert-butyl (2-acetamido-5-(oxetan-2-ylmethoxy)pyridin-4-yl)carbamate |
| | 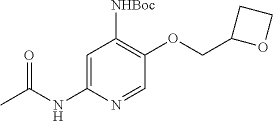 [SMILES: CC(=O)Nc1cc(NC(=O)OC(C)(C)C)c(OCC2CCO2)cn1] |
| | SM: tert-butyl (2-bromo-5-(oxetan-2-ylmethoxy)pyridin-4-yl)carbamate |
| | (Preparation 229) |
| | 448 mg, 36.7% yield as a white solid. ¹H NMR: (400 MHz, CDCl₃) δ ppm: |
| | 8.88 (br s, 1H), 8.07 (s, 1H), 7.84 (s, 1H), 7.69 (s, 1H), 5.14-5.17 (m, 1H), |
| | 4.69-4.78 (m, 1H), 4.65-4.67 (m, 1H), 4.22-4.25 (m, 1H), 4.06-4.09 (m, 1H), |
| | 2.78-2.83 (m, 1H), 2.67-2.70 (m, 1H), 2.16 (s, 3H), 1.53 (s, 9H). |
| 246 | tert-butyl (2-acetamido-5-(oxetan-3-yloxy)pyridin-4-yl)carbamate |
| | 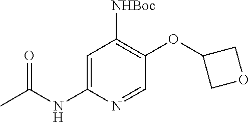 [SMILES: CC(=O)Nc1cc(NC(=O)OC(C)(C)C)c(OC2COC2)cn1] |
| | SM: tert-butyl (2-bromo-5-(oxetan-3-yloxy)pyridin-4-yl)carbamate |
| | (Preparation 230) |
| | 100 mg, 35.6% yield as a yellow solid. LCMS m/z = 324.1 [M + H]⁺ |

### Preparation 247

### tert-butyl (2-acetamido-5-hydroxypyridin-4-yl)carbamate

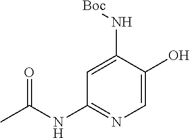 [SMILES: CC(=O)Nc1cc(NC(=O)OC(C)(C)C)c(O)cn1]

To a solution of tert-butyl (2-acetamido-5-(benzyloxy)pyridin-4-yl)carbamate (Preparation 38, 500 mg, 1.26 mmol) in MeOH (5 mL) was added Pd/C (250 mg, 10% purity) under N₂. The suspension was degassed under vacuum and purged with H₂ several times. The reaction mixture was stirred under H₂ (15 psi) at 20° C. for 12 h, then filtered and concentrated to give tert-butyl (2-acetamido-5-hydroxypyridin-4-yl)carbamate (480 mg) as a gray solid. LCMS m/z=212.3 [M+H]⁺.

### Preparation 248

### tert-butyl (2-acetamido-5-(2-(difluoromethoxy)ethoxy)pyridin-4-yl)carbamate

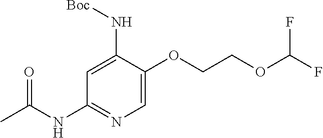 [SMILES: CC(=O)Nc1cc(NC(=O)OC(C)(C)C)c(OCCOC(F)F)cn1]

To a solution of tert-butyl (2-acetamido-5-hydroxypyridin-4-yl)carbamate (Preparation 247, 200 mg, 0.748 mmol) in DMF (10.0 mL) were added 2-(difluoromethoxy)ethyl 4-methylbenzenesulfonate (239 mg, 0.898 mmol) and Cs₂CO₃ (731 mg, 2.24 mmol). The reaction mixture was stirred at 25° C. for 2 h, then diluted with H₂O (50 mL), extracted with EtOAc (100 mL) and washed with brine (50 mL). The combined organic layers were concentrated under reduced pressure and purified by chromatography on silica gel (PE/EtOAc 3/1 to 0/1) to give tert-butyl (2-acetamido-5-(2-(difluoromethoxy)ethoxy)pyridin-4-yl)carbamate (170 mg, 62.9% yield) as a colourless oil. LCMS m/z=362.0 [M+H]⁺.

### Preparation 249

### tert-butyl (2-acetamido-5-(3-methoxypropoxy)pyridin-4-yl)carbamate

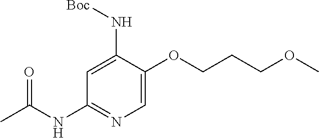 [SMILES: COCCCOc1cnc(NC(C)=O)cc1NC(=O)OC(C)(C)C]

To a solution of tert-butyl (2-acetamido-5-hydroxypyridin-4-yl)carbamate (Preparation 247, 330 mg, 1.11 mmol) in MeCN (6 mL) were added K₂CO₃ (461 mg, 3.33 mmol) and 3-methoxypropan-1-ol (680 mg, 4.44 mmol). The reaction mixture was stirred at 80° C. for 12 h, then diluted with H₂O (50 mL) and extracted with EtOAc (3×50 mL). The combined organic layers were dried over Na₂SO₄, filtered and concentrated under reduced pressure to give a residue which was purified by chromatography on silica gel (DCM/MeOH 100/1 to 25/2) to yield tert-butyl (2-acetamido-5-(3-methoxypropoxy)pyridin-4-yl)carbamate (400 mg, 95.5% yield) as red oil. LCMS m/z=340.2 [M+H]⁺.

### Preparation 250

### tert-butyl (2-acetamido-5-(2-morpholinoethoxy)pyridin-4-yl)carbamate

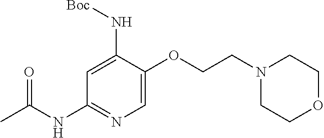 [SMILES: CC(=O)Nc1cc(NC(=O)OC(C)(C)C)c(OCCN2CCOCC2)cn1]

To a solution of tert-butyl (2-acetamido-5-hydroxypyridin-4-yl)carbamate (Preparation 247, 400 mg, 1.50 mmol) and K₂CO₃ (310 mg, 2.24 mmol) in MeCN (5.0 mL) was added 4-(2-chloroethyl)morpholine (HCl salt, 336 mg, 2.24 mmol). The reaction mixture was stirred at 80° C. for 1 h, then filtered and concentrated under reduced pressure. The residue was purified by chromatography on silica gel (DCM/MeOH 1/0 to 10/1) to give tert-butyl (2-acetamido-5-(2-morpholinoethoxy)pyridin-4-yl)carbamate (563 mg, 98.9% yield) as a yellow oil. LCMS m/z=381.2 [M+H]⁺.

### Preparation 251

### tert-butyl (2-acetamido-5-(2-(4-methylpiperazin-1-yl)ethoxy)pyridin-4-yl)carbamate

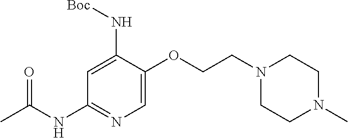 [SMILES: CC(=O)Nc1cc(NC(=O)OC(C)(C)C)c(OCCN2CCN(C)CC2)cn1]

A solution of tert-butyl (2-acetamido-5-hydroxypyridin-4-yl)carbamate (Preparation 247, 500 mg, 1.87 mmol), 2-(4-methylpiperazin-1-yl)ethan-1-ol (324 mg, 2.24 mmol) and (tributylphosphoranylidene)acetonitrile (2.3 g, 9.35 mmol) in toluene (5.0 mL) was stirred at 110° C. under N₂ for 1.5 h in a microwave. The mixture was hydrolysed with H₂O (30 mL) and extracted with EtOAc (3×30 mL). The organic layer was washed with brine (20 mL), dried over Na₂SO₄, and concentrated under reduced pressure to give a residue which was purified by chromatography on silica gel (PE/EtOAc 1/0 to 3/1) to yield tert-butyl (2-acetamido-5-(2-(4-methylpiperazin-1-yl)ethoxy)pyridin-4-yl)carbamate (200 mg, 27.20 yield) as a yellow oil. LCMS m/z=394.2 [M+H]⁺.

### Preparations 252 to 265

The compounds in the following table were prepared from the appropriate tert-butyl carbamate, following a similar procedure to that described in Preparation 63.

| Prepara- | |
|---|---|
| tion No | Name, Structure, Starting Material (SM), Data |
| 252 | N-(4-amino-5-((1-(trifluoromethyl)cyclopropyl)methoxy)pyridin-2- |
| | yl)acetamide |
| | 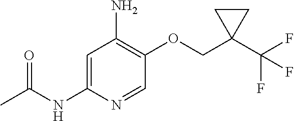 [SMILES: CC(=O)Nc1cc(N)c(OCC2(C(F)(F)F)CC2)cn1] |
| | SM: tert-butyl (2-acetamido-5-((1- |
| | (trifluoromethyl)cyclopropyl)methoxy)pyridin-4-yl)carbamate (Preparation |
| | 232) |
| | 148 mg, crude, HCl salt, as a white solid. LCMS m/z = 290.1 [M + H]⁺ |
| 253 | N-(4-amino-5-((2,2-difluorocyclopropyl)methoxy)pyridin-2-yl)acetamide |
| | 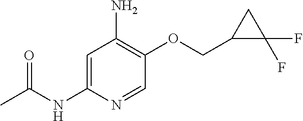 [SMILES: CC(=O)Nc1cc(N)c(OCC2CC2(F)F)cn1] |
| | SM: tert-butyl (2-acetamido-5-((2,2-difluorocyclopropyl)methoxy)pyridin-4- |
| | yl)carbamate (Preparation 233) |
| | 500 mg, crude, HCl salt, as a yellow solid. LCMS m/z = 258.1 [M + H]⁺ |
| 254 | N-(4-amino-5-((1-methylcyclopropyl)methoxy)pyridin-2-yl)acetamide |
| | 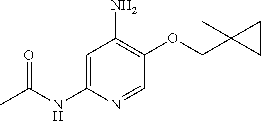 [SMILES: CC(=O)Nc1cc(N)c(OCC2(C)CC2)cn1] |
| | SM: tert-butyl (2-acetamido-5-((1-methylcyclopropyl)methoxy)pyridin-4- |
| | yl)carbamate (Preparation 234) |
| | 130 mg, crude, HCl salt, as a light yellow solid. LCMS m/z = 236.1 [M + H]⁺ |
| 254 | N-(4-amino-5-((1-cyanocyclopropyl)methoxy)pyridin-2-yl)acetamide |
| | 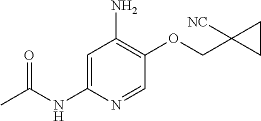 [SMILES: CC(=O)Nc1cc(N)c(OCC2(C#N)CC2)cn1] |
| | SM: tert-butyl (2-acetamido-5-((1-cyanocyclopropyl)methoxy)pyridin-4- |
| | yl)carbamate (Preparation 236) |
| | 140 mg, crude, HCl salt, as a yellow solid. LCMS m/z = 247.1 [M + H]⁺ |
| 255 | N-(4-amino-5-((1-methoxypropan-2-yl)oxy)pyridin-2-yl)acetamide |
| | 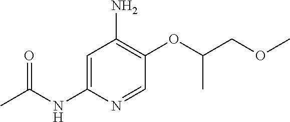 [SMILES: COCC(C)Oc1cnc(NC(C)=O)cc1N] |
| | SM: tert-butyl (2-acetamido-5-((1-methoxypropan-2-yl)oxy)pyridin-4- |
| | yl)carbamate (Preparation 237) |
| | 250 mg, crude, HCl salt, as a white solid. LCMS m/z = 240.2 [M + H]⁺ |
| 256 | N-(4-amino-5-(2-methoxypropoxy)pyridin-2-yl)acetamide |
| | 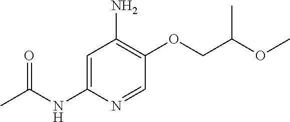 [SMILES: COC(C)COc1cnc(NC(C)=O)cc1N] |
| | SM: tert-butyl (2-acetamido-5-(2-methoxypropoxy)pyridin-4-yl)carbamate |
| | (Preparation 238) |
| | 120 mg, crude, HCl salt, as a white solid. LCMS m/z = 240.1 [M + H]⁺ |
| 257 | N-(4-amino-5-(2-(pyrrolidin-1-yl)ethoxy)pyridin-2-yl)acetamide |
| | 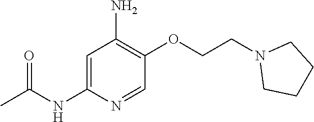 [SMILES: CC(=O)Nc1cc(N)c(OCCN2CCCC2)cn1] |
| | SM: tert-butyl (2-acetamido-5-(2-(pyrrolidin-1-yl)ethoxy)pyridin-4- |
| | yl)carbamate (Preparation 239) |
| | 140 mg, crude, HCl salt, as a white solid. |
| 258 | N-(4-amino-5-(cyclobutylmethoxy)pyridin-2-yl)acetamide |
| | 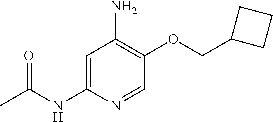 [SMILES: CC(=O)Nc1cc(N)c(OCC2CCC2)cn1] |
| | SM: tert-butyl (2-acetamido-5-(cyclobutylmethoxy)pyridin-4-yl)carbamate |
| | (Preparation 240) |
| | 176 mg, crude, HCl salt, as a white solid. LCMS m/z = 236.2 [M + H]⁺ |
| 259 | methyl (4-amino-5-(2-methoxyethoxy)pyridin-2-yl)carbamate |
| | 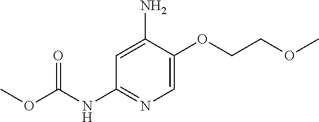 |
| | SM: tert-butyl methyl (5-(2-methoxyethoxy)pyridine-2,4-diyl)dicarbamate |
| | (Preparation 241) |
| | 300 mg, 84.9% yield, HCl salt, as a brown solid. ¹H NMR: (500 MHz, DMSO- |
| | d₆) δ ppm: 7.58 (s, 1H), 6.52 (s, 1H), 4.11-4.16 (m, 2H), 3.79 (s, 3H), 4.12-4.14 |
| | (m, 2H), 3.79 (s, 3H), 3.63-3.66 (m, 2H), 3.31 (s, 3H). |
| 260 | N-(4-amino-5-(2-methoxyethoxy)pyridin-2-yl)propionamide |
| | 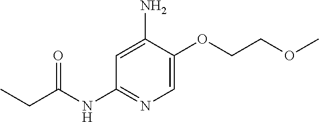 [SMILES: CCC(=O)Nc1cc(N)c(OCCOC)cn1] |
| | SM: tert-butyl (5-(2-methoxyethoxy)-2-propionamidopyridin-4-yl)carbamate |
| | (Preparation 242) |
| | 610 mg, 96.1% yield, HCl salt, as a brown solid. ¹H NMR: (500 MHz, DMSO- |
| | d₆) δ ppm: 7.57 (s, 1H), 6.66 (s, 1H), 4.11-4.14 (m, 2H), 3.67-3.72 (m, 2H), |
| | 3.28 (s, 3H), 1.06-1.12 (m, 3H), 0.90-0.96 (m, 2H). |
| 261 | N-(4-amino-5-(2-(difluoromethoxy)ethoxy)pyridin-2-yl)acetamide |
| | 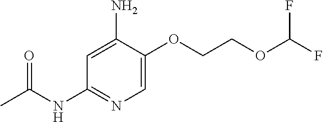 [SMILES: CC(=O)Nc1cc(N)c(OCCOC(F)F)cn1] |
| | SM: tert-butyl (2-acetamido-5-(2-(difluoromethoxy)ethoxy)pyridin-4- |
| | yl)carbamate |
| | (Preparation 248) |
| | 110 mg, crude, HCl salt, as a white solid. LCMS m/z = 262.0 [M + H]⁺ |
| 262 | N-(4-amino-5-(3-methoxypropoxy)pyridin-2-yl)acetamide |
| | 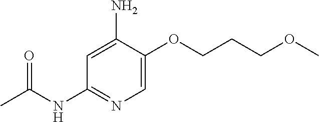 [SMILES: COCCCOc1cnc(NC(C)=O)cc1N] |
| | SM: tert-butyl (2-acetamido-5-(3-methoxypropoxy)pyridin-4-yl)carbamate |
| | (Preparation 249) |
| | 200 mg, 82.1% yield, HCl salt, as a light yellow solid. LCMS m/z = 240.2 |
| | [M + H]⁺ |
| 263 | N-(4-amino-5-(2-morpholinoethoxy)pyridin-2-yl)acetamide |
| | 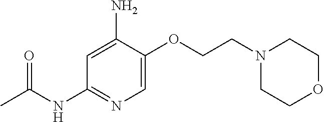 [SMILES: CC(=O)Nc1cc(N)c(OCCN2CCOCC2)cn1] |
| | SM: tert-butyl (2-acetamido-5-(2-morpholinoethoxy)pyridin-4-yl)carbamate |
| | Preparation 250) |
| | 130 mg, 88.2% yield, HCl salt, as a white solid. LCMS m/z = 281.1 [M + H]⁺ |
| 264 | N-(4-amino-5-(2-(4-methylpiperazin-1-yl)ethoxy)pyridin-2-yl)acetamide |
| | 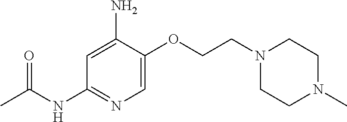 [SMILES: CC(=O)Nc1cc(N)c(OCCN2CCN(C)CC2)cn1] |
| | SM: tert-butyl (2-acetamido-5-(2-(4-methylpiperazin-1-yl)ethoxy)pyridin-4- |
| | yl)carbamate (Preparation 251) |
| | 150 mg, 100% yield, HCl salt, as a white solid. LCMS m/z = 294.1 [M + H]⁺ |
| 265 | methyl (4-amino-5-(2-(dimethylamino)ethoxy)pyridin-2-yl)carbamate |
| | hydrochloride |
| | 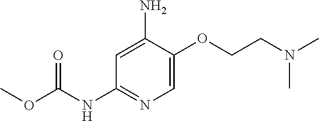 |
| | SM: tert-butyl methyl (5-(2-(dimethylamino)ethoxy)pyridine-2,4- |
| | diyl)dicarbamate (Preparation 243). |
| | LCMS m/z = 255.0 [M + H]⁺. |

### Preparation 266

### N-(4-amino-5-(oxetan-3-ylmethoxy)pyridin-2-yl)acetamide trifluoroacetate

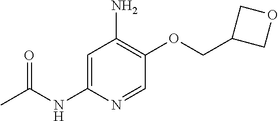 [SMILES: CC(=O)Nc1cc(N)c(OCC2COC2)cn1]

To a solution of tert-butyl (2-acetamido-5-(oxetan-3-ylmethoxy)pyridin-4-yl)carbamate (Preparation 244, 430 mg, 1.27 mmol) in HFIP (2.5 mL) was added TFA (205 mg, 1.80 mmol). The reaction mixture was stirred at 25° C. for 5 h, then concentrated under reduced pressure to obtain the N-(4-amino-5-(oxetan-3-ylmethoxy)pyridin-2-yl)acetamide, 740 mg, crude, TFA salt, as a white solid. LCMS m/z=238.0 [M+H]⁺.

### Preparations 267 to 268

The compounds in the following table were prepared as TFA salts from the appropriate tert-butyl carbamate, following a similar procedure to that described in Preparation 266.

| Preparation | |
|---|---|
| No | Name, Structure, Starting Material (SM), Data |
| 267 | N-(4-amino-5-(oxetan-2-ylmethoxy)pyridin-2-yl)acetamide |
| | 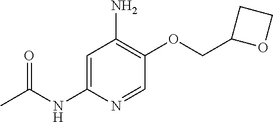 [SMILES: CC(=O)Nc1cc(N)c(OCC2CCO2)cn1] |
| | SM: tert-butyl (2-acetamido-5-(oxetan-2-ylmethoxy)pyridin-4-yl)carbamate |
| | (Preparation 245); DCM as solvent |
| | 486 mg, crude, as a yellow solid. LCMS m/z = 238.1 [M + H]⁺ |
| 268 | N-(4-amino-5-(oxetan-3-yloxy)pyridin-2-yl)acetamide |
| | 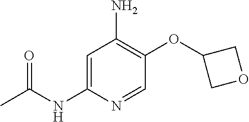 [SMILES: CC(=O)Nc1cc(N)c(OC2COC2)cn1] |
| | SM: tert-butyl (2-acetamido-5-(oxetan-3-yloxy)pyridin-4-yl)carbamate |
| | (Preparation 246) |
| | 100 mg, crude, as a light-yellow solid. LCMS m/z = 224.0 [M + H]⁺ |

### Preparation 269

### N-(4-amino-5-(1-cyclopropylethoxy)pyridin-2-yl)acetamide

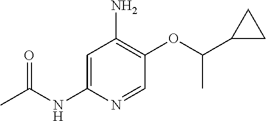 [SMILES: CC(=O)Nc1cc(N)c(OC(C)C2CC2)cn1]

To a solution of tert-butyl (2-acetamido-5-(1-cyclopropylethoxy)pyridin-4-yl)carbamate (Preparation 235, 135 mg, 0.40 mmol) in DCM (2 mL) were added 2,6-lutidine (86.3 mg, 0.80 mmol) and TMSOTf (160 mg, 0.60 mmol). The reaction mixture was stirred at 25° C. for 24 h under N₂, then concentrated and purified by prep-HPLC (Method B) to give compound
N-(4-amino-5-(1-cyclopropylethoxy)pyridin-2-yl)acetamide (84 mg, 0.36 mmol) as a white solid. LCMS m/z=236.1 [M+H]⁺.

### Preparation 270

### tert-butyl (2-acetamido-5-(2-fluoroethoxy)pyridin-4-yl)carbamate

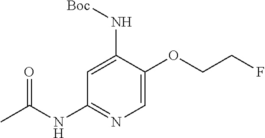 [SMILES: CC(=O)Nc1cc(NC(=O)OC(C)(C)C)c(OCCF)cn1]

To a solution of 2-fluoroethanol (110 μL, 1.87 mmol) in THF (10 mL) was added PPh₃ (736 mg, 2.81 mmol) followed by DIAD (553 μL, 2.81 mmol) and the solution stirred for 10 mins. tert-Butyl (2-acetamido-5-hydroxypyridin-4-yl)carbamate (Preparation 247, 0.500 g, 1.87 mmol) was added and the reaction was stirred at rt for 1 d. The reaction was diluted with water, extracted with EtOAc, the combined organic extracts washed with brine, dried over Na₂SO₄, filtered and evaporated. The residue was purified by silica gel chromatography (0-100% EtOAc in heptanes) to give tert-butyl (2-acetamido-5-(2-fluoroethoxy)pyridin-4-yl)carbamate (375 mg, 64% yield). LCMS m/z=314.1 [M+H]⁺.

### Preparation 271

### tert-butyl (2-acetamido-5-(3-fluoropropoxy)pyridin-4-yl)carbamate

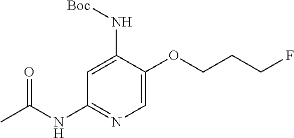 [SMILES: CC(=O)Nc1cc(NC(=O)OC(C)(C)C)c(OCCCF)cn1]

tert-Butyl (2-acetamido-5-(3-fluoropropoxy)pyridin-4-yl)carbamate was obtained, 438 mg, 71% from tert-butyl (2-acetamido-5-hydroxypyridin-4-yl)carbamate (Preparation 247) and 3-fluoropropan-1-ol, following the procedure described in Preparation 270. LCMS m/z=328.1 [M+H]⁺.

### Preparation 272

### N-(4-amino-5-(2-fluoroethoxy)pyridin-2-yl)acetamide hydrochloride

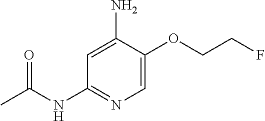

N-(4-amino-5-(2-fluoroethoxy)pyridin-2-yl)acetamide hydrochloride was obtained from tert-butyl (2-acetamido-5-(2-fluoroethoxy)pyridin-4-yl)carbamate (Preparation 270) following the procedure described in Preparation 63. LCMS m/z=214.1 [M+H]⁺.

### Preparation 273

### N-(4-amino-5-(3-fluoropropoxy)pyridin-2-yl)acetamide hydrochloride

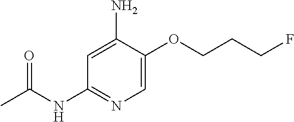 [SMILES: CC(=O)Nc1cc(N)c(OCCCF)cn1]

N-(4-amino-5-(3-fluoropropoxy)pyridin-2-yl)acetamide hydrochloride was obtained from tert-butyl (2-acetamido-5-(3-fluoropropoxy)pyridin-4-yl)carbamate (Preparation 271), following the procedure described in Preparation 63. LCMS m/z=228.1 [M+H]⁺.

### Preparation 274

### N-(2-chloro-5-(trifluoromethoxy)pyridin-4-yl)-6-cyclopropoxy-2-(1,1-difluoroethyl)pyrimidin-4-amine

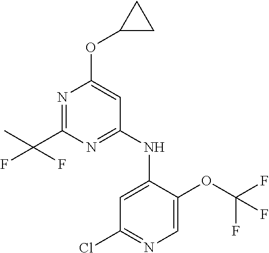 [SMILES: CC(F)(F)c1nc(Nc2cc(Cl)ncc2OC(F)(F)F)cc(OC2CC2)n1]

A vial containing 4-chloro-6-cyclopropoxy-2-(1,1-difluoroethyl)pyrimidine (Preparation 163, 325 mg, 1.4 mmol), 2-chloro-5-(trifluoromethoxy)pyridin-4-amine (196 mg, 0.923 mmol), Cs₂CO₃ (889 mg, 2.7 mmol), BINAP (138 mg, 0.222 mmol) and KOAc (24 mg, 0.106 mmol) in dioxane (4 mL) was degassed then backfilled with N₂ and heated to 95° C. After 1 h, the mixture was cooled to rt then loaded onto a silica gel column. The mixture was purified with (5-35% EtOAc in heptane) to afford N-(2-chloro-5-(trifluoromethoxy)pyridin-4-yl)-6-cyclopropoxy-2-(1,1-difluoroethyl)pyrimidin-4-amine as a white film (125 mg, 33% yield). LCMS m/z=410.8 [M+H]⁺.

### Preparation 275

### N-(4-((6-bromo-4-methylpyridin-2-yl)amino)-5-ethoxypyridin-2-yl)acetamide

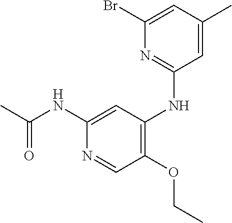 [SMILES: CCOc1cnc(NC(C)=O)cc1Nc1cc(C)cc(Br)n1]

To a mixture of N-(4-amino-5-ethoxypyridin-2-yl)acetamide hydrochloride (Preparation 62, 1 g, 4.32 mmol) in dioxane (25 mL) was added DIPEA (1.12 g, 8.63 mmol) and the reaction mixture was sonicated for 1 min. 2,6-Dibromo-4-methylpyridine (1.62 g, 6.47 mmol) was added, followed by Cs₂CO₃ (2.81 g, 8.63 mmol). A mixture of Pd(OAc)₂ (97 mg, 0.432 mmol) and BINAP (564 mg, 0.906 mmol) in dioxane (5 mL) was sonicated and then added to the reaction. The reaction mixture was purged with N₂, then heated at 90° C. for 3 h. The cooled reaction mixture was filtered through a Celite® plug, rinsing through with MeOH. The filtrate was concentrated and the crude was purified by chromatography on silica gel (50% EtOAc with 2% NH4OH in heptane) to give N-(4-((6-bromo-4-methylpyridin-2-yl)amino)-5-ethoxypyridin-2-yl)acetamide (512 mg, 32% yield). LCMS m/z=365, 367 [M+H]⁺

### Preparation 276

### tert-butyl (2-acetamido-5-ethoxypyridin-4-yl)carbamate

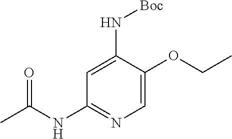 [SMILES: CCOc1cnc(NC(C)=O)cc1NC(=O)OC(C)(C)C]

To a solution of tert-butyl (2-bromo-5-ethoxypyridin-4-yl)carbamate (Preparation 23, 75 g, 236.5 mmol) in dioxane (1 L) was added acetamide (69.84 g, 1.18 mol), Cs₂CO₃ (231.13 g, 709.4 mmol) and BrettPhos Pd G3 (4.29 g, 4.73 mmol) and the reaction was stirred at 100° C. for 1 h under N₂. The cooled mixture was concentrated under reduced pressure. The residue was washed with water (500 mL) and extracted with EtOAC (500 mL×3). The combined organic layers were washed with brine (1000 mL), dried over Na₂SO₄, filtered and concentrated. The residue was purified on silica gel column chromatography (PE/EtOAc=5/1 to 1/1) to give tert-butyl (2-acetamido-5-ethoxypyridin-4-yl)carbamate (58 g, 83.1% yield) as yellow solid. 1H NMR (400 MHz, DMSO-d₆) δ ppm 10.17 (s, 1H), 8.62 (s, 1H), 8.09 (s, 1H), 7.91 (s, 1H), 4.11 (q, J=6.8 Hz, 2H), 2.03 (s, 3H), 1.48 (s, 9H), 1.35 (t, J=6.8 Hz, 3H).

### Preparation 277

### tert-butyl (2-acetamido-5-ethoxypyridin-4-yl)(6-chloro-2-(1,1-difluoroethyl)pyrimidin-4-yl)carbamate

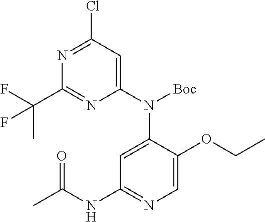 [SMILES: CCOc1cnc(NC(C)=O)cc1N(C(=O)OC(C)(C)C)c1cc(Cl)nc(C(C)(F)F)n1]

A mixture of tert-butyl (2-acetamido-5-ethoxypyridin-4-yl)carbamate (Preparation 276, 1 g, 3.39 mmol), 4,6-dichloro-2-(1,1-difluoroethyl)pyrimidine (Preparation 91), Cs₂CO₃ (4.5 g, 14 mmol), and DMF (10 mL) was heated to 40° C. for 6 h. The reaction was diluted with 1:1 heptane:EtOAc (50 mL) and washed with water (50 mL). The aqueous layer was extracted with 1:1 heptane:EtOAc (2×50 mL) and the combined organic layers were concentrated to dryness. The crude material was purified by silica gel chromatography (heptane to EtOAc) to give tert-butyl (2-acetamido-5-ethoxypyridin-4-yl)(6-chloro-2-(1,1-difluoroethyl)pyrimidin-4-yl)carbamate as a yellow solid. (880 mg, 55.1% yield). LCMS m/z=472.1 [M+H]⁺.

### Preparation 278

### tert-butyl (2-acetamido-5-methoxypyridin-4-yl)(6-chloro-2-(1,1-difluoroethyl)pyrimidin-4-yl)carbamate

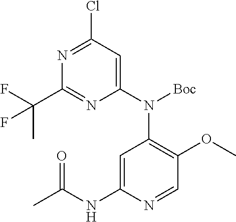 [SMILES: COc1cnc(NC(C)=O)cc1N(C(=O)OC(C)(C)C)c1cc(Cl)nc(C(C)(F)F)n1]

A solution of tert-butyl (2-acetamido-5-methoxypyridin-4-yl)carbamate (Preparation 35, 1 g, 3.55 mmol), Cs₂CO₃ (3.5 g, 10.74 mmol) and DMAC (20 mL) was stirred for 10 mins at rt then 4,6-dichloro-2-(1,1-difluoroethyl)pyrimidine (Preparation 91, 1.5 g, 7.04 mmol) was added and the reaction stirred at rt for 4 h. The reaction was diluted with EtOAc (50 mL) and filtered through Celite®. The filtrate was concentrated to a low volume, then dissolved in 1:1 heptane:EtOAc (100 mL) and washed with 10% brine/water (2×150 mL). The crude organic layer was concentrated to dryness and purified by silica gel chromatography (heptane to EtOAc) to give tert-butyl (2-acetamido-5-methoxypyridin-4-yl)(6-chloro-2-(1,1-difluoroethyl)pyrimidin-4-yl)carbamate as a bright yellow oil (1 g, 61.4% yield).

### Preparation 279

### N-(4-((2-(1,1-difluoroethyl)-6-methylpyrimidin-4-yl)amino)-5-hydroxypyridin-2-yl)acetamide

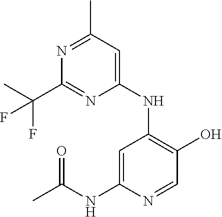 [SMILES: CC(=O)Nc1cc(Nc2cc(C)nc(C(C)(F)F)n2)c(O)cn1]

To a solution of N-(5-(benzyloxy)-4-((2-(1,1-difluoroethyl)-6-methylpyrimidin-4-yl)amino)pyridin-2-yl)acetamide (Example 30, 12.6 g, 30.5 mmol) in EtOH (150 mL) was added Pd/C (324 mg, 3.05 mmol). The mixture was stirred under H₂ atmosphere (15 psi) at 25° C. for 16 h, then filtered and concentrated to give a residue which was purified by column chromatography on silica gel (DCM/MeOH 1/0 to 10/1) to give N-(4-((2-(1,1-difluoroethyl)-6-methylpyrimidin-4-yl)amino)-5-hydroxypyridin-2-yl)acetamide (10 g, 79.2% yield) as white solid. LCMS m/z=324.1 [M+H]⁺.

### Preparation 280

### N-(5-(benzyloxy)-4-((6-cyclopropoxy-2-(1,1-difluoroethyl)pyrimidin-4-yl)amino)pyridin-2-yl)acetamide

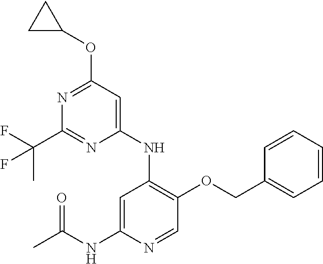 [SMILES: CC(=O)Nc1cc(Nc2cc(OC3CC3)nc(C(C)(F)F)n2)c(OCc2ccccc2)cn1]

To a solution of N-(4-amino-5-(benzyloxy)pyridin-2-yl)acetamide hydrochloride (Preparation 82, 1.38 g, 4.69 mmol) and 4-chloro-6-cyclopropoxy-2-(1,1-difluoroethyl)pyrimidine (Preparation 163, 1 g, 4.26 mmol) in dioxane (60 mL) were added Cs₂CO₃ (2.78 g, 8.52 mmol), BINAP (530.8 mg, 0.852 mmol) and Pd₂(dba)₃ (390.3 mg, 0.426 mmol) and the reaction degassed for 10 mins by bubbling N₂. The reaction was stirred at 90° C. for 8 h. The cooled reaction was diluted with water (50 mL) and extracted with EtOAc (50 mL×3). The combined organic extracts was dried over Na₂SO₄, filtered and concentrated in vacuo. The crude material was purified by silica gel chromatography (PE/EtOAc=5/1-1/1) to give N-(5-(benzyloxy)-4-((6-cyclopropoxy-2-(1,1-difluoroethyl)pyrimidin-4-yl)amino)pyridin-2-yl)acetamide (1.05 g, 54.1% yield) as a white solid. LCMS m/z=456.2 [M+H]+

### Preparation 281

### N-(4-((6-cyclopropoxy-2-(1,1-difluoroethyl)pyrimidin-4-yl)amino)-5-hydroxypyridin-2-yl)acetamide

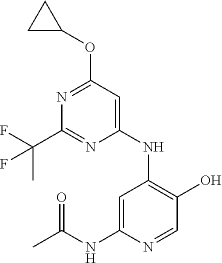 [SMILES: CC(=O)Nc1cc(Nc2cc(OC3CC3)nc(C(C)(F)F)n2)c(O)cn1]

To a solution of N-(5-(benzyloxy)-4-((6-cyclopropoxy-2-(1,1-difluoroethyl)pyrimidin-4-yl)amino)pyridin-2-yl)acetamide (Preparation 280, 15 g, 32.9 mmol) in EtOH (180 mL) was added Pd/C (3.50 g, 3.29 mmol, 10% purity). The reaction mixture was stirred at 30° C., then purged by bubbling H₂ for 10 mins. The reaction was warmed to 50° C. and stirred under H₂ of 15 psi for 4 h. The reaction was purged by bubbling N₂ for 10 mins, then filtered through Celite®, rinsing with EtOH (80 mL). The filtrate was evaporated under reduced pressure to give filtrate to give N-(4-((6-cyclopropoxy-2-(1,1-difluoroethyl)pyrimidin-4-yl)amino)-5-hydroxypyridin-2-yl)acetamide (10.2 g, 84.8% yield) a tan solid. LCMS m/z=366.0 [M+H]+; ¹H NMR (400 MHz, DMSO-d₆) δ 10.08 (s, 2H), 9.30 (s, 1H), 8.90 (br s, 1H), 7.82 (s, 1H), 6.91 (s, 1H), 4.10-4.19 (m, 1H), 1.98-2.13 (m, 6H), 0.68-0.95 (m, 4H).

### Preparation 282

### 4-iodo-2-(2-methoxyethoxy)pyrimidine

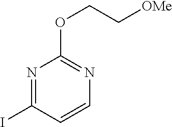 [SMILES: COCCOc1nccc(I)n1]

To a solution of 2-chloro-4-iodopyrimidine (142 mg, 1.87 mmol) in THF (5 mL) was added NaH (99.8 mg, 2.50 mmol, 60% purity) at 25° C. and the mixture stirred for 30 min before 2-methoxyethan-1-ol (300 mg, 1.25 mmol) was added and the mixture was stirred at 70° C. for 16 h. The mixture was concentrated and the residue purified by chromatography on silica gel (33% EtOAc/PE) to give 4-iodo-2-(2-methoxyethoxy)pyrimidine as a yellow oil (150 mg, 43%). ¹H NMR (400 MHz, CDCl₃) δ ppm 8.01 (d, J=5.2 Hz, 1H), 7.39 (d, J=5.2 Hz, 1H), 4.50-4.52 (m, 2H), 3.75-3.77 (m, 2H), 3.42 (s, 3H).

### Preparation 283

### 2-chloro-4-(2-methoxyethoxy)pyrimidine

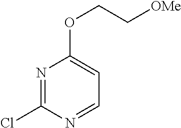 [SMILES: COCCOc1ccnc(Cl)n1]

To a solution of 2,4-dichloropyrimidine (1 g, 6.71 mmol) in DMF (15 mL) was added 2-methoxyethan-1-ol (511 mg, 6.71 mmol) and Cs₂CO₃ (4.4 g, 13.42 mmol) and the mixture stirred at 100° C. for 4 h. The mixture was quenched with H₂O (20 mL) and extracted with EtOAc (20 mL×2). The combined organics were washed with brine (20 mL×2), dried (Na₂SO₄) and were concentrated under reduced pressure. The residue was purified by chromatography on silica gel (EtOAc) to give 2-chloro-4-(2-methoxyethoxy)pyrimidine as a yellow solid (700 mg, 55%). ¹H NMR (400 MHz, CDCl₃) δ ppm 8.30 (d, J=6.0 Hz, 1H), 6.73 (d, J=5.6 Hz, 1H), 4.52-4.56 (m, 2H), 3.71-3.79 (m, 2H), 3.43 (s, 3H).

### Preparation 284

### 6-(2-methoxyethyl)pyrimidin-4-ol

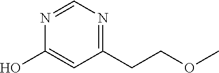

To a solution of methyl 5-methoxy-3-oxopentanoate (1 g, 6.24 mmol) in MeOH (10 mL) was added formimidamide acetate (650 mg, 6.24 mmol) and NaOMe (674.6 mg, 12.49 mmol) and the mixture was stirred at 70° C. for 2 h. The mixture was concentrated and purified by Prep-HPLC (Method E, Gradient 0-30%) to give 6-(2-methoxyethyl)pyrimidin-4-ol (135 mg, 14.0% yield) as a yellow oil. ¹H NMR (400 MHz, CDCl₃) δ ppm 8.38 (s, 1H), 6.48 (s, 1H), 5.87-5.98 (m, 1H), 3.73 (t, J=5.8 Hz, 2H), 3.35 (s, 3H), 2.88 (t, J=5.8 Hz, 2H).

### Preparation 285

### 4-chloro-6-(2-methoxyethyl)pyrimidine

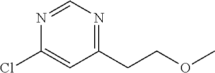

To a solution of 6-(2-methoxyethyl)pyrimidin-4-ol (Preparation 284, 100 mg, 0.649 mmol) in DCM (3 mL) was added POCl₃ (1.0 mL 10.73 mmol) and the mixture was stirred at 40° C. for 18 h. The mixture was evaporated, the residue diluted with water (10 mL) and extracted with EtOAc (20 mL×3). The combined organic layers were washed with brine (25 mL), dried over Na₂SO₄, filtered and concentrated to give a residue which was purified on silica gel column chromatography (PE/EtOAc=1/0 to 1/1) to give 4-chloro-6-(2-methoxyethyl)pyrimidine (40 mg, 35.7% yield) as a yellow oil. ¹H NMR: (400 MHz, CDCl₃) δ ppm 9.08 (s, 1H), 7.57 (s, 1H), 3.77-3.81 (m, 2H), 3.35 (s, 3H), 3.18-3.22 (m, 2H).

### Preparation 286

### 2-chloro-5-(difluoromethoxymethyl)pyrazine

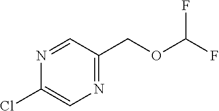 [SMILES: FC(F)OCc1cnc(Cl)cn1]

(5-Chloropyrazin-2-yl)methanol (500 mg, 3.46 mmol) and CuI (329.36 mg, 1.73 mmol) were dissolved in MeCN (5 mL) and the reaction mixture was heated to 45° C. A solution of 2,2-difluoro-2-fluorosulfonyl-acetic acid (924 mg, 5.19 mmol) in MeCN (3 mL) was added dropwise over 30 mins. The reaction was stirred for 3 h, cooled to rt and diluted with EtOAc. The organic layer was washed with sat. NaHCO₃ solution and dried over Na₂SO₄. The crude product was purified by silica gel column chromatography eluting with (0-100% heptane-EtOAc) to obtain 2-chloro-5-(difluoromethoxymethyl)pyrazine (120 mg, 14.3% yield).

### Preparation 287

### 1-((2,6-dibromopyridin-3-yl)oxy)-2-methylpropan-2-ol

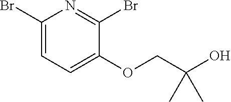

To a solution of 2,6-dibromopyridin-3-ol (10 g, 39.54 mmol) in DMF (150 mL) was added K₂CO₃ (16.40 g, 118.6 mmol) and 2,2-dimethyloxirane (5.70 g, 79.1 mmol). The mixture was stirred at 100° C. for 24 h under N₂. The mixture was diluted with water (100 mL), extracted with EtOAc (100 mL×3), dried over Na₂SO₄, filtered and concentrated under reduced pressure. The residue was purified by silica gel chromatography (PE/EtOAc=100/1 to 3/1) to give 1-((2,6-dibromopyridin-3-yl)oxy)-2-methylpropan-2-ol (8 g, 56.0% yield) as a brown oil. 1H NMR (400 MHz, CDCl₃) δ ppm 7.37 (d, J=8.4 Hz, 1H), 7.03 (d, J=8.4 Hz, 1H), 3.84 (s, 2H), 1.39 (s, 6H).

### Preparation 288

### 6-bromo-2,2-dimethyl-2,3-dihydro-[1,4]dioxino[2,3-b]pyridine

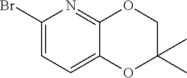

To a solution of 1-((2,6-dibromopyridin-3-yl)oxy)-2-methylpropan-2-ol (Preparation 287, 8 g, 22.15 mmol) in DMF (120 mL) was added NaH (1.33 g, 33.23 mmol, 60% purity) at 0° C. The mixture was stirred at 90° C. for 3 h under N₂. The reaction mixture was quenched with H₂O (20 mL) and extracted with EtOAc (100 mL×3). The combined organic layers were washed with brine (100 mL), dried over Na₂SO₄, filtered and concentrated under reduced pressure. The residue was purified by silica gel chromatography (PE/EtOAc=100/1 to 5/1) to give 6-bromo-2,2-dimethyl-2,3-dihydro-[1,4]dioxino[2,3-b]pyridine (4.2 g, 69.9% yield) as white solid. 1H NMR: (400 MHz, CDCl₃) δ ppm 6.99-7.03 (m, 2H), 4.06 (s, 2H), 1.36 (s, 6H).

### Preparation 289

### 3-bromo-1-(2-ethoxyethyl)-1H-pyrazole

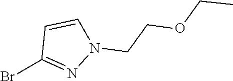

To a solution of 3-bromo-1H-pyrazole (300 mg, 1.96 mmol) in MeCN (10 mL) was added 1-bromo-2-ethoxyethane (288.2 mg, 1.96 mmol), Cs₂CO₃ (638.8 mg, 1.96 mmol) and KI (65.1 mg, 0.39 mmol) and the resulting mixture was stirred at 90° C. for 8 h. The mixture was concentrated under vacuum and the crude was purified by column chromatography (PE/EtOAc=0/1 to 3/1) to give 3-bromo-1-(2-ethoxyethyl)-1H-pyrazole (230 mg, 53.6% yield) as white oil. ¹H NMR (400 MHz, CDCl₃) δ ppm 7.38 (d, J=2.4 Hz, 1H), 6.24 (d, J=2.0 Hz, 1H), 4.24 (t, J=5.2 Hz, 2H), 3.74 (t, J=5.2 Hz, 2H), 3.41-3.47 (m, 2H), 1.15 (t, J=6.8 Hz, 3H).

### Preparation 290

### 4-bromo-2-(2-methoxyethyl)-2H-1,2,3-triazole and 4-bromo-1-(2-methoxyethyl)-1H-1,2,3-triazole

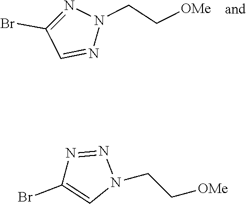 [SMILES: COCCn1ncc(Br)n1 ; COCCn1cc(Br)nn1]

To a solution of 4-bromo-2H-1,2,3-triazole (1.0 g 6.67 mmol) in DMF (10 mL) was added 1-bromo-2-methoxyethane (1.03 g, 7.43 mmol) and Cs₂CO₃ (4.4 g, 13.5 mmol) and the resulting mixture stirred at 80° C. for 2 h. The reaction mixture was diluted with water (50 mL) and extracted with DCM (30 mL×3). The combined organics were washed with brine (30 mL×2), dried (Na₂SO₄) and evaporated to dryness to afford a mixture of 4-bromo-2-(2-methoxyethyl)-2H-1,2,3-triazole and 4-bromo-1-(2-methoxyethyl)-1H-1,2,3-triazole as a yellow oil (1.2 g, 86%). ¹H NMR (400 MHz, CDCl₃) δ ppm: 7.70 (s, 0.3H), 7.56 (s, 1H), 4.57 (t, J=5.6 Hz, 2H), 4.53 (t, J=4.8 Hz, 0.7H), 3.87 (t, J=5.6 Hz, 2H), 3.74 (t, J=4.8 Hz, 0.7H), 3.36 (s, 1H), 3.35 (s, 3H).

### Preparation 291

### 3-bromo-1-(2-methoxyethyl)-1H-1,2,4-triazole

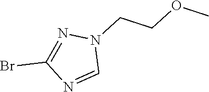

3-Bromo-1-(2-methoxyethyl)-1H-1,2,4-triazole was obtained as a yellow solid, 500 mg, 71.8%, from 3-bromo-1H-1,2,4-triazole and 1-bromo-2-methoxyethane (469.7 mg, 3.38 mmol) following the procedure described in Preparation 290. ¹H NMR (400 MHz, CDCl₃) δ: ppm 8.02 (s, 1H), 4.28-4.30 (m, 2H), 3.69-3.74 (m, 2H), 3.33 (s, 3H).

### Preparation 292

### 2-(3-bromo-1-methyl-1H-pyrazol-5-yl)propan-2-ol

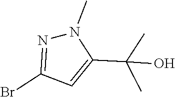

To a solution of 1-(3-bromo-1-methyl-1H-pyrazol-5-yl)ethan-1-one (1 g, 4.93 mmol) in THF (10 mL) was added MeMgBr (3 M, 2.46 mL, 7.39 mmol) at 0° C. under N₂. The mixture was stirred at 20° C. under N₂ for 2 h. The mixture was quenched with water (10 mL) and extracted with EtOAc (20 mL×3). The combined organic layers were washed with brine (40 mL), dried over Na₂SO₄, filtered and concentrated to give a residue. The residue was purified by silica gel chromatography (PE/EtOAc=100/1 to 10/7) to give 2-(3-bromo-1-methyl-1H-pyrazol-5-yl)propan-2-ol (1 g, 92.7% yield) as a light yellow liquid. LCMS m/z=219.1 [M+H]⁺

### Preparation 293

### 2-bromo-5-(methoxymethyl)thiazole

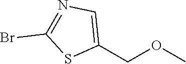

To a solution of 2-bromo-5-(hydroxymethyl)thiazole (1.0 g, 5.15 mmol) in THF (20 mL) was added NaH (309.2 mg, 7.73 mmol, 60% purity) at 0° C. and the mixture stirred at 25° C. for 1 h. MeI (877.8 mg, 6.18 mmol) was added and the reaction stirred at 25° C. for 16 h. The mixture was diluted with water (20 mL) and extracted with EtOAc (30 mL×3). The combined organic phase was washed with brine (30 mL×3), dried over anhydrous Na₂SO₄, filtered and concentrated. The crude was purified by column chromatography (PE/EtOAc=1/0 to 5/1) to give 2-bromo-5-(methoxymethyl)thiazole (200.0 mg, 18.65% yield) as light-yellow oil. LCMS m/z=207.9 [M+H]⁺

### Preparation 294 to 300

The compounds in the following table were prepared from the appropriate alcohol and MeI, following a similar procedure to that described in Preparation 293.

| Preparation | |
|---|---|
| No | Name, Structure, Starting Material (SM), Data |
| 294 | 5-bromo-2-(methoxymethyl)thiazole |
| | 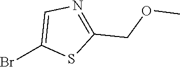 |
| | SM: 5-bromo-2-(hydroxymethyl)thiazole |
| | yellow oil, 236 mg, 73.2%. 1H NMR (400 MHz, CDCl₃) δ ppm: 7.29 (s, 1H), |
| | 4.75 (s, 2H), 3.53 (s, 3H). |
| 295 | 2-bromo-4-(methoxymethyl)thiazole |
| | 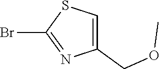 |
| | SM: 2-bromo-4-(hydroxymethyl)thiazole |
| | yellow oil, 149 mg, 46.3%. 1H NMR (400 MHz, CDCl₃) δ ppm 7.18 (s, 1H), |
| | 4.54 (s, 2H), 3.46 (s, 3H). |
| 296 | 2-bromo-5-(2-methoxyethyl)thiazole |
| | 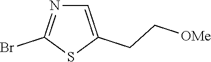 |
| | SM: 2-(2-bromothiazol-5-yl)ethan-1-ol (40 mg, 25%) as a yellow oil. ¹H |
| | NMR (400 MHz, DMSO-d₆) δ ppm: 7.47 (d, J = 4.0 Hz, 1H), 3.49-3.53 (m, |
| | 2H), 3.28 (s, 3H), 3.01-3.05 (m, 2H). |
| 297 | 3-iodo-1-(2-methoxy-2-methylpropyl)-1H-pyrazole |
| | 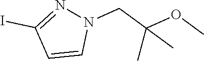 |
| | SM: 1-(3-iodo-1H-pyrazol-1-yl)-2-methylpropan-2-ol (Example 82, step 2, |
| | US11590111B2) |
| | (280.6 mg, 84.4% yield) as yellow oil. ¹H NMR (400 MHz, CDCl₃) δ ppm: |
| | 7.32 (d, J = 2.4 Hz, 1H), 6.40 (d, J = 2.0 Hz, 1H), 4.12 (s, 2H), 3.22 (s, 3H), |
| | 1.12 (s, 6H). |
| 298 | 3-bromo-5-(2-methoxypropan-2-yl)-1-methyl-1H-pyrazole |
| | 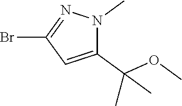 |
| | SM: 2-(3-bromo-1-methyl-1H-pyrazol-5-yl)propan-2-ol (Preparation 292) |
| | pale yellow oil (150 mg, 50%). ¹H NMR (400 MHz, CDCl₃) δ ppm: 6.10 (s, |
| | 1H), 3.97 (s, 3H), 3.08 (s, 3H), 1.54 (s, 6H). |
| 299 | 3-bromo-5-(methoxymethyl)-1-methyl-1H-pyrazole |
| | 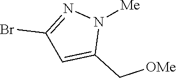 |
| | SM: (3-bromo-1-methyl-1H-pyrazol-5-yl)methanol |
| | white solid (345 mg, 92%). ¹H NMR (400 MHz, DMSO-d₆) δ ppm: 6.37 (s, |
| | 1H), 4.03 (s, 2H), 3.36 (s, 3H), 1.63 (s, 3H). |
| 300 | 2-chloro-6-(2-methoxyethyl)pyrazine |
| | 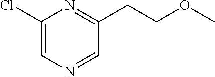 |
| | SM: 2-(6-chloropyrazin-2-yl)ethanol |
| | (100 mg, 30.6% yield). LCMS m/z = 173.0 [M + H]⁺ |

### Preparation 301

### 5-bromo-N,N-dimethyl-1,3,4-thiadiazol-2-amine

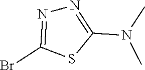

To a solution of 2,5-dibromo-1,3,4-thiadiazole (500 mg, 2.05 mmol) and N-methylmethanamine hydrochloride (200.6 mg, 2.46 mmol) in dioxane (10 mL) was added TEA (414.9 mg, 4.10 mmol). The mixture was stirred at 100° C. for 2 h. The mixture was concentrated and purified by chromatography on silica gel (PE/EtOAc=100/1 to 3/1) to give 5-bromo-N,N-dimethyl-1,3,4-thiadiazol-2-amine (360 mg, 76.0% yield) as a white solid. LCMS m/z=208.0 [M+H]⁺

### Preparation 302

### 4-bromo-N N-dimethylthiazol-2-amine

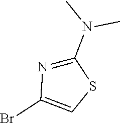

To a solution of 2,4-dibromothiazole (500 mg, 2.06 mmol) in MeCN (1 mL) was added dimethylamine (928.0 mg, 20.58 mmol) and DBU (940.1 mg, 6.17 mmol) and the reaction mixture was stirred at 20° C. for 16 h. The mixture was concentrated under vacuum and the crude was purified by column chromatography (PE/EtOAc=0/1 to 1/1) to give 4-bromo-N,N-dimethylthiazol-2-amine (300 mg, 70.4% yield) as a white solid. ¹H NMR (400 MHz, CDCl₃) δ ppm 6.35 (s, 1H), 3.10 (s, 6H).

### Preparation 303

### 2-(3-bromo-1-methyl-1H-pyrazol-5-yl)-N,N-dimethylpropan-2-amine

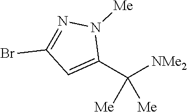 [SMILES: CN(C)C(C)(C)c1cc(Br)nn1C]

Part A: To a solution of 2-(3-bromo-1-methyl-1H-pyrazol-5-yl)propan-2-ol (Preparation 292, 650 mg, 2.97 mmol) in MeCN (6 mL) was added sulfuric acid (1.5 mL) at 0° C., and the mixture stirred at 25° C. for 15 h. After work-up, N-(2-(3-bromo-1-methyl-1H-pyrazol-5-yl)propan-2-yl)acetamide was obtained as a yellow solid (610 mg, 71%)

Part B: A solution of N-(2-(3-bromo-1-methyl-1H-pyrazol-5-yl)propan-2-yl)acetamide (500 mg, 1.92 mmol) in HCl (4.8 mL), H₂O (10 mL) and MeOH (10 mL) was stirred at 80° C. for 12 h. The mixture was concentrated and purified by prep-HPLC (Method X, Gradient 0-25%) to give 2-(3-bromo-1-methyl-1H-pyrazol-5-yl)propan-2-amine as a yellow oil (205 mg, 44%). LCMS m/z=220.1 [M+H]⁺.

Part C: A solution of 2-(3-bromo-1-methyl-1H-pyrazol-5-yl)propan-2-amine (170 mg, 0.78 mmol) and (CH₂O)n (3.74 g, 3.12 mmol, 4.25 mL) in MeOH (2 mL) and H₂O (0.2 mL) was stirred at 25° C. for 1 h before NaBH₃CN (196 mg, 3.12 mmol) and ZnCl₂ (106 mg, 0.78 mmol) was added and the reaction stirred at 25° C. for 12 h. The reaction mixture was evaporated to dryness and purified by chromatography on silica gel (1-25% EtOAc/PE) to give 2-(3-bromo-1-methyl-1H-pyrazol-5-yl)-N,N-dimethylpropan-2-amine as a yellow oil (110 mg, 57%)¹H NMR (500 MHz, DMSO-d₆) δ ppm 6.19 (s, 1H), 3.96 (s, 3H), 2.08 (s, 6H), 1.30 (s, 6H).

### Preparation 304

### 3-bromo-1-methyl-5-(pyrrolidin-1-ylmethyl)-1H-pyrazole

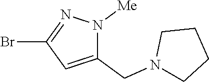 [SMILES: Cn1nc(Br)cc1CN1CCCC1]

To a solution of 3-bromo-1-methyl-1H-pyrazole-5-carbaldehyde (300 mg, 1.59 mmol) in MeOH (6 mL) was added pyrrolidine (339 mg, 4.76 mmol) and NaBH₃CN (499 mg, 7.94 mmol) and the resulting mixture was stirred at 70° C. for 16 h. The mixture was concentrated and the residue purified by prep-HPLC (Method R, Gradient 26-56%) to give 3-bromo-1-methyl-5-(pyrrolidin-1-ylmethyl)-1H-pyrazole as a yellow oil (266 mg, 69%). ¹H NMR (500 MHz, CDCl₃) δ ppm: 6.13 (s, 1H), 3.85 (s, 3H), 3.57 (s, 2H), 2.47-2.49 (s, 4H), 1.76-1.80 (s, 4H).

### Preparation 305 to 308

The title compounds were prepared from the appropriate aldehyde and amine using an analogous method to that described for Preparation 304.

| Preparation | Name/Structure/SM/Data |
|---|---|
| 305 | 1-((3-bromo-1-methyl-1H-pyrazol-5-yl)methyl)-4-methylpiperazine |
| | 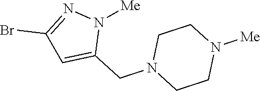 [SMILES: CN1CCN(Cc2cc(Br)nn2C)CC1] |
| | SM: 3-bromo-1-methyl-1H-pyrazole-5-carbaldehyde |
| | ¹H NMR (400 MHz, CDCl₃) δ ppm: 6.14 (s, 1H), 3.85 (s, 3H), 3.45 (s, 2H), |
| | 2.30-2.56 (m, 8H), 2.28 (s, 3H). |
| 306 | 2-bromo-4-(pyrrolidin-1-ylmethyl)pyridine |
| | 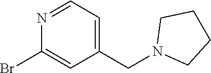 [SMILES: Brc1cc(CN2CCCC2)ccn1] |
| | SM: 2-bromoisonicotinaldehyde |
| | ¹H NMR (400 MHz, CDCl₃) δ ppm: 8.29 (d, J = 5.2 Hz, 1H), 7.50 (s, 1H), |
| | 7.25 (d, J = 5.2 Hz, 1H), 3.60 (s, 2H), 2.53 (s, 4H), 1.82 (s, 4H). |
| 307 | 4-((2-bromopyridin-4-yl)methyl)morpholine |
| | 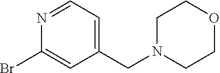 [SMILES: Brc1cc(CN2CCOCC2)ccn1] |
| | SM: 2-bromoisonicotinaldehyde |
| | LCMS m/z = 257.1 [M + H]⁺. |
| 308 | 1-(5-chloropyrazin-2-yl)-N,N-dimethylmethanamine |
| | 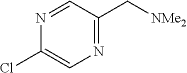 [SMILES: CN(C)Cc1cnc(Cl)cn1] |
| | SM: 5-chloropyrazine-2-carbaldehyde |
| | LCMS m/z = 172.2 [M + H]⁺. |

### Preparation 309

### 2-bromo-5-(2-methoxyethyl)-1,3,4-thiadiazole

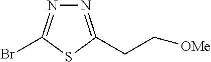

Part A: A solution of 3-methoxypropanenitrile (1 g, 11.7 mmol) and hydrazinecarbothioamide (1.29 g, 14.1 mmol) in TFA (10 mL) was stirred at 65° C. for 5 h. The mixture was concentrated and purified by chromatography on silica gel (150 EtOAc/PE) to give 5-(2-methoxyethyl)-1,3,4-thiadiazol-2-amine as a yellow oil (580 mg, 28%).

Part B: Isopentyl nitrite (883 mg, 7.54 mmol) and TMSBr (865 mg, 5.65 mmol) were added to a solution of 5-(2-methoxyethyl)-1,3,4-thiadiazol-2-amine (300 mg, 1.88 mmol) in CH₂Br₂ (2 mL) at 0° C. and the reaction stirred for 30 min at 0° C. and at 25° C. for 2 h. The mixture was concentrated and purified by chromatography on silica gel (1-25% EtOAc/PE) to give 2-bromo-5-(2-methoxyethyl)-1,3,4-thiadiazole as a yellow oil (290 mg, 62%). LCMS m/z=222.9 [M+H]⁺.

### Preparation 310

### 2-bromo-5-(methoxymethyl)-1,3,4-thiadiazole

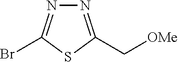

The title compound was prepared from 5-(methoxymethyl)-1,3,4-thiadiazol-2-amine using an analogous method used to prepare Preparation 309, Part B. LCMS m/z=208.9 [M+H]⁺.

### Preparation 311

### 3-bromo-N,N-dimethyl-1,2,4-thiadiazol-5-amine

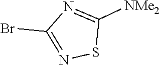

Dimethylamine (186.5 mg, 1.65 mmol) and DIPEA (583 mg, 4.51 mmol) were added to a solution of 3-bromo-5-chloro-1,2,4-thiadiazole (300 mg, 1.50 mmol) in EtOH (10 mL) and the mixture stirred at 25° C. for 1 h. The mixture was concentrated and the residue purified by silica column (0-20% EtOAc/PE) to give 3-bromo-N,N-dimethyl-1,2,4-thiadiazol-5-amine as a colourless liquid (262 mg, 84%). LCMS m/z=210.0 [M+H]⁺.

### Preparation 312

### 5-bromo-3-(methoxymethyl)-1,2,4-thiadiazole

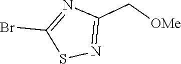

Part A: To a solution of 2-methoxyacetimidam ide hydrochloride (500 mg, 4.01 mmol) in MeOH (15 mL) at 0° C., under vigorous stirring, was added dropwise Br₂ (641.4 mg, 4.01 mmol) and NaOMe (217 mg, 4.01 mmol) simultaneously over 30 min maintaining a slight bromine excess by color. To the resulting nearly colorless suspension was added dropwise a solution of potassium thiocyanate (390 mg, 4.01 mmol) in MeOH (5 mL) over 10 min at 0-10° C. The resulting mixture was stirred for 2 h at 0-10° C. The solid was collected by filtration and washed with MeOH to afford a brown solid which was purified by chromatography on silica gel (DCM/MeOH=10/1) to give 3-(methoxymethyl)-1,2,4-thiadiazol-5-amine (280 mg, 48%).

Part B: To a solution of 3-(methoxymethyl)-1,2,4-thiadiazol-5-amine (250 mg, 1.72 mmol) in CH₂Br₂ (5 mL) was added TMSBr (1.05 g, 6.89 mmol) and tert-butyl nitrite (533 mg, 5.17 mmol) and the mixture stirred at 0° C. for 3 h under N₂. The mixture was concentrated and the residue purified by chromatography on silica gel (EtOAc) to give 5-bromo-3-(methoxymethyl)-1,2,4-thiadiazole as a yellow solid (50 mg, 14%). ¹H NMR (500 MHz, CDCl₃) δ ppm 4.71 (s, 2H), 3.51 (s, 3H).

### Preparation 313

### 4-bromo-2-(4-methylpiperazin-1-yl)thiazole

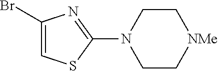 [SMILES: CN1CCN(c2nc(Br)cs2)CC1]

To a solution of 2,4-dibromothiazole (1 g, 4.12 mmol) in THF (10.0 mL) was added 1-methylpiperazine (2.5 g, 24.7 mmol) and TEA (2.5 g, 24.70 mmol) and the reaction mixture stirred at 90° C. for 12 h. The mixture was concentrated under reduced pressure and the residue purified by silica gel column chromatography (10-20% MeOH/DCM) to give 4-bromo-2-(4-methylpiperazin-1-yl)thiazole as a yellow oil (1.0 g, 93%). LCMS m/z=262.0 [M+H]⁺.

### Preparation 314

### 4-bromo-N-(2-methoxyethyl)-N-methylthiazol-2-amine

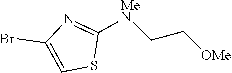 [SMILES: COCCN(C)c1nc(Br)cs1]

The title compound was prepared as a yellow oil (1.01 g, 93%) from 2,4-dibromothiazole and 2-methoxy-N-methylethan-1-amine using an analogous method to that described for Preparation 313. LCMS m/z=252.9 [M+H]⁺.

### Preparation 315

### 5-bromo-N-(2-methoxyethyl)-N-methyl-1,3,4-thiadiazol-2-amine

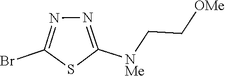 [SMILES: COCCN(C)c1nnc(Br)s1]

A solution of 2,5-dibromo-1,3,4-thiadiazole (400 mg, 1.64 mmol) and 2-methoxy-N-methylethan-1-amine (146 mg, 1.64 mmol) in DMF (8 mL) was added K₂CO₃ (680 mg, 4.92 mmol) and the mixture stirred at 80° C. for 4 h. The mixture was concentrated and the residue purified by chromatography on silica gel (1-25% EtOAc/PE) to give 5-bromo-N-(2-methoxyethyl)-N-methyl-1,3,4-thiadiazol-2-amine as a yellow oil (280 mg, 61%). LCMS m/z=252.0 [M+H]⁺.

### Preparation 316

### 2-bromo-5-(4-methylpiperazin-1-yl)-1,3,4-thiadiazole

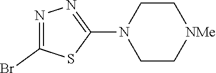 [SMILES: CN1CCN(c2nnc(Br)s2)CC1]

The title compound was prepared from 2,5-dibromo-1,3,4-thiadiazole and 1-methylpiperazine using an analogous method to that described for 5-bromo-N-(2-methoxyethyl)-N-methyl-1,3,4-thiadiazol-2-amine (Preparation 315). LCMS m/z=265.0 [M+H]⁺.

### Preparation 317

### 1-(2-bromothiazol-5-yl)-N,N-dimethylmethanamine

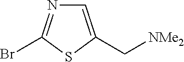

To a solution of 2-bromothiazole-5-carbaldehyde (200 mg, 1.04 mmol) in DCM (10 mL) was added Me₂NH·HCl (169.9 mg, 2.08 mmol) and the solution stirred at 25° C. for 1 h before NaBH(OAc)₃ (441.5 mg, 2.08 mmol) was added and stirring continued at 25° C. for 16 h. The mixture was concentrated and purified by prep-HPLC (Boston Prime C18 150×30 mm, 5 mm; 25-55% MeCN/H₂O (NH₄OH+NH₄HCO₃)) to give 1-(2-bromothiazol-5-yl)-N,N-dimethylmethanamine as a yellow solid (66.0 mg, crude). LCMS m/z=222.9 [M+H]⁺.

### Preparation 318 to 325

The title compounds were prepared from the appropriate aldehyde and amine using a similar method to that described for 1-(2-bromothiazol-5-yl)-N,N-dimethylmethanamine (Preparation 317)

| Prep | Name, Structure, Starting materials (SM), Data |
|---|---|
| 318 | 2-bromo-5-((4-methylpiperazin-1-yl)methyl)thiazole |
| | 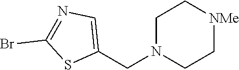 [SMILES: CN1CCN(Cc2cnc(Br)s2)CC1] |
| | SM: 2-bromothiazole-5-carbaldehyde; 1-methylpiperazine |
| | LCMS m/z = 276.1 [M + H]⁺. |
| 319 | 1-(2-bromothiazol-4-yl)-N,N-dimethylmethanamine |
| | 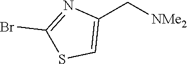 |
| | SM: 2-bromothiazole-4-carbaldehyde, Me₂NH.HCl |
| | ¹H NMR (500 MHz, CDCl₃) δ ppm 7.69 (s, 1H), 4.19 (s, 2H), 2.72 (s, |
| | 6H). |
| 320 | 2-bromo-4-((4-methylpiperazin-1-yl)methyl)thiazole |
| | 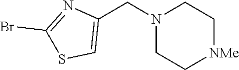 [SMILES: CN1CCN(Cc2csc(Br)n2)CC1] |
| | SM: 2-bromothiazole-4-carbaldehyde; 1-methylpiperazine |
| | LCMS m/z = 276.0 [M + H]⁺. |
| 321 | 1-(4-bromothiazol-2-yl)-N,N-dimethylmethanamine |
| | 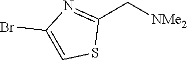 |
| | SM: 4-bromothiazole-2-carbaldehyde; Me₂NH.HCl |
| | LCMS m/z = 222.9 [M + H]⁺. |
| 322 | 4-bromo-2-((4-methylpiperazin-1-yl)methyl)thiazole |
| | 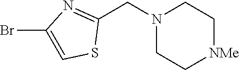 [SMILES: CN1CCN(Cc2nc(Br)cs2)CC1] |
| | SM: 4-bromothiazole-2-carbaldehyde; 1-methylpiperazine |
| | LCMS m/z = 221.0 [M + H]⁺. |
| 323 | 2-chloro-5-(pyrrolidin-1-ylmethyl)pyrazine |
| | 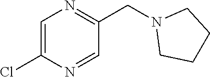 [SMILES: Clc1cnc(CN2CCCC2)cn1] |
| | SM: 5-chloropyrazine-2-carbaldehyde; pyrrolidine |
| | LCMS m/z = 198.1 [M + H]⁺. |
| 324 | 2-chloro-5-((4-methylpiperazin-1-yl)methyl)pyrazine |
| | 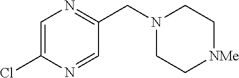 [SMILES: CN1CCN(Cc2cnc(Cl)cn2)CC1] |
| | SM: 5-chloropyrazine-2-carbaldehyde; 1-methylpiperazine |
| | LCMS m/z = 227.1 [M + H]⁺. |
| 325 | 4-((5-chloropyrazin-2-yl)methyl)morpholine |
| | 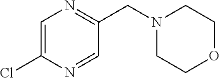 [SMILES: Clc1cnc(CN2CCOCC2)cn1] |
| | SM: 5-chloropyrazine-2-carbaldehyde; morpholine |
| | LCMS m/z = 214.1 [M + H]⁺. |

### Preparation 326

### 2-(azetidin-1-yl)-4-bromothiazole

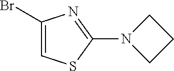

To a solution of azetidine (1 g, 4.12 mmol) in THF (10 mL) was added 2,4-dibromothiazole (1.41 g, 24.70 mmol, 1.66 mL) and TEA (2.50 g, 24.7 mmol) and the mixture stirred at 80° C. for 12 h. The mixture was diluted with H₂O (20 ml), extracted with EtOAc (20 ml×3), dried (Na₂SO₄) and evaporated to dryness under reduced pressure. The residue was purified by silica column chromatography (1-20% EtOAc/PE) to give 2-(azetidin-1-yl)-4-bromothiazole as a white solid (469 mg, 52%). LCMS m/z=221.0 [M+H]⁺.

### Preparation 327

### 6-(4-bromothiazol-2-yl)-2-oxa-6-azaspiro[3.3]heptane

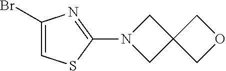 [SMILES: Brc1csc(N2CC3(COC3)C2)n1]

The title compound was prepared using an analogous method to that described for Preparation 326, from 2,4-dibromothiazole and 2-oxa-6-azaspiro[3.3]heptane. LCMS m/z=262.0 [M+H]⁺.

### Preparation 328

### 4-(2-(4-bromo-2H-1,2,3-triazol-2-yl)ethyl)morpholine

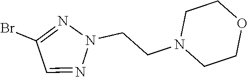 [SMILES: Brc1cnn(CCN2CCOCC2)n1]

To a solution of 4-bromo-2H-1,2,3-triazole (300 mg, 2.03 mmol) in DMF (10 mL) was added 4-(2-bromoethyl)morpholine (393.5 mg, 2.03 mmol), Cs₂CO₃ (1.3 g, 4.06 mmol) and the reaction stirred at 90° C. for 3 h. The mixture was concentrated and H₂O (20 mL) added and extracted with EtOAc (20 mL×3). The combined organics were washed with brine (20 mL), dried (Na₂SO₄) and concentrated. The residue was purified on silica gel column chromatography (15-33% EtOAc/PE) to give 4-(2-(4-bromo-2H-1,2,3-triazol-2-yl)ethyl)morpholine as a yellow oil (400 mg, 75%). ¹H NMR (400 MHz, CDCl₃) δ ppm 7.53 (s, 1H), 4.55 (s, 2H), 3.68 (br s, 4H), 2.99 (s, 2H), 2.51 (br s, 4H).

### Preparation 329

### 1-(2-(4-bromo-2H-1,2,3-triazol-2-yl)ethyl)-4-methylpiperazine

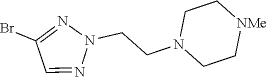 [SMILES: CN1CCN(CCn2ncc(Br)n2)CC1]

1-(2-(4-Bromo-2H-1,2,3-triazol-2-yl)ethyl)-4-methylpiperazine was prepared using an analogous method to that described for Preparation 328, from 4-bromo-2H-1,2,3-triazole. LCMS m/z=274.0 [M+H]⁺.

### Preparation 330

### 6-bromo-2,3-dihydroimidazo[2,1-b]oxazole

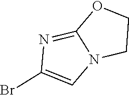

Part A: To a solution of 2,4,5-tribromo-1H-imidazole (1.0 g, 3.28 mmol) in DMF (10.0 mL) was added (2-bromoethoxy)(tert-butyl)dimethylsilane (942 mg, 3.94 mmol) and Cs₂CO₃ (3.2 g, 9.84 mmol) and the resulting mixture stirred at 70° C. for 16 h. The mixture diluted with water (60 mL) and extracted with EtOAc (50 mL×3). The combined organics were washed with brine (50 mL), dried (Na₂SO₄) and concentrated. The residue was purified by chromatography on silica gel (5-20% EtOAc/PE) to give 2,4,5-tribromo-1-(2-((tert-butyldimethylsilyl)oxy)ethyl)-1H-imidazole as a yellow oil (1.3 g, 86%).

Part B: To a solution of 2,4,5-tribromo-1-(2-((tert-butyldimethylsilyl)oxy)ethyl)-1H-imidazole (1.3 g, 2.81 mmol) in dioxane (8.0 mL) was added HCl/dioxane (4 M, 8.0 mL) and the resulting mixture stirred at 25° C. for 1 h. The reaction mixture was concentrated under reduced pressure to give 2-(2,4,5-tribromo-1H-imidazol-1-yl)ethan-1-ol as a white solid (1.0 g, crude).

Part C: To a solution of 2-(2,4,5-tribromo-1H-imidazol-1-yl)ethan-1-ol (1.0 g, 2.87 mmol) in DMF (8.0 mL) was added NaH (229 mg, 5.73 mmol, 60% purity) at 0° C. and the reaction stirred at 0° C. for 2 h. The reaction was quenched with NH₄Cl aq. (20 mL), diluted with water (30 mL) and extracted with EtOAc (30 mL×3). The combined organics were washed with brine (30 mL), dried (Na₂SO₄) and concentrated to give 5,6-dibromo-2,3-dihydroimidazo[2,1-b]oxazole as yellow oil (580 mg, 75%) as yellow oil.

Part D: To a solution of 5,6-dibromo-2,3-dihydroimidazo[2,1-b]oxazole (580 mg, 2.16 mmol) in THF (8.0 mL) was added n-BuLi (2.5 M, 2.16 mmol, 0.86 mL) slowly at −70° C. under N₂ and the resulting mixture stirred at −70° C. for 1 h under N₂. The reaction was quenched with NH₄Cl (sat, 20 mL), diluted with H₂O (30 mL) and extracted with EtOAc (30 mL×3). The combined organics was dried (Na₂SO₄), concentrated and the residue purified by chromatography on silica gel (5-25% EtOAc/PE) to give 6-bromo-2,3-dihydroimidazo[2,1-b]oxazole as a white solid (190 mg, 46%). ¹H NMR (400 MHz, CDCl₃) δ ppm: 6.60 (s, 1H), 5.00 (d, J=7.6 Hz, 2H), 4.15 (d, J=8.0 Hz, 2H).

### Preparation 331

### 2-bromo-5-methyl-4,5,6,7-tetrahydropyrazolo[1,5-a]pyrazine

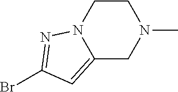

To a solution of 2-bromo-4,5,6,7-tetrahydropyrazolo[1,5-a]pyrazine (230 mg, 1.14 mmol) in MeOH (1 mL) was added NaBH₃CN (357.7 mg, 5.69 mmol), (CH₂O)n (2.73 g, 2.28 mmol) and the mixture was stirred at 25° C. for 16 h. The mixture was concentrated and purified by Prep-HPLC (Method R, Gradient 12-42%) to give 2-bromo-5-methyl-4,5,6,7-tetrahydropyrazolo[1,5-a]pyrazine (135 mg, 54.9% yield) as a white solid. LCMS m/z=218.0 [M+H]⁺

### Preparation 332

### 2-bromo-7-methyl-5,6,7,8-tetrahydroimidazo[1,2-a]pyrazine

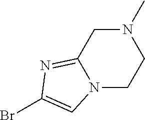

2-bromo-7-methyl-5,6,7,8-tetrahydroimidazo[1,2-a]pyrazine was obtained as a white solid, 120 mg, 46.8%, from 2-bromo-5,6,7,8-tetrahydroimidazo[1,2-a]pyrazine and (CH2O)n, following the procedure described in Preparation 331. LCMS m/z=218.0 [M+H]⁺

### Preparation 333

### 2-bromo-4,5,6,7-tetrahydropyrazolo[1,5-a]pyrimidine

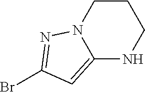

To a solution of 3-bromo-1H-pyrazol-5-amine (900 mg, 5.56 mmol) in dioxane (10 mL) was added 1,3-dibromopropane (1.4 g, 6.67 mmol) and DIPEA (2.2 g, 16.67 mmol). The resulting solution was heated at 120° C. for 12 h in a sealed tube. The mixture was concentrated and purified by Prep-HPLC (Method B, 15-35%) to give 2-bromo-4,5,6,7-tetrahydropyrazolo[1,5-a]pyrimidine (328 mg, 29.2% yield) as a white solid. LCMS m/z=202.0 [M+H]⁺

### Preparation 334

### 2-bromo-4-methyl-4,5,6,7-tetrahydropyrazolo[1,5-a]pyrimidine

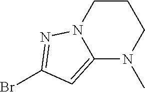

2-Bromo-4-methyl-4,5,6,7-tetrahydropyrazolo[1,5-a]pyrimidine was obtained as a yellow oil, 100 mg, 46.8% from 2-bromo-4,5,6,7-tetrahydropyrazolo[1,5-a]pyrimidine (Preparation 333), following the procedure described in Preparation 293. LCMS m/z=218.0 [M+H]⁺

### Preparation 335

### 2-bromo-5-(methyl-d3)-4,5,6,7-tetrahydropyrazolo[1,5-a]pyrazine

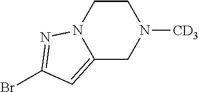 [SMILES: [2H]C([2H])([2H])N1CCn2nc(Br)cc2C1]

A vial containing 2-bromo-4,5,6,7-tetrahydropyrazolo[1,5-a]pyrazine trifluoroacetate (317 mg, 1.00 mmol) in anhydrous 2-MeTHF (4 mL) was cooled in an ice water bath, then DIPEA (0.9 mL, 5.2 mmol) and NaOtBu (299 mg, 3.1 mmol) were added under N₂ at <5° C. After 20 minutes, CD₃I (0.2 mL, 3.2 mmol) was added and the reaction was maintained at <5° C. for 1.5 h. The reaction was quenched by slow addition of solid NaHCO₃, the mixture was stirred at 23° C. for 1 h, then was concentrated under reduced pressure. The residue was loaded onto a silica gel column and purified with (50-100% 3:1 EtOAc: EtOH in heptane) to afford 2-bromo-5-(methyl-d3)-4,5,6,7-tetrahydropyrazolo[1,5-a]pyrazine (110 mg, crude). LCMS m/z=219.0 [M+H]⁺.

### Preparation 336

### 2-Iodo-4,5,6,7-tetrahydropyrazolo[1,5-a]pyrazine hydrochloride

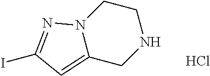

To a flask containing tert-butyl 2-iodo-6,7-dihydro-4H-pyrazolo[1,5-a]pyrazine-5-carboxylate (938 mg, 2.69 mmol) in DCM (11 mL) was added HCl in dioxane (4M solution) (2 mL, 8.00 mmol). Upon complete addition of HCl, the reaction was stirred at 23° C. for 72 h. DCM (11 mL) then HCl in dioxane (4 mL, 16.00 mmol) were added and the mixture stirred for 45 h then concentrated in vacuo. The crude product was diluted with EtOAc then sonicated for 5 mins. The heterogeneous mixture was filtered to afford a faint yellow white solid as 2-iodo-4,5,6,7-tetrahydropyrazolo[1,5-a]pyrazine (HCl Salt) (703 mg, crude). LCMS m/z=249.9 [M+H]⁺.

### Preparation 337

### 1-(2-iodo-6,7-dihydropyrazolo[1,5-a]pyrazin-5(4H)-yl)ethan-1-one

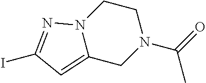

To a vial containing 2-iodo-4,5,6,7-tetrahydropyrazolo[1,5-a]pyrazine hydrochloride (Preparation 336, 290 mg, 1.02 mmol) in DCM (5 mL) was added DIPEA (1.1 mL, 6.32 mmol) and the solution stirred for 15 mins. Acetic anhydride (0.2 mL, 2.12 mmol) was added dropwise at 23° C. and the reaction stirred for 3 h at this temperature. The reaction was diluted with DCM, then washed with saturated aqueous NaHCO₃ and the layers separated. The aqueous layer was extracted with DCM (3×) and the combined organic extracts dried over MgSO₄, filtered and evaporated under reduced pressure to give 1-(2-iodo-6,7-dihydropyrazolo[1,5-a]pyrazin-5(4H)-yl)ethan-1-one (287 mg, 97% yield). LCMS m/z=291.9 [M+H]⁺.

### Preparation 338

### Methyl 3-bromo-1-(2-((tert-butoxycarbonyl)amino)ethyl)-1H-pyrazole-5-carboxylate

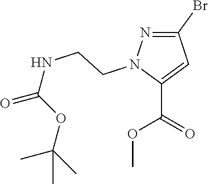 [SMILES: COC(=O)c1cc(Br)nn1CCNC(=O)OC(C)(C)C]

A vial containing tert-butyl N-(2-hydroxyethyl)carbamate (488 mg, 3.03 mmol) and PPh₃ (812 mg, 3.1 mmol) in anhydrous THF (10 mL) was degassed then backfilled with N₂ and cooled in ice water bath. DEAD (2.1 mL, 5.33 mmol, 40% purity) was added dropwise at <5° C., the solution allowed to warm to rt, then methyl 3-bromo-1H-pyrazole-5-carboxylate (507 mg, 2.5 mmol) was added in portions The reaction mixture was stirred at rt for 19 h, then diluted with heptane, the mixture stirred for 1 h and filtered. The filtrate was concentrated under reduced pressure and the residue was purified by silica gel column chromatography eluting with (5 to 55% EtOAc in heptane) to give methyl 3-bromo-1-(2-((tert-butoxycarbonyl)amino)ethyl)-1H-pyrazole-5-carboxylate (537 mg, crude). LCMS m/z=347.9 [M+H]⁺.

### Preparation 339

### 1-(2-aminoethyl)-3-bromo-1H-pyrazole-5-carboxylic acid

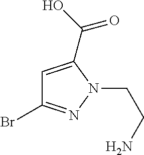 [SMILES: NCCn1nc(Br)cc1C(=O)O]

To a solution of methyl 5-bromo-1H-pyrazole-3-carboxylate (3 g, 14.63 mmol) in dry DMF (50 mL) at 0° C. was added NaH (1.17 g, 29.27 mmol, 60% purity) and the mixture was stirred at 0° C. for 15 min. tert-Butyl 2,2-dioxooxathiazolidine-3-carboxylate (3.92 g, 17.56 mmol) was added and the reaction was stirred at rt for 1 h. The mixture was cooled to 0° C., slowly quenched with water, then extracted with EtOAc (3×). The aqueous layer was concentrated in vacuo, the residue was taken up in MeOH, filtered with aid of MeOH and concentrated in vacuum to give 2-(2-aminoethyl)-5-bromo-pyrazole-3-carboxylic acid. LCMS m/z=234.0 [M+H]⁺

### Preparation 340

### 3-bromo-1-(2-((tert-butoxycarbonyl)amino)ethyl)-1H-pyrazole-5-carboxylic acid

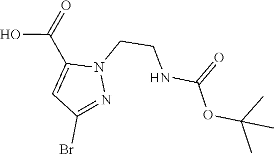 [SMILES: CC(C)(C)OC(=O)NCCn1nc(Br)cc1C(=O)O]

NaOH (512.7 mg, 12.82 mmol) and Boc anhydride (4.20 g, 19.23 mmol) were added to a solution of 2-(2-aminoethyl)-5-bromo-pyrazole-3-carboxylic acid (Preparation 339, 3 g, 12.82 mmol) in water (25 mL) and dioxane (25 mL) and the resulting mixture was stirred at rt for 17 h. The reaction was concentrated in vacuo, acidified to pH=3 and extracted with EtOAc (3×). The combined organic extracts were washed with brine, dried (MgSO₄), filtered and concentrated in vacuo to give 3-bromo-1-(2-((tert-butoxycarbonyl)amino)ethyl)-1H-pyrazole-5-carboxylic acid (2.71 g, 63.3% yield) as a white solid. LCMS m/z=334.0 [M+H]⁺

### Preparation 341

### tert-butyl (2-(3-bromo-5-(methoxy(methyl)carbamoyl)-1H-pyrazol-1-yl)ethyl)carbamate

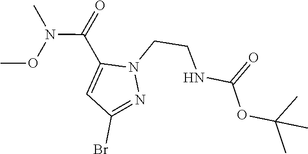 [SMILES: CON(C)C(=O)c1cc(Br)nn1CCNC(=O)OC(C)(C)C]

To crude 3-bromo-1-(2-((tert-butoxycarbonyl)amino)ethyl)-1H-pyrazole-5-carboxylic acid (Preparation 340, 2.7 g, 8.08 mmol) in dry DMF (15 mL) was added TEA (2.45 g, 24.24 mmol), N-methoxymethanamine HCl (788.1 mg, 8.08 mmol) and HATU (3.07 g, 8.08 mmol). The resulting suspension was stirred at rt for 3 h. The reaction was quenched with water and extracted with EtOAc (3×) and the combined organic extracts were washed with brine (3×), dried (MgSO₄), filtered and concentrated in vacuo. The residue was purified by silica gel chromatography (0-100% EtOAc in Hept) to give tert-butyl (2-(3-bromo-5-(methoxy(methyl)carbamoyl)-1H-pyrazol-1-yl)ethyl)carbamate (2.18 g, 71.5% yield) was obtained as a white solid. LCMS m/z=399.2 [M+Na]+

### Preparation 342

### tert-butyl (2-(5-acetyl-3-bromo-1H-pyrazol-1-yl)ethyl)carbamate

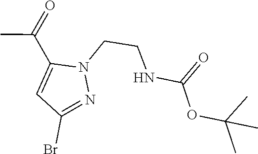 [SMILES: CC(=O)c1cc(Br)nn1CCNC(=O)OC(C)(C)C]

To a solution of tert-butyl (2-(3-bromo-5-(methoxy(methyl)carbamoyl)-1H-pyrazol-1-yl)ethyl)carbamate (Preparation 341, 2.15 g, 5.70 mmol) in dry THF (20 mL) under N₂ was added a solution of MeMgBr (3 M, 7.60 mL) in 2-THF, dropwise at 0° C. The resulting solution was stirred at rt overnight. The mixture was cooled to 0° C. and was quenched with NH₄Cl solution and extracted with EtOAc (2×). The combined organic extracts were washed with brine, dried over MgSO₄ and filtered. The filtrate was concentrated in vacuo and the residue was purified via silica gel column chromatography (PE/EtOAc 0-60%) to give tert-butyl (2-(5-acetyl-3-bromo-1H-pyrazol-1-yl)ethyl)carbamate (1.35 g, 71.3% yield) as a white solid. LCMS m/z=332.1 [M+H]+

### Preparation 343

### Methyl 1-(2-aminoethyl)-3-bromo-1H-pyrazole-5-carboxylate

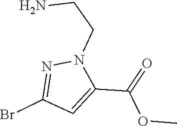

To a vial containing methyl 3-bromo-1-(2-((tert-butoxycarbonyl)amino)ethyl)-1H-pyrazole-5-carboxylate (Preparation 342, 537 mg, 1.54 mmol) in DCM (8 mL) was added TFA (1 mL, 13 mmol) and the reaction was stirred at 23° C. for 3.5 h. The mixture was diluted with DCM then neutralised by addition of 1.5 M aq. K₂CO₃ solution. The phases were separated, the organic dried over MgSO₄, filtered and the filtrate evaporated under reduced pressure to give methyl 1-(2-aminoethyl)-3-bromo-1H-pyrazole-5-carboxylate as a white solid (414 mg, crude). LCMS m/z=248.0 [M+H]⁺.

### Preparation 344

### 2-Bromo-6,7-dihydropyrazolo[1,5-a]pyrazin-4(5H)-one

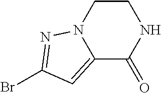 [SMILES: O=C1NCCn2nc(Br)cc21]

To a vial containing methyl 1-(2-aminoethyl)-3-bromo-1H-pyrazole-5-carboxylate (Preparation 343, 382 mg, 1.54 mmol) in anhydrous 2-MeTHF (12 mL) was added NaOtBu (303 mg, 3.16 mmol) under N₂ and the homogeneous reaction was heated to 45° C. and stirred for 19 h. The mixture was cooled to 23° C. then diluted with water and EtOAc. The mixture was neutralized by dropwise addition of aq. 2 N HCl, the layers separated and the aqueous layer extracted with EtOAc (3×). The combined organic extracts were dried over anhydrous Na₂SO₄, filtered and evaporated under reduced pressure to afford 2-bromo-6,7-dihydropyrazolo[1,5-a]pyrazin-4(5H)-one (349 mg, crude). LCMS m/z=215.9 [M+H]⁺.

### Preparation 345

### 2-bromo-4-methyl-4,5,6,7-tetrahydropyrazolo[1,5-a]pyrazine

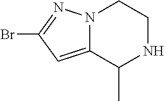

To tert-butyl (2-(5-acetyl-3-bromo-1H-pyrazol-1-yl)ethyl)carbamate (Preparation 342, 1.35 g, 4.06 mmol) was added MeOH (1 mL) followed by HCl (4 M, 36.58 mmol, 9.14 mL) solution in dioxane and the resulting mixture was stirred at rt for 1 h. The solvents were removed in vacuo. To the solid residue was added TEA (4.11 g, 40.64 mmol) followed by NaBH₃CN (1 M, 16.26 mmol, 16.26 mL) solution in THF and the resulting slurry was stirred at rt overnight. The reaction mixture was concentrated in vacuo and purified by silica gel chromatography (DCM/MeOH 0-5%) to obtain 2-bromo-4-methyl-4,5,6,7-tetrahydropyrazolo[1,5-a]pyrazine (870 mg, crude) as a colorless oil. LCMS m/z=216.0 [M+H]+

### Preparation 346

### 2-bromo-4,5-dimethyl-4,5,6,7-tetrahydropyrazolo[1,5-a]pyrazine

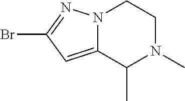

To 2-bromo-4-methyl-4,5,6,7-tetrahydropyrazolo[1,5-a]pyrazine (Preparation 345, 400 mg, 1.58 mmol) was added a solution of formaldehyde (7.70 g, 2.38 mmol, 37% purity) in water followed by NaBH₃CN (1 M, 4.75 mmol, 4.75 mL) in THF. The resulting mixture was stirred at 25° C. for 16 h, then concentrated in vacuo. The residue was purified by silica gel chromatography (0-10% MeOH in DCM) to give 2-bromo-4,5-dimethyl-6,7-dihydro-4H-pyrazolo[1,5-a]pyrazine (316 mg, 86.7% yield) as a white solid. LCMS m/z=230.1 [M+H]+

### Preparation 347

### 2-bromo-6-methyl-4,5,6,7-tetrahydropyrazolo[1,5-a]pyrazine

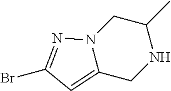

A suspension of 2-bromo-6-methyl-6,7-dihydro-5H-pyrazolo[1,5-a]pyrazin-4-one (commercial, 2.50 g, 10.87 mmol) in BH₃-THF (1 M, 108.7 mmol, 108.7 mL) was stirred at 80° C. for 16 h under N₂. MeOH (20 mL) was added and the mixture stirred at 80° C. for 1 h. The mixture was concentrated under vacuum to give 2-bromo-6-methyl-4,5,6,7-tetrahydropyrazolo[1,5-a]pyrazine as a yellow gum, LCMS m/z=216.0 [M+H]⁺

### Preparation 348

### 2-bromo-5,6-dimethyl-4,5,6,7-tetrahydropyrazolo[1,5-a]pyrazine

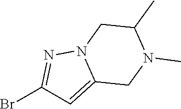

2-Bromo-5,6-dimethyl-4,5,6,7-tetrahydropyrazolo[1,5-a]pyrazine was obtained as a white solid, 410 mg, 19%, from 2-bromo-6-methyl-4,5,6,7-tetrahydropyrazolo[1,5-a]pyrazine (Preparation 347) and formaldehyde, following a similar method to that described in Preparation 346. LCMS m/z=230.1 [M+H]⁺

### Preparation 349

### N-((3-Bromo-1H-pyrazol-5-yl)methyl)-2-chloro-2-fluoro-N-methylethan-1-amine

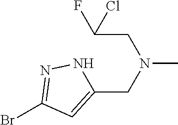

To a flask containing 2-chloro-2-fluoro-N-methyl-ethanamine hydrochloride (165 mg, 1.11 mmol) and 3-bromo-5-(chloromethyl)-1H-pyrazole hydrochloride (207 mg, 0.894 mmol) in anhydrous DMF (5 mL) was added DIPEA (1.1 mL, 6.32 mmol) under N₂ and the reaction was stirred at 23° C. for 0.5 h, then at 40° C. for 14 h. The mixture was cooled to 23° C., K₂CO₃ (333 mg, 2.4 mmol) was added and the mixture was heated to 65° C. for 6 days. The mixture was cooled to 23° C. then diluted with EtOAc. After filtration through a syringe filter, the filtrate was evaporated under reduced pressure to give N-((3-bromo-1H-pyrazol-5-yl)methyl)-2-chloro-2-fluoro-N-methylethan-1-amine (242 mg, crude).

### Preparation 350

### 2-Bromo-7-fluoro-5-methyl-4,5,6,7-tetrahydropyrazolo[1,5-a]pyrazine

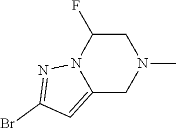

To a vial containing N-((3-bromo-1H-pyrazol-5-yl)methyl)-2-chloro-2-fluoro-N-methylethan-1-amine (Preparation 349, 242 mg, 0.894 mmol) in anhydrous DMF (2 mL) was added Cs₂CO₃ (1.2 g, 3.7 mmol) and the reaction mixture stirred at 45° C. for 3 h. The mixture was diluted with EtOAc then filtered through a syringe filter. The filtrate was evaporated under reduced pressure to give 2-bromo-7-fluoro-5-methyl-4,5,6,7-tetrahydropyrazolo[1,5-a]pyrazine (209 mg, crude). LCMS m/z=233.9 [M+H]⁺.

### Preparation 351

### 2-bromo-7-methyl-5,6,7,8-tetrahydro-[1,2,4]triazolo[1,5-a]pyrazine

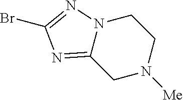 [SMILES: CN1CCn2nc(Br)nc2C1]

Part A: LiBH₄ (333 mg, 15.3 mmol) was added to 2-bromo-[1,2,4]triazolo[1,5-a]pyrazine (760 mg, 3.82 mmol) in EtOH (10 mL) at 0° C. and the temperature was slowly raised to 50° C. and stirred for 12 h. The reaction mixture was concentrated to dryness under reduced pressure and the residue adjusted to pH 2-3 under ice-cooling using aqueous HCl solution and washed with EtOAc. The aqueous solution was adjusted to pH 9-10 with Na₂CO₃ and extracted into DCM (30 mL×3), washed with brine (3×30 mL), dried (Na₂SO₄) and evaporated to dryness to give 2-bromo-5,6,7,8-tetrahydro-[1,2,4]triazolo[1,5-a]pyrazine as a white solid (450 mg, crude).

Part B: Paraformaldehyde (13.3 g, 11.1 mmol) was added to a solution of 2-bromo-5,6,7,8-tetrahydro-[1,2,4]triazolo[1,5-a]pyrazine (450 mg, 2.22 mmol) in MeOH (5.0 mL) followed by NaBH₃CN (696 mg, 11.1 mmol) and the reaction stirred at 25° C. for 2 h. The mixture was concentrated and the residue purified by prep-HPLC (Method Z, Gradient 0-10%) to afford 2-bromo-7-methyl-5,6,7,8-tetrahydro-[1,2,4]triazolo[1,5-a]pyrazine as a white solid (210 mg, 44%). LCMS m/z=218.0 [M+H]⁺.

### Preparation 352

### (3-bromo-1-(2-(tert-butyldimethylsilyl)ethyl)-1H-1,2,4-triazol-5-yl)methyl 4-methylbenzenesulfonate

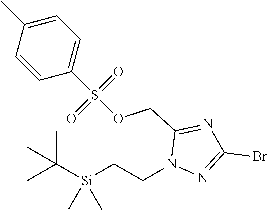 [SMILES: Cc1ccc(S(=O)(=O)OCc2nc(Br)nn2CC[Si](C)(C)C(C)(C)C)cc1]

Part A: To a solution of methyl 3-bromo-1H-1,2,4-triazole-5-carboxylate (3.0 g, 14.6 mmol) in DMF (30 mL) was added (2-bromoethoxy)(tert-butyl)dimethylsilane (6.9 g, 29.1 mmol) and Cs₂CO₃ (9.5 g, 29.1 mmol) and the resulting mixture stirred at 20° C. for 3 days. The mixture was added to water (60 mL) and extracted with EtOAc (50 mL×3). The combined organics were washed with brine (50 mL×2), dried (Na₂SO₄) and concentrated. The residue was purified by chromatography on silica gel (5-20% EtOAc/PE) to give methyl 3-bromo-1-(2-(tert-butyldimethylsilyl)ethyl)-1H-1,2,4-triazole-5-carboxylate as a colourless oil (1.7 g, 32%).

Part B: To a solution of methyl 3-bromo-1-(2-(tert-butyldimethylsilyl)ethyl)-1H-1,2,4-triazole-5-carboxylate (1.6 g, 4.39 mmol) in THF (20 mL) was added LiBH₄ (2 M, 17.57 mmol, 8.78 mL) slowly at 0° C. and the reaction stirred at 25° C. for 1 h. The mixture was quenched with sat. NH₄Cl (30 mL), poured into H₂O (30 mL), extracted with EtOAc (30 mL×3) and washed with brine (30 mL×2). The combined organics was dried over anhydrous Na₂SO₄, filtered and concentrated in vacuo. The residue was purified by chromatography on silica gel (5-25% EtOAc/PE) to give (3-bromo-1-(2-(tert-butyldimethylsilyl)ethyl)-1H-1,2,4-triazol-5-yl)methanol as a white solid (1.1 g, 74%).

Part C: To a solution of (3-bromo-1-(2-(tert-butyldimethylsilyl)ethyl)-1H-1,2,4-triazol-5-yl)methanol (500 mg, 1.49 mmol) in DCM (5 mL) was added TsCl (340 mg, 1.78 mmol), DMAP (9.1 mg, 0.074 mmol) and TEA (752 mg, 7.43 mmol) at 0° C. The mixture was stirred at 20° C. for 2 h, poured into water (30 mL) and extracted with DCM (20 mL×3). The combined organics were washed with brine (20 mL×2), dried over Na₂SO₄, filtered and concentrated and the residue purified by chromatography on silica gel (5-20% EtOAc/PE) to give (3-bromo-1-(2-(tert-butyldimethylsilyl)ethyl)-1H-1,2,4-triazol-5-yl)methyl 4-methylbenzenesulfonate as a white solid (300 mg, 41%). ¹H NMR (400 MHz, CDCl₃) δ ppm: 7.74 (d, J=8.0 Hz, 2H), 7.34 (d, J=8.0 Hz, 2H), 5.18 (s, 2H), 4.26 (t, J=4.8 Hz, 2H), 3.88 (t, J=4.8 Hz, 2H), 2.46 (s, 3H), 0.78 (s, 9H), −0.08 (s, 6H).

### Preparation 353

### 2-bromo-5,6-dihydro-8H-[1,2,4]triazolo[5,1-c][1,4]oxazine

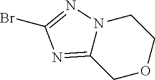

Part A: To a solution of (3-bromo-1-(2-(tert-butyldimethylsilyl)ethyl)-1H-1,2,4-triazol-5-yl)methyl 4-methylbenzenesulfonate (Preparation 352, 300 mg, 0.611 mmol) in dioxane (2 mL) was added HCl/dioxane (4 M, 2.0 mL) and the resulting mixture was stirred at 20° C. for 1 h. The reaction was concentrated under reduced pressure to give (3-bromo-1-(2-hydroxyethyl)-1H-1,2,4-triazol-5-yl)methyl 4-methylbenzenesulfonate as a colourless oil (220.5 mg, crude).

Part B: To a solution of (3-bromo-1-(2-hydroxyethyl)-1H-1,2,4-triazol-5-yl)methyl 4-methylbenzenesulfonate (220.5 mg, 0.59 mmol) in MeOH (3 mL) and EtOH (0.3 mL) was added NaOMe (63.3 mg, 1.17 mmol) and the reaction stirred at 60° C. for 2 h. The mixture was evaporated to dryness, added to water (20 mL) and extracted with EtOAc (20 mL×3). The combined organics were washed with brine (20 mL×2), dried over Na₂SO₄, filtered and concentrated to give 2-bromo-5,6-dihydro-8H-[1,2,4]triazolo[5,1-c][1,4]oxazine as a white solid (90 mg, crude). LCMS m/z=205.6 [M+H]⁺.

### Preparation 354

### 4-chloro-6-(4-methylpiperazin-1-yl)pyrimidine

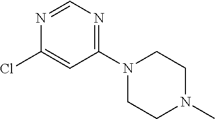

To a solution of 4,6-dichloropyrimidine (500 mg, 3.36 mmol) and 1-methylpiperazine (302.6 mg, 3.02 mmol) in EtOH (20 mL) was added TEA (373.6 mg, 3.69 mmol) at 0° C. The mixture was stirred at 20° C. for 5 h. The reaction mixture was diluted with EtOAc (100 mL) and concentrated under reduced pressure to give 4-chloro-6-(4-methylpiperazin-1-yl)pyrimidine (700 mg, crude) as a white solid. LCMS m/z=213.3 [M+H]⁺

### Preparation 355

### 2-bromo-6-(2-methoxyethoxy)pyrazine

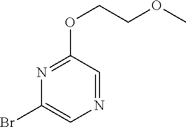

To a stirring solution of 2,6-dibromopyrazine (300 mg, 1.26 mmol) and 2-methoxyethanol (105.6 mg, 1.39 mmol) in anhydrous THF (12 mL) was added NaH (30.26 mg, 1.26 mmol) at 0° C. and the reaction was allowed to warm to rt and stirred for 3 h. The reaction mixture was quenched with NH₄Cl at 0° C. and was extracted with EtOAc. The solvent was evaporated and the crude product was purified by silica gel column chromatography (0-100% heptane-EtOAc) to yield 2-bromo-6-(2-methoxyethoxy)pyrazine (268 mg, 91.2% yield) as a clear liquid; ¹H NMR (500 MHz, DMSO-d₆) δ 8.41 (d, J=1.3 Hz, 1H), 8.37 (s, 1H), 4.42-4.40 (m, 2H), 3.69-3.66 (m, 2H), 3.29 (s, 3H).

### Preparation 356

### 2-bromo-6-(oxetan-3-yloxy)pyrazine

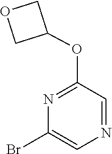 [SMILES: Brc1cncc(OC2COC2)n1]

2-Bromo-6-(oxetan-3-yloxy)pyrazine was obtained as a white solid, 217 mg, 74.5%, from 2,6-dibromopyrazine and oxetan-3-ol, following the method described in Preparation 355. ¹H NMR (500 MHz, CD₂Cl₂) δ 8.27 (d, J=1.9 Hz, 1H), 8.22 (d, J=1.9 Hz, 1H), 5.60 (p, J=5.6 Hz, 1H), 4.95 (t, J=7.0 Hz, 2H), 4.71-4.66 (m, 2H).

### Preparation 357

### 4-chloro-6-(2-methoxyethoxy)pyrimidine

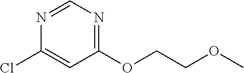

NaH (525.6 mg, 13.14 mmol, 60% purity) was dissolved in THF (10 mL), the solution stirred at 0° C. for 15 mins, then a solution of 2-methoxyethanol (1.0 g, 13.14 mmol) in THF (10 mL) was added dropwise. The mixture was warmed to 25° C. for 30 mins, then a solution of 4,6-dichloropyrimidine (3.1 g, 21.03 mmol) in THF (10 mL) was added dropwise and the reaction was stirred at 25° C. for 2 h. The mixture was diluted with H₂O (200 mL) and extracted with EtOAc (100 mL×3), dried over Na₂SO₄, filtered and concentrated under reduced pressure. The residue was purified by column chromatography (PE/EtOAc=100/1 to 3/1) to give 4-chloro-6-(2-methoxyethoxy)pyrimidine (1.8 g, 72.6% yield) as a colorless oil. ¹H NMR (400 MHz, CDCl₃) δ ppm: 8.54 (s, 1H), 6.80 (s, 1H), 4.52-4.55 (m, 2H), 3.69-3.72 (m, 2H), 3.40 (s, 3H).

### Preparation 358

### 6-chloro-3-2-methoxyethyl)pyrimidin-4(3H)-one

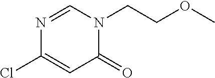

To a solution of 6-chloropyrimidin-4(3H)-one (500 mg, 3.83 mmol) and 1-bromo-2-methoxyethane (585.6 mg, 4.21 mmol) in DMF (15 mL) was added K₂CO₃ (1.1 g, 7.66 mmol) and the reaction mixture was stirred at 50° C. for 16 h. The mixture was diluted with H₂O (200 mL), extracted with EtOAc (100 mL×3), dried over Na₂SO₄, filtered and concentrated under reduced pressure. The residue was purified by prep-TLC (PE/EtOAc=1/1) to give 6-chloro-3-(2-methoxyethyl)pyrimidin-4(3H)-one (400.0 mg, 55.4% yield) as a white solid. LCMS m/z=189.2 [M+H]⁺

### Preparation 359

### 6-chloro-N-(2-methoxyethyl)pyrazin-2-amine

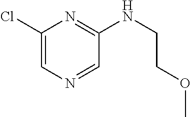

To a solution of 2,6-dichloropyrazine (500 mg, 3.36 mmol) in DMF (30 mL) was added 2-methoxyethan-1-amine (378.1 mg, 5.03 mmol) and Cs₂CO₃ (3.3 g, 10.07 mmol). The resulting mixture was stirred at 100° C. for 2 h. The mixture was quenched with H₂O (20 mL) and extracted with EtOAc (20 mL×2). The combined organic phase was washed with brine (20 mL×2), dried over Na₂SO₄ and filtered. The filtrate was concentrated under reduced pressure and the residue was purified by chromatography on silica gel (PE/EtOAc=1/1) to give 6-chloro-N-(2-methoxyethyl)pyrazin-2-amine (150 mg, 23.8% yield) as yellow oil. ¹H NMR (400 MHz, CDCl₃) δ ppm: 7.79 (s, 1H), 7.78 (s, 1H), 5.07 (br s, 1H), 3.54-3.60 (m, 4H), 3.39 (s, 3H).

### Preparation 360

### 6-chloro-N-(2-methoxyethyl)-N-methylpyrazin-2-amine

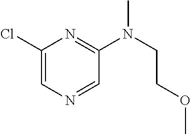

6-Chloro-N-(2-methoxyethyl)-N-methylpyrazin-2-amine was obtained as a yellow oil, 400 mg, 59.1% yield, from 2,6-dichloropyrazine and 2-methoxy-N-methylethan-1-amine, following the procedure described in Preparation 359. ¹H NMR (400 MHz, CDCl₃) δ ppm: 7.89 (s, 1H), 7.76 (s, 1H), 3.72 (t, J=5.2 Hz, 2H), 3.59 (t, J=5.2 Hz, 2H), 3.35 (s, 3H), 3.14 (s, 3H).

### Preparation 361

### 1,3-dioxoisoindolin-2-yl 1,4-dioxane-2-carboxylate

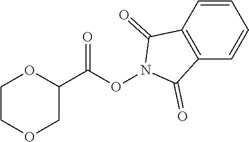 [SMILES: O=C(ON1C(=O)c2ccccc2C1=O)C1COCCO1]

A mixture of 1,4-dioxane-2-carboxylic acid (500 mg, 3.78 mmol), 2-hydroxyisoindoline-1,3-dione (625 mg, 3.83 mmol), and DMAP (50 mg, 0.409 mmol) in DCM (10 mL) was stirred at rt for 5 mins, then N,N′-diisopropylcarbodiimide (500 mg, 3.96 mmol) was added dropwise over 5 mins and the reaction was stirred for 2 h at rt. The reaction was filtered through a 0.2 um syringe filter and purified directly via silica gel chromatography (heptane to EtOAc) to give (1,3-dioxoisoindolin-2-yl) 1,4-dioxane-2-carboxylate (900 mg, 72.1% yield) as a white solid. ¹H NMR (500 MHz, DMSO-d₆) δ (ppm)=8.05-7.94 (m, 4H), 4.98 (br d, J=1.2 Hz, 1H), 3.97 (br d, J=3.7 Hz, 2H), 3.93-3.87 (m, 1H), 3.71 (br d, J=4.9 Hz, 3H).

### Preparation 362

### 2-chloro-5-(1,4-dioxan-2-yl)pyrazine

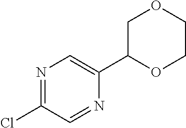 [SMILES: Clc1cnc(C2COCCO2)cn1]

A mixture of 2-bromo-5-chloro-pyrazine (251.3 mg, 1.30 mmol), (1,3-dioxoisoindolin-2-yl), 1,3-dioxoisoindolin-2-yl 1,4-dioxane-2-carboxylate (Preparation 361, 300 mg, 0.909 mmol), 2-(2-pyridyl)pyridine (30 mg, 0.192 mmol), AgNO₂ (80 mg, 0.471 mmol) and NiCl₂-dme (40 mg, 0.182 mmol) in DMF (6 mL) was electrolyzed on an IKA Electrasyn at 12 mA for 4 electron equivs. The reaction was diluted with EtOAc (15 mL), filtered through a 0.2 um syringe filter, and concentrated to dryness. The crude material was purified via silica gel chromatography (heptane to EtOAc) to give 2-chloro-5-(1,4-dioxan-2-yl)pyrazine as a white solid. (80 mg, 19.3% yield). LCMS m/z=201.0 [M+H]⁺.

### Preparation 363

### 3-methyl-6-(tributylstannyl)pyrimidin-4(3H)-one

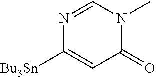

To a solution of 6-chloro-3-methylpyrimidin-4(3H)-one (1.0 g, 6.92 mmol) in toluene (10 mL) was added Pd₂(dba)₃ (633.5 mg, 0.691 mmol) and PCy₃ (485.0 mg, 1.73 mmol) at 25° C. (Bu₃Sn)₂ (8.0 g, 13.74 mmol) was added slowly and the reaction mixture was stirred at 100° C. under N₂ for 16 h. The mixture was allowed to cool to rt and then was poured into an aqueous solution of KF. The aqueous mixture was extracted with EtOAc (100 mL×2), the combined organic layers concentrated and the crude purified by silica gel column (PE/EtOAc=1/0 to 1/1) to afford 3-methyl-6-(tributylstannyl)pyrimidin-4(3H)-one (720 mg, 26.1% yield) as yellow oil. LCMS m/z=401.1 [M+H]⁺

### Preparation 364

### tert-butyl (2-acetamido-5-(3-(dimethylamino)prop-1-yn-1-yl)pyridin-4-yl)carbamate

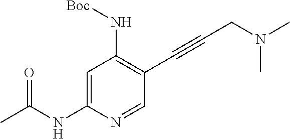 [SMILES: CC(=O)Nc1cc(NC(=O)OC(C)(C)C)c(C#CCN(C)C)cn1]

To a solution of tert-butyl (2-acetamido-5-bromopyridin-4-yl)carbamate (Preparation 53, 1.0 g, 3.03 mmol) and N,N-dimethylprop-2-yn-1-amine (377.7 mg, 4.54 mmol) in DMF (10 mL) was added TEA (459.7 mg, 4.54 mmol), CuI (57.7 mg, 0.303 mmol) and Pd(PPh₃)₂Cl₂ (106.3 mg, 0.151 mmol) and the reaction mixture was stirred at 90° C. for 12 h under N₂. The mixture was treated with H₂O (30 mL) and extracted with EtOAc (30 mL×3). The combined organic phase was washed with brine (30 mL×2), dried over Na₂SO₄ and filtered. The filtrate was concentrated under reduced pressure and the residue was purified by chromatography on silica gel (PE/EtOAc=20/1 to 0/1) to give tert-butyl (2-acetamido-5-(3-(dimethylamino)prop-1-yn-1-yl)pyridin-4-yl)carbamate (590.6 mg, 58.7% yield) as a white solid. ¹H NMR (500 MHz, CDCl₃) δ ppm: 8.93 (s, 1H), 8.18 (s, 1H), 8.13 (s, 1H), 7.32 (s, 1H), 3.57 (s, 2H), 2.38 (s, 6H), 2.19 (s, 3H), 1.54 (s, 9H).

### Preparation 365

### tert-butyl (2-acetamido-5-(3-methoxyprop-1-yn-1-yl)pyridin-4-yl)carbamate

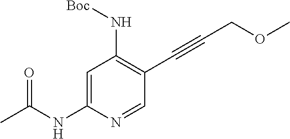 [SMILES: COCC#Cc1cnc(NC(C)=O)cc1NC(=O)OC(C)(C)C]

tert-Butyl (2-acetamido-5-(3-methoxyprop-1-yn-1-yl)pyridin-4-yl)carbamate was obtained as a yellow solid, 605 mg, 62.6% yield, from tert-butyl (2-acetamido-5-bromopyridin-4-yl)carbamate (Preparation 53) and 3-methoxyprop-1-yne, following the procedure described in Preparation 364. ¹H NMR (400 MHz, CDCl₃) δ ppm: 8.94 (s, 1H), 8.21 (s, 1H), 8.19 (s, 1H), 4.39 (s, 2H), 3.47 (s, 3H), 2.19 (s, 3H), 1.55 (s, 9H).

### Preparation 366

### tert-butyl (2-acetamido-5-(3-(dimethylamino)propyl)pyridin-4-yl)carbamate

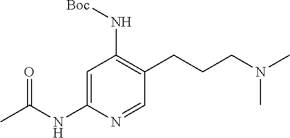 [SMILES: CC(=O)Nc1cc(NC(=O)OC(C)(C)C)c(CCCN(C)C)cn1]

To a solution of tert-butyl (2-acetamido-5-(3-(dimethylamino)prop-1-yn-1-yl)pyridin-4-yl)carbamate (Preparation 364, 570.6 mg, 1.72 mmol) in THF (8 mL) was added Pd/C (182.7 mg, 0.172 mmol, 10% purity). The resulting mixture was stirred at 20° C. for 2 h under H₂ (15 psi). The reaction was filtered, the filtrate concentrated under reduced pressure and the residue was purified by chromatography on silica gel (DCM/MeOH=20/1 to 10/1) to give tert-butyl (2-acetamido-5-(3-(dimethylamino)propyl)pyridin-4-yl)carbamate (547.2 mg, 94.8% yield) as a white solid. 1H NMR (400 MHz, CDCl₃) δ ppm: 10.88 (s, 1H), 8.70 (s, 1H), 8.35 (s, 1H), 7.92 (s, 1H), 2.57-2.60 (m, 2H), 2.24 (s, 6H), 2.13-2.16 (m, 3H), 2.10-2.12 (m, 2H), 1.76-1.80 (m, 2H), 1.52 (s, 9H).

### Preparation 367

### tert-butyl (2-acetamido-5-(3-methoxypropyl)pyridin-4-yl)carbamate

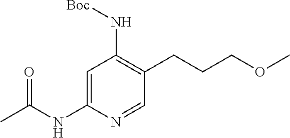 [SMILES: COCCCc1cnc(NC(C)=O)cc1NC(=O)OC(C)(C)C]

tert-Butyl (2-acetamido-5-(3-methoxypropyl)pyridin-4-yl)carbamate was obtained as a white solid, 532 mg, 90.4%, from tert-butyl (2-acetamido-5-(3-methoxyprop-1-yn-1-yl)pyridin-4-yl)carbamate (Preparation 365), following a similar procedure to that described in Preparation 366. ¹H NMR (400 MHz, CDCl₃) δ ppm: 8.86 (s, 1H), 8.08 (s, 1H), 7.97 (s, 1H), 7.91 (s, 1H), 3.41 (s, 3H), 3.29-3.32 (m, 2H), 2.61-2.65 (m, 2H), 2.18 (s, 3H), 1.80-1.87 (m, 2H), 1.54 (s, 9H).

### Preparation 368

### Tert-butyl (2-acetamido-5-cyclopropylpyridin-4-yl)carbamate

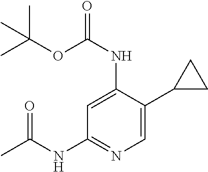 [SMILES: CC(=O)Nc1cc(NC(=O)OC(C)(C)C)c(C2CC2)cn1]

A vial containing tert-butyl N-(2-acetamido-5-bromo-4-pyridyl)carbamate (329 mg, 0.998 mmol), potassium cyclopropyltrifluoroborate (299 mg, 2.02 mmol), cataCXium A (74 mg, 0.205 mmol), KOAc (24 mg, 0.108 mmol) and Cs₂CO₃ (1.03 g, 3.16 mmol) in toluene (4.5 mL) and water (0.45 mL) was degassed then backfilled with N₂ then heated at 90° C. for 17 h. The cooled mixture was filtered through Celite®, washing through with EtOAc. The filtrate was concentrated in vacuo and the residue purified by silica gel column eluting with (20-85% EtOAc in heptane) to give tert-butyl (2-acetamido-5-cyclopropylpyridin-4-yl)carbamate (234 mg, 81% yield) as a white solid. LCMS m/z=292.1 [M+H]⁺.

### Preparation 369

### N-(4-amino-5-(3-(dimethylamino)propyl)pyridin-2-yl)acetamide

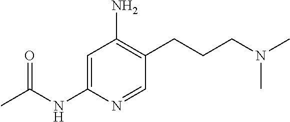 [SMILES: CC(=O)Nc1cc(N)c(CCCN(C)C)cn1]

To a solution of tert-butyl (2-acetamido-5-(3-(dimethylamino)propyl)pyridin-4-yl)carbamate (Preparation 366, 527.2 mg, 1.57 mmol) in DCM (5 mL) was added HCl/dioxane (4 M, 2 mL) and the reaction mixture was stirred at 20° C. for 3 h. The mixture was adjusted to pH 6 with NH₃·H₂O, the mixture was concentrated and was purified by Prep-HPLC (Method D, 0-20%) to give N-(4-amino-5-(3-(dimethylamino)propyl)pyridin-2-yl)acetamide (345.2 mg, 80.8% yield) as a white solid. ¹H NMR (400 MHz, DMSO-d₆) δ ppm: 13.00 (s, 1H), 11.77 (s, 1H), 10.65 (s, 1H), 7.75 (s, 1H), 6.65 (s, 1H), 3.03-3.08 (m, 2H), 2.72 (s, 6H), 2.51-2.52 (m, 2H), 2.17 (s, 3H), 1.79-1.87 (m, 2H).

### Preparation 370

### N-(4-amino-5-(3-methoxypropyl)pyridin-2-yl)acetamide hydrochloride

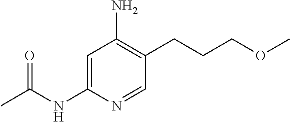 [SMILES: COCCCc1cnc(NC(C)=O)cc1N]

To a solution of tert-butyl (2-acetamido-5-(3-methoxypropyl)pyridin-4-yl)carbamate (Preparation 367, 521.7 mg, 1.61 mmol) in DCM (5 mL) was added HCl/dioxane (4 M, 2 mL). The resulting mixture was stirred at 20° C. for 3 h. The reaction was concentrated to give N-(4-amino-5-(3-methoxypropyl)pyridin-2-yl)acetamide hydrochloride (425.6 mg, crude) as a yellow solid. LCMS m/z=224.1 [M+H]⁺

### Preparation 371

### N-(4-amino-5-cyclopropylpyridin-2-yl)acetamide

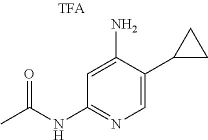

To a vial containing tert-butyl (2-acetamido-5-cyclopropylpyridin-4-yl)carbamate (Preparation 368, 234 mg, 0.804 mmol) in DCM (4 mL) was added TFA (0.6 mL, 7.8 mmol) and the reaction was stirred at 23° C. for 19 h. The mixture was diluted with DCM then neutralized by addition of 1.5 M aqueous K₂CO₃ solution. After 15 minutes of stirring at 23° C., the organic layer was separated, then dried over anhydrous MgSO₄. The mixture was filtered and the filtrate evaporated under reduced pressure to give N-(4-amino-5-cyclopropylpyridin-2-yl)acetamide (131 mg, crude). LCMS m/z=192.1 [M+H]⁺.

### Preparation 372

### 4,6-dichloro-N-methoxy-N-methylnicotinamide

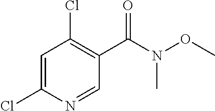

To a solution of 4,6-dichloronicotinic acid (25.0 g, 130 mmol) and N,O-dimethylhydroxylamine (13.9 g, 143.2 mmol) in THF (250 mL) was added TEA (19.5 g, 195.3 mmol) and EDCI (37.4 g, 195.3 mmol) and the reaction mixture was stirred at 25° C. for 12 h. The mixture was concentrated, diluted with H₂O (50 mL) and extracted with EtOAc (50 mL×3). The organic phase was washed with brine (30 mL), dried over Na₂SO₄, filtered and concentrated in vacuo. The residue was purified by column chromatography (PE/EtOAc=15/1 to 3/1) on silica gel to give 4,6-dichloro-N-methoxy-N-methylnicotinamide (5.4 g, 17.6% yield) as yellow oil. LCMS m/z=235.0 [M+H]

### Preparation 373

### cyclopropyl(4,6-dichloropyridin-3-yl)methanone

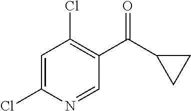 [SMILES: O=C(c1cnc(Cl)cc1Cl)C1CC1]

To a solution of 4,6-dichloro-N-methoxy-N-methylnicotinamide (Preparation 372, 1.0 g, 4.25 mmol) in THF (30 mL) was added dropwise cyclopropyl magnesium bromide (0.5 M, 51.1 mL) at 0° C. under N₂. The mixture was stirred at 25° C. for 1 h under N₂. The mixture was quenched at 0° C. with saturated ammonium chloride solution (100 mL) and extracted with EtOAc (50 mL×3). The organic phase was washed with brine (20 mL), dried over Na₂SO₄, filtered and concentrated in vacuo. The residue was purified by column chromatography (PE/EtOAc=15/1 to 5/1) on silica gel to give cyclopropyl(4,6-dichloropyridin-3-yl)methanone (590 mg, 64.2% yield) as yellow oil. LCMS m/z=216.1 [M+H]⁺

### Preparation 374

### 1-(4,6-dichloropyridin-3-yl)propan-1-one

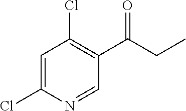

To a solution of 4,6-dichloronicotinic acid (2.0 g, 10.42 mmol) in THF (5 mL) was added ethyl magnesium bromide (1 M, 39.58 mL) at 0° C. The mixture was stirred at 25° C. for 2 h under N₂. The mixture was quenched with NH₄Cl (sat, 50 mL) and extracted with EtOAc (60 mL×3). The combined organic phase was washed with brine (60 mL), dried over anhydrous Na₂SO₄, filtered and concentrated. The crude was purified by column chromatography (PE/EtOAc=1/0 to 3/1) to give 1-(4,6-dichloropyridin-3-yl)propan-1-one (458.3 mg, 21.6% yield) as a light-yellow solid. LCMS m/z=204.0 [M+H]⁺

### Preparation 375

### 2,4-dichloro-5-(cyclopropyldifluoromethyl)pyridine

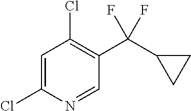 [SMILES: FC(F)(c1cnc(Cl)cc1Cl)C1CC1]

A solution of cyclopropyl(4,6-dichloropyridin-3-yl)methanone (Preparation 373, 2.8 g, 12.87 mmol) in DAST (20.0 mL) was stirred at 80° C. for 32 h. The mixture was diluted with H₂O (200 mL), extracted with DCM (50 mL×3), dried over Na₂SO₄, filtered and concentrated under reduced pressure. The crude was purified by column chromatography (PE/EtOAc=100/1 to 20/1) to give 2,4-dichloro-5-(cyclopropyldifluoromethyl)pyridine (900 mg, 29.4% yield) as brown oil. ¹H NMR (400 MHz, CDCl₃) δ ppm: 8.52 (s, 1H), 7.47 (s, 1H), 1.71-1.83 (m, 1H), 0.81-0.85 (m, 2H), 0.71-0.75 (m, 2H).

### Preparation 376

### 2,4-dichloro-5-(1,1-difluoropropyl)pyridine

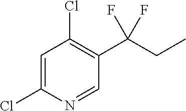

To a solution of 1-(4,6-dichloropyridin-3-yl)propan-1-one (Preparation 374, 458.0 mg, 2.24 mmol) in DCM (1 mL) was added DAST (3.7 g, 22.7 mmol) and the reaction mixture was stirred at 50° C. for 7 h. The mixture was diluted with water (10 mL) and extracted with DCM (20 mL×3). The combined organic phase was washed with brine (20 mL), dried over anhydrous Na₂SO₄, filtered and concentrated. The crude was purified by column chromatography (PE) to give 2,4-dichloro-5-(1,1-difluoropropyl)pyridine (352.2 mg, 69.4% yield) as light-yellow oil. ¹H NMR (500 MHz, CDCl₃) δ ppm: 8.54 (s, 1H), 7.45 (s, 1H), 2.27-2.37 (m, 2H), 1.01 (t, J=7.5 Hz, 3H).

### Preparation 377

### 2-chloro-5-(cyclopropyldifluoromethyl)-N-(4-methoxybenzyl)pyridin-4-amine

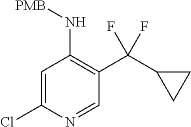

To a solution of 2,4-dichloro-5-(cyclopropyldifluoromethyl)pyridine (Preparation 375, 600 mg, 2.52 mmol) in DMSO (10 mL) was added PMBNH₂ (484.0 mg, 3.53 mmol) and TEA (510.1 mg, 5.04 mmol) and the reaction mixture was stirred at 70° C. for 16 h. The mixture was purified by prep-HPLC (Method P, Gradient 45-75%) to give 2-chloro-5-(cyclopropyldifluoromethyl)-N-(4-methoxybenzyl)pyridin-4-amine (232.8 mg, 27.3% yield) as a light-yellow solid. LCMS m/z=339.1 [M+H]⁺

### Preparation 378

### 2-chloro-5-(1,1-difluoropropyl)-N-(4-methoxybenzyl)pyridin-4-amine

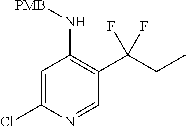

2-Chloro-5-(1,1-difluoropropyl)-N-(4-methoxybenzyl)pyridin-4-amine was obtained as a white solid, 111.3 mg, 38.5% from 2,4-dichloro-5-(1,1-difluoropropyl)pyridine (Preparation 376), following a similar procedure to that described in Preparation 377. LCMS m/z=327.1 [M+H]⁺

### Preparation 379

### N-(5-(cyclopropyldifluoromethyl)-4-((4-methoxybenzyl)amino)pyridin-2-yl)acetamide

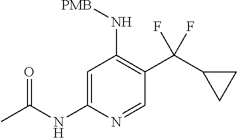

To a solution of 2-chloro-5-(cyclopropyldifluoromethyl)-N-(4-methoxybenzyl)pyridin-4-amine (Preparation 377, 50.0 mg, 0.148 mmol) in dioxane (5 mL) was added acetamide (43.6 mg, 0.738 mmol), BrettPhos Pd G3 (13.4 mg, 14.8 umol) and Cs₂CO₃ (120.2 mg, 0.369 mmol). The mixture was stirred at 90° C. for 30 min under N₂. The mixture was diluted with water (5 mL) and extracted with EtOAc (10 mL×3). The combined organic phase was washed with brine (10 mL), dried over anhydrous Na₂SO₄, filtered and concentrated. The crude was purified by column chromatography (PE/EtOAc=1/0 to 1/2) to give N-(5-(cyclopropyldifluoromethyl)-4-((4-methoxybenzyl)amino)pyridin-2-yl)acetamide (50.0 mg, crude) as a light-yellow solid. LCMS m/z=362.0 [M+H]⁺

### Preparation 380

### N-(5-(1,1-difluoropropyl)-4-((4-methoxybenzyl)amino)pyridin-2-yl)acetamide

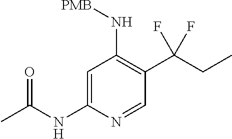

N-(5-(1,1-Difluoropropyl)-4-((4-methoxybenzyl)amino)pyridin-2-yl)acetamide was obtained, 120 mg, as a light yellow-solid, from 2-chloro-5-(1,1-difluoropropyl)-N-(4-methoxybenzyl)pyridin-4-amine (Preparation 378) and acetamide, following the procedure described in Preparation 379. LCMS m/z=350.1 [M+H]⁺

### Preparation 381

### N-(4-amino-5-(cyclopropyldifluoromethyl)pyridin-2-yl)acetamide

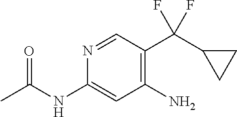 [SMILES: CC(=O)Nc1cc(N)c(C(F)(F)C2CC2)cn1]

To a solution of N-(5-(cyclopropyldifluoromethyl)-4-((4-methoxybenzyl)amino)pyridin-2-yl)acetamide (Preparation 379, 50.0 mg, 0.138 mmol) in MeCN (3 mL) and water (0.3 mL) was added ceric ammonium nitrate (227.6 mg, 0.415 mmol) and the reaction mixture stirred at 25° C. for 16 h. The mixture was purified by prep-HPLC (Method W, Gradient: 0-35%) to give N-(4-amino-5-(cyclopropyldifluoromethyl)pyridin-2-yl)acetamide (20.6 mg, 61.7% yield) as a white solid. LCMS m/z=242.1 [M+H]⁺

### Preparation 382

### N-(4-amino-5-(1,1-difluoropropyl)pyridin-2-yl)acetamide trifluoroacetate

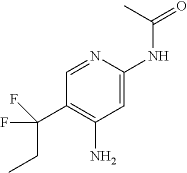 [SMILES: CCC(F)(F)c1cnc(NC(C)=O)cc1N]

To a solution of N-(5-(1,1-difluoropropyl)-4-((4-methoxybenzyl)amino)pyridin-2-yl)acetamide (Preparation 380, 60.0 mg, 0.172 mmol) in DCM (1 mL) was added TFA (3 mL) and the reaction mixture stirred at 25° C. for 16 h. The mixture was concentrated and purified by prep-TLC (PE/EtOAc=1/2) to give N-(4-amino-5-(1,1-difluoropropyl)pyridin-2-yl)acetamide trifluoroacetate (32.5 mg, crude) as a white solid. LCMS m/z=230.2 [M+H]⁺

### Preparation 383

### methyl 2-chloro-5-(dimethylamino)isonicotinate

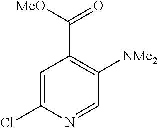 [SMILES: COC(=O)c1cc(Cl)ncc1N(C)C]

To a solution of methyl 2-chloro-5-fluoroisonicotinate (1 g, 5.28 mmol) in DMSO (8 mL) was added dimethylamine hydrochloride (473 mg, 5.80 mmol) and DIPEA (2.1 g, 15.83 mmol) and the mixture stirred at 25° C. for 16 h. The reaction was quenched with H₂O (20 mL) and extracted with EtOAc (20 mL×2). The combined organics were washed with brine (20 mL×2), dried (Na₂SO₄) and concentrated under reduced pressure. The residue was purified by chromatography on silica gel (33% EtOAc/PE) to afford methyl 2-chloro-5-(dimethylamino)isonicotinate as a yellow oil (960 mg, 84%). LCMS m/z=214.9 [M+H]⁺.

### Preparation 384 to 385

The compounds I the following table were prepared from methyl 2-chloro-5-fluoroisonicotinate and the appropriate amine using an analogous method to that described for Preparation 383.

| Preparation | Name, Structure, Starting materials (SM), Data |
|---|---|
| 384 | methyl 2-chloro-5-(pyrrolidin-1-yl)isonicotinate |
| | 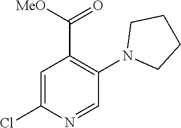 [SMILES: COC(=O)c1cc(Cl)ncc1N1CCCC1] |
| | SM: pyrrolidine |
| | LCMS m/z = 287.0 [M + H]⁺. |
| 385 | Methyl 2-chloro-5-morpholinoisonicotinate |
| | 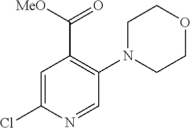 [SMILES: COC(=O)c1cc(Cl)ncc1N1CCOCC1] |
| | SM: morpholine |
| | ¹H NMR (500 MHz, CDCl₃) δ ppm: 8.16 (s, 1H), 7.56 (s, 1H), 3.93 (s, 3H), |
| | 3.84-3.85 (m, 4H), 3.09-3.10 (m, 4H). |

### Preparation 386

### 2-chloro-5-(dimethylamino)isonicotinic acid

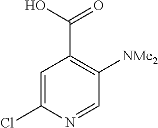 [SMILES: CN(C)c1cnc(Cl)cc1C(=O)O]

To a solution of methyl 2-chloro-5-(dimethylamino)isonicotinate (Preparation 383, 960 mg, 4.47 mmol) in H₂O (4 mL) and THF (12 mL) was added LiOH·H₂O (321 mg, 13.42 mmol) and the resulting mixture stirred at 25° C. for 16 h. The mixture was concentrated and purified by prep-HPLC (Boston Green ODS 150×30 mm, 5 mm; 18-38% MeCN/H₂O (TFA)) to give 2-chloro-5-(dimethylamino)isonicotinic acid as a white solid (300 mg, 28%). ¹H NMR (400 MHz, CDCl₃) δ ppm: 8.63 (s, 1H), 8.11 (s, 1H), 2.92 (s, 6H).

### Preparation 387 to 388

The title compounds were prepared from the corresponding ester using an analogous method to that described for Preparation 386.

| Preparation | Name, Structure, SM, Data |
|---|---|
| 387 | 2-chloro-5-(pyrrolidin-1-yl)isonicotinic acid |
| | 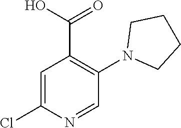 [SMILES: O=C(O)c1cc(Cl)ncc1N1CCCC1] |
| | SM: methyl 2-chloro-5-(pyrrolidin-1-yl)isonicotinate (Preparation 384) |
| | LCMS m/z = 271.0 [M + H]⁺. |
| 388 | 2-chloro-5-morpholinoisonicotinic acid |
| | 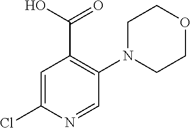 [SMILES: O=C(O)c1cc(Cl)ncc1N1CCOCC1] |
| | SM: methyl 2-chloro-5-morpholinoisonicotinate (Preparation 385) |
| | ¹H NMR (400 MHz, MeOH-d₄) δ ppm: 8.34 (s, 1H), 7.69 (s, 1H), 3.78-3.75 |
| | (m, 4H), 3.15-3.18 (m, 4H). |

### Preparation 389

### 2-bromo-5-(4-methyl-1H-pyrazol-1-yl)isonicotinic acid

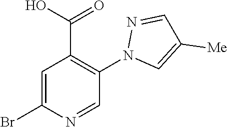 [SMILES: Cc1cnn(-c2cnc(Br)cc2C(=O)O)c1]

To a solution of methyl 2-bromo-5-fluoroisonicotinate (1.0 g, 4.27 mmol) in DMF (10 mL) was added 4-methyl-H-pyrazole (1.0 g, 4.27 mmol) and Cs₂CO₃ (4.2 g, 12.82 mmol) and the reaction stirred at 80° C. for 16 h. The mixture was diluted with H₂O (200 mL) and the pH adjusted to pH ˜2 with aqueous 1000 HCL. The mixture was extracted with EtOAc (100 mL×3), dried (Na₂SO₄) and concentrated under reduced pressure. The residue was purified by prep-HPLC (Method Y, Gradient 12-42%) to give 2-bromo-5-(4-methyl-1H-pyrazol-1-yl)isonicotinic acid as a white solid (220 mg, 18%). LCMS m/z=282.0 [M+H]⁺.

### Preparation 390 to 393

The title compounds were prepared from methyl 2-bromo-5-fluoroisonicotinate and the appropriate heterocycle using an analogous method to that described for Preparation 389.

| Preparation | Name/Structure/SM/Data |
|---|---|
| 390 | 2-bromo-5-(4-methoxy-1H-pyrazol-1-yl)isonicotinic acid |
| | 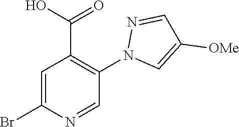 [SMILES: COc1cnn(-c2cnc(Br)cc2C(=O)O)c1] |
| | Het: 4-methoxy-1H-pyrazole |
| | LCMS m/z = 300.0 [M + H]⁺. |
| 391 | 2-bromo-5-(4-cyano-1H-pyrazol-1-yl)isonicotinic acid |
| | 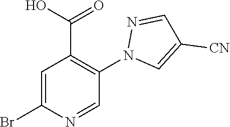 [SMILES: N#Cc1cnn(-c2cnc(Br)cc2C(=O)O)c1] |
| | Het: 1H-pyrazole-4-carbonitrile |
| | LCMS m/z = 294.9 [M + H]⁺. |
| 392 | 2-bromo-5-(3-methyl-1H-1,2,4-triazol-1-yl)isonicotinic acid |
| | 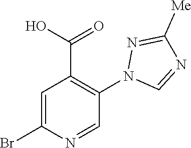 [SMILES: Cc1ncn(-c2cnc(Br)cc2C(=O)O)n1] |
| | Het: 3-methyl-1H-1,2,4-triazole |
| | ¹H NMR (400 MHz, DMSO-d₆) δ: ppm 8.90 (s, 1H), 8.77 (s, 1H), 8.05 (s, |
| | 1H), 2.34 (s, 3H) |
| 393 | 2-bromo-5-(4-(2-hydroxyethyl)-1H-pyrazol-1-yl)isonicotinic acid |
| | 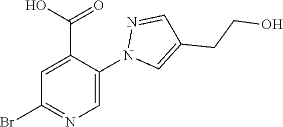 [SMILES: O=C(O)c1cc(Br)ncc1-n1cc(CCO)cn1] |
| | Het: 2-(1H-pyrazol-4-yl)ethan-1-ol |
| | LCMS m/z = 312.1 [M + H]⁺. |

### Preparation 394

### 2-bromo-5-(4-(2-methoxyethyl)-1H-pyrazol-1-yl)isonicotinic acid

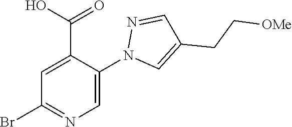 [SMILES: COCCc1cnn(-c2cnc(Br)cc2C(=O)O)c1]

2-Bromo-5-(4-(2-methoxyethyl)-1H-pyrazol-1-yl)isonicotinic acid was obtained as a white solid, 800 mg, 96%, from 2-bromo-5-(4-(2-hydroxyethyl)-1H-pyrazol-1-yl)isonicotinic acid (Preparation 393) and MeI, following a similar procedure to that described in Preparation 293. LCMS m/z=326.1 [M+H]⁺.

### Preparation 395

### tert-butyl (2-chloro-5-(dimethylamino)pyridin-4-yl)carbamate

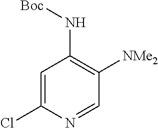 [SMILES: CN(C)c1cnc(Cl)cc1NC(=O)OC(C)(C)C]

Et₃N (756 mg, 7.48 mmol) and DPPA (2.1 g, 7.48 mmol) were added to a solution of 2-chloro-5-(dimethylamino)isonicotinic acid (Preparation 386, 750 mg, 3.74 mmol) in t-BuOH (10 mL) and stirred at 90° C. for 3 h under N₂. The reaction was quenched with H₂O (20 mL) and extracted with EtOAc (20 mL×2). The combined organics were washed with brine (20 mL×2), dried (Na₂SO₄) and evaporated to dryness. The residue was purified by chromatography on silica gel (100% EtOAc) to give tert-butyl (2-chloro-5-(dimethylamino)pyridin-4-yl)carbamate as a yellow oil (1 g, 98%). ¹H NMR (400 MHz, CDCl₃) δ ppm: 8.09 (s, 1H), 8.08 (s, 1H), 2.69 (s, 6H), 1.55 (s, 9H).

### Preparation 396

### tert-butyl (2-acetamido-5-(dimethylamino)pyridin-4-yl)carbamate

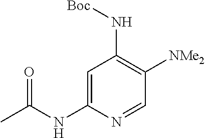 [SMILES: CC(=O)Nc1cc(NC(=O)OC(C)(C)C)c(N(C)C)cn1]

To a solution of tert-butyl (2-chloro-5-(dimethylamino)pyridin-4-yl)carbamate (Preparation 395, 1 g, 3.68 mmol) in dioxane (10 mL) was added acetamide (1.5 g, 25.8 mmol), Cs₂CO₃ (2.4 g, 7.36 mmol), Xantphos (852 mg, 1.47 mmol) and Pd₂(dba)₃ (337 mg, 0.37 mmol) and the reaction stirred at 100° C. for 16 h under N₂. The reaction mixture was concentrated and the residue purified by prep-HPLC (Method T, Gradient 18-38%) to afford tert-butyl (2-acetamido-5-(dimethylamino)pyridin-4-yl)carbamate as a yellow solid (300 mg, 28%). LCMS m/z=295.2 [M+H]⁺.

### Preparation 397

### N-(4-amino-5-(dimethylamino)pyridin-2-yl)acetamide hydrochloride

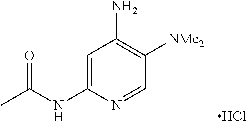

To a solution of tert-butyl (2-acetamido-5-(dimethylamino)pyridin-4-yl)carbamate (Preparation 396, 120 mg, 0.41 mmol) in DCM (2 mL) was added HCl/dioxane (4 M, 0.4 mL) and the mixture stirred at 25° C. for 3 h. The reaction was concentrated to give N-(4-amino-5-(dimethylamino)pyridin-2-yl)acetamide hydrochloride as a yellow solid (110 mg). LCMS m/z=195.1 [M+H]⁺.

### Preparation 398 to 405

The following compounds were prepared from the appropriate carboxylic acid, following a similar 3 step synthesis to that described for the synthesis of Preparation 397

| Preparation | Name, Structure, SM, Data |
|---|---|
| 398 | N-(4-amino-5-(pyrrolidin-1-yl)pyridin-2-yl)acetamide hydrochloride |
| | 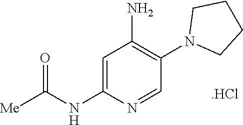 [SMILES: CC(=O)Nc1cc(N)c(N2CCCC2)cn1 ; ".HCl"] |
| | SM:2-chloro-5-(pyrrolidin-1-yl)isonicotinic acid (Preparation 387) |
| | LCMS m/z = 221.2 [M + H]⁺. |
| 399 | N-(4-amino-5-morpholinopyridin-2-yl)acetamide hydrochloride |
| | 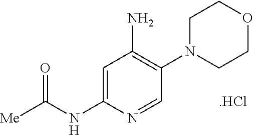 [SMILES: CC(=O)Nc1cc(N)c(N2CCOCC2)cn1 ; ".HCl"] |
| | SM:2-chloro-5-morpholinoisonicotinic acid (Preparation 388) |
| | LCMS m/z = 237.0 [M + H]⁺. |
| 400 | N-(4-amino-5-(4-methyl-1H-pyrazol-1-yl)pyridin-2-yl)acetamide |
| | hydrochloride |
| | 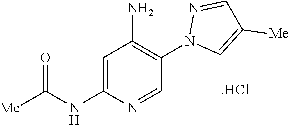 [SMILES: CC(=O)Nc1cc(N)c(-n2cc(C)cn2)cn1 ; ".HCl"] |
| | SM: 2-bromo-5-(4-methyl-1H-pyrazol-1-yl)isonicotinic acid (Preparation |
| | 389) |
| | LCMS m/z = 232.2 [M + H]⁺. |
| 401 | N-(4-amino-5-(4-methoxy-1H-pyrazol-1-yl)pyridin-2-yl)acetamide |
| | hydrochloride |
| | 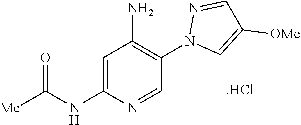 [SMILES: COc1cnn(-c2cnc(NC(C)=O)cc2N)c1 ; ".HCl"] |
| | SM: 2-bromo-5-(4-methoxy-1H-pyrazol-1-yl)isonicotinic acid (Preparation |
| | 390) |
| 402 | N-(4-amino-5-(4-cyano-1H-pyrazol-1-yl)pyridin-2-yl)acetamide |
| | hydrochloride |
| | 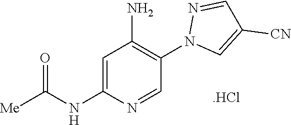 [SMILES: CC(=O)Nc1cc(N)c(-n2cc(C#N)cn2)cn1 ; ".HCl"] |
| | SM: 2-bromo-5-(4-cyano-1H-pyrazol-1-yl)isonicotinic acid (Preparation |
| | 391) |
| | ¹H NMR (400 MHz, MeOH-d₄) δ ppm: 8.69 (s, 1H), 8.24 (s, 1H), 8.15 (s, |
| | 1H), 6.61 (s, 1H), 2.27 (s, 3H). |
| 403 | N-(4-amino-5-(3-methyl-1H-1,2,4-triazol-1-yl)pyridin-2-yl)acetamide |
| | hydrochloride |
| | 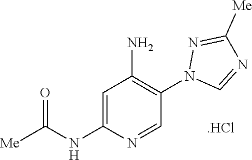 [SMILES: CC(=O)Nc1cc(N)c(-n2cnc(C)n2)cn1 ; ".HCl"] |
| | SM: 2-bromo-5-(3-methyl-1H-1,2,4-triazol-1-yl)isonicotinic acid |
| | (Preparation 392) |
| | LCMS m/z = 233.1 [M + H]⁺. |
| 404 | N-(4-amino-5-(4-(2-methoxyethyl)-1H-pyrazol-1-yl)pyridin-2-yl)acetamide |
| | hydrochloride |
| | 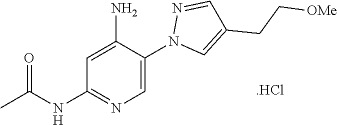 [SMILES: COCCc1cnn(-c2cnc(NC(C)=O)cc2N)c1 ; ".HCl"] |
| | SM: 2-bromo-5-(4-(2-methoxyethyl)-1H-pyrazol-1-yl)isonicotinic acid |
| | (Preparation 394) |
| | LCMS m/z = 276.0 [M + H]⁺. |
| 405 | N-(4-amino-5-(4-(methoxymethyl)-1H-pyrazol-1-yl)pyridin-2-yl)acetamide |
| | hydrochloride |
| | 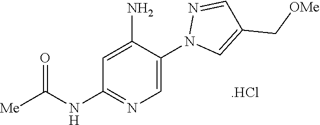 [SMILES: COCc1cnn(-c2cnc(NC(C)=O)cc2N)c1 ; ".HCl"] |
| | SM: 2-bromo-5-(4-(2-methoxymethyl)-1H-pyrazol-1-yl)isonicotinic acid |
| | LCMS m/z = 262.1 [M + H]⁺. |

### Preparation 406

### tert-butyl (6′-acetamido-5-cyano-[2,3′-bipyridin]-4′-yl)carbamate

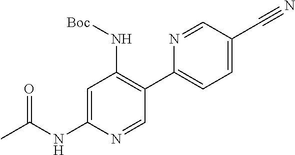 [SMILES: CC(=O)Nc1cc(NC(=O)OC(C)(C)C)c(-c2ccc(C#N)cn2)cn1]

To a solution of tert-butyl (2-acetamido-5-bromopyridin-4-yl)carbamate (Preparation 53, 1.0 g, 3.03 mmol) in MeOH (25 mL), toluene (25 mL) and water (10 mL) was added 6-bromonicotinonitrile (554.3 mg, 3.03 mmol), (BPin)₂ (1.5 g, 6.06 mmol), Pd(OAc)₂ (136.0 mg, 0.606 mmol), CsF (2.8 g, 18.17 mmol) and bis(1-adamantyl)-butyl-phosphane (434.4 mg, 1.21 mmol) and the reaction mixture was stirred at 100° C. for 2 h under N₂. The mixture was diluted with water (15 mL) and extracted with EtOAc (20 mL×3). The combined organic phase was washed with brine (60 mL), dried over anhydrous Na₂SO₄, filtered and concentrated. The crude was purified by column chromatography (PE/EtOAc=0/1 to 1/2) to give tert-butyl (6′-acetamido-5-cyano-[2,3′-bipyridin]-4′-yl)carbamate (147.0 mg, 137% yield) as a yellow solid. ¹H NMR (500=z, CDCl₃) δ ppm: 11.39 (s, 1H), 9.26 (s, 1H), 8.94 (d, J=2.0 Hz, 1H), 8.54 (s, 1H), 8.33 (s, 1H), 8.07-8.10 (m, 1H), 7.87 (d, J=8.5 Hz, 1H), 2.25 (s, 3H), 1.55 (s, 9H).

### Preparation 407 to 419

The compounds in the following table were prepared from tert-butyl (2-acetamido-5-bromopyridin-4-yl)carbamate (Preparation 53) and the appropriate halo heterocycle, following a similar procedure to that described in Preparation 406.

| Preparation | |
|---|---|
| No | Name, Structure, Starting material (SM), Data |
| 407 | tert-butyl (6′-acetamido-6-(difluoromethoxy)-[2,3′-bipyridin]-4′-yl)carbamate |
| | 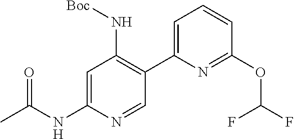 [SMILES: CC(=O)Nc1cc(NC(=O)OC(C)(C)C)c(-c2cccc(OC(F)F)n2)cn1] |
| | SM: 2-chloro-6-(difluoromethoxy)pyridine. |
| | 110 mg, 50.1% as a white solid. LCMS m/z = 395.2 [M + H]⁺ |
| 408 | tert-butyl (6′-acetamido-5-fluoro-[2,3'-bipyridin]-4′-yl)carbamate |
| | 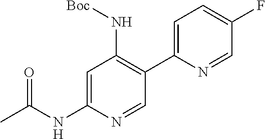 [SMILES: CC(=O)Nc1cc(NC(=O)OC(C)(C)C)c(-c2ccc(F)cn2)cn1] |
| | SM: 2-bromo-5-fluoropyridine |
| | 120 mg, 38.1% as yellow solid. ¹H NMR (400 MHz, DMSO-d₆) δ ppm 11.35 |
| | (s, 1H), 10.51 (s, 1H), 8.95 (s, 1H), 8.67-8.72 (m, 2H), 8.10-8.15 (m, 1H), |
| | 7.94-7.96 (m, 1H), 2.10 (s, 3H), 1.48 (s, 9H). |
| 409 | tert-butyl (2-acetamido-5-(4-methyl-5-oxo-4,5-dihydropyrazin-2-yl)pyridin- |
| | 4-yl)carbamate |
| | 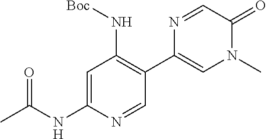 [SMILES: CC(=O)Nc1cc(NC(=O)OC(C)(C)C)c(-c2cn(C)c(=O)cn2)cn1] |
| | SM: 5-bromo-1-methylpyrazin-2(1H)-one |
| | 446 mg, 41.0% as a brown solid. ¹H NMR (500MHz, CDCl₃) δ ppm: 9.52 (s, |
| | 1H), 9.03 (br s, 1H), 8.27 (br s, 1H), 8.22 (s, 1H), 8.09 (s, 1H), 7.52 (s, 1H), |
| | 3.65 (s, 3H), 2.20 (s, 3H), 1.50 (s, 9H). |
| 410 | tert-butyl (2-acetamido-5-(2-methoxypyrimidin-4-yl)pyridin-4-yl)carbamate |
| | 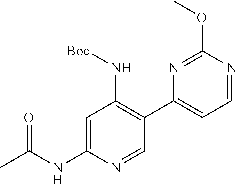 [SMILES: COc1nccc(-c2cnc(NC(C)=O)cc2NC(=O)OC(C)(C)C)n1] |
| | SM: 4-chloro-2-methoxypyrimidine |
| | prep-HPLC (Method X, Gradient 25-55%). 275.0 mg, 25.3% yield as a white |
| | solid. LCMS m/z = 360.1 [M + H]⁺ |
| 412 | tert-butyl (2-acetamido-5-(2-methylpyrimidin-4-yl)pyridin-4-yl)carbamate |
| | 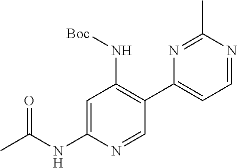 [SMILES: CC(=O)Nc1cc(NC(=O)OC(C)(C)C)c(-c2ccnc(C)n2)cn1] |
| | SM: 4-chloro-2-methylpyrimidine |
| | 220 mg, 42.3% yield as a yellow solid. ¹H NMR (500 MHz, CDCl₃) δ: ppm |
| | 12.37 (s, 1H), 9.22 (s, 1H), 8.70 (d, J = 5.5 Hz, 1H), 8.62 (s, 1H), 8.21 (s, 1H), |
| | 7.54 (d, J = 5.5 Hz, 1H), 2.79 (s, 3H), 2.24 (s, 3H), 1.56 (s, 9H). |
| 413 | tert-butyl (2-acetamido-5-(5-fluoropyrimidin-2-yl)pyridin-4-yl)carbamate |
| | 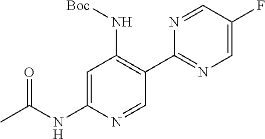 [SMILES: CC(=O)Nc1cc(NC(=O)OC(C)(C)C)c(-c2ncc(F)cn2)cn1] |
| | SM: 2-bromo-5-fluoropyrimidine |
| | 550 mg, 65.4% as yellow solid. ¹H NMR (500 MHz, CDCl₃) δ: ppm 11.57 |
| | (s, 1H), 9.30 (s, 2H), 8.70 (s, 2H), 8.52 (s, 1H), 2.23 (s, 3H), 1.57 (s, 9H). |
| 414 | tert-butyl (2-acetamido-5-(6-methoxypyrimidin-4-yl)pyridin-4-yl)carbamate |
| | 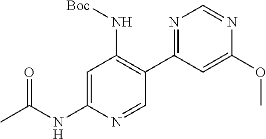 [SMILES: COc1cc(-c2cnc(NC(C)=O)cc2NC(=O)OC(C)(C)C)ncn1] |
| | SM: 4-chloro-6-methoxypyrimidine |
| | 300 mg, 55.1% as a white solid. LCMS m/z = 360.1 [M + H]⁺ |
| 415 | tert-butyl (2-acetamido-5-(6-methylpyrimidin-4-yl)pyridin-4-yl)carbamate |
| | 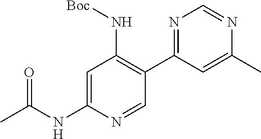 [SMILES: CC(=O)Nc1cc(NC(=O)OC(C)(C)C)c(-c2cc(C)ncn2)cn1] |
| | SM: 4-chloro-6-methylpyrimidine |
| | 156 mg, as a yellow solid. LCMS m/z = 344.3 [M + H]⁺ |
| 416 | tert-butyl (2-acetamido-5-(6-methoxy-2-methylpyrimidin-4-yl)pyridin-4- |
| | yl)carbamate |
| | 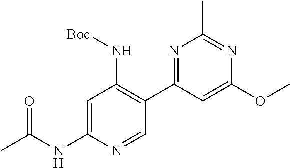 [SMILES: COc1cc(-c2cnc(NC(C)=O)cc2NC(=O)OC(C)(C)C)nc(C)n1] |
| | SM: 4-chloro-6-methoxy-2-methylpyrimidine |
| | 280 mg, 49.6% yield as a white solid. ¹H NMR (400 MHz, CDCl₃) δ ppm: |
| | 12.24 (s, 1H), 9.14 (br s, 1H), 8.51 (s, 1H), 8.18 (s, 1H), 6.88 (s, 1H), 4.01 (s, |
| | 3H), 2.67 (s, 3H), 2.22 (s, 3H), 1.54 (s, 9H). |
| 417 | tert-butyl (2-acetamido-5-(2-methoxy-6-methylpyrimidin-4-yl)pyridin-4- |
| | yl)carbamate |
| | 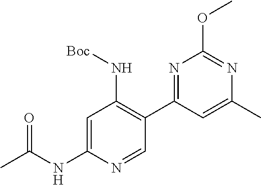 [SMILES: COc1nc(C)cc(-c2cnc(NC(C)=O)cc2NC(=O)OC(C)(C)C)n1] |
| | SM: 4-chloro-2-methoxy-6-methylpyrimidine |
| | 350 mg, 31.0% as a yellow solid. ¹H NMR (400 MHz, CDCl₃) δ: ppm 9.23 |
| | (br s, 1H), 8.59 (s, 1H), 8.41 (br s, 1H), 7.21 (s, 1H), 4.09 (s, 3H), 2.54 (s, |
| | 3H), 2.23 (s, 3H), 1.52 (s, 9H). |
| 418 | tert-butyl (2-acetamido-5-(5-methoxypyridazin-3-yl)pyridin-4-yl)carbamate |
| | 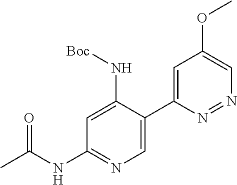 [SMILES: COc1cnnc(-c2cnc(NC(C)=O)cc2NC(=O)OC(C)(C)C)c1] |
| | SM: 3-chloro-5-methoxypyridazine |
| | LCMS m/z = 360.1 [M + H]⁺ |
| 419 | tert-butyl (2-acetamido-5-(1-methyl-1H-1,2,4-triazol-3-yl)pyridin-4- |
| | yl)carbamate |
| | 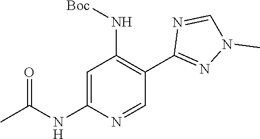 [SMILES: CC(=O)Nc1cc(NC(=O)OC(C)(C)C)c(-c2ncn(C)n2)cn1] |
| | SM: 3-bromo-1-methyl-1H-1,2,4-triazole |
| | 350 mg, 69.5% as yellow solid. 1H NMR (400 MHz, CDCl₃) δ ppm 9.11 (br |
| | s, 1H), 8.04 (br s, 1H), 7.93 (d, J = 4.4 Hz, 1H), 6.30 (s, 1H), 3.94 (s, 3H), 1.57 |
| | (s, 3H), 1.49 (s, 9H). |

### Preparation 420

### (6-acetamido-4-((tert-butoxycarbonyl)amino)pyridin-3-yl)boronic acid

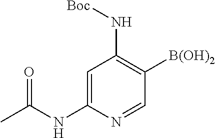 [SMILES: CC(=O)Nc1cc(NC(=O)OC(C)(C)C)c(B(O)O)cn1]

To a solution of tert-butyl (2-acetamido-5-bromopyridin-4-yl)carbamate (Preparation 53, 500 mg, 1.51 mmol) in dioxane (5 mL) was added (BPin)₂ (769.1 mg, 3.03 mmol), KOAc (297.2 mg, 3.03 mmol) and Pd(dppf)Cl₂ (110.8 mg, 0.151 mmol). The resulting mixture was stirred at 90° C. for 1 h under N₂. The mixture was concentrated under reduced pressure to give (6-acetamido-4-((tert-butoxycarbonyl)amino)pyridin-3-yl)boronic acid (600 mg, crude) as a brown solid. LCMS m/z=296.1 [M+H]⁺

### Preparation 421

### tert-butyl (2-acetamido-5-(4,4,5,5-tetramethyl-1,3,2-dioxaborolan-2-yl)pyridin-4-yl)carbamate

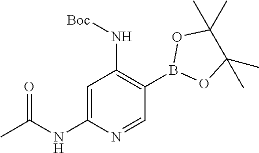 [SMILES: CC(=O)Nc1cc(NC(=O)OC(C)(C)C)c(B2OC(C)(C)C(C)(C)O2)cn1]

To a solution of tert-butyl (2-acetamido-5-bromopyridin-4-yl)carbamate (Preparation 53, 2 g, 6.1 mmol) in dioxane (60 mL) was added KOAc (1.2 g, 12.1 mmol), XPhos Pd G3 (512.7 mg, 0.606 mmol) and (BPin)₂ (1.5 g, 6.1 mmol). The resulting mixture was stirred at 90° C. for 16 h under N₂. The mixture was concentrated to give tert-butyl (2-acetamido-5-(4,4,5,5-tetramethyl-1,3,2-dioxaborolan-2-yl)pyridin-4-yl)carbamate (6 g, crude), which was used directly in the next step.

### Preparation 422

### tert-butyl (2-acetamido-5-(1-methyl-6-oxo-1,6-dihydropyridazin-3-yl)pyridin-4-yl)carbamate

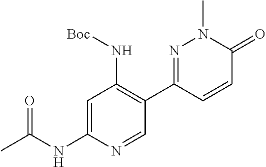 [SMILES: CC(=O)Nc1cc(NC(=O)OC(C)(C)C)c(-c2ccc(=O)n(C)n2)cn1]

To a solution of tert-butyl (2-acetamido-5-(4,4,5,5-tetramethyl-1,3,2-dioxaborolan-2-yl)pyridin-4-yl)carbamate (Preparation 421, 3.7 g, 2.0 mmol) in dioxane (40 mL) and water (8 mL) was added 6-chloro-2-methylpyridazin-3(2H)-one (340.3 mg, 2.4 mmol), Pd(dppf)Cl₂·DCM (160.2 mg, 0.196 mmol) and K₂CO₃ (542.2 mg, 3.9 mmol) and the mixture was stirred at 100° C. for 2 h under N₂. The reaction mixture was concentrated under reduced pressure to give residue, which was partitioned between H₂O (15 mL) and EtOAc (15 mL) and the layers separated. The aqueous was extracted with EtOAc (15 mL×3), dried over Na₂SO₄, filtered and concentrated. The residue was purified with silica gel chromatography (ISCO®, EtOAc) to give tert-butyl (2-acetamido-5-(1-methyl-6-oxo-1,6-dihydropyridazin-3-yl)pyridin-4-yl)carbamate (335 mg, 39.9% yield) as a yellow solid. LCMS m/z=360.0 [M+H]⁺

### Preparation 423 to 428

The compounds in the following table were prepared from tert-butyl (2-acetamido-5-(4,4,5,5-tetramethyl-1,3,2-dioxaborolan-2-yl)pyridin-4-yl)carbamate (Preparation 421) or (6-acetamido-4-((tert-butoxycarbonyl)amino)pyridin-3-yl)boronic acid (Preparation 420) and the appropriate halo heterocycle, following a similar procedure to that described in Preparation 422.

| Preparation | |
|---|---|
| No | Name, Structure, Starting Material (SM), Data |
| 423 | 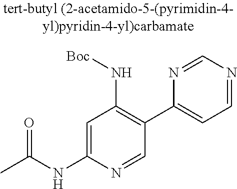 [SMILES: CC(=O)Nc1cc(NC(=O)OC(C)(C)C)c(-c2ccncn2)cn1] |
| | SM: Preparation 420 and 4-bromo |
| | pyrimidine hydrobromideyellow solid, |
| | 300 mg. LCMS m/z = 330.1 [M + H]⁺ |
| 424 | 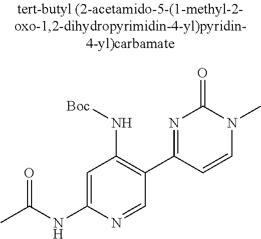 [SMILES: CC(=O)Nc1cc(NC(=O)OC(C)(C)C)c(-c2ccn(C)c(=O)n2)cn1] |
| | SM: Preparation 421 and 4-chloro-1- |
| | methylpyrimidin-2(1H)-one (150 mg, |
| | 39.4% yield) as a yellow solid. ¹H NMR |
| | (400 MHz, MeOD-d₄) δ ppm: 9.10 (s, |
| | 1H), 8.75 (s, 1H), 8.16 (d, J = 6.8 Hz, |
| | 1H), 7.07 (d, J = 6.8 Hz, 1H), 3.60 (s, |
| | 3H), 2.20 (s, 3H), 1.55 (s, 9H). |
| 425 | 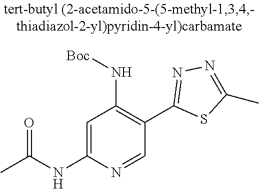 [SMILES: CC(=O)Nc1cc(NC(=O)OC(C)(C)C)c(-c2nnc(C)s2)cn1] |
| | SM: Preparation 421 and 2-bromo-5- |
| | methyl-1,3,4-thiadiazole(150 mg, 54.0% |
| | yield) as a yellow solid. ¹H NMR (400 |
| | MHz, CDCl₃) δ: ppm 11.04 (s, 1H), 9.31 |
| | (s, 1H), 8.40 (s, 1H), 8.09 (s, 1H), 2.84 |
| | (s, 3H), 2.23 (s, 3H), 1.55 (s, 9H). |
| 426 | 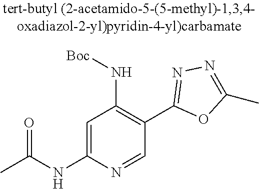 [SMILES: CC(=O)Nc1cc(NC(=O)OC(C)(C)C)c(-c2nnc(C)o2)cn1] |
| | SM: Preparation 421 and 2-bromo-5- |
| | methyl-1,3,4-oxadiazole (100 mg, |
| | 28.3% yield) as a yellow solid. ¹H NMR |
| | (400 MHz, CDCl₃) δ: ppm 10.32 (s, 1H), |
| | 9.34 (s, 1H), 8.65 (s, 1H), 8.39 (s, 1H), |
| | 2.66 (s, 3H), 2.25 (s, 3H), 1.56 (s, 9H). |
| 427 | 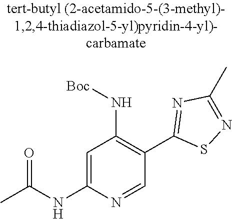 [SMILES: CC(=O)Nc1cc(NC(=O)OC(C)(C)C)c(-c2nc(C)ns2)cn1] |
| | SM: Preparation 421 and 5-bromo-3- |
| | methyl-1,2,4-thiadiazole (88.5 mg, |
| | 47.8% yield) as a white solid. ¹H |
| | NMR (500 MHz, CDCl₃) δ ppm: |
| | 11.21 (s, 1H), 9.24 (s, 1H), 8.54 (s, |
| | 1H), 8.02 (s, 1H), 2.74 (s, 3H), 2.23 |
| | (s, 3H), 1.57 (s, 9H). |
| 428 | 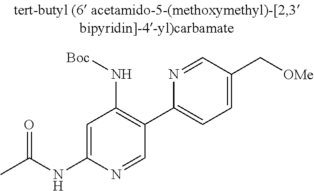 [SMILES: COCc1ccc(-c2cnc(NC(C)=O)cc2NC(=O)OC(C)(C)C)nc1] |
| | SM: Preparation 420 and 2-bromo-5-methoxy- |
| | methylpyridine (4.4 g, 55.7% yield) as a yellow |
| | solid. ¹H NMR: (400 MHz, DMSO-d₆) δ ppm: |
| | 12.04 (s, 1H), 10.52 (s, 1H), 9.00 (s, 1H), 8.75 |
| | (s, 1H), 8.62 (d, J = 1.6 Hz, 1H), 8.09 (d, J = |
| | 8.4 Hz, 1H), 7.92-7.94 (m, 1H), 4.52 (s, 2H), |
| | 3.54 (s, 3H), 2.11 (s, 3H), 1.50 (s, 9H). |

### Preparation 429

### tert-butyl (2-acetamido-5-(6-methoxypyridazin-3-yl)pyridin-4-yl)carbamate and tert-butyl (2-acetamido-5-(6-oxo-1,6-dihydropyridazin-3-yl)pyridin-4-yl)carbamate

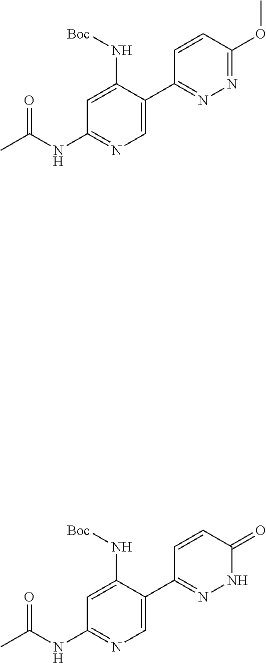

To a solution of (6-acetamido-4-((tert-butoxycarbonyl)amino)pyridin-3-yl)boronic acid (Preparation 420, 300 mg, 1.02 mmol) in dioxane (5 mL) and H₂O (1 mL) was added 3-bromo-6-methoxypyridazine (288 mg, 1.52 mmol), Pd(dppf)Cl₂-DCM (83 mg, 0.10 mmol), K₂CO₃ (281 mg, 2.03 mmol) and the mixture was stirred at 90° C. for 2 h under N₂. The mixture was diluted with water (30 mL) and extracted with EtOAc (20 mL×3). The combined organic layers were washed with brine (30 mL), dried over Na₂SO₄, filtered and concentrated and the residue purified by chromatography on silica gel (5-66% EtOAc/PE) to give a mixture of tert-butyl (2-acetamido-5-(6-methoxypyridazin-3-yl)pyridin-4-yl)carbamate and tert-butyl (2-acetamido-5-(6-oxo-1,6-dihydropyridazin-3-yl)pyridin-4-yl)carbamate as a yellow solid (180 mg, 49%) which was used in the next step. ¹H NMR (400 MHz, MeOH-d₄) δ ppm: 9.07 (s, 1H), 8.58 (s, 1H), 8.12 (d, J=9.6 Hz, 1H), 7.31 (d, J=9.2 Hz, 1H), 4.16 (s, 3H), 2.20 (s, 3H), 1.55 (s, 9H).

### Preparation 430

### Tert-butyl (2-acetamido-5-(6-methylpyridazin-3-yl)pyridin-4-yl)carbamate

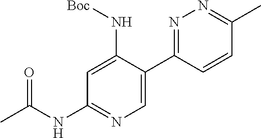 [SMILES: CC(=O)Nc1cc(NC(=O)OC(C)(C)C)c(-c2ccc(C)nn2)cn1]

A vial containing tert-butyl (2-acetamido-5-(4,4,5,5-tetramethyl-1,3,2-dioxaborolan-2-yl)pyridin-4-yl)carbamate (Preparation 421, 378 mg, 1.0 mmol), 3-bromo-6-methyl-pyridazine (173 mg, 1.0 mmol), Pd(dppf)Cl₂: DCM (12 mg, 15 umol) and K₃PO₄ (646 mg, 3.0 mmol) in dioxane (4 mL) and water (0.4 mL) was degassed, then backfilled with N₂ and heated to 90° C. for 22 h. The mixture was cooled to rt then filtered through a Celite® plug, washing through with EtOAc. The filtrate was concentrated and the residue purified by column chromatography on silica gel eluting with (15-100% EtOAc in heptane) to give tert-butyl (2-acetamido-5-(6-methylpyridazin-3-yl)pyridin-4-yl)carbamate (160 mg, 46% yield) as a white solid. LCMS m/z=343.9 [M+H]⁺.

### Preparation 431

### tert-butyl (2-acetamido-5-(1-methyl-6-oxo-1,6-dihydropyrimidin-4-yl)pyridin-4-yl)carbamate

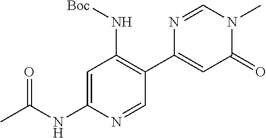 [SMILES: CC(=O)Nc1cc(NC(=O)OC(C)(C)C)c(-c2cc(=O)n(C)cn2)cn1]

To a solution of 3-methyl-6-(tributylstannyl)pyrimidin-4(3H)-one (Preparation 363, 500 mg, 1.51 mmol) and tert-butyl (2-acetamido-5-bromopyridin-4-yl)carbamate (Preparation 53, 604.5 mg, 1.51 mmol) in DMF (5 mL) was added PdCl₂(PPh₃)₂ (53.2 mg, 0.076 mmol) and the reaction mixture was stirred at 100° C. for 4 h under N₂. The mixture was quenched with aq. KF (20 mL), diluted with H₂O (10 mL) and extracted with DCM (15 mL×3). The combined organic phase was washed with brine (20 mL), dried with Na₂SO₄, filtered and concentrated in vacuo. The crude was purified by column chromatography (DCM/MeOH=1/0 to 10/1) on silica gel to give tert-butyl (2-acetamido-5-(1-methyl-6-oxo-1,6-dihydropyrimidin-4-yl)pyridin-4-yl)carbamate (220 mg, 40.4% yield) as yellow solid. LCMS m/z=360.1 [M+H]⁺

### Preparation 432

### Tert-butyl (2-acetamido-5-(5-methyl-4,5,6,7-tetrahydropyrazolo[1,5-a]pyrazin-2-yl)pyridin-4-yl)carbamate

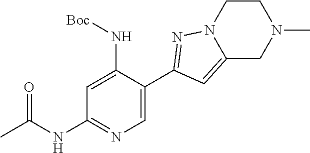 [SMILES: CC(=O)Nc1cc(NC(=O)OC(C)(C)C)c(-c2cc3n(n2)CCN(C)C3)cn1]

A vial containing 2-bromo-5-methyl-4,5,6,7-tetrahydropyrazolo[1,5-a]pyrazine (Preparation 331, 439 mg, 2.03 mmol), tert-butyl (2-acetamido-5-(4,4,5,5-tetramethyl-1,3,2-dioxaborolan-2-yl)pyridin-4-yl)carbamate (Preparation 421, 1.15 g, 3.05 mmol), SPhos-Pd-G3 (157.4 mg, 0.202 mmol), and K₃PO₄ (1.5 M aqueous solution, 4.1 mL, 6.15 mmol) in 2-MeTHF (8 mL) was degassed then backfilled with N₂ then heated to 75° C. After 3 h, the mixture was cooled to rt then filtered through Celite®, rinsing through with EtOAc, and the filtrate was washed with brine. The organic layer was dried over anhydrous Na₂SO₄ then filtered and concentrated under reduced pressure. The residue was purified by silica gel column chromatography (20-100% 3:1 EtOAc: EtOH in heptane) to afford tert-butyl (2-acetamido-5-(5-methyl-4,5,6,7-tetrahydropyrazolo[1,5-a]pyrazin-2-yl)pyridin-4-yl)carbamate (160 mg, 20% yield) as a white solid. LCMS m/z=387.2 [M+H]⁺.

### Preparation 433

### 2-chloro-5-(6-methoxypyridazin-3-yl)pyridin-4-amine

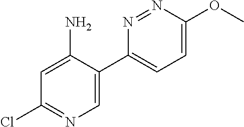 [SMILES: COc1ccc(-c2cnc(Cl)cc2N)nn1]

A mixture of 2-chloropyridin-4-amine (1 g, 7.78 mmol), 2-MeTHF (10 mL) and HBPin (1.20 g, 9.38 mmol) was heated at 90° C. for 2 h in a MW reactor. The reaction was cooled to rt, vented, (BPin)₂ (1 g, 3.94 mmol), (1Z,5Z)-cycloocta-1,5-diene; 2,4-dimethyl-BLAHbicyclo[1.1.0]butane (50 mg, 75.43 mmol), and 3,4,7,8-tetramethyl-1,10-phenanthroline (50 mg, 211.59 mmol) were added and the reaction was sealed and heated to 90° C. for 90 mins. The cooled reaction mixture was diluted with water (5 mL), the mixture stirred for 15 mins and sparged with N₂. K₃PO₄ (3.30 g, 15.56 mmol), Pd(OAc)₂ (45 mg, 0.20 mmol), PCy₃ (110 mg, 0.392 mmol) and 3-chloro-6-methoxy-pyridazine (2 g, 13.84 mmol) were added, the reaction then sealed and heated to 100° C. for 16 h. The cooled reaction mixture was diluted with water (5 mL) and EtOAc (5 mL), and the biphasic mixture was filtered through Celite®, rinsing with 10 mL EtOAc. The crude solid was suspended in MeOH (30-40 mL), and passed through Celite®, leaving residual black solids. The filtrate was concentrated to dryness and the residue re-crystallised from hot IPA to give 2-chloro-5-(6-methoxypyridazin-3-yl)pyridin-4-amine (1.25 g, 67.9% yield). LCMS m/z=236.9 [M+H]⁺.

### Preparation 434

### N-(4-amino-5-(6-methoxypyridazin-3-yl)pyridin-2-yl)acetamide

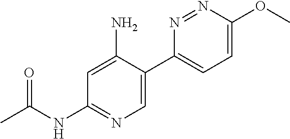 [SMILES: COc1ccc(-c2cnc(NC(C)=O)cc2N)nn1]

A mixture of 2-chloro-5-(6-methoxypyridazin-3-yl)pyridin-4-amine (Preparation 433, 1.25 g, 5.28 mmol), acetamide (1.60 g, 27.04 mmol), Cs₂CO₃ (5.32 g, 16.34 mmol) and BrettPhos Pd G3 (100 mg, 0.110 mmol) in 2-MeTHF (5 mL) was stirred at 95° C. overnight. Additional BrettPhos Pd G3 (100 mg, 0.110 mmol) was added and the reaction stirred for a further 3 h. The cooled reaction mixture was diluted with water (10 mL) and stirred vigorously. The mixture was filtered through filter paper, rinsing with EtOAc (10 mL) and dried under vacuum to give N-(4-amino-5-(6-methoxypyridazin-3-yl)pyridin-2-yl)acetamide (550 mg, 40.16% yield). LCMS m/z=260.0 [M+H]⁺.

### Preparation 435

### tert-butyl (tert-butoxycarbonyl)(2-chloro-5-cyanopyridin-4-yl)carbamate

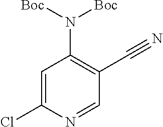 [SMILES: CC(C)(C)OC(=O)N(C(=O)OC(C)(C)C)c1cc(Cl)ncc1C#N]

To a solution of 4-amino-6-chloronicotinonitrile (600 mg, 3.91 mmol) in DCM (10 mL) was added TEA (1.09 mL, 7.81 mmol), Boc₂O (3.4 g, 15.63 mmol) and DMAP (47.7 mg, 0.391 mmol) and the reaction mixture was stirred at 25° C. for 16 h. The mixture was concentrated and the residue was purified by chromatography on silica gel (PE/EtOAc=3/1) to give tert-butyl (tert-butoxycarbonyl)(2-chloro-5-cyanopyridin-4-yl)carbamate (1.1 g, 76.7% yield) as a yellow solid. ¹H NMR (500 MHz, DMSO-d₆) δ: ppm 9.06 (s, 1H), 8.14 (s, 1H), 1.40 (s, 18H).

### Preparation 436

### tert-butyl (2-acetamido-5-cyanopyridin-4-yl)(tert-butoxycarbonyl)carbamate

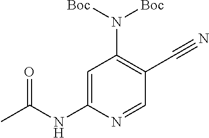 [SMILES: CC(=O)Nc1cc(N(C(=O)OC(C)(C)C)C(=O)OC(C)(C)C)c(C#N)cn1]

To a solution tert-butyl (tert-butoxycarbonyl)(2-chloro-5-cyanopyridin-4-yl)carbamate (Preparation 435, 1.1 g, 2.97 mmol) in dioxane (10 mL) was added acetamide (876.5 mg, 14.84 mmol), Pd₂(dba)₃ (271.8 mg, 0.297 mmol), Xantphos (343.4 mg, 0.594 mmol) and K₂CO₃ (820.3 mg, 5.94 mmol) and the reaction mixture was stirred at 120° C. for 4 h under N₂. The mixture was concentrated and the residue was purified by chromatography on silica gel (PE/EtOAc=1/1) to give tert-butyl (2-acetamido-5-cyanopyridin-4-yl)(tert-butoxycarbonyl)carbamate (800 mg, 71.6% yield) as a yellow solid. ¹H NMR (400 MHz, DMSO-d₆) δ: ppm 11.2 (s, 1H), 8.87 (s, 1H), 8.12 (s, 1H), 2.15 (s, 3H), 1.40 (s, 18H).

### Preparation 437

### tert-butyl (2-acetamido-5-(N-hydroxycarbamimidoyl)pyridin-4-yl)carbamate

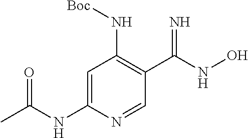 [SMILES: CC(=O)Nc1cc(NC(=O)OC(C)(C)C)c(C(=N)NO)cn1]

To a solution of tert-butyl (2-acetamido-5-cyanopyridin-4-yl)(tert-butoxycarbonyl)carbamate (Preparation 436, 790 mg, 2.10 mmol) in EtOH (12 mL) was added NH₂OH·HCl (291.7 mg, 4.20 mmol) and TEA (585 uL, 4.20 mmol) and the reaction was stirred at 90° C. for 16 h. The mixture was concentrated and the residue was purified by chromatography on silica gel (PE/EtOAc=1/1) to give tert-butyl (2-acetamido-5-(N-hydroxycarbamimidoyl)pyridin-4-yl)carbamate (400 mg, 61.6% yield) as a yellow solid. 1H NMR (400 MHz, DMSO-d₆) δ: ppm 10.91 (s, 1H), 10.45 (s, 1H), 10.06 (s, 1H), 8.93 (s, 1H), 8.45 (s, 1H), 6.22 (s, 2H), 2.08 (s, 3H), 1.49 (s, 9H).

### Preparation 438

### tert-butyl (2-acetamido-5-(5-methyl-1,2,4-oxadiazol-3-yl)pyridin-4-yl)carbamate

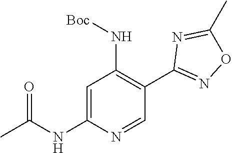 [SMILES: CC(=O)Nc1cc(NC(=O)OC(C)(C)C)c(-c2noc(C)n2)cn1]

To a solution of tert-butyl (2-acetamido-5-(N-hydroxycarbamimidoyl)pyridin-4-yl)carbamate (Preparation 437, 350 mg, 1.13 mmol) in dioxane (10 mL) was added 1,1-dimethoxy-N,N-dimethylethan-1-amine (753.5 mg, 5.66 mmol) and the reaction was stirred at 80° C. for 16 h. The mixture was concentrated and the residue was purified by chromatography on silica gel (EtOAc) to give tert-butyl (2-acetamido-5-(5-methyl-1,2,4-oxadiazol-3-yl)pyridin-4-yl)carbamate (300 mg, 79.5% yield) as a yellow solid. 1H NMR (500 MHz, DMSO-d₆) δ: ppm 10.69 (s, 1H), 9.55 (s, 1H), 9.04 (s, 1H), 8.83 (s, 1H), 2.70 (s, 3H), 2.11 (s, 3H), 1.51 (s, 9H).

### Preparation 439

### tert-butyl (2-amino-5-bromopyridin-4-yl)carbamate

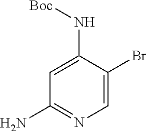 [SMILES: CC(C)(C)OC(=O)Nc1cc(N)ncc1Br]

To a flask containing tert-butyl (2-(acetamido-2,2,2-d3)pyridin-4-yl)carbamate (1.2 g, 4.7 mmol) in MeCN (12 mL) was added NBS (873 mg, 4.91 mmol) in portions at 23° C. After 5 mins, the mixture was heated at 70° C. for 1 h. The cooled reaction mixture was concentrated under reduced pressure, the residue was diluted with EtOAc then washed once with saturated aqueous NaHCO₃. The organic layer was dried over anhydrous Na₂SO₄ then filtered and concentrated under reduced pressure. The residue was purified by silica gel column (5-50% EtOAc in heptane) to give tert-butyl (2-amino-5-bromopyridin-4-yl)carbamate (729.2 mg, crude). LCMS m/z=287.9 [M+H]⁺.

### Preparation 440

### Tert-butyl (2-(acetamido-2,2,2-d3)-5-bromopyridin-4-yl)carbamate

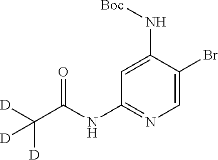 [SMILES: [2H]C([2H])([2H])C(=O)Nc1cc(NC(=O)OC(C)(C)C)c(Br)cn1]

To a vial containing tert-butyl (2-amino-5-bromopyridin-4-yl)carbamate (Preparation 439, 729 mg, 2.53 mmol) in anhydrous TH (8 mL) was added DIPEA (1.4 mL, 8.04 mmol) dropwise at <−15° C. After 5 mins, 2,2,2-trideuterioacetyl chloride (209 mg, 2.6 mmol) was added dropwise to the cold homogeneous solution. Upon complete addition, the reaction was warmed to 23° C. and stirred for 18 h. The reaction mixture was concentrated under reduced pressure, the residue was dissolved in EtOAc and rinsed with aqueous saturated NaHCO₃. The organic layer was dried over anhydrous Na₂SO₄, filtered and concentrated in vacuo. The residue was purified by silica gel column chromatography with (5-60% EtOAc in heptane) to afford a white solid as tert-butyl (2-(acetamido-2,2,2-d3)-5-bromopyridin-4-yl)carbamate (206 mg, 25% yield). LCMS m/z=332.9 [M+H]⁺.

### Preparation 441

### Tert-butyl (2-(acetamido-2,2,2-d3)-5-(4,4,5,5-tetramethyl-1,3,2-dioxaborolan-2-yl)pyridin-4-yl)carbamate

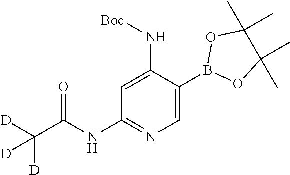 [SMILES: [2H]C([2H])([2H])C(=O)Nc1cc(NC(=O)OC(C)(C)C)c(B2OC(C)(C)C(C)(C)O2)cn1]

Tert-butyl (2-(acetamido-2,2,2-d3)-5-(4,4,5,5-tetramethyl-1,3,2-dioxaborolan-2-yl)pyridin-4-yl)carbamate was obtained from tert-butyl (2-(acetamido-2,2,2-d3)-5-bromopyridin-4-yl)carbamate

(Preparation 440), following the method described in Preparation 421.

### Preparation 442

### Tert-butyl (2-(acetamido-2,2,2-d3)-5-(6-methoxypyridazin-3-yl)pyridin-4-yl)carbamate

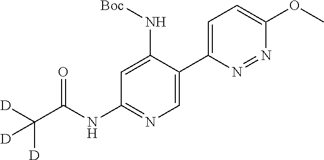 [SMILES: [2H]C([2H])([2H])C(=O)Nc1cc(NC(=O)OC(C)(C)C)c(-c2ccc(OC)nn2)cn1]

A vial containing tert-butyl (2-(acetamido-2,2,2-d3)-5-(4,4,5,5-tetramethyl-1,3,2-dioxaborolan-2-yl)pyridin-4-yl)carbamate (Preparation 441, 312 mg, 0.820 mmol), 3-chloro-6-methoxy-pyridazine (78 mg, 0.542 mmol), KOAc (13 mg, 60 umol), PCy₃ (36 mg, 0.129 mmol), and K₂CO₃ (1.5 M, 1.1 mL) in dioxane (4 mL) was degassed then backfilled with N₂ then heated to 95° C. and stirred for 2 h. The mixture was cooled to rt then filtered through a Celite® plug, rinsing through with EtOAc. The combined organic filtrate was concentrated in vacuo, and the residue was loaded onto a silica gel column and purified with (25-95% EtOAc in heptane) to afford an off white solid as tert-butyl (2-(acetamido-2,2,2-d3)-5-(6-methoxypyridazin-3-yl)pyridin-4-yl)carbamate (124 mg, 64% yield) LCMS m/z=363.1 [M+H]⁺.

### Preparation 443

### tert-butyl (6′-acetamido-5-(1-fluoroethyl)-[2,3′-bipyridin]-4′-yl)carbamate

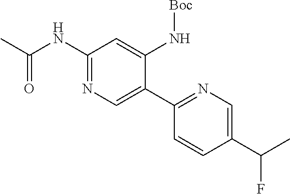 [SMILES: CC(=O)Nc1cc(NC(=O)OC(C)(C)C)c(-c2ccc(C(C)F)cn2)cn1]

A mixture of tert-butyl (2-acetamido-5-(4,4,5,5-tetramethyl-1,3,2-dioxaborolan-2-yl)pyridin-4-yl)carbamate (Preparation 421, 500 mg, 0.663 mmol), 2-bromo-5-(1-fluoroethyl)pyridine (Preparation 183, 159 mg, 0.994 mmol), Pd(PCy₃)₂ (44 mg, 66 mol) and aq. K₂CO₃ (2 M, 1.99 mmol, 994 μL) in dioxane (5 mL) was heated at 100° C. for 2 h. The cooled mixture was filtered and the filtrate was purified directly on silica gel (0-70% EtOAc-EtOH 3:1 with 2% NH₄OH in heptane) to give tert-butyl (6′-acetamido-5-(1-fluoroethyl)-[2,3′-bipyridin]-4′-yl)carbamate (105 mg, 42% yield). LCMS m/z=375 [M+H]⁺

### Preparation 444

### N-(4′-amino-5-(1-fluoroethyl)-[2,3′-bipyridin]-6′-yl)acetamide hydrochloride

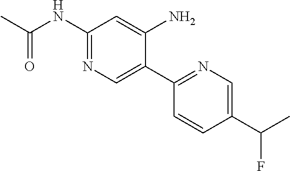 [SMILES: CC(=O)Nc1cc(N)c(-c2ccc(C(C)F)cn2)cn1]

To a solution of tert-butyl (6′-acetamido-5-(1-fluoroethyl)-[2,3′-bipyridin]-4′-yl)carbamate (Preparation 443, 100 mg, 267 μmol) in DCM (2 ml) was added HCl (4 M, 1.34 mmol, 334 μL) and the reaction stirred at rt for 1 h. The mixture was evaporated under reduced pressure to give N-(4′-amino-5-(1-fluoroethyl)-[2,3′-bipyridin]-6′-yl)acetamide hydrochloride (crude). LCMS m/z=275[M+H]⁺

### Preparations 445 to 449

The compounds in the following table were prepared from tert-butyl (2-acetamido-5-(4,4,5,5-tetramethyl-1,3,2-dioxaborolan-2-yl)pyridin-4-yl)carbamate (Preparation 421) and appropriate halopyridine, following a similar 2 step synthesis to that described for Preparation 444.

| Preparation | |
|---|---|
| No | Name, Structure, Starting materials (SM), Data |
| 445 | 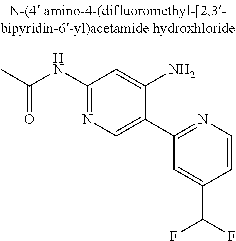 [SMILES: CC(=O)Nc1cc(N)c(-c2cc(C(F)F)ccn2)cn1] |
| | SM: 2-bromo-4-(difluoromethyl)pyridine |
| | LCMS m/z = 279 [M + H]⁺ |
| 446 | 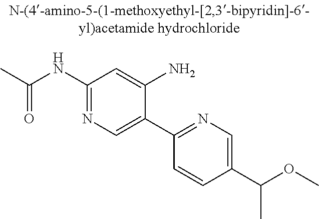 [SMILES: COC(C)c1ccc(-c2cnc(NC(C)=O)cc2N)nc1] |
| | SM: 2-bromo-5-(1-methoxyethyl)pyridine |
| | (Preparation 184). LCMS m/z = 287 [M + H]⁺ |
| 447 | 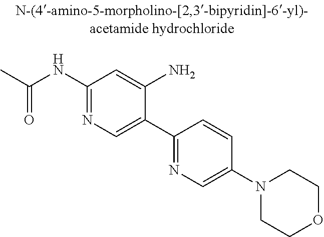 [SMILES: CC(=O)Nc1cc(N)c(-c2ccc(N3CCOCC3)cn2)cn1] |
| | SM: 4-(6-bromopyridin-3-yl)morpholine |
| | LCMS m/z = 314 [M + H]⁺ |
| 448 | 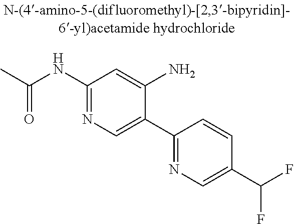 [SMILES: CC(=O)Nc1cc(N)c(-c2ccc(C(F)F)cn2)cn1] |
| | SM: 2-bromo-5-(difluoromethyl)pyridine |
| | LCMS m/z = 279 [M + H]⁺ |
| 449 | 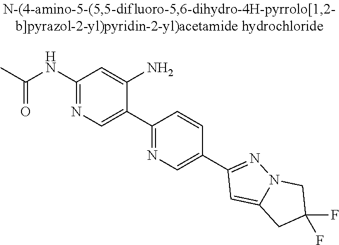 [SMILES: CC(=O)Nc1cc(N)c(-c2ccc(-c3cc4n(n3)CC(F)(F)C4)cn2)cn1] |
| | SM: 2-bromo-5,5-difluoro-5,6-dihydro-4H-pyrrolo |
| | [1,2-b] pyrazole LCMS m/z = 294 [M + H]⁺ |

### Preparation 450

### N-(4-amino-5-(6-methoxypyridazin-3-yl)pyridin-2-yl)acetamide hydrochloride and N-(4-amino-5-(6-oxo-1,6-dihydropyridazin-3-yl)pyridin-2-yl)acetamide hydrochloride

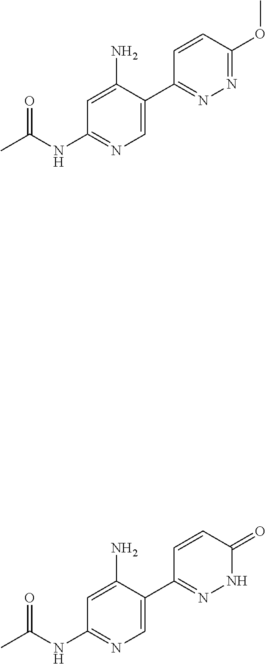

To a solution of a mixture of tert-butyl (2-acetamido-5-(6-methoxypyridazin-3-yl)pyridin-4-yl)carbamate and tert-butyl (2-acetamido-5-(6-oxo-1,6-dihydropyridazin-3-yl)pyridin-4-yl)carbamate (Preparation 429, 180 mg, 0.50 mmol) in DCM (1 mL) was added HCl/dioxane (4 M, 0.2 mL) and the resulting mixture was stirred at 25° C. for 2 h. The reaction mixture was concentrated under reduced pressure to give a mixture of N-(4-amino-5-(6-methoxypyridazin-3-yl)pyridin-2-yl)acetamide and N-(4-amino-5-(6-oxo-1,6-dihydropyridazin-3-yl)pyridin-2-yl)acetamide as a white solid (150 mg, HCl salt)

### Preparation 451

### N-(4′-amino-5-cyano-[2,3′-bipyridin]-6′-yl)acetamide hydrochloride

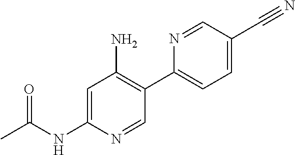 [SMILES: CC(=O)Nc1cc(N)c(-c2ccc(C#N)cn2)cn1]

To a solution of tert-butyl (6′-acetamido-5-cyano-[2,3′-bipyridin]-4′-yl)carbamate (Preparation 406, 147.0 mg, 0.416 mmol) in DCM (1.0 mL) was added HCl/EtOAc (4 M, 2.0 mL) and the reaction mixture was stirred at 25° C. for 1 h. The mixture was evaporated under reduced pressure to give N-(4′-amino-5-cyano-[2,3′-bipyridin]-6′-yl)acetamide hydrochloride (130.0 mg, crude) as a yellow solid. LCMS m/z=254.2 [M+H]⁺

### Preparation 452 to 470

The compounds in the following table were prepared from the appropriate protected amine, following the procedure described in Preparation 451.

| Preparation | |
|---|---|
| no | Name, Structure, Starting Material (SM), Data |
| 452 | 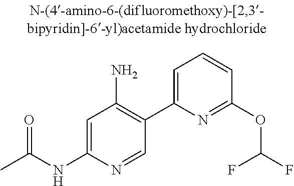 [SMILES: CC(=O)Nc1cc(N)c(-c2cccc(OC(F)F)n2)cn1] |
| | SM: tert-butyl (6′-acetamido-6-(difluorometh- |
| | oxy)-[2,3′-bipyridin]-4′-yl)carbamate (Prepar- |
| | ation 407) 160 mg, white solid. LCMS m/z = |
| | 295.1 [M + H]⁺ |
| 453 | 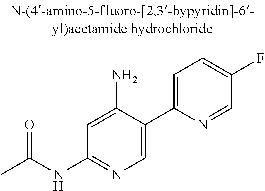 [SMILES: CC(=O)Nc1cc(N)c(-c2ccc(F)cn2)cn1] |
| | SM: tert-butyl (6'-acetamido-5-fluoro-[2,3'- |
| | bipyridin]-4'-yl)carbamate (Preparation 408) |
| | LCMS m/z = 247.1 [M + H]⁺ |
| 454 | 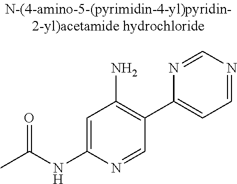 [SMILES: CC(=O)Nc1cc(N)c(-c2ccncn2)cn1] |
| | SM: tert-butyl (2-acetamido-5-(pyrimi |
| | din-4-yl)pyridin-4-yl)carbamate (Prep- |
| | aration 423)150 mg, white solid. LCMS |
| | m/z = 230.1 [M + H]⁺ |
| 455 | 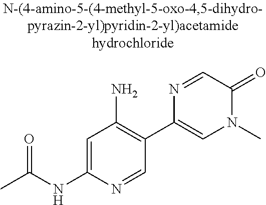 [SMILES: CC(=O)Nc1cc(N)c(-c2cn(C)c(=O)cn2)cn1] |
| | SM: tert-butyl (2-acetamido-5-(4-methyl-5- |
| | oxo-4,5-dihydropyrazin-2-yl)pyridin-4-yl)- |
| | carbamate (Preparation 409)203 mg, as |
| | yellow solid. |
| 456 | 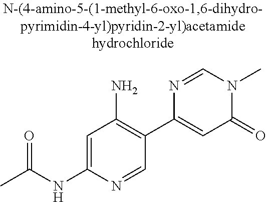 [SMILES: CC(=O)Nc1cc(N)c(-c2cc(=O)n(C)cn2)cn1] |
| | SM: tert-butyl (2-acetamido-5-(1-methyl- |
| | 6-oxo-1,6-dihydropyrimidin-4-yl)pyridin- |
| | 4-yl)carbamate (Preparation 431) 140 mg, |
| | as white solid. LCMS m/z = 260.1 [M + H]⁺ |
| 457 | 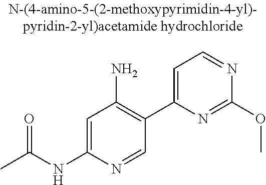 [SMILES: COc1nccc(-c2cnc(NC(C)=O)cc2N)n1] |
| | SM: tert-butyl (2-acetamido-5-(2-methoxy- |
| | pyrimidin-4-yl)pyridin-4-yl)carbamate |
| | (Preparation 410) 250.0 mg, as a white solid. |
| | LCMS m/z = 260.1 [M + H]⁺ |
| 458 | 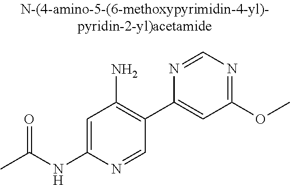 [SMILES: COc1cc(-c2cnc(NC(C)=O)cc2N)ncn1] |
| | SM: tert-butyl (2-acetamido-5-(6-methoxy- |
| | pyrimidin-4-yl)pyridin-4-yl)carbamate |
| | (Preparation 414) |
| 459 | 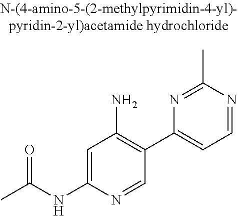 [SMILES: CC(=O)Nc1cc(N)c(-c2ccnc(C)n2)cn1] |
| | SM: tert-butyl (2-acetamido-5-(2- |
| | methylpyrimidin-4-yl)pyridin-4-yl)- |
| | carbamate (Preparation 412) 120 mg |
| | as a yellow solid. LCMS m/z = 244.1 |
| | [M + H]⁺ |
| 460 | 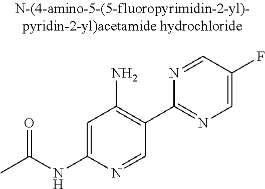 [SMILES: CC(=O)Nc1cc(N)c(-c2ncc(F)cn2)cn1] |
| | SM: tert-butyl (2-acetamido-5-(5-fluoro |
| | pyrimidin-2-yl)pyridin-4-yl)carbamate |
| | (Preparation 413) 320 mg as a yellow solid. |
| | LCMS m/z = 248.2 [M + H]⁺ |
| 461 | 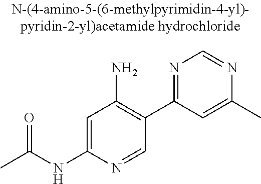 [SMILES: CC(=O)Nc1cc(N)c(-c2cc(C)ncn2)cn1] |
| | SM: tert-butyl (2-acetamido-5-(6-methyl- |
| | pyrimidin-4-yl)pyridin-4-yl)carbamate |
| | (Preparation 415) 60 mg, as a light-yellow |
| | solid. LCMS m/z = 244.1 [M + H]⁺ |
| 462 | 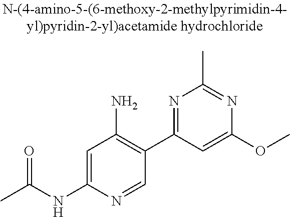 [SMILES: COc1cc(-c2cnc(NC(C)=O)cc2N)nc(C)n1] |
| | SM: tert-butyl (2-acetamido-5-(6-methoxy-2- |
| | methylpyrimidin-4-yl)pyridin-4-yl)carbamate |
| | (Preparation 416) 200 mg, as a white solid. |
| | LCMS m/z = 274.1 [M + H]⁺ |
| 463 | 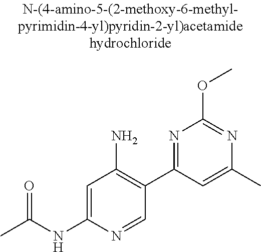 [SMILES: COc1nc(C)cc(-c2cnc(NC(C)=O)cc2N)n1] |
| | SM: tert-butyl (2-acetamido-5-(2-meth- |
| | oxy-6-methylpyrimidin-4-yl)pyridin-4- |
| | yl)carbamate (Preparation 417) 240 mg, |
| | as yellow solid. ¹H NMR (400 MHz, |
| | MeOD-d₄) δ: ppm 8.45 (s, 1H), 7.46 (s, |
| | 1H), 6.52 (s, 1H), 4.07 (s, 3H), 2.56 (s, |
| | 3H), 2.25 (s, 3H). |
| 464 | 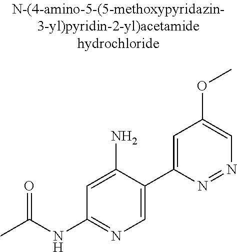 [SMILES: COc1cnnc(-c2cnc(NC(C)=O)cc2N)c1] |
| | SM: tert-butyl (2-acetamido-5-(5- |
| | methoxypyridazin-3-yl)pyridin-4-yl) |
| | carbamate (Preparation 418) LCMS |
| | m/z = 260.2 [M + H]⁺ |
| 465 | 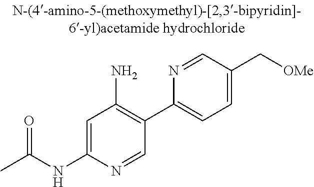 [SMILES: COCc1ccc(-c2cnc(NC(C)=O)cc2N)nc1] |
| | SM: tert-butyl (6′-acetamido-5-(methoxymethyl)- |
| | [2,3′-bipyridin]-4′-yl)carbamate (Preparation 428) |
| | 17.2 g, as yellow solid. LCMS m/z = 273.2 |
| | [M + H]⁺ |
| 466 | 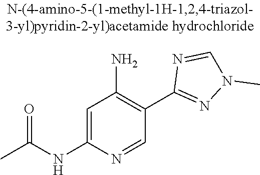 [SMILES: CC(=O)Nc1cc(N)c(-c2ncn(C)n2)cn1] |
| | SM: tert-butyl (2-acetamido-5-(1-methyl- |
| | 1H-1,2,4-triazol-3-yl)pyridin-4-yl)- |
| | carbamate (Preparation 419) 210 mg as |
| | yellow solid. LCMS m/z = 232.9 [M + H]⁺ |
| 467 | 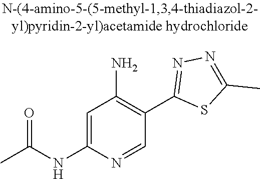 [SMILES: CC(=O)Nc1cc(N)c(-c2nnc(C)s2)cn1] |
| | SM: tert-butyl (2-acetamido-5-(5-methyl- |
| | 1,3,4-thiadiazol-2-yl)pyridin-4-yl)- |
| | carbamate (Preparation 425) 90 mg, yellow |
| | solid. ¹H NMR (400 MHz, DMSO-d₆) δ: |
| | ppm 8.66 (br s, 2H), 8.38 (s, 1H), 7.01 (s, |
| | 1H), 2.79 (s, 3H) , 2.18 (s, 3H). |
| 468 | 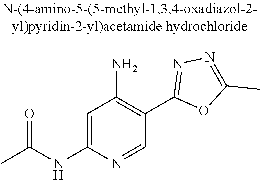 [SMILES: CC(=O)Nc1cc(N)c(-c2nnc(C)o2)cn1] |
| | SM: tert-butyl (2-acetamido-5-(5-methyl- |
| | 1,3,4-oxadiazol-2-yl)pyridin-4-yl)- |
| | carbamate (Preparation 426) 70 mg, |
| | yellow solid. |
| 469 | 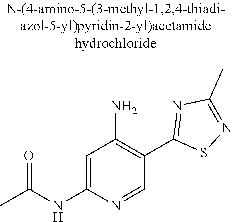 [SMILES: CC(=O)Nc1cc(N)c(-c2nc(C)ns2)cn1] |
| | SM: tert-butyl (2-acetamido-5-(3- |
| | methyl-1,2,4-thiadiazol-5-yl)pyridin- |
| | 4-yl)carbamate (Preparation 427) |
| | 62.8 mg as white solid. LCMS |
| | m/z =250.2 [M + H]⁺ |
| 470 | 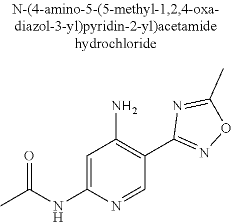 [SMILES: CC(=O)Nc1cc(N)c(-c2noc(C)n2)cn1] |
| | SM: tert-butyl (2-acetamido-5-(5- |
| | methyl-1,2,4-oxadiazol-3-yl)pyridin- |
| | 4-yl)carbamate (Preparation 438) |
| | 210 mg, crude. ¹H NMR (400 MHz, |
| | DMSO-d₆) δ: ppm 12.00 (s, 1H), |
| | 8.64 (s, 1H), 6.93 (s, 1H), 2.70 (s, |
| | 3H), 2.22 (s, 3H). |

### Preparation 471

### N-(4-amino-5-(6-methylpyridazin-3-yl)pyridin-2-yl)acetamide hydrochloride

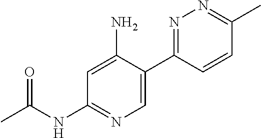 [SMILES: CC(=O)Nc1cc(N)c(-c2ccc(C)nn2)cn1]

A solution of tert-butyl (2-acetamido-5-(6-methylpyridazin-3-yl)pyridin-4-yl)carbamate (Preparation 430, 160 mg, 0.465 mmol) in dioxane (4 mL) and HCl (4 M in dioxane, 1 mL) was stirred for 1 h, then the heterogeneous mixture was heated to 50° C. and stirred for 4 days. The reaction was cooled to rt then diluted with EtOAc. The mixture was filtered, the filter cake was rinsed with EtOAc to afford a tan solid as N-(4-amino-5-(6-methylpyridazin-3-yl)pyridin-2-yl)acetamide hydrochloride (161 mg). LCMS m/z=243.9 [M+H]⁺.

### Preparation 473

### N-(4-amino-5-(6-methoxypyridazin-3-yl)pyridin-2-yl)acetamide-2,2,2-d3 trifluoroacetate

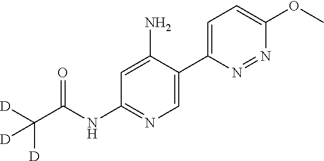 [SMILES: [2H]C([2H])([2H])C(=O)Nc1cc(N)c(-c2ccc(OC)nn2)cn1]

To a vial containing tert-butyl (2-(acetamido-2,2,2-d3)-5-(6-methoxypyridazin-3-yl)pyridin-4-yl)carbamate (Preparation 442, 124 mg, 0.343 mmol) in DCM (2 mL) was added TFA (0.2 mL, 2.61 mmol) and the reaction was stirred at 23° C. for 23 h. The mixture was concentrated under reduced pressure to afford N-(4-amino-5-(6-methoxypyridazin-3-yl)pyridin-2-yl)acetamide-2,2,2-d3 trifluoroacetate as an off-white residue (132 mg, crude). LCMS m/z=263.1 [M+H]⁺.

### Preparation 474

### N-(4-amino-5-(5-methyl-4,5,6,7-tetrahydropyrazolo[1,5-a]pyrazin-2-yl)pyridin-2-yl)acetamide trifluoroacetate

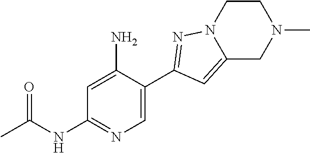 [SMILES: CC(=O)Nc1cc(N)c(-c2cc3n(n2)CCN(C)C3)cn1]

N-(4-amino-5-(5-methyl-4,5,6,7-tetrahydropyrazolo[1,5-a]pyrazin-2-yl)pyridin-2-yl)acetamide trifluoroacetate was obtained as an orange oil from tert-butyl (2-acetamido-5-(5-methyl-4,5,6,7-tetrahydropyrazolo[1,5-a]pyrazin-2-yl)pyridin-4-yl)carbamate (Preparation 432) following the procedure described in Preparation 473. LCMS m/z=287.1 [M+H]⁺.

### Preparation 475

### N-(4-amino-5-(1-methyl-6-oxo-1,6-dihydropyridazin-3-yl)pyridin-2-yl)acetamide

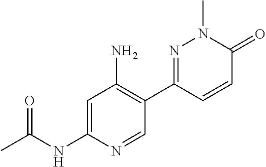 [SMILES: CC(=O)Nc1cc(N)c(-c2ccc(=O)n(C)n2)cn1]

To a solution of tert-butyl (2-acetamido-5-(1-methyl-6-oxo-1,6-dihydropyridazin-3-yl)pyridin-4-yl)carbamate (Preparation 422, 160 mg, 0.445 mmol) in HFiPA (2 mL) was added TFA (595.6 mg, 5.2 mmol) and the reaction mixture was stirred at 25° C. for 12 h. The reaction was slowly quenched with ammonia until pH=7-8. The reaction mixture was concentrated under reduced pressure and the residue was purified by Prep-HPLC (Method B, 3-30%) to give
N-(4-amino-5-(1-methyl-6-oxo-1,6-dihydropyridazin-3-yl)pyridin-2-yl)acetamide (80 mg, 69.3% yield) as a white solid. LCMS m/z=260.1 [M+H]⁺

### Preparation 476

### N-(4-amino-5-(1-methyl-2-oxo-1,2-dihydropyrimidin-4-yl)pyridin-2-yl)acetamide trifluoroacetate

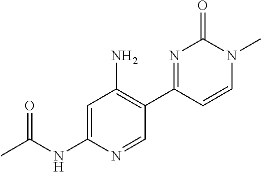 [SMILES: CC(=O)Nc1cc(N)c(-c2ccn(C)c(=O)n2)cn1]

To a solution of tert-butyl (2-acetamido-5-(1-methyl-2-oxo-1,2-dihydropyrimidin-4-yl)pyridin-4-yl)carbamate (Preparation 424, 120 mg, 0.33 mmol) in HFIPA (3 mL) was added TFA (0.6 mL) and the mixture was stirred at 25° C. for 2 h. The mixture was concentrated under reduced pressure to give N-(4-amino-5-(1-methyl-2-oxo-1,2-dihydropyrimidin-4-yl)pyridin-2-yl)acetamide trifluoroacetate (120 mg, crude) as white solid. LCMS m/z=260.2 [M+H]⁺

### Preparation 477

### N-(4′-amino-5-((difluoromethoxy)methyl)-[2,3′-bipyridin]-6′-yl)acetamide hydrochloride

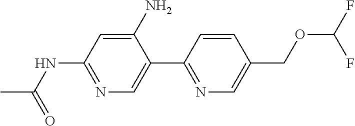 [SMILES: CC(=O)Nc1cc(N)c(-c2ccc(COC(F)F)cn2)cn1]

A mixture of tert-butyl (2-acetamido-5-(4,4,5,5-tetramethyl-1,3,2-dioxaborolan-2-yl)pyridin-4-yl)carbamate (Preparation 421, 500 mg, 0.663 mmol), 2-chloro-5-((difluoromethoxy)methyl)pyridine (Preparation 182, 192 mg, 0.994 mmol), Pd(PCy₃)₂ (44 mg, 66 mol) and aq. K₂CO₃ (2 M, 1.99 mmol, 0.994 mL) in dioxane (5.0 mL) was heated at 100° C. for 2 h. The cooled mixture was filtered, the filtrate was diluted with EtOAc, washed with water, then brine. The organic layer was dried and concentrated. The crude was purified by chromatography on silica gel (0-70% EtOAc-EtOH 3:1 with 2% NH₄OH in heptane) to give tert-butyl (6′-acetamido-5-((difluoromethoxy)methyl)-[2,3′-bipyridin]-4′-yl)carbamate (91 mg) (m/z: [M+H]+=409) as a white powder which was then dissolved in DCM (5.0 mL). HCl (4 M, 4.00 mmol, 1.00 mL) was then added and the mixture stirred at rt overnight. The mixture was concentrated in vacuo and the product dried under vacuum to give N-(4′-amino-5-((difluoromethoxy)methyl)-[2,3′-bipyridin]-6′-yl)acetamide hydrochloride (81 mg). LCMS m/z=[M+H]⁺

### Preparation 478

### tert-butyl (5-bromo-2-chloropyridin-4-yl)carbamate

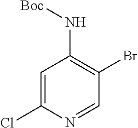 [SMILES: CC(C)(C)OC(=O)Nc1cc(Cl)ncc1Br]

To a mixture of 5-bromo-2-chloropyridin-4-amine (20 g, 96.41 mmol) in DCM (150 mL) was added TEA (29.27 g, 289.2 mmol), Boc₂O (25.25 g, 115.7 mmol) and DMAP (1.18 g, 9.64 mmol) and the mixture was stirred at 20° C. for 12 h under N₂. The mixture was concentrated and then the residue diluted with water (200 mL). The mixture was extracted with EtOAc (150 mL), the combined organic layers were washed with brine (150 mL), dried over Na₂SO₄, filtered and concentrated. The crude material was purified by chromatography (PE/EtOAc=5/1 to 2/1) to yield tert-butyl (5-bromo-2-chloropyridin-4-yl)carbamate (21 g, 70.8% yield) as a white solid. 1H NMR: (400 MHz, CDCl₃) δ ppm 8.37 (s, 1H), 8.27 (s, 1H), 1.57 (s, 9H).

### Preparation 479

### tert-butyl (2-chloro-5-(1-methyl-1H-pyrazol-3-yl)pyridin-4-yl)carbamate

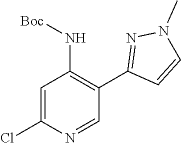 [SMILES: Cn1ccc(-c2cnc(Cl)cc2NC(=O)OC(C)(C)C)n1]

To a mixture of tert-butyl (5-bromo-2-chloropyridin-4-yl)carbamate (Preparation 478, 16 g, 52.0 mmol) in dioxane (200 mL) and water (20 mL) was added 1-methyl-3-(4,4,5,5-tetramethyl-1,3,2-dioxaborolan-2-yl)-1H-pyrazole (10.82 g, 52.0 mmol), K₃PO₄ (5.52 g, 26.0 mmol) and Pd(dppf)Cl₂ (3.81 g, 5.20 mmol) and the reaction was stirred at 100° C. for 12 h under N₂. The cooled mixture was concentrated and the residue was diluted with water (200 mL). The resulting solution was extracted with EtOAc (150 mL×3) and the combined organic layers were washed with brine (200 mL) and dried over anhydrous Na₂SO₄. The solvent was removed in vacuo and the residue was purified by silica gel column chromatography (PE/EtOAc=10:1 to 3:1) to give tert-butyl (2-chloro-5-(1-methyl-1H-pyrazol-3-yl)pyridin-4-yl)carbamate (8.6 g, 53.5% yield) as a yellow solid. ¹H NMR: (400 MHz, CDCl₃) δ ppm 10.87 (s, 1H), 8.53 (s, 1H), 8.41 (s, 1H), 7.47 (d, J=2.4 Hz, 1H), 6.65 (d, J=2.0 Hz, 1H), 4.01 (s, 3H), 1.58 (s, 9H).

### Preparation 480

### tert-butyl (2-acetamido-5-(1-methyl-1H-pyrazol-3-yl)pyridin-4-yl)carbamate

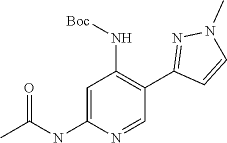

To a mixture of tert-butyl (2-chloro-5-(1-methyl-1H-pyrazol-3-yl)pyridin-4-yl)carbamate (Preparation 479, 8.6 g, 27.9 mmol) in dioxane (150 mL) was added acetamide (3.29 g, 55.71 mmol), Cs₂CO₃ (27.23 g, 83.7 mmol), Xantphos (3.22 g, 5.57 mmol) and Pd₂(dba)₃ (2.55 g, 2.79 mmol) and the reaction mixture was stirred at 110° C. for 12 h under N₂. The cooled mixture was concentrated in vacuo and the residue was diluted with water (100 mL). The resulting solution was extracted with EtOAc (80 mL×3), the combined organic layers were washed with brine (100 mL) and dried over anhydrous Na₂SO₄. The solvent was removed in vacuo and the residue was purified by silica gel column chromatography (PE/EtOAc=1/1 to 0/1) to give tert-butyl (2-acetamido-5-(1-methyl-1H-pyrazol-3-yl)pyridin-4-yl)carbamate (7 g, 75.8% yield) as a yellow solid.

### Preparation 481

### N-(4-amino-5-(1-methyl-1H-pyrazol-3-yl)pyridin-2-yl)acetamide

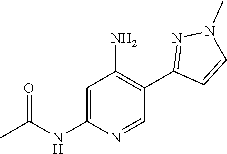 [SMILES: CC(=O)Nc1cc(N)c(-c2ccn(C)n2)cn1]

To a mixture of tert-butyl (2-acetamido-5-(1-methyl-1H-pyrazol-3-yl)pyridin-4-yl)carbamate (Preparation 480, 6 g, 18.11 mmol) in DCM (50 mL) was added TFA (23.6 mL) and the reaction stirred at 20° C. for 3 h. The solvent was removed in vacuo and the residue was diluted with water, then sat. NaHCO₃ was added to adjust the pH to 11. The resulting mixture was extracted with EtOAc (50 mL×3) and the combined organic layers were dried over Na₂SO₄ and filtered. The filtrate was concentrated and the residue purified by Prep-HPLC (Method A, Gradient 15-45%) to give N-(4-amino-5-(1-methyl-1H-pyrazol-3-yl)pyridin-2-yl)acetamide (2 g, 47.8% yield) as a yellow solid. LCMS m/z=232.2 [M+H]⁺

### Preparation 482

### N-(4-amino-5-bromopyridin-2-yl)acetamide hydrochloride

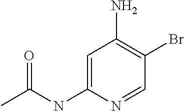

To a solution of tert-butyl (2-acetamido-5-bromopyridin-4-yl)carbamate (Preparation 53, 10 g, 30.29 mmol) in DCM (80 mL) was added HCl/dioxane (4 M, 15 mL) and the mixture was stirred at 25° C. for 16 h under N₂. The mixture was concentrated under reduced pressure to give N-(4-amino-5-bromopyridin-2-yl)acetamide hydrochloride (9.0 g, crude) as a white solid. LCMS m/z=229.9 [M+H]⁺

### Preparation 483

### N-(5-bromo-4-((2-(1,1-difluoroethyl)-6-methylpyrimidin-4-yl)amino)pyridin-2-yl)acetamide

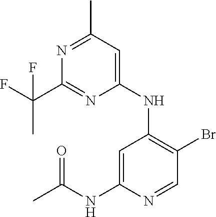 [SMILES: CC(=O)Nc1cc(Nc2cc(C)nc(C(C)(F)F)n2)c(Br)cn1]

To a solution of N-(4-amino-5-bromopyridin-2-yl)acetamide hydrochloride (Preparation 482, 5 g, 18.76 mmol) in dioxane (50 mL) was added 4-chloro-2-(1,1-difluoroethyl)-6-methylpyrimidine (Preparation 92, 3.97 g, 20.64 mmol), Xantphos (1.09 g, 1.88 mmol), K₃PO₄ (7.96 g, 37.52 mmol) and Pd₂(dba)₃ (1.72 g, 1.88 mmol) and the mixture was stirred at 100° C. for 2 h under N₂. The reaction mixture was filtered and concentrated in vacuo. The mixture was diluted with water (500 mL) and extracted with EtOAc (55 mL×3). The combined organic layers were washed with brine (55 mL), dried over Na₂SO₄, filtered and concentrated. The crude product was purified by silica gel column chromatography (PE/EtOAc=1/0 to 1/2) to give N-(5-bromo-4-((2-(1,1-difluoroethyl)-6-methylpyrimidin-4-yl)amino)pyridin-2-yl)acetamide (4.56 g, 62.9% yield) as a white solid. ¹H NMR (400 MHz, DMSO-d₆) δ: ppm 10.52 (s, 1H), 9.33 (s, 1H), 8.79 (s, 1H), 8.40 (s, 1H), 7.05 (s, 1H), 2.40 (s, 3H), 2.07 (s, 3H), 1.98 (t, J=19.0 Hz, 3H).

### Preparation 484

### N-(5-bromo-4-((2-(1,1-difluoroethyl)pyrimidin-4-yl)amino)pyridin-2-yl)acetamide

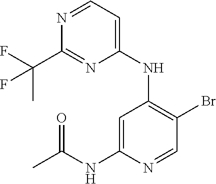 [SMILES: CC(=O)Nc1cc(Nc2ccnc(C(C)(F)F)n2)c(Br)cn1]

N-(5-Bromo-4-((2-(1,1-difluoroethyl)pyrimidin-4-yl)amino)pyridin-2-yl)acetamide was obtained as a white solid, 1.93 g, 69.1% yield, from N-(4-amino-5-bromopyridin-2-yl)acetamide hydrochloride (Preparation 482) and 4-chloro-2-(1,1-difluoroethyl)pyrimidine (Preparation 148), following the procedure described in Preparation 483. 1H NMR (400 MHz, DMSO-d₆) δ: ppm 10.59 (s, 1H), 9.52 (s, 1H), 8.83 (s, 1H), 8.56 (d, J=5.6 Hz, 1H), 8.44 (s, 1H), 7.25 (d, J=6.0 Hz, 1H), 2.08 (s, 3H), 2.00 (t, J=19.2 Hz, 3H).

### Preparation 485

### N-(4-((2-(1,1-difluoroethyl)pyrimidin-4-yl)amino)-5-(4,4,5,5-tetramethyl-1,3,2-dioxaborolan-2-yl)pyridin-2-yl)acetamide

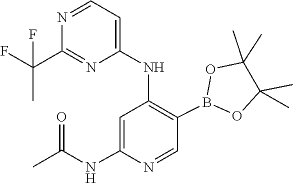 [SMILES: CC(=O)Nc1cc(Nc2ccnc(C(C)(F)F)n2)c(B2OC(C)(C)C(C)(C)O2)cn1]

To a microwave vial, N-(5-bromo-4-((2-(1,1-difluoroethyl)pyrimidin-4-yl)amino)pyridin-2-yl)acetamide (Preparation 484, 500 mg, 1.34 mmol), KOAc (527 mg, 5.37 mmol) and (BPin)₂ (750 mg, 2.96 mmol) were added, followed by 2-MeTHF (12 mL). In a different vial, XPhos (64 mg, 134 mol) and Pd(OAc)₂ (15 mg, 67 mol) were suspended in 2-MeTHF (4 mL) and both vials stirred for 30 min under N₂. The solution of catalyst was added to the first vial and the reaction was heated at 75° C. overnight. The cooled reaction mixture was filtered through a plug of Celite®, rinsing through with 2-MeTHF. The filtrate was concentrated and diluted with MeCN. The solid formed was filtered off, and the filtrate was concentrated in vacuo to give N-(4-((2-(1,1-difluoroethyl)pyrimidin-4-yl)amino)-5-(4,4,5,5-tetramethyl-1,3,2-dioxaborolan-2-yl)pyridin-2-yl)acetamide crude (920 mg).

### Preparation 486

### N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide

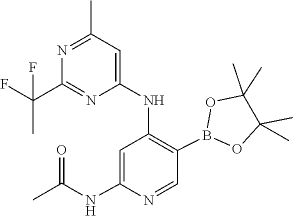 [SMILES: CC(=O)Nc1cc(Nc2cc(C)nc(C(C)(F)F)n2)c(B2OC(C)(C)C(C)(C)O2)cn1]

The title compound was synthesized in the similar way as described in Example 15, start with
N-(5-bromo-4-((2-(1,1-difluoroethyl)-6-methylpyrimidin-4-yl)amino)pyridin-2-yl)acetamide (Preparation 483) to give the title compound as a crude which was used in the next step without further purification. LCMS m/z=352 [M+H]⁺ (boronic acid)

### Preparation 487

### (6-acetamido-4-((2-(1,1-difluoroethyl)pyrimidin-4-yl)amino)pyridin-3-yl)boronic acid

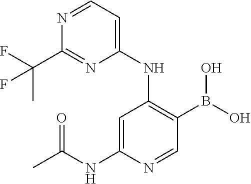 [SMILES: CC(=O)Nc1cc(Nc2ccnc(C(C)(F)F)n2)c(B(O)O)cn1]

To a solution of N-(5-bromo-4-((2-(1,1-difluoroethyl)pyrimidin-4-yl)amino)pyridin-2-yl)acetamide (Preparation 484, 1.0 g, 2.69 mmol) in dioxane (10 mL) was added (BPin)₂ (1.71 g, 6.72 mmol), cataCXiumA Pd G3 (195.7 mg, 0.269 mmol), KOAc (1.05 g, 10.75 mmol) and the mixture was stirred at 90° C. for 2 h under N₂. The mixture was concentrated under reduced pressure to give (6-acetamido-4-((2-(1,1-difluoroethyl)pyrimidin-4-yl)amino)pyridin-3-yl)boronic acid (3.2 g, crude) as black solid. LCMS m/z=338.0 [M+H]⁺

### Preparation 488

### (6-acetamido-4-((2-(1,1-difluoroethyl)-6-methylpyrimidin-4-yl)amino)pyridin-3-yl)boronic acid

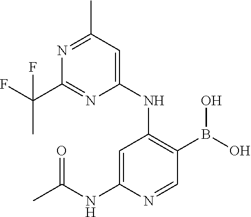 [SMILES: CC(=O)Nc1cc(Nc2cc(C)nc(C(C)(F)F)n2)c(B(O)O)cn1]

To a solution of N-(5-bromo-4-((2-(1,1-difluoroethyl)-6-methylpyrimidin-4-yl)amino)pyridin-2-yl)acetamide (Preparation 483, 3 g, 7.77 mmol) in dioxane (40 mL) was added (BPin)₂ (4.93 g, 19.42 mmol), KOAc (3.05 g, 31.1 mmol) and Pd(dppf)Cl₂ (568.4 mg, 0.777 mmol) and the mixture was stirred at 90° C. for 2 h under N₂. The mixture was concentrated under reduced pressure to give (6-acetamido-4-((2-(1,1-difluoroethyl)-6-methylpyrimidin-4-yl)amino)pyridin-3-yl)boronic acid (9.8 g, crude) as a black solid. LCMS m/z=352.0 [M+H]⁺

### Preparation 489

### N-(5-bromo-4-((2-(1,1-difluoroethyl)-6-ethylpyrimidin-4-yl)amino)pyridin-2-yl)acetamide

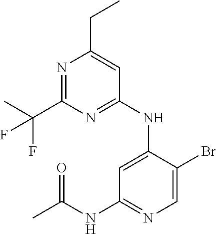 [SMILES: CCc1cc(Nc2cc(NC(C)=O)ncc2Br)nc(C(C)(F)F)n1]

To a solution of N-(4-amino-5-bromopyridin-2-yl)acetamide hydrochloride (Preparation 482, 500 mg, 1.88 mmol) in dioxane (6 mL) was added 4-chloro-2-(1,1-difluoroethyl)-6-ethylpyrimidine (Preparation 94, 465 mg, 2.25 mmol), Xantphos (217.1 mg, 0.375 mmol), Pd₂(dba)₃ (171.8 mg, 0.188 mmol), K₃PO₄ (796 mg, 3.75 mmol) and the mixture stirred at 100° C. for 2 h under N₂. The reaction mixture was concentrated under reduced pressure and the residue diluted with H₂O (50 mL) and extracted with EtOAc (30 mL×3). The combined organics were washed with brine (50 mL), dried (Na₂SO₄), concentrated and the residue purified on silica gel column chromatography (0-50% EtOAc/PE) to give N-(5-bromo-4-((2-(1,1-difluoroethyl)-6-ethylpyrimidin-4-yl)amino)pyridin-2-yl)acetamide as a white solid (577.5 mg, 77%). ¹H NMR (400 MHz, CDCl₃) δ ppm: 9.03 (s, 1H), 8.29 (s, 1H), 7.91 (br s, 1H), 7.38 (s, 1H), 6.94 (s, 1H), 2.84 (q, J=7.6 Hz, 2H), 2.22 (s, 3H), 2.13 (t, J=18.8 Hz, 3H), 1.35 (t, J=7.6 Hz, 3H).

### Preparation 490

### (6-acetamido-4-((2-(1,1-difluoroethyl)-6-ethylpyrimidin-4-yl)amino)pyridin-3-yl)boronic acid

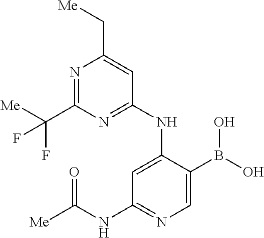 [SMILES: CCc1cc(Nc2cc(NC(C)=O)ncc2B(O)O)nc(C(C)(F)F)n1]

To a solution of N-(5-bromo-4-((2-(1,1-difluoroethyl)-6-ethylpyrimidin-4-yl)amino)pyridin-2-yl)acetamide (Preparation 489, 550 mg, 1.37 mmol) in dioxane (5 mL) was added (BPin)₂ (698 mg, 2.75 mmol), KOAc (540 mg, 5.50 mmol), cataCXIumA Pd G3 (100 mg, 0.14 mmol) and the mixture stirred at 100° C. for 2 h under N2. The mixture was concentrated under reduced pressure to give (6-acetamido-4-((2-(1,1-difluoroethyl)-6-ethylpyrimidin-4-yl)amino)pyridin-3-yl)boronic acid as a black solid (1.2 g, crude) as black solid. LCMS m/z=366.2 [M+H]+.

### Preparation 491

### N-(4-((2-(1,1-difluoroethyl)-6-ethylpyrimidin-4-yl)amino)-5-(4,4,5,5-tetramethyl-1,3,2-dioxaborolan-2-yl)pyridin-2-yl)acetamide

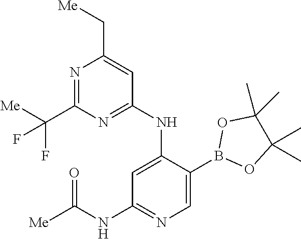 [SMILES: CCc1cc(Nc2cc(NC(C)=O)ncc2B2OC(C)(C)C(C)(C)O2)nc(C(C)(F)F)n1]

A vial containing N-(5-bromo-4-((2-(1,1-difluoroethyl)-6-ethylpyrimidin-4-yl)amino)pyridin-2-yl)acetamide (Preparation 489, 1 g, 2.50 mmol), (BPin)₂ (1.40 g, 5.50 mmol), XPhos Pd G3 (266.3 mg, 0.315 mmol), and KOAc (986.51 mg, 10.05 mmol) in anhydrous 2-MeTHF (12 mL) was degassed then backfilled with N₂ and heated at 75° C. for 4 h. The cooled mixture was diluted with EtOAc and washed with brine, dried and concentrated in vacuo to give N-(4-((2-(1,1-difluoroethyl)-6-ethylpyrimidin-4-yl)amino)-5-(4,4,5,5-tetramethyl-1,3,2-dioxaborolan-2-yl)pyridin-2-yl)acetamide (2.3 g, crude). LCMS m/z=366.2 [Boronic acid]

### Preparation 492

### methyl 6-acetamido-4-aminonicotinate

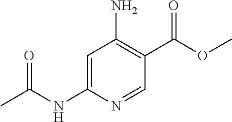

To a solution of methyl 4-amino-6-chloronicotinate (500 mg, 2.68 mmol) in dioxane (15 mL) was added acetamide (633.1 mg, 10.72 mmol), Cs₂CO₃ (2.6 g, 8.04 mmol) and BrettPhos Pd G3 (485.8 mg, 0.536 mmol) and the reaction mixture was stirred at 90° C. for 16 h under N₂. The mixture was diluted with water (15 mL) and extracted with EtOAc (20 mL×3). The combined organic phase was washed with brine (60 mL), dried over anhydrous Na₂SO₄, filtered and concentrated. The crude was purified by column chromatography (PE/EtOAc=1/0 to 1/1) to give methyl 6-acetamido-4-aminonicotinate (290.2 mg, 51.8% yield) as a yellow solid. LCMS m/z=210.2 [M+H]⁺

### Preparation 493

### methyl 6-acetamido-4-((2-(1,1-difluoroethyl)-6-ethylpyrimidin-4-yl)amino)nicotinate

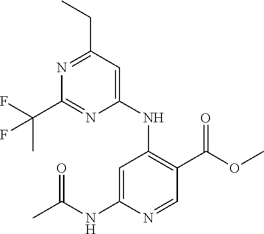 [SMILES: CCc1cc(Nc2cc(NC(C)=O)ncc2C(=O)OC)nc(C(C)(F)F)n1]

To a solution of methyl 6-acetamido-4-aminonicotinate (Preparation 492, 299.0 mg, 1.43 mmol) in dioxane (10 mL) was added 4-chloro-2-(1,1-difluoroethyl)-6-ethylpyrimidine (Preparation 94, 324.8 mg, 1.57 mmol), Pd₂(dba)₃ (130.88 mg, 0.143 mmol), Xantphos (165.4 mg, 0.286 mmol) and Cs₂CO₃ (1.4 g, 4.29 mmol) and the reaction was stirred at 70° C. for 2 h under N₂. The mixture was diluted with water (15 mL) and extracted with EtOAc (20 mL×3). The combined organic phase was washed with brine (60 mL), dried over anhydrous Na₂SO₄, filtered and concentrated. The crude was purified by column chromatography (PE/EtOAc=1/0 to 1/1) to give methyl 6-acetamido-4-((2-(1,1-difluoroethyl)-6-ethylpyrimidin-4-yl)amino)nicotinate (499.5 mg, 92.1% yield) as a light-yellow solid. LCMS m/z=380.2 [M+H]⁺

### Preparation 494

### 6-acetamido-4-((2-(1,1-difluoroethyl)-6-ethylpyrimidin-4-yl)amino)nicotinic acid

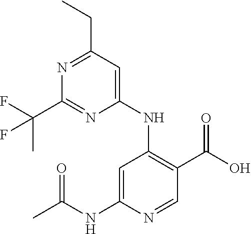 [SMILES: CCc1cc(Nc2cc(NC(C)=O)ncc2C(=O)O)nc(C(C)(F)F)n1]

To a solution of methyl 6-acetamido-4-((2-(1,1-difluoroethyl)-6-ethylpyrimidin-4-yl)amino)nicotinate (Preparation 493, 100 mg, 0.264 mmol) in THF (4 mL) and water (2 mL) was added LiOH·H₂O (31.6 mg, 1.32 mmol) and the reaction stirred at 25° C. for 1 h. The mixture was concentrated and adjusted to pH=5 using HCl (4 M). The mixture was diluted with water (15 mL) and extracted with EtOAc (20 mL×3). The combined organic phase was washed with brine (60 mL) and concentrated to give 6-acetamido-4-((2-(1,1-difluoroethyl)-6-ethylpyrimidin-4-yl)amino)nicotinic acid (100.0 mg, crude) as a white solid. LCMS m/z=366.0 [M+H]⁺

### Preparation 495

### 2-chloro-5-(4,4,5,5-tetramethyl-1,3,2-dioxaborolan-2-yl)pyridin-4-amine

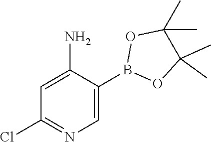 [SMILES: CC1(C)OB(c2cnc(Cl)cc2N)OC1(C)C]

To a solution of 5-bromo-2-chloropyridin-4-amine (4 g, 19.28 mmol) and (BPin)₂ (14.69 g, 57.84 mmol) in dioxane (50 mL) was added KOAc (3.78 g, 38.56 mmol) and Pd(dppf)Cl₂ (1.41 g, 1.93 mmol) under N₂ and the reaction was stirred at 80° C. for 12 h under N₂. The mixture was diluted with water (50 mL), extracted with EtOAc (50 mL×3), dried over Na₂SO₄, filtered and concentrated under reduced pressure to give 2-chloro-5-(4,4,5,5-tetramethyl-1,3,2-dioxaborolan-2-yl)pyridin-4-amine (1.4 g, crude) as brown oil.

### Preparation 496

### 2-chloro-5-(2,2-dimethyl-2,3-dihydrobenzo[b][1,4]dioxin-6-yl)pyridin-4-amine

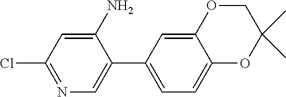 [SMILES: CC1(C)COc2cc(-c3cnc(Cl)cc3N)ccc2O1]

To a solution of 2-chloro-5-(4,4,5,5-tetramethyl-1,3,2-dioxaborolan-2-yl)pyridin-4-amine (Preparation 495, 1.4 g, 8.12 mmol) and 6-bromo-2,2-dimethyl-2,3-dihydro-[1,4]dioxino[2,3-b]pyridine (Preparation 288, 2.20 g, 8.12 mmol) in dioxane (40 mL) and H₂O (10 mL) was added Pd(dppf)Cl₂ (1.01 g, 1.38 mmol) and K₃PO₄ (2.93 g, 13.81 mmol) and the reaction mixture was stirred at 75° C. for 2 h under N₂ atmosphere. The mixture was diluted with water (100 mL), extracted with EtOAc (100 mL×3), dried over Na₂SO₄, filtered and concentrated under reduced pressure. The residue was purified by silica gel chromatography (PE/EtOAc=100/1 to 1/1) to give 2-chloro-5-(2,2-dimethyl-2,3-dihydrobenzo[b][1,4]dioxin-6-yl)pyridin-4-amine (1.5 g, 63.3% yield) as white solid. 1H NMR: (400 MHz, DMSO-d₆) δ ppm 8.31 (s, 1H), 9.14 (s, 1H), 7.36-7.47 (m, 4H), 6.69 (s, 1H), 4.17 (s, 2H), 1.33 (s, 6H).

### Preparation 497

### 2-chloro-5-(2,2-dimethyl-2,3-dihydrobenzo[b][1,4]dioxin-6-yl)pyridin-4-amine

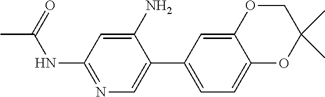 [SMILES: CC(=O)Nc1cc(N)c(-c2ccc3c(c2)OCC(C)(C)O3)cn1]

2-Chloro-5-(2,2-dimethyl-2,3-dihydrobenzo[b][1,4]dioxin-6-yl)pyridin-4-amine was obtained as a brown solid, 6.3 g, 87.7%, from 2-chloro-5-(2,2-dimethyl-2,3-dihydrobenzo[b][1,4]dioxin-6-yl)pyridin-4-amine (Preparation 496) and acetamide, following a similar procedure to that described in Preparation 480. LCMS m/z=314.9 [M+H]⁺.

### Preparation 498

### 2-chloro-5-(1-methyl-1H-pyrazol-3-yl)pyridin-4-amine

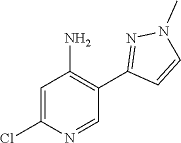

To a solution of 2-chloro-5-iodopyridin-4-amine (1 g, 3.93 mmol) in dioxane (45 mL) and H₂O (5 mL) was added 1-methyl-3-(4,4,5,5-tetramethyl-1,3,2-dioxaborolan-2-yl)-1H-pyrazole (1.64 g, 7.86 mmol), Pd(dppf)Cl₂ (287.6 mg, 0.393 mmol) and K₂CO₃ (1.09 g, 7.86 mmol). The reaction was stirred at 100° C. for 12 h under N₂. The mixture was diluted with H₂O (30 ml) and extracted with EtOAc (20 ml×3), dried over Na₂SO₄, filtered and concentrated under reduced pressure. The residue was purified by column chromatography (PE/EtOAc=100/1 to 4/1) to give 2-chloro-5-(1-methyl-1H-pyrazol-3-yl)pyridin-4-amine (576 mg, 70.3% yield) as a brown solid. LCMS m/z=209.2 [M+H]⁺

### Preparation 499

### 4′-amino-6′-chloro-[2,3′-bipyridine]-5-carbonitrile

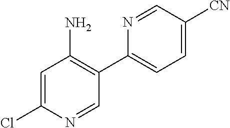 [SMILES: N#Cc1ccc(-c2cnc(Cl)cc2N)nc1]

To a solution of 2-chloro-5-iodopyridin-4-amine (1 g, 3.93 mmol) in toluene (5.0 mL), MeOH (5.0 mL) and H₂O (0.5 mL) was added 6-bromonicotinonitrile (863.0 mg, 4.72 mmol), (BPin)₂ (2.0 g, 7.86 mmol) Pd(OAc)₂ (176.5 mg, 0.786 mmol), CsF (1.6 g, 23.58 mmol) and bis(1-adamantyl)-butyl-phosphane (281.8 mg, 0.786 mmol) and the reaction was stirred at 100° C. for 2 h under N₂. The reaction mixture was concentrated under reduced pressure and the residue partitioned between H₂O (15 mL) and EtOAc (15 mL) and the layers separated. The aqueous phase was extracted with EtOAc (15 mL×3), the combined organic extracts dried over Na₂SO₄, filtered and concentrated. The residue was purified by silica gel chromatography (EtOAc/PE) to give 4′-amino-6′-chloro-[2,3′-bipyridine]-5-carbonitrile (500 mg, crude) as a yellow solid. LCMS m/z=231.0 [M+H]⁺

### Preparation 500

### 6′-chloro-5-fluoro-[2,3′-bipyridin]-4′-amine

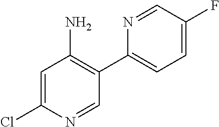 [SMILES: Nc1cc(Cl)ncc1-c1ccc(F)cn1]

6′-Chloro-5-fluoro-[2,3′-bipyridin]-4′-amine was obtained as a brown solid, 2.5 g, 94.8%, from 2-chloro-5-iodopyridin-4-amine and 2-bromo-5-fluoropyridine, following a similar procedure to that described in Preparation 499. ¹H NMR (400 MHz, CDCl₃) δ ppm 8.47 (d, J=2.8 Hz, 1H), 8.42 (s, 1H), 7.73-7.77 (m, 1H), 7.55-7.56 (m, 1H), 6.62-6.67 (m, 3H).

### Preparation 501

### N-(2-chloro-5-(1-methyl-1H-pyrazol-3-yl)pyridin-4-yl)-2-(1,1-difluoroethyl)-6-methylpyrimidin-4-amine

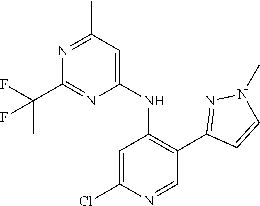 [SMILES: Cc1cc(Nc2cc(Cl)ncc2-c2ccn(C)n2)nc(C(C)(F)F)n1]

To a solution of 2-chloro-5-(1-methyl-1H-pyrazol-3-yl)pyridin-4-amine (Preparation 498, 500 mg, 2.40 mmol) in dioxane (35 mL) was added 4-chloro-2-(1,1-difluoroethyl)-6-methylpyrimidine (Preparation 92, 923.1 mg, 4.79 mmol), Pd₂(dba)₃ (219.44 mg, 0.24 mmol), Xantphos (138.7 mg, 0.24 mmol) and K₃PO₄ (1.02 g, 4.79 mmol) and the reaction mixture was stirred at 100° C. for 16 h under N₂. The mixture was diluted with H₂O (50 ml), extracted with EtOAc (30 ml×3), dried over Na₂SO₄, filtered and concentrated under reduced pressure. The residue was purified by column chromatography (PE/EtOAc=100/1 to 2/1) to give N-(2-chloro-5-(1-methyl-1H-pyrazol-3-yl)pyridin-4-yl)-2-(1,1-difluoroethyl)-6-methylpyrimidin-4-amine (438 mg, 50.1% yield) as a yellow solid. LCMS m/z=365.2 [M+H]⁺

### Preparation 502

### 6′-chloro-4′-((2-(1,1-difluoroethyl)-6-methylpyrimidin-4-yl)amino)-[2,3′-bipyridine]-5-carbonitrile

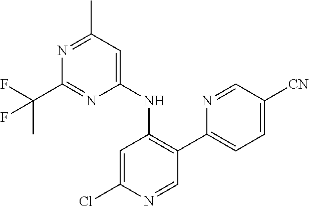 [SMILES: Cc1cc(Nc2cc(Cl)ncc2-c2ccc(C#N)cn2)nc(C(C)(F)F)n1]

6′-Chloro-4′-((2-(1,1-difluoroethyl)-6-methylpyrimidin-4-yl)amino)-[2,3′-bipyridine]-5-carbonitrile was obtained as a yellow solid, 360 mg, crude from 4′-amino-6′-chloro-[2,3′-bipyridine]-5-carbonitrile (Preparation 499) and 4-chloro-2-(1,1-difluoroethyl)pyrimidine (Preparation 148), following the procedure described in Preparation 501. LCMS m/z=387.1 [M+H]⁺

### Preparation 503

### 6′-chloro-N-(2-(1,1-difluoroethyl)-6-methylpyrimidin-4-yl)-5-fluoro-[2,3′-bipyridin]-4′-amine

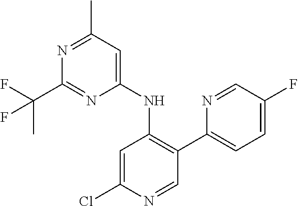 [SMILES: Cc1cc(Nc2cc(Cl)ncc2-c2ccc(F)cn2)nc(C(C)(F)F)n1]

6′-Chloro-N-(2-(1,1-difluoroethyl)-6-methylpyrimidin-4-yl)-5-fluoro-[2,3′-bipyridin]-4′-amine was obtained as a yellow solid, 320 mg, 18.8%, from 6′-chloro-5-fluoro-[2,3′-bipyridin]-4′-amine (Preparation 500) and 4-chloro-2-(1,1-difluoroethyl)pyrimidine (Preparation 148), following the procedure described in Preparation 502. 1H NMR (400 MHz, CDCl₃) δ ppm 12.19 (s, 1H), 8.90 (s, 1H), 8.59-8.61 (m, 2H), 7.86 (dd, J=9.2 Hz, 4.0 Hz, 1H), 7.65-7.67 (m, 1H), 6.64 (s, 1H), 2.54 (s, 3H), 2.09 (t, J=18.4 Hz, 3H).

### Preparation 504

### N-(2-(2-oxabicyclo[2.1.1]hexan-1-yl)-6-methylpyrimidin-4-yl)-6′-chloro-5-(methoxymethyl)-[2,3′-bipyridin]-4′-amine

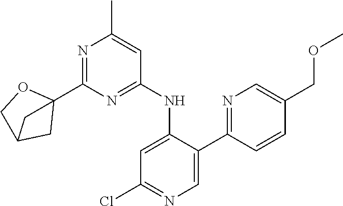 [SMILES: COCc1ccc(-c2cnc(Cl)cc2Nc2cc(C)nc(C34CC(CO3)C4)n2)nc1]

A mixture of 6′-chloro-5-(methoxymethyl)-[2,3′-bipyridin]-4′-amine (100 mg, 0.40 mmol), 2-(2-oxabicyclo[2.1.1]hexan-1-yl)-4-chloro-6-methylpyrimidine (Preparation 176, 126 mg, 0.60 mmol), Xantphos Pd G3 (38 mg, 40 mol) and NaOtBu (115 mg, 1.20 mmol) in dioxane (4 mL) was degassed with N₂, and heated to 90° C. for 30 min. The cooled mixture was filtered, the filtrate concentrated and the residue was purified by chromatography on silica gel (0-80% EtOAc-EtOH 3:1 with 20% NH₄OH in heptane) to give N-(2-(2-oxabicyclo[2.1.1]hexan-1-yl)-6-methylpyrimidin-4-yl)-6′-chloro-5-(methoxymethyl)-[2,3′-bipyridin]-4′-amine (88 mg, 51% yield) as an off-white solid. LCMS m/z=424 [M+H]⁺

### Preparation 505

### N-(2-chloro-5-iodopyridin-4-yl)-2-(1,1-difluoroethyl)pyrimidin-4-amine

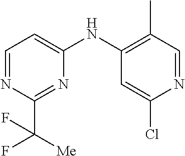 [SMILES: Cc1cnc(Cl)cc1Nc1ccnc(C(C)(F)F)n1]

To a solution of 2-chloro-5-iodopyridin-4-amine (500 mg, 1.96 mmol) in DMF (5 mL) was added 4-chloro-2-(1,1-difluoroethyl)pyrimidine (Preparation 148, 351 mg, 1.96 mmol), Pd₂(dba)₃ (180 mg, 0.197 mmol), Xantphos (113.7 mg, 0.197 mmol) and Cs₂CO₃ (1.28 g, 3.93 mmol) and the mixture stirred at 100° C. for 2 h under N₂. The reaction mixture was concentrated under reduced pressure and the residue diluted with water (30 mL) and extracted with EtOAc (30 mL×3). The combined organics were washed with brine (30 mL), dried (Na₂SO₄) and concentrated to give a residue which was purified on silica gel column chromatography (0-25% EtOAc/PE) to give N-(2-chloro-5-iodopyridin-4-yl)-2-(1,1-difluoroethyl)pyrimidin-4-amine as a yellow solid (480 mg, 61%). ¹H NMR (400 MHz, MeOH-d₄) δ ppm 8.65 (s, 1H), 8.56 (d, J=6.0 Hz, 1H), 8.47 (s, 1H), 7.24 (d, J=6.0 Hz, 1H), 2.00 (t, J=18.8 Hz, 3H).

### Preparation 506

### N-(2-chloro-5-(5-methyl-4,5,6,7-tetrahydropyrazolo[1,5-a]pyrazin-2-yl)pyridin-4-yl)-2-(1,1-difluoroethyl)pyrimidin-4-amine

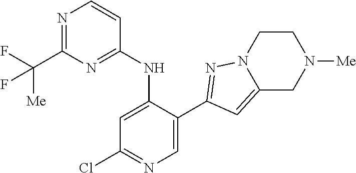 [SMILES: CN1CCn2nc(-c3cnc(Cl)cc3Nc3ccnc(C(C)(F)F)n3)cc2C1]

To a solution of N-(2-chloro-5-iodopyridin-4-yl)-2-(1,1-difluoroethyl)pyrimidin-4-amine (Preparation 505, 350 mg, 0.88 mmol) in toluene (3 mL), water (0.6 mL) and MeOH (3 mL) was added 2-bromo-5-methyl-4,5,6,7-tetrahydropyrazolo[1,5-a]pyrazine (Preparation 331, 286 mg, 1.32 mmol), (BPin)₂ (448 mg, 1.77 mmol), Pd(OAc)₂ (39.63 mg, 0.18 mmol), bis(1-adamantyl)-butyl-phosphane (127 mg, 0.35 mmol), CsF (536 mg, 3.53 mmol) and the mixture stirred at 90° C. for 2 h under N₂. The reaction mixture was evaporated to dryness and the residue diluted with water (15 mL) and extracted with EtOAc (20 mL×3). The combined organics were washed with brine (20 mL), dried (Na₂SO₄) and concentrated to give a residue which was purified on silica gel column chromatography (0-5% MeOH/DCM) to give N-(2-chloro-5-(5-methyl-4,5,6,7-tetrahydropyrazolo[1,5-a]pyrazin-2-yl)pyridin-4-yl)-2-(1,1-difluoroethyl)pyrimidin-4-amine as a yellow solid (80 mg, 22%). ¹H NMR (400 MHz, MeOH-d₄) δ ppm 9.04 (s, 1H), 8.60 (s, 1H), 8.53 (d, J=6.0 Hz, 1H), 7.07 (d, J=6.0 Hz, 1H), 6.66 (s, 1H), 4.35 (t, J=5.6 Hz, 2H), 3.77 (s, 2H), 3.04 (t, J=5.6 Hz, 2H), 2.01-2.10 (m, 6H).

### Preparation 507 to 508

The title compounds were prepared using N-(2-chloro-5-iodopyridin-4-yl)-2-(1,1-difluoroethyl)pyrimidin-4-amine (Preparation 505) and the appropriate halo-hetereocycle using an analogous method to that described for Preparation 506.

| Preparation | Name, Structure, Amine, Data |
|---|---|
| 507 | N-(2-chloro-5-(5-methyl-4,5,6,7-tetrahydrothiazolo[5,4-c]pyridin-2- yl)pyridin-4-yl)-2-(1,1-difluoroethyl)pyrimidin-4-amine 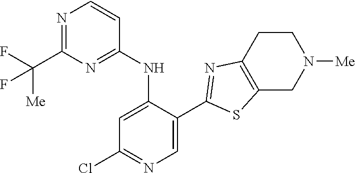 [SMILES: CN1CCc2nc(-c3cnc(Cl)cc3Nc3ccnc(C(C)(F)F)n3)sc2C1] SM: 2-bromo-5-methyl-4,5,6,7-tetrahydrothiazolo[5,4-c]pyridine ¹H NMR (400 MHz, CDCl₃) δ ppm 12.67 (s, 1H), 9.07 (s, 1H), 8.67 (s, 1H), 8.57 (d, J = 5.6 Hz, 1H), 6.80 (d, J = 6.0 Hz, 1H), 3.75 (s, 2H), 3.01-3.03 (m, 2H), 2.89-2.92 (m, 2H), 2.58 (s, 3H), 2.13-2.15 (m, 3H). |
| 508 | N-(2-chloro-5-(5-methyl-4,5,6,7-tetrahydrothiazolo[4,5-c]pyridin-2- yl)pyridin-4-yl)-2-(1,1-difluoroethyl)pyrimidin-4-amine 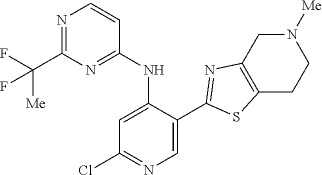 [SMILES: CN1CCc2sc(-c3cnc(Cl)cc3Nc3ccnc(C(C)(F)F)n3)nc2C1] SM: 2-bromo-5-methyl-4,5,6,7-tetrahydrothiazolo[4,5-c]pyridine ¹H NMR (400 MHz, MeOH-d₄) δ ppm: 9.12 (s, 1H), 8.70 (s, 1H), 8.58 (d, J = 6.0 Hz, 1H), 7.10 (d, J = 5.6 Hz, 1H), 3.84 (s, 2H), 3.03-3.05 (m, 2H), 2.93- 2.94 (m, 2H), 2.01-2.11 (m, 6H). |

### Preparation 509

### N-(4-((2-(1,1-difluoroethyl)-6-methylpyrimidin-4-yl)amino)-5-(5-formyl-1l-methyl-1H-pyrazol-3-yl)pyridin-2-yl)acetamide

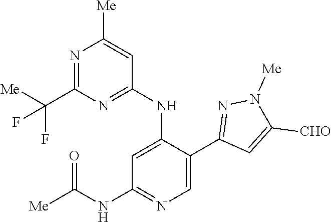 [SMILES: CC(=O)Nc1cc(Nc2cc(C)nc(C(C)(F)F)n2)c(-c2cc(C=O)n(C)n2)cn1]

To a solution of N-(5-bromo-4-((2-(1,1-difluoroethyl)-6-methylpyrimidin-4-yl)amino)pyridin-2-yl)acetamide (Preparation 483, 410 mg, 1.06 mmol) in H₂O (1 mL), toluene (5 mL) and MeOH (5 mL) was added 3-bromo-1-methyl-1H-pyrazole-5-carbaldehyde (301 mg, 1.59 mmol), (BPin)₂ (539 mg, 2.12 mmol), CsF (968 mg, 6.37 mmol), di(adamantan-1-yl)(butyl)phosphane (152 mg, 0.425 mmol) and Pd(OAc)₂ (47.7 mg, 0.212 mmol) and the resulting mixture stirred at 80° C. for 2 h under N₂. The mixture was concentrated and the residue purified by prep-HPLC (Method R, Gradient 31-61%) to give
N-(4-((2-(1,1-difluoroethyl)-6-methylpyrimidin-4-yl)amino)-5-(5-formyl-1-methyl-1H-pyrazol-3-yl)pyridin-2-yl)acetamide as a white solid (60 mg, 13.6%). LCMS m/z=416.2 [M+H]⁺.

### Preparation 510

### tert-butyl 2-(6-acetamido-4-((2-(1,1-difluoroethyl)-6-methylpyrimidin-4-yl)amino)pyridin-3-yl)-6,7-dihydrothiazolo[5,4-c]pyridine-5(4H)-carboxylate

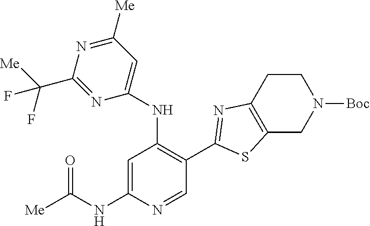 [SMILES: CC(=O)Nc1cc(Nc2cc(C)nc(C(C)(F)F)n2)c(-c2nc3c(s2)CN(C(=O)OC(C)(C)C)CC3)cn1]

To a solution of N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 300 mg, 0.854 mmol) in dioxane (5.0 mL) and H₂O (1.0 mL) was added tert-butyl 2-bromo-6,7-dihydrothiazolo[5,4-c]pyridine-5(4H)-carboxylate (327.3 mg, 1.03 mmol), Pd(dppf)Cl₂·DCM (69.8 mg, 85.4 mol), K₂CO₃ (236.2 mg, 1.71 mmol) and the mixture was stirred at 90° C. for 2 hours under N₂. The mixture was concentrated and purified by prep-HPLC (Method R, Gradient 57-87%) to give tert-butyl 2-(6-acetamido-4-((2-(1,1-difluoroethyl)-6-methylpyrimidin-4-yl)amino)pyridin-3-yl)-6,7-dihydrothiazolo[5,4-c]pyridine-5(4H)-carboxylate (60.0 mg, 12.9% yield) as a white solid. ¹H NMR (400 MHz, CDCl₃) δ ppm: 12.31 (s, 1H), 9.53 (br s, 1H), 8.55 (s, 1H), 8.09 (br s, 1H), 6.69 (s, 1H), 4.70 (s, 2H), 3.82-3.83 (m, 2H), 3.00-3.01 (m, 2H), 2.56 (s, 3H), 2.16-2.26 (m, 6H), 1.51 (s, 9H).

### Preparation 511

### N-(4-acetyl-4′-((2-(1,1-difluoroethyl)-6-methylpyrimidin-4-yl)amino)-[2,3′-bipyridin]-6′-yl)acetamide

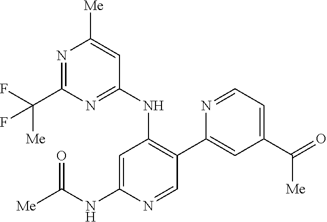 [SMILES: CC(=O)Nc1cc(Nc2cc(C)nc(C(C)(F)F)n2)c(-c2cc(C(C)=O)ccn2)cn1]

N-(4-acetyl-4′-((2-(1,1-difluoroethyl)-6-methylpyrimidin-4-yl)amino)-[2,3′-bipyridin]-6′-yl)acetamide was obtained (210 mg, 34%) from 1-(2-chloropyridin-4-yl)ethan-1-one and N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486), following a similar procedure to that described in Preparation 510. LCMS m/z=427.2 [M+H]⁺

### Preparation 512

### N-(4-((2-(1,1-difluoroethyl)-6-methylpyrimidin-4-yl)amino)-5-(5-formylthiazol-2-yl)pyridin-2-yl)acetamide

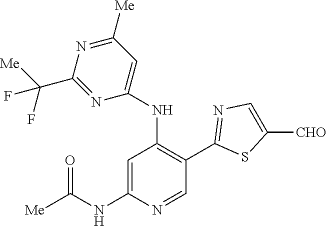 [SMILES: CC(=O)Nc1cc(Nc2cc(C)nc(C(C)(F)F)n2)c(-c2ncc(C=O)s2)cn1]

To a solution of (6-acetamido-4-((2-(1,1-difluoroethyl)-6-methylpyrimidin-4-yl)amino)pyridin-3-yl)boronic acid (Preparation 488, 1.5 g, 4.27 mmol) in dioxane (20 mL) and H₂O (4.0 mL) was added 2-bromothiazole-5-carbaldehyde (273.5 mg, 1.42 mmol), Pd(dppf)Cl₂·DCM (116.3 mg, 0.142 mmol) and K₂CO₃ (590 mg, 4.27 mmol) and the mixture stirred at 90° C. for 2 h under N₂. The mixture was diluted with water (15 mL) and extracted with EtOAc (20 mL×3). The combined organics were washed with brine (60 mL), dried (Na₂SO₄), concentrated and the residue purified by column chromatography (0-100% EtOAc/PE) to give N-(4-((2-(1,1-difluoroethyl)-6-methylpyrimidin-4-yl)amino)-5-(5-formylthiazol-2-yl)pyridin-2-yl)acetamide as a yellow solid (70 mg, crude) which was used without further purification. LCMS m/z=419.1 [M+H]⁺; ¹H NMR (500 MHz, MeOH-d₄) δ ppm: 10.08 (s, 1H), 9.48 (s, 1H), 8.83 (s, 1H), 8.67 (s, 1H), 7.05 (s, 1H), 2.53 (s, 3H), 2.21 (s, 3H), 2.14 (t, J=18.5 Hz, 3H).

### Preparation 513

### tert-butyl 2-(6-acetamido-4-((2-(1,1-difluoroethyl)pyrimidin-4-yl)amino)pyridin-3-yl)-6,7-dihydropyrazolo[1,5-a]pyrazine-5(4H)-carboxylate

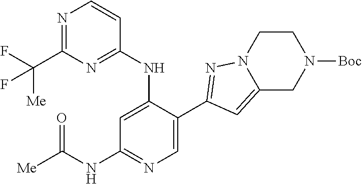 [SMILES: CC(=O)Nc1cc(Nc2ccnc(C(C)(F)F)n2)c(-c2cc3n(n2)CCN(C(=O)OC(C)(C)C)C3)cn1]

tert-butyl 2-(6-acetamido-4-((2-(1,1-difluoroethyl)pyrimidin-4-yl)amino)pyridin-3-yl)-6,7-dihydropyrazolo[1,5-a]pyrazine-5(4H)-carboxylate was obtained as a yellow solid, 160 mg, 69.9%, from (6-acetamido-4-((2-(1,1-difluoroethyl)pyrimidin-4-yl)amino)pyridin-3-yl)boronic acid (Preparation 487) and tert-butyl 2-bromo-6,7-dihydropyrazolo[1,5-a]pyrazine-5(4H)-carboxylate following a similar procedure to that described in Preparation 512. ¹H NMR (500 MHz, CDCl₃) δ ppm 11.43 (s, 1H), 9.38 (s, 1H), 8.54 (d, J=4.4 Hz, 1H), 8.46 (s, 1H), 8.32 (s, 1H), 6.91 (s, 1H), 6.46 (s, 1H), 4.67-4.74 (m, 2H), 4.20-4.28 (m, 2H), 3.90-3.98 (m, 2H), 2.14-2.25 (m, 6H), 1.52 (s, 9H)

### Preparation 514

### tert-butyl 2-(6-acetamido-4-((2-(1,1-difluoroethyl)-6-methylpyrimidin-4-yl)amino)pyridin-3-yl)-6,7-dihydropyrazolo[1,5-a]pyrazine-5(4H)-carboxylate

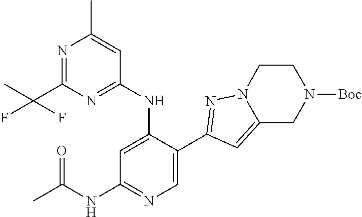 [SMILES: CC(=O)Nc1cc(Nc2cc(C)nc(C(C)(F)F)n2)c(-c2cc3n(n2)CCN(C(=O)OC(C)(C)C)C3)cn1]

A vial containing tert-butyl tert-butyl 2-bromo-6,7-dihydropyrazolo[1,5-a]pyrazine-5(4H)-carboxylate (201 mg, 0.665 mmol), N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 650 mg, 1.50 mmol), PCy₃-Pd-G3 (96 mg, 0.130 mmol) and KOAc (1.5 M, 2.40 mmol, 1.6 mL) in dioxane (6 mL) was degassed then backfilled with N₂ then heated to 75° C. for 9 h. The mixture was cooled to rt then loaded onto a silica gel column and purified with (20-85% EtOAc with 2% dimethylethylamine in heptane then 100% 3:1 EtOAc) to afford tert-butyl 2-(6-acetamido-4-((2-(1,1-difluoroethyl)-6-methylpyrimidin-4-yl)amino)pyridin-3-yl)-6,7-dihydropyrazolo[1,5-a]pyrazine-5(4H)-carboxylate (350 mg, crude) as a white solid. LCMS m/z=529.2 [M+H]⁺.

### Preparation 515

### tert-butyl 2-(6-acetamido-4-((2-(1,1-difluoroethyl)pyrimidin-4-yl)amino)pyridin-3-yl)-4,6-dihydro-5H-pyrrolo[3,4-d]thiazole-5-carboxylate

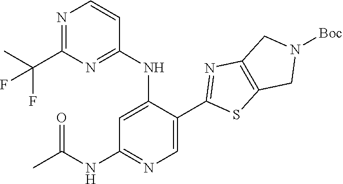 [SMILES: CC(=O)Nc1cc(Nc2ccnc(C(C)(F)F)n2)c(-c2nc3c(s2)CN(C(=O)OC(C)(C)C)C3)cn1]

tert-butyl 2-(6-acetamido-4-((2-(1,1-difluoroethyl)pyrimidin-4-yl)amino)pyridin-3-yl)-4,6-dihydro-5H-pyrrolo[3,4-d]thiazole-5-carboxylate was obtained from N-(4-((2-(1,1-difluoroethyl)pyrimidin-4-yl)amino)-5-(4,4,5,5-tetramethyl-1,3,2-dioxaborolan-2-yl)pyridin-2-yl)acetamide (Preparation 485) and tert-butyl 2-bromo-4,6-dihydro-5H-pyrrolo[3,4-d]thiazole-5-carboxylate, following the procedure described in Preparation 514.

### Preparation 516

### tert-butyl 2-(6-acetamido-4-((2-(1,1-difluoroethyl)-6-ethylpyrimidin-4-yl)amino)pyridin-3-yl)-6,7-dihydropyrazolo[1,5-a]pyrazine-5(4H)-carboxylate

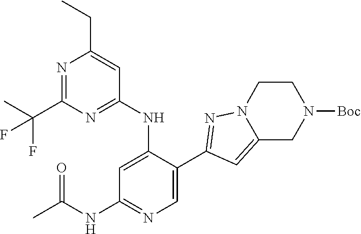 [SMILES: CCc1cc(Nc2cc(NC(C)=O)ncc2-c2cc3n(n2)CCN(C(=O)OC(C)(C)C)C3)nc(C(C)(F)F)n1]

To suspension of N-(4-((2-(1,1-difluoroethyl)-6-ethylpyrimidin-4-yl)amino)-5-(4,4,5,5-tetramethyl-1,3,2-dioxaborolan-2-yl)pyridin-2-yl)acetamide (Preparation 491, 200 mg, 0.447 mmol) in 2-Me-THF (2 mL) and water (0.4 mL) were added tert-butyl 2-bromo-6,7-dihydro-4H-pyrazolo[1,5-a]pyrazine-5-carboxylate (202.7 mg, 0.671 mmol) and K₃PO₄ (284.7 mg, 1.34 mmol). The resulting mixture was sparged with N₂ for 10 min. SPhos Pd G3 (34.9 mg, 44.7 mol) was added and the reaction was stirred at 80° C. for 16 h under N₂. The cooled mixture was filtered, washing through with water and EtOAc and the filtrate separated. The aqueous phase was extracted with EtOAc (2) and the combined organic extracts washed with brine, dried (MgSO₄) and concentrated in vacuo. The residue was purified by silica gel chromatography (0-100% EtOAc in Hept) to give tert-butyl 2-(6-acetamido-4-((2-(1,1-difluoroethyl)-6-ethylpyrimidin-4-yl)amino)pyridin-3-yl)-6,7-dihydropyrazolo[1,5-a]pyrazine-5(4H)-carboxylate (170 mg) as an off-white solid. LCMS [M+1]=543.5

### Preparation 517

### N-(5-(5-acetylpyrazin-2-yl)-4-((2-(1,1-difluoroethyl)-6-methylpyrimidin-4-yl)amino)pyridin-2-yl)acetamide

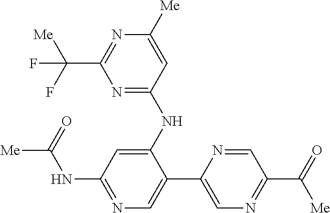 [SMILES: CC(=O)Nc1cc(Nc2cc(C)nc(C(C)(F)F)n2)c(-c2cnc(C(C)=O)cn2)cn1]

To a solution of N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 500 mg, 1.15 mmol) in 2-MeTHF (10 mL) and H₂O (2 mL) was added 1-(5-chloropyrazin-2-yl)ethan-1-one (361 mg, 2.31 mmol), XPhos Pd G3 (97.7 mg, 0.115 mmol) and K₃PO₄ (735 mg, 3.46 mmol) and the mixture stirred at 80° C. for 1 h under N₂. The mixture was concentrated under reduced pressure and the residue purified by column chromatography (100% EtOAc followed by 16% MeOH/DCM) to give N-(5-(5-acetylpyrazin-2-yl)-4-((2-(1,1-difluoroethyl)-6-methylpyrimidin-4-yl)amino)pyridin-2-yl)acetamide as a yellow solid (286 mg, 58%).

LCMS m/z=428.2 [M+H]⁺.

### Preparation 518

### N-(4-((2-(1,1-difluoroethyl)-6-ethylpyrimidin-4-yl)amino)-5-(4,5,6,7-tetrahydropyrazolo[1,5-a]pyrazin-2-yl)pyridin-2-yl)acetamide trifluoroacetate

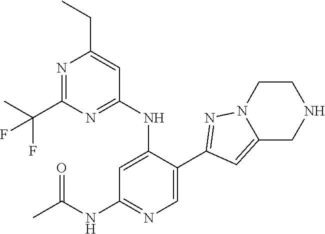 [SMILES: CCc1cc(Nc2cc(NC(C)=O)ncc2-c2cc3n(n2)CCNC3)nc(C(C)(F)F)n1]

To tert-butyl 2-(6-acetamido-4-((2-(1,1-difluoroethyl)-6-ethylpyrimidin-4-yl)amino)pyridin-3-yl)-6,7-dihydropyrazolo[1,5-a]pyrazine-5(4H)-carboxylate (Preparation 516, 170 mg, 0.313 mmol) in DCM (2 mL) was added TFA (369.88 mg, 3.24 mmol) at 23° C. The reaction was stirred at 23° C. for 16 h, then evaporated under reduced pressure to give N-(4-((2-(1,1-difluoroethyl)-6-ethylpyrimidin-4-yl)amino)-5-(4,5,6,7-tetrahydropyrazolo[1,5-a]pyrazin-2-yl)pyridin-2-yl)acetamide trifluoroacetate. LCMS m/z=443.4 [M+H]⁺

### Preparation 519

### N-(4-((2-(1,1-difluoroethyl)-6-methylpyrimidin-4-yl)amino)-5-(4,5,6,7-tetrahydrothiazolo[5,4-c]pyridin-2-yl)pyridin-2-yl)acetamide

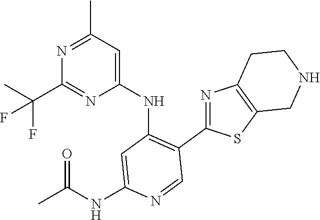 [SMILES: CC(=O)Nc1cc(Nc2cc(C)nc(C(C)(F)F)n2)c(-c2nc3c(s2)CNCC3)cn1]

To a solution of tert-butyl 2-(6-acetamido-4-((2-(1,1-difluoroethyl)-6-methylpyrimidin-4-yl)amino)pyridin-3-yl)-6,7-dihydrothiazolo[5,4-c]pyridine-5(4H)-carboxylate (Preparation 510, 55 mg, 0.10 mmol) in DCM (2 mL) was added HCl/dioxane (4 M, 0.5 mL). The reaction was concentrated under reduced pressure to give N-(4-((2-(1,1-difluoroethyl)-6-methylpyrimidin-4-yl)amino)-5-(4,5,6,7-tetrahydrothiazolo[5,4-c]pyridin-2-yl)pyridin-2-yl)acetamide hydrochloride as a yellow solid (50 mg, crude) which was used without purification. ¹H NMR (400 MHz, MeOH-d₄) δ ppm: 8.95 (s, 1H), 8.73 (s, 1H), 7.18 (s, 1H), 3.72 (t, J=6.0 Hz, 2H), 3.66 (s, 2H), 3.38 (t, J=5.6 Hz, 2H), 2.60 (s, 3H), 2.33 (s, 3H), 2.11 (t, J=18.4 Hz, 3H).

### Preparation 520

### N-(5-bromo-2-chloropyridin-4-yl)-6-chloro-2-(1,1-difluoroethyl)pyrimidin-4-amine

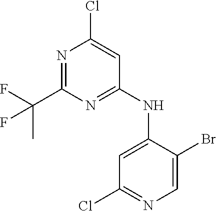 [SMILES: CC(F)(F)c1nc(Cl)cc(Nc2cc(Cl)ncc2Br)n1]

A mixture of 5-bromo-2-chloro-pyridin-4-amine (250 mg, 1.21 mmol), Cs₂CO₃ (1 g, 3.07 mmol) and DMF (5 mL) was stirred for 5 mins, then 4,6-dichloro-2-(1,1-difluoroethyl)pyrimidine (300 mg, 1.41 mmol) was added and the reaction mixture stirred at rt for 1 h. The reaction was warmed to 35° C. for 2 h, then MeOH (3 mL) added. The reaction was warmed to 50° C. and stirred overnight. The cooled reaction was concentrated in vacuo, the residue diluted with water (10 mL) and extracted with EtOAc (3×10 mL). The combined organic layers were concentrated to dryness and purified via chromatography (heptane to 50% EtOAc) to give N-(5-bromo-2-chloro-4-pyridyl)-6-chloro-2-(1,1-difluoroethyl)pyrimidin-4-amine (415 mg, 89.7% yield). LCMS m/z=383.1 [M+H]+.

### Preparation 521

### N-(5-bromo-2-chloropyridin-4-yl)-2-(1,1-difluoroethyl)-6-methoxypyrimidin-4-amine

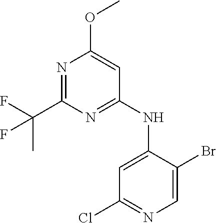 [SMILES: COc1cc(Nc2cc(Cl)ncc2Br)nc(C(C)(F)F)n1]

N-(5-Bromo-2-chloro-4-pyridyl)-6-chloro-2-(1,1-difluoroethyl)pyrimidin-4-amine was treated with MeOH (10 mL) and K₂CO₃ (400 mg), then heated to 70° C. and the reaction was stirred overnight. The cooled reaction was concentrated in vacuo, diluted with water (10 mL) and extracted with EtOAc (3×10 mL). The combined organic layers were concentrated to dryness and purified via silica gel chromatography (heptane to 50% EtOAc) to give N-(5-bromo-2-chloropyridin-4-yl)-2-(1,1-difluoroethyl)-6-methoxypyrimidin-4-amine (390 mg, 85.3% yield). LCMS m/z=379.1 [M+H]+.

### Preparation 522

### N-(2-chloro-5-(1-methyl-1H-pyrazol-3-yl)pyridin-4-yl)-2-(1,1-difluoroethyl)-6-methoxypyrimidin-4-amine

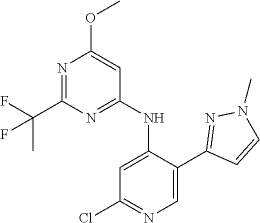 [SMILES: COc1cc(Nc2cc(Cl)ncc2-c2ccn(C)n2)nc(C(C)(F)F)n1]

A mixture of N-(5-bromo-2-chloropyridin-4-yl)-2-(1,1-difluoroethyl)-6-methoxypyrimidin-4-amine (Preparation 521, 150 mg, 0.395 mmol), 1-methyl-3-(4,4,5,5-tetramethyl-1,3,2-dioxaborolan-2-yl)pyrazole (100 mg, 0.481 mmol), K₂CO₃ (150 mg, 1.09 mmol) and Pd(dppf)Cl₂ (15 mg, 20.5 umol) in dioxane (3 mL) and water (1 mL) was sealed and warmed to 80° C. for 20 h. The cooled reaction was diluted with water (10 mL) and extracted with EtOAc (3×10 mL). The combined organic layers were concentrated to dryness, then purified via silica gel chromatography (heptane to EtOAc) to give N-(2-chloro-5-(1-methyl-1H-pyrazol-3-yl)pyridin-4-yl)-2-(1,1-difluoroethyl)-6-methoxypyrimidin-4-amine as a white solid, (30 mg, 19.9% yield). LCMS m/z=381.1 [M+H]⁺.

### Example 1

### N-(4-((6-(1,1-difluoroethyl)pyridin-2-yl)amino)-5-ethoxypyridin-2-yl)acetamide

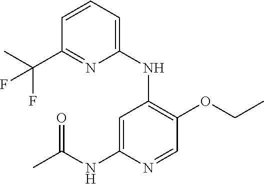 [SMILES: CCOc1cnc(NC(C)=O)cc1Nc1cccc(C(C)(F)F)n1]

To a solution of N-(4-amino-5-ethoxypyridin-2-yl)acetamide hydrochloride (Preparation 62, 100 mg, 0.432 mmol) in DMF (5 mL) was added 2-bromo-6-(1,1-difluoroethyl)pyridine (144 mg, 0.647 mmol), BrettPhos Pd G3 (39.1 mg, 0.043 mmol) and Cs₂CO₃ (422 mg, 1.29 mmol) and the reaction was stirred at 100° C. for 1.5 h under N₂. The mixture was concentrated and purified by prep-HPLC (Method B, Gradient 23-53%) to give N-(4-((6-(1,1-difluoroethyl)pyridin-2-yl)amino)-5-ethoxypyridin-2-yl)acetamide (31.0 mg, 21.4% yield) as a white solid. LCMS m/z=337.4 [M+H]⁺. ¹H NMR: (500 MHz, DMSO-d₆) δ ppm: 10.09 (s, 1H), 9.09 (s, 1H), 8.59 (s, 1H), 7.91 (s, 1H), 7.80 (t, J=8.0 Hz, 1H), 7.38 (d, J=8.0 Hz, 1H), 7.18 (d, J=7.5 Hz, 1H), 4.20-4.15 (m, 2H), 2.14-2.03 (m, 6H), 1.40 (t, J=7.0 Hz, 3H).

### Examples 2 to 5

The compounds in the following table were prepared from N-(4-amino-5-ethoxypyridin-2-yl)acetamide hydrochloride (Preparation 62) and the appropriate substituted pyridine or pyrimidine (Het) following a similar procedure to that described in Example 1.

| Example | |
|---|---|
| No | Name/Structure/Het/Data |
| 2 | N-(4-((4-(1,1-difluoroethyl)pyridin-2-yl)amino)-5-ethoxypyridin-2- yl)acetamide 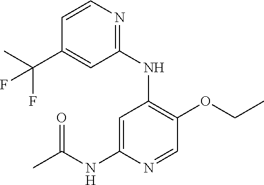 [SMILES: CCOc1cnc(NC(C)=O)cc1Nc1cc(C(C)(F)F)ccn1] Het: 2-chloro-4-(1,1-difluoroethyl)pyridine Prep-HPLC (Method C, Gradient 35-65%) 28 mg, 20.3% yield as a yellow solid. LCMS m/z = 337.1 [M + H]⁺. ¹H NMR: (400 MHz, CDCl₃) δ ppm: 9.00 (s, 1H), 8.45 (d, J = 5.2 Hz, 1H), 7.76 (s, 2H), 7.40 (s, 1H), 7.15 (s, 1H), 6.99 (d, J = 4.8 Hz, 1H), 4.17 (q, J = 7.2 Hz, 2H), 2.18 (s, 3H), 1.95 (t, J = 18.4 Hz, 3H), 1.50 (t, J = 6.8 Hz, 3H). |
| 3 | N-(4-((4-(1,1-difluoroethyl)pyrimidin-2-yl)amino)-5-ethoxypyridin-2- yl)acetamide 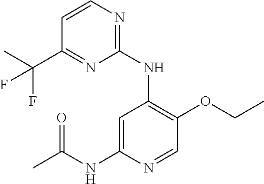 [SMILES: CCOc1cnc(NC(C)=O)cc1Nc1nccc(C(C)(F)F)n1] Het: 2-chloro-4-(1,1-difluoroethyl)pyrimidine Prep-HPLC (Method B, 35-65%) 23.0 mg, 15.8% yield as a white solid. LCMS m/z = 338.1 [M + H]⁺. ¹H NMR: (500 MHz, CDCl₃) δ ppm: 9.37 (s, 1H), 8.68 (d, J = 5.5 Hz, 1H), 8.06 (s, 1H), 7.79 (s, 2H), 7.16 (d, J = 5.0 Hz, 1H), 4.19 (q, J = 7.0 Hz, 2H), 2.10-2.19 (m, 6H), 1.51 (t, J = 7.0 Hz, 3H). |
| 4 | N-(4-((2-(1,1-difluoroethyl)pyrimidin-4-yl)amino)-5-ethoxypyridin-2- yl)acetamide 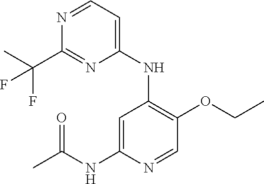 [SMILES: CCOc1cnc(NC(C)=O)cc1Nc1ccnc(C(C)(F)F)n1] Het: 4-chloro-2-(1,1-difluoroethyl)pyrimidine Prep-HPLC (Method B, Gradient 22-52%) 50.0 mg, 34.3% yield as a white solid. LCMS m/z = 338.0 [M + H]⁺. ¹H NMR: (500 MHz, DMSO-d₆) δ ppm: 10.22 (s, 1H), 9.27 (s, 1H), 8.98 (s, 1H), 8.50 (d, J = 6.0 Hz, 1H), 8.02 (s, 1H), 7.24-7.30 (m, 1H), 4.19 (q, J = 7.0 Hz, 2H), 2.10- 2.02 (m, 6H), 1.36 (t, J = 7.0 Hz, 3H). |
| 5 | N-(4-((2-(1,1-difluoroethyl)-6-methoxypyrimidin-4-yl)amino)-5- ethoxypyridin-2-yl)acetamide 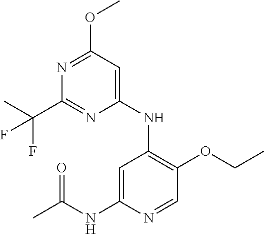 [SMILES: CCOc1cnc(NC(C)=O)cc1Nc1cc(OC)nc(C(C)(F)F)n1] Het: 4-chloro-2-(1,1-difluoroethyl)-6-methoxypyrimidine (Preparation 97)Prep-HPLC (Method B, Gradient 28 to 58%) 53.1 mg, 37.2% yield as a white solid. LCMS m/z = 368.1 [M + H]⁺. ¹H NMR: (500 MHz, CDCl₃) δ ppm: 8.75 (s, 1H), 7.88 (s, 1H), 7.80 (s, 1H), 7.50 (s, 1H), 6.48 (s, 1H), 4.17 (q, J = 7.0 Hz, 2H), 4.01 (s, 3H), 2.19 (s, 3H), 2.09 (t, J = 18.5 Hz, 3H), 1.49 (t, J = 7.0 Hz, 3H). |

### Examples 6 to 8

The compounds in the following table were prepared from the appropriate acetamide and 4-chloro-2-(1,1-difluoroethyl)pyrimidine, following a similar procedure to that described in Example 1.

| Example | |
|---|---|
| No | Name, Structure, Acetamide, Data, |
| 6 | N-(4-((2-(1,1-difluoroethyl)pyrimidin-4-yl)amino)-5- (difluoromethoxy)pyridin-2-yl)acetamide 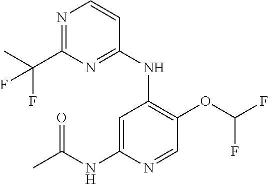 [SMILES: CC(=O)Nc1cc(Nc2ccnc(C(C)(F)F)n2)c(OC(F)F)cn1] Acetamide: N-(4-amino-5-(difluoromethoxy)pyridin-2-yl)acetamide hydrochloride (Preparation 65) Prep-HPLC (Method C, Gradient 36 to 56%) 10.2 mg, 8.8% yield as a yellow solid. LCMS m/z = 360.1 [M + H]⁺ ¹H NMR (400 MHz, CDCl₃) δ: ppm 9.12 (s, 1H), 8.62 (d, J = 6.0 Hz, 1H), 8.10 (s, 1H), 7.95 (s, 1H), 7.41 (s, 1H), 7.02 (d, J = 5.6 Hz, 1H), 6.58 (t, J = 72.4 Hz 1H), 2.23 (s, 3H), 2.15 (t, J = 18.8 Hz, 3H). |
| 7 | N-(5-cyclobutoxy-4-((2-(1,1-difluoroethyl)pyrimidin-4-yl)amino)pyridin-2- yl)acetamide 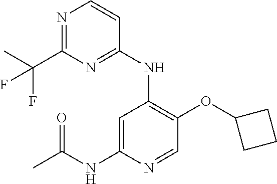 [SMILES: CC(=O)Nc1cc(Nc2ccnc(C(C)(F)F)n2)c(OC2CCC2)cn1] Acetamide: N-(4-amino-5-cyclobutoxypyridin-2-yl)acetamide hydrochloride (Preparation 68) Prep-HPLC (Method C, Gradient 37 to 57%) 5 mg, 5.5% yield as a white solid. LCMS m/z = 364.2 [M + H]⁺ ¹H NMR: (500 MHz, CDCl₃) δ ppm: 8.95 (s, 1H), 8.57 (d, J = 6.0 Hz, 1H), 7.93 (s, 1H), 7.69 (s, 1H), 7.59 (s, 1H), 7.04 (d, J = 6.0 Hz, 1H), 4.707-4.70 (m, 1H), 2.52-2.49 (m, 2H), 2.27-2.22 (m, 2H), 2.20-2.10 (m, 6H), 1.92-1.90 (m, 1H), 1.74-1.71 (m, 1H). |
| 8 | N-(4-((2-(1,1-difluoroethyl)pyrimidin-4-yl)amino)-5-isopropoxypyridin-2- yl)acetamide 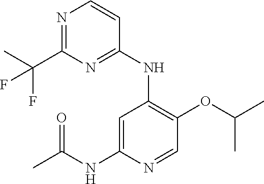 [SMILES: CC(=O)Nc1cc(Nc2ccnc(C(C)(F)F)n2)c(OC(C)C)cn1] Acetamide: N-(4-amino-5-isopropoxypyridin-2-yl)acetamide hydrochloride (Preparation 66) Prep-HPLC (Method G, Gradient: 33% to 63%) 9.4 mg, 7.0% yield as a yellow solid. LCMS m/z = 352.0 [M + H]⁺ 1H NMR: (500 MHz, CDCl3) δ: ppm 8.97 (s, 1H), 8.56 (d, J = 6.0 Hz, 1H), 7.85-7.82 (m, 2H), 7.60 (s, 1H), 7.02 (d, J = 5.5 Hz, 1H), 4.67-4.61 (m, 1H), 2.20-2.11 (m, 6H), 1.42 (d, J = 6.0 Hz, 6H). |

### Example 9

### N-(4-((2-(1,1-difluoroethyl)-6-methylpyrimidin-4-yl)amino)-5-ethoxypyridin-2-yl)propionamide

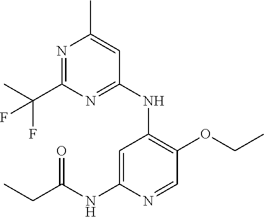 [SMILES: CCOc1cnc(NC(=O)CC)cc1Nc1cc(C)nc(C(C)(F)F)n1]

N-(4-((2-(1,1-Difluoroethyl)-6-methylpyrimidin-4-yl)amino)-5-ethoxypyridin-2-yl)propionamide

was obtained as a white solid, 28 mg, 16% yield from N-(4-amino-5-ethoxypyridin-2-yl)propionamide hydrochloride (Preparation 73) and 4-chloro-2-(1,1-difluoroethyl)-6-methylpyrimidine (Preparation 92) following a similar procedure to that described in Example 1. The crude was purified by prep-HPLC (Method B, Gradient 30-60%). LCMS m/z=366.1 [M+H]⁺ ¹H NMR: (500 MHz, CDCl₃) δ ppm: 8.96 (s, 1H), 7.81 (s, 1H), 7.78 (s, 1H), 7.51 (s, 1H), 6.90 (s, 1H), 4.18 (q, J=7.0 Hz, 2H), 2.57 (s, 3H), 2.41 (q, J=7.5 Hz, 2H), 2.14 (t, J=19.0 Hz, 3H), 1.50 (t, J=7.0 Hz, 3H), 1.26 (t, J=7.5 Hz, 3H).

### Example 10N-(4-((2-(1,1-difluoroethyl)-6-methylpyrimidin-4-yl)amino)-5-ethoxypyridin-2-yl)acetamide

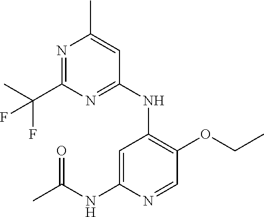 [SMILES: CCOc1cnc(NC(C)=O)cc1Nc1cc(C)nc(C(C)(F)F)n1]

To a solution of N-(4-amino-5-ethoxypyridin-2-yl)acetamide hydrochloride (Preparation 62, 1.5 g, 6.47 mmol) and 4-chloro-2-(1,1-difluoroethyl)-6-methylpyrimidine (Preparation 92, 1.3 g, 6.47 mmol) in dioxane (10.0 mL) was added Cs₂CO₃ (6.3 g, 19.42 mmol) and BrettPhos Pd G3 (587 mg, 0.647 mmol) at 25° C. The mixture was stirred at 100° C. for 2 h under N₂. The mixture was concentrated under reduced pressure and the residue was purified by prep-HPLC (Method A, Gradient 25 to 55%) to give N-(4-((2-(1,1-difluoroethyl)-6-methylpyrimidin-4-yl)amino)-5-ethoxypyridin-2-yl)acetamide (1.1 g, 49.7% yield) as a white solid. LCMS m/z=352.1 [M+H]⁺. ¹H NMR: (400 MHz, CDCl₃) δ ppm: 9.00 (s, 1H), 7.88 (br s, 1H), 7.80 (s, 1H), 7.53 (s, 1H), 6.88 (s, 1H), 4.18 (q, J=7.2 Hz, 2H), 2.55 (s, 3H), 2.21 (s, 3H), 2.15 (t, J=18.8 Hz, 3H), 1.50 (t, J=6.8 Hz, 3H).

### Example 11N-(5-(cyclopropylmethoxy)-4-((2-(1,1-difluoroethyl)-6-methylpyrimidin-4-yl)amino)pyridin-2-yl)acetamide

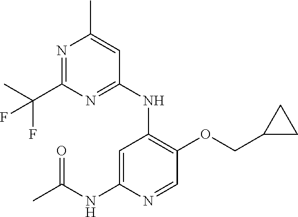 [SMILES: CC(=O)Nc1cc(Nc2cc(C)nc(C(C)(F)F)n2)c(OCC2CC2)cn1]

To a solution of N-(4-amino-5-(cyclopropylmethoxy)pyridin-2-yl)acetamide hydrochloride (Preparation 64, 70.0 mg, 0.272 mmol) in dioxane (5 mL) was added 4-chloro-2-(1,1-difluoroethyl)-6-methylpyrimidine (Preparation 92, 62.7 mg, 0.326 mmol), BrettPhos Pd G3 (24.6 mg, 0.0272 mmol) and Cs₂CO₃ (265 mg, 0.815 mmol). The mixture was stirred at 100° C. for 2 h under N₂. The mixture was concentrated and purified by prep-HPLC (Method C, Gradient 36 to 68%) to give N-(5-(cyclopropylmethoxy)-4-((2-(1,1-difluoroethyl)-6-methylpyrimidin-4-yl)amino)pyridin-2-yl)acetamide (19.0 mg, 18.5% yield) as a white solid. LCMS m/z=378.1 [M+H]⁺. ¹H NMR: (500 MHz, CDCl₃) δ ppm: 8.99 (s, 1H), 8.26 (s, 1H), 7.79 (s, 1H), 7.61 (s, 1H), 6.87 (s, 1H), 3.92 (d, J=6.5 Hz, 2H), 2.54 (s, 3H), 2.18-2.10 (m, 6H), 1.31-1.26 (m, 1H), 0.70-0.68 (m, 2H), 0.38-0.36 (m, 2H).

### Examples 12 to 19

The compounds in the following table were prepared from N-(4-amino-5-ethoxypyridin-2-yl)acetamide hydrochloride (Preparation 62) or N-(4-amino-5-methoxypyridin-2-yl)acetamide hydrochloride (Preparation 61) and the appropriate substituted pyridine or pyrimidine (Het) following a similar procedure to that described in Example 10.

| | |
|---|---|
| Example No | Name, Structure, Het, Data |

Acetamide: N-(4-amino-5-ethoxypyridin-2-yl)acetamide hydrochloride (Preparation 62)

| | |
|---|---|
| 12 | N-(4-((2-(1,1-difluoroethyl)-6-ethylpyrimidin-4-yl)amino)-5- ethoxypyridin-2-yl)acetamide 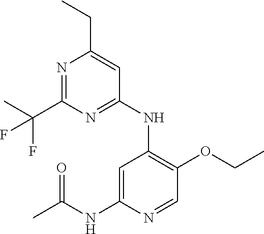 [SMILES: CCOc1cnc(NC(C)=O)cc1Nc1cc(CC)nc(C(C)(F)F)n1] Het: 4-chloro-2-(1,1-difluoroethyl)-6-ethylpyrimidine (Preparation 94)Prep-HPLC (Method B, Gradient: 20 to 50%) 18.0 mg, 13.7% yield as white solid. LCMS m/z = 366.1 [M + H]⁺. ¹H NMR: (400 MHz, CDCl₃) δ ppm: 8.98 (s, 1H), 8.23 (s, 1H), 7.80 (s, 1H), 7.56 (s, 1H), 6.89 (s, 1H), 4.17 (q, J = 7.2 Hz, 2H), 2.81 (q, J = 7.6 Hz, 2H), 2.09-2.19 (m, 6H), 1.49 (t, J = 6.8 Hz, 3H), 1.33 (t, J = 7.6 Hz, 3H). |
| 13 | N-(4-((6-cyclopropyl-2-(1,1-difluoroethyl)pyrimidin-4-yl)amino)-5- ethoxypyridin-2-yl)acetamide 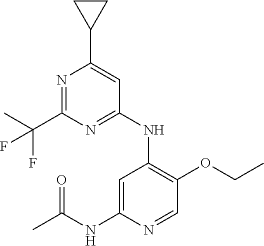 [SMILES: CCOc1cnc(NC(C)=O)cc1Nc1cc(C2CC2)nc(C(C)(F)F)n1] Het: 4-chloro-6-cyclopropyl-2-(1,1-difluoroethyl)pyrimidine (Preparation 96) Prep-HPLC (Method B, Gradient 33 to 63%) 33.2 mg, 24.5% yield as a white solid. LCMS m/z = 378.1 [M + H]⁺. ¹H NMR: (500 MHz, CDCl₃) δ ppm: 8.85 (s, 1H), 7.94 (s, 1H), 7.80 (s, 1H), 7.49 (s, 1H), 6.91 (s, 1H), 4.18 (q, J = 7.5 Hz, 2H), 2.19 (s, 3H), 2.11- 2.02 (m, 4H), 1.49 (t, J = 7.0 Hz, 3H), 1.20-1.18 (m, 2H), 1.08-1.06 (m, 2H). |
| 14 | N-(4-((6-(1,1-difluoroethyl)-4-((1r,3r)-3-methoxycyclobutoxy)pyridin-2- yl)amino)-5-ethoxypyridin-2-yl)acetamide 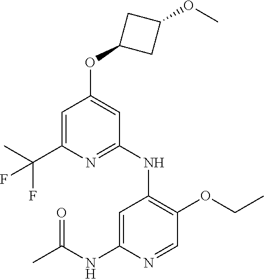 [SMILES: CCOc1cnc(NC(C)=O)cc1Nc1cc(O[C@H]2C[C@H](OC)C2)cc(C(C)(F)F)n1] Het: 2-chloro-6-(1,1-difluoroethyl)-4-((1r,3r)-3- methoxycyclobutoxy)pyridine (Preparation 88) Prep-HPLC (Method B, 35 to 65%) 27.0 mg, 24.15% yield as a pink solid. LCMS m/z = 437.2 [M + H]⁺. ¹H NMR: (500 MHz, CDCl₃) δ ppm: 8.90 (s, 1H), 8.13 (s, 1H), 7.73 (s, 1H), 7.30 (s, 1H), 6.74 (d, J = 2.0 Hz, 1H), 6.39 (s, 1H), 4.95-4.91 (m, 1H), 4.19-4.13 (m, 3H), 3.28 (s, 3H), 2.53-2.44 (m, 4H), 2.16-2.06 (m, 6H), 1.49 (t, J = 7.0 Hz, 3H). |
| 15 | N-(4-((6-(1,1-difluoroethyl)-4-((1s,3s)-3-methoxycyclobutoxy)pyridin-2- yl)amino)-5-ethoxypyridin-2-yl)acetamide 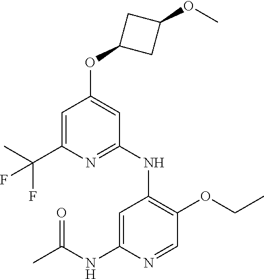 [SMILES: CCOc1cnc(NC(C)=O)cc1Nc1cc(O[C@H]2C[C@@H](OC)C2)cc(C(C)(F)F)n1] Het: 2-chloro-6-(1,1-difluoroethyl)-4-((1s,3s)-3- methoxycyclobutoxy)pyridine (Preparation 89) Prep-HPLC (Method C, 46 to 76%) 29.0 mg, 34.3% yield as a white solid. LCMS m/z = 437.2 [M + H]⁺. ¹H NMR: (500 MHz, DMSO-d₆) δ ppm: 10.03 (s, 1H), 9.04 (s, 1H), 8.44 (s, 1H), 7.86 (s, 1H), 6.83 (s, 1H), 6.62 (s, 1H), 4.44-4.40 (m, 1H), 4.15 (q, J = 7.0 Hz 2H), 3.61-3.59 (m, 1H), 3.12 (s, 3H), 2.89-2.85 (m, 2H), 2.07- 1.98 (m, 6H), 2.00-1.87 (m, 2H), 1.36 (t, J = 7.0 Hz, 3H). |
| 16 | N-(4-((2-(1,1-difluoroethyl)-6-((1r,3r)-3-methoxycyclobutoxy)pyrimidin- 4-yl)amino)-5-ethoxypyridin-2-yl)acetamide 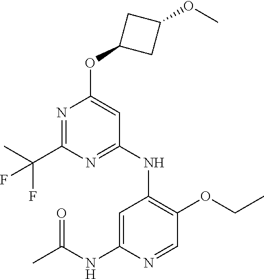 [SMILES: CCOc1cnc(NC(C)=O)cc1Nc1cc(O[C@H]2C[C@H](OC)C2)nc(C(C)(F)F)n1] Het: 4-chloro-2-(1,1-difluoroethyl)-6-((1r,3r)-3- methoxycyclobutoxy)pyrimidine (Preparation 111)Prep-HPLC (Method C, Gradient 33 to 63%) 48.1 mg, 30.2% yield as a white solid. LCMS m/z = 438.1 [M + H]⁺. ¹H NMR: (400 MHz, CDCl₃) δ ppm: 8.72 (s, 1H), 8.14 (s, 1H), 7.80 (s, 1H), 7.53 (s, 1H), 6.41 (s, 1H), 5.35-5.40 (m, 1H), 4.14-4.20 (m, 2H), 4.09- 4.13 (m, 1H), 3.27 (s, 3H), 2.48-2.52 (m, 2H), 2.43-2.45 (m, 2H), 2.19 (s, 3H), 2.05 (t, J = 18.8 Hz, 3H), 1.49 (t, J = 6.8 Hz, 3H). |

Acetamide: N-(4-amino-5-methoxypyridin-2-yl)acetamide hydrochloride (Preparation 61)

| | |
|---|---|
| 17 | N-(4-((6-cyclopropyl-2-(1,1-difluoroethyl)pyrimidin-4-yl)amino)-5- methoxypyridin-2-yl)acetamide 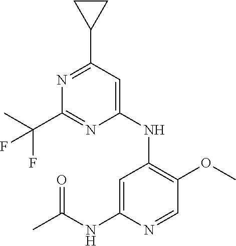 [SMILES: COc1cnc(NC(C)=O)cc1Nc1cc(C2CC2)nc(C(C)(F)F)n1] Het: 4-chloro-6-cyclopropyl-2-(1,1-difluoroethyl)pyrimidine (Preparation 96) Prep-HPLC (Method B, Gradient 27 to 57%) 13.8 mg, 11.5% yield as a white solid. LCMS m/z = 364.1 [M + H]⁺. ¹H NMR: (500 MHz, CDCl₃) δ ppm: 8.89 (s, 1H), 8.20 (s, 1H), 7.80(s, 1H), 7.52 (s, 1H), 6.89 (s, 1H), 3.95 (s, 3H), 2.19 (s, 3H), 2.11-2.03 (m, 4H), 1.19-1.07 (m, 4H). |
| 18 | N-(4-((2-(1,1-difluoroethyl)-6-methoxypyrimidin-4-yl)amino)-5- methoxypyridin-2-yl)acetamide 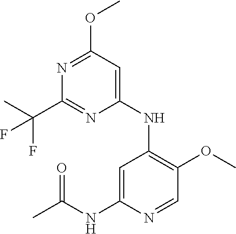 [SMILES: COc1cc(Nc2cc(NC(C)=O)ncc2OC)nc(C(C)(F)F)n1] Het: 4-chloro-2-(1,1-difluoroethyl)-6-methoxypyrimidine (Preparation 97) Prep-HPLC (Method H, Gradient: 15% to 35%) 10 mg, 5.13% yield as a white solid. LCMS m/z = 354.1 [M + H]⁺. ¹H NMR: (500 MHz, MeOH-d₄) δ ppm: 8.56 (s, 1H), 7.82 (s, 1H), 6.81 (s, 1H), 4.09-4.06 (m, 6H), 2.32 (s, 3H), 2.09 (t, J = 18.5 Hz, 3H). |
| 19 | N-(4-((2-(1,1-difluoroethyl)-6-((1r,3r)-3-methoxycyclobutoxy)pyrimidin- 4-yl)amino)-5-methoxypyridin-2-yl)acetamide 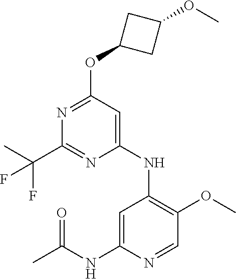 [SMILES: COc1cnc(NC(C)=O)cc1Nc1cc(O[C@H]2C[C@H](OC)C2)nc(C(C)(F)F)n1] Het: 4-chloro-2-(1,1-difluoroethyl)-6-((1r,3r)-3- methoxycyclobutoxy)pyrimidine (Preparation 111) Prep-HPLC (Method C, Gradient 30 to 60%) 52.8 mg, 34.7% yield as a white solid. LCMS m/z = 424.1 [M + H]⁺. ¹H NMR: (400 MHz, CDCl₃) δ ppm: 8.75 (s, 1H), 8.00 (s, 1H), 7.81 (s, 1H), 7.55 (s, 1H), 6.39 (s, 1H), 5.40-5.33 (m, 1H), 4.16-4.10 (m, 1H), 3.96 (s, 3H), 3.28 (s, 3H), 2.52-2.48 (m, 2H), 2.46-2.43 (m, 2H), 2.20 (s, 3H), 2.06 (t, J = 18.8 Hz, 3H). |

### Example 20

### N-(4-((2-(1,1-difluoroethyl)-6-ethylpyrimidin-4-yl)amino)-5-(2-methoxyethoxy)pyridin-2-yl)acetamide

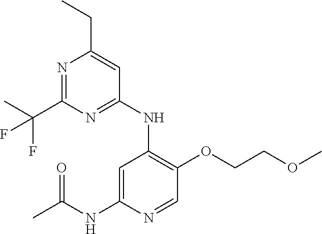 [SMILES: CCc1cc(Nc2cc(NC(C)=O)ncc2OCCOC)nc(C(C)(F)F)n1]

To a solution of N-(4-amino-5-(2-methoxyethoxy)pyridin-2-yl)acetamide hydrochloride (Preparation 63, 70.0 mg, 0.311 mmol) in dioxane (5.0 mL) was added 4-chloro-2-(1,1-difluoroethyl)-6-ethylpyrimidine (Preparation 94, 64.2 mg, 0.311 mmol), Cs₂CO₃ (303.8 mg, 0.932 mmol) and BrettPhos Pd G₃ (28.2 mg, 0.031 mmol). The mixture was stirred at 100° C. for 1 h under N₂. The mixture was concentrated and purified by prep-HPLC (Method B, Gradient 25 to 55%)

to give N-(4-((2-(1,1-difluoroethyl)-6-ethylpyrimidin-4-yl)amino)-5-(2-methoxyethoxy)pyridin-2-yl)acetamide (54.3 mg, 43.9% yield) as a white solid. LCMS m/z=396.2 [M+H]⁺. ¹H NMR: (400 MHz, CDCl₃) δ ppm: 9.11 (s, 1H), 8.16 (s, 2H), 7.92 (s, 1H), 6.78 (s, 1H), 4.20-4.18 (m, 2H), 3.75-3.73 (m, 2H), 3.51 (s, 3H), 2.81 (q, J=7.6 Hz, 2H), 2.20-2.11 (m, 6H), 1.33 (t, J=7.6 Hz, 3H).

### Example 21

### N-(4-((2-(1,1-difluoroethyl)-6-methoxypyrimidin-4-yl)amino)-5-(2-methoxyethoxy)pyridin-2-yl)acetamide

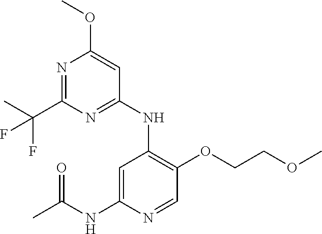 [SMILES: COCCOc1cnc(NC(C)=O)cc1Nc1cc(OC)nc(C(C)(F)F)n1]

N-(4-((2-(1,1-Difluoroethyl)-6-methoxypyrimidin-4-yl)amino)-5-(2-methoxyethoxy)pyridin-2-yl)acetamide was obtained, as a white solid, 7.3 mg, 18% yield, from 4-chloro-2-(1,1-difluoroethyl)-6-methoxypyrimidine (Preparation 97) and N-(4-amino-5-(2-methoxyethoxy)pyridin-2-yl)acetamide hydrochloride (Preparation 63), following the procedure described in Example 20. LCMS m/z=398.2 [M+H]⁺. ¹H NMR: (500 MHz, CDCl₃) δ ppm: 8.92 (s, 1H), 8.09-8.06 (m, 2H), 7.90 (s, 1H), 6.36 (s, 1H), 4.19-4.17 (m, 2H), 4.01 (s, 3H), 3.74-3.72 (m, 2H), 3.50 (s, 3H), 2.19 (s, 3H), 2.11 (t, J=18.5 Hz, 1H).

### Example 22

### N-(4-((2-(1,1-difluoroethyl)-6-methylpyrimidin-4-yl)amino)-5-(2-methoxyethoxy)pyridin-2-yl)acetamide

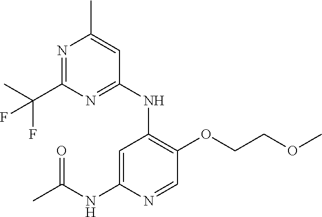 [SMILES: COCCOc1cnc(NC(C)=O)cc1Nc1cc(C)nc(C(C)(F)F)n1]

N-(4-((2-(1,1-difluoroethyl)-6-methylpyrimidin-4-yl)amino)-5-(2-methoxyethoxy)pyridin-2-yl)acetamide was obtained as a white solid, 42.2 mg, 41% yield, from 4-chloro-2-(1,1-difluoroethyl)-6-methylpyrimidine (Preparation 92) and N-(4-amino-5-(2-methoxyethoxy)pyridin-2-yl)acetamide hydrochloride (Preparation 63), following the procedure described in Example 20. LCMS m/z=382.2 [M+H]⁺ ¹H NMR: (500 MHz, CDCl₃) δ ppm: 9.12 (br s, 1H), 8.21-8.12 (m, 2H), 7.93 (s, 1H), 6.76 (s, 1H), 4.20-4.14 (m, 2H), 3.75-3.52 (m, 2H), 3.50 (s, 3H), 2.53 (s, 3H), 2.19-2.12 (m, 6H).

### Example 23 to 29

The compounds in the following table were prepared from 4-chloro-2-(1,1-difluoroethyl)-6-methylpyrimidine (Preparation 92) and the appropriate acetamide following a similar procedure to that described in Example 20

| Example | |
|---|---|
| No | Name, Structure, Acetamide, Data |
| 23 | N-(4-((2-(1,1-difluoroethyl)-6-methylpyrimidin-4-yl)amino)-5-(2,2,2- trifluoroethoxy)pyridin-2-yl)acetamide 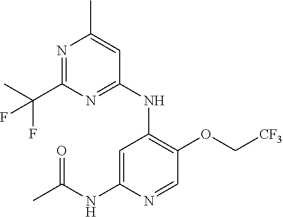 [SMILES: CC(=O)Nc1cc(Nc2cc(C)nc(C(C)(F)F)n2)c(OCC(F)(F)F)cn1] Acetamide: N-(4-amino-5-(2,2,2-trifluoroethoxy)pyridin-2-yl)acetamide hydrochloride (Preparation 69) Prep-HPLC (Method B, Gradient 20 to 50%) 35.3 mg, 24.8% yield as a white solid. LCMS m/z = 406.1 [M + H]⁺ ¹H NMR: (500 MHz, CDCl₃) δ ppm: 9.16 (s, 1H), 7.93 (s, 1H), 7.87 (s, 1H), 7.39 (s, 1H), 6.80 (s, 1H), 4.48 (q, J = 8.0 Hz, 2H), 2.56 (s, 3H), 2.21 (s, 3H), 2.15 (t, J = 19.0 Hz, 3H). |
| 24 | N-(5-(2,2-difluoroethoxy)-4-((2-(1,1-difluoroethyl)-6-methylpyrimidin-4- yl)amino)pyridin-2-yl)acetamide 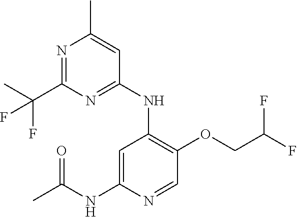 [SMILES: CC(=O)Nc1cc(Nc2cc(C)nc(C(C)(F)F)n2)c(OCC(F)F)cn1] Acetamide: N-(4-amino-5-(2,2-difluoroethoxy)pyridin-2-yl)acetamide hydrochloride (Preparation 70) Prep-HPLC (Method C, Gradient 36 to 66%) 40.0 mg, 19.9% yield as white solid. LCMS m/z = 388.1 [M + H]⁺ 1H NMR (400 MHZ; CDCl₃) δ ppm: 9.15 (s, 1H), 8.15 (br s, 1H), 7.84 (s, 1H), 7.47 (s, 1H), 6.85 (s, 1H), 6.30-6.01 (m, 1H), 4.37-4.29 (m, 2H), 2.56 (s, 3H), 2.22 (s, 3H), 2.15 (t, J = 18.8 Hz, 3H). |
| 25 | N-(4-((2-(1,1-difluoroethyl)-6-methylpyrimidin-4-yl)amino)-5-(2- (dimethylamino)ethoxy)pyridin-2-yl)acetamide 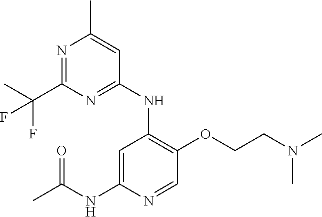 [SMILES: CC(=O)Nc1cc(Nc2cc(C)nc(C(C)(F)F)n2)c(OCCN(C)C)cn1] Acetamide: N-(4-amino-5-(2-(dimethylamino)ethoxy)pyridin-2-yl)acetamide hydrochloride (Preparation 71) Prep-HPLC (Method D, Gradient 5 to 25%). 42.78 mg, 34.5% yield) as a white solid. LCMS m/z = 395.3 [M + H]⁺. 1H NMR (400 MHz, CDCl₃) δ ppm: 9.76 (s, 1H), 9.21 (s, 1H), 8.48 (s, 1H), 8.27 (s, 1H), 7.83 (s, 1H), 6.90 (s, 1H), 4.19 (t, J = 4.0 Hz, 2H), 2.92 (s, 2H), 2.56 (s, 6H), 2.52 (s, 3H), 2.16 (t, J = 18.8 Hz, 6H). |
| 26 | N-(4-((2-(1,1-difluoroethyl)-6-methylpyrimidin-4-yl)amino)-5-ethoxypyridin- 2-yl)-2-methoxyacetamide 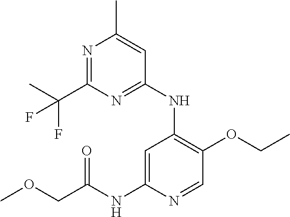 [SMILES: CCOc1cnc(NC(=O)COC)cc1Nc1cc(C)nc(C(C)(F)F)n1] Acetamide: N-(4-amino-5-ethoxypyridin-2-yl)-2-methoxyacetamide hydrochloride (Preparation 74) Prep-HPLC (Method C, Gradient 25 to 55%) 60 mg, 35.4% yield as a yellow solid. LCMS m/z = 382.1 [M + H]⁺ ¹H NMR: (400 MHz, CDCl₃) δ: ppm 9.07 (s, 1H), 8.74 (s, 1H), 7.85 (s, 1H), 7.52 (s, 1H), 6.86 (s, 1H), 4.20 (q, J = 6.8 Hz, 2H), 4.04 (s, 2H), 3.50 (s, 3H), 2.55 (s, 3H), 2.15 (t, J = 18.8 Hz, 3H), 1.51 (t, J = 6.8 Hz, 3H). |
| 27 | N-(4-((2-(1,1-difluoroethyl)-6-methylpyrimidin-4-yl)amino)-5-ethoxypyridin- 2-yl)-3-methoxypropanamide 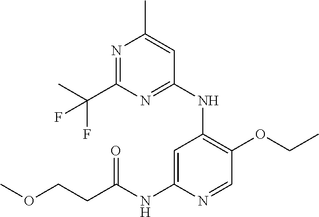 [SMILES: CCOc1cnc(NC(=O)CCOC)cc1Nc1cc(C)nc(C(C)(F)F)n1] Acetamide: N-(4-amino-5-ethoxypyridin-2-yl)-3-methoxypropanamide hydrochloride (Preparation 75) Prep-HPLC (Method B, Gradient 22 to 51%) 192.0 mg, 77.5% yield as yellow solid. LCMS m/z = 396.2 [M + H]⁺ ¹H NMR: (400 MHz, CDCl₃) δ ppm: 9.03 (s, 1H), 8.86 (br s, 1H), 7.81 (s, 1H), 7.55 (s, 1H), 6.90 (s, 1H), 4.19 (q, J = 6.8 Hz, 2H), 3.75 (t, J = 6.4 Hz, 2H), 3.45 (s, 3H), 2.67 (t, J = 6.4 Hz, 2H), 2.56 (s, 3H), 2.14 (t, J = 18.8 Hz, 3H), 1.50 (t, J = 6.8 Hz, 3H). |
| 28 | N-(4-((2-(1,1-difluoroethyl)-6-methylpyrimidin-4-yl)amino)-5-ethoxypyridin- 2-yl)cyclopropanecarboxamide 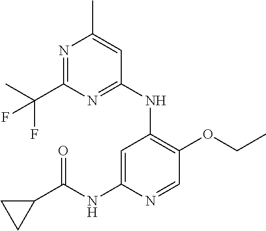 [SMILES: CCOc1cnc(NC(=O)C2CC2)cc1Nc1cc(C)nc(C(C)(F)F)n1] Acetamide: N-(4-amino-5-ethoxypyridin-2-yl)cyclopropanecarboxamide hydrochloride (Preparation 76) Prep-HPLC (Method C, 28 to 58%) 25.2 mg, 10.6% yield as a white solid. LCMS m/z = 378.1 [M + H]⁺ ¹H NMR: (400 MHz, CDCl₃) δ ppm: 8.94 (s, 1H), 8.19 (s, 1H), 7.80 (s, 1H), 7.51 (s, 1H), 6.89 (s, 1H), 4.17 (q, J = 7.2 Hz, 2H), 2.55 (s, 3H), 2.12 (t, J = 19.2 Hz, 3H), 1.55-1.52 (m, 4H), 1.10-1.07 (m, 2H), 0.92-0.85 (m, 2H). |
| 29 | N-(5-cyclopropyl-4-((2-(1,1-difluoroethyl)-6-methylpyrimidin-4- yl)amino)pyridin-2-yl)acetamide 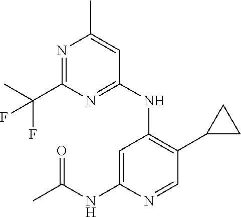 [SMILES: CC(=O)Nc1cc(Nc2cc(C)nc(C(C)(F)F)n2)c(C2CC2)cn1] Acetamide: N-(4-amino-5-cyclopropylpyridin-2-yl)acetamide hydrochloride (Preparation 81) Prep-HPLC (Method B, Gradient 22 to 52%) 12.0 mg, 15.7% yield as a white solid. LCMS m/z = 348.1 [M + H]⁺ 1H NMR: (400 MHz, CDCl₃) δ ppm: 8.67 (s, 1H), 8.02 (s, 2H), 7.47 (s, 1H), 7.00 (s, 1H), 2.56 (s, 3H), 2.21 (s, 3H), 2.15 (t, J = 18.8 Hz, 3H), 1.69-1.67 (m, 1H), 1.09-1.05 (m, 2H), 0.71-0.67 (m, 2H). |

### Example 30

### N-(5-(benzyloxy)-4-((2-(1,1-difluoroethyl)-6-methylpyrimidin-4-yl)amino)pyridin-2-yl)acetamide

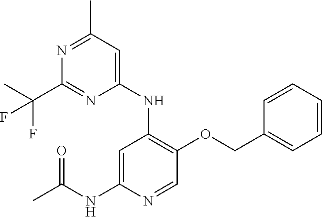 [SMILES: CC(=O)Nc1cc(Nc2cc(C)nc(C(C)(F)F)n2)c(OCc2ccccc2)cn1]

To a mixture of N-(4-amino-5-(benzyloxy)pyridin-2-yl)acetamide hydrochloride (Preparation 82, 250 mg, 0.851 mmol), Cs₂CO₃ (900 mg, 2.76 mmol) and dioxane (5 mL) which was degassed by bubbling N₂ for 15 mins at rt was added 4-chloro-2-(1,1-difluoroethyl)-6-methylpyrimidine (Preparation 92, 175 mg, 0.908 mmol) and BrettPhos Pd G3 (35 mg, 0.0386 mmol) and the reaction sealed and heated to 110° C. for 2 h. The cooled reaction mixture was diluted with water (10 mL) and extracted with EtOAc (3×10 mL). The combined organic layers were concentrated to dryness, then purified by silica gel chromatography (heptane to EtOAc) to give N-(5-(benzyloxy)-4-((2-(1,1-difluoroethyl)-6-methylpyrimidin-4-yl)amino)pyridin-2-yl)acetamide as a white solid, (53 mg, 15.1% yield). LCMS m/z=414.4 [M+H]⁺. 1H NMR (DMSO-d₆) δ: 10.22 (s, 1H), 9.28 (s, 1H), 8.96 (br s, 1H), 8.04 (s, 1H), 7.49 (br d, J=7.3 Hz, 2H), 7.38 (t, J=7.5 Hz, 2H), 7.33 (br d, J=7.6 Hz, 1H), 7.13 (s, 1H), 5.29 (s, 2H), 2.40 (s, 3H), 2.05 (s, 6H).

### Example 31

### N-(5-ethoxy-4-((2-(2-fluoropropan-2-yl)pyrimidin-4-yl)amino)pyridin-2-yl)acetamide

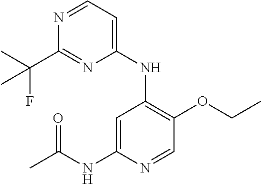 [SMILES: CCOc1cnc(NC(C)=O)cc1Nc1ccnc(C(C)(C)F)n1]

To a solution of N-(4-amino-5-ethoxypyridin-2-yl)acetamide hydrochloride (Preparation 62, 50.0 mg, 0.216 mmol) in DMF (2 mL) was added 4-chloro-2-(2-fluoropropan-2-yl)pyrimidine (37.7 mg, 0.216 mmol), Cs₂CO₃ (141 mg, 0.432 mmol), Pd₂(dba)₃ (19.8 mg, 0.0216 mmol) and Xantphos (12.5 mg, 0.216 mmol). The resulting mixture was stirred at 120° C. for 12 h under N₂. The mixture was concentrated and purified by prep-HPLC (Method C, Gradient 34% to 64%) to give N-(5-ethoxy-4-((2-(2-fluoropropan-2-yl)pyrimidin-4-yl)amino)pyridin-2-yl)acetamide (3.8 mg, 5.3% yield) as a white solid. LCMS m/z=334.2 [M+H]⁺. ¹H NMR: (400 MHz, CDCl₃) δ ppm: 9.04 (s, 1H), 8.52 (d, J=6.0 Hz, 1H), 7.81 (s, 1H), 7.75 (s, 1H), 7.52 (s, 1H), 6.86 (d, J=6.0 Hz, 1H), 4.20 (q, J=7.2 Hz, 2H), 2.20 (s, 3H), 1.89 (s, 3H), 1.84 (s, 3H), 1.51 (t, J=6.8 Hz, 3H).

### Example 32

### N-(4-((2-(1,1-difluoroethyl)-6-(2-methoxyethoxy)pyrimidin-4-yl)amino)-5-ethoxypyridin-2-yl)acetamide

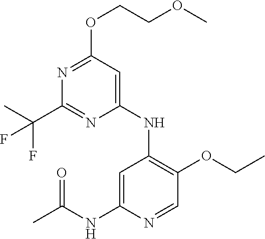 [SMILES: CCOc1cnc(NC(C)=O)cc1Nc1cc(OCCOC)nc(C(C)(F)F)n1]

N-(4-((2-(1,1-Difluoroethyl)-6-(2-methoxyethoxy)pyrimidin-4-yl)amino)-5-ethoxypyridin-2-yl)acetamide was obtained, 39 mg, 37% yield, as a white solid, using a similar method to that described in Example 31, from N-(4-amino-5-ethoxypyridin-2-yl)acetamide hydrochloride (Preparation 62) and 4-chloro-2-(1,1-difluoroethyl)-6-(2-methoxyethoxy)pyrimidine (Preparation 112). LCMS m/z=412.1 [M+H]⁺. ¹H NMR: (500 MHz, MeOH-d₄) δ ppm: 8.93 (s, 1H), 7.89 (s, 1H), 6.54 (s, 1H), 4.51-4.53 (m, 2H), 4.22 (q, J=7.0 Hz, 2H), 3.76-3.75 (m, 2H), 3.41 (s, 3H), 2.15 (s, 3H), 2.04 (t, J=18.5 Hz, 3H) 1.49 (t, J=7.0 Hz, 3H).

### Example 33

N-(4-((6-(1,1-difluoroethyl)pyrimidin-4-yl)amino)-5-ethoxypyridin-2-yl)acetamide

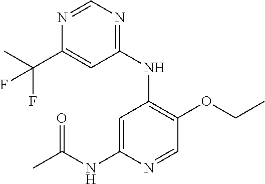 [SMILES: CCOc1cnc(NC(C)=O)cc1Nc1cc(C(C)(F)F)ncn1]

To a solution of N-(4-amino-5-ethoxypyridin-2-yl)acetamide hydrochloride (Preparation 62, 60.0 mg, 0.307 mmol) in dioxane (2 mL) was added 4-chloro-6-(1,1-difluoroethyl)pyrimidine (82.3 mg, 0.461 mmol), Xantphos (71.1 mg, 0.123 mmol), Pd₂(dba)₃ (56.3 mg, 0.0615 mmol) and Cs₂CO₃ (200.3 mg, 0.615 mmol). The resulting mixture was stirred at 100° C. for 1 h under N₂. The mixture was concentrated and purified by prep-HPLC (Method B, Gradient 31 to 61%) to give N-(4-((6-(1,1-difluoroethyl)pyrimidin-4-yl)amino)-5-ethoxypyridin-2-yl)acetamide (34.2 mg, 33.0% yield) as a yellow solid. LCMS m/z=338.0 [M+H]⁺. ¹H NMR: (400 MHz, CDCl₃) δ ppm: 9.26 (s, 1H), 8.96 (s, 1H), 7.83 (s, 1H), 7.80 (s, 1H), 7.59 (s, 1H), 7.16 (s, 1H), 4.20 (d, J=6.8 Hz, 2H), 2.21 (s, 3H), 2.00 (t, J=18.8 Hz, 3H), 1.51 (t, J=6.8 Hz, 3H).

### Examples 34 to 37

The compounds in the following table were prepared from N-(4-amino-5-ethoxypyridin-2-yl)acetamide hydrochloride (Preparation 62) or N-(4-amino-5-methoxypyridin-2-yl)acetamide hydrochloride (Preparation 61) and the appropriate substituted pyridine or pyrimidine (Het) following a similar procedure to that described in Example 33

| | |
|---|---|
| Example | |
| No | Name, Structure, Het, Data |

Acetamide: N-(4-amino-5-ethoxypyridin-2-yl)acetamide hydrochloride (Preparation 62)

| | |
|---|---|
| 34 | N-(4-((2-(1,1-difluoroethyl)-5-fluoropyrimidin-4-yl)amino)-5-ethoxypyridin-2- yl)acetamide 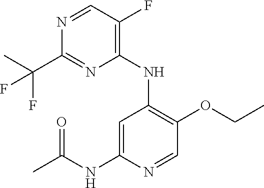 [SMILES: CCOc1cnc(NC(C)=O)cc1Nc1nc(C(C)(F)F)ncc1F] Het: 4-chloro-2-(1,1-difluoroethyl)-5-fluoropyrimidine (Preparation 109) Prep-HPLC (Method B, Gradient: 25 to 65%) 8.8 mg, 6.0% yield as a white solid. LCMS m/z = 356.1 [M + H]⁺. ¹H NMR: (500 MHz, CDCl₃) δ ppm: 9.45 (s, 1H), 8.40 (d, J = 2.5 Hz, 1H), 7.96 (s, 1H), 7.83-7.86 (m, 2H), 4.23 (q, J = 7.0 Hz, 2H), 2.26-2.19 (m, 6H), 1.52 (t, J = 7.0 Hz, 3H). |
| 35 | N-(4-((2-(1,1-difluoroethyl)-5-fluoro-6-methylpyrimidin-4-yl)amino)-5- ethoxypyridin-2-yl)acetamide 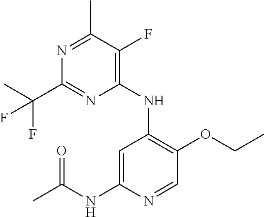 [SMILES: CCOc1cnc(NC(C)=O)cc1Nc1nc(C(C)(F)F)nc(C)c1F] Het: 4-chloro-2-(1,1-difluoroethyl)-5-fluoro-6-methylpyrimidine (Preparation 110). Prep-HPLC Method I (Gradient: 30% to 50%) followed by Method B (Gradient 33% to 63%). 16.8 mg, 13.2% yield as a white solid. LCMS m/z = 370.1 [M + H]⁺. ¹H NMR: (500 MHz, CDCl₃) δ ppm: 9.52 (s, 1H), 8.54 (br s, 1H), 7.97 (s, 1H), 7.77 (s, 1H), 4.22 (q, J = 7.0 Hz, 2H), 2.56 (d, J = 3.0 Hz, 3H), 2.17-2.25 (m, 6H), 1.53 (t, J = 7.0 Hz, 3H). |
| | Acetamide: N-(4-amino-5-methoxypyridin-2-yl)acetamide hydrochloride (Preparation 61) |
| 36 | N-(4-((2-(1,1-difluoroethyl)-6-ethylpyrimidin-4-yl)amino)-5-methoxypyridin- 2-yl)acetamide 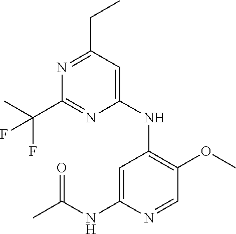 [SMILES: CCc1cc(Nc2cc(NC(C)=O)ncc2OC)nc(C(C)(F)F)n1] Het: 4-chloro-2-(1,1-difluoroethyl)-6-ethylpyrimidine (Preparation 94) Prep-HPLC (Method B, Gradient: 23 to 53%)40 mg, 29.4% yield as a white solid. LCMS m/z = 352.1 [M + H]⁺. ¹H NMR: (500 MHz, CDCl₃) δ ppm: 8.99 (s, 1H), 7.85 (s, 1H), 7.82 (s, 1H), 7.55 (s, 1H), 6.88 (s, 1H), 3.97 (s, 3H), 2.82 (q, J = 7.5 Hz, 2H), 2.20 (s, 3H), 2.14 (t, J = 18.5 Hz, 3H), 1.34 (t, J = 7.5 Hz, 3H) |
| 37 | N-(4-((2-(1,1-difluoroethyl)-6-(2-methoxyethoxy)pyrimidin-4-yl)amino)-5- methoxypyridin-2-yl)acetamide 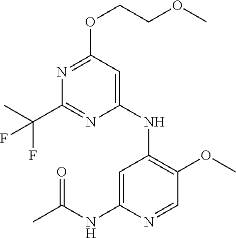 [SMILES: COCCOc1cc(Nc2cc(NC(C)=O)ncc2OC)nc(C(C)(F)F)n1] Het: 4-chloro-2-(1,1-difluoroethyl)-6-(2-methoxyethoxy)pyrimidine (Preparation 112) Prep-HPLC (Method B, Gradient: 26 to 56%) 35 mg, 11.1% yield as a white solid. LCMS m/z = 398.1 [M + H]⁺. ¹H NMR: (500 MHz, CDCl₃) δ ppm: 8.83 (s, 1H), 7.82 ( s, 1H), 7.80 (s, 1H), 7.48 (s, 1H), 6.42 (s, 1H), 4.56 (t, J = 5.0 Hz, 2H), 3.96 (s, 3H), 3.74 (t, J = 5.0 Hz, 2H), 3.43 (s, 3H), 2.19 (s, 3H), 2.09 (t, J = 18.5 Hz, 3H) |

### Example 38

### N-(5-cyclopropoxy-4-((2-(1,1-difluoroethyl)pyrimidin-4-yl)amino)pyridin-2-yl)acetamide

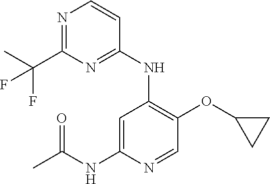 [SMILES: CC(=O)Nc1cc(Nc2ccnc(C(C)(F)F)n2)c(OC2CC2)cn1]

To a solution of N-(4-amino-5-cyclopropoxypyridin-2-yl)acetamide hydrochloride (Preparation 67, 70 mg, 0.34 mmol) in dioxane (2 mL) was added 4-chloro-2-(1,1-difluoroethyl)pyrimidine (66.35 mg, 0.37 mmol), Cs₂CO₃ (330.18 mg, 1.01 mmol), Pd₂(dba)₃ (61.86 mg, 0.07 mmol) and Xantphos (39.09 mg, 0.07 mmol) and the mixture was stirred at 120° C. for 16 h under N₂. The cooled mixture was concentrated in vacuo and the residue purified by Prep-HPLC (Method B, Gradient: 20 to 55%) to give N-(5-cyclopropoxy-4-((2-(1,1-difluoroethyl)pyrimidin-4-yl)amino)pyridin-2-yl)acetamide (10.2 mg, 8.6% yield) as a white solid. LCMS m/z=350.1 [M+H]⁺. ¹H NMR: (500 MHz, CDCl₃) δ: ppm 9.02 (s, 1H), 8.58 (d, J=5.5 Hz, 1H), 8.14 (s, 1H), 7.49 (s, 1H), 7.02 (d, J=6.0 Hz, 1H), 3.90-3.85 (m, 1H), 2.22 (s, 3H), 2.14 (t, J=18.5 Hz, 3H), 0.91-0.88 (m, 4H).

### Examples 39 to 44

The compounds in the following table were prepared from 4-chloro-2-(1,1-difluoroethyl)-6-methylpyrimidine (Preparation 92) and the appropriate substituted acetamide following a similar procedure to that described in Example 38.

| Example | |
|---|---|
| No | Name, Structure, Acetamide, Data |
| 39 | N-(4-((2-(1,1-difluoroethyl)-6-methylpyrimidin-4-yl)amino)-5- isopropoxypyridin-2-yl)acetamide 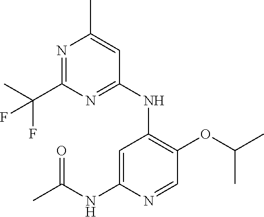 [SMILES: CC(=O)Nc1cc(Nc2cc(C)nc(C(C)(F)F)n2)c(OC(C)C)cn1] Acetamide: N-(4-amino-5-isopropoxypyridin-2-yl)acetamide hydrochloride (Preparation 66) Prep-HPLC (Method C, Gradient: 33 to 63%) 25 mg, 26.4% yield as a yellow solid. LCMS m/z = 366.1 [M + H]⁺ 1H NMR: (500 MHz, CDCl₃) δ ppm: 8.98 (s, 1H), 7.96 (s, 1H), 7.83 (s, 1H), 7.52 (s, 1H), 6.86 (s, 1H), 4.66-4.59 (m, 1H), 2.54 (s, 3H), 2.23-2.09 (m, 6H), 1.41 (d, J = 6.0 Hz, 6H) |
| 40 | N-(4-((2-(1,1-difluoroethyl)-6-methylpyrimidin-4-yl)amino)-5-((3- methoxycyclobutyl)methoxy)pyridin-2-yl)acetamide 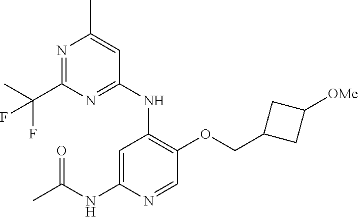 [SMILES: COC1CC(COc2cnc(NC(C)=O)cc2Nc2cc(C)nc(C(C)(F)F)n2)C1] Acetamide: N-(4-amino-5-((3-methoxycyclobutyl)methoxy)pyridin-2- yl)acetamide hydrochloride (Preparation 72) Prep-HPLC (Method B, Gradient: 25 to 55%) 15 mg, 12.6% yield as a yellow solid. LCMS m/z = 422.3 [M + H]⁺ ¹H NMR (400 MHz, CDCl₃) δ ppm 9.11 (s, 1H), 8.03 (s, 1H), 7.84 (s, 1H), 7.78 (s, 1H), 6.83 (s, 1H), 4.07 (d, J = 4.8 Hz, 2H), 3.94-3.86 (m, 1H), 3.27 (s, 3H), 2.58-2.44 (m, 6H), 2.22-2.11 (m, 6H), 1.98-1.90 (m, 2H). |
| 41 | N-(4-((2-(1,1-difluoroethyl)-6-methylpyrimidin-4-yl)amino)-5-methylpyridin- 2-yl)acetamide 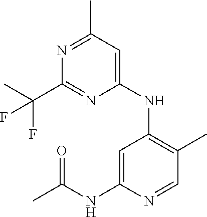 [SMILES: CC(=O)Nc1cc(Nc2cc(C)nc(C(C)(F)F)n2)c(C)cn1] Acetamide: N-(4-amino-5-methylpyridin-2-yl)acetamide hydrochloride (Preparation 77) Prep-HPLC (Method B, Gradient: 20 to 50%) 20 mg, 22.8% yield as a white solid. LCMS m/z = 322.1 [M + H]⁺. 1H NMR: (400 MHz, CDCl₃) δ ppm: 8.47 (s, 1H), 8.06 (s, 1H), 7.96 (s, 1H), 7.00 (s, 1H), 6.83 (s, 1H), 2.54 (s, 3H), 2.24 (s, 3H), 2.21 (s, 3H), 2.06 (t, J = 19.2 Hz, 3H). |
| 42 | N-(4-((2-(1,1-difluoroethyl)-6-methylpyrimidin-4-yl)amino)-5-ethylpyridin-2- yl)acetamide 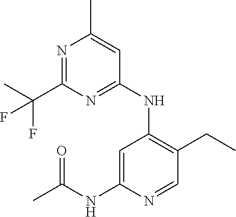 [SMILES: CCc1cnc(NC(C)=O)cc1Nc1cc(C)nc(C(C)(F)F)n1] Acetamide: N-(4-amino-5-ethylpyridin-2-yl)acetamide hydrochloride (Preparation 78) Prep-HPLC (Method B, Gradient: 20 to 50%) 75.1 mg, 48.3% yield as a white solid. LCMS m/z = 336.1 [M + H]⁺ ¹H NMR (500 MHz, CDCl₃) δ ppm: 8.43 (s, 1H), 8.35 (s, 1H), 8.08 (s, 1H), 6.99 (s, 1H), 6.90 (s, 1H), 2.62 (q, J = 7.5 Hz, 2H), 2.53 (s, 3H), 2.21 (s, 3H), 2.05 (t, J = 18.5 Hz, 3H), 1.26 (t, J = 7.5 Hz, 3H). |
| 43 | N-(4-((2-(1,1-difluoroethyl)-6-methylpyrimidin-4-yl)amino)-5-propylpyridin- 2-yl)acetamide 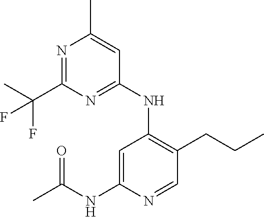 [SMILES: CCCc1cnc(NC(C)=O)cc1Nc1cc(C)nc(C(C)(F)F)n1] Acetamide: N-(4-amino-5-propylpyridin-2-yl)acetamide hydrochloride (Preparation 79) Prep-HPLC (Method B, Gradient: 24 to 53%) 34.8 mg, 28.6% yield as a white solid. LCMS m/z = 350.1 [M + H]⁺ ¹H NMR: (500 MHz, CDCl₃) δ ppm: 8.43 (s, 1H), 8.20 (s, 1H), 8.06 (s, 1H), 6.98 (s, 1H), 6.86 (s, 1H), 2.58-2.55 (m, 2H), 2.53 (s, 3H), 2.20 (s, 3H), 2.06 (t, J = 19.0 Hz, 3H), 1.66-1.60 (m, 2H), 0.99 (t, J = 7.5 Hz, 3H). |
| 44 | N-(4-((2-(1,1-difluoroethyl)-6-methylpyrimidin-4-yl)amino)-5- isopropylpyridin-2-yl)acetamide 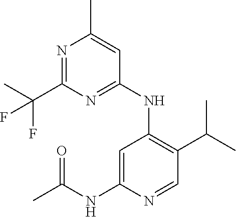 [SMILES: CC(=O)Nc1cc(Nc2cc(C)nc(C(C)(F)F)n2)c(C(C)C)cn1] Acetamide: N-(4-amino-5-isopropylpyridin-2-yl)acetamide hydrochloride (Preparation 80) Prep-HPLC (Method B, Gradient: 22 to 51%) 40 mg, 27.7% yield as a white solid. LCMS m/z = 350.1 [M + H]⁺ ¹H NMR: (400 MHz, CDCl₃) δ ppm: 8.36 (s, 1H), 8.16 (s, 1H), 8.07 (br s, 1H), 6.96 (s, 1H), 6.90 (s, 1H), 2.98-3.13 (m, 1H), 2.52 (s, 3H), 2.21 (s, 3H), 2.05 (t, J = 18.8 Hz, 3H), 1.31 (d, J = 7.2 Hz, 6H). |

### Example 45

### N-(5-cyclopropoxy-4-((2-(1,1-difluoroethyl)-6-methylpyrimidin-4-yl)amino)pyridin-2-yl)acetamide

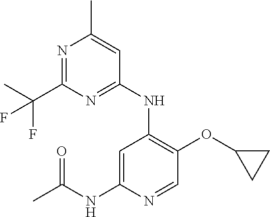 [SMILES: CC(=O)Nc1cc(Nc2cc(C)nc(C(C)(F)F)n2)c(OC2CC2)cn1]

To a solution of 4-chloro-2-(1,1-difluoroethyl)-6-methylpyrimidine (Preparation 92, 50.0 mg, 0.241 mmol), N-(4-amino-5-cyclopropoxypyridin-2-yl)acetamide hydrochloride (Preparation 67, 51.2 mg, 0.265 mmol) and K₃PO₄ (71.7 mg, 0.338 mmol) was added Pd₂(dba)₃ (22.1 mg, 0.024 mmol) and Xantphos (14.0 mg, 0.024 mmol) and the reaction was stirred at 80° C. for 16 h. The mixture was quenched with H₂O (30.0 mL) and extracted with EtOAc (20.0 mL×3). The combined organic layers were washed with brine (20 mL), dried over Na₂SO₄, filtered and concentrated under vacuum. The crude was purified by Prep-HPLC (Method B, Gradient: 25 to 55%) to give N-(5-cyclopropoxy-4-((2-(1,1-difluoroethyl)-6-methylpyrimidin-4-yl)amino)pyridin-2-yl)acetamide (20.0 mg, 22.8% yield) as a white solid. LCMS m/z=364.1 [M+H]⁺. 1H NMR (500 MHz, CDCl₃) δ ppm: 8.96 (s, 1H), 8.14 (s, 1H), 7.99 (s, 1H), 7.38 (s, 1H), 6.86 (s, 1H), 3.86 (s, 1H), 2.54 (s, 3H), 2.20 (s, 3H), 2.14 (t, J=18.5 Hz, 3H), 0.87 (s, 4H).

### Example 46

### N-(4-((2-(1,1-difluoroethyl)-6-methylpyrimidin-4-yl)amino)-5-methoxypyridin-2-yl)acetamide

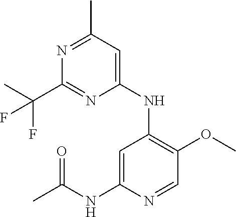 [SMILES: COc1cnc(NC(C)=O)cc1Nc1cc(C)nc(C(C)(F)F)n1]

To a solution of N-(4-amino-5-cyclopropoxypyridin-2-yl)acetamide hydrochloride (Preparation 67, 100.0 mg, 0.552 mmol), Pd₂(dba)₃ (50.6 mg, 0.0552 mmol), Xantphos (31.9 mg, 0.0552 mmol) and K₃PO₄ (234.3 mg, 1.10 mmol) in dioxane (2.0 mL) was added 4-chloro-2-(1,1-difluoroethyl)-6-methylpyrimidine (Preparation 92, 127.6 mg, 0.662 mmol) and the reaction was stirred at 80° C. for 5 h. The mixture was filtered and concentrated under vacuum to give the crude, which was purified by prep-HPLC (Method C, Gradient: 20 to 50%) to give
N-(4-((2-(1,1-difluoroethyl)-6-methylpyrimidin-4-yl)amino)-5-methoxypyridin-2-yl)acetamide (29.5 mg, 15.9% yield) as a white solid. LCMS m/z=338.1 [M+H]⁺ ¹H NMR: (400 MHz, CDCl₃) δ ppm: 9.02 (s, 1H), 8.11 (s, 1H), 7.81 (s, 1H), 7.55 (s, 1H), 6.85 (s, 1H), 3.96 (s, 3H), 2.54 (s, 3H), 2.20-2.10 (m, 6H).

### Example 47

### N-(4-((2-(1,1-difluoroethyl)-6-methylpyrimidin-4-yl)amino)-5-(methoxymethyl)pyridin-2-yl)acetamide

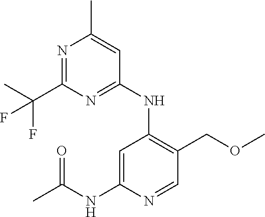 [SMILES: COCc1cnc(NC(C)=O)cc1Nc1cc(C)nc(C(C)(F)F)n1]

To a stirring solution of N-(4-chloro-5-(methoxymethyl)pyridin-2-yl)acetamide (Preparation 50, 16 mg, 0.0745 mmol) and 2-(1,1-difluoroethyl)-6-methylpyrimidin-4-amine (Preparation 93, 12.91 mg, 0.0745 mmol) in dioxane (1 mL) was added Cs₂CO₃ (48.57 mg, 0.149 mmol). The resulting mixture was degassed with N₂, BrettPhos Pd G3 (13.51 mg, 0.0149 mmol) was added and the reaction mixture was stirred at reflux at 100° C. for 48 h. The cooled mixture was filtered through Celite® and purified using Combiflash ((0-100% heptane/EtOAc: EtOH (3:1) with 2% NH₄OH)) to give N-(4-((2-(1,1-difluoroethyl)-6-methylpyrimidin-4-yl)amino)-5-(methoxymethyl)pyridin-2-yl)acetamide (2.9 mg, 11.1% yield). LCMS m/z=351.0 [M+H]⁺. ¹H NMR (500 MHz, CDCl₃) δ 8.91 (s, 1H), 8.35 (s, 1H), 8.03 (s, 1H), 8.01 (s, 1H), 6.81 (s, 1H), 4.53 (s, 2H), 3.42 (s, 3H), 2.54 (s, 3H), 2.21 (s, 3H), 2.12 (t, J=18.8 Hz, 3H)

### Example 48

### N-(4-((6-methoxy-5-(1-methyl-1H-pyrazol-3-yl)pyridin-2-yl)amino)-5-(methoxymethyl)pyridin-2-yl)acetamide

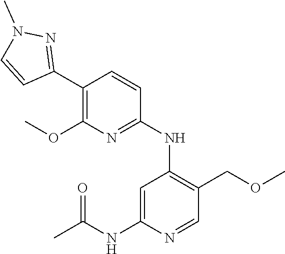 [SMILES: COCc1cnc(NC(C)=O)cc1Nc1ccc(-c2ccn(C)n2)c(OC)n1]

N-(4-((6-Methoxy-5-(1-methyl-1H-pyrazol-3-yl)pyridin-2-yl)amino)-5-(methoxymethyl)pyridin-2-yl)acetamide was obtained, 15.7 mg, 18.5% yield, from N-(4-chloro-5-(methoxymethyl)pyridin-2-yl)acetamide (Preparation 50) and 6-methoxy-5-(1-methyl-1H-pyrazol-3-yl)pyridin-2-amine (Preparation 123) following the procedure described in Example 47. LCMS m/z=382.0 [M+H]⁺. 1H NMR (500 MHz, CDCl₃) δ 9.07 (s, 1H), 8.58 (s, 1H), 8.17 (d, J=8.0 Hz, 1H), 8.08 (s, 1H), 7.90 (s, 1H), 7.37 (d, J=2.2 Hz, 1H), 6.77 (d, J=2.2 Hz, 1H), 6.51 (d, J=8.1 Hz, 1H), 4.54 (s, 2H), 4.15 (s, 3H), 3.94 (s, 3H), 3.40 (s, 3H), 2.17 (s, 3H).

### Example 49

### N-(4-((2-(1,1-difluoroethyl)-6-methylpyrimidin-4-yl)amino)-5-(ethoxy-d₅)pyridin-2-yl)acetamide

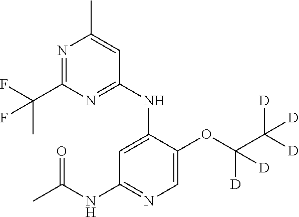 [SMILES: [2H]C([2H])([2H])C([2H])([2H])Oc1cnc(NC(C)=O)cc1Nc1cc(C)nc(C(C)(F)F)n1]

A mixture of N-(4-amino-5-(ethoxy-d₅)pyridin-2-yl)acetamide hydrochloride (Preparation 83, 1 g, 4.22 mmol), Cs₂CO₃ (3 g, 9.21 mmol), DMA (10 mL), DIPEA (742.0 mg, 5.74 mmol) and 4-chloro-2-(1,1-difluoroethyl)-6-methylpyrimidine (Preparation 92, 1.22 g, 6.34 mmol) was degassed while stirring for 30 mins at rt and bubbling N₂. MolDalPhos Pd G3 (350 mg, 420 μmol) was added, the vial sealed and heated to 100° C. The reaction mixture was diluted with EtOAc (20 mL), filtered through Celite®, rinsing with EtOAc (20 mL) and the filtrate concentrated to dryness. The crude material was purified by silica gel chromatography (80 g, heptane to EtOAc) to give an off-white solid. The obtained material was treated with EtOAc (25 mL) and diluted with heptane (200 mL). The mixture was heated to reflux, then stirred while cooling to rt over 2 h. The resulting white crystalline solids were collected via filtration to give N-(4-((2-(1,1-difluoroethyl)-6-methylpyrimidin-4-yl)amino)-5-(ethoxy-d5)pyridin-2-yl)acetamide (985 mg, 65.4% yield). LCMS m/z=357.1 [M+H]⁺. ¹H NMR (DMSO-d₆) δ: 10.22 (br s, 1H), 9.11 (s, 1H), 9.00 (br s, 1H), 8.01 (s, 1H), 7.15 (s, 1H), 2.40 (s, 3H), 2.05 (m, 6H).

### Example 50

### N-(4-((2-(1,1-difluoroethyl)-6-methoxypyrimidin-4-yl)amino)-5-(1-methyl-1H-pyrazol-3-yl)pyridin-2-yl)acetamide

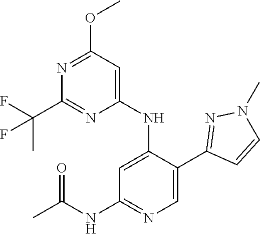 [SMILES: COc1cc(Nc2cc(NC(C)=O)ncc2-c2ccn(C)n2)nc(C(C)(F)F)n1]

To a solution of N-(2-chloro-5-(1-methyl-1H-pyrazol-3-yl)pyridin-4-yl)-2-(1,1-difluoroethyl)-6-methoxypyrimidin-4-amine (Preparation 119, 30 mg, 0.0789 mmol) in dioxane (3 mL) was added Cs₂CO₃ (30 mg, 0.092 mmol), acetamide (30 mg, 0.508 mmol) and BrettPhos Pd G3 (30 mg, 0.033 mmol), the vial sealed and heated at 90° C. for 20 h. The cooled reaction was diluted with water (10 mL), extracted with EtOAc (3×10 mL) and the combined organic layer was concentrated to dryness. The residue was purified by reverse phase prep-HPLC (Method J, Gradient: 5% to 60%), to give N-(4-((2-(1,1-difluoroethyl)-6-methoxypyrimidin-4-yl)amino)-5-(1-methyl-1H-pyrazol-3-yl)pyridin-2-yl)acetamide (3.40 mg, 10.7% yield) as a white solid. LCMS m/z=404.3 [M+H]⁺.

### Example 51

### N-(4-((2-(1,1-difluoroethyl)-6-isopropylpyrimidin-4-yl)amino)-5-ethoxypyridin-2-yl)acetamide

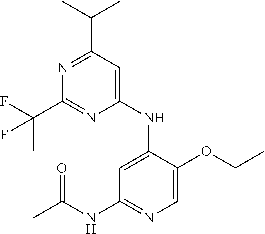 [SMILES: CCOc1cnc(NC(C)=O)cc1Nc1cc(C(C)C)nc(C(C)(F)F)n1]

To a mixture of N-(4-((2-(1,1-difluoroethyl)-6-(prop-1-en-2-yl)pyrimidin-4-yl)amino)-5-ethoxypyridin-2-yl)acetamide (Preparation 122, 50 mg, 0.132 mmol) in MeOH (50 mL) was added Pd/C (42.30 mg, 39.75 mol, 10% purity) and the reaction stirred at 20° C. under H₂ (15 psi) for 1 h. The reaction mixture was filtered and concentrated in vacuo. The crude was purified by prep-HPLC (Method B, Gradient: 30 to 60%) to give N-(4-((2-(1,1-difluoroethyl)-6-isopropylpyrimidin-4-yl)amino)-5-ethoxypyridin-2-yl)acetamide (21.7 mg, 43.2% yield) as a white solid. LCMS m/z=380.2 [M+H]⁺ 1H NMR: (500 MHz, DMSO-d₆) δ ppm: 10.20 (s, 1H), 9.10 (s, 1H), 9.03 (s, 1H), 8.00 (s, 1H), 7.22 (s, 1H), 4.19 (q, J=7.0 Hz, 2H), 2.95-2.86 (m, 1H), 2.12-2.04 (m, 6H), 1.40 (t, J=7.0 Hz, 3H), 1.24 (d, J=6.5 Hz, 6H).

### Example 52

### N-(4-((2-(1,1-difluoroethyl)-6-isopropylpyrimidin-4-yl)amino)-5-methoxypyridin-2-yl)acetamide

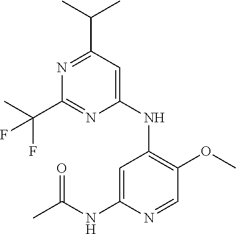 [SMILES: COc1cnc(NC(C)=O)cc1Nc1cc(C(C)C)nc(C(C)(F)F)n1]

To a mixture of N-(4-((2-(1,1-difluoroethyl)-6-(prop-1-en-2-yl)pyrimidin-4-yl)amino)-5-methoxypyridin-2-yl)acetamide (Preparation 121, 50 mg, 0.138 mmol) in MeOH (50 mL) was added Pd/C (43.9 mg, 0.0413 mmol, 10% purity) at 20° C. The reaction mixture was stirred under H₂ (15 psi) at 20° C. for 1 h. The mixture was filtered and concentrated to give a crude, which was purified by prep-HPLC (Method B, Gradient: 32 to 62%) to give N-(4-((2-(1,1-difluoroethyl)-6-isopropylpyrimidin-4-yl)amino)-5-methoxypyridin-2-yl)acetamide (25.9 mg, 51.5% yield) as a white solid. LCMS m/z=366.2 [M+H]⁺. ¹H NMR: (500 MHz, DMSO-d₆) δ ppm: 10.21 (s, 1H), 9.27 (s, 1H), 9.08 (s, 1H), 8.01 (s, 1H), 7.24 (s, 1H), 3.94 (s, 3H), 2.92-2.86 (m, 1H), 2.13-2.04 (m, 6H), 1.23 (d, J=6.5 Hz, 6H).

### Example 53

### ((2-(1,1-difluoroethyl)-6-(3-hydroxy-3-methylcyclobutoxy)pyrimidin-4-yl)amino)-5-ethoxypyridin-2-yl)acetamide

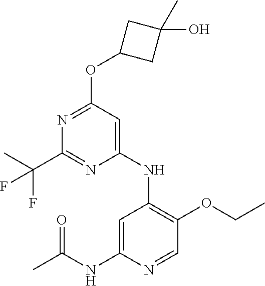 [SMILES: CCOc1cnc(NC(C)=O)cc1Nc1cc(OC2CC(C)(O)C2)nc(C(C)(F)F)n1]

To a solution of N-(4-((6-(3-((tert-butyldimethylsilyl)oxy)-3-methylcyclobutoxy)-2-(1,1-difluoroethyl)pyrimidin-4-yl)amino)-5-ethoxypyridin-2-yl)acetamide (Preparation 120, 80.0 mg, 0.145 mmol) in DCM (1 mL) was added HCl/dioxane (4 M, 2 mL). The mixture was stirred at 25° C. for 1 h. The mixture was concentrated in vacuo and the crude product purified by prep-HPLC (Method D, Gradient: 15 to 35%) to give N-(4-((2-(1,1-difluoroethyl)-6-(3-hydroxy-3-methylcyclobutoxy)pyrimidin-4-yl)amino)-5-ethoxypyridin-2-yl)acetamide (22.1 mg, 34.8% yield) as a white solid. LCMS m/z=438.1 [M+H]⁺. ¹H NMR: (400 MHz, CDCl₃) δ ppm: 9.06 (s, 1H), 7.65 (s, 2H), 6.43 (s, 1H), 4.99-4.91 (m, 1H), 4.18 (q, J=6.8 Hz, 2H), 2.74-2.68 (m, 2H), 2.33-2.25 (m, 2H), 2.25 (s, 3H), 2.08 (t, J=18.8 Hz, 3H), 1.52 (t, J=6.8 Hz, 3H), 1.44 (s, 3H).

### Example 54

### N-(4-((2-(1,1-difluoroethyl)-6-methylpyrimidin-4-yl)(methyl)amino)-5-ethoxypyridin-2-yl)acetamide

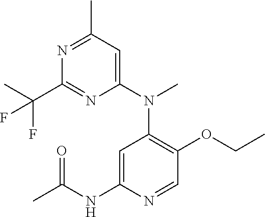 [SMILES: CCOc1cnc(NC(C)=O)cc1N(C)c1cc(C)nc(C(C)(F)F)n1]

To a solution of N-(4-((2-(1,1-difluoroethyl)-6-methylpyrimidin-4-yl)amino)-5-ethoxypyridin-2-yl)acetamide (Example 10, 60.0 mg, 0.171 mmol) in MeCN (3.0 mL) was added CH₃I (48.5 mg, 0.342 mmol) and K₂CO₃ (70.8 mg, 0.512 mmol) and the reaction was stirred at 70° C. for 4 h. The reaction mixture was concentrated in vacuo and the residue purified by prep-HPLC (Method D, Gradient: 30 to 60%) to give N-(4-((2-(1,1-difluoroethyl)-6-methylpyrimidin-4-yl)(methyl)amino)-5-ethoxypyridin-2-yl)acetamide (38.4 mg, 61.5% yield) as a white solid. LCMS m/z=366.1 [M+H]⁺. ¹H NMR: (400 MHz, CDCl₃) δ ppm: 8.14 (s, 1H), 8.02 (s, 1H), 7.85 (s, 1H), 6.13 (s, 1H), 4.07 (q, J=6.8 Hz, 2H), 3.45 (s, 3H), 2.36 (s, 3H), 2.20 (s, 3H), 2.00 (t, J=18.8 Hz, 3H), 1.27 (t, J=6.8 Hz, 3H).

### Example 55

### N-(4-((2-(1,1-difluoroethyl)-6-vinylpyrimidin-4-yl)amino)-5-(2-methoxyethoxy)pyridin-2-yl)acetamide

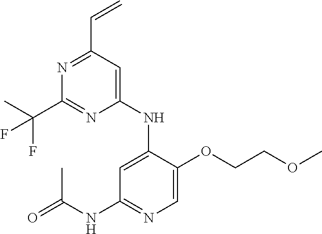 [SMILES: C=Cc1cc(Nc2cc(NC(C)=O)ncc2OCCOC)nc(C(C)(F)F)n1]

To a solution of N-(4-amino-5-(2-methoxyethoxy)pyridin-2-yl)acetamide hydrochloride (Preparation 63, 0.21 g, 0.80 mmol) and 4-chloro-2-(1,1-difluoroethyl)-6-vinylpyrimidine (Preparation 125, 191 mg, 0.93 mmol) in DMF (4 mL) was added BrettPhos Pd G3 (84 mg, 93 mol) and Cs₂CO₃ (609 mg, 1.87 mmol). The reaction mixture was sparged with N₂ for 5 mins then stirred at 70° C. for 2 h under N₂. The mixture was diluted with EtOAc, filtered and concentrated under reduced pressure. The residue was purified by silica gel chromatography (0-20% MeOH in DCM) and then repurified by silica gel chromatography (0-100% 3:1 EtOAc-EtOH+2% NH₄OH in heptanes) to give N-(4-((2-(1,1-difluoroethyl)-6-vinylpyrimidin-4-yl)amino)-5-(2-methoxyethoxy)pyridin-2-yl)acetamide (60 mg, 18% yield) as pale yellow solid. LCMS m/z=394.2 [M+H]⁺. ¹H NMR (400 MHz, MeOH-d₄) δ ppm 9.14 (s, 1H), 7.98 (s, 1H), 7.13 (s, 1H), 6.78 (dd, J=17.26, 10.76 Hz, 1H), 6.45-6.52 (m, 1H), 5.70 (dd, J=10.76, 1.25 Hz, 1H), 4.27-4.31 (m, 2H), 3.80-3.85 (m, 2H), 3.49 (s, 3H), 2.16 (s, 3H), 2.10 (t, J=18.76 Hz, 3H).

### Example 56

### N-(4-((6-(1,1-difluoroethyl)pyridin-2-yl)amino)-5-(2-methoxyethoxy)pyridin-2-yl)acetamide

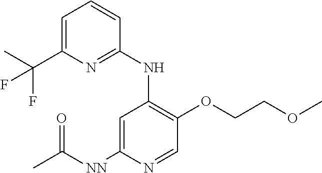

To a solution of N-(4-amino-5-(2-methoxyethoxy)pyridin-2-yl)acetamide (Preparation 63, 100 mg, 0.382 mmol, HCl salt) and 2-bromo-6-(1,1-difluoroethyl)pyridine (84.8 mg, 0.382 mmol) in dioxane (5.0 mL) were added Cs₂CO₃ (373 mg, 1.15 mmol) and BrettPhos Pd G₃ (34.6 mg, 38.2 umol) at 25° C. The reaction mixture was stirred at 100° C. for 1 h under N₂, then concentrated under reduced pressure. The residue was purified by prep-HPLC (Method B, Gradient 25-55%) to give N-(4-((6-(1,1-difluoroethyl)pyridin-2-yl)amino)-5-(2-methoxyethoxy)pyridin-2-yl)acetamide (31.0 mg, 22.1% yield) as a white solid. LCMS m/z=367.1 [M+H]⁺. ¹H NMR: (400 MHz, CDCl₃) δ ppm: 9.18 (br s, 1H), 8.35 (br s, 1H), 8.00 (s, 1H), 7.83 (s, 1H), 7.72 (t, J=8.0 Hz, 1H), 7.27 (d, J=7.6 Hz, 1H), 6.97 (d, J=8.4 Hz, 1H), 4.19-4.22 (m, 2H), 3.74-3.77 (m, 2H), 3.50 (s, 3H), 2.21 (s, 3H), 2.17 (t, J=19.2 Hz, 3H).

### Examples 57 to 62

The compounds in the following table were prepared from the corresponding ether substituted pyridines (SM) and 4-chloro-2-(1,1-difluoroethyl)-6-methylpyrimidine (Preparation 92) following a similar procedure to that described in Example 56

| Example | |
|---|---|
| No | Name, Structure, Starting Material (SM), Data |
| 57 | N-(4-((2-(1, 1-difluoroethyl)-6-methylpyrimidin-4-yl)amino)-5-((1- (trifluoromethyl)cyclopropyl)methoxy)pyridin-2-yl)acetamide 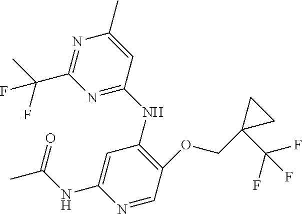 [SMILES: CC(=O)Nc1cc(Nc2cc(C)nc(C(C)(F)F)n2)c(OCC2(C(F)(F)F)CC2)cn1] SM: N-(4-amino-5-((1-(trifluoromethyl)cyclopropyl)methoxy)pyridin-2- yl)acetamide (Preparation 252) 20.7 mg, 11.6% yield as a white solid. Prep-HPLC (Method B, Gradient 33- 63%) LCMS m/z = 446.2 [M + H]⁺. ¹H NMR: (500 MHz, CDCl₃) CDCl₃ ppm: 9.28 (br s, 1H), 8.05 (br s, 1H), 7.74 (s, 1H), 7.62 (s, 1H), 6.67 (s, 1H), 4.16 (s, 2H), 2.56 (s, 3H), 2.14-2.22 (m, 6H), 1.25-1.28 (m, 2H), 0.94-0.96 (m, 2H). |
| 58 | N-(4-((2-(1,1-difluoroethyl)-6-methylpyrimidin-4-yl)amino)-5-((1- methylcyclopropyl)methoxy)pyridin-2-yl)acetamide 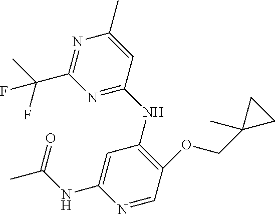 SM: N-(4-amino-5-((1-methylcyclopropyl)methoxy)pyridin-2-yl)acetamide (Preparation 254) 65.2 mg, 37.7% yield as a white solid. Prep-HPLC (Method B, Gradient 30- 60%) LCMS m/z = 367.1 [M + H]⁺. ¹H NMR: (400 MHz, CDCl₃) CDCl₃ ppm: 9.04 (br s, 1H), 7.89 (br s, 1H), 7.77 (s, 1H), 7.58 (s, 1H), 6.82 (s, 1H), 3.87 (s, 2H), 2.56 (s, 3H), 2.11-2.21 (m, 6H), 1.28 (s, 3H), 0.56-0.59 (m, 2H), 0.50-0.53 (m, 2H). |
| 59 | N-(4-((2-(1, 1-difluoroethyl)-6-methylpyrimidin-4-yl)amino)-5-((1- methoxypropan-2-yl)oxy)pyridin-2-yl)acetamide 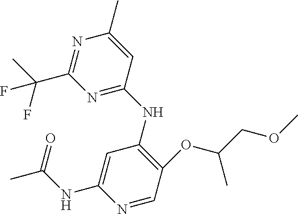 [SMILES: COCC(C)Oc1cnc(NC(C)=O)cc1Nc1cc(C)nc(C(C)(F)F)n1] SM: N-(4-amino-5-((1-methoxypropan-2-yl)oxy)pyridin-2-yl)acetamided (Preparation 255) 108 mg, 32.7% yield as a white solid. Prep-HPLC (Method B, Gradient 25- 55%) LCMS m/z = 396.2 [M + H]⁺. ¹H NMR: (500 MHz, CDCl₃) CDCl₃ ppm: 9.18 (s, 1H), 8.72 (s, 1H), 7.92-7.94 (m, 2H), 6.71 (s, 1H), 4.06-4.10 (m, 1H), 3.52-3.62 (m, 5H), 2.53 (s, 3H), 2.13-2.21 (m, 6H), 1.36 (d, J-6.5 Hz, 3H). |
| 60 | N-(4-((2-(1,1-difluoroethyl)-6-methylpyrimidin-4-yl)amino)-5-(2- methoxypropoxy)pyridin-2-yl)acetamide 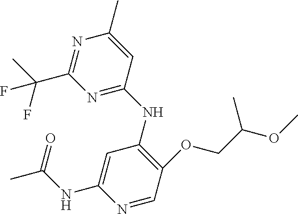 [SMILES: COC(C)COc1cnc(NC(C)=O)cc1Nc1cc(C)nc(C(C)(F)F)n1] SM: N-(4-amino-5-(2-methoxypropoxy)pyridin-2-yl)acetamide (Preparation 256) 70.2 mg, 34.3% yield as a white solid. Prep-HPLC (Method B, Gradient 35- 65%) LCMS m/z = 396.1 [M + H]⁺. ¹H NMR: (500 MHz, CDCl₃) CDCl₃ ppm: 9.08 (s, 1H), 8.20 (s, 1H), 7.88 (s, 1H), 7.77 (s, 1H), 6.73 (s, 1H), 4.05 (m, 1H), 3.90(m, 1H), 3.75 (m, 1H), 3.47 (s, 3H), 2.52 (s, 3H), 2.10-2.18 (m, 6H), 1.24 (d, J = 6.5 Hz, 3H). |
| 61 | N-(4-((2-(1, 1-difluoroethyl)-6-methylpyrimidin-4-yl)amino)-5-(oxetan-3- ylmethoxy)pyridin-2-yl)acetamide 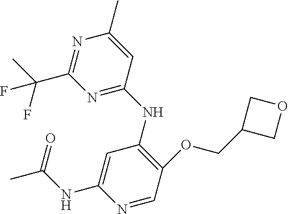 [SMILES: CC(=O)Nc1cc(Nc2cc(C)nc(C(C)(F)F)n2)c(OCC2COC2)cn1] SM: N-(4-amino-5-(oxetan-3-ylmethoxy)pyridin-2-yl)acetamide (Preparation 266) 45 mg, 8.73% yield as a white solid. Prep-HPLC (Method B, Gradient 29-59%) LCMS m/z = 394.1 [M + H]⁺. ¹H NMR: (500 MHz, DMSO-d₆) CDCl₃ ppm: 10.26 (s, 1H), 9.07 (s, 1H) 8.95 (s, 1H), 8.07 (s, 1H), 7.06 (s, 1H), 4.69-4.71 (m, 2H), 4.40 (t, J = 6.0 Hz, 2H), 4.36 (d, J = 7.0 Hz, 2H), 3.38-3.44 (m, 1H), 2.40 (s, 3H), 2.01-2.10 (m, 6H). |
| 62 | N-(5-(cyclobutylmethoxy)-4-((2-(1,1-difluoroethyl)-6-methylpyrimidin-4- yl)amino)pyridin-2-yl)acetamide 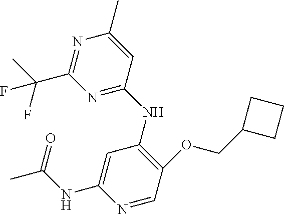 [SMILES: CC(=O)Nc1cc(Nc2cc(C)nc(C(C)(F)F)n2)c(OCC2CCC2)cn1] SM: N-(4-amino-5-(cyclobutylmethoxy)pyridin-2-yl)acetamide (Preparation 258) 55 mg, 38.2% yield as a white solid. Prep-HPLC (Method G, Gradient 37-57%) LCMS m/z = 392.1 [M + H]⁺. ¹H NMR: (400 MHz, CDCl₃) CDCl₃ ppm: 9.03 (s, 1H), 8.04 (br s, 1H), 7.80 (s, 1H), 7.50 (s, 1H), 6.82 (s, 1H), 4.08 (d, J = 7.2 Hz, 2H), 2.80-2.86 (m, 1H), 2.55 (s, 3H), 1.85-1.21 (m, 12H). |

### Examples 63 to 73

The compounds in the following table were prepared from the corresponding ether substituted pyridines (SM) and the appropriate pyridine or pyrimidine (Het) following a similar procedure to that described in Example 56.

| Example | |
|---|---|
| No | Name, Structure, starting material (SM), Het, Data |
| 63 | N-(4-((6-(1,1-difluoroethyl)-4-methylpyridin-2-yl)amino)-5-(2- |
| | methoxyethoxy)pyridin-2-y1)acetamide |
| | 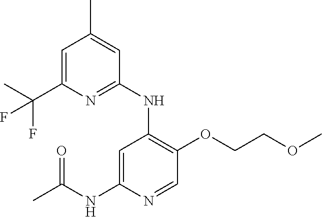 [SMILES: COCCOc1cnc(NC(C)=O)cc1Nc1cc(C)cc(C(C)(F)F)n1] |
| | SM: N-(4-amino-5-(2-methoxyethoxy)pyridin-2-yl)acetamide (Preparation 63) |
| | Het: 2-chloro-6-(1,1-difluoroethyl)-4-methylpyridine (Preparation 128) |
| | 52 mg, 35.8% yield as a white solid. Prep-HPLC (Method B, Gradient 33-60%) |
| | LCMS m/z = 381.1 [M + H]⁺. ¹H NMR: (500 MHz, CDCl₃) δ ppm: 9.14 (s, 1H), |
| | 8.11 (s, 1H), 7.84 (s, 1H), 7.09 (s, 1H), 6.77 (s, 1H), 4.17-4.20 (m, 2H), 3.73- |
| | 3.75 (m, 2H), 3.49 (s, 3H), 2.40 (s, 3H), 2.12-2.20 (m, 6H). |
| 64 | N-(4-((6-(1,1-difluoroethyl)pyridin-2-yl)amino)-5-(2-(pyrrolidin-1- |
| | yl)ethoxy)pyridin-2-yl)acetamide |
| | 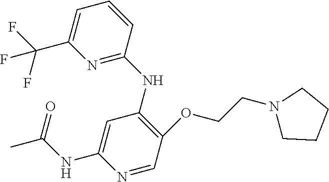 [SMILES: CC(=O)Nc1cc(Nc2cccc(C(F)(F)F)n2)c(OCCN2CCCC2)cn1] |
| | SM: N-(4-amino-5-(2-(pyrrolidin-1-yl)ethoxy)pyridin-2-yl)acetamide |
| | (Preparation 257) |
| | Het: 2-bromo-6-(1,1-difluoroethyl)pyridine |
| | 63.8 mg, 33.8% yield as a white solid. Prep-HPLC (Method K, Gradient 47- |
| | 78%) LCMS m/z = 406.4 [M + H]⁺. ¹H NMR: (500 MHz, CDCl₃) δ ppm: 9.15 (s, |
| | 1H), 8.82 (s, 1H), 8.15 (s, 1H), 7.77 (s, 1H), 7.64-7.68 (m, 1H), 7.19-7.24 (m, |
| | 2H), 4.21 (s, 2H), 3.02 (s, 2H), 2.85 (br s, 4H), 2.11-2.20 (m, 6H), 1.94 (br s, |
| | 4H). |
| 65 | N-(4-((6-(1,1-difluoroethyl)pyridin-2-y1)amino)-5-(2- |
| | (dimethylamino)ethoxy)pyridin-2-yl)acetamide |
| | 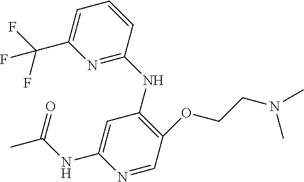 [SMILES: CC(=O)Nc1cc(Nc2cccc(C(F)(F)F)n2)c(OCCN(C)C)cn1] |
| | SM: N-(4-amino-5-(2-(dimethylamino)ethoxy)pyridin-2-yl)acetamide |
| | (Preparation 71) |
| | Het: 2-bromo-6-(1,1-difluoroethyl)pyridine |
| | 11.4 mg, 10.2% yield as a white solid. Prep-HPLC (Method K, Gradient 39- |
| | 69%) |
| | LCMS m/z = 380.1 [M + H]⁺. ¹H NMR: (400 MHz, CDCl₃) δ ppm: 9.23 (s, 1H), |
| | 9.14 (s, 1H), 7.97 (s, 1H), 7.87 (s, 1H), 7.67 (t, J = 8.4 Hz, 1H), 7.21 (d, J = 7.6 |
| | Hz, 1H), 6.97 (d, J = 8.4 Hz, 1H), 4.12 (t, J = 5.2 Hz, 2H), 2.69 (t, J = 5.2 Hz, 2H), |
| | 2.40 (s, 6H), 2.17 (m, 6H). |
| 66 | N-(4-((6-cyclopropyl-2-(1,1-difluoroethyl)pyrimidin-4-yl)amino)-5-(2- |
| | methoxyethoxy)pyridin-2-yl)acetamide |
| | 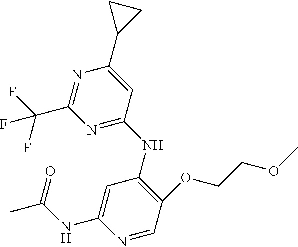 [SMILES: COCCOc1cnc(NC(C)=O)cc1Nc1cc(C2CC2)nc(C(F)(F)F)n1] |
| | SM: N-(4-amino-5-(2-methoxyethoxy)pyridin-2-yl)acetamide (Preparation 63) |
| | Het: 4-chloro-6-cyclopropyl-2-(1,1-difluoroethyl)pyrimidine (Preparation 155) |
| | 65 mg, 22.0% yield as a white solid. Prep-HPLC (Method B, Gradient 32-59%) |
| | LCMS m/z = 408.1 [M + H]⁺. ¹H NMR: (500 MHz, CDCl₃) δ ppm: 9.04 (s, 1H), |
| | 8.08 (s, 1H), 7.97 (s, 1H), 7.89 (s, 1H), 6.78 (s, 1H), 4.19-4.21 (m, 2H), 3.73- |
| | 3.76 (m, 2H), 3.51 (s, 3H), 2.21 (s, 3H), 2.09 (t, J = 18.5 Hz, 3H), 2.03-2.01 (m, |
| | 1H), 1.19-1.16 (m, 2H), 1.09-1.06 (m, 2H). |
| 67 | N-(4-((6-(1,1-difluoroethyl)pyrazin-2-yl)amino)-5-(2-methoxyethoxy)pyridin- |
| | 2-yl)acetamide |
| | 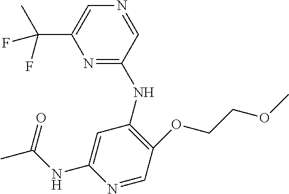 [SMILES: COCCOc1cnc(NC(C)=O)cc1Nc1cncc(C(C)(F)F)n1] |
| | SM: N-(4-amino-5-(2-methoxyethoxy)pyridin-2-yl)acetamide (Preparation 63) |
| | Het: 2-chloro-6-(1,1-difluoroethyl)pyrazine |
| | 11.5 mg, 8.81% yield as a white solid. Prep-HPLC (Method L, Gradient 20- |
| | 50%) |
| | LCMS m/z = 368.1 [M + H]⁺. ¹H NMR: (400 MHz, CDCl₃) δ ppm: 9.31 (s, 1H), |
| | 8.46 (s, 1H), 8.31 (s, 2H), 7.91 (s, 2H), 4.18-4.21 (m, 2H), 3.75-3.77 (m, 2H), |
| | 3.53 (s, 3H), 2.18-2.28 (m, 6H). |
| 68 | N-(4-((2-(1,1-difluoroethyl)-6-ethylpyrimidin-4-yl)amino)-5-(2- |
| | (difluoromethoxy)ethoxy)pyridin-2-yl)acetamide |
| | 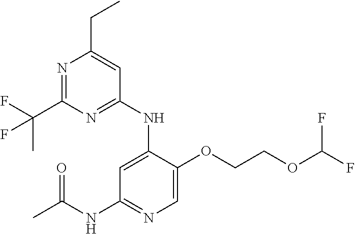 [SMILES: CCc1cc(Nc2cc(NC(C)=O)ncc2OCCOC(F)F)nc(C(C)(F)F)n1] |
| | SM: N-(4-amino-5-(2-(difluoromethoxy)ethoxy)pyridin-2-yl)acetamide |
| | (Preparation 261) |
| | Het: 4-chloro-2-(1,1-difluoroethyl)-6-ethylpyrimidine (Preparation 94) |
| | 39.0 mg, 26.9% yield as a white solid. Prep-HPLC (Method M, Gradient 23- |
| | 53%) |
| | LCMS m/z = 432.1 [M + H]⁺. ¹H NMR: (500 MHz, CDCl₃) δ ppm: 9.14 (s, 1H), |
| | 8.26 (s, 1H), 7.86 (s, 1H), 7.71 (s, 1H), 6.78 (s, 1H), 6.37 (t, J = 74.0 Hz, 1H), |
| | 4.27 (d, J = 3.5 Hz, 4H), 2.81 (q, J = 7.5 Hz, 2H), 2.11-2.20 (m, 6H), 1.33 (t, |
| | J = 7.5 Hz, 3H). |
| 69 | N-(4-((2-(1,1-difluoroethyl)-6-ethylpyrimidin-4-yl)amino)-5-(3- |
| | methoxypropoxy)pyridin-2-yl)acetamide |
| | 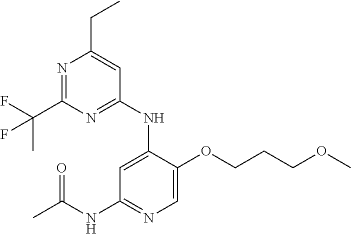 [SMILES: CCc1cc(Nc2cc(NC(C)=O)ncc2OCCCOC)nc(C(C)(F)F)n1] |
| | SM: N-(4-amino-5-(3-methoxypropoxy)pyridin-2-yl)acetamide (Preparation |
| | 262) |
| | Het: 4-chloro-2-(1,1-difluoroethyl)-6-ethylpyrimidine (Preparation 94) |
| | 148 mg, 54.0% yield as a white solid. Prep-HPLC (Method N, Gradient 22- |
| | 52%) |
| | LCMS m/z = 410.3 [M + H]⁺. ¹H NMR: (400 MHz, DMSO-d₆) δ ppm: 10.22 (s, |
| | 1H), 9.08 (s, 1H), 8.98 (s, 1H), 8.00 (s, 1H), 7.14 (s, 1H), 4.16 (t, J = 6.4 Hz, |
| | 2H), 3.48 (t, J = 6.4 Hz, 2H), 3.24 (s, 3H), 2.68 (q, J = 7.6 Hz, 2H), 1.98-2.12 |
| | (m, 8H), 1.23 (t, J = 7.6 Hz, 3H). |
| 70 | N-(4-((2-(1,1-difluoroethyl)-6-ethylpyrimidin-4-yl)amino)-5-(2- |
| | morpholinoethoxy)pyridin-2-yl)acetamide |
| | 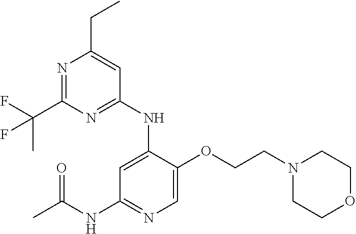 [SMILES: CCc1cc(Nc2cc(NC(C)=O)ncc2OCCN2CCOCC2)nc(C(C)(F)F)n1] |
| | SM: N-(4-amino-5-(2-morpholinoethoxy)pyridin-2-yl)acetamide (Preparation |
| | 263) |
| | Het: 4-chloro-2-(1,1-difluoroethyl)-6-ethylpyrimidine (Preparation 94) |
| | 20.4 mg, 12.7% yield as a white solid. |
| | Prep-HPLC (Method B, Gradient 25-55%) LCMS m/z = 451.1 [M + H]⁺. |
| | ¹H NMR: (500 MHz, CDCl₃) δ ppm: 8.91 (s, 1H), 8.71 (s, 1H), 7.99 (s, 1H), |
| | 7.92 (s, 1H), 6.94 (s, 1H), 4.24 (t, J = 5.5, 2H), 3.81-3.84 (m, 4H), 2.78-2.82 (m, |
| | 2H), 2.71-2.74 (m, 2H), 2.57-2.58 (m, 4H), 2.20 (s, 3H), 2.12 (t, J = 18.5, 3H), |
| | 1.33 (t, J = 7.5, 3H). |
| 71 | N-(4-((2-(1, 1-difluoroethyl)-6-ethylpyrimidin-4-yl)amino)-5-(2-(4- |
| | methylpiperazin-1-yl)ethoxy)pyridin-2-yl)acetamide |
| | 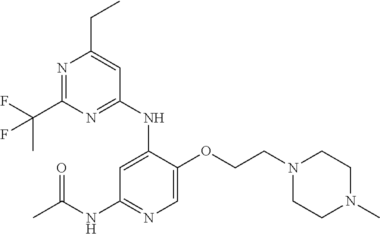 [SMILES: CCc1cc(Nc2cc(NC(C)=O)ncc2OCCN2CCN(C)CC2)nc(C(C)(F)F)n1] |
| | SM: N-(4-amino-5-(2-(4-methylpiperazin-1-yl)ethoxy)pyridin-2-yl)acetamide |
| | (Preparation 264) |
| | Het: 4-chloro-2-(1,1-difluoroethyl)-6-ethylpyrimidine (Preparation 94) |
| | 50.8 mg, 16.1% yield as a white solid. Prep-HPLC (Method B, Gradient 21- |
| | 50%) |
| | LCMS m/z = 464.2 [M + H]⁺. ¹H NMR: (500 MHz, CDCl₃) δ ppm: 8.95 (br s, |
| | 1H), 8.71 (br s, 1H), 7.91 (s, 1H), 7.78 (s, 1H), 6.94 (s, 1H), 4.24 (t, J = 5.0 Hz, |
| | 2H), 3.78-3.83 (m, 2H), 2.59-2.75 (m, 10H), 3.38 (s, 3H), 2.20 (s, 3H), 2.13 (t, |
| | J = 18.5 Hz, 3H), 1.33 (t, J = 8.0 Hz, 3H). |
| 72 | N-(5-(2-methoxyethoxy)-4-((6-(tetrahydrofuran-3-yl)pyridin-2- |
| | yl)amino)pyridin-2-yl)acetamide |
| | 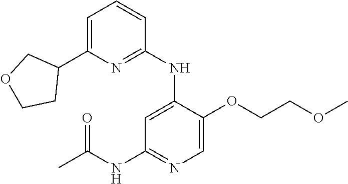 [SMILES: COCCOc1cnc(NC(C)=O)cc1Nc1cccc(C2CCOC2)n1] |
| | SM: N-(4-amino-5-(2-methoxyethoxy)pyridin-2-yl)acetamide (Preparation 63) |
| | Het: 2-bromo-6-(tetrahydrofuran-3-yl)pyridine |
| | 45.0 mg, 49.6% yield as a white solid. Prep-HPLC (Method N, Gradient 22- |
| | 52%) |
| | LCMS m/z = 373.2 [M + H]⁺. ¹H NMR: (400 MHz, CDCl₃) δ ppm: 9.10 (s, 1H), |
| | 7.99 (br s, 1H), 7.83 (s, 1H), 7.76 (s, 1H), 7.53 (t, J = 8.0 Hz, 1H), 6.78-6.81 (m, |
| | 2H), 4.18-4.22 (m, 3H), 4.08-4.15 (m, 1H), 3.95-3.97 (m, 2H), 3.74-3.76 (m, |
| | 2H), 3.52-3.60 (m, 1H), 3.49 (s, 3H), 2.36-2.41 (m, 2H), 2.17 (s, 3H). |
| 73 | N-(4-((6-(1,2-difluoroethyl)pyridin-2-yl)amino)-5-(2-methoxyethoxy)pyridin-2- |
| | yl)acetamide |
| | 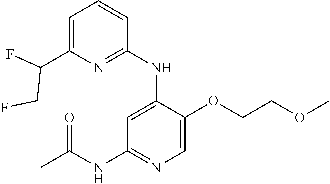 [SMILES: COCCOc1cnc(NC(C)=O)cc1Nc1cccc(C(F)CF)n1] |
| | SM: N-(4-amino-5-(2-methoxyethoxy)pyridin-2-yl)acetamide (Preparation 63) |
| | Het: 2-bromo-6-(1,2-difluoroethyl)pyridine (Preparation 185) |
| | 75 mg, 46.1% yield as a white solid. Prep-HPLC (Method O, Gradient 20-50%) |
| | LCMS m/z =367.1 [M + H]⁺. ¹H NMR: (400 MHz, CDCl₃) δ ppm: 9.31 (s, 1H), |
| | 7.85-7.89 (m, 3H), 7.64-7.68 (m, 1H), 7.12 (d, J = 7.6 Hz, 1H), 6.81 (d, J = 8.0 |
| | Hz, 1H), 5.75-5.87 (m, 1H), 5.02-5.21 (m, 2H), 4.18-4.20 (m, 2H), 3.74-3.76 |
| | (m, 2H), 3.50 (s, 3H), 2.18 (s, 3 H). |

### Example 74

### N-(5-((2,2-difluorocyclopropyl)methoxy)-4-((2-(1,1-difluoroethyl)-6-methylpyrimidin-4-yl)amino)pyridin-2-yl)acetamide

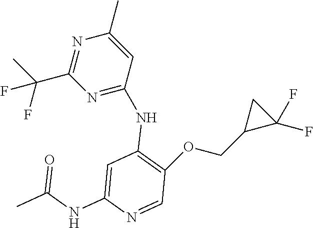 [SMILES: CC(=O)Nc1cc(Nc2cc(C)nc(C(C)(F)F)n2)c(OCC2CC2(F)F)cn1]

To a solution of N-(4-amino-5-((2,2-difluorocyclopropyl)methoxy)pyridin-2-yl)acetamide (Preparation 253, 200 mg, 0.778 mmol, HCl salt) in dioxane (5 mL) was added 4-chloro-2-(1,1-difluoroethyl)-6-methylpyrimidine (Preparation 92, 165 mg, 0.855 mmol), Xantphos (89.9 mg, 0.155 mmol), Cs₂CO₃ (507 mg, 1.55 mmol) and Pd₂(dba)₃ (71.2 mg, 77.8 umol). The mixture was stirred at 120° C. for 16 h under N₂, then concentrated. The crude product was purified by Prep-HPLC (Method C, gradient 28-55%) to give N-(5-((2,2-difluorocyclopropyl)methoxy)-4-((2-(1,1-difluoroethyl)-6-methylpyrimidin-4-yl)amino)pyridin-2-yl)acetamide (31 mg, 9.6500 yield) as a yellow solid. LCMS m/z=414.1 [M+H]⁺. ¹H NMR: (400 MHz, CDCl₃) δ ppm: 9.06 (s, 1H), 7.80-7.85 (m, 2H), 7.50 (s, 1H), 6.85 (s, 1H), 4.24-4.28 (m, 1H), 4.08-4.13 (m, 1H), 2.56 (s, 3H), 2.11-2.20 (m, 7H), 1.66-1.69 (m, 1H), 1.31-1.34 (m, 1H).

### Examples 75 to 82

The compounds in the following table were prepared from N-(4-amino-5-(2-methoxyethoxy)pyridin-2-yl)acetamide (Preparation 63) and the appropriate pyridine or pyrimidine (Het) following a similar procedure to that described in Example 74.

| Example | |
|---|---|
| No | Name, Structure, Het, Data |
| 75 | N-(4-((4-(1,1-difluoroethyl)pyrimidin-2-yl)amino)-5-(2- |
| | methoxyethoxy)pyridin-2-yl)acetamide |
| | 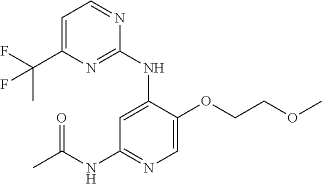 [SMILES: COCCOc1cnc(NC(C)=O)cc1Nc1nccc(C(C)(F)F)n1] |
| | Het: 2-chloro-4-(1,1-difluoroethyl)pyrimidine |
| | 60.0 mg, 18.4% yield as a white solid. Prep-HPLC (Method P, Gradient 25- |
| | 55%) |
| | LCMS m/z = 396.1 [M + H]⁺. ¹H NMR: (400 MHz, CDCl₃) δ ppm: 8.13 (s, 1H), |
| | 7.42 (d, J = 5.2 Hz, 1H), 7.22 (s, 1H), 6.90 (br s, 1H), 6.61 (s, 1H), 5.90 (d, J = 4.8 |
| | Hz, 1H), 2.96-2.98 (m, 2H), 2.49-2.52 (m, 2H), 2.21-2.24 (m, 3H), 0.93 (s, 3H), |
| | 0.87 (t, J = 19.2 Hz, 3H). |
| 76 | N-(4-((2-(1,1-difluoroethyl)-6-isopropoxypyrimidin-4-yl)amino)-5-(2- |
| | methoxyethoxy)pyridin-2-yl)acetamide |
| | 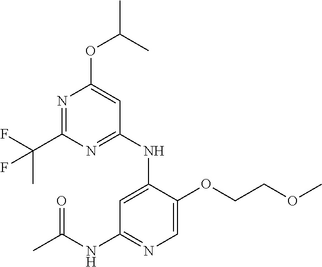 [SMILES: COCCOc1cnc(NC(C)=O)cc1Nc1cc(OC(C)C)nc(C(C)(F)F)n1] |
| | Het: 4-chloro-2-(1,1-difluoroethyl)-6-isopropoxypyrimidine (Preparation 162) |
| | 125 mg, 57.9% yield as a white solid. Prep-HPLC (Method Q, Gradient 34- |
| | 64%) |
| | LCMS m/z = 426.2 [M + H]⁺. ¹H NMR: (400 MHz, CDCl₃) δ ppm: 8.98 (br s, |
| | 1H), 8.11 (s, 1H), 8.08 (s, 1H), 7.87 (s, 1H), 6.25 (s, 1H), 5.37-5.47 (m, 1H), |
| | 4.17-4.20 (m, 2H), 3.72-3.75 (m, 2H), 3.50 (s, 3H), 2.20 (s, 3H), 2.10 (t, J = 18.8 |
| | Hz, 3H), 1.36 (d, J = 6.4 Hz, 6H). |
| 77 | N-(4-((2-(1, 1-difluoroethyl)-6-ethoxypyrimidin-4-yl)amino)-5-(2- |
| | methoxyethoxy)pyridin-2-yl)acetamide |
| | 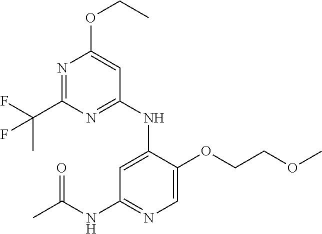 [SMILES: CCOc1cc(Nc2cc(NC(C)=O)ncc2OCCOC)nc(C(C)(F)F)n1] |
| | Het: 4-chloro-2-(1,1-difluoroethyl)-6-ethoxypyrimidine (Preparation 164) |
| | 38.5 mg, 20.8% yield as a white solid., Prep-HPLC (Method L, Gradient 36- |
| | 66%) |
| | LCMS m/z = 412.1 [M + H]⁺. ¹H NMR: (500 MHz, CDCl₃) δ ppm: 8.94 (s, 1H), |
| | 8.27 (s, 1H), 8.08 (s, 1H), 7.89 (s, 1H), 6.31 (s, 1H), 4.45 (q, J = 7.2, 2H), 4.16- |
| | 4.20 (m, 2H), 3.71-3.74 (m, 2H), 3.50 (s, 3H), 2.05-2.19 (m, 6H), 1.40 (t, J = |
| | 7.2, 3H). |
| 78 | 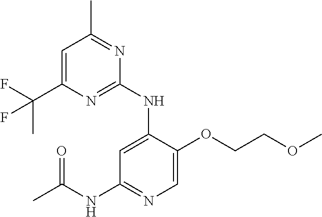 [SMILES: COCCOc1cnc(NC(C)=O)cc1Nc1nc(C)cc(C(C)(F)F)n1] |
| | Het: 2-chloro-4-(1,1-difluoroethyl)-6-methylpyrimidine (Preparation 133) |
| | 55 mg, 32.4% yield as a white solid. Prep-HPLC (Method B, Gradient 29-49%) |
| | LCMS m/z = 382.2 [M + H]⁺. ¹H NMR: (400 MHz, CDCl₃) δ ppm: 9.51 ( s, 1H), |
| | 8.44 (s, 1H), 8.13 ( s, 1H), 7.85 (s, 1H), 7.05 (s, 1H), 4.23 (d, J = 4.8 Hz, 2H), |
| | 3.77 (d, J = 4.4 Hz, 2H), 3.51 (s, 3H), 2.58 (s, 3H), 2.21 (s, 3H), 2.10 (t, J = 19.2 |
| | Hz, 3H). |
| 79 | N-(4-((2-(1,2-difluoroethyl)-6-methylpyrimidin-4-yl)amino)-5-(2- |
| | methoxyethoxy)pyridin-2-yl)acetamide |
| | 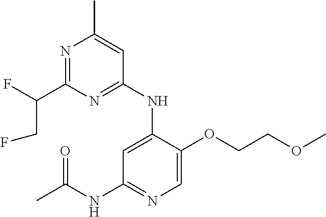 [SMILES: COCCOc1cnc(NC(C)=O)cc1Nc1cc(C)nc(C(F)CF)n1] |
| | Het: 4-chloro-2-(1,2-difluoroethyl)-6-methylpyrimidine (Preparation 195) |
| | 45.0 mg, 30.9% yield as a white solid. Prep-HPLC (Method B, Gradient15- |
| | 45%) |
| | LCMS m/z = 382.1 [M + H]⁺. ¹H NMR: (500 MHz, CDCl₃) δ ppm: 9.25 (s, 1H), |
| | 8.19 (s, 1H), 8.10 (s, 1H), 7.89 (s, 1H), 6.66 (s, 1H), 5.66-5.85 (m, 1H), 5.07- |
| | 5.19 (m, 2H), 4.17-4.20 (m, 2H), 3.73-3.75 (m, 2H), 3.50 (s., 3H), 2.50 (s, 3H), |
| | 2.20 (s, 3H). |
| 80 | N-(4-((4-(1,2-difluoroethyl)-6-methylpyrimidin-2-yl)amino)-5-(2- |
| | methoxyethoxy)pyridin-2-yl)acetamide |
| | 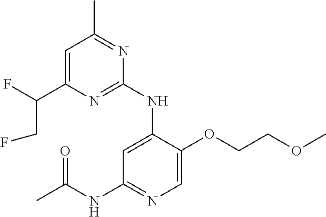 [SMILES: COCCOc1cnc(NC(C)=O)cc1Nc1nc(C)cc(C(F)CF)n1] |
| | Het: 2-chloro-4-(1,2-difluoroethyl)-6-methylpyrimidine (Preparation 191) |
| | 61.0 mg, 41.9% yield as a white solid. Prep-HPLC (Method B, Gradient 25- |
| | 45%) |
| | LCMS m/z = 382.1 [M + H]⁺. ¹H NMR: (500 MHz, CDCl₃) δ ppm: 9.50 (s, 1H), |
| | 8.32 (s, 1H), 8.24 (s, 1H), 7.84 (s, 1H), 6.99 (s, 1H), 5.59-5.74 (m, 1H), 5.03- |
| | 5.18 (m, 2H), 4.20-4.23 (m, 2H), 3.49-3.78 (m, 2H), 3.49 (s., 3H), 2.52 (s, 3H), |
| | 2.18 (s, 3H). |
| 81 | N-(4-((2-(1,2-difluoropropan-2-y1)-6-methylpyrimidin-4-yl)amino)-5-(2- |
| | methoxyethoxy)pyridin-2-yl)acetamide |
| | 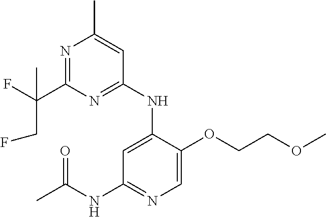 [SMILES: COCCOc1cnc(NC(C)=O)cc1Nc1cc(C)nc(C(C)(F)CF)n1] |
| | Het: 4-chloro-2-(1,2-difluoropropan-2-yl)-6-methylpyrimidine (Preparation |
| | 203) |
| | 70 mg, 40.6% yield as a yellow solid. Prep-HPLC (Method C, Gradient 20- |
| | 50%) |
| | LCMS m/z = 396.1 [M + H]⁺. ¹H NMR: (500 MHz, CDCl₃) δ ppm: 9.28 (s, 1H), |
| | 8.08 (s, 2H), 7.89 (s, 1H), 6.60 (s, 1H), 4.84-5.19 (m, 2H), 4.19-4.21 (m, 2H), |
| | 3.74-3.76 (m, 2H), 3.51 (s, 3H), 2.52 (s, 3H), 2.20 (s, 3H), 1.81-1.87 (m, 3H). |
| 82 | N-(4-((2-(1-fluoropropan-2-yl)-6-methylpyrimidin-4-yl)amino)-5-(2- |
| | methoxyethoxy)pyridin-2-yl)acetamide |
| | 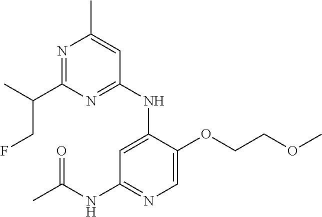 [SMILES: COCCOc1cnc(NC(C)=O)cc1Nc1cc(C)nc(C(C)CF)n1] |
| | Het: 4-chloro-2-(1-fluoropropan-2-yl)-6-methylpyrimidine (Preparation 200) |
| | 9.5 mg, 8.23% yield as a white solid. Prep-HPLC (Method B, Gradient 36-65%) |
| | LCMS m/z = 378.1 [M + H]⁺. ¹H NMR: (500 MHz, CDCl₃) δ ppm: 9.36 (br s, |
| | 1H), 7.99 (br s, 1H), 7.94 (s, 1H), 6.53 (s, 1H), 4.96-5.07 (m, 1H), 4.69-4.80 (m, |
| | 1H), 4.19-4.20 (m, 2H), 3.73-3.75 (m, 2H), 3.50 (s, 3H), 3.42-3.44 (m, 1H), |
| | 2.44 (s, 3H), 2.22 (s, 3H), 1.41 (d, J = 7.0 Hz, 3H). |

### Examples 83 to 88

The compounds in the following table were prepared from the corresponding ether substituted pyridines (SM) and the appropriate pyridine or pyrimidine (Het) following a similar procedure to that described in Example 74.

| Example | |
|---|---|
| No | Name, Structure, Starting Material (SM), Het, Data |
| 83 | N-(5-(1-cyclopropylethoxy)-4-((2-(1,1-difluoroethyl)-6-methylpyrimidin-4- |
| | yl)amino)pyridin-2-yl)acetamide |
| | 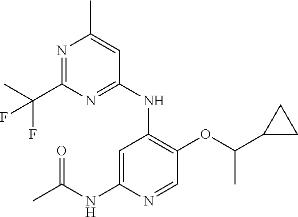 [SMILES: CC(=O)Nc1cc(Nc2cc(C)nc(C(C)(F)F)n2)c(OC(C)C2CC2)cn1] |
| | SM: N-(4-amino-5-(1-cyclopropylethoxy)pyridin-2-yl)acetamide (Preparartion |
| | 269) |
| | Het: 4-chloro-2-(1,1-difluoroethyl)-6-methylpyrimidine (Preparation 92) |
| | 16.5 mg, 11.4% yield as a white solid. Prep-HPLC (Method B, Gradient 31- |
| | 60%) |
| | LCMS m/z = 392.2 [M + H]⁺. ¹H NMR: (500 MHz, CDCl₃) δ ppm: 9.01 ( s, 1H), |
| | 8.09 (s, 1H), 7.84 (s, 1H), 7.63 (s, 1H), 6.84 (s, 1H), 3.69-3.81 (m, 1H), 2.54 |
| | (s, 3H), 2.04-2.30 (m, 6H), 1.44 (d, J = 6.0 Hz, 3H), 1.10-1.21 (m, 1H), 0.56-0.64 |
| | (m, 2H), 0.22-0.39 (m, 2H). |
| 84 | N-(5-((1-cyanocyclopropyl)methoxy)-4-((2-(1,1-difluoroethyl)-6- |
| | methylpyrimidin-4-yl)amino)pyridin-2-yl)acetamide |
| | 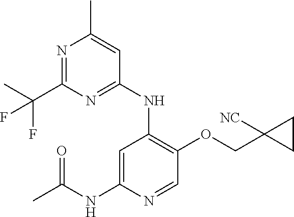 [SMILES: CC(=O)Nc1cc(Nc2cc(C)nc(C(C)(F)F)n2)c(OCC2(C#N)CC2)cn1] |
| | SM: N-(4-amino-5-((1-cyanocyclopropyl)methoxy)pyridin-2-yl)acetamide |
| | (Preparation 254) |
| | Het: 4-chloro-2-(1,1-difluoroethyl)-6-methylpyrimidine (Preparation 92) |
| | 25 mg, 12.8% yield as a yellow solid. Prep-HPLC (Method C, Gradient 20- |
| | 40%) |
| | LCMS m/z = 403.1 [M + H]⁺. ¹H NMR: (400 MHz, CDCl₃) δ ppm: 9.27 (s, 1H), |
| | 7.93 (s, 1H), 7.79 (s, 1H), 7.66 (s, 1H), 6.84 (s, 1H), 4.05 (s, 2H), 2.55 (s, 3H), |
| | 2.13-2.23 (m, 6H), 1.49-1.52 (m, 2H), 1.12-1.16 (m, 2H). |
| 85 | N-(4-((2-(1, 1-difluoroethyl)-6-methylpyrimidin-4-yl)amino)-5-(oxetan-2- |
| | ylmethoxy)pyridin-2-yl)acetamide |
| | 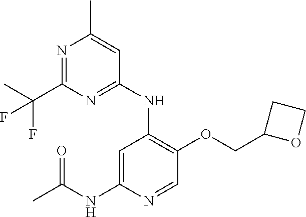 [SMILES: CC(=O)Nc1cc(Nc2cc(C)nc(C(C)(F)F)n2)c(OCC2CCO2)cn1] |
| | SM: N-(4-amino-5-(oxetan-2-ylmethoxy)pyridin-2-yl)acetamide (Preparation |
| | 267) |
| | Het: 4-chloro-2-(1,1-difluoroethyl)-6-methylpyrimidine (Preparation 92) |
| | 38.5 mg, 34.3% yield as a white solid. Prep-HPLC (Method K, Gradient 29- |
| | 59%) |
| | LCMS m/z = 394.1 [M + H]⁺. ¹H NMR: (400 MHz, CDCl₃) δ ppm: 9.26 (s, 1H), |
| | 8.51 (s, 1H), 7.96 (s, 1H), 7.92 (s, 1H), 6.68 (s, 1H), 5.17-5.19 (m, 1H), 4.84- |
| | 4.86 (m, 1H), 4.72-4.74 (m, 1H), 4.22-4.26 (m, 1H), 4.04-4.08 (m, 1H), 2.77- |
| | 2.83 (m, 2H), 2.52 (s, 3H), 2.13-2.23 (m, 6H). |
| 86 | N-(4-((2-(1,1-difluoroethyl)-6-methylpyrimidin-4-yl)amino)-5-(oxetan-3- |
| | yloxy)pyridin-2-yl)acetamide |
| | 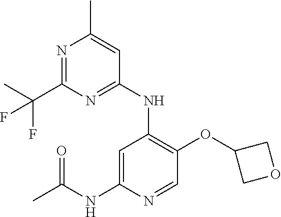 [SMILES: CC(=O)Nc1cc(Nc2cc(C)nc(C(C)(F)F)n2)c(OC2COC2)cn1] |
| | SM: N-(4-amino-5-(oxetan-3-yloxy)pyridin-2-yl)acetamide (Preparation 268) |
| | Het: 4-chloro-2-(1,1-difluoroethyl)-6-methylpyrimidine (Preparation 92) |
| | 12.6 mg, 14.8% yield as a white solid. Prep-HPLC (Method B, Gradient 15- |
| | 45%) |
| | LCMS m/z = 380.1 [M + H]⁺. ¹H NMR: (400 MHz, CDCl₃) δ ppm: 9.08 (s, 1H), |
| | 7.97 (s, 1H), 7.49 (s, 1H), 7.38 (s, 1H), 6.91 (s, 1H), 5.29-5.34 (m, 1H), 5.01- |
| | 5.05 (m, 2H), 4.80-4.84 (m, 2H), 2.57 (s, 3H), 2.10-2.22 (m, 6H). |
| 87 | methyl (4-((2-(1,1-difluoroethyl)-6-ethylpyrimidin-4-yl)amino)-5-(2- |
| | methoxyethoxy)pyridin-2-yl)carbamate |
| | 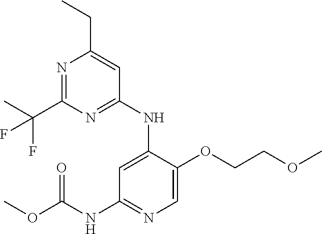 [SMILES: CCc1cc(Nc2cc(NC(=O)OC)ncc2OCCOC)nc(C(C)(F)F)n1] |
| | SM: methyl (4-amino-5-(2-methoxyethoxy)pyridin-2-yl)carbamate (Preparation |
| | 259) |
| | Het: 4-chloro-2-(1,1-difluoroethyl)-6-ethylpyrimidine (Preparation 94) |
| | 91 mg, 53.4% yield as a white solid. Prep-HPLC (Method B, Gradient 19-48%) |
| | LCMS m/z = 412.1 [M + H]⁺. ¹H NMR: (500 MHz, CDCl₃) δ ppm: 8.96 (s, 1H), |
| | 8.22 (s, 1H), 8.18 (s, 1H), 7.95 (s, 1H), 6.76 (s, 1H), 4.18-4.20 (s, 2H), 3.81 (s, |
| | 2H), 3.73-3.77 (m, 2H), 3.51 (s, 3H), 2.77-2.87 (m, 2H), 2.15 (t, J=18.8 Hz, |
| | 3H), 1.33 (t, J-7.6 Hz, 3H). |
| 88 | N-(4-((2-(1,1-difluoroethyl)-6-ethylpyrimidin-4-yl)amino)-5-(2- |
| | methoxyethoxy)pyridin-2-yl)propionamide |
| | 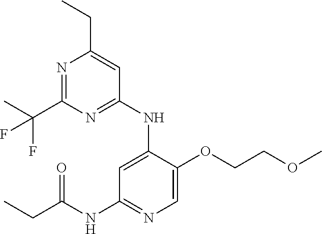 [SMILES: CCC(=O)Nc1cc(Nc2cc(CC)nc(C(C)(F)F)n2)c(OCCOC)cn1] |
| | SM: N-(4-amino-5-(2-methoxyethoxy)pyridin-2-yl)propionamide (Preparation |
| | 260) |
| | Het: 4-chloro-2-(1,1-difluoroethyl)-6-ethylpyrimidine (Preparation 94) |
| | 45 mg, 26.3% yield as a white solid. Prep-HPLC (Method B, Gradient 19-48%) |
| | LCMS m/z = 410.1 [M + H]⁺. ¹H NMR: (500 MHz, CDCl₃) δ ppm: 9.15 (s, 1H), |
| | 8.18 (s, 1H), 7.97 (s, 1H), 7.79 (s, 1H), 6.80 (s, 1H), 4.18-4.21 (m, 1H), 3.74- |
| | 3.76 (m, 1H), 3.51 (s, 3H), 2.79-3.87 (m, 2H), 2.38-2.46 (m, 2H), 2.11-2.22 (m, |
| | 3H), 1.23-1.36 (m, 6H). |

### Example 89 and 90

### N-(4-((6-(1,2-difluoroethyl)pyrazin-2-yl)amino)-5-(2-methoxyethoxy)pyridin-2-yl)acetamide and N-(4-((6-(1-fluorovinyl)pyrazin-2-yl)amino)-5-(2-methoxyethoxy)pyridin-2-yl)acetamide

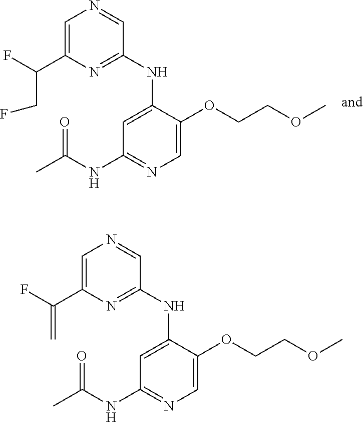 [SMILES: COCCOc1cnc(NC(C)=O)cc1Nc1cncc(C(F)CF)n1 ; C=C(F)c1cncc(Nc2cc(NC(C)=O)ncc2OCCOC)n1]

To a solution of 2-bromo-6-(1,2-difluoroethyl)pyrazine (Preparation 188, 80.0 mg, 0.355 mmol), N-(4-amino-5-(2-methoxyethoxy)pyridin-2-yl)acetamide (Preparation 63, 79.2 mg, 0.355 mmol) and K₃PO₄ (377 mg, 1.78 mmol) in dioxane (2.0 mL) were added Xantphos (20.6 mg, 35.5 umol) and Pd₂(dba)₃ (32.6 mg, 35.5 umol). The reaction mixture was stirred at 70° C. for 1 h, then purified by prep-HPLC (Method B, Gradient 17-47%) to give N-(4-((6-(1,2-difluoroethyl)pyrazin-2-yl)amino)-5-(2-methoxyethoxy)pyridin-2-yl)acetamide (17.0 mg, 13.0% yield) as a white solid: LCMS m/z=368.1 [M+H]⁺, ¹H NM/R: (500 MHz, CDCl₃) δ ppm: 9.41 (s, 1H), 8.37-8.34 (m, 3H), 7.90-7.98 (m, 2H), 5.81-5.91 (m, 1H), 5.09-5.25 (in, 2H), 4.18-4.22 (m, 2H), 3.75-3.78 (m, 2H), 3.54 (s, 3H), 2.20 (s, 3H), and N-(4-((6-(1-fluorovinyl)pyrazin-2-yl)amino)-5-(2-methoxyethoxy)pyridin-2-yl)acetamide (26.0 mg, 21.1% yield) as a white solid. LCMS m/z=348.1 [M+H]⁺, ¹H NMR: (500 MHz, CDCl₃) δ ppm: 9.53 (s, 1H), 8.71 (br s, 1H), 8.36-8.39 (m, 2H), 8.24 (s, 1H), 7.87 (s, 1H), 6.34-6.46 (m, 1H), 5.24-5.29 (m, 1H), 4.20-4.22 (m, 2H), 3.76-7.79 (m, 2H), 3.54 (s, 3H), 2.24 (s, 3H).

### Example 91

### N-(4-((6-cyclopropoxy-2-(1,1-difluoroethyl)pyrimidin-4-yl)amino)-5-methoxypyridin-2-yl)acetamide

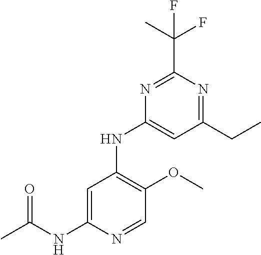 [SMILES: CCc1cc(Nc2cc(NC(C)=O)ncc2OC)nc(C(C)(F)F)n1]

To a flask containing N-(4-amino-5-methoxypyridin-2-yl)acetamide hydrochloride (Preparation 61, 20 g, 78.7 mmol) in dioxane (500 mL) were added 4-chloro-6-cyclopropoxy-2-(1,1-difluoroethyl)pyrimidine (Preparation 163, 20.3 g, 86.6 mmol), Cs₂CO₃ (76.9 g, 236 mmol) and DIPEA (12.2 g, 94.5 mmol). The reaction mixture was degassed, then backfilled with N₂, then heated to 95° C. After 10 min, a degassed solution of BINAP (4.90 g, 7.87 mmol) and Pd(OAc)₂ (884 mg, 3.94 mmol) in dioxane (100 mL) was heated to 95° C., then transferred via syringe. The reaction mixture was stirred at 95° C. for 5 h under N₂, then quenched with water (500 mL) and extracted with EtOAc (3×500 mL). The combined organic phases were washed with brine (1000 mL), dried over Na₂SO₄, filtered and concentrated under reduced pressure. The residue was purified by column chromatography on silica gel (15-75% 3:1 EtOAc:EtOH in heptane). The desired fractions were concentrated under reduced pressure and the residue azeotroped with EtOH. to afford a sticky yellow-white foam that was diluted with EtOH. The resulting solid was sonicated in EtOH, the solid filtered and dried to give N-(4-((6-cyclopropoxy-2-(1,1-difluoroethyl)pyrimidin-4-yl)amino)-5-methoxypyridin-2-yl)acetamide as a white solid, 12.8 g, 42.7%. LCMS m/z=380.0 [M+H]⁺. ¹H NMR: (400 MHz, DMSO-d₆) δ ppm: 14.55 (br s, 1H), 7.99-8.05 (m, 2H), 7.68-7.61 (m, 1H), 7.59-7.66 (m, 3H), 7.48 (s, 1H), 7.39 (d, J=2.01 Hz, 1H), 6.94 (dd, J=2.4, 8.8 Hz, 1H), 3.84 (s, 3H).

### Example 92

### methyl (4-((2-(1,1-difluoroethyl)-6-ethylpyrimidin-4-yl)amino)-5-(2-(dimethylamino)ethoxy)pyridin-2-yl)carbamate

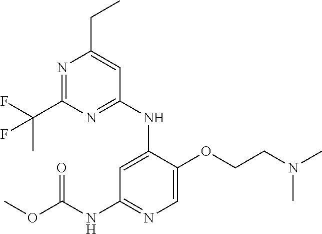

A mixture of Cs₂CO₃ (225 mg, 0.691 mmol), methyl (4-amino-5-(2-(dimethylamino)ethoxy)pyridin-2-yl)carbamate hydrochloride (Preparation 265, 75 mg, 0.229 mmol), DIPEA (89.04 mg, 0.689 mmol), 4-chloro-2-(1,1-difluoroethyl)-6-ethylpyrimidine (75 mg, 0.363 mmol), Pd(OAc)₂ (3 mg, 13.4 umol) and BINAP (10 mg, 16.06 umol) in dioxane (5 mL) was stirred at 90° C. for 2 h. The reaction mixture was cooled to rt, passed through a 0.2 um syringe filter, and rinsed with EtOAc (8 mL). The filtrate was concentrated to dryness and purified via prep-HPLC (Method U, Gradient 5-95%) to give methyl (4-((2-(1,1-difluoroethyl)-6-ethylpyrimidin-4-yl)amino)-5-(2-(dimethylamino)ethoxy)pyridin-2-yl)carbamate (44.8 mg, 46.1% yield). LCMS m/z=425.2 [M+H]⁺. ¹H NMR (600 MHz, DMSO-d₆) δ (ppm)=9.90 (s, 1H), 9.86 (s, 1H), 8.77 (s, 1H), 8.05 (s, 1H), 6.95 (s, 1H), 4.17 (t, J=5.5 Hz, 2H), 3.65 (s, 3H), 2.72 (q, J=7.4 Hz, 2H), 2.60 (t, J=5.5 Hz, 2H), 2.27 (s, 6H), 2.07 (t, J=19.1 Hz, 3H), 1.24 (t, J=7.4 Hz, 3H).

### Example 93

### N-(4-((6-cyclopropoxy-2-(1,1-difluoroethyl)pyrimidin-4-yl)amino)-5-(trifluoromethoxy)pyridin-2-yl)acetamide

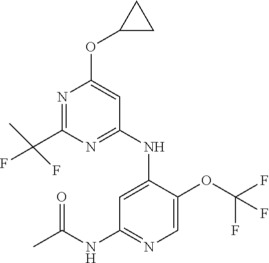 [SMILES: CC(=O)Nc1cc(Nc2cc(OC3CC3)nc(C(C)(F)F)n2)c(OC(F)(F)F)cn1]

A vial containing N-(2-chloro-5-(trifluoromethoxy)pyridin-4-yl)-6-cyclopropoxy-2-(1,1-difluoroethyl)pyrimidin-4-amine (Preparation 274, 125 mg, 0.302 mmol), acetamide (75 mg, 1.3 mmol), BrettPhos-Pd-G3 (32 mg, 35 umol) and Cs₂CO₃ (308.4 mg, 0.947 mmol) in dioxane (2 mL) was degassed, backfilled with N₂ then heated to 95° C. After 1.5 h, the mixture was cooled to rt then filtered through Celite®, rinsing through with 3:1 EtOAc:EtOH then the combined organic layers were concentrated under reduced pressure. The residue was loaded onto a silica gel column and purified with (20-75% EtOAc in heptane.) The product was further purified by HPLC (Method U, Gradient 5-75%) to give N-(4-((6-cyclopropoxy-2-(1,1-difluoroethyl)pyrimidin-4-yl)amino)-5-(trifluoromethoxy)pyridin-2-yl)acetamide as a white solid (15 mg, 11% yield). LCMS m/z=433.9 [M+H]⁺. ¹H NMR (500 MHz, DMSO-d₆) δ (ppm)=10.57 (br s, 1H), 9.87 (s, 1H), 9.14 (s, 1H), 8.30 (s, 1H), 6.91 (s, 1H), 4.19 (br s, 1H), 2.09-2.00 (m, 6H), 0.84 (br d, J=5.8 Hz, 2H), 0.81-0.77 (m, 2H).

### Example 94

### N-(4-((2-(1,1-difluoroethyl)-6-fluoropyrimidin-4-yl)amino)-5-ethoxypyridin-2-yl)acetamide trifluoroacetate

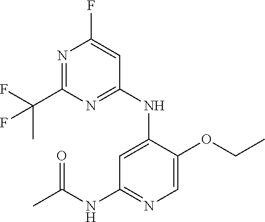 [SMILES: CCOc1cnc(NC(C)=O)cc1Nc1cc(F)nc(C(C)(F)F)n1]

TBAF (148 μL, 148 mol, 1 M in THF) was added to a solution of tert-butyl (2-acetamido-5-ethoxypyridin-4-yl)(6-chloro-2-(1,1-difluoroethyl)pyrimidin-4-yl)carbamate (Preparation 277, 35 mg, 74 mol) in DMF (2.5 mL) and the reaction was heated at 140° C. for 1 h. After cooling to rt, the reaction was diluted with saturated NaHCO₃, extracted twice with EtOAc and evaporated to dryness. The residue was purified by reverse phase HPLC (Method V, Gradient 5-45%), to give N-(4-((2-(1,1-difluoroethyl)-6-fluoropyrimidin-4-yl)amino)-5-ethoxypyridin-2-yl)acetamide trifluoroacetate (4 mg, 11% yield) as white solid. LCMS m/z=356.2 [M+H]⁺. ¹H NMR (600 MHz, DMSO-d₆) δ ppm 10.35 (br s, 1H), 9.66 (s, 1H), 8.88 (br s, 1H), 8.05 (s, 1H), 6.97 (s, 1H), 4.21 (m, 2H), 2.02-2.11 (m, 6H), 1.37 (t, J=6.9 Hz, 3H).

### Example 95

### N-(4-((6-(2-oxabicyclo[2.1.1]hexan-4-yl)-4-methylpyridin-2-yl)amino)-5-ethoxypyridin-2-yl)acetamide

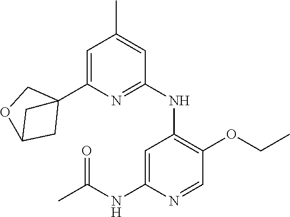 [SMILES: CCOc1cnc(NC(C)=O)cc1Nc1cc(C)cc(C23COC(C2)C3)n1]

To a vial equipped with magnetic stir bar was added NaOAc (359 mg, 4.38 mmol) and placed in a vacuum oven at 75° C. overnight. Separately, to another vial was added Cu(TMHD)₂ (117.76 mg, 0.274 mmol), 1,3-dioxoisoindolin-2-yl 2-oxabicyclo[2.1.1]hexane-4-carboxylate (Preparation 181, 224.4 mg, 0.821 mmol), aminosupersilane (435.8 mg, 1.10 mmol), bis[2-(2-pyridyl)phenyl]iridium(1+); 4-tert-butyl-2-(4-tert-butyl-2-pyridyl)pyridine; hexafluorophosphate (5.0 mg, 5.48 umol), N-(4-((6-bromo-4-methylpyridin-2-yl)amino)-5-ethoxypyridin-2-yl)acetamide (Preparation 275, 200 mg, 0.548 mmol) and dry acetone (8 mL). The resulting mixture was degassed with N₂, then transferred to the initial oven-dried vial containing base. The mixture was purged with N₂, sealed, then sonicated for 1 min. The reaction was placed in Integrated Photoreactor (450 nm LEDs) and stirred at rt for 2 h. The mixture was filtered and the filtrate was concentrated in vacuo. The residue was diluted with EtOAc, washed with water, then brine, dried and concentrated. The crude was purified by HPLC to give the title compound (20 mg, 7% yield) as a TFA salt. ¹H NMR (DMSO-d₆, 600 MHz) δ 8.8-9.1 (m, 1H), 8.5-8.8 (m, 1H), 7.83 (s, 1H), 7.1-7.2 (m, 1H), 6.91 (s, 1H), 4.57 (t, 1H, J=1.0 Hz), 4.19 (q, 2H, J=7.0 Hz), 3.92 (s, 2H), 2.30 (s, 3H), 2.2-2.2 (m, 2H), 2.14 (s, 3H), 1.8-1.9 (m, 2H), 1.43 (t, 3H, J=7.0 Hz). LCMS m/z=369 [M+H]+

### Example 96

### N-(4-((2-(1,1-difluoroethyl)-6-(pyridin-3-yl)pyrimidin-4-yl)amino)-5-ethoxypyridin-2-yl)acetamide

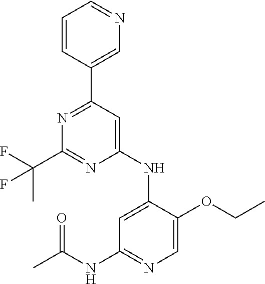 [SMILES: CCOc1cnc(NC(C)=O)cc1Nc1cc(-c2cccnc2)nc(C(C)(F)F)n1]

A mixture of tert-butyl (2-acetamido-5-ethoxypyridin-4-yl)(6-chloro-2-(1,1-difluoroethyl)pyrimidin-4-yl)carbamate (Preparation 277, 50 mg, 0.105 mmol), 3-(4,4,5,5-tetramethyl-1,3,2-dioxaborolan-2-yl)pyridine (60 mg, 0.29 mmol), K₂CO₃ (50 mg, 0.36 mmol), and Pd(dppf)Cl₂ (8 mg, 11 umol) in dioxane (2 mL) and water (1 mL) was stirred at 100° C. for 20 h. The reaction was cooled to rt, diluted with water (3 mL) and extracted with EtOAc (3×3 mL). The combined organic layers were concentrated to dryness and purified by silica gel chromatography (heptane to EtOAc with 2% dimethylethylamine) to give N-(4-((2-(1,1-difluoroethyl)-6-(pyridin-3-yl)pyrimidin-4-yl)amino)-5-ethoxypyridin-2-yl)acetamide as a white solid, (32 mg, 73% yield). LCMS m/z=415.2 [M+H]+. 1H NMR (600 MHz, DMSO-d₆) δ (ppm)=10.26 (s, 1H), 9.29 (s, 1H), 9.26 (dd, J=0.8, 2.3 Hz, 1H), 9.16-8.99 (m, 1H), 8.75 (dd, J=1.7, 4.8 Hz, 1H), 8.44 (d, J=8.4 Hz, 1H), 8.06 (s, 1H), 7.93 (s, 1H), 7.67-7.57 (m, 1H), 4.24 (q, J=6.9 Hz, 2H), 2.16 (t, J=19.3 Hz, 3H), 2.08 (s, 3H), 1.41 (t, J=7.1 Hz, 3H).

### Example 97

### N-(4-((2-(1,1-difluoroethyl)-6-(1-ethyl-1H-pyrazol-4-yl)pyrimidin-4-yl)amino)-5-methoxypyridin-2-yl)acetamide

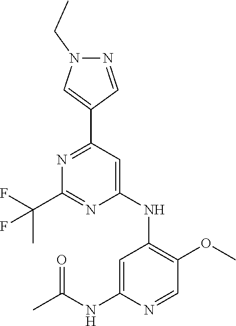 [SMILES: CCn1cc(-c2cc(Nc3cc(NC(C)=O)ncc3OC)nc(C(C)(F)F)n2)cn1]

A mixture of tert-butyl (2-acetamido-5-methoxypyridin-4-yl)(6-chloro-2-(1,1-difluoroethyl)pyrimidin-4-yl)carbamate (Preparation 278, 27 mg, 60 umol), (1-ethyl-4-(4,4,5,5-tetramethyl-1,3,2-dioxaborolan-2-yl)-1H-pyrazole (20 mg, 90 umol), Pd(dppf)Cl₂:DCM (4.9 mg, 6 umol) and Cs₂CO₃ (58 mg, 0.180 mmol) in dioxane (1 mL) and water (0.2 ml) was stirred at 90° C. for 3 h. The reaction was concentrated in vacuo and a solution of TFA (10 uL, 0.120 mmol) and DCM (2 mL) was added to the residue and the mixture stirred for 16 h. The reaction was evaporated and purified by (Method U, Gradient 5-95%) to afford N-(4-((2-(1,1-difluoroethyl)-6-(1-ethyl-1H-pyrazol-4-yl)pyrimidin-4-yl)amino)-5-methoxypyridin-2-yl)acetamide (3.7 mg, 14%) as a yellow solid. LCMS m/z=418 [M+H]⁺

### Example 98

### N-(4-((2-(1,1-difluoroethyl)-6-methylpyrimidin-4-yl)amino)-5-((6-methylpyrazin-2-yl)oxy)pyridin-2-yl)acetamide

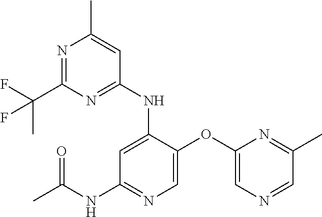 [SMILES: CC(=O)Nc1cc(Nc2cc(C)nc(C(C)(F)F)n2)c(Oc2cncc(C)n2)cn1]

A mixture of N-(4-((2-(1,1-difluoroethyl)-6-methylpyrimidin-4-yl)amino)-5-hydroxypyridin-2-yl)acetamide (Preparation 279, 25 mg, 0.077 mmol), 2-chloro-6-methyl-pyrazine (20 mg, 0.155 mmol), DIPEA (20 mg, 0.155 mmol), and MeCN (2.0 mL) was stirred for 72 h at 90° C. The reaction was cooled to rt, concentrated to dryness, and purified via prep-HPLC (Method U, Gradient 5-95%) to give N-(4-((2-(1,1-difluoroethyl)-6-methylpyrimidin-4-yl)amino)-5-((6-methylpyrazin-2-yl)oxy)pyridin-2-yl)acetamide (4.0 mg, 19% yield). LCMS m/z=416.2 [M+H]+.

### Examples 99 to 137

To a solution of N-(4-((2-(1,1-difluoroethyl)-6-methylpyrimidin-4-yl)amino)-5-hydroxypyridin-2-yl)acetamide (Preparation 279, 25.8 mg, 0.08 mmol) and the appropriate alcohol (0.08 mmol) in toluene (1.00 mL) was added (cyanomethylene)tributylphosphorane (77.1 mg, 0.32 mmol) under N₂. The reaction mixture was shaken at 50° C. for 16 h under N₂. The reaction mixtures were concentrated and the residues were purified by preparative HPLC to give the desired products.

Basic prep. HPLC conditions: Column: Xtimate C18 150*25 mm*5 μm; Mobile phase: A: NH₄OH/H₂O=0.05% v/v; B: CAN; Flow rate: 25 mL/min or 30 mL/min; Acidic prep. HPLC conditions: Column: Xtimate C18 150*25 mm*5 μm; Mobile phase: A: FA/H₂O=0.225% v/v; B: CAN; Flow rate: 25 mL/min or 30 mL/min;

| | | LCMS |
|---|---|---|
| Example | | m/z |
| No | Name, Structure, ROH, Data | [M + H]⁺ |
| 99 | N-(4-((2-(1,1-difluoroethyl)-6-methylpyrimidin-4-yl)amino)-5- | 396 |
| | ((1-fluorocyclopropyl)methoxy)pyridin-2-yl)acetamide | |
| | 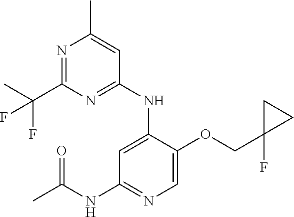 [SMILES: CC(=O)Nc1cc(Nc2cc(C)nc(C(C)(F)F)n2)c(OCC2(F)CC2)cn1] | |
| | ROH: (1-fluorocyclopropyl)methanol | |
| 100 | N-(5-(bicyclo[1.1.1]pentan-1-ylmethoxy)-4-((2-(1,1- | 404 |
| | difluoroethyl)-6-methylpyrimidin-4-yl)amino)pyridin-2- | |
| | yl)acetamide | |
| | 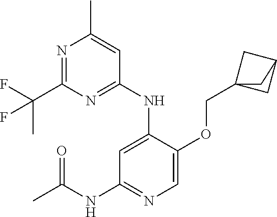 [SMILES: CC(=O)Nc1cc(Nc2cc(C)nc(C(C)(F)F)n2)c(OCC23CC(C2)C3)cn1] | |
| | ROH: bicyclo[1.1.1]pentan-1-ylmethanol | |
| 101 | N-(4-((2-(1,1-difluoroethyl)-6-methylpyrimidin-4-yl)amino)-5- | 415 |
| | (pyridin-2-ylmethoxy)pyridin-2-yl)acetamide | |
| | 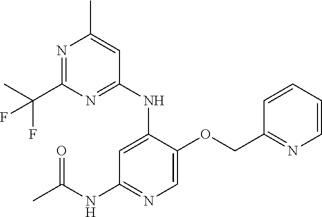 [SMILES: CC(=O)Nc1cc(Nc2cc(C)nc(C(C)(F)F)n2)c(OCc2ccccn2)cn1] | |
| | ROH: pyridin-2-ylmethanol | |
| 102 | 2-((6-acetamido-4-((2-(1,1-difluoroethyl)-6-methylpyrimidin-4- | 395 |
| | yl)amino)pyridin-3-yl)oxy)-N-methylacetamide | |
| | 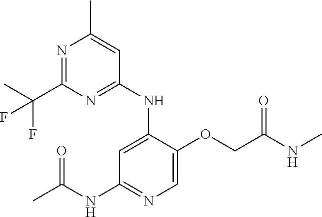 [SMILES: CNC(=O)COc1cnc(NC(C)=O)cc1Nc1cc(C)nc(C(C)(F)F)n1] | |
| | ROH: 2-hydroxy-N-methylacetamide | |
| 103 | 2-((6-acetamido-4-((2-(1,1-difluoroethyl)-6-methylpyrimidin-4- | 409 |
| | yl)amino)pyridin-3-yl)oxy)-N,N-dimethylacetamide | |
| | 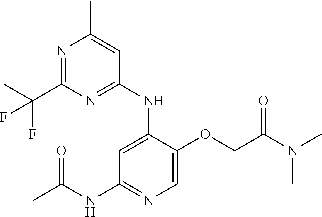 [SMILES: CC(=O)Nc1cc(Nc2cc(C)nc(C(C)(F)F)n2)c(OCC(=O)N(C)C)cn1] | |
| | ROH: 2-hydroxy-N,N-dimethylacetamide | |
| 104 | N-(4-((2-(1,1-difluoroethyl)-6-methylpyrimidin-4-yl)amino)-5- | 402 |
| | (3,3-difluoropropoxy)pyridin-2-yl)acetamide | |
| | 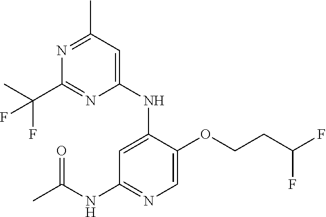 [SMILES: CC(=O)Nc1cc(Nc2cc(C)nc(C(C)(F)F)n2)c(OCCC(F)F)cn1] | |
| | ROH: 3,3-difluoropropanol | |
| 105 | N-(5-butoxy-4-((2-(1,1-difluoroethyl)-6-methylpyrimidin-4- | 380 |
| | yl)amino)pyridin-2-yl)acetamide | |
| | 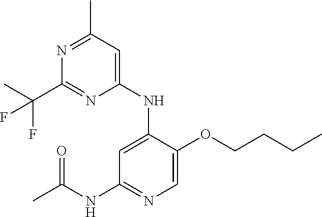 [SMILES: CCCCOc1cnc(NC(C)=O)cc1Nc1cc(C)nc(C(C)(F)F)n1] | |
| | ROH: butan-1-ol | |
| 106 | N-(4-((2-(1,1-difluoroethyl)-6-methylpyrimidin-4-yl)amino)-5-(2- | 450 |
| | (4-methylpiperazin-1-yl)ethoxy)pyridin-2-yl)acetamide | |
| | 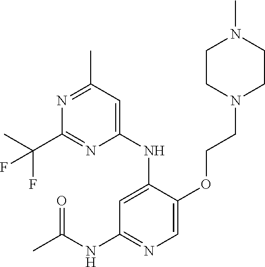 [SMILES: CC(=O)Nc1cc(Nc2cc(C)nc(C(C)(F)F)n2)c(OCCN2CCN(C)CC2)cn1] | |
| | ROH: (4-methylpiperazin-1-yl)ethanol | |
| 107 | N-(4-((2-(1,1-difluoroethyl)-6-methylpyrimidin-4-yl)amino)-5-(3- | 396 |
| | methoxypropoxy)pyridin-2-yl)acetamide | |
| | 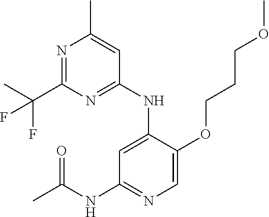 [SMILES: COCCCOc1cnc(NC(C)=O)cc1Nc1cc(C)nc(C(C)(F)F)n1] | |
| | ROH: 3-methoxypropanol | |
| 108 | N-(4-((2-(1,1-difluoroethyl)-6-methylpyrimidin-4-yl)amino)-5- | 421 |
| | (thiazol-2-ylmethoxy)pyridin-2-yl)acetamide | |
| | 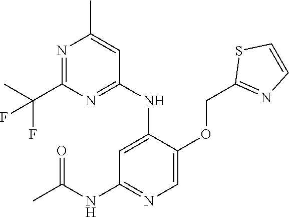 [SMILES: CC(=O)Nc1cc(Nc2cc(C)nc(C(C)(F)F)n2)c(OCc2nccs2)cn1] | |
| | ROH: thiazol-2-ylmethanol | |
| 109 | N-(4-((2-(1,1-difluoroethyl)-6-methylpyrimidin-4-yl)amino)-5- | 422 |
| | ((3-ethyloxetan-3-yl)methoxy)pyridin-2-yl)acetamide | |
| | 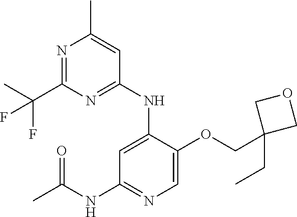 [SMILES: CCC1(COc2cnc(NC(C)=O)cc2Nc2cc(C)nc(C(C)(F)F)n2)COC1] | |
| | ROH: (3-ethyloxetan-3-yl)methanol | |
| 110 | (R)-N-(4-((2-(1,1-difluoroethyl)-6-methylpyrimidin-4-yl)amino)- | 438 |
| | 5-((2,2-dimethyl-1,3-dioxolan-4-yl)methoxy)pyridin-2- | |
| | yl)acetamide | |
| | 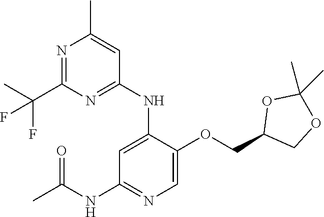 [SMILES: CC(=O)Nc1cc(Nc2cc(C)nc(C(C)(F)F)n2)c(OC[C@@H]2COC(C)(C)O2)cn1] | |
| | ROH: (R)-(2,2-dimethyl-1,3-dioxolan-4-yl)methanol | |
| 111 | N-(4-((2-(1,1-difluoroethyl)-6-methylpyrimidin-4-yl)amino)-5- | 435 |
| | ((3-methylisothiazol-4-yl)methoxy)pyridin-2-yl)acetamide | |
| | 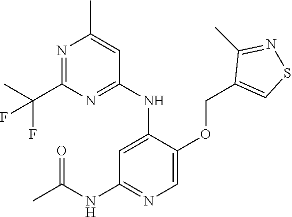 [SMILES: CC(=O)Nc1cc(Nc2cc(C)nc(C(C)(F)F)n2)c(OCc2csnc2C)cn1] | |
| | ROH: (3-methylisothiazol-4-yl)methanol | |
| 112 | N-(4-((2-(1,1-difluoroethyl)-6-methylpyrimidin-4-yl)amino)-5- | 435 |
| | ((4-methylthiazol-2-yl)methoxy)pyridin-2-yl)acetamide | |
| | 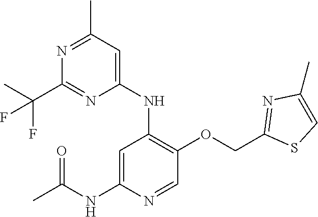 [SMILES: CC(=O)Nc1cc(Nc2cc(C)nc(C(C)(F)F)n2)c(OCc2nc(C)cs2)cn1] | |
| | ROH: (4-methylthiazol-2-yl)methanol | |
| 113 | N-(5-(2-(tert-butoxy)ethoxy)-4-((2-(1,1-difluoroethyl)-6- | 424 |
| | methylpyrimidin-4-yl)amino)pyridin-2-yl)acetamide | |
| | 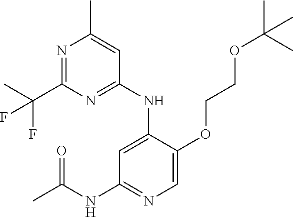 [SMILES: CC(=O)Nc1cc(Nc2cc(C)nc(C(C)(F)F)n2)c(OCCOC(C)(C)C)cn1] | |
| | ROH: 2-(tert-butoxy)ethanol | |
| 114 | N-(4-((2-(1,1-difluoroethyl)-6-methylpyrimidin-4-yl)amino)-5- | 445 |
| | ((2-methoxypyridin-4-yl)methoxy)pyridin-2-yl)acetamide | |
| | 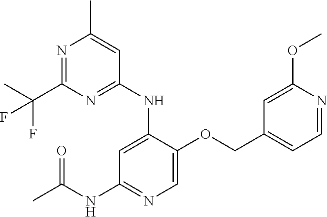 [SMILES: COc1cc(COc2cnc(NC(C)=O)cc2Nc2cc(C)nc(C(C)(F)F)n2)ccn1] | |
| | ROH: (2-methoxypyridin-4-yl)methanol | |
| 115 | N-(4-((2-(1,1-difluoroethyl)-6-methylpyrimidin-4-yl)amino)-5- | 435 |
| | ((5-methylisothiazol-3-yl)methoxy)pyridin-2-yl)acetamide | |
| | 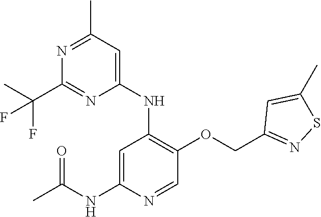 [SMILES: CC(=O)Nc1cc(Nc2cc(C)nc(C(C)(F)F)n2)c(OCc2cc(C)sn2)cn1] | |
| | ROH: (5-methylisothiazol-3-yl)methanol | |
| 116 | N-(4-((2-(1,1-difluoroethyl)-6-methylpyrimidin-4-yl)amino)-5- | 429 |
| | ((6-methylpyridin-3-yl)methoxy)pyridin-2-yl)acetamide | |
| | 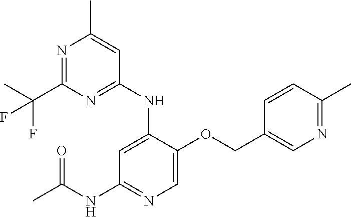 [SMILES: CC(=O)Nc1cc(Nc2cc(C)nc(C(C)(F)F)n2)c(OCc2ccc(C)nc2)cn1] | |
| | ROH: (6-methylpyridin-3-yl)methanol | |
| 117 | N-(4-((2-(1,1-difluoroethyl)-6-methylpyrimidin-4-yl)amino)-5- | 435 |
| | ((2-methylthiazol-4-yl)methoxy)pyridin-2-yl)acetamide | |
| | 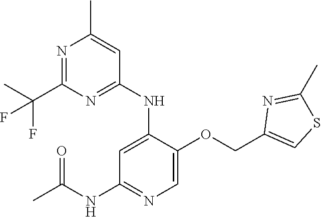 [SMILES: CC(=O)Nc1cc(Nc2cc(C)nc(C(C)(F)F)n2)c(OCc2csc(C)n2)cn1] | |
| | ROH: (2-methylthiazol-4-yl)methanol | |
| 118 | N-(4-((2-(1,1-difluoroethyl)-6-methylpyrimidin-4-yl)amino)-5- | 436 |
| | ((5-methyl-1,3,4-thiadiazol-2-yl)methoxy)pyridin-2-yl)acetamide | |
| | 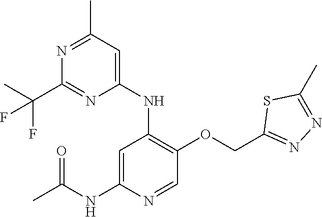 [SMILES: CC(=O)Nc1cc(Nc2cc(C)nc(C(C)(F)F)n2)c(OCc2nnc(C)s2)cn1] | |
| | ROH: (5-methyl-1,3,4-thiadiazol-2-yl)methanol | |
| 119 | N-(4-((2-(1, 1-difluoroethyl)-6-methylpyrimidin-4-yl)amino)-5-(2- | 398 |
| | fluoro-2-methylpropoxy)pyridin-2-yl)acetamide | |
| | 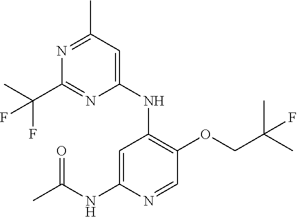 [SMILES: CC(=O)Nc1cc(Nc2cc(C)nc(C(C)(F)F)n2)c(OCC(C)(C)F)cn1] | |
| | ROH: 2-fluoro-2-methylpropanol | |
| 120 | N-(4-((2-(1,1-difluoroethyl)-6-methylpyrimidin-4-yl)amino)-5- | 433 |
| | ((2-fluoropyridin-4-yl)methoxy)pyridin-2-yl)acetamide | |
| | 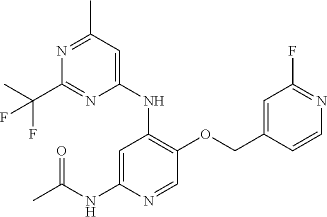 [SMILES: CC(=O)Nc1cc(Nc2cc(C)nc(C(C)(F)F)n2)c(OCc2ccnc(F)c2)cn1] | |
| | ROH: (2-fluoropyridin-4-yl)methanol | |
| 121 | N-(4-((2-(1,1-difluoroethyl)-6-methylpyrimidin-4-yl)amino)-5- | 419 |
| | ((3-methylisoxazol-5-yl)methoxy)pyridin-2-yl)acetamide | |
| | 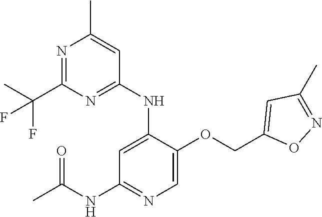 [SMILES: CC(=O)Nc1cc(Nc2cc(C)nc(C(C)(F)F)n2)c(OCc2cc(C)no2)cn1] | |
| | ROH: (3-methylisoxazol-5-yl)methanol | |
| 122 | N-(4-((2-(1,1-difluoroethyl)-6-methylpyrimidin-4-yl)amino)-5- | 405 |
| | (oxazol-5-ylmethoxy)pyridin-2-yl)acetamide | |
| | 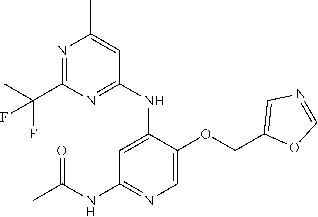 [SMILES: CC(=O)Nc1cc(Nc2cc(C)nc(C(C)(F)F)n2)c(OCc2cnco2)cn1] | |
| | ROH: oxazol-5-ylmethanol | |
| 123 | N-(4-((2-(1,1-difluoroethyl)-6-methylpyrimidin-4-yl)amino)-5- | 443 |
| | ((4,6-dimethylpyridin-2-yl)methoxy)pyridin-2-yl)acetamide | |
| | 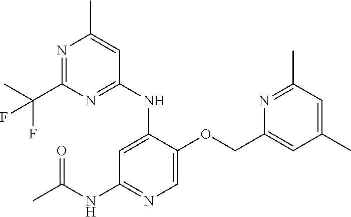 [SMILES: CC(=O)Nc1cc(Nc2cc(C)nc(C(C)(F)F)n2)c(OCc2cc(C)cc(C)n2)cn1] | |
| | ROH: (4,6-dimethylpyridin-2-yl)methanol | |
| 124 | (S)-N-(4-((2-(1,1-difluoroethyl)-6-methylpyrimidin-4-yl)amino)- | 438 |
| | 5-((2,2-dimethyl-1,3-dioxolan-4-yl)methoxy)pyridin-2- | |
| | yl)acetamide | |
| | 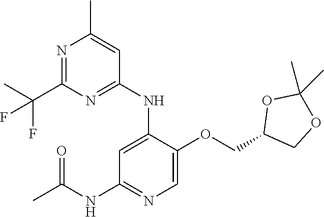 [SMILES: CC(=O)Nc1cc(Nc2cc(C)nc(C(C)(F)F)n2)c(OC[C@H]2COC(C)(C)O2)cn1] | |
| | ROH: (S)-(2,2-dimethyl-1,3-dioxolan-4-yl)methanol | |
| 125 | N-(4-((2-(1,1-difluoroethyl)-6-methylpyrimidin-4-yl)amino)-5- | 433 |
| | ((5-fluoropyridin-2-yl)methoxy)pyridin-2-yl)acetamide | |
| | 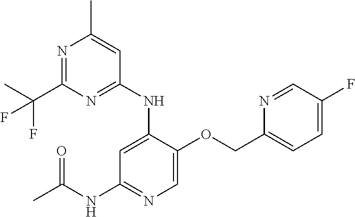 [SMILES: CC(=O)Nc1cc(Nc2cc(C)nc(C(C)(F)F)n2)c(OCc2ccc(F)cn2)cn1] | |
| | ROH: (5-fluoropyridin-2-yl)methanol | |
| 126 | N-(4-((2-(1,1-difluoroethyl)-6-methylpyrimidin-4-yl)amino)-5- | 416 |
| | (pyrazin-2-ylmethoxy)pyridin-2-yl)acetamide | |
| | 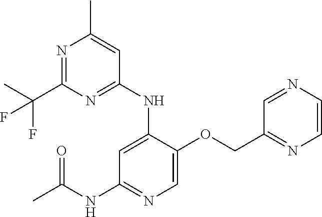 [SMILES: CC(=O)Nc1cc(Nc2cc(C)nc(C(C)(F)F)n2)c(OCc2cnccn2)cn1] | |
| | ROH: (pyrazin-2-ylmethanol | |
| 127 | N-(4-((2-(1,1-difluoroethyl)-6-methylpyrimidin-4-yl)amino)-5- | 445 |
| | ((5-methoxypyridin-2-yl)methoxy)pyridin-2-yl)acetamide | |
| | 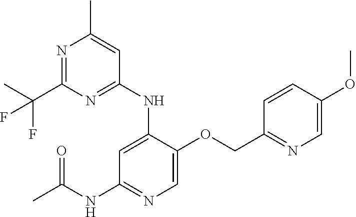 [SMILES: COc1ccc(COc2cnc(NC(C)=O)cc2Nc2cc(C)nc(C(C)(F)F)n2)nc1] | |
| | ROH: (5-methoxypyridin-2-yl)methanol | |
| 128 | N-(4-((2-(1,1-difluoroethyl)-6-methylpyrimidin-4-yl)amino)-5- | 445 |
| | ((4-methoxypyridin-2-yl)methoxy)pyridin-2-yl)acetamide | |
| | 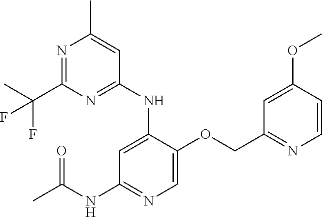 [SMILES: COc1ccnc(COc2cnc(NC(C)=O)cc2Nc2cc(C)nc(C(C)(F)F)n2)c1] | |
| | ROH: (4-methoxypyridin-2-yl)methanol | |
| 129 | N-(5-((4-cyanopyridin-2-yl)methoxy)-4-((2-(1,1-difluoroethyl)-6- | 440 |
| | methylpyrimidin-4-yl)amino)pyridin-2-yl)acetamide | |
| | 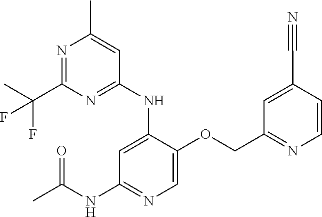 [SMILES: CC(=O)Nc1cc(Nc2cc(C)nc(C(C)(F)F)n2)c(OCc2cc(C#N)ccn2)cn1] | |
| | ROH: (4-cyanopyridin-2-yl)methanol | |
| 130 | N-(4-((2-(1,1-difluoroethyl)-6-methylpyrimidin-4-yl)amino)-5- | 429 |
| | ((5-methylpyridin-2-yl)methoxy)pyridin-2-yl)acetamide | |
| | 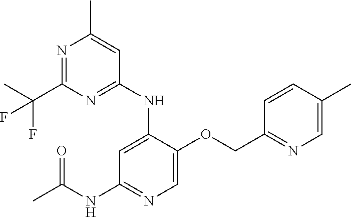 [SMILES: CC(=O)Nc1cc(Nc2cc(C)nc(C(C)(F)F)n2)c(OCc2ccc(C)cn2)cn1] | |
| | ROH: (5-methylpyridin-2-yl)methanol | |
| 131 | N-(5-((5-cyanopyridin-2-yl)methoxy)-4-((2-(1,1-difluoroethyl)-6- | 440 |
| | methylpyrimidin-4-yl)amino)pyridin-2-yl)acetamide | |
| | 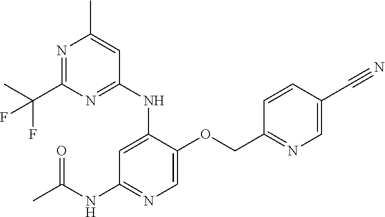 [SMILES: CC(=O)Nc1cc(Nc2cc(C)nc(C(C)(F)F)n2)c(OCc2ccc(C#N)cn2)cn1] | |
| | ROH: (5-cyanopyridin-2-yl)methanol | |
| 132 | N-(4-((2-(1,1-difluoroethyl)-6-methylpyrimidin-4-yl)amino)-5- | 445 |
| | ((6-methoxypyridin-2-y1)methoxy)pyridin-2-yl)acetamide | |
| | 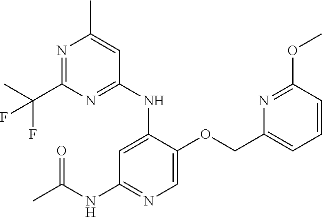 [SMILES: COc1cccc(COc2cnc(NC(C)=O)cc2Nc2cc(C)nc(C(C)(F)F)n2)n1] | |
| | ROH: (6-methoxypyridin-2-yl)methanol | |
| 133 | N-(4-((2-(1,1-difluoroethyl)-6-methylpyrimidin-4-yl)amino)-5- | 429 |
| | ((4-methylpyridin-2-yl)methoxy)pyridin-2-yl)acetamide | |
| | 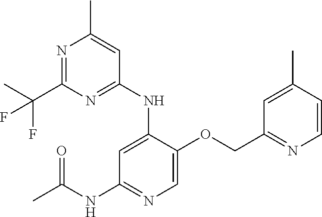 [SMILES: CC(=O)Nc1cc(Nc2cc(C)nc(C(C)(F)F)n2)c(OCc2cc(C)ccn2)cn1] | |
| | ROH: (4-methylpyridin-2-yl)methanol | |
| 134 | N-(4-((2-(1,1-difluoroethyl)-6-methylpyrimidin-4-yl)amino)-5- | 416 |
| | (pyrimidin-2-ylmethoxy)pyridin-2-yl)acetamide | |
| | 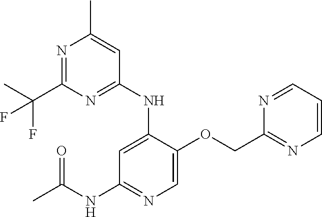 [SMILES: CC(=O)Nc1cc(Nc2cc(C)nc(C(C)(F)F)n2)c(OCc2ncccn2)cn1] | |
| | ROH: pyrimidin-2-ylmethanol | |
| 135 | N-(5-((1,4-dioxan-2-yl)methoxy)-4-((2-(1,1-difluoroethyl)-6- | 424 |
| | methylpyrimidin-4-yl)amino)pyridin-2-yl)acetamide | |
| | 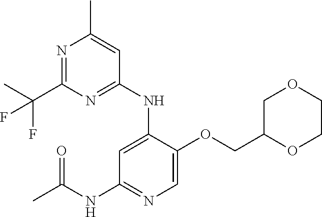 [SMILES: CC(=O)Nc1cc(Nc2cc(C)nc(C(C)(F)F)n2)c(OCC2COCCO2)cn1] | |
| | ROH: (1,4-dioxan-2-yl)methanol | |
| 136 | N-(4-((2-(1,1-difluoroethyl)-6-methylpyrimidin-4-yl)amino)-5- | 422 |
| | ((tetrahydro-2H-pyran-3-yl)methoxy)pyridin-2-yl)acetamide | |
| | 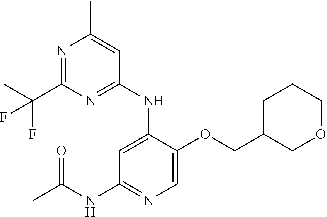 [SMILES: CC(=O)Nc1cc(Nc2cc(C)nc(C(C)(F)F)n2)c(OCC2CCCOC2)cn1] | |
| | ROH: (tetrahydro-2H-pyran-3-yl)methanol | |
| 137 | N-(4-((2-(1,1-difluoroethyl)-6-methylpyrimidin-4-yl)amino)-5-(3- | 410 |
| | methoxybutoxy)pyridin-2-yl)acetamide | |
| | 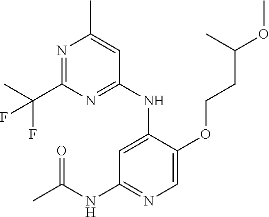 [SMILES: COC(C)CCOc1cnc(NC(C)=O)cc1Nc1cc(C)nc(C(C)(F)F)n1] | |
| | ROH: 3-methoxybutanol | |

### Example 138

### N-(4-((2-(1,1-difluoroethyl)-6-methylpyrimidin-4-yl)amino)-5-((1,5-dimethyl-1H-pyrazol-4-yl)methoxy)pyridin-2-yl)acetamide

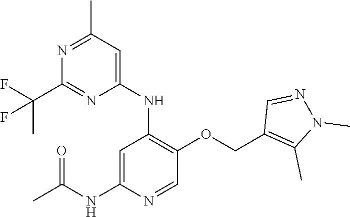 [SMILES: CC(=O)Nc1cc(Nc2cc(C)nc(C(C)(F)F)n2)c(OCc2cnn(C)c2C)cn1]

DIAD (28 mg, 0.142 mmol) was added to a solution of (1,5-dimethyl-1H-pyrazol-4-yl)methanol (23 mg, 0.190 mmol), PPh₃ (37 mg, 0.142 mmol) and N-(4-((2-(1,1-difluoroethyl)-6-methylpyrimidin-4-yl)amino)-5-hydroxypyridin-2-yl)acetamide (Preparation 279, 31 mg, 0.095 mmol) in THF (1 mL) and the reaction was stirred at rt overnight. The reaction was evaporated and purified by HPLC (Method U, Gradient 5-95%) to afford N-(4-((2-(1,1-difluoroethyl)-6-methylpyrimidin-4-yl)amino)-5-((1,5-dimethyl-1H-pyrazol-4-yl)methoxy)pyridin-2-yl)acetamide (2.9 mg, 7.3% yield) as tan solid. LCMS m/z=432 [M+1]⁺.

### Example 139

### N-(4-((6-cyclopropoxy-2-(1,1-difluoroethyl)pyrimidin-4-yl)amino)-5-((tetrahydrofuran-2-yl)methoxy)pyridin-2-yl)acetamide

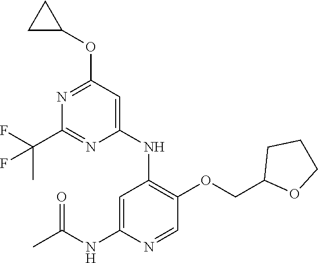 [SMILES: CC(=O)Nc1cc(Nc2cc(OC3CC3)nc(C(C)(F)F)n2)c(OCC2CCCO2)cn1]

DEAD (25 mg, 0.144 mmol) was added to a solution of (tetrahydrofuran-2-yl)methanol (10 mg, 0.096 mmol), THF (1 mL), PPh₃ (37 mg, 0.144 mmol), and N-(4-((6-cyclopropoxy-2-(1,1-difluoroethyl)pyrimidin-4-yl)amino)-5-hydroxypyridin-2-yl)acetamide (Preparation 281, 35 mg, 0.096 mmol) in THF (1 mL) and the reaction was stirred overnight. The reaction was evaporated and purified by HPLC (Method U, Gradient 5-95%) to afford N-(4-((6-cyclopropoxy-2-(1,1-difluoroethyl)pyrimidin-4-yl)amino)-5-((tetrahydrofuran-2-yl)methoxy)pyridin-2-yl)acetamide (5.7 mg, 13% yield) as a white solid. LCMS m/z=450 [M+H]⁺

### Example 140

### N-(4-((6-(1,1-difluoroethyl)pyridin-2-yl)amino)-5-(2-hydroxyethoxy)pyridin-2-yl)acetamide

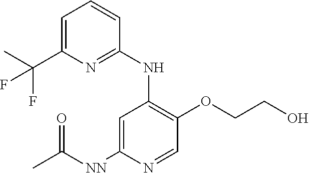

A solution of N-(4-((6-(1,1-difluoroethyl)pyridin-2-yl)amino)-5-(2-methoxyethoxy)pyridin-2-yl)acetamide (Preparation 56, 32 mg, 0.088 mmol) in DCM (1 mL) was cooled to 0° C. and tribromoborane (88 μL, 1 M in DCM) added slowly. The reaction was sonicated for 30 min to aid dissolution, additional tribromoborane (88 μL, 1 M in DCM) was added and the reaction stirred for 1 h at 0° C. The reaction was quenched by addition of saturated NaHCO₃, extracted with DCM (2×) and evaporated. The residue was purified by silica gel chromatography (0-20% MeOH in DCM) to give N-(4-((6-(1,1-difluoroethyl)pyridin-2-yl)amino)-5-(2-hydroxyethoxy)pyridin-2-yl)acetamide (14 mg, 45% yield). LCMS m/z=353.2 [M+H]⁺. ¹H NMR (400 MHz, CDCl₃+MeOH-d₄) δ ppm 8.92 (s, 1H), 7.47-7.59 (m, 2H), 7.24-7.30 (m, 1H), 6.99-7.07 (m, 1H), 6.89-6.97 (m, 1H), 3.92-3.99 (m, 2H), 3.73-3.84 (m, 2H), 1.82-2.01 (m, 6H).

### Example 141

### N-(4-((2-(2-oxabicyclo[2.1.1]hexan-4-yl)-6-methylpyrimidin-4-yl)amino)-5-(5-methyl-4,5,6,7-tetrahydropyrazolo[1,5-a]pyrazin-2-yl)pyridin-2-yl)acetamide

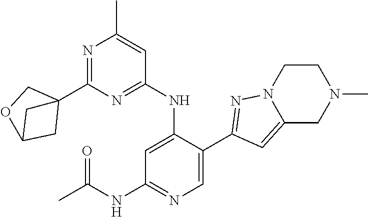 [SMILES: CC(=O)Nc1cc(Nc2cc(C)nc(C34COC(C3)C4)n2)c(-c2cc3n(n2)CCN(C)C3)cn1]

A vial containing 4-chloro-6-methyl-2-(1-methyl-2-oxabicyclo[2.1.1]hexan-4-yl)pyrimidine (Preparation 177, 69 mg, 0.326 mmol), N-(4-amino-5-(5-methyl-4,5,6,7-tetrahydropyrazolo[1,5-a]pyrazin-2-yl)pyridin-2-yl)acetamide trifluoroacetate (Preparation 474, 81 mg, 0.203 mmol), Pd₂dba₃ (20 mg, 22 mol), DIPEA (0.42 mL, 2.41 mmol) Cs₂CO₃ (211 mg, 0.669 mmol), Xantphos (29 mg, 51 mol) and Xantphos-Pd-G3 (40 mg, 43 mol) in dioxane (2 mL) was degassed then backfilled with N₂ then heated at 90° C. for 17 h. The mixture was cooled to rt then filtered through Celite®, washing through with EtOAc. The filtrate was concentrated in vacuo and the residue purified by silica gel chromatography (20-100% 3:1 EtOAc: EtOH in heptane). The product was suspended in EtOH, the heterogenous mixture was heated on the rotovap to 45° C. without vacuum for 20 mins, then filtered and the resulting solid dried to give N-(4-((2-(2-oxabicyclo[2.1.1]hexan-4-yl)-6-methylpyrimidin-4-yl)amino)-5-(5-methyl-4,5,6,7-tetrahydropyrazolo[1,5-a]pyrazin-2-yl)pyridin-2-yl)acetamide as a white solid. LCMS m/z=461.2 [M+H]⁺. ¹H NMR (500 MHz, DMSO-d₆) δ (ppm)=11.38 (s, 1H), 10.37 (s, 1H), 9.53 (s, 1H), 8.59 (s, 1H), 6.79 (s, 1H), 6.70 (s, 1H), 4.55 (s, 1H), 4.31-4.27 (m, 2H), 3.98 (s, 2H), 3.66 (s, 2H), 2.91 (t, J=5.5 Hz, 2H), 2.43 (s, 3H), 2.38-2.36 (m, 5H), 2.11 (s, 3H), 1.83 (dd, J=1.5, 4.3 Hz, 2H).

### Example 142

### N-(4′-((2-(2-oxabicyclo[2.1.1]hexan-1-yl)-6-methylpyrimidin-4-yl)amino)-5-((difluoromethoxy)methyl)-[2,3′-bipyridin]-6′-yl)acetamide

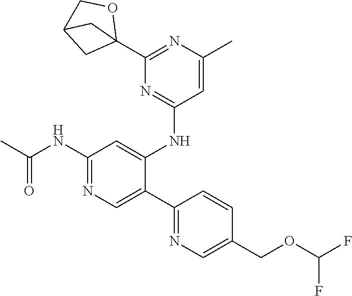 [SMILES: CC(=O)Nc1cc(Nc2cc(C)nc(C34CC(CO3)C4)n2)c(-c2ccc(COC(F)F)cn2)cn1]

A mixture of N-(4′-amino-5-((difluoromethoxy)methyl)-[2,3′-bipyridin]-6′-yl)acetamide hydrochloride (Preparation 477, 40 mg, 0.116 mmol), XantPhos-Pd-G3 (11 mg, 11.6 mol), Xantphos (6.7 mg, 11.6 mol), K₃PO₄ (86 mg, 0.406 mmol) in dioxane (2 mL) was degassed with N₂, 2-(2-oxabicyclo[2.1.1]hexan-1-yl)-4-chloro-6-methylpyrimidine (Preparation 176, 37 mg, 0.174 mmol) was then added and the reaction was heated at 100° C. for 3 h. The cooled reaction mixture was filtered and the filtrate was purified by chromatography on silica gel (10-90% EtOAc-EtOH 3:1 with 2% NH₄OH in heptane) to give N-(4′-((2-(2-oxabicyclo[2.1.1]hexan-1-yl)-6-methylpyrimidin-4-yl)amino)-5-((difluoromethoxy)methyl)-[2,3′-bipyridin]-6′-yl)acetamide (32 mg, 57% yield). LCMS m/z=483 [M+H]⁺ ¹H NMR (MeOH-d₄, 400 MHz) δ 9.43 (s, 1H), 8.77 (s, 1H), 8.68 (s, 1H), 7.99 (s, 2H), 6.8 (s, 1H), 6.57 (t, 1H, J=74.5 Hz), 5.05 (s, 2H), 4.63 (s, 1H), 4.10 (s, 2H), 2.4-2.5 (m, 5H), 2.21 (s, 3H), 1.96-1.98 (m, 2H).

### Example 143

### N-(5-cyclopropyl-4-((2-(1,1-difluoroethyl)-5-methoxypyrimidin-4-yl)amino)pyridin-2-yl)acetamide

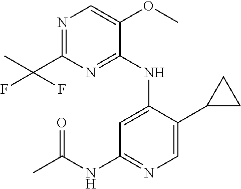 [SMILES: COc1cnc(C(C)(F)F)nc1Nc1cc(NC(C)=O)ncc1C1CC1]

N-(5-cyclopropyl-4-((2-(1,1-difluoroethyl)-5-methoxypyrimidin-4-yl)amino)pyridin-2-yl)acetamide was obtained as a white solid, 38 mg, 44%, from N-(4-amino-5-cyclopropylpyridin-2-yl)acetamide (Preparation 371) and 4-chloro-2-(1,1-difluoroethyl)-5-methoxypyrimidine (Preparation 154) following a similar procedure to that described in Example 141. LCMS m/z=364.1 [M+H]⁺. ¹H NMR (500 MHz, DMSO-d₆) δ (ppm)=10.22 (s, 1H), 8.81 (s, 1H), 8.68 (s, 1H), 8.57 (s, 1H), 7.92 (s, 1H), 3.94 (s, 3H), 2.09-2.00 (m, 6H), 1.96-1.90 (m, 1H), 0.96-0.91 (m, 2H), 0.62-0.58 (m, 2H).

### Example 144

### N-(4-((2-(1,1-difluoroethyl)pyrimidin-4-yl)amino)-5-(2,2-dimethyl-2,3-dihydro-[1,4]dioxino[2,3-b]pyridin-6-yl)pyridin-2-yl)acetamide trifluoroacetate

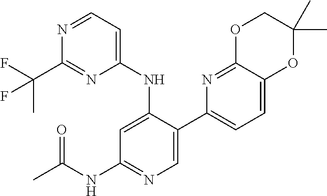 [SMILES: CC(=O)Nc1cc(Nc2ccnc(C(C)(F)F)n2)c(-c2ccc3c(n2)OCC(C)(C)O3)cn1]

A mixture of 2-chloro-5-(2,2-dimethyl-2,3-dihydrobenzo[b][1,4]dioxin-6-yl)pyridin-4-amine (Preparation 496, 50 mg, 0.16 mmol), 4-chloro-2-(1,1-difluoroethyl)pyrimidine (Preparation 148, 34 mg, 0.19 mmol), Cs₂CO₃ (104 mg, 0.32 mmol), Pd₂dba₃ (3.6 mg, 4.0 umol), and XantPhos (5.5 mg, 9.5 umol) in dioxane (2 mL) was stirred at 90° C. for 20 h. The reaction was removed from heating, cooled to rt, diluted with EtOAc, filtered through a 0.2 um syringe filter, and rinsed with EtOAc. The filtrate was concentrated to dryness. The crude mixture was purified via prep-HPLC (Method V, Gradient: 5-95%) to give N-(4-((2-(1,1-difluoroethyl)pyrimidin-4-yl)amino)-5-(2,2-dimethyl-2,3-dihydro-[1,4]dioxino[2,3-b]pyridin-6-yl)pyridin-2-yl)acetamide trifluoroacetate (32 mg, 45% yield). LCMS m/z=457.2 [M+H]+. 1H NMR (600 MHz, DMSO-d₆) δ (ppm)=11.87 (s, 1H), 10.54 (s, 1H), 9.10-9.06 (m, 1H), 8.65 (s, 1H), 8.56 (d, J=5.7 Hz, 1H), 7.55 (d, J=8.4 Hz, 1H), 7.42 (d, J=8.4 Hz, 1H), 6.95 (d, J=5.7 Hz, 1H), 4.23 (s, 2H), 2.12 (s, 3H), 2.11-2.02 (m, 3H), 1.36 (s, 6H).

### Example 145 to 149

The compounds in the following table were prepared from the appropriate amine and haloheterocycle, following a similar procedure to that described in Example 144.

| Example | |
|---|---|
| No | Name, Structure, Starting Materials (SM), Data |
| 145 | N-(5-cyano-4′-((2-(1,1-difluoroethyl)-6-methylpyrimidin-4-yl)amino)-[2,3′- |
| | bipyridin]-6′-yl)acetamide |
| | 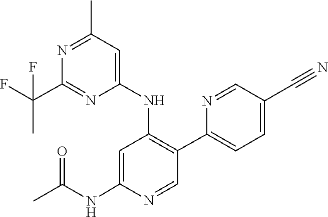 [SMILES: CC(=O)Nc1cc(Nc2cc(C)nc(C(C)(F)F)n2)c(-c2ccc(C#N)cn2)cn1] |
| | SM: N-(4′-amino-5-cyano-[2,3′-bipyridin]-6′-yl)acetamide hydrochloride |
| | (Preparation 451) and chloro-2-(1,1-difluoroethyl)-6-methylpyrimidine |
| | (Preparation 92) |
| | PE/EtOAc 1/0 to 0/1 column; (122.1 mg, 68.5% yield) as a yellow solid. |
| | LCMS m/z = 410.2 [M + H]⁺ ¹H NMR (400 MHz, CDCl₃) δ ppm: 12.29 (s, |
| | 1H), 9.46 (s, 1H), 9.00 (s, 1H), 8.61-8.69 (m, 2H), 8.14 (d, J = 6.8 Hz, 1H), |
| | 7.93 (d, J = 8.4 Hz, 1H), 6.82 (s, 1H), 2.57 (s, 3H), 2.29 (s, 3H), 2.16 (t, J = 18.8 |
| | Hz, 3H). |
| 146 | N-(5-cyano-4′-((2-(1,1-difluoroethyl)pyrimidin-4-yl)amino)-[2,3′-bipyridin]- |
| | 6′-yl)acetamide |
| | 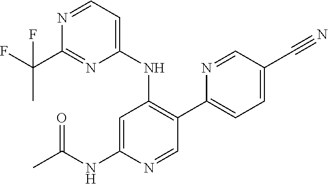 [SMILES: CC(=O)Nc1cc(Nc2ccnc(C(C)(F)F)n2)c(-c2ccc(C#N)cn2)cn1] |
| | SM: N-(4′-amino-5-cyano-[2,3′-bipyridin]-6′-yl)acetamide hydrochloride |
| | (Preparation 451) and 4-chloro-2-(1, 1-difluoroethyl)pyrimidine (Preparation |
| | 148) |
| | PE/EtOAc 1/0 to 0/1 silica gel column; White solid, 97.5 mg, 62.5% yield. |
| | LCMS m/z = 396.1 [M + H]⁺ 1H NMR (400 MHz, DMSO-d₆) 11.79 (s, 1H), |
| | 10.67 (s, 1H), 9.10-9.16 (m, 2H), 8.79 (s, 1H), 8.56 (d, J = 5.6 Hz, 1H), 8.41 |
| | (dd, J = 2.0, 8.8 Hz, 1H), 8.18 (d, J = 8.8 Hz, 1H), 7.18 (d, J = 6.0 Hz, 1H), |
| | 2.14 (s, 3H), 2.04 (t, J = 19.2 Hz, 3H). |
| 147 | N-(4-((6-(1, 1-difluoroethyl)pyridin-2-yl)amino)-5-(1-methyl-1H-pyrazol-3- |
| | yl)pyridin-2-yl)acetamide |
| | 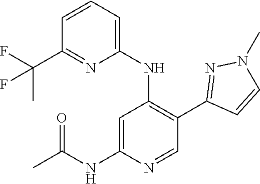 [SMILES: CC(=O)Nc1cc(Nc2cccc(C(C)(F)F)n2)c(-c2ccn(C)n2)cn1] |
| | SM: N-(4-amino-5-(1-methyl-1H-pyrazol-3-yl)pyridin-2-yl)acetamide |
| | (Preparation 481) and 2-bromo-6-(1,1-difluoroethyl)pyridine |
| | Prep-HPLC (Method B, 32-62%); 48 mg, 28.5% yield, as a white solid. LCMS |
| | m/z = 373.1 [M + H]⁺ ¹H NMR (400 MHz, DMSO-d₆) δ ppm 11.35 (s, 1H), |
| | 10.34 (s, 1H), 9.32 (s, 1H), 8.61 (s, 1H), 7.85-7.90 (m, 2H), 7.25 (d, J = 7.2 Hz, |
| | 1H), 7.18 (d, J = 8.4 Hz, 1H), 6.92 (d, J = 2.4 Hz, 1H), 4.01 (s, 3H), 2.16 (t, |
| | J = 19.6 Hz, 3H), 2.10 (s, 3H). |
| 148 | N-(4-((6-(1,1-difluoroethyl)-4-((1r,3r)-3-methoxycyclobutoxy)pyridin-2- |
| | yl)amino)-5-(1-methyl-1H-pyrazol-3-yl)pyridin-2-yl)acetamide |
| | 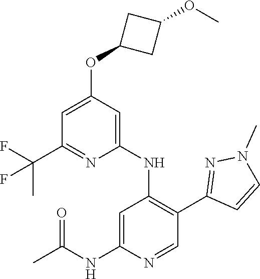 [SMILES: CO[C@H]1C[C@H](Oc2cc(Nc3cc(NC(C)=O)ncc3-c3ccn(C)n3)nc(C(C)(F)F)c2)C1] |
| | SM: N-(4-amino-5-(1-methyl-1H-pyrazol-3-yl)pyridin-2-yl)acetamide |
| | (Preparation 481) and 2-chloro-6-(1,1-difluoroethyl)-4-((1r,3r)-3- |
| | methoxycyclobutoxy)pyridine (Preparation 88) |
| | prep-HPLC (Method R, Gradient 39-59%) 80.0 mg, 30.2 % yield as a white |
| | solid. LCMS m/z = 473.3 [M + H]⁺. 1H NMR (400 MHz, CDCl₃) δ ppm: 10.96 |
| | (s, 1H), 9.20 (s, 1H), 8.41 (s, 1H), 8.05 (br s, 1H), 7.44 (d, J-2.4 Hz, 1H), |
| | 6.75 (d, J = 2.0 Hz, 1H), 6.61 (d, J-2.4 Hz, 1H), 6.35 (d, J = 1.6 Hz, 1H), 4.93- |
| | 4.97 (m, 1H), 4.14-4.18 (m, 1H), 4.01 (s, 3H), 3.28 (s, 3H), 2.44-2.52 (m, |
| | 4H), 2.10-2.20 (m, 6H). |
| 149 | N-(4-((6-(1,2-difluoroethyl)pyridin-2-yl)amino)-5-(1-methyl-1H-pyrazol-3- |
| | yl)pyridin-2-yl)acetamide |
| | 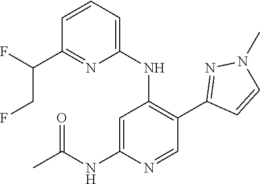 [SMILES: CC(=O)Nc1cc(Nc2cccc(C(F)CF)n2)c(-c2ccn(C)n2)cn1] |
| | SM: N-(4-amino-5-(1-methyl-1H-pyrazol-3-yl)pyridin-2-yl)acetamide |
| | (Preparation 481) and 2-bromo-6-(1,2-difluoroethyl)pyridine (Preparation |
| | 185) |
| | Prep HPLC (Method R, Gradient 26-56%) 65.0 mg, 40.4% yield as a yellow |
| | solid. LCMS m/z = 373.0 [M + H]⁺ 1H NMR (400 MHz, CDCl₃) δ ppm 11.11 |
| | (s, 1H), 9.52 (s, 1H), 8.42 (s, 1H), 8.10 (br s, 1H), 7.65-7.69 (m, 1H), 7.44 (d, |
| | J = 2.4 Hz, 1H), 7.13 (d, J = 7.6 Hz, 1H), 6.87 (d, J = 8.0 Hz, 1H), 6.63 (d, J = 2.4 |
| | Hz, 1H), 5.86-5.92 (m, 1H), 5.01-5.24 (m, 2H), 4.02 (s, 3H), 2.22 (s, 3H). |

### Example 150

### N-(4′-((2-(1,1-difluoroethyl)-6-methylpyrimidin-4-yl)amino)-6-(difluoromethoxy)-[2,3′-bipyridin]-6′-yl)acetamide

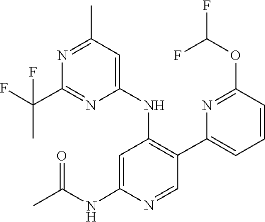 [SMILES: CC(=O)Nc1cc(Nc2cc(C)nc(C(C)(F)F)n2)c(-c2cccc(OC(F)F)n2)cn1]

To a solution of N-(4′-amino-6-(difluoromethoxy)-[2,3′-bipyridin]-6′-yl)acetamide hydrochloride (Preparation 452, 140.0 mg, 0.476 mmol) and 4-chloro-2-(1,1-difluoroethyl)-6-methylpyrimidine (Preparation 92, 100.8 mg, 0.523 mmol) in dioxane (10 mL) was added K₃PO₄ (606.0 mg, 2.85 mmol), Xantphos (27.5 mg, 47.6 umol) and Pd₂(dba)₃ (43.6 mg, 47.6 umol). The reaction was stirred at 100° C. for 6 h under N₂. The cooled mixture was filtered and concentrated under reduced pressure to give a residue, which was purified by prep-HPLC (Method AA, Gradient 32-62%) to give N-(4′-((2-(1,1-difluoroethyl)-6-methylpyrimidin-4-yl)amino)-6-(difluoromethoxy)-[2,3′-bipyridin]-6′-yl)acetamide (26.3 mg, 12.3% yield) as a white solid. LCMS m/z=451.1 [M+H]⁺ ¹H NMR (400 MHz, DMSO-d₆) δ ppm 10.61 (s, 1H), 10.21 (s, 1H), 8.82 (s, 1H), 8.58 (s, 1H), 7.96-8.03 (m, 1H), 7.56-7.95 (m, 2H), 7.06 (d, J=8.0 Hz, 1H), 6.82 (s, 1H), 2.37 (s, 3H), 2.12 (s, 3H), 1.96 (t, J=18.8 Hz, 3H).

### Examples 151 to 157

The compounds in the following table were prepared from the appropriate aromatic amine and 4-chloro-2-(1,1-difluoroethyl)-6-methylpyrimidine (Preparation 92), following a similar procedure to that described in Example 150.

| Example | |
|---|---|
| No | Name, Structure, Starting Materials (SM), Data |
| 151 | N-(4-((2-(1,1-difluoroethyl)-6-methylpyrimidin-4-yl)amino)-5-(3- |
| | (dimethylamino)propyl)pyridin-2-yl)acetamide |
| | 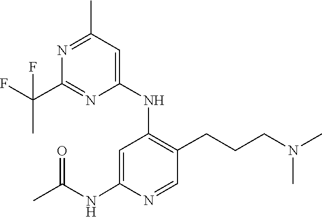 [SMILES: CC(=O)Nc1cc(Nc2cc(C)nc(C(C)(F)F)n2)c(CCCN(C)C)cn1] |
| | SM: N-(4-amino-5-(3-(dimethylamino)propyl)pyridin-2-yl)acetamide |
| | (Preparation 369) |
| | Prep-HPLC (Method B, 30-59%). 52.1 mg, 31.4% yield as a white solid. |
| | LCMS m/z = 393.2 [M + H]⁺. ¹H NMR (400 MHz, CDCl₃) δ ppm: 11.62 (s, |
| | 1H), 8.51 (s, 1H), 8.10 (s, 1H), 8.04 (s, 1H), 6.79 (s, 1H), 2.69 (t, J = 6.0 Hz, |
| | 2H), 2.49 (s, 3H), 2.33 (s, 6H), 2.20-2.22 (m, 5H), 2.04 (t, J = 18.8 Hz, 3H), |
| | 1.87-1.90 (m, 2H). |
| 152 | N-(4-((2-(1, 1-difluoroethyl)-6-methylpyrimidin-4-yl)amino)-5-(3- |
| | methoxypropyl)pyridin-2-yl)acetamide |
| | 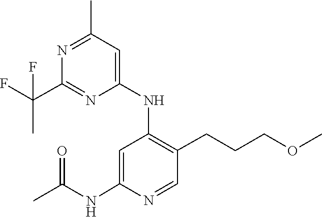 [SMILES: COCCCc1cnc(NC(C)=O)cc1Nc1cc(C)nc(C(C)(F)F)n1] |
| | SM: N-(4-amino-5-(3-methoxypropyl)pyridin-2-yl)acetamide hydrochloride |
| | (Preparation 370) |
| | Prep-HPLC (Method B, 30-60%). 28.2 mg, 19.3% yield as a white solid. LCMS |
| | m/z = 380.1 [M + H]⁺. ¹H NMR (400 MHz, CDCl₃) δ ppm: 9.67 (brs, 1H), 8.40- |
| | 8.44 (m, 2H), 8.05 (brs, 1H), 6.89 (s, 1H), 3.49 (s, 3H), 3.37 (t, J = 5.2 Hz, 2H), |
| | 2.73-2.74 (m, 2H), 2.52 (s, 3H), 2.21 (s, 3H), 2.07 (t, J = 18.8 Hz, 3H), 1.88- |
| | 1.93 (m, 2H). |
| 153 | N-(5-(cyclopropyldifluoromethyl)-4-((2-(1,1-difluoroethyl)-6- |
| | methylpyrimidin-4-yl)amino)pyridin-2-yl)acetamide |
| | 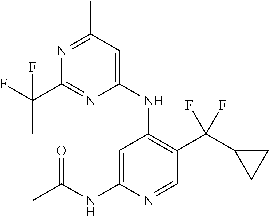 [SMILES: CC(=O)Nc1cc(Nc2cc(C)nc(C(C)(F)F)n2)c(C(F)(F)C2CC2)cn1] |
| | SM: N-(4-amino-5-(cyclopropyldifluoromethyl)pyridin-2-yl)acetamide |
| | (Preparation 381) |
| | prep-HPLC (Method R, Gradient 27-57%) 5.7 mg, 11.5% yield as a white |
| | solid. LCMS m/z = 398.1 [M + H]⁺ ¹H NMR (400 MHz, DMSO-d₆) δ ppm: |
| | 10.69 (s, 1H), 8.68-8.74 (m, 2H), 8.48 (s, 1H), 7.11 (s, 1H), 2.40 (s, 3H), 2.10 |
| | (s, 3H), 1.87-2.00 (m, 4H), 0.64-0.73 (m, 4H). |
| 154 | N-(4-((2-(1,1-difluoroethyl)-6-methylpyrimidin-4-yl)amino)-5-(1,1- |
| | difluoropropyl)pyridin-2-yl)acetamide |
| | 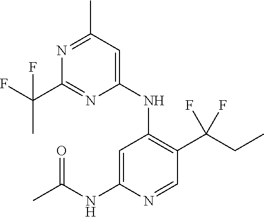 [SMILES: CCC(F)(F)c1cnc(NC(C)=O)cc1Nc1cc(C)nc(C(C)(F)F)n1] |
| | SM: N-(4-amino-5-(1,1-difluoropropyl)pyridin-2-yl)acetamide |
| | trifluoroacetate (Preparation 382) |
| | prep-HPLC (Method R, Gradient 34-64%) 16.1 mg, 43.5% yield as a white |
| | solid. LCMS m/z = 386.2 [M + H]⁺ ¹H NMR (400 MHz, CDCl₃) δ ppm: 8.84 (s, |
| | 1H), 8.27 (s, 1H), 8.19 (s, 1H), 7.54 (s, 1H), 6.91 (s, 1H), 2.55 (s, 3H), 2.23- |
| | 2.29 (m, 5H), 2.09 (t, J = 18.8 Hz, 3H), 1.05 (t, J = 7.6 Hz, 3H). |
| 155 | N-(4-((2-(1,1-difluoroethyl)-6-methylpyrimidin-4-yl)amino)-5-(6-methoxy-2- |
| | methylpyrimidin-4-yl)pyridin-2-yl)acetamide |
| | 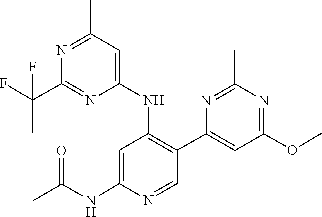 [SMILES: COc1cc(-c2cnc(NC(C)=O)cc2Nc2cc(C)nc(C(C)(F)F)n2)nc(C)n1] |
| | SM: N-(4-amino-5-(6-methoxy-2-methylpyrimidin-4-yl)pyridin-2- |
| | yl)acetamide hydrochloride (Preparation 462) |
| | prep-HPLC (Method S, Gradient 38-66%) 20.5 mg, 16.4% yield as a white |
| | solid. LCMS m/z = 430.1 [M + H]⁺ 1H NMR (500 MHz, CDCl₃) δ ppm: 13.01 |
| | (s, 1H), 9.33 (s, 1H), 8.56 (s, 1H), 8.04 (s, 1H), 6.93 (s, 1H), 6.71 (s, 1H), 4.04 |
| | (s, 3H), 2.77 (s, 3H), 2.56 (s, 3H), 2.25 (s, 3H), 2.17 (t, J = 18.5 Hz, 3H). |
| 156 | N-(4-((2-(1,1-difluoroethyl)-6-methylpyrimidin-4-yl)amino)-5-(2-methoxy-6- |
| | methylpyrimidin-4-yl)pyridin-2-yl)acetamide |
| | 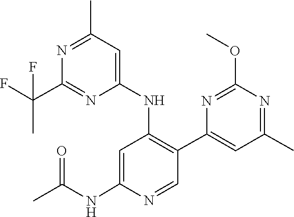 [SMILES: COc1nc(C)cc(-c2cnc(NC(C)=O)cc2Nc2cc(C)nc(C(C)(F)F)n2)n1] |
| | SM: N-(4-amino-5-(2-methoxy-6-methylpyrimidin-4-yl)pyridin-2- |
| | yl)acetamide hydrochloride (Preparation 463) |
| | Prep-HPLC (Method G, 32-62%). 46.81 mg, 31.2% yield as a white solid. |
| | LCMS m/z = 430.2 [M + H]⁺. ¹H NMR (500 MHz, DMSO-d₆) δ ppm: 11.74 (s, |
| | 1H), 10.67 (s, 1H), 9.06 (s, 1H), 8.79 (s, 1H), 7.63 (s, 1H), 6.91 (s, 1H), 4.00 |
| | (s, 3H), 2.46 (s, 3H), 2.43 (s, 3H), 2.13 (s, 3H), 2.04 (t, J = 19.0 Hz, 3H) |
| 157 | N-(4-((2-(1,1-difluoroethyl)-6-methylpyrimidin-4-yl)amino)-5-(1-methyl-1H- |
| | 1,2,4-triazol-3-yl)pyridin-2-yl)acetamide |
| | 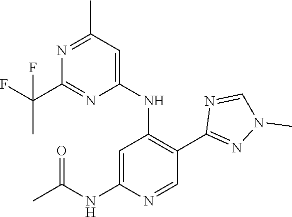 [SMILES: CC(=O)Nc1cc(Nc2cc(C)nc(C(C)(F)F)n2)c(-c2ncn(C)n2)cn1] |
| | SM: N-(4-amino-5-(1-methyl-1H-1,2,4-triazol-3-yl)pyridin-2-yl)acetamide |
| | hydrochloride (Preparation 466) |
| | Prep-HPLC (Method G, Gradient 30-60%). LCMS m/z = 389.1 [M + H]⁺ |
| | 1H NMR: (400 MHz, DMSO-d₆) δ ppm: 11.35 (s, 1H), 10.56 (s, 1H), 9.35 (s, |
| | 1H), 8.92 (s, 1H), 8.76 (s, 1H), 7.07 (s, 1H), 4.02 (s, 3H), 2.46 (s, 3H), 2.08- |
| | 2.18 (m, 6H). |

The compounds in the following table were prepared from the appropriate aromatic amine and haloheterocycle, following a similar procedure to that described in Example 150.

| Example | |
|---|---|
| No | Name, Structure, Starting Materials (SM), Data |
| 158 | N-(4-((6-(1, 1-difluoroethyl)pyridin-2-yl)amino)-5-(1-methyl-6-oxo-1,6- |
| | dihydropyridazin-3-yl)pyridin-2-yl)acetamide |
| | 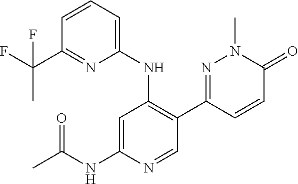 [SMILES: CC(=O)Nc1cc(Nc2cccc(C(C)(F)F)n2)c(-c2ccc(=O)n(C)n2)cn1] |
| | SM: N-(4-amino-5-(1-methyl-6-oxo-1,6-dihydropyridazin-3-yl)pyridin-2- |
| | yl)acetamide (Preparation 475) and 2-bromo-6-(1,1-difluoroethyl)pyridine |
| | Prep-HPLC (Method B, 27-57%). 66.0 mg, 57.0 % yield as a white solid. |
| | LCMS m/z = 401.1 [M + H]⁺ 1H NMR (400 MHz, DMSO-d₆) δ ppm 10.49 (s, |
| | 1H), 9.95 (s, 1H), 9.01 (s, 1H), 8.43 (s, 1H), 7.87-7.82 (m, 2H), 7.22 (d, J = |
| | 7.6 Hz, 1H), 7.15 (d, J = 8.4 Hz, 1H), 7.00 (d, J = 9.6 Hz, 1H), 3.76 (s, 3H), |
| | 2.10 (s, 3H), 2.03 (t, J = 19.6 Hz, 3H) |
| 159 | N-(4-((6-(1,1-difluoroethyl)pyridin-2-yl)amino)-5-(4-methyl-5-oxo-4,5- |
| | dihydropyrazin-2-yl)pyridin-2-yl)acetamide |
| | 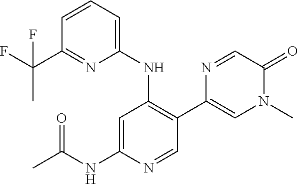 [SMILES: CC(=O)Nc1cc(Nc2cccc(C(C)(F)F)n2)c(-c2cn(C)c(=O)cn2)cn1] |
| | SM: N-(4-amino-5-(4-methyl-5-oxo-4,5-dihydropyrazin-2-yl)pyridin-2- |
| | yl)acetamide hydrochloride (Preparation 455) and 2-bromo-6-(1,1- |
| | difluoroethyl)pyridine |
| | Prep-HPLC (Method B, 23-51%). 35 mg, 12.6% as a white solid. LCMS m/z = |
| | 401.2 [M + H]⁺. ¹H NMR (400 MHz, DMSO-d₆) δ ppm: 10.36 (s, 1H), 9.67 |
| | (s, 1H), 9.05 (s, 1H), 8.26 (s, 1H), 8.16 (s, 1H), 8.13 (s, 1H), 7.81 (t, J = 8.0 Hz, |
| | 1H), 7.19 (d, J = 7.5 Hz, 1H), 7.12 (d, J = 8.5 Hz, 1H), 3.52 (s, 3H), 2.04-2.12 (m, |
| | 6H). |
| 160 | N-(4-((6-(1,1-difluoroethyl)pyridin-2-yl)amino)-5-(1-methyl-2-oxo-1,2- |
| | dihydropyrimidin-4-yl)pyridin-2-yl)acetamide |
| | 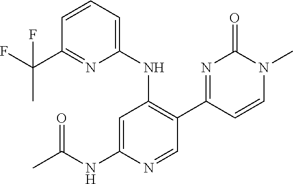 [SMILES: CC(=O)Nc1cc(Nc2cccc(C(C)(F)F)n2)c(-c2ccn(C)c(=O)n2)cn1] |
| | SM: N-(4-amino-5-(1-methyl-2-oxo-1,2-dihydropyrimidin-4-yl)pyridin-2- |
| | yl)acetamide trifluoroacetate (Preparation 476) and 2-bromo-6-(1,1- |
| | difluoroethyl)pyridine |
| | Prep-HPLC (Method B, 22-51%). 35 mg, 32.5% yield as a white solid. LCMS |
| | m/z = 401.1 [M + H]⁺. ¹H NMR (500 MHz, DMSO-d₆) δ ppm: 13.66 (s, 1H), |
| | 10.56 (s, 1H), 9.33 (s, 1H), 8.92 (s, 1H), 8.28 (d, J = 7.0 Hz, 1H), 7.94 (t, J = 8.0 |
| | Hz, 1H), 7.30 (d, J = 7.5 Hz, 1H), 7.22-7.26 (m, 1H), 7.02 (d, J = 8.0 Hz, 1H), |
| | 3.49 (s, 3H), 2.11-2.21 (m, 6H). |
| 161 | N-(4-((6-(1,1-difluoroethyl)pyridin-2-yl)amino)-5-(1-methyl-6-oxo-1,6- |
| | dihydropyrimidin-4-yl)pyridin-2-yl)acetamide |
| | 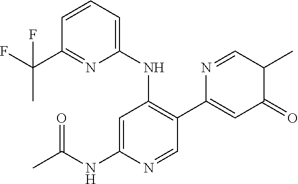 [SMILES: CC(=O)Nc1cc(Nc2cccc(C(C)(F)F)n2)c(C2=CC(=O)C(C)C=N2)cn1] |
| | SM: N-(4-amino-5-(1-methyl-6-oxo-1,6-dihydropyrimidin-4-yl)pyridin-2- |
| | yl)acetamide hydrochloride (Preparation 456) and 2-bromo-6-(1,1- |
| | difluoroethyl)pyridine |
| | Prep-HPLC (Method B, 25-55%). 10.7 mg, 29.2% yield as white solid. LCMS |
| | m/z = 401.1 [M + H]⁺. ¹H NMR (400 MHz, DMSO-d₆) δ ppm: 11.07 (s, 1H), |
| | 10.50 (s, 1H), 9.12 (s, 1H), 8.70 (s, 1H), 8.53 (s, 1H), 7.87 (t, J = 7.6 Hz, 1H), |
| | 7.25 (d, J = 7.6 Hz, 1H), 7.16 (d, J = 8.4 Hz, 1H), 6.87 (s, 1H), 3.47 (s, 3H), 2.05- |
| | 2.15 (m, 6H). |
| 162 | N-(4-((6-(1,1-difluoroethyl)pyridin-2-yl)amino)-5-(6-methoxypyrimidin-4- |
| | yl)pyridin-2-yl)acetamide |
| | 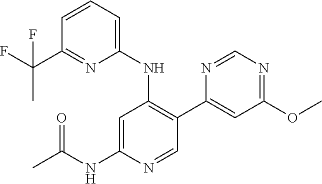 [SMILES: COc1cc(-c2cnc(NC(C)=O)cc2Nc2cccc(C(C)(F)F)n2)ncn1] |
| | SM: N-(4-amino-5-(6-methoxypyrimidin-4-yl)pyridin-2-yl)acetamide |
| | hydrochloride (Preparation 458) and 2-bromo-6-(1,1-difluoroethyl)pyridine |
| | Prep-HPLC (Method C, 40% - 70%) . 25 mg, 40.5% yield as a yellow solid. |
| | LCMS m/z= 401.1 [M + H]⁺. ¹H NMR (500 MHz, CDCl₃) δ ppm 12.39 (s, 1H), |
| | 9.34 (s, 1H), 8.87 (s, 1H), 8.49 (s, 2H), 7.73-7.76 (m, 1H), 7.31 (d, J = 7.5 Hz, |
| | 1H), 7.10 (s, 1H), 7.05 (d, J-8.0 Hz, 1H), 4.06 (s, 3H), 2.25 (s, 3H), 2.17 (t, |
| | J = 19.0 Hz, 3H). |
| 163 | N-(4-((2-(1, 1-difluoroethyl)pyrimidin-4-yl)amino)-5-(6-methoxy-2- |
| | methylpyrimidin-4-yl)pyridin-2-yl)acetamide |
| | 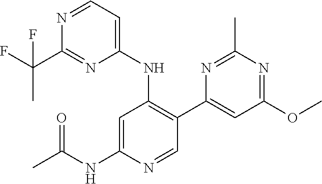 [SMILES: COc1cc(-c2cnc(NC(C)=O)cc2Nc2ccnc(C(C)(F)F)n2)nc(C)n1] |
| | SM: N-(4-amino-5-(6-methoxy-2-methylpyrimidin-4-yl)pyridin-2- |
| | yl)acetamide hydrochloride (Preparation 462) and 4-chloro-2-(1,1- |
| | difluoroethyl)pyrimidine (Preparation 148) |
| | prep-HPLC (Method S, Gradient 34-63%) 13.2 mg, 10.9% yield as a white |
| | solid. LCMS m/z = 416.2 [M + H]⁺ ¹H NMR (500 MHz, CDCl₃) δ ppm: 13.28 |
| | (s, 1H), 9.40 (s, 1H), 8.57-8.59 (m, 2H), 8.20 (br s, 1H), 6.96 (s, 1H), 6.89 (d, |
| | J = 5.5 Hz, 1H), 4.05 (s, 3H), 2.77 (s, 3H), 2.27 (s, 3H), 2.19 (t, J = 18.5 Hz, 3H). |
| 164 | N-(4-((2-(1, 1-difluoroethyl)pyrimidin-4-yl)amino)-5-(2-methoxy-6- |
| | methylpyrimidin-4-yl)pyridin-2-yl)acetamide |
| | 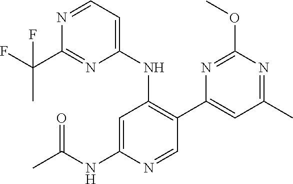 [SMILES: COc1nc(C)cc(-c2cnc(NC(C)=O)cc2Nc2ccnc(C(C)(F)F)n2)n1] |
| | SM: N-(4-amino-5-(2-methoxypyrimidin-4-yl)pyridin-2-yl)acetamide |
| | hydrochloride (Preparation 457) and 4-chloro-2-(1,1-difluoroethyl)pyrimidine |
| | (Preparation 148) |
| | Prep-HPLC (Method G, 30-60%). 40.6 mg, 28.3% yield as a white solid. |
| | LCMS m/z = 416.2 [M + H]⁺ ¹H NMR (400 MHz, DMSO-d₆) δ ppm: 11.77 (s, |
| | 1H), 10.70 (s, 1H), 9.08 (s, 1H), 8.80 (s, 1H), 8.59 (d, J = 6.4 Hz, 1H), 7.63 (s, |
| | 1H), 7.06 (d, J-5.6 Hz, 1H), 3.99 (s, 3H), 2.46 (s, 3H), 2.06 (s, 3H), 1.95-2.05 |
| | (m, 3H). |
| 165 | N-(4-((2-(1,1-difluoroethyl)pyrimidin-4-yl)amino)-5-(1-methyl-1H-1,2,4- |
| | triazol-3-yl)pyridin-2-yl)acetamide |
| | 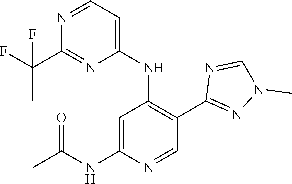 [SMILES: CC(=O)Nc1cc(Nc2ccnc(C(C)(F)F)n2)c(-c2ncn(C)n2)cn1] |
| | SM: N-(4-amino-5-(1-methyl-1H-1,2,4-triazol-3-yl)pyridin-2-yl)acetamide |
| | hydrochloride (Preparation 466) and 4-chloro-2-(1,1-difluoroethyl)pyrimidine |
| | (Preparation 148) |
| | Prep-HPLC (Method C, 17% to 47%) . 10 mg, 8.9% yield as a yellow solid. |
| | LCMS m/z = 375.1 [M + H]⁺. 1HNMR (400 MHz, CDCl₃) δ: ppm 11.41 (s, 1H), |
| | 9.39 (s, 1H), 9.05 (s, 1H), 8.56 (d, J = 6.0 Hz, 1H), 8.16 (s, 1H), 8.03 (s, 1H), |
| | 6.98 (d, J = 5.6 Hz, 1H), 4.05 (S, 3H) , 2.15-2.25 (m, 6H). |
| 166 | N-(4-((2-(1,1-difluoroethyl)-6-ethylpyrimidin-4-yl)amino)-5-(1-methyl-1H- |
| | 1,2,4-triazol-3-yl)pyridin-2-yl)acetamide |
| | 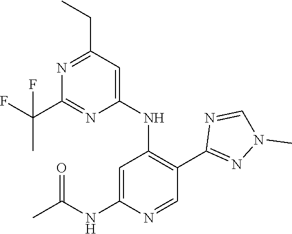 [SMILES: CCc1cc(Nc2cc(NC(C)=O)ncc2-c2ncn(C)n2)nc(C(C)(F)F)n1] |
| | SM: N-(4-amino-5-(1-methyl-1H-1,2,4-triazol-3-yl)pyridin-2-yl)acetamide |
| | hydrochloride (Preparation 466) and 4-chloro-2-(1, 1-difluoroethyl)-6- |
| | ethylpyrimidine (Preparation 94) |
| | Prep-HPLC (Method R, Gradient 33-53%) 28 mg, 18.70% yield as a white |
| | solid. LCMS m/z = 403.2 [M + H]⁺ 1H NMR (400 MHz, CDCl₃) δ ppm 11.27 |
| | (s, 1H), 9.36 (s, 1H), 9.02 (s, 1H), 8.16 (s, 1H) 8.13-8.19 (m, 2H), 6.83 (s, 1H), |
| | 4.05 (s, 3H), 2.82 (q, J = 7.2 Hz, 2H), 2.14-2.24 (m, 6H), 1.35 (t, J = 7.6 Hz, 3H) |
| 167 | N-(4-((2-(1,1-difluoroethyl)-6-ethylpyrimidin-4-yl)amino)-5-(5-methyl-1,3,4- |
| | thiadiazol-2-yl)pyridin-2-yl)acetamide |
| | 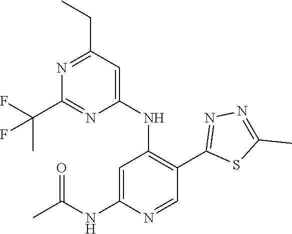 [SMILES: CCc1cc(Nc2cc(NC(C)=O)ncc2-c2nnc(C)s2)nc(C(C)(F)F)n1] |
| | SM: N-(4-amino-5-(5-methyl-1,3,4-thiadiazol-2-yl)pyridin-2-yl)acetamide |
| | hydrochloride (Preparation 467) and 4-chloro-2-(1,1-difluoroethyl)-6- |
| | ethylpyrimidine (Preparation 94) |
| | Prep-HPLC (Method B, 32-60%). 25.9 mg, 30.8% yield as a white solid. LCMS |
| | m/z = 420.1 [M + H]⁺ 1H NMR: (500 MHz, CDCl₃) δ ppm: 9.71 (s, 1H), 8.45 |
| | (s, 1H), 8.40 (br s, 1H), 6.86 (s, 1H), 2.81-2.87 (m, 5H), 2.27 (s, 3H), 2.20 (t, |
| | J = 19.0 Hz, 3H), 1.34 (t, J = 7.5 Hz, 3H). |
| 168 | N-(4-((2-(1,1-difluoroethyl)-6-ethylpyrimidin-4-yl)amino)-5-(5-methyl-1,3,4- |
| | oxadiazol-2-yl)pyridin-2-yl)acetamide |
| | 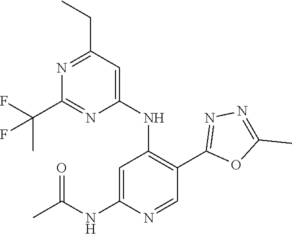 [SMILES: CCc1cc(Nc2cc(NC(C)=O)ncc2-c2nnc(C)o2)nc(C(C)(F)F)n1] |
| | SM: N-(4-amino-5-(5-methyl-1,3,4-oxadiazol-2-yl)pyridin-2-yl)acetamide |
| | hydrochloride (Preparation 468) and 4-chloro-2-(1, 1-difluoroethyl)-6- |
| | ethylpyrimidine (Preparation 94) |
| | Prep-HPLC (Method C, Gradient 29-59%) . 35 mg, 40.5% yield as a yellow |
| | solid. LCMS m/z = 404.1 [M + H]⁺ 1H NMR (500 MHz, CDCl₃) δ: ppm 10.98 |
| | (s, 1H), 9.70 (s, 1H), 8.71 (s, 1H), 8.20 (s, 1H), 6.86 (s, 1H), 2.85 (q, J = 7.5 Hz, |
| | 2H), 2.68 (s, 3H), 2.24-2.27 (m, 3H), 2.17-2.21 (m, 3H), 1.35 (t, J = 7.5 Hz, 3H). |
| 169 | N-(4-((2-(1, 1-difluoroethyl)-6-ethylpyrimidin-4-yl)amino)-5-(3-methyl-1,2,4- |
| | thiadiazol-5-yl)pyridin-2-yl)acetamide |
| | 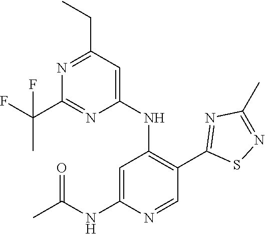 [SMILES: CCc1cc(Nc2cc(NC(C)=O)ncc2-c2nc(C)ns2)nc(C(C)(F)F)n1] |
| | SM: N-(4-amino-5-(3-methyl-1,2,4-thiadiazol-5-yl)pyridin-2-yl)acetamide |
| | hydrochloride (Preparation 469) and 4-chloro-2-(1,1-difluoroethyl)-6- |
| | ethylpyrimidine (Preparation 94) |
| | Prep-HPLC (Method B, 40-67%). 8.3 mg, 9.4% yield as a white solid. |
| | LCMS m/z = 420.1 [M + H]⁺ 1H NMR (500 MHz, CDCl₃) δ ppm: 11.95 (s, |
| | 1H), 9.59 (s, 1H), 8.60 (s, 1H), 8.29 (s, 1H), 6.77 (s, 1H), 2.84-2.88 (m, 2H), |
| | 2.81 (s, 3H), 2.27 (s, 3H), 2.20 (t, J = 18.5 Hz, 3H), 1.36 (t, J = 7.5 Hz, 3H). |
| 170 | N-(4-((2-(1, 1-difluoroethyl)-6-ethylpyrimidin-4-yl)amino)-5-(5-methyl-1,2,4- |
| | oxadiazol-3-yl)pyridin-2-yl)acetamide |
| | 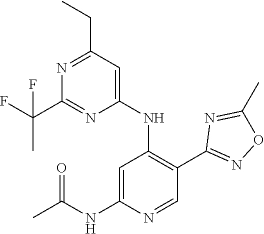 [SMILES: CCc1cc(Nc2cc(NC(C)=O)ncc2-c2noc(C)n2)nc(C(C)(F)F)n1] |
| | SM: N-(4-amino-5-(5-methyl-1,2,4-oxadiazol-3-yl)pyridin-2-yl)acetamide |
| | hydrochloride (Preparation 470) and 4-chloro-2-(1, 1-difluoroethyl)-6- |
| | ethylpyrimidine (Preparation 94) |
| | Prep-HPLC (Method C, Gradient 29-59%) . 67 mg, 38.7% yield as a yellow |
| | solid. LCMS m/z = 402.2 [M + H]⁺. 1H NMR (400 MHz, DMSO-d₆) δ: ppm |
| | 11.23 (s, 1H), 10.83 (s, 1H), 9.41 (s, 1H), 9.18 (s, 1H), 7.70 (s, 1H) , 2.87-2.93 |
| | (m, 2H), 2.15-2.29 (m, 9H), 1.29 (t, J = 7.6 Hz, 3H). |
| 171 | N-(4-((6-(difluoromethoxy)pyridin-2-yl)amino)-5-(pyrimidin-4-yl)pyridin-2- |
| | yl)acetamide |
| | 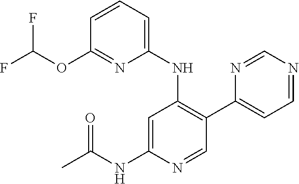 [SMILES: CC(=O)Nc1cc(Nc2cccc(OC(F)F)n2)c(-c2ccncn2)cn1] |
| | SM: N-(4-amino-5-(pyrimidin-4-yl)pyridin-2-yl)acetamide hydrochloride |
| | (Preparation 454) and 2-chloro-6-(difluoromethoxy)pyridine |
| | Prep-HPLC (Method R, Gradient 36-66%). 80.66 mg, 28.6% yield as a white |
| | solid. LCMS m/z = 373.1 [M + H]⁺ ¹H NMR (400 MHz, DMSO-d₆) δ ppm 12.47 |
| | (s, 1H), 10.69 (s, 1H), 9.34 (s, 1H), 9.14 (s, 1H), 8.91 (s, 1H), 8.88 (d, J = 5.6 |
| | Hz, 1H), 8.20-8.24 (m, 1H), 8.12 (t, J = 73.2 Hz, 1H), 7.94 (s, 1H), 7.85 (t, J = |
| | 8.0 Hz, 1H), 7.00 (d, J = 8.0 Hz, 1H), 6.64 (d, J = 7.6 Hz, 1H), 2.15 (s, 3H). |

### Example 172 to 176

The compounds in the following table were prepared from N-(4-amino-5-(2-methylpyrimidin-4-yl)pyridin-2-yl)acetamide hydrochloride (Preparation 459) and the appropriate halide, following a similar procedure to that described in Example 150.

| Example | |
|---|---|
| No | Name, Structure, Starting Materials (SM), Data |
| 172 | N-(4-((6-(1, 1-difluoroethyl)pyridin-2-yl)amino)-5-(2-methylpyrimidin-4- |
| | yl)pyridin-2-yl)acetamide |
| | 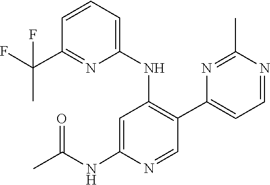 [SMILES: CC(=O)Nc1cc(Nc2cccc(C(C)(F)F)n2)c(-c2ccnc(C)n2)cn1] |
| | SM: 2-chloro-6-(1,1-difluoroethyl)pyridine |
| | Prep-HPLC (Method C, 34% to 64%). 20 mg, 12.7% yield as a yellow solid. |
| | LCMS m/z = 385.1 [M + H]⁺. 1H NMR (500 MHz, CDCl₃) δ: ppm 13.03 (s, 1H), |
| | 9.40 (s, 1H), 8.73 (d, J = 5.5 Hz, 1H), 8.62 (s, 2H), 7.76-7.79 (m, 1H), 7.57 (d, |
| | J = 5.5 Hz, 1H), 7.34 (d, J = 7.5 Hz, 1H), 7.04 (d, J = 8.5 Hz, 1H), 2.87 (s, 3H), 2.26 |
| | (s, 3H), 2.18 (t, J = 19.0 Hz, 3H). |
| 173 | N-(4-((2-(1,1-difluoroethyl)pyrimidin-4-yl)amino)-5-(2-methylpyrimidin-4- |
| | yl)pyridin-2-yl)acetamide |
| | 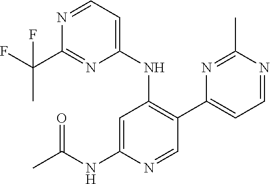 [SMILES: CC(=O)Nc1cc(Nc2ccnc(C(C)(F)F)n2)c(-c2ccnc(C)n2)cn1] |
| | SM: 4-chloro-2-(1,1-difluoroethyl)pyrimidine (Preparation 148) |
| | prep-HPLC (Method S, Gradient 25-55%) 27.6 mg, 31.0% yield as a white |
| | solid. LCMS m/z = 386.2 [M + H]⁺ ¹H NMR (400 MHz, CDCl₃) δ ppm 13.26 (s, |
| | 1H), 9.43 (s, 1H), 8.77 (d, J = 5.6 Hz, 1H), 8.73 (s, 1H), 8.62 (d, J = 6.0 Hz, |
| | 1H), 8.01 (br s, 1H), 7.63 (d, J = 5.6 Hz, 1H), 6.94 (d, J = 5.6 Hz, 1H), 2.91 (s, |
| | 3H), 2.29 (s, 3H), 2.21 (t, J = 18.8 Hz, 3H) |
| 174 | N-(4-((2-(1,1-difluoroethyl)-6-methylpyrimidin-4-yl)amino)-5-(2- |
| | methylpyrimidin-4-yl)pyridin-2-yl)acetamide |
| | 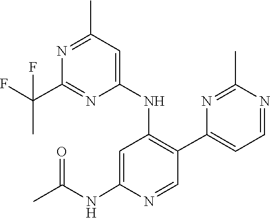 [SMILES: CC(=O)Nc1cc(Nc2cc(C)nc(C(C)(F)F)n2)c(-c2ccnc(C)n2)cn1] |
| | SM: 4-chloro-2-(1,1-difluoroethyl)-6-methylpyrimidine (Preparation 92) |
| | Prep-HPLC (Method D, 26-56%). 19 mg, 19.3% yield as a white solid. LCMS |
| | m/z= 400.2 [M + H]⁺. 1H NMR (400 MHz, CDCl₃) δ ppm 13.07 (s, 1H), 9.40 (s, |
| | 1H), 8.74 (d, J = 5.6 Hz, 1H), 8.67 (br s, 1H), 8.22 (s, 1H), 7.59 (d, J = 5.6 Hz, 1H), |
| | 6.75 (s, 1H), 2.89 (s, 3H), 2.57 (s, 3H), 2.26 (s, 3H), 2.18 (t, J = 19.2 Hz, 3H). |
| 175 | N-(4-((2-(2-fluoropropan-2-yl)pyrimidin-4-yl)amino)-5-(2-methylpyrimidin-4- |
| | yl)pyridin-2-yl)acetamide |
| | 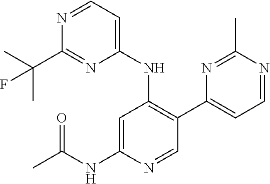 [SMILES: CC(=O)Nc1cc(Nc2ccnc(C(C)(C)F)n2)c(-c2ccnc(C)n2)cn1] |
| | SM: 4-chloro-2-(2-fluoropropan-2-yl)pyrimidine (Preparation 207) |
| | prep-HPLC (Method S, Gradient 25-55%) 51.0 mg, 46.8% yield as a white |
| | solid. LCMS m/z = 382.2 [M + H]⁺ 1H NMR (400 MHz, CDCl₃) δ ppm: 13.10 |
| | (s, 1H), 9.43 (s, 1H), 8.68-8.74 (m, 2H), 8.55 (d, J = 6.0 Hz, 1H), 8.06 (s, 1H), |
| | 7.59 (d, J = 5.6 Hz, 1H), 6.78 (d, J = 5.6 Hz, 3H), 2.84 (s, 3H), 2.23 (s, 3H), 1.82- |
| | 1.92 (m, 6H). |
| 176 | N-(4-((2-(2-fluoropropan-2-yl)-6-methylpyrimidin-4-yl)amino)-5-(2- |
| | methylpyrimidin-4-yl)pyridin-2-yl)acetamide |
| | 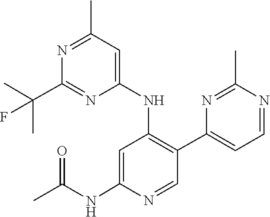 [SMILES: CC(=O)Nc1cc(Nc2cc(C)nc(C(C)(C)F)n2)c(-c2ccnc(C)n2)cn1] |
| | SM: 4-chloro-2-(2-fluoropropan-2-yl)-6-methylpyrimidine (Preparation 157) |
| | Prep-HPLC (Method S, Gradient 25-55%) 28 mg, 28.7% yield as a white solid. |
| | LCMS m/z = 396.3 [M + H]⁺ 1H NMR (400 MHz, CDCl₃) δ ppm 12.92 (s, 1H), |
| | 9.42 (s, 1H), 8.73 (d, J = 5.6 Hz, 1H), 8.66 (s, 1H), 8.03 (s, 1H), 7.58 (d, J = 5.6 |
| | Hz, 1H), 6.61 (s, 1H), 2.89 (s, 3H), 2.55 (s, 3H), 2.26 (s, 3H), 1.91 (s, 3H), 1.85 |
| | (s, 3H) |

### Examples 177 to 181

The compounds in the following table were prepared from N-(4-amino-5-(2-methoxypyrimidin-4-yl)pyridin-2-yl)acetamide hydrochloride (Preparation 457) and the appropriate halide, following a similar procedure to that described in Example 150.

| Example | |
|---|---|
| No | Name, Structure, Starting Materials (SM), Data |
| 177 | N-(4-((6-(1, 1-difluoroethyl)pyridin-2-yl)amino)-5-(2-methoxypyrimidin-4- |
| | yl)pyridin-2-yl)acetamide |
| | 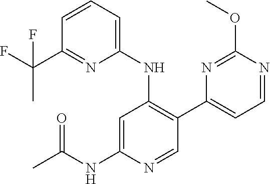 [SMILES: COc1nccc(-c2cnc(NC(C)=O)cc2Nc2cccc(C(C)(F)F)n2)n1] |
| | SM: 2-chloro-6-(1,1-difluoroethyl)pyridine |
| | HPLC (Method P: Gradient 34-64%). 90.0 mg, 29.1% yield as a white solid. |
| | LCMS m/z = 401.2 [M + H]⁺. ¹H NMR (500 MHz, CDCl₃) δ ppm: 12.55 (s, |
| | 1H), 9.28 (s, 1H), 8.59-5.60 (m, 3H), 7.76-7.80 (m, 1H), 7.32-7.37 (m, 2H), |
| | 7.15 (d, J-8.5 Hz, 1H), 4.15 (s, 3H), 2.25 (s, 3H), 2.14 (t, J = 19.0 Hz, 3H). |
| 178 | N-(4-((2-(1,1-difluoroethyl)pyrimidin-4-yl)amino)-5-(2-methoxypyrimidin-4- |
| | yl)pyridin-2-yl)acetamide |
| | 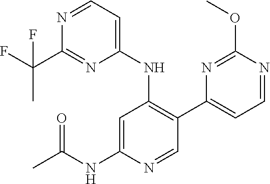 [SMILES: COc1nccc(-c2cnc(NC(C)=O)cc2Nc2ccnc(C(C)(F)F)n2)n1] |
| | SM: 4-chloro-2-(1,1-difluoroethyl)pyrimidine (Preparation 148) |
| | Prep-HPLC (Method C, Gradient 32-62%). 10 mg, 12.9% yield as a yellow |
| | solid. LCMS m/z = 402.2 [M + H]⁺ 1H NMR (400 MHz, CDCl₃) δ: ppm 12.86 |
| | (s, 1H), 9.45 (s, 1H), 8.68 (s, 1H), 8.59-8.62 (m, 2H), 8.18-8.20 (m, 1H) , 7.39 |
| | (d, J = 5.2 Hz, 1H), 7.05 (d, J = 6.0 Hz, 1H), 4.17 (s, 3H), 2.27 (s, 3H), 2.16 (t, |
| | J = 18.8 Hz, 3H). |
| 179 | N-(4-((2-(1,1-difluoroethyl)-6-methylpyrimidin-4-yl)amino)-5-(2- |
| | methoxypyrimidin-4-yl)pyridin-2-yl)acetamide |
| | 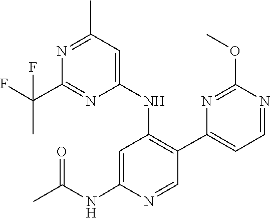 [SMILES: COc1nccc(-c2cnc(NC(C)=O)cc2Nc2cc(C)nc(C(C)(F)F)n2)n1] |
| | SM: 4-chloro-2-(1,1-difluoroethyl)-6-methylpyrimidine (Preparation 92) |
| | Prep-HPLC (Method C, 36% to 66%) 18 mg, 22.5% yield as a yellow solid. |
| | LCMS m/z = 416.2 [M + H]⁺ ¹H NMR (400 MHz, CDCl₃) δ: ppm 12.62 (s, 1H), |
| | 9.29 (s, 1H), 8.66 (s, 1H), 8.61 (d, J = 5.2 Hz, 1H), 8.07(s, 1H), 7.39 (d, J = 5.6 |
| | Hz, 1H), 6.88 (s, 1H), 4.17 (s, 3H), 2.57 (s, 3H), 2.26 (s, 3H), 2.15 (t, J = 18.8 |
| | Hz, 3H). |
| 180 | N-(4-((2-(2-fluoropropan-2-yl)pyrimidin-4-yl)amino)-5-(2-methoxypyrimidin- |
| | 4-yl)pyridin-2-yl)acetamide |
| | 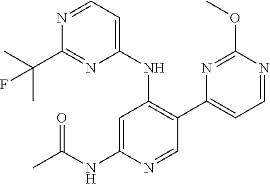 [SMILES: COc1nccc(-c2cnc(NC(C)=O)cc2Nc2ccnc(C(C)(C)F)n2)n1] |
| | SM: 4-chloro-2-(2-fluoropropan-2-yl)pyrimidine (Preparation 207) |
| | Prep-HPLC (Method R, Gradient 25-55%) 29.8 mg, 26.1% yield as a white |
| | solid. LCMS m/z = 398.2 [M + H]⁺ ¹H NMR (500 MHz, CDCl₃) δ ppm 12.62 (s, |
| | 1H), 9.33 (s, 1H), 8.67 (s, 1H), 8.60 (d, J = 5.5 Hz, 1H), 8.55 (d, J = 5.5 Hz, 1H), |
| | 7.96 (s, 1H), 7.39 (d, J = 5.5 Hz, 1H), 6.90 (d, J = 6.0 Hz, 1H), 4.16 (s, 3H), 2.26 |
| | (s, 3H), 1.89 (s, 3H), 1.84 (s, 3H) |
| 181 | N-(4-((2-(2-fluoropropan-2-yl)-6-methylpyrimidin-4-yl)amino)-5-(2- |
| | methoxypyrimidin-4-yl)pyridin-2-yl)acetamide |
| | 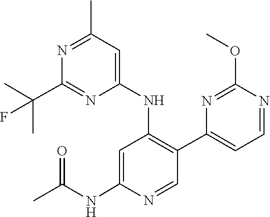 [SMILES: COc1nccc(-c2cnc(NC(C)=O)cc2Nc2cc(C)nc(C(C)(C)F)n2)n1] |
| | SM: 4-chloro-2-(2-fluoropropan-2-yl)-6-methylpyrimidine (Preparation 157) |
| | prep-HPLC (Method R, Gradient 30-60%) 21.18 mg, 25.5% yield as a white |
| | solid. LCMS m/z = 412.2 [M + H]⁺ 1H NMR (400 MHz, DMSO-d₆) δ ppm 11.59 |
| | (s, 1H), 10.65 (s, 1H), 9.15 (s, 1H), 8.82 (s, 1H), 8.68 (d, J = 5.2 Hz, 1H), 7.72 |
| | (d, J = 5.2 Hz, 1H), 6.80 (s, 1H), 4.05 (s, 3H), 2.41 (s, 3H), 2.14 (s, 3H), 1.74 (s, |
| | 3H), 1.68 (s, 3H). |

### Examples 182 to 192

The compounds in the following table were prepared from N-(4-amino-5-(5-fluoropyrimidin-2-yl)pyridin-2-yl)acetamide hydrochloride (Preparation 460) and the appropriate halide, following a similar procedure to that described in Example 150.

| Example No | Name, Structure, Starting Material (SM), Data |
|---|---|
| 182 | N-(4-((2-(1,1-difluoroethyl)-6-methylpyrimidin-4-yl)amino)-5-(5- |
| | fluoropyrimidin-2-yl)pyridin-2-yl)acetamide |
| | 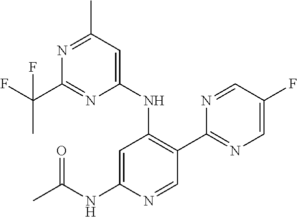 [SMILES: CC(=O)Nc1cc(Nc2cc(C)nc(C(C)(F)F)n2)c(-c2ncc(F)cn2)cn1] |
| | SM: 4-chloro-2-(1, 1-difluoroethyl)-6-methylpyrimidine (Preparation 92) |
| | Prep-HPLC (Method G, 30-60%). 106 mg, 65.0% yield, as yellow solid. |
| | LCMS m/z = 404.1 [M + H]+. 1H NMR (400 MHz, DMSO-d6) 8 ppm: 9.34 |
| | (s, 1H), 9.22 (s, 1H), 9.07 (s, 2H), 7.15 (s, 1H), 2.45 (s, 3H), 2.06-2.17 (m, |
| | 6H). |
| 183 | N-(4-((2-(1,1-difluoroethyl)-6-ethylpyrimidin-4-yl)amino)-5-(5- |
| | fluoropyrimidin-2-yl)pyridin-2-yl)acetamide |
| | 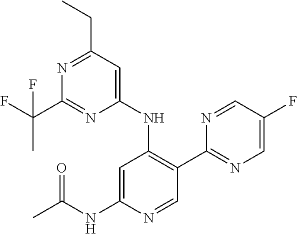 [SMILES: CCc1cc(Nc2cc(NC(C)=O)ncc2-c2ncc(F)cn2)nc(C(C)(F)F)n1] |
| | SM: 4-chloro-2-(1, 1-difluoroethyl)-6-ethylpyrimidine (Preparation 94) |
| | Prep-HPLC (Method B, 38-68%). 180 mg, 42.7% yield as a yellow solid. |
| | LCMS m/z = 418.1 [M + H]⁺. 1H NMR (500 MHz, CDCl₃) δ ppm: 12.15 (s, |
| | 1H), 9.37 (s, 1H), 9.27 (s, 1H), 8.74 (s, 2H), 8.32 (br s, 1H), 6.88 (s, 1H), |
| | 2.83 (q, J = 7.5 Hz, 2H), 2.25 (s, 3H), 2.17 (t, J = 18.8 Hz, 3H), 1.35 (t, J = 7.5 |
| | Hz, 3H) |
| 184 | N-(4-((2-(1,1-difluoroethyl)-6-isopropylpyrimidin-4-yl)amino)-5-(5- |
| | fluoropyrimidin-2-yl)pyridin-2-yl)acetamide |
| | 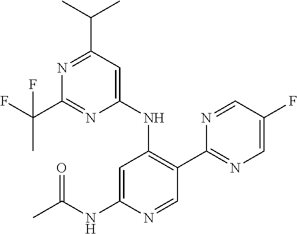 [SMILES: CC(=O)Nc1cc(Nc2cc(C(C)C)nc(C(C)(F)F)n2)c(-c2ncc(F)cn2)cn1] |
| | SM: 4-chloro-2-(1, 1-difluoroethyl)-6-isopropylpyrimidine (Preparation |
| | 142) |
| | Prep-HPLC (Method B, 40-70%). 48 mg, 23.6% yield, as a white solid. |
| | LCMS m/z = 432.1 [M + H]⁺ 1H NMR (400 MHz, MeOD-d₄) δ ppm 9.29- |
| | 9.32 (m, 2H), 8.89 (s, 2H), 7.02 (s, 1H), 2.97-3.03 (m, 1H), 2.21 (s, 3H), |
| | 2.11 (t, J = 18.8 Hz, 3H), 1.34 (d, J = 7.2 Hz, 6H). |
| 185 | N-(4-((2-(1,1-difluoroethyl)pyrimidin-4-yl)amino)-5-(5-fluoropyrimidin-2- |
| | yl)pyridin-2-yl)acetamide |
| | 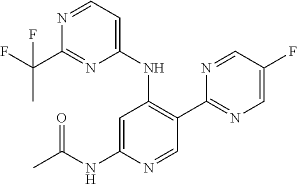 [SMILES: CC(=O)Nc1cc(Nc2ccnc(C(C)(F)F)n2)c(-c2ncc(F)cn2)cn1] |
| | SM: 4-chloro-2-(1,1-difluoroethyl)pyrimidine (Preparation 148) |
| | Prep-HPLC (Method B, 33-63%). 80 mg, 58.3% yield as a white solid. |
| | LCMS m/z = 390.1 [M + H]⁺. ¹H NMR (400 MHz, DMSO-d₆) 8 ppm: 12.30 |
| | (s, 1H), 10.67 (s, 1H), 9.32 (s, 1H), 9.22 (s, 1H), 9.08 (s, 2H), 8.62 (d, J = 6.0 |
| | Hz, 1H), 7.29 (d, J = 6.0 Hz, 1H), 2.06-2.16 (m, 6H) |
| 186 | N-(4-((6-(1, 1-difluoroethyl)pyridin-2-yl)amino)-5-(5-fluoropyrimidin-2- |
| | yl)pyridin-2-yl)acetamide |
| | 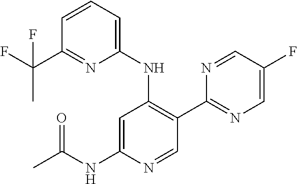 [SMILES: CC(=O)Nc1cc(Nc2cccc(C(C)(F)F)n2)c(-c2ncc(F)cn2)cn1] |
| | SM: 2-bromo-6-(1,1-difluoroethyl)pyridine |
| | Prep-HPLC (Method B, 37-65%). 63 mg, 32.9% yield, as white solid. |
| | LCMS m/z = 389.0 [M + H]⁺. ¹H NMR: (500 MHz, CDCl₃) δ ppm: 12.00 (s, |
| | 1H), 9.34 (s, 1H), 9.23 (s, 1H), 8.71 (s, 2H), 8.36 (s, 1H), 7.74 (t, J = 8.0 Hz, |
| | 1H), 7.29-7.31 (m, 1H), 7.10 (d, J = 8.0 Hz, 1H), 2.13-2.23 (m, 6H). |
| 187 | N-(4-((2-(1, 1-difluoroethyl)pyridin-4-yl)amino)-5-(5-fluoropyrimidin-2- |
| | yl)pyridin-2-yl)acetamide |
| | 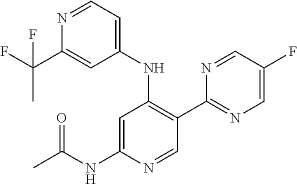 [SMILES: CC(=O)Nc1cc(Nc2ccnc(C(C)(F)F)c2)c(-c2ncc(F)cn2)cn1] |
| | SM: 4-chloro-2-(1, 1-difluoroethyl)pyridine |
| | Prep-HPLC (Method B, 34-64%). 16.9 mg, 17.9% yield as a white solid. |
| | LCMS m/z = 389.2 [M + H]⁺ ¹H NMR (400 MHz, CDCl₃) δ ppm: 11.49 (s, |
| | 1H), 9.33 (s, 1H), 8.73 (s, 2H), 8.58 (s, 1H), 8.45 (s, 1H), 8.32 (m, 1H), 7.49 |
| | (s, 1H), 7.43 (s, 1H), 2.24 (s, 3H), 2.01-2.11 (t, J = 12.5 Hz, 3H). |
| 188 | N-(4-((6-(1, 1-difluoroethyl)pyrazin-2-yl)amino)-5-(5-fluoropyrimidin-2- |
| | yl)pyridin-2-yl)acetamide |
| | 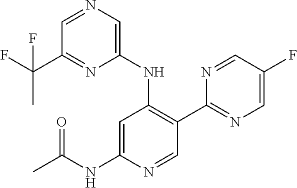 [SMILES: CC(=O)Nc1cc(Nc2cncc(C(C)(F)F)n2)c(-c2ncc(F)cn2)cn1] |
| | SM: 2-chloro-6-(1, 1-difluoroethyl)pyrazine |
| | Prep-HPLC (Method B, 35-65%). 27.0 mg, 26.3% yield as a white solid. |
| | LCMS m/z = 390.1 [M + H]⁺ ¹H NMR (500 MHz, CDCl₃) δ ppm: 12.61 (s, |
| | 1H), 9.54 (s, 1H), 9.40 (s, 1H), 8.76 (s, 2H), 8.59 (s, 1H), 8.53 (s, 1H), 8.44 |
| | (s, 1H), 2.20-2.28 (m, 6H). |
| 189 | N-(4-((6-(2-fluoropropan-2-yl)pyrazin-2-yl)amino)-5-(5-fluoropyrimidin- |
| | 2-yl)pyridin-2-yl)acetamide |
| | 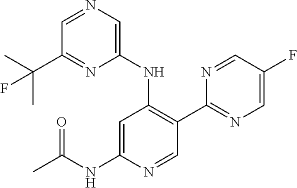 [SMILES: CC(=O)Nc1cc(Nc2cncc(C(C)(C)F)n2)c(-c2ncc(F)cn2)cn1] |
| | SM: 2-chloro-6-(2-fluoropropan-2-yl)pyrazine |
| | Prep-HPLC (Method B, 30-60%). 10.0 mg, 8.0% yield as a white solid. |
| | LCMS m/z = 386.1 [M + H]⁺ ¹H NMR: (400 MHz, CDCl₃) δ ppm: 12.48 (s, |
| | 1H), 9.56 (s, 1H), 9.39 (s, 1H), 8.75 (s, 2H), 8.48 (br s, 1H), 8.44 (s, 1H), |
| | 8.28 (s, 1H), 2.24 (s, 3H), 1.90 (s, 3H), 1.85 (s, 3H). |
| 190 | N-(4-((6-(1, 1-difluoroethyl)-4-methylpyridin-2-yl)amino)-5-(5- |
| | fluoropyrimidin-2-yl)pyridin-2-yl)acetamide |
| | 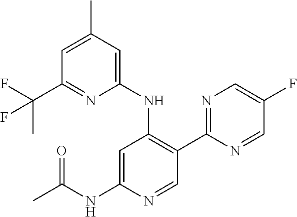 [SMILES: CC(=O)Nc1cc(Nc2cc(C)cc(C(C)(F)F)n2)c(-c2ncc(F)cn2)cn1] |
| | SM: 2-chloro-6-(1,1-difluoroethyl)-4-methylpyridine (Preparation 128) |
| | Prep-HPLC (Method B, 46-66%). 73.0 mg, 57.2% yield as a white solid. |
| | LCMS m/z = 403.1 [M + H]⁺ ¹H NMR (400 MHz, CDCl₃) δ ppm: 11.98 (s, |
| | 1H), 9.32 (s, 1H), 9.26 (s, 1H), 8.78 (s, 2H), 8.48 (br s, 1H), 7.16 (s, 1H), |
| | 6.91 (s, 1H), 2.42 (s, 3H), 2.11-2.23 (m, 6H). |
| 191 | N-(4-((6-(1,1-difluoroethyl)-4-methoxypyridin-2-yl)amino)-5-(5- |
| | fluoropyrimidin-2-yl)pyridin-2-yl)acetamide |
| | 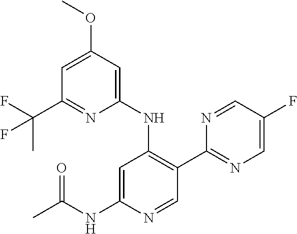 [SMILES: COc1cc(Nc2cc(NC(C)=O)ncc2-c2ncc(F)cn2)nc(C(C)(F)F)c1] |
| | SM: 2-bromo-6-(1, 1-difluoroethyl)-4-methoxypyridine (Preparation 131) |
| | Prep-HPLC (Method B, 42-72%). 8.52 mg, 12.6% yield as a white solid. |
| | LCMS m/z = 419.1 [M + H]⁺ ¹H NMR (500 MHz, CDCl₃) δ ppm: 11.96 (s, |
| | 1H), 9.31 (s, 1H), 9.13 (s, 1H), 8.73 (s, 2H), 6.96 (s, 1H), 6.66 (s, 1H), 3.94 |
| | (s, 3H), 2.24 (s, 3H), 2.12 (t, J = 19.0 Hz, 3H). |
| 192 | N-(4-((2-(1,1-difluoroethyl)-6-methoxypyrimidin-4-yl)amino)-5-(5- |
| | fluoropyrimidin-2-yl)pyridin-2-yl)acetamide |
| | 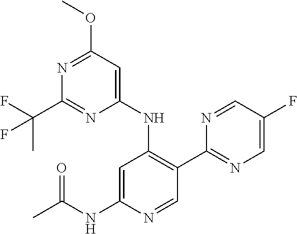 [SMILES: COc1cc(Nc2cc(NC(C)=O)ncc2-c2ncc(F)cn2)nc(C(C)(F)F)n1] |
| | SM: 4-chloro-2-(1,1-difluoroethyl)-6-methoxypyrimidine (Preparation 97) |
| | Prep-HPLC (Method B, 35-65%). 62.3 mg, 42.1% yield as a white solid. |
| | LCMS m/z = 420.1 [M + H]⁺ ¹H NMR (400 MHz, CDCl₃) δ ppm: 12.09 (s, |
| | 1H), 9.35 (s, 1H), 9.13 (s, 1H), 8.75 (s, 2H), 8.44 (br s, 1H), 6.50 (s, 1H), |
| | 4.05 (s, 3H), 2.27 (s, 3H), 2.13 (t, J = 18.8 Hz, 3H). |

### Example 193 to 196

The compounds in the following table were prepared from N-(4-amino-5-(6-methoxypyrimidin-4-yl)pyridin-2-yl)acetamide hydrochloride (Preparation 458) and the appropriate halide, following a similar procedure to that described in Example 150.

| Example No | Name, Structure, Starting material (SM), Data |
|---|---|
| 193 | N-(4-((2-(1,1-difluoroethyl)pyrimidin-4-yl)amino)-5-(6-methoxypyrimidin- |
| | 4-yl)pyridin-2-yl)acetamide |
| | 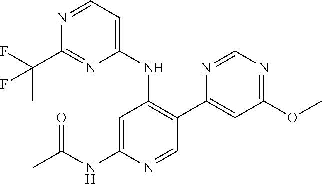 [SMILES: COc1cc(-c2cnc(NC(C)=O)cc2Nc2ccnc(C(C)(F)F)n2)ncn1] |
| | SM: 4-chloro-2-(1,1-difluoroethyl)pyrimidine (Preparation 148) |
| | prep-HPLC (Method Y, Gradient 22-52%). 16.8 mg, 21.70% yield as a |
| | yellow solid. LCMS m/z = 402.0 [M + H]^{+.} ¹H NMR (400 MHz, CDCl₃) δ ppm: |
| | 12.61 (s, 1H), 9.36 (s, 1H), 8.88 (s, 1H), 8.50-8.64 (m, 2H), 8.50-8.64 (m, |
| | 2H), 8.08 (s, 1H), 7.13 (s, 1H), 6.93 (d, J = 5.6 Hz, 1H), 4.07 (s, 3H), 2.26 (s, |
| | 3H), 2.18 (t, J = 18.8 Hz, 3H). |
| 194 | N-(4-((2-(1,1-difluoroethyl)-6-methylpyrimidin-4-yl)amino)-5-(6- |
| | methoxypyrimidin-4-yl)pyridin-2-yl)acetamide |
| | 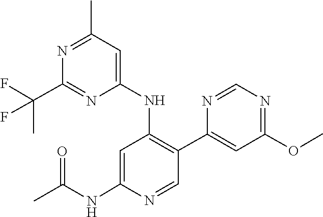 [SMILES: COc1cc(-c2cnc(NC(C)=O)cc2Nc2cc(C)nc(C(C)(F)F)n2)ncn1] |
| | SM: 4-chloro-2-(1,1-difluoroethyl)-6-methylpyrimidine (Preparation 92) |
| | Prep-HPLC (Method R, Gradient 33-53%). 9.1 mg, 5.7% yield as a white |
| | solid. LCMS m/z = 416.2 [M + H]⁺. ¹H NMR (400 MHz, DMSO-d₆) δ ppm: |
| | 12.29 (s, 1H), 10.62 (s, 1H), 9.23 (s, 1H), 8.96 (s, 1H), 8.83 (s, 1H), 7.55 (s, |
| | 1H), 7.08 (s, 1H), 4.00 (s, 3H), 2.44 (s, 3H), 2.10-2.16 (m, 6H). |
| 195 | N-(4-((2-(2-fluoropropan-2-yl)pyrimidin-4-yl)amino)-5-(6- |
| | methoxypyrimidin-4-yl)pyridin-2-yl)acetamide |
| | 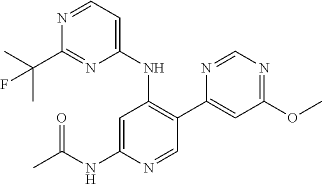 [SMILES: COc1cc(-c2cnc(NC(C)=O)cc2Nc2ccnc(C(C)(C)F)n2)ncn1] |
| | SM: 4-chloro-2-(2-fluoropropan-2-yl)pyrimidine (Preparation 207) |
| | Prep-HPLC (Method D, 18-48%). 7.2 mg, 6.70% yield as a white solid. |
| | LCMS m/z = 398.2 [M + H]⁺. ¹H NMR (500 MHz, CDCl₃) δ ppm: 12.47 (s, |
| | 1H), 9.41 (s, 1H), 8.87 (s, 1H), 8.56 (s, 1H), 8.51 (d, J-6.0 Hz, 1H), 8.02 (s, |
| | 1H), 7.12 (s, 1H), 6.78 (d, J = 5.5 Hz, 1H), 4.06 (s, 3H), 2.25 (s, 3H), 1.90 (s, |
| | 3H), 1.86 (s, 3H). |
| 196 | N-(4-((2-(2-fluoropropan-2-yl)-6-methylpyrimidin-4-yl)amino)-5-(6- |
| | methoxypyrimidin-4-yl)pyridin-2-yl)acetamide |
| | 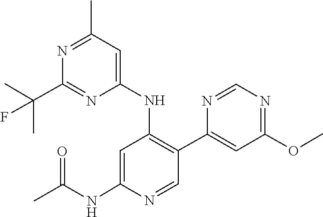 [SMILES: COc1cc(-c2cnc(NC(C)=O)cc2Nc2cc(C)nc(C(C)(C)F)n2)ncn1] |
| | SM: 4-chloro-2-(1, 1-difluoroethyl)-6-methylpyrimidine (Preparation 92) |
| | Prep-HPLC (Method B, 37-67%). 36 mg, 32.3% yield) as a white solid. |
| | LCMS m/z = 412.1 [M + H]⁺. ¹H NMR: (400 MHz, CDCl₃) δ ppm: 12.35 (s, |
| | 1H), 9.42 (s, 1H), 8.88 (s, 1H), 8.54 (s, 1H), 8.08 (s, 1H), 7.11 (s, 1H), 6.62 |
| | (s, 1H), 4.06 (s, 3H), 2.53 (s, 3H), 2.24 (s, 3H), 1.90 (s, 3H), 1.85 (s, 3H). |

### Examples 197 to X

The compounds in the following table were prepared from N-(4-amino-5-(6-methylpyrimidin-4-yl)pyridin-2-yl)acetamide hydrochloride (Preparation 461) and the appropriate halide, following a similar procedure to that described in Example 150.

| Example No | Name, Structure, Starting Material (SM), Data |
|---|---|
| 197 | N-(4-((2-(1,1-difluoroethyl)-6-methylpyrimidin-4-yl)amino)-5-(6- |
| | methylpyrimidin-4-yl)pyridin-2-yl)acetamide |
| | 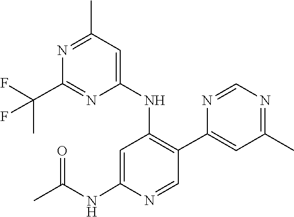 [SMILES: CC(=O)Nc1cc(Nc2cc(C)nc(C(C)(F)F)n2)c(-c2cc(C)ncn2)cn1] |
| | SM: 4-chloro-2-(1,1-difluoroethyl)-6-methylpyrimidine (Preparation 92) |
| | Prep-HPLC (Method B, 21-51%). 35.1 mg, 49.2% yield as a white solid. |
| | LCMS m/z = 400.1 [M + H]⁺. ¹H NMR (500 MHz, CDCl₃) δ ppm: 12.72 (s, |
| | 1H), 9.47 (s, 1H), 9.18 (s, 1H), 8.64 (s, 1H), 8.38 (br s, 1H), 7.63 (s, 1H), 6.80 |
| | (s, 1H), 2.65 (s, 3H), 2.57 (s, 3H), 2.27 (s, 3H), 2.18 (t, J = 19.0 Hz, 3H). |
| 198 | N-(4-((2-(2-fluoropropan-2-yl)pyrimidin-4-yl)amino)-5-(6- |
| | methylpyrimidin-4-yl)pyridin-2-yl)acetamide |
| | 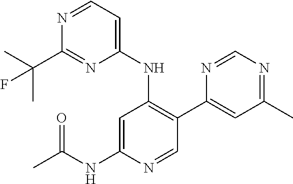 [SMILES: CC(=O)Nc1cc(Nc2ccnc(C(C)(C)F)n2)c(-c2cc(C)ncn2)cn1] |
| | SM: 4-chloro-2-(2-fluoropropan-2-yl)pyrimidine (Preparation 207) |
| | Prep-HPLC (Method R, Gradient 25-55%) |
| | 28.0 mg, 41.5% yield as yellow solid. LCMS m/z = 382.2 [M + H]⁺ |
| | ¹H NMR (400 MHz, CDCl₃) δ ppm: 12.64 (s, 1H), 9.46 (s, 1H), 9.16 (s, 1H), |
| | 8.65 (s, 1H), 8.53 (d, J = 6.0 Hz, 1H), 8.08 (br s, 1H), 7.64 (s, 1H), 6.80 (d, |
| | J = 6.0 Hz, 1H), 2.65 (s, 3H), 2.26 (s, 3H), 1.91 (s, 3H), 1.86 (s, 3H). |
| 199 | N-(4-((2-(1,1-difluoroethyl)pyridin-4-yl)amino)-5-(6-methylpyrimidin-4- |
| | yl)pyridin-2-yl)acetamide |
| | 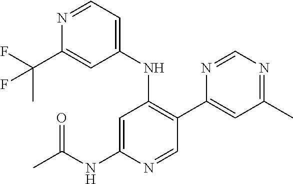 [SMILES: CC(=O)Nc1cc(Nc2ccnc(C(C)(F)F)c2)c(-c2cc(C)ncn2)cn1] |
| | SM: 4-chloro-2-(1,1-difluoroethyl)pyridine |
| | Prep-HPLC (Method R, Gradient 21-51%) and Prep-HPLC (Method D, 16-46%) |
| | 20.8 mg, 18.9% yield as a yellow solid. LCMS m/z = 385.2 [M + H]⁺ |
| | ¹H NMR (400 MHz, CDCl₃) δ ppm 11.87 (s, 1H), 9.15 (s, 1H), 8.63 (s, 1H), |
| | 8.56 (d, J = 5.6 Hz, 1H), 8.38-8.47 (m, 2H), 7.62 (s, 1H), 7.49 (d, J = 1.6 Hz, |
| | 1H), 7.34-7.40 (m, 1H), 2.64 (s, 3H), 2.24 (s, 3H), 2.05 (t, J = 18.8 Hz, 3H). |
| 200 | N-(4-((2-(2-fluoropropan-2-yl)-6-methylpyrimidin-4-yl)amino)-5-(6- |
| | methylpyrimidin-4-yl)pyridin-2-yl)acetamide |
| | 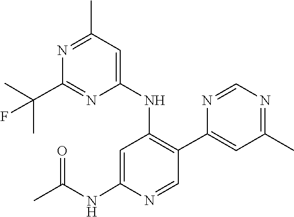 [SMILES: CC(=O)Nc1cc(Nc2cc(C)nc(C(C)(C)F)n2)c(-c2cc(C)ncn2)cn1] |
| | SM: 4-chloro-2-(2-fluoropropan-2-yl)-6-methylpyrimidine (Preparation 157) |
| | Prep-HPLC (Method D, 14-42%). 41 mg, 36.3% yield as yellow solid. LCMS |
| | m/z = 396.2 [M + H]⁺ 1H NMR (500 MHz, DMSO-d₆) δ ppm: 12.44 (s, 1H), |
| | 10.62 (s, 1H), 9.36 (s, 1H), 9.19 (s, 1H), 8.88 (s, 1H), 8.11 (s, 1H), 6.95 (s, |
| | 1H), 2.54 (s, 3H), 2.41 (s, 3H), 2.13 (s, 3H), 1.76 (s, 3H), 1.72 (s, 3H). |
| 201 | N-(4-((2-(1,1-difluoroethyl)pyrimidin-4-yl)amino)-5-(6-methylpyrimidin-4- |
| | yl)pyridin-2-yl)acetamide |
| | 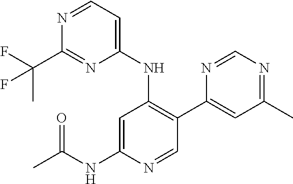 [SMILES: CC(=O)Nc1cc(Nc2ccnc(C(C)(F)F)n2)c(-c2cc(C)ncn2)cn1] |
| | SM: 4-chloro-2-(1,1-difluoroethyl)pyrimidine (Preparation 148) |
| | prep-HPLC (Method R, Gradient 22-52%). 22.1 mg, 53.5% yield as a white |
| | solid. LCMS m/z = 386.1 [M + H]⁺. ¹H NMR (500 MHz, CDCl₃) δ ppm: 12.85 |
| | (s, 1H), 9.48 (s, 1H), 9.17 (s, 1H), 8.66 (s, 1H), 8.59 (d, J = 5.5 Hz, 1H), 8.34 |
| | (br s, 1H), 7.65 (s, 1H), 6.96 (d, J = 5.5 Hz, 1H), 2.66 (s, 3H), 2.27 (s, 3H), |
| | 2.18 (t, J = 19.0 Hz, 3H). |

### Examples 202 to 212

The compounds in the following table were prepared from N-(4-amino-5-(1-methyl-1H-pyrazol-3-yl)pyridin-2-yl)acetamide (Preparation 481) and the appropriate halide, following a similar procedure to that described in Example 150.

| Example No | Name, Structure, Starting Material (SM), Data |
|---|---|
| 202 | N-(4-((2-(1,1-difluoroethyl)pyrimidin-4-yl)amino)-5-(1-methyl-1H-pyrazol- |
| | 3-yl)pyridin-2-yl)acetamide |
| | 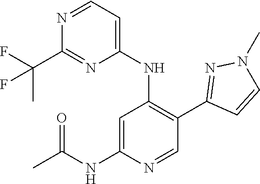 [SMILES: CC(=O)Nc1cc(Nc2ccnc(C(C)(F)F)n2)c(-c2ccn(C)n2)cn1] |
| | SM: 4-chloro-2-(1,1-difluoroethyl)pyrimidine (Preparation 148) |
| | prep-HPLC (Method K, Gradient 32-62%) . 15 mg, 11.6% yield as white |
| | solid. LCMS m/z = 374.1 [M + H]⁺ ¹H NMR (500 MHz, CDCl₃) δ ppm: 11.56 |
| | (s, 1H), 9.43 (s, 1H), 8.55 (d, J = 5.5 Hz, 1H), 8.48 (s, 1H), 8.33 (s, 1H), 7.47 |
| | (d, J = 2.5 Hz, 1H), 6.93 (d, J = 5.5 Hz, 1H), 6.64 (d, J = 2.5 Hz, 1H), 4.04 (s, |
| | 3H), 2.16-2.26 (m, 6H). |
| 203 | N-(4-((4-(1,1-difluoroethyl)pyrimidin-2-yl)amino)-5-(1-methyl-1H-pyrazol- |
| | 3-yl)pyridin-2-yl)acetamide |
| | 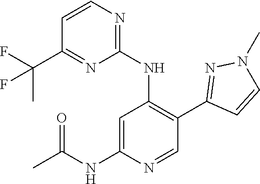 [SMILES: CC(=O)Nc1cc(Nc2nccc(C(C)(F)F)n2)c(-c2ccn(C)n2)cn1] |
| | SM: 2-chloro-4-(1,1-difluoroethyl)pyrimidine |
| | prep-HPLC (Method P, Gradient 35-65%). 20 mg, 12.7% yield as white |
| | solid. LCMS m/z = 374.2 [M + H]⁺. ¹H NMR (400 MHz, CDCl₃) δ ppm: 11.62 |
| | (s, 1H), 9.59 (s, 1H), 8.70 (d, J = 4.8 Hz, 1H), 8.45 (s, 1H), 8.26 (s, 1H), 7.45 |
| | (d, J = 2.0 Hz, 1H), 7.16 (d, J = 5.2 Hz, 1H), 6.62 (d, J = 2.0 Hz, 1H), 4.05 (s, |
| | 3H), 2.11-2.24 (m, 6H). |
| 204 | N-(4-((2-(1,1-difluoroethyl)-6-ethylpyrimidin-4-yl)amino)-5-(1-methyl-1H- |
| | pyrazol-3-yl)pyridin-2-yl)acetamide |
| | 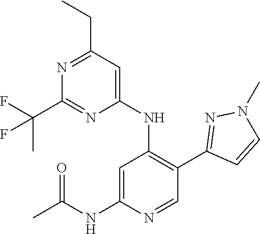 [SMILES: CCc1cc(Nc2cc(NC(C)=O)ncc2-c2ccn(C)n2)nc(C(C)(F)F)n1] |
| | SM: 4-chloro-2-(1,1-difluoroethyl)-6-ethylpyrimidine (Preparation 94) |
| | prep-HPLC (Method S, Gradient 33-63%) 75.4 mg, 57.9% yield as white |
| | solid. LCMS m/z = 402.2 [M + H]⁺ ¹H NMR (400 MHz, CDCl₃) δ ppm: 11.48 |
| | (s, 1H), 9.46 (s, 1H), 8.75 (br s, 1H), 8.43 (s, 1H), 7.49 (d, J = 2.4 Hz, 1H), |
| | 6.80 (s, 1H), 6.64 (d, J = 2.4 Hz, 1H), 4.07 (s, 3H), 2.84 (q, J = 7.6 Hz, 2H), |
| | 2.15-2.28 (m, 6H), 1.36 (t, J = 7.6 Hz, 3H). |
| 205 | N-(4-((2-(1,1-difluoroethyl)-6-isopropylpyrimidin-4-yl)amino)-5-(1-methyl- |
| | 1H-pyrazol-3-yl)pyridin-2-yl)acetamide |
| | 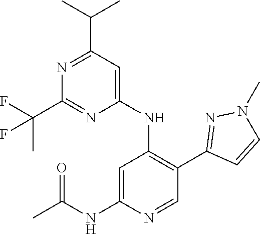 [SMILES: CC(=O)Nc1cc(Nc2cc(C(C)C)nc(C(C)(F)F)n2)c(-c2ccn(C)n2)cn1] |
| | SM: 4-chloro-2-(1,1-difluoroethyl)-6-isopropylpyrimidine (Preparation 142) |
| | HPLC (Method O, Gradient 35-65%). 55.0 mg, 23.6% yield as a white solid. |
| | LCMS m/z = 416.3 [M + H]⁺. ¹H NMR (400 MHz, CDCl₃) δ ppm: 11.25 (s, |
| | 1H), 9.28 (s, 1H), 8.47 (s, 1H), 8.15 (s, 1H), 7.46 (d, J = 2.4 Hz, 1H), 6.80 (s, |
| | 1H), 6.63 (d, J = 2.0 Hz, 1H), 4.03 (s, 3 H), 2.98-3.09 (m, 1H), 2.13-2.23 (m, |
| | 6H), 1.33 (d, J = 6.8 Hz, 6H). |
| 206 | N-(4-((6-cyclopropyl-2-(1,1-difluoroethyl)pyrimidin-4-yl)amino)-5-(1- |
| | methyl-1H-pyrazol-3-yl)pyridin-2-yl)acetamide |
| | 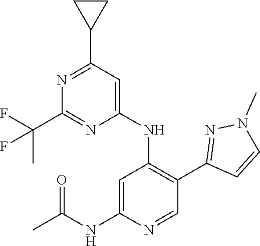 [SMILES: CC(=O)Nc1cc(Nc2cc(C3CC3)nc(C(C)(F)F)n2)c(-c2ccn(C)n2)cn1] |
| | SM: 4-chloro-6-cyclopropyl-2-(1,1-difluoroethyl)pyrimidine (Preparation 155) |
| | Prep-HPLC (Method B, 40-70%) |
| | 23.5 mg, 16.4% yield) as a white solid. LCMS m/z = 414.1 [M + H]⁺ |
| | ¹H NMR (400 MHz, CDCl₃) δ ppm: 11.19 (s, 1H), 9.24 (s, 1H), 8.45 (s, 1H), |
| | 8.06 (s, 1H), 7.45 (d, J = 2.4 Hz, 1H), 6.79 (s, 1H), 6.62 (d, J = 2.4 Hz, 1H), 4.03 |
| | (s, 3H), 2.23 (s, 3H), 2.12 (t, J = 18.8 Hz, 3H), 1.99-2.02 (m, 1H), 1.19-1.21 (m, |
| | 2H), 1.05-1.08 (m, 2H). |
| 207 | N-(4-((2-(1,1-difluoroethyl)-6-(2-methoxyethoxy)pyrimidin-4-yl)amino)-5- |
| | (1-methyl-1H-pyrazol-3-yl)pyridin-2-yl)acetamide |
| | 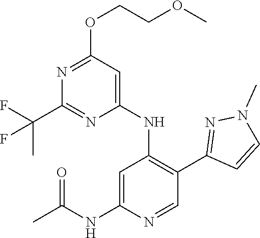 [SMILES: COCCOc1cc(Nc2cc(NC(C)=O)ncc2-c2ccn(C)n2)nc(C(C)(F)F)n1] |
| | SM: 4-chloro-2-(1,1-difluoroethyl)-6-(2-methoxyethoxy)pyrimidine |
| | (Preparation 158) |
| | prep-HPLC (Method P, Gradient 33-63%). 61.0 mg, 41.4% yield as a white |
| | solid. LCMS m/z = 448.2 [M + H]⁺. ¹H NMR (400 MHz, CDCl₃) δ ppm: 11.42 |
| | (s, 1H), 9.38 (s, 1H), 8.38-8.43 (m, 2H), 7.45 (d, J = 2.0 Hz, 1H), 6.62 (d, J = 2.4 |
| | Hz, 1H), 6.36 (s, 1H), 4.60-4.63 (m, 2H), 4.02 (s, 3H), 3.74-3.77 (m, 2H), 3.45 |
| | (s, 3H), 2.11-2.25 (m, 6H). |
| 208 | N-(5-(1-methyl-1H-pyrazol-3-yl)-4-((6-(tetrahydrofuran-3-yl)pyridin-2- |
| | yl)amino)pyridin-2-yl)acetamide |
| | 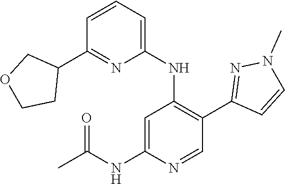 [SMILES: CC(=O)Nc1cc(Nc2cccc(C3CCOC3)n2)c(-c2ccn(C)n2)cn1] |
| | SM: 2-bromo-6-(tetrahydrofuran-3-yl)pyridine |
| | prep-HPLC (Method S, Gradient 26-56%) 26.2 mg, 21.4% yield as a white |
| | solid. LCMS m/z = 379.2 [M + H]⁺ ¹H NMR (400 MHz, CDCl₃) δ ppm: 10.91 |
| | (s, 1H), 9.28 (s, 1H), 8.40 (s, 1H), 8.15 (s, 1H), 7.53-7.58 (m, 1H), 7.43 (d, |
| | J = 2.4 Hz, 1H), 6.61-6.88 (m, 2H), 6.61 (d, J = 2.4 Hz, 1H), 4.21-4.26 (m, 1H), |
| | 4.10-4.15 (m, 1H), 4.01 (s, 3H), 3.95-4.00 (m, 2H), 3.60-3.62 (m, 1H), 2.38- |
| | 2.43 (m, 2H), 2.22 (s, 3H). |
| 209 | N-(4-((6-(1,1-difluoroethyl)-4-methoxypyridin-2-yl)amino)-5-(1-methyl-1H- |
| | pyrazol-3-yl)pyridin-2-yl)acetamide |
| | 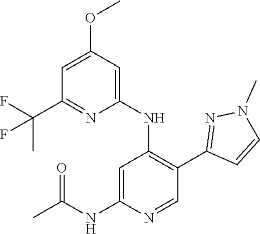 [SMILES: COc1cc(Nc2cc(NC(C)=O)ncc2-c2ccn(C)n2)nc(C(C)(F)F)c1] |
| | SM: 2-bromo-6-(1,1-difluoroethyl)-4-methoxypyridine (Preparation 131) |
| | prep-HPLC (Method P, Gradient 36-66%). 60.0 mg, 57.5% yield as a white |
| | solid. ¹H NMR (400 MHz, CDCl₃) δ ppm: 10.97 (s, 1H), 9.16 (s, 1H), 8.40 (s, |
| | 1H), 8.20 (s, 1H), 7.44 (s, 1H), 6.88 (s, 1H), 6.57-6.62 (m, 2H), 4.01 (s, 3H), |
| | 3.93 (s, 3H), 2.09-2.22 (m, 6H). |
| 210 | N-(4-((6-(1,1-difluoroethyl)-4-(2-methoxyethoxy)pyridin-2-yl)amino)-5-(1- |
| | methyl-1H-pyrazol-3-yl)pyridin-2-yl)acetamide |
| | 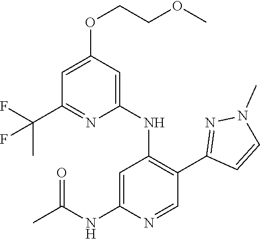 [SMILES: COCCOc1cc(Nc2cc(NC(C)=O)ncc2-c2ccn(C)n2)nc(C(C)(F)F)c1] |
| | SM: 2-chloro-6-(1,1-difluoroethyl)-4-(2-methoxyethoxy)pyridine |
| | (Preparation 161) |
| | HPLC( Method N, Gradient 28-58%). 26.3 mg, 16.1% yield as a white solid. |
| | LCMS m/z = 447.1 [M + H]⁺ 1H NMR (400 MHz, DMSO-d₆) δ ppm 11.13 (s, |
| | 1H), 10.32 (s, 1H), 9.21 (s, 1H), 8.59 (s, 1H), 7.87 (d, J = 2.4 Hz, 1H), 6.90 (d, |
| | J = 2.4 Hz, 1H), 6.83 (d, J = 1.6 Hz, 1H), 6.68 (d, J = 1.2 Hz, 1H), 4.26-4.33 (m, |
| | 2H), 4.02 (s, 3H), 3.66-3.71 (m, 2H), 3.32 (s, 3H), 2.07-2.17 (m, 6H). |
| 211 | N-(4-((6-(1,1-difluoroethyl)-4-((1s,3s)-3-methoxycyclobutoxy)pyridin-2- |
| | yl)amino)-5-(1-methyl-1H-pyrazol-3-yl)pyridin-2-yl)acetamide |
| | 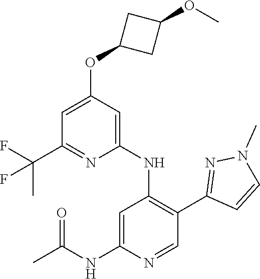 [SMILES: CO[C@H]1C[C@@H](Oc2cc(Nc3cc(NC(C)=O)ncc3-c3ccn(C)n3)nc(C(C)(F)F)c2)C1] |
| | SM: 2-chloro-6-(1,1-difluoroethyl)-4-((1s,3s)-3- |
| | methoxycyclobutoxy)pyridine (Preparation 89) |
| | prep-HPLC (Method S, Gradient 26-56%) 48.5 mg, 31.7% yield as a white |
| | solid. LCMS m/z = 473.2 [M + H]⁺ ¹H NMR (400 MHz, CDCl₃) δ ppm: 10.98 |
| | (s, 1H), 9.17 (s, 1H), 8.40 (s, 1H), 8.13 (s, 1H), 7.44 (s, 1H), 6.78 (s, 1H), 6.61 |
| | (d, J = 2.4 Hz, 1H), 6.45 (s, 1H), 4.46-4.52 (m, 1H), 4.01 (s, 3H), 3.67-3.70 (m, |
| | 1H), 3.28 (s, 3H), 2.89-2.95 (m, 2H), 2.09-2.21 (m, 8H) |
| 212 | N-(4-((6-(1,1-difluoroethyl)pyrazin-2-yl)amino)-5-(1-methyl-1H-pyrazol-3- |
| | yl)pyridin-2-yl)acetamide |
| | 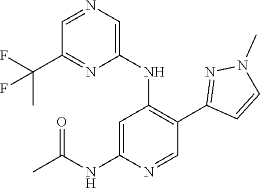 [SMILES: CC(=O)Nc1cc(Nc2cncc(C(C)(F)F)n2)c(-c2ccn(C)n2)cn1] |
| | SM: 2-chloro-6-(1,1-difluoroethyl)pyrazine |
| | prep-HPLC (Method P, Gradient 28-58%). 57.8 mg, 38.1% yield as a light- |
| | yellow solid. LCMS m/z = 374.1 [M + H]⁺ 1H NMR (400 MHz, CDCl₃) δ ppm |
| | 12.12 (s, 1H), 10.34 (br s, 1H), 9.73 (s, 1H), 8.59 (s, 1H), 8.45 (s, 1H), 8.35 |
| | (s, 1H), 7.51-7.55 (m, 1H), 6.65 (d, J = 2.4 Hz, 1H), 4.07 (s, 3H), 2.19-2.32 (m, 6H). |

### Example 213

### N-(4-((2-(1,1-difluoroethyl)-6-methylpyrimidin-4-yl)amino)-5-(6-methoxypyridazin-3-yl)pyridin-2-yl)acetamide-2,2,2-d3

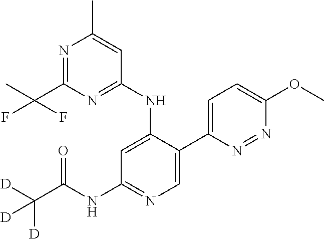 [SMILES: [2H]C([2H])([2H])C(=O)Nc1cc(Nc2cc(C)nc(C(C)(F)F)n2)c(-c2ccc(OC)nn2)cn1]

A vial containing 4-chloro-2-(1,1-difluoroethyl)-6-methylpyrimidine (Preparation 92, 48 mg, 0.249 mmol), N-(4-amino-5-(6-methoxypyridazin-3-yl)pyridin-2-yl)acetamide-2,2,2-d3 trifluoroacetate (Preparation 473, 61 mg, 0.162 mmol), K₃PO₄ (121 mg, 0.572 mmol), Xantphos (33 mg, 57 umol), DIPEA (0.2 mL, 1.15 mmol), and Pd₂dba₃ (23 mg, 25 umol) in dioxane (2 mL) was degassed then backfilled with N₂ then heated at 95° C. for 1.5 h. The cooled mixture was loaded onto a silica gel column and purified eluting with (25-85% 3:1 EtOAc: EtOH in heptane). The product was further purified by HPLC (Method U, Gradient 5-600%), to give N-(4-((2-(1,1-difluoroethyl)-6-methylpyrimidin-4-yl)amino)-5-(6-methoxypyridazin-3-yl)pyridin-2-yl)acetamide-2,2,2-d3 (7 mg, 10 00 yield) as a white solid. LCMS m/z=419.1 [M+H]⁺. ¹H NMR (500 MHz, DMSO-d₆) δ (ppm)=11.54 (br s, 1H), 10.62 (br s, 1H), 9.08 (br s, 1H), 8.68 (br s, 1H), 8.22 (dd, J=2.3, 9.3 Hz, 1H), 7.40 (br d, J=8.9 Hz, 1H), 6.95 (br s, 1H), 4.10 (d, J=2.7 Hz, 3H), 2.42 (br s, 3H), 2.04 (br t, J=19.2 Hz, 3H).

### Example 214

### N-(4-((2-(1,1-difluoroethyl)pyrimidin-4-yl)amino)-5-(6-methoxypyridazin-3-yl)pyridin-2-yl)acetamide trifluoroacetate

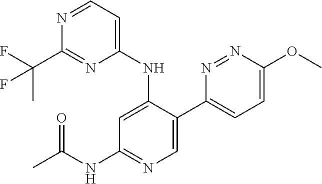 [SMILES: COc1ccc(-c2cnc(NC(C)=O)cc2Nc2ccnc(C(C)(F)F)n2)nn1]

A mixture of N-(4-amino-5-(6-methoxypyridazin-3-yl)pyridin-2-yl)acetamide (Preparation 434, 50 mg, 0.19 mmol), 4-chloro-2-(1,1-difluoroethyl)pyrimidine (Preparation 148, 50 mg, 0.29 mmol), K₃PO₄ (82 mg, 0.39 mmol), and [XantPhos Pd(allyl)]C1 (7.3 mg, 9.6 mmol) in 2-MeTHF (2 mL) was stirred at 90° C. overnight. The reaction was cooled to rt, diluted with EtOAc (5 mL), filtered through a 0.2 um syringe filter, and rinsed with EtOAc (5 mL). The filtrate was concentrated to dryness and the crude mixture was purified via prep-HPLC (Method V, Gradient 5-95%) to give N-(4-((2-(1,1-difluoroethyl)pyrimidin-4-yl)amino)-5-(6-methoxypyridazin-3-yl)pyridin-2-yl)acetamide trifluoroacetate (6.3 mg, 8% yield). LCMS m/z=402.1 [M+H]⁺. ¹H NMR (600 MHz, DMSO-d₆) δ (ppm)=11.48 (s, 1H), 10.64 (s, 1H), 9.09-8.96 (m, 1H), 8.68 (s, 1H), 8.58-8.49 (m, 1H), 8.19 (d, J=9.5 Hz, 1H), 7.39 (d, J=9.5 Hz, 1H), 7.10-7.07 (m, 1H), 4.10 (s, 3H), 2.14 (s, 3H), 2.04 (s, 3H).

### Example 215

### N-(4-((6-(2-fluoropropan-2-yl)pyridin-2-yl)amino)-5-(5-fluoropyrimidin-2-yl)pyridin-2-yl)acetamide

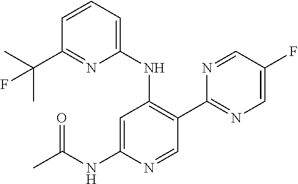 [SMILES: CC(=O)Nc1cc(Nc2cccc(C(C)(C)F)n2)c(-c2ncc(F)cn2)cn1]

To a solution of 2-bromo-6-(2-fluoropropan-2-yl)pyridine (Preparation 130, 100 mg, 0.459 mmol) in dioxane (10 mL) was added N-(4-amino-5-(5-fluoropyrimidin-2-yl)pyridin-2-yl)acetamide hydrochloride (Preparation 460, 113.4 mg, 0.459 mmol), Cs₂CO₃ (298.8 mg, 0.917 mmol) and Brettphos Pd G3 (41.6 mg, 45.9 umol). The resulting mixture was stirred at 100° C. for 2 h under N₂. The mixture was concentrated and purified by Prep-HPLC (Method B, 40-70%) to give N-(4-((6-(2-fluoropropan-2-yl)pyridin-2-yl)amino)-5-(5-fluoropyrimidin-2-yl)pyridin-2-yl)acetamide (50.8 mg, 28.8% yield) as a white solid. LCMS m/z=385.1 [M+H]⁺. ¹H NMR (400 MHz, CDCl₃) δ ppm: 9.33 (s, 2H), 8.71 (s, 2H), 8.36 (br s, 1H), 7.67 (t, J=7.6 Hz, 1H), 7.18-7.23 (m, 1H), 6.88-6.94 (m, 1H), 2.23 (s, 3H), 1.86 (s, 3H), 1.80 (s, 3H).

### Example 216

### N-(4-((2-(2-fluoropropan-2-yl)pyridin-4-yl)amino)-5-(5-fluoropyrimidin-2-yl)pyridin-2-yl)acetamide H

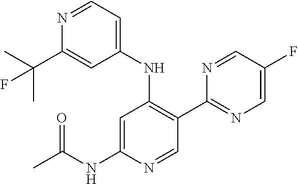 [SMILES: CC(=O)Nc1cc(Nc2ccnc(C(C)(C)F)c2)c(-c2ncc(F)cn2)cn1]

N-(4-((2-(2-Fluoropropan-2-yl)pyridin-4-yl)amino)-5-(5-fluoropyrimidin-2-yl)pyridin-2-yl)acetamide was obtained as a white solid, 10.8 mg, 28% yield, from 4-bromo-2-(2-fluoropropan-2-yl)pyridine and N-(4-amino-5-(5-fluoropyrimidin-2-yl)pyridin-2-yl)acetamide hydrochloride (Preparation 460), following a similar procedure to that described in Example 215. LCMS m/z=385.1 [M+H]⁺. 1H NMR (400 MHz, CDCl₃) δ ppm 11.21 (s, 1H), 9.08-9.35 (m, 1H), 8.59 (s, 2H), 8.22-8.43 (m, 3H), 7.17 (dd, J=5.6 Hz, 2.4 Hz, 1H), 7.14 (s, 1H), 2.10 (s, 3H), 1.66 (s, 3H), 1.60 (s, 3H)

### Example 217

### N-(4-((2-(1,1-difluoroethyl)pyridin-4-yl)amino)-5-(6-methoxypyrimidin-4-yl)pyridin-2-yl)acetamide

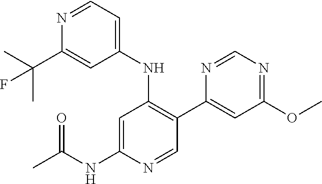 [SMILES: COc1cc(-c2cnc(NC(C)=O)cc2Nc2ccnc(C(C)(C)F)c2)ncn1]

N-(4-((2-(1,1-difluoroethyl)pyridin-4-yl)amino)-5-(6-methoxypyrimidin-4-yl)pyridin-2-yl)acetamide was obtained as a white solid, 65.7 mg, 64%, from N-(4-amino-5-(6-methoxypyrimidin-4-yl)pyridin-2-yl)acetamide hydrochloride (Preparation 458) and 4-chloro-2-(1,1-difluoroethyl)pyridine, following a similar procedure to that described in Example 215. LCMS m/z=401.1 [M+H]⁺ ¹H NMR (400 MHz, CDCl₃) δ ppm 11.71 (s, 1H), 8.87 (d, J=0.8 Hz, 1H), 8.53-8.56 (m, 2H), 8.43 (s, 1H), 8.12 (br s, 1H), 7.48 (d, J=2.0 Hz, 1H), 7.36 (dd, J=5.6 Hz, 2.0 Hz, 1H), 7.12 (s, 1H), 4.07 (s, 3H), 2.24 (s, 3H), 2.05 (t, J=18.8 Hz, 3H)

### Example 218

### N-(4-((2-(1,1-difluoroethyl)-6-methylpyrimidin-4-yl)amino)-5-(1-methyl-1H-pyrazol-3-yl)pyridin-2-yl)acetamide

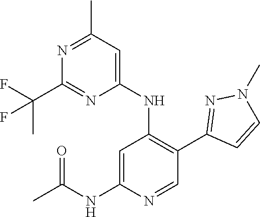 [SMILES: CC(=O)Nc1cc(Nc2cc(C)nc(C(C)(F)F)n2)c(-c2ccn(C)n2)cn1]

To a stirring solution of N-(4-amino-5-(1-methyl-1H-pyrazol-3-yl)pyridin-2-yl)acetamide (Preparation 481, 35 mg, 0.151 mmol) and 4-chloro-2-(1,1-difluoroethyl)-6-methylpyrimidine (Preparation 92, 37.9 mg, 0.197 mmol) in dioxane (1 mL) was added Cs₂CO₃ (98.6 mg, 0.303 mmol) and the resulting mixture was degassed using N₂ for 15 mins. BrettPhos Pd G3 (27.44 mg, 30.3 umol) and BrettPhos (32.50 mg, 60.5 umol) were added and the reaction mixture was heated under reflux at 100° C. for 16 h. The reaction was filtered through Celite® and the filtrate concentrated in vacuo. The crude was purified by HPLC (Method V, Gradient 5-95%) to give N-(4-((2-(1,1-difluoroethyl)-6-methylpyrimidin-4-yl)amino)-5-(1-methyl-1H-pyrazol-3-yl)pyridin-2-yl)acetamide as a white solid, 9.5 mg, 16.2%. LCMS m/z=388.3 [M+H]⁺; ¹H NMR (600 MHz, DMSO-d₆) δ 11.55 (s, 1H), 10.55 (s, 1H), 9.32 (s, 1H), 8.70 (s, 1H), 7.94 (d, J=2.4 Hz, 1H), 7.11 (s, 1H), 6.96 (d, J=2.4 Hz, 1H), 4.07 (s, 3H), 2.23-2.11 (m, 6H).

### Example 219

### N-(4-((2-(1,1-difluoroethyl)-6-methylpyrimidin-4-yl)amino)-5-(6-methylpyridazin-3-yl)pyridin-2-yl)acetamide

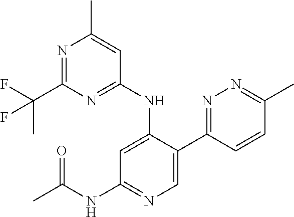 [SMILES: CC(=O)Nc1cc(Nc2cc(C)nc(C(C)(F)F)n2)c(-c2ccc(C)nn2)cn1]

A mixture of 4-chloro-2-(1,1-difluoroethyl)-6-methylpyrimidine (Preparation 92) (115 mg, 0.597 mmol), N-(4-amino-5-(6-methylpyridazin-3-yl)pyridin-2-yl)acetamide hydrochloride (Preparation 471, 102 mg, 0.365 mmol), Cs₂CO₃ (333 mg, 1.02 mmol), BINAP (80 mg, 0.129 mmol), DIPEA (1.4 mL, 8.04 mmol) and KOAc (11 mg, 49 umol) in dioxane (2 mL) was degassed then backfilled with N₂, heated to 95° C. and stirred for 8 h. The cooled mixture was purified directly by silica gel chromatography (10-85% 3:1 EtOAc. EtOH in heptane) The product was further purified by HPLC (Method U, Gradient 5-55%) to give a white solid as N-(4-((2-(1,1-difluoroethyl)-6-methylpyrimidin-4-yl)amino)-5-(6-methylpyridazin-3-yl)pyridin-2-yl)acetamide (8.5 mg, 5% yield). LCMS m/z=399.9 [M+H]+. 1H NMR (500 MHz, DMSO-d6) δ (ppm)=11.93 (s, 1H), 10.64 (s, 1H), 9.14 (s, 1H), 8.74 (s, 1H), 8.22 (d, J=8.9 Hz, 1H), 7.74 (d, J=9.2 Hz, 1H), 6.96 (s, 1H), 2.68 (s, 3H), 2.42 (s, 3H), 2.13 (s, 3H), 2.05 (br t, J=19.2 Hz, 3H).

### Example 220 and 221

### N-(4-((2-(1,1-difluoroethyl)-6-ethylpyrimidin-4-yl)amino)-5-(6-methoxypyridazin-3-yl)pyridin-2-yl)acetamide and N-(4-((2-(1,1-difluoroethyl)-6-ethylpyrimidin-4-yl)amino)-5-(6-oxo-1,6-dihydropyridazin-3-yl)pyridin-2-yl)acetamide

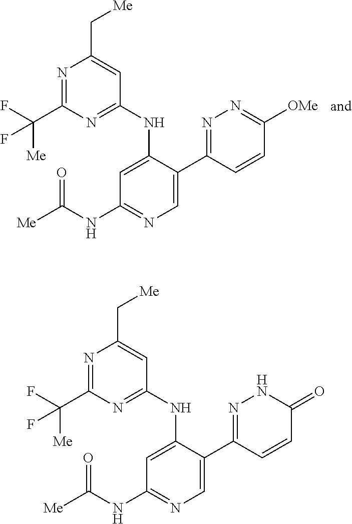 [SMILES: CCc1cc(Nc2cc(NC(C)=O)ncc2-c2ccc(OC)nn2)nc(C(C)(F)F)n1 ; "and" ; CCc1cc(Nc2cc(NC(C)=O)ncc2-c2ccc(=O)[nH]n2)nc(C(C)(F)F)n1]

To a solution of mixture of N-(4-amino-5-(6-methoxypyridazin-3-yl)pyridin-2-yl)acetamide hydrochloride and N-(4-amino-5-(6-oxo-1,6-dihydropyridazin-3-yl)pyridin-2-yl)acetamide hydrochloride (Preparation 450, 150 mg, 0.51 mmol) in dioxane (5 mL) was added Xantphos (58.7 mg, 0.10 mmol), 4-chloro-2-(1,1-difluoroethyl)-6-ethylpyrimidine (Preparation 94, 104.8 mg, 0.51 mmol), Cs₂CO₃ (331 mg, 1.01 mmol), Pd₂(dba)₃ (46.45 mg, 0.051 mmol) and the mixture stirred at 100° C. for 2 h under N₂. The reaction mixture was concentrated and the residue purified by prep-HPLC (Method R, Gradient 35-64%) to afford:

Peak 1, N-(4-((2-(1,1-difluoroethyl)-6-ethylpyrimidin-4-yl)amino)-5-(6-methoxypyridazin-3-yl)pyridin-2-yl)acetamide (white solid: 35.5 mg, 16%). LCMS m/z=430.1 [M+H]⁺; ¹H NMR (400 MHz, CDCl₃) δ ppm: 12.19 (s, 1H), 9.45 (s, 1H), 8.46 (s, 1H), 7.86-7.88 (m, 2H), 7.17 (d, J=9.2 Hz, 1H), 7.83 (s, 1H), 4.22 (s, 3H), 2.81 (q, J=7.2 Hz, 2H), 2.14-2.25 (m, 6H), 1.34 (t, J=7.2 Hz, 3H).

Peak 2, N-(4-((2-(1,1-difluoroethyl)-6-ethylpyrimidin-4-yl)amino)-5-(6-oxo-1,6-dihydropyridazin-3-yl)pyridin-2-yl)acetamide (white solid: 30.2 mg, 14%). LCMS m/z=416.1 [M+H]⁺; ¹H NMR (400 MHz, DMSO-d₆) δ ppm: 10.24 (s, 1H), 8.55 (s, 1H), 8.30-8.33 (m, 2H), 7.85 (br s, 1H), 7.60 (s, 1H), 7.22 (d, J=10.0 Hz, 1H), 2.94 (q, J=7.2 Hz, 2H), 2.00-2.10 (m, 6H), 1.30 (t, J=7.6 Hz, 3H).

### Example 222

### N-(5-(5-acetyl-4,5,6,7-tetrahydropyrazolo[1,5-a]pyrazin-2-yl)-4-((2-(1,1-difluoroethyl)-6-methylpyrimidin-4-yl)amino)pyridin-2-yl)acetamide trifluoroacetate

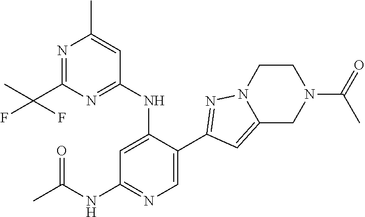 [SMILES: CC(=O)Nc1cc(Nc2cc(C)nc(C(C)(F)F)n2)c(-c2cc3n(n2)CCN(C(C)=O)C3)cn1]

A vial containing 1-(2-iodo-6,7-dihydropyrazolo[1,5-a]pyrazin-5(4H)-yl)ethan-1-one (Preparation 337, 111 mg, 0.382 mmol), N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 334 mg, 0.77 mmol), SPhos-Pd-G3 (39 mg, 50 mol) and K₃PO₄ (1.5 M aqueous solution, 0.8 mL, 1.2 mmol) in 2-MeTHF (2 mL) was degassed then backfilled with N₂ then heated at 75° C. for 3 h. The mixture was cooled to rt and filtered through Celite®, washing through with EtOAc. The filtrate was washed with brine, dried over Na₂SO₄ and concentrated in vacuo. The residue was purified by silica gel column chromatography (20-100% 3:1 EtOAc: EtOH in heptane). The product was further purified by HPLC (Method V: Gradient 5-40%) to give N-(5-(5-acetyl-4,5,6,7-tetrahydropyrazolo[1,5-a]pyrazin-2-yl)-4-((2-(1,1-difluoroethyl)-6-methylpyrimidin-4-yl)amino)pyridin-2-yl)acetamide (TFA Salt) (6 mg, 3% yield) as a white solid. LCMS m/z=471.2 [M+H]⁺. ¹H NMR (500 MHz, DCM-d₂) δ (ppm)=12.34 (br d, J=19.8 Hz, 1H), 12.09 (br d, J=11.0 Hz, 1H), 9.96-9.88 (m, 1H), 8.40 (d, J=8.2 Hz, 1H), 6.86 (s, 1H), 6.57 (d, J=3.7 Hz, 1H), 4.93 (s, 1H), 4.85 (s, 1H), 4.44-4.34 (m, 2H), 4.18 (br t, J=5.3 Hz, 1H), 4.03 (br t, J=5.0 Hz, 1H), 2.60 (s, 3H), 2.31 (s, 3H), 2.27 (d, J=15.3 Hz, 3H), 2.16 (t, J=18.9 Hz, 3H).

### Example 223

### N-(4-((2-(1,1-difluoroethyl)-6-methylpyrimidin-4-yl)amino)-5-(4-oxo-4,5,6,7-tetrahydropyrazolo[1,5-a]pyrazin-2-yl)pyridin-2-yl)acetamide

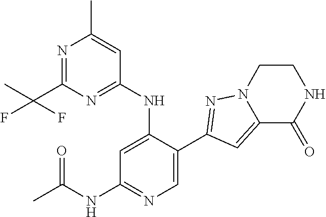 [SMILES: CC(=O)Nc1cc(Nc2cc(C)nc(C(C)(F)F)n2)c(-c2cc3n(n2)CCNC3=O)cn1]

N-(4-((2-(1,1-difluoroethyl)-6-methylpyrimidin-4-yl)amino)-5-(4-oxo-4,5,6,7-tetrahydropyrazolo[1,5-a]pyrazin-2-yl)pyridin-2-yl)acetamide was obtained, 3 mg, 2%, from 2-bromo-6,7-dihydropyrazolo[1,5-a]pyrazin-4(5H)-one (Preparation 344) and N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486) following a similar procedure to that described in Example 222. LCMS m/z=443.1 [M+H]⁺. ¹H NMR (500 MHz, DMSO-d₆) δ (ppm)=11.03 (s, 1H), 10.48 (s, 1H), 9.27 (s, 1H), 8.75 (s, 1H), 8.38 (br s, 1H), 7.40 (s, 1H), 7.08 (s, 1H), 4.51 (t, J=6.1 Hz, 2H), 3.73-3.67 (m, 2H), 2.45 (s, 3H), 2.15-2.07 (m, 6H).

### Example 224

### N-(4-((2-(1,1-difluoroethyl)-6-methylpyrimidin-4-yl)amino)-5-(7-hydroxy-5-methyl-4,5,6,7-tetrahydropyrazolo[1,5-a]pyrazin-2-yl)pyridin-2-yl)acetamide

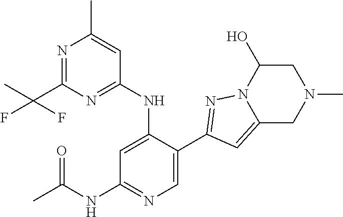 [SMILES: CC(=O)Nc1cc(Nc2cc(C)nc(C(C)(F)F)n2)c(-c2cc3n(n2)C(O)CN(C)C3)cn1]

A vial containing 2-bromo-7-fluoro-5-methyl-4,5,6,7-tetrahydropyrazolo[1,5-a]pyrazine (Preparation 350, 100 mg, 0.427 mmol), N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 317 mg, 0.732 mmol), PCy₃-Pd-G3 (64 mg, 88 mol), and KOAc (1.5 M, 1.50 mmol, 1 mL) in dioxane (4 mL) was degassed then backfilled with N₂ then heated at 90° C. for 2.5 h. The cooled mixture was purified directly by silica gel column chromatography (20-100% EtOAc with 2% dimethylethylamine in heptane then 100% 3:1 EtOAc: EtOH.) The product was further purified by HPLC (Method U, Gradient 5-55%) toto give N-(4-((2-(1,1-difluoroethyl)-6-methylpyrimidin-4-yl)amino)-5-(7-hydroxy-5-methyl-4,5,6,7-tetrahydropyrazolo[1,5-a]pyrazin-2-yl)pyridin-2-yl)acetamide (3 mg, 2% yield) as a light-yellow solid. LCMS m/z=459.2 [M+H]⁺. ¹H NMR (500 MHz, DMSO-d₆) δ (ppm)=11.67 (s, 1H), 10.43 (s, 1H), 9.40 (s, 1H), 8.64 (s, 1H), 7.24 (d, J=6.4 Hz, 1H), 7.12 (s, 1H), 6.72 (s, 1H), 5.76-5.70 (m, 1H), 3.72-3.57 (m, 2H), 3.04 (br dd, J=4.0, 12.2 Hz, 1H), 2.74 (br dd, J=4.9, 12.5 Hz, 1H), 2.46 (s, 3H), 2.41 (s, 3H), 2.19-2.08 (m, 6H).

### Example 225

### N-(4-((2-(1,1-difluoroethyl)-6-methylpyrimidin-4-yl)amino)-5-(5-(dimethylamino)pyrazin-2-yl)pyridin-2-yl)acetamide

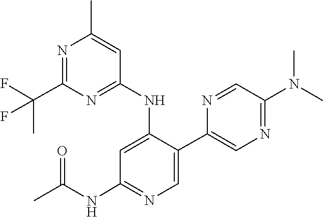 [SMILES: CC(=O)Nc1cc(Nc2cc(C)nc(C(C)(F)F)n2)c(-c2cnc(N(C)C)cn2)cn1]

A mixture of N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 25 mg, 0.058 mmol), Pd(OAc)₂ (1.3 mg, 5.8 mmol), PCy₃ (3.2 mg, 12 mmol), 5-bromo-N,N-dimethyl-pyrazin-2-amine (23 mg, 0.115 mmol), K₂CO₃ (16 mg, 0.115 mmol), 2-MeTHF (2.0 mL) and water (1.0 mL) was stirred for 2 h at 90° C. The reaction was cooled to rt, diluted with water (3 mL) and extracted with 3:1 EtOAc:EtOH (3×3 mL). The combined organic layers were concentrated to dryness and purified via prep-HPLC (Method U, Gradient 5-95%) to give N-(4-((2-(1,1-difluoroethyl)-6-methylpyrimidin-4-yl)amino)-5-(5-(dimethylamino)pyrazin-2-yl)pyridin-2-yl)acetamide (2.4 mg, 10% yield). LCMS m/z=429.2 [M+H]⁺.

### Example 226

### N-(4-((2-(1,1-difluoroethyl)-6-methylpyrimidin-4-yl)amino)-5-(5-methyl-4,5,6,7-tetrahydropyrazolo[1,5-a]pyrazin-2-yl)pyridin-2-yl)acetamide

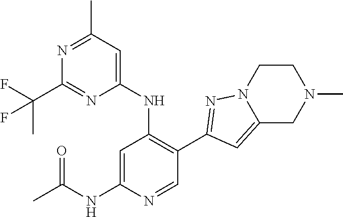 [SMILES: CC(=O)Nc1cc(Nc2cc(C)nc(C(C)(F)F)n2)c(-c2cc3n(n2)CCN(C)C3)cn1]

To a solution of (6-acetamido-4-((2-(1,1-difluoroethyl)-6-methylpyrimidin-4-yl)amino)pyridin-3-yl)boronic acid (Preparation 488, 100 mg, 0.285 mmol) in dioxane (1 mL) and water (0.2 mL) was added 2-bromo-5-methyl-4,5,6,7-tetrahydropyrazolo[1,5-a]pyrazine (Preparation 331, 123.1 mg, 0.57 mmol), Pd(dppf)Cl₂·DCM (23.26 mg, 28.5 umol), K₂CO₃ (118.09 mg, 0.854 mmol) and the mixture was stirred at 90° C. for 2 h under N₂. The mixture was concentrated and purified by Prep-HPLC (Method R, Gradient 30-60%) to give N-(4-((2-(1,1-difluoroethyl)-6-methylpyrimidin-4-yl)amino)-5-(5-methyl-4,5,6,7-tetrahydropyrazolo[1,5-a]pyrazin-2-yl)pyridin-2-yl)acetamide (65 mg, 51.6% yield) as a white solid. LCMS m/z=443.3 [M+H]⁺. 1H NMR (400 MHz, CDCl₃) δ ppm 11.37 (s, 1H), 9.36 (s, 1H), 8.44 (s, 1H), 7.93 (s, 1H), 6.73 (s, 1H), 6.37 (s, 1H), 4.32 (t, J=5.6 Hz, 2H), 3.71 (s, 2H), 2.97 (t, J=5.6 Hz, 2H), 2.48-2.62 (m, 6H), 2.10-2.28 (m, 6H).

### Examples 227 to 254

The compounds in the following table were prepared from (6-acetamido-4-((2-(1,1-difluoroethyl)-6-methylpyrimidin-4-yl)amino)pyridin-3-yl)boronic acid (Preparation 488) and the appropriate halohetereocycle, following a similar procedure described in Example 226.

| Example No. | Name, Structure, Starting Material (SM), Data |
|---|---|
| 227 | N-(4-((2-(1,1-difluoroethyl)-6-methylpyrimidin-4-yl)amino)-5-(2- |
| | methyl-2H-1,2,3-triazol-4-yl)pyridin-2-yl)acetamide |
| | 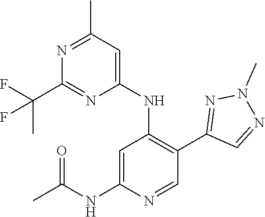 [SMILES: CC(=O)Nc1cc(Nc2cc(C)nc(C(C)(F)F)n2)c(-c2cnn(C)n2)cn1] |
| | SM: 4-bromo-2-methyl-2H-1,2,3-triazole |
| | Prep-HPLC (Method H, 25% to 45%). 15 mg, 13.6% yield as a white |
| | solid. LCMS m/z = 389.1 [M + H]⁺ 1H NMR: (400 MHz, DMSO-d₆) δ |
| | ppm 10.67 (s, 1H), 10.24 (s, 1H), 9.11-9.15 (m, 1H), 8.68 (d, J = 1.2 Hz, |
| | 1H), 8.32 (d, J = 4.0 Hz, 1H), 7.09 (d, J = 3.2 Hz, 1H), 4.30 (s, 3H), 2.45 |
| | (s, 3H), 2.02-2.14 (m, 6H) |
| 228 | N-(4-((2-(1,1-difluoroethyl)-6-methylpyrimidin-4-yl)amino)-5-(1-(2- |
| | hydroxy-2-methylpropyl)-1H-pyrazol-3-yl)pyridin-2-yl)acetamide |
| | 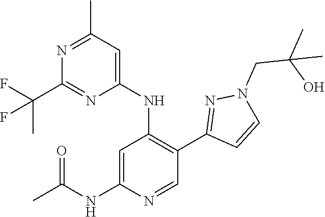 [SMILES: CC(=O)Nc1cc(Nc2cc(C)nc(C(C)(F)F)n2)c(-c2ccn(CC(C)(C)O)n2)cn1] |
| | SM: 1-(3-iodo-1H-pyrazol-1-yl)-2-methylpropan-2-ol (Example 82, step |
| | 2, US11590111B2) |
| | Prep-HPLC (Method M, Gradient 19-39%). 18 mg, 14.6% yield as a |
| | yellow solid. ICMS m/z = 433.1 [M + H]⁺ ¹H NMR (400 MHz, CDCl₃) δ: |
| | ppm 11.37 (s, 1H), 9.38 (s, 1H), 9.03 (s, 1H), 8.26 (s, 1H), 8.08 (s, 1H), |
| | 6.81 (s, 1H), 4.43 (t, J = 4.8 Hz, 2H), 3.81 (t, J = 4.8 Hz, 2H), 3.38 (s, 3H), |
| | 2.55 (s, 3H), 2.15-2.24 (m, 6H). |
| 229 | N-(4-((2-(1,1-difluoroethyl)-6-methylpyrimidin-4-yl)amino)-5-(1-(2- |
| | ethoxyethyl)-1H-pyrazol-3-yl)pyridin-2-yl)acetamide |
| | 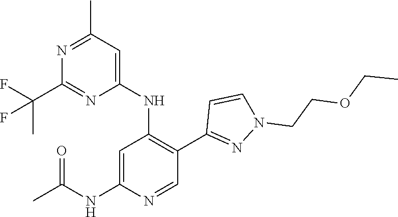 [SMILES: CCOCCn1ccc(-c2cnc(NC(C)=O)cc2Nc2cc(C)nc(C(C)(F)F)n2)n1] |
| | SM: 3-bromo-1-(2-ethoxyethyl)-1H-pyrazole (Preparation 289) |
| | Prep-HPLC (Method G, 33-63%). 15 mg, 14.8% yield as a white solid. |
| | LCMS m/z = 446.2 [M + H]⁺ ¹H NMR (400 MHz, CDCl₃) δ ppm: 11.36 (s, |
| | 1H), 9.29 (s, 1H), 8.49 (s, 1H), 7.86 (br s, 1H), 7.59 (d, J = 2.8 Hz, 1H), 6.77 |
| | (s, 1H), 6.64 (d, J = 2.4 Hz, 1H), 4.40 (t, J = 5.2 Hz, 2H), 3.88 (t, J = 5.2 Hz, |
| | 2H), 3.51 (q, J = 7.2 Hz, 2H), 2.55 (s, 3H), 2.13-2.24 (m, 6H), 1.18 (t, J = 6.8 |
| | Hz, 3H). |
| 230 | N-(4-((2-(1,1-difluoroethyl)-6-methylpyrimidin-4-yl)amino)-5-(1-(2- |
| | methoxy-2-methylpropyl)-1H-pyrazol-3-yl)pyridin-2-yl)acetamide |
| | 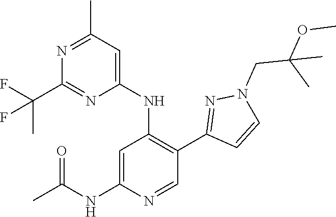 [SMILES: COC(C)(C)Cn1ccc(-c2cnc(NC(C)=O)cc2Nc2cc(C)nc(C(C)(F)F)n2)n1] |
| | SM: 3-iodo-1-(2-methoxy-2-methylpropyl)-1H-pyrazole (Preparation 297) |
| | prep-HPLC (Method R, Gradient 10-40%). 42.8 mg, 32.7% yield as a |
| | white solid. LCMS m/z = 460.2 [M + H]⁺ ¹H NMR (400 MHz, DMSO-d₆) |
| | δ ppm: 11.59 (s, 1H), 10.46 (s, 1H), 9.29 (s, 1H), 8.68 (s, 1H), 7.83 (d, |
| | J = 2.4 Hz, 1H), 7.03 (s, 1H), 6.95 (d, J = 2.4 Hz, 1H), 4.34 (s, 2H), 3.21 (s, |
| | 3H), 2.45 (s, 3H), 2.07-2.17 (m, 6H), 1.15 (s, 6H). |
| 231 | N-(4-((2-(1,1-difluoroethyl)-6-methylpyrimidin-4-yl)amino)-5-(5- |
| | (methoxymethyl)thiazol-2-yl)pyridin-2-yl)acetamide |
| | 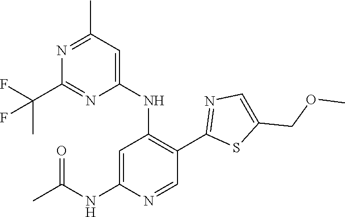 [SMILES: COCc1cnc(-c2cnc(NC(C)=O)cc2Nc2cc(C)nc(C(C)(F)F)n2)s1] |
| | SM: 2-bromo-5-(methoxymethyl)thiazole (Preparation 293) |
| | prep-HPLC (Method R, Gradient 36-65%). 36.1 mg, 5.83% yield as a |
| | white solid. LCMS m/z = 435.1 [M + H]⁺. ¹H NMR (400 MHz, CDCl₃) δ |
| | ppm: 12.27 (s, 1H), 9.51 (s, 1H), 8.60 (s, 1H), 7.89 (s, 1H), 7.74 (s, 1H), |
| | 6.78 (s, 1H), 4.70 (s, 2H), 3.44 (s, 3H), 2.56 (s, 3H), 2.15-2.25 (m, 6H). |
| 232 | N-(4-((2-(1,1-difluoroethyl)-6-methylpyrimidin-4-yl)amino)-5-(2- |
| | (methoxymethyl)thiazol-5-yl)pyridin-2-yl)acetamide |
| | 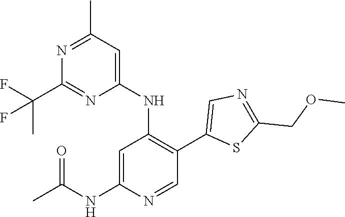 [SMILES: COCc1ncc(-c2cnc(NC(C)=O)cc2Nc2cc(C)nc(C(C)(F)F)n2)s1] |
| | SM: 5-bromo-2-(methoxymethyl)thiazole (Preparation 294) |
| | prep-HPLC (Method R, Gradient 33-63%). 36.6 mg, 29.6% yield as a |
| | white solid. LCMS m/z = 435.1 [M + H]⁺. ¹H NMR (400 MHz, CDCl₃) δ |
| | ppm: 11.24 (s, 1H), 9.23 (s, 1H), 8.49 (s, 1H), 7.92 (s, 1H), 7.56 (s, 1H), |
| | 6.74 (s, 1H), 4.88 (s, 2H), 3.60 (s, 3H), 2.54 (s, 3H), 2.24 (s, 3H), 2.16 (t, |
| | J = 18.8 Hz, 3H). |
| 233 | N-(4-((2-(1,1-difluoroethyl)-6-methylpyrimidin-4-yl)amino)-5-(4- |
| | (methoxymethyl)thiazol-2-yl)pyridin-2-yl)acetamide |
| | 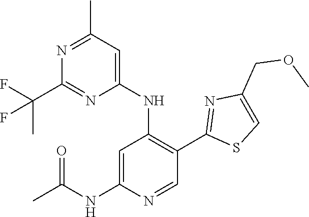 [SMILES: COCc1csc(-c2cnc(NC(C)=O)cc2Nc2cc(C)nc(C(C)(F)F)n2)n1] |
| | SM: 2-bromo-4-(methoxymethyl)thiazole (Preparation 295) |
| | Prep-HPLC (Method K, Gradient 39-69%). 50 mg, 40.4% yield as a |
| | white solid. LCMS m/z = 435.1 [M + H]⁺ 1H NMR (400 MHz, CDCl₃) δ |
| | ppm 12.38 (s, 1H), 9.52 (s, 1H), 8.62 (s, 1H), 7.91 (s, 1H), 7.22 (s, 1H), |
| | 6.72 (s, 1H), 4.69 (s, 2H), 3.54 (s, 3H), 2.56 (s, 3H), 2.14-2.28 (m, 6H). |
| 234 | N-(4-((2-(1,1-difluoroethyl)-6-methylpyrimidin-4-yl)amino)-5-(2- |
| | morpholinothiazol-4-yl)pyridin-2-yl)acetamide |
| | 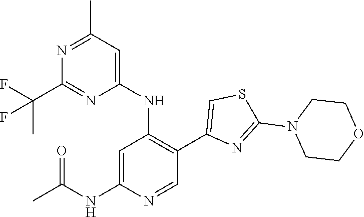 [SMILES: CC(=O)Nc1cc(Nc2cc(C)nc(C(C)(F)F)n2)c(-c2csc(N3CCOCC3)n2)cn1] |
| | SM: 4-(4-bromothiazol-2-yl)morpholine |
| | Prep-HPLC (Method B, 40-70%. 15.85 mg, 12.6% yield as a pink solid. |
| | LCMS m/z = 476.2 [M + H]⁺ 1H NMR (400 MHz, CDCl₃) δ ppm: 11.67 (s, |
| | 1H), 8.79 (s, 1H), 8.63 (s, 1H), 8.43 (s, 1H), 6.96 (s, 1H), 6.80 (s, 1H), 3.88 |
| | (t, J = 4.4 Hz, 4H), 3.61 (t, J = 4.8 Hz, 4H), 2.53 (s, 3H), 2.22 (s, 3H), 2.03 (t, |
| | J = 18.8 Hz, 3H). |
| 235 | N-(4-((2-(1,1-difluoroethyl)-6-methylpyrimidin-4-yl)amino)-5-(1,2,4- |
| | thiadiazol-5-yl)pyridin-2-yl)acetamide |
| | 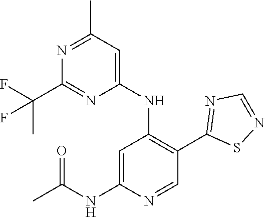 [SMILES: CC(=O)Nc1cc(Nc2cc(C)nc(C(C)(F)F)n2)c(-c2ncns2)cn1] |
| | SM: 5-bromo-1,2,4-thiadiazole |
| | prep-HPLC (Method S, Gradient 10-40%) 12.2 mg, 10.9% yield as a white |
| | solid. LCMS m/z = 392.1 [M + H]⁺ ¹H NMR (400 MHz, DMSO-d₆) δ ppm: |
| | 11.21 (s, 1H), 10.87 (s, 1H), 9.13 (s, 1H), 9.02 (s, 1H), 8.95 (s, 1H), 7.05 |
| | (s, 1H), 2.45 (s, 3H), 2.14 (s, 3H), 2.04 (t, J = 19.0 Hz, 3H). |
| 236 | N-(4-((2-(1,1-difluoroethyl)-6-methylpyrimidin-4-yl)amino)-5-(5- |
| | (dimethylamino)-1,3,4-thiadiazol-2-yl)pyridin-2-yl)acetamide |
| | 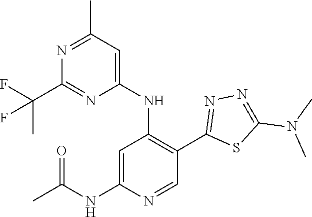 [SMILES: CC(=O)Nc1cc(Nc2cc(C)nc(C(C)(F)F)n2)c(-c2nnc(N(C)C)s2)cn1] |
| | SM: 5-bromo-N,N-dimethyl-1,3,4-thiadiazol-2-amine (Preparation 301) |
| | Prep-HPLC (Method D, 25-55%). 7.10 mg, 4.0% yield as a dark green |
| | solid. LCMS m/z = 435.1 [M + H]⁺ ¹H NMR (400 MHz, CDCl₃) δ ppm: |
| | 12.06 (s, 1H), 9.78 (s, 1H), 8.95-9.25 (m, 1H), 8.24 (s, 1H), 6.89 (s, 1H), |
| | 3.28 (s, 6H), 2.56 (s, 3H), 2.29 (s, 3H), 2.21 (t, J = 18.8 Hz, 1H). |
| 237 | N-(4-((2-(1,1-difluoroethyl)-6-methylpyrimidin-4-yl)amino)-5-(5- |
| | methyl-4,5,6,7-tetrahydrothiazolo[4,5-c]pyridin-2-yl)pyridin-2- |
| | yl)acetamide |
| | 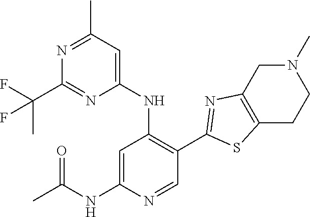 [SMILES: CC(=O)Nc1cc(Nc2cc(C)nc(C(C)(F)F)n2)c(-c2nc3c(s2)CCN(C)C3)cn1] |
| | SM: 2-bromo-5-methyl-4,5,6,7-tetrahydrothiazolo[4,5-c]pyridine |
| | Prep-HPLC (Method C, 50% to 75%). 20 mg, 15.3% yield as a yellow |
| | solid. LCMS m/z = 460.2 [M + H]⁺ ¹H NMR (400 MHz, CDCl₃) δ: ppm |
| | 12.13 (br s, 1H), 9.50 (s, 1H), 8.55 (s, 1H), 7.90 (s, 1H), 6.67 (s, 1H), 3.89 |
| | (br s, 2H), 2.99-3.07 (m, 4H), 2.69 (s, 3H), 2.57 (s, 3H), 2.15-2.25 (m, 6H). |
| 238 | N-(4-((2-(1,1-difluoroethyl)-6-methylpyrimidin-4-yl)amino)-5-(5- |
| | methyl-4,5,6,7-tetrahydrothiazolo[5,4-c]pyridin-2-yl)pyridin-2-yl)acetamide |
| | 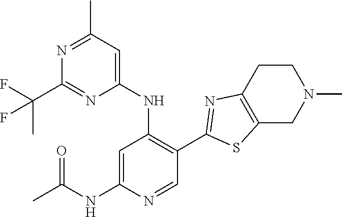 [SMILES: CC(=O)Nc1cc(Nc2cc(C)nc(C(C)(F)F)n2)c(-c2nc3c(s2)CN(C)CC3)cn1] |
| | SM: 2-bromo-5-methyl-4,5,6,7-tetrahydrothiazolo[5,4-c]pyridine |
| | prep-HPLC (Method R, Gradient 35-65%). 45 mg, 34.4% yield as a |
| | white solid. LCMS m/z = 460.2 [M + H]⁺ 1H NMR (400 MHz, CDCl₃) δ |
| | ppm 12.10 (s, 1H), 9.43 (s, 1H), 8.47 (s, 1H), 7.89 (s, 1H), 6.61 (s, 1H), |
| | 3.92 (s, 2H), 3.07-3.13 (m, 4H), 2.66 (s, 3H), 2.49 (s, 3H), 2.08-2.18 (m, 6H) |
| 239 | N-(4-((2-(1,1-difluoroethyl)-6-methylpyrimidin-4-yl)amino)-5-(7- |
| | methyl-5,6,7,8-tetrahydroimidazo[1,2-a]pyrazin-2-yl)pyridin-2-yl)acetamide |
| | 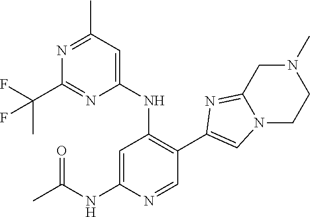 [SMILES: CC(=O)Nc1cc(Nc2cc(C)nc(C(C)(F)F)n2)c(-c2cn3c(n2)CN(C)CC3)cn1] |
| | SM: 2-bromo-7-methyl-5,6,7,8-tetrahydroimidazo[1,2-a]pyrazine |
| | (Preparation 332) |
| | Prep-HPLC (Method K, Gradient 27-57%). 30 mg, 23.8% yield as a |
| | white solid. LCMS m/z = 443.2 [M + H]⁺ 1H NMR (400 MHz, CDCl₃) δ |
| | ppm 12.11 (s, 1H), 9.37 (s, 1H), 8.33 (s, 1H), 7.88 (s, 1H), 7.17 (s, 1H), |
| | 6.68 (s, 1H), 4.10 (t, J = 5.2 Hz, 2H), 3.78 (s, 2H), 2.90 (t, J = 5.2 Hz, 2H), |
| | 2.56 (s, 3H), 2.52 (s, 3H), 2.15-2.25 (m, 6H) |
| 240 | N-(4-((2-(1,1-difluoroethyl)-6-methylpyrimidin-4-yl)amino)-5-(4- |
| | methyl-4,5,6,7-tetrahydropyrazolo[1,5-a]pyrimidin-2-yl)pyridin-2- |
| | yl)acetamide |
| | 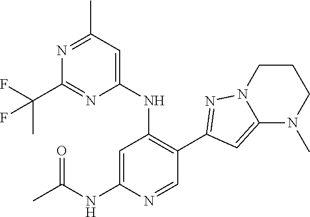 [SMILES: CC(=O)Nc1cc(Nc2cc(C)nc(C(C)(F)F)n2)c(-c2cc3n(n2)CCCN3C)cn1] |
| | SM: 2-bromo-4-methyl-4,5,6,7-tetrahydropyrazolo[1,5-a]pyrimidine |
| | (Preparation 334) |
| | prep-HPLC (Method R, Gradient 33-63%). 22 mg, 17.5% yield as a |
| | white solid. LCMS m/z = 443.1 [M + H]⁺ 1H NMR (400 MHz, CDCl₃) δ |
| | ppm: 11.60 (s, 1H), 9.36 (s, 1H), 8.41 (s, 1H), 8.25 (s, 1H), 6.69 (s, 1H), |
| | 5.69 (s, 1H), 4.15-4.19 (m, 2H), 3.16-3.19 (m, 2H), 2.91 (s, 3H), 2.52 (s, |
| | 3H), 2.14-2.29 (m, 8H). |
| 241 | N-(5-(6-(2-cyanopropan-2-yl)pyrimidin-4-yl)-4-((2-(1,1-difluoroethyl)-6- |
| | methylpyrimidin-4-yl)amino)pyridin-2-yl)acetamide |
| | 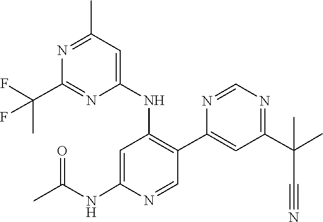 [SMILES: CC(=O)Nc1cc(Nc2cc(C)nc(C(C)(F)F)n2)c(-c2cc(C(C)(C)C#N)ncn2)cn1] |
| | SM: 2-(6-chloropyrimidin-4-yl)-2-methylpropanenitrile (Example 7, |
| | EP3036232B1) |
| | Prep-HPLC (Method C, 34% to 64%). 10 mg, 7.5% yield as a yellow solid. |
| | LCMS m/z = 453.3 [M + H]⁺ 1H NMR (400 MHz, CDCl₃) δ: ppm 12.56 (s, |
| | 1H), 9.49 (s, 1H), 9.30 (s, 1H), 8.76 (s, 1H), 8.70 (br s, 1H), 8.07 (s, 1H), |
| | 6.84 (s, 1H), 2.58 (s, 3H), 2.29 (s, 3H), 2.17 (t, J = 18.8 Hz, 3H), 1.83 (s, 6H). |
| 242 | N-(4-((2-(1,1-difluoroethyl)-6-methylpyrimidin-4-yl)amino)-5-(6- |
| | isopropoxypyrimidin-4-yl)pyridin-2-yl)acetamide |
| | 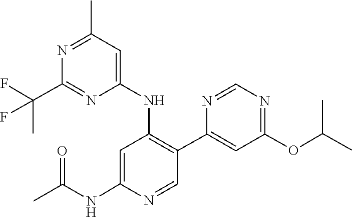 [SMILES: CC(=O)Nc1cc(Nc2cc(C)nc(C(C)(F)F)n2)c(-c2cc(OC(C)C)ncn2)cn1] |
| | SM: 4-chloro-6-isopropoxypyrimidine |
| | Prep-HPLC (Method R, Gradient 45-75%) . 40 mg, 31% yield as a white |
| | solid. LCMS m/z = 444.1 [M + H]⁺ 1H NMR (400 MHz, CDCl₃) δ ppm |
| | 12.50 (s, 1H), 9.35 (s, 1H), 8.84 (s, 1H), 8.55 (s, 1H), 7.88 (s, 1H), 7.05 |
| | (s, 1H), 6.76 (s, 1H), 5.34-5.59 (m, 1H), 2.55 (s, 3H), 2.12-2.27 (m, 6H), |
| | 1.41 (d, J = 6.4 Hz, 6H). |
| 243 | N-(4-((2-(1,1-difluoroethyl)-6-methylpyrimidin-4-yl)amino)-5-(6- |
| | ethoxypyrimidin-4-yl)pyridin-2-yl)acetamide |
| | 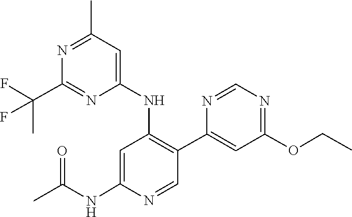 [SMILES: CCOc1cc(-c2cnc(NC(C)=O)cc2Nc2cc(C)nc(C(C)(F)F)n2)ncn1] |
| | SM: 4-chloro-6-ethoxypyrimidine |
| | Prep-HPLC (Method D, 35-65%). 13.7 mg, 11.2% yield as a white solid. |
| | LCMS m/z = 430.1 [M + H]⁺. 1H NMR (400 MHz, CDCl₃) δ ppm: 12.49 (s, |
| | 1H), 9.36 (s, 1H), 8.86 (s, 1H), 8.56 (s, 1H), 7.92 (s, 1H), 7.10 (s, 1H), 6.77 |
| | (s, 1H), 4.51 (q, J = 6.8 Hz, 2H), 2.55 (s, 3H), 2.25 (s, 3H), 2.18 (t, J = 18.8 |
| | Hz, 3H), 1.45 (t, J = 7.2 Hz, 3H). |
| 244 | N-(4-((2-(1,1-difluoroethyl)-6-methylpyrimidin-4-yl)amino)-5-(6- |
| | ethylpyrimidin-4-yl)pyridin-2-yl)acetamide |
| | 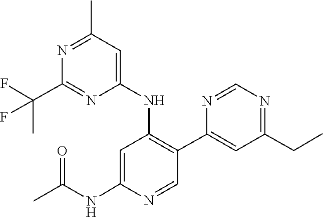 [SMILES: CCc1cc(-c2cnc(NC(C)=O)cc2Nc2cc(C)nc(C(C)(F)F)n2)ncn1] |
| | SM: 4-chloro-6-ethylpyrimidine |
| | prep-HPLC (Method R, Gradient 30-50%). 23.0 mg, 19.0% yield as a |
| | white solid. LCMS m/z = 414.2 [M + H]+ 1H NMR (500 MHz, CDCl₃) δ |
| | ppm 12.64 (s, 1H), 9.42 (s, 1H), 9.20 (d, J = 1.0 Hz, 1H), 8.66 (s, 1H), 7.94 |
| | (br s, 1H), 7.63 (s, 1H), 6.78 (s, 1H), 2.90 (q, J = 7.5 Hz, 2H), 2.56 (s, 3H), |
| | 2.26 (s, 3H), 2.18 (t, J = 18.5 Hz, 3H), 1.39 (t, J = 7.5 Hz, 3H). |
| 245 | N-(4-((2-(1,1-difluoroethyl)-6-methylpyrimidin-4-yl)amino)-5-(6- |
| | isopropylpyrimidin-4-yl)pyridin-2-yl)acetamide |
| | 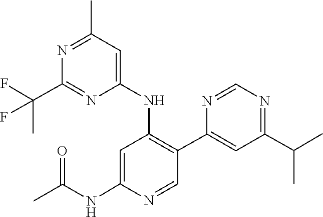 [SMILES: CC(=O)Nc1cc(Nc2cc(C)nc(C(C)(F)F)n2)c(-c2cc(C(C)C)ncn2)cn1] |
| | SM: 4-chloro-6-isopropylpyrimidine |
| | prep-HPLC (Method R, Gradient 25-65%). 36.6 mg, 29.9% yield as a |
| | white solid. LCMS m/z = 428.1 [M + H]⁺ ¹H NMR (400 MHz, DMSO-d₆) |
| | δ ppm 12.34 (br s, 1H), 10.67 (s, 1H), 9.22 (d, J = 3.2 Hz, 2H), 8.92 (s, 1H), |
| | 8.02 (s, 1H), 7.08 (s, 1H), 3.03-3.11 (m, 1H), 2.44 (s, 3H), 2.14 (s, 3H), |
| | 2.07 (t, J = 19.2 Hz, 3H), 1.28 (d, J = 6.8 Hz, 6H). |
| 246 | N-(4-((2-(1,1-difluoroethyl)-6-methylpyrimidin-4-yl)amino)-5-(6- |
| | (difluoromethyl)pyrimidin-4-yl)pyridin-2-yl)acetamide |
| | 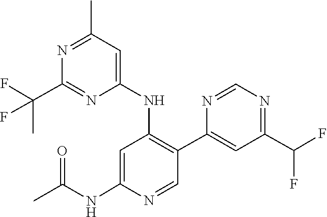 [SMILES: CC(=O)Nc1cc(Nc2cc(C)nc(C(C)(F)F)n2)c(-c2cc(C(F)F)ncn2)cn1] |
| | SM: 4-chloro-6-(difluoromethyl)pyrimidine |
| | Prep-HPLC (Method B, 36-66%). 12.5 mg, 10.1% yield as a white solid. |
| | LCMS m/z = 436.2 [M + H]⁺. 1H NMR (500 MHz, CDCl₃) δ ppm: 12.58 |
| | (s, 1H), 9.55 (s, 1H), 9.39 (s, 1H), 8.93 (s, 1H), 8.74-8.77 (m, 1H), 8.09 (s, |
| | 1H), 8.85 (s, 1H), 6.65 (t, J = 54.5 Hz, 1H), 2.59 (s, 3H), 2.30 (s, 3H), 2.17 |
| | (t, J = 19.0 Hz, 3H). |
| 247 | N-(4-((2-(1,1-difluoroethyl)-6-methylpyrimidin-4-yl)amino)-5-(6- |
| | (dimethylamino)pyrimidin-4-yl)pyridin-2-yl)acetamide |
| | 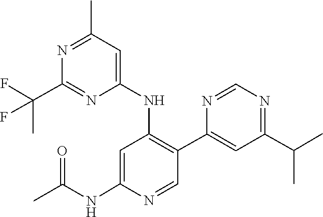 [SMILES: CC(=O)Nc1cc(Nc2cc(C)nc(C(C)(F)F)n2)c(-c2cc(C(C)C)ncn2)cn1] |
| | SM: 6-chloro-N,N-dimethylpyrimidin-4-amine |
| | prep-HPLC (Method X, Gradient 9-39%) and further purified by prep- |
| | HPLC (Method R, Gradient 32-62%). 10 mg, 8.20% yield as a pink solid. |
| | LCMS m/z = 429.2 [M + H]⁺ 1H NMR (400 MHz, DMSO-d₆) δ ppm: 12.87 |
| | (s, 1H), 10.54 (s, 1H), 9.25 (s, 1H), 8.85 (s, 1H), 8.66 (s, 1H), 7.13 (s, 1H), |
| | 7.05 (s, 1H), 3.16 (s, 6H), 2.43 (s, 3H), 2.05-2.43 (m, 6H). |
| 248 | N-(4-((2-(1,1-difluoroethyl)-6-methylpyrimidin-4-yl)amino)-5-(6-(4- |
| | methylpiperazin-1-yl)pyrimidin-4-yl)pyridin-2-yl)acetamide |
| | 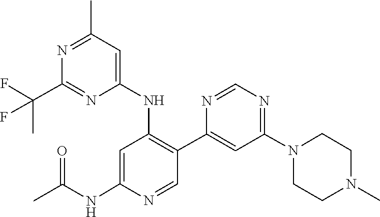 [SMILES: CC(=O)Nc1cc(Nc2cc(C)nc(C(C)(F)F)n2)c(-c2cc(N3CCN(C)CC3)ncn2)cn1] |
| | SM: 4-chloro-6-(4-methylpiperazin-1-yl)pyrimidine (Preparation 354) |
| | prep-HPLC (Method Y, Gradient 6-36%) then Prep-HPLC (Method G, 32- |
| | 62%) |
| | 14.44 mg, 36.1% yield as a yellow solid. LCMS m/z = 484.2 [M + H]⁺ |
| | 1H NMR (400 MHz, CDCl₃) δ ppm: 12.55 (s, 1H), 9.35 (br s, 1H), 8.68 |
| | (s, 1H), 8.50 (br s, 1H), 7.96 (br s, 1H), 6.83 (s, 1H), 6.71 (s, 1H), 3.86 |
| | (br s, 4H), 2.65 (br s, 4H), 2.53 (s, 3H), 2.45 (s, 3H), 2.24 (s, 3H), 2.17 (t, |
| | J = 18.8 Hz, 3H). |
| 249 | N-(4-((2-(1,1-difluoroethyl)-6-methylpyrimidin-4-yl)amino)-5-(6-(2- |
| | methoxyethoxy)pyrimidin-4-yl)pyridin-2-yl)acetamide |
| | 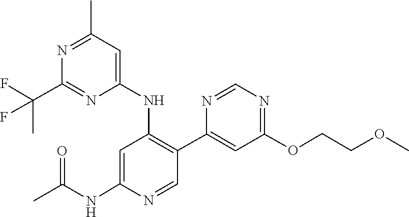 [SMILES: COCCOc1cc(-c2cnc(NC(C)=O)cc2Nc2cc(C)nc(C(C)(F)F)n2)ncn1] |
| | SM: 4-chloro-6-(2-methoxyethoxy)pyrimidine (Preparation 357) |
| | prep-HPLC (Method R, Gradient 25-55%) |
| | 11.5 mg, 11.0% yield as a white solid. LCMS m/z = 460.2 [M + H]⁺ |
| | 1H NMR (400 MHz, CDCl₃) δ ppm: 12.47 (s, 1H), 9.35 (s, 1H), 8.56 (s, |
| | 1H), 8.55 (s, 1H), 8.00 (s, 1H), 7.19 (s, 1H), 6.77 (s, 1H), 4.62 (t, J = 4.4 Hz, |
| | 2H), 3.78 (t, J = 4.8 Hz, 2H), 3.46 (s, 3H), 2.55 (s, 3H), 2.24 (s, 3H), 2.17 |
| | (t, J = 18.8 Hz, 3H). |
| 250 | N-(4-((2-(1,1-difluoroethyl)-6-methylpyrimidin-4-yl)amino)-5-(1-(2- |
| | methoxyethyl)-6-oxo-1,6-dihydropyrimidin-4-yl)pyridin-2-yl)acetamide |
| | 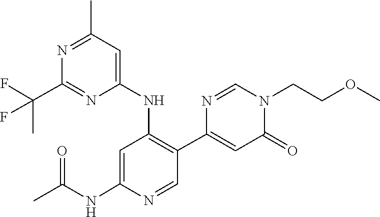 [SMILES: COCCn1cnc(-c2cnc(NC(C)=O)cc2Nc2cc(C)nc(C(C)(F)F)n2)cc1=O] |
| | SM: 6-chloro-3-(2-methoxyethyl)pyrimidin-4(3H)-one (Preparation 358) |
| | prep-HPLC (Method Y, Gradient 16-46%). 10.3 mg, 7.9% yield as a |
| | yellow solid. LCMS m/z = 460.0 [M + H]⁺ 1H NMR (400 MHz, DMSO-d₆) |
| | δ ppm: 11.31 (s, 1H), 10.60 (s, 1H), 9.04 (s, 1H), 8.61 (s, 1H), 8.56 (s, 1H), |
| | 7.01 (s, 1H), 6.89 (s, 1H), 4.11 (t, J = 5.2 Hz, 2H), 3.60 (t, J = 5.2 Hz, 2H), |
| | 3.27 (s, 3H), 2.41 (s, 3H), 2.12 (s, 3H), 2.04 (t, J = 18.4 Hz, 3H). |
| 251 | N-(4-((2-(1,1-difluoroethyl)-6-methylpyrimidin-4-yl)amino)-5- |
| | (pyridazin-3-yl)pyridin-2-yl)acetamide |
| | 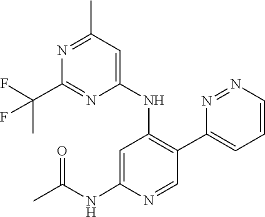 [SMILES: CC(=O)Nc1cc(Nc2cc(C)nc(C(C)(F)F)n2)c(-c2cccnn2)cn1] |
| | SM: 3-bromopyridazine |
| | Prep-HPLC (Method D, 21-31%). 19.0 mg, 16.9% yield as a white solid. |
| | LCMS m/z = 386.0 [M + H]⁺ ¹H NMR (400 MHz, DMSO-d₆) δ ppm 11.63 |
| | (s, 1H), 10.66 (s, 1H), 9.23 (dd, J = 4.8 Hz, 1.2 Hz, 1H), 9.09-9.06 (m, 1H), |
| | 8.73 (s, 1H), 8.27 (dd, J = 8.8 Hz, 1.6 Hz, 1H), 7.84 (dd, J = 8.8 Hz, 4.8 Hz, |
| | 1H), 6.95 (s, 1H), 2.42 (s, 3H), 2.15 (s, 3H), 2.03 (t, J = 19.2 Hz, 3H). |
| 252 | N-(4-((2-(1,1-difluoroethyl)-6-methylpyrimidin-4-yl)amino)-5-(5- |
| | methylpyridazin-3-yl)pyridin-2-yl)acetamide |
| | 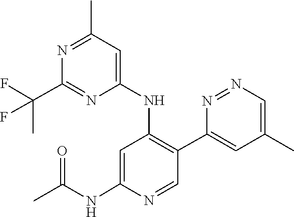 [SMILES: CC(=O)Nc1cc(Nc2cc(C)nc(C(C)(F)F)n2)c(-c2cc(C)cnn2)cn1] |
| | SM: 3-chloro-6-methylpyridazine |
| | Prep-HPLC (Method D, 25-55%). 9.5 mg, 8.4% yield as a brown solid. |
| | LCMS m/z = 400.1 [M + H]⁺ 1H NMR (400 MHz, CDCl₃) δ ppm: 13.19 (s, |
| | 1H), 9.71-9.80 (m, 2H), 9.07 (s, 1H), 8.56 (s, 1H), 7.84 (s, 1H), 6.89 (s, |
| | 1H), 2.53-2.57 (m, 6H), 2.24 (s, 3H), 2.19 (t, J = 18.8 Hz, 3H). |
| 253 | N-(4-((2-(1,1-difluoroethyl)-6-methylpyrimidin-4-yl)amino)-5-(5- |
| | (dimethylamino)pyridazin-3-yl)pyridin-2-yl)acetamide |
| | 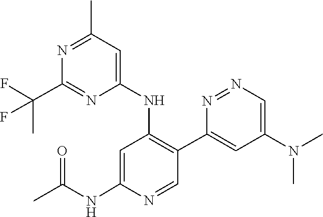 [SMILES: CC(=O)Nc1cc(Nc2cc(C)nc(C(C)(F)F)n2)c(-c2cc(N(C)C)cnn2)cn1] |
| | SM: 6-chloro-N,N-dimethylpyridazin-4-amine |
| | prep-HPLC (Method R, Gradient 22-52%). 8.1 mg, 1.6% yield as a dark |
| | red solid. LCMS m/z = 429.2 [M + H]⁺ 1H NMR (400 MHz, CDCl₃) δ ppm |
| | 12.71 (s, 1H), 9.48 (s, 1H), 8.68 (d, J = 2.8 Hz, 1H), 8.48 (s, 1H), 7.85 (s, |
| | 1H), 6.84 (d, J = 3.2 Hz, 1H), 6.78 (s, 1H), 3.18 (s, 6H), 2.50 (s, 3H), 2.15- |
| | 2.50 (m, 6H). |
| 254 | N-(4-((2-(1,1-difluoroethyl)-6-methylpyrimidin-4-yl)amino)-5-(5- |
| | (isopropylamino)pyridazin-3-yl)pyridin-2-yl)acetamide |
| | 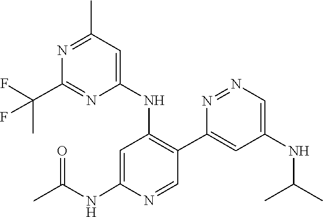 [SMILES: CC(=O)Nc1cc(Nc2cc(C)nc(C(C)(F)F)n2)c(-c2cc(NC(C)C)cnn2)cn1] |
| | SM: 6-bromo-N-isopropylpyridazin-4-amine |
| | prep-HPLC (Method R, Gradient 26-56%). 63.2 mg, 36.3% yield as a |
| | white solid. LCMS m/z = 443.2 [M + H]⁺ 1H NMR (400 MHz, CDCl₃) δ |
| | ppm 12.73 (s, 1H), 9.48 (s, 1H), 8.45-8.47 (m, 2H), 7.90 (s, 1H), 6.77- |
| | 6.81 (m 2H), 4.40 (d, J = 8.0 Hz, 1H), 3.76-3.86 (m, 1H), 2.50 (s, 3H), |
| | 2.25-2.15 (m, 6H), 1.34 (d, J = 6.4 Hz, 6H). |

### Example 255

### N-(4-((2-(1,1-difluoroethyl)pyrimidin-4-yl)amino)-5-(5-morpholino-1,3,4-thiadiazol-2-yl)pyridin-2-yl)acetamide

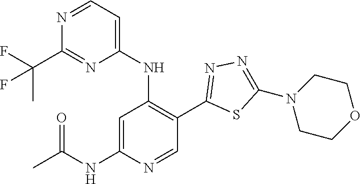 [SMILES: CC(=O)Nc1cc(Nc2ccnc(C(C)(F)F)n2)c(-c2nnc(N3CCOCC3)s2)cn1]

N-(4-((2-(1,1-difluoroethyl)pyrimidin-4-yl)amino)-5-(5-morpholino-1,3,4-thiadiazol-2-yl)pyridin-2-yl)acetamide was obtained as a white solid, 35 mg, 25.5%, from (6-acetamido-4-((2-(1,1-difluoroethyl)pyrimidin-4-yl)amino)pyridin-3-yl)boronic acid (Preparation 487) and 4-(5-bromo-1,3,4-thiadiazol-2-yl)morpholine, following a similar procedure to that described in Example 226. Prep-HPLC (Method R, Gradient 28-58%). LCMS m/z=463.2 [M+H]⁺ ¹H NMR (400 MHz, DMSO-d₆) δ: ppm 11.37 (s, 1H), 10.68 (s, 1H), 9.25 (s, 1H), 8.61 (d, J=3.0 Hz, 1H), 8.51 (s, 1H), 7.17 (s, 1H), 3.75-3.77 (m, 4H), 3.52-3.55 (m, 4H), 2.05-2.15 (m, 6H).

### Example 256

### N-(4-((2-(1,1-difluoroethyl)pyrimidin-4-yl)amino)-5-(6-(dimethylamino)pyrimidin-4-yl)pyridin-2-yl)acetamide

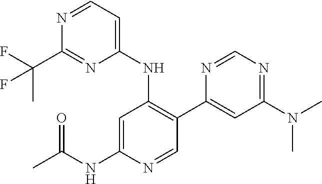 [SMILES: CC(=O)Nc1cc(Nc2ccnc(C(C)(F)F)n2)c(-c2cc(N(C)C)ncn2)cn1]

N-(4-((2-(1,1-difluoroethyl)pyrimidin-4-yl)amino)-5-(6-(dimethylamino)pyrimidin-4-yl)pyridin-2-yl)acetamide was obtained as a white solid, 12 mg, 9.8%, from (6-acetamido-4-((2-(1,1-difluoroethyl)pyrimidin-4-yl)amino)pyridin-3-yl)boronic acid (Preparation 487) and 6-chloro-N,N-dimethylpyrimidin-4-amine, following a similar procedure to that described in Example 226. Prep-HPLC (Method R, Gradient 29-59%) (12.0 mg, 9.8% yield) as a white solid. LCMS m/z=415.1 [M+H]⁺ 1H NMR (400 MHz, CDCl₃) δ ppm: 12.84 (s, 1H), 9.36 (s, 1H), 8.66 (s, 1H), 8.55 (s, 1H), 8.52 (d, J=5.6 Hz, 1H), 7.93 (s, 1H), 6.87 (d, J=6.0 Hz, 1H), 6.73 (s, 1H), 3.21 (s, 6H), 2.25 (s, 3H), 2.14-2.23 (t, J=18.8 Hz, 3H).

### Example 257 and 258

### N-(4-((2-(1,1-difluoroethyl)-6-methylpyrimidin-4-yl)amino)-5-(1-(2-methoxyethyl)-1H-pyrazol-3-yl)pyridin-2-yl)acetamide and N-(4-((2-(1,1-difluoroethyl)-6-methylpyrimidin-4-yl)amino)-5-(1-(2-methoxyethyl)-1H-pyrazol-5-yl)pyridin-2-yl)acetamide

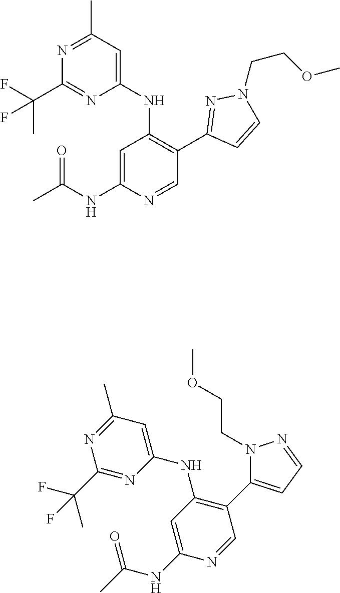 [SMILES: COCCn1ccc(-c2cnc(NC(C)=O)cc2Nc2cc(C)nc(C(C)(F)F)n2)n1 ; COCCn1nccc1-c1cnc(NC(C)=O)cc1Nc1cc(C)nc(C(C)(F)F)n1]

To a solution of (6-acetamido-4-((2-(1,1-difluoroethyl)-6-methylpyrimidin-4-yl)amino)pyridin-3-yl)boronic acid (Preparation 488, 400 mg, crude) in dioxane (3 mL) and water (0.6 mL) was added Pd(dppf)Cl₂-DCM (23.26 mg, 28.5 umol), K₂CO₃ (118.09 mg, 0.854 mmol), 3-bromo-1-(2-methoxymethyl)-1H-pyrazole (116.80 mg, 0.57 mmol) and the mixture was stirred at 90° C. for 2 h under N₂. The mixture was concentrated and purified by Prep-HPLC (Method R, Gradient 35-65%) to give N-(4-((2-(1,1-difluoroethyl)-6-methylpyrimidin-4-yl)amino)-5-(1-(2-methoxyethyl)-1H-pyrazol-3-yl)pyridin-2-yl)acetamide (20 mg, 16.3% yield) as a white solid. LCMS m/z=432.2 [M+H]⁺ ¹H NMR (400 MHz, CDCl₃) δ: ppm 8.95 (s, 1H), 8.07 (s, 1H), 7.93 (s, 1H), 7.70 (d, J=1.6 Hz, 1H), 7.45 (s, 1H), 6.75 (s, 1H), 6.29 (d, J=1.6 Hz, 1H), 4.09 (t, J=5.2 Hz, 2H), 3.82 (s, 2H), 3.31 (s, 3H), 2.51 (s, 3H), 2.25 (s, 3H), 2.08 (t, J=18.4 Hz, 3H).

and N-(4-((2-(1,1-difluoroethyl)-6-methylpyrimidin-4-yl)amino)-5-(1-(2-methoxyethyl)-1H-pyrazol-5-yl)pyridin-2-yl)acetamide (18 mg, 14.7% yield) as a grey solid. LCMS m/z=432.2 [M+H]⁺ ¹H NMR (400 MHz, CDCl₃) δ: ppm 11.34 (s, 1H), 9.28 (s, 1H), 8.48 (s, 1H), 7.94 (s, 1H), 7.57 (d, J=2.4 Hz, 1H), 6.79 (s, 1H), 6.64 (d, J=2.0 Hz, 1H), 4.40 (t, J=5.0 Hz, 2H), 3.85 (t, J=5.0 Hz, 2H), 3.38 (s, 3H), 2.55 (s, 3H), 2.06-2.28 (m, 6H).

### Example 259 and 260

### N-(4-((2-(1,1-difluoroethyl)-6-methylpyrimidin-4-yl)amino)-5-(1-(2-methoxyethyl)-1H-1,2,3-triazol-4-yl)pyridin-2-yl)acetamide and N-(4-((2-(1,1-difluoroethyl)-6-methylpyrimidin-4-yl)amino)-5-(2-(2-methoxyethyl)-2H-1,2,3-triazol-4-yl)pyridin-2-yl)acetamide

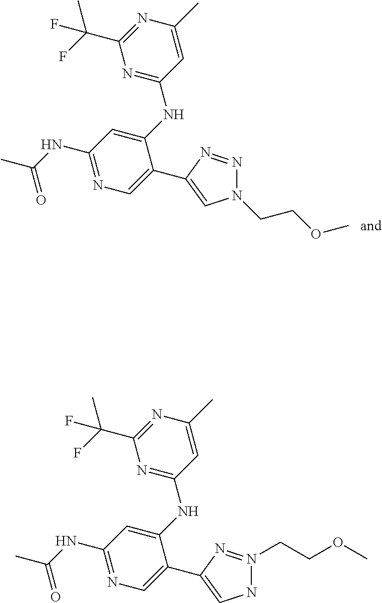

To a solution of (6-acetamido-4-((2-(1,1-difluoroethyl)-6-methylpyrimidin-4-yl)amino)pyridin-3-yl)boronic acid (Preparation 488, 150 mg, 0.43 mmol) in dioxane (3 mL) and H₂O (0.6 mL) was added a mixture of 4-bromo-2-(2-methoxyethyl)-2H-1,2,3-triazole and 4-bromo-1-(2-methoxyethyl)-1H-1,2,3-triazole (Preparation 290, 105.6 mg, 0.51 mmol), K₂CO₃ (118 mg, 0.85 mmol), Pd(dppf)Cl₂·DCM (34.9 mg, 0.043 mmol) and the resulting mixture stirred at 100° C. for 3 h under N₂. The mixture was evaporated to dryness and the residue purified by prep-HPLC (Method R, Gradient 28-58%) to give the title compounds. The structures were determined by 2D NMR.

Peak 1, Example 259. N-(4-((2-(1,1-difluoroethyl)-6-methylpyrimidin-4-yl)amino)-5-(1-(2-methoxyethyl)-1H-1,2,3-triazol-4-yl)pyridin-2-yl)acetamide as a white solid, 6.6 mg, 3.6%. LCMS m/z=433.1 [M+H]⁺; ¹H NMR (400 MHz, CDCl₃) δ ppm: 11.69 (s, 1H), 9.67 (s, 1H), 9.08 (br s, 1H), 8.36 (s, 1H), 8.11 (s, 1H), 6.87 (s, 1H), 4.66 (t, J=4.8 Hz, 2H), 3.83 (t, J=4.8 Hz, 2H), 3.41 (s, 3H), 2.56 (s, 3H), 2.28 (s, 3H), 2.20 (t, J=18.8 Hz, 3H).

Peak 2, Example 260. N-(4-((2-(1,1-difluoroethyl)-6-methylpyrimidin-4-yl)amino)-5-(2-(2-methoxyethyl)-2H-1,2,3-triazol-4-yl)pyridin-2-yl)acetamide as a white solid, 35 mg, 19%. LCMS m/z=433.2 [M+H]⁺; ¹H NMR (400 MHz, CDCl₃) δ ppm: 10.73 (br s, 1H), 9.59 (s, 1H), 8.42 (s, 1H), 7.98 (s, 1H), 6.87 (s, 1H), 4.75 (t, J=4.8 Hz, 2H), 3.98 (t, J=4.8 Hz, 2H), 3.40 (s, 3H), 2.60 (s, 3H), 2.31 (s, 3H), 2.18 (t, J=18.8 Hz, 3H).

### Example 261 and 262

### N-(4-((2-(1,1-difluoroethyl)-6-ethylpyrimidin-4-yl)amino)-5-(2-(2-methoxyethyl)-2H-1,2,3-triazol-4-yl)pyridin-2-yl)acetamide and N-(4-((2-(1,1-difluoroethyl)-6-ethylpyrimidin-4-yl)amino)-5-(1-(2-methoxyethyl)-1H-1,2,3-triazol-4-yl)pyridin-2-yl)acetamide

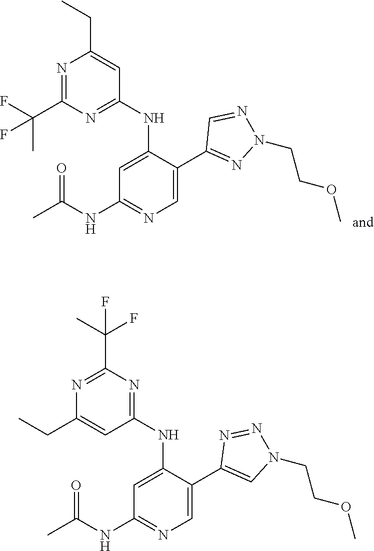

The title compounds were prepared from (6-acetamido-4-((2-(1,1-difluoroethyl)-6-ethylpyrimidin-4-yl)amino)pyridin-3-yl)boronic acid (Preparation 490) using an analogous method to that described for Example 259 and 260. The structures were determined by 2D NMR.

Peak 1, Example 261. N-(4-((2-(1,1-difluoroethyl)-6-ethylpyrimidin-4-yl)amino)-5-(2-(2-methoxyethyl)-2H-1,2,3-triazol-4-yl)pyridin-2-yl)acetamide LCMS m/z=447.2 [M+H]⁺; ¹H NMR (500 MHz, CDCl₃) δ ppm: 10.33 (s, 1H), 9.24 (s, 1H), 8.50 (s, 1H), 7.96 (s, 2H), 6.87 (s, 1H), 4.73 (t, J=5.5 Hz, 2H), 3.98 (t, J=5.5 Hz, 2H), 3.39 (s, 3H), 2.83 (q, J=6.4 Hz, 2H), 2.24 (s, 3H), 2.16 (t, J=19.0 Hz, 3H), 1.35 (t, J=8.0 Hz, 3H).

Peak 2, Example 262. N-(4-((2-(1,1-difluoroethyl)-6-ethylpyrimidin-4-yl)amino)-5-(1-(2-methoxyethyl)-1H-1,2,3-triazol-4-yl)pyridin-2-yl)acetamide. LCMS m/z=447.2 [M+H]⁺; ¹H NMR (500 MHz, CDCl₃) δ ppm: 11.36 (s, 1H), 9.49 (s, 1H), 8.38 (s, 1H), 8.04 (s, 1H), 7.85 (s, 1H), 6.82 (s, 1H), 4.64 (t, J=5.0 Hz, 2H), 3.83 (t, J=5.0 Hz, 2H), 3.41 (s, 3H), 2.81 (q, J=7.5 Hz, 2H), 2.24 (s, 3H), 2.20 (t, J=18.5 Hz, 3H), 1.33 (t, J=7.5 Hz, 3H).

### Example 263

### N-(4-((2-(1,1-difluoroethyl)-6-methylpyrimidin-4-yl)amino)-5-(1-(2-methoxyethyl)-1H-1,2,4-triazol-3-yl)pyridin-2-yl)acetamide

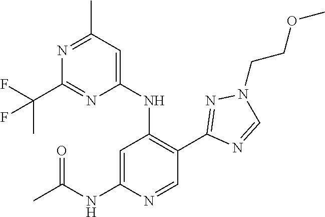 [SMILES: COCCn1cnc(-c2cnc(NC(C)=O)cc2Nc2cc(C)nc(C(C)(F)F)n2)n1]

To a solution of (6-acetamido-4-((2-(1,1-difluoroethyl)-6-methylpyrimidin-4-yl)amino)pyridin-3-yl)boronic acid (Preparation 488, 100 mg, 0.285 mmol) in dioxane (5 mL) and H₂O (1 mL) was added 3-bromo-1-(2-methoxyethyl)-1H-1,2,4-triazole (Preparation 291, 117.4 mg, 0.57 mmol), Pd(dppf)Cl₂-DCM (23.3 mg, 28.5 umol) and K₃PO₄ (120.9 mg, 0.57 mmol) and the reaction mixture was stirred at 100° C. for 16 h under N₂. The mixture was concentrated and was purified by Prep-HPLC (Method M, Gradient 19-39%) to give N-(4-((2-(1,1-difluoroethyl)-6-methylpyrimidin-4-yl)amino)-5-(1-(2-methoxyethyl)-1H-1,2,4-triazol-3-yl)pyridin-2-yl)acetamide (18 mg, 14.6% yield) as a yellow solid. LCMS m/z=433.1 [M+H]⁺ ¹H NMR (400 MHz, CDCl₃) δ: ppm 11.37 (s, 1H), 9.38 (s, 1H), 9.03 (s, 1H), 8.26 (s, 1H), 8.08 (s, 1H), 6.81 (s, 1H), 4.43 (t, J=4.8 Hz, 2H), 3.81 (t, J=4.8 Hz, 2H), 3.38 (s, 3H), 2.55 (s, 3H), 2.15-2.24 (m, 6H).

### Example 264

### N-(4-((2-(1,1-difluoroethyl)-6-methylpyrimidin-4-yl)amino)-5-(6-(2-methoxypropan-2-yl)pyrimidin-4-yl)pyridin-2-yl)acetamide

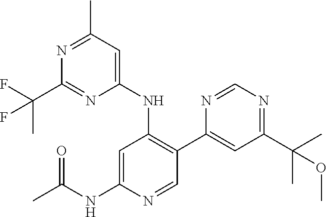 [SMILES: COC(C)(C)c1cc(-c2cnc(NC(C)=O)cc2Nc2cc(C)nc(C(C)(F)F)n2)ncn1]

N-(4-((2-(1,1-difluoroethyl)-6-methylpyrimidin-4-yl)amino)-5-(6-(2-methoxypropan-2-yl)pyrimidin-4-yl)pyridin-2-yl)acetamide was obtained as a white solid, 24.1 mg, 9.25%, from (6-acetamido-4-((2-(1,1-difluoroethyl)-6-methylpyrimidin-4-yl)amino)pyridin-3-yl)boronic acid (Preparation 488) and 4-chloro-6-(2-methoxypropan-2-yl)pyrimidine (Example 14, EP3036232B1), following a similar procedure to that described in Example 263. LCMS m/z=458.1 [M+H]⁺ ¹H NMR (400 MHz, DMSO-d6) δ ppm: 11.90 (s, 1H), 10.73 (s, 1H), 9.28 (s, 1H), 9.11 (d, J=4.8 Hz, 1H), 8.86 (s, 1H), 7.99 (s, 1H), 7.05 (s, 1H), 3.12 (s, 3H), 2.42 (s, 3H), 2.14 (s, 3H), 2.02 (t, J=19.6 Hz, 3H), 1.47 (s, 6H).

### Example 265

### N-(4′-((2-(1,1-difluoroethyl)pyrimidin-4-yl)amino)-5-((difluoromethoxy)methyl)-[2,3′-bipyridin]-6′-yl)acetamide

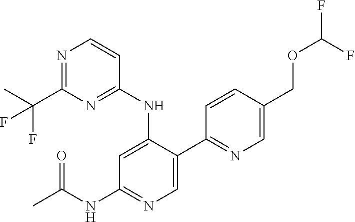 [SMILES: CC(=O)Nc1cc(Nc2ccnc(C(C)(F)F)n2)c(-c2ccc(COC(F)F)cn2)cn1]

A mixture of N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 125 mg, 0.179 mmol), 2-chloro-5-(difluoromethoxymethyl)pyrazine (Preparation 286, 52 mg, 0.268 mmol), Pd(PCy₃)₂ (11 mg, 17 mol) and aq. K₂CO₃ (2 M, 536.7 mol, 0.268 mL) in dioxane (2 mL) was heated at 100° C. for 2 h. The cooled mixture was filtered, the filtrate diluted with EtOAc, washed with water and brine. The organic layer was dried and concentrated and the crude product purified by HPLC (Method U, Gradient 5-95%) to give N-(4′-((2-(1,1-difluoroethyl)pyrimidin-4-yl)amino)-5-((difluoromethoxy)methyl)-[2,3′-bipyridin]-6′-yl)acetamide (13 mg, 16% yield). LCMS m/z=451 [M+H]+

### Example 266

### N-(4′-((2-(1,1-difluoroethyl)-6-ethylpyrimidin-4-yl)amino)-5-((dimethylamino)methyl)-[2,3′-bipyridin]-6′-yl)acetamide

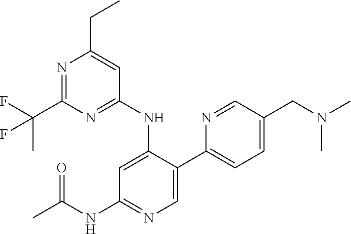 [SMILES: CCc1cc(Nc2cc(NC(C)=O)ncc2-c2ccc(CN(C)C)cn2)nc(C(C)(F)F)n1]

A mixture of N-(4-((2-(1,1-difluoroethyl)-6-ethylpyrimidin-4-yl)amino)-5-(4,4,5,5-tetramethyl-1,3,2-dioxaborolan-2-yl)pyridin-2-yl)acetamide (Preparation 491, 100 mg, 0.224 mmol), 1-(6-bromo-3-pyridyl)-N,N-dimethyl-methanamine (68.24 mg, 0.317 mmol), K₂CO₃ (100 mg, 724 mmol), XPhos Pd(crotyl)C1 (10.00 mg, 14.8 mol), dioxane (1 mL) and water (1 mL) was sealed and heated to 90° C. for 20 h. The cooled mixture was diluted with water (3 mL) and extracted with EtOAc (3×3 mL) and the combined organic extracts concentrated to dryness. The crude was purified by HPLC (Method U, Gradient 5-95%) to give N-(4′-((2-(1,1-difluoroethyl)-6-ethylpyrimidin-4-yl)amino)-5-((dimethylamino)methyl)-[2,3′-bipyridin]-6′-yl)acetamide (37.7 mg, 37% yield). LCMS m/z=456.2 [M+H]⁺. ¹H NMR (600 MHz, DMSO-d₆) δ (ppm)=12.86 (s, 1H), 10.54 (s, 1H), 9.24 (s, 1H), 8.80 (s, 1H), 8.72 (d, J=1.9 Hz, 1H), 8.07 (d, J=8.4 Hz, 1H), 7.94-7.82 (m, 1H), 7.11 (s, 1H), 3.51 (br s, 2H), 2.71 (d, J=7.6 Hz, 2H), 2.21 (s, 6H), 2.16-2.05 (m, 6H), 1.25 (s, 3H).

### Example 267

### N-(4′-((2-(1,1-difluoroethyl)-6-ethylpyrimidin-4-yl)amino)-5-morpholino-[2,3′-bipyridin]-6′-yl)acetamide

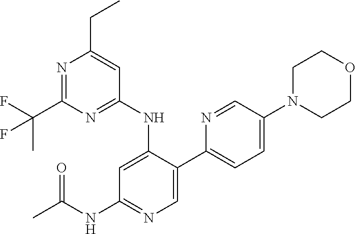 [SMILES: CCc1cc(Nc2cc(NC(C)=O)ncc2-c2ccc(N3CCOCC3)cn2)nc(C(C)(F)F)n1]

N-(4′-((2-(1,1-difluoroethyl)-6-ethylpyrimidin-4-yl)amino)-5-morpholino-[2,3′-bipyridin]-6′-yl)acetamide was obtained, 120 mg, 89%, from N-(4-((2-(1,1-difluoroethyl)-6-ethylpyrimidin-4-yl)amino)-5-(4,4,5,5-tetramethyl-1,3,2-dioxaborolan-2-yl)pyridin-2-yl)acetamide (Preparation 491) and 4-(6-bromo-3-pyridyl)morpholine, following a similar procedure to that described in Example 266. LCMS m/z=485 [M+H]⁺. ¹H NMR (500 MHz, DMSO-d₆) δ (ppm)=12.93 (s, 1H), 10.48 (s, 1H), 9.23 (s, 1H), 8.72 (s, 1H), 8.50 (d, J=2.7 Hz, 1H), 7.97 (d, J=9.2 Hz, 1H), 7.57 (dd, J=3.1, 9.2 Hz, 1H), 7.05 (s, 1H), 3.84-3.77 (m, 4H), 3.31-3.25 (m, 4H), 2.71 (q, J=7.6 Hz, 2H), 2.17-2.05 (m, 6H), 1.26 (t, J=7.6 Hz, 3H).

### Example 268

### N-(4′-((2-(1,1-difluoroethyl)-6-ethylpyrimidin-4-yl)amino)-5-(morpholinomethyl)-[2,3′-bipyridin]-6′-yl)acetamide

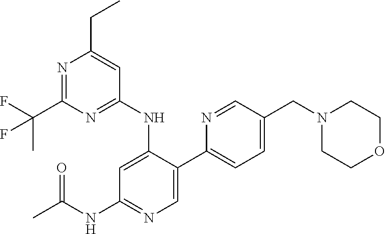 [SMILES: CCc1cc(Nc2cc(NC(C)=O)ncc2-c2ccc(CN3CCOCC3)cn2)nc(C(C)(F)F)n1]

A mixture of 2-chloro-5-(chloromethyl)pyridine (32 mg, 0.20 mmol), K₂CO₃ (83 mg, 0.60 mmol), 2-MeTHF (2.0 mL), morpholine (26 mg, 0.30 mmol), DMSO (0.25 mL) and DIPEA (26 mg, 0.20 mmol) was stirred for 20 h at 50° C. The reaction was concentrated to remove solvent, then N-(4-((2-(1,1-difluoroethyl)-6-ethylpyrimidin-4-yl)amino)-5-(4,4,5,5-tetramethyl-1,3,2-dioxaborolan-2-yl)pyridin-2-yl)acetamide (Preparation 491, 45 mg, 0.10 mmol) and XPhos Pd(crotyl)C1 (7 mg, 10 mmol) were added followed by 2-MeTHF (2.0 mL) and water (1.0 mL). The reaction was heated to 90° C. for 20 h, then diluted with water (3 mL) and extracted with 3:1 EtOAc:EtOH (3×3 mL). The combined organic layers were concentrated to dryness and purified via prep-HPLC (Method U, Gradient 5-95%) to give N-(4′-((2-(1,1-difluoroethyl)-6-ethylpyrimidin-4-yl)amino)-5-(morpholinomethyl)-[2,3′-bipyridin]-6′-yl)acetamide (10 mg, 20.11% yield). LCMS m/z=498.2 (M+H)+. ¹H NMR (500 MHz, DMSO-d₆) δ (ppm)=12.83 (s, 1H), 12.18-11.62 (m, 1H), 10.55 (s, 1H), 9.24 (s, 1H), 8.80 (s, 1H), 8.73 (s, 1H), 8.08 (d, J=8.2 Hz, 1H), 7.91 (dd, J=2.0, 8.4 Hz, 1H), 7.12 (s, 1H), 3.60 (br t, J=4.3 Hz, 4H), 3.58 (s, 2H), 2.46-2.38 (m, 4H), 2.17-2.03 (m, 9H).

### Example 269

### N-(4-((2-(1,1-difluoroethyl)-6-ethylpyrimidin-4-yl)amino)-5-(6-(2-methoxyethoxy)pyridazin-3-yl)pyridin-2-yl)acetamide trifluoroacetate

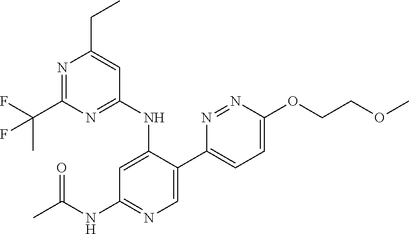 [SMILES: CCc1cc(Nc2cc(NC(C)=O)ncc2-c2ccc(OCCOC)nn2)nc(C(C)(F)F)n1]

A mixture of 3,6-dichloropyridazine (65 mg, 0.436 mmol), 2-methoxyethanol (65 mg, 0.854 mmol, 67.36 μL), 2-MeTHF (3 mL) and NaH (35 mg, 0.875 mmol, 60% purity) while stirring at rt. After 10 mins, the mixture was centrifuged, then mother liquor, clear solvent was passed through a syringe filter into another 8 mL vial. To this reaction mixture was added N-(4-((2-(1,1-difluoroethyl)-6-ethylpyrimidin-4-yl)amino)-5-(4,4,5,5-tetramethyl-1,3,2-dioxaborolan-2-yl)pyridin-2-yl)acetamide (Preparation 491, 100 mg, 0.223 mmol), K₂CO₃ (154.5 mg, 1.12 mmol), XPhos Pd(crotyl)C1 (7 mg, 10.4 mol) and water (2 mL). This reaction mixture was sealed and heated to 90° C. overnight, then diluted with 5 mL water and extracted 3×10 mL 3 EtOAc. The combined organic layers were concentrated to dryness and purified via prep-HPLC (Method V, Gradient 5-95%) to give N-(4-((2-(1,1-difluoroethyl)-6-ethylpyrimidin-4-yl)amino)-5-(6-(2-methoxyethoxy)pyridazin-3-yl)pyridin-2-yl)acetamide (69.3 mg, 66% yield). LCMS m/z=474.2 [M+H]⁺. ¹H NMR (600 MHz, DMSO-d₆) δ (ppm)=11.41 (s, 1H), 10.64 (s, 1H), 9.01 (s, 1H), 8.67 (s, 1H), 8.19 (d, J=9.5 Hz, 1H), 7.41 (d, J=9.2 Hz, 1H), 6.96 (s, 1H), 4.67-4.60 (m, 2H), 3.79-3.72 (m, 2H), 3.34 (s, 3H), 2.70 (q, J=7.6 Hz, 2H), 2.14 (s, 3H), 2.04 (t, J=19.3 Hz, 3H), 1.23 (t, J=7.6 Hz, 3H).

### Example 270

### N-(4-((2-(1,1-difluoroethyl)-6-methylpyrimidin-4-yl)amino)-5-(pyrimidin-4-yl)pyridin-2-yl)acetamide

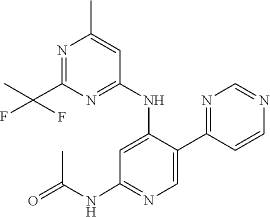 [SMILES: CC(=O)Nc1cc(Nc2cc(C)nc(C(C)(F)F)n2)c(-c2ccncn2)cn1]

A vial containing N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 448 mg, 1.0 mmol), 4-chloropyrimidine hydrochloride (240 mg, 1.6 mmol), PCy₃-Pd-G3 (90 mg, 0.123 mmol), and KOAc (1.5 M, 2.1 mL) in dioxane (5 mL) was degassed then backfilled with N₂ then heated at 95° C. 20 h. The cooled mixture was loaded onto a silica gel column and purified eluting with (20-70% 3:1 EtOAc:EtOH in heptane) The desired fractions were combined and concentrated under reduced pressure to afford an off-white solid that was suspended in EtOH and heated on the rotovap to 60° C. After 15 mins, the heterogeneous solution was filtered to afford N-(4-((2-(1,1-difluoroethyl)-6-methylpyrimidin-4-yl)amino)-5-(pyrimidin-4-yl)pyridin-2-yl)acetamide as an off-white solid (53 mg, 13% yield). LCMS m/z=386.1 [M+H]⁺. ¹H NMR (500 MHz, DMSO-d₆) δ (ppm)=12.28 (s, 1H), 10.67 (s, 1H), 9.32 (d, J=1.2 Hz, 1H), 9.25 (s, 1H), 8.90-8.86 (m, 2H), 8.16 (dd, J=1.2, 5.8 Hz, 1H), 7.12 (s, 1H), 2.44 (s, 3H), 2.14 (s, 3H), 2.07 (t, J=19.2 Hz, 3H).

### Example 271

### N-(4-((2-(1,1-difluoroethyl)-6-methylpyrimidin-4-yl)amino)-5-(2-(dimethylamino)thiazol-4-yl)pyridin-2-yl)acetamide

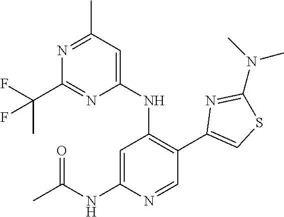 [SMILES: CC(=O)Nc1cc(Nc2cc(C)nc(C(C)(F)F)n2)c(-c2csc(N(C)C)n2)cn1]

To a solution of 4-bromo-N,N-dimethylthiazol-2-amine (Preparation 302, 100 mg, 0.483 mmol) in H₂O (0.8 mL), MeOH (4 mL) and toluene (4 mL) was added N-(5-bromo-4-((2-(1,1-difluoroethyl)-6-methylpyrimidin-4-yl)amino)pyridin-2-yl)acetamide (Preparation 483, 186.5 mg, 0.483 mmol), (BPin)₂ (245.2 mg, 0.967 mmol), CsF (440.1 mg, 2.90 mmol), cataCXium® A (69.3 mg, 0.193 mmol) and Pd(OAc)₂ (21.7 mg, 0.096 mmol). The resulting mixture was stirred at 100° C. for 16 h under N₂. The mixture was concentrated and purified by Prep-HPLC (Method K, Gradient 63-93%) to give N-(4-((2-(1,1-difluoroethyl)-6-methylpyrimidin-4-yl)amino)-5-(2-(dimethylamino)thiazol-4-yl)pyridin-2-yl)acetamide (7 mg, 3.3% yield) as a white solid. LCMS m/z=434.1 [M+H]⁺ ¹H NMR (400 MHz, CDCl₃) δ ppm: 12.38 (s, 1H), 9.39 (s, 1H), 8.26 (s, 1H), 6.89 (s, 1H), 6.77 (s, 1H), 3.27 (s, 6H), 2.59 (s, 3H), 2.33 (s, 3H), 2.13 (t, J=18.8 Hz, 3H).

### Examples 272 to 275

The compounds in the following table were prepared from N-(5-bromo-4-((2-(1,1-difluoroethyl)-6-methylpyrimidin-4-yl)amino)pyridin-2-yl)acetamide (Preparation 483) or N-(5-bromo-4-((2-(1,1-difluoroethyl)pyrimidin-4-yl)amino)pyridin-2-yl)acetamide (Preparation 484) and the appropriate halide, following a similar procedure to that described in Example 270.

| Example No | Name, Structure, Starting Material (SM), Data |
|---|---|
| 272 | N-(4-((2-(1,1-difluoroethyl)-6-methylpyrimidin-4-yl)amino)-5-(5-(2- |
| | hydroxypropan-2-yl)-1-methyl-1H-pyrazol-3-yl)pyridin-2-yl)acetamide |
| | 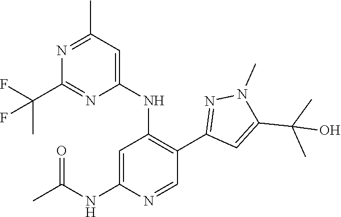 [SMILES: CC(=O)Nc1cc(Nc2cc(C)nc(C(C)(F)F)n2)c(-c2cc(C(C)(C)O)n(C)n2)cn1] |
| | SM: 2-(3-bromo-1-methyl-1H-pyrazol-5-yl)propan-2-ol (Preparation |
| | 292) and Preparation 483 |
| | prep-HPLC (Method Y, Gradient 19-49%). 18 mg, 5.9% yield as a white |
| | solid. LCMS m/z = 446.2 [M + H]⁺ ¹H NMR (400 MHz, MeOD-d₄) δ ppm: |
| | 9.30 (s, 1H), 8.54 ( s, 1H), 6.94 (s, 1H), 6.66 (s, 1H), 4.20 (s, 3H), 2.51 (s, |
| | 3H), 2.20 (s, 3H), 2.13 (t, J = 19.2 Hz, 3H), 1.67 (s, 6H). |
| 273 | N-(4-((2-(1,1-difluoroethyl)pyrimidin-4-yl)amino)-5-(6-methoxypyrazin- |
| | 2-yl)pyridin-2-yl)acetamide |
| | 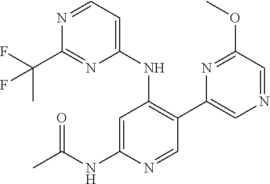 [SMILES: COc1cncc(-c2cnc(NC(C)=O)cc2Nc2ccnc(C(C)(F)F)n2)n1] |
| | SM: 2-chloro-6-methoxypyrazine and Preparation 484 |
| | prep-HPLC (Method R, Gradient 30-60%) 40.5 mg, 37.6% yield as a |
| | white solid. LCMS m/z = 402.1 [M + H]⁺ ¹H NMR (400 MHz, DMSO-d₆) |
| | δ ppm: 10.67 (s, 1H), 10.38 (s, 1H), 8.87 (s, 1H), 8.63 (s, 1H), 8.59 (s, 1H), |
| | 8.51 (d, J = 6.0 Hz, 1H), 8.27 (s, 1H), 7.05 (d, J = 5.6 Hz, 1H), 3.94 (s, 3H), |
| | 2.13 (s, 3H), 1.97 (t, J = 19.2 Hz, 3H). |
| 274 | N-(4-((2-(1,1-difluoroethyl)pyrimidin-4-yl)amino)-5-(6-((2- |
| | methoxyethyl)amino)pyrazin-2-yl)pyridin-2-yl)acetamide |
| | 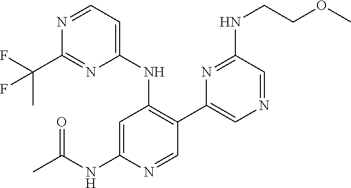 [SMILES: COCCNc1cncc(-c2cnc(NC(C)=O)cc2Nc2ccnc(C(C)(F)F)n2)n1] |
| | SM: 6-chloro-N-(2-methoxyethyl)pyrazin-2-amine (Preparation 359) and |
| | Preparation 484 |
| | Prep-HPLC (Method R, Gradient 30-60%) 47 mg, 13.3% yield as a yellow |
| | solid. LCMS m/z = 445.2 [M + H]⁺ ¹H NMR (400 MHz, CDCl₃) δ ppm: |
| | 12.01 (s, 1H), 8.73 (s, 1H), 8.58-8.60 (m, 2H), 8.24 (s, 1H), 8.03 (br s, 1H), |
| | 7.94 (s, 1H), 7.26-7.27 (m, 1H), 5.23 (br s, 1H), 3.79-3.83 (m, 2H), 3.71- |
| | 3.75 (m, 2H), 3.44 (s, 3H), 2.26 (s, 3H), 2.06 (t, J = 18.5 Hz, 3H). |
| 275 | N-(4-((2-(1,1-difluoroethyl)pyrimidin-4-yl)amino)-5-(6-((2- |
| | methoxyethyl)(methyl)amino)pyrazin-2-yl)pyridin-2-yl)acetamide |
| | 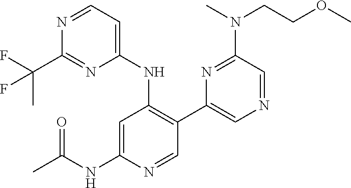 [SMILES: COCCN(C)c1cncc(-c2cnc(NC(C)=O)cc2Nc2ccnc(C(C)(F)F)n2)n1] |
| | SM: 6-chloro-N-(2-methoxyethyl)-N-methylpyrazin-2-amine |
| | (Preparation 360) and Preparation 484 |
| | Prep-HPLC (Method R, Gradient 25-55%) 46.4 mg, 13.6% yield as a |
| | yellow solid. LCMS m/z = 459.2 [M + H]⁺ ¹H NMR (500 MHz, CDCl₃) δ |
| | ppm: 11.74 (s, 1H), 8.78 (s, 1H), 8.57-8.60 (m, 2H), 8.23 (s, 1H), 8.10 (s, |
| | 1H), 8.00 (br s, 1H), 7.23-7.26 (m, 1H), 3.88 (t, J = 5.2 Hz, 2H), 3.70 (t, |
| | J = 5.2 Hz, 2H), 3.35 (s, 3H), 3.32 (s, 3H), 2.26 (s, 3H), 2.07 (t, J = 18.4 Hz, 3H) |

### Example 276

### N-(4′-((2-(1,1-difluoroethyl)pyrimidin-4-yl)amino)-5-(dimethylamino)-[2,3′-bipyridin]-6′-yl)acetamide

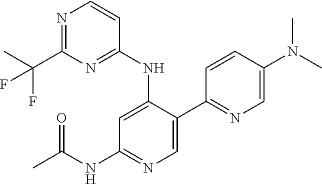 [SMILES: CC(=O)Nc1cc(Nc2ccnc(C(C)(F)F)n2)c(-c2ccc(N(C)C)cn2)cn1]

H₂O (0.2 mL), MeOH (0.8 mL) and THF (0.8 mL) were added to a mixture of N-(5-bromo-4-((2-(1,1-difluoroethyl)pyrimidin-4-yl)amino)pyridin-2-yl)acetamide (Preparation 484, 55 mg, 0.15 mmol), (BPin)₂ (76 mg, 0.30 mmol), 6-bromo-N,N-dimethylpyridin-3-amine (30 mg, 0.15 mmol), CsF (136 mg, 0.90 mmol), bis(1-adamantyl)-butyl-phosphane (32 mg, 0.090 mmol) and Pd(OAc)₂ (16 mg, 0.075 mmol). The resulting mixture was stirred at 100° C. for 1.5 h. The cooled reaction was diluted with EtOAc, washed with water and concentrated in vacuo. The residue was purified by HPLC to afford N-(4-((2-(1,1-difluoroethyl)-6-methylpyrimidin-4-yl)amino)-5-(1-ethyl-1H-pyrazol-3-yl)pyridin-2-yl)acetamide (1.6 mg, 2.5% yield) as a white powder. LCMS m/z=414 [M+H]⁺

### Example 277

### N-(4-((2-(1,1-difluoroethyl)-6-methylpyrimidin-4-yl)amino)-5-(5-methyl-4,5,6,7-tetrahydro-2H-pyrazolo[4,3-c]pyridin-2-yl)pyridin-2-yl)acetamide

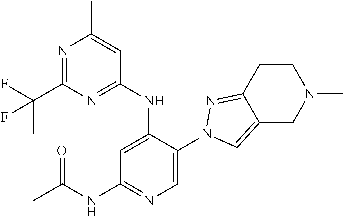 [SMILES: CC(=O)Nc1cc(Nc2cc(C)nc(C(C)(F)F)n2)c(-n2cc3c(n2)CCN(C)C3)cn1]

5-Methyl-2,4,6,7-tetrahydropyrazolo[4,3-c]pyridine (20 mg, 0.146 mmol) was added to a solution of N-(4-((2-(1,1-difluoroethyl)-6-methylpyrimidin-4-yl)amino)-5-(4,4,5,5-tetramethyl-1,3,2-dioxaborolan-2-yl)pyridin-2-yl)acetamide (Preparation 486, 69.5 mg, 0.16 mmol), Cu(OAc)₂ (29.3 mg, 0.160 mmol) and DMAP (35.6 mg, 0.292 mmol) in MeCN (1.1 mL). The reaction mixture was stirred overnight at rt open to air. Additional Cu(OAc)₂ (29.13 mg, 0.160 mmol) was added and the reaction mixture was heated at 40° C. for 8 h. The cooled reaction mixture was filtered through Celite®, evaporated and purified by HPLC (Method U, Gradient 5-95%) to afford N-(4-((2-(1,1-difluoroethyl)-6-methylpyrimidin-4-yl)amino)-5-(5-methyl-4,5,6,7-tetrahydro-2H-pyrazolo[4,3-c]pyridin-2-yl)pyridin-2-yl)acetamide (3.1 mg, 4.81% yield). LCMS m/z=443.2 [M+H]⁺

### Examples 278 and 279

### (R)-N-(4-((2-(1,1-difluoroethyl)-6-methylpyrimidin-4-yl)amino)-5-(tetrahydrofuran-2-yl)pyridin-2-yl)acetamide and (S)—N-(4-((2-(1,1-difluoroethyl)-6-methylpyrimidin-4-yl)amino)-5-(tetrahydrofuran-2-yl)pyridin-2-yl)acetamide

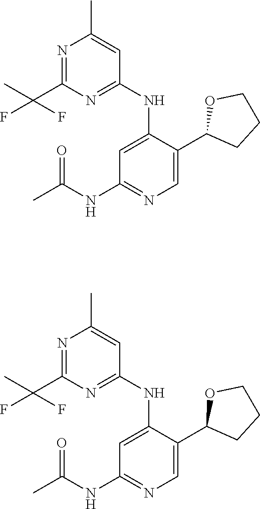 [SMILES: CC(=O)Nc1cc(Nc2cc(C)nc(C(C)(F)F)n2)c([C@H]2CCCO2)cn1 ; CC(=O)Nc1cc(Nc2cc(C)nc(C(C)(F)F)n2)c([C@@H]2CCCO2)cn1]

A vial containing N-(5-bromo-4-((2-(1,1-difluoroethyl)-6-methylpyrimidin-4-yl)amino)pyridin-2-yl)acetamide (Preparation 483, 325 mg, 0.842 mmol), tetrahydrofuran-2-carboxylic acid (0.13 mL, 1.35 mmol), Ir[dF(CF₃)ppy]₂(dtbpy)PF₆ (19 mg, 17 umol), and NiBr₂(dtbbpy) (26 mg, 45 umol) was taken into an inert glovebox, then 2-tert-butyl-1,1,3,3-tetramethyl-guanidine (0.26 mL, 1.29 mmol) and DMF (8 mL) were added. The vial was capped under a positive pressure of inert gas then sealed with electrical tape. The sealed vial was placed in a photoreactor and vigorously stirred at 23° C. with LED power set to 75%. After 20 h, the mixture was diluted with water and extracted with EtOAc (3×). The combined organics were dried (Na₂SO₄), filtered and concentrated under reduced pressure. The residue was purified by silica gel column chromatography (40-100% EtOAc in heptane) and the product further purified by HPLC (Method U, Gradient 5-60%), to afford a colorless film as N-(4-((2-(1,1-difluoroethyl)-6-methylpyrimidin-4-yl)amino)-5-(tetrahydrofuran-2-yl)pyridin-2-yl)acetamide (31 mg, 8% yield). This was further purified by SFC (Chiralpak IC 30×250 mm, 5 um column eluting with 30% EtOH with 0.1% DEA modifier at 100 mL/min; MBPR 40 psi, column temperature 40° C. to afford, the following compounds that were concentrated to dryness then lyophilized:

Peak 1, (R)-N-(4-((2-(1,1-difluoroethyl)-6-methylpyrimidin-4-yl)amino)-5-(tetrahydrofuran-2-yl)pyridin-2-yl)acetamide or (S)—N-(4-((2-(1,1-difluoroethyl)-6-methylpyrimidin-4-yl)amino)-5-(tetrahydrofuran-2-yl)pyridin-2-yl)acetamide (7 mg, 21% yield). LCMS m/z=378.1 [M+H]⁺. ¹H NMR (400 MHz, DMSO-d₆) δ (ppm)=10.44 (s, 1H), 9.17 (s, 1H), 8.60 (s, 1H), 8.22 (s, 1H), 6.92 (s, 1H), 5.06-5.00 (m, 1H), 4.09-4.03 (m, 1H), 3.84-3.77 (m, 1H), 2.39 (s, 3H), 2.31-2.24 (m, 1H), 2.07 (s, 3H), 2.04-1.94 (m, 5H), 1.74-1.64 (m, 1H).

And Peak 2, (S)—N-(4-((2-(1,1-difluoroethyl)-6-methylpyrimidin-4-yl)amino)-5-(tetrahydrofuran-2-yl)pyridin-2-yl)acetamide or (R)-N-(4-((2-(1,1-difluoroethyl)-6-methylpyrimidin-4-yl)amino)-5-(tetrahydrofuran-2-yl)pyridin-2-yl)acetamide (6 mg, 18% yield). LCMS m/z=378.1 (M+H)+. ¹H NMR (400 MHz, DMSO-d₆) δ (ppm)=10.44 (s, 1H), 9.17 (s, 1H), 8.60 (s, 1H), 8.22 (s, 1H), 6.92 (s, 1H), 5.05-5.00 (m, 1H), 4.09-4.03 (m, 1H), 3.84-3.78 (m, 1H), 2.39 (s, 3H), 2.31-2.23 (m, 1H), 2.07 (s, 3H), 2.05-1.93 (m, 5H), 1.72-1.65 (m, 1H).

### Example 280

### N-(4′-((2-(2-oxabicyclo[2.1.1]hexan-1-yl)-6-methylpyrimidin-4-yl)amino)-5-(methoxymethyl)-[2,3′-bipyridin]-6′-yl)acetamide

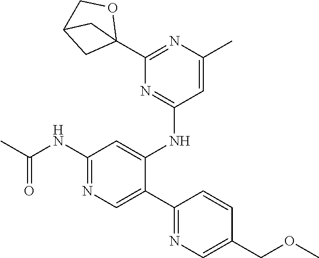 [SMILES: COCc1ccc(-c2cnc(NC(C)=O)cc2Nc2cc(C)nc(C34CC(CO3)C4)n2)nc1]

A mixture of N-(2-(2-oxabicyclo[2.1.1]hexan-1-yl)-6-methylpyrimidin-4-yl)-6′-chloro-5-(methoxymethyl)-[2,3′-bipyridin]-4′-amine (Preparation 504, 85 mg, 0.2 mmol), acetamide (59.22 mg, 1.0 mmol), Cs₂CO₃ (131 mg, 0.401 mmol) and BrettPhos Pd G3 (18 mg, 20 mol) in dioxane (2 mL) was heated at 100° C. for 1 h. The cooled mixture was filtered, the filtrate concentrated and the residue purified by chromatography on silica gel (0-100% EtOAc-EtOH 3:1 with 2% NH₄OH in heptane) to give N-(4′-((2-(2-oxabicyclo[2.1.1]hexan-1-yl)-6-methylpyrimidin-4-yl)amino)-5-(methoxymethyl)-[2,3′-bipyridin]-6′-yl)acetamide (58 mg, 64% yield) as a white solid. LCMS m/z=447 [M+H]⁺. ¹H NMR (MeOH-d₄, 400 MHz) δ 9.3-9.5 (m, 1H), 8.72 (s, 1H), 8.66 (s, 1H), 7.9-8.0 (m, 2H), 6.82 (d, 1H, J=0.8 Hz), 4.57 (s, 2H), 3.97 (s, 2H), 3.46 (s, 3H), 3.05 (t, 1H, J=3.3 Hz), 2.58 (ddd, 2H, J=1.9, 3.1, 4.6 Hz), 2.45 (s, 3H), 2.22 (s, 3H), 1.85 (dd, 2H, J=1.8, 4.8 Hz).

### Example 281

### N-(4-((2-(1,1-difluoroethyl)-6-ethylpyrimidin-4-yl)amino)-5-(3-methyl-1,2,4-oxadiazol-5-yl)pyridin-2-yl)acetamide

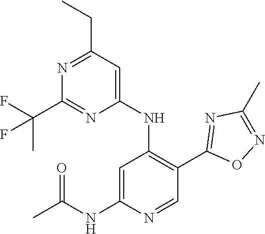 [SMILES: CCc1cc(Nc2cc(NC(C)=O)ncc2-c2nc(C)no2)nc(C(C)(F)F)n1]

To a solution of 6-acetamido-4-((2-(1,1-difluoroethyl)-6-ethylpyrimidin-4-yl)amino)nicotinic acid (Preparation 494, 100 mg, 0.274 mmol) in DMF (3 mL) was added HATU (104.1 mg, 0.274 mmol) and TEA (83.1 mg, 0.821 mmol) and the solution stirred at rt for 3 h. (Z)—N′-Hydroxyacetimidamide (60.8 mg, 0.821 mmol) was added and the reaction mixture was stirred at 110° C. for 4 h. The mixture was purified by Prep-HPLC (Method B, 37-67%) to give N-(4-((2-(1,1-difluoroethyl)-6-ethylpyrimidin-4-yl)amino)-5-(3-methyl-1,2,4-oxadiazol-5-yl)pyridin-2-yl)acetamide (46.3 mg, 41.9% yield) as a white solid. LCMS m/z=404.1 [M+H]⁺ 1H NMR (500 MHz, CDCl₃) δ ppm: 11.02 (s, 1H), 9.56 (s, 1H), 8.93 (s, 1H), 8.11 (s, 1H), 6.80 (s, 1H), 2.86 (q, J=7.0 Hz, 2H), 2.56 (s, 3H), 2.27 (s, 3H), 2.19 (t, J=19.0 Hz, 3H), 1.36 (t, J=7.5 Hz, 3H).

### Example 282

### N-(4-((2-(1,1-difluoroethyl)-6-methylpyrimidin-4-yl)amino)-5-(4,5,6,7-tetrahydropyrazolo[1,5-a]pyrazin-2-yl)pyridin-2-yl)acetamide

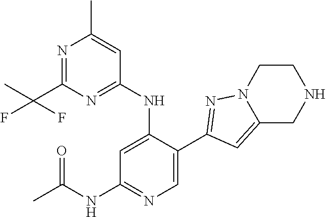 [SMILES: CC(=O)Nc1cc(Nc2cc(C)nc(C(C)(F)F)n2)c(-c2cc3n(n2)CCNC3)cn1]

To a vial containing tert-butyl 2-(6-acetamido-4-((2-(1,1-difluoroethyl)-6-methylpyrimidin-4-yl)amino)pyridin-3-yl)-6,7-dihydropyrazolo[1,5-a]pyrazine-5(4H)-carboxylate (Preparation 514, 100 mg, 0.189 mmol) in DCM (1 mL) was added TFA (0.15 mL, 2 mmol) and the reaction was stirred at 23° C. for 3 days. The reaction mixture was loaded onto a silica gel column and purified eluting with (20-100% EtOAc with 2% dimethylethylamine in heptane then 100% 3:1 EtOAc: EtOH). The product was further purified by HPLC (Method U, Gradient 5-50%) to give N-(4-((2-(1,1-difluoroethyl)-6-methylpyrimidin-4-yl)amino)-5-(4,5,6,7-tetrahydropyrazolo[1,5-a]pyrazin-2-yl)pyridin-2-yl)acetamide as a white solid (46 mg, 54% yield). LCMS m/z=429.2 [M+H]⁺. ¹H NMR (500 MHz, DMSO-d₆) δ (ppm)=11.57 (s, 1H), 10.43 (s, 1H), 9.33 (s, 1H), 8.63 (s, 1H), 7.03 (s, 1H), 6.67 (s, 1H), 4.19 (t, J=5.5 Hz, 2H), 3.97 (s, 2H), 3.19 (t, J=5.5 Hz, 2H), 2.98-2.67 (m, 1H), 2.46 (s, 3H), 2.18-2.10 (m, 6H).

### Example 283

### N-(4-((2-(1,1-difluoroethyl)-6-methylpyrimidin-4-yl)amino)-5-(5-ethyl-4,5,6,7-tetrahydropyrazolo[1,5-a]pyrazin-2-yl)pyridin-2-yl)acetamide

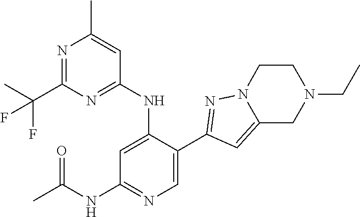 [SMILES: CCN1CCn2nc(-c3cnc(NC(C)=O)cc3Nc3cc(C)nc(C(C)(F)F)n3)cc2C1]

To a suspension of N-(4-((2-(1,1-difluoroethyl)-6-methylpyrimidin-4-yl)amino)-5-(4,5,6,7-tetrahydropyrazolo[1,5-a]pyrazin-2-yl)pyridin-2-yl)acetamide (Example 282, 40 mg, 0.093 mmol) in MeOH (1 mL) was added acetaldehyde (4.11 mg, 93.4 mol) followed by NaBH₃CN (1 M, 0.187 mmol, 0.187 mL) in THF and the reaction was stirred at rt for 1 h. The mixture was concentrated in vacuo and the residue purified by silica gel chromatography (0-5% MeOH in DCM) to give N-(4-((2-(1,1-difluoroethyl)-6-methylpyrimidin-4-yl)amino)-5-(5-ethyl-4,5,6,7-tetrahydropyrazolo[1,5-a]pyrazin-2-yl)pyridin-2-yl)acetamide as a white solid (22.8 mg, 50.8% yield). LCMS m/z=457.4 [M+H]⁺ 1H NMR (500 MHz, CDCl₃) δ ppm 11.35 (s, 1H), 9.35 (br s, 1H), 8.45 (s, 1H), 7.86 (s, 1H), 6.74 (s, 1H), 6.39 (s, 1H), 4.32 (t, J=5.5 Hz, 2H), 3.76 (s, 2H), 3.02 (t, J=5.5 Hz, 2H), 2.69 (q, J=7.3 Hz, 2H), 2.55 (s, 3H), 2.16-2.27 (m, 6H), 1.22 (t, J=7.2 Hz, 3H).

### Example 284

### N-(4-((2-(1,1-difluoroethyl)pyrimidin-4-yl)amino)-5-(5-methyl-5,6-dihydro-4H-pyrrolo[3,4-d]thiazol-2-yl)pyridin-2-yl)acetamide

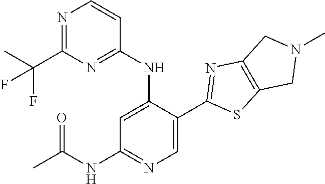 [SMILES: CC(=O)Nc1cc(Nc2ccnc(C(C)(F)F)n2)c(-c2nc3c(s2)CN(C)C3)cn1]

Step 1: To a vial was added tert-butyl 2-(6-acetamido-4-((2-(1,1-difluoroethyl)pyrimidin-4-yl)amino)pyridin-3-yl)-4,6-dihydro-5H-pyrrolo[3,4-d]thiazole-5-carboxylate (Preparation 515, 100 mg, 0.193 mmol), DCM (12.39 μL) and TFA (14.80 μL) and the reaction mixture was stirred at rt overnight. The organic layer was dried over Na₂SO₄ and solvent removed in vacuo to give N-(4-((2-(1,1-difluoroethyl)pyrimidin-4-yl)amino)-5-(5,6-dihydro-4H-pyrrolo[3,4-d]thiazol-2-yl)pyridin-2-yl)acetamide.

Step 2: A mixture of N-(4-((2-(1,1-difluoroethyl)pyrimidin-4-yl)amino)-5-(5,6-dihydro-4H-pyrrolo[3,4-d]thiazol-2-yl)pyridin-2-yl)acetamide (80 mg, 0.192 mmol), formaldehyde (155.5 mg, 1.92 mmol), MeOH (1.48 mL) and TEA (38.79 mg, 0.383 mmol) was stirred for 30 mins at rt. NaBH(OAc)₃ (203.1 mg, 0.958 mmol) and AcOH (34.53 mg, 575 mmol) were added and the reaction mixture was stirred at rt overnight. The organic layer was dried over Na₂SO₄ and solvent removed in vacuo. The crude product was purified by HPLC (Method U, Gradient 5-95%) to give N-(4-((2-(1,1-difluoroethyl)pyrimidin-4-yl)amino)-5-(5-methyl-5,6-dihydro-4H-pyrrolo[3,4-d]thiazol-2-yl)pyridin-2-yl)acetamide.

### Example 285

### N-(5-(5-(cyanomethyl)-4,5,6,7-tetrahydrothiazolo[5,4-c]pyridin-2-yl)-4-((2-(1,1-difluoroethyl)-6-methylpyrimidin-4-yl)amino)pyridin-2-yl)acetamide

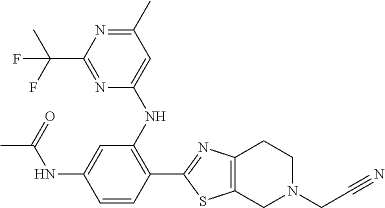 [SMILES: CC(=O)Nc1ccc(-c2nc3c(s2)CN(CC#N)CC3)c(Nc2cc(C)nc(C(C)(F)F)n2)c1]

To a solution of N-(4-((2-(1,1-difluoroethyl)-6-methylpyrimidin-4-yl)amino)-5-(4,5,6,7-tetrahydrothiazolo[5,4-c]pyridin-2-yl)pyridin-2-yl)acetamide hydrochloride (Preparation 519, 50 mg, 0.11 mmol) in MeCN (3 mL) was added K₂CO₃ (31 mg, 0.22 mmol) and bromoacetonitrile (80.8 mg, 0.67 mmol) and the mixture stirred at 25° C. for 16 h. The reaction was quenched by the addition of saturated aqueous NH₄Cl solution (20 mL) at 0° C. and evaporated to dryness under reduced pressure. The residue was purified by prep-HPLC (Method R, Gradient 37-67%) to give N-(5-(5-(cyanomethyl)-4,5,6,7-tetrahydrothiazolo[5,4-c]pyridin-2-yl)-4-((2-(1,1-difluoroethyl)-6-methylpyrimidin-4-yl)amino)pyridin-2-yl)acetamide as a white solid (12.8 mg, 23%). LCMS m/z=485.2 [M+H]⁺; ¹H NMR (500 MHz, CDCl₃) δ ppm: 12.32 (s, 1H), 9.56 (s, 1H), 8.53 (s, 1H), 8.38 (br s, 1H), 6.70 (s, 1H), 3.93 (s, 2H), 3.80 (s, 2H), 3.09-3.06 (m, 4H), 2.57 (s, 3H), 2.26 (s, 3H), 2.20 (t, J=19.0 Hz, 3H).

### Example 286

### N-(5-(5-cyanopyridazin-3-yl)-4-((2-(1,1-difluoroethyl)-6-methylpyrimidin-4-yl)amino)pyridin-2-yl)acetamide

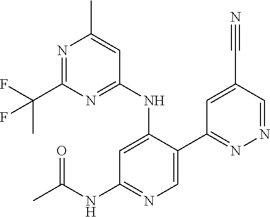 [SMILES: CC(=O)Nc1cc(Nc2cc(C)nc(C(C)(F)F)n2)c(-c2cc(C#N)cnn2)cn1]

Step 1: To a solution of (6-acetamido-4-((2-(1,1-difluoroethyl)-6-methylpyrimidin-4-yl)amino)pyridin-3-yl)boronic acid (Preparation 488, 200 mg, 0.57 mmol) in dioxane (5 mL) and H₂O (1 mL) was added 3,5-dichloropyridazine (170 mg, 1.14 mmol), Pd(dppf)Cl₂·DCM (46.5 mg, 0.057 mmol) and K₃PO₄ (242 mg, 1.14 mmol) and the mixture stirred at 100° C. for 6 h under N2. The mixture was concentrated and the residue purified by chromatography on silica gel (100% EtOAc) to give N-(5-(5-chloropyridazin-3-yl)-4-((2-(1,1-difluoroethyl)-6-methylpyrimidin-4-yl)amino)pyridin-2-yl)acetamide as a yellow solid (50 mg, 21%). ¹H NMR (400 MHz, CDCl₃) δ ppm: 12.31 (s, 1H), 9.53 (s, 1H), 9.14 (s, 1H), 8.52 (s, 1H), 8.04 (s, 1H), 8.01 (d, J=2.0 Hz, 1H), 6.81 (s, 1H), 2.54 (s, 3H), 2.14-2.26 (m, 6H).

Step 2. To a solution of N-(5-(5-chloropyridazin-3-yl)-4-((2-(1,1-difluoroethyl)-6-methylpyrimidin-4-yl)amino)pyridin-2-yl)acetamide (Step 1, 30 mg, 0.071 mmol) in DMF (1 mL) was added dppf (7.92 mg, 0.014 mmol), Pd₂(dba)₃ (6.54 mg, 0.072 mmol), Zn (4.67 mg, 0.071 mmol), Zn(CN)₂ (25.2 mg, 0.214 mmol) and the mixture stirred in the microwave at 130° C. for 2 h under N₂. The reaction mixture was concentrated and the residue purified by prep-HPLC (Method R, Gradient 26-56%) to give N-(5-(5-cyanopyridazin-3-yl)-4-((2-(1,1-difluoroethyl)-6-methylpyrimidin-4-yl)amino)pyridin-2-yl)acetamide as a yellow solid (15 mg, 51%). LCMS m/z=411.2 [M+H]⁺; ¹H NMR (400 MHz, CDCl₃) δ ppm: 12.06 (s, 1H), 9.53 (s, 1H), 9.30 (d, J=1.6 Hz, 1H), 8.55 (s, 1H), 8.22 (s, 1H), 7.93 (s, 1H), 6.83 (s, 1H), 2.55 (s, 3H), 2.27 (s, 3H), 2.18 (t, J=19.2 Hz, 3H).

### Example 287

### N-(4-((2-(1,1-difluoroethyl)-6-methylpyrimidin-4-yl)amino)-5-(5-((dimethylamino)methyl)-1-methyl-1H-pyrazol-3-yl)pyridin-2-yl)acetamide

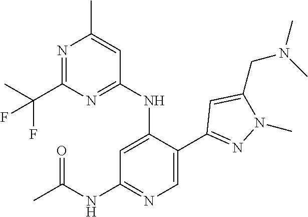 [SMILES: CC(=O)Nc1cc(Nc2cc(C)nc(C(C)(F)F)n2)c(-c2cc(CN(C)C)n(C)n2)cn1]

To a solution of N-(4-((2-(1,1-difluoroethyl)-6-methylpyrimidin-4-yl)amino)-5-(5-formyl-1-methyl-1H-pyrazol-3-yl)pyridin-2-yl)acetamide (Preparation 509, 25 mg, 0.060 mmol) in MeOH (2 mL) was added Me₂NH·HCl (5.4 mg, 0.066 mmol) and NaBH₃CN (18.9 mg, 0.300 mmol) and the mixture stirred at 70° C. for 16 h. The reaction mixture was concentrated and the residue purified by prep-HPLC (Method D, Gradient 0-30%) to give N-(4-((2-(1,1-difluoroethyl)-6-methylpyrimidin-4-yl)amino)-5-(5-((dimethylamino)methyl)-1-methyl-1H-pyrazol-3-yl)pyridin-2-yl)acetamide as a white solid (10 mg, 37%). LCMS m/z=445.2 [M+H]⁺; ¹H NMR (500 MHz, DMSO-d₆) δ ppm: 11.54 (s, 1H), 10.43 (s, 1H), 9.32 (s, 1H), 8.65 (s, 1H), 7.05 (s, 1H), 6.85 (s, 1H), 3.99 (s, 3H), 3.52 (s, 2H), 2.46 (s, 3H), 2.21-2.11 (m, 12H).

### Example 288

### 1-(4-((2-(1,1-difluoroethyl)-6-methylpyrimidin-4-yl)amino)-5-(1-methyl-1H-pyrazol-3-yl)pyridin-2-yl)urea

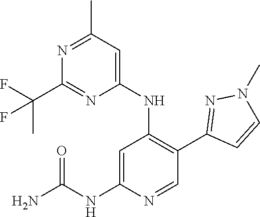 [SMILES: Cc1cc(Nc2cc(NC(N)=O)ncc2-c2ccn(C)n2)nc(C(C)(F)F)n1]

To a solution of N-(2-chloro-5-(1-methyl-1H-pyrazol-3-yl)pyridin-4-yl)-2-(1,1-difluoroethyl)-6-methylpyrimidin-4-amine (Preparation 501, 150 mg, 0.411 mmol) in dioxane (5 mL) was added urea (123.5 mg, 2.06 mmol), BrettPhos Pd G₃ (37.28 mg, 0.041 mmol) and K₃PO₄ (174.6 mg, 0.822 mmol) at 25° C. The mixture was stirred at 100° C. for 16 h under N₂. The mixture was concentrated and purified by prep-HPLC (Column: Phenomenex C18 150*25 mm*10 μm; Condition: water (NH₄HCO₃)-ACN; B %: 16%-46%, 10 min) to give 1-(4-((2-(1,1-difluoroethyl)-6-methylpyrimidin-4-yl)amino)-5-(1-methyl-1H-pyrazol-3-yl)pyridin-2-yl)urea (10.7 mg, 6.70% yield) as a white solid. LCMS m/z=389.2 [M+H]⁺ 1H NMR (400 MHz, DMSO-d₆) δ ppm 11.39 (s, 1H), 9.15 (s, 1H), 8.67 (s, 1H), 8.57 (s, 1H), 7.88 (d, J=2.4 Hz, 1H), 7.06 (s, 1H), 6.86 (d, J=2.4 Hz, 1H), 6.59-6.85 (m, 2H), 4.02 (s, 3H), 2.46 (s, 3H), 2.12 (t, J=19.2 Hz, 3H).

### Examples 289 to 290

The compounds in the following table were prepared from the appropriate chloride and urea, following a similar procedure to that described in Example 289.

| Example No | Name, Structure, Starting material (SM), Data |
|---|---|
| 289 | 1-(4-((2-(1,1-difluoroethyl)-6-methylpyrimidin-4-yl)amino)-5-(1-methyl- |
| | 1H-pyrazol-3-yl)pyridin-2-yl)-3-methylurea |
| | 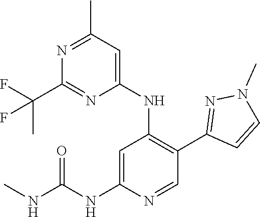 [SMILES: CNC(=O)Nc1cc(Nc2cc(C)nc(C(C)(F)F)n2)c(-c2ccn(C)n2)cn1] |
| | SM: N-(2-chloro-5-(1-methyl-1H-pyrazol-3-yl)pyridin-4-yl)-2-(1,1- |
| | difluoroethyl)-6-methylpyrimidin-4-amine (Preparation 501) and 1-methylurea |
| | prep-HPLC (Method Y, Gradient 14-44%). 10.2 mg, 6.2% yield as a white |
| | solid. LCMS m/z = 403.0 [M + H]⁺ ¹H NMR (400 MHz, DMSO-d₆) δ ppm: |
| | 11.35 (s, 1H), 9.35 (s, 1H), 8.55 (s, 1H), 8.40 (s, 1H), 7.98 (d, J = 3.6 Hz, |
| | 1H), 7.87 (d, J = 2.8 Hz, 1H), 7.06 (s, 1H), 6.84 (d, J = 2.4 Hz, 1H), 4.01 (s, |
| | 3H), 2.73 (d, J = 4.4 Hz, 3H), 2.46 (s, 3H), 2.11 (t, J = 19.2 Hz, 3H). |
| 290 | 1-(4′-((2-(1,1-difluoroethyl)-6-methylpyrimidin-4-yl)amino)-5-fluoro- |
| | [2,3′-bipyridin]-6′-yl)-3-methylurea |
| | 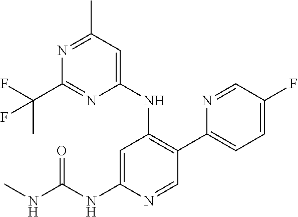 [SMILES: CNC(=O)Nc1cc(Nc2cc(C)nc(C(C)(F)F)n2)c(-c2ccc(F)cn2)cn1] |
| | SM: 6′-chloro-N-(2-(1,1-difluoroethyl)-6-methylpyrimidin-4-yl)-5- |
| | fluoro-[2,3′-bipyridin]-4′-amine (Preparation 503) and 1-methylurea |
| | Prep-HPLC (Method G, Gradient 36-66%). 10 mg, 9.1% yield as a white |
| | solid. LCMS m/z = 418.1 [M + H]⁺ ¹H NMR (400 MHz, CDCl₃) δ ppm: |
| | 12.21 (s, 1H), 9.30 (br s, 1H), 8.56 (d, J = 2.8 Hz, 1H), 8.45 (s, 1H), 8.23 (s, |
| | 1H), 7.78 (dd, J = 8.8 Hz, 4.0 Hz, 1H), 7.59-7.65 (m, 1H), 6.98 (br s, 1H), |
| | 6.60 (s, 1H), 2.98 (d, J = 4.8 Hz, 3H), 2.52 (s, 3H), 2.05 (t, J = 18.4 Hz, 3H). |

### Example 291

### 1-(4′-((2-(1,1-difluoroethyl)-6-methylpyrimidin-4-yl)amino)-5-fluoro-[2,3′-bipyridin]-6′-yl)urea

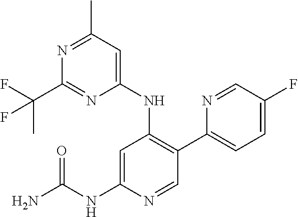 [SMILES: Cc1cc(Nc2cc(NC(N)=O)ncc2-c2ccc(F)cn2)nc(C(C)(F)F)n1]

To a solution of 6′-chloro-N-(2-(1,1-difluoroethyl)-6-methylpyrimidin-4-yl)-5-fluoro-[2,3′-bipyridin]-4′-amine (Preparation 503, 100 mg, 0.263 mmol) in DMF (3 mL) was added urea (79.1 mg, 1.32 mmol), K₃PO₄ (111.8 mg, 0.527 mmol) and BrettPhos Pd G₃ (23.9 mg, 26.3 umol). The mixture was stirred at 90° C. of 1 h under N₂ in microwave. The mixture was concentrated and was purified by Prep-HPLC (Method M, Gradient 20-50%) to give 1-(4′-((2-(1,1-difluoroethyl)-6-methylpyrimidin-4-yl)amino)-5-fluoro-[2,3′-bipyridin]-6′-yl)urea (25 mg, 23.5% yield) as a yellow solid. LCMS m/z=404.2 [M+H]⁺ ¹H NMR (400 MHz, DMSO-d₆) δ: ppm 11.75 (s, 1H), 9.27 (s, 1H), 8.74 (d, J=3.2 Hz, 1H), 8.59 (s, 1H), 8.53 (d, J=4.8 Hz, 1H), 8.02-8.05 (m, 1H), 7.89-7.92 (m, 1H), 7.04 (s, 1H), 6.87 (brs, 2H), 2.41 (s, 3H), 2.06 (t, J=19.2 Hz, 3H).

### Example 292

### 1-(5-cyano-4′-((2-(1,1-difluoroethyl)-6-methylpyrimidin-4-yl)amino)-[2,3′-bipyridin]-6′-yl)urea

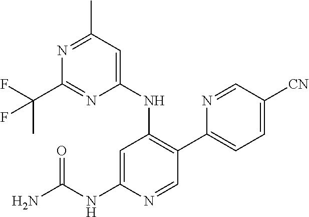 [SMILES: Cc1cc(Nc2cc(NC(N)=O)ncc2-c2ccc(C#N)cn2)nc(C(C)(F)F)n1]

1-(5-Cyano-4′-((2-(1,1-difluoroethyl)-6-methylpyrimidin-4-yl)amino)-[2,3′-bipyridin]-6′-yl)urea was obtained as a yellow solid, 10 mg, 7.9%, from 6′-chloro-4′-((2-(1,1-difluoroethyl)-6-methylpyrimidin-4-yl)amino)-[2,3′-bipyridine]-5-carbonitrile (Preparation 502) and urea, following the procedure described in Example 292. LCMS m/z=411.1 [M+H]⁺ ¹H NMR (400 MHz, DMSO-d₆) δ ppm: 11.86 (s, 1H), 9.36 (s, 1H), 9.15 (s, 1H), 8.72 (s, 1H), 8.60 (s, 1H), 8.40 (dd, J=8.4 Hz, 2.0 Hz, 1H), 8.16 (d, J=8.0 Hz, 1H), 7.07 (s, 1H), 6.86 (brs, 2H), 2.42 (s, 3H), 2.05 (t, J=19.2 Hz, 3H).

### Examples 293 to 519

The compounds in the table below were made using a similar process to the Examples described above.

| Ex. No. | Name/Structure, Data |
|---|---|
| 293 | (S)-N-(4-((2-(1,1-difluoroethyl)-6-methylpyrimidin-4-yl)amino)-5-((tetrahydrofuran-2- |
| | yl)methoxy)pyridin-2-yl)acetamide or (R)-N-(4-((2-(1,1-difluoroethyl)-6-methylpyrimidin- |
| | 4-yl)amino)-5-((tetrahydrofuran-2-yl)methoxy)pyridin-2-yl)acetamide |
| | 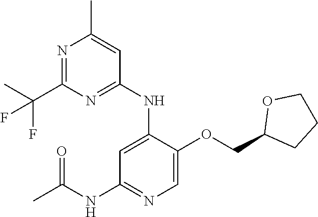 [SMILES: CC(=O)Nc1cc(Nc2cc(C)nc(C(C)(F)F)n2)c(OC[C@@H]2CCCO2)cn1] |
| | Or |
| | 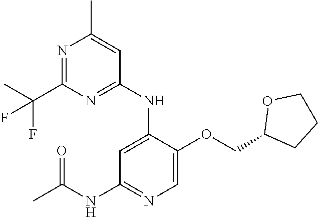 [SMILES: CC(=O)Nc1cc(Nc2cc(C)nc(C(C)(F)F)n2)c(OC[C@H]2CCCO2)cn1] |
| | LCMS m/z = 408.2 [M + H]⁺ |
| 294 | (R)-N-(4-((2-(1,1-difluoroethyl)-6-methylpyrimidin-4-yl)amino)-5-((tetrahydrofuran-2- |
| | yl)methoxy)pyridin-2-yl)acetamide or (S)-N-(4-((2-(1,1-difluoroethyl)-6-methylpyrimidin-4- |
| | yl)amino)-5-((tetrahydrofuran-2-yl)methoxy)pyridin-2-yl)acetamide |
| | 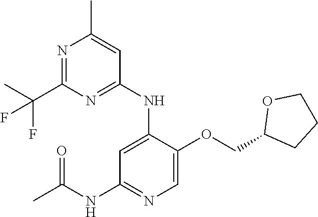 [SMILES: CC(=O)Nc1cc(Nc2cc(C)nc(C(C)(F)F)n2)c(OC[C@H]2CCCO2)cn1] |
| | Or |
| | 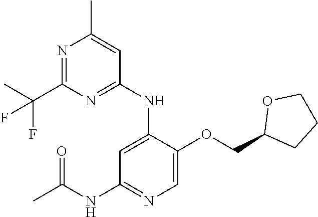 [SMILES: CC(=O)Nc1cc(Nc2cc(C)nc(C(C)(F)F)n2)c(OC[C@@H]2CCCO2)cn1] |
| | LCMS m/z = 408.2 [M + H]⁺. |
| 295 | N-(4-((2-(1,1-difluoroethyl)-6-methylpyrimidin-4-yl)amino)-5-(3-fluoropropoxy)pyridin-2- |
| | yl)acetamide |
| | 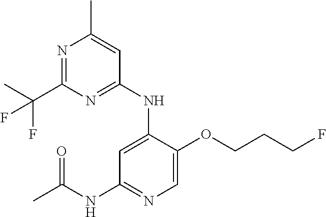 [SMILES: CC(=O)Nc1cc(Nc2cc(C)nc(C(C)(F)F)n2)c(OCCCF)cn1] |
| | LCMS m/z = 384.3 [M + H]⁺; ¹H NMR (600 MHz, DMSO-d₆) δ ppm 10.45 (br s, 1H), 9.17 |
| | (br s, 1H), 8.90 (br s, 1H), 8.02 (s, 1H), 7.16 (s, 1H), 4.59-4.72 (m, 2H), 4.22 (t, J = 6.1 |
| | Hz, 2H), 2.42 (s, 3H), 2.12-2.22 (m, 2H), 1.99-2.11 (m, 6H). |
| 296 | N-(4-((2-(1,1-difluoroethyl)-6-(5-methoxypyridin-3-yl)pyrimidin-4-yl)amino)-5- |
| | methoxypyridin-2-yl)acetamide |
| | 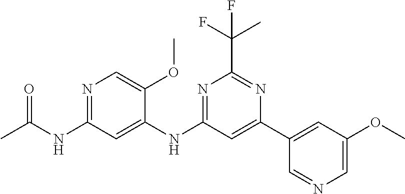 [SMILES: COc1cncc(-c2cc(Nc3cc(NC(C)=O)ncc3OC)nc(C(C)(F)F)n2)c1] |
| | LCMS m/z = 431 [M + H]⁺. |
| 297 | N-(4-((6-((2-oxaspiro[3.3]heptan-6-yl)oxy)-2-(1,1-difluoroethyl)pyrimidin-4-yl)amino)-5- |
| | ethoxypyridin-2-yl)acetamide |
| | 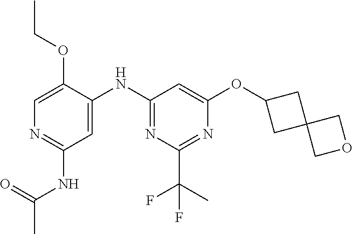 [SMILES: CCOc1cnc(NC(C)=O)cc1Nc1cc(OC2CC3(COC3)C2)nc(C(C)(F)F)n1] |
| | LCMS m/z = 450.2 [M + H]⁺. |
| 298 | N-(4-((6-cyclopropoxy-2-(1,1-difluoroethyl)pyrimidin-4-yl)amino)-5-(2- |
| | methoxyethoxy)pyridin-2-yl)acetamide |
| | 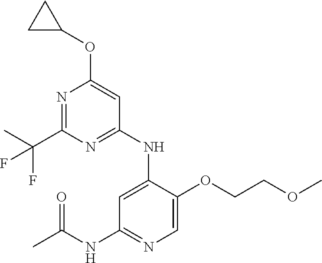 [SMILES: COCCOc1cnc(NC(C)=O)cc1Nc1cc(OC2CC2)nc(C(C)(F)F)n1] |
| | LCMS m/z = 424.1 [M + H]⁺. |
| 299 | (S)-N-(4-((6-cyclopropoxy-2-(1,1-difluoroethyl)pyrimidin-4-yl)amino)-5-(2- |
| | methoxypropoxy)pyridin-2-yl)acetamide |
| | 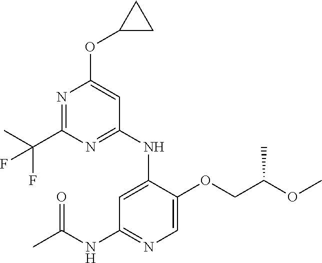 [SMILES: CO[C@@H](C)COc1cnc(NC(C)=O)cc1Nc1cc(OC2CC2)nc(C(C)(F)F)n1] |
| | LCMS m/z = 438. [M + H]⁺. |
| 300 | N-(4-((2-(1,1-difluoroethyl)-6-isopropylpyrimidin-4-yl)amino)-5-(2-methoxyethoxy)pyridin- |
| | 2-yl)acetamide |
| | 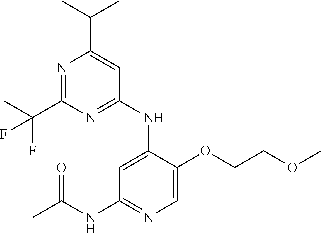 [SMILES: COCCOc1cnc(NC(C)=O)cc1Nc1cc(C(C)C)nc(C(C)(F)F)n1] |
| | LCMS m/z = 410.2 [M + H]⁺. |
| 301 | N-(4-((6-cyclopropoxy-2-(1,1-difluoroethyl)pyrimidin-4-yl)amino)-5-((1-methyl-1H- |
| | pyrazol-4-yl)oxy)pyridin-2-yl)acetamide |
| | 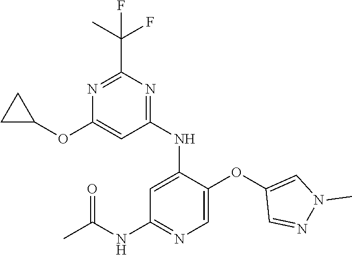 [SMILES: CC(=O)Nc1cc(Nc2cc(OC3CC3)nc(C(C)(F)F)n2)c(Oc2cnn(C)c2)cn1] |
| | LCMS m/z = 446.2 [M + H]⁺. |
| 302 | N-(4-((2-(1,1-difluoroethyl)-6-(1-methyl-1H-pyrazol-4-yl)pyrimidin-4-yl)amino)-5- |
| | ethoxypyridin-2-yl)acetamide |
| | 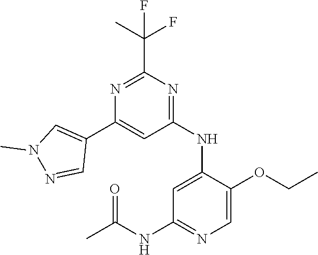 [SMILES: CCOc1cnc(NC(C)=O)cc1Nc1cc(-c2cnn(C)c2)nc(C(C)(F)F)n1] |
| | LCMS m/z = 418.2 [M + H]⁺. |
| 303 | N-(4-((2-(1,1-difluoroethyl)-6-(1-(difluoromethyl)-1H-pyrazol-4-yl)pyrimidin-4-yl)amino)- |
| | 5-ethoxypyridin-2-yl)acetamide |
| | 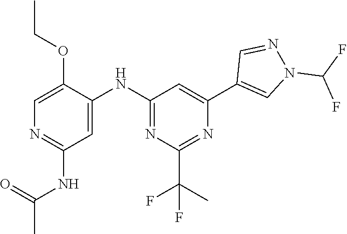 [SMILES: CCOc1cnc(NC(C)=O)cc1Nc1cc(-c2cnn(C(F)F)c2)nc(C(C)(F)F)n1] |
| | LCMS m/z = 454.2 [M + H]⁺. |
| 304 | N-(4-((6-(1-cyclopropyl-1H-pyrazol-4-yl)-2-(1,1-difluoroethyl)pyrimidin-4-yl)amino)-5- |
| | ethoxypyridin-2-yl)acetamide |
| | 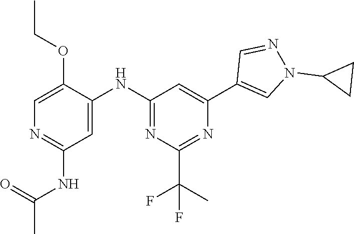 [SMILES: CCOc1cnc(NC(C)=O)cc1Nc1cc(-c2cnn(C3CC3)c2)nc(C(C)(F)F)n1] |
| | LCMS m/z = 444.2 [M + H]⁺. |
| 305 | 2-(4-(6-((2-acetamido-5-ethoxypyridin-4-yl)amino)-2-(1,1-difluoroethyl)pyrimidin-4-yl)- |
| | 1H-pyrazol-1-yl)acetic acid |
| | 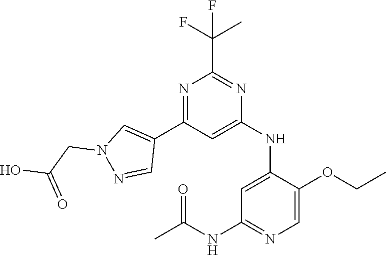 [SMILES: CCOc1cnc(NC(C)=O)cc1Nc1cc(-c2cnn(CC(=O)O)c2)nc(C(C)(F)F)n1] |
| | LCMS m/z = 462.2 [M + H]⁺. |
| 306 | N-(4-((2-(1,1-difluoroethyl)-6-(1-isopropyl-1H-pyrazol-4-yl)pyrimidin-4-yl)amino)-5- |
| | ethoxypyridin-2-yl)acetamide |
| | 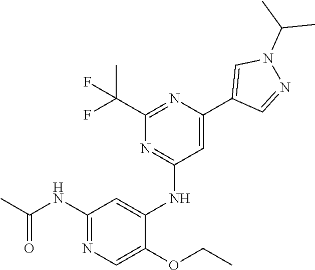 [SMILES: CCOc1cnc(NC(C)=O)cc1Nc1cc(-c2cnn(C(C)C)c2)nc(C(C)(F)F)n1] |
| | LCMS m/z = 446.2 [M + H]⁺. |
| 307 | tert-butyl 4-(6-((2-acetamido-5-ethoxypyridin-4-yl)amino)-2-(1,1-difluoroethyl)pyrimidin-4- |
| | yl)-1H-pyrazole-1-carboxylate |
| | 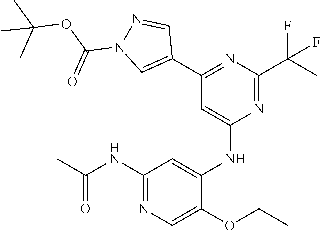 [SMILES: CCOc1cnc(NC(C)=O)cc1Nc1cc(-c2cnn(C(=O)OC(C)(C)C)c2)nc(C(C)(F)F)n1] |
| | LCMS m/z = 504.2 [M + H]⁺. |
| 308 | N-(4-((2-(1,1-difluoroethyl)-6-(1-(2-(dimethylamino)ethyl)-1H-pyrazol-4-yl)pyrimidin-4- |
| | yl)amino)-5-ethoxypyridin-2-yl)acetamide |
| | 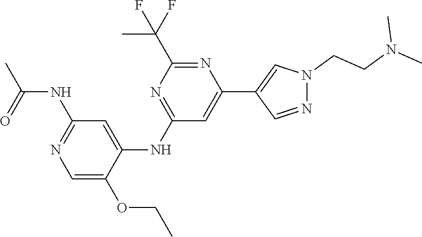 [SMILES: CCOc1cnc(NC(C)=O)cc1Nc1cc(-c2cnn(CCN(C)C)c2)nc(C(C)(F)F)n1] |
| | LCMS m/z = 475.2 [M + H]⁺. |
| 309 | N-(4-((2-(1,1-difluoroethyl)-6-(1-(2,2,2-trifluoroethyl)-1H-pyrazol-4-yl)pyrimidin-4- |
| | yl)amino)-5-ethoxypyridin-2-yl)acetamide |
| | 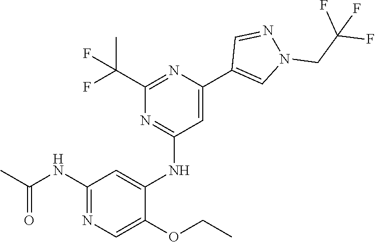 [SMILES: CCOc1cnc(NC(C)=O)cc1Nc1cc(-c2cnn(CC(F)(F)F)c2)nc(C(C)(F)F)n1] |
| | LCMS m/z = 486.2 [M + H]⁺. |
| 310 | N-(4-((2-(1,1-difluoroethyl)-6-(1-(2-methoxyethyl)-1H-pyrazol-4-yl)pyrimidin-4-yl)amino)- |
| | 5-ethoxypyridin-2-yl)acetamide |
| | 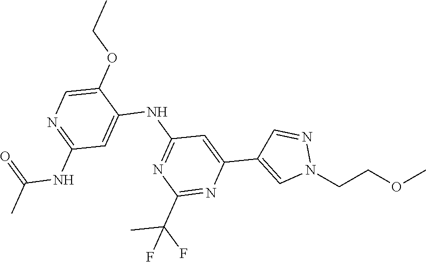 [SMILES: CCOc1cnc(NC(C)=O)cc1Nc1cc(-c2cnn(CCOC)c2)nc(C(C)(F)F)n1] |
| | LCMS m/z = 462.2 [M + H]⁺. |
| 311 | N-(4-((2-(1,1-difluoroethyl)-6-(1-ethyl-1H-pyrazol-4-yl)pyrimidin-4-yl)amino)-5- |
| | ethoxypyridin-2-yl)acetamide |
| | 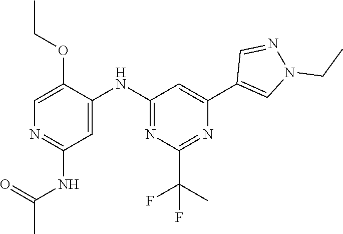 [SMILES: CCOc1cnc(NC(C)=O)cc1Nc1cc(-c2cnn(CC)c2)nc(C(C)(F)F)n1] |
| | LCMS m/z = 432.2 [M + H]⁺. |
| 312 | N-(4-((6-(1-cyclobutyl-1H-pyrazol-4-yl)-2-(1,1-difluoroethyl)pyrimidin-4-yl)amino)-5- |
| | ethoxypyridin-2-yl)acetamide |
| | 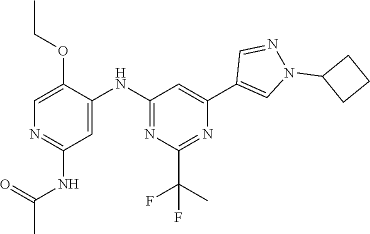 [SMILES: CCOc1cnc(NC(C)=O)cc1Nc1cc(-c2cnn(C3CCC3)c2)nc(C(C)(F)F)n1] |
| | LCMS m/z = 458.2 [M + H]⁺. |
| 313 | N-(4-((2-(1,1-difluoroethyl)-6-(1-(2-morpholinoethyl)-1H-pyrazol-4-yl)pyrimidin-4- |
| | yl)amino)-5-ethoxypyridin-2-yl)acetamide |
| | 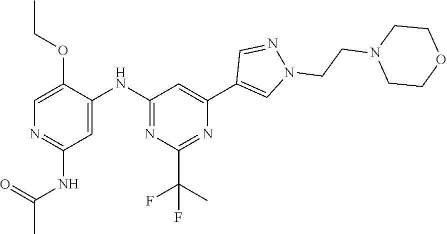 [SMILES: CCOc1cnc(NC(C)=O)cc1Nc1cc(-c2cnn(CCN3CCOCC3)c2)nc(C(C)(F)F)n1] |
| | LCMS m/z = 517.2 [M + H]⁺. |
| 314 | N-(4-((2-(1,1-difluoroethyl)-6-(1-(methyl-d3)-1H-pyrazol-4-yl)pyrimidin-4-yl)amino)-5- |
| | ethoxypyridin-2-yl)acetamide |
| | 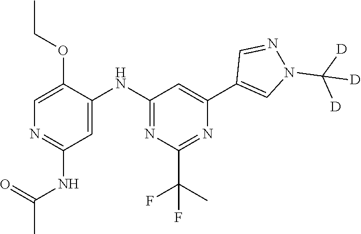 [SMILES: [2H]C([2H])([2H])n1cc(-c2cc(Nc3cc(NC(C)=O)ncc3OCC)nc(C(C)(F)F)n2)cn1] |
| | LCMS m/z = 421.2 [M + H]⁺. |
| 315 | N-(4-((2-(1,1-difluoroethyl)-6-isopropylpyrimidin-4-yl)amino)-5-(methoxy-d3)pyridin-2- |
| | yl)acetamide |
| | 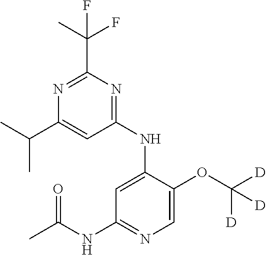 [SMILES: [2H]C([2H])([2H])Oc1cnc(NC(C)=O)cc1Nc1cc(C(C)C)nc(C(C)(F)F)n1] |
| | LCMS m/z = 369.2 [M + H]⁺. |
| 316 | N-(5-(2-cyclopropoxyethoxy)-4-((2-(1,1-difluoroethyl)-6-methylpyrimidin-4- |
| | yl)amino)pyridin-2-yl)acetamide |
| | 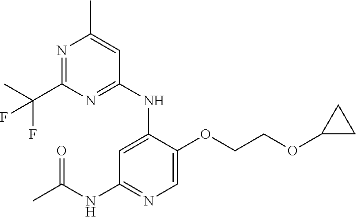 [SMILES: CC(=O)Nc1cc(Nc2cc(C)nc(C(C)(F)F)n2)c(OCCOC2CC2)cn1] |
| | LCMS m/z = 408 [M + H]⁺. |
| 317 | N-(4-((2-(1,1-difluoroethyl)-6-methylpyrimidin-4-yl)amino)-5-((4-methyltetrahydro-2H- |
| | pyran-4-yl)methoxy)pyridin-2-yl)acetamide |
| | 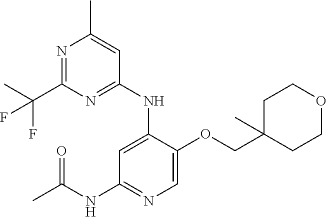 [SMILES: CC(=O)Nc1cc(Nc2cc(C)nc(C(C)(F)F)n2)c(OCC2(C)CCOCC2)cn1] |
| | LCMS m/z = 436 [M + H]⁺. |
| 318 | (S)-N-(5-(2-(tert-butoxy)propoxy)-4-((2-(1,1-difluoroethyl)-6-methylpyrimidin-4- |
| | yl)amino)pyridin-2-yl)acetamide |
| | 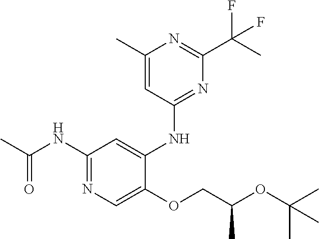 [SMILES: CC(=O)Nc1cc(Nc2cc(C)nc(C(C)(F)F)n2)c(OC[C@H](C)OC(C)(C)C)cn1] |
| | LCMS m/z = 438 [M + H]⁺. |
| 319 | N-(4-((2-(1,1-difluoroethyl)-6-methylpyrimidin-4-yl)amino)-5-((3-ethylisoxazol-5- |
| | yl)methoxy)pyridin-2-yl)acetamide |
| | 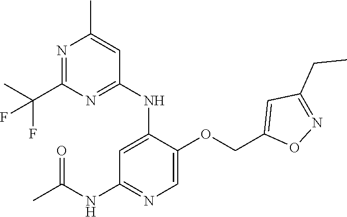 [SMILES: CCc1cc(COc2cnc(NC(C)=O)cc2Nc2cc(C)nc(C(C)(F)F)n2)on1] |
| | LCMS m/z = 433 [M + H]⁺. |
| 320 | N-(4-((2-(1,1-difluoroethyl)-6-methylpyrimidin-4-yl)amino)-5-((2-methyloxazol-5- |
| | yl)methoxy)pyridin-2-yl)acetamide |
| | 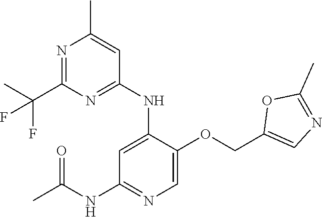 [SMILES: CC(=O)Nc1cc(Nc2cc(C)nc(C(C)(F)F)n2)c(OCc2cnc(C)o2)cn1] |
| | LCMS m/z = 419 [M + H]⁺. |
| 321 | (R)-N-(5-(2-(tert-butoxy)propoxy)-4-((2-(1,1-difluoroethyl)-6-methylpyrimidin-4- |
| | yl)amino)pyridin-2-yl)acetamide |
| | 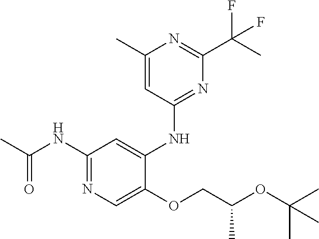 [SMILES: CC(=O)Nc1cc(Nc2cc(C)nc(C(C)(F)F)n2)c(OC[C@@H](C)OC(C)(C)C)cn1] |
| | LCMS m/z = 438 [M + H]⁺. |
| 322 | N-(4-((2-(1,1-difluoroethyl)-6-methylpyrimidin-4-yl)amino)-5-((3-methoxyisoxazol-5- |
| | yl)methoxy)pyridin-2-yl)acetamide |
| | 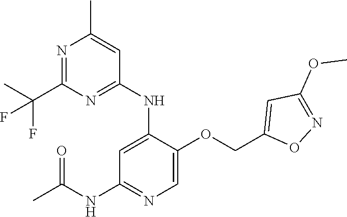 [SMILES: COc1cc(COc2cnc(NC(C)=O)cc2Nc2cc(C)nc(C(C)(F)F)n2)on1] |
| | LCMS m/z = 435 [M + H]⁺. |
| 323 | N-(4-((2-(1,1-difluoroethyl)-6-methylpyrimidin-4-yl)amino)-5-(isothiazol-3- |
| | ylmethoxy)pyridin-2-yl)acetamide |
| | 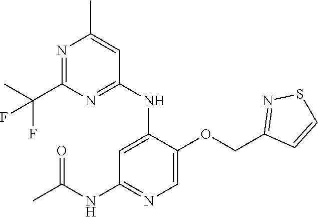 [SMILES: CC(=O)Nc1cc(Nc2cc(C)nc(C(C)(F)F)n2)c(OCc2ccsn2)cn1] |
| | LCMS m/z = 421 [M + H]⁺. |
| 324 | N-(5-((3-oxabicyclo[3.1.1]heptan-1-yl)methoxy)-4-((2-(1,1-difluoroethyl)-6- |
| | methylpyrimidin-4-yl)amino)pyridin-2-yl)acetamide |
| | 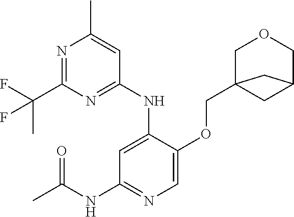 [SMILES: CC(=O)Nc1cc(Nc2cc(C)nc(C(C)(F)F)n2)c(OCC23COCC(C2)C3)cn1] |
| | LCMS m/z = 434 [M + H]⁺. |
| 325 | N-(4-((2-(1,1-difluoroethyl)-6-methylpyrimidin-4-yl)amino)-5-((4-methyloxazol-2- |
| | yl)methoxy)pyridin-2-yl)acetamide |
| | 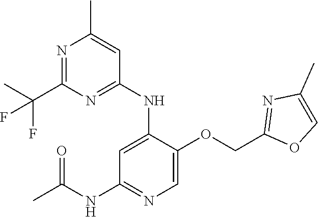 [SMILES: CC(=O)Nc1cc(Nc2cc(C)nc(C(C)(F)F)n2)c(OCc2nc(C)co2)cn1] |
| | LCMS m/z = 419 [M + H]⁺. |
| 326 | N-(4-((2-(1,1-difluoroethyl)-6-methylpyrimidin-4-yl)amino)-5-((1- |
| | (methoxymethyl)cyclopropyl)methoxy)pyridin-2-yl)acetamide |
| | 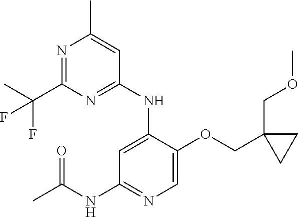 [SMILES: COCC1(COc2cnc(NC(C)=O)cc2Nc2cc(C)nc(C(C)(F)F)n2)CC1] |
| | LCMS m/z = 422 [M + H]⁺. |
| 327 | N-(4-((2-(1,1-difluoroethyl)-6-methylpyrimidin-4-yl)amino)-5-((5-ethyloxazol-2- |
| | yl)methoxy)pyridin-2-yl)acetamide |
| | 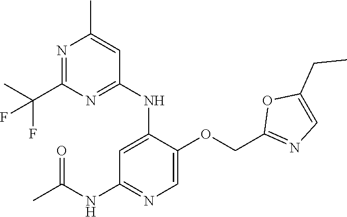 [SMILES: CCc1cnc(COc2cnc(NC(C)=O)cc2Nc2cc(C)nc(C(C)(F)F)n2)o1] |
| | LCMS m/z = 433 [M + H]⁺. |
| 328 | N-(4-((2-(1,1-difluoroethyl)-6-methylpyrimidin-4-yl)amino)-5-(2-(2-fluoropyridin-3- |
| | yl)ethoxy)pyridin-2-yl)acetamide |
| | 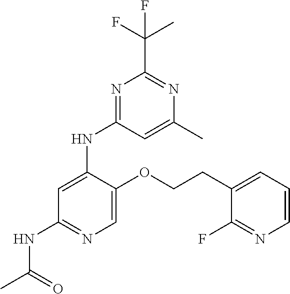 [SMILES: CC(=O)Nc1cc(Nc2cc(C)nc(C(C)(F)F)n2)c(OCCc2cccnc2F)cn1] |
| | LCMS m/z = 447 [M + H]⁺. |
| 329 | N-(4-((2-(1,1-difluoroethyl)-6-methylpyrimidin-4-yl)amino)-5-((5-methylthiazol-4- |
| | yl)methoxy)pyridin-2-yl)acetamide |
| | 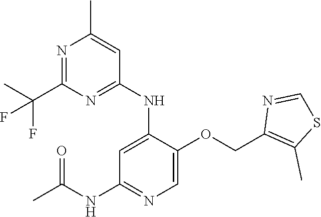 [SMILES: CC(=O)Nc1cc(Nc2cc(C)nc(C(C)(F)F)n2)c(OCc2ncsc2C)cn1] |
| | LCMS m/z = 435 [M + H]⁺. |
| 330 | N-(4-((2-(1,1-difluoroethyl)-6-methylpyrimidin-4-yl)amino)-5-((4,6-dimethylpyrimidin-5- |
| | yl)methoxy)pyridin-2-yl)acetamide |
| | 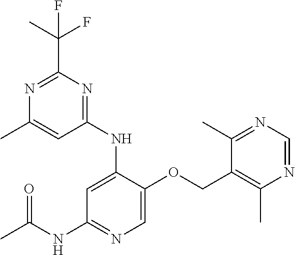 [SMILES: CC(=O)Nc1cc(Nc2cc(C)nc(C(C)(F)F)n2)c(OCc2c(C)ncnc2C)cn1] |
| | LCMS m/z = 444 [M + H]⁺. |
| 331 | N-(4-((2-(1,1-difluoroethyl)-6-methylpyrimidin-4-yl)amino)-5-((3-ethyl-1,2,4-oxadiazol-5- |
| | yl)methoxy)pyridin-2-yl)acetamide |
| | 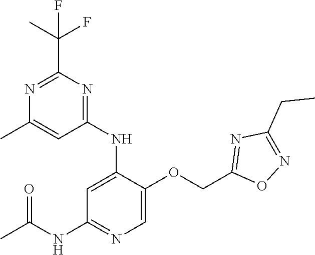 [SMILES: CCc1noc(COc2cnc(NC(C)=O)cc2Nc2cc(C)nc(C(C)(F)F)n2)n1] |
| | LCMS m/z = 434 [M + H]⁺. |
| 332 | N-(4-((2-(1,1-difluoroethyl)-6-methylpyrimidin-4-yl)amino)-5-(((1r,3r)-3- |
| | fluorocyclobutyl)methoxy)pyridin-2-yl)acetamide |
| | 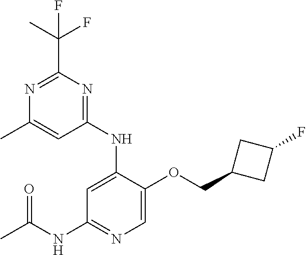 [SMILES: CC(=O)Nc1cc(Nc2cc(C)nc(C(C)(F)F)n2)c(OC[C@H]2C[C@H](F)C2)cn1] |
| | LCMS m/z = 410 [M + H]⁺. |
| 333 | N-(5-((5-cyanofuran-2-yl)methoxy)-4-((2-(1,1-difluoroethyl)-6-methylpyrimidin-4- |
| | yl)amino)pyridin-2-yl)acetamide |
| | 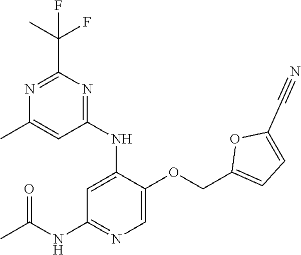 [SMILES: CC(=O)Nc1cc(Nc2cc(C)nc(C(C)(F)F)n2)c(OCc2ccc(C#N)o2)cn1] |
| | LCMS m/z = 429 [M + H]⁺. |
| 334 | N-(4-((2-(1,1-difluoroethyl)-6-methylpyrimidin-4-yl)amino)-5-((4-methylthiazol-5- |
| | yl)methoxy)pyridin-2-yl)acetamide |
| | 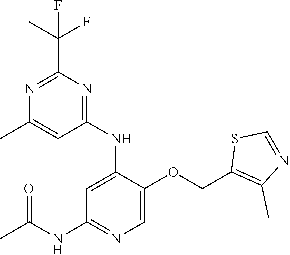 [SMILES: CC(=O)Nc1cc(Nc2cc(C)nc(C(C)(F)F)n2)c(OCc2scnc2C)cn1] |
| | LCMS m/z = 435 [M + H]⁺. |
| 335 | N-(4-((2-(1,1-difluoroethyl)-6-methylpyrimidin-4-yl)amino)-5-((3-methylisoxazol-4- |
| | yl)methoxy)pyridin-2-yl)acetamide |
| | 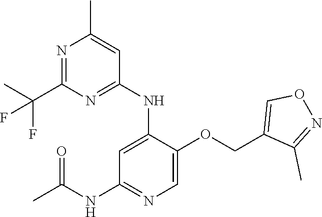 [SMILES: CC(=O)Nc1cc(Nc2cc(C)nc(C(C)(F)F)n2)c(OCc2conc2C)cn1] |
| | LCMS m/z = 419 [M + H]⁺. |
| 336 | (R)-N-(4-((2-(1,1-difluoroethyl)-6-methylpyrimidin-4-yl)amino)-5-(3-fluorobutoxy)pyridin- |
| | 2-yl)acetamide |
| | 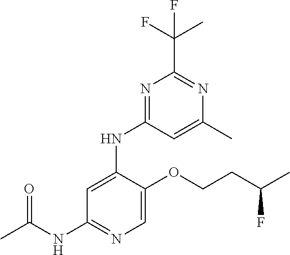 [SMILES: CC(=O)Nc1cc(Nc2cc(C)nc(C(C)(F)F)n2)c(OCC[C@@H](C)F)cn1] |
| | LCMS m/z = 398 [M + H]⁺. |
| 337 | N-(4-((2-(1,1-difluoroethyl)-6-methylpyrimidin-4-yl)amino)-5-((4-methoxytetrahydro-2H- |
| | pyran-4-yl)methoxy)pyridin-2-yl)acetamide |
| | 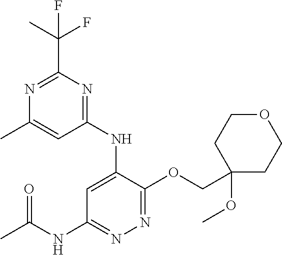 [SMILES: COC1(COc2nnc(NC(C)=O)cc2Nc2cc(C)nc(C(C)(F)F)n2)CCOCC1] |
| | LCMS m/z = 452 [M + H]⁺. |
| 338 | N-(4-((2-(1,1-difluoroethyl)-6-methylpyrimidin-4-yl)amino)-5-((4,5-dimethyloxazol-2- |
| | yl)methoxy)pyridin-2-yl)acetamide |
| | 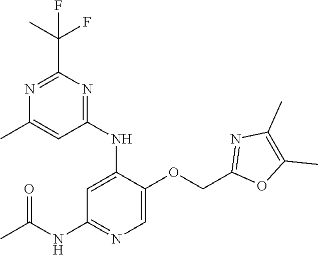 |
| | LCMS m/z = 433 [M + H]⁺. |
| 339 | (R)-N-(4-((2-(1,1-difluoroethyl)-6-methylpyrimidin-4-yl)amino)-5-(3-fluorobutoxy)pyridin- |
| | 2-yl)acetamide |
| | 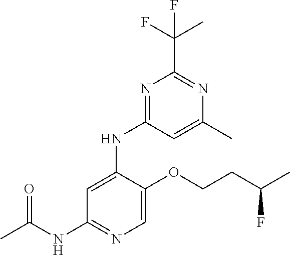 [SMILES: CC(=O)Nc1cc(Nc2cc(C)nc(C(C)(F)F)n2)c(OCC[C@@H](C)F)cn1] |
| | LCMS m/z = 398 [M + H]⁺. |
| 340 | N-(4-((2-(1,1-difluoroethyl)-6-methylpyrimidin-4-yl)amino)-5-((4-(fluoromethyl)tetrahydro- |
| | 2H-pyran-4-yl)methoxy)pyridin-2-yl)acetamide |
| | 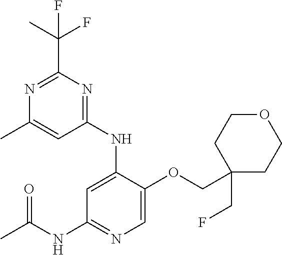 [SMILES: CC(=O)Nc1cc(Nc2cc(C)nc(C(C)(F)F)n2)c(OCC2(CF)CCOCC2)cn1] |
| | LCMS m/z = 454 [M + H]⁺. |
| 341 | N-(4-((2-(1,1-difluoroethyl)-6-methylpyrimidin-4-yl)amino)-5-((5-methyloxazol-2- |
| | yl)methoxy)pyridin-2-yl)acetamide |
| | 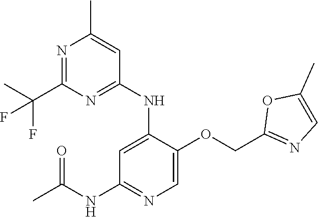 [SMILES: CC(=O)Nc1cc(Nc2cc(C)nc(C(C)(F)F)n2)c(OCc2ncc(C)o2)cn1] |
| | LCMS m/z = 419 [M + H]⁺. |
| 342 | N-(5-((5-cyclopropyloxazol-2-yl)methoxy)-4-((2-(1,1-difluoroethyl)-6-methylpyrimidin-4- |
| | yl)amino)pyridin-2-yl)acetamide |
| | 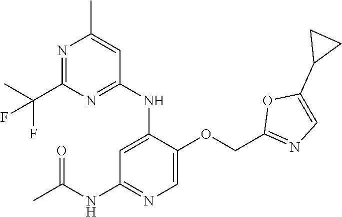 [SMILES: CC(=O)Nc1cc(Nc2cc(C)nc(C(C)(F)F)n2)c(OCc2ncc(C3CC3)o2)cn1] |
| | LCMS m/z = 445 [M + H]⁺. |
| 343 | N-(4-((2-(1,1-difluoroethyl)-6-methylpyrimidin-4-yl)amino)-5-((3-methyl-1,2,4-thiadiazol- |
| | 5-yl)methoxy)pyridin-2-yl)acetamide |
| | 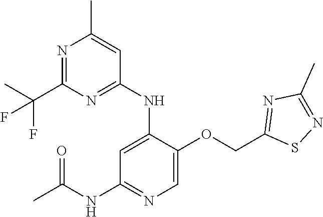 [SMILES: CC(=O)Nc1cc(Nc2cc(C)nc(C(C)(F)F)n2)c(OCc2nc(C)ns2)cn1] |
| | LCMS m/z = 436 [M + H]⁺. |
| 344 | N-(5-((2-cyclopropyloxazol-5-yl)methoxy)-4-((2-(1,1-difluoroethyl)-6-methylpyrimidin-4- |
| | yl)amino)pyridin-2-yl)acetamide |
| | 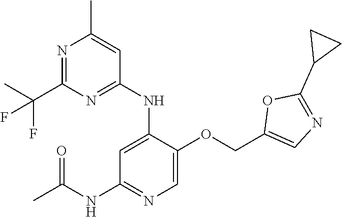 [SMILES: CC(=O)Nc1cc(Nc2cc(C)nc(C(C)(F)F)n2)c(OCc2cnc(C3CC3)o2)cn1] |
| | LCMS m/z = 445 [M + H]⁺. |
| 345 | N-(4-((2-(1,1-difluoroethyl)-6-methylpyrimidin-4-yl)amino)-5-((4-methoxypyrimidin-2- |
| | yl)methoxy)pyridin-2-yl)acetamide |
| | 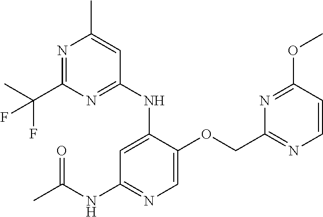 [SMILES: COc1ccnc(COc2cnc(NC(C)=O)cc2Nc2cc(C)nc(C(C)(F)F)n2)n1] |
| | LCMS m/z = 446 [M + H]⁺. |
| 346 | N-(4-((2-(1,1-difluoroethyl)-6-methylpyrimidin-4-yl)amino)-5-((6-methoxypyrimidin-4- |
| | yl)methoxy)pyridin-2-yl)acetamide |
| | 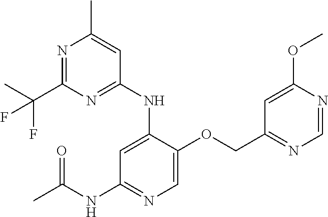 [SMILES: COc1cc(COc2cnc(NC(C)=O)cc2Nc2cc(C)nc(C(C)(F)F)n2)ncn1] |
| | LCMS m/z = 446 [M + H]⁺. |
| 347 | N-(4-((2-(1,1-difluoroethyl)-6-methylpyrimidin-4-yl)amino)-5-(((1s,4s)-4-methyl-2- |
| | oxabicyclo[2.2.2]octan-1-yl)methoxy)pyridin-2-yl)acetamide |
| | 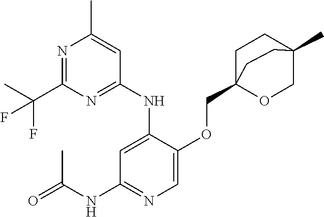 [SMILES: CC(=O)Nc1cc(Nc2cc(C)nc(C(C)(F)F)n2)c(OC[C@]23CC[C@](C)(CC2)CO3)cn1] |
| | LCMS m/z = 462 [M + H]⁺. |
| 348 | N-(4-((6-cyclopropoxy-2-(1,1-difluoroethyl)pyrimidin-4-yl)amino)-5-(pyridin-2- |
| | ylmethoxy)pyridin-2-yl)acetamide |
| | 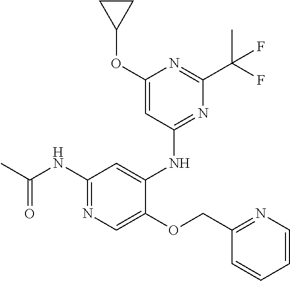 [SMILES: CC(=O)Nc1cc(Nc2cc(OC3CC3)nc(C(C)(F)F)n2)c(OCc2ccccn2)cn1] |
| | LCMS m/z = 457 [M + H]⁺. |
| 349 | N-(4-((6-cyclopropoxy-2-(1,1-difluoroethyl)pyrimidin-4-yl)amino)-5-((3,4- |
| | difluorobenzyl)oxy)pyridin-2-yl)acetamide |
| | 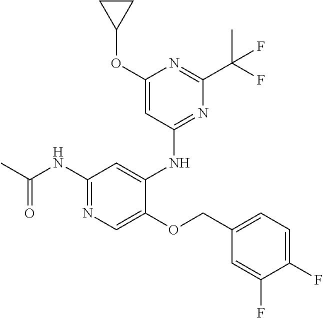 [SMILES: CC(=O)Nc1cc(Nc2cc(OC3CC3)nc(C(C)(F)F)n2)c(OCc2ccc(F)c(F)c2)cn1] |
| | LCMS m/z = 492 [M + H]⁺. |
| 350 | N-(4-((6-cyclopropoxy-2-(1,1-difluoroethyl)pyrimidin-4-yl)amino)-5-((1r,3r)-3- |
| | methoxycyclobutoxy)pyridin-2-yl)acetamide |
| | 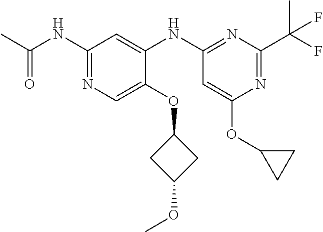 [SMILES: CO[C@H]1C[C@H](Oc2cnc(NC(C)=O)cc2Nc2cc(OC3CC3)nc(C(C)(F)F)n2)C1] |
| | LCMS m/z = 450 [M + H]⁺. |
| 351 | N-(4-((6-cyclopropoxy-2-(1,1-difluoroethyl)pyrimidin-4-yl)amino)-5-((1s,3s)-3- |
| | methoxycyclobutoxy)pyridin-2-yl)acetamide |
| | 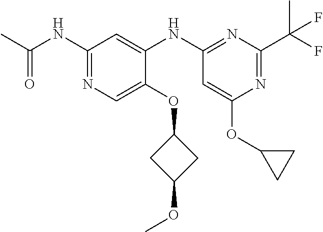 [SMILES: CO[C@H]1C[C@@H](Oc2cnc(NC(C)=O)cc2Nc2cc(OC3CC3)nc(C(C)(F)F)n2)C1] |
| | LCMS m/z = 450 [M + H]⁺. |
| 352 | N-(4-((6-cyclopropoxy-2-(1,1-difluoroethyl)pyrimidin-4-yl)amino)-5-((1r,3r)-3- |
| | fluorocyclobutoxy)pyridin-2-yl)acetamide |
| | 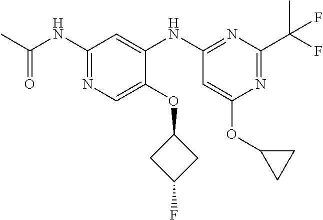 [SMILES: CC(=O)Nc1cc(Nc2cc(OC3CC3)nc(C(C)(F)F)n2)c(O[C@H]2C[C@H](F)C2)cn1] |
| | LCMS m/z = 438 [M + H]⁺. |
| 353 | N-(4-((6-cyclopropoxy-2-(1,1-difluoroethyl)pyrimidin-4-yl)amino)-5-((1s,3s)-3- |
| | fluorocyclobutoxy)pyridin-2-yl)acetamide |
| | 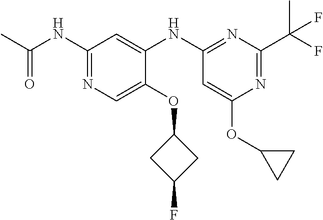 [SMILES: CC(=O)Nc1cc(Nc2cc(OC3CC3)nc(C(C)(F)F)n2)c(O[C@H]2C[C@@H](F)C2)cn1] |
| | LCMS m/z = 438 [M + H]⁺. |
| 354 | N-(5-((1-cyclobutyl-1H-pyrazol-4-yl)methoxy)-4-((2-(1,1-difluoroethyl)-6-methylpyrimidin- |
| | 4-yl)amino)pyridin-2-yl)acetamide |
| | 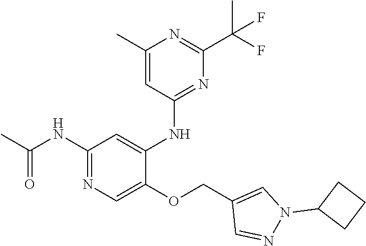 [SMILES: CC(=O)Nc1cc(Nc2cc(C)nc(C(C)(F)F)n2)c(OCc2cnn(C3CCC3)c2)cn1] |
| | LCMS m/z = 458 [M + H]⁺. |
| 355 | N-(4-((2-(1,1-difluoroethyl)-6-methylpyrimidin-4-yl)amino)-5-(pyridin-3- |
| | ylmethoxy)pyridin-2-yl)acetamide |
| | 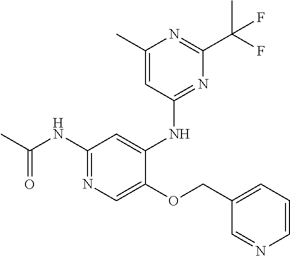 [SMILES: CC(=O)Nc1cc(Nc2cc(C)nc(C(C)(F)F)n2)c(OCc2cccnc2)cn1] |
| | LCMS m/z = 415.2 [M + H]⁺. |
| 356 | N-(4-((2-(1,1-difluoroethyl)-6-methylpyrimidin-4-yl)amino)-5-((5-methoxypyrazin-2- |
| | yl)oxy)pyridin-2-yl)acetamide |
| | 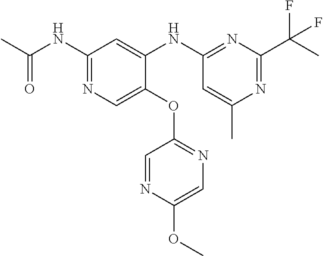 [SMILES: COc1cnc(Oc2cnc(NC(C)=O)cc2Nc2cc(C)nc(C(C)(F)F)n2)cn1] |
| | LCMS m/z = 432.2 [M + H]⁺. |
| 357 | N-(4-((2-(1,1-difluoroethyl)-6-methylpyrimidin-4-yl)amino)-5-((6-methoxypyrazin-2- |
| | yl)oxy)pyridin-2-yl)acetamide |
| | 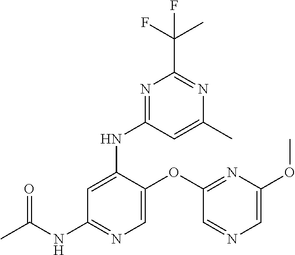 [SMILES: COc1cncc(Oc2cnc(NC(C)=O)cc2Nc2cc(C)nc(C(C)(F)F)n2)n1] |
| | LCMS m/z = 432.2 [M + H]⁺. |
| 358 | N-(4-((2-(1,1-difluoroethyl)-6-methylpyrimidin-4-yl)amino)-5-((6-methoxypyridazin-3- |
| | yl)oxy)pyridin-2-yl)acetamide |
| | 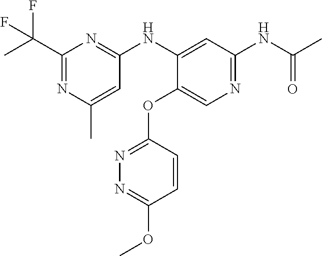 [SMILES: COc1ccc(Oc2cnc(NC(C)=O)cc2Nc2cc(C)nc(C(C)(F)F)n2)nn1] |
| | LCMS m/z = 432.2 [M + H]⁺. |
| 359 | N-(4-((2-(1,1-difluoroethyl)-6-methylpyrimidin-4-yl)amino)-5-((2- |
| | (trifluoromethyl)pyrimidin-4-yl)oxy)pyridin-2-yl)acetamide |
| | 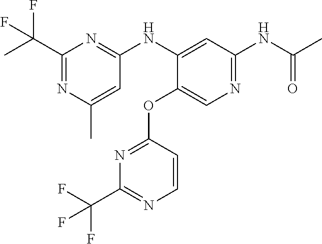 [SMILES: CC(=O)Nc1cc(Nc2cc(C)nc(C(C)(F)F)n2)c(Oc2ccnc(C(F)(F)F)n2)cn1] |
| | LCMS m/z = 470.1 [M + H]⁺. |
| 360 | methyl (4-((2-(1,1-difluoroethyl)-6-methylpyrimidin-4-yl)amino)-5-ethoxypyridin-2- |
| | yl)carbamate |
| | 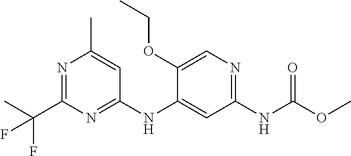 |
| | LCMS m/z = 368.1 [M + H]⁺; ¹H NMR (600 MHz, DMSO-d₆) δ ppm 9.83 (s, 1H), 9.11 (s, |
| | 1H), 8.74 (s, 1H), 7.96 (s, 1H), 7.15 (s, 1H), 4.17 (q, J = 7.12 Hz, 2H), 3.64 (s, 3H), 2.51- |
| | 2.57 (m, 1H), 2.33-2.47 (m, 3H), 2.05 (t, J = 19.07 Hz, 3H), 1.37 (t, J = 6.87 Hz, 2H) |
| 361 | N-(4-((2-(1,1-difluoroethyl)-6-(1-isopropyl-1H-pyrazol-4-yl)pyrimidin-4-yl)amino)-5- |
| | (ethoxy-d5)pyridin-2-yl)acetamide |
| | 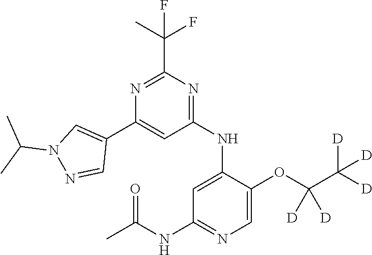 [SMILES: [2H]C([2H])([2H])C([2H])([2H])Oc1cnc(NC(C)=O)cc1Nc1cc(-c2cnn(C(C)C)c2)nc(C(C)(F)F)n1] |
| | LCMS m/z = 451.2 [M + H]⁺. |
| 362 | N-(4-((2-(1,1-difluoroethyl)-6-isopropylpyrimidin-4-yl)amino)-5-(ethoxy-d5)pyridin-2- |
| | yl)acetamide |
| | 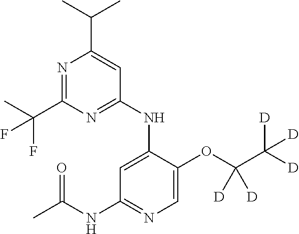 [SMILES: [2H]C([2H])([2H])C([2H])([2H])Oc1cnc(NC(C)=O)cc1Nc1cc(C(C)C)nc(C(C)(F)F)n1] |
| | LCMS m/z = 385.2 [M + H]⁺. |
| 363 | N-(4-((2-(1,1-difluoroethyl)-6-(1-isopropyl-1H-pyrazol-4-yl)pyrimidin-4-yl)amino)-5- |
| | methoxypyridin-2-yl)acetamide |
| | 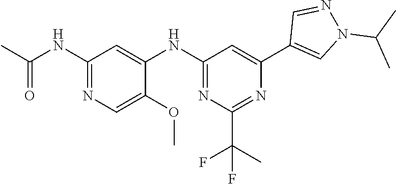 [SMILES: COc1cnc(NC(C)=O)cc1Nc1cc(-c2cnn(C(C)C)c2)nc(C(C)(F)F)n1] |
| | LCMS m/z = 432 [M + H]⁺ |
| 364 | N-(4-((2-(1,1-difluoroethyl)-6-isopropylpyrimidin-4-yl)amino)-5-(ethoxy-1,1-d2)pyridin-2- |
| | yl)acetamide |
| | 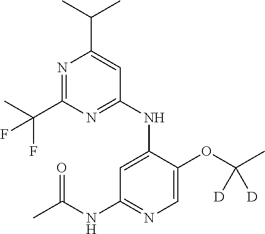 [SMILES: [2H]C([2H])(C)Oc1cnc(NC(C)=O)cc1Nc1cc(C(C)C)nc(C(C)(F)F)n1] |
| | LCMS m/z = 382.2 [M + H]⁺. |
| 365 | N-(4-((2-(1,1-difluoroethyl)-6-isopropylpyrimidin-4-yl)amino)-5-(pyridin-2- |
| | ylmethoxy)pyridin-2-yl)acetamide |
| | 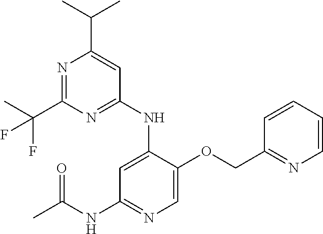 [SMILES: CC(=O)Nc1cc(Nc2cc(C(C)C)nc(C(C)(F)F)n2)c(OCc2ccccn2)cn1] |
| | LCMS m/z = 443.2 [M + H]⁺. |
| 366 | N-(4-((2-(1,1-difluoroethyl)-6-(1-isopropyl-1H-pyrazol-4-yl)pyrimidin-4-yl)amino)-5-(2- |
| | methoxyethoxy)pyridin-2-yl)acetamide |
| | 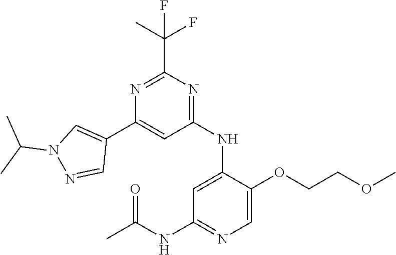 [SMILES: COCCOc1cnc(NC(C)=O)cc1Nc1cc(-c2cnn(C(C)C)c2)nc(C(C)(F)F)n1] |
| | LCMS m/z = 476.2 [M + H]⁺. |
| 367 | N-(4-((2-(1,1-difluoroethyl)-6-(5-methylpyridin-3-yl)pyrimidin-4-yl)amino)-5- |
| | ethoxypyridin-2-yl)acetamide |
| | 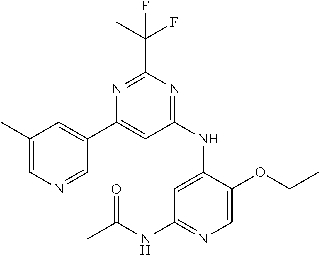 [SMILES: CCOc1cnc(NC(C)=O)cc1Nc1cc(-c2cncc(C)c2)nc(C(C)(F)F)n1] |
| | LCMS m/z = 429.2 [M + H]⁺. |
| 368 | N-(4-((2-(1,1-difluoroethyl)-6-(6-ethylpyridin-3-yl)pyrimidin-4-yl)amino)-5-ethoxypyridin- |
| | 2-yl)acetamide |
| | 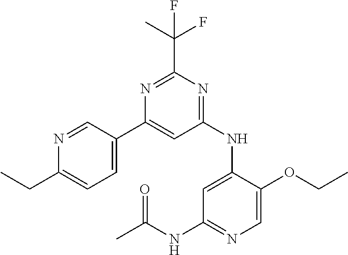 [SMILES: CCOc1cnc(NC(C)=O)cc1Nc1cc(-c2ccc(CC)nc2)nc(C(C)(F)F)n1] |
| | LCMS m/z = 443.2 [M + H]⁺. |
| 369 | N-(4-((2-(1,1-difluoroethyl)-6-(6-methoxypyridin-3-yl)pyrimidin-4-yl)amino)-5- |
| | ethoxypyridin-2-yl)acetamide |
| | 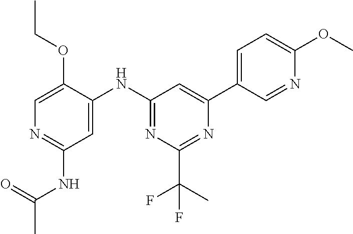 [SMILES: CCOc1cnc(NC(C)=O)cc1Nc1cc(-c2ccc(OC)nc2)nc(C(C)(F)F)n1] |
| | LCMS m/z = 445.2 [M + H]⁺. |
| 370 | N-(4-((2-(1,1-difluoroethyl)-6-(5-methoxypyridin-3-yl)pyrimidin-4-yl)amino)-5- |
| | ethoxypyridin-2-yl)acetamide |
| | 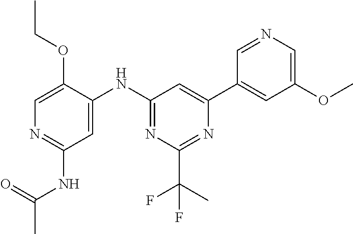 [SMILES: CCOc1cnc(NC(C)=O)cc1Nc1cc(-c2cncc(OC)c2)nc(C(C)(F)F)n1] |
| | LCMS m/z = 445.2 [M + H]⁺. |
| 371 | N-(4-((2-(1,1-difluoroethyl)-6-(1-isobutyl-1H-pyrazol-4-yl)pyrimidin-4-yl)amino)-5- |
| | ethoxypyridin-2-yl)acetamide |
| | 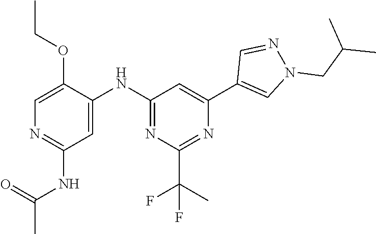 [SMILES: CCOc1cnc(NC(C)=O)cc1Nc1cc(-c2cnn(CC(C)C)c2)nc(C(C)(F)F)n1] |
| | LCMS m/z = 460.2 [M + H]⁺. |
| 372 | N-(4-((6-chloro-2-(1,1-difluoroethyl)pyrimidin-4-yl)amino)-5-ethoxypyridin-2-yl)acetamide |
| | 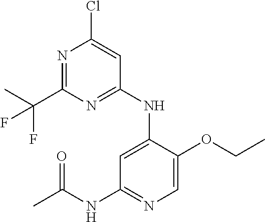 [SMILES: CCOc1cnc(NC(C)=O)cc1Nc1cc(Cl)nc(C(C)(F)F)n1] |
| | LCMS m/z = 372.1 [M + H]⁺. |
| 373 | N-(4-((2-(1,1-difluoroethyl)-6-(3-fluoropyridin-4-yl)pyrimidin-4-yl)amino)-5- |
| | methoxypyridin-2-yl)acetamide |
| | 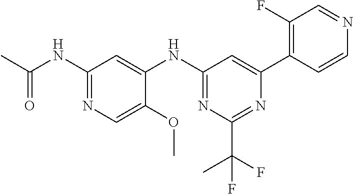 [SMILES: COc1cnc(NC(C)=O)cc1Nc1cc(-c2ccncc2F)nc(C(C)(F)F)n1] |
| | LCMS m/z = 419 [M + H]⁺. |
| 374 | N-(4-((6-(1-cyclobutyl-1H-pyrazol-4-yl)-2-(1,1-difluoroethyl)pyrimidin-4-yl)amino)-5- |
| | methoxypyridin-2-yl)acetamide |
| | 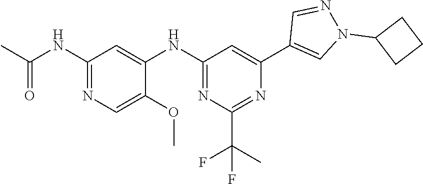 [SMILES: COc1cnc(NC(C)=O)cc1Nc1cc(-c2cnn(C3CCC3)c2)nc(C(C)(F)F)n1] |
| | LCMS m/z = 444 [M + H]⁺. |
| 375 | N-(4-((2-(1,1-difluoroethyl)-6-(1-(difluoromethyl)-1H-pyrazol-4-yl)pyrimidin-4-yl)amino)- |
| | 5-methoxypyridin-2-yl)acetamide |
| | 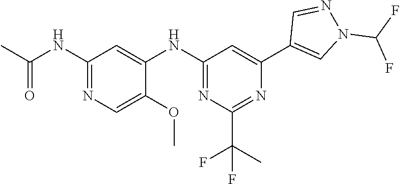 [SMILES: COc1cnc(NC(C)=O)cc1Nc1cc(-c2cnn(C(F)F)c2)nc(C(C)(F)F)n1] |
| | LCMS m/z = 440 [M + H]⁺. |
| 376 | N-(4-((6-(1-cyclopropyl-1H-pyrazol-4-yl)-2-(1,1-difluoroethyl)pyrimidin-4-yl)amino)-5- |
| | methoxypyridin-2-yl)acetamide |
| | 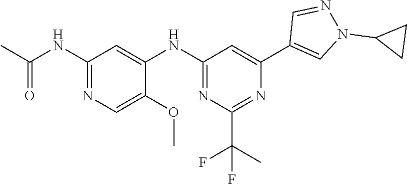 [SMILES: COc1cnc(NC(C)=O)cc1Nc1cc(-c2cnn(C3CC3)c2)nc(C(C)(F)F)n1] |
| | LCMS m/z = 430 [M + H]⁺ |
| 377 | N-(4-((2-(1,1-difluoroethyl)-6-(1-(oxetan-3-yl)-1H-pyrazol-4-yl)pyrimidin-4-yl)amino)-5- |
| | methoxypyridin-2-yl)acetamide |
| | 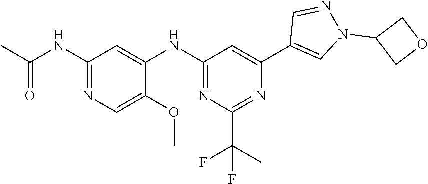 [SMILES: COc1cnc(NC(C)=O)cc1Nc1cc(-c2cnn(C3COC3)c2)nc(C(C)(F)F)n1] |
| | LCMS m/z = 446 [M + H]⁺. |
| 378 | N-(4-((6-(1-(cyclopropylmethyl)-1H-pyrazol-4-yl)-2-(1,1-difluoroethyl)pyrimidin-4- |
| | yl)amino)-5-methoxypyridin-2-yl)acetamide |
| | 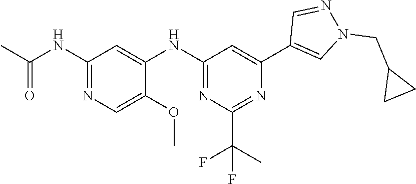 [SMILES: COc1cnc(NC(C)=O)cc1Nc1cc(-c2cnn(CC3CC3)c2)nc(C(C)(F)F)n1] |
| | LCMS m/z = 444 [M + H]⁺. |
| 379 | N-(4-((2-(1,1-difluoroethyl)-6-(1-(2-morpholinoethyl)-1H-pyrazol-4-yl)pyrimidin-4- |
| | yl)amino)-5-methoxypyridin-2-yl)acetamide |
| | 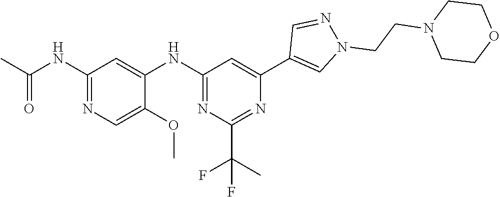 [SMILES: COc1cnc(NC(C)=O)cc1Nc1cc(-c2cnn(CCN3CCOCC3)c2)nc(C(C)(F)F)n1] |
| | LCMS m/z = 503 [M + H]⁺. |
| 380 | N-(4-((2-(1,1-difluoroethyl)-6-(1-(2-(dimethylamino)ethyl)-1H-pyrazol-4-yl)pyrimidin-4- |
| | yl)amino)-5-methoxypyridin-2-yl)acetamide |
| | 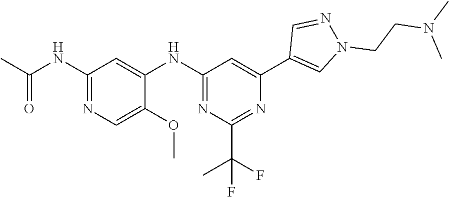 [SMILES: COc1cnc(NC(C)=O)cc1Nc1cc(-c2cnn(CCN(C)C)c2)nc(C(C)(F)F)n1] |
| | LCMS m/z = 461 [M + H]⁺. |
| 381 | N-(4-((2-(1,1-difluoroethyl)-6-(1-(2,2,2-trifluoroethyl)-1H-pyrazol-4-yl)pyrimidin-4- |
| | yl)amino)-5-methoxypyridin-2-yl)acetamide |
| | 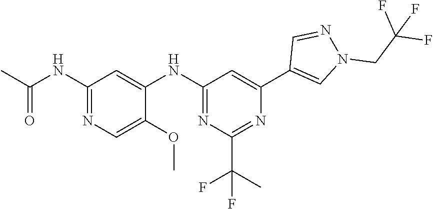 [SMILES: COc1cnc(NC(C)=O)cc1Nc1cc(-c2cnn(CC(F)(F)F)c2)nc(C(C)(F)F)n1] |
| | LCMS m/z = 472 [M + H]⁺. |
| 382 | N-(4-((2-(1,1-difluoroethyl)-6-(pyridin-4-yl)pyrimidin-4-yl)amino)-5-methoxypyridin-2- |
| | yl)acetamide |
| | 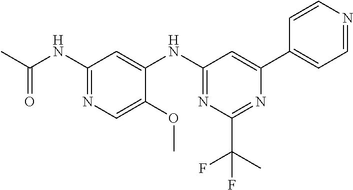 [SMILES: COc1cnc(NC(C)=O)cc1Nc1cc(-c2ccncc2)nc(C(C)(F)F)n1] |
| | LCMS m/z = 401 [M + H]⁺. |
| 383 | N-(4-((2-(1,1-difluoroethyl)-6-(5-fluoro-1-isopropyl-1H-pyrazol-4-yl)pyrimidin-4- |
| | yl)amino)-5-methoxypyridin-2-yl)acetamide |
| | 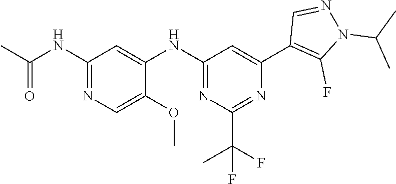 [SMILES: COc1cnc(NC(C)=O)cc1Nc1cc(-c2cnn(C(C)C)c2F)nc(C(C)(F)F)n1] |
| | LCMS m/z = 450 [M + H]⁺. |
| 384 | N-(4-((2-(1,1-difluoroethyl)-6-(5-fluoropyridin-2-yl)pyrimidin-4-yl)amino)-5- |
| | methoxypyridin-2-yl)acetamide |
| | 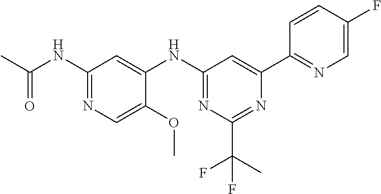 [SMILES: COc1cnc(NC(C)=O)cc1Nc1cc(-c2ccc(F)cn2)nc(C(C)(F)F)n1] |
| | LCMS m/z = 419 [M + H]⁺. |
| 385 | N-(4-((2-(1,1-difluoroethyl)-6-(5-fluoro-1H-pyrazol-4-yl)pyrimidin-4-yl)amino)-5- |
| | methoxypyridin-2-yl)acetamide |
| | 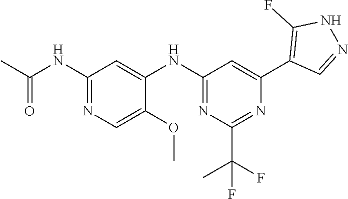 [SMILES: COc1cnc(NC(C)=O)cc1Nc1cc(-c2cn[nH]c2F)nc(C(C)(F)F)n1] |
| | LCMS m/z = 408 [M + H]⁺. |
| 386 | N-(4-((2-(1,1-difluoroethyl)-6-(1-(2-methoxyethyl)-1H-pyrazol-4-yl)pyrimidin-4-yl)amino)- |
| | 5-methoxypyridin-2-yl)acetamide |
| | 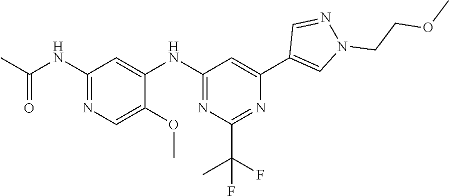 [SMILES: COCCn1cc(-c2cc(Nc3cc(NC(C)=O)ncc3OC)nc(C(C)(F)F)n2)cn1] |
| | LCMS m/z = 448 [M + H]⁺. |
| 387 | N-(4-((2-(1,1-difluoroethyl)-6-(2-methylthiazol-5-yl)pyrimidin-4-yl)amino)-5- |
| | methoxypyridin-2-yl)acetamide |
| | 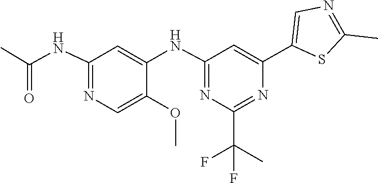 [SMILES: COc1cnc(NC(C)=O)cc1Nc1cc(-c2cnc(C)s2)nc(C(C)(F)F)n1] |
| | LCMS m/z = 421 [M + H]⁺. |
| 388 | N-(4-((2-(1,1-difluoroethyl)-6-(pyridin-3-yl)pyrimidin-4-yl)amino)-5-methoxypyridin-2- |
| | yl)acetamide |
| | 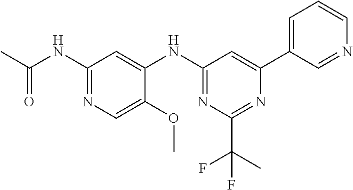 [SMILES: COc1cnc(NC(C)=O)cc1Nc1cc(-c2cccnc2)nc(C(C)(F)F)n1] |
| | LCMS m/z = 401 [M + H]⁺. |
| 389 | N-(4-((2-(1,1-difluoroethyl)-6-(5-methylpyridin-3-yl)pyrimidin-4-yl)amino)-5- |
| | methoxypyridin-2-yl)acetamide |
| | 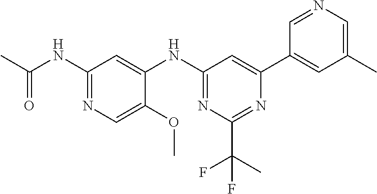 [SMILES: COc1cnc(NC(C)=O)cc1Nc1cc(-c2cncc(C)c2)nc(C(C)(F)F)n1] |
| | LCMS m/z = 415 [M + H]⁺. |
| 390 | N-(4-((2-(1,1-difluoroethyl)-2′-morpholino-[4,5′-bipyrimidin]-6-yl)amino)-5- |
| | methoxypyridin-2-yl)acetamide |
| | 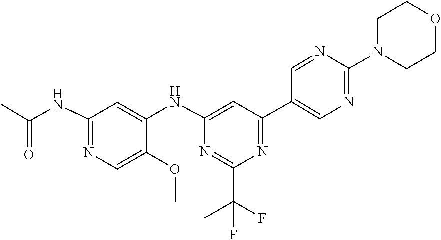 [SMILES: COc1cnc(NC(C)=O)cc1Nc1cc(-c2cnc(N3CCOCC3)nc2)nc(C(C)(F)F)n1] |
| | LCMS m/z = 487 [M + H]⁺. |
| 391 | N-(4-((2-(1,1-difluoroethyl)-6-(6-methoxypyridin-3-yl)pyrimidin-4-yl)amino)-5- |
| | methoxypyridin-2-yl)acetamide |
| | 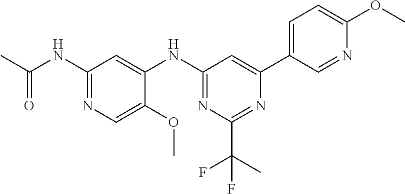 [SMILES: COc1ccc(-c2cc(Nc3cc(NC(C)=O)ncc3OC)nc(C(C)(F)F)n2)cn1] |
| | LCMS m/z = 431 [M + H]⁺. |
| 392 | N-(4-((2-(1,1-difluoroethyl)-6-(6-methoxypyridin-3-yl)pyrimidin-4-yl)amino)-5- |
| | methoxypyridin-2-yl)acetamide |
| | 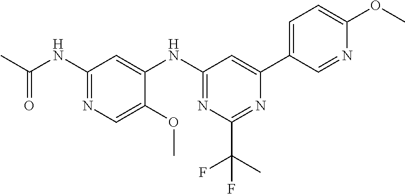 [SMILES: COc1ccc(-c2cc(Nc3cc(NC(C)=O)ncc3OC)nc(C(C)(F)F)n2)cn1] |
| | LCMS m/z = 446 [M + H]⁺. |
| 393 | N-(4-((6-(4-cyanophenyl)-2-(1,1-difluoroethyl)pyrimidin-4-yl)amino)-5-methoxypyridin-2- |
| | yl)acetamide |
| | 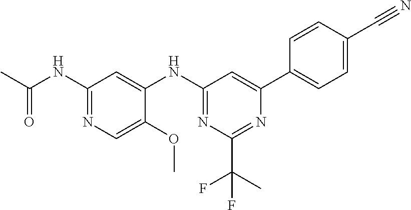 [SMILES: COc1cnc(NC(C)=O)cc1Nc1cc(-c2ccc(C#N)cc2)nc(C(C)(F)F)n1] |
| | LCMS m/z = 425 [M + H]⁺. |
| 394 | N-(4-((6-(3-cyano-4-fluorophenyl)-2-(1,1-difluoroethyl)pyrimidin-4-yl)amino)-5- |
| | methoxypyridin-2-yl)acetamide |
| | 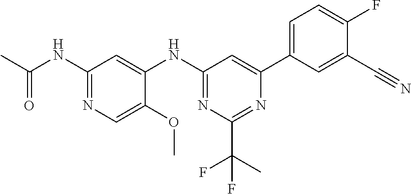 [SMILES: COc1cnc(NC(C)=O)cc1Nc1cc(-c2ccc(F)c(C#N)c2)nc(C(C)(F)F)n1] |
| | LCMS m/z = 443 [M + H]⁺. |
| 395 | N-(4-((2-(1,1-difluoroethyl)-6-(1-methyl-3-(trifluoromethyl)-1H-pyrazol-4-yl)pyrimidin-4- |
| | yl)amino)-5-methoxypyridin-2-yl)acetamide |
| | 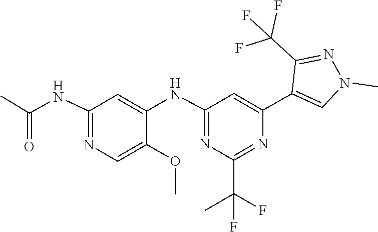 [SMILES: COc1cnc(NC(C)=O)cc1Nc1cc(-c2cn(C)nc2C(F)(F)F)nc(C(C)(F)F)n1] |
| | LCMS m/z = 472 [M + H]⁺. |
| 396 | N-(4-((2-(1,1-difluoroethyl)-6-(7-fluoro-2-methyl-2H-indazol-5-yl)pyrimidin-4-yl)amino)-5- |
| | methoxypyridin-2-yl)acetamide |
| | 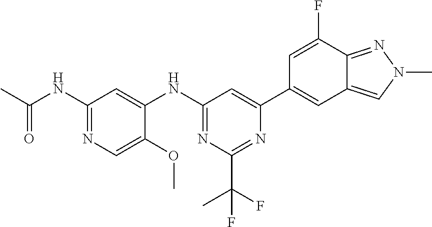 [SMILES: COc1cnc(NC(C)=O)cc1Nc1cc(-c2cc(F)c3nn(C)cc3c2)nc(C(C)(F)F)n1] |
| | LCMS m/z = 472 [M + H]⁺. |
| 397 | N-(4-((2-(1,1-difluoroethyl)-6-(2,8-dimethyl-[1,2,4]triazolo[1,5-a]pyridin-6-yl)pyrimidin-4- |
| | yl)amino)-5-methoxypyridin-2-yl)acetamide |
| | 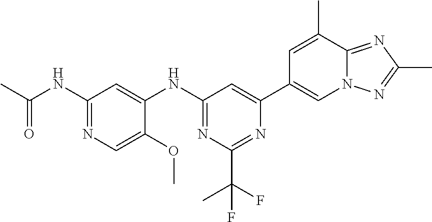 [SMILES: COc1cnc(NC(C)=O)cc1Nc1cc(-c2cc(C)c3nc(C)nn3c2)nc(C(C)(F)F)n1] |
| | LCMS m/z = 469 [M + H]⁺. |
| 398 | N-(4-((2-(1,1-difluoroethyl)-6-(8-fluoro-2-methylimidazo[1,2-a]pyridin-6-yl)pyrimidin-4- |
| | yl)amino)-5-methoxypyridin-2-yl)acetamide |
| | 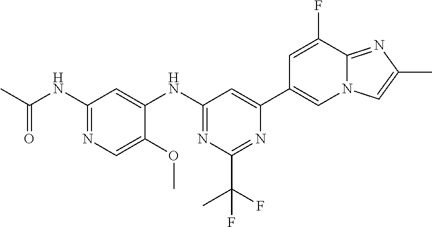 [SMILES: COc1cnc(NC(C)=O)cc1Nc1cc(-c2cc(F)c3nc(C)cn3c2)nc(C(C)(F)F)n1] |
| | LCMS m/z = 472 [M + H]⁺. |
| 399 | N-(4-((2-(1,1-difluoroethyl)-6-(2-methylimidazo[1,2-b]pyridazin-6-yl)pyrimidin-4- |
| | yl)amino)-5-methoxypyridin-2-yl)acetamide |
| | 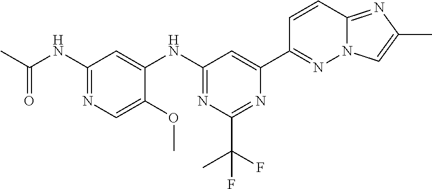 [SMILES: COc1cnc(NC(C)=O)cc1Nc1cc(-c2ccc3nc(C)cn3n2)nc(C(C)(F)F)n1] |
| | LCMS m/z = 455 [M + H]⁺. |
| 400 | N-(4-((2-(1,1-difluoroethyl)-6-(2,7-dimethyl-2H-indazol-5-yl)pyrimidin-4-yl)amino)-5- |
| | methoxypyridin-2-yl)acetamide |
| | 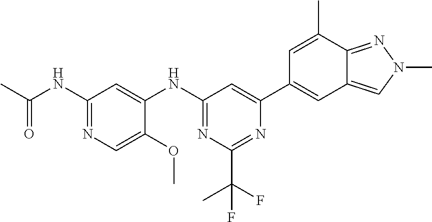 [SMILES: COc1cnc(NC(C)=O)cc1Nc1cc(-c2cc(C)c3nn(C)cc3c2)nc(C(C)(F)F)n1] |
| | LCMS m/z = 468 [M + H]⁺. |
| 401 | N-(4-((2-(1,1-difluoroethyl)-6-(8-methoxy-2-methylimidazo[1,2-a]pyridin-6-yl)pyrimidin-4- |
| | yl)amino)-5-methoxypyridin-2-yl)acetamide |
| | 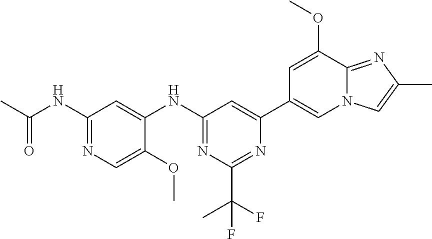 [SMILES: COc1cnc(NC(C)=O)cc1Nc1cc(-c2cc(OC)c3nc(C)cn3c2)nc(C(C)(F)F)n1] |
| | LCMS m/z = 484 [M + H]⁺. |
| 402 | N-(4-((6-(8-cyano-2-methylimidazo[1,2-a]pyridin-6-yl)-2-(1,1-difluoroethyl)pyrimidin-4- |
| | yl)amino)-5-methoxypyridin-2-yl)acetamide |
| | 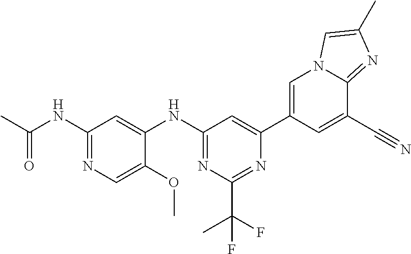 [SMILES: COc1cnc(NC(C)=O)cc1Nc1cc(-c2cc(C#N)c3nc(C)cn3c2)nc(C(C)(F)F)n1] |
| | LCMS m/z = 479 [M + H]⁺. |
| 403 | N-(4-((2-(1,1-difluoroethyl)-6-(2,8-dimethylimidazo[1,2-a]pyridin-6-yl)pyrimidin-4- |
| | yl)amino)-5-methoxypyridin-2-yl)acetamide |
| | 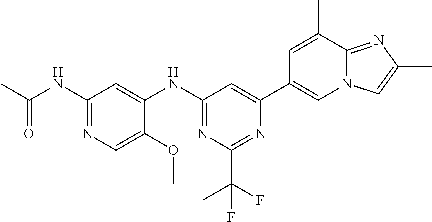 [SMILES: COc1cnc(NC(C)=O)cc1Nc1cc(-c2cc(C)c3nc(C)cn3c2)nc(C(C)(F)F)n1] |
| | LCMS m/z = 468 [M + H]⁺. |
| 404 | N-(4-((2-(1,1-difluoroethyl)-6-methylpyrimidin-4-yl)amino)-5-(2-fluoroethoxy)pyridin-2- |
| | yl)acetamide |
| | 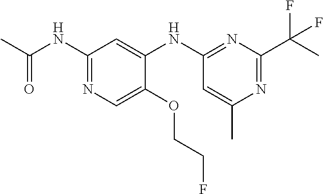 [SMILES: CC(=O)Nc1cc(Nc2cc(C)nc(C(C)(F)F)n2)c(OCCF)cn1] |
| | LCMS m/z = 370 [M + H]⁺. |
| 405 | N-(4-((2-(1,1-difluoroethyl)-6-ethylpyrimidin-4-yl)amino)-5-(3-fluoropropoxy)pyridin-2- |
| | yl)acetamide |
| | 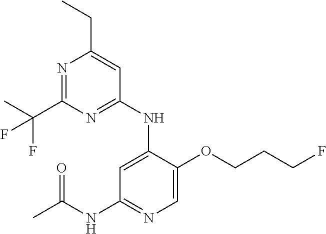 [SMILES: CCc1cc(Nc2cc(NC(C)=O)ncc2OCCCF)nc(C(C)(F)F)n1] |
| | LCMS m/z = 398.3 [M + H]⁺; ¹H NMR (600 MHz, DMSO-d₆) δ ppm 10.43 (br s, 1H), 9.17 |
| | (br s, 1H), 8.93 (br s, 1H), 8.02 (s, 1H), 7.19 (s, 1H), 4.60-4.73 (m, 2H), 4.23 (t, J = 6.3 |
| | Hz, 2H), 2.70 (m, 2H), 2.13-2.23 (m, 2H), 2.02-2.12 (m, 6H), 1.24 (t, J = 7.6 Hz, 3H) |
| 406 | N-(4-((2-(2-fluoropropan-2-yl)-6-methylpyrimidin-4-yl)amino)-5-(2- |
| | methoxyethoxy)pyridin-2-yl)acetamide |
| | 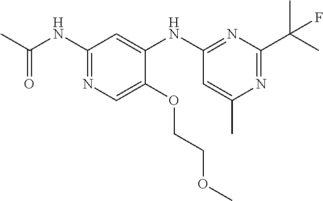 [SMILES: COCCOc1cnc(NC(C)=O)cc1Nc1cc(C)nc(C(C)(C)F)n1] |
| | LCMS m/z = 378.2 [M + H]⁺; ¹H NMR (400 MHz, CD₃OD): δ (ppm) 9.21 (s, 1H), 7.95 (s, |
| | 1H), 6.83 (s, 1H), 4.21-4.32 (m, 2H), 3.77-3.88 (m, 2H), 3.48 (s, 3H), 2.45 (s,3H), 2.16 (s, |
| | 3H), 1.72-1.86 (m, 6H) |
| 407 | N-(4-((2-(1,1-difluoroethyl)-6-ethylpyrimidin-4-yl)amino)-5-(2-fluoroethoxy)pyridin-2- |
| | yl)acetamide |
| | 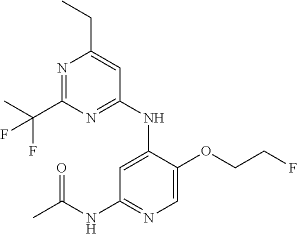 [SMILES: CCc1cc(Nc2cc(NC(C)=O)ncc2OCCF)nc(C(C)(F)F)n1] |
| | LCMS m/z = 384.1 [M + H]⁺; ¹H NMR (600 MHz, DMSO-d₆) δ ppm 10.37 (br s, 1H), 9.22 |
| | (br s, 1H), 8.98 (br s, 1H), 8.04 (s, 1H), 7.20 (s, 1H), 4.74-4.89 (m, 2H), 4.33-4.47 (m, |
| | 2H), 2.70 (m, 2H), 2.02-2.14 (m, 6H), 1.23 (t, J = 7.4 Hz, 3H) |
| 408 | N-(4-((2-(1,1-difluoroethyl)-6-isopropylpyrimidin-4-yl)amino)-5-((1r,3r)-3- |
| | methoxycyclobutoxy)pyridin-2-yl)acetamide |
| | 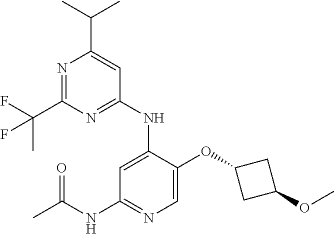 [SMILES: CO[C@H]1C[C@H](Oc2cnc(NC(C)=O)cc2Nc2cc(C(C)C)nc(C(C)(F)F)n2)C1] |
| | LCMS m/z = 436.2 [M + H]⁺. |
| 409 | N-(4-((2-(1,1-difluoroethyl)-6-isopropylpyrimidin-4-yl)amino)-5-((1s,3s)-3- |
| | methoxycyclobutoxy)pyridin-2-yl)acetamide |
| | 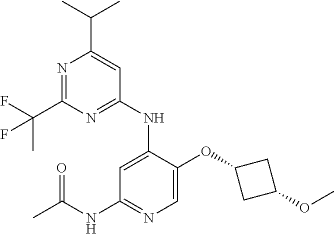 [SMILES: CO[C@H]1C[C@@H](Oc2cnc(NC(C)=O)cc2Nc2cc(C(C)C)nc(C(C)(F)F)n2)C1] |
| | LCMS m/z = 436.2 [M + H]⁺. |
| 410 | N-(4-((2-(1,1-difluoroethyl)-6-isopropylpyrimidin-4-yl)amino)-5-((1r,3r)-3- |
| | fluorocyclobutoxy)pyridin-2-yl)acetamide |
| | 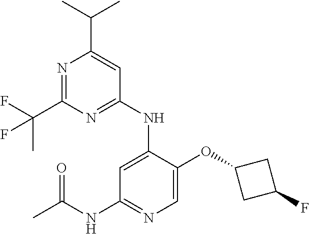 [SMILES: CC(=O)Nc1cc(Nc2cc(C(C)C)nc(C(C)(F)F)n2)c(O[C@H]2C[C@H](F)C2)cn1] |
| | LCMS m/z = 424.2 [M + H]⁺. |
| 411 | N-(4-((2-(1,1-difluoroethyl)-6-isopropylpyrimidin-4-yl)amino)-5-((1s,3s)-3- |
| | fluorocyclobutoxy)pyridin-2-yl)acetamide |
| | 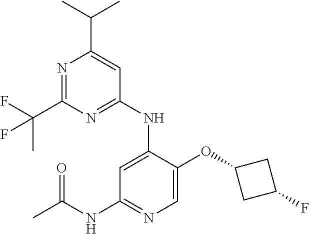 [SMILES: CC(=O)Nc1cc(Nc2cc(C(C)C)nc(C(C)(F)F)n2)c(O[C@H]2C[C@@H](F)C2)cn1] |
| | LCMS m/z = 424.1 [M + H]⁺. |
| 412 | N-(4-((2-(1,1-difluoroethyl)-6-methylpyrimidin-4-yl)amino)-5-((1s,3s)-3- |
| | fluorocyclobutoxy)pyridin-2-yl)acetamide |
| | 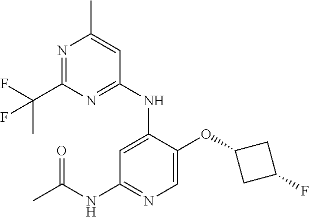 [SMILES: CC(=O)Nc1cc(Nc2cc(C)nc(C(C)(F)F)n2)c(O[C@H]2C[C@@H](F)C2)cn1] |
| | LCMS m/z = 396.1 [M + H]⁺. |
| 413 | N-(4-((2-(1,1-difluoroethyl)-6-ethylpyrimidin-4-yl)amino)-5-((1s,3s)-3- |
| | fluorocyclobutoxy)pyridin-2-yl)acetamide |
| | 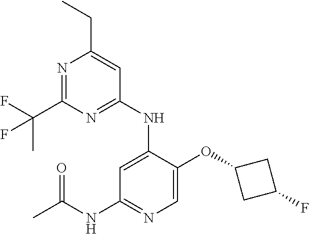 [SMILES: CCc1cc(Nc2cc(NC(C)=O)ncc2O[C@H]2C[C@@H](F)C2)nc(C(C)(F)F)n1] |
| | LCMS m/z = 410.2 [M + H]⁺. |
| 414 | N-(4-((2-(1,1-difluoroethyl)-6-(1-isopropyl-1H-pyrazol-4-yl)pyrimidin-4-yl)amino)-5- |
| | ((1s,3s)-3-fluorocyclobutoxy)pyridin-2-yl)acetamide |
| | 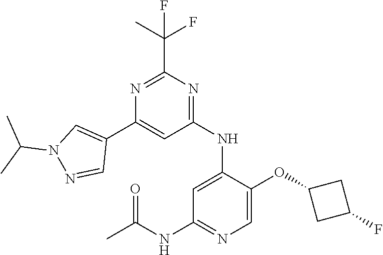 [SMILES: CC(=O)Nc1cc(Nc2cc(-c3cnn(C(C)C)c3)nc(C(C)(F)F)n2)c(O[C@H]2C[C@@H](F)C2)cn1] |
| | LCMS m/z = 490.2 [M + H]⁺. |
| 415 | N-(4-((6-cyclopropoxy-2-(1,1-difluoroethyl)pyrimidin-4-yl)amino)-5-(methoxy-d3)pyridin- |
| | 2-yl)acetamide |
| | 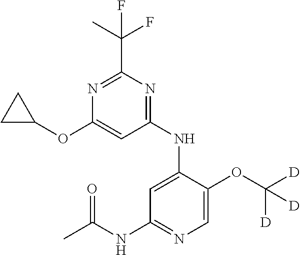 [SMILES: [2H]C([2H])([2H])Oc1cnc(NC(C)=O)cc1Nc1cc(OC2CC2)nc(C(C)(F)F)n1] |
| | LCMS m/z = 383.2[M + H]⁺; ¹H NMR (500 MHz, DMSO-d₆) δ ppm 10.34-10.17 (m, 1H), |
| | 9.44-9.31 (m, 1H), 9.01 (br s, 1H), 7.97 (br d, J = 2.4 Hz, 1H), 6.95-6.90 (m, 1H), 4.13 (br |
| | d, J = 3.1 Hz, 1H), 2.11-2.01 (m, 6H), 0.86-0.80 (m, 2H), 0.79-0.74 (m, 2H). |
| 416 | N-(4-((2-(1,1-difluoroethyl)-6-(1H-pyrazol-4-yl)pyrimidin-4-yl)amino)-5-methoxypyridin-2- |
| | yl)acetamide |
| | 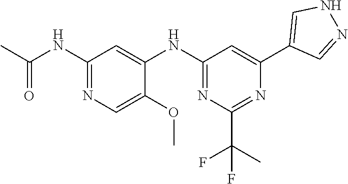 [SMILES: COc1cnc(NC(C)=O)cc1Nc1cc(-c2cn[nH]c2)nc(C(C)(F)F)n1] |
| | LCMS m/z = 390 [M + H]⁺. |
| 417 | N-(4-((2-(1,1-difluoroethyl)-6-methylpyrimidin-4-yl)amino)-5-((3-fluorotetrahydro-2H- |
| | pyran-3-yl)methoxy)pyridin-2-yl)acetamide |
| | 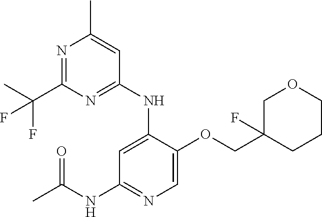 [SMILES: CC(=O)Nc1cc(Nc2cc(C)nc(C(C)(F)F)n2)c(OCC2(F)CCCOC2)cn1] |
| | LCMS m/z = 440 [M + H]⁺. |
| 418 | Cis-rac-N-(4-((2-(1,1-difluoroethyl)-6-methylpyrimidin-4-yl)amino)-5-(((2R,6R)-6- |
| | methyltetrahydro-2H-pyran-2-yl)methoxy)pyridin-2-yl)acetamide |
| | 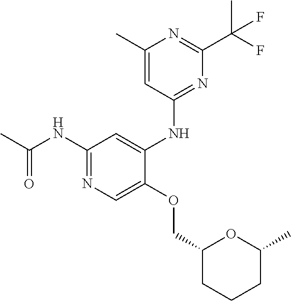 [SMILES: CC(=O)Nc1cc(Nc2cc(C)nc(C(C)(F)F)n2)c(OC[C@H]2CCC[C@@H](C)O2)cn1] |
| | LCMS m/z = 436 [M + H]⁺. |
| 419 | N-(4-((2-(1,1-difluoroethyl)-6-methylpyrimidin-4-yl)amino)-5-((6,6-dimethyl-1,4-dioxan-2- |
| | yl)methoxy)pyridin-2-yl)acetamide |
| | 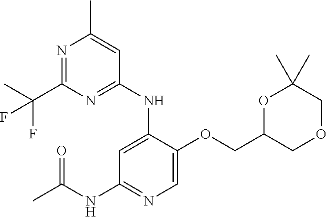 [SMILES: CC(=O)Nc1cc(Nc2cc(C)nc(C(C)(F)F)n2)c(OCC2COCC(C)(C)O2)cn1] |
| | LCMS m/z = 452 [M + H]⁺. |
| 420 | N-(5-(2-cyclobutyl-2-fluoroethoxy)-4-((2-(1,1-difluoroethyl)-6-methylpyrimidin-4- |
| | yl)amino)pyridin-2-yl)acetamide |
| | 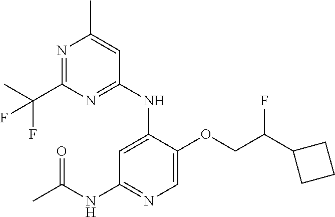 [SMILES: CC(=O)Nc1cc(Nc2cc(C)nc(C(C)(F)F)n2)c(OCC(F)C2CCC2)cn1] |
| | LCMS m/z = 424 [M + H]⁺. |
| 421 | N-(4-((2-(1,1-difluoroethyl)-6-methylpyrimidin-4-yl)amino)-5-(((1r,4s)-1-(fluoromethyl)-2- |
| | oxabicyclo[2.1.1]hexan-4-yl)methoxy)pyridin-2-yl)acetamide |
| | 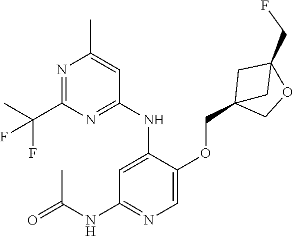 [SMILES: CC(=O)Nc1cc(Nc2cc(C)nc(C(C)(F)F)n2)c(OC[C@]23CO[C@](CF)(C2)C3)cn1] |
| | LCMS m/z = 452 [M + H]⁺. |
| 422 | N-(4-((2-(1,1-difluoroethyl)-6-methylpyrimidin-4-yl)amino)-5-(spiro[2.2]pentan-1- |
| | ylmethoxy)pyridin-2-yl)acetamide |
| | 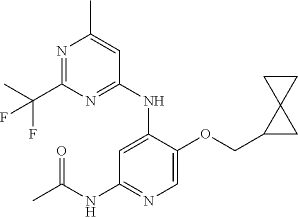 [SMILES: CC(=O)Nc1cc(Nc2cc(C)nc(C(C)(F)F)n2)c(OCC2CC23CC3)cn1] |
| | LCMS m/z = 404 [M + H]⁺. |
| 423 | N-(5-(((1r,4r)-7-oxabicyclo[2.2.1]heptan-1-yl)methoxy)-4-((2-(1,1-difluoroethyl)-6- |
| | methylpyrimidin-4-yl)amino)pyridin-2-yl)acetamide |
| | 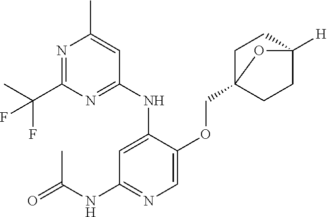 [SMILES: CC(=O)Nc1cc(Nc2cc(C)nc(C(C)(F)F)n2)c(OC[C@]23CC[C@@H](CC2)O3)cn1] |
| | LCMS m/z = 434 [M + H]⁺. |
| 424 | N-(4-((2-(1,1-difluoroethyl)-6-methylpyrimidin-4-yl)amino)-5-(2-methoxybutoxy)pyridin-2- |
| | yl)acetamide |
| | 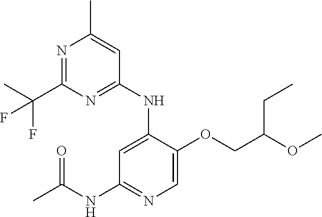 [SMILES: CCC(COc1cnc(NC(C)=O)cc1Nc1cc(C)nc(C(C)(F)F)n1)OC] |
| | LCMS m/z = 410 [M + H]⁺. |
| 425 | Cis-rac-N-(4-((2-(1,1-difluoroethyl)-6-methylpyrimidin-4-yl)amino)-5-((2- |
| | fluorocyclopropyl)methoxy)pyridin-2-yl)acetamide |
| | 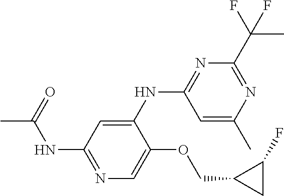 [SMILES: CC(=O)Nc1cc(Nc2cc(C)nc(C(C)(F)F)n2)c(OC[C@H]2C[C@H]2F)cn1] |
| | LCMS m/z = 396 [M + H]⁺. |
| 426 | Trans-rac-N-(4-((2-(1,1-difluoroethyl)-6-methylpyrimidin-4-yl)amino)-5-((2- |
| | fluorocyclopropyl)methoxy)pyridin-2-yl)acetamide |
| | 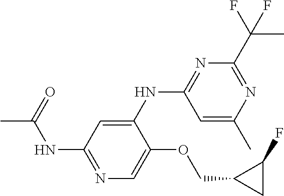 [SMILES: CC(=O)Nc1cc(Nc2cc(C)nc(C(C)(F)F)n2)c(OC[C@H]2C[C@@H]2F)cn1] |
| | LCMS m/z = 396 [M + H]⁺. |
| 427 | N-(5-(2-(tert-butoxy)propoxy)-4-((2-(1,1-difluoroethyl)-6-methylpyrimidin-4- |
| | yl)amino)pyridin-2-yl)acetamide |
| | 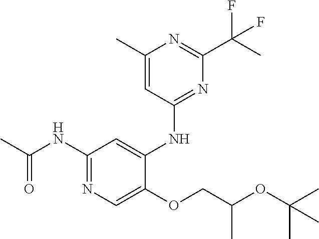 [SMILES: CC(=O)Nc1cc(Nc2cc(C)nc(C(C)(F)F)n2)c(OCC(C)OC(C)(C)C)cn1] |
| | LCMS m/z = 438 [M + H]⁺. |
| 428 | Cis-rac-N-(4-((2-(1,1-difluoroethyl)-6-methylpyrimidin-4-yl)amino)-5-((2- |
| | (fluoromethyl)cyclopropyl)methoxy)pyridin-2-yl)acetamide |
| | 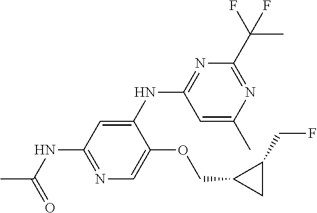 [SMILES: CC(=O)Nc1cc(Nc2cc(C)nc(C(C)(F)F)n2)c(OC[C@H]2C[C@H]2CF)cn1] |
| | LCMS m/z = 410 [M + H]⁺. |
| 429 | N-(4-((2-(1,1-difluoroethyl)-6-methylpyrimidin-4-yl)amino)-5-(2-fluoro-3- |
| | methylbutoxy)pyridin-2-yl)acetamide |
| | 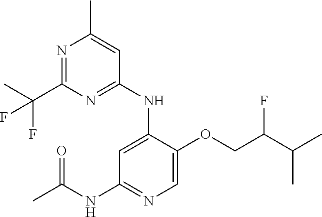 [SMILES: CC(=O)Nc1cc(Nc2cc(C)nc(C(C)(F)F)n2)c(OCC(F)C(C)C)cn1] |
| | LCMS m/z = 412 [M + H]⁺. |
| 430 | N-(4-((2-(1,1-difluoroethyl)-6-methylpyrimidin-4-yl)amino)-5-((5,5-dimethyl-1,4-dioxan-2- |
| | yl)methoxy)pyridin-2-yl)acetamide |
| | 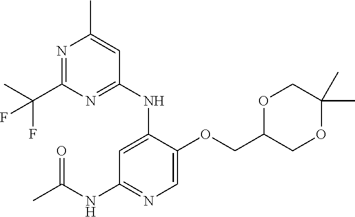 [SMILES: CC(=O)Nc1cc(Nc2cc(C)nc(C(C)(F)F)n2)c(OCC2COC(C)(C)CO2)cn1] |
| | LCMS m/z = 452 [M + H]⁺. |
| 431 | N-(4-((2-(1,1-difluoroethyl)-6-methylpyrimidin-4-yl)amino)-5-((3-methyltetrahydrofuran-3- |
| | yl)methoxy)pyridin-2-yl)acetamide |
| | 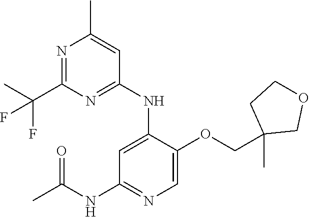 [SMILES: CC(=O)Nc1cc(Nc2cc(C)nc(C(C)(F)F)n2)c(OCC2(C)CCOC2)cn1] |
| | LCMS m/z = 422 [M + H]⁺. |
| 432 | N-(5-((5-oxaspiro[2.4]heptan-1-yl)methoxy)-4-((2-(1,1-difluoroethyl)-6-methylpyrimidin-4- |
| | yl)amino)pyridin-2-yl)acetamide |
| | 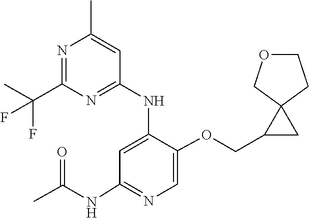 [SMILES: CC(=O)Nc1cc(Nc2cc(C)nc(C(C)(F)F)n2)c(OCC2CC23CCOC3)cn1] |
| | LCMS m/z = 434 [M + H]⁺. |
| 433 | N-(5-((6-oxaspiro[3.4]octan-7-yl)methoxy)-4-((2-(1,1-difluoroethyl)-6-methylpyrimidin-4- |
| | yl)amino)pyridin-2-yl)acetamide |
| | 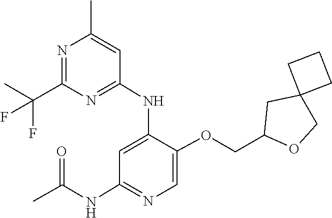 [SMILES: CC(=O)Nc1cc(Nc2cc(C)nc(C(C)(F)F)n2)c(OCC2CC3(CCC3)CO2)cn1] |
| | LCMS m/z = 448 [M + H]⁺. |
| 434 | N-(4-((2-(1,1-difluoroethyl)-6-methylpyrimidin-4-yl)amino)-5-(3-(tetrahydrofuran-2- |
| | yl)propoxy)pyridin-2-yl)acetamide |
| | 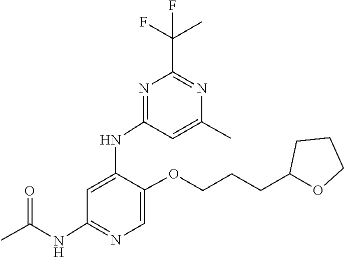 [SMILES: CC(=O)Nc1cc(Nc2cc(C)nc(C(C)(F)F)n2)c(OCCCC2CCCO2)cn1] |
| | LCMS m/z = 436 [M + H]⁺. |
| 435 | N-(4-((2-(1,1-difluoroethyl)-6-methylpyrimidin-4-yl)amino)-5-((2-methyltetrahydro-2H- |
| | pyran-2-yl)methoxy)pyridin-2-yl)acetamide |
| | 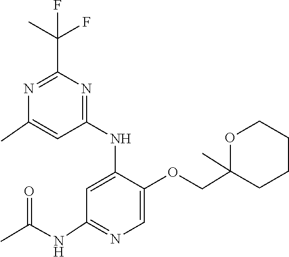 [SMILES: CC(=O)Nc1cc(Nc2cc(C)nc(C(C)(F)F)n2)c(OCC2(C)CCCCO2)cn1] |
| | LCMS m/z = 436 [M + H]⁺. |
| 436 | N-(5-methoxy-4-((4-methyl-6-(trifluoromethyl)pyrimidin-2-yl)amino)pyridin-2- |
| | yl)acetamide |
| | 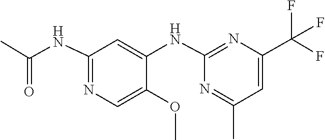 [SMILES: COc1cnc(NC(C)=O)cc1Nc1nc(C)cc(C(F)(F)F)n1] |
| | LCMS m/z = 342.2 [M + H]⁺; ¹H NMR (400 MHz, DMSO-d₆) δ ppm 10.21 (s, 1H), 9.10 (s, |
| | 1H), 8.71 (s, 1H), 7.99 (s, 1H), 7.38 (s, 1H), 3.91 (s, 3H), 2.54 (s, 3H), 2.05 (s, 3H). |
| 437 | N-(4-((6-cyclopropoxy-2-(trifluoromethyl)pyrimidin-4-yl)amino)-5-methoxypyridin-2- |
| | yl)acetamide |
| | 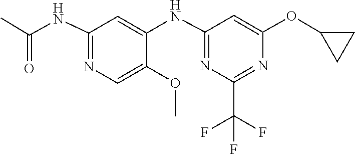 [SMILES: COc1cnc(NC(C)=O)cc1Nc1cc(OC2CC2)nc(C(F)(F)F)n1] |
| | LCMS m/z = 383.9 [M + H]⁺; ¹H NMR (500 MHz, DMSO-d₆) δ ppm 10.21 (s, 1H), 9.60 (s, |
| | 1H), 8.90 (br s, 1H), 8.03 (s, 1H), 6.94 (s, 1H), 4.15 (tt, J = 3.0, 6.0 Hz, 1H), 3.93 (s, 3H), |
| | 2.04 (s, 3H), 0.85-0.82 (m, 2H), 0.80-0.77 (m, 2H). |
| 438 | N-(5-methoxy-4-((4-methoxy-6-(trifluoromethyl)pyrimidin-2-yl)amino)pyridin-2- |
| | yl)acetamide |
| | 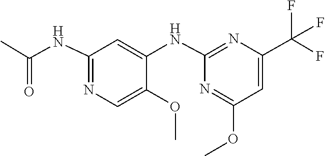 [SMILES: COc1cc(C(F)(F)F)nc(Nc2cc(NC(C)=O)ncc2OC)n1] |
| | LCMS m/z = 357.9 [M + H]⁺; ¹H NMR (500 MHz, DMSO-d₆) δ ppm 10.26 (s, 1H), 9.05 (s, |
| | 1H), 8.65 (s, 1H), 8.00 (s, 1H), 6.89 (s, 1H), 4.09 (s, 3H), 3.93 (s, 3H), 2.05 (s, 3H). |
| 439 | N-(4-((6-(2-fluoropropan-2-yl)pyrazin-2-yl)amino)-5-(2-methoxyethoxy)pyridin-2- |
| | yl)acetamide |
| | 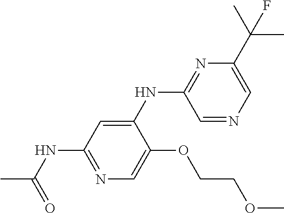 [SMILES: COCCOc1cnc(NC(C)=O)cc1Nc1cncc(C(C)(C)F)n1] |
| | LCMS m/z = 364.2 [M + H]⁺; ¹H NMR (400 MHz, CD₃OD) δ ppm 9.28 (s, 1H), 8.33 (s, 1H), |
| | 8.22 (d, J = 1.5 Hz, 1H), 7.92 (s, 1H), 4.24-4.33 (m, 2H), 3.79-3.90 (m, 2H), 3.50 (s, 3H), |
| | 2.15 (s, 3H), 1.82 (s, 3H), 1.76 (s, 3H). |
| 440 | N-(4-((2-(1,1-difluoroethyl)-6-methylpyrimidin-4-yl)amino)-5-(3-methoxy-2- |
| | methylpropoxy)pyridin-2-yl)acetamide |
| | 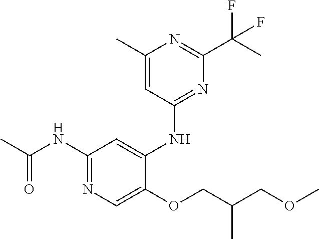 [SMILES: COCC(C)COc1cnc(NC(C)=O)cc1Nc1cc(C)nc(C(C)(F)F)n1] |
| | LCMS m/z = 410 [M + H]⁺. |
| 441 | N-(4-((4-(2-fluoropropan-2-yl)-6-methylpyrimidin-2-yl)amino)-5-(2- |
| | methoxyethoxy)pyridin-2-yl)acetamide |
| | 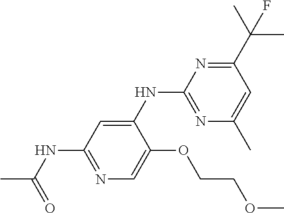 [SMILES: COCCOc1cnc(NC(C)=O)cc1Nc1nc(C)cc(C(C)(C)F)n1] |
| | LCMS m/z = 378.3 [M + H]⁺; 1H NMR (500 MHz, CD₃OD): δ ppm 9.36 (s, 1H), 7.91 (s, |
| | 1H), 7.05 (d, J = 1.5 Hz, 1H), 4.22-4.32 (m, 2H), 3.80 (dd, J = 5.0, 3.5 Hz, 2H), 3.48 (s, 3H), |
| | 2.49 (s, 3H), 2.16 (s, 3H), 1.68-1.82 (m, 6H) |
| 442 | Cis-rac-N-(4-((2-(1,1-difluoroethyl)-6-methylpyrimidin-4-yl)amino)-5-((6-methyltetrahydro- |
| | 2H-pyran-2-yl)methoxy)pyridin-2-yl)acetamide |
| | 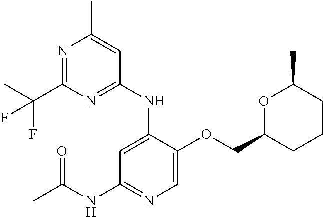 [SMILES: CC(=O)Nc1cc(Nc2cc(C)nc(C(C)(F)F)n2)c(OC[C@@H]2CCC[C@H](C)O2)cn1] |
| | LCMS m/z = 436 [M + H]⁺. |
| 443 | N-(5-((5,8-dioxaspiro[3.5]nonan-6-yl)methoxy)-4-((2-(1,1-difluoroethyl)-6- |
| | methylpyrimidin-4-yl)amino)pyridin-2-yl)acetamide |
| | 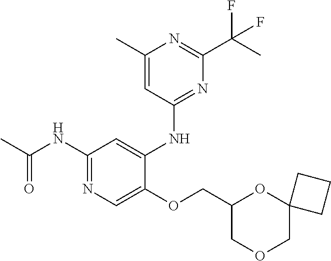 [SMILES: CC(=O)Nc1cc(Nc2cc(C)nc(C(C)(F)F)n2)c(OCC2COCC3(CCC3)O2)cn1] |
| | LCMS m/z = 464 [M + H]⁺. |
| 444 | N-(4-((2-(1,1-difluoroethyl)-6-methylpyrimidin-4-yl)amino)-5-(3-fluorobutoxy)pyridin-2- |
| | yl)acetamide |
| | 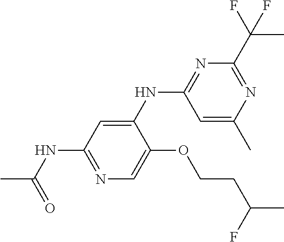 [SMILES: CC(=O)Nc1cc(Nc2cc(C)nc(C(C)(F)F)n2)c(OCCC(C)F)cn1] |
| | LCMS m/z = 398 [M + H]⁺. |
| 445 | N-(5-((1,4-dioxan-2-yl)methoxy)-4-((6-cyclopropoxy-2-(1,1-difluoroethyl)pyrimidin-4- |
| | yl)amino)pyridin-2-yl)acetamide |
| | 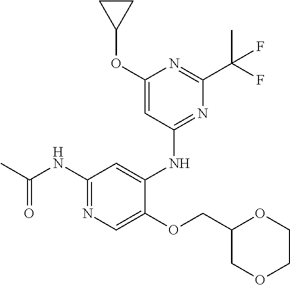 [SMILES: CC(=O)Nc1cc(Nc2cc(OC3CC3)nc(C(C)(F)F)n2)c(OCC2COCCO2)cn1] |
| | LCMS m/z = 466.2 [M + H]⁺. |
| 446 | N-(4-((6-cyclopropoxy-2-(2-fluoropropan-2-yl)pyrimidin-4-yl)amino)-5-(2- |
| | methoxyethoxy)pyridin-2-yl)acetamide |
| | 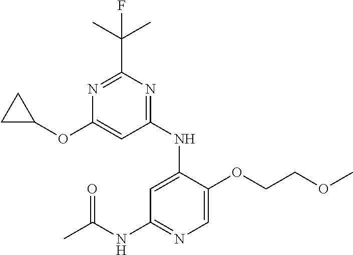 [SMILES: COCCOc1cnc(NC(C)=O)cc1Nc1cc(OC2CC2)nc(C(C)(C)F)n1] |
| | LCMS m/z = 420.2 [M + H]⁺. ¹H NMR (400 MHz, CD₃OD): δ ppm 9.10 (br s, 1H), 7.94 (s, |
| | 1H), 6.63 (s, 1H), 4.27 (br s, 2H), 4.13 (br dd, J = 3.8, 2.3 Hz, 1H), |
| | 3.77-3.88 (m, 2H), 3.48 (s, 3H), 2.15 (s, 3H), 1.79 (s, 3H), 1.73 (s, 3H), 0.77-0.93 (m, 4H) |
| 447 | methyl (4-((6-cyclopropoxy-2-(1,1-difluoroethyl)pyrimidin-4-yl)amino)-5-(2- |
| | (dimethylamino)ethoxy)pyridin-2-yl)carbamate |
| | 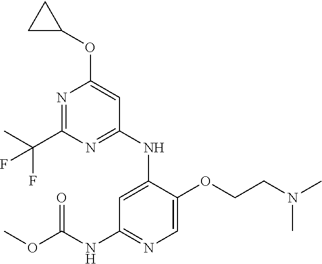 [SMILES: COC(=O)Nc1cc(Nc2cc(OC3CC3)nc(C(C)(F)F)n2)c(OCCN(C)C)cn1] |
| | LCMS m/z = 453.2 [M + H]⁺. |
| 448 | N-(4-((2-(2-oxabicyclo[2.1.1]hexan-4-yl)-6-methylpyrimidin-4-yl)amino)-5- |
| | methoxypyridin-2-yl)acetamide |
| | 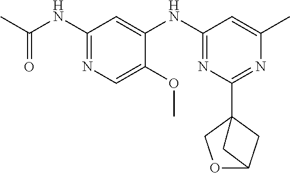 [SMILES: COc1cnc(NC(C)=O)cc1Nc1cc(C)nc(C23COC(C2)C3)n1] |
| | LCMS m/z = 356 [M + H]⁺. |
| 449 | N-(5-methoxy-4-((6-methyl-2-(1-methyl-2-oxabicyclo[2.1.1]hexan-4-yl)pyrimidin-4- |
| | yl)amino)pyridin-2-yl)acetamide |
| | 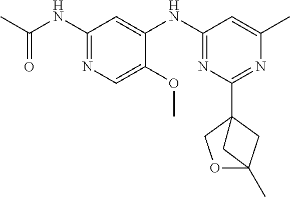 [SMILES: COc1cnc(NC(C)=O)cc1Nc1cc(C)nc(C23COC(C)(C2)C3)n1] |
| | LCMS m/z = 370 [M + H]⁺. |
| 450 | N-(5-methoxy-4-((2-(tetrahydrofuran-3-yl)pyrimidin-4-yl)amino)pyridin-2-yl)acetamide |
| | 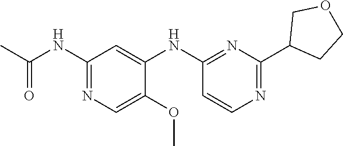 [SMILES: COc1cnc(NC(C)=O)cc1Nc1ccnc(C2CCOC2)n1] |
| | LCMS m/z= 330 [M + H]⁺; 1H NMR (600 MHz, DMSO-d₆): δ ppm 10.4-10.7 (m, 1H), 9.7- |
| | 10.0 (m, 1H), 9.00 (br s, 1H), 8.41 (d, 1H, J = 6.2 Hz), 8.02 (s, 1H), 7.2-7.2 (m, 1H), 4.11 (t, |
| | 1H, J = 8.1 Hz), 3.95 (s, 3H), 3.8-3.9 (m, 3H), 3.6-3.6 (m, 1H), 2.3-2.4 (m, 1H), 2.2-2.3 (m, |
| | 1H), 2.09 (s, 3H). |
| 451 | N-(5-methoxy-4-((6-methyl-2-(tetrahydrofuran-3-yl)pyrimidin-4-yl)amino)pyridin-2- |
| | yl)acetamide |
| | 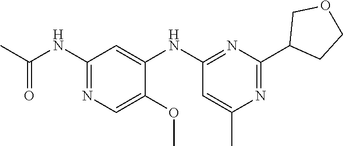 [SMILES: COc1cnc(NC(C)=O)cc1Nc1cc(C)nc(C2CCOC2)n1] |
| | LCMS m/z = 344 [M + H]⁺. |
| 452 | N-(5-methoxy-4-((2-methoxy-6-methylpyrimidin-4-yl)amino)pyridin-2-yl)acetamide |
| | 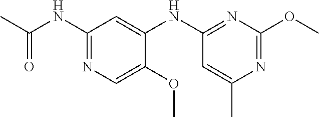 [SMILES: COc1nc(C)cc(Nc2cc(NC(C)=O)ncc2OC)n1] |
| | LCMS m/z = 304.1 [M + H]⁺. |
| 453 | (S)-N-(5-((1,4-dioxan-2-yl)methoxy)-4-((6-cyclopropoxy-2-(1,1-difluoroethyl)pyrimidin-4- |
| | yl)amino)pyridin-2-yl)acetamide or (R)-N-(5-((1,4-dioxan-2-yl)methoxy)-4-((6- |
| | cyclopropoxy-2-(1,1-difluoroethyl)pyrimidin-4-yl)amino)pyridin-2-yl)acetamide |
| | 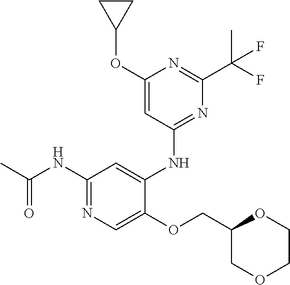 [SMILES: CC(=O)Nc1cc(Nc2cc(OC3CC3)nc(C(C)(F)F)n2)c(OC[C@@H]2COCCO2)cn1] |
| | Or |
| | 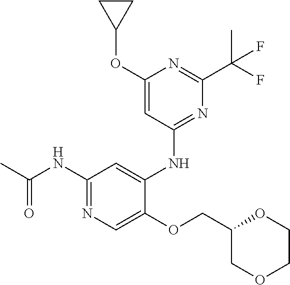 [SMILES: CC(=O)Nc1cc(Nc2cc(OC3CC3)nc(C(C)(F)F)n2)c(OC[C@H]2COCCO2)cn1] |
| | LCMS m/z = 466.2 [M + H]⁺. |
| 454 | (R)-N-(5-((1,4-dioxan-2-yl)methoxy)-4-((6-cyclopropoxy-2-(1,1-difluoroethyl)pyrimidin-4- |
| | yl)amino)pyridin-2-yl)acetamide or (S)-N-(5-((1,4-dioxan-2-yl)methoxy)-4-((6- |
| | cyclopropoxy-2-(1,1-difluoroethyl)pyrimidin-4-yl)amino)pyridin-2-yl)acetamide |
| | 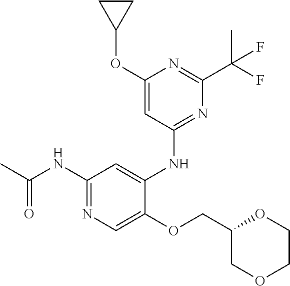 [SMILES: CC(=O)Nc1cc(Nc2cc(OC3CC3)nc(C(C)(F)F)n2)c(OC[C@H]2COCCO2)cn1] |
| | Or |
| | 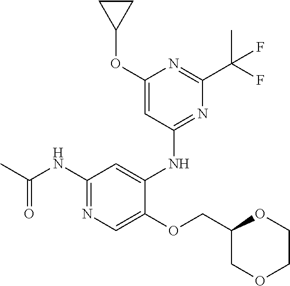 [SMILES: CC(=O)Nc1cc(Nc2cc(OC3CC3)nc(C(C)(F)F)n2)c(OC[C@@H]2COCCO2)cn1] |
| | LCMS m/z = 466.2 [M + H]⁺. |
| 455 | N-(4-((6-cyclobutoxy-2-(1,1-difluoroethyl)pyrimidin-4-yl)amino)-5-methoxypyridin-2- |
| | yl)acetamide trifluoroacetate |
| | 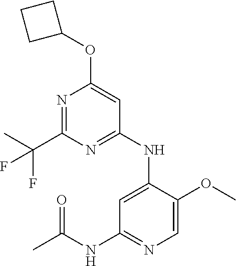 [SMILES: COc1cnc(NC(C)=O)cc1Nc1cc(OC2CCC2)nc(C(C)(F)F)n1] |
| | LCMS m/z = 394.2 [M + H]+ |
| 456 | N-(4-((6-(3-cyanocyclobutoxy)-2-(1,1-difluoroethyl)pyrimidin-4-yl)amino)-5- |
| | methoxypyridin-2-yl)acetamide |
| | 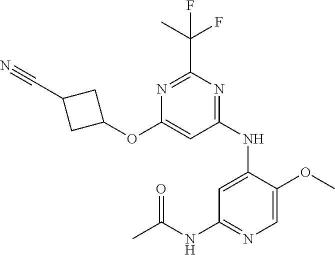 [SMILES: COc1cnc(NC(C)=O)cc1Nc1cc(OC2CC(C#N)C2)nc(C(C)(F)F)n1] |
| | LCMS m/z = 419.2 [M + H]+ |
| 457 | N-(4-((2-(1,1-difluoroethyl)-6-(3-fluorocyclobutoxy)pyrimidin-4-yl)amino)-5- |
| | ethoxypyridin-2-yl)acetamide trifluoroacetate |
| | 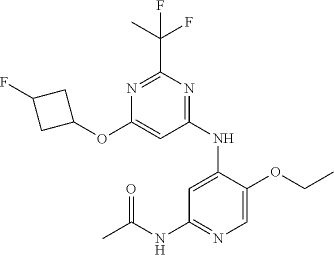 [SMILES: CCOc1cnc(NC(C)=O)cc1Nc1cc(OC2CC(F)C2)nc(C(C)(F)F)n1] |
| | LCMS m/z = 426.2 [M + H]+ |
| 458 | N-(4-((2-(1,1-difluoroethyl)-6-((1s,3s)-3-fluorocyclobutoxy)pyrimidin-4-yl)amino)-5- |
| | ethoxypyridin-2-yl)acetamide |
| | 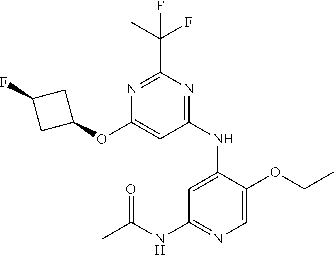 [SMILES: CCOc1cnc(NC(C)=O)cc1Nc1cc(O[C@H]2C[C@@H](F)C2)nc(C(C)(F)F)n1] |
| | LCMS m/z = 426.2 [M + H]+ |
| 459 | N-(4-((6-(3,3-difluorocyclobutoxy)-2-(1,1-difluoroethyl)pyrimidin-4-yl)amino)-5- |
| | ethoxypyridin-2-yl)acetamide |
| | 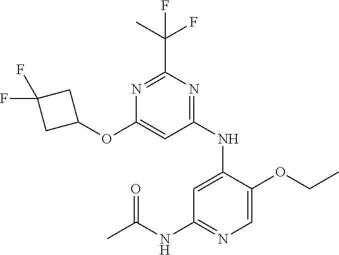 [SMILES: CCOc1cnc(NC(C)=O)cc1Nc1cc(OC2CC(F)(F)C2)nc(C(C)(F)F)n1] |
| | LCMS m/z = 444.2 [M + H]+ |
| 460 | N-(4-((6-(3,3-difluorocyclobutoxy)-2-(1,1-difluoroethyl)pyrimidin-4-yl)amino)-5- |
| | methoxypyridin-2-yl)acetamide |
| | 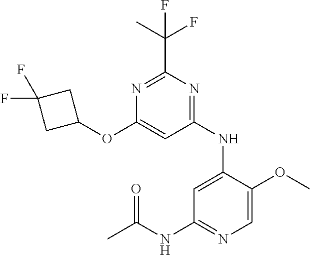 [SMILES: COc1cnc(NC(C)=O)cc1Nc1cc(OC2CC(F)(F)C2)nc(C(C)(F)F)n1] |
| | LCMS m/z = 430.1 [M + H]+ |
| 461 | N-(4-((2-(1,1-difluoroethyl)-6-((1s,3s)-3-fluorocyclobutoxy)pyrimidin-4-yl)amino)-5- |
| | methoxypyridin-2-yl)acetamide |
| | 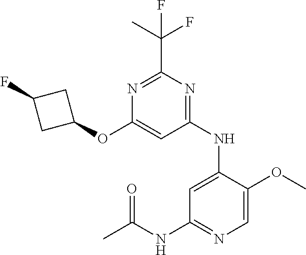 [SMILES: COc1cnc(NC(C)=O)cc1Nc1cc(O[C@H]2C[C@@H](F)C2)nc(C(C)(F)F)n1] |
| | LCMS m/z = 412.2 [M + H]+ |
| 462 | N-(4-((6-cyclobutoxy-2-(1,1-difluoroethyl)pyrimidin-4-yl)amino)-5-ethoxypyridin-2- |
| | yl)acetamide |
| | 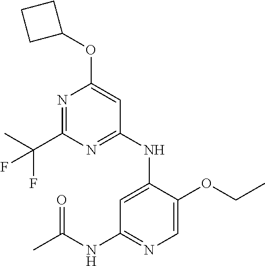 [SMILES: CCOc1cnc(NC(C)=O)cc1Nc1cc(OC2CCC2)nc(C(C)(F)F)n1] |
| | LCMS m/z = 408.2 [M + H]+ |
| 463 | N-(4-((6-(3-cyanocyclobutoxy)-2-(1,1-difluoroethyl)pyrimidin-4-yl)amino)-5- |
| | ethoxypyridin-2-yl)acetamide |
| | 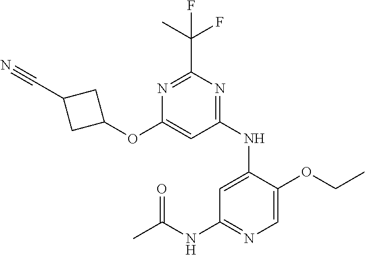 [SMILES: CCOc1cnc(NC(C)=O)cc1Nc1cc(OC2CC(C#N)C2)nc(C(C)(F)F)n1] |
| | LCMS m/z = 433.2 [M + H]+ |
| 464 | N-(4-((2-(1,1-difluoroethyl)-6-(3-methylcyclobutoxy)pyrimidin-4-yl)amino)-5- |
| | ethoxypyridin-2-yl)acetamide |
| | 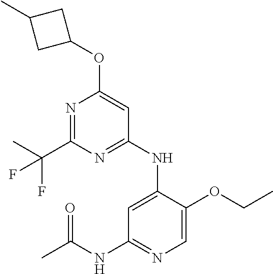 [SMILES: CCOc1cnc(NC(C)=O)cc1Nc1cc(OC2CC(C)C2)nc(C(C)(F)F)n1] |
| | LCMS m/z = 422.2 [M + H]+ |
| 465 | N-(5-((3,3-difluorocyclobutyl)methoxy)-4-((2-(1,1-difluoroethyl)-6-methylpyrimidin-4- |
| | yl)amino)pyridin-2-yl)acetamide |
| | 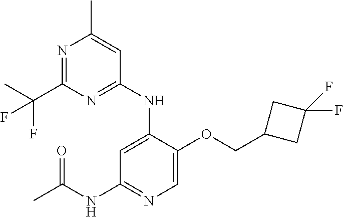 [SMILES: CC(=O)Nc1cc(Nc2cc(C)nc(C(C)(F)F)n2)c(OCC2CC(F)(F)C2)cn1] |
| | LCMS m/z = 428.2 [M + H]+ |
| 466 | N-(4-((2-(1,1-difluoroethyl)-6-methylpyrimidin-4-yl)amino)-5-((3- |
| | fluorobenzyl)oxy)pyridin-2-yl)acetamide |
| | 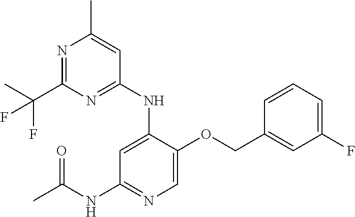 [SMILES: CC(=O)Nc1cc(Nc2cc(C)nc(C(C)(F)F)n2)c(OCc2cccc(F)c2)cn1] |
| | LCMS m/z = 432.2 [M + H]+ |
| 467 | N-(5-((3,4-difluorobenzyl)oxy)-4-((2-(1,1-difluoroethyl)-6-methylpyrimidin-4- |
| | yl)amino)pyridin-2-yl)acetamide |
| | 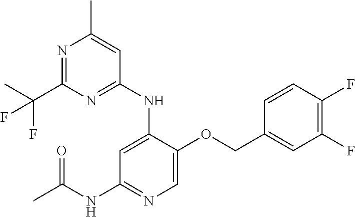 [SMILES: CC(=O)Nc1cc(Nc2cc(C)nc(C(C)(F)F)n2)c(OCc2ccc(F)c(F)c2)cn1] |
| | LCMS m/z = 450.2 [M + H]+ |
| 468 | N-(4-((2-(1,1-difluoroethyl)-6-methylpyrimidin-4-yl)amino)-5-((3- |
| | methoxybenzyl)oxy)pyridin-2-yl)acetamide |
| | 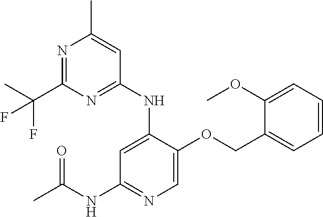 [SMILES: COc1ccccc1COc1cnc(NC(C)=O)cc1Nc1cc(C)nc(C(C)(F)F)n1] |
| | LCMS m/z = 444.2 [M + H]+ |
| 469 | N-(4-((2-(1,1-difluoroethyl)-6-methylpyrimidin-4-yl)amino)-5-((2-methylpyridin-3- |
| | yl)methoxy)pyridin-2-yl)acetamide |
| | 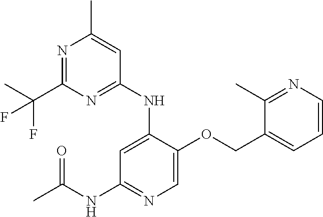 [SMILES: CC(=O)Nc1cc(Nc2cc(C)nc(C(C)(F)F)n2)c(OCc2cccnc2C)cn1] |
| | LCMS m/z = 429.2 [M + H]+ |
| 470 | N-(4-((2-(1,1-difluoroethyl)-6-methylpyrimidin-4-yl)amino)-5-((4-methylpyridin-3- |
| | yl)methoxy)pyridin-2-yl)acetamide |
| | 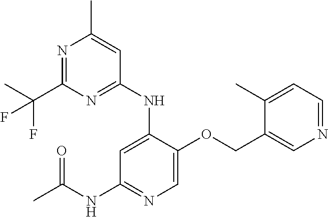 [SMILES: CC(=O)Nc1cc(Nc2cc(C)nc(C(C)(F)F)n2)c(OCc2cnccc2C)cn1] |
| | LCMS m/z = 429.2 [M + H]+ |
| 471 | N-(4-((2-(1,1-difluoroethyl)-6-methylpyrimidin-4-yl)amino)-5-((3-methylpyridin-2- |
| | yl)methoxy)pyridin-2-yl)acetamide |
| | 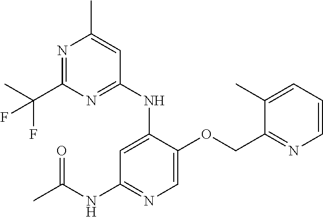 [SMILES: CC(=O)Nc1cc(Nc2cc(C)nc(C(C)(F)F)n2)c(OCc2ncccc2C)cn1] |
| | LCMS m/z = 429.2 [M + H]+ |
| 472 | N-(4-((2-(1,1-difluoroethyl)-6-methylpyrimidin-4-yl)amino)-5-((3-methoxypyridin-2- |
| | yl)methoxy)pyridin-2-yl)acetamide |
| | 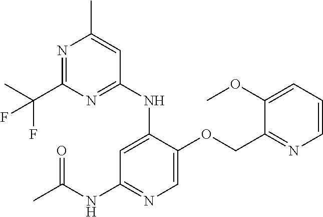 [SMILES: COc1cccnc1COc1cnc(NC(C)=O)cc1Nc1cc(C)nc(C(C)(F)F)n1] |
| | LCMS m/z = 445.2 [M + H]+ |
| 473 | N-(4-((2-(1,1-difluoroethyl)-6-methylpyrimidin-4-yl)amino)-5-((pyridin-4- |
| | yl)methoxy)pyridin-2-yl)acetamide |
| | 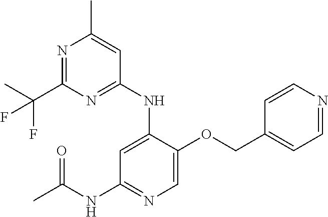 [SMILES: CC(=O)Nc1cc(Nc2cc(C)nc(C(C)(F)F)n2)c(OCc2ccncc2)cn1] |
| | LCMS m/z = 415.2 [M + H]+ |
| 474 | N-(4-((2-(1,1-difluoroethyl)-6-methylpyrimidin-4-yl)amino)-5-((5-fluoropyridin-3- |
| | yl)methoxy)pyridin-2-yl)acetamide |
| | 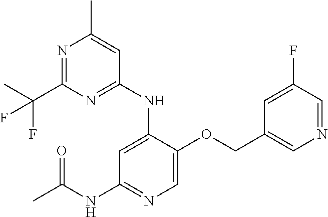 [SMILES: CC(=O)Nc1cc(Nc2cc(C)nc(C(C)(F)F)n2)c(OCc2cncc(F)c2)cn1] |
| | LCMS m/z = 433.2 [M + H]+ |
| 475 | N-(4-((2-(1,1-difluoroethyl)-6-methylpyrimidin-4-yl)amino)-5-((1-methyl-1H-pyrazol-3- |
| | yl)methoxy)pyridin-2-yl)acetamide |
| | 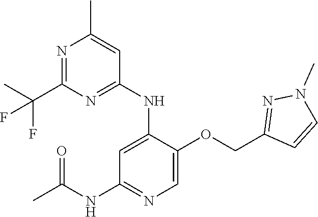 [SMILES: CC(=O)Nc1cc(Nc2cc(C)nc(C(C)(F)F)n2)c(OCc2ccn(C)n2)cn1] |
| | LCMS m/z = 418.2 [M + H]+ |
| 476 | N-(4-((2-(1,1-difluoroethyl)-6-methylpyrimidin-4-yl)amino)-5-(2-(pyridin-2- |
| | yl)ethoxy)pyridin-2-yl)acetamide |
| | 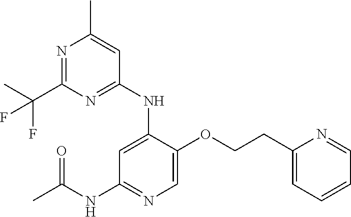 [SMILES: CC(=O)Nc1cc(Nc2cc(C)nc(C(C)(F)F)n2)c(OCCc2ccccn2)cn1] |
| | LCMS m/z = 429.2 [M + H]+ |
| 477 | N-(4-((2-(1,1-difluoroethyl)-6-methylpyrimidin-4-yl)amino)-5-((tetrahydrofuran-3- |
| | yl)oxy)pyridin-2-yl)acetamide |
| | 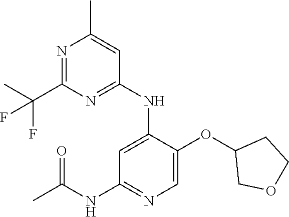 [SMILES: CC(=O)Nc1cc(Nc2cc(C)nc(C(C)(F)F)n2)c(OC2CCOC2)cn1] |
| | LCMS m/z = 394.2 [M + H]+ |
| 478 | N-(4-((2-(1,1-difluoroethyl)-6-methylpyrimidin-4-yl)amino)-5-((1-methylpyrrolidin-3- |
| | yl)oxy)pyridin-2-yl)acetamide |
| | 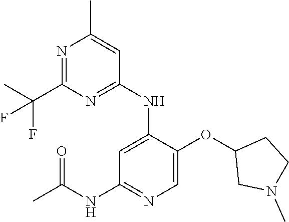 [SMILES: CC(=O)Nc1cc(Nc2cc(C)nc(C(C)(F)F)n2)c(OC2CCN(C)C2)cn1] |
| | LCMS m/z = 407.2 [M + H]+ |
| 479 | N-(4-((2-(1,1-difluoroethyl)-6-methylpyrimidin-4-yl)amino)-5-((1-methylpyrrolidin-2- |
| | yl)methoxy)pyridin-2-yl)acetamide |
| | 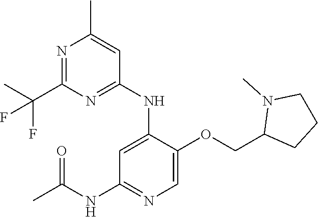 [SMILES: CC(=O)Nc1cc(Nc2cc(C)nc(C(C)(F)F)n2)c(OCC2CCCN2C)cn1] |
| | LCMS m/z = 421.2 [M + H]+ |
| 480 | N-(4-((2-(1,1-difluoroethyl)-6-methylpyrimidin-4-yl)amino)-5-((1-methylpyrrolidin-3- |
| | yl)methoxy)pyridin-2-yl)acetamide |
| | 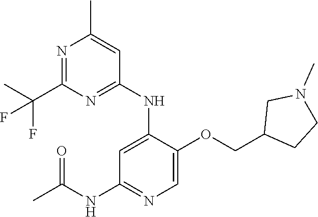 [SMILES: CC(=O)Nc1cc(Nc2cc(C)nc(C(C)(F)F)n2)c(OCC2CCN(C)C2)cn1] |
| | LCMS m/z = 421.2 [M + H]+ |
| 481 | N-(4-((2-(1,1-difluoroethyl)-6-methylpyrimidin-4-yl)amino)-5-((tetrahydrofuran-2- |
| | yl)methoxy)pyridin-2-yl)acetamide |
| | 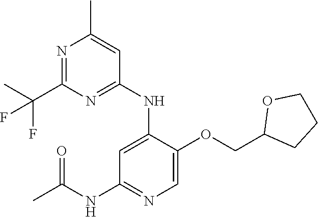 [SMILES: CC(=O)Nc1cc(Nc2cc(C)nc(C(C)(F)F)n2)c(OCC2CCCO2)cn1] |
| | LCMS m/z = 408.2 [M + H]+ |
| 482 | N-(4-((2-(1,1-difluoroethyl)-6-methylpyrimidin-4-yl)amino)-5-(2-isopropoxyethoxy)pyridin- |
| | 2-yl)acetamide |
| | 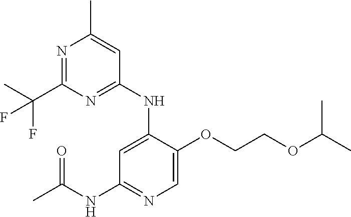 [SMILES: CC(=O)Nc1cc(Nc2cc(C)nc(C(C)(F)F)n2)c(OCCOC(C)C)cn1] |
| | LCMS m/z = 410.2 [M + H]+ |
| 483 | N-(4-((2-(1,1-difluoroethyl)-6-methylpyrimidin-4-yl)amino)-5-((3-fluorooxetan-3- |
| | yl)methoxy)pyridin-2-yl)acetamide |
| | 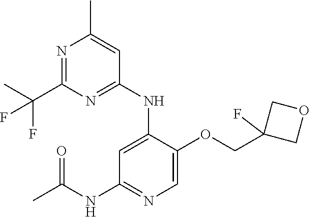 [SMILES: CC(=O)Nc1cc(Nc2cc(C)nc(C(C)(F)F)n2)c(OCC2(F)COC2)cn1] |
| | LCMS m/z = 412.2 [M + H]+ |
| 484 | N-(5-(benzo[d][1,3]dioxol-5-ylmethoxy)-4-((2-(1,1-difluoroethyl)-6-methylpyrimidin-4- |
| | yl)amino)pyridin-2-yl)acetamide |
| | 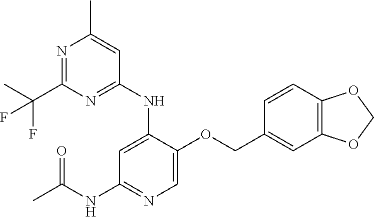 [SMILES: CC(=O)Nc1cc(Nc2cc(C)nc(C(C)(F)F)n2)c(OCc2ccc3c(c2)OCO3)cn1] |
| | LCMS m/z = 458.2 [M + H]+ |
| 485 | N-(4-((2-(1,1-difluoroethyl)-6-(methylamino)pyrimidin-4-yl)amino)-5-ethoxypyridin-2- |
| | yl)acetamide |
| | 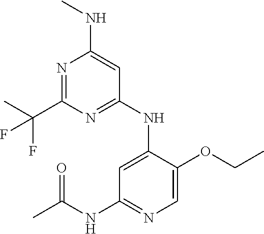 [SMILES: CCOc1cnc(NC(C)=O)cc1Nc1cc(NC)nc(C(C)(F)F)n1] |
| | LCMS m/z = 367.2 [M + H]+ |
| 486 | N-(4-((2-(1,1-difluoroethyl)-6-methylpyrimidin-4-yl)amino)-5-hydroxypyridin-2- |
| | yl)acetamide |
| | 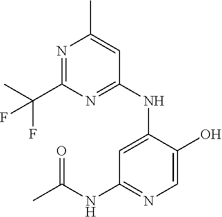 [SMILES: CC(=O)Nc1cc(Nc2cc(C)nc(C(C)(F)F)n2)c(O)cn1] |
| | LCMS m/z = 324.1 [M + H]+ |
| 487 | N-(5-(benzyloxy)-4-((6-cyclopropoxy-2-(1,1-difluoroethyl)pyrimidin-4-yl)amino)pyridin-2- |
| | yl)acetamide |
| | 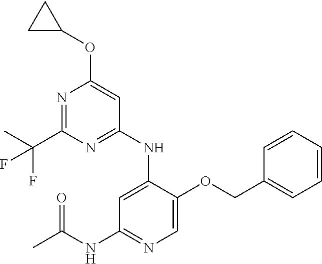 [SMILES: CC(=O)Nc1cc(Nc2cc(OC3CC3)nc(C(C)(F)F)n2)c(OCc2ccccc2)cn1] |
| | LCMS m/z = 456.2 [M + H]+ |
| 488 | N-(4-((6-(cyclobutylamino)-2-(1,1-difluoroethyl)pyrimidin-4-yl)amino)-5-(ethoxy- |
| | d5)pyridin-2-yl)acetamide |
| | 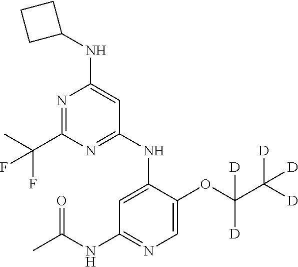 [SMILES: [2H]C([2H])([2H])C([2H])([2H])Oc1cnc(NC(C)=O)cc1Nc1cc(NC2CCC2)nc(C(C)(F)F)n1] |
| | LCMS m/z = 412.2 [M + H]+ |
| 489 | N-(4-((6-cyclopropoxy-2-(1,1-difluoroethyl)pyrimidin-4-yl)amino)-5-(ethoxy-d5)pyridin-2- |
| | yl)acetamide |
| | 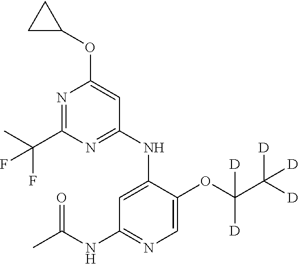 [SMILES: [2H]C([2H])([2H])C([2H])([2H])Oc1cnc(NC(C)=O)cc1Nc1cc(OC2CC2)nc(C(C)(F)F)n1] |
| | LCMS m/z = 399.2 [M + H]+ |
| 490 | N-(4-((6-cyclopropoxy-2-(1,1-difluoroethyl)pyrimidin-4-yl)amino)-5-ethoxypyridin-2- |
| | yl)acetamide |
| | 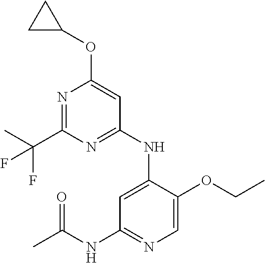 [SMILES: CCOc1cnc(NC(C)=O)cc1Nc1cc(OC2CC2)nc(C(C)(F)F)n1] |
| | LCMS m/z = 394.2 [M + H]+; ¹H NMR (DMSO-d₆) δ: 10.12-10.33 (m, 1H), 9.10-9.25 (m, |
| | 1H), 8.85-9.02 (m, 1H), 7.94-8.05 (m, 1H), 6.80-6.97 (m, 1H), 4.08-4.27 (m, 3H), 2.05 (s, |
| | 6H), 1.39 (s, 3H), 0.67-0.92 (m, 4H). |
| 491 | N-(5-ethoxy-4-((6-methoxy-5-(1-methyl-1H-pyrazol-3-yl)pyridin-2-yl)amino)pyridin-2- |
| | yl)acetamide |
| | 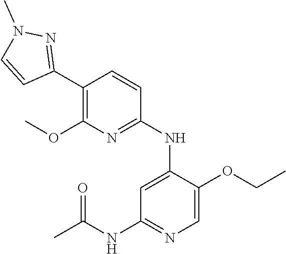 [SMILES: CCOc1cnc(NC(C)=O)cc1Nc1ccc(-c2ccn(C)n2)c(OC)n1] |
| | LCMS m/z = 383.2 [M + H]+; ¹H NMR (600 MHz, DMSO-d₆) δ (ppm) = 10.97 (br dd, J = |
| | 2.8, 6.0 Hz, 1H), 9.70-8.89 (m, 1H), 8.52 (br s, 1H), 8.23 (d, J = 8.0 Hz, 1H), 7.82 (s, 1H), |
| | 7.73 (d, J = 2.4 Hz, 1H), 7.11-6.99 (m, 1H), 6.71 (d, J = 2.4 Hz, 1H), 4.27-4.19 (m, 2H), |
| | 4.06 (s, 3H), 3.89 (s, 3H), 2.15 (s, 3H), 1.49-1.42 (m, 3H). |
| 492 | N-(4-((2-(1,1-difluoroethyl)-6-methylpyrimidin-4-yl)amino)-5-(2-methoxy-2- |
| | methylpropoxy)pyridin-2-yl)acetamide |
| | 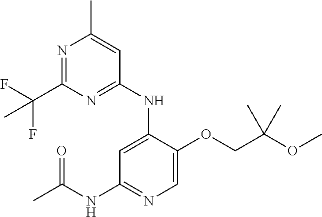 [SMILES: COC(C)(C)COc1cnc(NC(C)=O)cc1Nc1cc(C)nc(C(C)(F)F)n1] |
| | LCMS m/z = 410 [M + H]+ |
| 493 | N-(4-((2-(1,1-difluoroethyl)-6-methylpyrimidin-4-yl)amino)-5-((1- |
| | methoxycyclobutyl)methoxy)pyridin-2-yl)acetamide |
| | 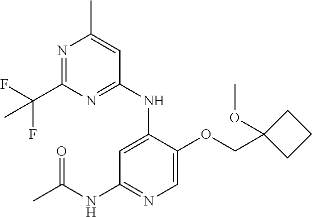 [SMILES: COC1(COc2cnc(NC(C)=O)cc2Nc2cc(C)nc(C(C)(F)F)n2)CCC1] |
| | LCMS m/z = 422 [M + H]+ |
| 494 | (R)-N-(5-((1-cyclopropylpyrrolidin-3-yl)methoxy)-4-((2-(1,1-difluoroethyl)-6- |
| | methylpyrimidin-4-yl)amino)pyridin-2-yl)acetamide |
| | 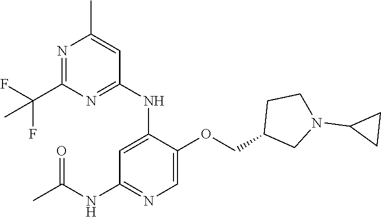 [SMILES: CC(=O)Nc1cc(Nc2cc(C)nc(C(C)(F)F)n2)c(OC[C@@H]2CCN(C3CC3)C2)cn1] |
| | LCMS m/z = 447 [M + H]+ |
| 495 | N-(4-((2-(1,1-difluoroethyl)-6-methylpyrimidin-4-yl)amino)-5-(isoxazol-3- |
| | ylmethoxy)pyridin-2-yl)acetamide |
| | 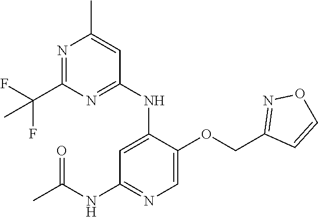 [SMILES: CC(=O)Nc1cc(Nc2cc(C)nc(C(C)(F)F)n2)c(OCc2ccon2)cn1] |
| | LCMS m/z = 405 [M + H]+ |
| 496 | N-(4-((2-(1,1-difluoroethyl)-6-methylpyrimidin-4-yl)amino)-5-((1-methyl-1H-imidazol-2- |
| | yl)methoxy)pyridin-2-yl)acetamide |
| | 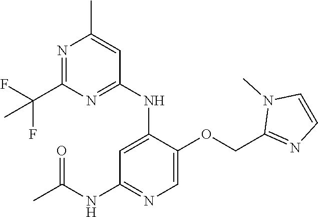 [SMILES: CC(=O)Nc1cc(Nc2cc(C)nc(C(C)(F)F)n2)c(OCc2nccn2C)cn1] |
| | LCMS m/z = 418 [M + H]+ |
| 497 | N-(4-((2-(1,1-difluoroethyl)-6-methylpyrimidin-4-yl)amino)-5-((5-methylisoxazol-3- |
| | yl)methoxy)pyridin-2-yl)acetamide |
| | 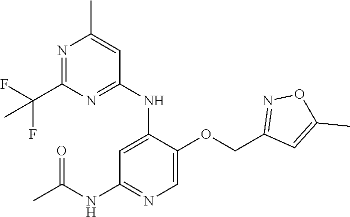 [SMILES: CC(=O)Nc1cc(Nc2cc(C)nc(C(C)(F)F)n2)c(OCc2cc(C)on2)cn1] |
| | LCMS m/z = 419 [M + H]+ |
| 498 | N-(4-((2-(1,1-difluoroethyl)-6-methylpyrimidin-4-yl)amino)-5-((5-methyl-1,2,4-oxadiazol- |
| | 3-yl)methoxy)pyridin-2-yl)acetamide |
| | 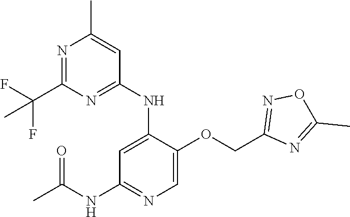 [SMILES: CC(=O)Nc1cc(Nc2cc(C)nc(C(C)(F)F)n2)c(OCc2noc(C)n2)cn1] |
| | LCMS m/z = 420 [M + H]+ |
| 499 | N-(4-((2-(1,1-difluoroethyl)-6-methylpyrimidin-4-yl)amino)-5-((5-oxotetrahydrofuran-2- |
| | yl)methoxy)pyridin-2-yl)acetamide |
| | 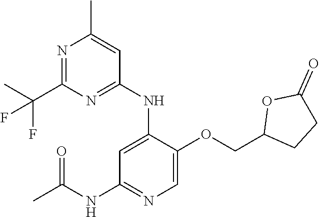 [SMILES: CC(=O)Nc1cc(Nc2cc(C)nc(C(C)(F)F)n2)c(OCC2CCC(=O)O2)cn1] |
| | LCMS m/z = 422 [M + H]+ |
| 500 | (S)-N-(4-((2-(1,1-difluoroethyl)-6-methylpyrimidin-4-yl)amino)-5-((5-oxopyrrolidin-2- |
| | yl)methoxy)pyridin-2-yl)acetamide |
| | 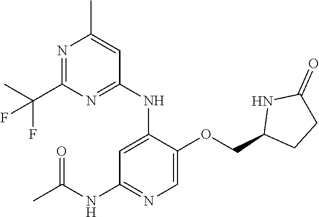 [SMILES: CC(=O)Nc1cc(Nc2cc(C)nc(C(C)(F)F)n2)c(OC[C@@H]2CCC(=O)N2)cn1] |
| | LCMS m/z = 421 [M + H]+ |
| 501 | N-(4-((2-(1,1-difluoroethyl)-6-methylpyrimidin-4-yl)amino)-5-(2-(1-methylpyrrolidin-2- |
| | yl)ethoxy)pyridin-2-yl)acetamide |
| | 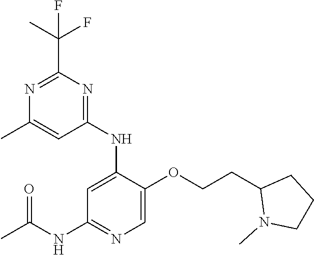 [SMILES: CC(=O)Nc1cc(Nc2cc(C)nc(C(C)(F)F)n2)c(OCCC2CCCN2C)cn1] |
| | LCMS m/z = 435 [M + H]+ |
| 502 | (R)-N-(4-((2-(1,1-difluoroethyl)-6-methylpyrimidin-4-yl)amino)-5-((5-oxopyrrolidin-2- |
| | yl)methoxy)pyridin-2-yl)acetamide |
| | 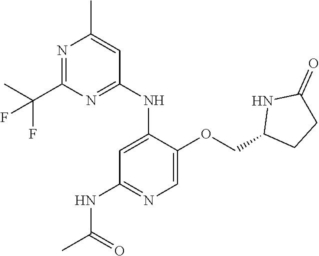 [SMILES: CC(=O)Nc1cc(Nc2cc(C)nc(C(C)(F)F)n2)c(OC[C@H]2CCC(=O)N2)cn1] |
| | LCMS m/z = 421 [M + H]+ |
| 503 | N-(4-((2-(1,1-difluoroethyl)-6-methylpyrimidin-4-yl)amino)-5-((4-isopropylmorpholin-2- |
| | yl)methoxy)pyridin-2-yl)acetamide |
| | 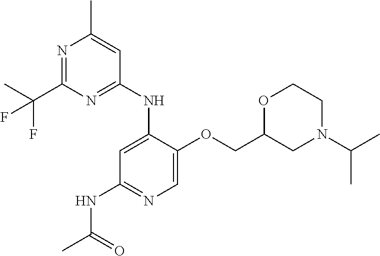 [SMILES: CC(=O)Nc1cc(Nc2cc(C)nc(C(C)(F)F)n2)c(OCC2CN(C(C)C)CCO2)cn1] |
| | LCMS m/z = 465 [M + H]+ |
| 504 | N-(4-((2-(1,1-difluoroethyl)-6-methylpyrimidin-4-yl)amino)-5-(isoxazol-4- |
| | ylmethoxy)pyridin-2-yl)acetamide |
| | 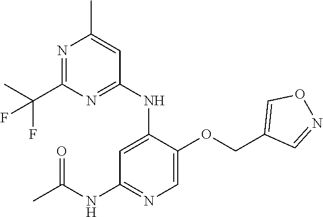 [SMILES: CC(=O)Nc1cc(Nc2cc(C)nc(C(C)(F)F)n2)c(OCc2cnoc2)cn1] |
| | LCMS m/z = 405 [M + H]+ |
| 505 | N-(4-((2-(1,1-difluoroethyl)-6-methylpyrimidin-4-yl)amino)-5-(oxazol-4- |
| | ylmethoxy)pyridin-2-yl)acetamide |
| | 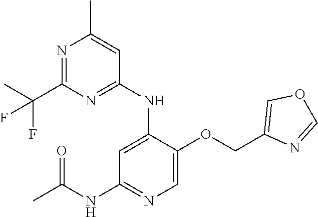 [SMILES: CC(=O)Nc1cc(Nc2cc(C)nc(C(C)(F)F)n2)c(OCc2cocn2)cn1] |
| | LCMS m/z = 405 [M + H]+ |
| 506 | N-(4-((2-(1,1-difluoroethyl)-6-methylpyrimidin-4-yl)amino)-5-((1-methylazetidin-3- |
| | yl)methoxy)pyridin-2-yl)acetamide |
| | 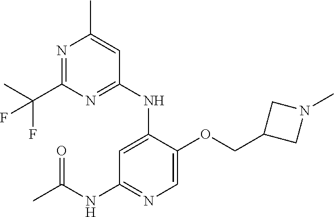 [SMILES: CC(=O)Nc1cc(Nc2cc(C)nc(C(C)(F)F)n2)c(OCC2CN(C)C2)cn1] |
| | LCMS m/z = 407 [M + H]+ |
| 507 | N-(4-((2-(1,1-difluoroethyl)-6-methylpyrimidin-4-yl)amino)-5-(2-ethoxyethoxy)pyridin-2- |
| | yl)acetamide |
| | 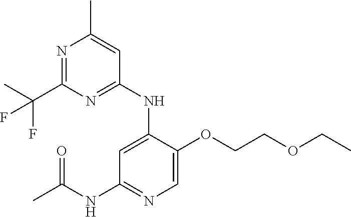 [SMILES: CCOCCOc1cnc(NC(C)=O)cc1Nc1cc(C)nc(C(C)(F)F)n1] |
| | LCMS m/z = 396 [M + H]+ |
| 508 | N-(4-((2-(1,1-difluoroethyl)-6-methylpyrimidin-4-yl)amino)-5-((1-methyl-1H-pyrazol-4- |
| | yl)methoxy)pyridin-2-yl)acetamide |
| | 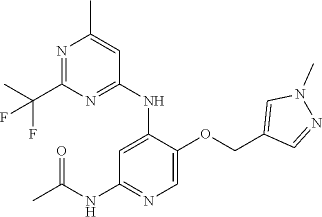 [SMILES: CC(=O)Nc1cc(Nc2cc(C)nc(C(C)(F)F)n2)c(OCc2cnn(C)c2)cn1] |
| | LCMS m/z = 418 [M + H]+ |
| 509 | N-(4-((2-(1,1-difluoroethyl)-6-methylpyrimidin-4-yl)amino)-5-((1-ethyl-1H-pyrazol-4- |
| | yl)methoxy)pyridin-2-yl)acetamide |
| | 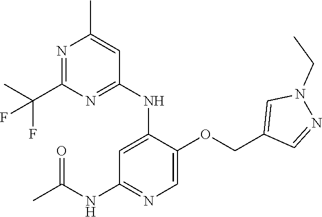 [SMILES: CCn1cc(COc2cnc(NC(C)=O)cc2Nc2cc(C)nc(C(C)(F)F)n2)cn1] |
| | LCMS m/z = 432 [M + H]+ |
| 510 | N-(4-((2-(1,1-difluoroethyl)-6-methylpyrimidin-4-yl)amino)-5-((1-isopropyl-5- |
| | oxopyrrolidin-3-yl)methoxy)pyridin-2-yl)acetamide |
| | 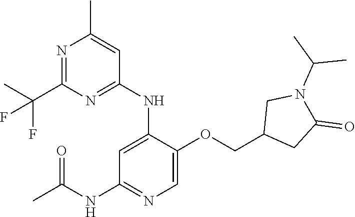 [SMILES: CC(=O)Nc1cc(Nc2cc(C)nc(C(C)(F)F)n2)c(OCC2CC(=O)N(C(C)C)C2)cn1] |
| | LCMS m/z = 463 [M + H]+ |
| 511 | N-(4-((2-(1,1-difluoroethyl)-6-methylpyrimidin-4-yl)amino)-5-((1-methyl-5-oxopyrrolidin- |
| | 3-yl)methoxy)pyridin-2-yl)acetamide |
| | 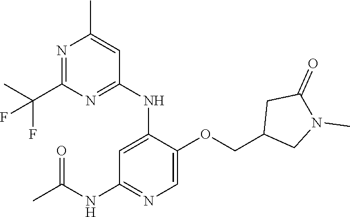 [SMILES: CC(=O)Nc1cc(Nc2cc(C)nc(C(C)(F)F)n2)c(OCC2CC(=O)N(C)C2)cn1] |
| | LCMS m/z = 435 [M + H]+ |
| 512 | N-(4-((2-(1,1-difluoroethyl)-6-methylpyrimidin-4-yl)amino)-5-((3-methoxy-1-methyl-1H- |
| | pyrazol-4-yl)methoxy)pyridin-2-yl)acetamide |
| | 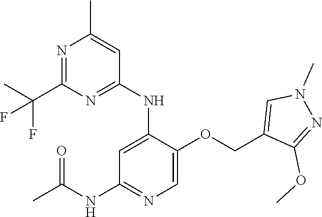 [SMILES: COc1nn(C)cc1COc1cnc(NC(C)=O)cc1Nc1cc(C)nc(C(C)(F)F)n1] |
| | LCMS m/z = 448 [M + H]+ |
| 513 | N-(4-((2-(1,1-difluoroethyl)-6-methylpyrimidin-4-yl)amino)-5-(((1R,3S)-3- |
| | methoxycyclopentyl)oxy)pyridin-2-yl)acetamide |
| | 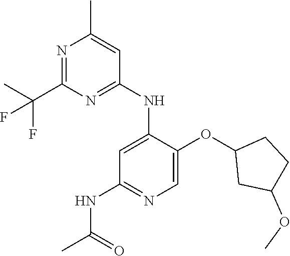 [SMILES: COC1CCC(Oc2cnc(NC(C)=O)cc2Nc2cc(C)nc(C(C)(F)F)n2)C1] |
| | LCMS m/z = 422 [M + H]+ |
| 514 | N-(4-((2-(1,1-difluoroethyl)-6-methylpyrimidin-4-yl)amino)-5-(((1s,4s)-4-methyl-2- |
| | oxabicyclo[2.1.1]hexan-1-yl)methoxy)pyridin-2-yl)acetamide |
| | 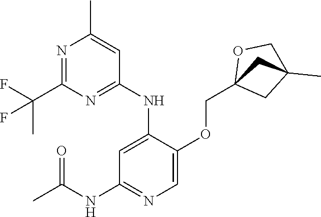 [SMILES: CC(=O)Nc1cc(Nc2cc(C)nc(C(C)(F)F)n2)c(OC[C@]23C[C@](C)(CO2)C3)cn1] |
| | LCMS m/z = 434 [M + H]+ |
| 515 | N-(4-((2-(1,1-difluoroethyl)-6-ethylpyrimidin-4-yl)amino)-5-(ethoxy-d5)pyridin-2- |
| | yl)acetamide |
| | 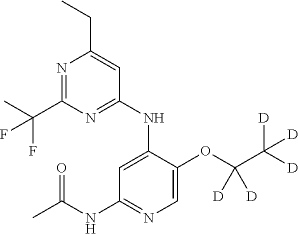 [SMILES: [2H]C([2H])([2H])C([2H])([2H])Oc1cnc(NC(C)=O)cc1Nc1cc(CC)nc(C(C)(F)F)n1] |
| | LCMS m/z = NO DATA |
| 516 | N-(4-((2-(1,1-difluoropropyl)-6-methylpyrimidin-4-yl)amino)-5-ethoxypyridin-2- |
| | yl)acetamide |
| | 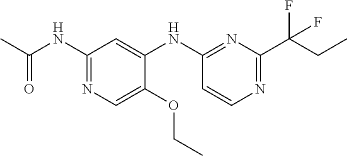 [SMILES: CCOc1cnc(NC(C)=O)cc1Nc1ccnc(C(F)(F)CC)n1] |
| | LCMS m/z = 366.2 [M + H]+ |
| 517 | N-(4-((2-(1,1-difluoroethyl)-5-methoxypyrimidin-4-yl)amino)-5-ethoxypyridin-2- |
| | yl)acetamide |
| | 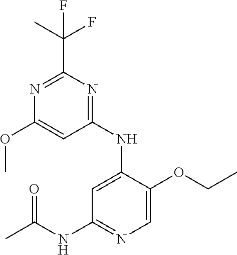 [SMILES: CCOc1cnc(NC(C)=O)cc1Nc1cc(OC)nc(C(C)(F)F)n1] |
| | LCMS m/z = 368.2 [M + H]+ |
| 518 | rac-(R)-N-(4-((2-(2,2-dimethylcyclopropyl)-6-methylpyrimidin-4-yl)amino)-5- |
| | ethoxypyridin-2-yl)acetamide |
| | 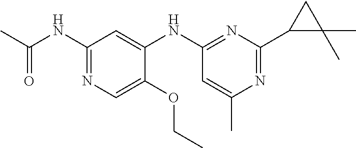 [SMILES: CCOc1cnc(NC(C)=O)cc1Nc1cc(C)nc(C2CC2(C)C)n1] |
| | LCMS m/z = 356.1 [M + H]+ |
| 519 | N-(5-ethoxy-4-((6-((1r,3r)-3-methoxycyclobutoxy)-2-methylpyrimidin-4-yl)amino)pyridin- |
| | 2-yl)acetamide |
| | 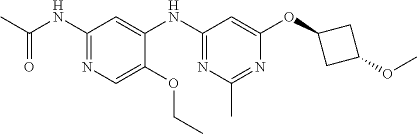 [SMILES: CCOc1cnc(NC(C)=O)cc1Nc1cc(O[C@H]2C[C@H](OC)C2)nc(C)n1] |
| | LCMS m/z = 388.1 [M + H]+ |

### Examples 520 to 922

The compounds in the table below were made using a similar process to the Examples described above.

| Ex. | |
|---|---|
| No. | Name/Structure/Data |
| 520 | 1-(5-cyano-4′-((2-(1,1-difluoroethyl)-6-methylpyrimidin-4-yl)amino)-[2,3′-bipyridin]-6′-yl)- 3-methylurea 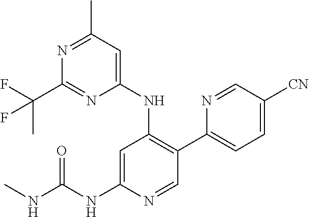 [SMILES: CNC(=O)Nc1cc(Nc2cc(C)nc(C(C)(F)F)n2)c(-c2ccc(C#N)cn2)cn1] SM: 6′-chloro-4′-((2-(1,1-difluoroethyl)-6-methylpyrimidin-4-yl)amino)-[2,3′-bipyridine]-5- carbonitrile (Preparation X) and 1-methylurea (Column: Phenomenex C18 150*25 mm*10 um; Condition: water (NH₄HCO₃)-ACN; B%: 22%-52%, 10 min) 14.2 mg, 17.3% yield as a white solid. LCMS m/z = 425.2 [M + H]⁺ 1H NMR (400 MHz, DMSO-d₆) δ ppm 11.76 (s, 1H), 9.53 (s, 1H), 9.14 (d, J = 1.6 Hz, 1H), 8.70 (s, 1H), 8.40 (dd, J = 8.6 Hz, 2.4 Hz, 1H), 8.36 (s, 1H), 8.14 (d, J = 8.4 Hz, 1H), 7.75- 7.82 (m, 1H), 7.07 (s, 1H), 2.75 (d, J = 4.8 Hz, 3H), 2.43 (s, 3H), 2.08 (t, J = 18.8 Hz, 3H). |
| 521 | N-(4-((2-(1,1-difluoroethyl)-6-ethylpyrimidin-4-yl)amino)-5-(4-methyl-1,3,5-triazin-2- yl)pyridin-2-yl)acetamide 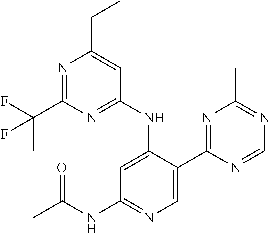 [SMILES: CCc1cc(Nc2cc(NC(C)=O)ncc2-c2ncnc(C)n2)nc(C(C)(F)F)n1] LCMS m/z = 415.1 [M + H]⁺ 1H NMR (500 MHz, DMSO-d₆) δ (ppm) = 12.50 (s, 1H), 10.80 (br s, 1H), 9.37 (d, J = 2.1 Hz, 2H), 9.27 (d, J = 2.1 Hz, 1H), 7.14 (s, 1H), 2.79-2.72 (m, 5H), 2.17-2.08 (m, 6H), 1.29-1.25 (m, 3H). |
| 522 | N-(4′-((2-(1,1-difluoroethyl)-6-methylpyrimidin-4-yl)amino)-5-(2-methoxyethoxy)-[2,3′- bipyridin]-6′-yl)acetamide 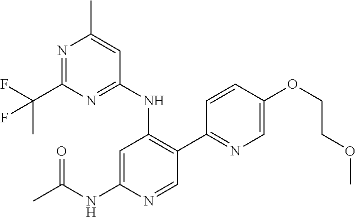 [SMILES: COCCOc1ccc(-c2cnc(NC(C)=O)cc2Nc2cc(C)nc(C(C)(F)F)n2)nc1] LCMS m/z = 459.0 [M + H]⁺; 1H NMR (400 MHz, DMSO-d₆) δ (ppm) = 12.50 (br s, 1H), 10.49 (s, 1H), 9.21 (s, 1H), 8.70 (s, 1H), 8.51 (d, J = 3.0 Hz, 1H), 8.01 (d, J = 9.0 Hz, 1H), 7.60 (dd, J = 3.0, 9.0 Hz, 1H), 7.05 (s, 1H), 4.30-4.24 (m, 2H), 3.74-3.67 (m, 2H), 3.33- 3.30 (m, 3H), 2.42 (s, 3H), 2.16-2.04 (m, 6H). |
| 523 | N-(4′-((2-(1,1-difluoroethyl)pyrimidin-4-yl)amino)-5-(2-methoxyethoxy)-[2,3′-bipyridin]-6′- yl)acetamide 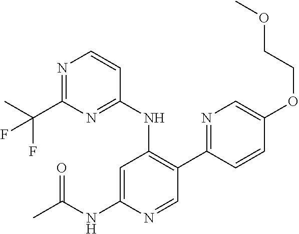 [SMILES: COCCOc1ccc(-c2cnc(NC(C)=O)cc2Nc2ccnc(C(C)(F)F)n2)nc1] LCMS m/z = 445.0 [M + H]⁺; 1H NMR (400 MHz, DMSO-d6) δ (ppm) = 12.47 (s, 1H), 10.52 (s, 1H), 9.19 (s, 1H), 8.71 (s, 1H), 8.55 (d, J = 5.5 Hz, 1H), 8.51 (d, J = 3.0 Hz, 1H), 8.01 (d, J = 9.0 Hz, 1H), 7.61 (dd, J = 3.0, 9.0 Hz, 1H), 7.19 (d, J = 6.0 Hz, 1H), 4.29-4.25 (m, 2H), 3.73-3.69 (m, 2H), 3.32 (s, 3H), 2.14-2.04 (m, 6H). |
| 524 | N-(4′-((2-(1,1-difluoroethyl)-6-methylpyrimidin-4-yl)amino)-5-((difluoromethoxy)methyl)- [2,3′-bipyridin]-6′-yl)acetamide 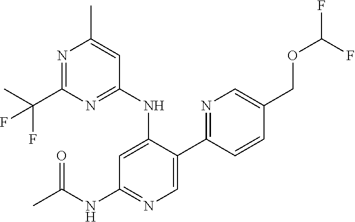 [SMILES: CC(=O)Nc1cc(Nc2cc(C)nc(C(C)(F)F)n2)c(-c2ccc(COC(F)F)cn2)cn1] LCMS m/z = 465 [M + H]⁺ ¹H NMR (CDCl₃, 400 MHz) δ 12.55 (s, 1H), 9.32 (s, 1H), 8.72 (d, 1H, J = 1.8 Hz), 8.59 (s, 1H), 7.88 (dd, 2H, J = 2.0, 8.3 Hz), 7.8-7.9 (m, 1H), 6.78 (s, 1H), 6.39 (t, 1H, J = 73.3 Hz), 5.02 (s, 2H), 2.55 (s, 3H), 2.1-2.3 (m, 6H). |
| 525 | N-(5-cyanO4′-((6-(1,1-difluoroethyl)pyridin-2-yl)amino)-[2,3′-bipyridin]-6′-yl)acetamide 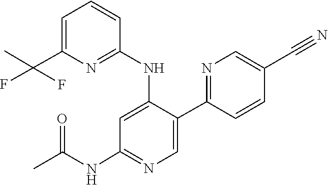 [SMILES: CC(=O)Nc1cc(Nc2cccc(C(C)(F)F)n2)c(-c2ccc(C#N)cn2)cn1] LCMS m/z = 395.1 [M + H]⁺. ¹H NMR (500 MHz, DMSO-d₆) δ (ppm) = 11.67 (s, 1H), 10.56 (s, 1H), 9.25-9.15 (m, 1H), 9.13 (s, 1H), 8.77 (s, 1H), 8.44-8.37 (m, 2H), 8.24 (d, J = 7.9 Hz, 1H), 7.30 (s, 1H), 7.13 (br d, J = 4.9 Hz, 1H), 2.11 (s, 3H), 1.99 (br t, J = 19.1 Hz, 3H). |
| 526 | N-(4-((6-(1,1-difluoroethyl)pyridin-2-yl)amino)-5-(6-methoxypyridazin-3-yl)pyridin-2- yl)acetamide 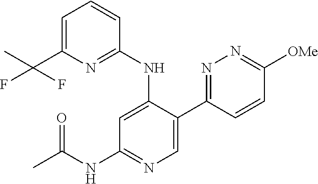 [SMILES: COc1ccc(-c2cnc(NC(C)=O)cc2Nc2cccc(C(C)(F)F)n2)nn1] LCMS m/z = 401.1 [M + H]⁺; 1H NMR (500 MHz, DMSO-d₆) δ (ppm) = 11.47 (s, 1H), 10.53 (br s, 1H), 8.97 (s, 1H), 8.65 (d, J = 2.4 Hz, 1H), 8.44-8.39 (m, 1H), 8.27 (dd, J = 2.4, 9.5 Hz, 1H), 7.40 (dd, J = 2.6, 9.3 Hz, 1H), 7.24 (s, 1H), 7.13 (br d, J = 4.0 Hz, 1H), 4.10 (d, J = 1.8 Hz, 3H), 2.14-2.09 (m, 3H), 1.99 (dt, J = 1.5, 19.2 Hz, 3H). |
| 527 | N-(4-((2-(1,1-difluoroethyl)pyrimidin-4-yl)amino)-5-(6-methoxypyridazin-3-yl)pyridin-2- yl)acetamide-2,2,2-d3 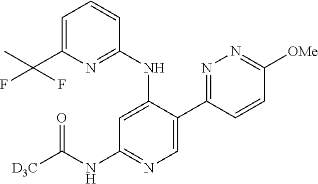 [SMILES: [2H]C([2H])([2H])C(=O)Nc1cc(Nc2cccc(C(C)(F)F)n2)c(-c2ccc(OC)nn2)cn1] LCMS m/z = 405.2 [M + H]+; 1H NMR (400 MHz, DMSO-d6) δ (ppm) = 11.47 (s, 1H), 10.62 (br s, 1H), 9.02 (br s, 1H), 8.67 (s, 1H), 8.57-8.50 (m, 1H), 8.18 (d, J = 9.0 Hz, 1H), 7.38 (d, J = 9.5 Hz, 1H), 7.07 (br d, J = 4.5 Hz, 1H), 4.10 (s, 3H), 2.03 (t, J = 19.0 Hz, 3H). |
| 528 | N-(4-((2-(1,1-difluoroethyl)-6-methylpyrimidin-4-yl)amino)-5-(6,7-dihydrO4H- pyrazolo[5,1-c][1,4]oxazin-2-yl)pyridin-2-yl)acetamide 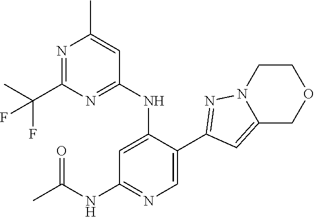 [SMILES: CC(=O)Nc1cc(Nc2cc(C)nc(C(C)(F)F)n2)c(-c2cc3n(n2)CCOC3)cn1] LCMS m/z = 430.1 [M + H]⁺; 1H NMR (400 MHz, DMSO-d₆) δ (ppm) 11.38 (s, 1H), 10.42 (s, 1H), 9.31 (s, 1H), 8.61 (s, 1H), 6.99 (s, 1H), 6.71 (s, 1H), 4.88 (s, 2H), 4.34-4.27 (m, 2H), 4.17-4.11 (m, 2H), 2.44 (s, 3H), 2.19-2.07 (m, 6H). |
| 529 | N-(4-((2-(1,1-difluoroethyl)pyrimidin-4-yl)amino)-5-(6,7-dihydrO4H-pyrazolo[5,1- c][1,4]oxazin-2-yl)pyridin-2-yl)acetamide 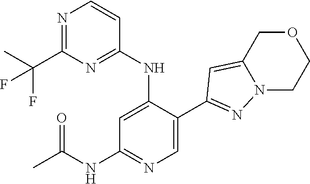 [SMILES: CC(=O)Nc1cc(Nc2ccnc(C(C)(F)F)n2)c(-c2cc3n(n2)CCOC3)cn1] LCMS m/z = 416.0 [M + H]⁺ 1H NMR (400 MHz, DMSO-d₆) δ (ppm): 11.43 (s, 1H), 10.46 (s, 1H), 9.30 (s, 1H), 8.64 (s, 1H), 8.59 (d, J = 5.5 Hz, 1H), 7.18 (d, J = 5.5 Hz, 1H), 6.73 (s, 1H), 4.88 (s, 2H), 4.30 (t, J = 5.3 Hz, 2H), 4.18-4.09 (m, 2H), 2.19-2.08 (m, 6H). |
| 530 | N-(5-(6,7-dihydrO4H-pyrazolo[5,1-c][1,4]oxazin-2-yl)-4-((2-(2-fluoropropan-2-yl)-6- methylpyrimidin-4-yl)amino)pyridin-2-yl)acetamide 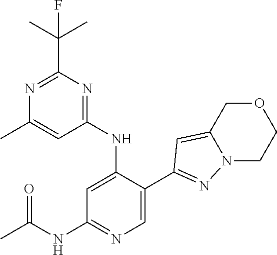 [SMILES: CC(=O)Nc1cc(Nc2cc(C)nc(C(C)(C)F)n2)c(-c2cc3n(n2)CCOC3)cn1] LCMS m/z = 426.1 [M + H]⁺; 1H NMR (400 MHz, DMSO-d₆) δ (ppm): 11.26 (s, 1H), 10.39 (s, 1H), 9.37 (s, 1H), 8.61 (s, 1H), 6.87 (s, 1H), 6.73 (s, 1H), 4.89 (s, 2H), 4.32 (t, J = 5.0 Hz, 2H), 4.16-4.13 (m, 2H), 2.41 (s, 3H), 2.10 (s, 3H), 1.79 (s, 3H), 1.74 (s, 3H). |
| 531 | N-(5-(5,6-dihydrO8H-imidazo[2,1-c][1,4]oxazin-2-yl)-4-((2-(2-fluoropropan-2-yl)-6- methylpyrimidin-4-yl)amino)pyridin-2-yl)acetamide 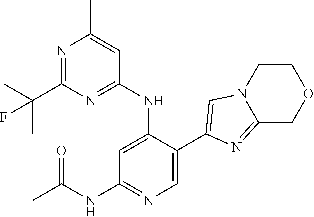 [SMILES: CC(=O)Nc1cc(Nc2cc(C)nc(C(C)(C)F)n2)c(-c2cn3c(n2)COCC3)cn1] LCMS m/z = 426.2 [M + H]⁺; 1H NMR (500 MHz, CDCl₃) δ (ppm): 11.96 (s, 1H), 9.40 (br s, 1H), 8.35 (s, 1H), 7.97 (br s, 1H), 7.26 (s, 1H), 6.56 (s, 1H), 4.93 (s, 2H), 4.11 (s, 4H), 2.45 (s, 3H), 2.18 (s, 3H), 1.84 (s, 3H), 1.80 (s, 3H). |
| 532 | N-(5-([1,2,4]triazolo[4,3-a]pyrazin-6-yl)-4-((2-(2-fluoropropan-2-yl)-6-methylpyrimidin-4- yl)amino)pyridin-2-yl)acetamide 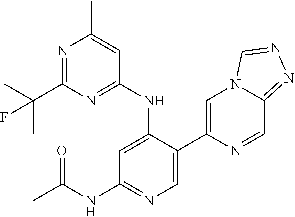 [SMILES: CC(=O)Nc1cc(Nc2cc(C)nc(C(C)(C)F)n2)c(-c2cn3cnnc3cn2)cn1] LCMS m/z = 422.2 [M + H]⁺ 1H NMR (500 MHz, DMSO-d₆) δ (ppm): 10.53 (s, 1H), 9.60 (s, 1H), 9.53 (s, 1H), 9.49 (s, 1H), 9.11 (s, 1H), 8.94 (s, 1H), 8.39 (s, 1H), 6.75 (s, 1H), 2.34 (s, 3H), 2.12 (s, 3H), 1.71 (s, 3H), 1.67 (s, 3H). |
| 533 | N-(4-((2-(2-fluoropropan-2-yl)-6-methylpyrimidin-4-yl)amino)-5-(imidazo[1,2-a]pyrimidin- 7-yl)pyridin-2-yl)acetamide 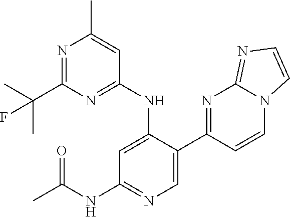 [SMILES: CC(=O)Nc1cc(Nc2cc(C)nc(C(C)(C)F)n2)c(-c2ccn3ccnc3n2)cn1] LCMS m/z = 421.2 [M + H]⁺; 1H NMR (500 MHz, DMSO-d₆) δ (ppm): 12.32 (s, 1H), 10.63 (s, 1H), 9.32 (s, 1H), 9.06 (d, J = 7.0 Hz, 1H), 8.91 (s, 1H), 7.97 (d, J = 1.2 Hz, 1H), 7.79 (d, J = 1.2 Hz, 1H), 7.74 (d, J = 7.3 Hz, 1H), 6.75 (s, 1H), 2.44 (s, 3H), 2.15 (s, 3H), 1.76 (s, 3H), 1.72 (s, 3H). |
| 534 | N-(5-([1,2,4]triazolo[1,5-a]pyrimidin-5-yl)-4-((2-(2-fluoropropan-2-yl)-6-methylpyrimidin- 4-yl)amino)pyridin-2-yl)acetamide 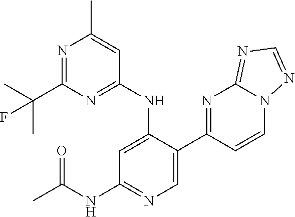 [SMILES: CC(=O)Nc1cc(Nc2cc(C)nc(C(C)(C)F)n2)c(-c2ccn3ncnc3n2)cn1] LCMS m/z = 422.2 [M + H]⁺; 1H NMR (500 MHz, DMSO-d6) δ (ppm) = 11.41 (s, 1H), 10.69 (br s, 1H), 9.44 (br d, J = 7.3 Hz, 1H), 9.19 (s, 1H), 8.88 (s, 1H), 8.70 (s, 1H), 7.89 (br d, J = 7.0 Hz, 1H), 6.74 (s, 1H), 2.40 (s, 3H), 2.14 (s, 3H), 1.70 (s, 3H), 1.66 (s, 3H). |
| 535 | N-(4-((2-(1,1-difluoroethyl)-6-methylpyrimidin-4-yl)amino)-5-(6,7-dihydrO5H- pyrazolo[5,1-b][1,3]oxazin-3-yl)pyridin-2-yl)acetamide 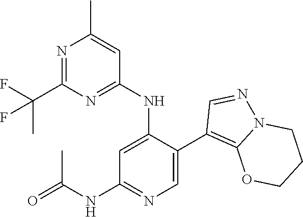 [SMILES: CC(=O)Nc1cc(Nc2cc(C)nc(C(C)(F)F)n2)c(-c2cnn3c2OCCC3)cn1] LCMS m/z = 430.2 [M + H]⁺ 1H NMR (500 MHz, DCM-d₂) δ (ppm) = 8.79 (s, 1H), 8.14 (s, 1H), 8.10-8.02 (m, 1H), 7.44 (s, 1H), 7.42 (s, 1H), 6.78 (s, 1H), 4.40-4.37 (m, 2H), 4.23 (t, J = 6.3 Hz, 2H), 2.47 (s, 3H), 2.35-2.30 (m, 2H), 2.19 (s, 3H), 2.05 (t, J = 18.9 Hz, 3H). |
| 536 | N-(5-([1,2,4]triazolo[1,5-a]pyrimidin-5-yl)-4-((2-(1,1-difluoroethyl)-6-methylpyrimidin-4- yl)amino)pyridin-2-yl)acetamide 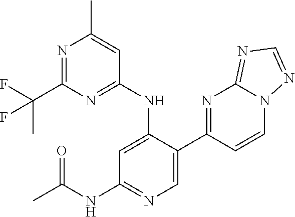 [SMILES: CC(=O)Nc1cc(Nc2cc(C)nc(C(C)(F)F)n2)c(-c2ccn3ncnc3n2)cn1] LCMS m/z = 426.1 [M + H]⁺; 1H NMR (500 MHz, DMSO-d₆) δ (ppm) = 11.32 (s, 1H), 10.74 (br s, 1H), 9.42 (br d, J = 7.0 Hz, 1H), 9.05 (br s, 1H), 8.85 (s, 1H), 8.70 (s, 1H), 7.84 (br d, J = 6.7 Hz, 1H), 6.88 (br s, 1H), 2.43 (s, 3H), 2.15 (s, 3H), 1.99 (t, J = 19.2 Hz, 3H). |
| 537 | N-(5-([1,2,4]triazolo[1,5-a]pyrimidin-5-yl)-4-((2-(1,1-difluoroethyl)pyrimidin-4- yl)amino)pyridin-2-yl)acetamide 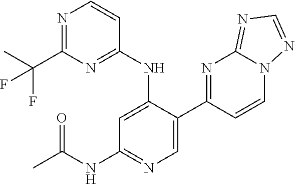 [SMILES: CC(=O)Nc1cc(Nc2ccnc(C(C)(F)F)n2)c(-c2ccn3ncnc3n2)cn1] LCMS m/z = 412.1 [M + H]⁺; 1H NMR (500 MHz, DMSO-d₆) δ (ppm) = 11.31 (s, 1H), 10.76 (br s, 1H), 9.41 (br s, 1H), 9.07-8.91 (m, 1H), 8.84 (s, 1H), 8.68 (br s, 1H), 8.61-8.51 (m, 1H), 7.82 (br s, 1H), 7.02 (br s, 1H), 2.14 (s, 3H), 1.97 (br t, J = 19.1 Hz, 3H). |
| 538 | N-(4-((2-(2-fluoropropan-2-yl)-6-methylpyrimidin-4-yl)amino)-5-(pyrazolo[1,5-a]pyrimidin- 5-yl)pyridin-2-yl)acetamide 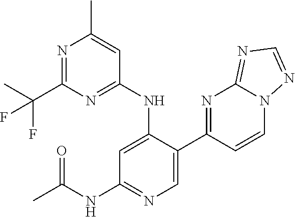 LCMS m/z = 421.2 [M + H]+; 1H NMR (500 MHz, DMSO-d₆) δ (ppm) = 12.17 (s, 1H), 10.63 (s, 1H), 9.32 (s, 1H), 9.19 (d, J = 7.6 Hz, 1H), 8.92 (s, 1H), 8.28 (d, J = 2.1 Hz, 1H), 7.67 (d, J = 7.6 Hz, 1H), 6.98 (d, J = 2.1 Hz, 1H), 6.92 (s, 1H), 2.43 (s, 3H), 2.14 (s, 3H), 1.75 (s, 3H), 1.70 (s, 3H). |
| 539 | N-(4-((2-(1,1-difluoroethyl)pyrimidin-4-yl)amino)-5-(pyrazolo[1,5-a]pyrimidin-5-yl)pyridin- 2-yl)acetamide 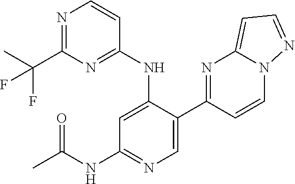 [SMILES: CC(=O)Nc1cc(Nc2ccnc(C(C)(F)F)n2)c(-c2ccn3nccc3n2)cn1] LCMS m/z = 411.1 [M + H]⁺; 1H NMR (500 MHz, DMSO-d6) δ (ppm) = 12.12 (s, 1H), 10.70 (s, 1H), 9.24-9.15 (m, 2H), 8.91 (s, 1H), 8.59 (br d, J = 5.5 Hz, 1H), 8.27 (d, J = 1.5 Hz, 1H), 7.63 (br d, J = 7.6 Hz, 1H), 7.22 (br d, J = 5.5 Hz, 1H), 6.92 (s, 1H), 2.14 (s, 3H), 2.06 (br t, J = 19.2 Hz, 3H). |
| 540 | N-(4-((2-(1,1-difluoroethyl)-6-methylpyrimidin-4-yl)amino)-5-(pyrazolo[1,5-a]pyrimidin-5- yl)pyridin-2-yl)acetamide 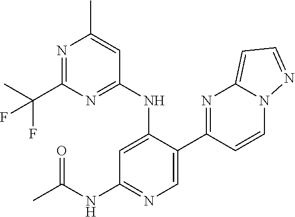 [SMILES: CC(=O)Nc1cc(Nc2cc(C)nc(C(C)(F)F)n2)c(-c2ccn3nccc3n2)cn1] LCMS m/z = 425.2 [M + H]⁺; 1H NMR (500 MHz, DMSO-d₆) δ (ppm) = 12.17 (s, 1H), 10.67 (br s, 1H), 9.24 (br s, 1H), 9.18 (br d, J = 7.3 Hz, 1H), 8.91 (br s, 1H), 8.28 (br s, 1H), 7.64 (br d, J = 7.3 Hz, 1H), 7.06 (br s, 1H), 6.98 (br s, 1H), 2.54 (s, 3H), 2.14 (s, 3H), 2.06 (br t, J = 19.1 Hz, 3H). |
| 541 | N-(4-((2-(1,1-difluoroethyl)-6-methylpyrimidin-4-yl)amino)-5-(5-methyl-4-oxO4,5,6,7- tetrahydropyrazolo[1,5-a]pyrazin-2-yl)pyridin-2-yl)acetamide 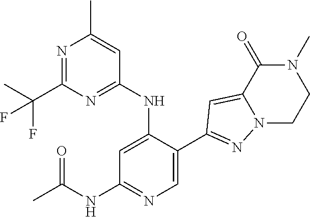 [SMILES: CC(=O)Nc1cc(Nc2cc(C)nc(C(C)(F)F)n2)c(-c2cc3n(n2)CCN(C)C3=O)cn1] LCMS m/z = 457.1 [M + H]⁺; 1H NMR (500 MHz, DMSO-d₆) δ (ppm) = 11.06 (s, 1H), 10.48 (s, 1H), 9.28 (s, 1H), 8.76 (s, 1H), 7.42 (s, 1H), 7.09 (s, 1H), 4.59 (br t, J = 6.0 Hz, 2H), 3.88 (br t, J = 5.8 Hz, 2H), 3.05 (s, 3H), 2.45 (s, 3H), 2.11 (s, 6H). |
| 542 | N-(4-((2-(1,1-difluoroethyl)-6-methylpyrimidin-4-yl)amino)-5-(1-(ethylsulfonyl)-1H- pyrazol-3-yl)pyridin-2-yl)acetamide 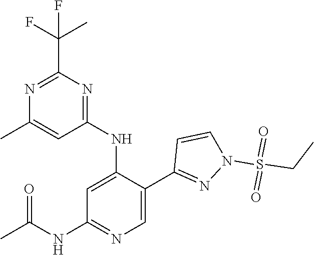 [SMILES: CCS(=O)(=O)n1ccc(-c2cnc(NC(C)=O)cc2Nc2cc(C)nc(C(C)(F)F)n2)n1] LCMS m/z = 466.1 [M + H]⁺; 1H NMR (500 MHz, DMSO-d6) δ (ppm) = 10.74 (s, 1H), 10.61 (s, 1H), 9.13 (s, 1H), 8.78 (s, 1H), 8.48 (d, J = 2.7 Hz, 1H), 7.28 (d, J = 3.1 Hz, 1H), 6.82 (s, 1H), 3.83 (q, J = 7.3 Hz, 2H), 2.44 (s, 3H), 2.12 (s, 3H), 2.06 (t, J = 19.1 Hz, 3H), 1.14 (t, J = 7.2 Hz, 3H). |
| 543 | N-(4′-((2-(1,1-difluoroethyl)-6-methylpyrimidin-4-yl)amino)-5-(methylsulfonyl)-[2,3′- bipyridin]-6′-yl)acetamide 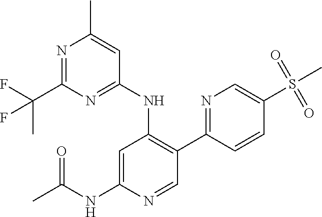 [SMILES: CC(=O)Nc1cc(Nc2cc(C)nc(C(C)(F)F)n2)c(-c2ccc(S(C)(=O)=O)cn2)cn1] LCMS m/z = 463.1 [M + H]⁺ 1H NMR (500 MHz, DMSO-d₆) δ (ppm): 12.26 (s, 1H), 10.65 (s, 1H), 9.27 (s, 2H), 8.86 (s, 1H), 8.43 (dd, J = 2.4, 8.9 Hz, 1H), 8.32 (d, J = 8.2 Hz, 1H), 7.19 (s, 1H), 3.38 (s, 3H), 2.43 (s, 3H), 2.14 (s, 3H), 2.08 (t, J = 19.2 Hz, 3H). |
| 544 | N-(4-((2-(2-oxabicyclo[2.1.1]hexan-4-yl)-6-methylpyrimidin-4-yl)amino)-5-(5-ethyl-4,5,6,7- tetrahydropyrazolo[1,5-a]pyrazin-2-yl)pyridin-2-yl)acetamide 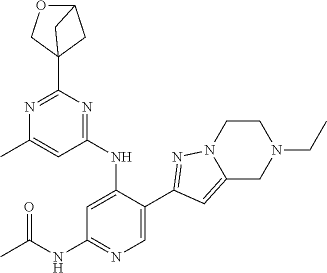 [SMILES: CCN1CCn2nc(-c3cnc(NC(C)=O)cc3Nc3cc(C)nc(C45COC(C4)C5)n3)cc2C1] LCMS m/z = 475.3 [M + H]+; 1H NMR (400 MHz, DMSO-d6) δ (ppm) = 11.36 (s, 1H), 10.36 (s, 1H), 9.52 (s, 1H), 8.58 (s, 1H), 6.78 (s, 1H), 6.69 (s, 1H), 4.58-4.53 (m, 1H), 4.29 (t, J = 5.5 Hz, 2H), 3.98 (s, 2H), 3.71 (s, 2H), 2.96 (t, J = 5.5 Hz, 2H), 2.60 (q, J = 7.2 Hz, 2H), 2.39-2.36 (m, 5H), 2.11 (s, 3H), 1.84 (dd, J = 1.8, 4.3 Hz, 2H), 1.11 (t, J = 7.0 Hz, 3H). |
| 545 | N-(4-((2-(1,1-difluoroethyl)-6-methylpyrimidin-4-yl)amino)-5-(5-(methyl-d3)-4,5,6,7- tetrahydropyrazolo[1,5-a]pyrazin-2-yl)pyridin-2-yl)acetamide 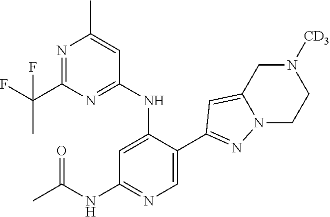 [SMILES: [2H]C([2H])([2H])N1CCn2nc(-c3cnc(NC(C)=O)cc3Nc3cc(C)nc(C(C)(F)F)n3)cc2C1] LCMS m/z = 446.2 [M + H]⁺; 1H NMR (500 MHz, DMSO-d6) δ (ppm) = 11.49 (s, 1H), 10.43 (s, 1H), 9.33 (s, 1H), 8.62 (s, 1H), 7.03 (s, 1H), 6.71 (s, 1H), 4.30 (s, 2H), 3.66 (s, 2H), 2.91 (s, 2H), 2.45 (s, 3H), 2.17-2.09 (m, 6H). |
| 546 | N-(4-((2-(1,1-difluoroethyl)-6-methylpyrimidin-4-yl)amino)-5-(6-(difluoromethyl)pyridazin- 3-yl)pyridin-2-yl)acetamide 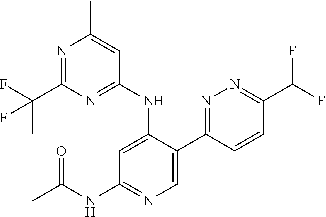 [SMILES: CC(=O)Nc1cc(Nc2cc(C)nc(C(C)(F)F)n2)c(-c2ccc(C(F)F)nn2)cn1] LCMS m/z = 436.1 [M + H]⁺; 1H NMR (400 MHz, DCM-d₂) δ (ppm): 12.37 (br s, 1H), 9.53 (br s, 1H), 8.61 (br s, 1H), 8.51-8.30 (m, 1H), 8.17 (d, J = 9.0 Hz, 1H), 7.98 (d, J = 9.0 Hz, 1H), 7.20-6.79 (m, 2H), 2.52 (s, 3H), 2.25 (s, 3H), 2.21-2.08 (m, 3H). |
| 547 | N-(4-((6-methyl-2-(tetrahydrofuran-3-yl)pyrimidin-4-yl)amino)-5-(5-methyl-4,5,6,7- tetrahydropyrazolo[1,5-a]pyrazin-2-yl)pyridin-2-yl)acetamide 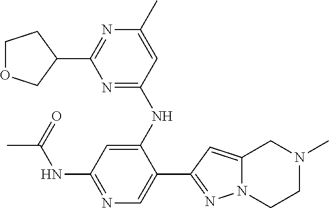 [SMILES: CC(=O)Nc1cc(Nc2cc(C)nc(C3CCOC3)n2)c(-c2cc3n(n2)CCN(C)C3)cn1] LCMS m/z = 449.2 [M + H]⁺; 1H NMR (500 MHz, DCM-d₂) δ (ppm): 12.41 (s, 1H), 12.18 (br s, 1H), 9.88 (s, 1H), 8.50 (s, 1H), 6.89 (s, 1H), 6.70 (s, 1H), 4.73 (br s, 2H), 4.67-4.48 (m, 2H), 4.27-4.22 (m, 1H), 4.18 (dt, J = 5.2, 8.2 Hz, 1H), 4.11 (t, J = 7.6 Hz, 1H), 3.95 (q, J = 7.5 Hz, 2H), 3.90-3.76 (m, 2H), 3.10 (s, 3H), 2.69 (s, 3H), 2.67-2.59 (m, 1H), 2.53- 2.46 (m, 1H), 2.33 (s, 3H). |
| 548 | N-(4-((2-(1,1-difluoroethyl)-6-methylpyrimidin-4-yl)amino)-5-(5,6-dimethyl-4,5,6,7- tetrahydropyrazolo[1,5-a]pyrazin-2-yl)pyridin-2-yl)acetamide 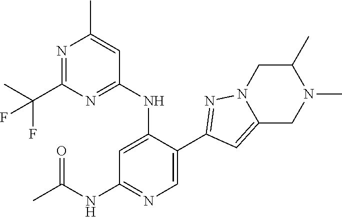 [SMILES: CC(=O)Nc1cc(Nc2cc(C)nc(C(C)(F)F)n2)c(-c2cc3n(n2)CC(C)N(C)C3)cn1] LCMS m/z = 457.4 [M + H]⁺ 1H NMR (400 MHz, CDCl₃) δ ppm 11.37 (s, 1H), 9.35 (s, 1 H), 8.46 (s, 1H), 7.87 (s, 1H), 6.74 (s, 1H), 6.39 (s, 1H), 4.28 (dd, J = 12.5, 4.0 Hz, 1H), 4.01 (d, J = 15.5 Hz, 1H), 3.96 (dd, J = 12.3, 8.8 Hz, 1H), 3.63 (d, J = 15.5 Hz, 2H), 3.50 (s, 2H), 2.97 (ddd, J = 9.0, 6.5, 4.0 Hz, 1H), 2.55 (s, 3H), 2.48 (s, 3H), 2.24 (s, 3H), 2.24 (t, J = 18.0 Hz, 3H), 1.30 (d, J = 6.5 Hz, 3H). |
| 549 | N-(4-((2-(2-methoxyethoxy)pyrimidin-4-yl)amino)-5-(1-methyl-1H-pyrazol-3-yl)pyridin-2- yl)acetamide 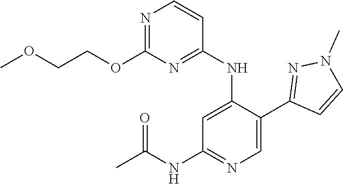 [SMILES: COCCOc1nccc(Nc2cc(NC(C)=O)ncc2-c2ccn(C)n2)n1] LCMS m/z = 384 [M + H]⁺ 1H NMR (MeOH-d4, 400 MHz) 9.2-9.4 (m, 1H), 8.50 (s, 1H), 8.13 (d, 1H, J = 5.8 Hz), 7.66 (d, 1H, J = 2.3 Hz), 6.73 (d, 1H, J = 2.5 Hz), 6.54 (d, 1H, J = 5.8 Hz), 4.66 (dd, 2H, J = 3.8, 5.3 Hz), 4.00 (s, 3H), 3.78-3.80 (m, 2H), 3.43 (s, 3H), 2.17 (s, 3H). |
| 550 | N-(4-((4-(2-methoxyethoxy)pyrimidin-2-yl)amino)-5-(1-methyl-1H-pyrazol-3-yl)pyridin-2- yl)acetamide 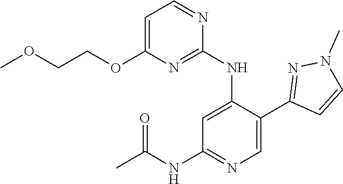 [SMILES: COCCOc1ccnc(Nc2cc(NC(C)=O)ncc2-c2ccn(C)n2)n1] LCMS m/z = 384 [M + H]⁺ 1H NMR (DMSO-d₆, 400 MHz) δ 11.50 (s, 1H), 10.37 (s, 1H), 9.38 (s, 1H), 8.62 (s, 1H), 8.32 (d, 1H, J = 5.8 Hz), 7.88 (d, 1H, J = 2.3 Hz), 6.92 (d, 1H, J = 2.5 Hz), 6.48 (d, 1H, J = 5.8 Hz), 4.6-4.7 (m, 2H), 3.97 (s, 3H), 3.7-3.7 (m, 2H), 3.32 (s, 3H). |
| 551 | N-(4-((2-methoxy-6-methylpyrimidin-4-yl)amino)-5-(1-methyl-1H-pyrazol-3-yl)pyridin-2- yl)acetamide 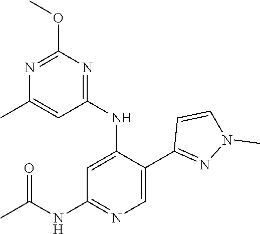 [SMILES: COc1nc(C)cc(Nc2cc(NC(C)=O)ncc2-c2ccn(C)n2)n1] LCMS m/z = 354 [M + H]⁺ 1H NMR (DMSO-d₆, 600 MHz) δ 11.4-11.6 (m, 1H), 10.55 (br s, 1H), 9.29 (s, 1H), 8.65 (s, 1H), 7.90 (d, 1H, J = 2.4 Hz), 6.92 (d, 1H, J = 2.4 Hz), 6.63 (s, 1H), 4.03 (s, 3H), 4.01 (s, 3H), 2.34 (s, 3H), 2.11 (s, 3H). |
| 552 | N-(4-((2-(2-oxabicyclo[2.1.1]hexan-4-yl)-6-methylpyrimidin-4-yl)amino)-5-(1-methyl-1H- pyrazol-3-yl)pyridin-2-yl)acetamide 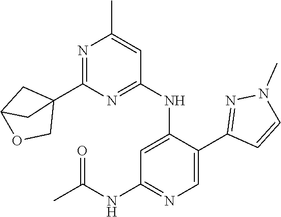 [SMILES: CC(=O)Nc1cc(Nc2cc(C)nc(C34COC(C3)C4)n2)c(-c2ccn(C)n2)cn1] LCMS m/z = 406 [M + H]⁺ 1H NMR (DMSO-d₆, 600 MHz) δ 11.3-11.5 (m, 1H), 10.38 (s, 1H), 9.51 (s, 1H), 8.63 (s, 1H), 7.89 (d, 1H, J = 2.3 Hz), 6.92 (d, 1H, J = 2.4 Hz), 6.82 (s, 1H), 4.56 (s, 1H), 4.03 (s, 3H), 3.98 (s, 2H), 2.3-2.4 (m, 5H), 2.11 (s, 3H), 1.84 (dd, 2H, J = 1.6, 4.4 Hz). |
| 553 | N-(4-((4-methoxypyrimidin-2-yl)amino)-5-(1-methyl-1H-pyrazol-3-yl)pyridin-2- yl)acetamide 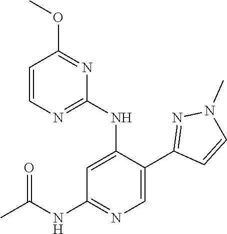 [SMILES: COc1ccnc(Nc2cc(NC(C)=O)ncc2-c2ccn(C)n2)n1] LCMS m/z = 340 [M + H]+ 1H NMR (DMSO-d6, 600 MHz) δ 11.5-11.8 (m, 1H), 10.4- 10.8 (m, 1H), 9.36 (s, 1H), 8.61 (s, 1H), 8.3-8.4 (m, 1H), 7.90 (d, 1H, J = 2.3 Hz), 6.92 (d, 1H, J = 2.4 Hz), 6.50 (d, 1H, J = 5.6 Hz), 4.07 (s, 3H), 3.98 (s, 3H), 2.11 (s, 3H) |
| 554 | N-(5-(1-methyl-1H-pyrazol-3-yl)-4-((6-methyl-2-(1-methyl-2-oxabicyclo[2.1.1]hexan-4- yl)pyrimidin-4-yl)amino)pyridin-2-yl)acetamide 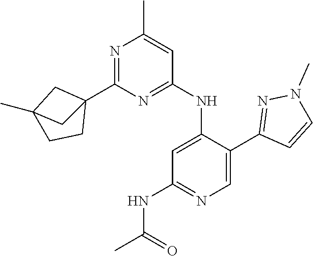 [SMILES: CC(=O)Nc1cc(Nc2cc(C)nc(C34CCC(C)(C3)C4)n2)c(-c2ccn(C)n2)cn1] LCMS m/z = 420 [M + H]⁺ 1H NMR (DMSO-d6, 400 MHz) δ 11.3-11.5 (m, 1H), 10.37 (s, 1H), 9.59 (s, 1H), 8.63 (s, 1H), 7.88 (d, 1H, J = 2.0 Hz), 6.92 (d, 1H, J = 2.0 Hz), 6.80 (s, 1H), 3.9-4.1 (m, 5H), 2.37 (s, 5H), 2.12 (s, 3H), 1.7-1.8 (m, 2H), 1.44 (s, 3H). |
| 555 | N-(5-(1-methyl-1H-pyrazol-3-yl)-4-((6-methyl-2-(tetrahydrofuran-3-yl)pyrimidin-4- yl)amino)pyridin-2-yl)acetamide 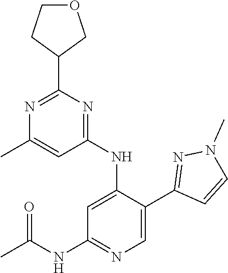 [SMILES: CC(=O)Nc1cc(Nc2cc(C)nc(C3CCOC3)n2)c(-c2ccn(C)n2)cn1] LCMS m/z = 394 [M + H]⁺ 1H NMR (DMSO-d6, 600 MHz) δ 11.25 (s, 1H), 10.36 (s, 1H), 9.43 (s, 1H), 8.63 (s, 1H), 7.88 (d, 1H, J = 2.2 Hz), 6.91 (d, 1H, J = 2.4 Hz), 6.80 (s, 1H), 4.14 (t, 1H, J = 8.1 Hz), 4.02 (s, 3H), 3.7-3.9 (m, 3H), 3.55 (quin, 1H, J = 7.9 Hz), 2.43 (qd, 1H, J = 7.6, 12.2 Hz), 2.37 (s, 3H), 2.26 (dddd, 1H, J = 5.5, 7.3, 8.6, 12.4 Hz), 2.10 (s, 3H). |
| 556 | N-(5-(1-methyl-1H-pyrazol-3-yl)-4-((2-(tetrahydrofuran-3-yl)pyrimidin-4-yl)amino)pyridin- 2-yl)acetamide 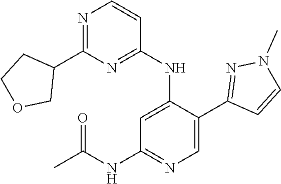 [SMILES: CC(=O)Nc1cc(Nc2ccnc(C3CCOC3)n2)c(-c2ccn(C)n2)cn1] LCMS m/z = 380 [M + H]⁺ 1H NMR (DMSO-d6, 600 MHz) δ 11.4-11.6 (m, 1H), 10.60 (s, 1H), 9.29 (s, 1H), 8.67 (s, 1H), 8.47 (d, 1H, J = 6.0 Hz), 7.89 (d, 1H, J = 2.3 Hz), 7.04 (d, 1H, J = 6.1 Hz), 6.90 (d, 1H, J = 2.3 Hz), 4.14 (t, 1H, J = 8.1 Hz), 4.00 (s, 3H), 3.8-3.9 (m, 2H), 3.8-3.8 (m, 1H), 3.6-3.7 (m, 1H), 2.4-2.4 (m, 1H), 2.29 (dddd, 1H, J = 5.6, 7.2, 8.8, 12.5 Hz), 2.13 (s, 3H). |
| 557 | N-(4-((2-(2-oxabicyclo[2.2.1]heptan-4-yl)-6-methylpyrimidin-4-yl)amino)-5-(1-methyl-1H- pyrazol-3-yl)pyridin-2-yl)acetamide 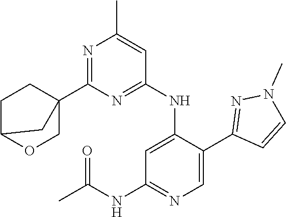 [SMILES: CC(=O)Nc1cc(Nc2cc(C)nc(C34CCC(C3)OC4)n2)c(-c2ccn(C)n2)cn1] LCMS m/z = 420 [M + H]⁺ 1H NMR (CDCl₃, 400 MHz) δ 11.5-11.7 (m, 1H), 10.5-10.9 (m, 1H), 9.76 (s, 1H), 8.30 (s, 1H), 7.51 (d, 1H, J = 2.3 Hz), 6.62 (d, 2H, J = 2.8 Hz), 4.5-4.6 (m, 1H), 4.1-4.2 (m, 1H), 4.1-4.1 (m, 1H), 4.07 (s, 3H), 2.51 (s, 3H), 2.3-2.3 (m, 5H), 2.1-2.2 (m, 2H), 1.9-2.0 (m, 2H). |
| 558 | N-(4-((2-(1-fluorocyclopropyl)-6-methylpyrimidin-4-yl)amino)-5-(1-methyl-1H-pyrazol-3- yl)pyridin-2-yl)acetamide 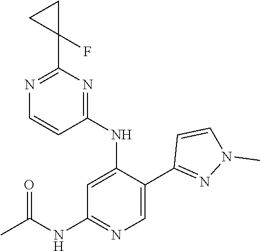 LCMS m/z = 382 [M + H]+; 1H NMR (DMSO-d6, 600 MHz) δ 11.33 (s, 1H), 10.42 (s, 1H), 9.06 (s, 1H), 8.63 (s, 1H), 7.88 (d, 1H, J = 2.3 Hz), 6.91 (d, 1H, J = 2.4 Hz), 6.83 (s, 1H), 4.02 (s, 3H), 2.41 (s, 3H), 2.10 (s, 3H), 1.6-1.7 (m, 2H), 1.4-1.5 (m, 2H). |
| 559 | N-(4-((2-(2-methoxyethoxy)-6-methylpyrimidin-4-yl)amino)-5-(1-methyl-1H-pyrazol-3- yl)pyridin-2-yl)acetamide 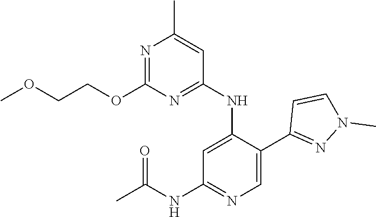 LCMS m/z = 398 [M + H]⁺ |
| 560 | N-(4-((2-((1s,4s)-1-(fluoromethyl)-2-oxabicyclo[2.1.1]hexan-4-yl)-6-methylpyrimidin-4- yl)amino)-5-(1-methyl-1H-pyrazol-3-yl)pyridin-2-yl)acetamide 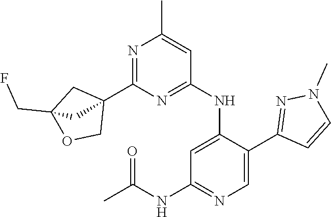 [SMILES: CC(=O)Nc1cc(Nc2cc(C)nc([C@]34CO[C@](CF)(C3)C4)n2)c(-c2ccn(C)n2)cn1] LCMS m/z = 438 [M + H]⁺ 1H NMR (DMSO-d6, 600 MHz) δ 11.4-11.6 (m, 1H), 10.44 (s, 1H), 9.58 (br s, 1H), 8.64 (s, 1H), 7.89 (d, 1H, J = 1.7 Hz), 6.93 (d, 1H, J = 1.8 Hz), 6.85 (s, 1H), 4.6-4.8 (m, 2H), 4.11 (s, 2H), 4.04 (s, 3H), 2.5-2.5 (m, 2H), 2.40 (s, 3H), 2.13 (s, 3H), 1.91 (br d, 2H, J = 4.1 Hz). |
| 561 | N-(4-((2-(1-(methoxymethyl)-2-oxabicyclo[2.1.1]hexan-4-yl)-6-methylpyrimidin-4- yl)amino)-5-(1-methyl-1H-pyrazol-3-yl)pyridin-2-yl)acetamide 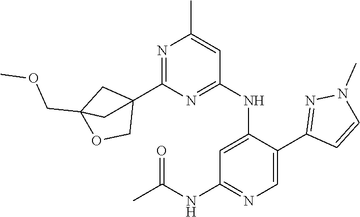 [SMILES: COCC12CC(c3nc(C)cc(Nc4cc(NC(C)=O)ncc4-c4ccn(C)n4)n3)(CO1)C2] LCMS m/z = 450 [M + H]⁺ 1H NMR (DMSO-d6, 600 MHz) δ 11.4-11.5 (m, 1H), 10.42 (s, 1H), 9.53 (br s, 1H), 8.5-8.7 (m, 1H), 7.89 (d, 1H, J = 2.3 Hz), 6.92 (d, 1H, J = 2.4 Hz), 6.8- 6.9 (m, 1H), 4.04 (d, 5H, J = 15.7 Hz), 3.6-3.7 (m, 3H), 3.3-3.4 (m, 2H), 2.4-2.4 (m, 5H), 2.12 (s, 3H), 1.85 (dd, 2H, J = 1.5, 4.1 Hz). |
| 562 | N-(4-((2-(2-oxabicyclo[2.1.1]hexan-1-yl)-6-methylpyrimidin-4-yl)amino)-5-(1-methyl-1H- pyrazol-3-yl)pyridin-2-yl)acetamide 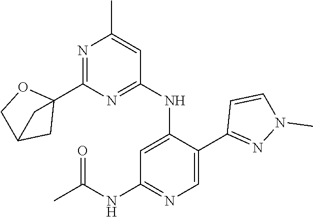 [SMILES: CC(=O)Nc1cc(Nc2cc(C)nc(C34CC(CO3)C4)n2)c(-c2ccn(C)n2)cn1] LCMS m/z = 406 [M + H]⁺; 1H NMR (MeOH-d4, 400 MHz) δ 9.4-9.5 (m, 1H), 8.54 (s, 1H), 7.69 (d, 1H, J = 2.3 Hz), 6.79-6.80 (m, 1H), 6.76 (d, 1H, J = 2.5 Hz), 4.05 (s, 3H), 3.98 (s, 2H), 3.06 (t, 1H, J = 3.3 Hz), 2.62 (ddd, 2H, J = 1.9, 3.1, 4.6 Hz), 2.46 (s, 3H), 2.21 (s, 3H), 1.8-1.9 (m, 2H). |
| 563 | N-(4-((2-(2-oxabicyclo[2.1.1]hexan-4-yl)-6-methylpyrimidin-4-yl)amino)-5-(2,2-dimethyl- 2,3-dihydrO[1,4]dioxino[2,3-b]pyridin-6-yl)pyridin-2-yl)acetamide 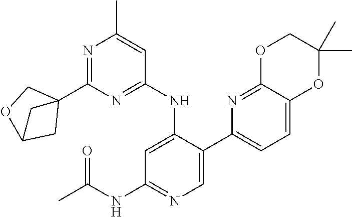 [SMILES: CC(=O)Nc1cc(Nc2cc(C)nc(C34COC(C3)C4)n2)c(-c2ccc3c(n2)OCC(C)(C)O3)cn1] LCMS m/z = 489 [M + H]⁺ 1H NMR (CDCl₃, 400 MHz) δ 12.15 (s, 1H), 9.49 (s, 1H), 8.47 (s, 1H), 8.0-8.2 (m, 1H), 7.35-7.37 (m, 1H), 7.29-7.31 (m, 1H), 6.57 (s, 1H), 4.67 (t, 1H, J = 1.0 Hz), 4.18 (d, 4H, J = 8.5 Hz), 2.4-2.5 (m, 5H), 2.23 (s, 3H), 2.0-2.1 (m, 2H), 1.45 (s, 6H). |
| 564 | N-(4-((2-(2-oxabicyclo[2.1.1]hexan-4-yl)-6-methylpyrimidin-4-yl)amino)-5-(5,5- difluorO5,6-dihydrO4H-pyrrolo[1,2-b]pyrazol-2-yl)pyridin-2-yl)acetamide 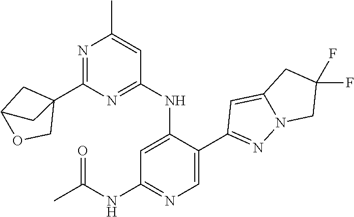 [SMILES: CC(=O)Nc1cc(Nc2cc(C)nc(C34COC(C3)C4)n2)c(-c2cc3n(n2)CC(F)(F)C3)cn1] LCMS m/z = 468 [M + H]+ 1H NMR (CDCl₃, 400 MHz) d 10.7-10.9 (m, 1H), 9.50 (s, 1H), 8.44 (s, 1H), 7.89 (br s, 1H), 6.56 (s, 1H), 6.50 (s, 1H), 4.6-4.8 (m, 3H), 4.17 (s, 2H), 3.57 (t, 2H, J = 13.5 Hz), 2.4-2.5 (m, 5H), 2.23 (s, 3H), 2.0-2.1 (m, 2H). |
| 565 | N-(4′-((2-(2-oxabicyclo[2.1.1]hexan-4-yl)-6-methylpyrimidin-4-yl)amino)-5- (methoxymethyl)-[2,3′-bipyridin]-6′-yl)acetamide 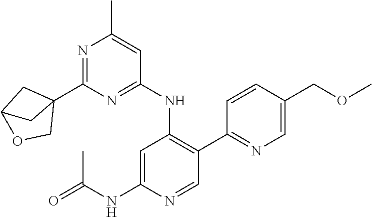 [SMILES: COCc1ccc(-c2cnc(NC(C)=O)cc2Nc2cc(C)nc(C34COC(C3)C4)n2)nc1] LCMS m/z = 447 [M + H]+ 1H NMR (CDCl₃, 400 MHz) δ 12.4-12.6 (m, 1H), 9.49 (s, 1H), 8.67 (d, 1H, J = 1.5 Hz), 8.55 (s, 1H), 8.17 (br s, 1H), 7.8-7.9 (m, 1H), 7.7-7.8 (m, 1H), 6.56 (s, 1H), 4.67 (s, 1H), 4.55 (s, 2H), 4.17 (s, 2H), 3.49 (s, 3H), 2.4-2.5 (m, 5H), 2.24 (s, 3H), 2.0-2.1 (m, 2H). |
| 566 | N-(4′-((2-(2-oxabicyclo[2.1.1]hexan-4-yl)-6-ethylpyrimidin-4-yl)amino)-5- (methoxymethyl)-[2,3′-bipyridin]-6′-yl)acetamide 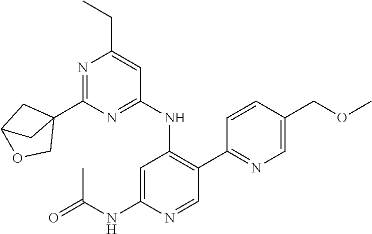 [SMILES: CCc1cc(Nc2cc(NC(C)=O)ncc2-c2ccc(COC)cn2)nc(C23COC(C2)C3)n1] LCMS m/z = 461 [M + H]⁺ 1H NMR (CDCl₃, 400 MHz) δ 12.52 (br s, 1H), 9.3-9.9 (m, 1H), 8.69 (s, 1H), 8.54 (br s, 1H), 7.8-7.9 (m, 1H), 7.7-7.8 (m, 1H), 6.5-6.6 (m, 1H), 4.6-4.7 (m, 1H), 4.56 (s, 2H), 4.1-4.2 (m, 2H), 3.49 (s, 3H), 2.7-2.8 (m, 2H), 2.4-2.5 (m, 2H), 2.25 (s, 3H), 2.03 (dd, 2H, J = 1.5, 4.5 Hz), 1.31 (t, 3H, J = 7.5 Hz). |
| 567 | N-(5-(methoxymethyl)-4′-((2-(tetrahydrofuran-3-yl)pyrimidin-4-yl)amino)-[2,3′-bipyridin]- 6′-yl)acetamide 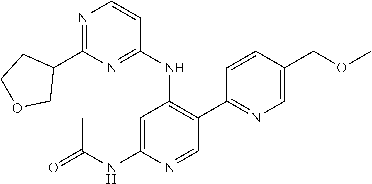 [SMILES: COCc1ccc(-c2cnc(NC(C)=O)cc2Nc2ccnc(C3CCOC3)n2)nc1] LCMS m/z = 421 [M + H]⁺ 1H NMR (MeOH-d4, 400 MHz) δ 9.3-9.4 (m, 1H), 8.6-8.7 (m, 2H), 8.33 (d, 1H, J = 6.0 Hz), 7.9-8.0 (m, 2H), 6.87 (d, 1H, J = 6.0 Hz), 4.56 (s, 2H), 4.2- 4.3 (m, 1H), 4.0-4.1 (m, 2H), 3.9-4.0 (m, 1H), 3.67 (quin, 1H, J = 7.8 Hz), 3.45 (s, 3H), 2.4- 2.6 (m, 1H), 2.37 (dddd, 1H, J = 5.5, 7.3, 8.5, 12.4 Hz), 2.21 (s, 3H). |
| 568 | N-(5-(methoxymethyl)-4′-((6-methyl-2-(tetrahydrofuran-3-yl)pyrimidin-4-yl)amino)-[2,3′- bipyridin]-6′-yl)acetamide 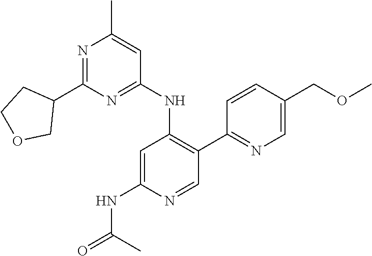 [SMILES: COCc1ccc(-c2cnc(NC(C)=O)cc2Nc2cc(C)nc(C3CCOC3)n2)nc1] LCMS m/z = 435 [M + H]⁺ 1H NMR (MeOH-d4, 400 MHz) δ 9.3-9.4 (m, 1H), 8.7 (m, 1H), 8.63 (s, 1H), 7.9-8.0 (m, 2H), 6.71 (s, 1H), 4.55 (s, 2H), 4.2-4.3 (m, 1H), 4.0-4.1 (m, 2H), 3.92 (q, 1H, J = 7.3 Hz), 3.61 (quin, 1H, J = 7.9 Hz), 3.46 (s, 3H), 2.5-2.6 (m, 1H), 2.40 (s, 3H), 2.3-2.4 (m, 1H), 2.2 (s, 3H). |
| 569 | N-(4′-((6-ethyl-2-(tetrahydrofuran-3-yl)pyrimidin-4-yl)amino)-5-(methoxymethyl)-[2,3′- bipyridin]-6′-yl)acetamide 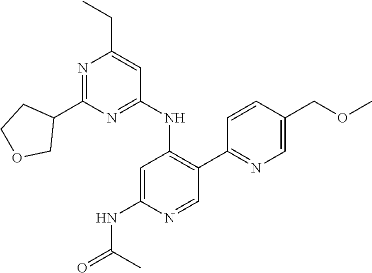 [SMILES: CCc1cc(Nc2cc(NC(C)=O)ncc2-c2ccc(COC)cn2)nc(C2CCOC2)n1] LCMS m/z = 449 [M + H]+ 1H NMR (MeOH-d4, 400 MHz) δ 9.30 (s, 1H), 8.70 (s, 1H), 8.63 (s, 1H), 7.9-8.0 (m, 2H), 6.73 (s, 1H), 4.56 (s, 2H), 4.2-4.3 (m, 1H), 4.0-4.1 (m, 2H), 3.93 (q, 1H, J = 7.3 Hz), 3.63 (quin, 1H, J = 7.8 Hz), 3.46 (s, 3H), 2.69 (q, 2H, J = 7.8 Hz), 2.52 (qd, 1H, J = 7.5, 12.3 Hz), 2.3-2.4 (m, 1H), 2.21 (s, 3H), 1.29 (t, 3H, J = 7.6 Hz). |
| 570 | N-(5-(methoxymethyl)-4′-((6-(methoxymethyl)-2-(tetrahydrofuran-3-yl)pyrimidin-4- yl)amino)-[2,3′-bipyridin]-6′-yl)acetamide 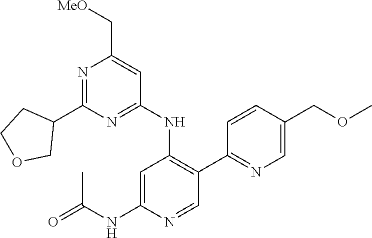 [SMILES: COCc1ccc(-c2cnc(NC(C)=O)cc2Nc2cc(COC)nc(C3CCOC3)n2)nc1] LCMS m/z = 465 [M + H]+ 1H NMR (MeOH-d4, 400 MHz) δ 9.41 (s, 1H), 8.7-8.8 (m, 1H), 8.6-8.7 (m, 1H), 7.90 (s, 2H), 6.87 (s, 1H), 4.57 (s, 2H), 4.44 (d, 2H, J = 0.8 Hz), 4.2-4.3 (m, 1H), 4.0-4.1 (m, 2H), 3.92 (q, 1H, J = 7.3 Hz), 3.6-3.7 (m, 1H), 3.5 (s, 3H), 3.46 (s, 3H), 2.51 (qd, 1H, J = 7.5, 12.3 Hz), 2.3-2.4 (m, 1H), 2.2 (s, 3H). |
| 571 | N-(5-(methoxymethyl)-4′-((6-methyl-2-(tetrahydrO2H-pyran-3-yl)pyrimidin-4-yl)amino)- [2,3′-bipyridin]-6′-yl)acetamide 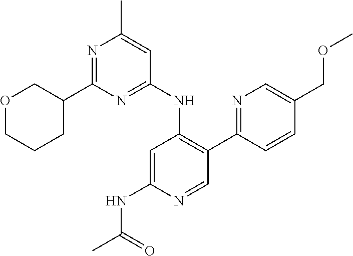 [SMILES: COCc1ccc(-c2cnc(NC(C)=O)cc2Nc2cc(C)nc(C3CCCOC3)n2)nc1] LCMS m/z = 449 [M + H]+; 1H NMR (MeOH-d4, 400 MHz) δ 9.3-9.5 (m, 1H), 8.6-8.7 (m, 1H), 8.6 (s, 1H), 7.8-7.9 (m, 2H), 6.64 (s, 1H), 4.54 (s, 2H), 4.0-4.1 (m, 1H), 3.94 (d, 2H, J = 10.8 Hz), 3.6-3.7 (m, 1H), 3.45 (s, 3H), 2.9-3.1 (m, 1H), 2.38 (s, 3H), 2.21 (s, 3H), 2.0-2.2 (m, 2H), 1.7-1.9 (m, 2H). |
| 572 | N-(4′-((2-(2-oxabicyclo[2.1.1]hexan-4-yl)-6-methylpyrimidin-4-yl)amino)-5- (difluoromethyl)-[2,3′-bipyridin]-6′-yl)acetamide 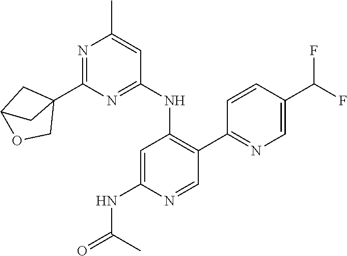 [SMILES: CC(=O)Nc1cc(Nc2cc(C)nc(C34COC(C3)C4)n2)c(-c2ccc(C(F)F)cn2)cn1] LCMS m/z = 453 [M + H]⁺ 1H NMR (MeOH-d4, 400 MHz) δ 9.44 (s, 1H), 8.93 (d, 1H, J = 0.8 Hz), 8.71 (s, 1H), 8.1-8.2 (m, 2H), 6.98 (t, 1H, J = 55.5 Hz), 6.79 (d, 1H, J = 0.8 Hz), 4.62 (t, 1H, J = 1.0 Hz), 4.10 (s, 2H), 2.4-2.5 (m, 5H), 2.21 (s, 3H), 1.96-1.98 (m, 2H). |
| 573 | N-(4-((2-(1,1-difluoroethyl)pyrimidin-4-yl)amino)-5-(6,7-dihydrO4H-pyrano[4,3-d]thiazol- 2-yl)pyridin-2-yl)acetamide 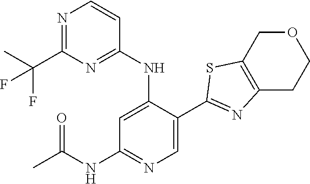 [SMILES: CC(=O)Nc1cc(Nc2ccnc(C(C)(F)F)n2)c(-c2nc3c(s2)COCC3)cn1] LCMS m/z = 433 [M + H]⁺ |
| 574 | N-(4′-((2-(2-oxabicyclo[2.1.1]hexan-4-yl)-6-methylpyrimidin-4-yl)amino)-5- morpholinO[2,3′-bipyridin]-6′-yl)acetamide 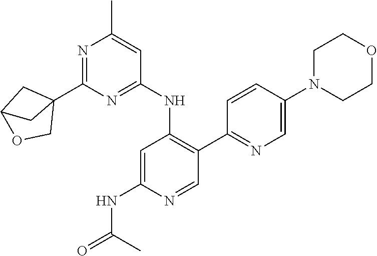 [SMILES: CC(=O)Nc1cc(Nc2cc(C)nc(C34COC(C3)C4)n2)c(-c2ccc(N3CCOCC3)cn2)cn1] LCMS m/z = 488 [M + H]⁺ |
| 575 | N-(4′-((2-(2-oxabicyclo[2.1.1]hexan-4-yl)-6-methylpyrimidin-4-yl)amino)-5-(1- methoxyethyl)-[2,3′-bipyridin]-6′-yl)acetamide 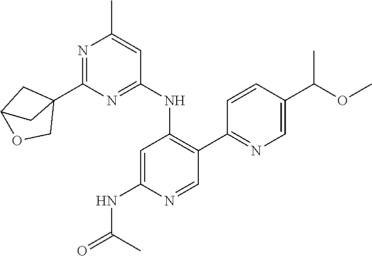 [SMILES: COC(C)c1ccc(-c2cnc(NC(C)=O)cc2Nc2cc(C)nc(C34COC(C3)C4)n2)nc1] LCMS m/z = 461 [M + H]⁺; 1H NMR (MeOH-d4, 400 MHz) δ 9.4-9.5 (m, 1H), 8.70 (d, 1H, J = 1.8 Hz), 8.65 (s, 1H), 7.9-8.0 (m, 2H), 6.76 (d, 1H, J = 0.8 Hz), 4.62 (t, 1H, J = 1.0 Hz), 4.50 (q, 1H, J = 6.5 Hz), 4.10 (s, 2H), 3.30 (s, 3H), 2.4-2.5 (m, 5H), 2.21 (s, 3H), 1.96-1.98 (m, 2H), 1.49 (d, 3H, J = 6.5 Hz). |
| 576 | N-(4′-((2-(2-oxabicyclo[2.1.1]hexan-4-yl)-6-methylpyrimidin-4-yl)amino)-4- (difluoromethyl)-[2,3′-bipyridin]-6′-yl)acetamide 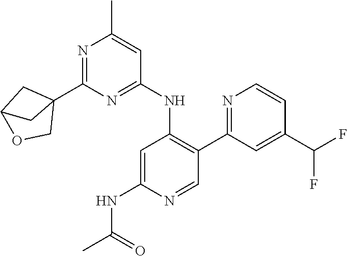 [SMILES: CC(=O)Nc1cc(Nc2cc(C)nc(C34COC(C3)C4)n2)c(-c2cc(C(F)F)ccn2)cn1] LCMS m/z = 453 [M + H]⁺. 1H NMR (MeOH-d4, 400 MHz) δ 9.4-9.5 (m, 1H), 8.89 (d, 1H, J = 5.3 Hz), 8.70 (s, 1H), 8.09 (s, 1H), 7.56 (d, 1H, J = 5.0 Hz), 6.8-7.1 (m, 1H), 6.8 (m, 1H), 4.62 (t, 1H, J = 1.0 Hz), 4.10 (s, 2H), 2.4-2.5 (m, 5H), 2.21 (s, 3H), 1.96-1.98 (m, 2H). |
| 577 | N-(4′-((2-(2-oxabicyclo[2.1.1]hexan-4-yl)-6-methylpyrimidin-4-yl)amino)-5-(1-fluoroethyl)- [2,3′-bipyridin]-6′-yl)acetamide 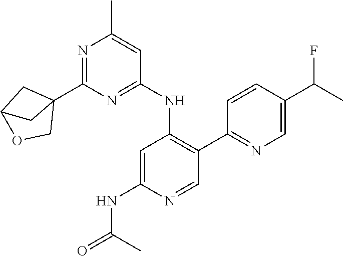 [SMILES: CC(=O)Nc1cc(Nc2cc(C)nc(C34COC(C3)C4)n2)c(-c2ccc(C(C)F)cn2)cn1] LCMS m/z = 449 [M + H]⁺ 1H NMR (MeOH-d4, 400 MHz) δ 9.42 (s, 1H), 8.76 (d, 1H, J = 1.5 Hz), 8.66 (s, 1H), 7.9-8.0 (m, 2H), 6.75 (s, 1H), 5.7-5.9 (m, 1H), 4.62 (t, 1H, J = 1.0 Hz), 4.10 (s, 2H), 2.4-2.5 (m, 5H), 2.21 (s, 3H), 1.96-1.98 (m, 2H), 1.7-1.8 (m, 3H). |
| 578 | N-(4-((2-(1,1-difluoroethyl)-6-methylpyrimidin-4-yl)amino)-5-(2-methyloxazol-4- yl)pyridin-2-yl)acetamide 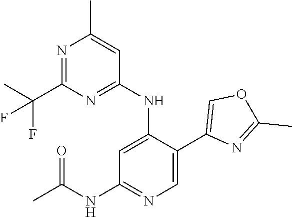 [SMILES: CC(=O)Nc1cc(Nc2cc(C)nc(C(C)(F)F)n2)c(-c2coc(C)n2)cn1] LCMS m/z = 389.2 [M + H]⁺; 1H NMR (600 MHz, DMSO-d₆) δ 10.69 (s, 1H), 10.53 (s, 1H), 9.07 (s, 1H), 8.57 (s, 1H), 8.51 (s, 1H), 6.98 (s, 1H), 2.57 (s, 3H), 2.43 (s, 3H), 2.11-2.03 (m, 6H) |
| 579 | N-(5-(1-cyclobutyl-1H-pyrazol-4-yl)-4-((2-(1,1-difluoroethyl)pyrimidin-4-yl)amino)pyridin- 2-yl)acetamide 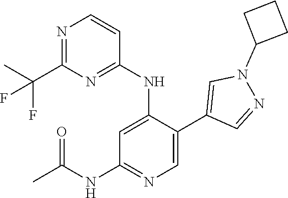 [SMILES: CC(=O)Nc1cc(Nc2ccnc(C(C)(F)F)n2)c(-c2cnn(C3CCC3)c2)cn1] LCMS m/z = 414.2 [M + H]⁺; 1H NMR (600 MHz, DMSO-d₆) δ 10.48 (s, 1H), 9.47 (s, 1H), 8.57 (s, 1H), 8.41 (d, J = 5.8 Hz, 1H), 8.35 (s, 1H), 8.12 (s, 1H), 7.71 (s, 1H), 2.46 (td, J = 9.6, 2.7 Hz, 2H), 2.37 (dq, J = 6.7, 3.8, 3.3 Hz, 2H), 2.08 (s, 3H), 1.95 (t, J = 19.0 Hz, 3H), 1.81-1.74 (m, 3H) |
| 580 | N-(4′-((2-(1,1-difluoroethyl)pyrimidin-4-yl)amino)-5-fluorO[2,3′-bipyridin]-6′-yl)acetamide 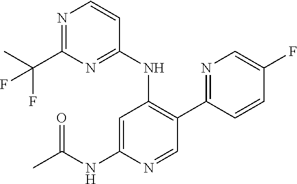 [SMILES: CC(=O)Nc1cc(Nc2ccnc(C(C)(F)F)n2)c(-c2ccc(F)cn2)cn1] LCMS m/z = 389.1 [M + H]⁺; 1H NMR (600 MHz, DMSO-d₆) δ 11.77 (s, 1H), 10.58 (s, 1H), 9.11 (s, 1H), 8.75 (d, J = 3.0 Hz, 1H), 8.69 (s, 1H), 8.54 (d, J = 5.8 Hz, 1H), 8.09-8.05 (m, 1H), 7.91 (td, J = 8.8, 3.0 Hz, 1H), 7.17 (d, J = 5.7 Hz, 1H), 2.13 (s, 3H), 2.06 (t, J = 19.2 Hz, 3H) |
| 581 | N-(4-((2-(1, 1-difluoroethyl)pyrimidin-4-yl)amino)-5-(2-methyloxazol-4-yl)pyridin-2- yl)acetamide 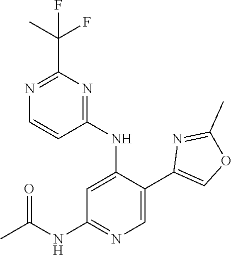 [SMILES: CC(=O)Nc1cc(Nc2ccnc(C(C)(F)F)n2)c(-c2coc(C)n2)cn1] LCMS m/z = 375.2 [M + H]⁺ |
| 582 | N-(4′-((2-(1,1-difluoroethyl)-6-ethylpyrimidin-4-yl)amino)-5-fluorO[2,3′-bipyridin]-6′- yl)acetamide 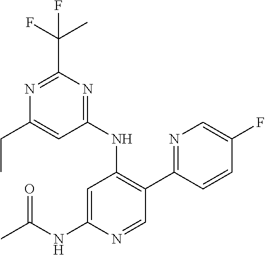 [SMILES: CCc1cc(Nc2cc(NC(C)=O)ncc2-c2ccc(F)cn2)nc(C(C)(F)F)n1] LCMS m/z = 417.2 [M + H]⁺, 1H NMR (500 MHz, DMSO-d₆) δ 11.77 (s, 1H), 10.58 (s, 1H), 9.14 (s, 1H), 8.78 (d, J = 2.7 Hz, 1H), 8.69 (d, J = 1.5 Hz, 1H), 8.09 (dd, J = 9.2, 4.2 Hz, 1H), 7.93 (tt, J = 9.1, 2.3 Hz, 1H), 7.05 (s, 1H), 2.69 (q, J = 7.6 Hz, 2H), 2.13-2.02 (m, 6H), 1.23 (td, J = 7.5, 1.7 Hz, 3H) |
| 583 | N-(5-cyanO4′-((2-(1,1-difluoroethyl)-6-ethylpyrimidin-4-yl)amino)-[2,3′-bipyridin]-6′- yl)acetamide 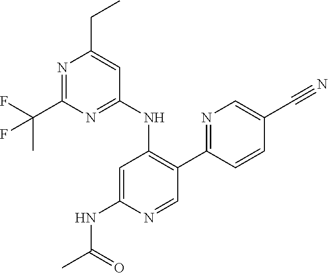 [SMILES: CCc1cc(Nc2cc(NC(C)=O)ncc2-c2ccc(C#N)cn2)nc(C(C)(F)F)n1] LCMS m/z = 424.1 [M + H]+ 1H NMR (600 MHz, DMSO) δ 11.84 (s, 1H), 10.65 (s, 1H), 9.18 (dd, J = 2.3, 0.9 Hz, 1H), 9.15 (s, 1H), 8.80 (s, 1H), 8.42 (dd, J = 8.5, 2.2 Hz, 1H), 8.21 (dd, J = 8.6, 0.9 Hz, 1H), 7.06 (s, 1H), 2.70 (q, J = 7.6 Hz, 2H), 2.13 (s, 3H), 2.05 (t, J = 19.2 Hz, 3H), 1.24 (t, J = 7.6 Hz, 3H) |
| 584 | N-(4-((2-(1,1-difluoroethyl)-6-ethylpyrimidin-4-yl)amino)-5-(2-methyloxazol-4-yl)pyridin- 2-yl)acetamide 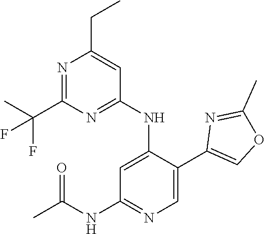 [SMILES: CCc1cc(Nc2cc(NC(C)=O)ncc2-c2coc(C)n2)nc(C(C)(F)F)n1] LCMS m/z = 403.1 [M + H]+ 1H NMR (500 MHz, DMSO) δ 10.63 (s, 1H), 10.51 (s, 1H), 9.06 (s, 1H), 8.56 (s, 1H), 8.51 (d, J = 3.3 Hz, 1H), 6.97 (s, 1H), 2.70 (q, J = 7.7 Hz, 2H), 2.57 (s, 3H), 2.12-2.02 (m, 6H), 1.23 (td, J = 7.6, 1.8 Hz, 3H) |
| 585 | N-(4-((2-(1,1-difluoroethyl)-6-(2-methoxyethoxy)pyrimidin-4-yl)amino)-5-(pyrimidin-4- yl)pyridin-2-yl)acetamide 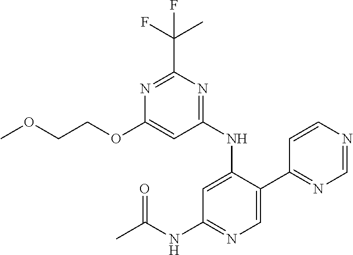 [SMILES: COCCOc1cc(Nc2cc(NC(C)=O)ncc2-c2ccncn2)nc(C(C)(F)F)n1] LCMS m/z = 446.1 [M + H]+ 1H NMR (400 MHz, DMSO) δ 12.25 (s, 1H), 10.65 (s, 1H), 9.33 (d, J = 1.3 Hz, 1H), 9.11 (s, 1H), 8.88-8.86 (m, 2H), 8.14 (dd, J = 5.6, 1.4 Hz, 1H), 6.65 (s, 1H), 4.48-4.46 (m, 2H), 3.68-3.65 (m, 2H), 3.30 (s, 3H), 2.13 (s, 3H), 2.06 (t, J = 19.2 Hz, 3H) |
| 586 | N-(5-fluorO4′-((6-methoxypyrazin-2-yl)amino)-[2,3′-bipyridin]-6′-yl)acetamide 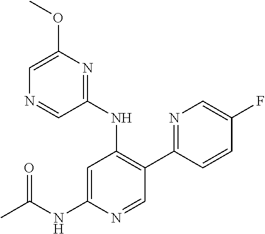 [SMILES: COc1cncc(Nc2cc(NC(C)=O)ncc2-c2ccc(F)cn2)n1] LCMS m/z = 355.0 [M + H]+ 1H NMR (600 MHz, DMSO) δ 12.14 (s, 1H), 10.49 (s, 1H), 9.25 (s, 1H), 8.80 (d, J = 3.0 Hz, 1H), 8.71 (s, 1H), 8.17 (dd, J = 9.1, 4.3 Hz, 1H), 8.06 (s, 1H), 7.94 (td, J = 8.8, 3.1 Hz, 1H), 7.78 (s, 1H), 4.03 (s, 3H), 2.11 (s, 3H). |
| 587 | N-(4-((6-cyclopropoxy-2-(1,1-difluoroethyl)pyrimidin-4-yl)amino)-5-(2-(2-methoxyethyl)- 2H-1,2,3-triazol-4-yl)pyridin-2-yl)acetamide 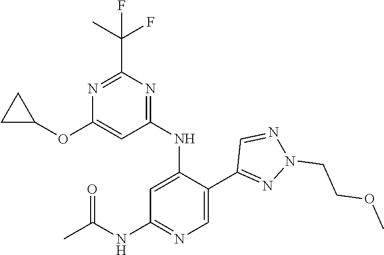 LCMS m/z = 475.3 [M + H]+ 1H NMR (600 MHz, DMSO) δ 10.57 (s, 1H), 10.25 (s, 1H), 8.96 (s, 1H), 8.70 (s, 1H), 8.32 (s, 1H), 6.66 (s, 1H), 4.69 (t, J = 5.2 Hz, 2H), 4.24 (tt, J = 6.3, 3.0 Hz, 1H), 3.87 (t, J = 5.3 Hz, 2H), 3.24 (s, 3H), 2.11 (s, 3H), 2.03 (t, J = 19.1 Hz, 3H), 0.85-0.81 (m, 2H), 0.78-0.75 (m, 2H) |
| 588 | N-(4′-((6-(difluoromethoxy)pyrazin-2-yl)amino)-5-(methoxymethyl)-[2,3′-bipyridin]-6′- yl)acetamide 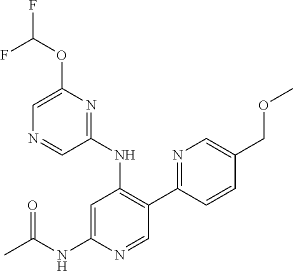 [SMILES: COCc1ccc(-c2cnc(NC(C)=O)cc2Nc2cncc(OC(F)F)n2)nc1] LCMS m/z = 417.3 [M + H]+ 1H NMR (600 MHz, DMSO) δ 13.29 (s, 1H), 10.64 (s, 1H), 9.21 (s, 1H), 8.85 (s, 1H), 8.78 (dd, J = 2.2, 0.9 Hz, 1H), 8.41 (d, J = 0.6 Hz, 1H), 8.30-7.86 (m, 4H), 4.54 (s, 2H), 3.37 (s, 3H), 2.15 (s, 3H) |
| 589 | N-(4′-((2-(1,1-difluoroethyl)-5-methoxypyrimidin-4-yl)amino)-5-(methoxymethyl)-[2,3′- bipyridin]-6′-yl)acetamide 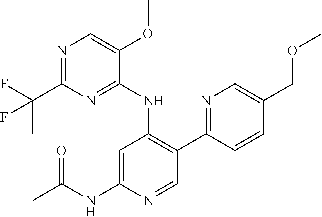 [SMILES: COCc1ccc(-c2cnc(NC(C)=O)cc2Nc2nc(C(C)(F)F)ncc2OC)nc1] LCMS m/z = 445.2 [M + H]⁺ |
| 590 | N-(4-((2-(1,1-difluoroethyl)-5-methoxypyrimidin-4-yl)amino)-5-(1-methyl-1H-pyrazol-3- yl)pyridin-2-yl)acetamide 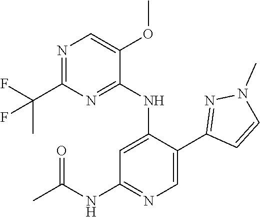 [SMILES: COc1cnc(C(C)(F)F)nc1Nc1cc(NC(C)=O)ncc1-c1ccn(C)n1] LCMS m/z = 404.2[M + H]⁺ |
| 591 | N-(4′-((2-(1,1-difluoroethyl)-6-methylpyrimidin-4-yl)amino)-[2,3′-bipyridin]-6′-yl)acetamide 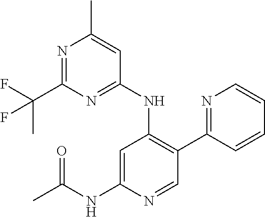 [SMILES: CC(=O)Nc1cc(Nc2cc(C)nc(C(C)(F)F)n2)c(-c2ccccn2)cn1] LCMS m/z = 385.3 [M + H]+ 1H NMR (600 MHz, DMSO) δ 12.75 (s, 1H), 10.56 (s, 1H), 9.24 (s, 1H), 8.79-8.77 (m, 2H), 8.09 (d, J = 8.2 Hz, 1H), 7.99 (td, J = 7.8, 1.9 Hz, 1H), 7.49-7.44 (m, 1H), 7.09 (d, J = 4.6 Hz, 1H), 2.43 (s, 3H), 2.15-2.04 (m, 6H) |
| 592 | N-(4-((2-(1,1-difluoroethyl)-6-methylpyrimidin-4-yl)amino)-5-(4-methyloxazol-2- yl)pyridin-2-yl)acetamide 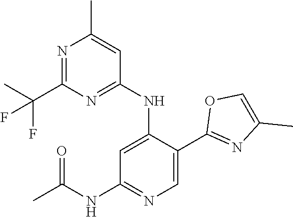 [SMILES: CC(=O)Nc1cc(Nc2cc(C)nc(C(C)(F)F)n2)c(-c2nc(C)co2)cn1] LCMS m/z = 389.1 [M + H]+ |
| 593 | N-(4′-((2-(1,1-difluoroethyl)-6-methylpyrimidin-4-yl)amino)-5-methyl-[2,3′-bipyridin]-6′- yl)acetamide 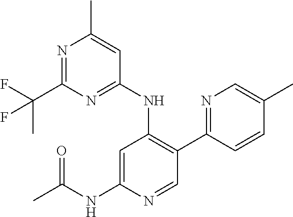 [SMILES: CC(=O)Nc1cc(Nc2cc(C)nc(C(C)(F)F)n2)c(-c2ccc(C)cn2)cn1] LCMS m/z = 399.3 [M + H]+ 1H NMR (600 MHz, DMSO) δ 12.77 (s, 1H), 10.57 (d, J = 14.8 Hz, 1H), 9.20 (s, 1H), 8.75 (s, 1H), 8.66-8.62 (m, 1H), 8.00 (d, J = 8.3 Hz, 1H), 7.84 (dd, J = 8.3, 2.5 Hz, 1H), 7.08 (d, J = 1.1 Hz, 1H), 2.43 (s, 3H), 2.39 (s, 3H), 2.14-2.05 (m, 6H) |
| 594 | N-(4′-((2-(1,1-difluoroethyl)-6-methylpyrimidin-4-yl)amino)-5-fluorO[2,3′-bipyridin]-6′- yl)acetamide 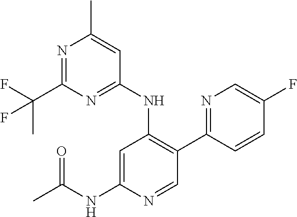 [SMILES: CC(=O)Nc1cc(Nc2cc(C)nc(C(C)(F)F)n2)c(-c2ccc(F)cn2)cn1] LCMS m/z = 403.1 [M + H]+ 1H NMR (500 MHz, DMSO) δ 11.87 (s, 1H), 10.69 (d, J = 13.0 Hz, 1H), 9.12 (s, 1H), 8.77 (d, J = 3.0 Hz, 1H), 8.69 (s, 1H), 8.09 (dd, J = 9.0, 4.2 Hz, 1H), 7.94 (td, J = 8.8, 2.9 Hz, 1H), 7.07 (s, 1H), 2.42 (s, 3H), 2.13 (s, 3H), 2.06 (t, J = 19.3 Hz, 3H) |
| 595 | N-(4-((2-(1,1-difluoroethyl)-6-methylpyrimidin-4-yl)amino)-5-(5-methylpyrimidin-2- yl)pyridin-2-yl)acetamide 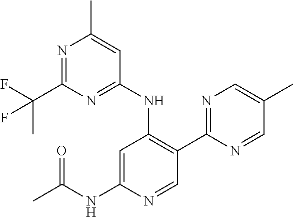 [SMILES: CC(=O)Nc1cc(Nc2cc(C)nc(C(C)(F)F)n2)c(-c2ncc(C)cn2)cn1] LCMS m/z = 400.3 [M + H]+ 1H NMR (600 MHz, DMSO) δ 12.92 (s, 1H), 10.67 (s, 1H), 9.35 (s, 1H), 9.31 (s, 1H), 8.87 (d, J = 1.0 Hz, 1H), 7.19 (s, 1H), 2.46 (s, 3H), 2.36 (s, 3H), 2.19-2.07 (m, 6H) |
| 596 | N-(4-((2-(1,1-difluoroethyl)-6-methylpyrimidin-4-yl)amino)-5-(oxazol-4-yl)pyridin-2- yl)acetamide 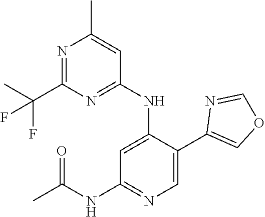 [SMILES: CC(=O)Nc1cc(Nc2cc(C)nc(C(C)(F)F)n2)c(-c2cocn2)cn1] LCMS m/z = 375.0 [M + H]+ 1H NMR (600 MHz, DMSO) δ 10.57 (s, 1H), 10.37 (s, 1H), 8.97 (s, 1H), 8.68 (d, J = 0.9 Hz, 1H), 8.64 (d, J = 1.0 Hz, 1H), 8.60 (s, 1H), 6.95 (d, J = 0.9 Hz, 1H), 2.43-2.41 (m, 3H), 2.11 (s, 3H), 2.04 (t, J = 19.2 Hz, 3H) |
| 597 | N-(4-((2-(1,1-difluoroethyl)-6-methylpyrimidin-4-yl)amino)-5-(3-methyl-1H-pyrazol-1- yl)pyridin-2-yl)acetamide 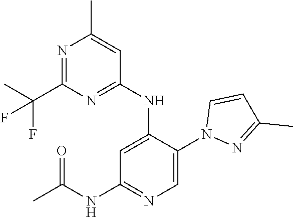 [SMILES: CC(=O)Nc1cc(Nc2cc(C)nc(C(C)(F)F)n2)c(-n2ccc(C)n2)cn1] 388.3(M + H) 1H NMR (600 MHz, DMSO) δ 10.57 (s, 1H), 10.22 (s, 1H), 9.04 (s, 1H), 8.38 (s, 1H), 8.13 (d, J = 2.4 Hz, 1H), 6.92 (s, 1H), 6.34 (d, J = 2.4 Hz, 1H), 2.40 (s, 3H), 2.32 (s, 3H), 2.12-1.99 (m, 6H) |
| 598 | N-(4-((2-(1,1-difluoroethyl)-6-methylpyrimidin-4-yl)amino)-5-(5-fluoropyrazin-2- yl)pyridin-2-yl)acetamide 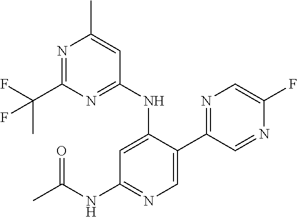 [SMILES: CC(=O)Nc1cc(Nc2cc(C)nc(C(C)(F)F)n2)c(-c2cnc(F)cn2)cn1] LCMS m/z = 404.3 [M + H]+ 1H NMR (600 MHz, DMSO) δ 10.63 (s, 1H), 10.39 (s, 1H), 8.93 (s, 1H), 8.76 (dd, J = 8.3, 1.3 Hz, 1H), 8.74 (d, J = 1.5 Hz, 1H), 8.60 (s, 1H), 6.91 (s, 1H), 2.39 (s, 3H), 2.13 (s, 3H), 1.98 (t, J = 19.1 Hz, 3H) |
| 599 | N-(4′-((2-(1,1-difluoroethyl)-6-methylpyrimidin-4-yl)amino)-5-methoxy-[2,3′-bipyridin]-6′- yl)acetamide 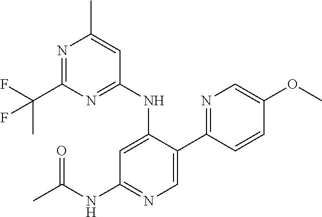 [SMILES: COc1ccc(-c2cnc(NC(C)=O)cc2Nc2cc(C)nc(C(C)(F)F)n2)nc1] LCMS m/z = 415.3 [M + H]+ 1H NMR (600 MHz, DMSO) δ 12.53 (s, 1H), 10.49 (s, 1H), 9.22 (s, 1H), 8.71 (s, 1H), 8.52 (d, J = 3.0 Hz, 1H), 8.04 (d, J = 9.0 Hz, 1H), 7.60 (dd, J = 8.9, 3.1 Hz, 1H), 7.08 (s, 1H), 3.92 (s, 3H), 2.43 (s, 3H), 2.13-2.06 (m, 6H) |
| 600 | N-(4-((2-(1,1-difluoroethyl)-6-methylpyrimidin-4-yl)amino)-5-(2-methyloxazol-5- yl)pyridin-2-yl)acetamide 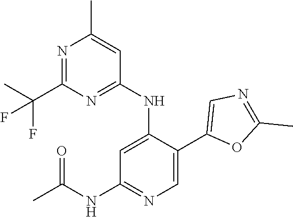 [SMILES: CC(=O)Nc1cc(Nc2cc(C)nc(C(C)(F)F)n2)c(-c2cnc(C)o2)cn1] LCMS m/z = 389.1 [M + H]+, 1H NMR (500 MHz, DMSO) δ 10.63 (s, 1H), 9.53 (s, 1H), 8.67 (d, J = 3.5 Hz, 1H), 8.44 (d, J = 3.8 Hz, 1H), 7.26 (d, J = 3.8 Hz, 1H), 6.85 (d, J = 3.7 Hz, 1H), 2.41-2.37 (m, 6H), 2.10 (d, J = 3.7 Hz, 3H), 1.98-1.88 (m, 3H) |
| 601 | N-(5-(2-cyanO1-methyl-1H-imidazol-4-yl)-4-((2-(1,1-difluoroethyl)-6-methylpyrimidin-4- yl)amino)pyridin-2-yl)acetamide 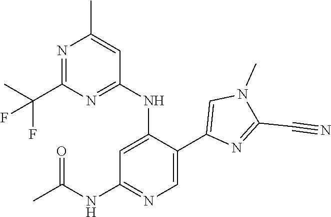 [SMILES: CC(=O)Nc1cc(Nc2cc(C)nc(C(C)(F)F)n2)c(-c2cn(C)c(C#N)n2)cn1] LCMS m/z = 413 [M + H]+ |
| 602 | N-(5-(1-cyclopropyl-1H-pyrazol-3-yl)-4-((2-(1,1-difluoroethyl)-6-methylpyrimidin-4- yl)amino)pyridin-2-yl)acetamide 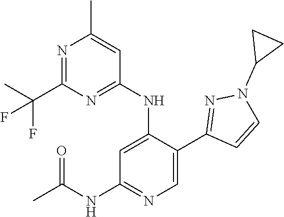 [SMILES: CC(=O)Nc1cc(Nc2cc(C)nc(C(C)(F)F)n2)c(-c2ccn(C3CC3)n2)cn1] LCMS m/z = 414.2 [M + H]+ 1H NMR (500 MHz, DMSO) δ 11.37 (s, 1H), 10.47 (s, 1H), 9.21 (s, 1H), 8.66 (s, 1H), 7.99 (s, 1H), 6.92 (s, 2H), 3.96-3.89 (m, 1H), 2.46 (s, 3H), 2.16- 2.05 (m, 6H), 1.24-1.17 (m, 3H), 1.07 (d, J = 7.3 Hz, 1H) |
| 603 | N-(4-((2-(1,1-difluoroethyl)-6-methylpyrimidin-4-yl)amino)-5-(1-isopropyl-1H-pyrazol-3- yl)pyridin-2-yl)acetamide 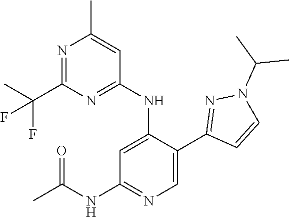 [SMILES: CC(=O)Nc1cc(Nc2cc(C)nc(C(C)(F)F)n2)c(-c2ccn(C(C)C)n2)cn1] LCMS m/z = 416.1 [M + H]+ 1H NMR (600 MHz, DMSO) δ 11.55 (s, 1H), 10.45 (s, 1H), 9.22 (s, 1H), 8.68 (s, 1H), 7.98 (d, J = 2.4 Hz, 1H), 6.95 (s, 1H), 6.93 (d, J = 2.4 Hz, 1H), 4.70 (p, J = 6.7 Hz, 1H), 2.46 (s, 3H), 2.17-2.06 (m, 6H), 1.54 (s, 3H), 1.52 (s, 3H) |
| 604 | N-(5-(1-cyclobutyl-1H-pyrazol-4-yl)-4-((2-(1,1-difluoroethyl)-6-methylpyrimidin-4- yl)amino)pyridin-2-yl)acetamide 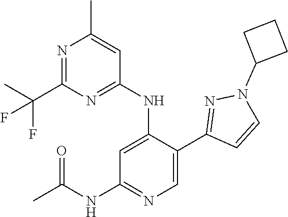 [SMILES: CC(=O)Nc1cc(Nc2cc(C)nc(C(C)(F)F)n2)c(-c2ccn(C3CCC3)n2)cn1] LCMS m/z = 428.1 [M + H] 1H NMR (600 MHz, DMSO) δ 10.47 (s, 1H), 9.31 (s, 1H), 8.55 (s, 1H), 8.33 (s, 1H), 8.10 (s, 1H), 7.70 (s, 1H), 6.77 (s, 1H), 2.45 (qd, J = 9.4, 2.5 Hz, 2H), 2.37 (tt, J = 10.9, 4.1 Hz, 3H), 2.34 (s, 3H), 2.08 (s, 3H), 1.94 (t, J = 19.0 Hz, 3H), 1.78 (ddt, J = 12.8, 10.4, 5.5 Hz, 2H) |
| 605 | N-(4-((2-(1,1-difluoroethyl)-6-methylpyrimidin-4-yl)amino)-5-(thiazol-2-yl)pyridin-2- yl)acetamide 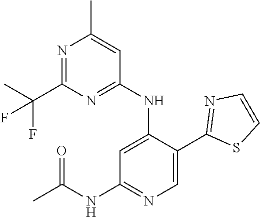 [SMILES: CC(=O)Nc1cc(Nc2cc(C)nc(C(C)(F)F)n2)c(-c2nccs2)cn1] LCMS m/z = 391 [M + H]+ |
| 606 | N-(4-((2-(1,1-difluoroethyl)-6-methylpyrimidin-4-yl)amino)-5-(2-methylthiazol-4- yl)pyridin-2-yl)acetamide 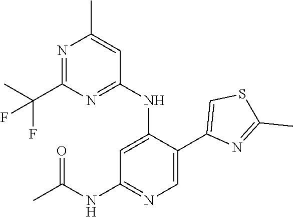 [SMILES: CC(=O)Nc1cc(Nc2cc(C)nc(C(C)(F)F)n2)c(-c2csc(C)n2)cn1] LCMS m/z = 405.2 [M + H]+ 1H NMR (600 MHz, DMSO) δ 11.44 (s, 1H), 10.55 (s, 1H), 9.15 (s, 1H), 8.69 (s, 1H), 8.06 (s, 1H), 6.99 (d, J = 0.9 Hz, 1H), 2.86 (s, 3H), 2.44 (s, 3H), 2.13-2.05 (m, 6H) |
| 607 | N-(4-((2-(1,1-difluoroethyl)-6-methylpyrimidin-4-yl)amino)-5-(1-methyl-1H-1,2,3-triazol-4- yl)pyridin-2-yl)acetamide 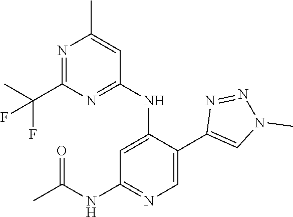 LCMS m/z = 389.1 [M + H]+ 1H NMR (500 MHz, DMSO) δ 10.93 (s, 1H), 10.51 (d, J = 2.6 Hz, 1H), 9.16 (s, 1H), 8.69 (d, J = 2.1 Hz, 1H), 8.61 (s, 1H), 7.02 (s, 1H), 4.15 (s, 3H), 2.44 (s, 3H), 2.13-2.03 (m, 6H) |
| 608 | N-(4-((2-(1,1-difluoroethyl)-6-methylpyrimidin-4-yl)amino)-5-(2-(methoxymethyl)-2H- 1,2,3-triazol-4-yl)pyridin-2-yl)acetamide 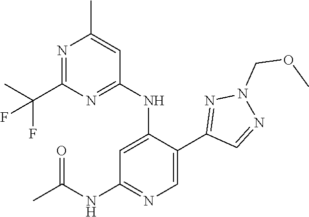 [SMILES: COCn1ncc(-c2cnc(NC(C)=O)cc2Nc2cc(C)nc(C(C)(F)F)n2)n1] LCMS m/z = 419.3 [M + H]+ 1H NMR (600 MHz, DMSO) δ 10.59 (s, 1H), 10.20 (s, 1H), 9.08 (s, 1H), 8.72 (s, 1H), 8.44 (s, 1H), 7.00 (s, 1H), 5.78 (s, 2H), 3.39-3.33 (m, 3H), 2.43 (s, 3H), 2.12 (s, 3H), 2.09-2.00 (m, 3H) |
| 609 | N-(4-((2-(1,1-difluoroethyl)-6-methylpyrimidin-4-yl)amino)-5-(5-methoxy-1-methyl-1H- 1,2,4-triazol-3-yl)pyridin-2-yl)acetamide 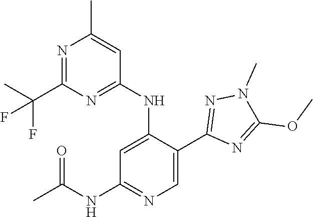 [SMILES: COc1nc(-c2cnc(NC(C)=O)cc2Nc2cc(C)nc(C(C)(F)F)n2)nn1C] LCMS m/z = 419.3 [M + H]+ 1H NMR (600 MHz, DMSO) δ 11.04 (s, 1H), 10.55 (s, 1H), 9.34 (s, 1H), 8.82 (s, 1H), 7.02 (s, 1H), 4.18 (s, 3H), 3.71 (s, 3H), 2.46 (s, 3H), 2.17-2.08 (m, 6H) |
| 610 | N-(4-((2-(1,1-difluoroethyl)-6-methylpyrimidin-4-yl)amino)-5-(2-isopropyl-2H-1,2,3- triazol-4-yl)pyridin-2-yl)acetamide 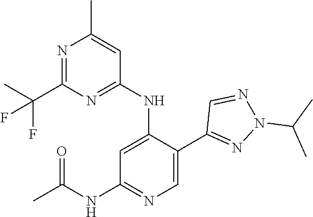 LCMS m/z = 417 [M + H]+ |
| 611 | N-(5-(5-(tert-butyl)-1,3,4-thiadiazol-2-yl)-4-((2-(1,1-difluoroethyl)-6-methylpyrimidin-4- yl)amino)pyridin-2-yl)acetamide 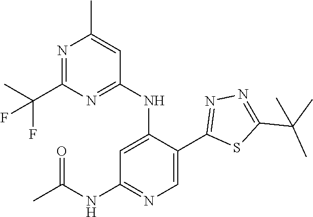 [SMILES: CC(=O)Nc1cc(Nc2cc(C)nc(C(C)(F)F)n2)c(-c2nnc(C(C)(C)C)s2)cn1] LCMS m/z = 448 [M + H]+ |
| 612 | N-(4-((2-(1,1-difluoroethyl)-6-methylpyrimidin-4-yl)amino)-5-(2-isopropylthiazol-4- yl)pyridin-2-yl)acetamide 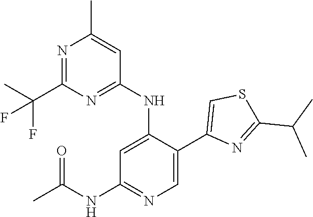 [SMILES: CC(=O)Nc1cc(Nc2cc(C)nc(C(C)(F)F)n2)c(-c2csc(C(C)C)n2)cn1] LCMS m/z = 433 [M + H]+ |
| 613 | N-(4-((2-(1,1-difluoroethyl)-6-methylpyrimidin-4-yl)amino)-5-(1-ethyl-1H-pyrazol-3- yl)pyridin-2-yl)acetamide 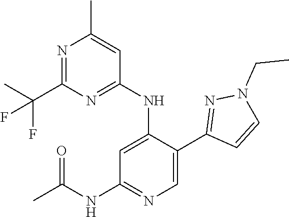 [SMILES: CCn1ccc(-c2cnc(NC(C)=O)cc2Nc2cc(C)nc(C(C)(F)F)n2)n1] LCMS m/z = 402 [M + H]+; 1H NMR (600 MHz, DMSO-d₆) δ 11.51 (s, 1H), 10.44 (s, 1H), 9.27 (s, 1H), 8.67 (s, 1H), 7.95 (d, J = 2.4 Hz, 1H), 7.00 (s, 1H), 4.33 (q, J = 7.3 Hz, 2H), 2.46 (s, 3H), 2.17-2.07 (m, 6H), 1.49 (t, J = 7.3 Hz, 3H) |
| 614 | N-(5-(1-(cyclopropylmethyl)-1H-pyrazol-3-yl)-4-((2-(1,1-difluoroethyl)-6-methylpyrimidin- 4-yl)amino)pyridin-2-yl)acetamide 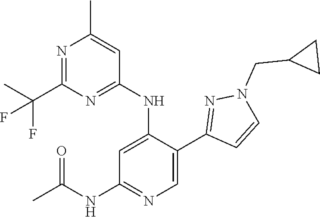 [SMILES: CC(=O)Nc1cc(Nc2cc(C)nc(C(C)(F)F)n2)c(-c2ccn(CC3CC3)n2)cn1] LCMS m/z = 428 [M + H]+ |
| 615 | N-(4-((2-(1,1-difluoroethyl)-6-methylpyrimidin-4-yl)amino)-5-(1-propyl-1H-pyrazol-3- yl)pyridin-2-yl)acetamide 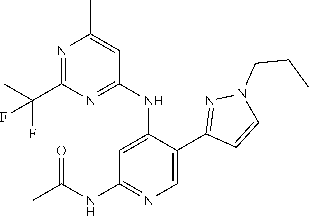 [SMILES: CCCn1ccc(-c2cnc(NC(C)=O)cc2Nc2cc(C)nc(C(C)(F)F)n2)n1] LCMS m/z = 416 [M + H]+ |
| 616 | N-(4-((2-(1,1-difluoroethyl)-6-methylpyrimidin-4-yl)amino)-5-(4-fluorO1-methyl-1H- pyrazol-3-yl)pyridin-2-yl)acetamide 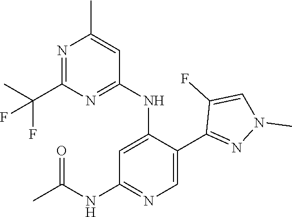 [SMILES: CC(=O)Nc1cc(Nc2cc(C)nc(C(C)(F)F)n2)c(-c2nn(C)cc2F)cn1] LCMS m/z = 406 [M + H]+ |
| 617 | N-(4-((2-(1,1-difluoroethyl)-6-methylpyrimidin-4-yl)amino)-5-(2-methoxythiazol-4- yl)pyridin-2-yl)acetamide 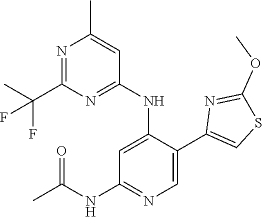 [SMILES: COc1nc(-c2cnc(NC(C)=O)cc2Nc2cc(C)nc(C(C)(F)F)n2)cs1] LCMS m/z = 421 [M + H]+ |
| 618 | N-(4-((2-(1,1-difluoroethyl)-6-methylpyrimidin-4-yl)amino)-5-(3-methyl-1,2,4-thiadiazol-5- yl)pyridin-2-yl)acetamide 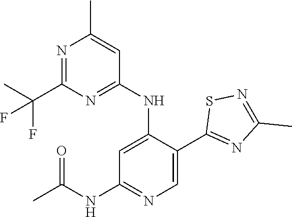 [SMILES: CC(=O)Nc1cc(Nc2cc(C)nc(C(C)(F)F)n2)c(-c2nc(C)ns2)cn1] LCMS m/z = 406 [M + H]+ |
| 619 | N-(4-((2-(1,1-difluoroethyl)-6-methylpyrimidin-4-yl)amino)-5-(1,5-dimethyl-1H-pyrazol-3- yl)pyridin-2-yl)acetamide 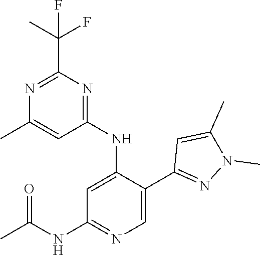 LCMS m/z = 402.2 [M + H]+ 1H NMR (DMSO-d6, 400 MHz): δ (ppm) 11.61 (s, 1H), 10.41 (s, 1H), 9.34 (s, 1H), 8.60 (s, 1H), 7.07 (s, 1H), 6.73 (s, 1H), 3.93 (s, 3H), 2.47 (s, 3H), 2.34 (s, 3H), 2.08-2.21 (m, 6H) |
| 620 | N-(4-((2-(1,1-difluoroethyl)-6-methylpyrimidin-4-yl)amino)-5-(pyrazin-2-yl)pyridin-2- yl)acetamide 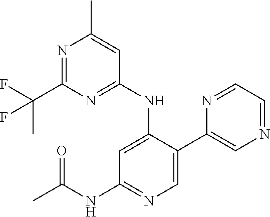 [SMILES: CC(=O)Nc1cc(Nc2cc(C)nc(C(C)(F)F)n2)c(-c2cnccn2)cn1] LCMS m/z = 386.1 [M + H]+ |
| 621 | N-(4-((2-(1,1-difluoroethyl)-6-methylpyrimidin-4-yl)amino)-5-(4-methoxy-6- methylpyrimidin-2-yl)pyridin-2-yl)acetamide 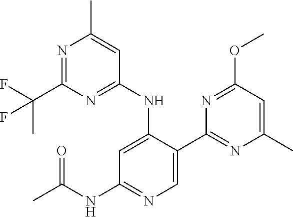 [SMILES: COc1cc(C)nc(-c2cnc(NC(C)=O)cc2Nc2cc(C)nc(C(C)(F)F)n2)n1] LCMS m/z = 430.1 [M + H]+ |
| 622 | N-(4′-((2-(1,1-difluoroethyl)-6-methylpyrimidin-4-yl)amino)-6-fluorO5-methoxy-[2,3′- bipyridin]-6′-yl)acetamide 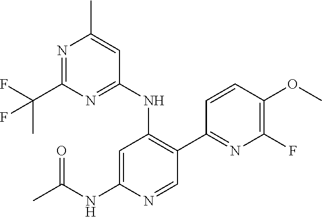 [SMILES: COc1ccc(-c2cnc(NC(C)=O)cc2Nc2cc(C)nc(C(C)(F)F)n2)nc1F] LCMS m/z = 433.1 [M + H]+ 1H NMR (600 MHz, DMSO) δ 10.55 (s, 1H), 10.24 (s, 1H), 8.88 (s, 1H), 8.49 (s, 1H), 7.76 (dd, J = 10.6, 8.3 Hz, 1H), 7.69 (d, J = 8.2 Hz, 1H), 6.85 (s, 1H), 3.92 (s, 3H), 2.39 (s, 3H), 2.11 (s, 3H), 1.99 (t, J = 19.1 Hz, 3H) |
| 623 | N-(4′-((2-(1,1-difluoroethyl)-6-methylpyrimidin-4-yl)amino)-6-fluorO[2,3′-bipyridin]-6′- yl)acetamide 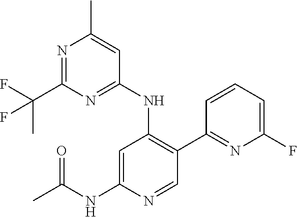 [SMILES: CC(=O)Nc1cc(Nc2cc(C)nc(C(C)(F)F)n2)c(-c2cccc(F)n2)cn1] LCMS m/z = 403.1 [M + H]+ |
| 624 | N-(4′-((2-(1,1-difluoroethyl)-6-methylpyrimidin-4-yl)amino)-4-fluorO[2,3′-bipyridin]-6′- yl)acetamide 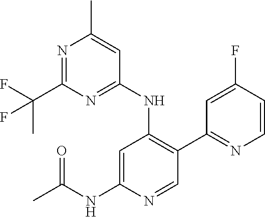 [SMILES: CC(=O)Nc1cc(Nc2cc(C)nc(C(C)(F)F)n2)c(-c2cc(F)ccn2)cn1] LCMS m/z = 403.2 [M + H]+ 1H NMR (600 MHz, DMSO) δ 12.60 (s, 1H), 10.58 (s, 1H), 9.25 (s, 1H), 8.82 (s, 1H), 8.07 (dd, J = 11.3, 2.4 Hz, 1H), 7.40 (ddd, J = 8.4, 5.8, 2.4 Hz, 1H), 7.10 (s, 1H), 2.43 (s, 3H), 2.14-2.05 (m, 6H) |
| 625 | N-(6′-bromO4-((2-(1,1-difluoroethyl)-6-methylpyrimidin-4-yl)amino)-[3,3′-bipyridin]-6- yl)acetamide 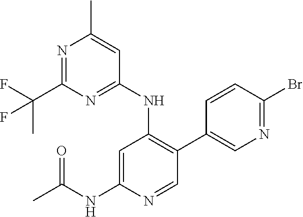 [SMILES: CC(=O)Nc1cc(Nc2cc(C)nc(C(C)(F)F)n2)c(-c2ccc(Br)nc2)cn1] LCMS m/z = 463.0 [M + H]+ 1H NMR (DMSO-d6, 400 MHz): δ (ppm) 10.58 (s, 1H), 9.47 (s, 1H), 8.65 (s, 1H), 8.44 (d, J = 2.5 Hz, 1H), 8.23 (s, 1H), 7.79 (dd, J = 8.0, 2.5 Hz, 1H), 7.68 (d, J = 7.5 Hz, 1H), 6.75 (s, 1H), 2.34 (s, 3H), 2.12 (s, 3H), 1.94 (t, J = 19.0 Hz, 3H) |
| 626 | N-(5-bromO4′-((2-(1,1-difluoroethyl)-6-methylpyrimidin-4-yl)amino)-[2,3′-bipyridin]-6′- yl)acetamide 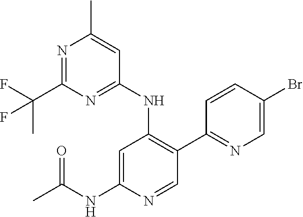 [SMILES: CC(=O)Nc1cc(Nc2cc(C)nc(C(C)(F)F)n2)c(-c2ccc(Br)cn2)cn1] LCMS m/z = 463.0 [M + H]+; 1H NMR (DMSO-d6, 400 MHz): δ (ppm) 11.94 (s, 1H), 10.54 (br s, 1H), 9.04-9.27 (m, 1H), 8.89 (s, 1H), 8.72 (s, 1H), 8.19 (br d, J = 8.5 Hz, 1H), 8.03 (br d, J = 9.0 Hz, 1H), 6.97-7.11 (m, 1H), 2.40 (s, 3H), 2.12 (s, 3H), 1.98-2.10 (m, 3H) |
| 627 | N-(4′-((2-(1,1-difluoroethyl)-6-methylpyrimidin-4-yl)amino)-4-(dimethylamino)-[2,3′- bipyridin]-6′-yl)acetamide 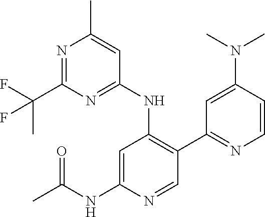 [SMILES: CC(=O)Nc1cc(Nc2cc(C)nc(C(C)(F)F)n2)c(-c2cc(N(C)C)ccn2)cn1] LCMS m/z = 428.1 [M + H]+ |
| 628 | N-(4′-((2-(1,1-difluoroethyl)-6-methylpyrimidin-4-yl)amino)-6-morpholinO[2,3′-bipyridin]- 6′-yl)acetamide 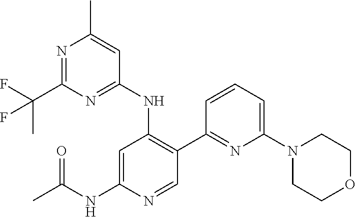 [SMILES: CC(=O)Nc1cc(Nc2cc(C)nc(C(C)(F)F)n2)c(-c2cccc(N3CCOCC3)n2)cn1] LCMS m/z = 470.2 [M + H]+ 1H NMR (DMSO-d6, 600 MHz): δ (ppm) 11.68 (s, 1H), 10.59 (s, 1H), 8.83 (s, 1H), 8.61 (s, 1H), 7.74 (dd, J = 8.4, 7.6 Hz, 1H), 7.23 (d, J = 7.2 Hz, 1H), 7.01 (s, 1H), 6.91 (d, J = 8.8 Hz, 1H), 3.77-3.81 (m, 4H), 3.56-3.59 (m, 4H), 2.43 (s, 3H), 2.13 (s, 3H), 2.04 (t, J = 19.1 Hz, 3H) |
| 629 | N-(4-((2-(1,1-difluoroethyl)-6-methylpyrimidin-4-yl)amino)-5-(2- (dimethylamino)pyrimidin-4-yl)pyridin-2-yl)acetamide 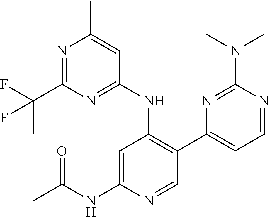 [SMILES: CC(=O)Nc1cc(Nc2cc(C)nc(C(C)(F)F)n2)c(-c2ccnc(N(C)C)n2)cn1] LCMS m/z = 429.2 [M + H]+ |
| 630 | N-(6-cyanO4′-((2-(1,1-difluoroethyl)-6-methylpyrimidin-4-yl)amino)-[2,3′-bipyridin]-6′- yl)acetamide 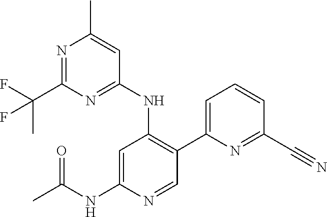 [SMILES: CC(=O)Nc1cc(Nc2cc(C)nc(C(C)(F)F)n2)c(-c2cccc(C#N)n2)cn1] LCMS m/z = 410.2 [M + H]⁺ |
| 631 | N-(4-cyanO4′-((2-(1,1-difluoroethyl)-6-methylpyrimidin-4-yl)amino)-[2,3′-bipyridin]-6′- yl)acetamide 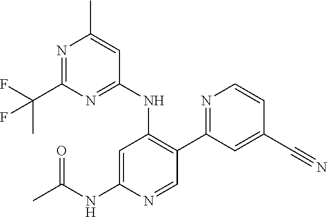 [SMILES: CC(=O)Nc1cc(Nc2cc(C)nc(C(C)(F)F)n2)c(-c2cc(C#N)ccn2)cn1] LCMS m/z = 410.2 [M + H]⁺ |
| 632 | N-(5-cyanO4′-((2-(1,1-difluoroethyl)-6-methylpyrimidin-4-yl)amino)-3-fluorO[2,3′- bipyridin]-6′-yl)acetamide 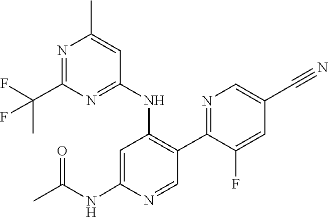 [SMILES: CC(=O)Nc1cc(Nc2cc(C)nc(C(C)(F)F)n2)c(-c2ncc(C#N)cc2F)cn1] LCMS m/z = 428.1 [M + H]+ |
| 633 | N-(6-cyanO4′-((2-(1,1-difluoroethyl)-6-methylpyrimidin-4-yl)amino)-5-fluorO[2,3′- bipyridin]-6′-yl)acetamide 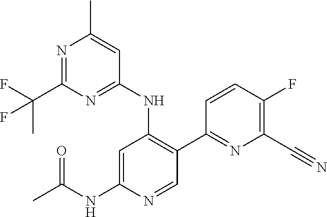 [SMILES: CC(=O)Nc1cc(Nc2cc(C)nc(C(C)(F)F)n2)c(-c2ccc(F)c(C#N)n2)cn1] LCMS m/z = 428.1 [M + H]+ |
| 634 | N-(4′-((2-(1,1-difluoroethyl)-6-methylpyrimidin-4-yl)amino)-5-(methoxymethyl)-[2,3′- bipyridin]-6′-yl)acetamide 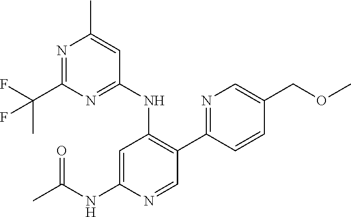 [SMILES: COCc1ccc(-c2cnc(NC(C)=O)cc2Nc2cc(C)nc(C(C)(F)F)n2)nc1] LCMS m/z = 429.3 [M + H]+ 1H NMR (600 MHz, DMSO) δ 12.79 (s, 1H), 10.55 (s, 1H), 9.25 (s, 1H), 8.79 (s, 1H), 8.75 (dd, J = 2.2, 0.9 Hz, 1H), 8.09 (dd, J = 8.5, 0.9 Hz, 1H), 7.92 (dd, J = 8.3, 2.3 Hz, 1H), 7.12 (d, J = 0.9 Hz, 1H), 4.54 (s, 2H), 3.37 (s, 3H), 2.43 (s, 3H), 2.15-2.05 (m, 6H) |
| 635 | N-(4′-((2-(1,1-difluoroethyl)-6-methylpyrimidin-4-yl)amino)-6-methoxy-[2,3′-bipyridin]-6′- yl)acetamide 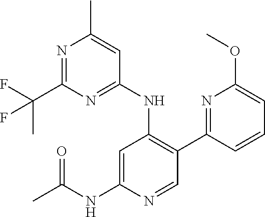 [SMILES: COc1cccc(-c2cnc(NC(C)=O)cc2Nc2cc(C)nc(C(C)(F)F)n2)n1] LCMS m/z = 415.2 [M + H]+ 1H NMR (MeOD-d4, 400 MHz): δ (ppm) 8.84 (s, 1H), 8.60 (s, 1H), 7.75-7.92 (m, 1H), 7.44 (d, J = 7.0 Hz, 1H), 7.08 (s, 1H), 6.76-6.91 (m,1H), 4.12 (s, 3H), 2.50 (s, 3H), 2.21 (s, 3H), 1.94-2.10 (m, 3H) |
| 636 | N-(4′-((2-(1,1-difluoroethyl)-6-methylpyrimidin-4-yl)amino)-6-(methoxymethyl)-[2,3′- bipyridin]-6′-yl)acetamide 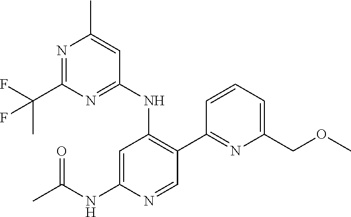 [SMILES: COCc1cccc(-c2cnc(NC(C)=O)cc2Nc2cc(C)nc(C(C)(F)F)n2)n1] LCMS m/z = 429.2 [M + H]+ 1H NMR (DMSO-d6, 400 MHz): δ (ppm) 13.20 (s, 1H), 10.55 (s, 1H), 9.31 (s, 1H), 8.85 (s, 1H), 7.95-8.13 (m, 2H), 7.44 (d, J = 7.5 Hz, 1H), 6.98 (s, 1H), 4.72 (s, 2H), 3.48 (s, 3H), 2.46 (s, 3H), 2.04-2.22 (m, 6H) |
| 637 | N-(4′-((2-(1,1-difluoroethyl)-6-methylpyrimidin-4-yl)amino)-5,6-dimethoxy-[2,3′-bipyridin]- 6′-yl)acetamide 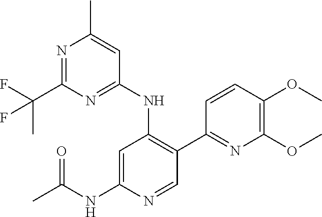 [SMILES: COc1ccc(-c2cnc(NC(C)=O)cc2Nc2cc(C)nc(C(C)(F)F)n2)nc1OC] LCMS m/z = 445.2 [M + H]+ 1H NMR (DMSO-d6, 400 MHz): δ (ppm) 10.87 (s, 1H), 10.54 (s, 1H), 8.84 (s, 1H), 8.56 (s, 1H), 7.46 (s, 2H), 7.03 (s, 1H), 4.02 (s, 3H), 3.85 (s, 3H), 2.41 (s, 3H), 2.13 (s, 3H), 2.02 (t, J = 19.0 Hz, 3H) |
| 638 | rac-(R)-N-(4′-((2-(1,1-difluoroethyl)-6-methylpyrimidin-4-yl)amino)-6-(1-hydroxyethyl)- [2,3′-bipyridin]-6′-yl)acetamide 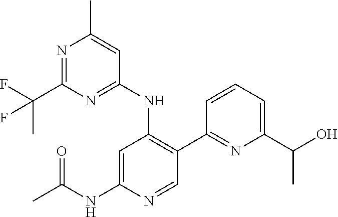 [SMILES: CC(=O)Nc1cc(Nc2cc(C)nc(C(C)(F)F)n2)c(-c2cccc(C(C)O)n2)cn1] LCMS m/z = 429.2 [M + H]+ 1H NMR (DMSO-d6, 600 MHz): δ (ppm) 13.33 (s, 1H), 10.54 (s, 1H), 9.26 (s, 1H), 8.84 (s, 1H), 7.90-8.13 (m, 2H), 7.46 (d, J = 7.2 Hz, 1H), 7.14 (s, 1H), 5.71 (br s, 1H), 4.86-5.16 (m, 1H), 2.43 (s, 3H), 2.05-2.18 (m, 6H), 1.49 (d, J = 6.5 Hz, 3H) |
| 639 | N-(4′-((2-(1,1-difluoroethyl)-6-methylpyrimidin-4-yl)amino)-6-(tetrahydrofuran-2-yl)-[2,3′- bipyridin]-6′-yl)acetamide 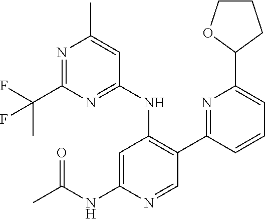 [SMILES: CC(=O)Nc1cc(Nc2cc(C)nc(C(C)(F)F)n2)c(-c2cccc(C3CCCO3)n2)cn1] LCMS m/z = 455.2 [M + H]+ 1H NMR (MeOD-d4, 400 MHz): δ (ppm) 9.23 (s, 1H), 8.73 (s, 1H), 7.93-8.01 (m, 1H), 7.83-7.91 (m, 1H), 7.47 (d, J = 7.5 Hz, 1H), 7.10 (s, 1H), 5.13- 5.23 (m, 1H), 4.19-4.28 (m, 1H), 4.04-4.14 (m, 1H), 2.54-2.64 (m, 1H), 2.52 (s, 3H), 2.24 (s, 3H), 2.02-2.19 (m, 6H) |
| 640 | N-(4′-((2-(1,1-difluoroethyl)-6-methylpyrimidin-4-yl)amino)-6-(4-methylmorpholin-2-yl)- [2,3′-bipyridin]-6′-yl)acetamide 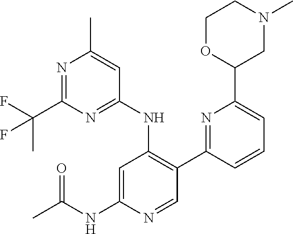 [SMILES: CC(=O)Nc1cc(Nc2cc(C)nc(C(C)(F)F)n2)c(-c2cccc(C3CN(C)CCO3)n2)cn1] LCMS m/z = 484.2 [M + H]+ 1H NMR (MeOD-d4, 400 MHz): δ (ppm) 9.19 (s, 1H), 8.59 (s, 1H), 7.87 (t, J = 8.0 Hz, 1H), 7.77-7.81 (m, 1H), 7.39 (d, J = 7.5 Hz, 1H), 6.90 (s, 1H), 4.71-4.74 (m, 1H), 4.06-4.17 (m, 1H), 3.87 (td, J = 11.8, 2.5 Hz, 1H), 3.11-3.16 (m, 1H), 2.85 (br d, J = 11.5 Hz, 1H), 2.41 (s, 3H), 2.35-2.37 (m, 1H), 2.33 (s, 3H), 2.17-2.26 (m, 1H), 2.11 (s, 3H), 1.93-2.08 (m, 3H) |
| 641 | (S)-N-(4′-((2-(1,1-difluoroethyl)-6-methylpyrimidin-4-yl)amino)-6-(4-methylmorpholin-2- yl)-[2,3′-bipyridin]-6′-yl)acetamide or (R)-N-(4′-((2-(1,1-difluoroethyl)-6-methylpyrimidin- 4-yl)amino)-6-(4-methylmorpholin-2-yl)-[2,3′-bipyridin]-6′-yl)acetamide 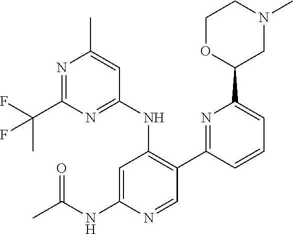 [SMILES: CC(=O)Nc1cc(Nc2cc(C)nc(C(C)(F)F)n2)c(-c2cccc([C@@H]3CN(C)CCO3)n2)cn1] or 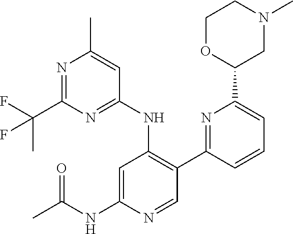 [SMILES: CC(=O)Nc1cc(Nc2cc(C)nc(C(C)(F)F)n2)c(-c2cccc([C@H]3CN(C)CCO3)n2)cn1] LCMS m/z = 484.2 [M + H]+ 1H NMR (MeOD-d₄, 400 MHz): δ (ppm) 9.32 (s, 1H), 8.70 (s, 1H), 7.87-8.01 (m, 2H), 7.49 (d, J = 7.5 Hz, 1H), 7.01 (s, 1H), 4.82 (dd, J = 10.5, 2.5 Hz, 1H), 4.16-4.24 (m, 1H), 3.96 (td, J = 11.5, 2.5 Hz, 1H), 3.18 (dt, J = 11.6, 1.9 Hz, 1H), 2.90 (dd, J = 11.8, 1.8 Hz, 1H), 2.51 (s, 3H), 2.39 (s, 3H), 2.32-2.37 (m, 1H), 2.06-2.27 (m, 7H) |
| 642 | (R)-N-(4′-((2-(1,1-difluoroethyl)-6-methylpyrimidin-4-yl)amino)-6-(4-methylmorpholin-2- yl)-[2,3′-bipyridin]-6′-yl)acetamide or (S)-N-(4′-((2-(1,1-difluoroethyl)-6-methylpyrimidin-4- yl)amino)-6-(4-methylmorpholin-2-yl)-[2,3′-bipyridin]-6′-yl)acetamide 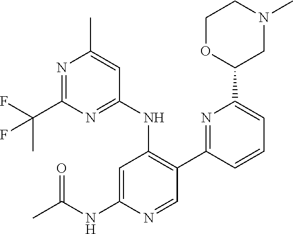 [SMILES: CC(=O)Nc1cc(Nc2cc(C)nc(C(C)(F)F)n2)c(-c2cccc([C@H]3CN(C)CCO3)n2)cn1] or 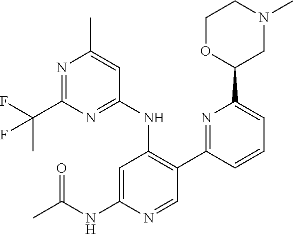 [SMILES: CC(=O)Nc1cc(Nc2cc(C)nc(C(C)(F)F)n2)c(-c2cccc([C@@H]3CN(C)CCO3)n2)cn1] LCMS m/z = 484.2 [M + H]+ 1H NMR (MeOD-d4, 400 MHz): δ (ppm) 9.32 (s, 1H), 8.70 (s, 1H), 7.85-8.05 (m, 2H), 7.49 (d, J = 8.0 Hz, 1H), 7.01 (s, 1H), 4.82 (dd, J = 10.3, 2.3 Hz, 1H), 4.15-4.26 (m, 1H), 3.96 (td, J = 11.8, 2.5 Hz, 1H), 3.18 (dt, J = 11.5, 2.0 Hz, 1H), 2.90 (dd, J = 11.5, 1.5 Hz, 1H), 2.51 (s, 3H), 2.39 (s, 3H), 2.31-2.37 (m, 1H), 2.06-2.26 (m, 7H) |
| 643 | (R)-N-(4′-((2-(1,1-difluoroethyl)-6-methylpyrimidin-4-yl)amino)-5-(4-methylmorpholin-2- yl)-[2,3′-bipyridin]-6′-yl)acetamide or (S)-N-(4′-((2-(1,1-difluoroethyl)-6-methylpyrimidin-4- yl)amino)-5-(4-methylmorpholin-2-yl)-[2,3′-bipyridin]-6′-yl)acetamide 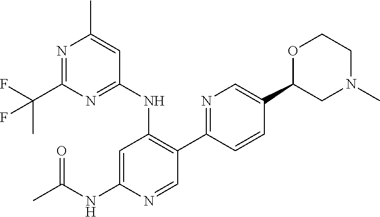 [SMILES: CC(=O)Nc1cc(Nc2cc(C)nc(C(C)(F)F)n2)c(-c2ccc([C@@H]3CN(C)CCO3)cn2)cn1] or 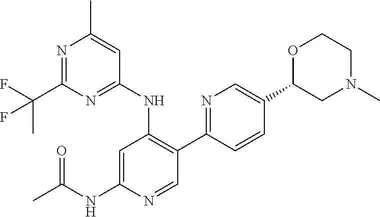 [SMILES: CC(=O)Nc1cc(Nc2cc(C)nc(C(C)(F)F)n2)c(-c2ccc([C@H]3CN(C)CCO3)cn2)cn1] LCMS m/z = 484.2 [M + H]+ 1H NMR (MeOD-d4, 400 MHz): δ (ppm) 9.26 (s, 1H), 8.78 (s, 1H), 8.69 (s, 1H), 7.96 (d, J = 1.5 Hz, 2H), 6.99 (s, 1H), 4.71 (br d, J = 10.0 Hz, 1H), 4.11 (br d, J = 12.0 Hz, 1H), 3.87 (br t, J = 11.5 Hz, 1H), 3.02-3.12 (m, 1H), 2.82-2.97 (m, 1H), 2.49 (s, 3H), 2.44 (br s, 4H), 2.04-2.24 (m,7H) |
| 644 | (S)-N-(4′-((2-(1,1-difluoroethyl)-6-methylpyrimidin-4-yl)amino)-5-(4-methylmorpholin-2- yl)-[2,3′-bipyridin]-6′-yl)acetamide or (R)-N-(4′-((2-(1,1-difluoroethyl)-6-methylpyrimidin- 4-yl)amino)-5-(4-methylmorpholin-2-yl)-[2,3′-bipyridin]-6′-yl)acetamide 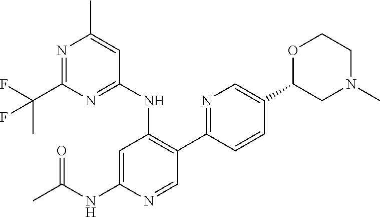 [SMILES: CC(=O)Nc1cc(Nc2cc(C)nc(C(C)(F)F)n2)c(-c2ccc([C@H]3CN(C)CCO3)cn2)cn1] or 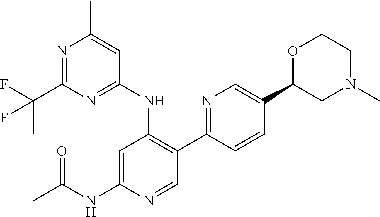 [SMILES: CC(=O)Nc1cc(Nc2cc(C)nc(C(C)(F)F)n2)c(-c2ccc([C@@H]3CN(C)CCO3)cn2)cn1] LCMS m/z = 484.2 [M + H]+ 1H NMR (MeOD-d4, 400 MHz): δ (ppm) 9.27 (s, 1H), 8.77 (s, 1H), 8.69 (s, 1H), 7.95 (s, 2H), 6.98 (s, 1H), 4.68 (dd, J = 10.5, 2.5 Hz, 1H), 4.05-4.12 (m, 1H), 3.85 (td, J = 11.5, 2.5 Hz, 1H), 3.03 (dt, J = 12.0, 2.0 Hz, 1H), 2.77-2.87 (m, 1H), 2.49 (s, 3H), 2.37 (s, 3H), 2.30 (td, J = 11.8, 3.5 Hz, 1H), 2.21 (s, 3H), 2.04-2.17 (m, 4H) |
| 645 | (S)-N-(4′-((2-(1,1-difluoroethyl)-6-methylpyrimidin-4-yl)amino)-5-(4-methylmorpholin-3- yl)-[2,3′-bipyridin]-6′-yl)acetamide or (R)-N-(4′-((2-(1,1-difluoroethyl)-6-methylpyrimidin- 4-yl)amino)-5-(4-methylmorpholin-3-yl)-[2,3′-bipyridin]-6′-yl)acetamide 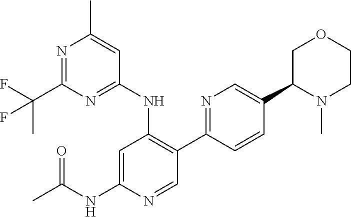 [SMILES: CC(=O)Nc1cc(Nc2cc(C)nc(C(C)(F)F)n2)c(-c2ccc([C@H]3COCCN3C)cn2)cn1] or 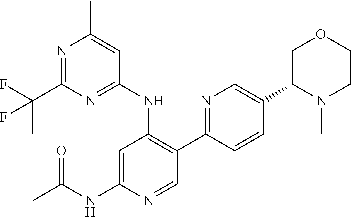 [SMILES: CC(=O)Nc1cc(Nc2cc(C)nc(C(C)(F)F)n2)c(-c2ccc([C@@H]3COCCN3C)cn2)cn1] LCMS m/z = 484.2 [M+H]+; 1H NMR (MeOD-d4, 400 MHz): δ (ppm) 9.27 (s, 1H), 8.76 (t, J = 1.5 Hz, 1H), 8.72 (s, 1H), 7.99 (d, J = 1.5 Hz, 2H), 7.01 (s, 1H), 3.92-4.01 (m, 1H), 3.75- 3.88 (m, 2H), 3.44-3.54 (m, 1H), 3.28-3.31 (m, 1H), 2.90-2.99 (m, 1H), 2.44-2.54 (m, 4H), 2.23 (s, 3H), 2.06-2.19 (m, 6H) |
| 646 | (R)-N-(4′-((2-(1,1-difluoroethyl)-6-methylpyrimidin-4-yl)amino)-5-(4-methylmorpholin-3- yl)-[2,3′-bipyridin]-6′-yl)acetamide or (S)-N-(4′-((2-(1,1-difluoroethyl)-6-methylpyrimidin-4- yl)amino)-5-(4-methylmorpholin-3-yl)-[2,3′-bipyridin]-6′-yl)acetamide 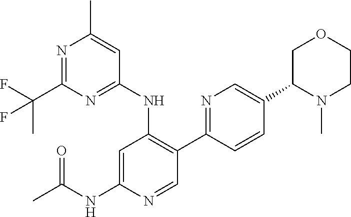 [SMILES: CC(=O)Nc1cc(Nc2cc(C)nc(C(C)(F)F)n2)c(-c2ccc([C@@H]3COCCN3C)cn2)cn1] or 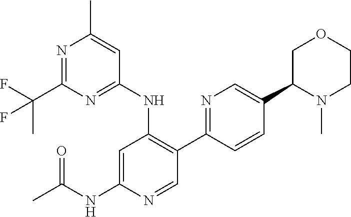 [SMILES: CC(=O)Nc1cc(Nc2cc(C)nc(C(C)(F)F)n2)c(-c2ccc([C@H]3COCCN3C)cn2)cn1] LCMS m/z = 484.2 [M + H]+; 1H NMR (MeOD-d4, 400 MHz): δ (ppm) 9.27 (s, 1H), 8.76 (t, J = 1.5 Hz, 1H), 8.72 (s, 1H), 7.99 (d, J = 1.5 Hz, 2H), 7.01 (s, 1H), 3.92-4.01 (m, 1H), 3.74- 3.88 (m, 2H), 3.44-3.53 (m, 1H), 3.26-3.30 (m, 1H), 2.94 (d, J = 12.0 Hz, 1H), 2.44-2.53 (m, 4H), 2.23 (s, 3H), 2.06-2.19 (m, 6H) |
| 647 | N-(4′-((2-(1,1-difluoroethyl)-6-methylpyrimidin-4-yl)amino)-6-(tetrahydro2H-pyran-2-yl)- [2,3′-bipyridin]-6′-yl)acetamide 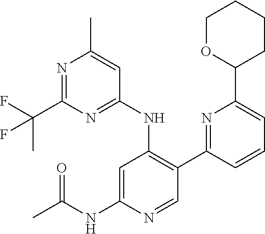 [SMILES: CC(=O)Nc1cc(Nc2cc(C)nc(C(C)(F)F)n2)c(-c2cccc(C3CCCCO3)n2)cn1] LCMS m/z = 469.2 [M + H]+ 1H NMR (MeOD-d4, 400 MHz): δ (ppm) 9.29 (s, 1H), 8.75 (s, 1H), 7.87-8.00 (m, 2H), 7.44 (d, J = 7.0 Hz, 1H), 7.10 (s, 1H), 4.67 (dd, J = 11.0, 2.0 Hz, 1H), 4.25-4.34 (m, 1H), 3.82 (td, J = 11.5, 2.5 Hz, 1H), 2.52 (s, 3H), 2.23 (s, 3H), 2.13 (t, J = 18.8 Hz, 5H), 1.68-1.95 (m, 4H) |
| 648 | N-(4′-((2-(1,1-difluoroethyl)-6-methylpyrimidin-4-yl)amino)-5-((dimethylamino)methyl)- [2,3′-bipyridin]-6′-yl)acetamide 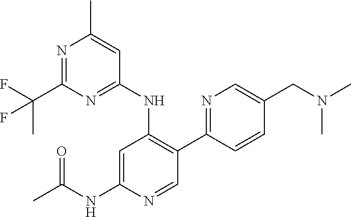 [SMILES: CC(=O)Nc1cc(Nc2cc(C)nc(C(C)(F)F)n2)c(-c2ccc(CN(C)C)cn2)cn1] LCMS m/z = 442.2 [M + H]+ 1H NMR (DMSO-d6, 600 MHz): δ (ppm) 12.27-12.97 (m, 1H), 10.58 (s, 1H), 9.21 (s, 1H), 8.82 (s, 1H), 8.80 (s, 1H), 8.16 (br d, J = 8.4 Hz, 1H), 8.02 (br d, J = 7.6 Hz, 1H), 7.09 (s, 1H), 2.56-2.72 (m, 6H), 2.54-2.55 (m, 2H), 2.44 (s, 3H), 2.03- 2.19 (m, 6H) |
| 649 | N-(4′-((2-(1,1-difluoroethyl)-6-methylpyrimidin-4-yl)amino)-5-(morpholinomethyl)-[2,3′- bipyridin]-6′-yl)acetamide 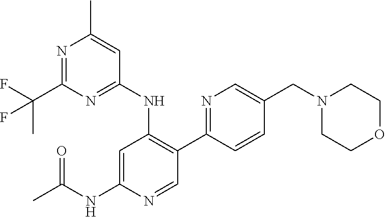 [SMILES: CC(=O)Nc1cc(Nc2cc(C)nc(C(C)(F)F)n2)c(-c2ccc(CN3CCOCC3)cn2)cn1] LCMS m/z = 484.2 [M + H]+ 1H NMR (DMSO-d6, 600 MHz): δ (ppm) 12.89 (s, 1H), 10.53 (s, 1H), 9.27 (s, 1H), 8.80 (s, 1H), 8.72 (d, J = 1.9 Hz, 1H), 8.08 (d, J = 8.0 Hz, 1H), 7.90 (dd, J = 8.2, 2.1 Hz, 1H), 7.12 (s, 1H), 3.55-3.66 (m, 6H), 2.39-2.46 (m, 7H), 2.04-2.18 (m, 6H) |
| 650 | N-(4′-((2-(1,1-difluoroethyl)-6-methylpyrimidin-4-yl)amino)-6-(morpholinomethyl)-[2,3′- bipyridin]-6′-yl)acetamide 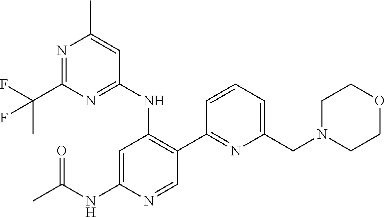 [SMILES: CC(=O)Nc1cc(Nc2cc(C)nc(C(C)(F)F)n2)c(-c2cccc(CN3CCOCC3)n2)cn1] LCMS m/z = 484.2 [M + H]+ 1H NMR (DMSO-d6, 600 MHz): δ (ppm) 13.34 (s, 1H), 10.54 (br s, 1H), 9.26 (br s, 1H), 8.84 (br s, 1H), 8.04 (d, J = 8.0 Hz, 1H), 7.96 (br t, J = 7.6 Hz, 1H), 7.44 (br d, J = 7.2 Hz, 1H), 7.04 (br s, 1H), 3.78 (s, 2H), 3.61 (br t, J = 4.4 Hz, 4H), 2.50-2.52 (m, 4H), 2.44 (br s, 3H), 2.04-2.19 (m, 6H) |
| 651 | N-(4′-((2-(1,1-difluoroethyl)-6-methylpyrimidin-4-yl)amino)-5-(4-methylpiperazin-1-yl)- [2,3′-bipyridin]-6′-yl)acetamide 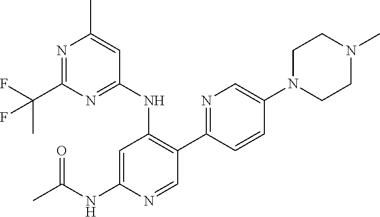 [SMILES: CC(=O)Nc1cc(Nc2cc(C)nc(C(C)(F)F)n2)c(-c2ccc(N3CCN(C)CC3)cn2)cn1] LCMS m/z = 483.4 [M + H]+ 1H NMR (600 MHz, DMSO) δ 13.08 (s, 1H), 10.43 (s, 1H), 8.71 (s, 1H), 8.48 (d, J = 3.0 Hz, 1H), 7.96 (d, J = 9.0 Hz, 1H), 7.54 (dd, J = 9.1, 3.1 Hz, 1H), 7.04 (s, 1H), 3.31 (d, J = 4.8 Hz, 8H), 2.43 (s, 3H), 2.25 (s, 3H), 2.15-2.07 (m, 6H) |
| 652 | N-(4′-((2-(1,1-difluoroethyl)-6-methylpyrimidin-4-yl)amino)-5-(dimethylamino)-[2,3′- bipyridin]-6′-yl)acetamide 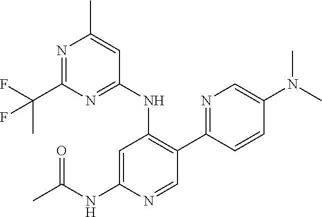 [SMILES: CC(=O)Nc1cc(Nc2cc(C)nc(C(C)(F)F)n2)c(-c2ccc(N(C)C)cn2)cn1] LCMS m/z = 428.1 [M + H]+ |
| 653 | N-(6-(2-cyanopropan-2-yl)-4′-((2-(1,1-difluoroethyl)pyrimidin-4-yl)amino)-[2,3′-bipyridin]- 6′-yl)acetamide 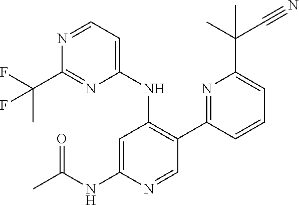 LCMS m/z = 428 [M + H]+ |
| 654 | N-(6-(2-cyanopropan-2-yl)-4′-((2-(1,1-difluoroethyl)-6-methylpyrimidin-4-yl)amino)-[2,3′- bipyridin]-6′-yl)acetamide 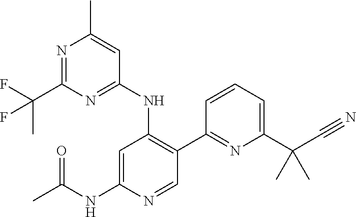 [SMILES: CC(=O)Nc1cc(Nc2cc(C)nc(C(C)(F)F)n2)c(-c2cccc(C(C)(C)C#N)n2)cn1] LCMS m/z = 452 [M + H]+ |
| 655 | N-(5-(5-cyanopyrimidin-2-yl)-4-((2-(1,1-difluoroethyl)-6-methylpyrimidin-4- yl)amino)pyridin-2-yl)acetamide 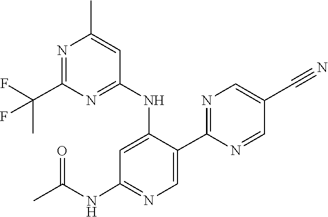 [SMILES: CC(=O)Nc1cc(Nc2cc(C)nc(C(C)(F)F)n2)c(-c2ncc(C#N)cn2)cn1] LCMS m/z = 411.3 [M + H]+ 1H NMR (600 MHz, DMSO) δ 12.31 (s, 1H), 10.75 (s, 1H), 9.40 (s, 1H), 9.36 (s, 1H), 9.32 (s, 1H), 7.18 (s, 1H), 2.47 (s, 3H), 2.15-2.07 (m, 6H). |
| 656 | N-(4-((2-(1,1-difluoroethyl)-6-methylpyrimidin-4-yl)amino)-5-(5-methoxypyrimidin-2- yl)pyridin-2-yl)acetamide 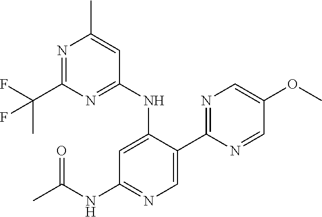 [SMILES: COc1cnc(-c2cnc(NC(C)=O)cc2Nc2cc(C)nc(C(C)(F)F)n2)nc1] LCMS m/z = 416.3 [M + H]+ 1H NMR (600 MHz, DMSO) δ 12.78 (s, 1H), 10.64 (s, 1H), 9.34 (s, 1H), 9.25 (s, 1H), 8.78 (s, 1H), 7.19 (s, 1H), 4.00 (s, 3H), 2.47 (s, 3H), 2.17-2.10 (m, 6H) |
| 657 | N-(5-(4-cyanO6-methylpyrimidin-2-yl)-4-((2-(1,1-difluoroethyl)-6-methylpyrimidin-4- yl)amino)pyridin-2-yl)acetamide 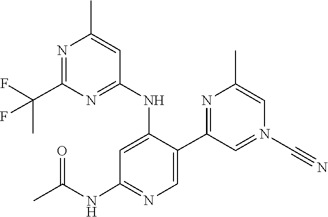 LCMS m/z = 425.1 [M + H]+ 1H NMR (600 MHz, DMSO) δ 12.04 (s, 1H), 10.73 (s, 1H), 9.22 (s, 1H), 9.19 (s, 1H), 8.00 (d, J = 0.7 Hz, 1H), 6.96 (d, J = 1.0 Hz, 1H), 2.70 (s, 3H), 2.48-2.46 (m, 3H), 2.14 (s, 3H), 2.06 (td, J = 19.1, 10.7 Hz, 3H) |
| 658 | N-(4-((2-(1,1-difluoroethyl)-6-methylpyrimidin-4-yl)amino)-5-(4-methoxypyrimidin-2- yl)pyridin-2-yl)acetamide 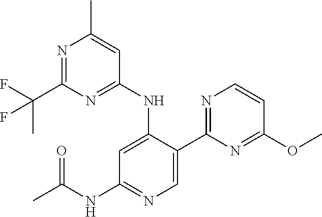 [SMILES: COc1ccnc(-c2cnc(NC(C)=O)cc2Nc2cc(C)nc(C(C)(F)F)n2)n1] LCMS m/z = 416.1 [M + H]+ 1H NMR (600 MHz, DMSO) δ 13.06 (s, 1H), 10.61 (s, 1H), 9.36 (d, J = 8.7 Hz, 1H), 8.72 (d, J = 5.9 Hz, 1H), 7.18 (s, 1H), 6.96 (d, J = 5.9 Hz, 1H), 4.09 (s, 3H), 2.46 (s, 3H), 2.17-2.08 (m, 6H) |
| 659 | N-(4-((2-(1,1-difluoroethyl)-6-methylpyrimidin-4-yl)amino)-5-(5-fluorO4- methoxypyrimidin-2-yl)pyridin-2-yl)acetamide 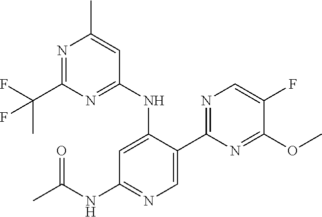 [SMILES: COc1nc(-c2cnc(NC(C)=O)cc2Nc2cc(C)nc(C(C)(F)F)n2)ncc1F] LCMS m/z = 434.1[(M + H]+ 1H NMR (600 MHz, DMSO) δ 12.48 (s, 1H), 10.61 (s, 1H), 9.31 (s, 1H), 9.24 (s, 1H), 8.78 (d, J = 2.8 Hz, 1H), 7.15 (s, 1H), 4.18 (s, 3H), 2.46 (s, 3H), 2.15-2.07 (m, 6H) |
| 660 | N-(4-((2-(1,1-difluoroethyl)-6-methylpyrimidin-4-yl)amino)-5-(5- (difluoromethoxy)pyrimidin-2-yl)pyridin-2-yl)acetamide 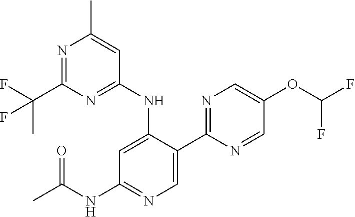 [SMILES: CC(=O)Nc1cc(Nc2cc(C)nc(C(C)(F)F)n2)c(-c2ncc(OC(F)F)cn2)cn1] LCMS m/z = 452.0 [M + H]+ 1H NMR (600 MHz, DMSO) δ 12.49 (s, 1H), 10.65 (s, 1H), 9.40 (s, 1H), 9.28 (s, 1H), 9.00 (s, 1H), 7.60-7.21 (m, 3H), 2.47 (s, 3H), 2.17-2.09 (m, 6H). |
| 661 | N-(4-((2-(1,1-difluoroethyl)-6-methylpyrimidin-4-yl)amino)-5-(2- (methoxymethyl)pyrimidin-4-yl)pyridin-2-yl)acetamide 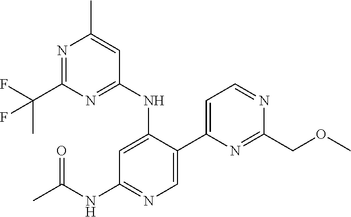 [SMILES: COCc1nccc(-c2cnc(NC(C)=O)cc2Nc2cc(C)nc(C(C)(F)F)n2)n1] LCMS m/z = 430.3 [M + HH]+ 1H NMR (600 MHz, DMSO) δ 13.46 (s, 1H), 10.65 (s, 1H), 9.43 (s, 1H), 9.03 (s, 1H), 8.86 (d, J = 5.6 Hz, 1H), 8.16 (d, J = 5.6 Hz, 1H), 7.02 (s, 1H), 4.78 (d, J = 3.2 Hz, 2H), 3.53 (d, J = 2.7 Hz, 3H), 2.47 (s, 3H), 2.17-2.05 (m, 6H) |
| 662 | N-(4-((2-(1,1-difluoroethyl)-6-methylpyrimidin-4-yl)amino)-5-(5- ((difluoromethoxy)methyl)pyrazin-2-yl)pyridin-2-yl)acetamide 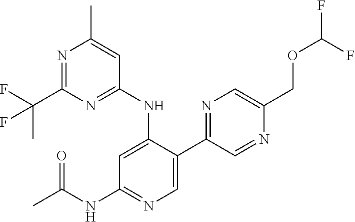 [SMILES: CC(=O)Nc1cc(Nc2cc(C)nc(C(C)(F)F)n2)c(-c2cnc(COC(F)F)cn2)cn1] LCMS m/z = 466.3 [M + H]+ 1H NMR (600 MHz, DMSO) δ 11.29 (s, 1H), 10.63 (s, 1H), 9.20 (d, J = 1.5 Hz, 1H), 9.10 (s, 1H), 8.83 (d, J = 1.5 Hz, 1H), 8.79 (s, 1H), 7.04 (s, 1H), 6.85 (d, J = 75.1 Hz, 1H), 5.12 (s, 2H), 2.41 (s, 3H), 2.13 (s, 3H), 2.02 (t, J = 19.2 Hz, 3H). |
| 663 | N-(4-((2-(1,1-difluoroethyl)-6-methylpyrimidin-4-yl)amino)-5-(4-methoxy-5- methylpyrimidin-2-yl)pyridin-2-yl)acetamide 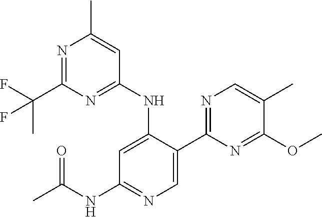 [SMILES: COc1nc(-c2cnc(NC(C)=O)cc2Nc2cc(C)nc(C(C)(F)F)n2)ncc1C] LCMS m/z = 430.3 [M + H]+ 1H NMR (600 MHz, DMSO) δ 13.04 (s, 1H), 10.58 (s, 1H), 9.33 (d, J = 3.0 Hz, 1H), 8.56 (q, J = 0.8 Hz, 1H), 7.15 (d, J = 0.9 Hz, 1H), 4.11 (s, 3H), 2.47- 2.45 (m, 3H), 2.18 (d, J = 0.9 Hz, 3H), 2.16-2.09 (m, 6H) |
| 664 | N-(4-((2-(1,1-difluoroethyl)-6-methylpyrimidin-4-yl)amino)-5-(6-(2-methoxyethyl)pyrazin- 2-yl)pyridin-2-yl)acetamide 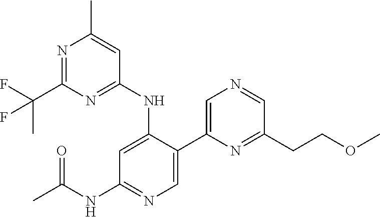 [SMILES: COCCc1cncc(-c2cnc(NC(C)=O)cc2Nc2cc(C)nc(C(C)(F)F)n2)n1] LCMS m/z = 444.2 [M + H]+ |
| 665 | N-(4-((2-(1,1-difluoroethyl)-6-methylpyrimidin-4-yl)amino)-5-(6- (methoxymethyl)pyrimidin-4-yl)pyridin-2-yl)acetamide 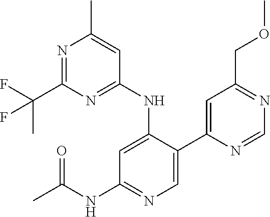 [SMILES: COCc1cc(-c2cnc(NC(C)=O)cc2Nc2cc(C)nc(C(C)(F)F)n2)ncn1] LCMS m/z = 430.2 [M + H]+ 1H NMR (MeOD d4, 400 MHz): δ (ppm) 9.23 (s, 1H), 9.12 (d, J = 1.0 Hz, 1H), 8.72 (s, 1H), 7.95 (s, 1H), 6.94 (s, 1H), 4.51 (s, 2H), 3.44 (s, 3H), 2.41 (s, 3H), 2.11 (s, 3H), 2.00 (t, J = 18.8 Hz, 3H) |
| 666 | N-(5-(4-cyclopropylpyrimidin-2-yl)-4-((2-(1,1-difluoroethyl)-6-methylpyrimidin-4- yl)amino)pyridin-2-yl)acetamide 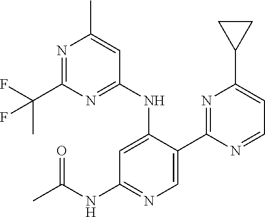 [SMILES: CC(=O)Nc1cc(Nc2cc(C)nc(C(C)(F)F)n2)c(-c2nccc(C3CC3)n2)cn1] LCMS m/z = 426.3 [M + H]+; 1H NMR (MeOD-d4, 400 MHz): δ (ppm) 9.38 (s, 1H), 9.27 (s, 1H), 8.68 (d, J = 5.0 Hz, 1H), 7.31 (d, J = 5.0 Hz, 1H), 7.09 (s, 1H), 2.52 (s, 3H), 2.19-2.27 (m, 4H), 2.06-2.18 (m, 3H), 1.20-1.34 (m, 4H) |
| 667 | N-(5-(6-cyclopropylpyrazin-2-yl)-4-((2-(1,1-difluoroethyl)-6-methylpyrimidin-4- yl)amino)pyridin-2-yl)acetamide 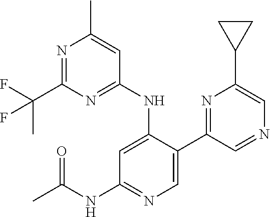 [SMILES: CC(=O)Nc1cc(Nc2cc(C)nc(C(C)(F)F)n2)c(-c2cncc(C3CC3)n2)cn1] LCMS m/z = 426 [M + H]+ |
| 668 | N-(4-((2-(1,1-difluoroethyl)-6-methylpyrimidin-4-yl)amino)-5-(pyrimidin-2-yl)pyridin-2- yl)acetamide 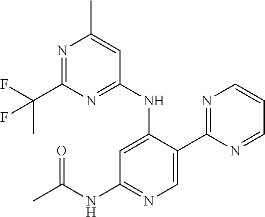 [SMILES: CC(=O)Nc1cc(Nc2cc(C)nc(C(C)(F)F)n2)c(-c2ncccn2)cn1] LCMS m/z = 386.2 [M + H]+ 1H NMR (MeOD-d4, 400 MHz): δ (ppm) 9.42 (d, J = 8.0 Hz, 2H), 8.94 (d, J = 4.5 Hz, 2H), 7.39-7.46 (m, 1H), 7.08 (s, 1H), 2.52 (s, 3H),2.22 (s, 3H), 2.14 (t, J = 19.0 Hz, 3H) |
| 669 | N-(5-(5-chloropyrimidin-2-yl)-4-((2-(1,1-difluoroethyl)-6-methylpyrimidin-4- yl)amino)pyridin-2-yl)acetamide 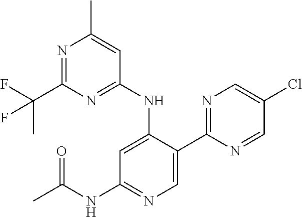 [SMILES: CC(=O)Nc1cc(Nc2cc(C)nc(C(C)(F)F)n2)c(-c2ncc(Cl)cn2)cn1] LCMS m/z = 420.2 [M + H]+ 1H NMR (CDCl₃, 400 MHz): δ (ppm) 9.40 (d, J = 7.0 Hz, 2H), 8.98 (s, 2H), 7.09 (s, 1H), 2.52 (s, 3H), 2.22 (s, 3H), 2.14 (t, J = 18.8 Hz, 3H) |
| 670 | N-(4-((2-(1,1-difluoroethyl)-6-methylpyrimidin-4-yl)amino)-5-(5- (difluoromethyl)pyrimidin-2-yl)pyridin-2-yl)acetamide 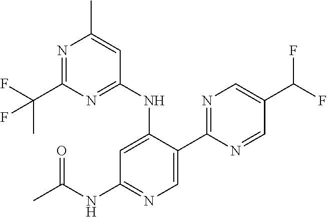 [SMILES: CC(=O)Nc1cc(Nc2cc(C)nc(C(C)(F)F)n2)c(-c2ncc(C(F)F)cn2)cn1] LCMS m/z = 436.2 [M + H]+ 1H NMR (DMSO-d6, 400 MHz): δ (ppm) 12.67 (s, 1H), 10.71 (s, 1H), 9.44 (s, 1H), 9.37 (s, 1H), 9.21-9.26 (m, 2H), 7.11-7.52 (m, 2H), 2.48 (s, 3H), 2.07-2.22 (m, 6H) |
| 671 | N-(4-((2-(1,1-difluoroethyl)-6-methylpyrimidin-4-yl)amino)-5-(4-methylpyrimidin-2- yl)pyridin-2-yl)acetamide 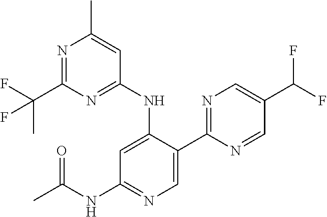 LCMS m/z = 400.2 [M + H]+ 1H NMR (DMSO-d6, 600 MHz): δ (ppm) 13.09 (s, 1H), 10.64 (s, 1H), 9.37 (d, J = 16.4 Hz, 1H), 8.85 (d, J = 5.0 Hz, 1H), 7.41 (d, J = 5.3 Hz, 1H), 7.10 (s, 1H), 2.65 (s, 3H), 2.48 (s, 3H), 2.08-2.21 (m, 6H) |
| 672 | N-(4-((2-(1,1-difluoroethyl)-6-methylpyrimidin-4-yl)amino)-5-(5- (methoxymethyl)pyrimidin-2-yl)pyridin-2-yl)acetamide 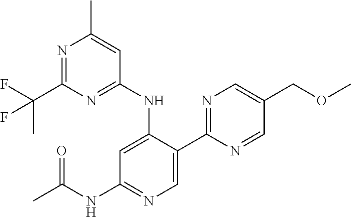 [SMILES: COCc1cnc(-c2cnc(NC(C)=O)cc2Nc2cc(C)nc(C(C)(F)F)n2)nc1] LCMS m/z = 430.2 [M + H]+ 1H NMR (MeOD-d4, 400 MHz): δ (ppm) 9.45 (d, J = 8.0 Hz, 2H), 8.95 (s, 2H), 7.12 (s, 1H), 4.60 (s, 2H), 3.51 (s, 3H), 2.54 (s, 3H), 2.24 (s, 3H), 2.09-2.22 (m, 3H) |
| 673 | N-(4-((2-(1,1-difluoroethyl)-6-methylpyrimidin-4-yl)amino)-5-(5-fluorO4-methylpyrimidin- 2-yl)pyridin-2-yl)acetamide 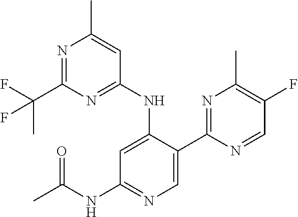 [SMILES: CC(=O)Nc1cc(Nc2cc(C)nc(C(C)(F)F)n2)c(-c2ncc(F)c(C)n2)cn1] LCMS m/z = 418.2 [M + H]+ 1H NMR (MeOH-d4, 400 MHz): δ (ppm) 9.38 (s, 2H), 8.75 (d, J = 2.0 Hz, 1H), 7.06 (s, 1H), 2.70 (d, J = 2.5 Hz, 3H), 2.54 (s, 3H), 2.24 (s, 3H), 2.15 (t, J = 19.0 Hz, 3H) |
| 674 | N-(4-((2-(1,1-difluoroethyl)-6-methylpyrimidin-4-yl)amino)-5-(4-morpholinopyrimidin-2- yl)pyridin-2-yl)acetamide 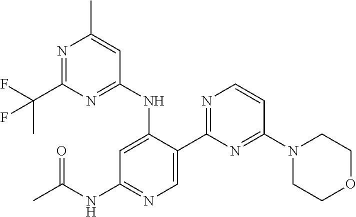 [SMILES: CC(=O)Nc1cc(Nc2cc(C)nc(C(C)(F)F)n2)c(-c2nccc(N3CCOCC3)n2)cn1] LCMS m/z = 471.2 [MH]+ 1H NMR (MeOD-d4, 400 MHz): δ (ppm) 9.28-9.37 (m, 2H), 8.40 (d, J = 6.0 Hz, 1H), 7.06 (s, 1H), 6.77 (d, J = 6.5 Hz, 1H), 3.77-3.89 (m, 8H), 2.53 (s, 3H), 2.23 (s, 3H), 2.07-2.20 (m, 3H) |
| 675 | N-(4-((2-(1,1-difluoroethyl)-6-methylpyrimidin-4-yl)amino)-5-(6- ((dimethylamino)methyl)pyrazin-2-yl)pyridin-2-yl)acetamide 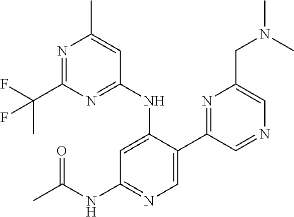 [SMILES: CC(=O)Nc1cc(Nc2cc(C)nc(C(C)(F)F)n2)c(-c2cncc(CN(C)C)n2)cn1] LCMS m/z = 443.2 [M + H]+ 1H NMR (400 MHz, CDCl3) δ ppm: 12.65 (s, 1H), 9.43 (s, 1H), 9.03 (s, 1H), 8.72 (s, 1H), 8.55 (s, 1H), 7.88 (s, 1H), 7.00 (s, 1H), 3.75 (s, 2H), 2.55 (s, 3H), 2.40 (s, 6H), 2.26 (s, 3H), 2.19 (t, J = 18.8 Hz, 3H). |
| 676 | N-(4-((2-(1,1-difluoroethyl)-6-methylpyrimidin-4-yl)amino)-5-(6-methoxypyridazin-3- yl)pyridin-2-yl)acetamide 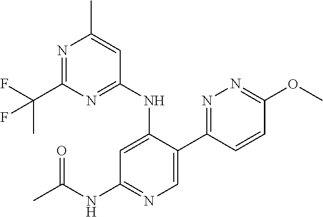 [SMILES: COc1ccc(-c2cnc(NC(C)=O)cc2Nc2cc(C)nc(C(C)(F)F)n2)nn1] LCMS m/z = 416.1 [M + H]+ 1H NMR (600 MHz, DMSO-d₆) δ 11.55 (s, 1H), 10.58 (s, 1H), 9.07 (s, 1H), 8.66 (s, 1H), 8.51 (d, J = 9.3 Hz, 1H), 8.19 (d, J = 9.4 Hz, 1H), 6.95 (s, 1H), 4.10 (d, J = 5.7 Hz, 3H), 2.41 (s, 3H), 2.12 (s, 3H), 2.03 (t, J = 19.2 Hz, 3H) |
| 677 | N-(4-((2-(1,1-difluoroethyl)-6-methylpyrimidin-4-yl)amino)-5-(5-oxO6,7-dihydrO5H- cyclopenta[b]pyridin-2-yl)pyridin-2-yl)acetamide 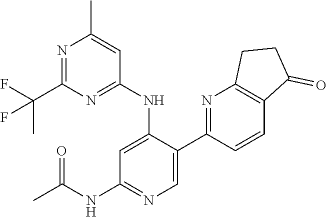 [SMILES: CC(=O)Nc1cc(Nc2cc(C)nc(C(C)(F)F)n2)c(-c2ccc3c(n2)CCC3=O)cn1] LCMS m/z = 439 [M + H]+ |
| 678 | N-(4-((2-(1,1-difluoroethyl)-6-methylpyrimidin-4-yl)amino)-5-(5,7-dihydrofuro[3,4- d]pyrimidin-2-yl)pyridin-2-yl)acetamide 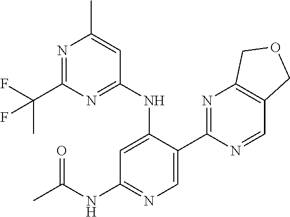 [SMILES: CC(=O)Nc1cc(Nc2cc(C)nc(C(C)(F)F)n2)c(-c2ncc3c(n2)COC3)cn1] LCMS m/z = 428.3 [M + H]+ 1H NMR (600 MHz, DMSO) δ 12.74 (s, 1H), 10.65 (s, 1H), 9.36 (s, 1H), 9.29 (s, 1H), 8.91 (d, J = 1.2 Hz, 1H), 7.11 (s, 1H), 5.20-5.17 (m, 2H), 5.08 (t, J = 1.8 Hz, 2H), 2.45 (s, 3H), 2.17-2.09 (m, 6H) |
| 679 | N-(4-((2-(1,1-difluoroethyl)-6-methylpyrimidin-4-yl)amino)-5-(7,8-dihydrO5H-pyrano[4,3- b]pyridin-2-yl)pyridin-2-yl)acetamide 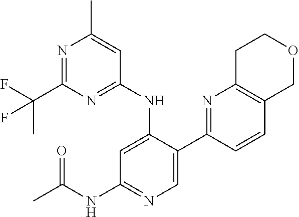 [SMILES: CC(=O)Nc1cc(Nc2cc(C)nc(C(C)(F)F)n2)c(-c2ccc3c(n2)CCOC3)cn1] LCMS m/z = 441.3 [M + H]+ 1H NMR (600 MHz, DMSO) δ 12.90 (s, 1H), 10.57 (s, 1H), 9.22 (s, 1H), 8.76 (s, 1H), 7.90 (d, J = 8.3 Hz, 1H), 7.68 (d, J = 8.2 Hz, 1H), 6.90 (s, 1H), 4.77 (s, 2H), 4.07 (t, J = 5.8 Hz, 2H), 3.12 (t, J = 5.8 Hz, 2H), 2.44 (s, 3H), 2.15-2.05 (m, 6H). |
| 680 | N-(4-((2-(1,1-difluoroethyl)-6-methylpyrimidin-4-yl)amino)-5-(imidazo[1,2-b]pyridazin-6- yl)pyridin-2-yl)acetamide 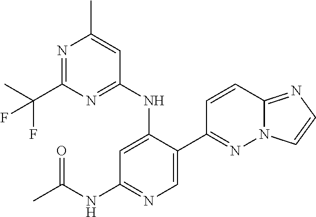 [SMILES: CC(=O)Nc1cc(Nc2cc(C)nc(C(C)(F)F)n2)c(-c2ccc3nccn3n2)cn1] LCMS m/z = 425 [M + H]+ |
| 681 | N-(4-((2-(1,1-difluoroethyl)-6-methylpyrimidin-4-yl)amino)-5-(imidazo[1,2-a]pyrazin-6- yl)pyridin-2-yl)acetamide 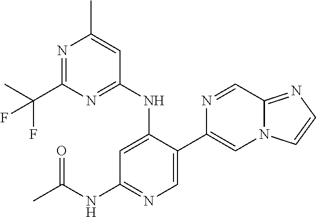 [SMILES: CC(=O)Nc1cc(Nc2cc(C)nc(C(C)(F)F)n2)c(-c2cn3ccnc3cn2)cn1] LCMS m/z = 425 [M + H]+ |
| 682 | N-(4-((2-(1,1-difluoroethyl)-6-methylpyrimidin-4-yl)amino)-5-(7-oxO4a,5,7,7a- tetrahydrofuro[3,4-d]pyrimidin-2-yl)pyridin-2-yl)acetamide 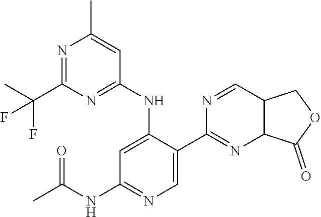 LCMS m/z = 441 [M + H]+ |
| 683 | N-(4-((2-(1,1-difluoroethyl)-6-methylpyrimidin-4-yl)amino)-5-(4,5-dimethyl-4,5,6,7- tetrahydropyrazolo[1,5-a]pyrazin-2-yl)pyridin-2-yl)acetamide 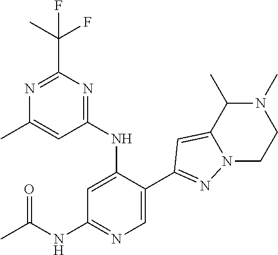 LCMS m/z = 457.4 [M + H]+ |
| 684 | N-(4-((2-(1,1-difluoroethyl)-6-methylpyrimidin-4-yl)amino)-5-(5-methyl-5,6-dihydrO4H- pyrrolo[3,4-d]thiazol-2-yl)pyridin-2-yl)acetamide 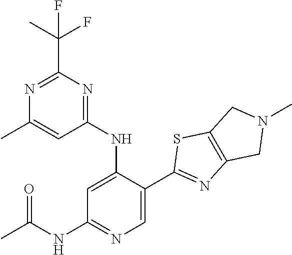 [SMILES: CC(=O)Nc1cc(Nc2cc(C)nc(C(C)(F)F)n2)c(-c2nc3c(s2)CN(C)C3)cn1] LCMS m/z = 446 [M + H]+ |
| 685 | N-(4-((2-(1,1-difluoroethyl)pyrimidin-4-yl)amino)-5-(pyrimidin-2-yl)pyridin-2-yl)acetamide 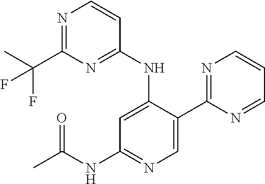 [SMILES: CC(=O)Nc1cc(Nc2ccnc(C(C)(F)F)n2)c(-c2ncccn2)cn1] LCMS m/z = 372.2 [M + H]+ 1H NMR (DMSO-d6, 600 MHz): δ (ppm) 12.96 (s, 1H), 10.67 (s, 1H), 9.33-9.45 (m, 1H), 9.02 (d, J = 4.6 Hz, 1H), 8.64 (d, J = 6.1 Hz, 1H), 7.54 (t, J = 5.0 Hz, 1H), 7.35 (d, J = 5.7 Hz, 1H), 2.08-2.20 (m, 6H) |
| 686 | N-(4-((2-(1,1-difluoroethyl)pyrimidin-4-yl)amino)-5-(5,6-dimethoxypyrazin-2-yl)pyridin-2- yl)acetamide 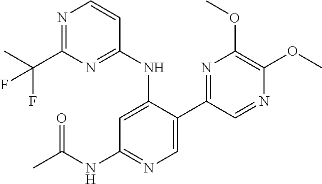 [SMILES: COc1ncc(-c2cnc(NC(C)=O)cc2Nc2ccnc(C(C)(F)F)n2)nc1OC] LCMS m/z = 432 [M + H]+ |
| 687 | N-(4-((2-(1,1-difluoroethyl)pyrimidin-4-yl)amino)-5-(2,6-dimethoxypyrimidin-4-yl)pyridin- 2-yl)acetamide 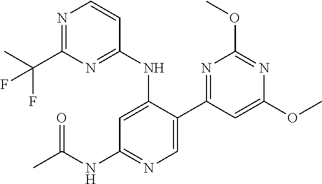 [SMILES: COc1cc(-c2cnc(NC(C)=O)cc2Nc2ccnc(C(C)(F)F)n2)nc(OC)n1] LCMS m/z = 432 [M + H]+ |
| 688 | N-(5-(6-(cyanomethyl)pyrazin-2-yl)-4-((2-(1,1-difluoroethyl)pyrimidin-4-yl)amino)pyridin- 2-yl)acetamide 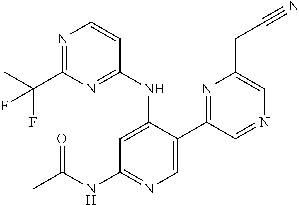 [SMILES: CC(=O)Nc1cc(Nc2ccnc(C(C)(F)F)n2)c(-c2cncc(CC#N)n2)cn1] LCMS m/z = 411.1 [M + H]+ 1H NMR (600 MHz, DMSO) δ 10.97 (s, 1H), 10.67 (s, 1H), 9.13 (s, 1H), 9.12 (s, 1H), 8.76 (s, 1H), 8.63 (s, 1H), 8.55 (d, J = 5.8 Hz, 1H), 7.18 (d, J = 5.8 Hz, 1H), 4.45 (s, 2H), 2.14 (s, 3H), 2.03 (t, J = 19.1 Hz, 3H). |
| 689 | N-(4-((2-(1,1-difluoroethyl)pyrimidin-4-yl)amino)-5-(6-(difluoromethoxy)pyrazin-2- yl)pyridin-2-yl)acetamide 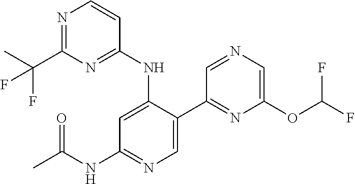 [SMILES: CC(=O)Nc1cc(Nc2ccnc(C(C)(F)F)n2)c(-c2cncc(OC(F)F)n2)cn1] LCMS m/z = 438.2 [M + H]+ 1H NMR (600 MHz, DMSO) δ 10.69 (s, 1H), 10.04 (s, 1H), 8.81 (s, 1H), 8.80 (s, 1H), 8.62 (s, 1H), 8.50-8.48 (m, 1H), 7.72 (s, 1H), 7.18-6.99 (m, 1H), 6.97 (d, J = 5.8 Hz, 1H), 2.14 (s, 3H), 1.93 (t, J = 19.1 Hz, 3H) |
| 690 | N-(4-((2-(1,1-difluoroethyl)pyrimidin-4-yl)amino)-5-(6-(oxetan-3-yloxy)pyrazin-2- yl)pyridin-2-yl)acetamide 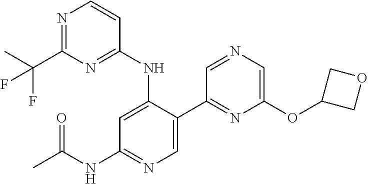 [SMILES: CC(=O)Nc1cc(Nc2ccnc(C(C)(F)F)n2)c(-c2cncc(OC3COC3)n2)cn1] LCMS m/z = 444.2 [M + H]+ 1H NMR (600 MHz, DMSO) δ 10.64 (s, 1H), 9.95 (s, 1H), 8.76 (s, 1H), 8.57 (s, 1H), 8.50 (s, 2H), 8.33 (s, 1H), 7.02 (d, J = 5.8 Hz, 1H), 5.56-5.51 (m, 1H), 4.78-4.74 (m, 2H), 4.59 (ddd, J = 7.5, 5.0, 1.0 Hz, 2H), 2.13 (s, 3H), 1.96 (t, J = 19.0 Hz, 3H). |
| 691 | N-(4-((2-(1,1-difluoroethyl)pyrimidin-4-yl)amino)-5-(6-(2-methoxyethoxy)pyrazin-2- yl)pyridin-2-yl)acetamide 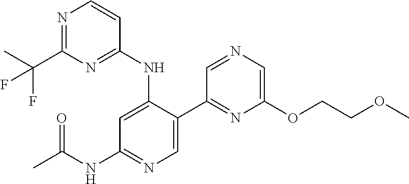 [SMILES: COCCOc1cncc(-c2cnc(NC(C)=O)cc2Nc2ccnc(C(C)(F)F)n2)n1] LCMS m/z = 446.3 [M + H]+ 1H NMR (600 MHz, DMSO) δ 10.66 (s, 1H), 10.28 (s, 1H), 8.82 (s, 1H), 8.61 (s, 1H), 8.57 (s, 1H), 8.51 (d, J = 5.8 Hz, 1H), 8.27 (s, 1H), 7.04 (d, J = 5.9 Hz, 1H), 4.44-4.42 (m, 2H), 3.69-3.66 (m, 2H), 3.28 (s, 3H), 2.13 (s, 3H), 1.97 (t, J = 19.1 Hz, 3H). |
| 692 | N-(4-((2-(1,1-difluoroethyl)pyrimidin-4-yl)amino)-5-(5-((difluoromethoxy)methyl)pyrazin- 2-yl)pyridin-2-yl)acetamide 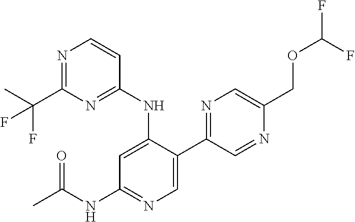 [SMILES: CC(=O)Nc1cc(Nc2ccnc(C(C)(F)F)n2)c(-c2cnc(COC(F)F)cn2)cn1] LCMS m/z = 452.2 [M + H]+ 1H NMR (600 MHz, DMSO) δ 11.19 (s, 1H), 10.66 (s, 1H), 9.16 (d, J = 1.5 Hz, 1H), 9.04 (s, 1H), 8.81 (d, J = 1.5 Hz, 1H), 8.77 (s, 1H), 8.53 (d, J = 5.8 Hz, 1H), 7.16 (d, J = 5.8 Hz, 1H), 6.91 (t, J = 75.1 Hz, 1H), 5.11 (s, 2H), 2.14 (s, 3H), 2.01 (t, J = 19.1 Hz, 3H). |
| 693 | N-(4-((2-(1,1-difluoroethyl)pyrimidin-4-yl)amino)-5-(5-methylpyrazin-2-yl)pyridin-2- yl)acetamide 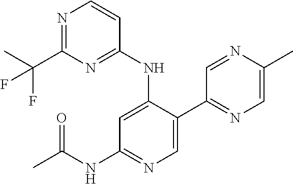 [SMILES: CC(=O)Nc1cc(Nc2ccnc(C(C)(F)F)n2)c(-c2cnc(C)cn2)cn1] LCMS m/z = 386.1 [M + H]+ 1H NMR (500 MHz, CDCl3) δ 12.80 (s, 1H), 12.66 (s, 1H), 9.94 (s, 1H), 9.02 (s, 1H), 8.73 (d, J = 4.9 Hz, 1H), 8.57 (s, 1H), 8.52 (s, 1H), 7.01 (d, J = 5.3 Hz, 1H), 2.72 (s, 3H), 2.37 (s, 3H), 2.18 (t, J = 18.8 Hz, 3H) |
| 694 | N-(5-(5-cyclopropylpyrazin-2-yl)-4-((2-(1,1-difluoroethyl)pyrimidin-4-yl)amino)pyridin-2- yl)acetamide 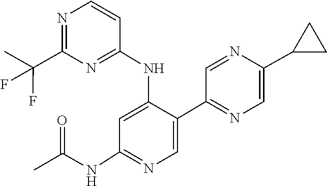 [SMILES: CC(=O)Nc1cc(Nc2ccnc(C(C)(F)F)n2)c(-c2cnc(C3CC3)cn2)cn1] LCMS m/z = 412.1 [M + H]+ 1H NMR (500 MHz, DMSO) δ 11.10 (s, 1H), 10.60 (s, 1H), 9.01 (s, 1H), 8.95 (d, J = 1.5 Hz, 1H), 8.69-8.66 (m, 2H), 8.52 (d, J = 5.9 Hz, 1H), 7.11 (d, J = 5.9 Hz, 1H), 2.27 (ddt, J = 12.9, 8.3, 4.8 Hz, 1H), 2.13 (s, 3H), 2.02 (t, J = 19.1 Hz, 3H), 1.08 (dt, J = 8.1, 3.2 Hz, 2H), 1.00-0.97 (m, 2H). |
| 695 | N-(5-(5-(1,1-difluoroethyl)pyrazin-2-yl)-4-((2-(1,1-difluoroethyl)pyrimidin-4- yl)amino)pyridin-2-yl)acetamide 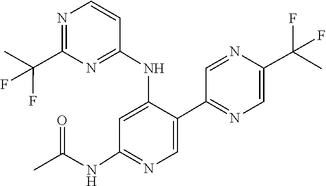 [SMILES: CC(=O)Nc1cc(Nc2ccnc(C(C)(F)F)n2)c(-c2cnc(C(C)(F)F)cn2)cn1] LCMS m/z = 436.1 [M + H]+ 1H NMR (600 MHz, DMSO) δ 11.06 (s, 1H), 10.68 (s, 1H), 9.21 (d, J = 1.5 Hz, 1H), 9.04-8.99 (m, 2H), 8.78 (s, 1H), 8.53 (d, J = 5.8 Hz, 1H), 7.14 (d, J = 5.8 Hz, 1H), 2.14 (s, 3H), 2.02 (dt, J = 40.9, 19.1 Hz, 6H) |
| 696 | N-(4-((2-(1,1-difluoroethyl)pyrimidin-4-yl)amino)-5-(2-(methoxymethyl)pyrimidin-4- yl)pyridin-2-yl)acetamide 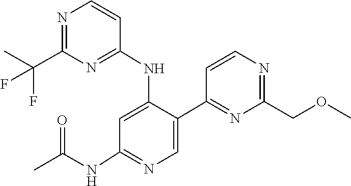 [SMILES: COCc1nccc(-c2cnc(NC(C)=O)cc2Nc2ccnc(C(C)(F)F)n2)n1] LCMS m/z = 416.2 [M + H]+ |
| 697 | N-(5-(5-chloropyrimidin-2-yl)-4-((2-(1,1-difluoroethyl)pyrimidin-4-yl)amino)pyridin-2- yl)acetamide 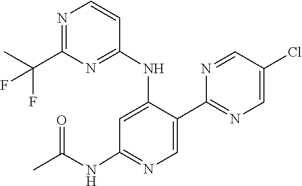 [SMILES: CC(=O)Nc1cc(Nc2ccnc(C(C)(F)F)n2)c(-c2ncc(Cl)cn2)cn1] LCMS m/z = 406.2 [M + H]+ 1H NMR (DMSO-d6, 600 MHz): δ (ppm) 12.31 (s, 1H), 10.70 (s, 1H), 9.34 (s, 1H), 9.25 (s, 1H), 9.10 (s, 1H), 8.64 (d, J = 5.7 Hz, 1H), 7.31 (d, J = 5.7 Hz, 1H), 2.07-2.18 (m, 6H) |
| 698 | N-(4′-((2-(1,1-difluoroethyl)pyrimidin-4-yl)amino)-3-fluorO[2,3′-bipyridin]-6′-yl)acetamide 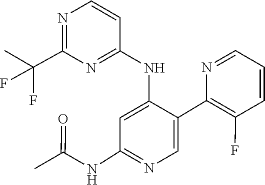 [SMILES: CC(=O)Nc1cc(Nc2ccnc(C(C)(F)F)n2)c(-c2ncccc2F)cn1] LCMS m/z = 389.2 [M + H]+ 1H NMR (DMSO-d6, 600 MHz): δ (ppm) 10.64 (s, 1H), 10.24 (s, 1H), 8.83 (s, 1H), 8.50-8.56 (m, 1H), 8.39-8.48 (m, 1H), 7.83 (ddd, J = 10.6, 8.5, 1.1 Hz, 1H), 7.50 (dt, J = 8.4, 4.2 Hz, 1H), 6.95-7.03 (m, 1H), 2.49-2.53 (m, 3H), 2.14 (s, 3H), 1.93-2.03 (m, 3H) |
| 699 | N-(4′-((2-(1,1-difluoroethyl)pyrimidin-4-yl)amino)-5-fluorO4-methoxy-[2,3′-bipyridin]-6′- yl)acetamide 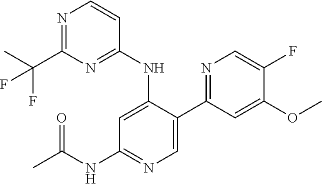 [SMILES: COc1cc(-c2cnc(NC(C)=O)cc2Nc2ccnc(C(C)(F)F)n2)ncc1F] LCMS m/z = 419.2 [M + H]+ 1H NMR (DMSO-d6, 600 MHz): δ (ppm) 12.22 (s, 1H), 10.57 (s, 1H), 9.19 (s, 1H), 8.81 (s, 1H), 8.61 (d, J = 3.1 Hz, 1H), 8.56 (d, J = 5.7 Hz, 1H), 7.78 (d, J = 6.9 Hz, 1H), 7.18 (d, J = 6.1 Hz, 1H), 4.06 (s, 3H), 2.14 (s, 3H), 2.05-2.12 (m, 3H) |
| 700 | N-(4′-((2-(1,1-difluoroethyl)pyrimidin-4-yl)amino)-4-methyl-[2,3′-bipyridin]-6′-yl)acetamide 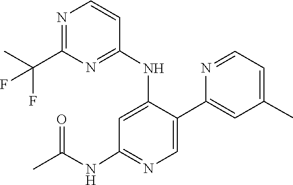 [SMILES: CC(=O)Nc1cc(Nc2ccnc(C(C)(F)F)n2)c(-c2cc(C)ccn2)cn1] LCMS m/z = 385.2 [M + H]+ 1H NMR (MeOD-d4, 400 MHz): δ (ppm) 9.30 (s, 1H), 8.71 (s, 1H), 8.60 (d, J = 5.0 Hz, 1H), 8.48 (d, J = 6.0 Hz, 1H), 7.83 (s, 1H), 7.28 (dd, J = 4.8, 1.3 Hz, 1H), 7.10 (d, J = 6.0 Hz, 1H), 2.49 (s, 3H), 2.23 (s, 3H), 2.13 (t, J = 19.0 Hz, 3H) |
| 701 | N-(5-cyanO4′-((2-(1, 1-difluoroethyl)pyrimidin-4-yl)amino)-4-methyl-[2,3′-bipyridin]-6′- yl)acetamide 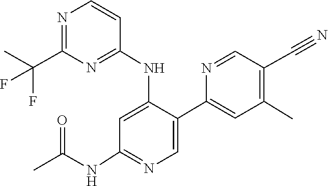 [SMILES: CC(=O)Nc1cc(Nc2ccnc(C(C)(F)F)n2)c(-c2cc(C)c(C#N)cn2)cn1] LCMS m/z = 410.2 [M + H]+ 1H NMR (DMSO-d6, 600 MHz): δ (ppm) 12.00 (s, 1H), 10.68 (s, 1H), 9.17 (s, 1H), 9.05 (s, 1H), 8.81 (s, 1H), 8.58 (d, J = 5.7 Hz, 1H), 8.18 (s, 1H), 7.20 (d, J = 5.7 Hz, 1H), 2.57 (s, 3H), 2.14 (s, 3H), 2.06 (t, J = 19.3 Hz, 3H) |
| 702 | N-(5-(((1R,5S)-6-oxa-3-azabicyclo[3.1.1]heptan-3-yl)methyl)-4′-((2-(1,1- difluoroethyl)pyrimidin-4-yl)amino)-[2,3′-bipyridin]-6′-yl)acetamide 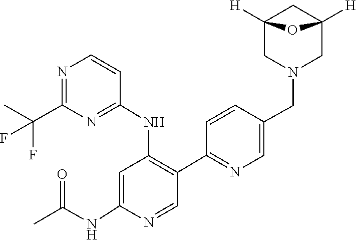 [SMILES: CC(=O)Nc1cc(Nc2ccnc(C(C)(F)F)n2)c(-c2ccc(CN3C[C@H]4C[C@@H](C3)O4)cn2)cn1] LCMS m/z = 482 [M + H]+ |
| 703 | N-(5-((2-oxa-6-azaspiro[3.4]octan-6-yl)methyl)-4′-((2-(1,1-difluoroethyl)pyrimidin-4- yl)amino)-[2,3′-bipyridin]-6′-yl)acetamide 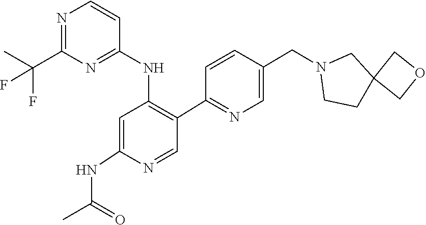 [SMILES: CC(=O)Nc1cc(Nc2ccnc(C(C)(F)F)n2)c(-c2ccc(CN3CCC4(COC4)C3)cn2)cn1] LCMS m/z = 496 [M + H]+ |
| 704 | N-(5-(1-(cyclopropylmethyl)-1H-pyrazol-3-yl)-4-((2-(1,1-difluoroethyl)pyrimidin-4- yl)amino)pyridin-2-yl)acetamide 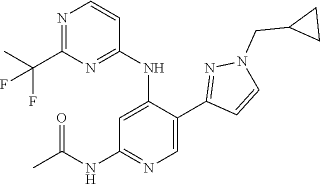 [SMILES: CC(=O)Nc1cc(Nc2ccnc(C(C)(F)F)n2)c(-c2ccn(CC3CC3)n2)cn1] LCMS m/z = 414 [M + H]+ |
| 705 | N-(4-((2-(1,1-difluoroethyl)pyrimidin-4-yl)amino)-5-(1-propyl-1H-pyrazol-3-yl)pyridin-2- yl)acetamide 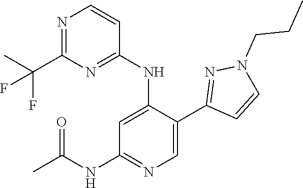 [SMILES: CCCn1ccc(-c2cnc(NC(C)=O)cc2Nc2ccnc(C(C)(F)F)n2)n1] LCMS m/z = 402 [M + H]+ |
| 706 | N-(4-((2-(1,1-difluoroethyl)pyrimidin-4-yl)amino)-5-(1-ethyl-1H-pyrazol-3-yl)pyridin-2- yl)acetamide 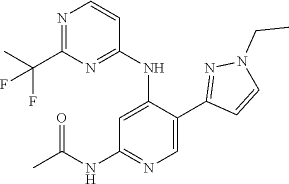 [SMILES: CCn1ccc(-c2cnc(NC(C)=O)cc2Nc2ccnc(C(C)(F)F)n2)n1] LCMS m/z = 388 [M + H]+ |
| 707 | N-(5-(2-cyanO1-methyl-1H-imidazol-4-yl)-4-((2-(1,1-difluoroethyl)pyrimidin-4- yl)amino)pyridin-2-yl)acetamide 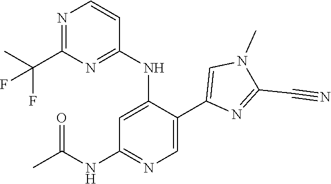 [SMILES: CC(=O)Nc1cc(Nc2ccnc(C(C)(F)F)n2)c(-c2cn(C)c(C#N)n2)cn1] LCMS m/z = 399 [M + H]+ |
| 708 | N-(4-((2-(1,1-difluoroethyl)pyrimidin-4-yl)amino)-5-(2-isopropyl-2H-1,2,3-triazol-4- yl)pyridin-2-yl)acetamide 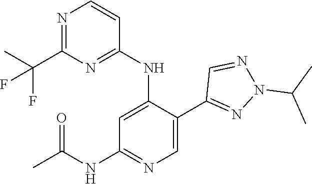 LCMS m/z = 403 [M + H]+ |
| 709 | N-(4-((2-(1,1-difluoroethyl)pyrimidin-4-yl)amino)-5-(2-ethyl-2H-1,2,3-triazol-4-yl)pyridin- 2-yl)acetamide 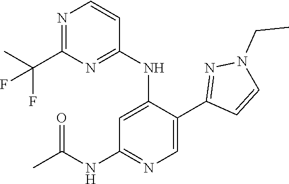 [SMILES: CCn1ccc(-c2cnc(NC(C)=O)cc2Nc2ccnc(C(C)(F)F)n2)n1] LCMS m/z = 389.1 [M + H]+ 1H NMR (600 MHz, DMSO) δ 10.58 (s, 1H), 10.28 (s, 1H), 9.05 (s, 1H), 8.69 (s, 1H), 8.55 (d, J = 5.8 Hz, 1H), 8.28 (s, 1H), 7.13 (d, J = 5.8 Hz, 1H), 4.54 (q, J = 7.3 Hz, 2H), 2.12 (s, 3H), 2.04 (t, J = 19.1 Hz, 3H), 1.50 (t, J = 7.3 Hz, 2H). |
| 710 | N-(4-((2-(1,1-difluoroethyl)pyrimidin-4-yl)amino)-5-(2-(2-methoxyethyl)-2H-1,2,3-triazol- 4-yl)pyridin-2-yl)acetamide 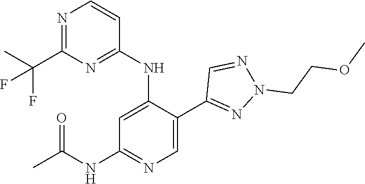 LCMS m/z = 419 [M + H]+ |
| 711 | N-(4-((2-(1,1-difluoroethyl)pyrimidin-4-yl)amino)-5-(2-(methoxymethyl)-2H-1,2,3-triazol- 4-yl)pyridin-2-yl)acetamide 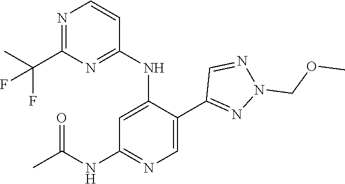 LCMS m/z = 405 [M + H]+ |
| 712 | N-(4-((2-(1,1-difluoroethyl)pyrimidin-4-yl)amino)-5-(2-methoxythiazol-4-yl)pyridin-2- yl)acetamide 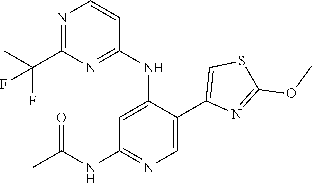 [SMILES: COc1nc(-c2cnc(NC(C)=O)cc2Nc2ccnc(C(C)(F)F)n2)cs1] LCMS m/z = 407 [M + H]+ |
| 713 | N-(4-((2-(1,1-difluoroethyl)pyrimidin-4-yl)amino)-5-(2-(2-fluoropropan-2-yl)thiazol-4- yl)pyridin-2-yl)acetamide 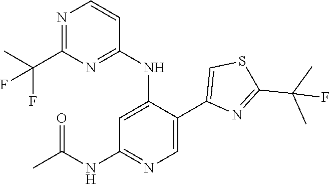 [SMILES: CC(=O)Nc1cc(Nc2ccnc(C(C)(F)F)n2)c(-c2csc(C(C)(C)F)n2)cn1] LCMS m/z = 437 [M + H]+ |
| 714 | N-(4-((2-(1,1-difluoroethyl)pyrimidin-4-yl)amino)-5-(5-ethyl-1,3,4-thiadiazol-2-yl)pyridin- 2-yl)acetamide 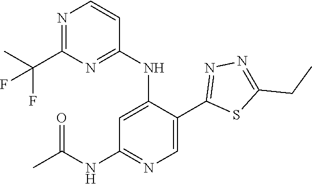 [SMILES: CCc1nnc(-c2cnc(NC(C)=O)cc2Nc2ccnc(C(C)(F)F)n2)s1] LCMS m/z = 406 [M + H]+ |
| 715 | N-(5-(5-(tert-butyl)-1,3,4-thiadiazol-2-yl)-4-((2-(1,1-difluoroethyl)pyrimidin-4- yl)amino)pyridin-2-yl)acetamide 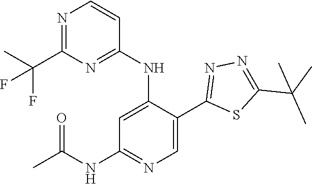 LCMS m/z = 434 [M + H]+ |
| 716 | N-(4-((2-(1,1-difluoroethyl)pyrimidin-4-yl)amino)-5-(3-methyl-1,2,4-thiadiazol-5- yl)pyridin-2-yl)acetamide 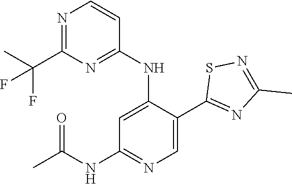 [SMILES: CC(=O)Nc1cc(Nc2ccnc(C(C)(F)F)n2)c(-c2nc(C)ns2)cn1] LCMS m/z = 392 [M + H]+ |
| 717 | N-(5-(5-cyclopropyl-1,3,4-oxadiazol-2-yl)-4-((2-(1,1-difluoroethyl)pyrimidin-4- yl)amino)pyridin-2-yl)acetamide 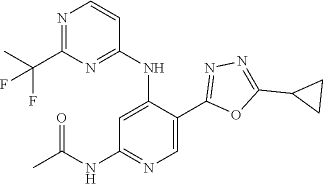 [SMILES: CC(=O)Nc1cc(Nc2ccnc(C(C)(F)F)n2)c(-c2nnc(C3CC3)o2)cn1] LCMS m/z = 402 [M + H]+ |
| 718 | N-(4-((2-(1,1-difluoroethyl)pyrimidin-4-yl)amino)-5-(5-oxO6,7-dihydrO5H- cyclopenta[b]pyridin-2-yl)pyridin-2-yl)acetamide 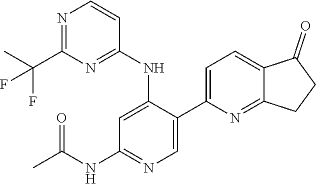 [SMILES: CC(=O)Nc1cc(Nc2ccnc(C(C)(F)F)n2)c(-c2ccc3c(n2)CCC3=O)cn1] LCMS m/z = 425 [M + H]+ |
| 719 | N-(4-((2-(1,1-difluoroethyl)-6-ethylpyrimidin-4-yl)amino)-5-(pyrimidin-2-yl)pyridin-2- yl)acetamide 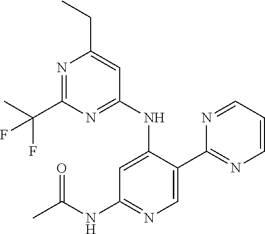 [SMILES: CCc1cc(Nc2cc(NC(C)=O)ncc2-c2ncccn2)nc(C(C)(F)F)n1] LCMS m/z = 400.2 [M + H]+ 1H NMR (MeOD-d4, 400 MHz): δ (ppm) 9.37 (d, J = 15.0 Hz, 2H), 8.92 (d, J = 5.0 Hz, 2H), 7.39 (t, J = 5.0 Hz, 1H), 7.02 (s, 1H), 2.79 (q, J = 7.5 Hz, 2H), 2.21 (s, 3H), 2.13 (t, J = 18.8 Hz, 3H), 1.31-1.36 (m, 3H) |
| 720 | N-(4-((2-(1,1-difluoroethyl)-6-ethylpyrimidin-4-yl)amino)-5-(2-(methoxymethyl)pyrimidin- 4-yl)pyridin-2-yl)acetamide 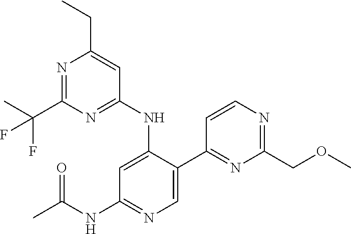 [SMILES: CCc1cc(Nc2cc(NC(C)=O)ncc2-c2ccnc(COC)n2)nc(C(C)(F)F)n1] LCMS m/z = 444.2 [M + H]+ 1H NMR (500 MHz, DCM) δ 14.37 (s, 1H), 12.63 (s, 1H), 10.12 (d, J = 2.5 Hz, 1H), 8.90 (d, J = 5.6 Hz, 1H), 8.61 (d, J = 2.5 Hz, 1H), 7.69 (d, J = 5.5 Hz, 1H), 7.21 (d, J = 2.4 Hz, 1H), 4.87 (d, J = 2.4 Hz, 2H), 3.62 (d, J = 2.4 Hz, 3H), 2.87 (dd, J = 8.8, 6.4 Hz, 2H), 2.33 (d, J = 2.5 Hz, 3H), 2.17 (td, J = 19.0, 2.5 Hz, 3H), 1.39-1.35 (m, 3H) |
| 721 | N-(4-((2-(1,1-difluoroethyl)-6-ethylpyrimidin-4-yl)amino)-5-(5-methyl-4,5,6,7- tetrahydropyrazolo[1,5-a]pyrazin-2-yl)pyridin-2-yl)acetamide 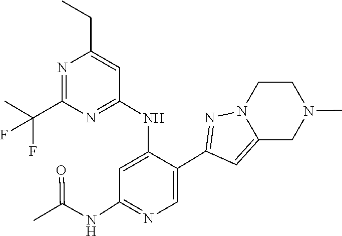 [SMILES: CCc1cc(Nc2cc(NC(C)=O)ncc2-c2cc3n(n2)CCN(C)C3)nc(C(C)(F)F)n1] LCMS m/z = 457.4 [M + H]+ 1H NMR (500 MHz, DMSO-d6) δ ppm 11.23 (br s, 1 H), 10.49 (s, 1 H), 9.28 (s, 1 H), 8.66 (s, 1 H), 7.06 (s, 1 H), 6.91 (br s, 1 H), 4.35-4.74 (m, 4 H), 2.95 (br s, 2 H), 2.73 (q, J = 7.6 Hz, 2H), 2.12 (t, J = 20 Hz, 3 H), 2.11 (s, 3 H), 1.26 (t, J = 7.6 Hz, 3 H). |
| 722 | N-(4′-((5-(2-fluoropropan-2-yl)pyridin-3-yl)amino)-5-(2-hydroxypropan-2-yl)-[2,3′- bipyridin]-6′-yl)acetamide 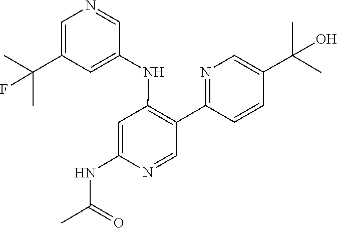 [SMILES: CC(=O)Nc1cc(Nc2cncc(C(C)(C)F)c2)c(-c2ccc(C(C)(C)O)cn2)cn1] LCMS m/z = 424 [M + H]+ 1H NMR (500 MHz, CD2Cl2) δ 12.11 (s, 1H), 8.78 (d, J = 2.5 Hz, 1H), 8.47 (d, J = 2.9 Hz, 2H), 8.41 (d, J = 2.0 Hz, 1H), 8.14 (s, 1H), 7.97 (dd, J = 8.5, 2.5 Hz, 1H), 7.76 (d, J = 8.3 Hz, 2H), 7.24 (t, J = 7.5 Hz, 1H), 7.18-7.12 (m, 1H), 2.34 (s, 1H), 2.17 (s, 3H), 1.78 (s, 3H), 1.73 (s, 3H), 1.62 (s, 6H). |
| 723 | N-(4-((2-(2-fluoropropan-2-yl)-6-methylpyrimidin-4-yl)amino)-5-(5-fluoropyrimidin-2- yl)pyridin-2-yl)acetamide 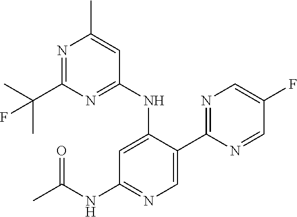 [SMILES: CC(=O)Nc1cc(Nc2cc(C)nc(C(C)(C)F)n2)c(-c2ncc(F)cn2)cn1] LCMS m/z = 400.2 [M + H]+ 1H NMR (DMSO-d6, 400 MHz): δ (ppm) 12.21 (s, 1H), 10.61 (s, 1H), 9.44 (s, 1H), 9.25 (s, 1H), 9.10 (s, 2H), 7.02 (s, 1H), 2.44 (s, 3H), 2.14 (s, 3H), 1.80 (s, 3H), 1.75 (s, 3H) |
| 724 | N-(4-((2-(1,1-difluoroethyl)pyrimidin-4-yl)amino)pyridin-2-yl)acetamide 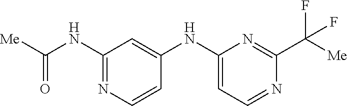 [SMILES: CC(=O)Nc1cc(Nc2ccnc(C(C)(F)F)n2)ccn1] LCMS m/z = 294.1 [M + H]⁺; ¹H NMR (400 MHz, DMSO-d₆) δ ppm: 10.38 (br s, 2H), 8.51 (br s, 1H), 8.33 (br s, 1H), 8.19 (br s, 1H), 7.74 (br s, 1H), 7.00 (br s, 1H), 2.01-2.11 (m, 6H). |
| 725 | N-(4-((2-(1,1-difluoroethyl)-6-methylpyrimidin-4-yl)amino)-5-(dimethylamino)pyridin-2- yl)acetamide 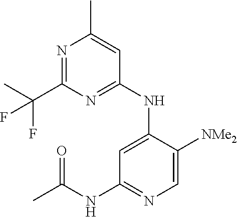 [SMILES: CC(=O)Nc1cc(Nc2cc(C)nc(C(C)(F)F)n2)c(N(C)C)cn1] LCMS m/z = 351.1 [M + H]⁺; ¹H NMR (500 MHz, DMSO-d₆) δ ppm: 10.26 (s, 1H), 9.13 (s, 1H), 8.99 (s, 1H), 8.08 (s, 1H), 7.22 (s, 1H), 2.67 (s, 6H), 2.40 (s, 3H), 2.14-2.03 (m, 6H). |
| 726 | N-(4-((2-(1,1-difluoroethyl)-6-methylpyrimidin-4-yl)amino)-5-(pyrrolidin-1-yl)pyridin-2- yl)acetamide 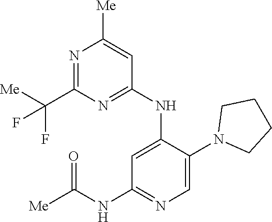 [SMILES: CC(=O)Nc1cc(Nc2cc(C)nc(C(C)(F)F)n2)c(N2CCCC2)cn1] LCMS m/z = 377.1 [M + H]⁺; ¹H NMR (400 MHz, CDCl₃) δ ppm: 8.86 (s, 1H), 8.04 (s, 1H), 7.95 (s, 2H), 6.85 (s, 1H), 3.12-3.08 (m, 4H), 2.56 (s, 3H), 2.23-2.11 (m, 6H), 2.03-2.00 (m, 4H). |
| 727 | N-(4-((2-(1,1-difluoroethyl)-6-methylpyrimidin-4-yl)amino)-5-morpholinopyridin-2- yl)acetamide 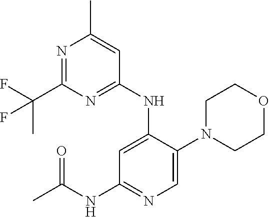 [SMILES: CC(=O)Nc1cc(Nc2cc(C)nc(C(C)(F)F)n2)c(N2CCOCC2)cn1] LCMS m/z = 393.1 [M + H]⁺; ¹H NMR (400 MHz, CDCl₃) δ ppm: 9.00 (s, 1H), 8.22 (s, 1H), 8.03 (s, 1H), 7.98 (br s, 1H), 6.82 (s, 1H), 3.91-3.89 (m, 4H), 2.96-2.93 (m, 4H), 2.56 (s, 3H), 2.21-2.11 (m, 6H). |
| 728 | N-(4-((2-(1,1-difluoroethyl)pyrimidin-4-yl)amino)-5-(4-methyl-1H-pyrazol-1-yl)pyridin-2- yl)acetamide 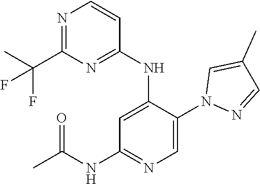 [SMILES: CC(=O)Nc1cc(Nc2ccnc(C(C)(F)F)n2)c(-n2cc(C)cn2)cn1] LCMS m/z = 374.1 [M + H]⁺; ¹H NMR (400 MHz, DMSO-d₆) δ ppm: 10.61 (s, 1H), 10.14 (s, 1H), 8.99 (s, 1H), 8.51 (d, J = 5.6 Hz, 1H), 8.37 (s, 1H), 8.03 (s, 1H), 7.65 (s, 1H), 7.07 (d, J = 5.6 Hz, 1H), 2.12-2.09 (m, 6H), 2.02 (t, J = 19.2 Hz, 3H). |
| 729 | N-(4-((2-(1,1-difluoroethyl)-6-methylpyrimidin-4-yl)amino)-5-(4-methyl-1H-pyrazol-1- yl)pyridin-2-yl)acetamide 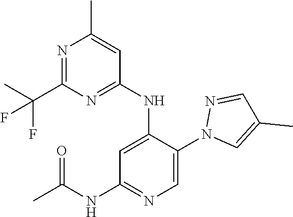 [SMILES: CC(=O)Nc1cc(Nc2cc(C)nc(C(C)(F)F)n2)c(-n2cc(C)cn2)cn1] LCMS m/z = 388 [M + H]⁺; ¹H NMR (400 MHz, DMSO-d₆) δ ppm: 10.57 (s, 1H), 10.09 (s, 1H), 9.02 (s, 1H), 8.36 (s, 1H), 8.05 (s, 1H), 7.67 (s, 1H), 6.93 (s, 1H), 2.39 (s, 3H), 2.11 (s, 3H), 2.10 (s, 3H), 2.03 (t, J = 18.8 Hz, 3H). |
| 730 | N-(4-((2-(1,1-difluoroethyl)pyrimidin-4-yl)amino)-5-(4-methoxy-1H-pyrazol-1-yl)pyridin-2- yl)acetamide 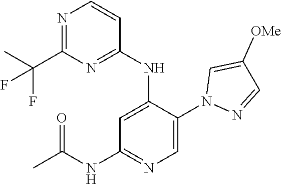 [SMILES: COc1cnn(-c2cnc(NC(C)=O)cc2Nc2ccnc(C(C)(F)F)n2)c1] LCMS m/z = 390.1 [M + H]⁺; ¹H NMR (400 MHz, MeOH-d₄) δ ppm: 9.17 (s, 1H), 8.46 (d, J = 6.0 Hz, 1H), 8.33 (s, 1H), 7.84 (s, 1H), 7.61 (s, 1H), 7.02 (d, J = 6.0 Hz, 1H), 3.82 (s, 3H), 2.20 (s, 3H), 2.07 (t, J = 19.2 Hz, 3H). |
| 731 | N-(4-((2-(1,1-difluoroethyl)-6-methylpyrimidin-4-yl)amino)-5-(4-methoxy-1H-pyrazol-1- yl)pyridin-2-yl)acetamide 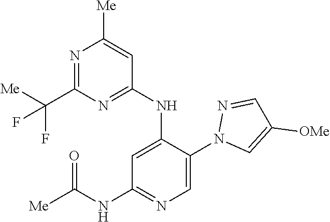 [SMILES: COc1cnn(-c2cnc(NC(C)=O)cc2Nc2cc(C)nc(C(C)(F)F)n2)c1] LCMS m/z = 404.3 [M + H]⁺; ¹H NMR (400 MHz, MeOH-d₄) δ ppm: 9.17 (s, 1H), 8.33 (s, 1H), 7.85 (s, 1H), 7.63 (s, 1H), 6.90 (s, 1H), 3.84 (s, 3H), 2.48 (s, 3H), 2.22 (s, 3H), 2.08 (t, J = 18.8 Hz, 3H). |
| 732 | N-(5-(4-cyanO1H-pyrazol-1-yl)-4-((2-(1,1-difluoroethyl)pyrimidin-4-yl)amino)pyridin-2- yl)acetamide 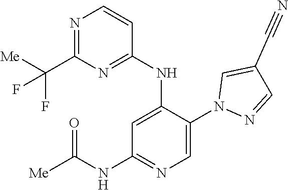 [SMILES: CC(=O)Nc1cc(Nc2ccnc(C(C)(F)F)n2)c(-n2cc(C#N)cn2)cn1] LCMS m/z = 385.1 [M + H]⁺; ¹H NMR (500 MHz, CDCl₃) δ ppm: 9.42 (s, 1H), 9.29 (s, 1H), 8.59 (d, J = 5.5 Hz, 1H), 8.25-8.22 (m, 2H), 8.18 (s, 1H), 7.99 (s, 1H), 6.91 (d, J = 5.5 Hz, 1H), 2.26 (s, 3H), 2.12 (t, J = 19.0 Hz, 3H). |
| 733 | N-(5-(4-cyanO1H-pyrazol-1-yl)-4-((2-(1,1-difluoroethyl)-6-methylpyrimidin-4- yl)amino)pyridin-2-yl)acetamide 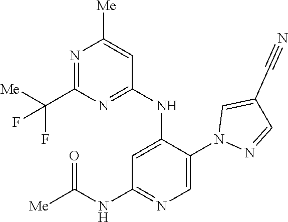 [SMILES: CC(=O)Nc1cc(Nc2cc(C)nc(C(C)(F)F)n2)c(-n2cc(C#N)cn2)cn1] LCMS m/z = 399.1 [M + H]⁺; ¹H NMR (400 MHz, DMSO-d₆) δ ppm: 10.70 (s, 1H), 9.57 (br s, 1H), 8.99 (s, 1H), 8.88 (s, 1H), 8.35 (s, 1H), 8.30 (s, 1H), 6.85 (s, 1H), 2.38 (s, 3H), 2.12 (s, 3H), 2.98 (t, J = 19.2 Hz, 3H). |
| 734 | N-(4-((2-(1,1-difluoroethyl)pyrimidin-4-yl)amino)-5-(3-methyl-1H-1,2,4-triazol-1- yl)pyridin-2-yl)acetamide 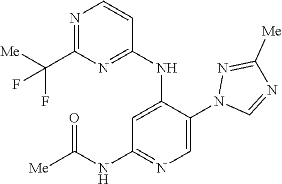 [SMILES: CC(=O)Nc1cc(Nc2ccnc(C(C)(F)F)n2)c(-n2cnc(C)n2)cn1] LCMS m/z = 375.2 [M + H]⁺; ¹H NMR (400 MHz, DMSO-d₆) δ ppm: 10.68 (s, 1H), 9.64 (br s, 1H), 8.96 (s, 1H), 8.76 (s, 1H), 8.51 (d, J = 5.6 Hz, 1H), 8.33 (s, 1H), 7.06 (d, J = 5.6 Hz, 1H), 2.31 (s, 3H), 2.12 (s, 3H), 2.01 (t, J = 18.8 Hz, 3H) |
| 735 | N-(4-((2-(1,1-difluoroethyl)-6-methylpyrimidin-4-yl)amino)-5-(3-methyl-1H-1,2,4-triazol-1- yl)pyridin-2-yl)acetamide 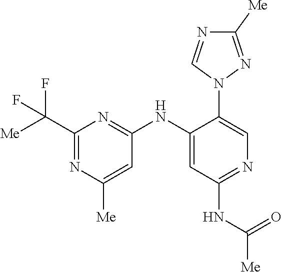 [SMILES: CC(=O)Nc1cc(Nc2cc(C)nc(C(C)(F)F)n2)c(-n2cnc(C)n2)cn1] LCMS m/z = 389.1 [M + H]⁺; ¹H NMR (400 MHz, DMSO-d₆) δ ppm: 10.66 (s, 1H), 9.52 (br s, 1H), 8.99 (s, 1H), 8.75 (s, 1H), 8.31 (s, 1H), 6.91 (s, 1H), 2.38 (s, 3H), 2.33 (s, 3H), 2.12 (s, 3H), 2.01 (t, J = 19.2 Hz, 3H). |
| 736 | N-(5-(5-(cyanomethyl)-4,5,6,7-tetrahydropyrazolo[1,5-a]pyrazin-2-yl)-4-((2-(1,1- difluoroethyl)pyrimidin-4-yl)amino)pyridin-2-yl)acetamide 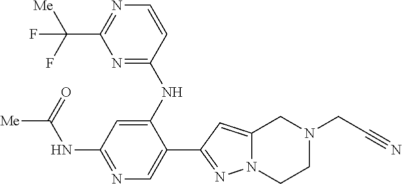 [SMILES: CC(=O)Nc1cc(Nc2ccnc(C(C)(F)F)n2)c(-c2cc3n(n2)CCN(CC#N)C3)cn1] LCMS m/z = 454.3 [M + H]⁺; ¹H NMR (400 MHz, CDCl₃) δ ppm: 11.40 (s, 1H), 9.36 (s, 1H), 8.54 (d, J = 6.0 Hz, 1H), 8.45 (s, 1H), 8.12 (s, 1H), 6.92 (d, J = 6.0 Hz, 1H), 6.44 (s, 1H), 4.37 (t, J = 5.6 Hz, 2H), 3.95 (s, 2H), 3.80 (s, 2H), 3.17 (t, J = 5.6 Hz, 2H), 2.25-2.14 (m, 6H). |
| 737 | N-(4-((2-(1,1-difluoroethyl)-6-methylpyrimidin-4-yl)amino)-5-(5- ((dimethylamino)methyl)thiazol-2-yl)pyridin-2-yl)acetamide 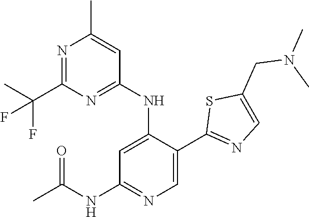 [SMILES: CC(=O)Nc1cc(Nc2cc(C)nc(C(C)(F)F)n2)c(-c2ncc(CN(C)C)s2)cn1] LCMS m/z = 448.2 [M + H]⁺; ¹H NMR (400 MHz, CDCl₃) δ ppm: 12.31 (s, 1H), 9.51 (s, 1H), 8.59 (s, 1H), 7.92 (s, 1H), 7.72 (s, 1H), 6.78 (s, 1H), 3.80 (br s, 2H), 2.56 (s, 3H), 2.39 (s, 6H), 2.25-2.16 (m, 6H). |
| 738 | N-(4-((2-(1,1-difluoroethyl)-6-methylpyrimidin-4-yl)amino)-5-(5-(pyrrolidin-1- ylmethyl)thiazol-2-yl)pyridin-2-yl)acetamide 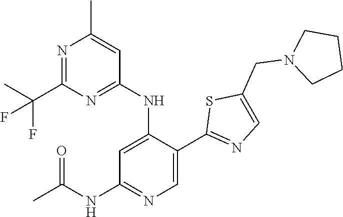 [SMILES: CC(=O)Nc1cc(Nc2cc(C)nc(C(C)(F)F)n2)c(-c2ncc(CN3CCCC3)s2)cn1] LCMS m/z = 474.2 [M + H]⁺; ¹H NMR (500 MHz, CDCl₃) δ ppm: 12.36 (s, 1H), 9.51 (s, 1H), 8.59 (s, 1H), 7.89 (s, 1H), 7.68 (s, 1H), 6.77 (s, 1H), 3.89 (s, 2H), 2.65-2.58 (m, 4H), 2.55 (s, 3H), 2.24-2.16 (m, 6H), 1.84 (s, 4H). |
| 739 | N-(4-((2-(1,1-difluoroethyl)-6-methylpyrimidin-4-yl)amino)-5-(1-methyl-5- (morpholinomethyl)-1H-pyrazol-3-yl)pyridin-2-yl)acetamide 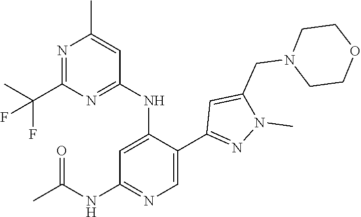 [SMILES: CC(=O)Nc1cc(Nc2cc(C)nc(C(C)(F)F)n2)c(-c2cc(CN3CCOCC3)n(C)n2)cn1] LCMS m/z = 487.3 [M + H]⁺; ¹H NMR (400 MHz, CDCl₃) δ ppm: 11.40 (s, 1 H), 9.35 (s, 1 H), 8.43 (s, 1 H), 8.08 (s, 1H), 6.75 (s, 1H), 6.53 (s, 1H), 4.03 (s, 3H), 3.72 (s, 4H), 3.57 (s, 2H), 2.55 (s, 3H), 2.49 (s, 4H), 2.23-2.14 (m, 6H). |
| 740 | N-(4′-((2-(1,1-difluoroethyl)-6-methylpyrimidin-4-yl)amino)-4-((dimethylamino)methyl)- [2,3′-bipyridin]-6′-yl)acetamide 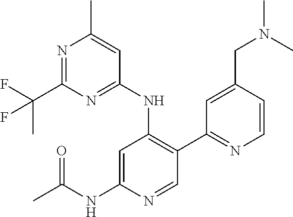 [SMILES: CC(=O)Nc1cc(Nc2cc(C)nc(C(C)(F)F)n2)c(-c2cc(CN(C)C)ccn2)cn1] LCMS m/z = 442.2 [M + H]⁺; ¹H NMR (400 MHz, CDCl₃) δ ppm: 12.71 (s, 1H), 9.31 (s, 1H), 8.64-8.61 (m, 2H), 8.02 ( s, 1H), 7.82 (s, 1H), 7.33 (d, J = 4.8 Hz, 1H), 6.76 (s, 1H), 3.60 (s, 2H), 2.53 (s, 3H), 2.35 (s, 6H), 2.24-2.13 (m, 6H). |
| 741 | N-(4-((2-(1,1-difluoroethyl)-6-methylpyrimidin-4-yl)amino)-5-(5-(1- (dimethylamino)ethyl)pyrazin-2-yl)pyridin-2-yl)acetamide 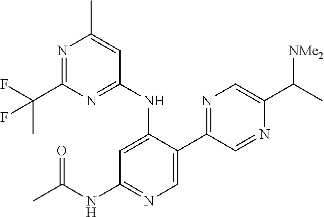 [SMILES: CC(=O)Nc1cc(Nc2cc(C)nc(C(C)(F)F)n2)c(-c2cnc(C(C)N(C)C)cn2)cn1] LCMS m/z = 457.2 [M + H]⁺; ¹H NMR (400 MHz, CDCl₃) δ ppm: 11.94 (s, 1H), 9.33 (s, 1H), 9.07 (s, 1H), 8.75 (s, 1H), 8.68 (s, 1H), 7.94 (s, 1H), 6.83 (s, 1H), 3.69-3.76 (m, 1H), 2.57 (s, 3H), 2.36 (s, 6H), 2.28 (s, 3H), 2.19 (t, J = 18.8 Hz, 3H), 1.51 (d, J = 6.8 Hz, 3H). |
| 742 | N-(4-((2-(1,1-difluoroethyl)-6-methylpyrimidin-4-yl)amino)-5-(5-(1-(pyrrolidin-1- yl)ethyl)pyrazin-2-yl)pyridin-2-yl)acetamide 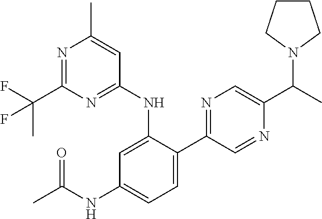 [SMILES: CC(=O)Nc1ccc(-c2cnc(C(C)N3CCCC3)cn2)c(Nc2cc(C)nc(C(C)(F)F)n2)c1] LCMS m/z = 483.2 [M + H]⁺; ¹H NMR (500 MHz, CDCl₃) δ ppm: 11.94 (s, 1H), 9.29 (s, 1H), 9.03 (s, 1H), 8.71 (s, 1H), 8.65 (s, 1H), 8.01 (s, 1H), 6.80 (s, 1H), 3.57 (q, J = 6.5 Hz, 1H), 2.65-2.68 (m, 2H), 2.66 (s, 3H), 2.56-2.53 (m, 2H), 2.49 (s, 3H), 2.22 (t, J = 18.5 Hz, 3H), 1.84-1.82 (m, 4H), 1.51 (d, J = 6.5 Hz, 3H). |
| 743 | N-(4′-((2-(1,1-difluoroethyl)-6-methylpyrimidin-4-yl)amino)-4-(1-(dimethylamino)ethyl)- [2,3′-bipyridin]-6′-yl)acetamide 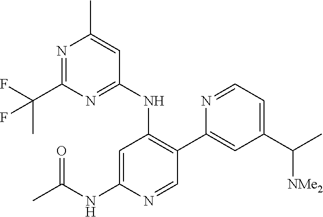 [SMILES: CC(=O)Nc1cc(Nc2cc(C)nc(C(C)(F)F)n2)c(-c2cc(C(C)N(C)C)ccn2)cn1] LCMS m/z = 456.2 [M + H]⁺; ¹H NMR (500 MHz, DMSO-d₆) δ ppm: 12.70 (s, 1H), 10.56 (s, 1H), 9.20 (s, 1H), 8.77 (s, 1H), 8.70 (d, J = 5.2 Hz, 1H), 7.92 (s, 1H), 7.38 (d, J = 5.2 Hz, 1H), 7.04 (s, 1H), 3.40-3.43 (m, 1H), 2.41 (s, 3H), 2.14-2.01 (m, 12H), 1.30 (d, J = 5.2 Hz, 3H). |
| 744 | N-(4-((2-(1,1-difluoroethyl)-6-methylpyrimidin-4-yl)amino)-5-(1,3,4-thiadiazol-2- yl)pyridin-2-yl)acetamide 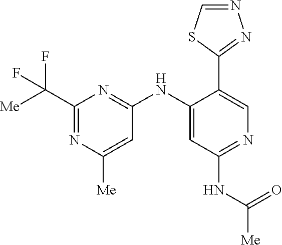 LCMS m/z = 392.1 [M + H]⁺; ¹H NMR (400 MHz, CDCl₃) δ ppm: 12.18 (s, 1H), 9.89 (s, 1H), 9.39 (br s, 1H), 9.18 (s, 1H), 8.54 (s, 1H), 6.16 (s, 1H), 2.60 (s, 3H), 2.32 (s, 3H), 2.21 (t, J = 18.8, Hz, 3H). |
| 745 | N-(4-((2-(1,1-difluoroethyl)-6-methylpyrimidin-4-yl)amino)-5-(6-(2- methoxyethyl)pyrimidin-4-yl)pyridin-2-yl)acetamide 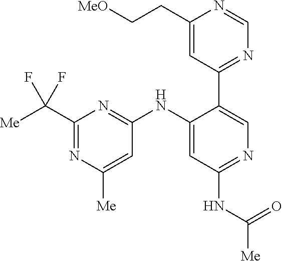 [SMILES: COCCc1cc(-c2cnc(NC(C)=O)cc2Nc2cc(C)nc(C(C)(F)F)n2)ncn1] LCMS m/z = 444.3 [M + H]⁺; ¹H NMR (500 MHz, CDCl₃) δ ppm: 12.59 (s, 1H), 9.39 (s, 1H), 9.21 (s, 1H), 8.66 (s, 1H), 7.94 (s, 1H), 7.69 (s, 1H), 6.78 (s, 1H), 3.84 (t, J = 6.5 Hz, 2H), 3.37 (s, 3H), 3.10 (t, J = 6.0 Hz, 2H), 2.56 (s, 3H), 2.25 (s, 3H), 2.18 (t, J = 19.0 Hz, 3H). |
| 746 | N-(4-((2-(2-oxabicyclo[2.1.1]hexan-4-yl)-6-methylpyrimidin-4-yl)amino)-5-(6- methylpyrimidin-4-yl)pyridin-2-yl)acetamide 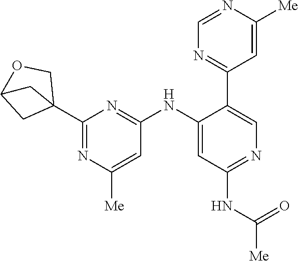 [SMILES: CC(=O)Nc1cc(Nc2cc(C)nc(C34COC(C3)C4)n2)c(-c2cc(C)ncn2)cn1] LCMS m/z = 418.2 [M + H]⁺; ¹H NMR (500 MHz, CDCl₃) δ ppm: 12.87 (s, 1H), 9.71 (s, 1H), 9.30 (s, 1H), 9.20-9.18 (m, 1H), 8.61 (s, 1H), 7.63 (s, 1H), 6.64 (s, 1H), 4.68 (s, 1H), 4.17 (s, 2H), 2.67 (s, 3H), 2.53 (s, 3H), 2.52-2.51 (m, 2H), 2.29 (s, 3H), 2.06-2.05 (m, 2H). |
| 747 | N-(4-((2-(2-oxabicyclo[2.1.1]hexan-4-yl)-6-methylpyrimidin-4-yl)amino)-5-(2- methylpyrimidin-4-yl)pyridin-2-yl)acetamide 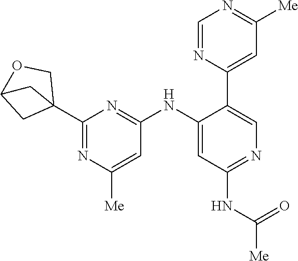 [SMILES: CC(=O)Nc1cc(Nc2cc(C)nc(C34COC(C3)C4)n2)c(-c2cc(C)ncn2)cn1] LCMS m/z = 418.2 [M + H]⁺; ¹H NMR (500 MHz, CDCl₃) δ ppm: 12.94 (s, 1H), 9.51 (s, 1H), 8.74 (d, J = 5.6 Hz, 1H), 8.65 (s, 1H), 8.37 (s, 1H), 7.57 (d, J = 5.6 Hz, 1H), 6.58 (s, 1H), 4.67 (s, 1H), 4.16 (s, 2H), 2.89 (s, 3H), 2.49 (s, 3H), 2.47 (d, J = 4.8 Hz, 2H), 2.26 (s, 3H), 2.03- 2.05 (m, 2H). |
| 748 | N-(4-((2-(2-oxabicyclo[2.1.1]hexan-4-yl)-6-methylpyrimidin-4-yl)amino)-5-(pyrimidin-4- yl)pyridin-2-yl)acetamide 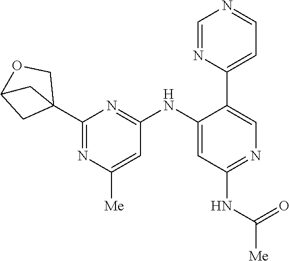 [SMILES: CC(=O)Nc1cc(Nc2cc(C)nc(C34COC(C3)C4)n2)c(-c2ccncn2)cn1] LCMS m/z = 404.2 [M + H]⁺; ¹H NMR (400 MHz, MeOH-d₄) δ ppm: 9.51 (s, 1H), 9.28 (s, 1H), 8.82-8.79 (m, 2H), 8.05 (d, J = 6.0 Hz, 1H), 6.81 (s, 1H), 4.64 (s, 1H), 4.11 (s, 2H), 2.46- 2.42 (m, 5H), 2.21 (s, 3H), 1.99-1.97 (m, 2H). |
| 749 | N-(4-((2-(2-oxabicyclo[2.1.1]hexan-4-yl)-6-methylpyrimidin-4-yl)amino)-5-(6- methoxypyridazin-3-yl)pyridin-2-yl)acetamide 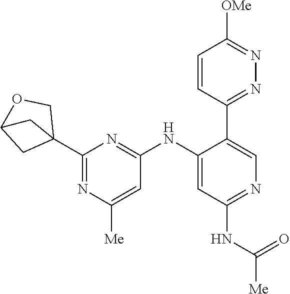 [SMILES: COc1ccc(-c2cnc(NC(C)=O)cc2Nc2cc(C)nc(C34COC(C3)C4)n2)nn1] LCMS m/z = 434.3 [M + H]⁺; ¹H NMR (500 MHz, CDCl₃) δ ppm: 12.16 (s, 1H), 9.62 (s, 1H), 8.44 (s, 1H), 8.16 (s, 1H), 7.86 (d, J = 9.5 Hz, 1H), 7.16 (d, J = 9.0 Hz, 1H), 6.66 (s, 1H), 4.66 (s, 1H), 4.22 (s, 3H), 4.16 (s, 2H), 2.48-2.45 (m, 5H), 2.24 (s, 3H), 2.05-2.03 (m, 2H). |
| 750 | N-(4-((2-(2-oxabicyclo[2.1.1]hexan-4-yl)-6-methylpyrimidin-4-yl)amino)-5-(6- methoxypyrimidin-4-yl)pyridin-2-yl)acetamide 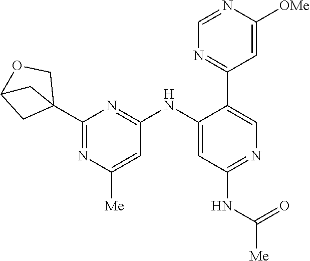 [SMILES: COc1cc(-c2cnc(NC(C)=O)cc2Nc2cc(C)nc(C34COC(C3)C4)n2)ncn1] LCMS m/z = 434.2 [M + H]⁺; ¹H NMR (400 MHz, CDCl₃) δ ppm: 12.35 (s, 1H), 9.38 (s, 1H), 8.63 (s, 1H), 8.59 (d, J = 5.6 Hz, 1H), 7.99 (s, 1H), 7.37 (d, J = 5.6 Hz, 1H), 6.96 (s, 1H), 4.66 (s, 1H), 4.17 (s, 3H), 4.13 (s, 2H), 2.48 (s, 3H), 2.43-2.41 (m, 2H), 2.25 (s, 3H), 2.03-2.01 (m, 2H). |
| 751 | N-(4′-((2-(2-oxabicyclo[2.1.1]hexan-4-yl)-6-methylpyrimidin-4-yl)amino)-5-cyanO[2,3′- bipyridin]-6′-yl)acetamide 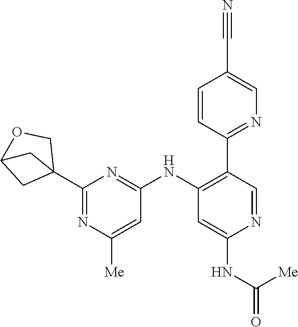 [SMILES: CC(=O)Nc1cc(Nc2cc(C)nc(C34COC(C3)C4)n2)c(-c2ccc(C#N)cn2)cn1] LCMS m/z = 428.3 [M + H]⁺; ¹H NMR (500 MHz, CDCl₃) δ ppm: 12.05 (s, 1H), 9.53 (s, 1H), 8.98 (d, J = 2.0 Hz, 1H), 8.58 (s, 1H), 8.18 (s, 1H), 8.10-8.08 (m, 1H), 7.92 (d, J = 8.5 Hz, 1H), 6.59 (s, 1H), 4.66 (s, 1H), 4.15 (s, 2H), 2.48-2.45 (m, 5H), 2.26 (s, 3H), 2.04-2.03 (m, 2H). |
| 752 | N-(4′-((2-(2-oxabicyclo[2.1.1]hexan-4-yl)-6-methylpyrimidin-4-yl)amino)-5-fluorO[2,3′- bipyridin]-6′-yl)acetamide 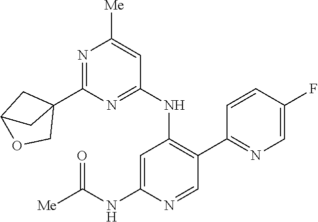 [SMILES: CC(=O)Nc1cc(Nc2cc(C)nc(C34COC(C3)C4)n2)c(-c2ccc(F)cn2)cn1] LCMS m/z = 421.2 [M + H]⁺; ¹H NMR (400 MHz, CDCl₃) δ ppm: 11.84 (s, 1H), 9.43 (s, 1H), 8.57 (d, J = 2.8 Hz, 1H), 8.48 (s, 1H), 7.90 (s, 1H), 7.79-7.76 (m, 1H), 7.65-7.55 (m, 1H), 6.55 (s, 1H), 4.66 (s, 1H), 4.15 (s, 2H), 2.45-2.43 (m, 5H), 2.23 (s, 3H), 2.05-2.01 (m, 2H). |
| 753 | N-(4-((2-(1,1-difluoroethyl)-6-methylpyrimidin-4-yl)amino)-5-(5-methoxypyridazin-3- yl)pyridin-2-yl)acetamide 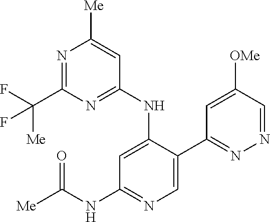 [SMILES: COc1cnnc(-c2cnc(NC(C)=O)cc2Nc2cc(C)nc(C(C)(F)F)n2)c1] LCMS m/z = 416.2 [M + H]⁺; ¹H NMR (400 MHz, MeOH-d₄) δ ppm: 9.33 (s, 1H), 8.92 (d, J = 2.4 Hz, 1H), 8.77 (s, 1H), 7.75 (d, J = 2.4 Hz, 1H), 7.03 (s, 1H), 4.09 (s, 3H), 2.51 (s, 3H), 2.24 (s, 3H), 2.12 (t, J = 18.8 Hz, 3H). |
| 754 | N-(4-((2-(1,1-difluoroethyl)-6-methylpyrimidin-4-yl)amino)-5-(5-(2-methoxyethyl)-1,3,4- thiadiazol-2-yl)pyridin-2-yl)acetamide 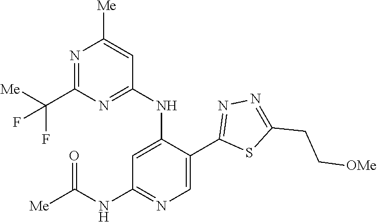 [SMILES: COCCc1nnc(-c2cnc(NC(C)=O)cc2Nc2cc(C)nc(C(C)(F)F)n2)s1] LCMS m/z = 450.1 [M + H]⁺; ¹H NMR (400 MHz, DMSO-d₆) δ ppm: 11.38 (br s, 1H), 10.72 (s, 1H), 9.24 (s, 1H), 8.69 (s, 1H), 7.04 (s, 1H), 3.71 (t, J = 5.8 Hz, 2H), 3.40-3.38 (m, 2H), 3.30 (s, 3H), 2.46 (s, 3H), 2.14-2.02 (m, 6H). |
| 755 | N-(4-((2-(1,1-difluoroethyl)-6-methylpyrimidin-4-yl)amino)-5-(5-(methoxymethyl)-1,3,4- thiadiazol-2-yl)pyridin-2-yl)acetamide 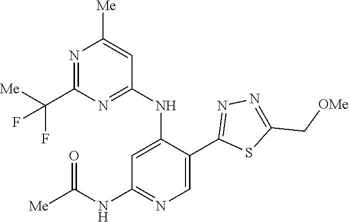 [SMILES: COCc1nnc(-c2cnc(NC(C)=O)cc2Nc2cc(C)nc(C(C)(F)F)n2)s1] LCMS m/z = 435.1 [M + H]⁺; ¹H NMR (400 MHz, CDCl₃) δ ppm: 11.87 (s, 1H), 9.66 (s, 1H), 8.52 (s, 1H), 7.89 (s, 1H), 6.86 (s, 1H), 4.91 (s, 2H), 3.54 (s, 3H), 2.55 (s, 3H), 2.27-2.16 (m, 6H). |
| 756 | N-(4-((2-(1,1-difluoroethyl)-6-methylpyrimidin-4-yl)amino)-5-(5-(2-methoxyethyl)thiazol- 2-yl)pyridin-2-yl)acetamide 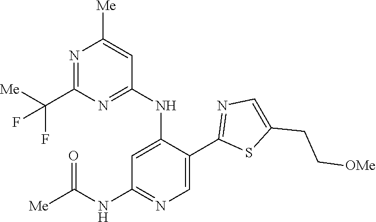 [SMILES: COCCc1cnc(-c2cnc(NC(C)=O)cc2Nc2cc(C)nc(C(C)(F)F)n2)s1] LCMS m/z = 449.2 [M + H]⁺; ¹H NMR (400 MHz, CDCl₃) δ ppm: 12.49 (br s, 1H), 9.60 (s, 1H), 8.59 (s, 1H), 8.51 (br s, 1H), 7.66 (s, 1H), 6.80 (s, 1H), 3.67 (d, J = 4.8 Hz, 2H), 3.44 (s, 3H), 3.16 (t, J = 4.8 Hz, 2H), 2.58 (s, 3H), 2.18-2.28 (m, 6H). |
| 757 | N-(4-((2-(1,1-difluoroethyl)-6-methylpyrimidin-4-yl)amino)-5-(5-(methoxymethyl)-1- methyl-1H-pyrazol-3-yl)pyridin-2-yl)acetamide 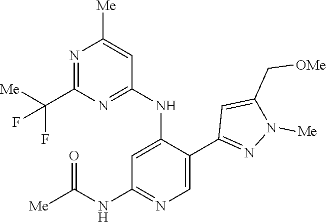 [SMILES: COCc1cc(-c2cnc(NC(C)=O)cc2Nc2cc(C)nc(C(C)(F)F)n2)nn1C] LCMS m/z = 432.2 [M + H]⁺; ¹H NMR (400 MHz, DMSO-d₆) δ ppm: 11.44 (s, 1H), 10.43 (s, 1H), 9.33 (s, 1H), 8.65 (s, 1H), 7.08 (s, 1H), 6.95 (s, 1H), 4.55 (s, 2H), 3.99 (s, 3H), 3.30 (s, 3H), 2.47 (s, 3H), 2.19-2.08 (m, 6H). |
| 758 | N-(4-((2-(1,1-difluoroethyl)-6-methylpyrimidin-4-yl)amino)-5-(5-(2-methoxypropan-2-yl)- 1-methyl-1H-pyrazol-3-yl)pyridin-2-yl)acetamide 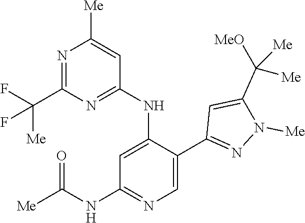 [SMILES: COC(C)(C)c1cc(-c2cnc(NC(C)=O)cc2Nc2cc(C)nc(C(C)(F)F)n2)nn1C] LCMS m/z = 460.2 [M + H]⁺; ¹H NMR (400 MHz, MeOH-d₄) δ ppm: 9.29 (s, 1H), 8.56 (s, 1H), 8.15 (br s, 1H), 6.94 (s, 1H), 6.73 (s, 1H), 4.14 (s, 3H), 3.13 (s, 3H), 2.51 (s, 3H), 2.20 (s, 3H), 2.12 (t, J = 18.8 Hz, 3H), 1.64 (s, 6H). |
| 759 | N-(4-((2-(1,1-difluoroethyl)-6-methylpyrimidin-4-yl)amino)-5-(7-methyl-5,6,7,8- tetrahydro[1,2,4]triazolo[1,5-a]pyrazin-2-yl)pyridin-2-yl)acetamide 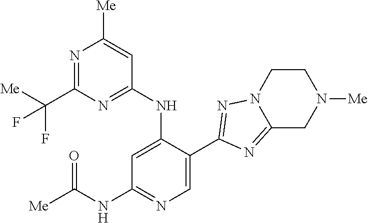 [SMILES: CC(=O)Nc1cc(Nc2cc(C)nc(C(C)(F)F)n2)c(-c2nc3n(n2)CCN(C)C3)cn1] LCMS m/z = 444.3 [M + H]⁺; ¹H NMR (400 MHz, DMSO-d₆) δ ppm: 11.33 (s, 1H), 10.54 (s, 1H), 9.36 (s, 1H), 8.88 (s, 1H), 7.03 (s, 1H), 4.31-4.24 (m, 2H), 3.81 (s, 2H), 2.98 (t, J = 6.0 Hz, 2H), 2.46 (d, J = 6.0 Hz, 6H), 2.18-2.08 (m, 6H). |
| 760 | N-(4-((2-(1,1-difluoroethyl)-6-methylpyrimidin-4-yl)amino)-5-(5,6-dihydrO8H- [1,2,4]triazolo[5,1-c][1,4]oxazin-2-yl)pyridin-2-yl)acetamide 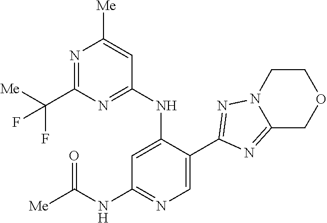 [SMILES: CC(=O)Nc1cc(Nc2cc(C)nc(C(C)(F)F)n2)c(-c2nc3n(n2)CCOC3)cn1] LCMS m/z = 431.1 [M + H]⁺; ¹H NMR (500 MHz, CDCl₃) δ ppm: 11.01 (s, 1H), 9.36 (s, 1H), 9.01 (s, 1H), 7.94 (s, 1H), 6.80 (s, 1H), 5.01 (s, 2H), 4.36 (t, J = 4.5 Hz, 2H), 4.23 (t, J = 4.5 Hz, 2H), 2.56 (s, 3H), 2.15-2.23 (m, 6H). |
| 761 | N-(4-((2-(1,1-difluoroethyl)-6-methylpyrimidin-4-yl)amino)-5-(isothiazol-3-yl)pyridin-2- yl)acetamide 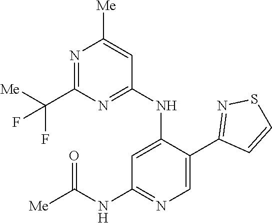 [SMILES: CC(=O)Nc1cc(Nc2cc(C)nc(C(C)(F)F)n2)c(-c2ccsn2)cn1] LCMS m/z = 391.1 [M + H]⁺; ¹H NMR (500 MHz, DMSO-d₆) δ ppm: 11.86 (s, 1H), 10.57 (s, 1H), 9.30 (s, 1H), 9.23 (d, J = 5.0 Hz, 1H), 8.91 (s, 1H), 8.11 (d, J = 5.0 Hz, 1H), 7.13 (s, 1H), 2.44 (s, 3H), 2.06-2.14 (m, 6H). |
| 762 | N-(4-((2-(1,1-difluoroethyl)-6-methylpyrimidin-4-yl)amino)-5-(3-(methoxymethyl)-1,2,4- thiadiazol-5-yl)pyridin-2-yl)acetamide 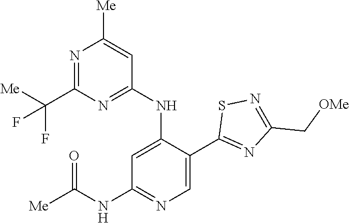 [SMILES: COCc1nsc(-c2cnc(NC(C)=O)cc2Nc2cc(C)nc(C(C)(F)F)n2)n1] LCMS m/z = 436.2 [M + H]⁺; ¹H NMR (500 MHz, CDCl₃) δ ppm: 11.90 (s, 1H), 9.57 (s, 1H), 8.62 (s, 1H), 8.04 (s, 1H), 6.76 (s, 1H), 4.84 (s, 2H), 3.60 (s, 3H), 2.58 (s, 3H), 2.26-2.16 (m, 6H). |
| 763 | N-(4-((2-(1,1-difluoroethyl)-6-methylpyrimidin-4-yl)amino)-5-(5-(dimethylamino)-1,2,4- thiadiazol-3-yl)pyridin-2-yl)acetamide 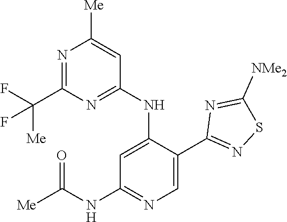 [SMILES: CC(=O)Nc1cc(Nc2cc(C)nc(C(C)(F)F)n2)c(-c2nsc(N(C)C)n2)cn1] LCMS m/z = 435.1 [M + H]⁺; ¹H NMR (400 MHz, CDCl₃) δ ppm: 12.40 (s, 1H), 10.51 (br s, 1H), 9.74 (s, 1H), 9.12 (s, 1H), 6.89 (s, 1H), 3.27 (s, 6H), 2.61 (s, 3H), 2.34 (s, 3H), 2.19 (t, J = 18.8 Hz, 3H). |
| 764 | N-(4-((2-(1,1-difluoroethyl)-6-methylpyrimidin-4-yl)amino)-5-(4-(2-methoxyethyl)-1H- pyrazol-1-yl)pyridin-2-yl)acetamide 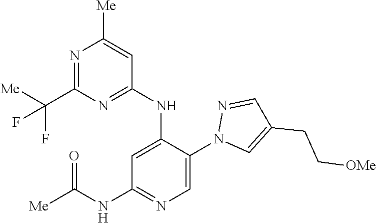 [SMILES: COCCc1cnn(-c2cnc(NC(C)=O)cc2Nc2cc(C)nc(C(C)(F)F)n2)c1] LCMS m/z = 432.0 [M + H]⁺; ¹H NMR (400 MHz, DMSO-d₆) δ ppm: 10.61 (s, 1H), 10.17 (s, 1H), 9.00 (s, 1H), 8.40 (s, 1H), 8.09 (s, 1H), 7.72 (s, 1H), 6.92 (s, 1H), 3.53-3.46 (m, 2H), 3.27 (s, 3H), 2.75-2.68 (m, 2H), 2.39 (s, 3H), 2.12 (s, 3H), 2.02 (t, J = 19.2 Hz, 3H). |
| 765 | N-(4-((2-(1,1-difluoroethyl)-6-methylpyrimidin-4-yl)amino)-5-(4-(methoxymethyl)-1H- pyrazol-1-yl)pyridin-2-yl)acetamide 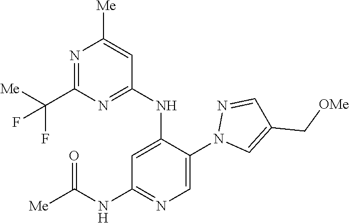 [SMILES: COCc1cnn(-c2cnc(NC(C)=O)cc2Nc2cc(C)nc(C(C)(F)F)n2)c1] LCMS m/z = 418.2 [M + H]⁺; ¹H NMR (400 MHz, MeOH-d₄) δ ppm: 9.16 (s, 1H), 8.34 (s, 1H), 8.08 (s, 1H), 7.85 (s, 1H), 6.87 (s, 1H), 4.46 (s, 2H), 3.38 (s, 3H), 2.46 (s, 3H), 2.20 (s, 3H), 2.06 (t, J = 18.8 Hz, 3H). |
| 766 | N-(5-(6-cyanopyrazin-2-yl)-4-((2-(1,1-difluoroethyl)pyrimidin-4-yl)amino)pyridin-2- yl)acetamide 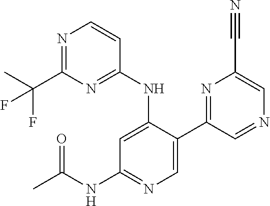 [SMILES: CC(=O)Nc1cc(Nc2ccnc(C(C)(F)F)n2)c(-c2cncc(C#N)n2)cn1] LCMS m/z = 397.0 [M + H]⁺; ¹H NMR (400 MHz, DMSO-d₆) δ ppm: 10.75 (s, 1H), 10.30 (br s, 1H), 9.22 (s, 1H), 9.11 (s, 1H), 8.71 (s, 1H), 8.61 (s, 1H), 8.49 (d, J = 5.6 Hz, 1H), 6.96 (d, J = 5.6 Hz, 1H), 2.14 (s, 3H), 1.89 (t, J = 18.8 Hz, 3H). |
| 767 | N-(4-((2-(1,1-difluoroethyl)pyrimidin-4-yl)amino)-5-(6-(difluoromethoxy)pyrimidin-4- yl)pyridin-2-yl)acetamide 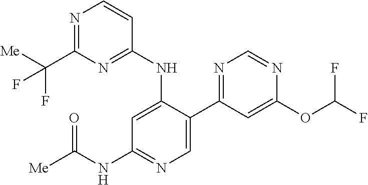 [SMILES: CC(=O)Nc1cc(Nc2ccnc(C(C)(F)F)n2)c(-c2cc(OC(F)F)ncn2)cn1] LCMS m/z = 438.0 [M + H]⁺; ¹H NMR (400 MHz, MeOH-d₄) δ ppm: 9.33 (s, 1H), 9.00 (s, 1H), 8.80 (s, 1H), 8.51 (d, J = 5.6 Hz, 1H), 7.73 (t, J = 71.6 Hz, 1H), 7.62 (s, 1H), 7.15 (d, J = 6.0 Hz, 1H), 2.21 (s, 3H), 2.11 (t, J = 18.8 Hz, 3H). |
| 768 | N-(4-((2-(1,1-difluoroethyl)-6-methylpyrimidin-4-yl)amino)-5-(5,6-dihydrO8H-imidazo[2,1- c][1,4]oxazin-2-yl)pyridin-2-yl)acetamide 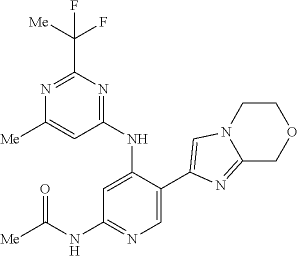 [SMILES: CC(=O)Nc1cc(Nc2cc(C)nc(C(C)(F)F)n2)c(-c2cn3c(n2)COCC3)cn1] LCMS m/z = 430.2 [M + H]⁺; ¹H NMR (500 MHz, CDCl₃) δ ppm: 11.90 ( s, 1H), 9.34 (s, 1H), 8.33 (s, 1H), 7.96 (s, 1H), 7.21 (s, 1H), 6.68 (s, 1H), 4.95 (s, 2H), 4.13 (s, 4H), 2.52 (s, 3H), 2.15-2.23 (m, 6H). |
| 769 | N-(4-((2-(1,1-difluoroethyl)-6-methylpyrimidin-4-yl)amino)-5-(6,7-dihydrO5H-pyrrolo[1,2- a]imidazol-2-yl)pyridin-2-yl)acetamide 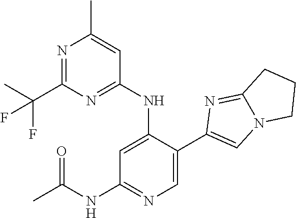 [SMILES: CC(=O)Nc1cc(Nc2cc(C)nc(C(C)(F)F)n2)c(-c2cn3c(n2)CCC3)cn1] LCMS m/z = 414.2 [M + H]⁺; ¹H NMR (400 MHz, CDCl₃) δ ppm: 12.68 (s, 1H), 9.62 (s, 1H), 9.21 (s, 1H), 6.78 (s, 1H), 4.13-4.07 (m, 2H), 3.04-2.99 (m, 2H), 2.73-2.69 (m, 2H), 2.56 (s, 3H), 2.55 (s, 3H), 2.19 (t, J = 18.8 Hz, 3H). |
| 770 | N-(4-((2-(1,1-difluoroethyl)-6-methylpyrimidin-4-yl)amino)-5-(2,3-dihydroimidazo[2,1- b]oxazol-6-yl)pyridin-2-yl)acetamide 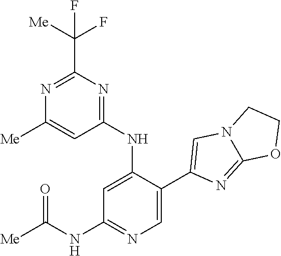 [SMILES: CC(=O)Nc1cc(Nc2cc(C)nc(C(C)(F)F)n2)c(-c2cn3c(n2)OCC3)cn1] LCMS m/z = 416.2 [M + H]⁺; ¹H NMR (400 MHz, CDCl₃) δ ppm: 11.56 (s, 1H), 9.42 (br s, 1H), 8.35 (br s, 1H), 8.24 (s, 1H), 6.95 (s, 1H), 6.73 (s, 1H), 5.11 (t, J = 7.6 Hz, 2H), 4.29 (t, J = 8.0 Hz, 2H), 2.52 (s, 3H), 2.24-2.14 (m, 6H). |
| 771 | N-(4-((2-(1,1-difluoroethyl)-6-methylpyrimidin-4-yl)amino)-5-(2-(4-methylpiperazin-1- yl)thiazol-4-yl)pyridin-2-yl)acetamide 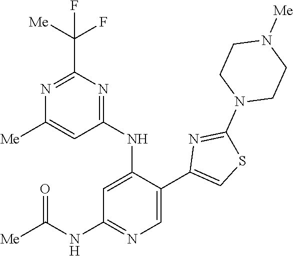 [SMILES: CC(=O)Nc1cc(Nc2cc(C)nc(C(C)(F)F)n2)c(-c2csc(N3CCN(C)CC3)n2)cn1] LCMS m/z = 489.2 [M + H]⁺; ¹H NMR (400 MHz, CDCl₃) δ ppm: 11.67 (s, 1H), 8.45 (s, 1H), 8.44 (s, 1H), 8.00 (s, 1H), 6.92 (s, 1H), 6.78 (s, 1H), 3.66 (t, J = 5.2 Hz, 4H), 2.60 (t, J = 5.2 Hz, 4H), 2.54 (s, 3H), 2.40 (s, 3H), 2.23 (s, 3H), 2.08 (t, J = 18.4 Hz, 3H). |
| 772 | N-(4-((2-(1,1-difluoroethyl)-6-methylpyrimidin-4-yl)amino)-5-(2-((2- methoxyethyl)(methyl)amino)thiazol-4-yl)pyridin-2-yl)acetamide 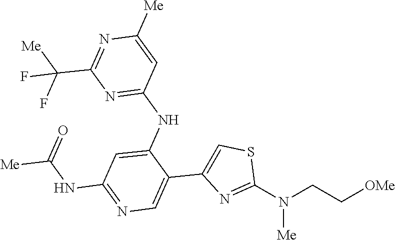 [SMILES: COCCN(C)c1nc(-c2cnc(NC(C)=O)cc2Nc2cc(C)nc(C(C)(F)F)n2)cs1] LCMS m/z = 478.2 [M + H]⁺; ¹H NMR (400 MHz, CDCl₃) δ ppm: 11.68 (s, 1H), 8.96 (s, 1H), 8.44 (s, 1H), 7.92 (s, 1H), 6.92 (s, 1H), 6.72 (s, 1H), 3.83 (t, J = 5.2 Hz, 2H), 3.73 (t, J = 5.2 Hz, 2H), 3.39 (s, 3H), 3.25 (s, 3H), 2.21 (s, 3H), 2.23-2.06 (m, 6H). |
| 773 | N-(4-((2-(1,1-difluoroethyl)pyrimidin-4-yl)amino)-5-(5-((2-methoxyethyl)(methyl)amino)- 1,3,4-thiadiazol-2-yl)pyridin-2-yl)acetamide 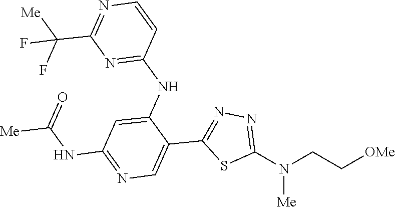 [SMILES: COCCN(C)c1nnc(-c2cnc(NC(C)=O)cc2Nc2ccnc(C(C)(F)F)n2)s1] LCMS m/z = 465.1 [M + H]⁺; ¹H NMR (400 MHz, DMSO-d₆) δ ppm: 11.49 (s, 1H), 10.66 (s, 1H), 9.27 (s, 1H), 8.61 (d, J = 5.6 Hz, 1H), 8.49 (s, 1H), 7.17 (d, J = 5.6 Hz, 1H), 3.70 (t, J = 5.2 Hz, 2H), 3.61 (t, J = 5.2 Hz, 2H), 3.28 (s, 3H), 3.19 (s, 3H), 2.16-2.06 (m, 6H). |
| 774 | N-(4-((2-(1,1-difluoroethyl)pyrimidin-4-yl)amino)-5-(5-(4-methylpiperazin-1-yl)-1,3,4- thiadiazol-2-yl)pyridin-2-yl)acetamide 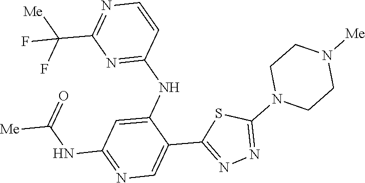 [SMILES: CC(=O)Nc1cc(Nc2ccnc(C(C)(F)F)n2)c(-c2nnc(N3CCN(C)CC3)s2)cn1] LCMS m/z = 476.2 [M + H]⁺; ¹H NMR (400 MHz, DMSO-d₆) δ ppm: 11.28 (s, 1H), 10.71 (s, 1H), 9.21 (s, 1H), 8.62 (d, J = 6.0 Hz, 1H), 8.54 (s, 1H), 7.15 (d, J = 5.6 Hz, 1H), 4.10-4.08 (m, 2H), 3.58-3.52 (m, 4H), 2.83 (s, 2H), 2.13-2.04 (m, 9H). |
| 775 | N-(4-((2-(1,1-difluoroethyl)pyrimidin-4-yl)amino)-5-(2-(difluoromethyl)pyrimidin-4- yl)pyridin-2-yl)acetamide 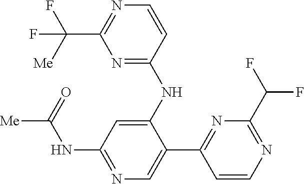 [SMILES: CC(=O)Nc1cc(Nc2ccnc(C(C)(F)F)n2)c(-c2ccnc(C(F)F)n2)cn1] LCMS m/z = 422.1 [M + H]⁺; ¹H NMR (500 MHz, CDCl₃) δ ppm: 13.10 ( s, 1H), 9.79 (s, 1H), 8.96 (d, J = 5.5 Hz, 1H), 8.90 (s, 1H), 8.82 (s, 1H), 8.64 (d, J = 6.0 Hz, 1H), 7.95 (d, J = 5.5 Hz, 1H), 6.98 (d, J = 5.5 Hz, 1H), 6.79 (t, J = 54.5 Hz, 1H), 2.31 (s, 3H), 2.21 (t, J = 19.0 Hz, 3H). |
| 776 | N-(4-((2-(1,1-difluoroethyl)-6-methylpyrimidin-4-yl)amino)-5-(2-(2- methoxyethoxy)pyrimidin-4-yl)pyridin-2-yl)acetamide 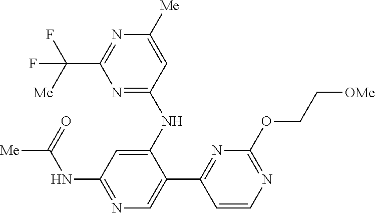 [SMILES: COCCOc1nccc(-c2cnc(NC(C)=O)cc2Nc2cc(C)nc(C(C)(F)F)n2)n1] LCMS m/z = 460.2 [M + H]⁺; ¹H NMR (400 MHz, CDCl₃) δ ppm: 11.50 (s, 1H), 10.68 (s, 1H), 8.99 (s, 1H), 8.78 (s, 1H), 8.55 (d, J = 7.2 Hz, 1H), 7.66 (d, J = 5.2 Hz, 1H), 6.89 (s, 1H), 4.51 (d, J = 4.4 Hz, 2H), 3.72 (d, J = 4.4 Hz, 2H), 3.33 (s, 3H), 2.42 (s, 3H), 2.13 (s, 3H), 2.02 (t, J = 19.2 Hz, 3H). |
| 777 | N-(4-((2-(1,1-difluoroethyl)-6-methylpyrimidin-4-yl)amino)-5-(4-(2- methoxyethoxy)pyrimidin-2-yl)pyridin-2-yl)acetamide 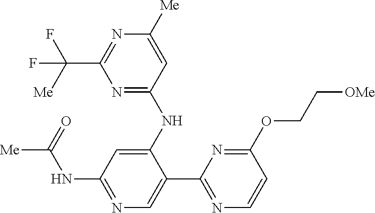 [SMILES: COCCOc1ccnc(-c2cnc(NC(C)=O)cc2Nc2cc(C)nc(C(C)(F)F)n2)n1] LCMS m/z = 460.2 [M + H]⁺; ¹H NMR (400 MHz, CDCl₃) δ ppm: 13.00 (s, 1H), 9.38 (s, 1H), 9.28 (s, 1H), 8.50 (d, J = 6.0 Hz, 1H), 8.10 (s, 1H), 6.86 (s, 1H), 6.74 (d, J = 6.0 Hz, 1H), 4.70 (t, J = 4.4 Hz, 2H), 3.83 (t, J = 4.4 Hz, 2H), 3.47 (s, 3H), 2.56 (s, 3H), 2.25-2.13 (m, 6H). |
| 778 | N-(4-((2-(1,1-difluoroethyl)pyrimidin-4-yl)amino)-5-(2-(2-morpholinoethyl)-2H-1,2,3- triazol-4-yl)pyridin-2-yl)acetamide 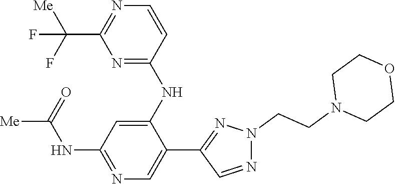 [SMILES: CC(=O)Nc1cc(Nc2ccnc(C(C)(F)F)n2)c(-c2cnn(CCN3CCOCC3)n2)cn1] LCMS m/z = 474.2 [M + H]⁺; ¹H NMR (500 MHz, DMSO-d₆) δ ppm: 10.58 (s, 1H), 10.34 (s, 1H), 9.07 (s, 1H), 8.72 (s, 1H), 8.58 (d, J = 6.0 Hz, 1H), 8.33 (s, 1H), 7.16 (d, J = 5.5 Hz, 1H), 4.66 (t, J = 6.5 Hz, 2H), 3.49 (t, J = 4.5 Hz, 4H), 2.87 (t, J = 6.5 Hz, 2H), 2.44 (s, 4H), 2.12-2.01 (m, 6H). |
| 779 | N-(4-((2-(1,1-difluoroethyl)pyrimidin-4-yl)amino)-5-(2-(2-(4-methylpiperazin-1-yl)ethyl)- 2H-1,2,3-triazol-4-yl)pyridin-2-yl)acetamide 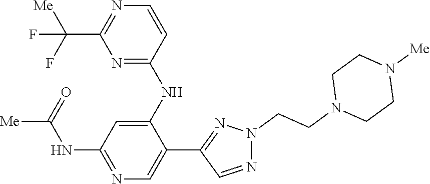 [SMILES: CC(=O)Nc1cc(Nc2ccnc(C(C)(F)F)n2)c(-c2cnn(CCN3CCN(C)CC3)n2)cn1] LCMS m/z = 487.3 [M + H]⁺; ¹H NMR (400 MHz, MeOH-d₄) δ ppm: 9.19 (s, 1H), 8.63 (s, 1H), 8.50 (d, J = 5.6 Hz, 1H), 8.16 (s, 1H), 7.13 (d, J = 6.0 Hz, 1H), 4.72 (t, J = 6.4 Hz, 2H), 3.05 (t, J = 6.4 Hz, 2H), 2.43-2.73 (m, 8H), 2.29 (s, 3H), 2.20 (s, 3H), 2.10 (t, J = 18.8 Hz, 3H) |
| 780 | N-(4-((2-(1,1-difluoroethyl)pyrimidin-4-yl)amino)-5-(5,6,7,8-tetrahydroimidazo[1,2- a]pyridin-2-yl)pyridin-2-yl)acetamide 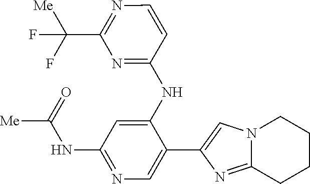 [SMILES: CC(=O)Nc1cc(Nc2ccnc(C(C)(F)F)n2)c(-c2cn3c(n2)CCCC3)cn1] LCMS m/z = 414.1 [M + H]⁺; ¹H NMR (400 MHz, DMSO-d₆) δ ppm: 13.05-12.52 (m, 1H), 10.37 (s, 1H), 9.23 (s, 1H), 8.54 (d, J = 5.6 Hz, 2H), 7.71 (s, 1H), 7.04 (d, J = 5.6 Hz, 1H), 4.03 (t, J = 5.6 Hz, 2H), 2.90 (t, J = 6.0 Hz, 2H), 2.08-2.18 (m, 6H), 1.88-1.98 (m, 4H). |
| 781 | N-(4-((2-(1,1-difluoroethyl)pyrimidin-4-yl)amino)-5-(5-(methoxymethyl)thiazol-2- yl)pyridin-2-yl)acetamide 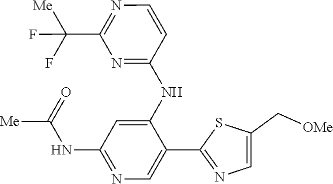 [SMILES: COCc1cnc(-c2cnc(NC(C)=O)cc2Nc2ccnc(C(C)(F)F)n2)s1] LCMS m/z = 421.2 [M + H]⁺; ¹H NMR (400 MHz, MeOH-d₄) δ ppm: 9.48 (s, 1H), 8.70 (s, 1H), 8.53 (J = 6.0 Hz, 1H), 7.87 (s, 1H), 7.12 (J = 6.0 Hz, 1H), 4.73 (s, 2H), 3.42 (s, 3H), 2.21 (s, 3H), 2.15 (J = 18.8 Hz, 3H). |
| 782 | N-(4-((2-(1,1-difluoroethyl)pyrimidin-4-yl)amino)-5-(5-((dimethylamino)methyl)thiazol-2- yl)pyridin-2-yl)acetamide 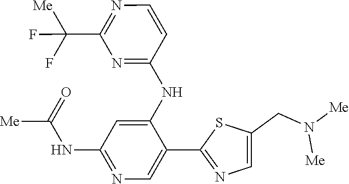 [SMILES: CC(=O)Nc1cc(Nc2ccnc(C(C)(F)F)n2)c(-c2ncc(CN(C)C)s2)cn1] LCMS m/z = 434.2 [M + H]⁺; ¹H NMR (400 MHz, CDCl₃) δ ppm: 12.47 (s, 1H), 9.52 (s, 1H), 8.61 (s, 1H), 8.56 (d, J = 5.6 Hz, 1H), 7.94 (s, 1H), 7.68 (s, 1H), 6.93 (d, J = 5.6 Hz, 1H), 3.74 (s, 2H), 2.35 (s, 6H), 2.25-2.16 (m, 6H). |
| 783 | N-(4-((2-(1,1-difluoroethyl)-6-methylpyrimidin-4-yl)amino)-5-(5-((4-methylpiperazin-1- yl)methyl)thiazol-2-yl)pyridin-2-yl)acetamide 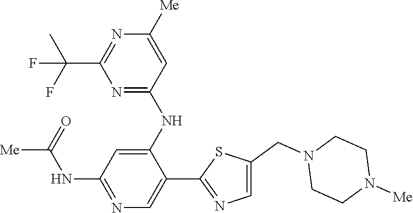 [SMILES: CC(=O)Nc1cc(Nc2cc(C)nc(C(C)(F)F)n2)c(-c2ncc(CN3CCN(C)CC3)s2)cn1] LCMS m/z = 503.3 [M + H]⁺; ¹H NMR (500 MHz, CDCl₃) δ ppm: 12.34 (s, 1H), 9.51 (s, 1H), 8.59 (s, 1H), 7.93 (s, 1H), 7.67 (s, 1H), 6.77 (s, 1H), 3.78 (s, 2H), 2.68-2.55 (m, 11H), 2.33 (s, 3H), 2.24-2.16 (m, 6H). |
| 784 | N-(4-((2-(1,1-difluoroethyl)-6-methylpyrimidin-4-yl)amino)-5-(4- ((dimethylamino)methyl)thiazol-2-yl)pyridin-2-yl)acetamide 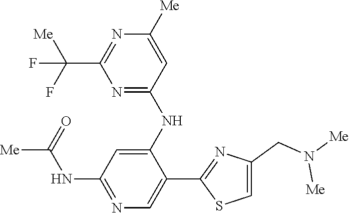 [SMILES: CC(=O)Nc1cc(Nc2cc(C)nc(C(C)(F)F)n2)c(-c2nc(CN(C)C)cs2)cn1] LCMS m/z = 448.1 [M + H]⁺; ¹H NMR (400 MHz, DMSO-d₆) δ ppm: 12.50 (s, 1H), 10.65 (s, 1H), 9.36 (s, 1H), 8.73 (s, 1H), 7.57 (s, 1H), 6.95 (s, 1H), 3.71 (s, 2H), 2.46 (s, 3H), 2.28 (s, 6H), 2.17-2.07 (m, 6H). |
| 785 | N-(4-((2-(1,1-difluoroethyl)-6-methylpyrimidin-4-yl)amino)-5-(4-((4-methylpiperazin-1- yl)methyl)thiazol-2-yl)pyridin-2-yl)acetamide 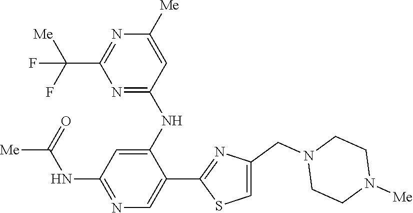 [SMILES: CC(=O)Nc1cc(Nc2cc(C)nc(C(C)(F)F)n2)c(-c2nc(CN3CCN(C)CC3)cs2)cn1] LCMS m/z = 503.3 [M + H]⁺; ¹H NMR (400 MHz, DMSO-d₆) δ ppm: 12.52 (s, 1H), 10.65 (s, 1H), 9.41 (s, 1H), 8.73 (s, 1H), 7.58 (s, 1H), 6.99 (s, 1H), 3.76 (s, 2H), 3.30 (s, 4H), 2.39- 2.29 (m, 4H), 2.20-2.06 (m, 12H). |
| 786 | N-(4-((2-(1,1-difluoroethyl)-6-methylpyrimidin-4-yl)amino)-5-(2- ((dimethylamino)methyl)thiazol-4-yl)pyridin-2-yl)acetamide 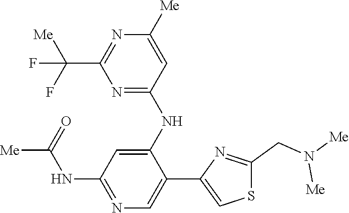 [SMILES: CC(=O)Nc1cc(Nc2cc(C)nc(C(C)(F)F)n2)c(-c2csc(CN(C)C)n2)cn1] LCMS m/z = 448.2 [M + H]⁺; ¹H NMR (500 MHz, CDCl₃) δ ppm: 11.43 (s, 1H), 9.23 (s, 1H), 8.48 (s, 1H), 8.19 (s, 1H), 7.54 (s, 1H), 6.76 (s, 1H), 3.95 (s, 2H), 2.54 (s, 3H), 2.46 (s, 6H), 2.24-2.16 (m, 6H). |
| 787 | N-(4-((2-(1,1-difluoroethyl)-6-methylpyrimidin-4-yl)amino)-5-(2-((4-methylpiperazin-1- yl)methyl)thiazol-4-yl)pyridin-2-yl)acetamide 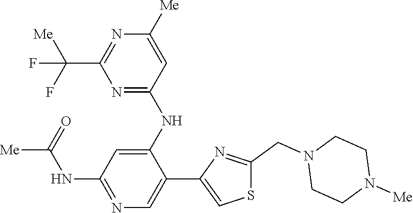 [SMILES: CC(=O)Nc1cc(Nc2cc(C)nc(C(C)(F)F)n2)c(-c2csc(CN3CCN(C)CC3)n2)cn1] LCMS m/z = 503.3 [M + H]⁺; ¹H NMR (500 MHz, CDCl₃) δ ppm: 11.39 (s, 1H), 9.23 (s, 1H), 8.48 (s, 1H), 7.90 (s, 1H), 7.52 (s, 1H), 6.75 (s, 1H), 4.01 (s, 2H), 2.74 (br s, 4H), 2.67-2.49 (m, 7H), 2.35 (s, 3H), 2.23-2.13 (m, 6H). |
| 788 | N-(5-(2-(azetidin-1-yl)thiazol-4-yl)-4-((2-(1,1-difluoroethyl)pyrimidin-4-yl)amino)pyridin- 2-yl)acetamide 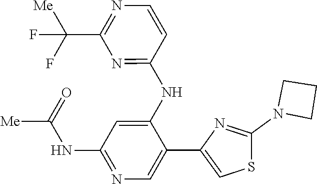 [SMILES: CC(=O)Nc1cc(Nc2ccnc(C(C)(F)F)n2)c(-c2csc(N3CCC3)n2)cn1] LCMS m/z = 431.1 [M + H]⁺; ¹H NMR (400 MHz, DMSO-d₆) δ ppm: 11.93 (s, 1H), 10.47 (s, 1H), 9.15 (s, 1H), 8.65-8.52 (m, 2H), 7.38 (s, 1H), 6.99 (s, 1H), 4.17 (t, J = 7.5 Hz, 4H), 2.14- 2.05 (m, 8H). |
| 789 | N-(5-(2-(2-oxa-6-azaspiro[3.3]heptan-6-yl)thiazol-4-yl)-4-((2-(1,1-difluoroethyl)pyrimidin- 4-yl)amino)pyridin-2-yl)acetamide 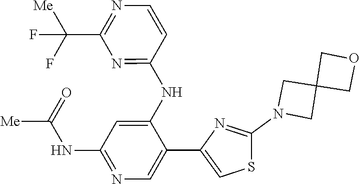 [SMILES: CC(=O)Nc1cc(Nc2ccnc(C(C)(F)F)n2)c(-c2csc(N3CC4(COC4)C3)n2)cn1] LCMS m/z = 474.1 [M + H]⁺; ¹H NMR (500 MHz, DMSO-d₆) δ ppm: 11.88 (s, 1H), 10.48 (s, 1H), 9.18 (s, 1H), 8.65 (s, 1H), 8.60 (d, J = 5.8 Hz, 1H), 7.40 (s, 1H), 7.04 (d, J = 6.0 Hz, 1H), 4.78 (s, 4H), 4.37 (s, 4H), 2.17-2.07 (m, 6H). |
| 790 | N-(5-(2-cyanothiazol-4-yl)-4-((2-(1,1-difluoroethyl)pyrimidin-4-yl)amino)pyridin-2- yl)acetamide 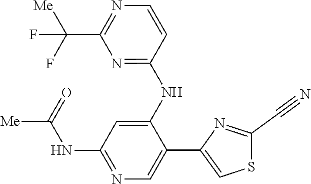 [SMILES: CC(=O)Nc1cc(Nc2ccnc(C(C)(F)F)n2)c(-c2csc(C#N)n2)cn1] LCMS m/z = 402.2 [M + H]⁺; ¹H NMR (400 MHz, CDCl₃) δ ppm: 10.72 (s, 1H), 9.45 (s, 1H), 8.61 (d, J = 5.6 Hz, 1H), 8.56 (br s, 1H), 8.37 (br s, 1H), 7.98 (s, 1H), 6.95 (d, J = 6.0 Hz, 1H), 2.27 (s, 3H), 2.18 (t, J = 18.8 Hz, 3H). |
| 791 | N-(4-((2-(1,1-difluoroethyl)-6-methylpyrimidin-4-yl)amino)-5-(5-(pyrrolidin-1- ylmethyl)pyrazin-2-yl)pyridin-2-yl)acetamide 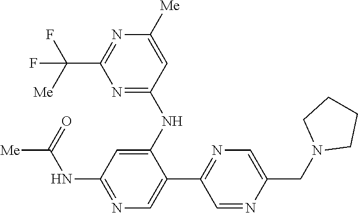 [SMILES: CC(=O)Nc1cc(Nc2cc(C)nc(C(C)(F)F)n2)c(-c2cnc(CN3CCCC3)cn2)cn1] LCMS m/z = 469.3 [M + H]⁺; ¹H NMR (400 MHz, CDCl₃) δ ppm: 11.87 (s, 1H), 9.30 (s, 1H), 9.04 (s, 1H), 8.75 (s, 1H), 8.64 (s, 1H), 7.98 (s, 1H), 6.79 (s, 1H), 3.92 (s, 2H), 2.68 (s, 4H), 2.55 (s, 3H), 2.26 (s, 3H), 2.16 (t, J = 18.8 Hz, 3H), 1.96-1.87 (m, 4H). |
| 792 | N-(4-((2-(1,1-difluoroethyl)-6-methylpyrimidin-4-yl)amino)-5-(5-((4-methylpiperazin-1- yl)methyl)pyrazin-2-yl)pyridin-2-yl)acetamide 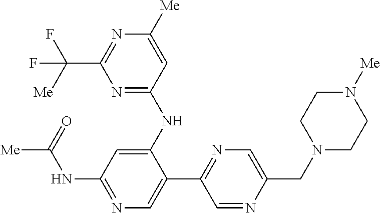 [SMILES: CC(=O)Nc1cc(Nc2cc(C)nc(C(C)(F)F)n2)c(-c2cnc(CN3CCN(C)CC3)cn2)cn1] LCMS m/z = 498.3 [M + H]⁺; ¹H NMR (500 MHz, MeOH-d₄) δ ppm: 9.19 (s, 1H), 9.10 (s, 1H), 8.79 (s, 1H), 8.75 (s, 1H), 6.98 (s, 1H), 3.80 (s, 2H), 2.64-2.46 (m, 11H), 2.32 (s, 3H), 2.22 (s, 3H), 2.07 (t, J = 19.0 Hz, 3H). |
| 793 | N-(4-((2-(1,1-difluoroethyl)-6-methylpyrimidin-4-yl)amino)-5-(5- (morpholinomethyl)pyrazin-2-yl)pyridin-2-yl)acetamide 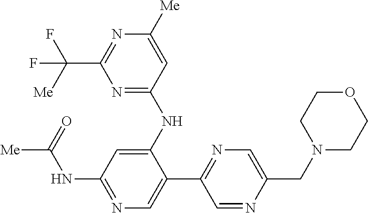 [SMILES: CC(=O)Nc1cc(Nc2cc(C)nc(C(C)(F)F)n2)c(-c2cnc(CN3CCOCC3)cn2)cn1] LCMS m/z = 485.3 [M + H]⁺; ¹H NMR (500 MHz, CDCl₃) δ ppm: 11.81 (s, 1H), 9.27 (s, 1H), 9.05 (s, 1H), 8.74 (s, 1H), 8.64 (s, 1H), 8.03 (s, 1H), 6.82 (s, 1H), 3.78-3.76 (m, 6H), 2.60- 2.58 (m, 4H), 2.55 (s, 3H), 2.19 (s, 3H), 2.13 (t, J = 19.5 Hz, 3H). |
| 794 | 1-(4-((2-(1,1-difluoroethyl)pyrimidin-4-yl)amino)-5-(5-methyl-4,5,6,7- tetrahydropyrazolo[1,5-a]pyrazin-2-yl)pyridin-2-yl)-3-methylurea 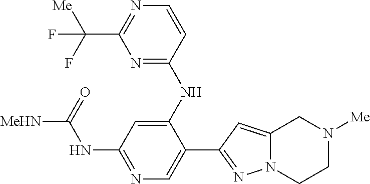 [SMILES: CNC(=O)Nc1cc(Nc2ccnc(C(C)(F)F)n2)c(-c2cc3n(n2)CCN(C)C3)cn1] LCMS m/z = 444.2 [M + H]⁺; ¹H NMR (400 MHz, CDCl₃) δ ppm: 11.66 (s, 1H), 9.23 (s, 1H), 8.49 (d, J = 5.6 Hz, 1H), 8.36 (s, 1H), 8.31 (s, 1H), 6.76 (d, J = 5.6 Hz, 1H), 6.34 (s, 1H), 4.29 (t, J = 5.6 Hz, 2H), 3.72 (s, 2H), 2.99-2.95 (m, 5H), 2.54 (s, 3H), 2.07 (t, J = 18.4 Hz, 3H). |
| 795 | 1-(4-((2-(1,1-difluoroethyl)pyrimidin-4-yl)amino)-5-(5-methyl-4,5,6,7- tetrahydrothiazolo[5,4-c]pyridin-2-yl)pyridin-2-yl)-3-methylurea 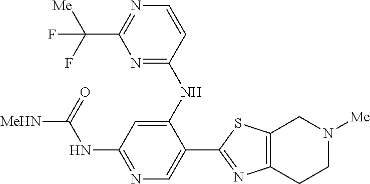 [SMILES: CNC(=O)Nc1cc(Nc2ccnc(C(C)(F)F)n2)c(-c2nc3c(s2)CN(C)CC3)cn1] LCMS m/z = 461.2 [M + H]⁺; ¹H NMR (400 MHz, DMSO-d₆) δ ppm: 12.07 (s, 1H), 9.53 (s, 1H), 8.56-8.62 (m, 3H), 7.66 (s, 1H), 7.15 (d, J = 5.6 Hz, 1H), 3.65 (s, 2H), 2.92 (s, 2H), 2.72-2.77 (m, 5H), 2.41 (s, 3H) , 2.12 (brt, J = 19.2 Hz, 3H). |
| 796 | N-(4-((2-(1,1-difluoroethyl)pyrimidin-4-yl)amino)-5-(5-methyl-4,5,6,7- tetrahydrothiazolo[4,5-c]pyridin-2-yl)pyridin-2-yl)formamide 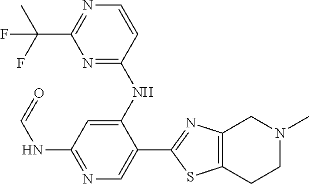 LCMS m/z = 461.2 [M + H]⁺; ¹H NMR (400 MHz, DMSO-d₆) δ ppm: |
| 797 | N-(4-((2-(1,1-difluoroethyl)pyrimidin-4-yl)amino)-5-(5-methyl-4,5,6,7- tetrahydrothiazolo[4,5-c]pyridin-2-yl)pyridin-2-yl)acetamide 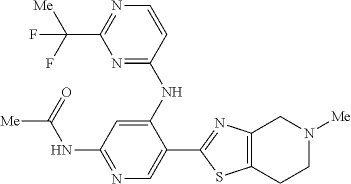 [SMILES: CC(=O)Nc1cc(Nc2ccnc(C(C)(F)F)n2)c(-c2nc3c(s2)CCN(C)C3)cn1] LCMS m/z = 446.2 [M + H]⁺; ¹H NMR (400 MHz, CDCl₃) δ ppm: 12.60 (s, 1H), 9.50 (s, 1H), 8.54-8.56 (m, 2H), 7.91 (s, 1H), 6.85 (d, J = 6.0 Hz, 1H), 3.85 (s, 2H), 2.95-3.05 (m, 4H), 2.65 (s, 3H), 2.16-2.25 (m, 6H). |
| 798 | N-(4-((2-(1,1-difluoroethyl)pyrimidin-4-yl)amino)-5-(5-methyl-4,5,6,7- tetrahydrothiazolo[5,4-c]pyridin-2-yl)pyridin-2-yl)acetamide 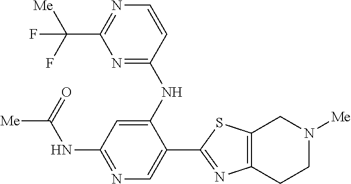 [SMILES: CC(=O)Nc1cc(Nc2ccnc(C(C)(F)F)n2)c(-c2nc3c(s2)CN(C)CC3)cn1] LCMS m/z = 446.1 [M + H]⁺; ¹H NMR (400 MHz, MeOH-d₄) δ ppm: 9.31 (s, 1H), 8.46 (s, 1H), 8.42 (d, J = 6.0 Hz, 1H), 6.82 (d, J = 6.0 Hz, 1H), 3.69 (s, 2H), 2.92-2.89 (m, 4H), 2.53 (s, 3H), 2.19-2.09 (m, 6H). |
| 799 | N-(4-((2-(1,1-difluoroethyl)-6-ethylpyrimidin-4-yl)amino)-5-(pyrimidin-4-yl)pyridin-2- yl)acetamide 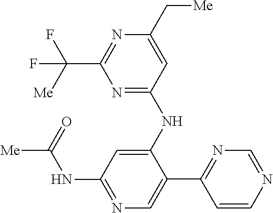 [SMILES: CCc1cc(Nc2cc(NC(C)=O)ncc2-c2ccncn2)nc(C(C)(F)F)n1] LCMS m/z = 400.1 [M + H]⁺; ¹H NMR (400 MHz, CDCl₃) δ ppm: 12.51 (s, 1H), 9.38 (s, 1H), 9.31 (s, 1H), 8.84 (d, J = 5.6 Hz, 1H), 8.67 (s, 1H), 7.91 (s, 1H), 7.79 (d, J = 4.8 Hz, 1H),6.81 (s, 1H), 2.83 (q, J = 7.6 Hz, 2H), 2.26 (s, 3H), 2.17 (t, J = 19.2 Hz, 3H), 1.35 (t, J = 7.6 Hz, 3H). |
| 800 | N-(4-((2-(2-fluoropropan-2-yl)-6-methylpyrimidin-4-yl)amino)-5-(pyrimidin-4-yl)pyridin-2- yl)acetamide 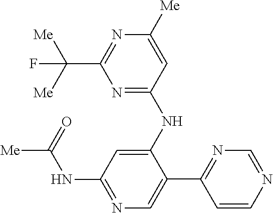 [SMILES: CC(=O)Nc1cc(Nc2cc(C)nc(C(C)(C)F)n2)c(-c2ccncn2)cn1] LCMS m/z = 382.2 [M + H]⁺; ¹H NMR (400 MHz, MeOH-d₄) δ ppm: 9.47 (s, 1H), 9.32 (s, 1H), 8.82-8.85 (m, 2H), 8.07-8.09 (m, 1H), 6.90 (s, 1H), 2.49 (s, 3H), 2.22 (s, 3H), 1.84 (s, 3H), 1.79 (s, 3H). |
| 801 | N-(4-((6-ethyl-2-(2-fluoropropan-2-yl)pyrimidin-4-yl)amino)-5-(pyrimidin-4-yl)pyridin-2- yl)acetamide 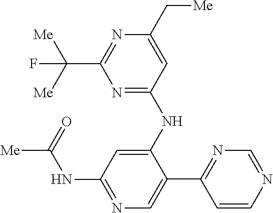 [SMILES: CCc1cc(Nc2cc(NC(C)=O)ncc2-c2ccncn2)nc(C(C)(C)F)n1] LCMS m/z = 396.2 [M + H]⁺; ¹H NMR (400 MHz, DMSO-d₆) δ ppm: 12.19 (s, 1H), 10.63 (s, 1H), 9.31-9.35 (m, 2H), 8.88 (d, J = 4.4 Hz, 2H), 8.17 (d, J = 5.6 Hz 1H), 6.96 (s, 1H), 2.70 (q, J = 7.2 Hz, 2H), 2.14 (s, 3H), 1.77 (s, 3H), 1.72 (s, 3H), 1.25 (t, J = 7.6 Hz, 3H). |
| 802 | N-(4-((2-(1,1-difluoroethyl)-6-methylpyrimidin-4-yl)amino)-5-(5-(2- (dimethylamino)propan-2-yl)-1-methyl-1H-pyrazol-3-yl)pyridin-2-yl)acetamide 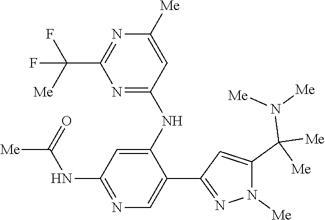 [SMILES: CC(=O)Nc1cc(Nc2cc(C)nc(C(C)(F)F)n2)c(-c2cc(C(C)(C)N(C)C)n(C)n2)cn1] LCMS m/z = 473.3 [M + H]⁺; ¹H NMR (400 MHz, DMSO-d₆) δ ppm: 11.63 (s, 1H), 10.44 (s, 1H), 9.32 (s, 1H), 8.68 (s, 1H), 7.04 (s, 1H), 6.83 (s, 1H), 4.19 (s, 3H), 2.45 (s, 3H), 2.08- 2.18 (m, 12H), 1.40 (s, 6H). |
| 803 | N-(4-((2-(1,1-difluoroethyl)-6-methylpyrimidin-4-yl)amino)-5-(1-methyl-5-(pyrrolidin-1- ylmethyl)-1H-pyrazol-3-yl)pyridin-2-yl)acetamide 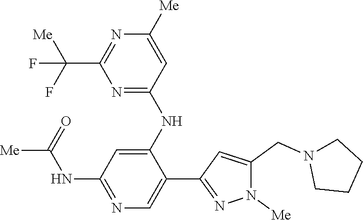 [SMILES: CC(=O)Nc1cc(Nc2cc(C)nc(C(C)(F)F)n2)c(-c2cc(CN3CCCC3)n(C)n2)cn1] ¹H NMR (400 MHz, CDCl₃) δ ppm: 11.40 (s, 1H), 9.35 (s, 1H), 8.45 (s, 1H), 7.88 (s, 1H), 6.74 (s, 1H), 6.56 (s, 1H), 4.03 (s, 3H), 3.73 (s, 2H), 2.62-2.61 (m, 4H), 2.55 (s, 3H), 2.23 (s, 3H), 2.20 (t, J = 18.8 Hz, 3H), 1.85-1.84 (m, 4H). |
| 804 | N-(4-((2-(1,1-difluoroethyl)-6-methylpyrimidin-4-yl)amino)-5-(1-methyl-5-((4- methylpiperazin-1-yl)methyl)-1H-pyrazol-3-yl)pyridin-2-yl)acetamide 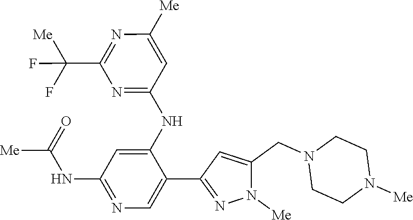 [SMILES: CC(=O)Nc1cc(Nc2cc(C)nc(C(C)(F)F)n2)c(-c2cc(CN3CCN(C)CC3)n(C)n2)cn1] LCMS m/z = 500.4 [M + H]⁺; ¹H NMR (500 MHz, CDCl₃) δ ppm: 11.39 (s, 1H), 9.33 (s, 1H), 8.44 (s, 1H), 8.00 (s, 1H), 6.75 (s, 1H), 6.53 (s, 1H), 4.01 (s, 3H), 3.57 (s, 2H), 2.74-2.35 (m, 11H), 2.31 (s, 3H), 2.25-2.15(m, 6H). |
| 805 | N-(4′-((2-(1,1-difluoroethyl)-6-methylpyrimidin-4-yl)amino)-4-(pyrrolidin-1-ylmethyl)-[2,3′- bipyridin]-6′-yl)acetamide 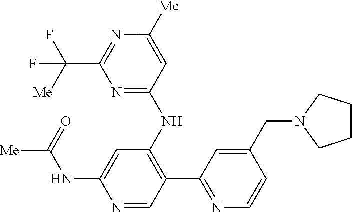 [SMILES: CC(=O)Nc1cc(Nc2cc(C)nc(C(C)(F)F)n2)c(-c2cc(CN3CCCC3)ccn2)cn1] LCMS m/z = 468.2 [M + H]⁺; ¹H NMR (500 MHz, DMSO-d₆) δ ppm: 12.72 (s, 1H), 10.56 (s, 1H), 9.22 (s, 1H), 8.76 (s, 1H), 8.70 (d, J = 5.0 Hz, 1H), 7.95 (s, 1H), 7.41 (d, J = 5.0 Hz, 1H), 7.06 (s, 1H), 3.70 (s, 2H), 3.30 (s, 3H), 2.42 (s, 3H), 2.16-2.01 (m, 7H), 1.72 (s, 4H). |
| 806 | N-(4′-((2-(1,1-difluoroethyl)-6-methylpyrimidin-4-yl)amino)-4-(morpholinomethyl)-[2,3′- bipyridin]-6′-yl)acetamide 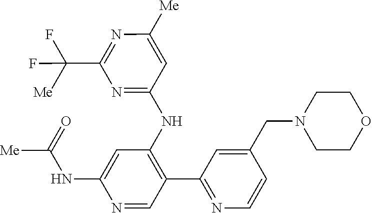 [SMILES: CC(=O)Nc1cc(Nc2cc(C)nc(C(C)(F)F)n2)c(-c2cc(CN3CCOCC3)ccn2)cn1] LCMS m/z = 484.2 [M + H]⁺; ¹H NMR (400 MHz, CDCl₃) δ ppm: 12.68 (s, 1H), 9.31 (s, 1H), 8.62 (d, J = 7.2 Hz, 1H), 8.58 (s, 1H), 8.00 (s, 1H), 7.79 (s, 1H), 7.32 (d, J = 5.2 Hz, 1H), 6.75 (s, 1H), 3.76-3.73 (m, 4H), 3.59 (s, 2H), 2.53-2.49 (m, 7H), 2.25 (s, 3H), 2.17 (t, J = 19.0 Hz, 3H). |
| 807 | N-(4-((2-(1,1-difluoroethyl)-6-methylpyrimidin-4-yl)amino)-5-(5- ((dimethylamino)methyl)pyrazin-2-yl)pyridin-2-yl)acetamide 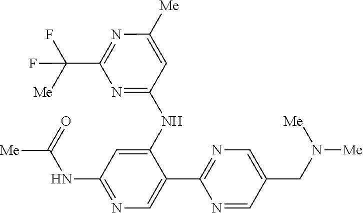 [SMILES: CC(=O)Nc1cc(Nc2cc(C)nc(C(C)(F)F)n2)c(-c2ncc(CN(C)C)cn2)cn1] LCMS m/z = 443.2 [M + H]⁺; ¹H NMR (400 MHz, CDCl₃) δ ppm: 11.95 (s, 1H), 9.29 (s, 1H), 9.04 (d, J = 1.6 Hz, 1H), 8.72 (s, 1H), 8.65 (s, 1H), 8.06 (s, 1H), 6.82 (s, 1H), 3.71 (s, 2H), 2.54 (s, 3H), 2.37 (s, 6H), 2.26 (s, 3H), 2.16 (t, J = 18.8 Hz, 3H). |
| 808 | N-(4-((2-(1,1-difluoroethyl)-6-methoxypyrimidin-4-yl)amino)pyridin-2-yl)acetamide 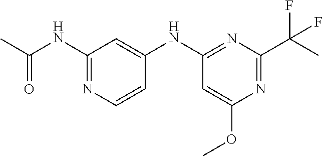 [SMILES: COc1cc(Nc2ccnc(NC(C)=O)c2)nc(C(C)(F)F)n1] LCMS m/z = 324.0 [M + H]+ |
| 809 | N-(4-((2-(1,1-difluoroethyl)-6-(2-methoxyethoxy)pyrimidin-4-yl)amino)pyridin-2- yl)acetamide 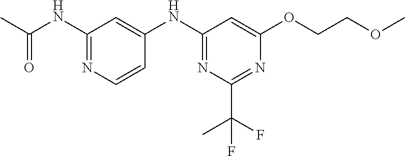 [SMILES: COCCOc1cc(Nc2ccnc(NC(C)=O)c2)nc(C(C)(F)F)n1] LCMS m/z = 368.0 [M + H]+ |
| 810 | rel-N-(5-((1R,2S)-2-cyanocyclopropyl)-4-((2-(1,1-difluoroethyl)-6-methylpyrimidin-4- yl)amino)pyridin-2-yl)acetamide 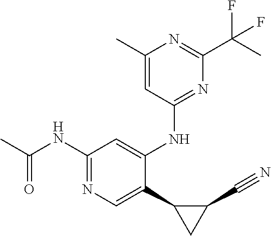 [SMILES: CC(=O)Nc1cc(Nc2cc(C)nc(C(C)(F)F)n2)c([C@@H]2C[C@@H]2C#N)cn1] LCMS m/z = 373 [M + H]+ |
| 811 | N-(4-((2-(1,1-difluoroethyl)-6-methylpyrimidin-4-yl)amino)-5-(1-(difluoromethyl)-1H- pyrazol-3-yl)pyridin-2-yl)acetamide 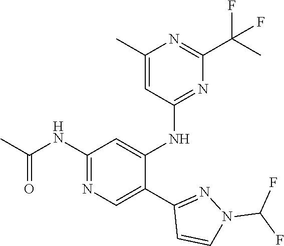 [SMILES: CC(=O)Nc1cc(Nc2cc(C)nc(C(C)(F)F)n2)c(-c2ccn(C(F)F)n2)cn1] LCMS m/z = 424.1 [M + H]+ |
| 812 | N-(4-((2-(1,1-difluoroethyl)-6-methylpyrimidin-4-yl)amino)-5-(1-methyl-1H-pyrazol-4- yl)pyridin-2-yl)acetamide 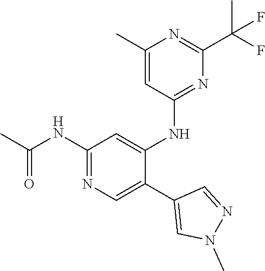 [SMILES: CC(=O)Nc1cc(Nc2cc(C)nc(C(C)(F)F)n2)c(-c2cnn(C)c2)cn1] LCMS m/z = 388.1 [M + H]+ |
| 813 | N-(4-((2-(1,1-difluoroethyl)-6-methylpyrimidin-4-yl)amino)-5-(2,2-dimethyl-2,3-dihydro- [1,4]dioxino[2,3-b]pyridin-6-yl)pyridin-2-yl)acetamide 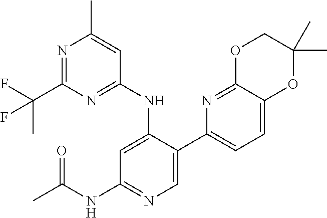 [SMILES: CC(=O)Nc1cc(Nc2cc(C)nc(C(C)(F)F)n2)c(-c2ccc3c(n2)OCC(C)(C)O3)cn1] LCMS m/z = 471.2 [M + H]+ |
| 814 | N-(4-((2-(1,1-difluoroethyl)-6-methylpyrimidin-4-yl)amino)-5-(5-fluoro-1-methyl-1H- pyrazol-3-yl)pyridin-2-yl)acetamide 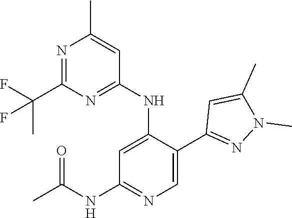 LCMS m/z = 406.1 [M + H]+ |
| 815 | N-(4-((2-(1,1-difluoroethyl)-6-methylpyrimidin-4-yl)amino)-5-(5-methoxypyrazin-2- yl)pyridin-2-yl)acetamide 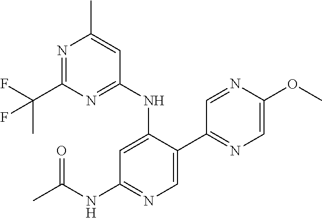 [SMILES: COc1cnc(-c2cnc(NC(C)=O)cc2Nc2cc(C)nc(C(C)(F)F)n2)cn1] LCMS m/z = 416.2 [M + H]+ |
| 816 | N-(4-((2-(1,1-difluoroethyl)-6-ethylpyrimidin-4-yl)amino)-5-fluoropyridin-2-yl)acetamide 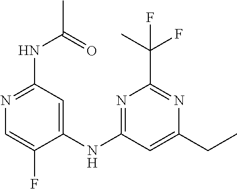 [SMILES: CCc1cc(Nc2cc(NC(C)=O)ncc2F)nc(C(C)(F)F)n1] LCMS m/z = 340.1 [M + H]+ |
| 817 | N-(4-((2-(1,1-difluoroethyl)-6-isopropylpyrimidin-4-yl)amino)-5-fluoropyridin-2- yl)acetamide 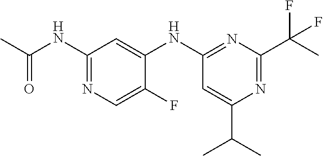 [SMILES: CC(=O)Nc1cc(Nc2cc(C(C)C)nc(C(C)(F)F)n2)c(F)cn1] LCMS m/z = 354.1 [M + H]+ |
| 818 | N-(4-((6-cyclopropoxy-2-(1,1-difluoroethyl)pyrimidin-4-yl)amino)-5-fluoropyridin-2- yl)acetamide 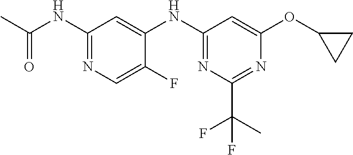 [SMILES: CC(=O)Nc1cc(Nc2cc(OC3CC3)nc(C(C)(F)F)n2)c(F)cn1] LCMS m/z = 368.1 [M + H]+ |
| 819 | N-(4-((2-(1,1-difluoroethyl)-6-(1-isopropyl-1H-pyrazol-4-yl)pyrimidin-4-yl)amino)-5- fluoropyridin-2-yl)acetamide 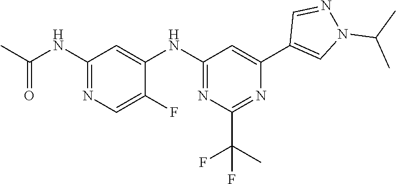 LCMS m/z = 420.2 [M + H]+ |
| 820 | N-(4-((6-(1,1-difluoroethyl)pyridin-2-yl)amino)-5-(2,2-dimethyl-2,3-dihydro- [1,4]dioxino[2,3-b]pyridin-6-yl)pyridin-2-yl)acetamide 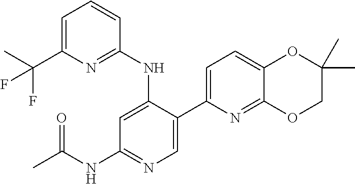 [SMILES: CC(=O)Nc1cc(Nc2cccc(C(C)(F)F)n2)c(-c2ccc3c(n2)OCC(C)(C)O3)cn1] LCMS m/z = 456.2 [M + H]+ |
| 821 | N-(4-((2-(1,1-difluoroethyl)-6-ethylpyrimidin-4-yl)amino)-5-(2,2-dimethyl-2,3-dihydro- [1,4]dioxino[2,3-b]pyridin-6-yl)pyridin-2-yl)acetamide 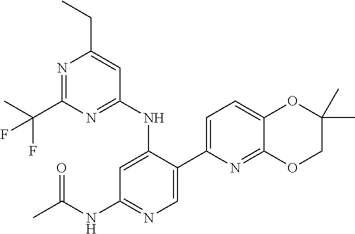 [SMILES: CCc1cc(Nc2cc(NC(C)=O)ncc2-c2ccc3c(n2)OCC(C)(C)O3)nc(C(C)(F)F)n1] LCMS m/z = 485.2 [M + H]+ |
| 822 | N-(4-((2-(1,1-difluoroethyl)-6-isopropylpyrimidin-4-yl)amino)-5-(2,2-dimethyl-2,3-dihydro- [1,4]dioxino[2,3-b]pyridin-6-yl)pyridin-2-yl)acetamide 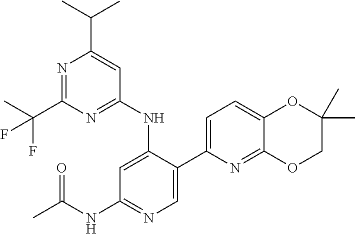 [SMILES: CC(=O)Nc1cc(Nc2cc(C(C)C)nc(C(C)(F)F)n2)c(-c2ccc3c(n2)OCC(C)(C)O3)cn1] LCMS m/z = 499.2 [M + H]+ |
| 823 | N-(4-((2-(1,1-difluoroethyl)-6-ethylpyrimidin-4-yl)amino)-5-(pyrazin-2-yl)pyridin-2- yl)acetamide 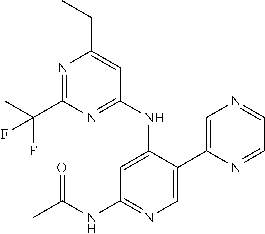 [SMILES: CCc1cc(Nc2cc(NC(C)=O)ncc2-c2cnccn2)nc(C(C)(F)F)n1] LCMS m/z = 400.2 [M + H]+ |
| 824 | N-(4-((6-cyclopropoxy-2-(1,1-difluoroethyl)pyrimidin-4-yl)amino)-5-(pyrazin-2-yl)pyridin- 2-yl)acetamide 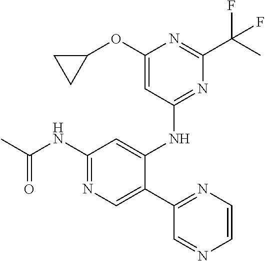 [SMILES: CC(=O)Nc1cc(Nc2cc(OC3CC3)nc(C(C)(F)F)n2)c(-c2cnccn2)cn1] LCMS m/z = 428.2 [M + H]+ |
| 825 | N-(4-((2-(1,1-difluoroethyl)pyrimidin-4-yl)amino)-5-(5-(difluoromethoxy)pyrimidin-2- yl)pyridin-2-yl)acetamide 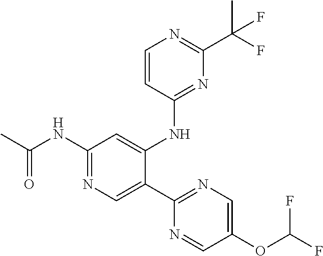 [SMILES: CC(=O)Nc1cc(Nc2ccnc(C(C)(F)F)n2)c(-c2ncc(OC(F)F)cn2)cn1] LCMS m/z = 438.1 [M + H]+ |
| 826 | N-(4′-((2-(1,1-difluoroethyl)pyrimidin-4-yl)amino)-5-(difluoromethoxy)-[2,3′-bipyridin]-6′- yl)acetamide 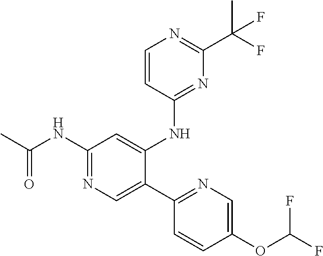 [SMILES: CC(=O)Nc1cc(Nc2ccnc(C(C)(F)F)n2)c(-c2ccc(OC(F)F)cn2)cn1] LCMS m/z = 437.1 [M + H]+ |
| 827 | N-(4′-((2-(1,1-difluoroethyl)-6-methylpyrimidin-4-yl)amino)-5-(difluoromethyl)-[2,3′- bipyridin]-6′-yl)acetamide 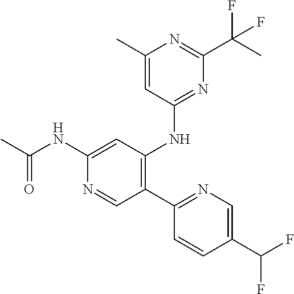 [SMILES: CC(=O)Nc1cc(Nc2cc(C)nc(C(C)(F)F)n2)c(-c2ccc(C(F)F)cn2)cn1] LCMS m/z = 435.1 [M + H]+ |
| 828 | N-(4-((2-(1,1-difluoroethyl)pyrimidin-4-yl)amino)-5-(5-methoxypyrazin-2-yl)pyridin-2- yl)acetamide 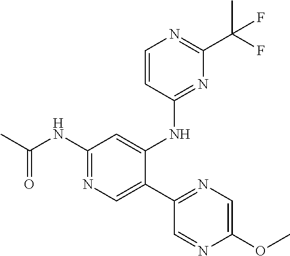 [SMILES: COc1cnc(-c2cnc(NC(C)=O)cc2Nc2ccnc(C(C)(F)F)n2)cn1] LCMS m/z = 402.1 [M + H]+ |
| 829 | N-(4-((2-(1,1-difluoroethyl)-6-ethylpyrimidin-4-yl)amino)-5-(5-methoxypyrazin-2- yl)pyridin-2-yl)acetamide 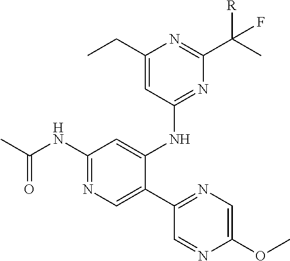 LCMS m/z = 430.2 [M + H]+ |
| 830 | N-(4-((6-cyclopropoxy-2-(1,1-difluoroethyl)pyrimidin-4-yl)amino)-5-(5-methoxypyrazin-2- yl)pyridin-2-yl)acetamide 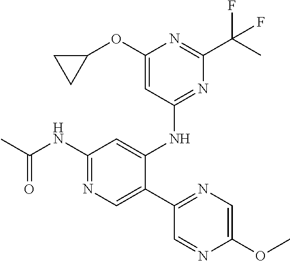 [SMILES: COc1cnc(-c2cnc(NC(C)=O)cc2Nc2cc(OC3CC3)nc(C(C)(F)F)n2)cn1] LCMS m/z = 458.2 [M + H]+ |
| 831 | N-(4-((2-(1,1-difluoroethyl)-6-ethylpyrimidin-4-yl)amino)-5-(1-methyl-6-oxo-1,6- dihydropyridazin-3-yl)pyridin-2-yl)acetamide 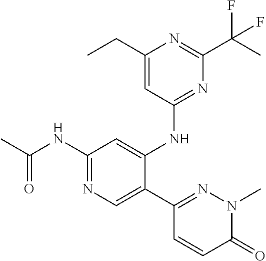 [SMILES: CCc1cc(Nc2cc(NC(C)=O)ncc2-c2ccc(=O)n(C)n2)nc(C(C)(F)F)n1] LCMS m/z = 430.2 [M + H]+ |
| 832 | N-(4-((2-(1,1-difluoroethyl)-6-isopropylpyrimidin-4-yl)amino)-5-(6-methoxypyridazin-3- yl)pyridin-2-yl)acetamide 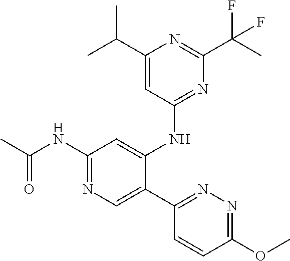 [SMILES: COc1ccc(-c2cnc(NC(C)=O)cc2Nc2cc(C(C)C)nc(C(C)(F)F)n2)nn1] LCMS m/z = 444.2 [M + H]+ |
| 833 | N-(4-((6-cyclopropoxy-2-(1,1-difluoroethyl)pyrimidin-4-yl)amino)-5-(6-methoxypyridazin- 3-yl)pyridin-2-yl)acetamide 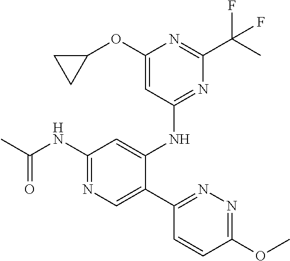 [SMILES: COc1ccc(-c2cnc(NC(C)=O)cc2Nc2cc(OC3CC3)nc(C(C)(F)F)n2)nn1] LCMS m/z = 458.2 [M + H]+ |
| 834 | N-(4-((2-(1,1-difluoroethyl)-6-methylpyrimidin-4-yl)amino)-5-(5-(methoxymethyl)pyrazin- 2-yl)pyridin-2-yl)acetamide 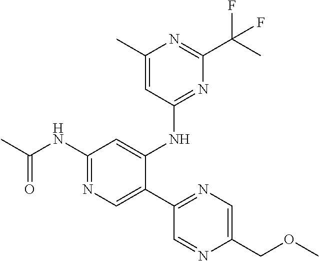 [SMILES: COCc1cnc(-c2cnc(NC(C)=O)cc2Nc2cc(C)nc(C(C)(F)F)n2)cn1] LCMS m/z = 430.2 [M + H]+ |
| 835 | N-(4-((2-(2-fluoropropan-2-yl)pyrimidin-4-yl)amino)-5-(6-methoxypyridazin-3-yl)pyridin-2- yl)acetamide 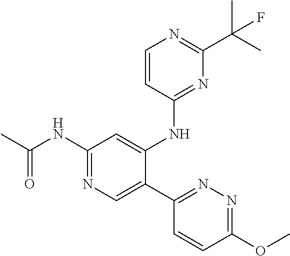 [SMILES: COc1ccc(-c2cnc(NC(C)=O)cc2Nc2ccnc(C(C)(C)F)n2)nn1] LCMS m/z = 398.2 [M + H]+ |
| 836 | N-(4-((2-(1,1-difluoroethyl)-6-ethylpyrimidin-4-yl)amino)-5-(5-(methoxymethyl)pyrazin-2- yl)pyridin-2-yl)acetamide 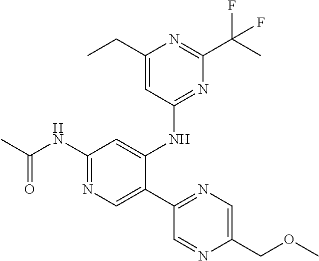 [SMILES: CCc1cc(Nc2cc(NC(C)=O)ncc2-c2cnc(COC)cn2)nc(C(C)(F)F)n1] LCMS m/z = 444.2 [M + H]+ |
| 837 | N-(4-((2-(1, 1-difluoroethyl)-6-methylpyrimidin-4-yl)amino)-5-(2-(1,1- difluoroethyl)pyrimidin-4-yl)pyridin-2-yl)acetamide 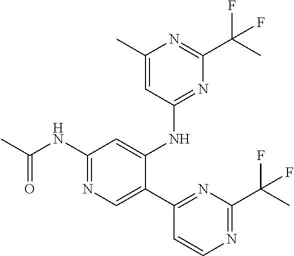 [SMILES: CC(=O)Nc1cc(Nc2cc(C)nc(C(C)(F)F)n2)c(-c2ccnc(C(C)(F)F)n2)cn1] LCMS m/z = 450.2 [M + H]+ |
| 838 | N-(5-(5-cyanopyrazin-2-yl)-4-((2-(1,1-difluoroethyl)-6-methylpyrimidin-4- yl)amino)pyridin-2-yl)acetamide 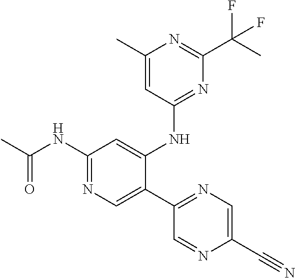 [SMILES: CC(=O)Nc1cc(Nc2cc(C)nc(C(C)(F)F)n2)c(-c2cnc(C#N)cn2)cn1] LCMS m/z = 411.1 [M + H]+ |
| 839 | N-(4′-((2-(1,1-difluoroethyl)-6-ethylpyrimidin-4-yl)amino)-5-(2-hydroxypropan-2-yl)-[2,3′- bipyridin]-6′-yl)acetamide 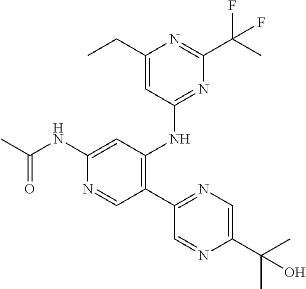 [SMILES: CCc1cc(Nc2cc(NC(C)=O)ncc2-c2cnc(C(C)(C)O)cn2)nc(C(C)(F)F)n1] LCMS m/z = 457.2 [M + H]+ |
| 840 | N-(5-(5-(1,4-dioxan-2-yl)pyrimidin-2-yl)-4-((2-(1,1-difluoroethyl)-6-ethylpyrimidin-4- yl)amino)pyridin-2-yl)acetamide 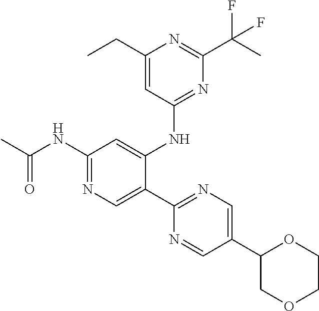 [SMILES: CCc1cc(Nc2cc(NC(C)=O)ncc2-c2ncc(C3COCCO3)cn2)nc(C(C)(F)F)n1] LCMS m/z = 486.2 [M + H]+ |
| 841 | N-(4-((2-(1,1-difluoroethyl)pyrimidin-4-yl)amino)-5-(6-(dimethylamino)pyridazin-3- yl)pyridin-2-yl)acetamide 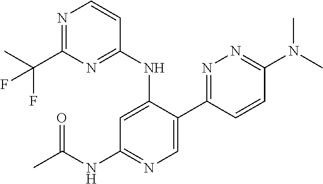 [SMILES: CC(=O)Nc1cc(Nc2ccnc(C(C)(F)F)n2)c(-c2ccc(N(C)C)nn2)cn1] LCMS m/z = 415.2 [M + H]+ |
| 842 | N-(4-((2-(1,1-difluoroethyl)pyrimidin-4-yl)amino)-5-(5-(dimethylamino)pyrazin-2- yl)pyridin-2-yl)acetamide 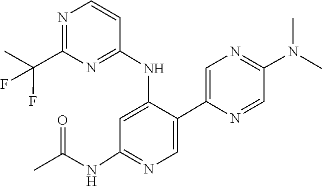 [SMILES: CC(=O)Nc1cc(Nc2ccnc(C(C)(F)F)n2)c(-c2cnc(N(C)C)cn2)cn1] LCMS m/z = 415.2 [M + H]+ |
| 843 | N-(4-((2-(1,1-difluoroethyl)pyrimidin-4-yl)amino)-5-(5-(pyrrolidin-1-yl)pyrazin-2- yl)pyridin-2-yl)acetamide 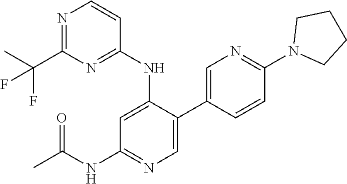 [SMILES: CC(=O)Nc1cc(Nc2ccnc(C(C)(F)F)n2)c(-c2ccc(N3CCCC3)nc2)cn1] LCMS m/z = 441.2 [M + H]+ |
| 844 | N-(4-((2-(1,1-difluoroethyl)pyrimidin-4-yl)amino)-5-(5-morpholinopyrazin-2-yl)pyridin-2- yl)acetamide 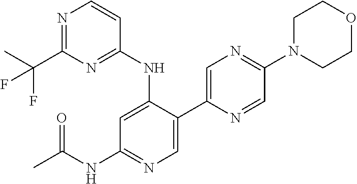 [SMILES: CC(=O)Nc1cc(Nc2ccnc(C(C)(F)F)n2)c(-c2cnc(N3CCOCC3)cn2)cn1] LCMS m/z = 457.2 [M + H]+ |
| 845 | N-(4-((2-(1,1-difluoroethyl)pyrimidin-4-yl)amino)-5-(6-(pyrrolidin-1-yl)pyridazin-3- yl)pyridin-2-yl)acetamide 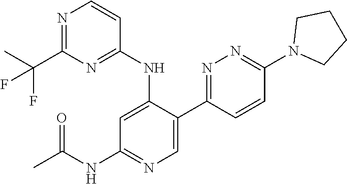 [SMILES: CC(=O)Nc1cc(Nc2ccnc(C(C)(F)F)n2)c(-c2ccc(N3CCCC3)nn2)cn1] LCMS m/z = 441.2 [M + H]+ |
| 846 | N-(4′-((2-(1,1-difluoroethyl)-6-ethylpyrimidin-4-yl)amino)-5-(methoxymethyl)-[2,3′- bipyridin]-6′-yl)acetamide 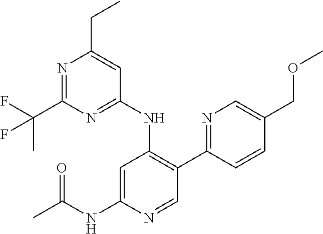 [SMILES: CCc1cc(Nc2cc(NC(C)=O)ncc2-c2ccc(COC)cn2)nc(C(C)(F)F)n1] LCMS m/z = 443.2 [M + H]+ |
| 847 | N-(5-(5-cyanopyrimidin-2-yl)-4-((2-(1,1-difluoroethyl)-6-ethylpyrimidin-4- yl)amino)pyridin-2-yl)acetamide 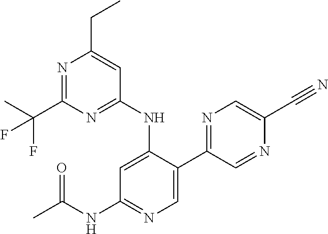 [SMILES: CCc1cc(Nc2cc(NC(C)=O)ncc2-c2cnc(C#N)cn2)nc(C(C)(F)F)n1] LCMS m/z = 425.2 [M + H]+ |
| 848 | N-(4-((2-(1,1-difluoroethyl)-6-ethylpyrimidin-4-yl)amino)-5-(5- (difluoromethoxy)pyrimidin-2-yl)pyridin-2-yl)acetamide 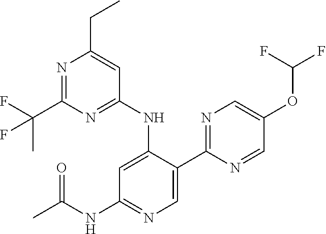 [SMILES: CCc1cc(Nc2cc(NC(C)=O)ncc2-c2ncc(OC(F)F)cn2)nc(C(C)(F)F)n1] LCMS m/z = 466.2 [M + H]+ |
| 849 | N-(4′-((2-(1,1-difluoroethyl)-6-ethylpyrimidin-4-yl)amino)-5-(difluoromethoxy)-[2,3′- bipyridin]-6′-yl)acetamide 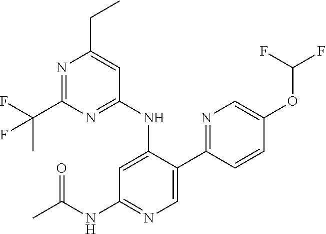 [SMILES: CCc1cc(Nc2cc(NC(C)=O)ncc2-c2ccc(OC(F)F)cn2)nc(C(C)(F)F)n1] LCMS m/z = 465.2 [M + H]+ |
| 850 | N-(5-(5-cyanopyrazin-2-yl)-4-((2-(1,1-difluoroethyl)pyrimidin-4-yl)amino)pyridin-2- yl)acetamide 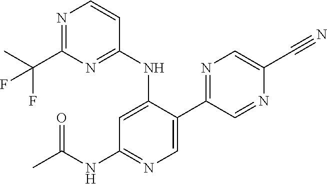 [SMILES: CC(=O)Nc1cc(Nc2ccnc(C(C)(F)F)n2)c(-c2cnc(C#N)cn2)cn1] LCMS m/z = 397.1 [M + H]+ |
| 851 | N-(4′-((2-(1,1-difluoroethyl)pyrimidin-4-yl)amino)-5-(methoxymethyl)-[2,3′-bipyridin]-6′- yl)acetamide 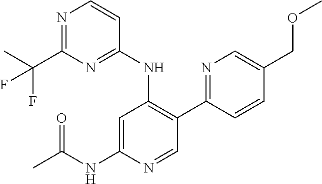 [SMILES: COCc1ccc(-c2cnc(NC(C)=O)cc2Nc2ccnc(C(C)(F)F)n2)nc1] LCMS m/z = 415.2 [M + H]+ |
| 852 | N-(4′-((2-(1,1-difluoroethyl)pyrimidin-4-yl)amino)-5-(2-hydroxypropan-2-yl)-[2,3′- bipyridin]-6′-yl)acetamide 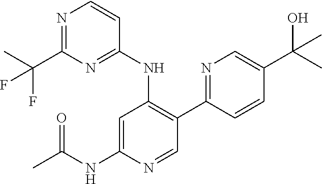 [SMILES: CC(=O)Nc1cc(Nc2ccnc(C(C)(F)F)n2)c(-c2ccc(C(C)(C)O)cn2)cn1] LCMS m/z = 429.2 [M + H]+ |
| 853 | N-(4-((2-(1,1-difluoroethyl)pyrimidin-4-yl)amino)-5-(5-(2-hydroxypropan-2-yl)pyrazin-2- yl)pyridin-2-yl)acetamide 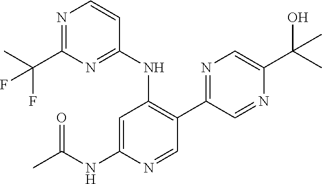 [SMILES: CC(=O)Nc1cc(Nc2ccnc(C(C)(F)F)n2)c(-c2cnc(C(C)(C)O)cn2)cn1] LCMS m/z = 430.2 [M + H]+ |
| 854 | N-(4-((2-(1,1-difluoroethyl)pyrimidin-4-yl)amino)-5-(5-(difluoromethoxy)pyrazin-2- yl)pyridin-2-yl)acetamide 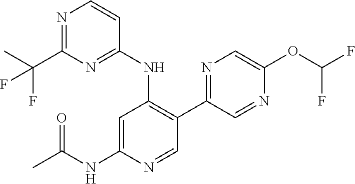 [SMILES: CC(=O)Nc1cc(Nc2ccnc(C(C)(F)F)n2)c(-c2cnc(OC(F)F)cn2)cn1] LCMS m/z = 437.1 [M + H]+ |
| 855 | N-(4-((2-(1,1-difluoroethyl)-6-ethylpyrimidin-4-yl)amino)-5-(5-(2-hydroxypropan-2- yl)pyrazin-2-yl)pyridin-2-yl)acetamide 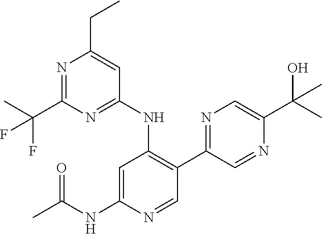 [SMILES: CCc1cc(Nc2cc(NC(C)=O)ncc2-c2cnc(C(C)(C)O)cn2)nc(C(C)(F)F)n1] LCMS m/z = 458.2 [M + H]+ |
| 856 | N-(4-((2-(1,1-difluoroethyl)-6-ethylpyrimidin-4-yl)amino)-5-(5-(dimethylamino)pyrazin-2- yl)pyridin-2-yl)acetamide 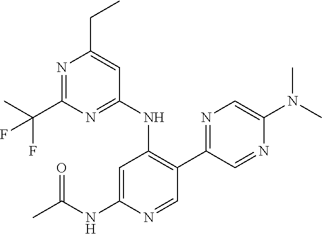 [SMILES: CCc1cc(Nc2cc(NC(C)=O)ncc2-c2cnc(N(C)C)cn2)nc(C(C)(F)F)n1] LCMS m/z = 443.2 [M + H]+ |
| 857 | N-(4′-((2-(1,1-difluoroethyl)pyrimidin-4-yl)amino)-5-(methylamino)-[2,3′-bipyridin]-6′- yl)acetamide 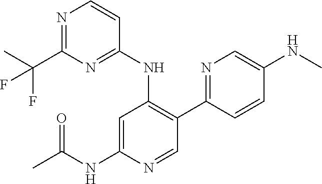 [SMILES: CNc1ccc(-c2cnc(NC(C)=O)cc2Nc2ccnc(C(C)(F)F)n2)nc1] LCMS m/z = 400.1 [M + H]+ |
| 858 | N-(4-((2-(1,1-difluoroethyl)pyrimidin-4-yl)amino)-5-(6-(difluoromethoxy)pyridazin-3- yl)pyridin-2-yl)acetamide 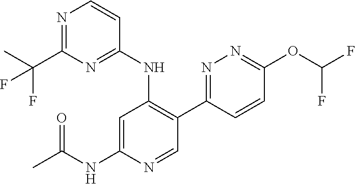 [SMILES: CC(=O)Nc1cc(Nc2ccnc(C(C)(F)F)n2)c(-c2ccc(OC(F)F)nn2)cn1] LCMS m/z = 438.1 [M + H]+ |
| 859 | N-(4′-((2-(1,1-difluoroethyl)-6-ethylpyrimidin-4-yl)amino)-5-(methylamino)-[2,3′- bipyridin]-6′-yl)acetamide 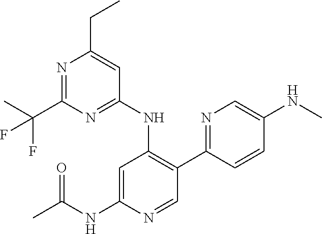 [SMILES: CCc1cc(Nc2cc(NC(C)=O)ncc2-c2ccc(NC)cn2)nc(C(C)(F)F)n1] LCMS m/z = 428.2 [M + H]+ |
| 860 | N-(4-((2-(1,1-difluoroethyl)-6-ethylpyrimidin-4-yl)amino)-5-(5-(methylamino)pyrazin-2- yl)pyridin-2-yl)acetamide 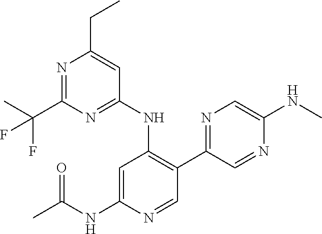 [SMILES: CCc1cc(Nc2cc(NC(C)=O)ncc2-c2cnc(NC)cn2)nc(C(C)(F)F)n1] LCMS m/z = 429.2 [M + H]+ |
| 861 | N-(4-((2-(1,1-difluoroethyl)-6-ethylpyrimidin-4-yl)amino)-5-(5-morpholinopyrazin-2- yl)pyridin-2-yl)acetamide 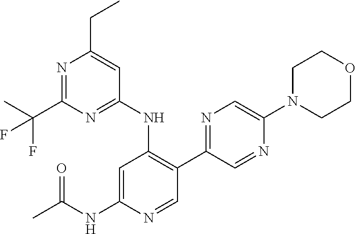 [SMILES: CCc1cc(Nc2cc(NC(C)=O)ncc2-c2cnc(N3CCOCC3)cn2)nc(C(C)(F)F)n1] LCMS m/z = 485.2 [M + H]+ |
| 862 | N-(4-((2-(1,1-difluoroethyl)-6-ethylpyrimidin-4-yl)amino)-5-(6-(dimethylamino)pyridazin- 3-yl)pyridin-2-yl)acetamide 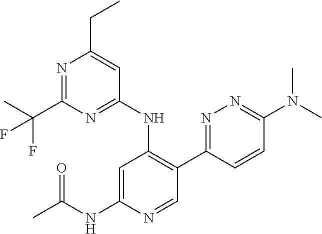 [SMILES: CCc1cc(Nc2cc(NC(C)=O)ncc2-c2ccc(N(C)C)nn2)nc(C(C)(F)F)n1] LCMS m/z = 443.2 [M + H]+ |
| 863 | N-(4-((2-(1,1-difluoroethyl)-6-ethylpyrimidin-4-yl)amino)-5-(6-morpholinopyridazin-3- yl)pyridin-2-yl)acetamide 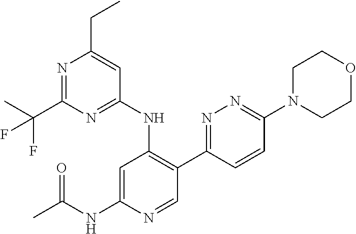 [SMILES: CCc1cc(Nc2cc(NC(C)=O)ncc2-c2ccc(N3CCOCC3)nn2)nc(C(C)(F)F)n1] LCMS m/z = 485.2 [M + H]+ |
| 864 | N-(5-(5-cyanopyrazin-2-yl)-4-((2-(1,1-difluoroethyl)-6-ethylpyrimidin-4-yl)amino)pyridin- 2-yl)acetamide 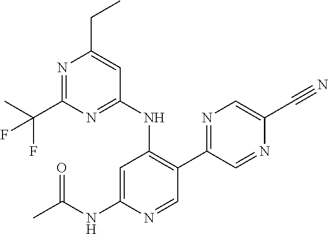 [SMILES: CCc1cc(Nc2cc(NC(C)=O)ncc2-c2cnc(C#N)cn2)nc(C(C)(F)F)n1] LCMS m/z = 425.1 [M + H]+ |
| 865 | N-(4-((2-(1,1-difluoroethyl)-6-ethylpyrimidin-4-yl)amino)-5-(1-methyl-6-oxo-1,6- dihydropyridazin-3-yl)pyridin-2-yl)acetamide 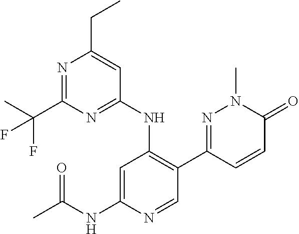 [SMILES: CCc1cc(Nc2cc(NC(C)=O)ncc2-c2ccc(=O)n(C)n2)nc(C(C)(F)F)n1] LCMS m/z = 430.2 [M + H]+ |
| 866 | N-(4-((2-(1,1-difluoroethyl)pyrimidin-4-yl)amino)-5-(5-((2- methoxyethyl)(methyl)amino)pyrazin-2-yl)pyridin-2-yl)acetamide 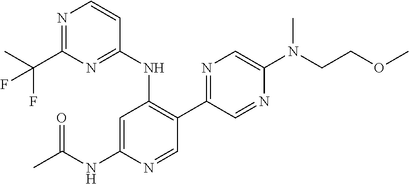 [SMILES: COCCN(C)c1cnc(-c2cnc(NC(C)=O)cc2Nc2ccnc(C(C)(F)F)n2)cn1] LCMS m/z = 459.2 [M + H]+ |
| 867 | N-(4-((2-(1,1-difluoroethyl)pyrimidin-4-yl)amino)-5-(5-(3-methoxypyrrolidin-1-yl)pyrazin- 2-yl)pyridin-2-yl)acetamide 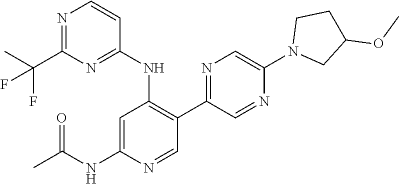 [SMILES: COC1CCN(c2cnc(-c3cnc(NC(C)=O)cc3Nc3ccnc(C(C)(F)F)n3)cn2)C1] LCMS m/z = 471.2 [M + H]+ |
| 868 | N-(4-((2-(1,1-difluoroethyl)pyrimidin-4-yl)amino)-5-(5-(3-methoxy-3-methylazetidin-1- yl)pyrazin-2-yl)pyridin-2-yl)acetamide 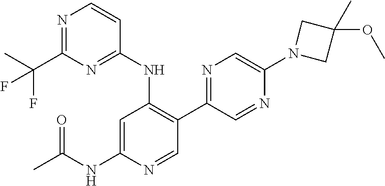 [SMILES: COC1(C)CN(c2cnc(-c3cnc(NC(C)=O)cc3Nc3ccnc(C(C)(F)F)n3)cn2)C1] LCMS m/z = 471.2 [M + H]+ |
| 869 | N-(5-(5-(2-oxa-6-azaspiro[3.4]octan-6-yl)pyrazin-2-yl)-4-((2-(1,1-difluoroethyl)pyrimidin-4- yl)amino)pyridin-2-yl)acetamide 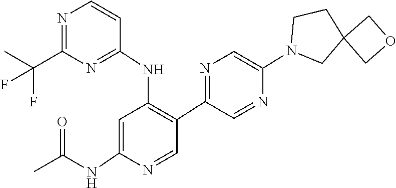 [SMILES: CC(=O)Nc1cc(Nc2ccnc(C(C)(F)F)n2)c(-c2cnc(N3CCC4(COC4)C3)cn2)cn1] LCMS m/z = 483.2 [M + H]+ |
| 870 | N-(5-(5-((1S,4S)-2-oxa-5-azabicyclo[2.2.1]heptan-5-yl)pyrazin-2-yl)-4-((2-(1,1- difluoroethyl)pyrimidin-4-yl)amino)pyridin-2-yl)acetamide 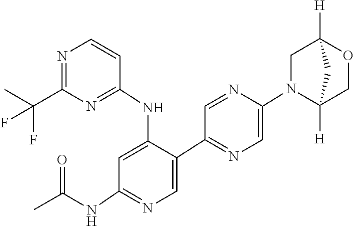 [SMILES: CC(=O)Nc1cc(Nc2ccnc(C(C)(F)F)n2)c(-c2cnc(N3C[C@@H]4C[C@H]3CO4)cn2)cn1] LCMS m/z = 469.2 [M + H]+ |
| 871 | N-(5-(5-((1R,4R)-2-oxa-5-azabicyclo[2.2.1]heptan-5-yl)pyrazin-2-yl)-4-((2-(1,1- difluoroethyl)pyrimidin-4-yl)amino)pyridin-2-yl)acetamide 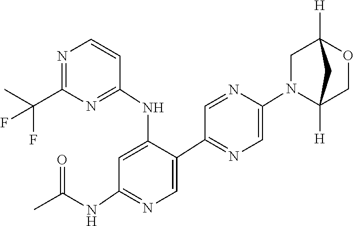 [SMILES: CC(=O)Nc1cc(Nc2ccnc(C(C)(F)F)n2)c(-c2cnc(N3C[C@H]4C[C@@H]3CO4)cn2)cn1] LCMS m/z = 469.2 [M + H]+ |
| 872 | N-(5-(5-(6-oxa-2-azaspiro[3.4]octan-2-yl)pyrazin-2-yl)-4-((2-(1,1-difluoroethyl)pyrimidin-4- yl)amino)pyridin-2-yl)acetamide 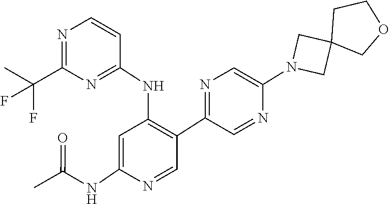 [SMILES: CC(=O)Nc1cc(Nc2ccnc(C(C)(F)F)n2)c(-c2cnc(N3CC4(CCOC4)C3)cn2)cn1] LCMS m/z = 483.2 [M + H]+ |
| 873 | rac-(R)-N-(4-((2-(1,1-difluoroethyl)pyrimidin-4-yl)amino)-5-(6-(3-methoxypyrrolidin-1- yl)pyridazin-3-yl)pyridin-2-yl)acetamide 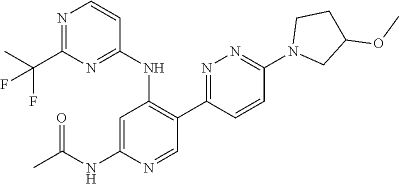 [SMILES: COC1CCN(c2ccc(-c3cnc(NC(C)=O)cc3Nc3ccnc(C(C)(F)F)n3)nn2)C1] LCMS m/z = 471.2 [M + H]+ |
| 874 | N-(4-((2-(1,1-difluoroethyl)pyrimidin-4-yl)amino)-5-(6-(3-methoxy-3-methylazetidin-1- yl)pyridazin-3-yl)pyridin-2-yl)acetamide 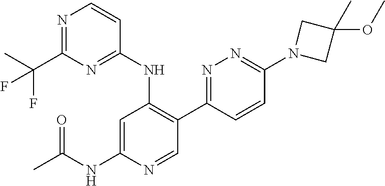 [SMILES: COC1(C)CN(c2ccc(-c3cnc(NC(C)=O)cc3Nc3ccnc(C(C)(F)F)n3)nn2)C1] LCMS m/z = 471.2 [M + H]+ |
| 875 | N-(5-(6-(2-oxa-6-azaspiro[3.3]heptan-6-yl)pyridazin-3-yl)-4-((2-(1,1- difluoroethyl)pyrimidin-4-yl)amino)pyridin-2-yl)acetamide 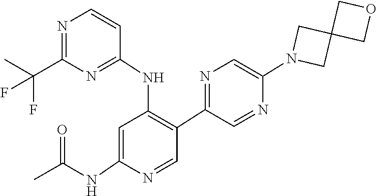 [SMILES: CC(=O)Nc1cc(Nc2ccnc(C(C)(F)F)n2)c(-c2cnc(N3CC4(COC4)C3)cn2)cn1] LCMS m/z = 469.2 [M + H]+ |
| 876 | N-(5-(6-((1S,4S)-2-oxa-5-azabicyclo[2.2.1]heptan-5-yl)pyridazin-3-yl)-4-((2-(1,1- difluoroethyl)pyrimidin-4-yl)amino)pyridin-2-yl)acetamide 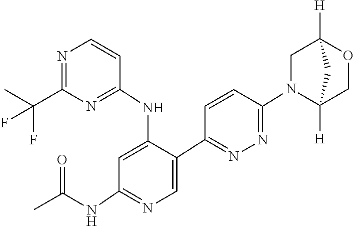 [SMILES: CC(=O)Nc1cc(Nc2ccnc(C(C)(F)F)n2)c(-c2ccc(N3C[C@@H]4C[C@H]3CO4)nn2)cn1] LCMS m/z = 469.2 [M + H]+ |
| 877 | N-(5-(6-(6-oxa-2-azaspiro[3.4]octan-2-yl)pyridazin-3-yl)-4-((2-(1,1-difluoroethyl)pyrimidin- 4-yl)amino)pyridin-2-yl)acetamide 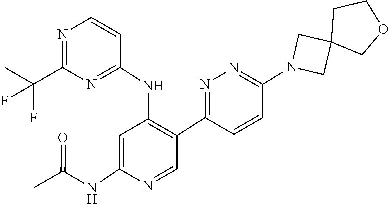 [SMILES: CC(=O)Nc1cc(Nc2ccnc(C(C)(F)F)n2)c(-c2ccc(N3CC4(CCOC4)C3)nn2)cn1] LCMS m/z = 483.2 [M + H]+ |
| 878 | N-(4′-((2-(1,1-difluoroethyl)pyrimidin-4-yl)amino)-5-(pyrrolidin-1-ylmethyl)-[2,3′- bipyridin]-6′-yl)acetamide 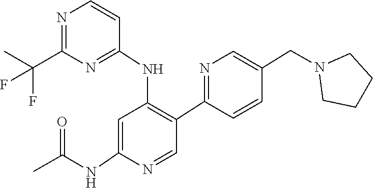 [SMILES: CC(=O)Nc1cc(Nc2ccnc(C(C)(F)F)n2)c(-c2ccc(CN3CCCC3)cn2)cn1] LCMS m/z = 454.2 [M + H]+ |
| 879 | N-(4′-((2-(1,1-difluoroethyl)pyrimidin-4-yl)amino)-5-(((2- methoxyethyl)(methyl)amino)methyl)-[2,3′-bipyridin]-6′-yl)acetamide 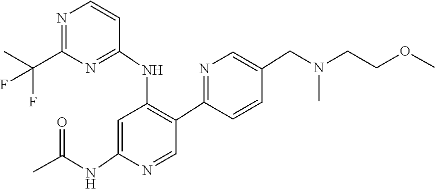 [SMILES: COCCN(C)Cc1ccc(-c2cnc(NC(C)=O)cc2Nc2ccnc(C(C)(F)F)n2)nc1] LCMS m/z = 472.2 [M + H]+ |
| 880 | (R)-N-(4′-((2-(1,1-difluoroethyl)pyrimidin-4-yl)amino)-5-((3-methylmorpholino)methyl)- [2,3′-bipyridin]-6′-yl)acetamide 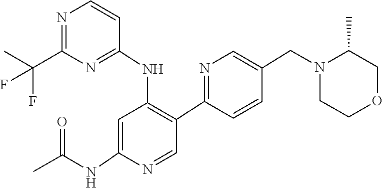 [SMILES: CC(=O)Nc1cc(Nc2ccnc(C(C)(F)F)n2)c(-c2ccc(CN3CCOC[C@H]3C)cn2)cn1] LCMS m/z = 484.2 [M + H]+ |
| 881 | N-(4′-((2-(1,1-difluoroethyl)pyrimidin-4-yl)amino)-5-((3-methoxypyrrolidin-1-yl)methyl)- [2,3′-bipyridin]-6′-yl)acetamide 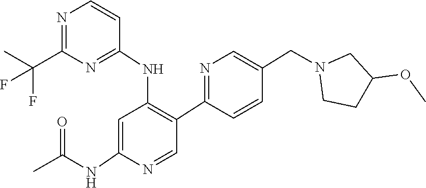 [SMILES: COC1CCN(Cc2ccc(-c3cnc(NC(C)=O)cc3Nc3ccnc(C(C)(F)F)n3)nc2)C1] LCMS m/z = 484.2 [M + H]+ |
| 882 | N-(4′-((2-(1,1-difluoroethyl)pyrimidin-4-yl)amino)-5-((3-methoxy-3-methylazetidin-1- yl)methyl)-[2,3′-bipyridin]-6′-yl)acetamide 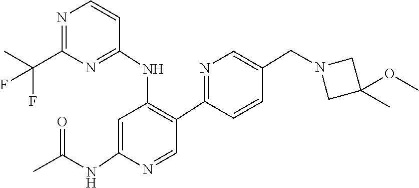 [SMILES: COC1(C)CN(Cc2ccc(-c3cnc(NC(C)=O)cc3Nc3ccnc(C(C)(F)F)n3)nc2)C1] LCMS m/z = 484.2 [M + H]+ |
| 883 | N-(5-((2-oxa-6-azaspiro[3.3]heptan-6-yl)methyl)-4′-((2-(1,1-difluoroethyl)pyrimidin-4- yl)amino)-[2,3′-bipyridin]-6′-yl)acetamide 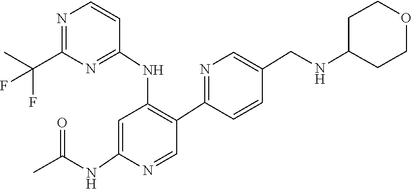 [SMILES: CC(=O)Nc1cc(Nc2ccnc(C(C)(F)F)n2)c(-c2ccc(CNC3CCOCC3)cn2)cn1] LCMS m/z = 484.2 [M + H]+ |
| 884 | rac-N-(5-(((1R,4R)-2-oxa-5-azabicyclo[2.2.1]heptan-5-yl)methyl)-4′-((2-(1,1- difluoroethyl)pyrimidin-4-yl)amino)-[2,3′-bipyridin]-6′-yl)acetamide 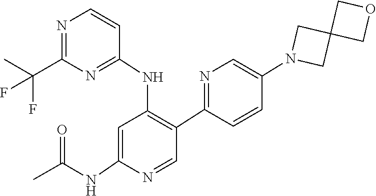 LCMS m/z = 482.2 [M + H]+ |
| 885 | N-(5-(((1S,4S)-2-oxa-5-azabicyclo[2.2.1]heptan-5-yl)methyl)-4′-((2-(1,1- difluoroethyl)pyrimidin-4-yl)amino)-[2,3′-bipyridin]-6′-yl)acetamide 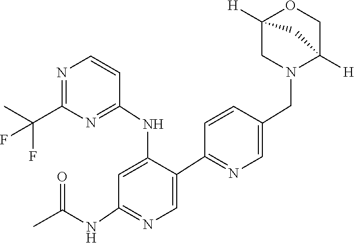 [SMILES: CC(=O)Nc1cc(Nc2ccnc(C(C)(F)F)n2)c(-c2ccc(CN3C[C@@H]4C[C@H]3CO4)cn2)cn1] LCMS m/z = 482.2 [M + H]+ |
| 886 | N-(5-(((1R,4R)-2-oxa-5-azabicyclo[2.2.1]heptan-5-yl)methyl)-4′-((2-(1,1- difluoroethyl)pyrimidin-4-yl)amino)-[2,3′-bipyridin]-6′-yl)acetamide 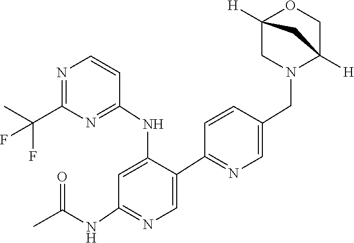 [SMILES: CC(=O)Nc1cc(Nc2ccnc(C(C)(F)F)n2)c(-c2ccc(CN3C[C@H]4C[C@@H]3CO4)cn2)cn1] LCMS m/z = 482.2 [M + H]+ |
| 887 | N-(5-(5-((1R,5S)-6-oxa-3-azabicyclo[3.1.1]heptan-3-yl)pyrazin-2-yl)-4-((2-(1,1- difluoroethyl)pyrimidin-4-yl)amino)pyridin-2-yl)acetamide 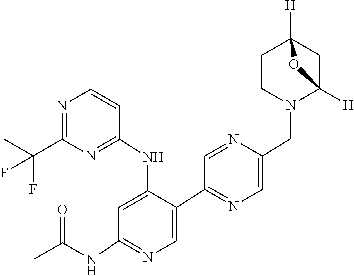 [SMILES: CC(=O)Nc1cc(Nc2ccnc(C(C)(F)F)n2)c(-c2cnc(CN3CC[C@@H]4C[C@H]3O4)cn2)cn1] LCMS m/z-469.2 [M + H]+ |
| 888 | N-(5-(5-(2-oxa-6-azaspiro[3.3]heptan-6-yl)pyrazin-2-yl)-4-((2-(1,1-difluoroethyl)pyrimidin- 4-yl)amino)pyridin-2-yl)acetamide 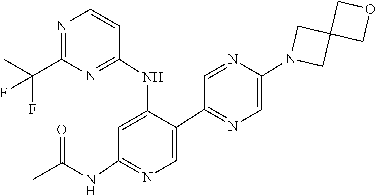 [SMILES: CC(=O)Nc1cc(Nc2ccnc(C(C)(F)F)n2)c(-c2cnc(N3CC4(COC4)C3)cn2)cn1] LCMS m/z = 469.2 [M + H]+ |
| 889 | N-(5-((6-oxa-2-azaspiro[3.4]octan-2-yl)methyl)-4′-((2-(1,1-difluoroethyl)pyrimidin-4- yl)amino)-[2,3′-bipyridin]-6′-yl)acetamide 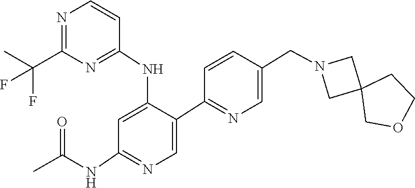 [SMILES: CC(=O)Nc1cc(Nc2ccnc(C(C)(F)F)n2)c(-c2ccc(CN3CC4(CCOC4)C3)cn2)cn1] LCMS m/z = 496.2 [M + H]+ |
| 890 | N-(5-(6-(2-oxa-6-azaspiro[3.4]octan-6-yl)pyridazin-3-yl)-4-((2-(1,1-difluoroethyl)pyrimidin- 4-yl)amino)pyridin-2-yl)acetamide 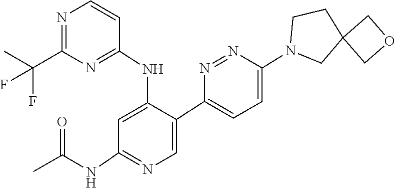 [SMILES: CC(=O)Nc1cc(Nc2ccnc(C(C)(F)F)n2)c(-c2ccc(N3CCC4(COC4)C3)nn2)cn1] LCMS m/z = 483.2 [M + H]+ |
| 891 | N-(4-((2-(1,1-difluoroethyl)-6-ethylpyrimidin-4-yl)amino)-5-(6-(difluoromethyl)pyrimidin- 4-yl)pyridin-2-yl)acetamide 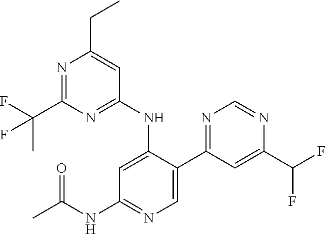 [SMILES: CCc1cc(Nc2cc(NC(C)=O)ncc2-c2cc(C(F)F)ncn2)nc(C(C)(F)F)n1] LCMS m/z = 450.2 [M + H]+ |
| 892 | (S)-N-(4′-((2-(1,1-difluoroethyl)pyrimidin-4-yl)amino)-5-((3-methylmorpholino)methyl)- [2,3′-bipyridin]-6′-yl)acetamide 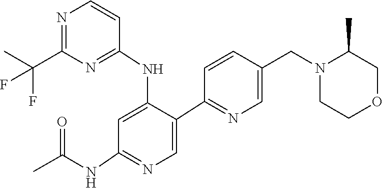 [SMILES: CC(=O)Nc1cc(Nc2ccnc(C(C)(F)F)n2)c(-c2ccc(CN3CCOC[C@@H]3C)cn2)cn1] LCMS m/z = 484.2 [M + H]+ |
| 893 | N-(4-((2-(1,1-difluoroethyl)-6-ethylpyrimidin-4-yl)amino)-5-(5-(pyrrolidin-1- ylmethyl)pyrazin-2-yl)pyridin-2-yl)acetamide 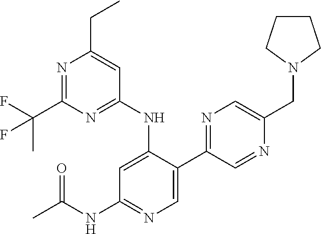 [SMILES: CCc1cc(Nc2cc(NC(C)=O)ncc2-c2cnc(CN3CCCC3)cn2)nc(C(C)(F)F)n1] LCMS m/z = 483.2 [M + H]+ |
| 894 | N-(5-(5-(((1S,4S)-2-oxa-5-azabicyclo[2.2.1]heptan-5-yl)methyl)pyrazin-2-yl)-4-((2-(1,1- difluoroethyl)-6-ethylpyrimidin-4-yl)amino)pyridin-2-yl)acetamide 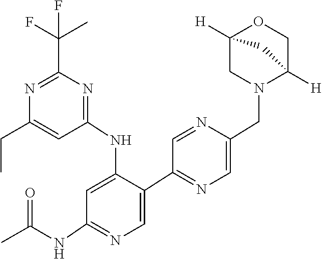 [SMILES: CCc1cc(Nc2cc(NC(C)=O)ncc2-c2cnc(CN3C[C@@H]4C[C@H]3CO4)cn2)nc(C(C)(F)F)n1] LCMS m/z = 511.2 [M + H]+ |
| 895 | N-(4-((2-(1,1-difluoroethyl)-6-ethylpyrimidin-4-yl)amino)-5-(5- ((dimethylamino)methyl)pyrazin-2-yl)pyridin-2-yl)acetamide 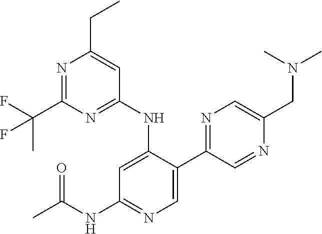 [SMILES: CCc1cc(Nc2cc(NC(C)=O)ncc2-c2cnc(CN(C)C)cn2)nc(C(C)(F)F)n1] LCMS m/z = 457.2 [M + H]+ |
| 896 | N-(4-((2-(1,1-difluoroethyl)pyrimidin-4-yl)amino)-5-(5-(2-methoxypropan-2-yl)pyrazin-2- yl)pyridin-2-yl)acetamide 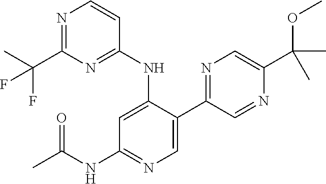 [SMILES: COC(C)(C)c1cnc(-c2cnc(NC(C)=O)cc2Nc2ccnc(C(C)(F)F)n2)cn1] LCMS m/z = 444.2 [M + H]+ |
| 897 | N-(5-(6-((1R,4R)-2-oxa-5-azabicyclo[2.2.1]heptan-5-yl)pyridazin-3-yl)-4-((2-(1,1- difluoroethyl)pyrimidin-4-yl)amino)pyridin-2-yl)acetamide 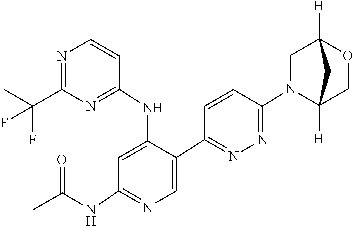 [SMILES: CC(=O)Nc1cc(Nc2ccnc(C(C)(F)F)n2)c(-c2ccc(N3C[C@H]4C[C@@H]3CO4)nn2)cn1] LCMS m/z = 469.2 [M + H]+ |
| 898 | N-(5-(5-(1,4-dioxan-2-yl)pyrazin-2-yl)-4-((2-(1,1-difluoroethyl)-6-ethylpyrimidin-4- yl)amino)pyridin-2-yl)acetamide 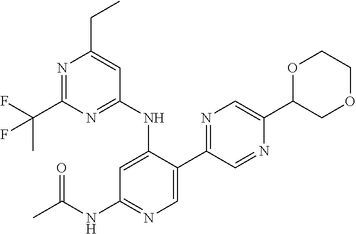 [SMILES: CCc1cc(Nc2cc(NC(C)=O)ncc2-c2cnc(C3COCCO3)cn2)nc(C(C)(F)F)n1] LCMS m/z = 486.2 [M + H]+ |
| 899 | N-(4-((2-(1,1-difluoroethyl)-6-ethylpyrimidin-4-yl)amino)-5-(5-(morpholinomethyl)pyrazin- 2-yl)pyridin-2-yl)acetamide 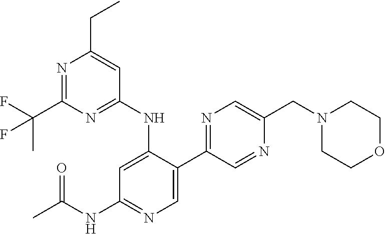 LCMS m/z = 511.2 [M + H]+ |
| 900 | N-(5-(5-(((1R,5S)-6-oxa-3-azabicyclo[3.1.1]heptan-3-yl)methyl)pyrazin-2-yl)-4-((2-(1,1- difluoroethyl)-6-ethylpyrimidin-4-yl)amino)pyridin-2-yl)acetamide 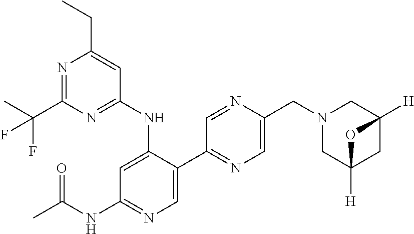 [SMILES: CCc1cc(Nc2cc(NC(C)=O)ncc2-c2cnc(CN3C[C@H]4C[C@@H](C3)O4)cn2)nc(C(C)(F)F)n1] LCMS m/z = 511.2 [M + H]+ |
| 901 | N-(4′-((2-(1,1-difluoroethyl)-6-ethylpyrimidin-4-yl)amino)-5-(pyrrolidin-1-ylmethyl)-[2,3′- bipyridin]-6′-yl)acetamide 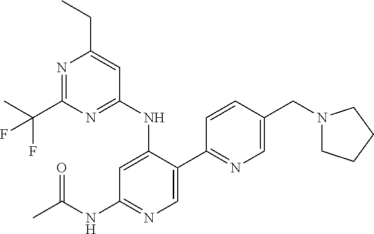 [SMILES: CCc1cc(Nc2cc(NC(C)=O)ncc2-c2ccc(CN3CCCC3)cn2)nc(C(C)(F)F)n1] LCMS m/z = 482.2 [M + H]+ |
| 902 | N-(5-(((1S,4S)-2-oxa-5-azabicyclo[2.2.1]heptan-5-yl)methyl)-4′-((2-(1,1-difluoroethyl)-6- ethylpyrimidin-4-yl)amino)-[2,3′-bipyridin]-6′-yl)acetamide 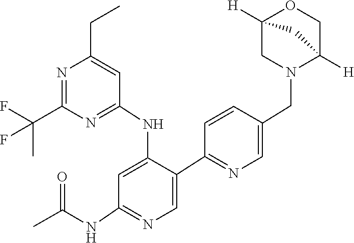 [SMILES: CCc1cc(Nc2cc(NC(C)=O)ncc2-c2ccc(CN3C[C@@H]4C[C@H]3CO4)cn2)nc(C(C)(F)F)n1] LCMS m/z = 510.2 [M + H]+ |
| 903 | N-(4-((2-(1,1-difluoroethyl)-6-ethylpyrimidin-4-yl)amino)-5-(5-(pyrrolidin-1- ylmethyl)pyrimidin-2-yl)pyridin-2-yl)acetamide 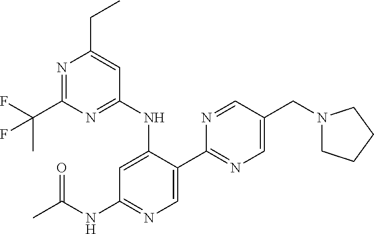 [SMILES: CCc1cc(Nc2cc(NC(C)=O)ncc2-c2ncc(CN3CCCC3)cn2)nc(C(C)(F)F)n1] LCMS m/z = 483.2 [M + H]+ |
| 904 | N-(4′-((2-(1,1-difluoroethyl)-6-ethylpyrimidin-4-yl)amino)-3-fluoro-5-(pyrrolidin-1- ylmethyl)-[2,3′-bipyridin]-6′-yl)acetamide 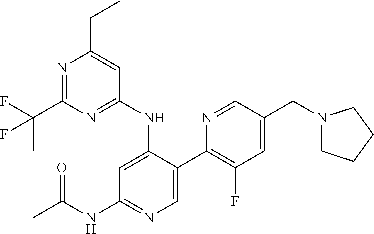 [SMILES: CCc1cc(Nc2cc(NC(C)=O)ncc2-c2ncc(CN3CCCC3)cc2F)nc(C(C)(F)F)n1] LCMS m/z = 500.2 [M + H]+ |
| 905 | N-(5-(6-(((1S,4S)-2-oxa-5-azabicyclo[2.2.1]heptan-5-yl)methyl)pyrimidin-4-yl)-4-((2-(1,1- difluoroethyl)-6-ethylpyrimidin-4-yl)amino)pyridin-2-yl)acetamide 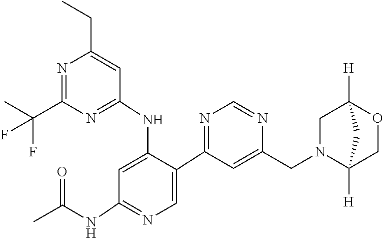 [SMILES: CCc1cc(Nc2cc(NC(C)=O)ncc2-c2cc(CN3C[C@@H]4C[C@H]3CO4)ncn2)nc(C(C)(F)F)n1] LCMS m/z = 511.2 [M + H]+ |
| 906 | N-(6-(((1S,4S)-2-oxa-5-azabicyclo[2.2.1]heptan-5-yl)methyl)-4′-((2-(1,1-difluoroethyl)-6- ethylpyrimidin-4-yl)amino)-5-fluoro-[2,3′-bipyridin]-6′-yl)acetamide 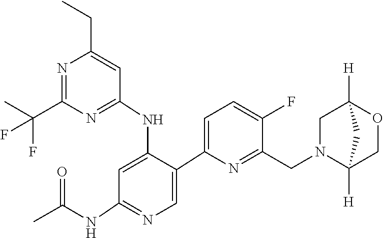 [SMILES: CCc1cc(Nc2cc(NC(C)=O)ncc2-c2ccc(F)c(CN3C[C@@H]4C[C@H]3CO4)n2)nc(C(C)(F)F)n1] LCMS m/z = 528.2 [M + H]+ |
| 907 | N-(5-(4-(((1R,4R)-2-oxa-5-azabicyclo[2.2.1]heptan-5-yl)methyl)pyrimidin-2-yl)-4-((2-(1,1- difluoroethyl)-6-ethylpyrimidin-4-yl)amino)pyridin-2-yl)acetamide 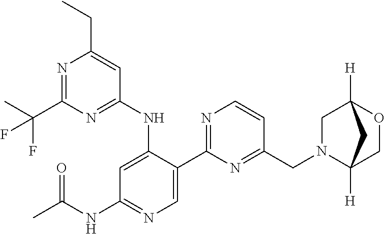 [SMILES: CCc1cc(Nc2cc(NC(C)=O)ncc2-c2nccc(CN3C[C@H]4C[C@@H]3CO4)n2)nc(C(C)(F)F)n1] LCMS m/z = 511.2 [M + H]+ |
| 908 | N-(6-(((1R,4R)-2-oxa-5-azabicyclo[2.2.1]heptan-5-yl)methyl)-4′-((2-(1,1-difluoroethyl)-6- ethylpyrimidin-4-yl)amino)-5-fluoro-[2,3′-bipyridin]-6′-yl)acetamide 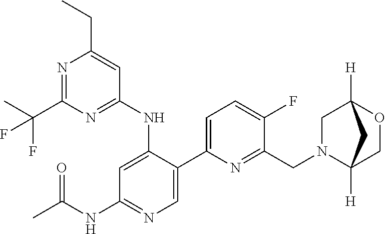 [SMILES: CCc1cc(Nc2cc(NC(C)=O)ncc2-c2ccc(F)c(CN3C[C@H]4C[C@@H]3CO4)n2)nc(C(C)(F)F)n1] LCMS m/z = 528.2 [M + H]+ |
| 909 | N-(4-((2-(1,1-difluoroethyl)-6-ethylpyrimidin-4-yl)amino)-5-(2- (morpholinomethyl)pyrimidin-4-yl)pyridin-2-yl)acetamide 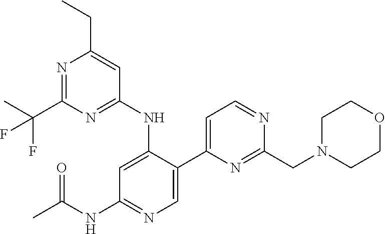 [SMILES: CCc1cc(Nc2cc(NC(C)=O)ncc2-c2ccnc(CN3CCOCC3)n2)nc(C(C)(F)F)n1] LCMS m/z = 499.2 [M + H]+ |
| 910 | N-(4′-((2-(1,1-difluoroethyl)-6-ethylpyrimidin-4-yl)amino)-5-fluoro-6-(morpholinomethyl)- [2,3′-bipyridin]-6′-yl)acetamide 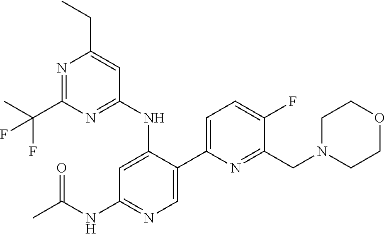 LCMS m/z = 499.2 [M + H]+ |
| 911 | N-(4′-((2-(1,1-difluoroethyl)-6-ethylpyrimidin-4-yl)amino)-6-((dimethylamino)methyl)-5- fluoro-[2,3′-bipyridin]-6′-yl)acetamide 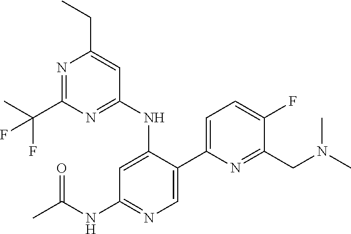 [SMILES: CCc1cc(Nc2cc(NC(C)=O)ncc2-c2ccc(F)c(CN(C)C)n2)nc(C(C)(F)F)n1] LCMS m/z = 474.2 [M + H]+ |
| 912 | N-(5-(((1R,5S)-6-oxa-3-azabicyclo[3.1.1]heptan-3-yl)methyl)-4′-((2-(1,1-difluoroethyl)-6- ethylpyrimidin-4-yl)amino)-[2,3′-bipyridin]-6′-yl)acetamide 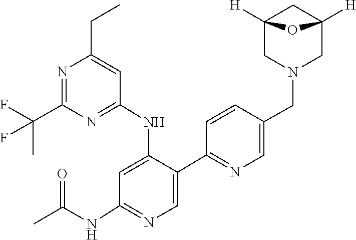 [SMILES: CCc1cc(Nc2cc(NC(C)=O)ncc2-c2ccc(CN3C[C@H]4C[C@@H](C3)O4)cn2)nc(C(C)(F)F)n1] LCMS m/z = 510 [M + H]+ |
| 913 | N-(5-(2-(((1R,5S)-6-oxa-3-azabicyclo[3.1.1]heptan-3-yl)methyl)pyrimidin-4-yl)-4-((2-(1,1- difluoroethyl)-6-ethylpyrimidin-4-yl)amino)pyridin-2-yl)acetamide 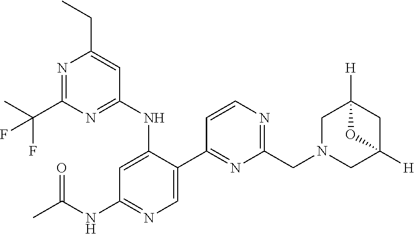 [SMILES: CCc1cc(Nc2cc(NC(C)=O)ncc2-c2ccnc(CN3C[C@H]4C[C@@H](C3)O4)n2)nc(C(C)(F)F)n1] LCMS m/z = 511.2 [M + H]+ |
| 914 | N-(5-(2-(((1R,5S)-6-oxa-3-azabicyclo[3.1.1]heptan-3-yl)methyl)pyrimidin-4-yl)-4-((2-(1,1- difluoroethyl)-6-ethylpyrimidin-4-yl)amino)pyridin-2-yl)acetamide 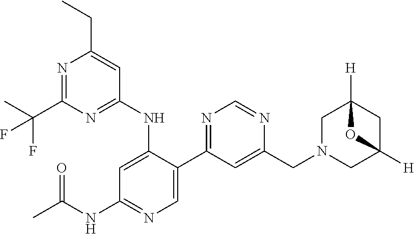 [SMILES: CCc1cc(Nc2cc(NC(C)=O)ncc2-c2cc(CN3C[C@H]4C[C@@H](C3)O4)ncn2)nc(C(C)(F)F)n1] LCMS m/z = 511.2 [M + H]+ |
| 915 | N-(6-(((1R,5S)-6-oxa-3-azabicyclo[3.1.1]heptan-3-yl)methyl)-4′-((2-(1,1-difluoroethyl)-6- ethylpyrimidin-4-yl)amino)-5-fluoro-[2,3′-bipyridin]-6′-yl)acetamide 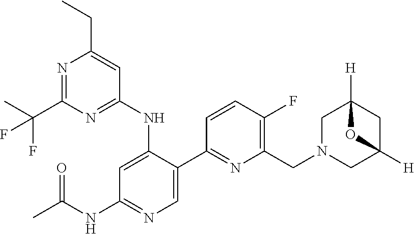 [SMILES: CCc1cc(Nc2cc(NC(C)=O)ncc2-c2ccc(F)c(CN3C[C@H]4C[C@@H](C3)O4)n2)nc(C(C)(F)F)n1] LCMS m/z = 528.2 [M + H]+ |
| 916 | N-(4′-((2-(1,1-difluoroethyl)-6-methylpyrimidin-4-yl)amino)-5-(dimethylphosphoryl)-[2,3′- bipyridin]-6′-yl)acetamide 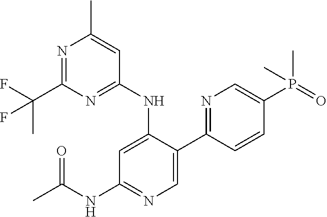 [SMILES: CC(=O)Nc1cc(Nc2cc(C)nc(C(C)(F)F)n2)c(-c2ccc(P(C)(C)=O)cn2)cn1] LCMS m/z = 460.2 1H NMR (500 MHz, DMSO-d6) δ (ppm) = 12.65 (s, 1H), 10.60 (s, 1H), 9.28 (s, 1H), 9.12 (dd, J = 1.5, 6.1 Hz, 1H), 8.86 (s, 1H), 8.32-8.27 (m, 1H), 8.24-8.21 (m, 1H), 7.15 (s, 1H), 2.43 (s, 3H), 2.14-2.05 (m, 6H), 1.78 (s, 3H), 1.75 (s, 3H). |
| 917 | N-(4-((2-(1,1-difluoroethyl)-6-methylpyrimidin-4-yl)amino)-5-(6- (dimethylphosphoryl)pyridazin-3-yl)pyridin-2-yl)acetamide 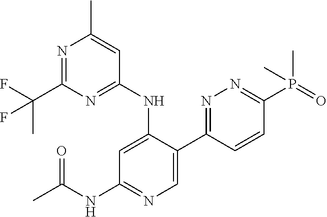 [SMILES: CC(=O)Nc1cc(Nc2cc(C)nc(C(C)(F)F)n2)c(-c2ccc(P(C)(C)=O)nn2)cn1] LCMS m/z = 462.2 1H NMR (500 MHz, DMSO-d6) δ (ppm) = 11.13 (s, 1H), 10.70 (s, 1H), 8.97 (s, 1H), 8.73 (s, 1H), 8.34 (dd, J = 2.7, 8.9 Hz, 1H), 8.19 (dd, J = 4.9, 8.9 Hz, 1H), 6.92 (s, 1H), 2.40 (s, 3H), 2.14 (s, 3H), 1.97 (t, J = 19.2 Hz, 3H), 1.84 (s, 3H), 1.82 (s, 3H). |
| 918 | N-(4-((2-(1,1-difluoroethyl)-6-ethylpyrimidin-4-yl)amino)-5-(2-methyl-2H-1,2,3-triazol-4- yl)pyridin-2-yl)acetamide 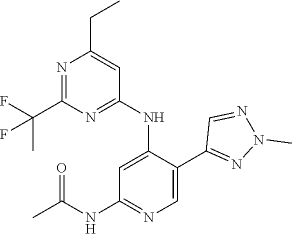 [SMILES: CCc1cc(Nc2cc(NC(C)=O)ncc2-c2cnn(C)n2)nc(C(C)(F)F)n1] LCMS m/z = 403.2 |
| 919 | 1-(4-((2-(1,1-difluoroethyl)-6-ethylpyrimidin-4-yl)amino)-5-(pyrazin-2-yl)pyridin-2-yl)-3- methylurea 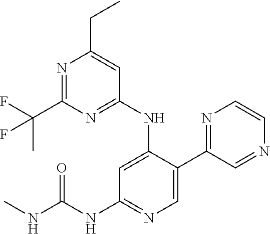 [SMILES: CCc1cc(Nc2cc(NC(=O)NC)ncc2-c2cnccn2)nc(C(C)(F)F)n1] LCMS m/z = 415.2 |
| 920 | 1-(4-((2-(1,1-difluoroethyl)-6-ethylpyrimidin-4-yl)amino)-5-(pyrazin-2-yl)pyridin-2-yl)-3- methylurea 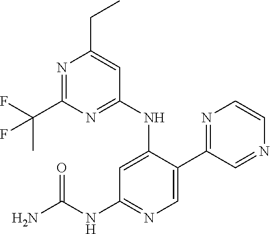 [SMILES: CCc1cc(Nc2cc(NC(N)=O)ncc2-c2cnccn2)nc(C(C)(F)F)n1] LCMS m/z = 401.2 |
| 921 | N-(4-((2-(1,1-difluoroethyl)-6-methylpyrimidin-4-yl)amino)-5-(5-(difluoromethyl)pyrazin-2- yl)pyridin-2-yl)acetamide 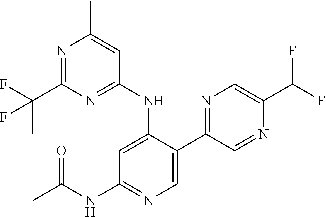 [SMILES: CC(=O)Nc1cc(Nc2cc(C)nc(C(C)(F)F)n2)c(-c2cnc(C(F)F)cn2)cn1] LCMS m/z = 436.1; ¹H NMR (500 MHz, CDCl₃) d 11.77 (s, 1H), 9.39 (s, 1H), 9.13 (s, 1H), 8.97 (s, 1H), 8.68 (s, 1H), 6.78 (t, 6.90-6.66 (m, 2H), 2.56 (s, 3H), 2.27 (s, 3H), 2.16 (t, J = 18.8 Hz, 3H). |
| 922 | N-(5-(2-(cyanomethyl)thiazol-4-yl)-4-((2-(1,1-difluoroethyl)pyrimidin-4-yl)amino)pyridin- 2-yl)acetamide 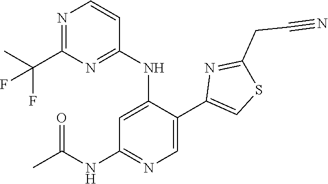 [SMILES: CC(=O)Nc1cc(Nc2ccnc(C(C)(F)F)n2)c(-c2csc(CC#N)n2)cn1] LCMS m/z = 416.1 [M + H]+ |

### Biological Assays

Compounds of the disclosure were assessed for their ability to inhibit TYK2, JAK1 and JAK2 activity. The inhibitory properties of the compounds of the disclosure described herein can be evidenced by testing in any one of the following protocols.

### JH2 Biochemical Assay

The inhibitory potency of compounds of the disclosure against the kinase activity of recombinantly generated JH2 domain of human Tyk2 was evaluated in a plate-based assay using a TR-FRET assay platform. Briefly, 2 nM of recombinant JH2 domain [10×His-tagged TYK2 JH2 domain (amino acid 575-876)] was combined with 2 nM probe ((S)-6-amino-9-(2-carboxy-4-((1-(3-(8-methyl-5-(methylamino)-8H-imidazo[4,5-d]thiazolo[5,4-b]pyridin-2-yl)phenyl)ethyl)carbamoyl)phenyl)-3-iminio-5-sulfo-3H-xanthene-4-sulfonate), 0.1 nM Tb-labeled anti-His antibody, and compounds of disclosure for 60 minutes. Compounds are tested at either 10 μM or 1 μM top concentration, 10 points of 3-fold dilution. The TR-FRET signal inversely correlates to the amount of probe displaced by compounds and signal was calculated by taking the ratio of fluorescence at 520 nm and 495 nm. The data was normalized and the percent activity versus log concentration of compound was fitted with a 4-parameter logistic model to generate IC₅₀ curves.

pSTAT4 Cell Assay

The inhibitory potency of compounds of the disclosure against the Tyk2 kinase activity on STAT4 was evaluated using an MSD-platform plate-based assay format. NK92 cells natively expressing STAT4 and Tyk2 were serum-starved to reduce background phosphorylation levels, then cells were treated compounds for 1 hr with a 10-point four-fold dilution series starting at 10 μM. Cells were then stimulated with 30 ng/mL IL2 for 15 minutes. Cells were lysed and pSTAT5 levels were quantitated using an MSD plate-based assay with anti-STAT4 antibodies. The data were normalized and the percent activity versus log concentration of compound were fitted with a 4-parameter logistic model to generate to generate IC₅₀ curves.

pSTAT5 Cell Assay

Compounds of the disclosure were assessed for their ability to inhibit the JAK2 kinase activity on STAT5 utilizing an MSD-platform plate-based assay format. TF1 cells natively expressing STAT5 and JAK2 were serum-starved to reduce background phosphorylation levels, then cells were treated with compounds of disclosure for 1 hour with a 10-point four-fold dilution series starting at 10 μM. Cells were then stimulated with 30 ng/mL IL-3 for 15 minutes. Cells were then lysed and pSTAT5 levels were quantitated using an MSD plate-based assay with anti-STAT5 antibodies. The data were normalized and the percent activity versus log concentration of compound was fitted with a 4-parameter logistic model to generate a curve and an IC₅₀ value.

pSTAT3 Cell Assay

The inhibitory potency of compounds of the disclosure against the JAK1 kinase activity on STAT3 was evaluated using an MSD-platform plate-based assay format. TF1 cells natively expressing STAT3 and JAK1 were serum-starved to reduce background phosphorylation levels, then cells were treated with compounds of the disclosure for 1 hour with a 10-point four-fold dilution series starting at 10 μM. Cells were then stimulated with 30 ng/mL interleukin 6 (IL-6) for 15 minutes. Cells were lysed and pSTAT3 levels were quantitated using an MSD plate-based assay with anti-STAT3 antibodies. The data were normalized and the percent activity versus log concentration of compound was fitted with a 4-parameter logistic model to generate IC₅₀ curves.

### Data for Examples

Table 1 shows the inhibitory activity of selected compounds of this disclosure to assess their ability to inhibit TYK2, JAK1 and JAK2, wherein each compound number corresponds to the compound numbering set forth in Examples 1-923 described herein. The measured IC₅₀ values were scored according to the following hierarchy:
“++++” represents: IC₅₀≤10 nM
“+++” represents: 10 nM<IC₅₀≤100 nM
“++” represents: 100 nM<IC₅₀<1000 nM
“+” represents: 1000 nM≤IC₅₀
NT means Not Tested

TABLE 1
Experimental Data

| | | TYK2; | JAK2; | JAK1; |
|---|---|---|---|---|
| Ex. | TYK2-JH2 | pSTAT4 | pSTAT5 | pSTAT3 |
| No. | IC₅₀ (nM) | IC₅₀ (nM) | IC₅₀ (nM) | IC₅₀ (nM) |
| 1 | ++++ | ++++ | + | + |
| 2 | +++ | + | NT | NT |
| 3 | ++++ | +++ | + | + |
| 4 | ++++ | +++ | + | + |
| 5 | ++++ | +++ | + | + |
| 6 | NT | + | NT | NT |
| 7 | NT | ++ | NT | NT |
| 8 | ++++ | ++ | NT | NT |
| 9 | ++++ | +++ | NT | NT |
| 10 | ++++ | +++ | + | + |
| 11 | ++++ | +++ | + | + |
| 12 | ++++ | +++ | + | + |
| 13 | ++++ | +++ | + | + |
| 14 | ++++ | +++ | + | + |
| 15 | ++++ | +++ | NT | NT |
| 16 | ++++ | ++++ | + | + |
| 17 | ++++ | +++ | + | + |
| 18 | ++++ | +++ | NT | NT |
| 19 | ++++ | +++ | + | + |
| 20 | ++++ | ++++ | + | + |
| 21 | ++++ | +++ | + | + |
| 22 | ++++ | ++++ | + | + |
| 23 | ++++ | ++ | NT | NT |
| 24 | ++++ | +++ | NT | NT |
| 25 | ++++ | +++ | NT | NT |
| 26 | ++++ | +++ | NT | NT |
| 27 | ++++ | ++ | NT | NT |
| 28 | +++ | NT | NT | NT |
| 29 | ++++ | ++ | + | + |
| 30 | ++++ | ++++ | NT | NT |
| 31 | ++++ | +++ | NT | NT |
| 32 | ++++ | +++ | + | + |
| 33 | ++ | NT | NT | NT |
| 34 | +++ | + | NT | NT |
| 35 | ++++ | ++ | NT | NT |
| 36 | ++++ | +++ | + | + |
| 37 | ++++ | ++ | + | + |
| 38 | ++++ | ++ | NT | NT |
| 39 | ++++ | +++ | + | + |
| 40 | ++++ | +++ | NT | NT |
| 41 | +++ | ++ | NT | NT |
| 42 | +++ | + | NT | NT |
| 43 | +++ | + | NT | NT |
| 44 | +++ | NT | NT | NT |
| 45 | ++++ | +++ | NT | NT |
| 46 | ++++ | +++ | + | + |
| 47 | +++ | + | NT | NT |
| 48 | ++ | + | NT | NT |
| 49 | ++++ | +++ | + | + |
| 50 | ++++ | ++++ | + | + |
| 51 | ++++ | +++ | NT | NT |
| 52 | ++++ | +++ | + | + |
| 53 | ++++ | +++ | NT | NT |
| 54 | + | NT | NT | NT |
| 55 | ++++ | ++++ | NT | NT |
| 56 | ++++ | ++++ | NT | NT |
| 57 | ++++ | +++ | NT | NT |
| 58 | ++++ | +++ | NT | NT |
| 59 | ++++ | ++ | NT | NT |
| 60 | ++++ | +++ | NT | NT |
| 61 | ++++ | +++ | NT | NT |
| 62 | ++++ | +++ | NT | NT |
| 63 | ++++ | ++++ | NT | NT |
| 64 | ++++ | +++ | NT | NT |
| 65 | ++++ | +++ | NT | NT |
| 66 | ++++ | ++++ | + | + |
| 67 | ++++ | +++ | + | + |
| 68 | ++++ | ++++ | NT | NT |
| 69 | ++++ | +++ | + | + |
| 70 | ++++ | ++++ | NT | NT |
| 71 | ++++ | ++++ | NT | NT |
| 72 | ++++ | ++ | NT | NT |
| 73 | NT | +++ | NT | NT |
| 74 | ++++ | +++ | NT | NT |
| 75 | ++++ | ++ | NT | NT |
| 76 | ++++ | ++++ | + | + |
| 77 | ++++ | ++++ | + | + |
| 78 | ++++ | +++ | NT | NT |
| 79 | ++++ | +++ | NT | NT |
| 80 | +++ | ++ | NT | NT |
| 81 | ++++ | +++ | NT | NT |
| 82 | ++++ | +++ | NT | NT |
| 83 | ++++ | ++ | NT | NT |
| 84 | ++++ | +++ | NT | NT |
| 85 | ++++ | +++ | NT | NT |
| 86 | ++++ | +++ | NT | NT |
| 87 | ++++ | ++++ | + | + |
| 88 | ++++ | ++++ | + | + |
| 89 | +++ | +++ | NT | NT |
| 90 | ++++ | ++ | NT | NT |
| 91 | ++++ | +++ | + | + |
| 92 | ++++ | +++ | NT | NT |
| 93 | ++++ | ++ | NT | NT |
| 94 | ++++ | +++ | NT | NT |
| 95 | +++ | ++ | NT | NT |
| 96 | ++++ | +++ | NT | NT |
| 97 | +++ | +++ | NT | NT |
| 98 | + | + | NT | NT |
| 99 | ++++ | ++++ | NT | NT |
| 100 | NT | ++++ | NT | NT |
| 101 | ++++ | +++ | + | + |
| 102 | NT | +++ | NT | NT |
| 103 | NT | ++ | NT | NT |
| 104 | NT | +++ | NT | NT |
| 105 | NT | ++++ | NT | NT |
| 106 | NT | +++ | NT | NT |
| 107 | NT | +++ | NT | NT |
| 108 | NT | +++ | NT | NT |
| 109 | NT | +++ | NT | NT |
| 110 | NT | +++ | NT | NT |
| 111 | NT | ++ | NT | NT |
| 112 | NT | +++ | NT | NT |
| 113 | NT | ++++ | NT | NT |
| 114 | NT | +++ | NT | NT |
| 115 | NT | ++++ | NT | NT |
| 116 | NT | +++ | NT | NT |
| 117 | NT | ++++ | NT | NT |
| 118 | NT | +++ | NT | NT |
| 119 | NT | +++ | + | + |
| 120 | NT | +++ | NT | NT |
| 121 | NT | +++ | NT | NT |
| 122 | NT | ++++ | NT | NT |
| 123 | NT | +++ | NT | NT |
| 124 | NT | ++++ | NT | NT |
| 125 | ++++ | +++ | NT | NT |
| 126 | NT | +++ | NT | NT |
| 127 | NT | ++++ | NT | NT |
| 128 | NT | +++ | NT | NT |
| 129 | NT | +++ | NT | NT |
| 130 | NT | +++ | NT | NT |
| 131 | NT | +++ | NT | NT |
| 132 | NT | +++ | NT | NT |
| 133 | NT | ++++ | NT | NT |
| 134 | NT | ++ | NT | NT |
| 135 | NT | ++++ | + | + |
| 136 | NT | ++++ | NT | NT |
| 137 | NT | +++ | NT | NT |
| 138 | ++ | + | NT | NT |
| 139 | ++++ | ++++ | + | + |
| 140 | ++++ | ++++ | NT | NT |
| 141 | NT | ++++ | NT | NT |
| 142 | ++++ | +++ | NT | NT |
| 143 | NT | + | NT | NT |
| 144 | ++++ | ++++ | NT | NT |
| 145 | ++++ | ++++ | + | + |
| 146 | ++++ | ++++ | + | + |
| 147 | ++++ | ++++ | NT | NT |
| 148 | NT | ++++ | NT | NT |
| 149 | NT | ++++ | NT | NT |
| 150 | ++++ | ++++ | NT | NT |
| 151 | ++ | NT | NT | NT |
| 152 | +++ | NT | NT | NT |
| 153 | ++++ | ++++ | NT | + |
| 154 | +++ | + | NT | NT |
| 155 | ++++ | +++ | NT | NT |
| 156 | ++++ | ++++ | NT | NT |
| 157 | ++++ | ++ | NT | NT |
| 158 | ++++ | ++++ | NT | NT |
| 159 | ++++ | +++ | NT | NT |
| 160 | ++++ | ++++ | NT | NT |
| 161 | ++++ | +++ | NT | NT |
| 162 | ++++ | ++++ | NT | NT |
| 163 | ++++ | +++ | NT | NT |
| 164 | ++++ | +++ | NT | NT |
| 165 | +++ | ++ | NT | NT |
| 166 | ++++ | +++ | NT | NT |
| 167 | ++++ | ++++ | NT | NT |
| 168 | ++++ | +++ | NT | NT |
| 169 | NT | NT | NT | NT |
| 170 | +++ | NT | NT | NT |
| 171 | ++++ | ++ | NT | NT |
| 172 | ++++ | ++++ | NT | NT |
| 173 | ++++ | ++++ | NT | NT |
| 174 | ++++ | ++++ | NT | NT |
| 175 | ++++ | ++++ | + | + |
| 176 | ++++ | ++++ | NT | NT |
| 177 | ++++ | ++++ | NT | NT |
| 178 | ++++ | ++++ | NT | NT |
| 179 | ++++ | ++++ | NT | NT |
| 180 | ++++ | ++++ | NT | NT |
| 181 | ++++ | ++++ | NT | NT |
| 182 | ++++ | +++ | + | + |
| 183 | ++++ | +++ | NT | NT |
| 184 | ++++ | +++ | NT | NT |
| 185 | ++++ | +++ | + | + |
| 186 | ++++ | +++ | NT | NT |
| 187 | +++ | + | NT | NT |
| 188 | ++++ | +++ | NT | NT |
| 189 | +++ | +++ | NT | NT |
| 190 | ++++ | +++ | NT | NT |
| 191 | ++++ | +++ | NT | NT |
| 192 | ++++ | +++ | NT | NT |
| 193 | ++++ | +++ | NT | NT |
| 194 | ++++ | ++++ | NT | NT |
| 195 | ++++ | +++ | + | + |
| 196 | ++++ | +++ | NT | NT |
| 197 | ++++ | ++++ | NT | NT |
| 198 | ++++ | +++ | NT | NT |
| 199 | +++ | NT | NT | NT |
| 200 | ++++ | +++ | + | + |
| 201 | ++++ | +++ | + | + |
| 202 | ++++ | ++++ | NT | NT |
| 203 | ++++ | ++++ | NT | NT |
| 204 | ++++ | ++++ | NT | NT |
| 205 | NT | + | NT | NT |
| 206 | ++++ | ++++ | NT | NT |
| 207 | NT | ++++ | NT | NT |
| 208 | ++++ | +++ | NT | NT |
| 209 | NT | ++++ | NT | NT |
| 210 | NT | ++++ | NT | NT |
| 211 | ++++ | ++++ | NT | NT |
| 212 | ++++ | ++++ | ++ | + |
| 213 | ++++ | ++++ | NT | NT |
| 214 | ++++ | ++++ | NT | + |
| 215 | ++++ | +++ | NT | NT |
| 216 | +++ | ++ | NT | NT |
| 217 | +++ | + | NT | NT |
| 218 | ++++ | ++++ | + | + |
| 219 | ++++ | ++++ | NT | NT |
| 220 | ++++ | NT | NT | NT |
| 221 | +++ | + | NT | NT |
| 222 | ++++ | ++++ | NT | NT |
| 223 | NT | +++ | NT | NT |
| 224 | NT | ++++ | NT | NT |
| 225 | ++++ | +++ | NT | NT |
| 226 | ++++ | ++++ | + | ++ |
| 227 | ++++ | ++++ | + | + |
| 228 | ++++ | ++++ | NT | NT |
| 229 | ++++ | ++++ | NT | NT |
| 230 | ++++ | ++++ | NT | NT |
| 231 | ++++ | ++++ | NT | NT |
| 232 | ++++ | ++++ | NT | NT |
| 233 | ++++ | ++++ | NT | NT |
| 234 | ++++ | ++++ | NT | NT |
| 235 | ++++ | ++++ | NT | NT |
| 236 | ++++ | ++++ | NT | NT |
| 237 | ++++ | ++++ | + | + |
| 238 | ++++ | ++++ | + | + |
| 239 | ++++ | ++++ | NT | NT |
| 240 | ++++ | ++++ | NT | NT |
| 241 | ++ | NT | NT | NT |
| 242 | ++++ | +++ | NT | NT |
| 243 | ++++ | +++ | NT | NT |
| 244 | ++++ | +++ | NT | NT |
| 245 | ++++ | +++ | NT | NT |
| 246 | ++++ | +++ | + | + |
| 247 | ++++ | + | NT | NT |
| 248 | +++ | NT | NT | NT |
| 249 | NT | +++ | NT | NT |
| 250 | ++++ | ++ | NT | NT |
| 251 | ++++ | ++++ | NT | NT |
| 252 | ++++ | ++++ | NT | NT |
| 253 | NT | + | NT | NT |
| 254 | ++++ | ++++ | NT | NT |
| 255 | ++++ | ++++ | NT | NT |
| 256 | +++ | + | NT | NT |
| 257 | ++++ | ++++ | NT | NT |
| 258 | ++ | ++ | NT | NT |
| 259 | ++++ | ++++ | NT | NT |
| 260 | ++++ | ++++ | + | + |
| 261 | ++++ | ++++ | NT | NT |
| 262 | ++++ | ++++ | NT | NT |
| 263 | ++++ | ++ | NT | NT |
| 264 | ++ | ++ | NT | NT |
| 265 | ++++ | ++++ | NT | NT |
| 266 | ++++ | ++++ | NT | ++ |
| 267 | ++++ | ++++ | NT | NT |
| 268 | ++++ | ++++ | + | + |
| 269 | ++++ | ++++ | NT | NT |
| 270 | ++++ | ++++ | + | + |
| 271 | ++++ | ++++ | NT | NT |
| 272 | ++++ | ++++ | NT | NT |
| 273 | +++ | ++ | NT | NT |
| 274 | ++++ | +++ | NT | NT |
| 275 | ++++ | ++ | NT | NT |
| 276 | ++++ | ++++ | + | + |
| 277 | ++++ | ++++ | NT | NT |
| 278 | +++ | + | NT | NT |
| 279 | +++ | ++ | NT | NT |
| 280 | ++++ | ++++ | NT | + |
| 281 | ++++ | +++ | NT | NT |
| 282 | ++++ | ++++ | NT | NT |
| 283 | ++++ | ++++ | NT | NT |
| 284 | NT | +++ | NT | NT |
| 285 | ++++ | NT | NT | NT |
| 286 | ++++ | ++ | NT | NT |
| 287 | ++++ | ++++ | NT | NT |
| 288 | ++++ | ++++ | NT | NT |
| 289 | ++++ | ++++ | + | + |
| 290 | NT | ++++ | NT | NT |
| 291 | NT | ++++ | + | ++ |
| 292 | NT | ++++ | + | + |
| 293 | ++++ | +++ | NT | NT |
| 294 | ++++ | ++++ | + | + |
| 295 | ++++ | +++ | NT | NT |
| 296 | +++ | ++ | NT | NT |
| 297 | ++++ | ++++ | NT | NT |
| 298 | ++++ | ++++ | + | + |
| 299 | ++++ | ++++ | NT | NT |
| 300 | ++++ | ++++ | + | + |
| 301 | +++ | ++ | NT | NT |
| 302 | ++++ | +++ | + | + |
| 303 | ++++ | +++ | NT | NT |
| 304 | ++++ | ++++ | + | + |
| 305 | ++++ | + | NT | NT |
| 306 | ++++ | ++++ | + | + |
| 307 | ++++ | +++ | NT | NT |
| 308 | ++++ | +++ | NT | NT |
| 309 | ++++ | +++ | NT | NT |
| 310 | ++++ | +++ | NT | NT |
| 311 | ++++ | +++ | + | + |
| 312 | ++++ | ++++ | NT | NT |
| 313 | ++++ | +++ | NT | NT |
| 314 | ++++ | ++++ | + | + |
| 315 | ++++ | +++ | + | + |
| 316 | ++++ | NT | NT | NT |
| 317 | ++++ | NT | NT | NT |
| 318 | ++++ | ++ | NT | NT |
| 319 | ++++ | +++ | NT | NT |
| 320 | ++++ | +++ | NT | NT |
| 321 | ++++ | +++ | NT | NT |
| 322 | ++++ | +++ | NT | NT |
| 323 | ++++ | ++++ | NT | NT |
| 324 | ++++ | +++ | NT | NT |
| 325 | ++++ | +++ | NT | NT |
| 326 | ++++ | +++ | NT | NT |
| 327 | ++++ | +++ | NT | NT |
| 328 | ++++ | ++++ | NT | NT |
| 329 | +++ | NT | NT | NT |
| 330 | +++ | + | NT | NT |
| 331 | +++ | ++ | NT | NT |
| 332 | ++++ | +++ | NT | NT |
| 333 | ++++ | +++ | NT | NT |
| 334 | +++ | ++ | NT | NT |
| 335 | ++++ | ++ | NT | NT |
| 336 | ++++ | +++ | + | + |
| 337 | ++++ | +++ | NT | NT |
| 338 | ++++ | +++ | NT | NT |
| 339 | ++++ | +++ | + | + |
| 340 | +++ | NT | NT | NT |
| 341 | ++++ | +++ | NT | NT |
| 342 | ++++ | ++ | NT | NT |
| 343 | ++++ | +++ | NT | NT |
| 344 | ++++ | +++ | NT | NT |
| 345 | +++ | ++ | NT | NT |
| 346 | ++++ | +++ | NT | NT |
| 347 | ++++ | NT | NT | NT |
| 348 | ++++ | +++ | NT | NT |
| 349 | ++++ | ++++ | NT | NT |
| 350 | ++++ | +++ | + | + |
| 351 | ++++ | ++++ | + | + |
| 352 | ++++ | +++ | NT | NT |
| 353 | ++++ | ++++ | + | + |
| 354 | ++++ | ++ | NT | NT |
| 355 | ++++ | +++ | NT | NT |
| 356 | + | + | NT | NT |
| 357 | +++ | + | NT | NT |
| 358 | NT | + | NT | NT |
| 359 | + | + | NT | NT |
| 360 | ++++ | +++ | + | + |
| 361 | ++++ | ++++ | NT | NT |
| 362 | ++++ | +++ | + | + |
| 363 | ++++ | ++++ | NT | NT |
| 364 | ++++ | +++ | + | + |
| 365 | ++++ | ++++ | NT | NT |
| 366 | ++++ | ++++ | NT | NT |
| 367 | ++++ | +++ | NT | NT |
| 368 | ++++ | ++++ | NT | NT |
| 369 | ++++ | +++ | NT | NT |
| 370 | ++++ | +++ | NT | NT |
| 371 | ++++ | ++++ | NT | NT |
| 372 | ++++ | +++ | NT | NT |
| 373 | ++++ | +++ | NT | NT |
| 374 | +++ | ++ | NT | NT |
| 375 | +++ | ++ | NT | NT |
| 376 | +++ | +++ | NT | NT |
| 377 | +++ | +++ | NT | NT |
| 378 | ++++ | +++ | NT | NT |
| 379 | ++++ | +++ | NT | NT |
| 380 | ++++ | +++ | NT | NT |
| 381 | ++++ | +++ | NT | NT |
| 382 | +++ | +++ | NT | NT |
| 383 | +++ | +++ | NT | NT |
| 384 | NT | NT | NT | NT |
| 385 | +++ | ++ | NT | NT |
| 386 | ++++ | +++ | NT | NT |
| 387 | NT | ++++ | NT | NT |
| 388 | ++++ | +++ | NT | NT |
| 389 | ++++ | +++ | NT | NT |
| 390 | +++ | ++ | NT | NT |
| 391 | ++++ | +++ | NT | NT |
| 392 | ++++ | ++++ | NT | NT |
| 393 | ++++ | +++ | NT | NT |
| 394 | ++++ | ++ | NT | NT |
| 395 | ++++ | ++ | NT | NT |
| 396 | NT | +++ | NT | NT |
| 397 | ++++ | NT | NT | NT |
| 398 | ++++ | +++ | NT | NT |
| 399 | ++++ | +++ | NT | NT |
| 400 | ++++ | NT | NT | NT |
| 401 | ++++ | ++ | NT | NT |
| 402 | +++ | ++ | NT | NT |
| 403 | ++++ | NT | NT | NT |
| 404 | ++++ | +++ | NT | NT |
| 405 | ++++ | +++ | NT | NT |
| 406 | ++++ | ++++ | + | + |
| 407 | ++++ | ++++ | NT | NT |
| 408 | ++++ | ++++ | NT | NT |
| 409 | ++++ | +++ | NT | NT |
| 410 | ++++ | ++++ | NT | NT |
| 411 | ++++ | +++ | NT | NT |
| 412 | ++++ | +++ | NT | NT |
| 413 | ++++ | ++++ | NT | NT |
| 414 | ++++ | ++++ | NT | NT |
| 415 | ++++ | ++++ | NT | NT |
| 416 | ++++ | ++ | NT | NT |
| 417 | ++++ | ++++ | NT | NT |
| 418 | ++++ | ++++ | NT | NT |
| 419 | ++++ | ++++ | NT | NT |
| 420 | ++++ | +++ | NT | NT |
| 421 | ++++ | +++ | NT | NT |
| 422 | ++++ | +++ | NT | NT |
| 423 | ++++ | +++ | NT | NT |
| 424 | ++++ | +++ | NT | NT |
| 425 | ++++ | ++++ | + | + |
| 426 | ++++ | ++++ | + | + |
| 427 | ++++ | +++ | NT | NT |
| 428 | ++++ | +++ | NT | NT |
| 429 | ++++ | +++ | NT | NT |
| 430 | ++++ | +++ | + | + |
| 431 | ++++ | ++ | NT | NT |
| 432 | ++++ | +++ | NT | NT |
| 433 | ++++ | ++++ | NT | NT |
| 434 | ++++ | +++ | NT | NT |
| 435 | ++++ | NT | NT | NT |
| 436 | ++ | + | NT | NT |
| 437 | +++ | NT | NT | NT |
| 438 | ++ | NT | NT | NT |
| 439 | ++++ | +++ | NT | NT |
| 440 | ++++ | +++ | NT | NT |
| 441 | ++++ | ++ | NT | NT |
| 442 | +++ | +++ | NT | NT |
| 443 | ++++ | +++ | NT | NT |
| 444 | ++++ | +++ | NT | NT |
| 445 | ++++ | ++++ | + | + |
| 446 | ++++ | ++++ | + | + |
| 447 | ++++ | +++ | NT | NT |
| 448 | +++ | ++ | NT | NT |
| 449 | ++ | + | NT | NT |
| 450 | +++ | + | NT | NT |
| 451 | +++ | + | NT | NT |
| 452 | +++ | NT | NT | NT |
| 453 | ++++ | ++++ | + | + |
| 454 | ++++ | ++++ | + | + |
| 455 | ++++ | ++++ | NT | NT |
| 456 | ++++ | +++ | NT | NT |
| 457 | ++++ | +++ | NT | NT |
| 458 | ++++ | ++++ | NT | NT |
| 459 | ++++ | +++ | NT | NT |
| 460 | ++++ | +++ | NT | NT |
| 461 | ++++ | +++ | NT | NT |
| 462 | ++++ | ++++ | NT | NT |
| 463 | ++++ | ++++ | NT | NT |
| 464 | ++++ | ++++ | NT | NT |
| 465 | ++++ | +++ | NT | NT |
| 466 | ++++ | +++ | NT | NT |
| 467 | ++++ | +++ | NT | NT |
| 468 | ++++ | ++ | NT | NT |
| 469 | ++++ | ++ | NT | NT |
| 470 | ++++ | ++ | NT | NT |
| 471 | +++ | ++ | NT | NT |
| 472 | +++ | ++ | NT | NT |
| 473 | ++++ | +++ | NT | NT |
| 474 | ++++ | +++ | NT | NT |
| 475 | ++++ | +++ | NT | NT |
| 476 | ++++ | +++ | NT | NT |
| 477 | ++++ | +++ | NT | NT |
| 478 | ++++ | ++ | NT | NT |
| 479 | ++++ | ++ | NT | NT |
| 480 | NT | +++ | NT | NT |
| 481 | ++++ | +++ | NT | NT |
| 482 | ++++ | +++ | NT | NT |
| 483 | ++++ | +++ | NT | NT |
| 484 | ++++ | +++ | NT | NT |
| 485 | ++++ | +++ | NT | NT |
| 486 | +++ | + | + | + |
| 487 | ++++ | ++++ | NT | NT |
| 488 | ++++ | ++++ | + | + |
| 489 | ++++ | ++++ | + | + |
| 490 | ++++ | ++++ | + | + |
| 491 | +++ | + | NT | NT |
| 492 | ++++ | +++ | NT | NT |
| 493 | ++++ | +++ | NT | NT |
| 494 | ++++ | +++ | NT | NT |
| 495 | ++++ | +++ | NT | NT |
| 496 | +++ | + | NT | NT |
| 497 | ++++ | +++ | NT | NT |
| 498 | ++++ | ++ | NT | NT |
| 499 | ++++ | +++ | NT | NT |
| 500 | ++++ | ++ | NT | NT |
| 501 | ++++ | ++ | NT | NT |
| 502 | ++++ | ++ | NT | NT |
| 503 | ++++ | +++ | NT | NT |
| 504 | ++++ | +++ | NT | NT |
| 505 | ++++ | +++ | NT | NT |
| 506 | ++++ | ++ | NT | NT |
| 507 | ++++ | ++++ | NT | NT |
| 508 | ++++ | +++ | NT | NT |
| 509 | ++++ | +++ | NT | NT |
| 510 | ++++ | ++ | NT | NT |
| 511 | +++ | ++ | NT | NT |
| 512 | ++ | ++ | NT | NT |
| 513 | ++++ | +++ | NT | NT |
| 514 | ++++ | ++++ | NT | NT |
| 515 | ++++ | +++ | + | + |
| 516 | ++++ | ++ | NT | NT |
| 517 | +++ | +++ | NT | NT |
| 518 | ++++ | ++ | NT | NT |
| 519 | ++ | ++ | NT | NT |
| 520 | ++++ | ++++ | NT | NT |
| 521 | NT | NT | NT | NT |
| 522 | ++++ | ++++ | + | + |
| 523 | ++++ | ++++ | NT | NT |
| 524 | ++++ | ++++ | NT | NT |
| 525 | ++++ | +++ | NT | NT |
| 526 | ++++ | +++ | NT | NT |
| 527 | ++++ | ++++ | NT | NT |
| 528 | ++++ | ++++ | + | + |
| 529 | ++++ | ++++ | NT | NT |
| 530 | ++++ | ++++ | NT | NT |
| 531 | ++++ | ++++ | NT | NT |
| 532 | ++++ | ++ | NT | NT |
| 533 | ++++ | ++++ | NT | NT |
| 534 | ++++ | ++++ | + | + |
| 535 | +++ | + | NT | NT |
| 536 | ++++ | ++++ | NT | NT |
| 537 | ++++ | +++ | NT | NT |
| 538 | ++++ | ++++ | NT | NT |
| 539 | ++++ | ++++ | NT | NT |
| 540 | ++++ | ++++ | NT | NT |
| 541 | ++++ | +++ | NT | NT |
| 542 | ++++ | ++++ | NT | NT |
| 543 | ++++ | ++++ | NT | NT |
| 544 | NT | ++++ | NT | NT |
| 545 | ++++ | ++++ | NT | NT |
| 546 | NT | ++++ | NT | NT |
| 547 | NT | ++++ | NT | NT |
| 548 | NT | ++++ | NT | NT |
| 549 | ++++ | ++ | NT | NT |
| 550 | ++++ | ++ | NT | NT |
| 551 | ++++ | +++ | NT | NT |
| 552 | ++++ | +++ | + | + |
| 553 | +++ | ++ | NT | NT |
| 554 | +++ | + | NT | NT |
| 555 | ++++ | +++ | NT | NT |
| 556 | ++++ | +++ | NT | NT |
| 557 | ++++ | +++ | NT | NT |
| 558 | ++++ | ++++ | NT | NT |
| 559 | ++++ | +++ | NT | NT |
| 560 | +++ | + | NT | NT |
| 561 | +++ | NT | NT | NT |
| 562 | ++++ | +++ | NT | NT |
| 563 | ++++ | +++ | NT | NT |
| 564 | ++++ | ++++ | NT | NT |
| 565 | ++++ | ++++ | + | + |
| 566 | ++++ | ++++ | + | + |
| 567 | ++++ | +++ | NT | NT |
| 568 | ++++ | ++++ | NT | NT |
| 569 | ++++ | ++++ | NT | NT |
| 570 | ++++ | +++ | NT | NT |
| 571 | NT | +++ | NT | NT |
| 572 | ++++ | ++++ | NT | NT |
| 573 | ++++ | ++++ | NT | NT |
| 574 | NT | ++++ | NT | NT |
| 575 | ++++ | ++++ | NT | NT |
| 576 | ++++ | ++ | NT | NT |
| 577 | ++++ | ++++ | NT | NT |
| 578 | ++++ | ++++ | NT | NT |
| 579 | ++ | + | NT | NT |
| 580 | ++++ | ++++ | + | + |
| 581 | ++++ | +++ | NT | NT |
| 582 | ++++ | ++++ | NT | NT |
| 583 | ++++ | ++++ | NT | NT |
| 584 | ++++ | ++++ | + | + |
| 585 | ++++ | ++++ | NT | NT |
| 586 | ++++ | +++ | NT | NT |
| 587 | ++++ | ++++ | NT | NT |
| 588 | ++++ | +++ | NT | NT |
| 589 | NT | ++++ | NT | NT |
| 590 | NT | +++ | NT | NT |
| 591 | ++++ | ++++ | NT | NT |
| 592 | ++++ | +++ | NT | NT |
| 593 | ++++ | ++++ | NT | NT |
| 594 | ++++ | ++++ | + | + |
| 595 | ++++ | +++ | NT | NT |
| 596 | ++++ | ++++ | + | + |
| 597 | ++++ | +++ | NT | NT |
| 598 | ++++ | +++ | + | + |
| 599 | ++++ | ++++ | NT | NT |
| 600 | +++ | ++ | NT | NT |
| 601 | ++++ | ++++ | NT | NT |
| 602 | ++++ | ++++ | + | + |
| 603 | ++++ | ++++ | NT | NT |
| 604 | +++ | + | NT | NT |
| 605 | ++++ | +++ | NT | NT |
| 606 | ++++ | ++++ | NT | NT |
| 607 | ++++ | ++++ | NT | NT |
| 608 | ++++ | ++++ | NT | + |
| 609 | NT | ++ | NT | NT |
| 610 | ++++ | ++++ | NT | NT |
| 611 | ++++ | ++++ | NT | NT |
| 612 | ++++ | ++++ | NT | NT |
| 613 | ++++ | ++++ | NT | NT |
| 614 | ++++ | ++++ | NT | NT |
| 615 | ++++ | ++++ | NT | NT |
| 616 | NT | +++ | NT | NT |
| 617 | ++++ | ++++ | NT | NT |
| 618 | ++++ | NT | NT | NT |
| 619 | ++++ | ++++ | NT | NT |
| 620 | ++++ | +++ | + | + |
| 621 | +++ | NT | NT | NT |
| 622 | ++++ | NT | NT | NT |
| 623 | ++++ | ++++ | NT | NT |
| 624 | ++++ | NT | NT | NT |
| 625 | NT | NT | NT | NT |
| 626 | NT | NT | NT | NT |
| 627 | ++ | + | NT | NT |
| 628 | ++++ | ++++ | NT | NT |
| 629 | NT | ++++ | NT | NT |
| 630 | ++++ | ++++ | NT | NT |
| 631 | ++++ | +++ | NT | NT |
| 632 | +++ | + | NT | NT |
| 633 | ++++ | ++++ | NT | NT |
| 634 | ++++ | ++++ | + | + |
| 635 | ++++ | ++++ | NT | NT |
| 636 | ++++ | ++++ | + | + |
| 637 | ++++ | ++++ | NT | NT |
| 638 | ++++ | ++++ | NT | NT |
| 639 | NT | ++++ | NT | NT |
| 640 | ++++ | ++++ | NT | NT |
| 641 | ++++ | ++++ | NT | NT |
| 642 | ++++ | ++++ | NT | + |
| 643 | ++++ | ++++ | NT | NT |
| 644 | ++++ | ++++ | NT | NT |
| 645 | NT | ++++ | NT | NT |
| 646 | NT | ++++ | NT | NT |
| 647 | ++++ | ++++ | NT | NT |
| 648 | ++++ | ++++ | + | + |
| 649 | ++++ | ++++ | NT | NT |
| 650 | ++++ | ++++ | NT | NT |
| 651 | NT | ++++ | NT | + |
| 652 | ++++ | ++++ | + | + |
| 653 | ++++ | +++ | NT | NT |
| 654 | ++++ | ++++ | NT | NT |
| 655 | ++++ | +++ | + | + |
| 656 | ++++ | ++++ | NT | NT |
| 657 | +++ | ++ | NT | NT |
| 658 | NT | + | NT | NT |
| 659 | NT | + | NT | NT |
| 660 | ++++ | ++++ | + | + |
| 661 | ++++ | ++++ | + | + |
| 662 | ++++ | +++ | NT | NT |
| 663 | +++ | + | NT | NT |
| 664 | NT | +++ | NT | NT |
| 665 | ++++ | +++ | NT | NT |
| 666 | ++++ | ++ | NT | NT |
| 667 | ++++ | +++ | NT | NT |
| 668 | ++++ | ++++ | + | + |
| 669 | ++++ | +++ | NT | NT |
| 670 | ++++ | +++ | NT | NT |
| 671 | ++++ | +++ | NT | NT |
| 672 | ++++ | ++++ | NT | NT |
| 673 | +++ | +++ | NT | NT |
| 674 | NT | + | NT | NT |
| 675 | ++++ | ++ | NT | NT |
| 676 | ++++ | ++++ | + | + |
| 677 | ++++ | ++++ | NT | NT |
| 678 | NT | +++ | NT | NT |
| 679 | NT | ++++ | NT | NT |
| 680 | ++++ | +++ | NT | NT |
| 681 | ++++ | ++++ | + | + |
| 682 | NT | ++++ | NT | NT |
| 683 | NT | ++++ | NT | NT |
| 684 | NT | ++++ | NT | NT |
| 685 | ++++ | +++ | NT | NT |
| 686 | +++ | + | NT | NT |
| 687 | ++++ | +++ | NT | NT |
| 688 | ++++ | ++ | NT | NT |
| 689 | +++ | + | NT | NT |
| 690 | +++ | +++ | NT | NT |
| 691 | +++ | ++ | NT | NT |
| 692 | ++++ | ++ | NT | NT |
| 693 | NT | +++ | NT | NT |
| 694 | ++++ | +++ | NT | NT |
| 695 | ++++ | ++ | NT | NT |
| 696 | ++++ | ++++ | NT | NT |
| 697 | ++++ | +++ | NT | NT |
| 698 | +++ | ++ | NT | NT |
| 699 | +++ | + | NT | NT |
| 700 | ++++ | +++ | NT | NT |
| 701 | ++++ | ++++ | NT | NT |
| 702 | ++++ | ++++ | NT | NT |
| 703 | ++++ | ++++ | NT | NT |
| 704 | ++++ | ++++ | NT | NT |
| 705 | ++++ | ++++ | NT | NT |
| 706 | ++++ | ++++ | NT | NT |
| 707 | ++++ | ++++ | + | + |
| 708 | ++++ | ++++ | + | + |
| 709 | ++++ | +++ | NT | NT |
| 710 | ++++ | ++++ | + | NT |
| 711 | ++++ | +++ | NT | NT |
| 712 | ++++ | ++++ | NT | NT |
| 713 | ++++ | NT | NT | NT |
| 714 | ++++ | ++++ | NT | NT |
| 715 | ++++ | ++++ | + | + |
| 716 | ++++ | +++ | NT | NT |
| 717 | ++++ | NT | NT | NT |
| 718 | ++++ | +++ | NT | NT |
| 719 | ++++ | ++++ | + | + |
| 720 | ++++ | ++++ | NT | NT |
| 721 | NT | ++++ | NT | NT |
| 722 | ++++ | ++++ | NT | NT |
| 723 | ++++ | +++ | NT | NT |
| 724 | +++ | + | + | + |
| 725 | ++ | NT | NT | NT |
| 726 | +++ | NT | NT | NT |
| 727 | ++ | NT | NT | NT |
| 728 | ++++ | +++ | NT | NT |
| 729 | ++++ | ++++ | NT | NT |
| 730 | +++ | ++ | NT | NT |
| 731 | ++++ | +++ | NT | NT |
| 732 | +++ | NT | NT | NT |
| 733 | ++++ | ++ | NT | NT |
| 734 | ++ | NT | NT | NT |
| 735 | +++ | NT | NT | NT |
| 736 | ++++ | ++++ | NT | NT |
| 737 | ++++ | ++++ | NT | NT |
| 738 | ++++ | ++++ | NT | NT |
| 739 | ++++ | NT | NT | NT |
| 740 | ++++ | ++++ | NT | NT |
| 741 | ++++ | ++++ | NT | NT |
| 742 | ++++ | NT | NT | NT |
| 743 | ++++ | +++ | NT | NT |
| 744 | ++++ | ++++ | NT | + |
| 745 | ++++ | +++ | NT | NT |
| 746 | ++++ | ++ | NT | + |
| 747 | ++++ | +++ | NT | + |
| 748 | ++++ | ++++ | NT | + |
| 749 | ++++ | +++ | NT | NT |
| 750 | ++++ | ++ | NT | NT |
| 751 | ++++ | +++ | NT | + |
| 752 | ++++ | NT | NT | NT |
| 753 | ++++ | + | NT | NT |
| 754 | ++++ | ++++ | NT | + |
| 755 | ++++ | ++++ | NT | NT |
| 756 | ++++ | ++ | NT | + |
| 757 | ++++ | ++++ | + | ++ |
| 758 | ++++ | ++++ | NT | NT |
| 759 | ++++ | +++ | NT | NT |
| 760 | ++++ | +++ | NT | NT |
| 761 | ++++ | + | NT | NT |
| 762 | ++++ | ++++ | NT | NT |
| 763 | ++++ | ++ | NT | NT |
| 764 | ++++ | +++ | NT | NT |
| 765 | ++++ | +++ | NT | NT |
| 766 | ++++ | ++ | NT | NT |
| 767 | ++++ | +++ | NT | NT |
| 768 | ++++ | ++++ | NT | + |
| 769 | ++++ | ++++ | NT | NT |
| 770 | ++++ | ++++ | NT | NT |
| 771 | ++++ | ++++ | NT | NT |
| 772 | ++++ | ++++ | NT | NT |
| 773 | ++++ | ++++ | NT | NT |
| 774 | ++++ | ++++ | NT | NT |
| 775 | ++++ | ++++ | NT | + |
| 776 | ++++ | ++++ | NT | + |
| 777 | +++ | + | NT | NT |
| 778 | ++++ | +++ | NT | NT |
| 779 | ++++ | +++ | NT | NT |
| 780 | ++++ | ++++ | NT | + |
| 781 | ++++ | ++++ | NT | NT |
| 782 | ++++ | ++++ | NT | NT |
| 783 | ++++ | ++++ | NT | NT |
| 784 | ++++ | ++++ | NT | NT |
| 785 | ++++ | NT | NT | NT |
| 786 | ++++ | ++++ | NT | NT |
| 787 | ++++ | ++++ | NT | NT |
| 788 | ++++ | ++++ | NT | NT |
| 789 | ++++ | ++++ | NT | NT |
| 790 | ++++ | ++++ | NT | NT |
| 791 | ++++ | ++++ | NT | NT |
| 792 | ++++ | +++ | NT | NT |
| 793 | ++++ | ++++ | NT | NT |
| 794 | ++++ | ++++ | NT | NT |
| 795 | ++++ | ++++ | NT | NT |
| 796 | ++++ | +++ | NT | NT |
| 797 | ++++ | ++++ | NT | NT |
| 798 | ++++ | ++++ | NT | + |
| 799 | ++++ | ++++ | + | + |
| 800 | ++++ | ++++ | NT | + |
| 801 | ++++ | ++++ | NT | NT |
| 802 | ++++ | ++++ | NT | NT |
| 803 | ++++ | ++++ | NT | NT |
| 804 | ++++ | ++++ | NT | NT |
| 805 | ++++ | +++ | NT | NT |
| 806 | ++++ | NT | NT | NT |
| 807 | ++++ | ++++ | NT | NT |
| 808 | +++ | + | NT | NT |
| 809 | +++ | NT | NT | NT |
| 810 | +++ | + | NT | NT |
| 811 | NT | +++ | NT | NT |
| 812 | +++ | + | NT | NT |
| 813 | ++++ | ++++ | NT | NT |
| 814 | ++++ | ++++ | NT | NT |
| 815 | ++++ | + | NT | NT |
| 816 | ++++ | ++ | NT | NT |
| 817 | +++ | + | NT | NT |
| 818 | ++++ | ++ | NT | NT |
| 819 | +++ | + | NT | NT |
| 820 | ++++ | NT | NT | NT |
| 821 | ++++ | ++++ | NT | NT |
| 822 | ++++ | ++++ | NT | NT |
| 823 | ++++ | +++ | NT | NT |
| 824 | ++++ | +++ | NT | NT |
| 825 | ++++ | +++ | + | + |
| 826 | ++++ | ++++ | + | + |
| 827 | ++++ | ++++ | NT | NT |
| 828 | ++++ | +++ | NT | NT |
| 829 | ++++ | +++ | NT | NT |
| 830 | ++++ | +++ | NT | NT |
| 831 | ++++ | ++++ | NT | NT |
| 832 | ++++ | ++++ | NT | NT |
| 833 | ++++ | ++++ | NT | NT |
| 834 | ++++ | ++++ | + | + |
| 835 | ++++ | ++++ | + | + |
| 836 | ++++ | ++++ | + | + |
| 837 | ++++ | ++++ | NT | NT |
| 838 | ++++ | +++ | NT | NT |
| 839 | ++++ | ++++ | NT | NT |
| 840 | ++++ | ++++ | + | + |
| 841 | ++++ | ++++ | NT | NT |
| 842 | ++++ | +++ | NT | NT |
| 843 | ++++ | +++ | NT | NT |
| 844 | ++++ | +++ | NT | NT |
| 845 | ++++ | ++++ | NT | NT |
| 846 | ++++ | ++++ | NT | NT |
| 847 | ++++ | +++ | NT | NT |
| 848 | ++++ | ++++ | NT | NT |
| 849 | ++++ | ++++ | NT | + |
| 850 | ++++ | ++ | NT | NT |
| 851 | ++++ | ++++ | + | + |
| 852 | ++++ | ++++ | NT | NT |
| 853 | ++++ | +++ | + | + |
| 854 | ++++ | +++ | NT | NT |
| 855 | ++++ | ++++ | NT | NT |
| 856 | ++++ | +++ | NT | NT |
| 857 | ++++ | ++++ | NT | + |
| 858 | ++++ | +++ | NT | NT |
| 859 | ++++ | ++++ | NT | + |
| 860 | ++ | + | NT | NT |
| 861 | ++++ | +++ | NT | + |
| 862 | ++++ | ++++ | NT | NT |
| 863 | ++++ | ++++ | NT | NT |
| 864 | ++++ | +++ | NT | NT |
| 865 | ++++ | +++ | NT | NT |
| 866 | ++++ | +++ | NT | NT |
| 867 | ++++ | +++ | NT | NT |
| 868 | ++++ | +++ | NT | NT |
| 869 | +++ | ++ | NT | NT |
| 870 | ++++ | +++ | NT | NT |
| 871 | ++++ | +++ | NT | NT |
| 872 | ++++ | +++ | NT | NT |
| 873 | ++++ | ++++ | NT | NT |
| 874 | ++++ | ++++ | NT | NT |
| 875 | ++++ | ++++ | NT | NT |
| 876 | ++++ | ++++ | NT | NT |
| 877 | ++++ | ++++ | NT | NT |
| 878 | ++++ | ++++ | NT | NT |
| 879 | ++++ | ++++ | NT | NT |
| 880 | ++++ | ++++ | NT | NT |
| 881 | ++++ | ++++ | NT | NT |
| 882 | ++++ | ++++ | NT | NT |
| 883 | ++++ | ++++ | NT | NT |
| 884 | ++++ | ++++ | NT | NT |
| 885 | ++++ | ++++ | NT | NT |
| 886 | ++++ | ++++ | NT | NT |
| 887 | ++++ | +++ | NT | NT |
| 888 | ++++ | ++ | NT | NT |
| 889 | ++++ | ++++ | NT | NT |
| 890 | ++++ | ++++ | NT | NT |
| 891 | ++++ | ++++ | NT | + |
| 892 | ++++ | ++++ | NT | + |
| 893 | ++++ | ++++ | NT | NT |
| 894 | ++++ | ++++ | NT | NT |
| 895 | ++++ | ++++ | NT | NT |
| 896 | ++++ | +++ | NT | NT |
| 897 | NT | NT | NT | NT |
| 898 | ++++ | ++++ | NT | NT |
| 899 | ++++ | +++ | NT | NT |
| 900 | ++++ | ++++ | NT | NT |
| 901 | NT | ++++ | NT | NT |
| 902 | NT | ++++ | NT | NT |
| 903 | NT | ++ | NT | NT |
| 904 | NT | ++++ | NT | NT |
| 905 | NT | +++ | NT | NT |
| 906 | NT | ++++ | NT | NT |
| 907 | NT | +++ | NT | NT |
| 908 | NT | ++++ | NT | NT |
| 909 | NT | ++ | NT | NT |
| 910 | NT | ++++ | NT | NT |
| 911 | NT | NT | NT | NT |
| 912 | NT | ++++ | NT | NT |
| 913 | NT | +++ | NT | NT |
| 914 | NT | + | NT | NT |
| 915 | NT | ++++ | NT | NT |
| 916 | NT | ++++ | NT | NT |
| 917 | NT | +++ | NT | NT |
| 918 | ++++ | ++++ | NT | + |
| 919 | ++++ | +++ | NT | NT |
| 920 | ++++ | ++++ | + | + |
| 921 | ++++ | +++ | NT | + |
| 922 | NT | ++++ | NT | NT |

What is claimed is:

1. A compound of Formula (I): 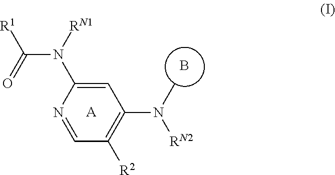
or a pharmaceutically acceptable salt thereof, wherein:
R¹ is H, C₁₋₆ alkyl, —OR^{1a}, —NR^{1b}R^{1c}, 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R^{1d};
R^{1a}, R^{1b}, and R^{1c} are each independently H, C₁₋₄alkyl, or 3 to 4 membered monocyclic carbocyclyl;
each R^{1d} is independently halo, oxo, —CN, —OR^{1a}, —NR^{1b}R^{1c}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
R² is selected from H, halo, C₁₋₆alkyl, C₃₋₇cycloalkyl, —OR^{2a}, —N(R^{2b})₂, 4- to 11-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the C₁₋₆alkyl, C₃₋₇cycloalkyl, 5- to 11-membered monocyclic or bicyclic heterocyclyl, and 5- to 6-membered monocyclic heteroaryl represented by R² are each optionally substituted by 1 to 3 R²⁰;
R^{2a} is selected from H, C₁₋₆alkyl, C₃₋₇cycloalkyl, 5- or 6-membered heteroaryl, and 4- to 7-membered monocyclic heterocyclyl having 1 to 4 heteroatoms independently selected from nitrogen, oxygen and sulfur, wherein the C₁₋₆alkyl, C₃₋₇cycloalkyl, 5- or 6-membered heteroaryl, and 4- to 7-membered monocyclic heterocyclyl represented by R^{2a} are optionally substituted with 1 to 3 R²⁰;
each R^{2b} is independently H, C₁₋₄alkyl, C₁₋₃alkyl-C₁₋₃alkoxy, C₁₋₄alkoxy, or 4- to 6-membered monocyclic heterocyclyl;
R²⁰, for each occurrence, is independently selected from halo, —CN, C₁₋₄alkyl, C₁₋₄ haloalkyl, —OR^{20b}, —C(O)R^{20b}, —C(O)N(R^{20b})₂, —N(R^{20b})₂, —SO₂R^{20b}, —P(O)(C₁₋₃alkyl)₂, phenyl, C₃₋₇cycloalkyl, 5- to 10-membered bicyclic carbocycle, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the C₁₋₄alkyl, phenyl, C₃₋₇cycloalkyl, 5- to 10-membered bicyclic carbocycle, 4- to 10-membered monocyclic or bicyclic heterocyclyl and 5- to 6-membered monocyclic heteroaryl represented by R²⁰ are each optionally substituted with 1 to 3 R²⁰⁰;
each R^{20b} is independently H, C₁₋₄alkyl or C₁₋₄alkoxy;
R^{20c} is H, C₁₋₄alkyl, C₁₋₄haloalkyl, C₃₋₆cycloalkyl, or 4- to 6-membered monocyclic heterocyclyl, wherein the C₁₋₄alkyl is optionally substituted by C₁₋₃alkoxy;
R²⁰⁰, for each occurrence, is independently selected from halo, —CN, C₁₋₄alkyl, C₁₋₃alkyl-C₁₋₃alkoxy, C₁₋₄haloalkyl, —OH, —N(R^{20b})₂, C₁₋₃alkoxy, C₁₋₃haloalkoxy, C₃₋₇cycloalkyl, and 5- to 10-membered monocyclic or bicyclic heterocyclyl optionally substituted with 1 to 3 C₁₋₃alkyl or C₁₋₃alkoxy;
Ring B is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{B};
each R^{B} is independently selected from halo, —CN, —OR^{Ba}, —N(R^{Bb})₂, —C(O)R^{Bc}, —C(O)OR^{Ba}, —SO₂R^{Bc}, C₁₋₆alkyl, C₂₋₆alkyenyl, phenyl, 3 to 7 membered monocyclic carbocyclyl, 4- to 7-membered monocyclic or bicyclic heterocyclyl having 1 to 4 heteroatoms independently selected from nitrogen, oxygen and sulfur, and 5 to 10 membered monocyclic or bicyclic heteroaryl having 1 to 3 heteroatoms independently selected from nitrogen, oxygen and sulfur, wherein the C₁₋₆alkyl, C₂₋₆alkyenyl, phenyl, 3 to 7 membered monocyclic carbocyclyl, 4- to 7-membered monocyclic heterocyclyl and 5 to 10 membered monocyclic or bicyclic heteroaryl represented by R^{B} are each optionally substituted by one or more R^{B1};
each R^{B1} is independently selected from halo, oxo, —CN, —OR^{Ba}, —N(R^{Bb})₂, C₁₋₄alkyl, C₁₋₄alkyl-R^{Bd}, C₁₋₄haloalkyl, —C(O)OR^{Ba}, phenyl, 5 to 6 membered heteroaryl, 3 to 7 membered monocyclic carbocyclyl, and 4 to 8 membered monocyclic heterocyclyl;
R^{Ba} is independently H, C₁₋₄alkyl, C₃₋₇cycloalkyl, or 4- to 8-membered monocyclic or bicycle heterocyclyl, wherein the C₁₋₄alkyl, C₃₋₇cycloalkyl and 4- to 8-membered monocyclic or bicycle heterocyclyl represented by R^{Ba} are each optionally substituted with 1 or 2 R^{B0};
each R^{B0} is independently halo, —CN, —OH, C₁₋₄alkyl or C₁₋₄alkoxy;
each R^{Bb} is independently H, C₁₋₄alkyl, C₁₋₄alkoxy or C₃₋₇cycloalkyl;
R^{Bc} is C₁₋₆alkyl or C₃₋₇cycloalkyl;
R^{Bd} is —C(O)OR^{Ba}, —N(R^{Bb})₂, —OR^{Ba}, 3 to 7 membered monocyclic carbocyclyl, or 4 to 8 membered monocyclic heterocyclyl; and
R^{N1} and R^{N2} are each independently H or C₁₋₄alkyl.

2. A compound of claim 1,
or a pharmaceutically acceptable salt thereof, wherein:
R¹ is H, C₁₋₆ alkyl, —OR^{1a}, —NR^{1b}R^{1c}, 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R^{1d};
R^{1a}, R^{1b}, and R^{1c} are each independently H, C₁₋₄alkyl, or 3 to 4 membered monocyclic carbocyclyl;
each R^{1d} is independently halo, oxo, —CN, —OR^{1a}, —NR^{1b}R^{1c}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
R² is selected from H, C₁₋₆alkyl, C₃₋₇cycloalkyl, —OR^{2a}, —N(R^{2b})₂, 4- to 11-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the C₁₋₆alkyl, C₃₋₇cycloalkyl, 5- to 11-membered monocyclic or bicyclic heterocyclyl, and 5- to 6-membered monocyclic heteroaryl represented by R² are each optionally substituted by 1 to 3 R²⁰;
R^{2a} is selected from H, C₁₋₆alkyl, C₃₋₇cycloalkyl and 4- to 7-membered monocyclic heterocyclyl having 1 to 4 heteroatoms independently selected from nitrogen, oxygen and sulfur, wherein the C₁₋₆alkyl, C₃₋₇cycloalkyl and 4- to 7-membered monocyclic heterocyclyl represented by R^{2a} are optionally substituted with 1 to 3 R²⁰;
each R^{2b} is independently H, C₁₋₄alkyl or C₁₋₄alkoxy;
R²⁰, for each occurrence, is independently selected from halo, —CN, C₁₋₄alkyl, C₁₋₄ haloalkyl, C₁₋₄alkoxy, —N(R^{20b})₂, phenyl, C₃₋₇cycloalkyl, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 3 heteroatoms independently selected from nitrogen, oxygen and sulfur, wherein the phenyl, C₃₋₇cycloalkyl, 4- to 10-membered monocyclic or bicyclic heterocyclyl and 5- to 6-membered monocyclic heteroaryl represented by R²⁰ are each optionally substituted with 1 to 3 R²⁰⁰;
each R^{20b} is independently H, C₁₋₄alkyl or C₁₋₄alkoxy;
R²⁰⁰, for each occurrence, is independently selected from halo, —CN, C₁₋₄alkyl, C₁₋₄haloalkyl, C₁₋₃alkoxy and C₃₋₇cycloalkyl;
Ring B is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{B};
each R^{B} is independently selected from halo, —CN, —OR^{Ba}, —N(R^{Bb})₂, —C(O)R^{Bc}, —C(O)OR^{Ba}, —SO₂R^{Bc}, C₁₋₆alkyl, phenyl, 3 to 7 membered monocyclic carbocyclyl 4- to 7-membered monocyclic heterocyclyl having 1 to 4 heteroatoms independently selected from nitrogen, oxygen and sulfur, and 5 to 10 membered monocyclic or bicyclic heteroaryl having 1 to 3 heteroatoms independently selected from nitrogen, oxygen and sulfur, wherein the C₁₋₆ alkyl, phenyl, 3 to 7 membered monocyclic carbocyclyl, 4- to 7-membered monocyclic heterocyclyl and 5 to 10 membered monocyclic or bicyclic heteroaryl represented by R^{B} are each optionally substituted by one or more R^{B1};
each R^{B1} is independently selected from halo, oxo, —CN, —OR^{Ba}, —N(R^{Bb})₂, C₁₋₄alkyl, C₁₋₄alkyl-R^{Bd}, C₁₋₄haloalkyl, —C(O)OR^{Ba}, phenyl, 5 to 6 membered heteroaryl, 3 to 7 membered monocyclic carbocyclyl, and 4 to 8 membered monocyclic heterocyclyl;
R^{Ba} is independently H, C₁₋₄alkyl, or C₃₋₇cycloalkyl, wherein the C₁₋₄alkyl and C₃₋₇cycloalkyl represented by R^{Ba} are each optionally substituted with 1 or 2 R^{B0};
each R^{B0} is independently halo, —CN, —OH, C₁₋₄alkyl or C₁₋₄alkoxy;
each R^{Bb} is independently H, C₁₋₄alkyl, C₁₋₄alkoxy or C₃₋₇cycloalkyl;
R^{Bc} is C₁₋₆alkyl or C₃₋₇cycloalkyl;
R^{Bd} is —C(O)OR^{Ba}, —N(R^{Bb})₂, —OR^{Ba}, 3 to 7 membered monocyclic carbocyclyl, or 4 to 8 membered monocyclic heterocyclyl; and
R^{N1} and R^{N2} are each independently H or C₁₋₄alkyl.

3. The compound of claim 1 or 2, or a pharmaceutically acceptable salt thereof, wherein Ring B is selected from phenyl, pyridinyl, pyrimidinyl and thiazolyl, each of which is substituted with one to three R^{B}.

4. The compound of any one of claims 1-3, or a pharmaceutically acceptable salt thereof, wherein R^{N1} and R^{N2} are each independently H or —CH₃.

5. The compound of any one of claims 1-4, wherein the compound is represented by Formula (II), (II′), or (III): 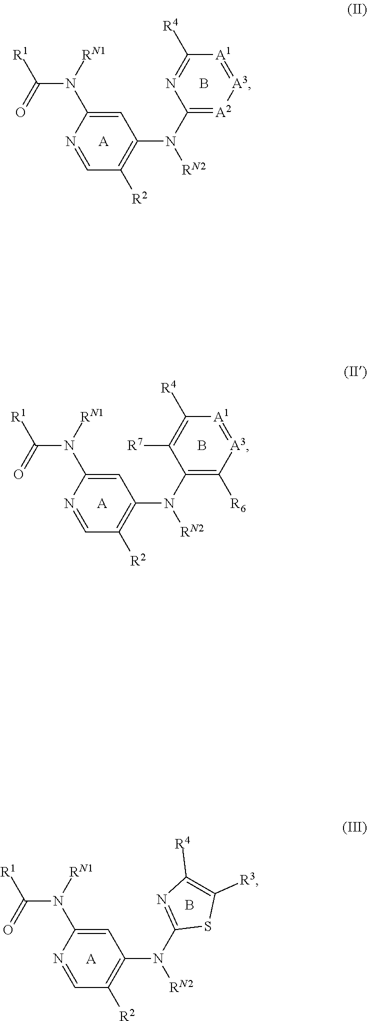
or a pharmaceutically acceptable salt thereof, wherein:
A¹ is N or CRS, A² is N or CR⁶, and A³ is N or CR³, provided no more than one of A¹, A², and A³ is N;
R³ is selected from H, halo, —OR^{3a}, —N(R^{3b})₂, C₁₋₆alkyl, C₁₋₄haloalkyl, C₁₋₃alkyl-C₁₋₃alkoxy, C2alkeneyl, C₃₋₇cycloalkyl, phenyl, and 5 to 10 membered monocyclic or bicyclic heteroaryl having 1 to 3 heteroatoms independently selected from nitrogen, oxygen and sulfur, wherein the phenyl and 5 to 10 membered monocyclic or bicyclic heteroaryl are each optionally substituted by 1 to 3 R^{3c};
R^{3a} is H, C₁₋₄alkyl, 4- to 8-membered monocyclic or bicycle heterocyclyl, or C₃₋₇cycloalkyl, each of which is optionally substituted with 1 or 2 R³⁰;
each R³¹ is independently halo, —CN, —OH, C₁₋₄alkyl or C₁₋₄alkoxy;
each R^{3b} is independently H, C₁₋₄alkyl, C₁₋₄alkoxy or C₃₋₇cycloalkyl;
each R³, is independently selected from halo, oxo, —CN, —OR^{3a}, —N(R^{3b})₂, C₁₋₄ alkyl, C₁₋₄alkyl-R^{3d}, C₁₋₄haloalkyl, —C(O)OR^{3a}, phenyl, 5 to 6 membered heteroaryl, 3 to 7 membered monocyclic carbocyclyl, and 4 to 8 membered monocyclic heterocyclyl;
R^{3d} is —C(O)OR^{3a}, —N(R^{3b})₂, —OR^{3a}, 3 to 7 membered monocyclic carbocyclyl,
or 4 to 8 membered monocyclic heterocyclyl;
R⁴ is selected from H, C₁₋₆alkyl, C₁₋₆haloalkyl, C₁₋₄alkoxy, C₁₋₃alkoxy-C₁₋₃alkoxy, C₁₋₃ haloalkoxy, —C₂haloalkenyl, —SO₂R^{4a}, 4- to 8-membered monocyclic or bicyclic heterocyclyl having 1 to 2 heteroatoms selected from nitrogen and oxygen, and C₃₋₇cycloalkyl, wherein the 4- to 8-membered monocyclic or bicyclic heterocyclyl and C₃₋₆cycloalkyl are each optionally substituted with 1 to 3 substituents independently selected from halo, C₁₋₄alkyl, C₁₋₃ haloalkyl, and C₁₋₃alkyl-C₁₋₃alkoxy;
R^{4a} is C₁₋₆alkyl;
R⁵ is H, halo, C₁₋₃alkyl, C₁₋₃haloalkyl or 5- to 6-membered heteroaryl having 1 to 3 heteroatoms independently selected from nitrogen, oxygen and sulfur, wherein the 5- to 6-membered heteroaryl represented by R⁵ is optionally substituted by 1 to 3 R⁵⁰; and
R⁵⁰, for each occurrence, is independently halo, C₁₋₄alkyl or C₁₋₄haloalkyl; and
R⁶ and R⁷ are each, independently, H, halo, C₁₋₃alkyl, C₁₋₃haloalkyl or C₁₋₄alkoxy.

6. The compound of any one of claims 1-4, wherein the compound is represented by Formula (II) or (III): 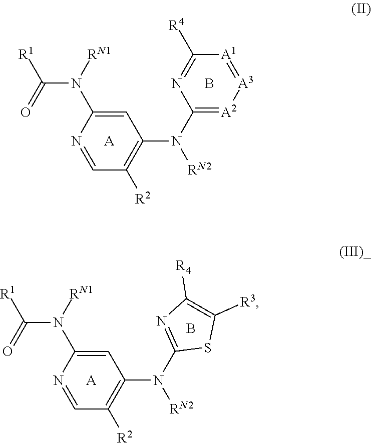
or a pharmaceutically acceptable salt thereof, wherein:
A¹ is N or CRS, A² is N or CR⁶, and A³ is N or CR³, provided no more than one of A¹, A², and A³ is N;
R³ is selected from H, —OR^{3a}, —N(R^{3b})₂, C₁₋₆alkyl, C₁₋₄haloalkyl, C₃₋₇cycloalkyl, phenyl, and 5 to 10 membered monocyclic or bicyclic heteroaryl having 1 to 3 heteroatoms independently selected from nitrogen, oxygen and sulfur, wherein the phenyl and 5 to 10 membered monocyclic or bicyclic heteroaryl are each optionally substituted by 1 to 3 R^{3c};
R^{3a} is H, C₁₋₄alkyl, or C₃₋₇cycloalkyl, each of which is optionally substituted with 1 or 2 R³⁰;
each R³⁰ is independently halo, —CN, —OH, C₁₋₄alkyl or C₁₋₄alkoxy;
each R^{3b} is independently H, C₁₋₄alkyl, C₁₋₄alkoxy or C₃₋₇cycloalkyl;
each R^{3c} is independently selected from halo, oxo, —CN, —OR^{3a}, —N(R^{3b})₂, C₁₋₄ alkyl, C₁₋₄alkyl-R^{3d}, C₁₋₄haloalkyl, —C(O)OR^{3a}, phenyl, 5 to 6 membered heteroaryl, 3 to 7 membered monocyclic carbocyclyl, and 4 to 8 membered monocyclic heterocyclyl;
R^{3d} is —C(O)OR^{3a}, —N(R^{3b})₂, —OR^{3a}, 3 to 7 membered monocyclic carbocyclyl,
or 4 to 8 membered monocyclic heterocyclyl;
R⁴ is selected from H, C₁₋₆alkyl, C₁₋₆haloalkyl, C₁₋₄alkoxy, —SO₂R^{4a}, 4- to 6-membered monocyclic heterocyclyl having 1 to 2 heteroatoms selected from nitrogen and oxygen and C₃₋₇cycloalkyl optionally substituted with 1 to 3 substituents independently halo or C₁₋₄alkyl;
R^{4a} is C₁₋₆alkyl;
R⁵ is H, halo, C₁₋₃alkyl, C₁₋₃haloalkyl or 5- to 6-membered heteroaryl having 1 to 3 heteroatoms independently selected from nitrogen, oxygen and sulfur, wherein the 5- to 6-membered heteroaryl represented by R⁵ is optionally substituted by 1 to 3 R⁵⁰; and
R⁵⁰, for each occurrence, is independently halo, C₁₋₄alkyl or C₁₋₄haloalkyl; and
R⁶ is H, halo, C₁₋₃alkyl, C₁₋₃haloalkyl or C₁₋₄alkoxy.

7. The compound of any one of claims 1-4, wherein the compound is represented by Formula (IV), (V), (VI), (VII), (VIII), or (IX): 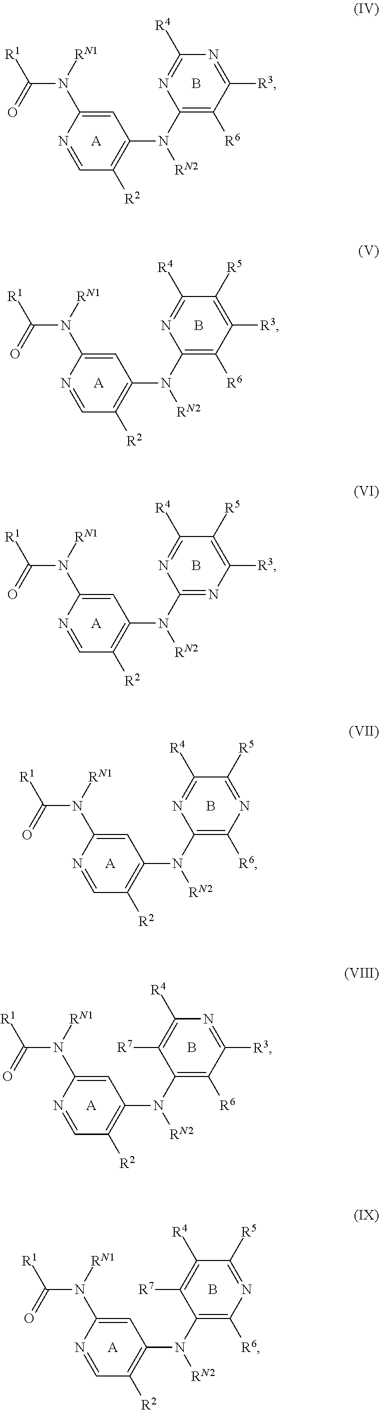
or a pharmaceutically acceptable salt thereof.

8. The compound of any one of claims 1-4, wherein the compound is represented by Formula (IV), (V), (VI), or (VII): 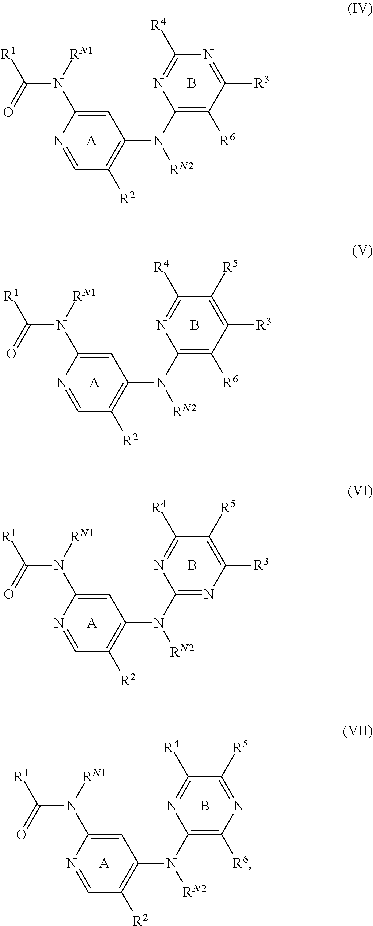
or a pharmaceutically acceptable salt thereof.

9. The compound of any one of claims 1-8, or a pharmaceutically acceptable salt thereof, wherein:
R¹ is H, C₁₋₄alkyl, —OR^{1a}, —NR^{1b}R^{1c}, or C₃₋₆cycloalkyl, wherein the C₁₋₄alkyl is optionally substituted with C₁₋₃alkoxy;
R^{1a} is C₁₋₃alkyl;
R^{1b} and R^{1c} are each, independently, H or C₁₋₃alkyl.

10. The compound of any one of claims 1-8, or a pharmaceutically acceptable salt thereof, wherein R¹ is C₁₋₄alkyl or C₃₋₆cycloalkyl, wherein the C₁₋₄alkyl is optionally substituted with C₁₋₃alkoxy.

11. The compound of any one of claims 1-8, or a pharmaceutically acceptable salt thereof, wherein R¹ is selected from H, —CH₃, —CH₂CH₃, —CH₂OCH₃, —CH₂CH₂OCH₃, —OCH₃, —NH₂, —NHCH₃ and cyclopropyl.

12. The compound of any one of claims 1-8, or a pharmaceutically acceptable salt thereof, wherein R¹ is selected from —CH₃, —CH₂CH₃, —CH₂OCH₃, —CH₂CH₂OCH₃ and cyclopropyl.

13. The compound of any one of claims 1-12, or a pharmaceutically acceptable salt thereof, wherein:
R² is selected from H, halo, C₁₋₄alkyl, —OR^{2a}, and —N(R^{2b})₂, wherein the C₁₋₄alkyl represented by R² is optionally substituted with 1 to 3 R²⁰;
R^{2a} is H, C₁₋₄alkyl, C₃₋₆cycloalkyl, 5- or 6-membered heteroaryl, or 4- to 6-membered monocyclic heterocyclyl having 1 to 4 heteroatoms independently selected from nitrogen, oxygen and sulfur, wherein the C₁₋₄alkyl, C₃₋₆cycloalkyl, 5- or 6-membered heteroaryl, and 4- to 6-membered monocyclic heterocyclyl represented by R^{2a} are each optionally substituted with 1 to 3 R²⁰;
R²⁰ is independently selected from halo, C₁₋₄alkyl, C₁₋₄alkoxy, —C(O)R^{20b}, —C(O)N(R^{20b})₂, —N(R^{20b})₂, phenyl, C₃₋₆cycloalkyl, 5- to 10-membered bicyclic carbocycle, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 4 heteroatoms independently selected from nitrogen, oxygen and sulfur, wherein the C₁₋₄alkyl, phenyl, C₃₋₆cycloalkyl, 4- to 10-membered monocyclic or bicyclic heterocyclyl and 5- to 6-membered heteroaryl are each optionally substituted with 1 to 3 R²⁰⁰;
R^{2b}, for each occurrence, is independently H or C₁₋₃alkyl;
R^{20b}, for each occurrence, is independently H or C₁₋₃alkyl; and
R²⁰⁰, for each occurrence, is independently selected from halo, C₁₋₄alkyl, C₁₋₃alkyl-C₁₋₃alkoxy, C₁₋₄haloalkyl, C₁₋₂alkoxy, and C₃₋₅cycloalkyl.

14. The compound of any one of claims 1-12, or a pharmaceutically acceptable salt thereof, wherein:
R² is selected from H, C₁₋₄alkyl, —OR^{2a}, and —N(R^{2b})₂, wherein the C₁₋₄alkyl represented by R² is optionally substituted with 1 to 3 R²⁰;
R^{2a} is H, C₁₋₄alkyl, C₃₋₆cycloalkyl or 4- to 6-membered monocyclic heterocyclyl having 1 to 4 heteroatoms independently selected from nitrogen, oxygen and sulfur, wherein the C₁₋₄alkyl, C₃₋₆cycloalkyl and 4- to 6-membered monocyclic heterocyclyl represented by R^{2a} are each optionally substituted with 1 to 3 R²⁰;
R²⁰ is independently selected from halo, C₁₋₃alkyl, C₁₋₃alkoxy, —N(R^{20b})₂, phenyl, C₃₋₆ cycloalkyl, 4- to 10-membered monocyclic or bicyclic heterocyclyl having 1 to 4 heteroatoms independently selected from nitrogen, oxygen and sulfur, and 5- to 6-membered monocyclic heteroaryl having 1 to 4 heteroatoms independently selected from nitrogen, oxygen and sulfur, wherein the phenyl, C₃₋₆cycloalkyl, 4- to 10-membered monocyclic or bicyclic heterocyclyl and 5- to 6-membered heteroaryl are each optionally substituted with 1 to 3 R²⁰⁰;
R^{2b}, for each occurrence, is independently H or C₁₋₃alkyl;
R^{20b}, for each occurrence, is independently H or C₁₋₃alkyl; and
R²⁰⁰, for each occurrence, is independently selected from halo, C₁₋₄alkyl, C₁₋₄haloalkyl, C₁₋₂alkoxy and C₃₋₅cycloalkyl.

15. The compound of claim 13 or 14, or a pharmaceutically acceptable salt thereof, wherein:
R² is H, C₁₋₄alkyl, —OR^{2a} or —N(R^{2b})₂, wherein the C₁₋₄alkyl represented by R² is optionally substituted with 1 to 3 substituents independently selected from halo, C₁₋₃alkoxy and —N(R^{20b})₂; and
R^{2a} is H or C₁₋₄alkyl optionally substituted with 1 to 3 substituents independently selected from halo, C₁₋₃alkoxy and —N(R^{20b})₂.

16. The compound of claim 13, or a pharmaceutically acceptable salt thereof, wherein:
R² is —OR²a;
R^{2a} is C₃₋₆cycloalkyl, 5- or 6-membered heteroaryl, or 4- to 6-membered monocyclic heterocyclyl having 1 to 2 heteroatoms independently selected from nitrogen and oxygen, wherein the C₃₋₆cycloalkyl, 5- or 6-membered heteroaryl, and 4- to 6-membered monocyclic heterocyclyl represented by R^{2a} are each optionally substituted with 1 to 3 substituents independently selected from halo, C₁₋₃alkyl and C₁₋₃alkoxy.

17. The compound of claim 14, or a pharmaceutically acceptable salt thereof, wherein:
R² is —OR²a;
R^{2a} is C₃₋₆cycloalkyl or 4- to 6-membered monocyclic heterocyclyl having 1 to 2 heteroatoms independently selected from nitrogen and oxygen, wherein the C₃₋₆cycloalkyl or 4- to 6-membered monocyclic heterocyclyl represented by R^{2a} are each optionally substituted with 1 to 3 substituents independently selected from halo, C₁₋₃alkyl and C₁₋₃alkoxy.

18. The compound of claim 16, or a pharmaceutically acceptable salt thereof, wherein R^{2a} is cyclopropyl, cyclobutyl, cyclopentyl, oxetanyl, azetidinyl, tetrahydrofuranyl pyrrolidinyl, pyrazinyl, pyridazinyl, or pyrazoyl, each of which is optionally substituted with 1 to 3 substituents independently selected from halo, C₁₋₃alkyl and C₁₋₃alkoxy.

19. The compound of claim 17, or a pharmaceutically acceptable salt thereof, wherein R^{2a} is cyclopropyl, cyclobutyl, cyclopentyl, oxetanyl, azetidinyl, tetrahydrofuranyl or pyrrolidinyl, each of which is optionally substituted with 1 to 3 substituents independently selected from halo, C₁₋₃alkyl and C₁₋₃alkoxy.

20. The compound of claim 16, or a pharmaceutically acceptable salt thereof, wherein R^{2a} is represented by the following: 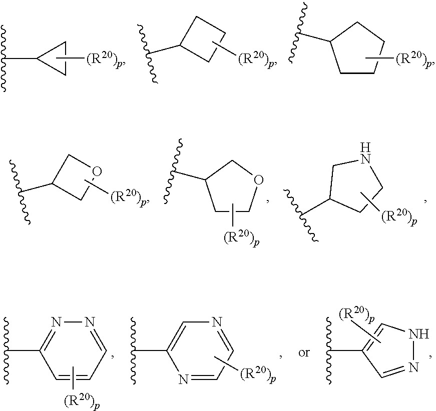
wherein p is 0, 1, 2 or 3; and each R²⁰ is independently halo, C₁₋₃alkyl and C₁₋₃alkoxy.

21. The compound of claim 17, or a pharmaceutically acceptable salt thereof, wherein R^{2a} is represented by the following: 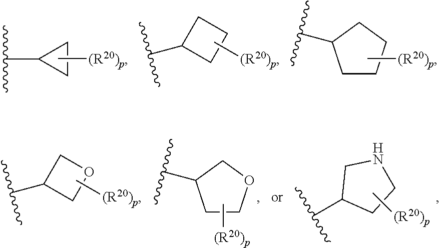
wherein p is 0, 1, 2 or 3; and each R²⁰ is independently halo, C₁₋₃alkyl and C₁₋₃alkoxy.

22. The compound of claim 20, or a pharmaceutically acceptable salt thereof, wherein R^{2a} is represented by the following:

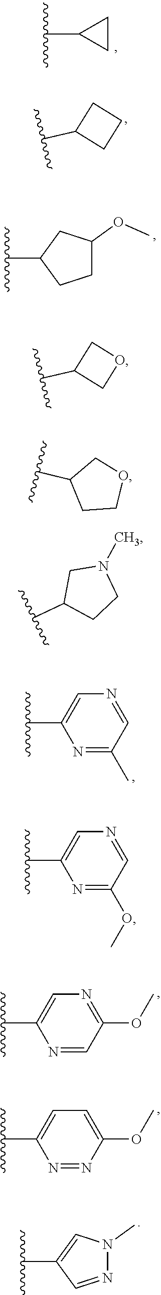

23. The compound of claim 21, or a pharmaceutically acceptable salt thereof, wherein R^{2a} is represented by the following:

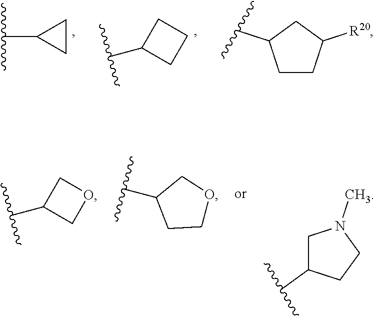

24. The compound of claim 13, or a pharmaceutically acceptable salt thereof, wherein:
R² is —OR^{2a} and R^{2a} is C₁₋₄alkyl substituted with one R²⁰; and
R²⁰ is phenyl, C₃₋₆cycloalkyl, 5- to 10-membered bicyclic carbocycle, 4- to 10-membered monocyclic or bicyclic heterocyclyl having 1 to 2 heteroatoms independently selected from nitrogen and oxygen, and 5- to 6-membered monocyclic heteroaryl having 1 to 3 heteroatoms independently selected from nitrogen, oxygen and sulfur, wherein the phenyl, C₃₋₆cycloalkyl, 5- to 10-membered bicyclic carbocycle, 4- to 10-membered monocyclic or bicyclic heterocyclyl and 5- to 6-membered monocyclic heteroaryl are each optionally substituted with 1 to 3 R²⁰⁰.

25. The compound of claim 14, or a pharmaceutically acceptable salt thereof, wherein:
R² is —OR^{2a} and R^{2a} is C₁₋₄alkyl substituted with one R²⁰; and
R²⁰ is phenyl, C₃₋₆cycloalkyl, 4- to 10-membered monocyclic or bicyclic heterocyclyl having 1 to 2 heteroatoms independently selected from nitrogen and oxygen, and 5- to 6-membered monocyclic heteroaryl having 1 to 3 heteroatoms independently selected from nitrogen, oxygen and sulfur, wherein the phenyl, C₃₋₆cycloalkyl, 4- to 10-membered monocyclic or bicyclic heterocyclyl and 5- to 6-membered monocyclic heteroaryl are each optionally substituted with 1 to 3 R²⁰⁰.

26. The compound of claim 24, or a pharmaceutically acceptable salt thereof, wherein R²⁰ is independently selected from azetindinyl, benzo[d][1,3]dioxolyl, cyclobutyl, cyclopropyl, spiro[2.2]pentanyl, bicyclo[1.1.1]pentanyl, 2-oxabicyclo[2.1.1]hexanyl, 5-oxaspiro[2.4]heptanyl, 6-oxaspiro[3.4]octanyl, dihydrofuranonyl, 1,3-dioxolanyl, morpholinyl, piperazinyl, 1,4-dioxanyl, 5,8-dioxaspiro[3.5]nonanyl, tetrahydropyranyl, 3-oxabicyclo[3.1.1]heptanyl, 2-oxabicyclo[2.2.2]octanyl, 7-oxabicyclo[2.2.1]heptanyl, imidazolyl, isoxazolyl, morpholinyl, oxabicyclo[2.2.1]hexanyl, oxadiazolyl, oxetanyl, oxazolyl, phenyl, furanyl, thiazoyl, isothiazolyl, thiadiazolyl, triazoyl, pyrazolyl, pyridinyl, pyrimidinyl, pyrazinyl, pyrrolidinyl and tetrahydrofuranyl, each of which is optionally substituted with 1 to 3 R²⁰⁰.

27. The compound of claim 25, or a pharmaceutically acceptable salt thereof, wherein R²⁰ is independently selected from azetindinyl, benzo[d][1,3]dioxolyl, cyclobutyl, cyclopropyl, dihydrofuranonyl, imidazolyl, isoxazolyl, morpholinyl, oxabicyclo[2.2.1]hexanyl, oxadiazolyl, oxetanyl, oxazolyl, phenyl, pyrazolyl, pyridinyl, pyrrolidinyl and tetrahydrofuranyl, each of which is optionally substituted with 1 to 3 R²⁰⁰.

28. The compound of claim 24, or a pharmaceutically acceptable salt thereof, wherein R²⁰ is independently selected from: 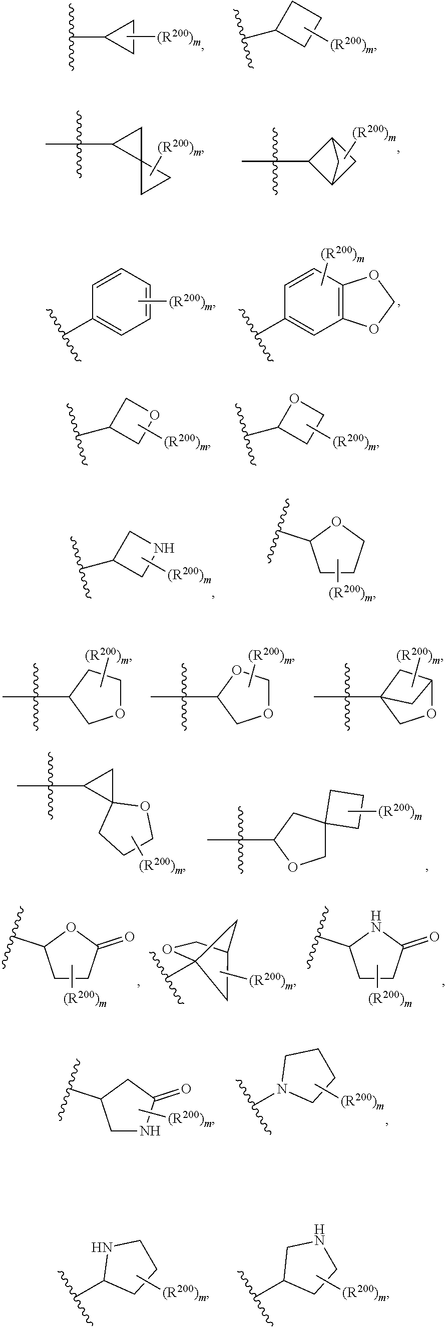 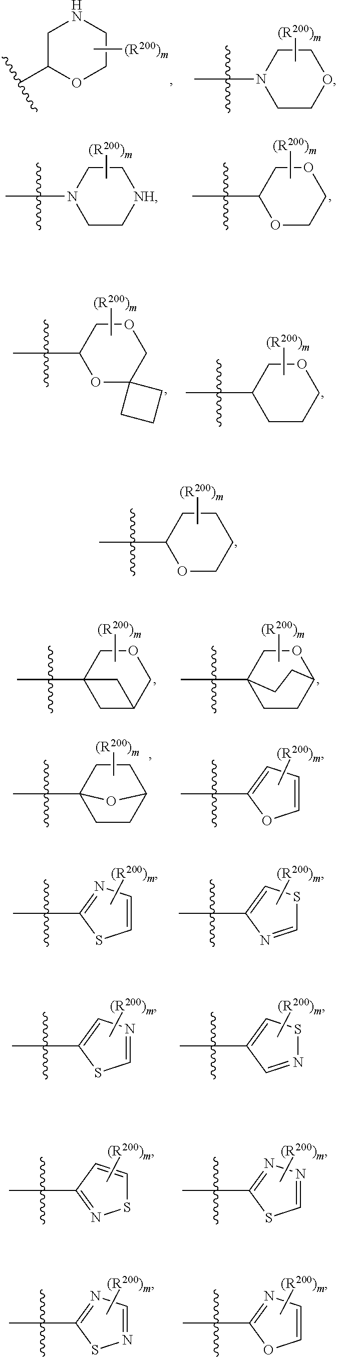 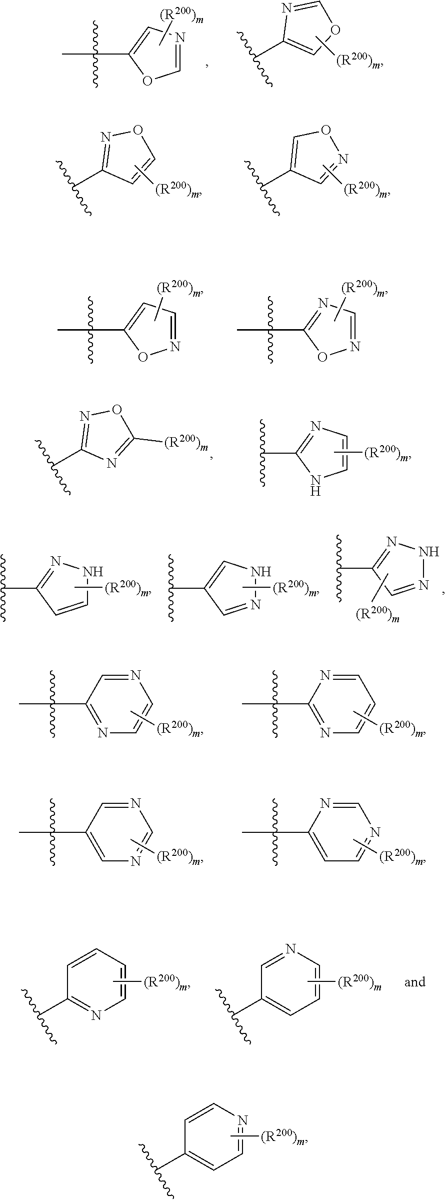
wherein m is 0, 1 or 2 as valence permits.

29. The compound of claim 25, or a pharmaceutically acceptable salt thereof, wherein R²⁰ is independently selected from: 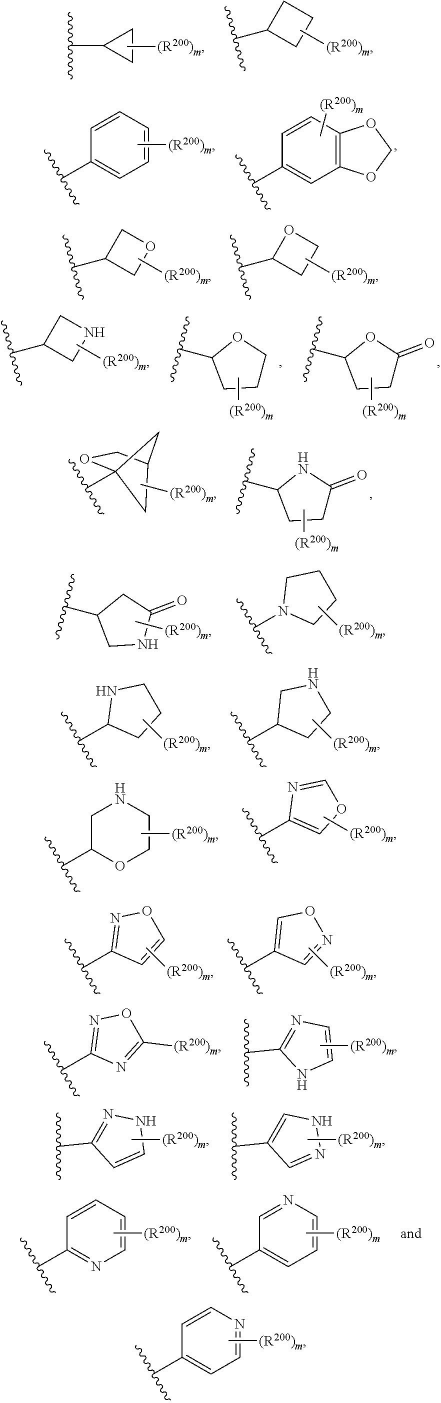
wherein m is 0, 1 or 2 as valence permits.

30. The compound of any one of claims 1-12, or a pharmaceutically acceptable salt thereof, wherein R² is selected from H, —F, —CH₃, —CH₂CH₃, —CH₂CH₂CH₃, —CF₂CH₂CH₃, —CH₂CH₂CH₂OCH₃, —CH(CH₃)₂, —CH₂CH₂CH₂N(CH₃)₂, —CF₂-cyclopropyl, cyclopropyl, —CH₂OCH₃, —OH, —OCH₃, —OCD₃, —OCHF₂, —OCH₂CH₃, —OCD₂CH₃, —OCD₂CD₃, —OCH₂CH₂F, —OCH₂CHF₂, —OCH₂CF₃, —OCH₂CH₂CH₂F, —OCH₂CH₂CH(CH₃)F, —OCH(CH₃)₂, —OCH₂CF(CH₃)₂, —OCH₂CH₂CH₂CH₃, —OCH₂CHFCH(CH₃)₂, —OCH₂CHF-cyclobutyl, —OCH₂CH₂OH, —OCH₂CH(OCH₃)CH₂CH₃, —OCH₂CH₂OCH₃, —OCH₂CH₂OCF₂H, —OCH₂CH₂OCH₂CH₃, —OCH₂CH₂CH₂OCH₃, —OCH₂CH(CH₃)OCH₃, —OCH₂CH(CH₃)CH₂OCH₃, —OCH(CH₃)CH₂OCH₃, —OCH₂CH(CH₃)OC(CH₃)₃, —OCH₂CH₂CH(CH₃)OCH₃, —OCH₂C(CH₃)₂OCH₃, —OCH₂CH₂OCH₂CH₃, —OCH₂CH₂OCH(CH₃)₂, —OCH₂CH₂OC(CH₃)₃, —OCH₂CH₂O-cyclopropyl, —OCH₂CH₂N(CH₃)₂, —OCH₂C(O)NHCH₃, —OCH₂C(O)N(CH₃)₂, —NH₂,

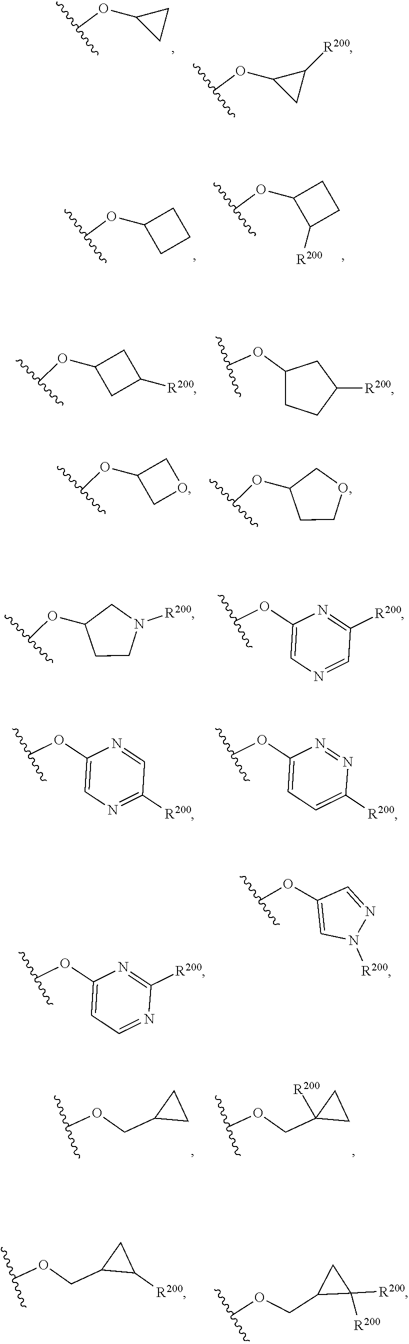

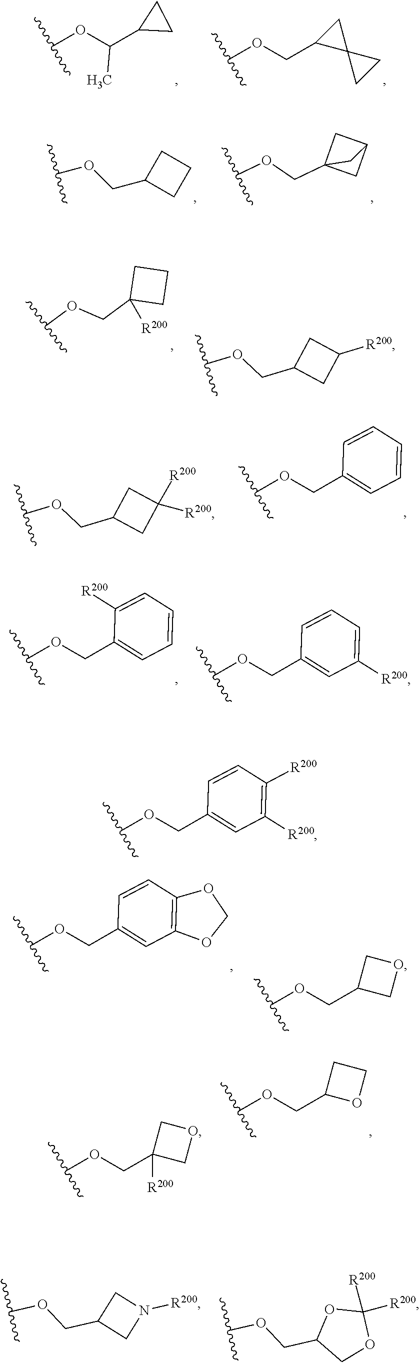

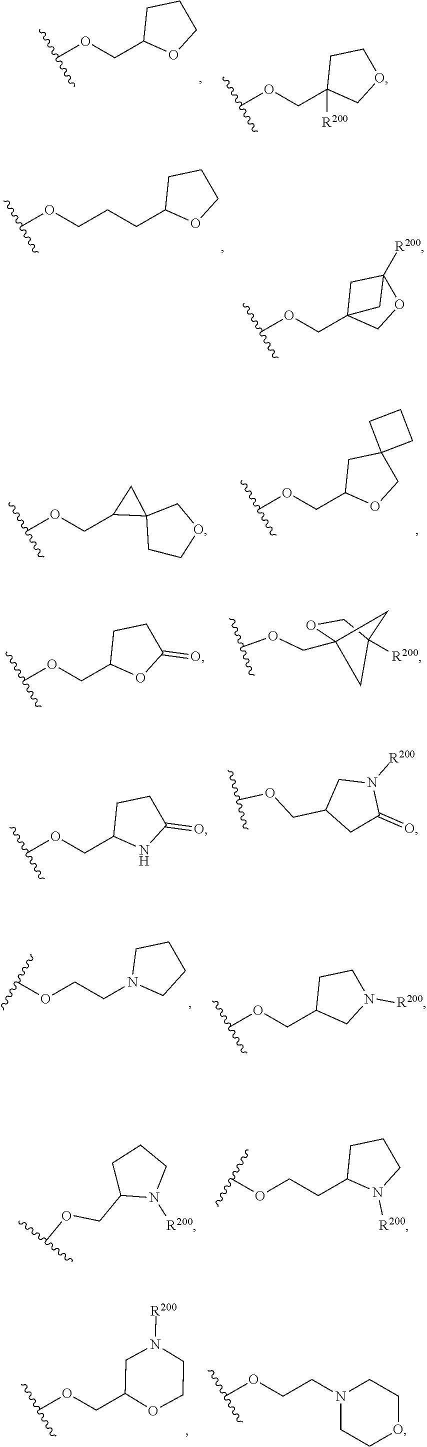

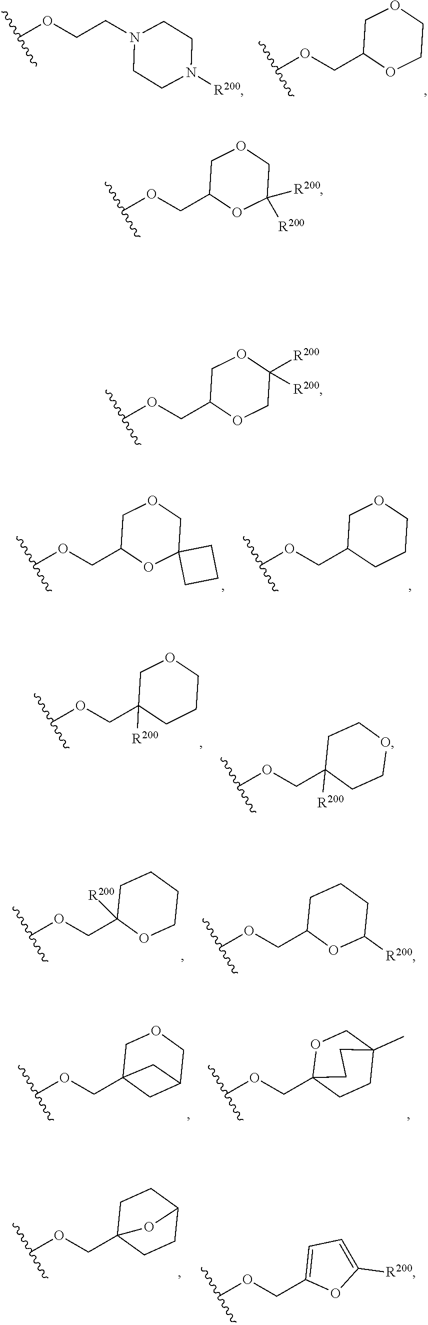

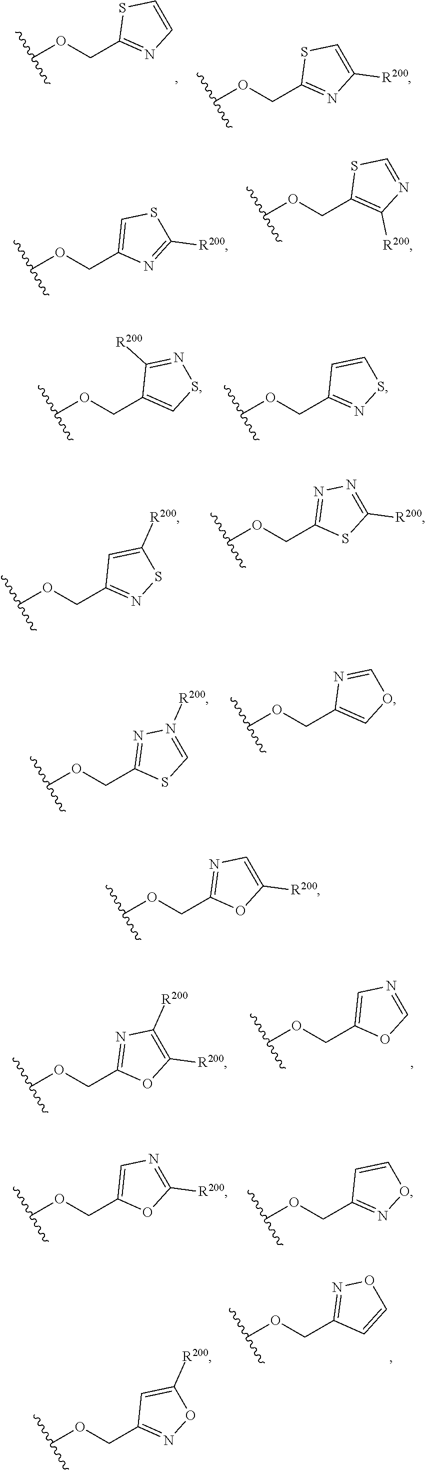

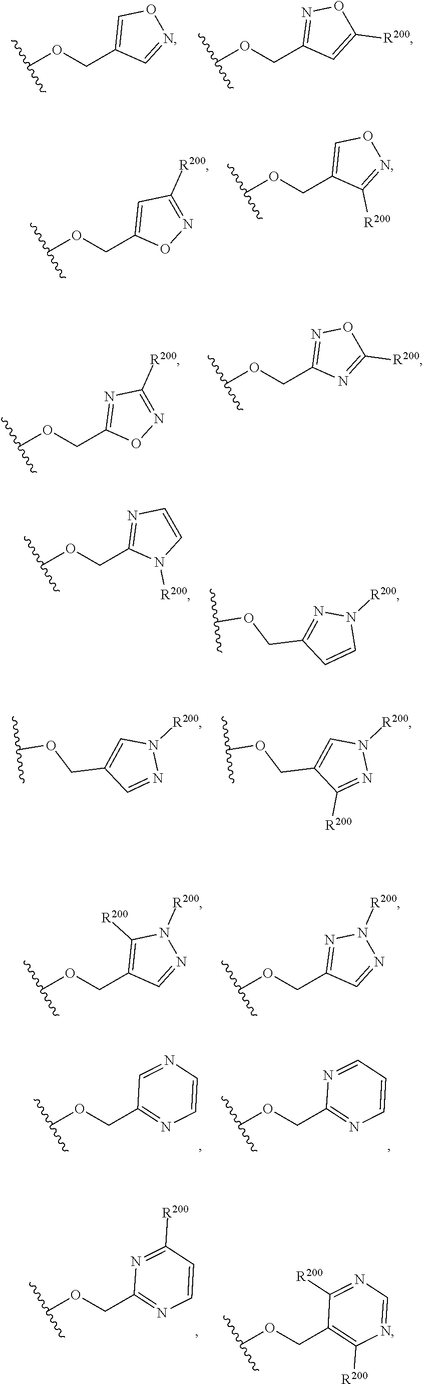

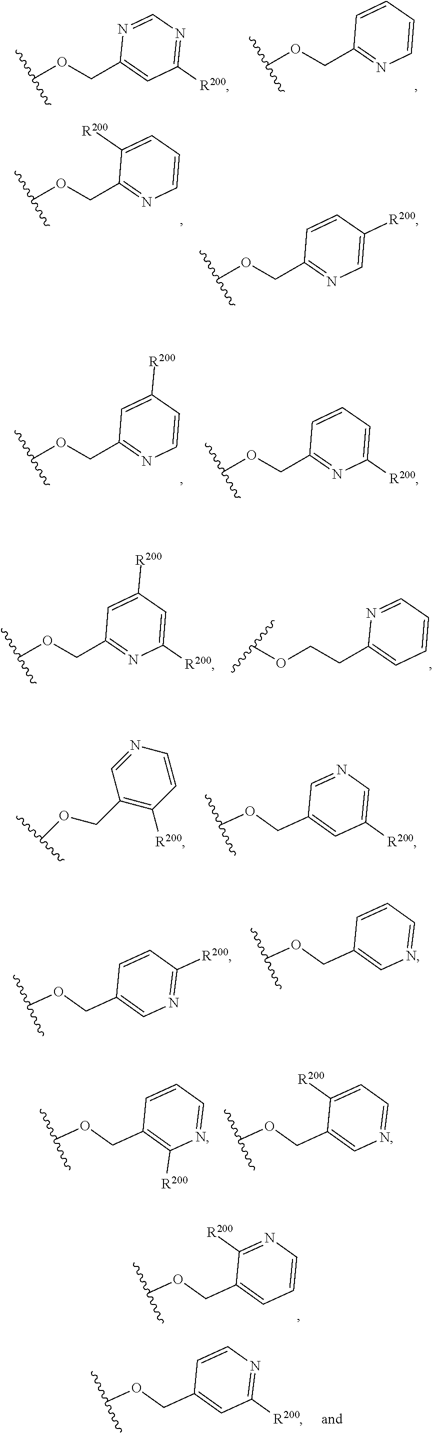

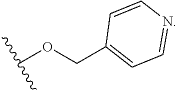 [SMILES: *OCc1ccncc1]

31. The compound of any one of claims 1-12, or a pharmaceutically acceptable salt thereof, wherein R² is selected from H, —CH₃, —CH₂CH₃, —CH₂CH₂CH₃, —CH₂CH₂CH₂OCH₃, —CH(CH₃)₂, —CH₂CH₂CH₂N(CH₃)₂, cyclopropyl, —CH₂OCH₃, OH, —OCH₃, —OCD₃, —OCHF₂, —OCH₂CH₃, —OCD₂CH₃, —OCD₂CD₃, —OCH₂CHF₂, —OCH₂CF₃, —OCH(CH₃)₂, —OCH₂CH₂OCH₃, —OCH₂CH₂OCF₂H, —OCH₂CH₂CH₂OCH₃, —OCH₂CH(CH₃)OCH₃, —OCH(CH₃)CH₂OCH₃, —OCH₂C(CH₃)₂OCH₃, —OCH₂CH₂OCH₂CH₃, —OCH₂CH₂OCH(CH₃)₂,

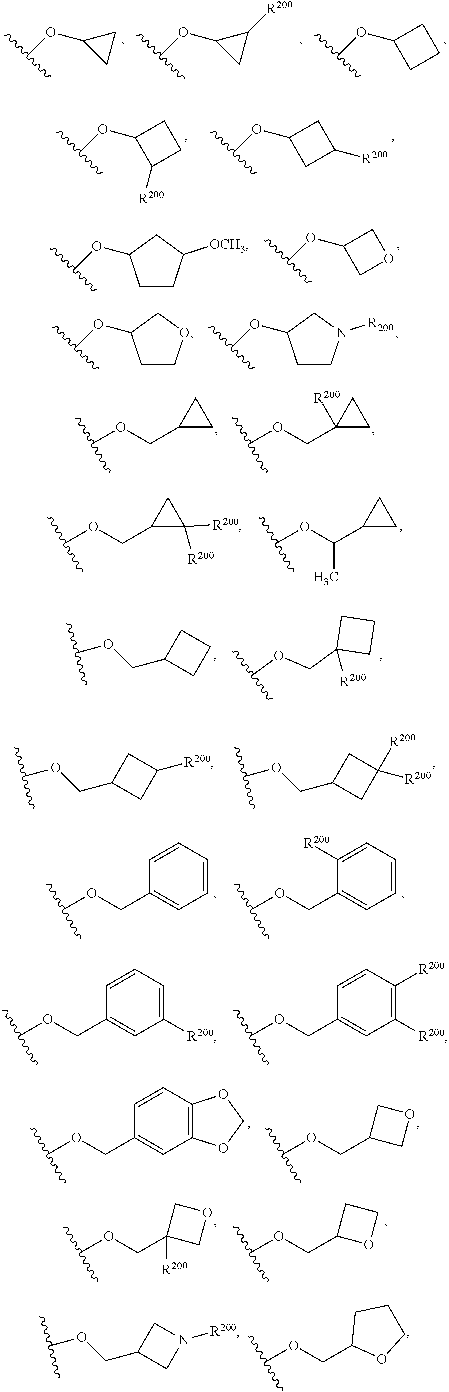

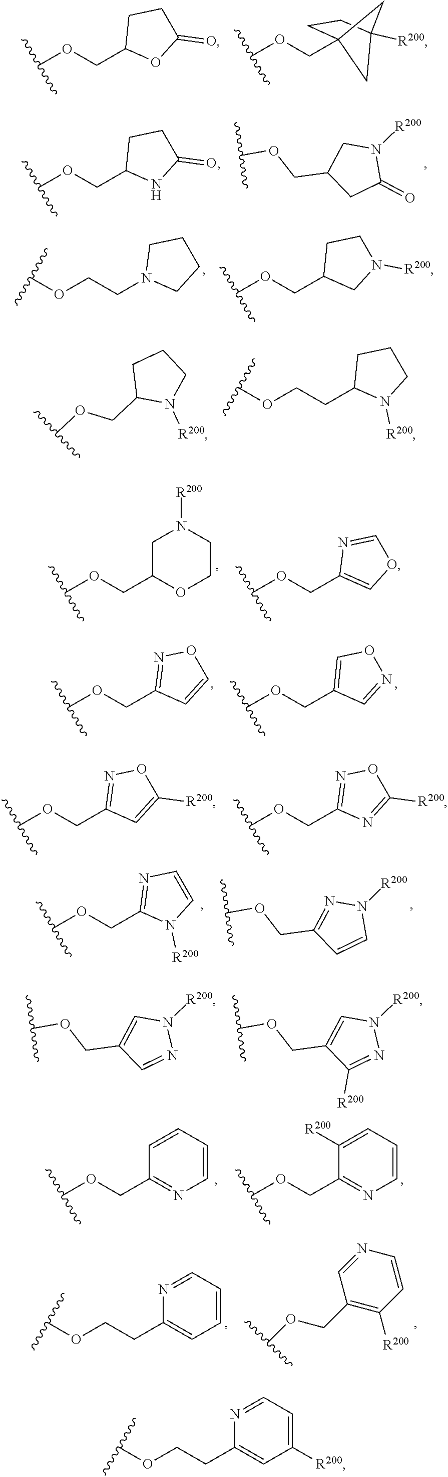

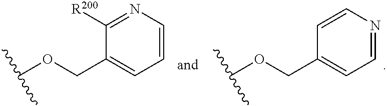

32. The compound of any one of claims 1-31, or a pharmaceutically acceptable salt thereof, wherein R²⁰⁰, for each occurrence, is independently selected from F, —CN, —Ch₃, —CH₂F, —CH₂CH₃, CH(CH₃)₂, —CH₂OCH₃, —OCH₃, cyclobutyl, and cyclopropyl.

33. The compound of any one of claims 1-31, or a pharmaceutically acceptable salt thereof, wherein R²⁰⁰, for each occurrence, is independently selected from F, —CN, —CH₃, —CF₃, —CH₂CH₃, —CH(CH₃)₂, —OCH₃ and cyclopropyl.

34. The compound of any one of claims 1-12, or a pharmaceutically acceptable salt thereof, wherein R² is C₃₋₆cycloalkyl, 4- to 6-membered monocyclic heterocyclyl, 7- to 10-membered bicyclic heterocyclyl or 5- to 6-membered monocyclic heteroaryl, each of which is optionally substituted with 1 to 2 R²⁰.

35. The compound of any one of claims 1-12, or a pharmaceutically acceptable salt thereof, wherein R² is selected from azetidinyl, cyclopropyl, tetrahydropyranyl, dioxino[2,3-d]pyridinyl, pyridazinonyl, pyrimidinonyl, pyrazinonyl, isoxazolyl, isothiazolyl, morpholinyl, oxaazabicyclo[3.1.1]heptanyl, oxazolyl, pyradazinyl, pyrazolyl, pyridinyl, pyrazinyl, pyridazinyl, triazinyl, pyrimidinyl, triazoyl, imidazolyl, oxazoyl, isoxazoyl, oxadiazoyl, pyrrolidinyl, thiadiazolyl, thiazolyl, 6,7-dihydro-[1,2,4]triazolo[1,5-a]pyrazin-8(5H)-onyl, 6,7-dihydro-4H-pyrazolo[5,1-c][1,4]oxazinyl, 5,6-dihydro-8H-imidazo[2,1-c][1,4]oxazinyl, 6,7-dihydro-5H-pyrazolo[5,1-b][1,3]oxazinyl, 4,5,6,7-tetrahydro-3H-imidazo[4,5-c]pyridinyl, 3,4,6,7-tetrahydropyrano[3,4-d]imidazolyl, 5,6,7,8-tetrahydroimidazo[1,2-a]pyrazinyl, 4,5,6,7-tetrahydropyrazolo[1,5-a]pyrimidinyl, 4,5,6,7-tetrahydropyrazolo[1,5-a]pyrazinyl, 5,6,7,8-tetrahydro-[1,2,4]triazolo[1,5-a]pyrazine, 5,6-dihydro-8H-[1,2,4]triazolo[5,1-c][1,4]oxazinyl, 5,6-dihydro-8H-imidazo[2,1-c][1,4]oxazinyl, imidazo[1,2-a]pyrimidinyl, [1,2,4]triazolo[4,3-a]pyrazinyl, pyrazolo[1,5-a]pyrimidinyl, [1,2,4]triazolo[1,5-a]pyrimidinyl, imidazo[1,2-b]pyridazinyl, 6,7-dihydro-5H-cyclopenta[b]pyridin-5-onyl, furo[3,4-d]pyrimidin-5(7H)-onyl, 5,7-dihydrofuro[3,4-d]pyrimidinyl, 7,8-dihydro-5H-pyrano[4,3-b]pyridinyl, 2,3-dihydro-[1,4]dioxino[2,3-b]pyridinyl, 5,6-dihydro-4H-pyrrolo[3,4-d]thiazolyl, 5,6-dihydro-4H-pyrrolo[1,2-b]pyrazolyl, and 2,3-dihydroimidazo[2,1-b]oxazolyl, each of which is optionally substituted with 1 to 2 R²⁰.

36. The compound of any one of claims 1-12, or a pharmaceutically acceptable salt thereof, wherein R² is selected from azetidinyl, cyclopropyl, dioxino[2,3-d]pyridinyl, isoxazolyl, isothiazolyl, morpholinyl, oxaazabicyclo[3.1.1]heptanyl, oxazolyl, pyradazinyl, pyrazolyl, pyridinyl, pyrazinyl, pyrimidinyl, triazoyl, oxazoyl, isoxazoyl, oxadiazoyl, pyrrolidinyl, thiadiazolyl and thiazolyl, each of which is optionally substituted with 1 to 2 R²⁰.

37. The compound of any one of claims 1-12, or a pharmaceutically acceptable salt thereof, wherein R² is selected from 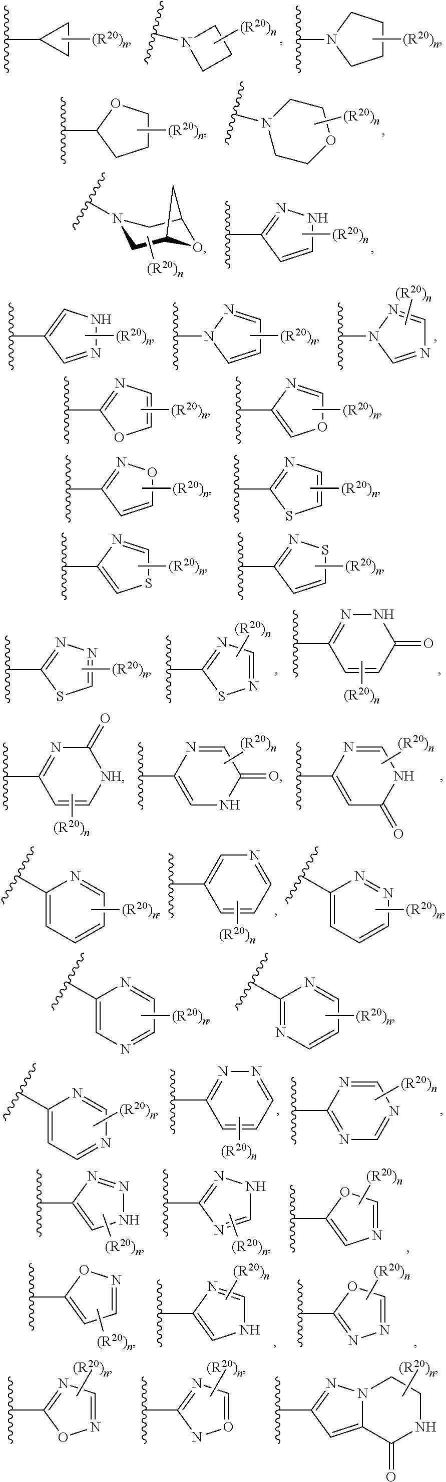 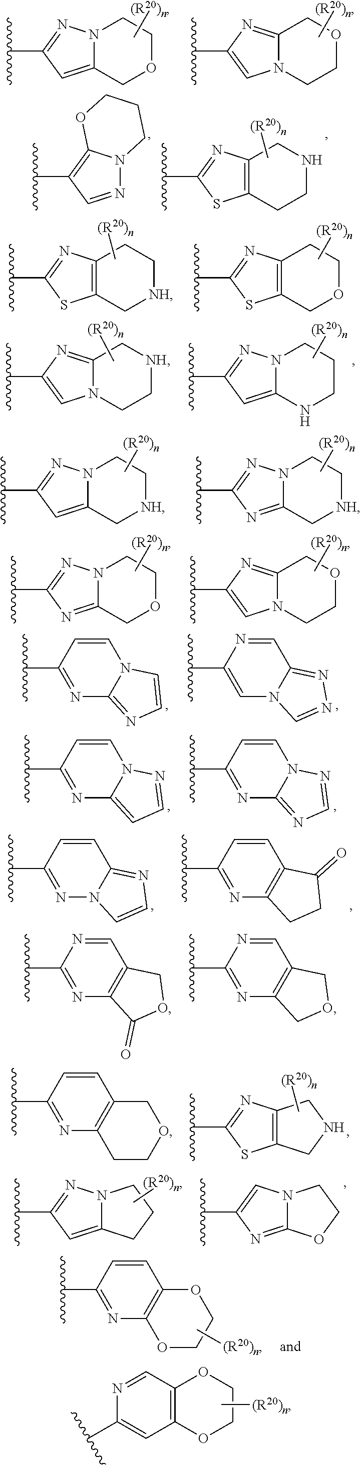
wherein n is 0, 1 or 2.

38. The compound of any one of claims 1-12 and 34-37, or a pharmaceutically acceptable salt thereof, wherein:
R²⁰, for each occurrence, is independently halo, —CN, C₁₋₄alkyl, C₁₋₄haloalkyl, OR^{20c}, —N(R^{20b})₂, —C(O)C₁₋₃alkyl, —SO₂C₁₋₃alkyl, P(O)(C₁₋₃alkyl)₂, C₃₋₆cycloalkyl, or 5- to 10-membered monocyclic or bicyclic heterocyclyl,
wherein the C₁₋₄alkyl represented by R²⁰ is optionally substituted by —CN, OH, —N(R^{20b})₂, C₁₋₃alkoxy, C₁₋₃haloalkoxy, C₃₋₆cycloalkyl, and 5- to 10-membered monocyclic or bicyclic heterocyclyl optionally substituted by C₁₋₄alkyl,
R^{20c} is H, C₁₋₄alkyl, C₁₋₄haloalkyl, or 4-membered monocyclic heterocyclyl, wherein the C₁₋₄alkyl is optionally substituted by C₁₋₃alkoxy;
wherein the 5- to 10-membered monocyclic or bicyclic heterocyclyl represented by R²⁰ is optionally substituted with C₁₋₄alkyl or C₁₋₃alkoxy;
each R^{20b} is, independently, H or C₁₋₄alkyl optionally substituted by C₁₋₃alkoxy.

39. The compound of any one of claims 1-12 and 34-37, or a pharmaceutically acceptable salt thereof, wherein R²⁰, for each occurrence, is independently halo, —CN, C₁₋₃alkyl, C₁₋₄ haloalkyl, C₁₋₃ alkoxy or C₃₋₆cycloalkyl.

40. The compound of claim 38, or a pharmaceutically acceptable salt thereof, wherein R²⁰, for each occurrence, is independently selected from —F, —Cl, —Br, —CN, —OH, —OCH₃, —OCHF₂, —OCH₂CH₃, —OCH(CH₃)₂, —OCH₂CH₂OCH₃, —CH₃, —CD₃, —CHF₂, —CH₂CH₃, —CH(CH₃)₂, —CF(CH₃)₂, —C(CH₃)₃, —CF₂CH₃, —CHFCH₃, —CH₂CH₂CH₃, —CH(CH₃)OH, —CH(CH₃)OCH₃, —CH₂CN, —CH₂N(CH₃)₂, —CH(CH₃)N(CH₃)₂, —CH₂CH₂OCH₃, —CH₂OCH₃, —CH₂OCHF₂, —CH₂N(CH₃)₂, —CH₂C(OH)(CH₃)₂, —CH₂C(OCH₃)(CH₃)₂, —CH₂CH₂OCH₂CH₃, —C(CH₃)₂OH, —C(CH₃)₂OCH₃, —C(CH₃)₂CN, —C(CH₃)₂N(CH₃)₂, —NHCH₃, —N(CH₃)₂, —NHCH(CH₃)₂, —NHCH₂CH₂OCH₃, —CH₂N(CH₃)CH₂CH₂OCH₃, —N(CH₃)CH₂CH₂OCH₃, —C(O)CH₃, SO₂CH₃, —SO₂CH₂CH₃, P(O)(CH₃)₂, 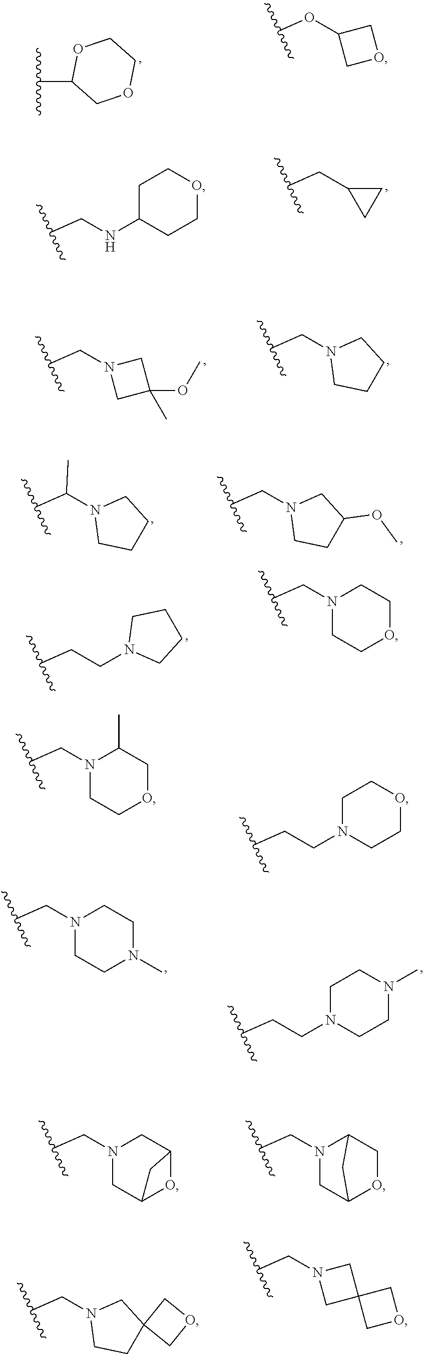 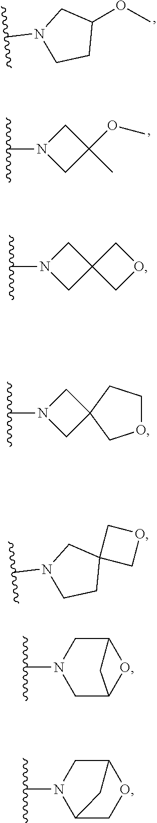
cyclopropyl, cyclobutyl, tetrahydrofuranyl, tetrahydropyranyl, azetidinyl, pyrrolidinyl, N-methylpiperazinyl, N-methylmorpholinyl, and morpholinyl.

41. The compound of claim 39, or a pharmaceutically acceptable salt thereof, wherein R²⁰, for each occurrence, is independently selected from F, —CN, —OCH₃, —CH₃, —CHF₂, cyclopropyl and cyclobutyl.

42. The compound of any one of claims 6-41, or a pharmaceutically acceptable salt thereof, wherein:
R³ is selected from H, halo, C₁₋₄alkyl, C₁₋₃haloalkyl, C₃₋₆cycloalkyl, C₂₋₄alkenyl, C₁₋₃ alkyl-C₁₋₃alkoxy, —OR^{3a}, —N(R^{3b})₂, phenyl, pyridinyl, pyrimidinyl, pyrazoyl, thiazoyl, indazoyl, [1,2,4]triazolo[1,5-a]pyridinyl, imidazo[1,2-a]pyridinyl, or imidazo[1,2-b]pyridazinyl, wherein the phenyl, pyridinyl, pyrimidinyl, pyrazoyl, thiazoyl, indazoyl, [1,2,4]triazolo[1,5-a]pyridinyl, imidazo[1,2-a]pyridinyl, or imidazo[1,2-b]pyridazinyl are each optionally substituted by 1 or 2 R^{3c};
R^{3a} is H, C₁₋₃alkyl, 4- to 8-membered monocyclic or bicycle heterocyclyl, or C₃₋₆ cycloalkyl, wherein the C₁₋₃alkyl and C₃₋₆cycloalkyl represented by R^{3a} are each optionally substituted with one or two substituents independently selected from halo, —CN, C₁₋₂alkyl, —OH and C₁₋₂alkoxy;
each R^{3b}, for each occurrence, is independently H, C₁₋₃alkyl, or C₃₋₅cycloalkyl;
each R^{3c} is independently selected from halo, oxo, —CN, —OR^{3a}, —N(R^{3b})₂, C₁₋₄alkyl, C₁₋₄alkyl-R^{3d}, C₁₋₄haloalkyl, —C(O)OR^{3a}, phenyl, cyclopropyl, cyclobutyl, oxetanyl, or morpholinyl;
R^{3d} is —C(O)OR^{3a}, —N(R^{3b})₂, —OR^{3a}, cyclopropyl, or morpholinyl.

43. The compound of any one of claims 6-41, or a pharmaceutically acceptable salt thereof, wherein:
R³ is selected from H, C₁₋₄alkyl, C₁₋₃haloalkyl, C₃₋₆cycloalkyl, —OR^{3a}, —N(R^{3b})₂, phenyl, pyridinyl, pyrimidinyl, pyrazoyl, thiazoyl, indazoyl, [1,2,4]triazolo[1,5-a]pyridinyl, imidazo[1,2-a]pyridinyl, or imidazo[1,2-b]pyridazinyl, wherein the phenyl, pyridinyl, pyrimidinyl, pyrazoyl, thiazoyl, indazoyl, [1,2,4]triazolo[1,5-a]pyridinyl, imidazo[1,2-a]pyridinyl, or imidazo[1,2-b]pyridazinyl are each optionally substituted by 1 or 2 R^{3c};
R^{3a} is H, C₁₋₃alkyl, or C₃₋₆cycloalkyl, wherein the C₁₋₃alkyl and C₃₋₆cycloalkyl represented by R^{3a} are each optionally substituted with one or two substituents independently selected from halo, —CN, C₁₋₂alkyl, —OH and C₁₋₂alkoxy;
each R^{3b}, for each occurrence, is independently H, C₁₋₃alkyl, or C₃₋₅cycloalkyl;
each R^{3c} is independently selected from halo, oxo, —CN, —OR^{3a}, —N(R^{3b})₂, C₁₋₄alkyl, C₁₋₄alkyl-R^{3d}, C₁₋₄haloalkyl, —C(O)OR^{3a}, phenyl, cyclopropyl, cyclobutyl, oxetanyl, or morpholinyl;
R^{3d} is —C(O)OR^{3a}, —N(R^{3b})₂, —OR^{3a}, cyclopropyl, or morpholinyl.

44. The compound of claim 42, or a pharmaceutically acceptable salt thereof, wherein R³ is selected from H, —F, —Cl, —CH₃, —CH₂CH₃, —CF₂CH₃, —CF₃, —CH(CH₃)₂, cyclopropyl, —CH═CH₂, —CH₂OCH₃, —OCH₃, —OCH₂CH₃, —OCH(CH₃)₂, —OCH₂CH₂OCH₃, —OCH₂CH₂CH₂OCH₃, —NHCH₃, 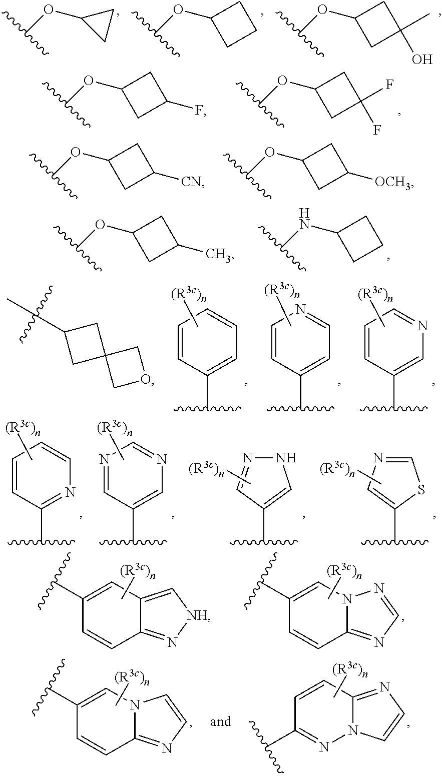
wherein n is 0, 1, or 2.

45. The compound of claim 43, or a pharmaceutically acceptable salt thereof, wherein R³ is selected from H, —CH₃, —CH₂CH₃, —CF₂CH₃, —CH(CH₃)₂, cyclopropyl, —OCH₃, —OCH₂CH₂—OCH₃, —NHCH₃, 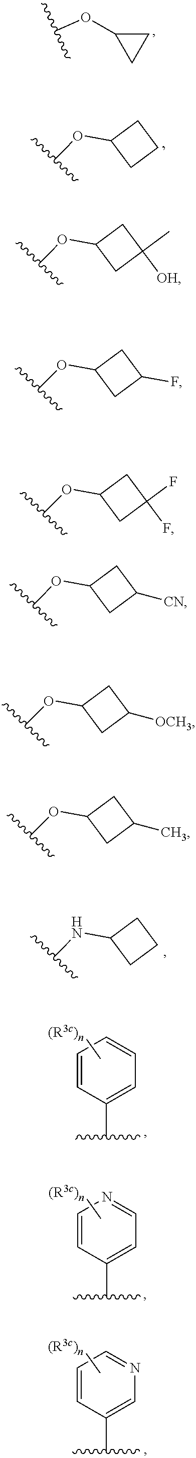 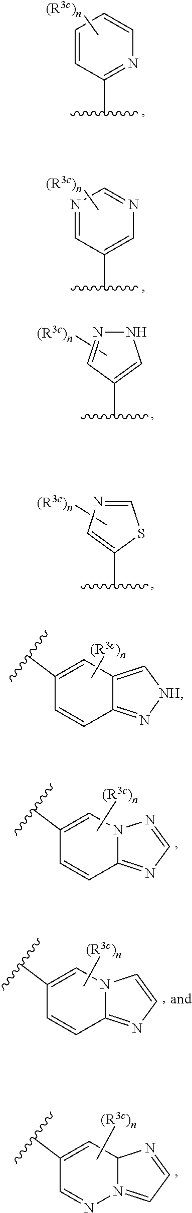
wherein n is 0, 1, or 2.

46. The compound of any one of claims 6-45, or a pharmaceutically acceptable salt thereof, wherein each R^{3c} is individually selected from —CN, —F, —Cl, —OCH₃, —CH₃, —CH₂CH₃, —CH(CH₃)₂, —CH₂CH(CH₃)₂, —CHF₂, —CH₂CF₃, —CF₃, —CD₃, —CH₂CH₂OCH₃, —CH₂— cyclopropyl, —CH₂CH₂-morpholinyl, cyclopropyl, cyclobutyl, —CH₂C(O)OH, —C(O)OC(CH₃)₃, —CH₂CH₂N(CH₃)₂, oxetanyl, and morpholinyl.

47. The compound of any one of claims 6-45, or a pharmaceutically acceptable salt thereof, wherein each R^{3c} is individually selected from —CN, F, —OCH₃, —CH₃, —CH₂CH₃, —CH(CH₃)₂, —CH₂CH(CH₃)₂, —CHF₂, —CH₂CF₃, —CF₃, —CD₃, —CH₂CH₂OCH₃, —CH₂-cyclopropyl, —CH₂CH₂-morpholinyl, cyclopropyl, cyclobutyl, —CH₂C(O)OH, —C(O)OC(CH₃)₃, —CH₂CH₂N(CH₃)₂, and morpholinyl.

48. The compound of any one of claims 6-47, or a pharmaceutically acceptable salt thereof, wherein R⁴ is selected from C₁₋₄haloalkyl, C₁₋₃alkoxy, C₁₋₃alkoxy-C₁₋₃alkoxy, C₁₋₃haloalkoxy, —C₂₋₄alkenyl, C₂₋₄haloalkenyl, 5- to 7 membered monocyclic or bicyclic heterocyclyl, and C₃₋₆cycloalkyl, wherein the 5- to 7 membered monocyclic or bicyclic heterocyclyl, and C₃₋₆cycloalkyl are each optionally substituted with 1 to 3 substituents independently selected from halo, C₁₋₃haloalkyl, C₁₋₃alkyl-C₁₋₃alkoxy, and C₁₋₃alkyl.

49. The compound of any one of claims 6-47, or a pharmaceutically acceptable salt thereof, wherein R⁴ is selected from C₁₋₄haloalkyl and C₃₋₆cycloalkyl optionally substituted with 1 to 3 substituents independently selected from halo and C₁₋₃alkyl.

50. The compound of claim 48, or a pharmaceutically acceptable salt thereof, wherein R⁴ is selected from —CF₂CH₃, —CF₂CFH₂, —CFHCFH₂, —CF(CH₃)₂, —CF(CH₃)CFH₂, —CH(CH₃)CFH₂, —CF₂CH₂CH₃, —CF(CH₃)₂, —OCH₃, —OCHF₂, —OCH₂CH₂OCH₃, —CF=CH₂,

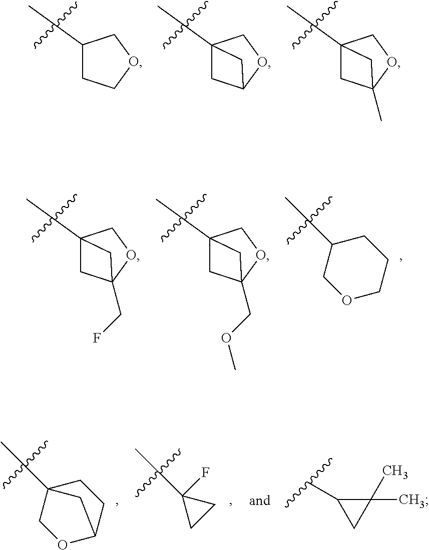

51. The compound of claim 49, or a pharmaceutically acceptable salt thereof, wherein R⁴ is selected from —CF₂CH₃, —CF₂CFH₂, —CFHCFH₂, —CF₂CH₂CH₃, —CF(CH₃)₂, and

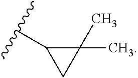 [SMILES: *C1CC1(C)C]

52. The compound of any one of claims 6-51, or a pharmaceutically acceptable salt thereof, wherein R⁵ is H or 5-membered heteroaryl having 1 to 3 heteroatoms independently selected from nitrogen, oxygen and sulfur, wherein the 5-membered heteroaryl represented by R⁵
is optionally substituted by 1 to 3 R⁵⁰.

53. The compound of any one of claims 6-51, or a pharmaceutically acceptable salt thereof, wherein R⁵ is H or pyrazolyl optional substituted by 1 to 3 R⁵⁰.

54. The compound of any one of claims 1-53 or a pharmaceutically acceptable salt thereof, wherein R⁶ is H, halo, C₁₋₃alkoxy.

55. The compound of claim 54, or a pharmaceutically acceptable salt thereof, wherein R⁶ is H, —F, or —OCH₃.

56. The compound of any one of claims 1 to 55, or a pharmaceutically acceptable salt thereof, wherein R⁷ is H.

57. The compound of claim 1, wherein the compound is represented by Formula (IV-1) or (V-1): 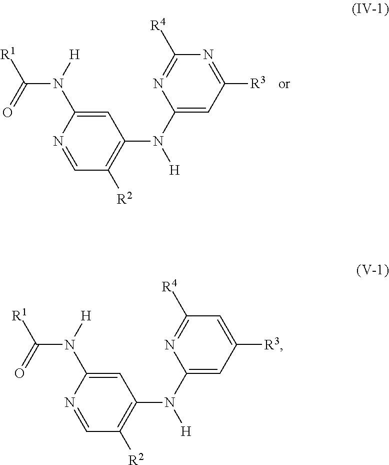
or a pharmaceutically acceptable salt thereof, wherein:
R¹ is C₁₋₃alkyl;
R² is —OR^{2a} or 5-membered monocyclic heteroaryl optionally substituted with C₁₋₃alkyl;
R^{2a} is C₁₋₄alkyl optionally substituted with R²⁰;
R²⁰ is C₁₋₃alkoxy or C₃₋₆cycloalkyl optionally substituted with C₁₋₂alkoxy;
R³ is selected from H, —OR^{3a}, C₁₋₃alkyl, C₃₋₆cycloalkyl, and pyrazoyl, wherein the pyrazoyl is optionally substituted by 1 or 2 R^{3c};
R^{3a} is C₁₋₃alkyl optionally substituted with C₁₋₃alkoxy, or C₃₋₆cycloalkyl optionally substituted with 1 or 2 substituents independently selected from C₁₋₃alkoxy, C₁₋₃alkyl and —OH;
R^{3c} is C₁₋₃alkyl; and
R⁴ is C₁₋₃haloalkyl.

58. The compound of claim 57, or a pharmaceutically acceptable salt thereof, wherein R¹ is —CH₃.

59. The compound of claim 57 or 58, or a pharmaceutically acceptable salt thereof, wherein R² is selected from —OCH₃, —OCD₃, —OCH₂CH₃, —OCD₂CH₃, —OCD₂CD₃, —OCH₂CH₂OCH₃,

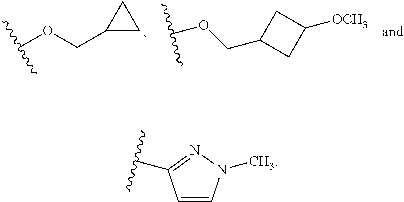

60. The compound of any one of claims 57-59, or a pharmaceutically acceptable salt thereof, wherein R³ is selected from H, —CH₃, —CH₂CH₃, cyclopropyl, —OCH₃, —OCH₂CH₂OCH₃,

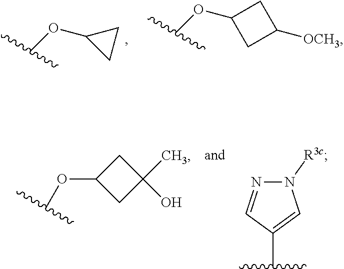

61. The compound of any one of claims 57-60, or a pharmaceutically acceptable salt thereof, wherein R^{3c} is —CH₃; and R⁴ is —CF₂CH₃, —CF₂CFH₂, —CFHCFH₂, —CF₂CH₂CH₃, —CF(CH₃)₂.

62. The compound of any one of claims 1-4, wherein the compound is represented by Formula (III): 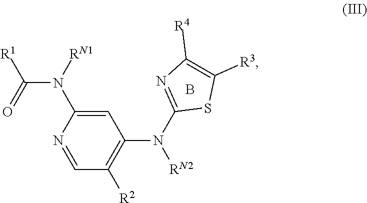
or a pharmaceutically acceptable salt thereof, wherein:
R¹ is C₁₋₆alkyl;
R² is C₁₋₄alkoxy;
R³ is H or C₁₋₆alkyl;
R⁴ is C₁₋₄haloalkyl or 4- to 6-membered monocyclic heterocyclyl having 1 to 2 heteroatoms independently selected from nitrogen and oxygen; and
R^{N1} and R^{N2} are each independently H or C₁₋₃alkyl.

63. The compound of claim 62, or a pharmaceutically acceptable salt thereof, wherein R¹ is —CH₃.

64. The compound of claim 62 or 63, or a pharmaceutically acceptable salt thereof, wherein R² is —OCH₂CH₃ or —OCH₂CH₂OCH₃.

65. The compound of any one of claims 62-64, or a pharmaceutically acceptable salt thereof, wherein R³ is H or —CH₃.

66. The compound of any one of claims 62-65, or a pharmaceutically acceptable salt thereof, wherein R⁴ is C₁₋₃haloalkyl or tetrahydrofuranyl.

67. The compound of claim 66, or a pharmaceutically acceptable salt thereof, wherein R⁴ is —CF₂CH₃ or

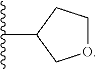 [SMILES: *C1CCOC1]

68. The compound of claim 1, wherein the compound is represented by Formula (X): 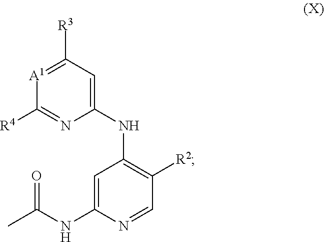
or a pharmaceutically acceptable salt thereof, wherein:
A¹ is N or CH;
R² is —OR^{2a}, 5- or 6-membered monocyclic heteroaryl, or 7- to 10-membered bicyclic heterocyclyl, wherein the 5- or 6-membered monocyclic heteroaryl or 7- to 10-membered bicyclic heterocyclyl are each optionally substituted with one or two R²⁰;
R^{2a} is C₁₋₃alkyl optionally substituted with C₁₋₃alkoxy;
R²⁰ is C₁₋₃alkyl optionally substituted by —N(C₁₋₃alkyl)₂;
R³ is H, C₁₋₃alkyl, or —OR³a;
R^{3a} is C₃₋cycloalkyl;
R⁴ is C₁₋₄haloalkyl or 5- to 7-membered bicyclic heterocyclyl.

69. The compound of claim 68, or a pharmaceutically acceptable salt thereof, wherein A¹ is N.

70. The compound of claim 68 or 69, or a pharmaceutically acceptable salt thereof, wherein R² is —OCH₃, —OCH₂CH₃, or —OCH₂CH₂OCH₃.

71. The compound of claim 68 or 69, or a pharmaceutically acceptable salt thereof, wherein R² is pyrazoyl pyridinyl, pyrimidinyl, or 4,5,6,7-tetrahydropyrazolo[1,5-a]pyrazinyl, each of which is optionally substituted by one or two R²⁰.

72. The compound of claim 71, or a pharmaceutically acceptable salt thereof, wherein R² is 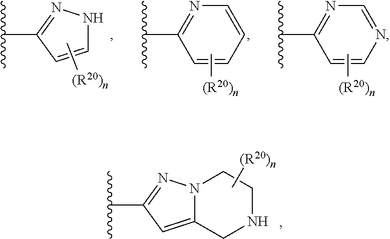
wherein n is 0, 1 or 2.

73. The compound of claim 72, or a pharmaceutically acceptable salt thereof, wherein R² is

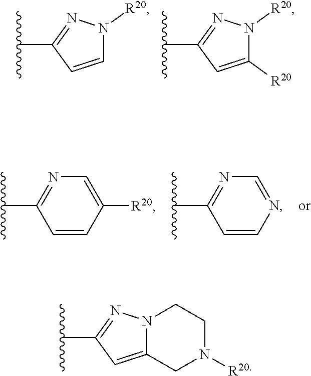

74. The compound of any one of claims 68, 69, or 71-73, or a pharmaceutically acceptable salt thereof, wherein each R²⁰ is, independently, —CH₃, —CH₂CH₃, or —CH₂N(CH₃)₂.

75. The compound of any one of claims 68-74, or a pharmaceutically acceptable salt thereof, wherein R³ is H, —CH₃, —CH₂CH₃, or —O-cyclopropyl.

76. The compound of any one of claims 68-75, or a pharmaceutically acceptable salt thereof, wherein R⁴ is —CF₂CH₃ or

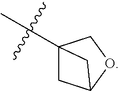 [SMILES: *C12COC(C1)C2]

77. The compound of claim 1, selected from a compound of any one of Examples 1-923 or a pharmaceutically acceptable salt thereof.

78. A pharmaceutical composition comprising a compound or a pharmaceutically acceptable salt thereof of any one of claims 1-77 and a pharmaceutically acceptable carrier.

79. A method of inhibiting tyrosine kinase 2 (TYK2) activity in a subject in need thereof comprising administering to the subject an effective amount of a compound according to any one of claims 1-77 or a pharmaceutically acceptable salt thereof or a pharmaceutical composition according to claim 78.

80. A method of treating a disease or disorder responsive to inhibition of tyrosine kinase 2 (TYK2) in a subject comprising administering to the subject an effective amount of a compound according to any one of claims 1-77 or a pharmaceutically acceptable salt thereof or a pharmaceutical composition according to claim 78.

81. The method of claim 80, wherein the disease or disorder is inflammation, autoimmune disease, neuroinflammation, arthritis, rheumatoid arthritis, spondyloarthropathies, systemic lupus erythematous, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn.
